SEQUENCE LISTING (186 sequences)

SEQ ID NO: 1 - DNA, 1943 bp, Homo sapiens
agtgcgctgtgctcgaggggtgccggccaggcctgagcgagcgagctagccagcaggcatcgagggggcgcggctgccgtccggacgagacaggcgaacccgacgcagaagagtccaccaccggacagccaggtagccgccgcgtccctcgcacacgcagagtcgggcggcgcggggtctcccttgcgcccggcctccgccctctcctcctctcctttccccttcttctcgctgtcctctcctctctcgctgcccgcgtttgcgcagccccgggccatgtccgacgcgtccctccgcagcacatccacgatggagcgtcttgtcgcccgtgggaccttcccagtactagtgcgcaccagcgcctgccgcagcctcttcgggccggtggaccacgaggagctgagccgcgagctgcaggcccgcctggccgagctgaacgccgaggaccagaaccgctgggattacgacttccagcaggacatgccgctgcggggccctggacgcctgcagtggaccgaagtggacagcgactcggtgcccgcgttctaccgcgagacggtgcaggtggggcgctgccgcctgctgctggcgccgcggcccgtcgcggtcgcggtggctgtcagcccgcccctcgagccggccgctgagtccctcgacggcctcgaggaggcgccggagcagctgcctagtgtcccggtcccggccccggcgtccaccccgcccccagtcccggtcctggctccagccccggccccggctccggctccggtcgcggctccggtcgcggctccggtcgcggtcgcggtcctggccccggccccggccccggctccggctccggctccggccccggctccagtcgcggccccggccccagccccggccccggccccggccccggcccccgccccggccccggccccggacgcggcgcctcaagagagcgccgagcagggcgcgaaccaggggcagcgcggccaggagcctctcgctgaccagctgcactcggggatttcgggacgtcccgcggccggcaccgcggccgccagcgccaacggcgcggcgatcaagaagctgtccgggcctctgatctccgatttcttcgccaagcgcaagagatcagcgcctgagaagtcgtcgggcgatgtccccgcgccgtgtccctctccaagcgccgcccctggcgtgggctcggtggagcagaccccgcgcaagaggctgcggtgagccaatttagagcccaaagagccccgagggaacctgccggggcagcggacgttggaagggcgctgggcctcggctgggaccgttcatgtagcagcaaccggcggcggctgccgcagagcagcgttcggttttgtttttaaattttgaaaactgtgcaatgtattaataacgtctttttatatctaaatgtattctgcacgagaaggtacactggtcccaaggtgtaaagctttaagagtcatttatataaaatgtttaatctctgctgaaactcagtgcaaaaaaaagaaaaaagaaaaaaaaaaggaaaaaataaaaaaaccatgtatatttgtacaaaaagtttttaaagttatactaacttatattttctatttatgtccaggcgtggaccgctctgccacgcactagctcggttattggttatgccaaaggcactctccatctcccacatctggttattgacaagtgtaactttattttcatcgcggactctggggaagggggtcactcacaagctgtagctgccatacatgcccatctagcttgcagtctcttcgcgctttcgctgtctctcttattatgactgtgtttatctgaaacttgaagacaagtctgttaaaatggttcctgagccgtctgtaccactgccccggcccctcgtccgccgggttctaaataaagaggccgaaaaatgctgcaaaaaaaaaaaaaaa

SEQ ID NO: 2 - DNA, 3978 bp, Homo sapiens
acccactcccgctgccccgtccggcccgcgccgcttcctcgcagcagctgctcccggctccgcggccgcagcccgcgtggacgctccgagcgccccccgacggacgggaccggctccctggcggtcgggcgagcgggcggcaacgctgcccggccggcagcgctggggttaagtggcccaagtaaacctagctcggcgatcggcgccggagattcgcgagcccagcgccctgcacggccgccagccggcctcccgccagccagccccgacccgcggctccgccgcccagccgcgccccagccagccctgcggcaggaaagcatgaagggagacaccaggcatctcaatggagaggaggacgccggcgggagggaagactcgatcctcgtcaacggggcctgcagcgaccagtcctccgactcgcccccaatcctggaggctatccgcaccccggagatcagaggccgaagatcaagctcgcgactctccaagagggaggtgtccagtctgctaagctacacacaggacttgacaggcgatggcgacggggaagatggggatggctctgacaccccagtcatgccaaagctcttccgggaaaccaggactcgttcagaaagcccagcttccctgagacggcgggcaacagcatcggcaggaacgccatggccgtcccctcccagctcttaccttaccatcgacctcacagacgacacagaggacacacatgggacgccccagagcagcagtaccccctacgcccgcctagcccaggacagccagcaggggggcatggagtccccgcaggtggaggcagacagtggagatggagacagttcagagtatcaggatgggaaggagtttggaataggggacctcgtgtggggaaagatcaagggcttctcctggtggcccgccatggtggtgtcttggaaggccacctccaagcgacaggctatgtctggcatgcggtgggtccagtggtttggcgatggcaagttctccgaggtctctgcagacaaactggtggcactggggctgttcagccagcactttaatttggccaccttcaataagctcgtctcctatcgaaaagccatgtaccatgctctggagaaagctagggtgcgagctggcaagaccttccccagcagccctggagactcattggaggaccagctgaagcccatgttggagtgggcccacgggggcttcaagcccactgggatcgagggcctcaaacccaacaacacgcaaccagagaacaagactcgaagacgcacagctgacgactcagccacctctgactactgccccgcacccaagcgcctcaagacaaattgctataacaacggcaaagaccgaggggatgaagatcagagccgagaacaaatggcttcagatgttgccaacaacaagagcagcctggaagatggctgtttgtcttgtggcaggaaaaaccccgtgtccttccaccctctctttgagggggggctctgtcagacatgccgggatcgcttccttgagctgttttacatgtatgatgacgatggctatcagtcttactgcactgtgtgctgcgagggccgagagctgctgctttgcagcaacacgagctgctgccggtgtttctgtgtggagtgcctggaggtgctggtgggcacaggcacagcggccgaggccaagcttcaggagccctggagctgttacatgtgtctcccgcagcgctgtcatggcgtcctgcggcgccggaaggactggaacgtgcgcctgcaggccttcttcaccagtgacacggggcttgaatatgaagcccccaagctgtaccctgccattcccgcagcccgaaggcggcccattcgagtcctgtcattgtttgatggcatcgcgacaggctacctagtcctcaaagagttgggcataaaggtaggaaagtacgtcgcttctgaagtgtgtgaggagtccattgctgttggaaccgtgaagcacgaggggaatatcaaatacgtgaacgacgtgaggaacatcacaaagaaaaatattgaagaatggggcccatttgacttggtgattggcggaagcccatgcaacgatctctcaaatgtgaatccagccaggaaaggcctgtatgagggtacaggccggctcttcttcgaattttaccacctgctgaattactcacgccccaaggagggtgatgaccggccgttcttctggatgtttgagaatgttgtagccatgaaggttggcgacaagagggacatctcacggttcctggagtgtaatccagtgatgattgatgccatcaaagtttctgctgctcacagggcccgatacttctggggcaacctacccgggatgaacaggatctttggctttcctgtgcactacacagacgtgtccaacatgggccgtggtgcccgccagaagctgctgggaaggtcctggagcgtgcctgtcatccgacacctcttcgcccctctgaaggactactttgcatgtgaatagttccagccaggccccaagcccactggggtgtgtggcagagccaggacccaggaggtgtgattcctgaaggcatccccaggccctgctcttcctcagctgtgtgggtcataccgtgtacctcagttccctcttgctcagtgggggcagagccacctgactcttgcaggggtagcctgaggtgccgcctccttgtgcacaaatcagacctggctgcttggagcagcctaacacggtgctcattttttcttctcctaaaactttaaaacttgaagtaggtagcaacgtggctttttttttttcccttcctgggtctaccactcagagaaacaatggctaagataccaaaaccacagtgccgacagctctccaatactcaggttaatgctgaaaaatcatccaagacagttattgcaagagtttaatttttgaaaactggctactgctctgtgtttacagacgtgtgcagttgtaggcatgtagctacaggacatttttaagggcccaggatcgttttttcccagggcaagcagaagagaaaatgttgtatatgtcttttacccggcacattccccttgcctaaatacaagggctggagtctgcacgggacctattagagtattttccacaatgatgatgatttcagcagggatgacgtcatcatcacattcagggctattttttcccccacaaacccaagggcaggggccactcttagctaaatccctccccgtgactgcaatagaaccctctggggagctcaggaaggggtgtgctgagttctataatataagctgccatatattttgtagacaagtatggctcctccatatctccctcttccctaggagaggagtgtgaagcaaggagcttagataagacaccccctcaaacccattccctctccaggagacctaccctccacaggcacaggtccccagatgagaagtctgctaccctcatttctcatctttttactaaactcagaggcagtgacagcagtcagggacagacatacatttctcataccttccccacatctgagagatgacagggaaaactgcaaagctcggtgctccctttggagattttttaatccttttttattccataagaagtcgtttttagggagaacgggaattcagacaagctgcatttcagaaatgctgtcataatggtttttaacaccttttactcttcttactggtgctattttgtagaataaggaacaacgttgacaagttttgtggggctttttatacactttttaaaatctcaaacttctatttttatgtttaacgttttcattaaaattttttttgtaactggagccacgacgtaacaaatatggggaaaaaactgtgccttgtttcaacagtttttgctaatttttaggctgaaagatgacggatgcctagagtttaccttatgtttaattaaaatcagtatttgtctaaaaaaaaaaaaaaaaaa

SEQ ID NO: 3 - DNA, 3136 bp, Homo sapiens
gcgcagaacttggggagccgccgccgccatccgccgccgcagccagcttccgccgccgcaggaccggcccctgccccagcctccgcagccgcggcgcgtccacgcccgcccgcgcccagggcgagtcggggtcgccgcctgcacgcttctcagtgttccccgcgccccgcatgtaacccggccaggcccccgcaactgtgtcccctgcagctccagccccgggctgcacccccccgccccgacaccagctctccagcctgctcgtccaggatggccgcggccaaggccgagatgcagctgatgtccccgctgcagatctctgacccgttcggatcctttcctcactcgcccaccatggacaactaccctaagctggaggagatgatgctgctgagcaacggggctccccagttcctcggcgccgccggggccccagagggcagcggcagcaacagcagcagcagcagcagcgggggcggtggaggcggcgggggcggcagcaacagcagcagcagcagcagcaccttcaaccctcaggcggacacgggcgagcagccctacgagcacctgaccgcagagtcttttcctgacatctctctgaacaacgagaaggtgctggtggagaccagttaccccagccaaaccactcgactgccccccatcacctatactggccgcttttccctggagcctgcacccaacagtggcaacaccttgtggcccgagcccctcttcagcttggtcagtggcctagtgagcatgaccaacccaccggcctcctcgtcctcagcaccatctccagcggcctcctccgcctccgcctcccagagcccacccctgagctgcgcagtgccatccaacgacagcagtcccatttactcagcggcacccaccttccccacgccgaacactgacattttccctgagccacaaagccaggccttcccgggctcggcagggacagcgctccagtacccgcctcctgcctaccctgccgccaagggtggcttccaggttcccatgatccccgactacctgtttccacagcagcagggggatctgggcctgggcaccccagaccagaagcccttccagggcctggagagccgcacccagcagccttcgctaacccctctgtctactattaaggcctttgccactcagtcgggctcccaggacctgaaggccctcaataccagctaccagtcccagctcatcaaacccagccgcatgcgcaagtaccccaaccggcccagcaagacgcccccccacgaacgcccttacgcttgcccagtggagtcctgtgatcgccgcttctcccgctccgacgagctcacccgccacatccgcatccacacaggccagaagcccttccagtgccgcatctgcatgcgcaacttcagccgcagcgaccacctcaccacccacatccgcacccacacaggcgaaaagcccttcgcctgcgacatctgtggaagaaagtttgccaggagcgatgaacgcaagaggcataccaagatccacttgcggcagaaggacaagaaagcagacaaaagtgttgtggcctcttcggccacctcctctctctcttcctacccgtccccggttgctacctcttacccgtccccggttactacctcttatccatccccggccaccacctcatacccatcccctgtgcccacctccttctcctctcccggctcctcgacctacccatcccctgtgcacagtggcttcccctccccgtcggtggccaccacgtactcctctgttccccctgctttcccggcccaggtcagcagcttcccttcctcagctgtcaccaactccttcagcgcctccacagggctttcggacatgacagcaaccttttctcccaggacaattgaaatttgctaaagggaaaggggaaagaaagggaaaagggagaaaaagaaacacaagagacttaaaggacaggaggaggagatggccataggagaggagggttcctcttaggtcagatggaggttctcagagccaagtcctccctctctactggagtggaaggtctattggccaacaatcctttctgcccacttccccttccccaattactattccctttgacttcagctgcctgaaacagccatgtccaagttcttcacctctatccaaagaacttgatttgcatggattttggataaatcatttcagtatcatctccatcatatgcctgaccccttgctcccttcaatgctagaaaatcgagttggcaaaatggggtttgggcccctcagagccctgccctgcacccttgtacagtgtctgtgccatggatttcgtttttcttggggtactcttgatgtgaagataatttgcatattctattgtattatttggagttaggtcctcacttgggggaaaaaaaaaaaagaaaagccaagcaaaccaatggtgatcctctattttgtgatgatgctgtgacaataagtttgaacctttttttttgaaacagcagtcccagtattctcagagcatgtgtcagagtgttgttccgttaacctttttgtaaatactgcttgaccgtactctcacatgtggcaaaatatggtttggtttttctttttttttttttttgaaagtgttttttcttcgtccttttggtttaaaaagtttcacgtcttggtgccttttgtgtgatgcgccttgctgatggcttgacatgtgcaattgtgagggacatgctcacctctagccttaaggggggcagggagtgatgatttgggggaggctttgggagcaaaataaggaagagggctgagctgagcttcggttctccagaatgtaagaaaacaaaatctaaaacaaaatctgaactctcaaaagtctatttttttaactgaaaatgtaaatttataaatatattcaggagttggaatgttgtagttacctactgagtaggcggcgatttttgtatgttatgaacatgcagttcattattttgtggttctattttactttgtacttgtgtttgcttaaacaaagtgactgtttggcttataaacacattgaatgcgctttattgcccatgggatatgtggtgtatatccttccaaaaaattaaaacgaaaataaagtagctgcgattggg

SEQ ID NO: 4 - DNA, 5989 bp, Homo sapiens
gcgtcccgggtccccgcgccgcgccgcgacctgcagaccccgccgccgcgctcgggcccgtctcccacgcccccgccgccccgcgcgcccaactccgccggccgccccgccccgccccgcgcgctccagacccccggggcggctgccgggagagatgctggaagaaacttcttaaatgaccgcgtctggctggccgtggagcctttctgggttggggagaggaaaggaaagtggaaaaaacctgagaacttcctgatctctctcgctgtgagacatgtctgagactcctgctcagtgtagcattaagcaggaacgaatttcatatacacctccagagagcccagtgccgagttacgcttcctcgacgccacttcatgttccagtgcctcgagcgctcaggatggaggaagactcgatccgcctgcctgcgcacctgcgcttgcagccaatttactggagcagggatgacgtagcccagtggctcaagtgggctgaaaatgagttttctttaaggccaattgacagcaacacgtttgaaatgaatggcaaagctctcctgctgctgaccaaagaggactttcgctatcgatctcctcattcaggtgatgtgctctatgaactccttcagcatattctgaagcagaggaaacctcggattcttttttcaccattcttccaccctggaaactctatacacacacagccggaggtcatactgcatcagaaccatgaagaagataactgtgtccagaggacccccaggccatccgtggataatgtgcaccataaccctcccaccattgaactgttgcaccgctccaggtcacctatcacgacaaatcaccggccttctcctgaccccgagcagcggcccctccggtcccccctggacaacatgatccgccgcctctccccggctgagagagctcagggacccaggccgcaccaggagaacaaccaccaggagtcctaccctctgtcagtgtctcccatggagaataatcactgcccagcgtcctccgagtcccacccgaagccatccagcccccggcaggagagcacacgcgtgatccagctgatgcccagccccatcatgcaccctctgatcctgaacccccggcactccgtggatttcaaacagtccaggctctccgaggacgggctgcatagggaagggaagcccatcaacctctctcatcgggaagacctggcttacatgaaccacatcatggtctctgtctccccgcctgaagagcacgccatgcccattgggagaatagcagactgtagactgctttgggattacgtctatcagttgctttctgacagccggtacgaaaacttcatccgatgggaggacaaagaatccaaaatattccggatagtggatcccaacggactggctcgactgtggggaaaccataagaacagaacaaacatgacctatgagaaaatgtccagagccctgcgccactactacaaactaaacattatcaggaaggagccaggacaaaggcttttgttcaggtttatgaaaaccccagatgaaatcatgagtggccgaacagaccgtctggagcacctagagtcccaggagctggatgaacaaatataccaagaagatgaatgctgaaggaaccaacagtccacctcagcgggccagcagcccagggaacccctgcccaccaggattgctggaagtgtgacggagcaggcgggctgaggagagtggaaaaggaagcgacccagaaatggcagggacacttctcttgcagaccaagagggaccctggagcaccttagacaaactacccagcacaggcggggctggaattctggcggagggcatgagcctgggactccatgtcacgtttccttctgatttggaatctctccatctgtaattcctcaccctcacccttccaccgttgttagtatcatggtgtttttgtttttgtttttgttttaagaacctgcagtttgactcttcatcgttcatctaggggaagacatctgatgttgttttcctatggaaatatatatctattatatatatattttttgcaaatctcacaaagtgcggcaagcccagctggtcaggaaagagaatacttgcagaggggttcaggttcctctttttcctgccacgtggatcaggtctgttcctgttactgttgggtcttggctgaaaaaaaaaaatgcttttaaaaaagataaaatgaaaaggagagctctctttttctctctcttgctctgttcttcccttggtcccctctgtcctcccgccctgcctgcagttgagattcagatgccttctgacagagttcagcctcttggagagtcttggggattgttggcacctaaacagaatcagtgacccgggtgctttgtggccagcagcacagaatcaaacccgcatcccagcattgggccacccatctgagggaggccaaaatcatcacagatgctgctgtgctgcagacagatacatgctagtccagagagccgcccctgagatggctgtgagaaccatgtgtctaaggcgtaagataaggatggaaggctgtccaagttatttggaaggcctcggcagcttgggattagcttgggagcgcagcgctgcaaagtggaaaatatgaaaagaccacacaggcccagcagtccagaaactgggcaaaaatattctgcagtggggatttatttttccaaagcaggtaacagaggctagtgagaaagaaaagctcctctctgctccattccaaaggccatcttgtggtcagtttcatgccctcacctgatttttttttttttttttttttttcaattcctaaccttttttaaagtttcctggtctccactggacacagagctttggagacggaggatcccagagggcagtctcagttgcaatcagtgtgtgcccagcctgggcagacaggaaattcctcggatacattattttttctttctttcatagctgtgtctcagaaaggacccatttgtggctctttttcacctcaaaataagatcgatggtatcttgtaaaatgagggtagtgccacttcttagtatttttgaaagctgttttagattttttttttttttccttttctagccatctaaattgactcttccaatataggtctcagaaatccaatatttggagtacaatttcttttaatccagattacacctgccttacaaagcaccccctccttgttcccctctgtttcctctactcagttgggggagaaactcacagctcctccgggatacatatgtgccctcagcagcagctcccaggtgaagttaccagacccctgggcttctccccagctttttctgagttgagtcagacatgtagagtttgggtcacacaggcaagaggaattttccctcggccttactgacaaggacaccaacctagggtgcaaacagatggactatggttcaaggacactggaattgaggagctgatcaaggctctcttcagccttgctctgtccctgcctcttatcagagcacaggtagacacacgggcatagccagcccactcctactgtcacaggcgccccaccattcaaccttccgggaggtcagggaccttctatatgaggcgagtgggtctcagtctgcttgaatggtgatgagattctgctggatctcagcacgctgcaggtgtcttttgagagcattcagtaggacatggtgatccctatttcagcctctaagatgactggtattctatctgaaatgcagagattaagccaaatacctgatgtattgtgaaagccactgattttaagaatggagagaaagggattttttactgcatccctctgtatgaatatgaaatcagagaccagggcatgatgttgctaggattagagcctctcagtctggcctcttcacccaagtgcaagaactcagtctcttactgttcaaagaatcttaacagttgaattatggagggaaattcccttttgccccaagcatttctatatttaaagcaatatcccaggagaatatgttagacttaggatgataccttcagccacttgaagaagaaatagaaggcgctcattccaatatagtctttatttcccattcagatacaggttgagcatccctaatctgaacagttaaaacccccaaatgccccaaaatccaaaccttcctgaacgctatgacaccatgagtggaaaattccacacctaacaaacacatttgctttcttatggttcaatgtacacaaactgttttatatagaaaatgatttcaaatatcataaaattaccttcaggctatgtgtataaagtatatatgagccataaatgaattttgtgtttagactttgtgtccatccccaagatctctcattttatatatatatatatatatatatatatatatatatatatatatatatacacacacacacacatacacaaatattccaggatacaaaaaaaaacatttaaaaatccgagacccagaacacttctggtcccaagcatttcagataagggatatcaatctgtactaccaataaggatttcgtaattcccctaactgcaaatgtcctcttcatttgttctttatgagaaaacccgggtagtgccagcacctggatacagtatttacaccctgcagaccctaaagatttcagattcagttagcaaaccttgatgaagcacctgctggacactgagggacccaaagctcaatcagccataatccctgctttcagagtttatattgtacctgcctaatccacccggcgtgactcatttcaacactaagtactaggggtgttgtcaggagacaaatctgaagtcaggagaggaaaatgcaaaggagccctgccgtgtgatggatgtgcattctcacttgggtcttgaagttctcattcctacatctcaagctagccaggcagtctcctctctatcagaagaaagcactggtaattggctagactggctatgttgaaggtaacatgaactctaagatcttgacccagggcgacttggttttgcttaaggtggcatcaccaatgttccaaatcctttagggagatgagggtatccccacagaaaaagaggaataatagaccaatggattttctcctttcaccagtatgtttggaaccctctgatccaatgtcttttgatactgatctcttgtccaaatgagaatgtcgctttagctgaaattcaaatggctgtgacaatttaccgaaatgatgaagtaaccaccattcccacctttcactgcctaggctccaagtctgaatacatttttgaaataggaactcccttttgcaaaaaagaaacctgggtgtcagggaggtgaagtgacttgccctaggagcagacagcatgccaagaatggaattaggctcaggatccagcctgggctcaccctgtgtggctcattcccacccaggaaactgaagataaaagatttgggaaaacacaccaagaaaaaggggcagttttctttgcccaagcatttggtgctagttagaggctgttcactctctcctgctcctcttcggagtagaaataaaggctgtgacacaaggaagccagtggggtgggagggaggcaccataatccctccctaaaacccacagaagactaacctgatactcttttgacccaactgcatcaacactaaacagctgcagaccccctgaatctttcacacatgccaagtgaacattcttgatgatttctctttgtgaccgcaaccacctgcaaaccagaacgactctagaatttccttccccgcccccctttttgtttagtttctaatctcttgtttatgaggtgtggggtttataagggactgaatcaaatgaatgtaacaaaaaagaaaaaaaaaacaaaaaaaaatgccttttctcagggccagtgagttgcaaataatttttaaagaaaagcctataattacatcatctcaataaattttttataaaaaaaaaaaaa

SEQ ID NO: 5 - DNA, 4891 bp, Homo sapiens
agccttctttcctcctcgcccgcagtctcgcggagccctgctgcttatctacgttgctaagccgggcgatttccttgttcctcctgcgaaacggtgcggtctggacacgtctccggggtgggtcgtccggccttcgatcttagacgaattttacaatgtgaagttctgcatagatgccagtcaaccagatgttggaagctggctcaagtacattagattcgctggctgttatgatcagcacaaccttgttgcatgccagataaatgatcagatattctatagagtagttgcagacattgcgccgggagaggagcttctgctgttcatgaagagcgaagactatccccatgaaactatggcgccggatatccacgaagaacggcaatatcgctgcgaagactgtgaccagctctttgaatctaaggctgaactagcagatcaccaaaagtttccatgcagtactcctcactcagcattttcaatggttgaagaggactttcagcaaaaactcgaaagcgagaatgatctccaagagatacacacgatccaggagtgtaaggaatgtgaccaagtttttcctgatttgcaaagcctggagaaacacatgctgtcacatactgaagagagggaatacaagtgtgatcagtgtcccaaggcatttaactggaagtccaatttaattcgccaccagatgtcacatgacagtggaaagcactatgaatgtgaaaactgtgccaaggttttcacggaccctagcaaccttcagcggcacattcgctctcagcatgtcggtgcccgggcccatgcatgcccggagtgtggcaaaacgtttgccacttcgtcgggcctcaaacaacacaagcacatccacagcagtgtgaagccctttatctgtgaggtctgccataaatcctatactcagttttcaaacctttgccgtcataagcgcatgcatgctgattgcagaacccaaatcaagtgcaaagactgtggacaaatgttcagcactacgtcttccttaaataaacacaggaggttttgtgagggcaagaaccattttgcggcaggtggattttttggccaaggcatttcacttcctggaaccccagctatggataaaacgtccatggttaatatgagtcatgccaacccgggccttgctgactattttggcgccaataggcatcctgctggtcttacctttccaacagctcctggattttcttttagcttccctggtctgtttccttccggcttgtaccacaggcctcctttgatacctgctagttctcctgttaaaggactatcaagtactgaacagacaaacaaaagtcaaagtcccctcatgacacatcctcagatactgccagctacacaggatattttgaaggcactatctaaacacccatctgtaggggacaataagccagtggagctccagcccgagaggtcctctgaagagaggccctttgagaaaatcagtgaccagtcagagagtagtgaccttgatgatgtcagtacaccaagtggcagtgacctggaaacaacctcgggctctgatctggaaagtgacattgaaagtgataaagagaaatttaaagaaaatggtaaaatgttcaaagacaaagtaagccctcttcagaatctggcttcaataaataataagaaagaatacagcaatcattccattttctcaccatctttagaggagcagactgcggtgtcaggagctgtgaatgattctataaaggctattgcttctattgctgaaaaatactttggttcaacaggactggtggggctgcaagacaaaaaagttggagctttaccttacccttccatgtttcccctcccattttttccagcattctctcaatcaatgtacccatttcctgatagagacttgagatcgttacctttgaaaatggaaccccaatcaccaggtgaagtaaagaaactgcagaagggcagctctgagtccccctttgatctcaccactaagcgaaaggatgagaagcccttgactccagtcccctccaagcctccagtgacacctgccacaagccaagaccagcccctggatctaagtatgggcagtaggagtagagccagtgggacaaagctgactgagcctcgaaaaaaccacgtgtttgggggaaaaaaaggaagcaacgtcgaatcaagacctgcttcagatggttccttgcagcatgcaagacccactcctttctttatggaccctatttacagagtagagaaaagaaaactaactgacccacttgaagctttaaaagagaaatacttgaggccttctccaggattcttgtttcacccacaattccaactgcctgatcagagaacttggatgtcagctattgaaaacatggcagaaaagctagagagcttcagtgccctgaaacctgaggccagtgagctcttacagtcagtgccctctatgttcaacttcagggcgcctcccaatgccctgccagagaaccttctgcggaagggaaaggagcgctatacctgcagatactgtggcaagatttttccaaggtctgcaaacctaacacggcacttgagaacccacacaggagagcagccttacagatgcaaatactgtgacagatcatttagcatatcttctaacttgcaaaggcatgttcgcaacatccacaataaagagaagccatttaagtgtcacttatgtgataggtgttttggtcaacaaaccaatttagacagacacctaaagaaacatgagaatgggaacatgtccggtacagcaacatcgtcgcctcattctgaactggaaagtacaggtgcgattctggatgacaaagaagatgcttacttcacagaaattcgaaatttcattgggaacagcaaccatggcagccaatctcccaggaatgtggaggagagaatgaatggcagtcattttaaagatgaaaaggctttggtgaccagtcaaaattcagacttgctggatgatgaagaagttgaagatgaggtgttgttagatgaggaggatgaagacaatgatattactggaaaaacaggaaaggaaccagtgacaagtaatttacatgaaggaaaccctgaggatgactatgaagaaaccagtgccctggagatgagttgcaagacatccccagtgaggtataaagaggaagaatataaaagtggactttctgctctagatcatataaggcacttcacagatagcctcaaaatgaggaaaatggaagataatcaatattctgaagctgagctgtcttcttttagtacttcccatgtgccagaggaacttaagcagccgttacacagaaagtccaaatcgcaggcatatgctatgatgctgtcactgtctgacaaggagtccctccattctacatcccacagttcttccaacgtgtggcacagtatggccagggctgcggcggaatccagtgctatccagtccataagccacgtatgacgttatcaaggttgaccagagtgggaccaagtccaacagtagcatggctctttcatataggactatttacaagactgctgagcagaatgccttataaacctgcagggtcactcatctaaagtctagtgaccttaaactgaatgatttaaaaaagaaaagaaagaaaaaagaaactatttattctcgatattttgttttgcacagcaaaggcagctgctgacttctggaagatcaatcaatgcgacttaaagtgattcagtgaaaacaaaaaacttggtgggctgaaggcatcttccagtttaccccaccttagggtatgggtgggtgagaagggcagttgagatggcagcattgatatgaatgaacactccatagaaactgaattctcttttgtacaagatcacctgacatgattgggaacagttgcttttaattacagatttaatttttttcttcgttaaagttttatgtaatttaaccctttgaagacagaagtagttggatgaaatgcacagtcaattattatagaaactgataacagggagtacttgttcccccttttgccttcttaagtacattgtttaaaactagggaaaaagggtatgtgtatattgtaaactatggatgttaacactcaaagaggttaagtcagtgaagtaacctattcatcaccagtaccgctgtaccactaataaattgtttgccaaatccttgtaataacatcttaattttagacaatcatgtcactgtttttaatgtttatttttttgtgtgtgttgcgtgtatcatgtatttatttgttggcaaactattgtttgttgattaaaatagcactgttccagtcagccactactttatgacgtctgaggcacacccctttccgaatttcaaggaccaaggtgacccgacctgtgtatgagagtgccaaatggtgtttggcttttcttaacattcctttttgtttgtttgttttgttttccttcttaatgaactaaatacgaatagatgcaacttagtttttgtaatactgaaatcgattcaattgtataaacgattataatttctttcatggaagcatgattcttctgattaaaaactgtactccatattttatgctggttgtctgcaagcttgtgcgatgttatgttcatgttaatcctatttgtaaaatgaagtgttcccaaccttatgttaaaagagagaagtaaataacagactgtattcagttattttgccctttattgaggaaccagatttgttttctttttgtttgtaatctcattttgaaataatcagcaagttgaggtactttcttcaaatgctttgtacaatataaactgttatgcctttcagtgcattactatgggaggagcaactaaaaaataaagacttacaaaaaggagtattttt

SEQ ID NO: 6 - DNA, 3383 bp, Homo sapiens
gtgagcgccaggaaggtagcgaggccagcgtcgccccgggactcgctgctcaagtctgtctattgcctgccgccacatccatcctagcagggccccgtcgcccaccaggcggacaaaagcggtccgctgaacaccatgcggccgctcggcgtgccgcccaggctctgctggtgagcgccgccaccccgcgcccaggtcccgcgagcccgcctgccgcgcacctcgccctgctcccagctctactccaggccccgtccgcccgggggcgccgcccaccgcgcctcgctcgggccgttgccgtctgcacccagaccctgagccgccgccgccggccatggaggtggcgcccgagcagccgcgctggatggcgcacccggccgtgctgaatgcgcagcaccccgactcacaccacccgggcctggcgcacaactacatggaacccgcgcagctgctgcctccagacgaggtggacgtcttcttcaatcacctcgactcgcagggcaacccctactatgccaaccccgctcacgcgcgggcgcgcgtctcctacagccccgcgcacgcccgcctgaccggaggccagatgtgccgcccacacttgttgcacagcccgggtttgccctggctggacgggggcaaagcagccctctctgccgctgcggcccaccaccacaacccctggaccgtgagccccttctccaagacgccactgcacccctcagctgctggaggccctggaggcccactctctgtgtacccaggggctgggggtgggagcgggggaggcagcgggagctcagtggcctccctcacccctacagcagcccactctggctcccaccttttcggcttcccacccacgccacccaaagaagtgtctcctgaccctagcaccacgggggctgcgtctccagcctcatcttccgcggggggtagtgcagcccgaggagaggacaaggacggcgtcaagtaccaggtgtcactgacggagagcatgaagatggaaagtggcagtcccctgcgcccaggcctagctactatgggcacccagcctgctacacaccaccccatccccacctacccctcctatgtgccggcggctgcccacgactacagcagcggactcttccaccccggaggcttcctggggggaccggcctccagcttcacccctaagcagcgcagcaaggctcgttcctgttcagaaggccgggagtgtgtcaactgtggggccacagccacccctctctggcggcgggacggcaccggccactacctgtgcaatgcctgtggcctctaccacaagatgaatgggcagaaccgaccactcatcaagcccaagcgaagactgtcggccgccagaagagccggcacctgttgtgcaaattgtcagacgacaaccaccaccttatggcgccgaaacgccaacggggaccctgtctgcaacgcctgtggcctctactacaagctgcacaatgttaacaggccactgaccatgaagaaggaagggatccagactcggaaccggaagatgtccaacaagtccaagaagagcaagaaaggggcggagtgcttcgaggagctgtcaaagtgcatgcaggagaagtcatcccccttcagtgcagctgccctggctggacacatggcacctgtgggccacctcccgcccttcagccactccggacacatcctgcccactccgacgcccatccacccctcctccagcctctccttcggccacccccacccgtccagcatggtgaccgccatgggctagggaacagatggacgtcgaggaccgggcactcccgggatgggtggaccaaacccttagcagcccagcatttcccgaaggccgacaccactcctgccagcccggctcggcccagcaccccctctcctggagggcgcccagcagcctgccagcagttactgtgaatgttccccaccgctgagaggctgcctccgcacctgaccgctgcccaggtggggtttcctgcatggacagttgtttggagaacaacaaggacaactttatgtagagaaaaggaggggacgggacagacgaaggcaaccatttttagaaggaaaaaggattaggcaaaaataatttattttgctcttgtttctaacaaggacttggagacttggtggtctgagctgtcccaagtcctccggttcttcctcgggattggcgggtccacttgccagggctctgggggcagatttgtggggacctcagcctgcaccctcttctcctctggcttccctctctgaaatagccgaactccaggctgggctgagccaaagccagagtggccacggcccagggagggtgagctggtgcctgctttgacgggccaggccctggagggcagagacaatcacgggcggtcctgcacagattcccaggccagggctgggtcacaggaaggaaacaacattttcttgaaaggggaaacgtctcccagatcgctcccttggctttgaggccgaagctgctgtgactgtgtccccttactgagcgcaagccacagcctgtcttgtcaggtggaccctgtaaatacatcctttttctgctaacccttcaaccccctcgcctcctactctgagacaaaagaaaaaatattaaaaaaatgcataggcttaactcgctgatgagttaattgttttatttttaaactctttttgggtccagttgattgtacgtagccacaggagccctgctatgaaaggaataaaacctacacacaaggttggagctttgcaattctttttggaaaagagctgggatcccacagccctagtatgaaagctgggggtggggaggggcctttgctgcccttggtttctgggggctggttggcatttgctggcctggcagggggtgaaggcaggagttgggggcaggtcaggaccaggacccagggagaggctgtgtccctgctggggtctcaggtccagctttactgtggctgtctggatccttcccaaggtacagctgtatataaacgtgtcccgagcttagattctgtatgcggtgacggcggggtgtggtggcctgtgaggggcccctggcccaggaggaggattgtgctgatgtagtgaccaagtgcaatatgggcgggcagtcgctgcagggagcaccacggccagaagtaacttattttgtactagtgtccgcataagaaaaagaatcggcagtattttctgtttttatgttttatttggcttgttttattttggattagtgaactaagttattgttaattatgtacaacatttatatattgtctgtaaaaaatgtatgctatcctcttattcctttaaagtgagtactgttaagaataataaaatactttttgtgaatgcccaaaaaaaaa

SEQ ID NO: 7 - DNA, 1663 bp, Homo sapiens
aaaaaggagaagtatctatttgtgcaaagagtcacacagttgacagagtggaggccagtcccgagagaggctttgcagttcccacctcgggaagctccggcagaacccaggcgagggacagctccggacaggtgtggggtgcacactgaaaatgccacgctccttcctggtgaagagcaagaaggctcacacctaccaccagccccgtgtgcaggaagatgaaccgctctggcctcctgcccttaccccggtgcccagagaccaggctccaagcaacagccctgtccttagcactctattcccaaaccagtgcctggactggaccaacctcaaacgagagccggagctggagcaggaccagaacttggccaggatggccccggcaccagagggccccattgtgctgtcccgaccccaggatggggactctccactgtccgactcacccccattctacaagcctagcttctcctgggacaccttggccacaacctatggccacagctaccggcaggccccctccaccatgcagtcagccttcctggagcactccgtcagcctgtacggcagtcctcttgtgcccagcactgagcccgccttggacttcagcctccgctactccccaggcatggatgcgtaccactgtgtgaagtgcaacaaggagcgcagcttcgagtgccgcatgtgcggcaaggccttcaagcgctcgtccacgctgtccacccacctgctcatccactcagacacgcggccctacccctgccagttctgcggcaagcgtttccaccagaagtccgacatgaagaagcacacctacatccacacaggtgagaagccgcacaagtgccaggtgtgcggaaaggccttcagccagagctccaacctcatcacccacagccgcaagcacacaggcttcaagcccttcagctgtgagctgtgcaccaaaggcttccagcgcaaggtggacctgcggcggcaccgcgagagccagcacaatctcaagtgaggctgcgccggctcccagctcctggccagcctgccctgcggtcctgtcacctggaggccagcctcacatgcccaaatctccagtctcctggaggtgggactggacaggagtctaccagcttgttttgagactcatgaaattgctgtgtgaccttgggcaagtcacttaccctgtctggatcaacatttctcctgctgccaagtgtgggagcctggctgggtctttctcagcagaagttgtttccaggtgtgctcaagtgccttcctctagcagagcacagaaagctagaatacccccagggagacagggatgccaagagtagaccagagctgggacccacagacagaacctccacctgcctgctgcccactgagctgggacctggtcaccttggattttagccggcctctttctggctataacaggcagagtcggagctgcctcccaccccagtcagaagcctggcaccccctctgcttcggccagatgtgctggctgactccgacttccgaccagcactcagctggcctctggggattctagctccacaaccaggccgtgaggctggagaaactggcagttattgctgtcaaaagcctgttctctcaactgctgtcaataaaattaaagatacagatttgctgccaaaaaaaaaaa

SEQ ID NO: 8 - DNA, 3705 bp, Homo sapiens
tgaagaccagctgggagcccactgcctgctgccacctccaactccggccccctcaccatgcactccctggacgagccgctcgacctgaagctgagtatcaccaagctccgggcggcaagagagaagcgggagaggacgctgggtgtggtccggccccgtgctctgcacagggagctgggcctggtggatgacagccccacacctggctctccaggctccccgccctcaggcttcctgctgaactccaagttccccgagaaggtggagggacgcttttcagcagcccctctcgtggacctcagcctgtcaccaccatctgggctggactcccccaatggcagcagctcgctgtcccccgagcgccagggcaacggggacctgcctccagtgcccagtgcctcggacttccagccactgcgctatttggatggtgtccccagctccttccagttcttcctgcccctcggctccgggggggccctgcacctgcctgcctcctccttccttacccctcccaaggacaagtgcctctcgccagacctgcccctgcccaagcagctggtgtgtcgctgggccaagtgtaaccagctctttgagctcctgcaagacctggtggaccatgtcaacgattaccatgtcaagcccgagaaggatgcggggtactgctgccactgggagggctgcgcccgccatggccgaggtttcaacgccaggtacaagatgctcatccacatccgcacacacaccaacgagaagccacaccgctgtccgacctgcagcaagagcttctcccgcctggagaacctgaagatccacaaccggtcgcacacaggtgagaagccctacgtctgcccctacgagggctgcaacaagcgctattccaactccagtgaccgctttaagcacacgcgcacccactatgtggacaagccctactactgcaagatgcccggctgccacaagcgctacacggaccccagctcactgcgcaagcacatcaaggcccatggccactttgtgtcccacgagcagcaagagctcctgcagctgcgcccaccccccaagccgccactgcccgcccccgacggcggcccctatgtcagtggggcccagatcatcatccccaacccagctgccctctttggaggccctggcctgcccggcttacccctacccctggcccccggcccccttgacctcagtgccctggcctgtggcaacggtgggggcagtgggggtggggggggcatgggccctgggctgccaggccccgtcctgcctctcaatctggccaagaacccgctgctgccctcgccctttggggctggcggactgggcttgcctgtggtctccctccttgctggcgcagctggtggcaaggccgagggggagaaggggcgtgggtcggtgcccaccagggccctgggcatggagggccacaagacgccccttgaaaggacggagagcagctgctcccggccaagccccgatggactccccctgctgccaggcaccgtgctggacctgtccacgggcgtcaactcagctgccagcagcccagaggcgttggcccctggctgggtggtcatcccgccgggctcggtgctgctcaaaccggctgtggtgaactgagcccatcctgcggacagttgtggtgcccccccggcagctcccggcactgcccccgacgaacggaaactcttctgtgaaatagcaataatgtcctactgcccgggcagccccagcccagcccgccgggagcaaggatggtgctaggtcattcatggctggcctcccagcccccgggtggggacctggcctgtcatgcagggagagctgtgctcctgggtgctgaagcctcgctcctgtctgtcccccaccacctggccctcagcttctgagaggctttcccctgcccgacctcctcccgtttccctctcccaccctggcacctccctcacctagtgaccacccatggcaagttgccctctcccagcagagggggtgggtggggtggcatctgccctccctgctagcaccaggctcccccttcctgagaggagcccccagggaccagaggcctgcccttccctcctaggcttacccagcccctgccctgggggctccttggacccctttccctctgaccctgcctccagagggaaagcaagacagatgcaggcccctgcaaagccccaggtagaagcatgccccccaggacaaggcgcctcccactagttaggaggaggcccgctctgcagccgccgtcctcaccccaggccaggcctgcagtaccagacgggatagctggccactccacccctgcaccccagggtctcctccctctaccttttggggcaccctgggagcgtgggaagcaggtccgagggcccctgagctggcaaggggaggtgccaggccagctgtggtgccaagatactgagtgacctgggccctggctcagggagcatgtggggccaggcccagcgccccgtcttcctccttctacccccgctgggcctggcctgggcagcgccccctgcagaggcctttgggtccttggtcctgtaacaggaagggggaggctggctggggacgaccgaccacaggctgggacacagctcctggtctgggggctccaagtgacagcatgcaggggagggggctcccagtcagtgctgtgttgggagctttctggaggctgtggactgaaggccttgagggaagcagtggctggaggagggtgctggacccatgacacgttgcttcctctggcttttccctgctgggccgctttctcagaggcacttccccacccctaacacccagtgggcccccccaggttctgtgccactcagagggaccctggcaggggccagaaccacttaagggtggtgctggagggccttgtgccccagtcccatcccaggacgccctgagggatggacgcagccatgcaccccccatctggggcctctccctgctccctctcccacctggcagctgggagttctggcttctaggcctgccctgtcaccaggcctctgagtggccaggcccttccacctccccatctgtaaaacgaggcagctgcccggacagccttggggtccttagtggccctgcaggtcctctggcagctctgctgaccccaccctctcccggactgcccttctgtcccagaggggtcaccctgacccggcccaccttgccactgggctttggactccagccctgacagggcccagccacactggctctgcccctcgaaggggctatgagcaaggtaggagggagctggtctcctttcttcgggccccacccaggccctgagcaccccccacccctgtgagggccccaggccttaagtccctggcggggtcatgggtttgcgacttgagcagagcggaggaacagggcactggaaggccgacgagctcagcatgcgactcggtgacggaccaggctcggcagggccggtgtactttttgtggttgtcattggtgtgttgttgcacattccaggacgtcagtattttaacaggttctaagtgcctttctatcgtagcttatgttttcctcctcttggctccattgctgttagcatagagttttaaaaaaagagataagctaatgactataacaatatattcctccatgggagaggaagtttataaagaaacaataaaagtgagttgcaaagatg

SEQ ID NO: 9 - DNA, 5607 bp, Homo sapiens
actcttgtcagggccgcggcacatgggcggccggatgcgctgagcccggcgctgcggggccgcggagcgctggggagcagcggccgccggcgcggggaggggggtggggtgggacggcgcaccgcctccggtgctggcactaggggctggggtcggcgcggtgtcttctgcccttctgcagccgtcgacatttttttttctttctttttttcaattttgaacattttgcaaaacgaggggttcgaggcaggtgagagcatcctgcacgtcgccggggagcccgcgggcacttggcgcgctctcctgggaccgtctgcactggaaacccgaaagtttttttttaatatatatttttatgcagatgtatttataaagatataagtaatttttttcttcccttttctccaccgccttgagagcgagtacttttggcaaaggacggaggaaaagctcagcaacattttagggggcggttgtttctttcttatttctttttttaaggggaaaaaatttgagtgcatcgcgatggagaaaatgtcccgaccgctccccctgaatcccacctttatcccgcctccctacggcgtgctcaggtccctgctggagaacccgctgaagctcccccttcaccacgaagacgcatttagtaaagataaagacaaggaaaagaagctggatgatgagagtaacagcccgacggtcccccagtcggcattcctggggcctaccttatgggacaaaacccttccctatgacggagatactttccagttggaatacatggacctggaggagtttttgtcagaaaatggcattccccccagcccatctcagcatgaccacagccctcaccctcctgggctgcagccagcttcctcggctgccccctcggtcatggacctcagcagccgggcctctgcaccccttcaccctggcatcccatctccgaactgtatgcagagccccatcagaccaggtcagctgttgccagcaaaccgcaatacaccaagtcccattgatcctgacaccatccaggtcccagtgggttatgagccagacccagcagatcttgccctttccagcatccctggccaggaaatgtttgaccctcgcaaacgcaagttctctgaggaagaactgaagccacagcccatgatcaagaaagctcgcaaagtcttcatccctgatgacctgaaggatgacaagtactgggcaaggcgcagaaagaacaacatggcagccaagcgctcccgcgacgcccggaggctgaaagagaaccagatcgccatccgggcctcgttcctggagaaggagaactcggccctccgccaggaggtggctgacttgaggaaggagctgggcaaatgcaagaacatacttgccaagtatgaggccaggcacgggcccctgtaggatggcatttttgcaggctggctttggaatagatggacagtttgtttcctgtctgatagcaccacacgcaaaccaacctttctgacatcagcactttaccagaggcataaacacaactgactcccattttggtgtgcatctgtgtgtgtgtgcgtgtatatgtgcttgtgctcatgtgtgtggtcagcggtatgtgcgtgtgcgtgttcctttgctcttgccattttaaggtagccctctcatcgtcttttagttccaacaaagaaaggtgccatgtctttactagactgaggagccctctcgcgggtctcccatcccctccctccttcactcctgcctcctcagctttgcttcatgttcgagcttacctactcttccaggactctctgcttggattcactaaaaagggccctggtaaaatagtggatctcagtttttaagagtacaagctcttgtttctgtttagtccgtaagttaccatgctaatgaggtgcacacaataacttagcactactccgcagctctagtcctttataagttgctttcctcttactttcagttttggtgataatcgtcttcaaattaaagtgctgtttagatttattagatcccatatttacttactgctatctactaagtttccttttaattctaccaaccccagataagtaagagtactattaatagaacacagagtgtgtttttgcactgtctgtacctaaagcaataatcctattgtacgctagagcatgctgcctgagtattactagtggacgtaggatattttccctacctaagaatttcactgtcttttaaaaaacaaaaagtaaagtaatgcatttgagcatggccagactattccctaggacaaggaagcagagggaaatgggaggtctaaggatgaggggttaatttatcagtacatgagccaaaaactgcgtcttggattagcctttgacattgatgtgttcggttttgttgttccccttccctcacaccctgcctcgcccccacttttctagttaactttttccatatccctcttgacattcaaaacagttacttaagattcagttttcccactttttggtaatatatatatttttgtgaattatactttgttgtttttaaaaagaaaatcagttgattaagttaataagttgatgttttctaaggccctttttcctagtggtgtcatttttgaatgcctcataaattaatgattctgaagcttatgtttcttattctctgtttgcttttgaacgtatgtgctcttataaagtggacttctgaaaaatgaatgtaaaagacactggtgtatctcagaaggggatggtgttgtcacaaactgtggttaatccaatcaatttaaatgtttactatagaccaaaaggagagattattaaatcgtttaatgtttatacagagtaattataggaagttcttttttgtacagtatttttcagatataaatactgacaatgtattttggaagacatatattatatatagaaaagaggagaggaaaactattccatgttttaaaattatatagcaaagatatatattcaccaatgttgtacagagaagaagtgcttgggggtttttgaagtctttaatattttaagccctatcactgacacatcagcatgttttctgctttaaattaaaattttatgacagtatcgaggcttgtgatgacgaatcctgctctaaaatacacaaggagctttcttgtttcttattaggcctcagaaagaagtcagttaacgtcacccaaaagcacaaaatggattttagtcaaatatttattggatgatacagtgttttttaggaaaagcatctgccacaaaaatgttcacttcgaaattctgagttcctggaatggcacgttgctgccagtgccccagacagttcttttctaccctgcgggcccgcacgttttatgaggttgatatcggtgctatgtgtttggtttataatttgatagatgtttgactttaaagatgattgttcttttgtttcattaagttgtaaaatgtcaagaaattctgctgttacgacaaagaaacattttacgctagattaaaatatcctttcatcaatgggattttctagtttcctgccttcagagtatctaatcctttaatgatctggtggtctcctcgtcaatccatcagcaatgcttctctcatagtgtcatagacttgggaaacccaaccagtaggatatttctacaaggtgttcattttgtcacaagctgtagataacagcaagagatgggggtgtattggaattgcaatacattgttcaggtgaataataaaatcaaaaacttttgcaatcttaagcagagataaataaaagatagcaatatgagacacaggtggacgtagagttggcctttttacaggcaaagaggcgaattgtagaattgttagatggcaatagtcattaaaaacatagaaaaatgatgtctttaagtggagaattgtggaaggattgtaacatggaccatccaaatttatggccgtatcaaatggtagctgaaaaaactatatttgagcactggtctctcttggaattagatgtttatatcaaatgagcatctcaaatgttttctgcagaaaaaaataaaaagattctaataaaatgtattctcttgtgtgccaggagaggtttcagaaacctacctcgtcttacaaatttaaacactttggagtctgtacaggtgccttatatgtaggtcattgtcacgatacacacacacgaacactccctctggactggctgcctctccatccagggcagttaactagcaaacaaggcagatctgcttcatggagcgggaggccatggcttgactctgagtgatttgggtcaaccggagtcagacgcatgtctgcacgctgcagctattatgagagtccctttgtcatttttcaccttttcatcctaagcatctttcagagattaattatttggccattaacaatgaatccaaatcatatcatactgacatcatctagacatgatttggaaggaacagcttaggacctcctgatgaggtcacattgttgtttcttttaactagacttggcaaagaaaggcaaaaattgaccagcctatctttctgctggtgctgccttaaggaggtagtttgttgaggggagggctgtagatcattacttctttctcttcaggaagtggccactttgaaccattcaaataccacattaggcaagactgtgataggccttttgtcttcaaatacaacaggcctccactgacccatccctcaaagcagaaggaccctttgaggagagtacagatgggattccacagtggggtgggtggaatggaaacctgtactagaccacccagaggttccttctaacccactggtttggtggggaactcacagtaattccaaatgtacaatcagatgtctagggtctgttttcggaagaagcaagaattatcagtggcaccctccccactgcccccagtgtaaaacaatagacattctgtgaaatgcaaagctattctttggtttttctagtagtttatctcattttaccctattcttcctttaaggaaaactcaatctttatcacagtcaattagagcgatcccaaggcatgggaccaggcctgcttgcctatgtgtgatggcaattggagatctggatttagcactggggtctcagcaccctgcaggtgtctgagactaagtgatctgccctccaggtggcgatcaccttctgctcctaggtacccccactggcaaggccaaggtctcctccacgttttttctgcaattaataatgtcatttaaaaaatgagcaaagccttatccgaatcggatatagcaactaaagtcaatacattttgcaggaggctaagtgtaagagtgtgtgtgtgtgtgtgtgcgtgcatgtgtgtgtgtgtgtatgtgtgtgaataagtcgacataaagtctttaattttgagcaccttaccaaacataacaataatccattatccttttggcaacaccacaaagatcgcatctgttaaacaggtacaagttgacatgaggttagtttaattgtacaccatgatattggtggtatttatgctgttaagtccaaacctttatctgtctgttattcttaatgttgaataaactttgaattttttcctttcaaaaaaaa

SEQ ID NO: 10 - DNA, 4150 bp, Homo sapiens
cttgaatcttggggcaggaactcagaaaacttccagcccgggcagcgcgcgcttggtgcaagactcaggagctagcagcccgtccccctccgactctccggtgccgccgctgcctgctcccgccaccctaggaggcgcggtgccacccactactctgtcctctgcctgtgctccgtgcccgaccctatcccggcggagtctccccatcctcctttgctttccgactgcccaaggcactttcaatctcaatctcttctctctctctctctctctctctctctctctctctctctctctctctctctctctcgcagggtggggggaagaggaggaggaattctttccccgcctaacatttcaagggacacaattcactccaagtctcttccctttccaagccgcttccgaagtgctcccggtgcccgcaactcctgatcccaacccgcgagaggagcctctgcgacctcaaagcctctcttccttctccctcgcttccctcctcctcttgctacctccacctccaccgccacctccacctccggcacccacccaccgccgccgccgccaccggcagcgcctcctcctctcctcctcctcctcccctcttctctttttggcagccgctggacgtccggtgttgatggtggcagcggcggcagcctaagcaacagcagccctcgcagcccgccagctcgcgctcgccccgccggcgtccccagccctatcacctcatctcccgaaaggtgctgggcagctccggggcggtcgaggcgaagcggctgcagcggcggtagcggcggcgggaggcaggatgagcgcacgcggtgagggcgcggggcagccgtccacttcagcccagggacaacctgccgccccagcgcctcagaagagaggacgcggccgccccaggaagcagcagcaagaaccaaccggtgagccctctcctaagagacccaggggaagacccaaaggcagcaaaaacaagagtccctctaaagcagctcaaaagaaagcagaagccactggagaaaaacggccaagaggcagacctaggaaatggccacaacaagttgttcagaagaagcctgctcaggaggaaactgaagagacatcctcacaagagtctgccgaagaggactagggggcgccaacgttcgatttctacctcagcagcagttggatcttttgaagggagaagacactgcagtgaccacttattctgtattgccatggtctttccactttcatctggggtggggtggggtggggtgggggaggggggggtggggtggggagaaatcacataaccttaaaaaggactatattaatcaccttctttgtaatcccttcacagtcccaggtttagtgaaaaactgctgtaaacacaggggacacagcttaacaatgcaacttttaattactgttttcttttttcttaacctactaatagtttgttgatctgataagcaagagtgggcgggtgagaaaaaccgaattgggtttagtcaatcactgcactgcatgcaaacaagaaacgtgtcacacttgtgacgtcgggcattcatataggaagaacgcggtgtgtaacactgtgtacacctcaaataccaccccaacccactccctgtagtgaatcctctgtttagaacaccaaagataaggactagatactactttctctttttcgtataatcttgtagacacttacttgatgatttttaactttttatttctaaatgagacgaaatgctgatgtatcctttcattcagctaacaaactagaaaaggttatgttcatttttcaaaaagggaagtaagcaaacaaatattgccaactcttctatttatggatatcacacatatcagcaggagtaataaatttactcacagcacttgttttcaggacaacacttcattttcaggaaatctacttcctacagagccaaaatgccatttagcaataaataacacttgtcagcctcagagcatttaaggaaactagacaagtaaaattatcctctttgtaatttaatgaaaaggtacaacagaataatgcatgatgaactcacctaattatgaggtgggaggagcgaaatctaaatttcttttgctatagttatacatcaatttaaaaagcaaaaaaaaaaaaggggggggcaatctctctctgtgtctttctctctctctcttcctctccctctctcttttcattgtgtatcagtttccatgaaagacctgaataccacttacctcaaattaagcatatgtgttacttcaagtaatacgttttgacataagatggttgaccaaggtgcttttcttcggcttgagttcaccatctcttcattcaaactgcacttttagccagagatgcaatatatccccactactcaatactacctctgaatgttacaacgaatttacagtctagtacttattacatgctgctatacacaagcaatgcaagaaaaaaacttactgggtaggtgattctaatcatctgcagttctttttgtacacttaattacagttaaagaagcaatctccttactgtgtttcagcatgactatgtatttttctatgtttttttaattaaaaatttttaaaatacttgtttcagcttctctgctagatttctacattaacttgaaaattttttaaccaagtcgctcctaggttcttaaggataattttcctcaatcacactacacatcacacaagatttgactgtaatatttaaatattaccctccaagtctgtacctcaaatgaattctttaaggagatggactaattgacttgcaaagacctacctccagacttcaaaaggaatgaacttgttacttgcagcattcatttgttttttcaatgtttgaaatagttcaaactgcagctaaccctagtcaaaactatttttgtaaaagacatttgatagaaaggaacacgtttttacatacttttgcaaaataagtaaataataaataaaataaaagccaaccttcaaagaaacttgaagctttgtaggtgagatgcaacaagccctgcttttgcataatgcaatcaaaaatatgtgtttttaagattagttgaatataagaaaatgcttgacaaatattttcatgtattttacacaaatgtgatttttgtaatatgtctcaaccagatttattttaaacgcttcttatgtagagtttttatgcctttctctcctagtgagtgtgctgactttttaacatggtattatcaactgggccaggaggtagtttctcatgacggcttttgtcagtatggcttttagtactgaagccaaatgaaactcaaaaccatctctcttccagctgcttcagggaggtagtttcaaaggccacatacctctctgagactggcagatcgctcactgttgtgaatcaccaaaggagctatggagagaattaaaactcaacattactgttaactgtgcgttaaataagcaaataaacagtggctcataaaaataaaagtcgcattccatatctttggatgggccttttagaaacctcattggccagctcataaaatggaagcaattgctcatgttggccaaacatggtgcaccgagtgatttccatctctggtaaagttacacttttatttcctgtatgttgtacaatcaaaacacactactacctcttaagtcccagtatacctcatttttcatactgaaaaaaaaagcttgtggccaatggaacagtaagaacatcataaaatttttatatatatagtttatttttgtgggagataaattttataggactgttctttgctgttgttggtcgcagctacataagactggacatttaacttttctaccatttctgcaagttaggtatgtttgcaggagaaaagtatcaagacgtttaactgcagttgactttctccctgttcctttgagtgtcttctaactttattctttgttctttatgtagaattgctgtctatgattgtactttgaatcgcttgcttgttgaaaatatttctctagtgtattatcactgtctgttctgcacaataaacataacagcctctgtgatccccatgtgttttgattcctgctctttgttacagttccattaaatgagtaataaagtttggtcaaaacagaaaaaaaaaaa

SEQ ID NO: 11 - DNA, 1657 bp, Homo sapiens
gggtgctatagacgcacaaacgaccgcgagccacaaatcaagcacacatatcaaaaaacaaatgagctcttattttgtaaactcattttgcggtcgctatccaaatggcccggactaccagttgcataattatggagatcatagttccgtgagcgagcaattcagggactcggcgagcatgcactccggcaggtacggctacggctacaatggcatggatctcagcgtcggccgctcgggctccggccactttggctccggagagcgcgcccgcagctacgctgccagcgccagcgcggcgcccgccgagcccaggtacagccagccggccacgtccacgcactctcctcagcccgatccgctgccctgctccgccgtggccccctcgcccggcagcgacagccaccacggcgggaaaaactccctaagcaactccagcggcgcctcggccgacgccggcagcacccacatcagcagcagagagggggttggcacggcgtccggagccgaggaggacgcccctgccagcagcgagcaggcgagtgcgcagagcgagccgagcccggcgccgcccgcccaaccccagatctacccctggatgcgcaagctgcacataagtcatgacaacataggcggcccggaaggcaaaagggcccggacggcctacacgcgctaccagaccctggagctggagaaggagttccacttcaaccgttacctgacccgcagaaggaggattgaaatagcacatgctctttgcctctccgagagacaaattaaaatctggttccaaaaccggagaatgaagtggaaaaaagataataagctgaaaagcatgagcatggccgcggcaggaggggccttccgtccctgagtatctgagcgtttaaagtactgagcagtattagcggatcccgcgtagtgtcagtactaaggtgactttctgaaactcccttgtgttccttctgtgaagaagccctgttctcgttgccctaattcatcttttaatcatgagcctgtttattgccattatagcgcctgtataagtagatctgctttctgttcatctctttgtcctgaatggctttgtcttgaaaaaaaatagatgttttaacttatttatatgaagcaagctgtgttacttgaagtaactataacaaaaaaagaaaagagaaaaaaaaacacacaaaaagtcccccttcaatctcgtttagtgccaatgttgtgtgttgcactcaagttgtttaactgtgcatgtgcgtggaagtgttcctgtctcaatagctccaagctgttaaagatatttttattcaaactacctatattccttgtgtaattaatgctgttgtagaggtgacttgatgagacacaacttgttcgacgtgtagtgactagtgactctgtgatgaaaactgtgactccaagcggtgtgtccctgcgtgcctttataggaccctttgcacgaactctggaagtggctcttataagcgcagcttcagtgatgtatgtttttgtgaacaaagttacaaatattgtccaagtctggctgttttaagcaaactgtgatcagcttttttttttttttttttttttttgtatttgtttttaaggaaaaaatactgactggaacaaaaaataaactttctattgtaagttc

SEQ ID NO: 12 - DNA, 2076 bp, Homo sapiens
agttgttacatgaaatctgcagtttcataatttccgtgggtcgggccgggcgggccaggcgctgggcacggtgatggccaccactggggccctgggcaactactacgtggactcgttcctgctgggcgccgacgccgcggatgagctgagcgttggccgctatgcgccggggaccctgggccagcctccccggcaggcggcgacgctggccgagcaccccgacttcagcccgtgcagcttccagtccaaggcgacggtgtttggcgcctcgtggaacccagtgcacgcggcgggcgccaacgctgtacccgctgcggtgtaccaccaccatcaccaccacccctacgtgcacccccaggcgcccgtggcggcggcggcgccggacggcaggtacatgcgctcctggctggagcccacgcccggtgcgctctccttcgcgggcttgccctccagccggccttatggcattaaacctgaaccgctgtcggccagaaggggtgactgtcccacgcttgacactcacactttgtccctgactgactatgcttgtggttctcctccagttgatagagaaaaacaacccagcgaaggcgccttctctgaaaacaatgctgagaatgagagcggcggagacaagccccccatcgatcccaataacccagcagccaactggcttcatgcgcgctccactcggaaaaagcggtgcccctatacaaaacaccagaccctggaactggagaaagagtttctgttcaacatgtacctcaccagggaccgcaggtacgaggtggctcgactgctcaacctcaccgagaggcaggtcaagatctggttccagaaccgcaggatgaaaatgaagaaaatcaacaaagaccgagcaaaagacgagtgatgccatttgggcttatttagaaaaaagggtaagctagagagaaaaagaaagaactgtccgtcccccttccgccttctcccttctctcacccccaccctagcctccaccatccccgcacaaagcggctctaaacctcaggccacatcttttccaaggcaaaccctgttcaggctggctcgtaggcctgccgctttgatggaggaggtattgtaagctttccattttctataagaaaaaggaaaagttgaggggggggcattagtgctgatagctgtgtgtgttagcttgtatatatatttttaaaaatctacctgttcctgacttaaaacaaaaggaaagaaactacctttttataatgcacaactgttgatggtaggctgtatagtttttagtctgtgtagttaatttaatttgcagtttgtgcggcagattgctctgccaagatacttgaacactgtgttttattgtggtaattatgttttgtgattcaaacttctgtgtactgggtgatgcacccattgtgattgtggaagatagaattcaatttgaactcaggttgtttatgaggggaaaaaaacagttgcatagagtatagctctgtagtggaatatgtcttctgtataactaggctgttaacctatgattgtaaagtagctgtaagaatttcccagtgaaataaaaaaaaattttaagtgttctcggggatgcatagattcatcattttctccaccttaaaaatgcgggcatttaagtctgtccattatctatatagtcctgtcttgtctattgtatatataatctatatgattaaagaaaatatgcataatcagacaagcttgaatattgtttttgcaccagacgaacagtgaggaaattcggagctatacatatgtgcagaaggttactacctagggtttatgcttaattttaattggaggaaatgaatgctgattgtaacggagttaattttattgataataaattatacactatgaaaccgccattgggctactgtagatttgtatccttgatgaatctggggtttccatcagactgaacttacactgtatattttgcaatagttacctcaaggcctactgaccaaattgttgtgttgagatgatatttaactttttgccaaataaaatatattgattcttttctaaaaaaaaaaaaaaaaaaaa

SEQ ID NO: 13 - DNA, 3627 bp, Homo sapiens
ctgggtagggcagggggaaccgacaggccggtgtccccagccgcaaaagagctgctgaactgtccgtttaaatgctgctgggagactcgtaaaaaaatcatcgtggacctggaggatgagaggggcgagctttatttcggtcggattgcggtgtggtggtttagctgcaaggggatgccgcagccccagttgagggggaaaatagttcttaaaaagcatatgcccccctaaggaatgtctctaaagaaccaaatcaaagctgctctttggaaggtatgaatagaatttaaaaaaaaaagatttctatggagcttaaagttcacagccattctgtgtagacaagagctaagaaaaatgtgagaattatacagaaaaccattaatcacttcttttctttaaatacgtatcctctctcctttgttattattcaacagcaaatctccttggaccggctgttgggggaaaaaagtgttagccgtctctcccggatctgcaagggggaaaaaatttggaaccataaagttgaaaacttttttctctcagtttggaagaagcccttcgtcatgaatgggatctgcagagttcgggcgagaggaggcgagaggcgcaaaggaggggagatttgtcgcctgccgctcgctctggggctcgatgtgaatatatattatgtctgcctgttctcccctcgtcggtggctaaggtcagccgcttggaacagaccccggaggaggggggcagagaggggaggtggggggggggggtccggcgtgtcacgtgacccccagggttgccaatgtccggtcctgagggtatcaggcctttccaagttgccacccactgcccaggcctcacccagcgatgcagaaagccacctactacgacaacgccgcggctgctctcttcggaggctattcctcgtaccctggcagcaatggcttcggcttcgatgtccccccccaacccccatttcaggccgccacgcacctggagggcgactaccagcgctcagcttgctcgctgcagtccctgggcaacgctgccccacatgccaagagcaaggagctcaacggcagctgcatgaggccgggtctggcccccgagcccctgtcggccccgcctggctcacccccgcccagtgccgcacctaccagtgccactagcaacagcagtaatgggggcgggcccagcaaaagtggtcccccaaagtgcggtcccggcaccaactccaccctcaccaaacagatattcccctggatgaaagagtcgaggcaaacgtccaagctgaaaaacaactcccccggcacagcagagggctgtggtggcggcggcggtggcggcggcggcggaggcagtggtggcagcgggggcggtggcggcggcggcgggggaggggacaagagccccccggggtcggcggcgtccaagcgggcgcggacggcgtacacgagcgcgcagctggtggagctggagaaggagttccattttaaccgctacctgtgccggcctcgccgtgtagagatggccaacctgctgaacctcagcgagcggcagatcaagatctggttccagaaccggcgcatgaagtacaagaaggaccagaaggccaagggattggcctcgtcgtcggggggcccatctccagccggcagccccccgcagcccatgcagtccacggccggcttcatgaacgccttacactccatgacccccagctacgagagcccgtccccacccgccttcggtaaagcccaccagaatgcctacgcgctgccctccaactaccagccccctctcaaaggctgcggcgccccgcagaagtaccctccgaccccggcgcccgagtatgagccgcacgtcctccaagccaacgggggcgcctacgggacgcccaccatgcagggcagtccggtgtacgtgggcgggggcggctacgcggatccgctgccgccccctgccggcccctccctctatggcctcaaccacctttcccatcacccttccgggaacctggactacaacggggcgccccctatggcgcccagccagcaccacggaccctgcgaaccccaccccacctacacagacctctcctctcaccacgcgcctcctcctcagggtagaatccaagaagcgcccaaattaacacacctgtgatgggaaagggcgaacgaggattaggggatggggaggaagagagagactgtggagctctggggggcaacctggaggtctgaaaagaggagccagagaaggtggtacccaggcttcctggtcagaaccggcctggagctccttcccttccccctggcctgagaggttgcttttaagtcttccaccccttgttccatctgcctgccaacccatcggaaaggaatccacatcatattggagatgaccccatcaaccccagggctccagcactaccaagttggaattccacgcccgggagtggggtagaggaagacgagacaggacgaggcagaaaagcacattttaaaaaccagacaagatggctaggccatcaccaaccaacggacttaccttacatctttgtaggtaattccccccaaatcttgatttttttttttcctcaattatcctttaaaaaataagaaaacacatttcaaacccaaaaggcacaaaacacgttcccttccaactttcccaaaacctcaaatttgttcccatttgaggtttattgaggtacacttctagcccccggtttttctgctctagaacattcatatctatacatcccacccccatcaattacagtttttagagggctcagggatggtgagagatcctgaaagagctgcctatattataaattatatacatttttttttaaggaaaagtgtggaggctagggcaggcaggttgttaggactgaaggtttgcccattctgctgcctccatctcagctccagctccatccccctctccacagaaagcagttggtgacacgaggttctatacttttcttctgttgctctcttgacttaacgtgaaaacagggtatatttgaacaaactgtcccaggcaggggctgggcagggcctgtgtgccttgctcagcctcctgacaggacacttttgttgcacttagaatttacattttaatggatgtaaaaacaactgtgagagatgtctgggcctgcagaagtccagcattgctcaaaaaagcgtgtgttctagtgaacattttcatatatatttattggttatagcctgttaaaatattttcttttttgtattatttatccccctacattatgtatttatatgagggaaaaaaaggaaaaaattgtacttttttagtatttacctgttacaaaggacattgtgtttcctgtcatgtaaaaccagctattttagttactattgtactctagaaaagagctgtagatttatgttaaactcgtacttacgaacaattgtaattagttctaaaaggcatgaactcagctcctaatcgtcactgtatagtcctgaatttgtagaactagagttaattccctcttggaactttctttgttcttcagtagttacttttttccttacctaaaagggttgtctgtcaaacaattcttgaataaactttctgttatcaattttaaaaaaaaaa

SEQ ID NO: 14 - DNA, 2042 bp, Homo sapiens
ggaaaacgagtcaggggtcggaataaattttagtatattttgtgggcaattcccagaaattaatggctatgagttcttttttgatcaactcaaactatgtcgaccccaagttccctccatgcgaggaatattcacagagcgattacctacccagcgaccactcgcccgggtactacgccggcggccagaggcgagagagcagcttccagccggaggcgggcttcgggcggcgcgcggcgtgcaccgtgcagcgctacgcggcctgccgggaccctgggcccccgccgcctccgccaccacccccgccgcccccgccaccgcccggtctgtcccctcgggctcctgcgccgccacccgccggggccctcctcccggagcccggccagcgctgcgaggcggtcagcagcagccccccgccgcctccctgcgcccagaaccccctgcaccccagcccgtcccactccgcgtgcaaagagcccgtcgtctacccctggatgcgcaaagttcacgtgagcacggtaaaccccaattacgccggcggggagcccaagcgctctcggaccgcctacacgcgccagcaggtcttggagctggagaaggaatttcactacaaccgctacctgacacggcgccggagggtggagatcgcccacgcgctctgcctctccgagcgccagatcaagatctggttccagaaccggcgcatgaagtggaaaaaagaccacaagttgcccaacaccaagatccgctcgggtggtgcggcaggctcagccggagggccccctggccggcccaatggaggcccccgcgcgctctagtgcccccgcacgcgggagccacgaacctcggggtgggggtgggcagtgagtgcaggggatggggtggggggacaggagggggccctggggcctgggccccggaaaaatctatctgccctcccccacactttatatacgaataaacgcagaagagggggaggggaagctttatttatagaaatgacaatagagggccacggggaggcccccccagaagcaagattcaaatctcttgctttctttcttaaaaaaaagaaaaagaaaaagcaagaagaaggaagaaagaaaaagacagaaagagaaataggaggaggctgcagctcctcgttttcagctttggcgaagatggatccacgtttcatctttaatcacgccaggtccaggcccatctgtcttgtttcctctgccgaggagaagacgggcctcggtggcgaccattacctcgacacccgctaacaaatgaggcccggctcggccgcctccgcctctgctactgccgctgctggaagacagcctggatttcctttctttgtcccccactcccgatacccagcgaaagcaccctctgactgccagatagtgcagtgttttggtcacggtaacacacacacactctccctcatctttcgtgcccattcactgagggccagaatgactgctcacccacttccaccgtggggttgggggtgggcaacagaggaggggagcaagtagggaagggggtggccttgacaactcaggagtgagcaggaaaattgagtccaaggaaaaagagagactcagagacccgggagggccttcctctgaaaggccaagccaagccatgcttggcagggtgaggggccagttgagttctgggagctgggcactactctgccagtccagagttgtacagcagaagcctctctcctagactgaaaatgaatgtgaaactaggaaataaaatgtgcccctcccagtctgggaggaggatgttgcagagccctctcccatagtttattatgttgcatcgtttattattattattgataatattattattactatttttttgtgtcatgtgagtcctctctccttttctctttctgacattccaaaaccaggccccttcctacctctggggctgcttgagtctagaacccttcgtatgtgtgaatatctgtgtgctgtacagagtgacaatagaaataaatgtttggtttcttgtgaccagcaaaaaaaaaa

SEQ ID NO: 15 - DNA, 1830 bp, Homo sapiens
gtgaagcacagggttataacgaccacgatccacaaatcaagccctccaaaatcacccaaatgagctcgtactttgtaaactccttctcggggcgttatccaaatggcccggactatcagttgctaaattatggcagtggcagctctctgagcggctcttacagggatcccgctgccatgcacaccggctcttacggctacaattacaatgggatggacctcagcgtcaaccgctcctcggcctcctccagccactttggggcggtgggcgagagctcgcgcgccttccccgcgcccgcccaggagccccgcttcaggcaagcggcttcgagctgctccctgtcctcgcccgagtccctgccctgcaccaacggcgacagccacggcgccaagccctctgcttcgtccccctccgaccaggcgacctcagccagctccagcgccaatttcaccgaaatagacgaggccagcgcgtcctcggagcctgaggaagcggcaagccagctaagcagccccagcctagctcgggcgcagccagagcccatggccacctccacagccgcgcccgaggggcagactccgcaaatattcccctggatgaggaagcttcacatcagccatgatatgaccgggccggacgggaaaagggcccggaccgcgtatacccgctaccagaccctggagctggaaaaggagttccacttcaaccgctacctgacccggcgacggcgcatcgagatcgcccacgcactctgcctgtccgagcgccagatcaagatctggttccagaaccggcgcatgaagtggaagaaggacaacaaattgaaaagtatgagcctggctacagctggcagcgccttccagccctgagcccgcccagaggagcccagcggcccaagagcccgtgccacccccagccctggcccctccaatcctccccgctctgccgccgcccgctggggaccggttcccacaagcctgcctcgccttgtgttacgatatttcgtttggtcttaggtcttcctgtggctccctctctcctggactggttatcttgttattattgttaataataattattattattattttccttccatgctcccaactcccttctgcttgtcccaaatccgccagtgtttctgaatgtttgtgtctgtggttgcagtctttcccccaggaaaaaaaaaaaaagaaattcgcatgtttaatgtgaactctcccctccccatctgtgttctaacttatttataaaaagatgatcgctgtattttgagtttcagctggaaacttctgtaaggggcagcagttgaggtggggtagtgccgcagtggggtcaagctgagctggcttcggagatggagtcccttttcattctcctcctcctccctcctcactccctaggcccaagtctcctaggggcttggtcctagggtgggaaggggctagggaggaccaaagggatggtattgagaagagagaaagaagatagtgagatttaagttcctgctgcctgggtaggccccacaaggcctggtctgggagtatacggaaacaaaaatgatcctcagtgcaaaatgtcttgtgtatttctctgtgaatccatgggtctggctagagggcccaaagcttgtaaatatggggatagtctgggtcagacccatctctcccttacccatcttgcttccaagaccatttgtagtgagcgagtggatgctgtgctacgtgtgaaatctgtctttgcggggcctgtctcagtgattcgcttttggtatttgtttgtagctttcctggaagtcaaataaatgtttcccccactccaaaaaaaaaa

SEQ ID NO: 16 - DNA, 3547 bp, Homo sapiens
gagagacgagggcagcggaggaggcgaggagcgccgggtaccgggccgggggagccgcgggctctcggggaagagacggatgatgaacaagctttacatcgggaacctgagccccgccgtcaccgccgacgacctccggcagctctttggggacaggaagctgcccctggcgggacaggtcctgctgaagtccggctacgccttcgtggactaccccgaccagaactgggccatccgcgccatcgagaccctctcgggtaaagtggaattgcatgggaaaatcatggaagttgattactcagtctctaaaaagctaaggagcaggaaaattcagattcgaaacatccctcctcacctgcagtgggaggtgttggatggacttttggctcaatatgggacagtggagaatgtggaacaagtcaacacagacacagaaaccgccgttgtcaacgtcacatatgcaacaagagaagaagcaaaaatagccatggagaagctaagcgggcatcagtttgagaactactccttcaagatttcctacatcccggatgaagaggtgagctccccttcgccccctcagcgagcccagcgtggggaccactcttcccgggagcaaggccacgcccctgggggcacttctcaggccagacagattgatttcccgctgcggatcctggtccccacccagtttgttggtgccatcatcggaaaggagggcttgaccataaagaacatcactaagcagacccagtcccgggtagatatccatagaaaagagaactctggagctgcagagaagcctgtcaccatccatgccaccccagaggggacttctgaagcatgccgcatgattcttgaaatcatgcagaaagaggcagatgagaccaaactagccgaagagattcctctgaaaatcttggcacacaatggcttggttggaagactgattggaaaagaaggcagaaatttgaagaaaattgaacatgaaacagggaccaagataacaatctcatctttgcaggatttgagcatatacaacccggaaagaaccatcactgtgaagggcacagttgaggcctgtgccagtgctgagatagagattatgaagaagctgcgtgaggcctttgaaaatgatatgctggctgttaacacccactccggatacttctccagcctgtacccccatcaccagtttggcccgttcccgcatcatcactcttatccagagcaggagattgtgaatctcttcatcccaacccaggctgtgggcgccatcatcgggaagaagggggcacacatcaaacagctggcgagattcgccggagcctctatcaagattgcccctgcggaaggcccagacgtcagcgaaaggatggtcatcatcaccgggccaccggaagcccagttcaaggcccagggacggatctttgggaaactgaaagaggaaaacttctttaaccccaaagaagaagtgaagctggaagcgcatatcagagtgccctcttccacagctggccgggtgattggcaaaggtggcaagaccgtgaacgaactgcagaacttaaccagtgcagaagtcatcgtgcctcgtgaccaaacgccagatgaaaatgaggaagtgatcgtcagaattatcgggcacttctttgctagccagactgcacagcgcaagatcagggaaattgtacaacaggtgaagcagcaggagcagaaataccctcagggagtcgcctcacagcgcagcaagtgaggctcccacaggcaccagcaaaacaacggatgaatgtagcccttccaacacctgacagaatgagaccaaacgcagccagccagatcgggagcaaaccaaagaccatctgaggaatgagaagtctgcggaggcggccagggactctgccgaggccctgagaaccccaggggccgaggaggggcggggaaggtcagccaggtttgccagaaccaccgagccccgcctcccgccccccagggcttctgcaggcttcagccatccacttcaccatccactcggatctctcctgaactcccacgacgctatcccttttagttgaactaacataggtgaacgtgttcaaagccaagcaaaatgcacaccctttttctgtggcaaatcgtctctgtacatgtgtgtacatattagaaagggaagatgttaagatatgtggcctgtgggttacacagggtgcctgcagcggtaatatattttagaaataatatatcaaataactcaactaactccaatttttaatcaattattaatttttttttctttttaaagagaaagcaggcttttctagactttaaagaataaagtctttgggaggtctcacggtgtagagaggagctttgaggccacccgcacaaaattcacccagagggaaatctcgtcggaaggacactcacggcagttctggatcacctgtgtatgtcaacagaagggataccgtctccttgaagaggaaactctgtcactcctcatgcctgtctagctcatacacccatttctctttgcttcacaggttttaaactggttttttgcatactgctatataattctctgtctctctctgtttatctctcccctccctcccctccccttcttctccatctccattcttttgaatttcctcatccctccatctcaatcccgtatctacgcacccccccccccccaggcaaagcagtgctctgagtatcacatcacacaaaaggaacaaaagcgaaacacacaaaccagcctcaacttacacttggttactcaaaagaacaagagtcaatggtacttgtcctagcgttttggaagaggaaaacaggaacccaccaaaccaaccaatcaaccaaacaaagaaaaaattccacaatgaaagaatgtattttgtctttttgcattttggtgtataagccatcaatattcagcaaaatgattcctttctttaaaaaaaaaaaatgtggaggaaagtagaaatttaccaaggttgttggcccagggcgttaaattcacagatttttttaacgagaaaaacacacagaagaagctacctcaggtgtttttacctcagcaccttgctcttgtgtttcccttagagattttgtaaagctgatagttggagcatttttttatttttttaataaaaatgagttggaaaaaaaataagatatcaactgccagcctggagaaggtgacagtccaagtgtgcaacagctgttctgaattgtcttccgctagccaagaacctatatggccttcttttggacaaaccttgaaaatgtttatttaaaaaaaaaaaagatgacaaagaaaaacagagagagagaatattggagatgtcctgaattttaatagggtacgcgccattagggctttttgcgctaaaggatgaacatgtactggtttatgtggacaagccattataccaccagactgcaatgccagtttcctctactgcaaacagtgttctgtgacaaaaaaaaaaaaaaaaaaaagaaaaaaaaagaaaaaacagaaatatatccagctaacaagaaaaaa

SEQ ID NO: 17 - DNA, 9512 bp, Homo sapiens
aaccctgctcctcgctgaagatggaggaagtaaaaacaggattacccttagctacagatccactgccttagtttccaccaccaactgcagtgcacaaacacacgttaggcacaggaaagaaagaaagacagaggacacattaacagtaaacacaaacaaaagggtgatgggattattttactgcatgcactgctgagcccgacattgtcacctcctctttgaggggttagaagaagctgagatctcccgacagagctggaaatggtgatgaatcttttttaatcaaaggacaatttcttttcattgcactttgactatggaaacagaggctattgatggctatataacgtgtgacaatgagctttcacccgaaagggagcactccaatatggcaattgacctcacctcaagcacacccaatggacagcatgcctcaccaagtcacatgacaagcacaaattcagtaaagctagaaatgcagagtgatgaagagtgtgacaggaaacccctgagccgtgaagatgagatcaggggccatgatgagggtagcagcctagaagaacccctaattgagagcagcgaggtggctgacaacaggaaagtccaggagcttcaaggcgagggaggaatccggcttccgaatggtgaacgccccttccactgtaaccagtgtggagcttcttttactcagaagggcaaccttctgagacacataaagttacactctggagagaagccgttcaaatgtcctttctgtagctacgcctgtagaagaagggacgccctcacaggacacctcaggacccattctgtgggtaaacctcacaagtgcaactactgtggacgaagctacaagcagcgcagttcactggaggagcacaaggaacgctgccacaactatctccagaatgtcagcatggaggctgctgggcaggtcatgagtcaccatgtacctcctatggaagattgtaaggaacaagagcctattatggacaacaatatttctctggtgccttttgagagacctgctgtcatagagaagctcacggggaatatgggaaaacgtaaaagctccactccacaaaagtttgtgggggaaaagctcatgcgattcagctacccagatattcactttgatatgaacttaacatatgagaaggaggctgagctgatgcagtctcatatgatggaccaagccatcaacaatgcaatcacctaccttggagctgaggcccttcaccctctgatgcagcacccgccaagcacaatcgctgaagtggccccagttataagctcagcttattctcaggtctatcatccaaataggatagaaagacccattagcagggaaactgctgatagtcatgaaaacaacatggatggccccatctctctcatcagaccaaagagtcgaccccaggaaagagaggcctctcccagcaatagctgcctggattccactgactcagaaagcagccatgatgaccaccagtcctaccaaggacaccctgccttaaatcccaagaggaaacaaagcccagcttacatgaaggaggatgtcaaagctttggatactaccaaggctcctaagggctctctgaaggacatctacaaggtcttcaatggagaaggagaacagattagggccttcaagtgtgagcactgccgagtccttttcctagaccatgtcatgtacaccattcacatgggttgccatggctaccgggacccactggaatgcaacatctgtggctacagaagccaggaccgttatgagttttcatcacacattgttcgaggggagcacacattccactaggccttttcattccaaaggggacccctatgaagtaaagaactgcacatgaagaaatactgcacttacaatcccacctttcctcaaatgttgacataccttttattttttttaatattattactgttgataattcttattttgtggaggcagtgtcatttgctctgcctaattacgataaggaagaaacagaagagagaaggggcgggaatattgtttctttatcacctggcttgtttattttgtgggaatttaagagcagtccatttctaccaaggcatatcatgctttgaaaaatcacttgattcataaagattcacctaagagattctgatttgccactgatattcagaattatgatggaagacaggaaagttcagagttttctgggtaggactttggtggtttaaaaatggtataagtaactttattcttgaaagaagaatgtgtttcaaactgtaaaccaattttttgttcttcagagatcatggaacacaaacacattgttattttcagtgataactcctaagaggagctgagttgttgtgggttctatgtttacttcccctatggaatttataattcagtatgttttacactgtaccatatagcaaaacttttaaactacaggtagttaagggccacctacaatacatctgaggtcctgtgatcttatttttctaaacgtaagcactgtttttccatagttttgatgactggcattttatagacaccctggcagccttacttttaacacctttaaggaatagtatttttatgtagttttcagaataacatatggtctaagagtggataaaaggcagtcaataatttctgggagggacttctactttcataaatttgtttgagaggttttcttttaaagttgtaatgtgatggcagcatagtatatgtatttgtttctaaaagtatgcttacgattgtcactttatcagcatttaatcagtgttaaccagtcagcagaaaaatataattatgctaacagtagggggagaaaacccacttagaaatcccttttctggtatttctcttttcactagtttttttcaagatgtgacctcccggtgttctgtccatagttcattcatcctttactcttcgagtagaaggtcttaaaagtcttcctgtcggctgtttctttcaaaatctcctcagagcaattgctaatttggcctgaatctggtaacttgaaccctgtaaggttacagaactagggctatttattttagcatttcttcagtagtatttactactcttgttgcaaagaaaagggaatgggacttctttgtaacctgtaccttggacaacagataaaagaaacaaaaaaataagaaagtttacttttacccttcttggagtctagaatgtgacagaacccccaaaggaaagtcctgcacatttttctgtttccaaaacatttaattgtgtaagtccttgtcagaaatgaatctcaatcccttagtatagaattccccttacatggtataggttgccatatttcatgtgcagattttaatttcatttatgtgggcgctctgttttttctttgcagtccagccacattagaggggaggaaccgagtgatattgattcaagtcattttagggggacatacttggaaggcagaacttgctgcttctgtttggggaggacagacctgactgtgactggattatctgataaccatttgtgaatactgaaattctgttaggcagtaactgataactgctctaaaggatcattaaataggatgctgaaattatgtatcttaatacagtgtggtatgagaattaccaagtcaagagaattgtggacataagcaagtttggccccaatactgctcttaactcattttccagcttactatttgctatttaaatggtaggcaccagctaagcacttctaagcactaacacagctagaactaggcaaaaatggttagaactcagctctcttctactagtccctgtcataattatttttgggaaaatgtccaaactgccccctttaaatctaagggaatgcaccaaaacagagatatatagaatgtcaaccatttcatttttttttttctgcatgccttggtacatagtgaacatacaacctatttaaagataaagcatgtttttgagactcgctcacccccccccacccaaccactcccaaataataattgggatgccattttttttccttttggatgaggtaaataattttaaggttcacaattttgtcttttactgcaatttaaggaaacatttggatgtcagtcaatatgttcataattttggctgtgtgcgaatttctgctggcattatctatgaattttcttcctacttatttttttttcagtatatgaacaatcatgtatctacctgccccaggatgaaactaaatttaggtggaccctaaaccttatgaagacagtgctgaggcactttccttttctgatttcatctttttgggaatctgttttattgaaggtagttagtagttgagagtgcatttgctacaagcatatacttgtatcttcctagcttcatgaggaacagaaagaggtggatatggctcagggtgtggcagggacaattgaggacaaagtcaattcaaatttgtgggtcagaaagaatttttgtggacgtagtgtttttggagaaactctggatggttatatgtgcatgccttttcttcaaaaggaaatacgcaaggttgtagcatctaaaaataaacataagagtcagacaccaaataaatcaagttttacataacagttgtatgcccagtttgtttaggtgagatttcacattacagaaagtatttgaggagcatgaaaatgggttatcttctgtattttccagtttggcaaaagttcagaatttcatcacattgctttgccctaattttgcccagaattttatcttagcctctctctgacagtgatgaatcatgctcaaaagccattctaattggacctttttaagacagggaaagggatcagtaggcggattggaagaaatttcaagtcattgaaatattccattgagatttcctaaagggacaaaattgggaaaataagaaactacgacttagatttggctacgtagtagaaagtatctcccctacatacatacaggcaattgtatgtatgaatcatagggtatatgtgtgtgtatactacacacacattcttttaaagagaattcatggaaaaaaaagcagttggagtgatcagatgtattgcaaaaacatacagagaatttaaatgacagttaataccaagaaattagttgggtttactttatcaggtcgtaataggaatcactaaagaagttactagtgtgtctttaggaccagtggcaactcttaaactaaaactttgggtccttattatctacttacagaacaaagtgaaacaaacaatgattaagctgattggatatacattcaaagatatttaatgtaaagttttttggaatacgaagaaaattcagaaaataaatattatcaacagttacttattggcaaatagagaaagacaagaatagtttagtgagcccggtattttgtttttatagtttttatctcagttgtacaactcacaaaaccatgaagtctttggtattttataaatgtttaacaaaatttacatcagattaaggcatttagatgaaaattattatgttctcactatcttccaaattttatttcatcctatctccaaaatgatttcttagggtacaaaaagagcagacggggctgtaaaaatacaagcaaaaaactgtgtgcccctagtttcaggcagaacttaaactgtcagaggtactagctacatgatttgttttttaactttggattgttcacgtccaaaaatggataaattacatttgtgtttatcatcagttgcattttatgtattattttaataaatactatctgaatgaagactattctaaaccagaaaattccccaaatccaaaagaaaaaaaaagtgggaagaggtgaaattgaagtttgtgtatatgaaagttatcttagacatatttttaattctccagtttctgcaaaataattaaaatatacagtaactggtctcctaaatcctgaatttaatgtattaaatacttatgttctttatattggtgcctttttaaaatgcattgagagtgttggttagctgttgcagctgtacaacacttttaatatgcatttttaaaaatcacttaaaattgagtactatataattcatctctgcatttttagtgcaaatctttagagcaatttctaatagagaaattttcagctcagctgttaaaaggaaaaggaaactttgaaactagactttactacctttttagtttcatagtatttctgaatatgattacaagattatgcaggtaaaatatagagtgaaactttacctgtgaattgaattataatttgtgtttttgttttgtttttaaggaagaataagttctgtatcaaacaagaatttattagataattttttggtcaataaaatacagtattcatttggattttcatctccagactagtattgttctagtcttggaatctgtattttctaatctgttagaaaatagagattgaaaattgatggaataatgtgaaaaagcaggtaattaattctccttgaacaaagcaaaactgaacagtcatatcacattgctattctccaaagcataatctcaaatggtttcatatcatggttgtgtattacttgcaatgggtgtgttaggatatgacagctttttaaaaaaatgagctgctggttatacaaagcaaatggcatatgaccaagaagctgtgatatgctagtgtttctttttatcatagtgtattactaggccaaataatgacaccttgaatatttttacatttattgcagaaaccttaaactttggaatttccataaggtttttatgtaatattctatttctagctttttagttttatcttgctgtactgtaagtttgaggatatttttcacctgcactcttaggaataagttcataattctgtttatggggctttcctcccataacactgcatttgtatattttctgtataaaatatgtgttgtgtattaacctttatcccatacagagagtggtacatgaatgactagttttctaagatgtcctttttattgtgaataaaatataaaagttaaaggccctctgctaagtcacataaagtacagcatataagttcatataggtacaaataaatgagtttgcagtgaattgggccttcaaattacctcaagtgacagatagtaagaaaagcttcttgagcaggtggaggtcactgaatcccctactatgcacttaccaagattttacttactttaatttactggaaattgattttttaaaaaatgactacactgtaacaagggaagggatctgggtttttttgttgttttattcttgttttttttaagtagttcaaattctgaaactgtgatttaaaaattttttacagtcaagcattctgattttgaacataactcccttccctttctgtgtaacaaaggtctctctgttatctcttaaattttgttacatctccctcagcctctttctttgtccgtctcccttctgtcattgtctatggatgtttacctctctgttctcctaaaagtttgaagattaggtcaactcttatttctagttcattggtaatttaatcttaatttttttttcgtgatttttgttggttgtataatctgctgacgtatttttatactcaagtgtagttttctattaaaaagaaaagtggttggattaaaaatagtaagctatgtaaccctcatgttactttcactttcaaatattgggtacctaaaacattacttcagagattatgtaatcctattatagtatgtttgctttcctttattgttggattttacattctgatttggctttcctccaaaaaatgtatatcatgaaagactagacagttatttgcaagtgtttagaaaggtgttaaaaatgtaaagcaaagagtcttaactttctcctaattgggagaaaaatgctttaacattactataataatattccaggtttggagggggtctccaggccccatatttgctgttaatagttggaccttttagaccatgtgttatttgcaatcccagaatgattgcttctgctattagttaaaaagatactattcttttctttctgtacaagtgcaatactccccttgaagtcttaaaaactatggtgatttttttttcttttctgacctattcttcctttagctaatgacaaaaagaaactcataaaagtcatagtatgttaaaggacacaacaagcaaagagaaaaacactccacaatcaaaagattacagaatgtggaaaccactagtctgatctcatggtatctttatttaagctaaatttccatggaaattagtaatcttttgcttgaaaaatgtgtcctaaagttgaactttttacagattgaatcttcttagaccctcgcccaatgctctaaattaagaacctaatacttaatatttttattttacttctccccttttagaaataaacttttaaataaaagcaaagcacttagctgagttttaaacacttacatatcacctattggagaaattttttttaaaaatatttggagcagtcctgttttcatacaaatttaagtaagaggtatttttcttatacatatttatatgtagtgtgctaattttctttttttatacctgtgtccctgtagtaaaactgctgtaatataaatacatgttttgttaaaagataacatttctttggcatttcttttaaaggcagttactgcatttctgcatttgtacagtatgtgtcttggccattttagatattctttctttaacaataccaaaggtaattagactattttaaagactaattgcttgacagtttctagggtattttgtgttttagaagcaaaaaaagaaaaaaaaataggtcaaaccagtaaacctcattttttttcaaactaataatttggggaaataaaaactattgtttaaaaaagaaatatatatatatatatataaatatatatgtaaagttaaaattccataccttgtatgtcaggtttgctaagtgtaatgtagtttttttaaggctcaaataccatacctcagaaaatgaggtttactatggaaatactgaaacagtctttgcagctgtgtgacaagtcactctactacatactgatttggagacctccgctaaatagttttatcactgcagactaaaatgtgggacttgtatcttctttgtttttaatgcacacacatacatgttctgtgcatgtatgtggttactgtgtatatgtgtatgagtgttgtatatgcatgtgtgagtgtgtgtctgtatgtgtgtacaactaaagaagctgcagaaactttgtaatactttgtgaaaaggattatattataaaggtttgtactgtctgagtgcacagctactggaataaatttagggaatctcaggaacaagcatataatttgtccaagatttatttcttctcagaagtgtaagtgcagtttttaattctgtatattatttaatattttaccaataaaataaacttctgacataaaaagtttgctataaaaaaaaaaaaaaaaaaaaaa

SEQ ID NO: 18 - DNA, 10923 bp, Homo sapiens
attcatttcctgagaactgcagagagccgctgagaggctctgcgtgtgcgtgtgcgcgggtgacgccgtgtgtgtgcgagtgtgtgtgtgtgtgtgcgcgcgcgcgtgtgagagagagaaagggagagagagagggggactctgtgtgagggaaagaaaacaatttctcctgctctgcagcttctgttcaggatcaatgtgactctaagaacaaatggatgaatgaatatccatatgaagagaaaaacaataaagaatatcaacacctttgagaacagaatgttaatgcttgatgggatgccggcagtcagagtcaaaacagagcttttggaatctgaacaagggtctccaaacgtccacaactatcccgatatggaagccgttcccctgttgctaaataatgtgaaaggggagcccccggaggactcgttatctgtagatcacttccaaacacaaactgagccagtggacttgtcaataaacaaagccaggacgtcccctactgccgtttcatcctccccagtttccatgacagcatctgcctcctcaccttcttcaacttcaacctcttcatcgtcttctagtcgtctagcctcatccccaactgttatcacatcagtatcttcagcgtcatcttcgtcaacagtattaactccagggccccttgtggcctctgcatctggtgttggaggccagcagtttttgcacattatccatcccgtaccgccttcaagtcccatgaatttacagtctaacaaactgagtcatgttcaccgcatccccgtggtggtacagtcggtgcctgttgtctacacagctgtaaggtcacctggaaatgtgaacaacactattgtcgtgccgcttttggaggatgggagaggccatggcaaagcacaaatggacccccgaggcctatctcccagacaaagtaaaagtgacagtgatgatgatgacctgccaaatgtgaccttagatagcgttaatgaaactggatctacggccctttccatagccagagcagtacaagaggtacatccgtccccagtatcaagggtccggggcaatcgaatgaataatcaaaagtttccttgttcaatttcaccatttagtattgagagcacaagacgccagagacggtctgaatccccagactccagaaaacggcgtatccacagatgtgattttgagggatgcaacaaagtgtacacaaaaagttctcacctgaaggctcaccggaggacacatacaggagagaaaccttacaagtgtacctgggaaggctgcacctggaagttcgctcgttcagatgaactgacgaggcattaccgcaaacatacgggagtgaagccattcaagtgcgcggactgtgatcgcagcttttcccggtcagatcatttggccctgcaccgccggaggcatatgttggtgtgaggaatgctacctgtccagctgagcgtaagagctggatctcttagcggcacccaattcagcagggctgaatccccttcacagtgttaacacaaaagggcatcaccatcccacgatgtctgaaaccagagcaggaaaaagaaggcacacttcttttggtctgaaggtaacccccatcatgactagacgagaaccgtctttacccgggcctgggagctcagtgacataaatgctgaagagacaggcattgttttccgtgctgtgtcctctgccatttaaagatgtttgtagcttgtacattttctgagctgtcagacattttgttattgataccttaaaggtcacctaccacaaattgatggctagagcaagaccttttaaatttggatgaatctcccatcatcccatcccttaacccctttttacagaaattttagttacaatctgagtaagctagaacacacatccaatctgttaccagcaagcagcatgaaagtagaaacgaagaagcagatggagaggtcccattacctgcaaagaaagggcactcaggcagccctttgcaatcgcgatggcagccactagatatcactctcaacttgtcattctgccatgccctgaagaaaaccaacattgaatctttcatttgtttgtcgttgattgtttttgttttgtttcgattctgttttgttcatctgttcgagcagaggggcagttgaagtctcgtcctggtctctgccctggcatggactggcacagaggtgttctgtagttgaataggaagagcctgtctaaaaaactactgccccacttcaaattgcagtgttctgtcacctaggcatcatctcttcctgcccctagtatttgattacaaggaaccaggggaaaaaaactttcttagacacactggcaccaaggtaagaggtggggctgcccaggcaaagtcagtgaacatgaaaactcagacaaagcagagatggaaataatgcgcctcttgaggagaaaagcaataatgaataaaaggactttcctacaataacttcactgaggactcacgttaccaattttcatacttactaaagggattgtaaaaaacaccccagcattttaggtgtcttggttccatttacagcactgaggtaatctttctgctgtttgttgtcctgcttggttgagtaccacaattaaagattatgctccccttttcttgtttgaaaacagttatttggtgactagaaaggcaagggaggcatagccgggaattaactcttcggttaatgggtcagttctccttggaactgaatcagtggaaagagaatgcttcccatagaaagcccgacatggtgctagtttcctctcttggagagccaaggataagtgactctccaataggttcttctataccgatttcattttctaaactgtatcaggacccaggtgtcctgcattggcatctgcagggatattgaacccactcatcctgtgcagaggatgagaggaaggtgttagttccatcagccccataaaattatgaacctttttgcaactaggaacatatacagtaaacaagcaatacatcaccaacgagcattctttcagaaaaattaccatatttttgtccccaccacaagctgggtttttttccagctttctgtggctgaggggctggtgagattttttgttgttattgttgttgttgctagaattttttatagcttagatataaaaggtctccaacaaagacctttgcaatatattttaattacaaacacttgattttgggaattgcacatattaacctcacaatattactagctcatttttaaatgtctggacattctgaaataatttccggttacagaattaccctttttaggatcatcttctaattcagataatcttattctgatgaagcgagaactatgaacgttgaaacaaggacaatttgtgttggatggtattcaaaaggaattttttaaaacattaagcaacagtgtaatgattttcaggtcaaattatggcttcacaaatatgtgctctatattgtctgccaggtctcaaaaattcatcaagcaatttccctccaacaagggctgcagttctagtgataaatagcatctttagctgcacagtctactactcatcaaccagtgggttttttttagcacctggaaagtttttttgtactatatgcaactcctacttcacattgcctgtgaccattcagaatagaaaaaggtctgttgatgcatgccagtgctggttactagtcagtgacagagaacacagagagggacaaacacagccatgaaccccctggcactagtaccagcacctagttggcaagtagtgggtgcatctctcaagtgaagtgtagatttgtatttagtcctgcttgctctcataaaaagagccagttcttttctctgctgaaagatgtcttgagagtattacaggtagacttggtttttagaattcataaaactgacaggcaggggaatcttcctgagtgacagcttggtttaaatttagcacagaactagatgagttgcacttttctaataacataggatggatttggggaagggaacattttccctcatcctctgaaagtggaaaccagactgagatggtaagttaactgataaaacctgtcagcattccatcattttccccttttgacgtatcagtacttctaagaagggatctaagcacaattaagtgaattgagtccctggtctagcctcaggaactgaaacttctttggggcttgtcttgagacgtggcaaattctccttggtgagttttctaaaagtatttccccatggaaaggagaaagtggtttgggacagaaagaaatggaatgtcttcagtcatggaagagcctccatttcatctcatgtccatttctagcctctttcaaaaagcacagtgcatcatgagcaacagaaaagcagtagaatcctaaaggacataacagttgttccgtaactagaagcattctttgattttaaatttgaagctgttcagtttgcagctggggatatgtgcatggttgggaacttatacacatagagccattaactacttaagcagctgcagtagacctgattttctgtttgcagtatagagattgatttcaagggagttacattatttgaaaagttatgactttcagcctttgaggttttaagaatcccttttcatggtctgatcttaagttgtttgaaaaaagattcacgcttctgcatccacctctgccctcattctgtcaattgtgtgtggactctgagaagcctgcttgataacttgtcttaagttctattaaggcagttagactgagatttctaatgacttcaccataaagattatacttctgcctggcgtcgtttctataaaacactataggacatttgttttcaaaagcagttgagtaacagcaacataaaccacattttttaaaaaaatgagcacttttcattcacttgaaagcttaagtgggaaaaaggaagatgattgtcaggggaaaaacttacaaaatgctacagtgtttacaaatgccagttccaggtgattagaaaggccgttttcattatggttcagatttggaatagcttatgcaaagattgtgttcatttttatacaattctttttaactttaaattgccatctgtgttttaaacataattctgcactttggaatcagttcattagaatgataacatttatatggataaagatgcagtattccttattctgtacttgatttattatacttaccaaagctgcaactgaataaatctggtgaggtgaggccttcaaaatgtattaatacgcctttgttatgttgttatgactgcttagaaatgtagccatgatgttgttctaaagatgtcttaacatttatagtgaggattgacagaaattaagcagtgttaagaataccagcatccatgttacagtctgcgtataagggactgaatgtgaggtaactcttatgaatcataataggcccagaaagccttaatattcatagttcataatccagtctcagattttgctcttcaatgttgcctgtaaacttagcaggtaaatgtattctagtataaaacatcattgaccaaatctcttcctttagtttttctgaggtagtgcatcccattaaaaaagtctcttcaattagatttggctttagtttagaaaggggtttggtaagtcatgtcttccatatgacatttctactcatccccacactgccttccacttaacctgtacagacagccccaacacatgcacatgtgtgtacacaactgtatatgcagcagccagtgggctaacaaatatcagcaaagaacttttttaaaaagaatttgtaataatccgtgctgtctagaaatgtgagttacttaccttattaggaaaccggcttataatactatatatgactctgcctgcattttatagttaagaaatgtacataaaaattgctatattttcacagtttcattataatgtctgtttagtaacccatgtccactgatactttaaatatgctaattactgagcaattgcaatagaaatccagattttcataagaaaatgaaatagaaggatactgcatctttaaaaaatagagtgaagatattttactgtattcgtaacatatgtgatagtggaaaaagtattttcaaactcacaaattttacagaggttgtaaatagtttgatgaagtatgttgggaaagagagcttctagttttcatcacaataaaaagaaaaaacttgttcagatatgccttgtatatcttacacacaatgagtgaaattgccatgggaactctgtccaactttgtcagagcatgctcttcgtgttctgttcaaaatagagtggccacttgtcatggtttagccaggatggtttaaaatataatgaagtcattttaagttgcagacaactgattccaaactagtaatgtcttttcctgaaaagggcaaaacagctgcacaggattttgcttgtactaaagaggagcatcttacaattgttctcttttctcagttatctccctcctcaacagagtgtgaacaaccatacaggtgtttattcttgttttattccagggagactgcttttaggtataactacatgttgacaagattaagtgctgtggatatggccacgtgttaaaggaatctatactcgcaataggaataagctaatgcccattttcagaaataaattaaatgctgaaagtgcctgtcagtaaaccttatgaccaatgagatattcctatacttttacacagcaataattcttaataagactgggttttttcctgcacatgtgaacacacacacacatacacacagagagagtaaatacatacacatagtaaatcaaatctgttcacatgcccacaaaaaacgcttttatagctttcaaatatacctaatcttgtttacattaacatttcttaatttgatctttctaacttgcagaacgttaagaacacaaatctgaacattaagtttatcttcagtgaacactattgcatatggtaattataatttccagataaaaagttgactgtcatttgccccaaacttatgaaacagtatgtatgattcaaaaagttttgctcagttaaaaacaccaatttatttggcgcaaaagactgaaggatatttgtttgcttgttcattttatccactcatatcaaacgcacaaattcatctttacatttcttcattcccctttcccctaatatttcctattatcatcttaagaatcattttggaatgaatattgacagaatcatttctccccaaaaagtctgttgaagtaccactgacacttagtaagcctagcctttaaccatctttgcagttagtcctaaaaattaccagatgcacattcagaagctcacaggttttttcctgtaattactgtactgctaaaagtggagtcatggatagaaatatacaatgagactatatgaatagacatgaaccctggaaattctccaaccctgccatctcagagcacaaattcacccaaacaagctactgaggctatcagtaaaattatactcatggagagtcacacatcgggtaagggcaattttaaaagtaggactgtgattaaacattcaggctttagacttccatgatggactcagaccacaggcctcaaaaaaacatccactgttttctcctcccaatgttagaaagaagaaaagggagacggaaatgtaccaggataagggggtaaaataaccaattaagtgtcaaaggaaagctgctgggttccttaatctcactgaagtcaagtcttctgactgggcttcttaccatgtgttgtctgaaaaaactaaagtaccttgaaaggttacacattcagcaaaccatgaagataatagctattctttattaaacactgtgtgccaagcaatagactaggcaatttttagatacgttacctgcaacctgtacaacatttctacactttatggatgggaaacggagacatgggaagtgtggctgagttgttcatggatgtagaaatagtaaacggcagagtaggaaagtgaaaccgcctatctctgacctggaggtctgcctgtatctttcccactccaccacactgcacgtgggtgtcccgaaaccaccttcccagattcctgactctcagtaattttattatggacaacatgcatgagtagtcatcatatttttcaagtgaaatatcgggacatgatataacacatgacttaacaatggtactgaatatttgaaatcaggcctttcccggaaaatcatgcatgaaggatcattataaacaaacaatagcaaccagttgtctccccgaacttgtcacttttctcataaatgtctggcctggagctccaaaatcatccaaatacttagtagcattttagcctgagtacactttctcagttcctcaactctttgtatacctttccaccaatatagacattctagaatctgcttcagatgcatttgaaattttcacccccatggaactagtgattaatatcagagcccactcttgcagttggtaatggggtggcaatcaaacgttcagatgatgataaaggagagataatggataattctttttcagagttctcacttaacagctctgttgtggaatgttttaaatagtcttataaataatttgtttatagtattgttgttagtttaattgaattttatgtaagaagctgtccaacatcagagaaatgaaattcctcccactttctgtgtagaacaaggtctctgacagtattgattcatggaagtactaatggacttagaaaacattaagagaatgtcatttctcatagtgtttctgtttctgaaaatgaatctcctgaattattatctttctccctgttacttggctggggaaagagatagaagctgtataaacaaattctcttccatgctcaaagcaagtgttccatgtgcacaacctgctgcagactggggcccttctcagttaattgggtttcacaagcaataatttctccacaacaaaaaccacaacttgaagtgagttgaaaagagatcaatagtggaaacagtcgcctcagtactttttctttctggatttcatctctagaaatttgaagtgtttgagacagagtccaccctttgtgcaaggcgagaaccaatgaatggactccttgtgtgaattattgcatcttcttccaaagcaggttcatcaagactttcacagagattcatttttgttgagaagtaagggttaataggaggatagaatttggatccaaatctagtgataaaagtgtccaagcaatcaaaaagtaagatattttagggacataccaacatcttccctttctgctaatttcatgctccaaagatatggcaaaaaaaaaaatcataaaaagtgcttttgccctacttgtgttctagttttcccatggcagaattttgtaattacatccagaatatagtgtatattttgttcctcaaactttattacattggatggatattgttgaactggggcactggtgcctatattcaaggctctttcctatcaacgtgtctgtccacgatttgttgtgtttaaagcttcattttgaaaaatcactgtccccctgtggggtagtgactgtattgttttgttcatgtctatgtgggacacattgcatcacatggcaaaccaactctctgtggatgtgagataagtacttataaaaccagcttgaaaacatcgtcttatgtattatgtcatcctgcatcataatgcaattatgtgtatcataacatgctcatttaaaaaaagagaaaccagcaaattcatgtttgtccatagaagaatgtactcagaactttgtgttgtgaaacgatgagaacagaccacctttaagatacccacctgccacttaaaatgacttagttataattagtagtagtctagacgttgttcttggtgtgtgggggtcaattctaacgtcatgttcttttgaataaatctctcagtcatatttgaaaaaaaaatacatgggaataaagaaaaatatcatctttggccaaatcaagcaggcatcttttttcttttccttgacgtttagctcattatacgtggtgattggatcacgagatctgtccgtgtgaaaatacagaaacatcctttagtttacaaaacagttattctaggcttgaagcctctgaacagcaaattgaatagatgggctgcatctgatttgctttatggatgtaattttacaaaacactcttgggtctctgaccccagggagttaagagtgcccagaggaggtcctacacattaaaggataaagccccccagtgatgctggcaagcaaatgtgttgagttcttaaatcttcatttggttttctgttcaggattttaattgcaaatgaatttatttctccagtttatctaaagacctaatttcccaatagtttcctctgcatttatatactctgtagtgtttaggcaaccctgttataagtttattaatattatgtaagtgttgttcttgtatttatgtatagtgtatgtattgtaaatatactcagagcttttttccttttactgtaaaatggtgatttttttgccctatgataatgtaaagggagaccctcctaatgagattctctcagaggatgattattccagtctattctcagagatttaaatgaacaagtgttattgtttttaatggtgtctcagacatattctgttggtgcattgcttttctgtattcaactttcctatgaattgagctgtgaactgaaatagagtttaaacctttaactgtatgcatttgtataattatctgaatgaaggcatgaaggttaaataaagcattttgtatggaacaaaactccctaactgactcagacactttggaaatcatagttattaaccattctaattaaacctttgttatgaaaagttttaaaaaaaaaaaaaaa

SEQ ID NO: 19 - DNA, 2949 bp, Homo sapiens
agtttcccgaccagagagaacgaacgtgtctgcgggcgcgcggggagcagaggcggtggcgggcggcggcggcaccgggagccgccgagtgaccctcccccgcccctctggccccccaccctcccacccgcccgtggcccgcgcccatggccgcgcgcgctccacacaactcaccggagtccgcgccttgcgccgccgaccagttcgcagctccgcgccacggcagccagtctcacctggcggcaccgcccgcccaccgccccggccacagcccctgcgcccacggcagcactcgaggcgaccgcgacagtggtgggggacgctgctgagtggaagagagcgcagcccggccaccggacctacttactcgccttgctgattgtctatttttgcgtttacaacttttctaagaacttttgtatacaaaggaactttttaaaaaagacgcttccaagttatatttaatccaaagaagaaggatctcggccaatttggggttttgggttttggcttcgtttcttctcttcgttgactttggggttcaggtgccccagctgcttcgggctgccgaggaccttctgggcccccacattaatgaggcagccacctggcgagtctgacatggctgtcagcgacgcgctgctcccatctttctccacgttcgcgtctggcccggcgggaagggagaagacactgcgtcaagcaggtgccccgaataaccgctggcgggaggagctctcccacatgaagcgacttcccccagtgcttcccggccgcccctatgacctggcggcggcgaccgtggccacagacctggagagcggcggagccggtgcggcttgcggcggtagcaacctggcgcccctacctcggagagagaccgaggagttcaacgatctcctggacctggactttattctctccaattcgctgacccatcctccggagtcagtggccgccaccgtgtcctcgtcagcgtcagcctcctcttcgtcgtcgccgtcgagcagcggccctgccagcgcgccctccacctgcagcttcacctatccgatccgggccgggaacgacccgggcgtggcgccgggcggcacgggcggaggcctcctctatggcagggagtccgctccccctccgacggctcccttcaacctggcggacatcaacgacgtgagcccctcgggcggcttcgtggccgagctcctgcggccagaattggacccggtgtacattccgccgcagcagccgcagccgccaggtggcgggctgatgggcaagttcgtgctgaaggcgtcgctgagcgcccctggcagcgagtacggcagcccgtcggtcatcagcgtcagcaaaggcagccctgacggcagccacccggtggtggtggcgccctacaacggcgggccgccgcgcacgtgccccaagatcaagcaggaggcggtctcttcgtgcacccacttgggcgctggaccccctctcagcaatggccaccggccggctgcacacgacttccccctggggcggcagctccccagcaggactaccccgaccctgggtcttgaggaagtgctgagcagcagggactgtcaccctgccctgccgcttcctcccggcttccatccccacccggggcccaattacccatccttcctgcccgatcagatgcagccgcaagtcccgccgctccattaccaagagctcatgccacccggttcctgcatgccagaggagcccaagccaaagaggggaagacgatcgtggccccggaaaaggaccgccacccacacttgtgattacgcgggctgcggcaaaacctacacaaagagttcccatctcaaggcacacctgcgaacccacacaggtgagaaaccttaccactgtgactgggacggctgtggatggaaattcgcccgctcagatgaactgaccaggcactaccgtaaacacacggggcaccgcccgttccagtgccaaaaatgcgaccgagcattttccaggtcggaccacctcgccttacacatgaagaggcatttttaaatcccagacagtggatatgacccacactgccagaagagaattcagtattttttacttttcacactgtcttcccgatgagggaaggagcccagccagaaagcactacaatcatggtcaagttcccaactgagtcatcttgtgagtggataatcaggaaaaatgaggaatccaaaagacaaaaatcaaagaacagatggggtctgtgactggatcttctatcattccaattctaaatccgacttgaatattcctggacttacaaaatgccaagggggtgactggaagttgtggatatcagggtataaattatatccgtgagttgggggagggaagaccagaattcccttgaattgtgtattgatgcaatataagcataaaagatcaccttgtattctctttaccttctaaaagccattattatgatgttagaagaagaggaagaaattcaggtacagaaaacatgtttaaatagcctaaatgatggtgcttggtgagtcttggttctaaaggtaccaaacaaggaagccaaagttttcaaactgctgcatactttgacaaggaaaatctatatttgtcttccgatcaacatttatgacctaagtcaggtaatatacctggtttacttctttagcatttttatgcagacagtctgttatgcactgtggtttcagatgtgcaataatttgtacaatggtttattcccaagtatgccttaagcagaacaaatgtgtttttctatatagttccttgccttaataaatatgtaatataaatttaagcaaacgtctattttgtatatttgtaaactacaaagtaaaatgaacattttgtggagtttgtattttgcatactcaaggtgagaattaagttttaaataaacctataatattttatctgaaaaaaaaaaaaaaaaaa

SEQ ID NO: 20 - DNA, 5208 bp, Homo sapiens
cttactcatttgtgtttattcttggacttatcctgacataatggggtttttttaattatagattcacactgcatttattcatcacccctgtcctctcatccataactcaaatttactaccagcaacacaaaatacaaagatgtgtccagtttcactacagctcttcgcgtttacaagtgtcgagcgcttgctttcggaacgcccttgtgattggccgagccaatgccagtgacatcaaccaacttacttttgattggaaggctggttgctgggactgtagcgtttgcaggaagtcacttaactgtttgggagctggaaaaccgaagctgaagttctcttttgccataggaacgagcgcaactgactaggaaagatgtgtcccaaagctccgcaagctggaacgtgagccaggaggcccggaccggccacgggaccgcgaggcactccgaaagtgtgcggctgccccttccctgcctcccagctgttacccttttaaatgtcagtgttcgaggctgtaggggtagcacgaggcagcgaaacggaacagtcggattggccgcacgcctcagttctagacgcacctctccaccgaaggccgttctgactggcagggggagaaagtaaacagagttgaatcaccctccccactggccaattggagggggtttggtttgtgacgtgatgggattctgcgaaattgttactgagcaagagaatgccggaacggtgcggaccggccggagcaggggttcagaagccgtcagtggactcgggaaaaagtgtctcttagacctggcgctcggcgggaccctcgccacccgcgtcggggtgatcgggtgaatgtcctggggctttggctcgacggcgaggcggccgagggcgtgcacctctcttgcagtttcctctcccagcgcctcgggggcgttttcagtcgaataaacttgcgaccgccacgtgtggcatctttccaagggagccggctcagaggggccggcgcgcccgtcgggggatcgcggccggcgcggggcaggggcggcggctagaggcggcggcgcggcggagcccggggccgtggatgctgcgtgcggaggcgctgccggttacgtaaagatgaggggctgaggtcgcctcggcgctcctgcgagtcggaagcgccccgcgcccccgcccccttggccgccgcgccgtgccgcgccgcgccgcgctcgtcgtccgaggccagggcagggcgagccgaacctccgcagccaccgccaagtttgtccgcgccgcctgggctgccgtcgcccgcaccatgtccgcggccgcctacatggacttcgtggctgcccagtgtctggtttccatttcgaaccgcgctgcggtgccggagcatggggtcgctccggacgccgagcggctgcgactacctgagcgcgaggtgaccaaggagcacggtgacccgggggacacctggaaggattactgcacactggtcaccatcgccaagagcttgttggacctgaacaagtaccgacccatccagaccccctccgtgtgcagcgacagtctggaaagtccagatgaggatatgggatccgacagcgacgtgaccaccgaatctgggtcgagtccttcccacagcccggaggagagacaggatcctggcagcgcgcccagcccgctctccctcctccatcctggagtggctgcgaaggggaaacacgcctccgaaaagaggcacaagtgcccctacagtggctgtgggaaagtctatggaaaatcctcccatctcaaagcccattacagagtgcatacaggtgaacggccctttccctgcacgtggccagactgccttaaaaagttctcccgctcagacgagctgacccgccactaccggacccacactggggaaaagcagttccgctgtccgctgtgtgagaagcgcttcatgaggagtgaccacctcacaaagcacgcccggcggcacaccgagttccaccccagcatgatcaagcgatcgaaaaaggcgctggccaacgctttgtgaggtgctgcccgtggaagccagggagggatggaccccgaaaggacaaaagtactcccaggaaacagacgcgtgaaaactgagccccagaagaggcacacttgacggcacaggaagtcactgctctttggtcaatattctgattttcctctccctgcattgtttttaaaaagcacattgtagcctaagatcaaagtcaacaacactcggtccccttgaagaggcaactctctgaacccgtctctgactgttggagggaaggcaaatgcttttgggttttttggtttttgtttttgtttttttttctccttttatttttttgcgggggagggtagggagtgggtgggggggaggggggtaaggccaagactggggtagaattttaaagattcaacactggtgtacatatgtccgctgggtgagttgacctgtggcctcgcacagtgattctgggccctttatgcttgctgtctctcagaattgttttcttaccttttaatgtaatgacgagtgtgcttcagtttgtttagcaaaaccactctcttgaatcacgttaacttttgagattaaaaaaaaaaacgccatagcacagctgtctttatgcaagcaagagcacatctactccagcatgatctgtcatctaaagacttgaaaacaaaaaacagttacttatagtcaatgggtaagcagagtctgaatttatactaatcaagacaaacctttgaaaggttacactaagtacagaacttttaaaccttgctttgtatgagttgtactttttgaacataagctgcacttttattttctaatgcagaggatgaataagttaaatacatgctttgaggatagaagcagatgttctgtttggcaccacgttataatctgcttattttacaatatacacgtttccctaagaaatcatggcagagatgtgagggcagaatatacacaacagatgctgaaggagaaggagggtagtgttttgcaaaagaaaaagaaaagaaccaacagaattttaactctattaacttttccaaattttcctatgcttttagttaacatcattattgtatcctaatgccactaggggagagagcttttgactctgttgggttttatttgaatgtgtgcataacagtaatgagatctggaaacacctattttttggggaaaaaggtttgttggtctccttcctgtgttcctacaaaactcccactctcaggtgcaagagttatgtagaaggaaagggagctgaaataggaacagaaaaatcaacccctataactagtgaacaccaagggaaaataccacaatgatttcagaggagactctgcaaaatcgtcccttgtggagaatgcaggcaacatggaatactaggaatgaaatcacatcactgtatcttttacatcaatagcctcaccactaatatatcttgtatctaggtgtctataatggctgaaaccactacatccatctatgccatttacctgaaaacttaactgtggcctttatgaggccagaaaagtgaactgagttttcgtagttaagacctcaaatgaggggagtcagcagtgatcatgggggaaatgtttacattttttttttcttcagaagtaacgctttctgatgattttatctgatatttaaaacagggagctatggtgcactctagtttatacttgcgctctgaaatgtgtaaacatagggtgcctacctatttcacctgacccatactcgtttctgattcagaatcagtgtgggctcctgcagtgggcgcgggtcacggctgactccaacttccaatacaacagccatcactagcacagtgtttttttgtttaaccaacgtagttgtattagtagttctataaagagaactgcttttaacattagggactgggagcagtccatgggataaaaaggaaagtgttttctcacgagaaaacatgtcaggaaaaataaagaacactttctacctctgtttcagatttttgaaacacttattttaaaccaaattttaatttctgtgtccaaaataagttttaaggacatctgttcttccatacgaaataggttaggctgcctatttctcactgagctcatggaatggttctgcttatgatactctgcacgctgccttttagtgagtgaggagtttggggttgcctagcaacttgctaacttgtaaaaagtcatctttccctcacagaaagaaacgaaagaaagcaaagcaaagtcagtgaaagacaatctttatagtttcaggagtaaatctaaatgtggcttttgtcaagcacttagatggatataaatgcagcaacttgttttaaaaaaatgcacaatttacttcccaaaaaagttgttacttgccttttcaagttgttgacaaacacacatttgatattctcttatatgttatagtaatgtaacgtataaactcaagcctttttattctttgtgattaaatcctgttttaaaatgtcacaaaacaggaaccagcattctaattagatttactatatcaagatatggttcaaataggactactagagttcattgaacactaaaactatgaaacaattactttttatattaaaaagaccatggatttaacttatgaaaatccaaatgcaggatagtaatttttgtttacttttttaaccaaactgaatttttgaaagactattgcaggtgtttaaaaagaaagaaaagttgttttatctaatactgtaagtagttgtcatattctggaaaatttaatagttttagagttaagatatctcctctctttggttagggaagaagaaagcccttcaccattgtggaatgatgccctggctttaaggtttagctccacatcatgcttctcttgagaattctatttggtagttacaattacagaaactgattagtttgtcagtttgcagatagatttagcacagtactcatcactcggatagattgagatgttctttcacatcagatgatctgtaacactgtaagatactgatctttacaactgtttaatcagttttatttttgtacagtattagtgacctaagttattttgctgtcccgtttttgtaaatcaaatgaaattataaaagaggattctgacagtaggtattttgtacatatgtatatatgttgtccaaataaaaataataaatgataaagactgaa

SEQ ID NO: 21 - DNA, 2303 bp, Homo sapiens
gaattcgtccaaactgaggatcacaagtctccacattctgagtaggaggatgagggtctgagttaggatttgggtcctgcagggcttgctaaggaatcccctgatggcctaggattccacgcagagcacatctggtgtgagagagctcgctgcaagggtgaaggctccgccctatcagatagacaaccaggccaccaagaggcccagccctccaaaccctggatttgcaacatcctcaaagaacagcaacgggccttgagcagaattgagaaggaaatacccccacctgccctcagccgttaagtgggctttgctattcacaagggcctctgggtgtcctggcagagaggggagatggcacaggcaccaggtgctagggtgccagggcctcccgagaaggaacaggtgcaaagcaggcaattagcccagaaggtatccgtggggcaggcagcctagatctgatgggggaagccaccaggattacatcatctgctgtaacaactgctctgaaaagaagatatttttcaacctgaacttgcagtagctagtggagaggcaggaaaaaggaaatgaaaccagagacagagggaagctgagcgaaaatagaccttcccgagagaggaggaagcccggagagagacgcacggtcccctccccgcccctaggccgccgccccctctctgccctcggcggcgagcagcgcgccgcgacccgggccgaaggtgcgaggggctccgggcggccgggcgggcgcacaccatccccgcgggcggcgcggagccggcgacagcgcgcgagagggaccgggcggtggcggcggcgggaccgggatggaagggagcgcggtgactgtccttgagcgcggaggggcgagctcgccggcggagcgccggagcaagcggaggcgcaggagcggcggcgacggcggcggcggcggcggcgcccgagcacccgagggggtccgagccccggcagccggccagccccgcgccacaaagggagcgcccccgccgcccggcaccccgcctccctccccaatgtcctcggccatcgaaaggaagagcctggacccttcagaggaaccagtggatgaggtgctgcagatccccccatccctgctgacatgcggcggctgccagcagaacattggggaccgctacttcctgaaggccatcgaccagtactggcacgaggactgcctgagctgcgacctctgtggctgccggctgggtgaggtggggcggcgcctctactacaaactgggccggaagctctgccggagagactatctcaggctttttgggcaagacggtctctgcgcatcctgtgacaagcggattcgtgcctatgagatgacaatgcgggtgaaagacaaagtgtatcacctggaatgtttcaaatgcgccgcctgtcagaagcatttctgtgtaggtgacagatacctcctcatcaactctgacatagtgtgcgaacaggacatctacgagtggactaagatcaatgggatgatataggcccgagtccccgggcatctttggggaggtgttcactgaagacgccgtctccatggcatcttcgtcttcactcttaggcactttgggggtttgagggtggggtaagggatttcttaggggatggtagacctttattgggtatcaagacatagcatccaagtggcataattcaggggctgacacttcaaggtgacagaaggaccagcccttgagggagaacttatggccacagcccatccatagtaactgacatgattagcagaagaaaggaacatttaggggcaagcaggcgctgtgctatcatgatggaatttcatatctacagatagagagttgttgtgtacagacttgttgtgactttgacgcttgcgaactagagatgtgcaattgatttcttttcttcctggctttttaactcccctgtttcaatcactgtcctccacacaagggaaggacagaaaggagagtggccattctttttttcttggcccccttcccaaggccttaagctttggacccaaggaaaactgcatggagacgcatttcggttgagaatggaaaccacaacttttaaccaaacaattatttaaagcaatgctgatgaatcactgtttttagacaccttcattttgaggggaggagttccacagattgtttctatacaaatataaatcttaaaaagttgttcaactattttattatcctagattatatcaaagtatttgtcgtgtgtagaaaaaaaaacagctctgcaggcttaataaaaatgacagactgaaaaaaaaaaaa

SEQ ID NO: 22 - DNA, 3198 bp, Homo sapiens
atttgaggtgttctgaccagaagaagacagagcggatgatcattcattcaccacgttgacaacctcgcctgtgattgacagctggagtggcagaaagccatgagatttggtagttgggtctgaggggcgctcttttttttccttttctttctttctttctttttttttttttaaactgatttttgggggagagaagatctgcttttttttgcccccgctgctgtcttggaaacggagcgcttttatgctcagtgactcgggcgctttgcttcaggtcccgtagaccgaagatctgggaccagtagctcacgttgctggagacgttaagggatttttcgtcgtgcttttttttttttttttttttttttccgggggagtttgaatatttgtttcttttcacactggccttaaagaggatatattagaagttgaagtaggaagggagccagagaggccgatggcgcaaaggtacgacgatctaccccattacgggggcatggatggagtaggcatcccctccacgatgtatggggacccgcatgcagccaggtccatgcagccggtccaccacctgaaccacgggcctcctctgcactcgcatcagtacccgcacacagctcataccaacgccatggcccccagcatgggctcctctgtcaatgacgctttaaagagagataaagatgccatttatggacaccccctcttccctctcttagcactgatttttgagaaatgtgaattagctacttgtaccccccgcgagccgggggtggcgggcggggacgtctgctcgtcagagtcattcaatgaagatatagccgtgttcgccaaacagattcgcgcagaaaaacctctattttcttctaatccagaactggataacttgatgattcaagccatacaagtattaaggtttcatctattggaattagagaaggtacacgaattatgtgacaatttctgccaccggtatattagctgtttgaaagggaaaatgcctatcgatttggtgatagacgatagagaaggaggatcaaaatcagacagtgaagatataacaagatcagcaaatctaactgaccagccctcttggaacagagatcatgatgacacggcatctactcgttcaggaggaaccccaggcccttccagcggtggccacacgtcacacagtggggacaacagcagtgagcaaggtgatggcttggacaacagtgtagcttcccccagcacaggtgacgatgatgaccctgataaggacaaaaagcgtcacaaaaagcgtggcatctttcccaaagtagccacaaatatcatgagggcgtggctgttccagcatctaacacacccttacccttctgaagaacagaaaaagcagttggcacaagacacgggactcaccatccttcaagtgaacaattggtttattaatgcccggagaagaatagtgcagcccatgatagaccagtccaaccgagcagtaagtcaaggaacaccttataatcctgatggacagcccatgggaggtttcgtaatggacggtcagcaacatatgggaattagagcaccaggacctatgagtggaatgggcatgaatatgggcatggaggggcagtggcactacatgtaaccttcatctagttaaccaatcgcaaagcaagggggaaggctgcaaagtatgccaggggagtatgtagcccggggtggtccaatgggtgtgagtatgggacagccaagttatacccaaccccagatgcccccccatcctgctcagctgcgtcatgggccccccatgcatacgtacattcctggacaccctcaccacccaacagtgatgatgcatggaggaccgccccaccctggaatgccaatgtcagcatcaagccccacagttcttaatacaggagacccaacaatgagtggacaagtcatggacattcatgctcagtagcttaagggaatatgcattgtctgcaatggtgactgatttcaaatcatgttttttctgcaatgactgtggagttccattcttggcatctactctggaccaaggagcatccctaattcttcatagggacctttaaaaagcaggaaataccaactgaagtcaatttgggggacatgctaaataactatataagacattaagagaacaaagagtgaaatattgtaaatgctattatactgttatccatattacgttgtttcttatagattttttaaaaaaaatgtgaaatttttccacactatgtgtgttgtttccatagctcttcacttcctccagaagcctccttacattaaaaagccttacagttatcctgcaagggacaggaaggtctgatttgcaggatttttagagcattaaaataactatcaggcagaagaatctttcttctcgcctaggatttcagccatgcgcgcgctctctctctttctctctcttttcctctctctccctctttctagcctggggcttgaatttgcatgtctaattcatttactcaccatatttgaattggcctgaacagatgtaaatcgggaaggatgggaaaaactgcagtcatcaacaatgattaatcagctgttgcaggcagtgtcttaaggagactggtaggaggaggcatggaaaccaaaaggccgtgtgtttagaagcctaattgtcacatcaagcatcattgtccccatgcaacaaccaccaccttatacatcacttcctgttttaagcagctctaaaacatagactgaagatttatttttaatatgttgactttatttctgagcaaagcatcggtcatgtgtgtattttttcatagtcccaccttggagcatttatgtagacattgtaaataaattttgtgcaaaaaggactggaaaaatgaactgtattattgcaatttttttttgtaaaagtagcagtttggtatgagttggcatgcatacaagatttactaagtgggataagctaattatactttttgttgtggataaacaaatgcttgttgatagcctttttctatcaagaaaccaaggagctaattattaataacaatcattgcacactgagtcttagcgtttctgatggaaacagtttggattgtataataacgccaagcccagttgtagtcgtttgagtgcagtaatgaaatctgaatctaaaataaaaacaagattatttttgtcaaaaaaaaaaaaaaaaaa

SEQ ID NO: 23 - DNA, 1581 bp, Homo sapiens
ctcgccgctgccccggctccgccgctcgcagagagattcggaggagcccgggcgggggggaggaggagggggaggagggagcggagatctcggggctcggagccggccgccgctccgctccgatcgctgtggggcttggttttttgggggtgggggggcgggggggctcagatatggaggcaaatgggagccaaggcacctcgggcagcgccaacgactcccagcacgaccccggtaaaatgtttatcggtggactgagctggcagacctcaccagatagccttagagactattttagcaaatttggagaaattagagaatgtatggtcatgagagatcccactacgaaacgctccagaggcttcggtttcgtcacgttcgcagacccagcaagtgtagataaagtattaggtcagccccaccatgagttagattccaagacgattgaccccaaagttgcatttcctcgtcgagcgcaacccaagatggtcacaagaacaaagaaaatatttgtaggcgggttatctgcgaacacagtagtggaagatgtaaagcaatatttcgagcagtttggcaaggtggaagatgcaatgctgatgtttgataaaactaccaacaggcacagagggtttggctttgtcacttttgagaatgaagatgttgtggagaaagtctgtgagattcatttccatgaaatcaataataaaatggtagaatgtaagaaagctcagccgaaagaagtcatgttcccacctgggacaagaggccgggcccggggactgccttacaccatggacgcgttcatgcttggcatggggatgctgggatatcccaacttcgtggcgacctatggccgtggctaccccggatttgctccaagctatggctatcagttcccaggcttcccagcagcggcttatggaccagtggcagcagcggcggtggcggcagcaagaggatcaggctccaacccggcgcggcccggaggcttcccgggggccaacagcccaggacctgtcgccgatctctacggccctgccagccaggactccggagtggggaattacataagtgcggccagcccacagccgggctcgggcttcggccacggcatagctggacctttgattgcaacggcctttacaaatggataccattgagcaggtgctttcgttgccatctcactctgagagcatacctggatgtccaggcaagactgggcgaagtttctgagtggccctttgtttaggtgatgtcctcagacctggacccccaccagcctcactccccatcccaaccagagatggctcacttcggatcgagggttgactacatctcatcatctcacgaatctgctgtaatataagacaacagcttttaaatgtgtatataacccatgatttcggttttgttttgttttgtttttcttgatggtttccctctccctccctctcttcccattctccttttaaatctctttgaatcacatttggtagtgattttgacttagtccagtagtcacatagctttaatatctagttcaaagctaaccatagtataattgttatattaaggagttat

SEQ ID NO: 24 - DNA, 2613 bp, Homo sapiens
gtcatctgtctggacgcgctgggtggatgcggggggctcctgggaactgtgttggagccgagcaagcgctagccaggcgcaagcgcgcacagactgtagccatccgaggacacccccgcccccccggcccacccggagacacccgcgcagaatcgcctccggatcccctgcagtcggcgggagtgttggaggtcggcgccggcccccgccttccgcgccccccacgggaaggaagcacccccggtattaaaacgaacggggcggaaagaagccctcagtcgccggccgggaggcgagccgatgccgagctgctccacgtccaccatgccgggcatgatctgcaagaacccagacctcgagtttgactcgctacagccctgcttctacccggacgaagatgacttctacttcggcggccccgactcgacccccccgggggaggacatctggaagaagtttgagctgctgcccacgcccccgctgtcgcccagccgtggcttcgcggagcacagctccgagcccccgagctgggtcacggagatgctgcttgagaacgagctgtggggcagcccggccgaggaggacgcgttcggcctggggggactgggtggcctcacccccaacccggtcatcctccaggactgcatgtggagcggcttctccgcccgcgagaagctggagcgcgccgtgagcgagaagctgcagcacggccgcgggccgccaaccgccggttccaccgcccagtccccgggagccggcgccgccagccctgcgggtcgcgggcacggcggggctgcgggagccggccgcgccggggccgccctgcccgccgagctcgcccacccggccgccgagtgcgtggatcccgccgtggtcttcccctttcccgtgaacaagcgcgagccagcgcccgtgcccgcagccccggccagtgccccggcggcgggccctgcggtcgcctcgggggcgggtattgccgccccagccggggccccgggggtcgcccctccgcgcccaggcggccgccagaccagcggcggcgaccacaaggccctcagtacctccggagaggacaccctgagcgattcagatgatgaagatgatgaagaggaagatgaagaggaagaaatcgacgtggtcactgtggagaagcggcgttcctcctccaacaccaaggctgtcaccacattcaccatcactgtgcgtcccaagaacgcagccctgggtcccgggagggctcagtccagcgagctgatcctcaaacgatgccttcccatccaccagcagcacaactatgccgccccctctccctacgtggagagtgaggatgcacccccacagaagaagataaagagcgaggcgtccccacgtccgctcaagagtgtcatccccccaaaggctaagagcttgagcccccgaaactctgactcggaggacagtgagcgtcgcagaaaccacaacatcctggagcgccagcgccgcaacgaccttcggtccagctttctcacgctcagggaccacgtgccggagttggtaaagaatgagaaggccgccaaggtggtcattttgaaaaaggccactgagtatgtccactccctccaggccgaggagcaccagcttttgctggaaaaggaaaaattgcaggcaagacagcagcagttgctaaagaaaattgaacacgctcggacttgctagacgcttctcaaaactggacagtcactgccactttgcacattttgattttttttttaaacaaacattgtgttgacattaagaatgttggtttactttcaaatcggtcccctgtcgagttcggctctgggtgggcagtaggaccaccagtgtggggttctgctgggaccttggagagcctgcatcccaggatgctgggtggccctgcagcctcctccacctcacctccatgacagcgctaaacgttggtgacggttgggagcctctggggctgttgaagtcaccttgtgtgttccaagtttccaaacaacagaaagtcattccttctttttaaaatggtgcttaagttccagcagatgccacataaggggtttgccatttgatacccctggggaacatttctgtaaataccattgacacatccgccttttgtatacatcctgggtaatgagaggtggcttttgcggccagtattagactggaagttcatacctaagtactgtaataatacctcaatgtttgaggagcatgttttgtatacaaatatattgttaatctctgttatgtactgtactaattcttacactgcctgtatactttagtatgacgctgatacataactaaatttgatacttatattttcgtatgaaaatgagttgtgaaagttttgagtagatattactttatcactttttgaactaagaaacttttgtaaagaaatttactatatatatatgcctttttcctagcctgtttcttcctgttaatgtatttgttcatgtttggtgcatagaactgggtaaatgcaaagttctgtgtttaatttcttcaaaatgtatatatttagtgctgcatcttatagcactttgaaatacctcatgtttatgaaaataaatagcttaaaattaaatgaaaaaaaaa

SEQ ID NO: 25 - DNA, 2761 bp, Homo sapiens
aaatgaggagcgaactaaaggcacactgggaacgaaattaacgggaggtctgactgcaaggggagggggctcgcgatctaaaacgagaagagatctcggggtctcatactgcgccattcggctgcggtacatctcggcactctagctgcagccgggagaggccttgccgccaccgctgtcgcccaagcctccactgccgctgccacctcagcgccggcctctgcatccccagctccagctccgctctgcgccgctgctgccatcgccgctgccacctccgcagcccgggcctccgccgccgccactcaagcatccgtgagtcattttctgcccatctctggtcgcgcggtctccctggtagagtttgtaggcttgcaagatggcagaagcagattttaaaatggtctcggaacctgtcgcccatggggttgccgaagaggagatggctagctcgactagtgattctggggaagaatctgacagcagtagctctagcagcagcactagtgacagcagcagcagcagcagcactagtggcagcagcagcggcagcggcagcagcagcagcagcagcggcagcactagcagccgcagccgcttgtatagaaagaagagggtacctgagccttccagaagggcgcggcgggccccgttgggaacaaatttcgtggataggctgcctcaggcagttagaaatcgtgtgcaagcgcttagaaacattcaagatgaatgtgacaaggtagataccctgttcttaaaagcaattcatgatcttgaaagaaaatatgctgaactcaacaagcctctgtatgataggcggtttcaaatcatcaatgcagaatacgagcctacagaagaagaatgtgaatggaattcagaggatgaggagttcagcagtgatgaggaggtgcaggataacacccctagtgaaatgcctcccttagagggtgaggaagaagaaaaccctaaagaaaacccagaggtgaaagctgaagagaaggaagttcctaaagaaattcctgaggtgaaggatgaagaaaaggaagttcctaaagaaattcctgaggtaaaggctgaagaaaaagcagattctaaagactgtatggaggcaacccctgaagtaaaagaagatcctaaagaagtcccccaggtaaaggcagatgataaagaacagcctaaagcaacagaggctaaggcaagggctgcagtaagagagactcataaaagagttcctgaggaaaggcttcaggacagtgtagatcttaaaagagctaggaagggaaagcctaaaagagaagaccctaaaggcattcctgactattggctgattgttttaaagaatgttgacaagctcgggcctatgattcagaagtatgatgagcccattctgaagttcttgtcggatgttagcctgaagttctcaaaacctggccagcctgtaagttacacctttgaatttcattttctacccaacccatacttcagaaatgaggtgctggtgaagacatatataataaaggcaaaaccagatcacaatgatcccttcttttcttggggatgggaaattgaagattgcaaaggctgcaagatagactggagaagaggaaaagatgttactgtgacaactacccagagtcgcacaactgctactggagaaattgaaatccagccaagagtggttcctaatgcatcattcttcaacttctttagtcctcctgagattcctatgattgggaagctggaaccacgagaagatgctatcctggatgaggactttgaaattgggcagattttacatgataatgtcatcctgaaatcaatctattactatactggagaagtcaatggtacctactatcaatttggcaaacattatggaaacaagaaatacagaaaataagtcaatctgaaagatttttcaagaatcttaaaatctcaagaagtgaagcagattcatacagccttgaaaaaagtaaaaccctgacctgtaacctgaacactattattccttatagtcaagtttttgtggtttcttggtagtctatattttaaaaatagtcctaaaaagtgtctaagtgccagtttattctatctaggctgttgtagtataatattcttcaaaatatgtaagctgttgtcaattatctaaagcatgttagtttggtgctacacagtgttgatttttgtgatgtcctttggtcatgtttctgttagactgtagctgtgaaactgtcagaattgttaactgaaacaaatatttgcttgaaaaaaaaagttcatgaagtaccaatgcaagtgttttatttttttcttttttccagcccataagactaagggtttaaatctgcttgcactagctgtgccttcattagtttgctatagaaatccagtacttatagtaaataaaacagtgtattttgaagtttgactgcttgaaaaagattagcatacatctaatgtgaaaagaccacatttgattcaactgagaccttgtgtatgtgacatatagtggcctataaatttaatcataatgatgttattgtttaccactgaggtgttaatataacatagtatttttgaaaaagtttcttcatcttatattgtgtaattgtaaactaaagataccgtgttttctttgtattgtgttctaccttccctttcactgaaaatgatcacttcatttgatactgtttttcatgttcttgtattgcaacctaaaataaataaatattaaagtgtgttatactataaaaatctaaaa

SEQ ID NO: 26 - DNA, 2761 bp, Homo sapiens; identical to SEQ ID NO: 25

SEQ ID NO: 27 - DNA, 5545 bp, Homo sapiens
aactcttttgagtccagaatctcagaatcgggcgttgggctttgccgggtgcttcagatcaatggatgagttccacccgttcatcgaggcactgctgcctcacgtccgcgctttctcctacacctggttcaacctgcaggcgcggaagcgcaagtacttcaagaagcatgaaaagcggatgtcgaaggacgaggagcgggcggtgaaggacgagctgctgggcgagaagcccgagatcaagcagaagtgggcatcccggctgctggccaagctgcgcaaggacatccggcccgagttccgcgaggacttcgtgctgaccatcacgggcaagaagcccccctgctgcgtgctctccaaccccgaccagaagggcaagatccggcggattgactgcctgcgccaggctgacaaggtgtggcggctggacctggtcatggtgattttgtttaaggggatccccctggaaagtactgatggggagcggctctacaagtcgcctcagtgctcgaaccccggcctgtgcgtccagccacatcacattggagtcacaatcaaagaactggatctttatctggcttactttgtccacactccggaatccggacaatcagatagttcaaaccagcaaggagatgcggacatcaaaccactgcccaacgggcacttaagtttccaggactgttttgtgacttccggggtctggaatgtgacggagctggtgagagtatcacagactcctgttgcaacagcatcagggcccaacttctccctggcggacctggagagtcccagctactacaacatcaaccaggtgaccctggggcggcggtccatcacctcccctccttccaccagcaccaccaagcgccccaagtccatcgatgacagtgagatggagagccctgttgatgacgtgttctatcccgggacaggccgttccccagcagctggcagcagccagtccagcgggtggcccaacgatgtggatgcaggcccggcttctctaaagaagtcaggaaagctggacttctgcagtgccctctcctctcagggcagctccccgcgcatggctttcacccaccacccgctgcctgtgcttgctggagtcagaccagggagcccccgggccacagcatcagccctgcacttcccctccacgtccatcatccagcagtcgagcccgtatttcacgcacccgaccatccgctaccaccaccaccacgggcaggactcactgaaggagtttgtgcagtttgtgtgctcggatggctcgggccaggccaccggacagcattcgcaacgacaggcgcctcctctgccaaccggtttgtcagcatcggaccccgggacggcaactttctgaacatcccacagcagtctcagtcctggttcctctgataagatcgacaaaagaaacaacaaaatgagaagaagaggttcctcgaaaggggggagaagaaattttgagaatggaaaaatcccccagcccagcccagccccaccgaaaagcaaaaattacacgtcgtcagccactcagcccttctctcctccagcccggggacccccgcgggccccagaagcagcccagttctcagagagcccttggaaggggtctcggtggagctgtgcaccagcagccaagcagaaagaaacacgcgacatggactctgtcaagtagaggacagaaagcaagaaaggatgcagaactgccttcctccccctgaccccgccccggccttctggggaaggaacaaagtccccaaacaaagcaaccagcacaattctgaaggggcctggcctccaccctcaccccttcctaggggaaccccaccctccacacagccggagctgccctagggagcctggagggccagcttgtaaagatgatggggtttagatccctcaggctctcccctccagactccgcccttccctccctccctccctccctccctctctgccaaggctccagcttcttcccccagctgctcccgaccaggagggggagagcagcctccacttaccccaccccacccttgggctaaaagcccccaggcgggcagggggtgacccctggagctagttgcgtgtcccagaatggagggtgttctgacaccccaccctgagccgcaagagcagtcctggggccctggacccctctgtacagtccgtaggaaaaagtcggaatgctctcgacggcctcgtcccagcctgggacaggccccctttcccctctctctgcaggccaggagggcctccttcctgccacgagggaggggagtcgggccccaggtcgcccccgcccccagccctgcatgcaggtgccctcgctccgccccatcagttcctgcccctgcccctcatgcagactgccctgctggggccgggccggagggtggagcagaaaggggaccccggagccgagcgaggaggaccaggcagccgccgctgccgcgctaagccaccacctgcgcttaggtaggcgtcctgctcgccgactttcagttccttgggagggtgttgggtgtcgtccttttcaaaagtgttttggagctttctgtgccccccgactttcccccgcctccccgccccccacgtggccacttttctctggattttagctgtaatgtctttactctttatttaggggtggggcattcattgtttgggtcttttgctgttggaatgggaactcctcctccatttgagcaacttgggaacaatttggtaacacaccacaggaagtagctctcccccccagccccctcctccctcaagggagggttggggggcctgtccagagggtcttcagaagcccccctgggagggaggggaggatgagcacgcccagctcccctccagggtgtgacttggcccctctggcttgtctttctgtgccttactcctcctcctgcgtctcccgttcctggccccttcttgagtccttgtgcctctctctttctctctctttcttaattgtatgaaaacacaaagcacaggtcaggatcctctgagagaaaatcaacattgcaccacgtaggggtgggctatgggctgtatttattgtgaatctagtttgtgaggctgtggccccgagctggcggagggagggaagaggagggagtgacgggaggggaggaggtcagcgacctggggccgtagcggcaggcgaacggtgcctgctacccagctggaagccacaaggtggctggctccaggggcggcttttgttggaagttgagtgaagccctccccctgtcctcagcgtgcagccctagaggaccccagggctgaggggcagtggatcctgcgggagtctcccggggcgtggggagtaaggccccgggggtggggggccgggtgggccgggcgtgacgcgcggtcaaagtgcaatgatttttcagttcggttggctaaacagggtcagagctgagagcgaagcagaaggggctccctgtccggcccacgtgccctttccctcgacgacagtcgagggctcgggctctgtgggactgtgggagctagggtctgcggggcgcctgcccgggcgaggtcggaagctgcaggccagctgggcccgggccggagcgtgcccggcggggctgcccgggcgggcagggggtgggggctgctcctttcccaagtggtgttgtgaggggcaatgagggcaacaggagatgtggggacgtgttaggagagaaaaaaaaaaaaacaaaaatatatatgggggaaattaacttttttttttcattgaaccaagtgcaatgcatcagagagttttcctatctttgtatgttaagagattaagaaaaaaaaattctatttttgttgtaatgtcctcgcggctctggggacgctaaaagaaccgggcctgccccgccctgcgcggggataacgaaagctgagtgtttttcccttttttttgttcgtttttagtttttttttttttaagtcgttttcctgcgttgacgaggatgatctggggtttttatttgtttcgtcgttcgttctgtttcggtgggagggctgaaggaaacgttcacattttagagtttaaaaaaaacacctcgacatttaaaaaatcaaccaacacaagatcaaaaaggaaaaggacgagagaaaaattatttttaagataattaaacataaaaccctggtgcttcttacattataaagtacgttttaaagaacccacaaactattatacataagtttatgaatcaattaaatatcctgcacttgttaggaatacgcatatcccttctttgttgagtttaacggaacgggacagcggcgtgcccccggcggctggactgctccggccgcgggtctccccgggcgcccctccctggggcccagcacccctcctcgccccatccccgtccgggtacgggggcgcggcaggggtccccggcccctcccccgcagaggtcaatgccaacgaacaaacgtcccctccctccctccctctccgccccgagcgcccttctttgagccagacgccaacttgaccctcaccagcattatcaggagcgcgctcagcaagttggtagtttcctcccccctttcccggcgcccctcccgcccccattcaacatctctcatcctatccccgaccccctccggggaacaccgggaaggctcgacgctccaggacaggaccagccacgcagacaggtcgatttgcccaggcccgcgcccgcacgcacgcacgcacacggccccgcacacagccccgccccaccccgcaaccagccctgtcgactgccttatacacccgcccccgcgctggccggccgacctagtgccttgttctcacccccgtgctggcggagcggacgccgcgctctgggtcccagaggggccgggtggctcagacgacccaccactcccccaccctgaccgtgctgaacagacccccccacacgagagaaaataaaggagcaataaagtcacgagaactttcgtcccccaatcgagagcccgaggggcaccccagccccgcctctgctcccccccaccccacccaccctcggggcgcccccctccccccgcaagccagcctgggccagccccgcttcggcccctcccgggagatccgtgcgcccgaccagcaccagcatcgcggaccgcaaaggccgcccgtcccgtcaaacaagtttcttcttaggctaagaaacgcagtatatacgagtatctctatatatagtactaatggatttggtgtgcttcccccttagcgtccccctccctctgctcctcctccttcagcctggtctccccctcttctctgccctccacccccgtctctgcactgagatacataagaaacaagggtagtttactgtctgttttgttttctgggttttcagtgtcctagcggaatgcaagtaggcagccagcccgtctgttccctctccgccccgccccgccccgcccccgtcactgcgcttctgttataccatctttgcctgactctctccggcttctccattgaatggctaatgtgtatgtgaaataaagaaataaagaaaaacaaacgcga

SEQ ID NO: 28 - DNA, 2117 bp, Homo sapiens
agtccctgcagtggctgtaacaaaacccagacccccaggtcccggccaatggaggcgatttagactggagtgggaccgcgtctgtcaaaagcccgactcggcagcagcggcggagtccaggaggagagctggagccgccgcgctgcctccccgcccccgccgggatttattatttggactggacaattaagtggccctgatgatgttaccaagcccggtcacctccacccctttctcagtcaaagacattttgaatctggagcagcagcaccagcacttccatggtgcgcacttgcaggcggacttggagcaccacttccactctgcgccctgcatgctggccgccgctgaggggacgcaattttctgacggaggggaggaggacgaggaagacgagggcgagaaattgtcctatttgaactcactagccgcagcagacggccacggggattcagggctgtgtccccagggctatgtccacacggtcctgcgagactcgtgcagcgagcccaaggaacatgaagaggagcccgaggtcgtgagggaccggagccaaaaaagctgccagctgaagaagtctctagagacggccggagactgcaaggcggcggaggagagcgagaggccgaagccacgcagccgccggaagccccgggtcctcttctcgcaagcccaggtcttcgagctggaacgcaggttcaagcagcagcggtacctgtcggcacccgagcgcgagcacctcgccagcagcctgaagctcacatccactcaggtgaaaatctggttccagaatcgcaggtacaagtgcaagagacagcggcaggacaagtctctggagcttggcgcacacgcgcccccgccgccgccgcgccgcgtggctgtcccggtgctggtgcgggacggcaagccgtgcgtcacgcccagcgcgcaggcctacggcgcgccctacagcgtgggcgccagcgcctactcctacaacagcttccccgcctacggctatgggaactcggccgcggccgccgccgccgccgccgccgccgccgcagcagcggcggcctacagcagcagctatggctgtgcgtacccggcgggcggcggcggcggcggcggcgggacctccgcggcgaccactgccatgcagcccgcctgcagcgcggccggaggcggcccctttgtgaacgtgagcaacctaggaggcttcggcagcggcggcagcgcacagccgttgcaccagggtactgcagccggggccgcgtgcgctcagggcaccttgcagggcatccgggcctggtagggacggggcgggtcacgcggcgggcaccccagcgcagcctggcgccgcgggactgaagctcgagaagggcctgacctaaaggtcaggtcccctcgttaaaaaaatatgtacgtctagctcctcagggcttcggatcgcagctcactcgaggcctggggaaggggactcaggggcgaggaggatgactgggtccggtcgccaggactgtctctgaggcagaaacgccggctgggcgccggggaggacgatggccccgaccctggcagcgagaggagaccaggaggctaggaccctggccgcgcttggttcttccaaagcgagaagggcttctctccctctgcctttccgcggcctccgcgaagcgttggcggggagcccaaggacataacaaattaaaagcatgaaggagagaaaaatgggggtcgtggcttgagaaattccaggccctaccgatcctctgccccctttgcgggcctggagcgccatagcacagtcgatttcgtttcgcagctgtctcccctccgcagcagatacctcggtccagatctccggattgtcgggggacgcaggactcttcgaggaaaaccagccgaatgagatcaaaagttgggggtggggggaggctgaacaaactcaggacctggtggcccaccggaggtgttaccgggtttcctttctgtttcgtattctgtattcagcacatgttatctatctatctatctatataactataaccacacgccgtgtagacacccgctgccacacactacaggagtcaataaacaaggtgcaatattttcaaaaaaaaaaaaaaaaaaaaaaa

SEQ ID NO: 29 - DNA, 5915 bp, Homo sapiens
aaaagggccagccctccagctggcccactccccctccgggctttcgcccgccctctctccctcccttttttgcccgccctggccctgcccctgcccctgccccctcctctcagcccctccgcgcccggggtgtcattgggcccgggagacgggagccaacttcaggctgctcagaggaagcccgtgcagtcagtcacctgggtgcaagagcgttgctgcctcgggctctcccgctgcagggagagcggcactcgctggcctggatgtggttggatttaggggggctccgcagcaggggtttcgtggcggtggcaagcgctgcaacaggtagacggcgagagacggaccccggccgaggcagcgatggagaccaaaggctaccacagtctccctgaaggtctagatatggaaagacggtggggtcaagtttctcaggctgtggagcgttcttccctgggacctacagagaggaccgatgagaataactacatggagattgtcaacgtaagctgtgtttccggtgctattccaaacaacagtactcaaggaagcagcaaagaaaaacaagaactactcccttgccttcagcaagacaataatcggcctgggattttaacatctgatattaaaactgagctggaatctaaggaactttcagcaactgtagctgagtccatgggtttatatatggattctgtaagagatgctgactattcctatgagcagcagaaccaacaaggaagcatgagtccagctaagatttatcagaatgttgaacagctggtgaaattttacaaaggaaatggccatcgtccttccactctaagttgtgtgaacacgcccttgagatcatttatgtctgactctgggagctccgtgaatggtggcgtcatgcgcgccgttgttaaaagccctatcatgtgtcatgagaaaagcccgtctgtttgcagccctctgaacatgacatcttcggtttgcagccctgctggaatcaactctgtgtcctccaccacagccagctttggcagttttccagtgcacagcccaatcacccagggaactcctctgacatgctcccctaatgttgaaaatcgaggctccaggtcgcacagccctgcacatgctagcaatgtgggctctcctctctcaagtccgttaagtagcatgaaatcctcaatttccagccctccaagtcactgcagtgtaaaatctccagtctccagtcccaataatgtcactctgagatcctctgtgtctagccctgcaaatattaacaactcaaggtgctctgtttccagcccttcgaacactaataacagatccacgctttccagtccggcagccagtactgtgggatctatctgtagccctgtaaacaatgccttcagctacactgcttctggcacctctgctggatccagtacattgcgggatgtggttcccagtccagacacgcaggagaaaggtgctcaagaggtcccttttcctaagactgaggaagtagagagtgccatctcaaatggtgtgactggccagcttaatattgtccagtacataaaaccagaaccagatggagcttttagcagctcatgtctaggaggaaatagcaaaataaattcggattcttcattctcagtaccaataaagcaagaatcaaccaagcattcatgttcaggcacctcttttaaagggaatccaacagtaaacccgtttccatttatggatggctcgtatttttcctttatggatgataaagactattattccctatcaggaattttaggaccacctgtgcccggctttgatggtaactgtgaaggcagcggattcccagtgggtattaaacaagaaccagatgatgggagctattacccagaggccagcatcccttcctctgctattgttggggtgaattcaggtggacagtccttccactacaggattggtgctcaaggtacaatatctttatcacgatcggctagagaccaatctttccaacacctgagttcctttcctcctgtcaatactttagtggagtcatggaaatcacacggcgacctgtcgtctagaagaagtgatgggtatccggtcttagaatacattccagaaaatgtatcaagctctactttacgaagtgtttctactggatcttcaagaccttcaaaaatatgtttggtgtgtggggatgaggcttcaggatgccattatggggtagtcacctgtggcagctgcaaagttttcttcaaaagagcagtggaagggcaacacaactatttatgtgctggaagaaatgattgcatcattgataagattcgacgaaagaattgtcctgcttgcagacttcagaaatgtcttcaagctggaatgaatttaggagcacgaaagtcaaagaagttgggaaagttaaaagggattcacgaggagcagccacagcagcagcagcccccacccccacccccacccccgcaaagcccagaggaagggacaacgtacatcgctcctgcaaaagaaccctcggtcaacacagcactggttcctcagctctccacaatctcacgagcgctcacaccttcccccgttatggtccttgaaaacattgaacctgaaattgtatatgcaggctatgacagctcaaaaccagatacagccgaaaatctgctctccacgctcaaccgcttagcaggcaaacagatgatccaagtcgtgaagtgggcaaaggtacttccaggatttaaaaacttgcctcttgaggaccaaattaccctaatccagtattcttggatgtgtctatcatcatttgccttgagctggagatcgtacaaacatacgaacagccaatttctctattttgcaccagacctagtctttaatgaagagaagatgcatcagtctgccatgtatgaactatgccaggggatgcaccaaatcagccttcagttcgttcgactgcagctcacctttgaagaatacaccatcatgaaagttttgctgctactaagcacaattccaaaggatggcctcaaaagccaggctgcatttgaagaaatgaggacaaattacatcaaagaactgaggaagatggtaactaagtgtcccaacaattctgggcagagctggcagaggttctaccaactgaccaagctgctggactccatgcatgacctggtgagcgacctgctggaattctgcttctacaccttccgagagtcccatgcgctgaaggtagagttccccgcaatgctggtggagatcatcagcgaccagctgcccaaggtggagtcggggaacgccaagccgctctacttccaccggaagtgactgcccgctgcccagaagaactttgccttaagtttccctgtgttgttccacacccagaaggacccaagaaaacctgtttttaacatgtgatggttgattcacacttgttcaacagtttctcaagtttaaagtcatgtcagaggtttggagccgggaaagctgtttttccgtggatttggcgagaccagagcagtctgaaggattccccacctccaatcccccagcgcttagaaacatgttcctgttcctcgggatgaaaagccatatctagtcaataactctgattttgatattttcacagatggaagaagttttaactatgccgtgtagtttctggtatcgttcgcttgttttaaaagggttcaaggactaacgaacgttttaaagcttacccttggtttgcacataaaacgtatagtcaatatggggcattaatattcttttgttattaaaaaaacacaaaaaaataataaaaaaatatatacagattcctgttgtgtaataacagaactcgtggcgtggggcagcagctgcctctgagccctcgctcgtccacggtcttctgcatcactggtatacacactcgttagcgtccatttcttatttaattagaatggataagatgatgttaaatgccttggtttgatttctagtatctattgtgttggctttacaaataattttttgcagtcttttgctgtgctgtacattactgtatgtataaattatgaaggacctgaaataaggtataaggatcttttgtaaatgagacacatacaaaaaaaatctttaatggttaataggatgaatgggaaagtatttttgaaagaattctattttgctggagactatttaagtactatctttgtctaaacaaggtaatttttttttgtaaagtgcaatgtcctgcatgcataatgaaccgtttacagtgtatttaagaaagggaaagctgtgccttttttagcttcatatctaatttaccattattttacagtctctgttgtaaataaccacactgaaacctcttcggttgtcttgaaacctttctactttttctgtactttttgttttgttcttggtctcccgcttggggcatttgtgggactccagcacgttttctggcttctgcttcatcctgctccatcggggaatgacacactgcggtgtctgcagctcctggaaggtgtcatttgacaacacatgtgggagaggaggtccttggagtgctgcagctttgggaaagctgcctcgtttcccttttcctctagaagcagaaccagctctacgagagtgagactgggaacttgatggctcagagagcatcttttcctcccattttagaaaatcagattttctcctgtgggaaaaaaaaattccatgcactctctctctgttaaagatcagctattcccttctgatcttggaaagaggttctgcactcctggaaccggtcacaggaacgcacagatcatggcaggatgcgctgggacggcccatcttggcaaggttcagtctgaatggcatggagaccgggagatagaggggttttagatttttaaaaggtaggttttaaaaataagttttatacataaacagttttggagaaaaattacagatcatataagcaagacagtggcactaaaatgtttaattcattaatctgtttgtttggcactgatgcaatgtatggcttttctcttgccccaaatcacaaacatatgtatctttggggaaactaacaatatgattgcactaaataaactactttgaatagaggccaaattaatcttttaaaaatgatgataatcatcaggtttactcagtgaaatcatattaattattttccaaaatctaaaagctgtagctggagaagcccatggccacgaggaagcagcaattaattagatcaacacttttctccagggttcaccatgcaggcaacattaccttgtctttcaaaagacacctgccttatgcaaggggaaacctgtgaaagctgcactcagagggaggagtttttcttacataatttgcaatttcaggaatttaatttataggcagatctttaaatacagtcaacttacggtgcacagtaatatgaaagccacactttgaaggtaataaatacacagcatgcagactgggagttgctagcaaacaaatggcttacttacaaaagcagcttttagttcagacttagtttttataaaatgggaattctgacttacttaaccaggtttgggatggagatggtctgcatcagctttttgtattaacaaagttactggctctttgtgtgtctccaggtaactttgcttgattaaacagcaaagccatattctaaattcactgttgaatgcctgtcccagtccaaattgtctgtctgctcttatttttgtaccatattgctcttaaaaatcttggtttggtacagttcataattcaccaaaagttcatataatttaaagaaacactaaattagtttaaaatgaagcaatttatatctttatgcaaaaacatatgtctgtctttgcaaaggactgtaagcagattacaataaatcctttactttaatcaaaaaaaaaaaaaaaaaa

SEQ ID NO: 30 - DNA, 6805 bp, Homo sapiens
aaagtttgcattgcaatccccctgccttcctctcctttctcccgatcaatgcatatttgcaaaaggattaagccacagatttaagcgccgggagcccatttctgccttgcaaaggagaccggactgaaaaacctaaagccagctctgatttcttttcgccaagtgggaaggtggtttatttttcttgctttttggagtcaacacccttccccaccagcccttatccccaccctcaccccgcaaccccttcacgccccctccccctccccctcctcatcctcccaccatcctctaaagaggcaaagggattttttttttcttttggtcttcttttttcccccttccctgtttatcctgaaaaggatttgaagacaagcttgaaggataaaaagccttggtgcttcccaggagccgagccgaggagcagaagaggaagagccgggggctgccgtagcctttggagatggacgagcagcccaggctgatgcattcccatgctggggtcgggatggccggacaccccggcctgtcccagcacttgcaggatggggccggagggaccgagggggagggcgggaggaagcaggacattggagacattttacagcaaattatgaccatcacagaccagagtttggatgaggcgcaggccagaaaacatgctttaaactgccacagaatgaagcctgccttgtttaatgtgttgtgtgaaatcaaagaaaaaacagttttgagtatccgaggagcccaggaggaggaacccacagacccccagctgatgcggctggacaacatgctgttagcggaaggcgtggcggggcctgagaagggcggagggtcggcggcagcggcggcagcggcggcggcttctggaggggcaggttcagacaactcagtggagcattcagattacagagccaaactctcacagatcagacaaatctaccatacggagctggagaaatacgagcaggcctgcaacgagttcaccacccacgtgatgaatctcctgcgagagcaaagccggaccaggcccatctccccaaaggagattgagcggatggtcagcatcatccaccgcaagttcagctccatccagatgcagctcaagcagagcacgtgcgaggcggtgatgatcctgcgttcccgatttctggatgcgcggcggaagagacggaatttcaacaagcaagcgacagaaatcctgaatgaatatttctattcccatctcagcaacccttaccccagtgaggaagccaaagaggagttagccaagaagtgtggcatcacagtctcccaggtatcaaactggtttggaaataagcgaatccggtacaagaagaacataggtaaatttcaagaggaagccaatatttatgctgccaaaacagctgtcactgctaccaatgtgtcagcccatggaagccaagctaactcgccctcaactcccaactcggctggtggatacccttcgccatgttatcagccagacaggaggatacagtgatggactcgcagccagtcagatgtacagtccgcagggcatcagtgctaatggaggttggcaggatgctactaccccttcatcagtgacctcccctacagaaggccctggcagtgttcactctgatacctccaactgatctcccagcaatcgcatcccggctgaccctgtgccccagttggggcaggggcaggagggagggtttctctcccaacgctgaagcggtcagactggaggtcgaagcaatcagcaaacacaataagagtctccttctcttctcttctttgggatgctatttcagccaatctggacacttctttatactctcttcccttttttttctgggtagaagccacccttccctgcctccagctgtcagcctggttttcgtcatcttccctgcccctgtgcctctgtcctagactcccggggtccccgccctctctcatatcactgaaggatattttcaacaattagaggaatttaaagaggaaaaaaattacaaagaaaataataaaagtgtttgtacgttttcatgctggtggtttgaggagccaaatttacctcactcgaatccctcactccctatgttaacaggcaatccttctctgtttctcttattactctcactacctcttagcaggaatactccacattgccctattcattccaggcctccctgcttcctcttgctcttcctccctggggacagtactgattggaacactttcctcctcttccttcctagccccagctattcactggggactgtcatagctgggattctaaaggtgccacatttttcagtttcatctccactaggttggttcccgggcaggaagtcaggcagcagggaaggacacgggaacagcaggtggagaattcctacagtctttcttaccctgctagcaatagctctcagtttcagaggcacagtctttggagaccattcagcactgagaaagcaatatttagaacctattgcaaaactgggcctgagttaggcatggtgatgaatgcatcagcaaggaatagaaagttcttatcgtgaaacccttcaacctcaactatgccttcatagacacacacgttcatgcacatgtaggcacatgtaccatctcacatcttcactttcccgagatgccatatacaattacctacattaataactgtagcactatgccttttgagcccgagagagggaattagtgactctaagtgaaggtcactgacacagagaagcagtatgtgtctggggcttccaggacctgcaggcccactagcgtgcacttaccagaatggcatacacaggacctgatcatgaggaagaccaggtttccagtgtaaactactcttgttcccaccacctctggagcactcagggagccccatacagtacttacaatgtctttaatggacttgattctgtttaattttttgttttatattaggcacactgtattaattttccaaaatgttataccacactatgttcttggtcctgacctattgctctggaggaaagagttgtataagaacgtggctcatgtgaacttttgctagcttcatttgaggacctgagaatcatggggaaagggaaggtaatgttttcattgaaatcatcacagtgatttttattccctgggaacacagcgtgtactaaaaatacatgagaaaatagcatgtatatgaaagctattctcaaaagtcacctgagctcaccatcttcatagccaaccctaccagttataaagatggcagctctatcacttgattaagtgggaggtggtcaaatattttggtgcctcattttcttcatctgtgagatgggaactgttatgcctggcttactaagagtcttgtgagagactgagaagttgattttgttcatatccaatctgtaaatgcgaagtcaggggaagtaatgtccctgaaataaacgggttcatgccatctagggacaataaatggttttcttgttgtaacttctggttaatatcagtaccttgatgtcatcaccgtgatgacaaagagaagagttattgttgatcttcttggttttggtctgtctcttttcttaggataaagaaaaacttccaaactagaaaaacaggccctggttcccttagtttgcacttgaacccaatatgttgccttgtacatacttggtccctgtcacattgactgcttgggaggcttccagggagaagtatgagaccctgaggggtgagaatgggcagctagcaagaacatggaaattctgcttggcactacagtcataaatagaaaacactgtgtgtgctcaggggagcaggggatgccactgaagaaactcaagggaatgtgtatttgaaggaaatgcaaaaactaagtatttagcaaaatgaaattatgccttgatgactaaaaggcactagaaaggttgtgtctactaacttcagccctaatcagaacagatgcctagaaggagcatttttgtgacaacttcatagtgattagaatcagtggagaactccatcttagtggcaggaatataatgaaactacccacgcaagaacatggttgaatcacatttgcttgacttagggcaaagtacgaaagagagacaaaagggttctcttggaaacaagaagagtgactccagatgtggcctgaataattgccatgttaagttaatgcaaaagatcagaacagggctacatttgcacaggcagtttctctccgggccgtagttttcactgatgatcacctttcacagcattttccccaaccagcatttcacttagtcttctctatacccagcacctcccccggcacccccggcaagcccactatcacttccgacttccaacgtggcatccgtgagatctgtccacattaggcgaagcaggagaacactgagagcagcaggatgggtttggaaagagcatgcctctggaaacacagcttcctgggaattcacatgaggccagtcctacagagagcaagatgcaccccaggatttcttcattttctaatagatgtgggagtgctccattttccccgacagcgaatttcccctgagaaacgatactagaccctgggtttgcccaccttgtaactcttccttatctcctccttttcatccctaatccatcctccctctggcatggaattgacgcccgtgcagtacatttgccaagtggcaccttctttcaatttatgttttattttgctatggtggtgattctttatttgctggttgtcttttctcacacatctttctctctgtctctctctttcctgctctttgtttttctgcccagaaaaacctgacttcgataccaaaaaagatgaaactacagaaactcaaatttaaaaaaaactttaaaagaaacaaaaaaatactcaacgattctttcagctttattaacattttccattgtttcttgcgacttgtgtctcgttctttgtagtattgatgatgaacatttgataatgaatgttcttgtatattcagataaagaaaaaaaaaaccaaaaaagcggtctgaatttaatagtgtttataataaaaattttaaaaatgaccctcatagcacgcaaaacaggatggggaatttcccctcttctttctgtgacaatgcgcatcattcctgcattagtttttaacaccagactacctacattcatcatttccctcatttttcttttattttcttgcatttgtgaattagttcaagaatgctagaaaagtgtcgagttgtgcacatccatttcttgtttcacaatgtttaaaagtgacagtaattcattttgtaaactaaaaaaaaaaaaaaaaaggttggaatagtgagcataataggtacaacctaacacattattatgtttattaactttgagacccagaaataaattcttttcttttcttgattcttgctcttaaaaatacaaaaaaaaaaatgttttgttttgtgttatttttggtttgtttattggggggctttttttaattgtcaggattatgatcttgctgtttttcttcaatatgtatacaaggtgatgtgaaaagatgacttgggcagaggagtaagaacaagtaggcttgttcttctactttgcttcagaattcagttaatgccaaaagcgaagatcaagcccatgttgatgtctcgttgctcacctgcatttccagagagtgtgacactcatgcagtccctgagaaaaataaaatcagggacatacttctccttttagccttttaaaaattcaaaaacgtttagtccaagggaactttttatgctatcaggaaaggtttttgctgtttttgattctgattatcacagccaagtactttgttttatttctccctaattaataactacattccatgaggcctcttccaaccaaagaggccttttcttccaggagagtcccgcaggagatgctggtatgatgggcaccattggttaagtaaactacatgcaggaagaagtccttggggccagtctgccagctgagtcctggttttggatgaagagttaatgagatattgggccaggctcaatgctgtagttttaatgctaagaggttacgtttacttcacagagtacacctcttagtaacctctgacttaggcagctgcttaaagcaaattgcaaaactggcttgatttggaatgtttttattagaggaaaaaagaaagccatattatctggaaaaaaattcattttaaataccatcattcaacaaattatgttcagaaagtggtcagaacttaagcaagaaaagtaaagaaagaatgcagaattgtggagcaatgctttaggaaatatttctacctgaacacttgtactcttgaagtcacaacaaaataatgatgagcttttcacatcacctttatggtttcaatccctagctcaaagcttcctggaatcttttattttttgtaaacttttttttcttttgttaaaataaataaaacattcaatgtttttctccttttctctcttattacttctttcctttggcattttcaatttgaaatgctttcctttggttgttggttttattctccccctacccctccccttttcttattattcagaatataaacctgcaaagctctgctctgttttggttttgaaagtttaagcttttctgcttctgtgagagcacaggcttctgtcccttttgattccaactgaacttttgtgttctctaatgatactaacacggtgtaggttttacagtctcctaatttgtactggtaatgcatattccaaataaatagtttcttttgttgcaaaaaaaaaaaaaaaaa

SEQ ID NO: 31 - DNA, 8674 bp, Homo sapiens
ctgacaatgctggggagatgaagatagtgtgtggctgcttctggactcaaggaggaggagagagattccgcgagccgacaccatgcgatccaaggcgagggcgaggaagctagccaaaagtgacggtgacgttgtaaataatatgtatgagcccaaccgggacctgctggccagccacagcgcggaggacgaggccgaggacagtgccatgtcgcccatccccgtggggccaccgtcccccttccccaccagcgaggacttcacccccaaggagggctcgccgtacgaggcccctgtctacattcctgaagacattccgatcccagcagacttcgagctccgagagtcctccatcccaggggctggcctgggggtctgggccaagaggaagatggaagccggggagaggctgggcccctgcgtggtggtgccccgggcggcggcaaaggagacagacttcggatgggagcaaatactgacggacgtggaagtgtcgccccaggaaggctgcatcacaaagatctccgaagacctgggcagtgagaagttctgcgtggatgcaaatcaggcgggggctggcagctggctcaagtacatccgtgtggcgtgctcctgcgatgaccagaacctcaccatgtgtcagatcagtgagcagatttactataaagtcattaaggacattgagccaggtgaggagctgctggtgcacgtgaaggaaggcgtctaccccctgggcacagtgccgcccggcctggacgaggagcccacgttccgctgtgacgagtgtgacgaactcttccagtccaagctggacctgcggcgccataagaagtacacgtgtggctcagtgggggctgcgctctacgagggcctggctgaggagctcaagcccgagggccttggcggtggcagcggccaagcccacgagtgcaaggactgcgagcggatgttccccaacaagtacagcctggagcagcacatggtcatccacacggaggagcgcgagtacaaatgcgaccagtgtcccaaggccttcaactggaagtccaacctcatccgccaccagatgtcccacgacagcggcaaacgcttcgaatgtgaaaactgcgtgaaggtgttcacggaccccagcaaccttcagcggcacatccgctcgcagcacgtgggcgctcgggcccacgcctgccccgactgcgggaagaccttcgccacgtcctccggcctcaagcagcacaagcatatccacagcacggtgaagcctttcatatgtgaggtctgccacaagtcctacacgcagttctccaacctgtgccggcacaagcggatgcacgccgactgccgcacgcagatcaagtgcaaggactgtggccagatgttcagcactacctcctccctcaacaagcaccggcgcttctgcgagggcaagaaccattacacgccgggcggcatctttgccccgggcctgcccttgacccccagccccatgatggacaaggcaaaaccctcccccagcctcaatcacgccagcctgggcttcaacgagtactttccctccaggccgcacccggggagcctgcccttctccacggcgcctcccacgttccccgcactcacccccggcttcccgggcatcttccctccatccttgtacccccggccgcctctgctacctcccacatcgctgctcaagagccccctgaaccacacccaggacgccaagctccccagtcccctggggaacccagccctgcccctggtctccgccgtcagcaacagcagccagggcacgacggcagctgcggggcccgaggagaagttcgagagccgcctggaggactcctgtgtggagaagctgaagaccaggagcagcgacatgtcggacggcagtgactttgaggacgtcaacaccaccacggggaccgacctggacacgaccacggggacgggctcggacctggacagcgacgtggacagcgaccctgacaaggacaagggcaagggcaagtccgccgagggccagcccaagtttgggggcggcttggcgcccccgggggccccgaacagcgtggccgaggtgcctgtcttctattcccagcactcattcttcccgccacccgacgagcagctgctgactgcaacgggcgccgccggggactccatcaaggccatcgcatccattgccgagaagtactttggccccggcttcatggggatgcaggagaagaagctgggctcgctcccctaccactcggcgttccccttccagttcctgcccaacttcccccactccctttaccccttcacggaccgagccctcgcccacaacttgctggtcaaggccgagccaaagtcaccccgggacgccctcaaggtgggcggccccagtgccgagtgcccctttgatctcaccaccaagcccaaagacgtgaagcccatcctgcccatgcccaagggcccctcggcccccgcatccggcgaggagcagccgctggacctgagcatcggcagccgggcccgtgccagccaaaacggcggcgggcgggagccccgcaagaaccacgtctatggggaacgcaagctgggcgccggcgaggggctgccccaggtgtgcccggcgcggatgccccagcagcccccgctccactacgccaagccctcgcccttcttcatggaccccatctacagcagggtagaaaagcggaaggtcacagaccccgtgggagccctgaaggagaagtacctgcggccgtccccgctgctcttccacccccagatgtcagccatagagaccatgacagagaagctggagagctttgcagccatgaaggcggactcgggcagctccctgcagcccctcccccaccaccccttcaacttccggtccccacccccaacgctctccgaccccatcctcaggaagggcaaggagcgatacacgtgcaggtactgtgggaagatcttccccagatcagccaatctcaccagacacctgaggacgcacactggggagcagccgtacaggtgtaagtactgcgaccgctccttcagcatctcttcgaacctccagcggcacgtccggaacatccacaacaaggagaagcctttcaagtgccacctgtgcaaccgctgcttcgggcagcagaccaacctggaccggcacctcaagaagcacgagcacgagaacgcaccagtgagccagcaccccggggtcctcacgaaccacctggggaccagcgcgtcctctcccacctcagagtcggacaaccacgcacttttagacgagaaagaagactcttatttctcggaaatcagaaactttattgccaatagtgagatgaaccaagcatcaacgcgaacagagaaacgggcggacatgcagatcgtggacggcagtgcccagtgtccaggcctagccagtgagaagcaggaggacgtggaggaggaggacgacgatgacctggaggaggacgatgaggacagcctggccgggaagtcgcaggatgacaccgtgtcccccgcacccgagccccaggccgcctacgaggatgaggaggatgaggagccagccgcctccctggccgtgggctttgaccacacccgaaggtgtgctgaggaccacgaaggcggtctgttagctttggagccgatgccgacttttgggaaggggctggacctccgcagagcagctgaggaagcatttgaagttaaagatgtgcttaattccaccttagattctgaggctttaaaacatacactgtgcaggcaggctaagaaccagggttctctggacgcttggttgaaggtcactggagccacgtcggagtctggagcatttcaccccatcaaccacctctgacgggctgggcagccgggggccggtggccagagcgagggcaccagccacgaaggacggaggcgggcggggccccggagaaccctgtccctgcgtgtggccactcctcagcatcctccccacccaccatggttcattccgacttttccaatggaaactcagatcccaaaagtccctaaagcagtcgtagagtctcaccatctccaaggattggtcttgagaacactgttcagtgacggccatgcaggtggccgtccaaagacagccaacggagctgcctcgcagaatcagccagtgggcaggtggacgctctgctgagacagaagctggtggccactgccgggtgcccgcgtggggtcgcggaagggaatggatagactggtgtgctcaaaagagagagatcactcaaatgatttttataatgaaatgacaagaataacccttttggtaaccgtattgactgcagagtctatttaagcatgtggttttaaaaatagacagtattttttaaaaatcaaaaaatgacttgcaaattgttttttaaaagtaattttgcattgctttgaaatttgagctcatttgcaaacccgagtctgcctgggaacccgcactgtgcctgggtgtattctttatactgtagataatggagaaattttctatctctgtccctatttgtataagccaaggtgatgctgggtgccccgaggcagaacaagaggcgcggggccacacccgtgaaccatgcagacggccgaagaagtcttaggcagggcgccctgggctgcaggcctgcccgaggctgggatgggaagtgtgcctgccctcgtgtgacatggaattggtgtcaggaccgccacgtggccttcagaggaatccacaggtccccacccaagatccctcaattatatggggaagtcgagggcctgtggcttggatccgccatgcagagatgtggccgggcacccatcttccttccctcctctgtccctgcctcggccaccccacgcgggaacccagcgccgtcctctgaaggcagggccttggccacgtcctgggtctcccacctcccacctgaccccagcggctccggtgtcctccacgtggctgccctggggagcaatcccagcggatcgctccgggccaccaagccgcacctgtgcctgagactccggatggacgacacagtcgtcacgtcgctcttcctgcgggttcttggcgagacacagcttgagaacagaagggcgtcgggggaacctgccgcaaggagcagagacagcacagccccccgggcccagccgcctccctctcttgggacgcaacttcttccccactcggatgggctttaaattattcccataggggccaatttcaaataataatttttttccctgatggaatttaccttaatctgtatataacttgtaattttttctaattcatttcttttcttattttatttcctccttaacagtatttttggcattagacattcttattgtgaagaaataatgttaatataagtatctggtgaaggaccaaaaccgtgtgataaggttgtgtgtcgtgtgggagtggggcgattttttatgtgccaaatacccccgtcccccccatgaatcctgctgtccctgctgccgtttaccagacaatcatatgtttttgttaaatttgcgtttcagttacatttgcatttaagacaagtgttctatttatttcttgtattgtttggaagaaaaaatgatgatagagtcccaaaaagaagagaaaaaaaatgcccaagttgccctttaaaaaaaaagagcgtaaatacaaacaggagtggtgcaagccgccttggtgtgggtttgtgtcacgtgtggacatctcctcaggctttgtgtcacgcgtggacatctcctcaggctgtccccagcggtgacgggaggtgtcctggctgctccaggacaaaagacaatcgtctctgtgggtgccgggtggtccaggcttgcactgaagacgtgccacggggaggctcctgcaggaggctcaacccgacggatcacagtgaaagggattcctcccacgccagatctgcacaacgaggcaagacaggacccacctgtgcgtgcgctggggccatggggtggccccgccggggcagcgggggagctgcctgcagaagagccagctggcgtgtcgggaaggatccaggatctgcaaacacaactgctcaggccttctcacgcgtttccacaacatcccctgggtcagacccaccaggtaccccgtaggaatttccagtttcccttgatctagatgggattcttataaaaattcaacctcagacataaacaccccatttctgtaaacccaaattatatggtttcttctgcgaaagagtaaggtgtgtgcttttttttttttgcaatatgaccccgtctctctgaagtgggacattcggacggatggagccctcagcgtgtcttttcagcaggagcagaaccgatgagagccgcccttaccgttggtctccggatcccccagtcccatcccgccgttttcggctgtcttcctaaccgtcctgtcttctcttggcgctctttccttccacctttcccaagagtcctggttgcacgttttaagtcatatattttcgtccccctgaaaatgatggcaagcccagtttctcctgagcattcagacccccaggccccagcacttggcgttttcaggaggccctgttcttagagcccctgacaaaggcagcacttatttcctgggctggtgcgccccaaaacacggccccgacacttagtgtggccccaggccccagcgagcctcgccctcccagttttgctctgcccagcagtgttggtgcccagagatgacaagggccagggagcctggcccgggtgtgagaattcagagattctggcctccagctgtcaccacaccgtaacggggccatgtaactgtgcagcatggacagggatgcgacggggcagctggctgtgtccatggccaggtggccagggtcagggctgcaagccaggggtccagggcccttccgttcagcccaaatgctgccccaatgctaactccttggattgtcaacccccatcccccaaatggaaattccgaaggaggcctcctcgcacctgccctccgctgctcctcagaccccagcccccagcgagccgacgtccccacccgttcctgctctcatccccaggttgggcacgtggggttcctcctctgtgggcctggcagacccttcatgagtgggacccaagatatcactgacttcaacccagaggatcgagcccctgcaccctgcctggggccctggggtgtggagcagtggctggggtgggcgtggtgtggcctgagagactgcccagctggagaggccttcctttacaaggccacgcgtgcagctgtcccatccagaccccgactggccaagacctccacgtccccagagtccagccctggaaattccaagggccctggcgtcctctgccttccccgcttccccatgagcgtctgcaaaacacttgcctgaatacatatcacgtattttagactcgaagcctcaaagcactggattgtggtcccctgccccctctgtcccgtccccctgcccaagtgactgaaacctactgagctatattcactgtgctgtcctagggggagggagagcagagctcgcccctgcactgcagccttgtgggggagggcaaggctctcctcccagccagggacgccaggacatagctgctcctggtcagtggaggtcagccgggtatcaaaagccatgaaactgtgtctctgtagcaatgagtgatactgtgacaaaaccatccttgcattcttcctagaagagttcctctgctccttccattccatttttgtgtttgttttgttcttttctgtcactgatccgtattaccacttttggaaaaaaataaataaataaataaataaaaggcagcttgagtttccaaacgtgtgattcacttgtgaacaaaagtcattctaacaattgccttcagcgtcacgtgcattgccactgcgctttcggcacgagggatgctgagccctggtgtcagagtcgtaatttaaagcgtgtgtgtatatggactttgtcccttaaggtcgatataaagaatcctcgcagaatcacagacctgtgccgcccgccaccttctgccattgttacattacagatttggtttagttttgttttgttttgttttttcttttagaactgtatagtattgaaaaagaaatcaaatgtaaatgtctggttttcatataatgtttaaaaagaccattgagaaggaggctggcgctcgccccatgtcccccttgattgtaaattgcttctgttctgtttataagtaaactgtgcatgactcctgcttagcggtcattatcgtgtctgttggtgaaatttttattaaaaggaaaattctgtagatgcacttattgaatatgtgattaggatctacgtctgagactaggagtcctgaactgctgacgcgaaagaggcgcagttcccaattaatacggaaatcgctgtgggagaagaatgaaataagacgtgaagtgtaggaaatcatgaaaagaacaattttgcaaattgcattctgatgcttgtgatgaacacaaatgtacttgtgtagagacatttccttaagagaaagcctaggagaagccgatttggaggttaatgctgtagaataggactgtataccaaatgtaatctttccaatgctccaatgaatttatacatgagattgatatgcaataaatctgtgtgcttttctaaaaaaaaaa

SEQ ID NO: 32 - DNA, 2492 bp, Homo sapiens
gcgccccccgcgcccccgcagcaggaatggcaggaacggatccgggccgagcccgacccgggacgaggaggggctgagccgcgggggatcctggaatcggcagggaggtggggattctgggaggaaaactccatttcctccttccctgccggtttctcccctctaccctggcaccccaaatttcgatgtgccgcctgccgggcgctaagcgccggtgttgagagaggcggcggccgccgcgcccgggaaaatgctgggcatgtacgtgccggacaggttctccctgaagtcctcccgggttcaggacggcatggggctctacacggcccgcagagtgcgaaagggtgaaaagttcggaccctttgctggagagaagagaatgcctgaagacttggatgaaaatatggattacaggttgatgtgggaggttcgtgggagtaagggagaagttttgtacattttggatgctaccaacccacggcactccaactggcttcgcttcgttcatgaggcaccatctcaggagcagaagaacttggctgccattcaagaaggagaaaacattttctatttggcagttgaagatatagaaacagacacggagcttctgattggctacctggatagtgacatggaggctgaggaggaagaacagcaaattatgacagtcatcaaagaaggggaagttgaaaattctagaagacaatcaacagcgggcagaaaagatcgccttggctgtaaagaggactatgcttgtcctcaatgtgaatcgagttttaccagtgaggatattcttgctgagcatctccagacattgcaccagaaacccacagaggagaaagaatttaagtgcaagaactgtgggaagaaattcccagttaagcaggctttgcaaagacatgttcttcagtgcacagcgaaaagcagtctaaaggagtcttcgcgaagttttcagtgctctgtttgcaattcttccttcagttcagcatcgagttttgagcagcaccaggagacttgccggggggatgccaggtttgtgtgcaaggctgacagctgtggaaagaggctgaagagcaaggatgccctgaaaagacaccaggaaaatgtccacactggagatcctaagaaaaagcttatatgttcagtgtgcaataaaaagtgttcttcagcatcaagcctacaggaacatagaaagattcatgagatatttgattgtcaagaatgtatgaagaaatttatttcagctaatcagctaaaacgtcatatgatcacccactcagaaaaacgaccctataattgcgagatttgtaataagtctttcaagaggcttgatcaagtgggtgctcacaaagtaatacacagcgaagacaaaccttacaaatgcaaactttgtggaaagggatttgcccacagaaatgtttacaagaatcataagaagacccactctgaggagagaccgttccaatgtgaagaatgtaaagctttgttccggaccccattttctttacagagacacctgctaatacataacagtgagaggactttcaagtgccatcactgcgatgctacctttaagaggaaggatacattaaatgttcatgtccaggtggttcatgaaagacacaagaagtataggtgtgagctatgtaataaggcctttgttacaccttcagtgcttagaagtcataagaaaacacatacaggagaaaaggagaaaatctgtccatattgtggccagaaatttgccagcagtggtacactcagagttcatatccggagccacacaggtgagcgtccctatcaatgtccttactgtgaaaaaggattcagtaaaaatgatggactgaagatgcacattcgtactcacaccagggagaagccgtacaagtgctcagagtgcagcaaggccttcagccagaagcgaggcctggatgagcacaagaggacgcacactggagaaaagccttttcagtgtgatgtttgtgatttggcttttagcctgaagaaaatgctgattcgacacaagatgactcataatcccaatcgtcccctggcagaatgccagttttgccataagaagtttacaaggaatgactacctcaaagtgcacatggacaatatccatggtgtagctgacagctaataggggctgtaaaggaattaaactattcagaagggctcctaatatgaatcccagatttttatcacctgatcagcataacagaagtagccaaaagtgacttactggtctcatagttcttatttgcctacatttagagtctgtagatgcatatgcaaccaaaaaaaagtcttacttttaacaagaaatggtataaatggaaaaaaaatacaactccatagccttgcaaaatgcaacttgtgctctgtcttataaaaatggagaaaggagtcatggctctctttcttggccatccctctgtaatgaagtttataatatacttccctaataggcctttttaaaaaaaaaaaaa

SEQ ID NO: 33 - DNA, 3142 bp, Homo sapiens
tgcgagctgtttgaggactgggatgccgagaacgcgagcgatccgagcagggtttgtctgggcaccgtcggggtaggatccggaacgcattcggaaggctttttgcaagcatttacttggaaggagaacttgggatctttctgggaaccccccgccccggctggattggccgagcaagcctggaaaatggtaaatgatcatttggatcaattacaggcttttagctggcttgtctgtcataattcatgattcggggctgggaaaaagaccaacagcctacgtgccaaaaaaggggcagagtttgatggagttgggtggacttttctatgccatttgcctccacacctagaggataagcacttttgcagacattcagtgcaagggagatcatgtttgactgtatggatgttctgtcagtgagtcctgggcaaatcctggatttctacactgcgagtccgtcttcctgcatgctccaggagaaagctctcaaagcatgcttcagtggattgacccaaaccgaatggcagcatcggcacactgctcaatcaattgaaacacagagcaccagctctgaggaactcgtcccaagccccccatctccacttcctccccctcgagtgtacaaaccctgcttcgtctgccaggacaaatcatcagggtaccactatggggtcagcgcctgtgagggatgtaagggctttttccgcagaagtattcagaagaatatgatttacacttgtcaccgagataagaactgtgttattaataaagtcaccaggaatcgatgccaatactgtcgactccagaagtgctttgaagtgggaatgtccaaagaatctgtcaggaatgacaggaacaagaaaaagaaggagacttcgaagcaagaatgcacagagagctatgaaatgacagctgagttggacgatctcacagagaagatccgaaaagctcaccaggaaactttcccttcactctgccagctgggtaaatacaccacgaattccagtgctgaccatcgagtccgactggacctgggcctctgggacaaattcagtgaactggccaccaagtgcattattaagatcgtggagtttgctaaacgtctgcctggtttcactggcttgaccatcgcagaccaaattaccctgctgaaggccgcctgcctggacatcctgattcttagaatttgcaccaggtataccccagaacaagacaccatgactttctcagacggccttaccctaaatcgaactcagatgcacaatgctggatttggtcctctgactgaccttgtgttcacctttgccaaccagctcctgcctttggaaatggatgacacagaaacaggccttctcagtgccatctgcttaatctgtggagaccgccaggaccttgaggaaccgacaaaagtagataagctacaagaaccattgctggaagcactaaaaatttatatcagaaaaagacgacccagcaagcctcacatgtttccaaagatcttaatgaaaatcacagatctccgtagcatcagtgctaaaggtgcagagcgtgtaattaccttgaaaatggaaattcctggatcaatgccacctctcattcaagaaatgctggagaattctgaaggacatgaacccttgaccccaagttcaagtgggaacacagcagagcacagtcctagcatctcacccagctcagtggaaaacagtggggtcagtcagtcaccactcgtgcaataagacattttctagctacttcaaacattccccagtaccttcagttccaggatttaaaatgcaagaaaaaacatttttactgctgcttagtttttggactgaaaagatattaaaactcaagaaggaccaagaagttttcatatgtatcaatatatatactcctcactgtgtaacttacctagaaatacaaacttttccaattttaaaaaatcagccatttcatgcaaccagaaactagttaaaagcttctattttcctctttgaacactcaagattgcatggcaaagacccagtcaaaatgatttacccctggttaagtttctgaagactttgtacatacagaagtatggctctgttctttctatactgtatgtttggtgctttccttttgtcttgcatactcaaaataaccatgacaccaaggttatgaaatagactactgtacacgtctacctaggttcaaaaagataactgtcttgctttcatggaatagtcaagacatcaaggtaaggaaacaggactattgacaggactattgtacagtatgacaagataaggctgaagatattctactttagttagtatggaagcttgtctttgctctttctgatgctctcaaactgcatcttttatttcatgttgcccagtaaaagtatacaaattccctgcactagcagaagagaattctgtatcagtgtaactgccagttcagttaatcaaatgtcatttgttcaattgttaatgtcactttaaattaaaagtggtttattacttgtttaatgacataactacacagttagttaaaaaaaatttttttacagtaatgatagcctccaaggcagaaacacttttcagtgttaagtttttgtttacttgttcacaagccattagggaaatttcatgggataattagcaggctggtctaccacctggaccatgtaactctagtgtccttcctgattcatgcctgatattgggatttttttttccagccttcttgatgccaaggggctaattaatattaacaactcccaaagaaacaggcatagaatctgcctcctttgaccttgttcaatcactatgaagcagagtgaaagctgtggtagagtggttaacagatacaagtgtcagtttcttagttctcatttaagcactagtggaattttttttttttgatatattagcaagtctgtgatgtactttcactggctctgtttgtacattgagattgtttgtttaacaatgctttctatgttcatatactgtttacctttttccatggagtctcctggcaaagaataaaatatatttattttaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 34 - DNA, 6853 bp, Homo sapiens
gccaggccgcgtccgccatagtacctggcttggaggtgtcgccgccgctcggtgagagcccccgagcggcagggggccaacacaaaaagggagccggagaagccctagccgctgcccagcagcttgcgggcgtgttctcgcggttccgggcctcaaggcgacggaaacgaaaggcgagcgaagcgcggaggatccggcgagaagaagcgtcagggagcctcggcggtgtccccggggtccgccgaagccacccggccgccggctggggcccggggtggtgaggaagtgctccgaggcctcgccgaggcctagcgccggctttgtgtccgaggcggcggcggcggcggggggaggcggagccgggggcggcctgcgggaaggcctctcctccgccgaccgcgcgttttcggcctaggccgtggggccgctcgtggcctccggggagcaggcgccaggggtttgtgtgcggtgggggcctgggcctgggcctggggaagctgacgccggtcgtccggaagccaggaggaggcgtgaggccgctcgtggactccgggcctaggccctctcccctcaaccttctcccggggcctgggtcaccccaatccacggagagagagacccgccgggaggtgcggccgcgctatggacccctgaccccgtggggtcgctcggactcttaacgtgtggactgaccgctactgactgcaccgccaatccccccgtctctgccggccccttagcatgagcgagggggacccagccgggtgacattgtgcccgttggcggattctcgatttcccctcttccccgtcctcgtcctcctcctcccccatgaagtgattctgagtatcggggggtctctggattattgttctgacgaacccctgcttgtggttggggggtatttaatctgaggccttagggtccttcggtgtctttgagtgttttgtgtgtacatattttgctcttaaagtttataaatatacgtatattgagagtgtccacgtctcctcgctgaaccttaggaatcccttggcaccatgtcctgtgtgcattataaattttcctctaaactcaactatgataccgtcacctttgatgggctccacatctccctctgcgacttaaagaagcagattatggggagagagaagctgaaagctgccgactgcgacctgcagatcaccaatgcgcagacgaaagaagaatatactgatgataatgctctgattcctaagaattcttctgtaattgttagaagaattcctattggaggtgttaaatctacaagcaagacatatgttataagtcgaactgaaccagcgatggcaactacaaaagcaattgatgactcttccgcgtctatttctctggcccagcttacaaagactgccaatctggctgaagccaatgcttctgaagaagataaaattaaagcaatgatgtcgcaatctggccatgaatacgacccaatcaattacatgaagaaacctctaggtccaccacctccatcttacacgtgtttccgttgtggtaaacctggacattatattaagaattgcccaacaaatggggataaaaactttgaatctggtcctaggattaaaaagagcactggaattcccagaagtttcatgatggaagtgaaagatcctaatatgaaaggtgcaatgcttaccaacactggaaaatatgcaataccaactatagatgcagaagcatatgcaattgggaagaaagagaaacctcccttcttaccagaggagccatcttcttcctcagaagaagatgatcctatcccagatgaattgttgtgtctcatctgcaaggatattatgactgatgctgttgtgattccctgctgtggaaacagttactgtgatgaatgtataagaacagcactcctggaatcagatgagcacacatgtccgacgtgtcatcaaaatgatgtttctcctgatgctttaattgccaataaatttttacgacaggctgtaaataacttcaaaaatgaaactggctatacaaaaagactacgaaaacagttacctcctccaccacccccaataccacctccgagaccactgattcagaggaacctacaacctctgatgagatctccgatatcaagacaacaagatcctcttatgattccagtgacatcttcatcaactcacccagctccgtctatatcttcattaacttctaatcagtcttccttggcccctcctgtgtctggaaatccgtcttctgctccagctcctgtacctgatataactgcaacagtatccatatcagttcattcagaaaaatcagatggaccttttcgggattctgataataaaatattgccagctgcagctcttgcatcagagcactcaaagggaacctcctcaattgcaattaccgctcttatggaagagaagggttaccaggtgcctgttcttggaaccccatctttgcttggacagtcattattgcatggacagttgatccccacaactggtccagtaagaataaatactgctcgtccaggtggtggtcgaccaggctgggaacattccaacaaacttggctatctggtttctccaccacaacaaattagaagaggggagaggagctgctacagaagtataaaccgtgggcgacaccacagcgaaagatcacagaggactcaaggcccgtcactaccagcaactccagtctttgtacctgttccaccacctcctttgtatccgcctcctccccatacacttcctctccctccgggtgttcctcctccacagttttctcctcagtttcctcctggccagccaccacccgctgggtatagtgtccctcctccagggtttcctccagctcctgccaatttatcaacaccttgggtatcatcaggagtgcagacagctcattcaaataccatcccaacaacacaagcaccacctttgtccagggaagaattctatagagagcagcgacgactaaaagaagaggaaaagaaaaagtccaagctagatgagtttacaaatgattttgctaaggaattgatggaatacaaaaagattcaaaaggagcgtaggcgctcattttccaggtctaaatctccctatagtggttcttcgtattcaagaagttcatatacttattctaaatcaagatctggttcaacacgttcacgctcttattctcgatcattcagccgctcacattctcgttcctattcacggtcacctccataccccagaagaggcagaggcaagagccgcaattaccgttcacggtctagatctcatggatatcatcgatctaggtcaaggtcacccccttacagacgctatcattcacgatcaagatctcctcaagcgtttaggggacagtctcctaataaacgtaatgtacctcaaggggaaacagaacgtgaatattttaatagatacagagaagttccaccaccatatgacatgaaagcatattatgggagaagtgttgactttagagacccatttgaaaaagaacgctaccgagaatgggagagaaaatatagagagtggtatgaaaaatattataaaggttatgctgctggagcacagcctagaccctcagcaaatagagagaacttttctccagagagatttttgccacttaacatcaggaattctcccttcacaagaggccgcagagaagactatgttggtgggcaaagtcatagaagtcgaaacataggtagcaactatccagaaaagctttcagcaagagatggtcacaatcagaaggataatacaaagtcaaaagagaaggagagtgaaaacgctccaggagatggtaaaggaaataagcataagaaacacagaaaaagaagaaaaggggaggaaagtgagggttttctgaacccagagttattagagacttctaggaaatcaagagaacctacaggtgttgaagaaaataaaacagactcattgtttgttctcccaagtagagatgatgccacacctgttagagatgaaccaatggatgcagaatcaatcacttttaaatcagtgtctgaaaaagacaagagagaaagggataaaccaaaagcaaagggtgataaaaccaaacggaagaatgatggatctgctgtgtccaaaaaagaaaatattgtaaaacctgctaaaggaccccaagaaaaagtagatggagaacgtgagagatctcctcgatctgaacctccaattaaaaaagccaaagaggagactccgaagactgacaatactaaatcatcatcttcctctcagaaggatgaaaaaatcactggaacccccagaaaagctcactctaaatcagcaaaagaacaccaagaaacaaaaccagtcaaagaggaaaaagtgaagaaggactattccaaagatgtcaaatcagaaaagctaacaactaaggaagaaaaggccaagaagcctaatgagaaaaacaaaccacttgataataagggagaaaaaagaaaaagaaaaactgaagaaaaaggcgtagataaagattttgagtcttcttcaatgaaaatctcgaaactagaagtgactgaaatagtgaaaccatcaccaaagcgcaaaatggaacctgatactgaaaaaatggataggacccctgaaaaggacaaaatttctttaagtgcgccagccaaaaaaatcaaactcaacagagaaactgggaagaaaattggaagtacagaaaatatatcaaacacaaaagaaccctctgaaaaattggagtcaacatctagcaaagttaaacaagaaaaagtcaaaggaaaggtcagacgaaaagtgactggaactgaaggatccagctcaactctggtggattacaccagtacgagctcaactggaggcagtcctgtgcggaaatctgaagaaaaaacagatacaaagcgaactgtgattaaaacgatggaagaatataataatgacaataccgcgccagctgaagatgttatcattatgattcaggttcctcaatccaaatgggataaagatgactttgaatctgaagaagaagatgttaaatccacacagcctatatcaagtgtaggaaaacctgctagtgttataaaaaatgttagtacaaagccatcaaatatagtcaagtatcctgagaaagaaagtgagccatccgagaaaattcagaaattcaccaaggacgtgagccatgaaatcatacaacatgaggttaaaagttcaaaaaactctgcatctagtgaaaaagggaaaaccaaagatcgagattattcagtgttggaaaaggagaaccctgaaaagaggaagaacagcactcagccagagaaagagagtaatttggaccgtctgaatgaacaaggaaattttaaaagtctgtctcaatcttccaaagaggctagaacgtcagataaacatgattccactcgtgcttcctcaaataaagacttcactcccaatagagacaaaaaaactgactatgacaccagagagtattcaagttccaaacgtagagatgaaaagaatgaattaacaagacgaaaagactctccttctcggaataaagattctgcatctggacagaaaaataaaccaagggaagagagagatttgcctaaaaaaggaacaggagattccaaaaaaagtaattctagtccctcaagagacagaaaacctcatgatcacaaagccacttatgatactaaacggccaaatgaagagacaaaatctgtagataaaaatccttgtaaggatcgtgagaagcatgtattagaagcaaggaacaataaagagtcaagtggcaataaactactttatatacttaacccaccagagacacaggttgaaaaagagcaaattactgggcaaattgacaagagtactgtcaagcctaaaccccagttaagtcattcctctagactttcctctgacttaactagagaaactgatgaagctgcttttgaaccagactataatgaaagtgacagtgaaagtaatgtttctgtaaaagaagaggaatcttcaggaaacatttctaaggacctgaaagataaaatagtggagaaagcaaaagagagcctggacacagcagcagttgtccaggtgggcataagcaggaatcagagccacagcagccccagcgtcagccccagcagaagccacagtccttctggaagccagacccgaagccacagtagcagtgccagctcagcagaaagtcaggacagcaagaagaagaagaaaaagaaggaaaagaaaaaacacaagaaacataaaaagcataagaagcataagaaacatgcaggcactgaagtggaattggaaaaaagccaaaaacacaaacacaagaaaaagaagtcaaagaagaacaaagataaagagaaggagaaggagaaagatgaccaaaaagtgaaatctgtcactgtgtaaaaagacagattttttaaattgacttaattactaagtcatctgtattaaattttgttataatgtaaagagattcaagccttgtaaataatgacatggaagaccctgtgctgcacttaaaatattgctgcttgattatttgatttttacatcagagctttataacacgaacttttgtacagaattgtgagttgtgaccatgtaacatgagaggttttgctagggcctattatttttaaccaccattaattagttggggtggagtttactgtaatgtgaaattttcacatttgaattttttaattgcctggcaaaagctgatataagttctaaaatatcagcagaatgatttgctgaattcattacaaccctgttatgtcactttttgattacaataaaagttttcagtaaacttttcaaaaaaaaaaaaaaaaaaa

SEQ ID NO: 35 - DNA, 1438 bp, Homo sapiens
ggcagcccacgtccagggagggcggagggaggaggagaggcgcggagaggagggaggggagaggggagggaggaaggcgagagaaagggagctgcttccatcccggacttcccagagcctgcctggagcgcgtactcagcggctctcgggtcccagcgtcccagccgcggcccgcgctcctccgccccgctcctcctcctcctcttcctcctcctcctcctctctaggcacccccgtcccctccttccagcggctgcagcccccagccccaactctccgcgcttactcctgggacgcgcgtcctcgccccatcctttgcttccttccttccttccttcttccttcctcccctggctcccgccctccctctccaggtcgccctcccggggcccgattgtctcggtgccccgctcccggcccgcgccctgccccgtctctcccttgcacttcctgagtcgcccgccgccgccgtcgcagactcgccgcgggagccccagcccaacccgagcccgacagccactgccccggctccagctccagccccacagcccgcggcgcccgcccgagggagccccggcgcccggggaaggctccagtgggctagcgcgccctcgcccagccccgcgccccagccctgcccggcccggcgaggaaggaccgggaagatgaacaacggcggcaaagccgagaaggagaacaccccgagcgaggccaaccttcaggaggaggaggtccggaccctatttgtcagtggccttcctctggatatcaaacctcgggagctctatctgcttttcagaccatttaagggctatgagggttctcttataaagctcacatctaaacagcctgtaggttttgtcagttttgacagtcgctcagaagcagaggctgcaaagaatgctttgaatggcatccgcttcgatcctgaaattccgcaaacactacgactagagtttgctaaggcaaacacgaagatggccaagaacaaactcgtagggactccaaaccccagtactcctctgcccaacactgtacctcagttcattgccagagagccatatgagctcacagtgcctgcactttaccccagtagccctgaagtgtgggccccgtaccctctgtacccagcggagttagcgcctgctctacctcctcctgctttcacctatcccgcttcactgcatgcccagtgtttctctcctgaggcaaagcccaacacacctgtcttttgtccacttctccagcaaattagatttgtctctgggaatgtgtttgtaacataccaacctactgcagaccagcagagggagctcccatgttgaatttgtttgttagctattttcccccctttcacaaaaactatttcttgacgacctttgagagatttcaataaaaattttaatcagagcaaaaaaaaaaaaaaaaaaaaaaaaaa

SEQ ID NO: 36 - DNA, 7274 bp, Homo sapiens
catagagccagcgggcgcgggcgggacgggcgccccgcggccggacccagccagggcaccacgctgcccggccctgcgccgccaggcacttctttccggggctcctagggacgccagaaggaagtcaacctctgctgcttctccttggcctgcgttggaccttcctttttttgttgtttttttttgtttttcccctttcttccttttgaattaactggcttcttggctggatgttttcaacttctttcctggctgcgaacttttccccaattgttttccttttacaacagggggagaaagtgctctgtggtccgaggcgagccgtgaagttgcgtgtgcgtggcagtgtgcgtggcaggatgtgcgtgcgtgtgtaacccgagccgcccgatctgtttcgatctgcgccgcggagccctccctcaaggcccgctccacctgctgcggttacgcggcgctcgtgggtgttcgtgcctcggagcagctaaccggcgggtgctgggcgacggtggaggagtatcgtctcgctgctgcccgagtcagggctgagtcacccagctgatgtagacagtggctgccttccgaagagtgcgtgtttgcatgtgtgtgactctgcggctgctcaactcccaacaaaccagaggaccagccacaaacttaaccaacatccccaaacccgagttcacagatgtgggagagctgtagaaccctgagtgtcatcgactgggccttcttatgattgttgttttaagattagctgaagatctctgaaacgctgaattttctgcactgagcgttttgacagaattcattgagagaacagagaacatgacaagtacttctagctcagcactgctccaactactgaagctgattttcaaggctacttaaaaaaatctgcagcgtacattaatggatttctgttgtgtttaaattctccacagattgtattgtaaatattttatgaagtagagcatatgtatatatttatatatacgtgcacatacattagtagcactacctttggaagtctcagctcttgcttttcgggactgaagccagttttgcatgataaaagtggccttgttacgggagataattgtgttctgttgggactttagacaaaactcacctgcaaaaaactgacaggcattaactactggaacttccaaataatgtgtttgctgatcgttttactcttcgcataaatattttaggaagtgtatgagaattttgccttcaggaacttttctaacagccaaagacagaacttaacctctgcaagcaagattcgtggaagatagtctccactttttaatgcactaagcaatcggttgctaggagcccatcctgggtcagaggccgatccgcagaaccagaacgttttcccctcctggactgttagtaacttagtctccctcctcccctaaccacccccgcccccccccaccccccgcagtaataaaggcccctgaacgtgtatgttggtctcccgggagctgcttgctgaagatccgcgcccctgtcgccgtctggtaggagctgtttgcagggtcctaactcaatcggcttgttgtgatgcgtatccccgtagatgccagcacgagccgccgcttcacgccgccttccaccgcgctgagcccaggcaagatgagcgaggcgttgccgctgggcgccccggacgccggcgctgccctggccggcaagctgaggagcggcgaccgcagcatggtggaggtgctggccgaccacccgggcgagctggtgcgcaccgacagccccaacttcctctgctccgtgctgcctacgcactggcgctgcaacaagaccctgcccatcgctttcaaggtggtggccctaggggatgttccagatggcactctggtcactgtgatggctggcaatgatgaaaactactcggctgagctgagaaatgctaccgcagccatgaagaaccaggttgcaagatttaatgacctcaggtttgtcggtcgaagtggaagagggaaaagcttcactctgaccatcactgtcttcacaaacccaccgcaagtcgccacctaccacagagccatcaaaatcacagtggatgggccccgagaacctcgaagacatcggcagaaactagatgatcagaccaagcccgggagcttgtccttttccgagcggctcagtgaactggagcagctgcggcgcacagccatgagggtcagcccacaccacccagcccccacgcccaaccctcgtgcctccctgaaccactccactgcctttaaccctcagcctcagagtcagatgcaggatacaaggcagatccaaccatccccaccgtggtcctacgatcagtcctaccaatacctgggatccattgcctctccttctgtgcacccagcaacgcccatttcacctggacgtgccagcggcatgacaaccctctctgcagaactttccagtcgactctcaacggcacccgacctgacagcgttcagcgacccgcgccagttccccgcgctgccctccatctccgacccccgcatgcactatccaggcgccttcacctactccccgacgccggtcacctcgggcatcggcatcggcatgtcggccatgggctcggccacgcgctaccacacctacctgccgccgccctaccccggctcgtcgcaagcgcagggaggcccgttccaagccagctcgccctcctaccacctgtactacggcgcctcggccggctcctaccagttctccatggtgggcggcgagcgctcgccgccgcgcatcctgccgccctgcaccaacgcctccaccggctccgcgctgctcaaccccagcctcccgaaccagagcgacgtggtggaggccgagggcagccacagcaactcccccaccaacatggcgccctccgcgcgcctggaggaggccgtgtggaggccctactgaggcgccaggcctggcccggctgggccccgcgggccgccgccttcgcctccgggcgcgcgggcctcctgttcgcgacaagcccgccgggatcccgggccctgggcccggccaccgtcctggggccgagggcgcccgacggccaggatctcgctgtaggtcaggcccgcgcagcctcctgcgcccagaagcccacgccgccgccgtctgctggcgccccggccctcgcggaggtgtccgaggcgacgcacctcgagggtgtccgccggccccagcacccaggggacgcgctggaaagcaaacaggaagattcccggagggaaactgtgaatgcttctgatttagcaatgctgtgaataaaaagaaagattttatacccttgacttaactttttaaccaagttgtttattccaaagagtgtggaattttggttggggtggggggagaggagggatgcaactcgccctgtttggcatctaattcttatttttaatttttccgcaccttatcaattgcaaaatgcgtatttgcatttgggtggtttttatttttatatacgtttatataaatatatataaattgagcttgcttctttcttgctttgaccatggaaagaaatatgattcccttttctttaagttttatttaacttttcttttggacttttgggtagttgtttttttttgttttgttttgtttttttgagaaacagctacagctttgggtcatttttaactactgtattcccacaaggaatccccagatatttatgtatcttgatgttcagacatttatgtgttgataattttttaattatttaaatgtacttatattaagaaaaatatcaagtactacattttcttttgttcttgatagtagccaaagttaaatgtatcacattgaagaaggctagaaaaaaagaatgagtaatgtgatcgcttggttatccagaagtattgtttacattaaactccctttcatgttaatcaaacaagtgagtagctcacgcagcaacgtttttaataggatttttagacactgagggtcactccaaggatcagaagtatggaattttctgccaggctcaacaagggtctcatatctaacttcctccttaaaacagagaaggtcaatctagttccagagggttgaggcaggtgccaataattacatctttggagaggatttgatttctgcccagggatttgctcaccccaaggtcatctgataatttcacagatgctgtgtaacagaacacagccaaagtaaactgtgtaggggagccacatttacataggaaccaaatcaatgaatttaggggttacgattatagcaatttaagggcccaccagaagcaggcctcgaggagtcaatttgcctctgtgtgcctcagtggagacaagtgggaaaacatggtcccacctgtgcgagaccccctgtcctgtgctgctcactcaacaacatctttgtgttgctttcaccaggctgagaccctaccctatggggtatatgggcttttacctgtgcaccagtgtgacaggaaagattcatgtcactactgtccgtggctacaattcaaaggtatccaatgtcgctgtaaattttatggcactatttttattggaggatttggtcagaatgcagttgttgtacaactcataaatactaactgctgattttgacacatgtgtgctccaaatgatctggtggttatttaacgtacctcttaaaattcgttgaaacgatttcaggtcaactctgaagagtatttgaaagcaggacttcagaacagtgtttgatttttattttataaatttaagcattcaaattaggcaaatctttggctgcaggcagcaaaaacagctggacttatttaaaacaacttgtttttgagttttcttatatatatattgattatttgttttacacacatgcagtagcactttggtaagagttaaagagtaaagcagcttatgttgtcaggtcgttcttatctagagaagagctatagcagatctcggacaaactcagaatatattcactttcatttttgacaggattccctccacaactcagtttcatatattattccgtattacatttttgcagctaaattaccataaaatgtcagcaaatgtaaaaatttaatttctgaaaagcaccattagcccatttcccccaaattaaacgtaaatgttttttttcagcacatgttaccatgtctgacctgcaaaaatgctggagaaaaatgaaggaaaaaattatgtttttcagtttaattctgttaactgaagatattccaactcaaaaccagcctcatgctctgattagataatcttttacattgaacctttactctcaaagccatgtgtggagggggcttgtcactattgtaggctcactggattggtcatttagagtttcacagactcttaccagcatatatagtatttaattgtttcaaaaaaaatcaaactgtagttgttttggcgataggtctcacgcaacacatttttgtatgtgtgtgtgtgtgcgtgtgtgtgtgtgtgtgtgaaaaattgcattcattgacttcaggtagattaaggtatctttttattcattgccctcaggaaagttaaggtatcaatgagacccttaagccaatcatgtaataactgcatgtgtctggtccaggagaagtattgaataagccatttctactgcttactcatgtccctatttatgatttcaacatggatacatatttcagttctttctttttctcactatctgaaaatacatttccctccctctcttccccccaatatctccctttttttctctcttcctctatcttccaaaccccactttctccctcctccttttcctgtgttctcttaagcagatagcacatacccccacccagtaccaaatttcagaacacaagaaggtccagttcttcccccttcacataaaggaacatggtttgtcagcctttctcctgtttatgggtttcttccagcagaacagagacattgccaaccatattggatctgcttgctgtccaaaccagcaaactttcctgggcaaatcacaatcagtgagtaaatagacagcctttctgctgccttgggtttctgtgcagataaacagaaatgctctgattagaaaggaaatgaatggttccactcaaatgtcctgcaatttaggattgcagatttctgccttgaaatacctgtttctttgggacattccgtcctgatgatttttatttttgttggtttttatttttggggggaatgacatgtttgggtcttttatacatgaaaatttgtttgacaataatctcacaaaacatattttacatctgaacaaaatgcctttttgtttaccgtagcgtatacatttgttttgggatttttgtgtgtttgttgggaattttgtttttagccaggtcagtattgatgaggctgatcatttggctctttttttccttccagaagagttgcatcaacaaagttaattgtatttatgtatgtaaatagattttaagcttcattataaaatattgttaatgcctataactttttttcaatttttttgtgtgtgtttctaaggactttttcttaggtttgctaaatactgtagggaaaaaaatgcttctttctactttgtttattttagactttaaaatgagctacttcttattcacttttgtaaacagctaatagcatggttccaattttttttaagttcactttttttgttctaggggaaatgaatgtgcaaaaaaagaaaaagaactgttggttatttgtgttattctggatgtataaaaatcaatggaaaaaaataaactttcaaattgaaatgacggtataacacatctactgaaaaagcaacgggaaatgtggtcctatttaagccagcccccacctagggtctatttgtgtggcagttattgggtttggtcacaaaacatcctgaaaattcgtgcgtgggcttctttctccctggtacaaacgtatggaatgcttcttaaaggggaactgtcaagctggtgtcttcagccagatgacatgagagaatatcccagaaccctctctccaaggtgtttctagatagcacaggagagcaggcactgcactgtccacagtccacggtacacagtcgggtgggccgcctcccctctcctgggagcattcgtcgtgcccagcctgagcagggcagctggactgctgctgttcaggagccaccagagccttcctctctttgtaccacagtttcttctgtaaatccagtgttacaatcagtgtgaatggcaaataaacagtttgacaagtacatacaccata

SEQ ID NO: 37 - DNA, 7769 bp, Homo sapiens
ccctcctgcctgtccctttctgtcttctctctctctctcccacttttcctccctctcccgctccgtctcacacgcaccctctgtttattttcctgcctccatctgggccctgctgatattgtaatcaccctgatgcacgttggcttctctcctctccctcctgcgctcacacactcactcacacacaatgtgccatcctgacaaggcttttacttctgataagctccaatgtgtgtttaatgaatacaaagccgcggtctgggtgccgcctcggccgcggccgctctcccgcgctcctttgccagaagctggcggagagaatttacatttaaagattagcagagtgagaaagagaaatctgccttttgttgtgtggggtgaggaggaggcatctacccctggccttgacgctatctcccatcacctctgctatccagacaggactcaccgagtatcaaatgataagaactgagcacactggatgaaataatacttgcactgggagcactaagaaaagattgatctctgggctggtgaacataatctctgtcccagtcagaaaaggagagaggaaattagcagagcgattggtggagaatgatatctgtcaaaagaaacacttggagagcactgagtttagtaataggtgactgccggaaaaaagggaactttgaatattgtcaagaacaggaggcatgagcccggaacgcgcttgcttttaggagacagccactttctgtgtggtacgctggattcaaggatgcctgatcgtactgagaagcactccacaatgccagactcacctgtggatgtgaagacgcaatctaggctgactcctccaacaatgccacctcccccaactactcaaggagctccaagaaccagttcatttacaccgacaacgttaactaatggcacgagccattctcctacagccttgaatggcgccccctcaccacccaatggcttcagcaatgggccttcctcttcttcctcctcctctctggctaatcaacagctgcccccagcctgtggtgccaggcaactcagcaagctgaaaaggttccttactaccctgcagcagtttggcaatgacatttcacccgagataggagaaagagttcgcaccctcgttctgggactagtgaactccactttgacaattgaagaatttcattccaaactgcaagaagctactaacttcccactgagaccttttgtcatcccatttttgaaggccaacttgcccctgctgcagcgtgagctcctccactgcgcaagactggccaaacagaaccctgcccagtacctcgcccagcatgaacagctgcttctggatgccagcaccacctcacctgttgactcctcagagctgcttctcgatgtgaacgaaaacgggaagaggcgaactccagacagaaccaaagaaaatggctttgacagagagcctttgcactcagaacatccaagcaagcgaccatgcactattagcccaggccagcggtacagtccaaataacggcttatcctaccagcccaatggcctgcctcaccctaccccacctccacctcagcattaccgtttggatgatatggccattgcccaccactacagggactcctatcgacaccccagccacagggacctcagggacagaaacagacctatggggttgcatggcacacgtcaagaagaaatgattgatcacagactaacagacagagaatgggcagaagagtggaaacatcttgaccatctgttaaactgcataatggacatggtagaaaaaacaaggcgatctctcaccgtactaaggcggtgtcaagaagcagaccgggaagaattgaattactggatccggcggtacagtgacgccgaggacttaaaaaaaggtggcggcagtagcagcagccactctaggcagcagagtcccgtcaacccagacccagttgcactagacgcgcatcgggaattccttcacaggcctgcgtctggatacgtgccagaggagatctggaagaaagctgaggaggccgtcaatgaggtgaagcgccaggcgatgacggagctgcagaaggccgtgtctgaggcggagcggaaagcccacgacatgatcacaacagagagggccaagatggagcgcacggtcgccgaggccaaacggcaggcggcggaggacgcactggcagttatcaatcagcaggaggattcaagcgagagttgctggaattgtggccgtaaagcgagtgaaacctgcagtggctgtaacacagcccgatactgtggctcattttgccagcacaaagactgggagaagcaccatcacatctgtggacagaccctgcaggcccagcagcagggagacacacctgcagtcagctcctctgtcacgcccaacagcggggctgggagcccgatggacacaccaccagcagccactccgaggtcaaccaccccgggaaccccttccaccatagagacaacccctcgctagacgtgaactcagaactgtcggaggaaagacaacacaaccaacgcgaaaccaattcctcatcctcagatgctcaaagttgttttttttgtttgtttgtttattagatgaattatcctatttcagtacttcagcaagagagaacctaactgtatcttgaggtggtagtaaaacacagagggccagtaacgggtcgtaatgacttattgtggataacaaagatatcttttctttagagaactgaaaagagagcagagaatataacatgaaatgatagatttgacctcctccctgttattttcaagtagctgggattttaaactagatgacctcattaaccgatgctttaccaaacagcaaaccaagagattgctaattgctgttgaaagcaaaaatgctaatattaaaagtcacaatgttctttatatacaataatggaaaaaaaaaaaagaggaaaaccctcaagggcatgagcattggatacagcagtagacattttaacaagaagatgaatggcgtccgtgggttgctaactgaactttgaagacccgctacaaaacgcgcagatgtgcagcagattggaaggagacacagatgttcggtttttttcctgtttctgaaaagaaataataatatccaggtcaacagaatgaaaaatgaaagatgatttgcaatgggatgtatgaatacagcagcaagaaaaaaaaatgccataatacaaaaggctccatatatatatatatatatatatatatacacacacacacacgcacatatacacacacacacacacacacacacacacacacacacacacacacacacacacacaagtaagagactcagcctgcagttaattagcattctggcagttttgacatcagccagctgccctaaataacccttcaacgtttcttcacttttgcaaggttccacagagtaagacattgggtctattccagctcattcattttatattgaaaaaaataattttaaaaatggtggcttcagctccagcccctttccaaaatttttcaaccccaccctgtttggatttttaattaaaaactagtagttctcttggtgttaaaacacttctgtcctgtgaggtttcccaatggtgtttttcttgtaaatgtgttggacaaatgtgaagatgcattgtagtttaaccatatgcccacatttagtctctttattcctagttggtgagaaacctgtatctttctatgctgcttttatatctgtatgtattagtgatatttctctagtagttaaaaaaaaaaaaaggaaaaaaagactctttttggttgtcctcaagaaaagaaaagaagaaaagctatctttcggagctgctatttcactctcttataggatttttttttttcctgaaaaccagcatgcttcacggaatgctaacatattggtgtttttgtaagagggatgtacataaatgtattttgcactgtcacaatgactccctaggcatgctttttgtttttttttttttttttttgagcaaattctttttaaatatgctagagccatttcatcaagtttagctgtagcatagagtagtcgtggaatttttcttttttttttttttaaatcattattagggacttttttaaaaaaataaaataacaagatatcctactttaaaaaaaaaggacagtgcatgcatattgctgtaaggtcgtttaagtatttctaattttacaaaagtaaaaatcattagagctccaaatgctgaggtgtagattgacagctttaacactgctgaatgccaggttatacagtattcttacaaaaagggaagtcagccaaagactgatctgatggaccaaaatcggaattttgaaaagcaccagtactactttccaaatgatcagcaggttaaacatgttcagcaaggtatagtatgaatccattaacccttcgttgtccagggtccagaccagaactacttgttagttttgaaaacagtagcccaggatgtggttgtgtgctttaggaaagtcagcagtgctgcaggctgtgtagacaaccaaagtttacaaggctgggaagacacttctccaaatacatatacaccaggcatttctttaaaatcaaaaacaatccattagctttggggcggggggtggggggagggttaaatgtaggccttggaaacagttttaagtggcttttaagtatctgcatagaggcaatcctaaggtttgctttaagtctcttaaatctaacatttttaaaagcatttcttccctgtattttcatatacaagttggttttaattttgtttgggtggtgttattttttttaaatgactttttagtttagaaagttctgatgagcaaataaatacatctatctacatgttggaagtccatctgccagcacacctgcttaaagtgaaatgaaagcacataaccaggctctgaatgggttatattttatcatggtgctcccagaatcctttgacctcactctgctttctagtctgcttgtgacactggacagtcctttcttacagagctattatatgttctaggctacgaagggttaaataatcccaaaaatgaggtatgtcatacccacacgtctttatctctagaagctgtgatgtatgtgaaacagcactgttattcttaacactagtgtgtaaaataaggattagtacagtacgtctcattactttttaattcagggcaccctgagtgaaaacaaatacaaaaaaaaatccctaactctgagctctatctctgattcctcttcctccttcccctcttcctcctcttcctctcctcctgttttgctacattctcctcagtggcaaaaagtttcactctacctctgacagcatgtatattgcaccagtagctaacaaaaactggtctagtcaaaccaaatgggcacaaaagaaccaggataccaaaagttaagctcatacagctgcaaaccatatcacttcttggtaacaatgcagacctcataaacctaaagaagagaaagaaaagaaaacttttgttactttccttttttgcttgtcacttatatacaggctatgtgagaatataatttgtaggtataacacattaagaaaaagttatcttcattggatagaattgaatggtggtcgctgataggaatagggcgtcctctagctcttatctctgtctcttactcttttctctttctctttttctctgtcatgagactgtgtgtgacagggccacctgtcttttttttttcttaaattttttttttttttatgtgtaggtgcatgtcttggggatttaaaaatttcaaggctggtttacttatgcaaagcatgcctacgtctggaatacttagggaaagaaagcgactccatgttgtccgaattcctcaagggacagaaaaaaaattggagactgttgaaatgcagatttgaagtaatttttttaaaatattattttgggttctgcgacattgtgaaaaattaaagttgttgtgcaatacttaattcagacatgtaccacaagttaatggtagactaacactggggggtggggtctaggcatcatgcttttgtcagcatactcttgagcttttaagtctactatgtctgaactgtggtttcttgtttatccttttttccttagttggactgtaatgtatggtctgtcaacctgtgaatctttaaagtatgattcaggtattgttgtattctttactgtgtaataaaaaagttgaaaaaaatctggatcctctgtctccctcgtccccggtgtgccatggttgcccaccagtgaccccagcctgaggagtccatcctccatggtgctgaatcaaatctgtagagtttattcagctacaaagcgtggctctgtaacaggagttggcttatccatgcgtgttgagtagggaatgatgtcagaactttgcaaaagtcgagctcattcataaggggatgtttctcaggcaggaggagtaggaagagtgggaggagaattactaccttccataggaaaggagagcttggcttgcctagattagctgctgcatagatagcagaggcccttttggctgtgttttgataaaattagaaatgagtacctaaatctggatcactctgctcagagaggttcgtgccaacctcatgaggctctgactcttgtcaggttgcttggctgccttgtgcatacccgaatggcagatcagctgacatgacatggaactgctctatctgccttgtcccctctcctctaattaaaagtcgctaccattgtcactcttggttatgtagttaatatccactgtggctatctccaacttgctgggacatccagttatctcctctgacacatattttttcattctctagttacaaagtttgttagagtttgaaaaacaaacccttctcccctaccctgtgaatgctgttagtttttatgtgtagttttcaaaacacagattatcttatatactgtgttgtagcagaaattcctgtttcctctgaatgttctgactttatagaccaaaataatcgttgcaaaaaatcaaggatggcacagcaggtgcatgagcccgtgtctggagaaaggttatgaaggtgaatgctggttacatgggagggtgacttggagccctctagtttccttcttgtgccagctgggttcctgtttcaaggccaggcttctctaacttgtcttcctgggaaatgtagcttttccaagaatttggtagtgcacaccaggaaaaactaggtatcagaacagatttttttttaaatagtggaaacaccctcactcccacactttggacagttagggttggatatttaatttcatagttactctttttttccacagaagaaaggccagcctgacccaagttgcctatttttaatgtgttactgcatcttattttctcttcaaagtgaaaaaggccagaaactagcagatagaaatggaacccgttgacttagttcccttataagaagttcttgcaaacacctctgtgacgttttcattcaagtggatatgttggacaaataggaaacagttttagtttacgcgaacagtatgagacttccaggtggtggacgccatttttttagaagtgttgatgatgtatttgataacattataaaagtatgtgtctcccgtgaaagaacattgagctatttccctggaatggatggggaagaaatgagcaaaggagtgctgttctgtatgggtgtctatacccttattccaaggtgaataaataaagttgtgtttgcctaatcatg

SEQ ID NO: 38 - DNA, 1293 bp, Homo sapiens
aaaagaacgaagtccagcaccaaaacgtgctacaacatggatgaacttcgatgactttgtgccacatgaaagaagaagccagccacaaaaggccatatattgtatgaaatgaaatgtccagaatgggcaaacccatagagacacaaaaatctccgccacctccctactctcggctgtctcctcgcgacgagtacaagccactggatctgtccgattccacattgtcttacactgaaacggaggctaccaactccctcatcactgctccgggtgaattctcagacgccagcatgtctccggacgccaccaagccgagccactggtgcagcgtggcgtactgggagcaccggacgcgcgtgggccgcctctatgcggtgtacgaccaggccgtcagcatcttctacgacctacctcagggcagcggcttctgcctgggccagctcaacctggagcagcgcagcgagtcggtgcggcgaacgcgcagcaagatcggcttcggcatcctgctcagcaaggagcccgacggcgtgtgggcctacaaccgcggcgagcaccccatcttcgtcaactccccgacgctggacgcgcccggcggccgcgccctggtcgtgcgcaaggtgccccccggctactccatcaaggtgttcgacttcgagcgctcgggcctgcagcacgcgcccgagcccgacgccgccgacggcccctacgaccccaacagcgtccgcatcagcttcgccaagggctgggggccctgctactcccggcagttcatcacctcctgcccctgctggctggagatcctcctcaacaaccccagatagtggcggccccggcgggaggggcgggtgggaggccgcggccaccgccacctgccggcctcgagaggggccgatgcccagagacacagcccccacggacaaaaccccccagatatcatctacctagatttaatataaagttttatatattatatggaaatatatattatacttgtaattatggagtcatttttacaatgtaattatttatgtatggtgcaatgtgtgtatatggacaaaacaagaaagacgcactttggcttataattctttcaatacagatatattttctttctcttcctccttcctcttccttactttttatatatatatataaagaaaatgatacagcagagctaggtggaaaagcctgggtttggtgtatggtttttgagatattaatgcccagacaaaaagctaataccagtcactcgataataaagtattcgcattatagttttttttaaaaaaaaaa

SEQ ID NO: 39 - DNA, 5018 bp, Homo sapiens
gcccgggactatcccttcgcggtgtagcggcagccggagacctggctgaggaggcaaccgcgtagacacctccctgcttagaaaacaaacactgaaccagaccgatcccagttggagggttcgaaaatgttccagacagcctgtcgggaggggttgttgttgctgttggactaaatagctattcctgattggtcatgtatagggttttttaaggcgggtggggggaggagggggtagaggaaaggctccaaacacctgcaggttgggggcggaaagctgtttgcgattccctggactggttggtcggggacaggaggtaattcccagccattgacccccatttctctctctccctccctcttgccctgcctctttctctccacccctatctttcctggaaactcgctttgggcgcggcagatcgcccaggaccacaccgcagcgtaactgcaggcctctcagcgaaaaagggggaaagcaaagacccgggtgtgcatcctcttcctcggcttccgcccctttccggcggagtggagatcctattcagaggggccggtctctctaaatatgccccaggatgaccgagcggccgccgagcgaggcggctcgcagtgacccccagctagagggacgggacgcggccgaggccagcatggcccccccgcacctggtcctgctgaacggcgtcgccaaggagacgagccgcgcggccgcagcggagcccccagtcatcgaactgggcgcgcgcggaggcccggggggcggccctgccggtgggggcggcgccgcgagagacttaaagggccgcgacgcggcgacggccgaagcgcgccatcgggtgcccaccaccgagctgtgcagacctcccgggcccgccccggcccccgcgcccgcctcggttacagcggagctgcccggcgacggccgcatggtgcagctgagtcctcccgcgctggctgcccccgccgcccccggccgcgcgctgctctacagcctcagccagccgctggcctctctcggcagcgggttctttggggagccggatgccttccctatgttcaccaccaacaatcgagtgaagaggagaccttccccctatgagatggagattactgatggtccccacaccaaagttgtgcggcgtatcttcaccaacagccgggagcgatggcggcagcagaatgtgaacggggcctttgccgagctccgcaagctgatccccacacatcccccggacaagaagctcagcaagaatgagatcctccgcctggccatgaagtatatcaacttcttggccaagctgctcaatgaccaggaggaggagggcacccagcgggccaagactggcaaggaccctgtggtgggggctggtgggggtggaggtgggggagggggcggcgcgcccccagatgacctcctgcaagacgtgctttcccccaactccagctgcggcagctccctggatggggcagccagcccggacagctacacggaggagcccgcgcccaaacacacggcccgcagcctccatcctgccatgctgcctgccgccgatggagccggccctcggtgatgggtctgggccaccaggatcagccaggagggcgttcttaggctgctgggatggtgggcttcagggcaggtggggtgagaattgggcggctctgaagcaaggcggtggacttgaactttcctggatgtctgaactttgggaagcctttactgaccctggggctggcttttctgtttcctgtaccagtaggagatcagaaaaatggagcaaagtggtaggtactttttgtgaagacggcacggtcttccctcttccctcagtcccaaatccttcccaagtaagaggctggagttgtcactgcttttggcctggagtttgggatccctgtctttcctaagacctggggttgtcagctctcatctgaggcatccagcagtctctgccttgcctttagcccctcccaagctggctggggtggcctgtgtggccacttctgtccatatttataggtacccaatagctgcccatttcgtgagccccatcttcacccaggcctatgttgatccatccagcttgccagatgctgcagagtcacaagcctcgaggtgccttcttcagggcctggttgaagaagatgatcagtggacagtctgctctagatgagctgggccggagggtcaggaaacccagtcgcccttacttcttgccctggggatcaaagttctgctttctccccaatgagacttgccttcctaagcctgtggctgtggagacaatgtctgcagccctgagaaagccctgtcgggctttgtgtgaaggcagagaaagggacaatgatagtagagtgatatggagcaagagatattttgggcatgtgggcttcaactcctcgacatcactgttcatgctggcgagtgaatgccagtgtgctgatgggcgtacgctggtgctgagtagatgcgcagccccatctgtgcattctcctggatgcttagagggatttctttgctgtaagatgtctgtttgctgatggtctggtctatgttccgaattgagcacaaaacctgtcctatgaatgctttgcatttggaatttttgcttgacttcagttattggtggaatctttagcgctcaataggaccaggatccagcctcacttctagggtatgggaaatccaatcagagaccaggccctggctaagacccaaacatatgcacattcacttagcagaaccttaaacacccctcagttgtgcagcttttggtcatcaagggtgcgtctgggaggttggtttaatgcaatagaagtgctcccctctgaaagttgtacatgaaatttttgtaaatcacatccttatccttcatcttttaaagaaataaccactgcaagtccttttgtaaagtgaagaatccttttgtagaatgaaccactgccccttcattgatttcctgtgtcaatccagatggtgggatgtggttttcttaaggtgaggcctgtctgtgacctgcatctaagcccatgggacaaattgcacagaagtcctgtatgtctgtcattgtacccttaagtcaccctagccctctccctctaggctctgccttcgaggtcagaggagagatagcctgtggccctgtcctgccatgcaagaactcatcactgtggctgtctggaaagcccccccttatagtttgggcttcagcctagtggcttgtcctcaccatgatggggccctaattcagccatgtacagacagagaatatgtctgctcctttccccttccttttaagtaaggtccaattctcgagcttggggcaacattgttcacctttgtagcactcaggctctccattcaatttcaggctccccagatcatgttttggtgaaaattagggttggttcctttccaacgtttggaagatcctgtgaggagccccatctgtctaaagatagagtcattgctgtaggatctaaggctgtttgcttcaccgtggattcgcttgagttaggaatgagaagtagccacagtatggatgggtggatgggttttatgagatggatcacatattttattaagaactcaaacttctggctccctcttctttcagacttgccatgtgactctggcttggcctatctcctagggctatggtgtggactgaatgggatcatgaaagtagacagttttgagaacgtaaagaactttttcttttccctcaatctcaatcctgcagtggggtttcgcagcctgagtccacgacctaggcagtaggccggtgtgcctgactgcccagcatttgggtaatttagattgtaaaccgctttggcctgagttattgagattgtcctcatttctccagattatctatttgtgtgtgtgtgtgtgtgtgtgtgagagacggtgtcttgttctgtcactcaggctggagtacagtggtgccatcattgctgtctgcagccttgaactctgggctcaagcaatcctctcacctcagcctcccgagtagggaggaccacaggtgtgagccaccacacctggctaatttttacttttttttttttttggtagagatggagtcttgctatattgcccaggctggtcttgaagtcctggcttcaggcaattctcctgcctttgcctccagaagcactgggatcacaggtgtcagccattgcacccagcccagattgtcttaatttctatcttgttccaaggccagggacagtaataagaatggaaaagagatatgggaacactggcagactgtgtaaaatgtaatgcaactacccaaaacaagcctggtaggaaagggcaagtctttaggtctttgtaagaactaaagaagatctgtaatttttattttcaccctctgtaccccatgaccttatccttcctctccttccttgttacccatgaaaaactggcaacattccaagaatagcatctgtacaaaggggaaagaacataaaggtaaaacaaaacaaaacaacattttgagaacaaagatgaccataaccactgaagggaatcacatcttttaagacaaattcatattcttttatttgttatggcagatgacaagatggtacaacctttattcttttccaaaataaaacaaagggcacagcatctgtagtcagccgacaactatttcggccttttgggggtgggtctggccgtacttgtgatttcgatggtacgtgaccctctgctgaagacttgccccctgcccgtgtacatagtgcattgtttctgtgggcgggcccagcactttccgtcaacgttgtactgtatgtgatgaattgcgttggtctctgcatttttctgcagaagaggagtaaccgctccaggtaccttgacctttgtacagcccagaggccaacactgtgggtgtgtgactctttagcaaaaaaaacccatgtggtgatgatgtgtatatatatgtgaggatgtatcgggaagatttctaaataaaagttttacaaaggggaaaaaaaaaaaaaaaa

SEQ ID NO: 40 - DNA, 1227 bp, Homo sapiens
cggcgcacggagggacgcggccggcgcccatggcgttcgcgctgctgcggcccgtcggcgcgcacgtgctgtacccggacgtgcggctgctgagcgaggacgaggagaaccgcagcgagagcgacgcgtcggaccagtcgttcggctgctgcgagggcccggaggcggcgcggcgcggcccgggccccgggggcgggcggcgggcgggcggcggcggcggcgcgggccccgtggtggtggtgcgacagcggcaggcggccaacgcgcgggagcgggaccgcactcagagcgtgaacacggccttcacggcgctgcgcacgctcatccccaccgagccggtggaccgcaagctgtccaagatcgagacgctgcgcctggcgtccagctacatcgcgcacctggccaacgtgctgctgctgggcgactcggccgacgacgggcagccgtgcttccgtgccgcgggcagtgccaagggcgccgtccccgccgccgccgacggcggccgccagccgcgctccatctgcaccttctgcctcagcaaccagcgcaaggggggtggccgtcgtgacctggggggcagctgcttgaaggtgaggggggtggccccccttcgagggccacggagatgagcctggaccctggagaaggaggccaggagccagccactggctggacagggaagaagaccccaggagccaagcccaccccttctttgtgtagggaccgggggaccatggcctgttccgggacactctgggcagggccctcgggacatctccacccgatcctggagagctgtgaggatccattcagcctgcccagctctggctggtcagagacaaggcagaacttttggaaaaacaaagactgttggtgacagggtgtgtgtgtatctgtgcgtgagtgtgagtgtgtgtgagagagaattggtgagtttaaaataaaagctatttttaaataaaagacgtcgttctgagctgaggagagtcccgtggacatcggaggtcagaggtgggccctcccctggacctagagagaggcacagatgttctgggggctgccctgagccctggaagggtgggggtggggcgggggaaggcaggcagagccccctctccgggcgccataaccaaactttcctcagtctgctgtttatcagatgttcttaacaactgttaggtgtctgatatggttggtaaagaaatcttaattttacttatttttgaa

SEQ ID NO: 41 - DNA, 4669 bp, Homo sapiens
ctgcttgtcaaaaggcggcagcggagccgtgtgcgccgggagcgcggaacagcttgtccacccgccggccggaccagaagcctttgggtctgaagtgtctgtgagacctcacagaagagcacccctgggctccacttacctgccccctgctccttcagggatggaggcaatggcggccagcacttccctgcctgaccctggagactttgaccggaacgtgccccggatctgtggggtgtgtggagaccgagccactggctttcacttcaatgctatgacctgtgaaggctgcaaaggcttcttcaggcgaagcatgaagcggaaggcactattcacctgccccttcaacggggactgccgcatcaccaaggacaaccgacgccactgccaggcctgccggctcaaacgctgtgtggacatcggcatgatgaaggagttcattctgacagatgaggaagtgcagaggaagcgggagatgatcctgaagcggaaggaggaggaggccttgaaggacagtctgcggcccaagctgtctgaggagcagcagcgcatcattgccatactgctggacgcccaccataagacctacgaccccacctactccgacttctgccagttccggcctccagttcgtgtgaatgatggtggagggagccatccttccaggcccaactccagacacactcccagcttctctggggactcctcctcctcctgctcagatcactgtatcacctcttcagacatgatggactcgtccagcttctccaatctggatctgagtgaagaagattcagatgacccttctgtgaccctagagctgtcccagctctccatgctgccccacctggctgacctggtcagttacagcatccaaaaggtcattggctttgctaagatgataccaggattcagagacctcacctctgaggaccagatcgtactgctgaagtcaagtgccattgaggtcatcatgttgcgctccaatgagtccttcaccatggacgacatgtcctggacctgtggcaaccaagactacaagtaccgcgtcagtgacgtgaccaaagccggacacagcctggagctgattgagcccctcatcaagttccaggtgggactgaagaagctgaacttgcatgaggaggagcatgtcctgctcatggccatctgcatcgtctccccagatcgtcctggggtgcaggacgccgcgctgattgaggccatccaggaccgcctgtccaacacactgcagacgtacatccgctgccgccacccgcccccgggcagccacctgctctatgccaagatgatccagaagctagccgacctgcgcagcctcaatgaggagcactccaagcagtaccgctgcctctccttccagcctgagtgcagcatgaagctaacgccccttgtgctcgaagtgtttggcaatgagatctcctgactaggacagcctgtggcggtgcctgggtggggctgctcctccagggccacgtgccaggcccggggctggcggctactcagcagccctcctcaccccgtctggggttcagcccctcctctgccacctcccctatccacccagcccattctctctcctgtccaacctaacccctttcctgcgggcttttccccggtcccttgagacctcagccatgaggagttgctgtttgtttgacaaagaaacccaagtgggggcagagggcagaggctggaggcagggccttgcccagagatgcctccaccgctgcctaagtggctgctgactgatgttgagggaacagacaggagaaatgcatccattcctcagggacagagacacctgcacctccccccactgcaggccccgcttgtccagcgcctagtggggtctccctctcctgcctactcacgataaataatcggcccacagctcccaccccacccccttcagtgcccaccaacatcccattgccctggttatattctcacgggcagtagctgtggtgaggtgggttttcttcccatcactggagcaccaggcacgaacccacctgctgagagacccaaggaggaaaaacagacaaaaacagcctcacagaagaatatgacagctgtccctgtcaccaagctcacagttcctcgccctgggtctaaggggttggttgaggtggaagccctccttccacggatccatgtagcaggactgaattgtccccagtttgcagaaaagcacctgccgacctcgtcctccccctgccagtgccttacctcctgcccaggagagccagccctccctgtcctcctcggatcaccgagagtagccgagagcctgctcccccaccccctccccaggggagagggtctggagaagcagtgagccgcatcttctccatctggcagggtgggatggaggagaagaattttcagaccccagcggctgagtcatgatctccctgccgcctcaatgtggttgcaaggccgctgttcacccacagggctaagagctagcgctgccgcaccccagagtgtgggaagggagagcggggcagtctcgggtggctagtcagagagagtgtttgggggttccgtgatgtagggtaaggtgccttcttattctcactccaccacccaaaagtcaaaaggtgcctgtgaggcaggggcggagtgatacaacttcaagtgcatgctctctgcagccagcccagcccagctggtgggaagcgtctgtccgtttactccaaggtggggtctttgtgagagtgagctgtaggtgtgcgggaccggtacagaaaggcgttcttcgaggtggatcacagaggcttcttcagatcagtgcttgagtttggggaatgcggccgcattccctgagtcaccaggaatgttaaagtcagtgggaacgtgactgccccaactcctggaagctgtgtccttgcacctgcatccgtagttccctgaaaacccagagaggaatcagacttcacactgcaagagccttggtgtccacctggccccatgtctctcagaattcttcaggtggaaaaacatctgaaagccacgttccttactgcagaatagcatatatatcgcttaatcttaaatttattagatatgagttgttttcagactcagactccatttgtattatagtctaatatacagggtagcaggtaccactgatttggagatatttatggggggagaacttacattgtgaaacttctgtacattaattattattgctgttgttattttacaagggtctagggagagacccttgtttgattttagctgcagaacgtattggtccagcttgctcttcagtgggagaaaacacttgtaagttgctaaacgagtcaatcccctcattcaggaaaactgacagaggagggcgtgactcacccaagcatatataactagctagaagtgggccaggacaggcccggcgcggtggctcacgcctgtaatcccagcagtttgggaggtcgaggtaggtggatcacctgaggtcgggagttcgagaccaacctgaccaacatggagaaaccctgtctctattaaaaatacaaaaaaaaaaaaaaaaaaaatagccgggcatggtggcgcaagcctgtaatcccagctactcaggaggctgaggcagaagaattgaacccaggaggtggaggttgcagtgagctgagatcgtgccgttactctccaacctggacaacaagagcgaaactccgtcttagaagtggaccaggacaggaccagattttggagtcatggtccggtgtccttttcactacaccatgtttgagctcagacccccactctcattccccaggtggctgacccagtccctgggggaagccctggatttcagaaagagcaagtctggatctgggaccctttccttccttccctggcttgtaactccaccaacccatcagaaggagaaggaaggagactcacctctgcctcaatgtgaatcagaccctaccccaccacgatgtggccctggcctgctgggctctccacctcagccttggataatgctgttgcctcatctataacatgcatttgtctttgtaatgtcaccaccttcccagctctccctctggccctgccttcttcggggaactcctggaaatatcagttactcagccctgggccccaccacctaggccactcctccaaaggaagtctaggagctgggaggaaaagaaaagaggggaaaatgagtttttatggggctgaacggggagaaaaggtcatcatcgattctactttagaatgagagtgtgaaatagacatttgtaaatgtaaaacttttaaggtatatcattataactgaaggagaaggtgccccaaaatgcaagattttccacaagattcccagagacaggaaaatcctctggctggctaactggaagcatgtaggagaatccaagcgaggtcaacagagaaggcaggaatgtgtggcagatttagtgaaagctagagatatggcagcgaaaggatgtaaacagtgcctgctgaatgatttccaaagagaaaaaaagtttgccagaagtttgtcaagtcaaccaatgtagaaagctttgcttatggtaataaaaatggctcatacttatatagcacttactttgttgcaagtactgctgtaaataaatgctttatgcaaaccaaaaaaaaaaaaaaaaaa

SEQ ID NO: 42 - DNA, 2752 bp, Homo sapiens
cgctaaggccgccctccgggtagccgccatgtcggtctccagcggcgtccagattctgacaaagccagagaccgtggaccggaggagaagtgcggaaacgaccaaagaggccgggcgaccactggagatggctgtgtccgagcccgaggccagcgccgcggaatggaagcaactggatcctgctcagagcaacctgtataatgatgtgatgctggagaactactgcaaccaagcctcaatggggtgtcaagctcccaaaccagacatgatctccaagttggaaaaaggagaagcaccatggttggggaaggggaaaagacccagtcaaggttgtccaagtaaaatagcaagacccaagcaaaaagaaactgatggaaaagtccagaaagatgatgaccagcttgaaaatatccagaaatctcaaaacaaactcctcagggaagttgcagtcaagaagaaaactcaagctaagaagaatggcagtgactgtggttcactggggaaaaaaaataatttgcataaaaaacatgttccttcaaagaaaaggcttcttaaatttgagtcatgtggaaaaattttgaaacagaatttagatttacctgatcactcaagaaactgtgtaaaaaggaaatctgatgcagctaaagaacacaagaagtcattcaaccatagcttatctgatacaaggaaaggcaaaaagcaaactggaaagaaacatgagaaattatccagccatagctcatctgataagtgtaacaaaactggcaaaaaacatgacaaattatgctgtcatagttcatcccatattaaacaggacaaaattcaaactggagagaaacatgagaaatcacccagccttagctcatctactaagcatgaaaaacctcaagcttgtgtgaaaccctatgaatgtaatcaatgtggaaaggttctcagccataaacaaggactcattgaccatcagagagttcatactggggagaaaccatatgaatgtaatgaatgtgggatagcctttagccaaaagtcacaccttgttgtacatcagagaactcacaccggagaaaaaccatatgaatgtattcagtgtggcaaagcccatggtcataaacatgcactcactgaccatctaagaattcatactggagaaaagccctatgaatgtgctgaatgtgggaaaaccttcagacacagctcaaaccttattcaacatgtgagatctcacacaggtgagaagccatatgaatgtaaggaatgtgggaagtcttttaggtataactcatctcttaccgaacatgtgagaacacatacaggtgaaataccatatgaatgcaatgaatgtggaaaagcctttaagtatagctcatcccttactaaacacatgagaattcatacaggtgagaaaccctttgaatgtaatgaatgtgggaaagctttcagcaagaagtcacacctcattatacatcaaagaactcatactaaggagaaaccttataaatgtaatgagtgtggaaaagcctttggacatagctcatctcttacttaccatatgagaactcatacaggtgaaagtccctttgaatgtaatcaatgtgggaaaggctttaaacaaattgaaggccttactcaacatcagagagttcatactggagagaaaccgtatgagtgtaatgaatgtgggaaagcctttagccaaaagtctcacctcattgtacatcagagaactcatactggggagaaaccttatgaatgtaacgaatgtgaaaaagcctttaatgcaaaatcacagcttgttatacatcagcgatcccacactggagaaaaaccctatgaatgtaatgaatgtgggaaaactttcaaacaaaatgcatccctaaccaaacatgtgaaaactcattcagaagataaatctcatgagtgaagttaatgtgggaaatttgttaactaaatttaaggttttgttgaaccttggaaatatgctgattttaaagagtgttagaaagcctttagcaagatgtcacaccttgttgtacatgagaaactttgaaaatggatcttcatatagaatcagtggatggaaacaagggtttaaacttgatacaaaaccttttataaaaaatattgttttaactactcaattattataaatgatctacatgttcagattgaaattgaaagctttaaaaattgtgaattatttgaatattcaaagcaatggagaaacaagttatatatgctgacagttcctgtgtttcttcttgttttgtacttaagcaccatatattagagttgaatttgcatatctgtttattgccttatgtaaatgagtgtggccatctgtataagcctctttgacattttcacccagctctttcttaatgtctgtgttggcttctgcatcagcagaaatctttttaaaagggttgttatatatgtaaaaaatgttaacaggaaaatgagagaaaataaagcagctgaggatacaaatgtgctggtagggagcgatattcaagatatattgtgaagtgtgaaaaagtaggttgctgaaccatgtatatagtatgttcccctttgtgtacaaaagggtatgtttgtttaacctctgtctcttatctctgtatgtgtatgtatctttgtatgcatatatacatgtatctgtatgtgaatagactttgaaaaaatacagaataaacttcatggtggttgccattgggcagaggagtataggactggggtctaggataggagcaag

SEQ ID NO: 43 - DNA, 2295 bp, Homo sapiens
gagctgcaggcggcgagcgccattgtgagcagagccggcggggcaggaggcccgggcgcctgggtccccgcggcgcgggcggagcggccggccagccaggttctgtggcctgatccccaggaggagctactctgggttaccatgagagagaccttggaggccctcagctccctgggattctctgtgggacagccagagatggccccccaaagtgagcccagggaaggatcccataatgcccaggagcagatgtcctcttctagggaagagagagcactgggggtgtgctcagggcacgaggcccctacaccggaggaaggtgcccacacagaacaagccgaggctccctgcagaggccaggcgtgctcagcacagaaggctcagcctgtgggtacctgcccaggagaggagtggatgattcggaaggtgaaggtggaggacgaagatcaggaggcagaagaggaggtcgaatggccccagcatctatcgttacttcccagcccctttcccgcgcctgacctggggcatctggctgccgcgtacaaactggagccaggggccccgggggcactgagtgggctcgcgctgtctgggtggggtccgatgccggagaagccctacggctgcggggagtgtgagcggcgcttccgggaccagctgacgttgcgactgcaccagcggctgcaccggggcgagggcccctgcgcctgcccggactgcggccgcagcttcacgcagcgcgcccacatgctactgcatcagcgcagccaccgcggcgagcggcctttcccgtgctccgagtgcgacaagcgcttcagcaagaaggcccatctgacccgccacctgcgcacgcacacgggcgagcggccctacccgtgcgcggagtgcggcaagcgcttcagccagaagatccacctgggctcgcaccaaaagacccacaccggcgagcggcccttcccctgcacggaatgcgagaagcgctttcgcaagaagacgcacttgattcggcaccagcgcatccatacgggcgagaggccctaccagtgcgcacagtgcgcacgcagcttcacgcacaagcagcacttggtgcggcaccaaagggtgcaccagacggccggcccggccaggccctctcccgactcgtccgcttctcctcattccactgccccgtccccgaccccatcctttcccgggccaaagcctttcgcgtgctccgactgcggcttgagcttcggctggaaaaagaacctcgccacgcaccagtgtctgcaccgcagcgagggtcgcccctttgggtgcgatgagtgcgcactgggcgccaccgtggatgcccccgccgccaagcccctggccagcgcgcctggcggaccgggctgcggcccaggatccgatcccgtggtgccccagcgcgccccctcgggcgagcggtccttcttctgcccggactgcgggcgcggcttctcccatgggcagcacctggcgcggcacccgcgcgtgcacacgggcgaacggcccttcgcctgcacgcagtgtgaccgccgcttcggctcgcggcctaatctggtcgcccactccagggcccacagcggcgccaggcctttcgcctgcgctcagtgcggccgccgcttcagccgcaagtcgcacctgggccgccaccaggcggtgcacactggcagccgcccccacgcctgcgccgtctgcgcccgcagcttcagctccaaaaccaacctagtccgccaccaggcgatccacacaggctcccgccccttctcctgcccgcagtgcggaaagagcttcagccgcaagacccacctggtgcggcaccagctcattcacggcgaagccgcccacgcggccccggacgccgcccttgcggccccagcctggtccgctccccccgaggtggcgccgcccccgctcttcttctgagcctagttctcacgaggaccctttcttgcccacagtttcgagaggcccgtgccatgagaccgcctggggtgagcaaggcgacctgggctgctgcccgaaggtttggccgccgcgggacacctgtttccttcccgcagtgtctgcgtccgcacagcatacccagctcggacctcctaggacagagactcagcgaacccttgctgggaaccgctgagctgaagttcttggaaggctcccacccaggtgccccgttggaaagcagatatttcccggacccagcgcggcctcaaccagggcaggaaagagtggttatttatgtacttaaagtttcattaaagttaaaatcggaaaaaaaaaaaaaaaaaa

SEQ ID NO: 44 - DNA, 4985 bp, Homo sapiens
agtccgggtcttggcgggccccctcctccctcccgctccccgcgctctcgccttttaatcatgcccctctgtctgtgtgtgagtgcaggcaggctgacaatgatttcctcagtgattacgtacagagcgagtccctgcgggttaggggccccctctggagccatcctgatggctttgggggccttgcttccattttccattattatgtggactaccggagcgacagcgcagtccaagaccttgcaggatgtctcgccgcaagcaagcgaaaccgagatccctcaaagaccccaactgtaaacttgaagacaagactgaagatggagaggcactagattgtaagaagaggccggaagacggggaggagttggaagacgaagctgtgcacagctgtgacagctgcctccaggtgtttgaatcgctgagcgatatcacagaacacaagattaatcaatgtcaactgacagatggagtggatgttgaagatgatccgacttgctcttggccagcttcctcaccttctagcaaggatcagacttcccctagccatggagaaggttgcgattttggagaggaagaaggtggccctgggcttccatacccgtgtcaattctgtgacaagtcgtttagccgcctcagctacctaaagcaccatgagcagagtcacagtgacaaactgcctttcaaatgcacctactgcagtaggctgttcaaacacaagcgcagccgagatcgccacataaaactccacaccggggacaagaagtaccactgcagtgaatgtgatgctgcgttttccagaagtgatcacttgaagatccacttaaagactcacacgtccaacaagccatataaatgtgccatttgtcgccgtgggtttctgtcctctagttccttacacggacacatgcaggttcatgagaggaacaaggacggctctcagtccggttccaggatggaggactggaagatgaaggacactcagaagtgcagtcagtgtgaggaaggctttgacttcccggaagacctccaaaaacacattgcagagtgccaccccgaatgctccccaaatgaggaccgagcggccctccagtgtgtctactgccacgagctcttcgtagaggagacctccctcatgaaccacatggagcaggtgcatagcggggagaagaagaactcatgcagcatttgttctgagagtttccacacagttgaggaactgtacagccacatggacagtcaccagcaaccggagtcatgcaatcacagcaacagcccttccctggtcacggtgggctatacctccgtgtccagtacgactccagattccaacctctcagtggacagctcaaccatggtggaagctgccccgccaatcccaaagagtcgagggaggaagagggccgctcaacaaacccctgacatgactggtccctcgagtaaacaagcaaaagttacctacagctgtatttactgcaacaaacaattattttcaagtcttgcagttctgcagattcacctgaaaactatgcacttagataagccagaacaggcccatatttgtcagtattgcttggaggtcctgccctcactctataacctaaatgaacatcttaagcaagtgcatgaagctcaggacccaggtctgattgtttctgccatgcctgccattgtctaccagtgtaacttctgttccgaagttgtcaacgacctcaacactcttcaggaacacatccgatgttctcatggatttgcaaaccctgcagctaaagatagtaatgcattcttttgtccccattgctatatggggtttctcactgactcttccctggaagagcatattagacaggttcattgtgacctcagtggctcccgatttgggtctccagtgcttgggactcccaaagaaccagtagtagaagtctattcttgttcctattgtacaaattcgccaatattcaacagcgttcttaaactgaacaagcatatcaaagagaatcataaaaacattcccttggccctgaattatatccacaatgggaagaaatccagggccttaagccccctatctcctgtggccatagagcagacatctcttaagatgatgcaggcagtaggaggtgcacctgcacgtcccactggagaatatatctgtaatcaatgtggtgctaagtacacatccctagacagctttcagactcacctaaaaactcatctcgacactgtgcttccaaaattgacctgtcctcagtgcaacaaggaattccccaaccaagaatccttgctgaagcatgttaccattcactttatgatcacttcaacgtattacatctgtgagagttgtgacaagcaattcacatcagtggatgaccttcagaaacacctgctggacatgcacacctttgtcttctttcgctgcaccctctgccaggaagtttttgactcaaaagtctccattcagctccacttggctgtgaagcacagtaacgaaaagaaagtctataggtgcacatcttgcaactgggacttccgcaacgaaactgacttgcagctccatgtgaaacacaaccacctggaaaaccaagggaaagtgcataagtgcattttctgcggtgagtcctttggcaccgaggtggagctgcaatgccacatcaccactcacagtaagaagtacaactgcaagttctgtagcaaagccttccatgcgatcattttgttagaaaaacacttgcgagaaaaacactgtgtattcgaaaccaagacacccaactgtggaacaaatggagcttccgagcaagtgcagaaagaggaagtggagctgcagactttgctgaccaacagccaggagtcccacaacagtcacgatgggagcgaagaagacgttgacacctctgagcctatgtacggctgcgacatttgtggggcagcctacactatggaaactttgctgcagaatcaccagctccgagaccacaacatcagacctggagaaagtgccatcgtgaaaaagaaagctgagctcattaaagggaattacaagtgcaacgtgtgctctcgaaccttcttctccgaaaatggcctccgggaacatatgcagacccacctaggccctgtcaaacactacatgtgccctatttgcggagagcggtttccctcccttttaactcttactgaacacaaagtcacgcatagtaagagtcttgatactggaaactgccggatttgcaagatgcctctccagagtgaagaggagtttttagagcattgccaaatgcaccctgacttgaggaattccctgacaggctttcgctgcgtggtgtgcatgcagacagtgacctccaccttggaactcaaaatccatgggacgttccacatgcaaaagacagggaatgggtctgcagttcagaccacagggcggggccagcacgtccaaaaactgtataagtgcgcatcttgcctcaaagaattccgttccaagcaagatctggtgaaacttgatatcaatggcctgccatatggtctgtgtgccggctgcgtgaatctcagtaagagcgccagcccaggcattaacgtccctcccggcacgaatagaccaggcttgggccagaatgagaatctgagtgccattgaggggaaaggcaaggtggggggactgaagacacgctgctctagctgcaacgttaagtttgagtctgaaagtgaactccagaaccacatccaaaccatccaccgagagctcgtgccagacagcaacagcacacagttgaaaacgccccaagtatcaccaatgcccagaatcagtccctcccagtcggatgagaagaagacctatcaatgcatcaagtgtcagatggttttctacaatgaatgggatattcaggttcatgttgcaaatcacatgattgatgaaggactgaaccatgaatgcaaactctgcagccagacctttgactctcctgccaaactccagtgccacctgatagagcacagcttcgaagggatgggaggcaccttcaagtgtccagtctgctttacagtatttgttcaagcaaacaagttgcagcagcatattttctctgcccatggacaagaagacaagatctatgactgtacacaatgtccacagaagtttttcttccaaacagagctgcagaatcatacaatgacccaacacagcagttagtgcaagtacagtctctcaaggagaattgattttgtggcacaaaaagggaacatgttttactctttgcacgaaactttcattgttaatgtatattattcagaaacattgtattgtaccataaaacttgtattatcaaactgttggatgttcatgtgtttgaacttttgcgcaccggatagaccccttgtatataaagtgttgcacatgtattatgtcgtctgatactaaaatggtcttataaagacaagtggacttgggccctattcaggcaagattaaaaaaaaaaaaagactatgaccaaaatggcttaagataaagtatttttaaggaagaaagattaaaaacaactgttatacatgagactatggttggacttccttttctttacacttaagcctagaatttctctttaggtatatcagcgcttaaatccaagactattttttattgctgaagattcttgcaaaccatgaagagatgttctcacagaacagaaccccacagctggataaggcccgtatatatatatttgtaagccttgcaatgtgacaggtagcatcactatatatgcaatagttgttatgtagactgtcaaagaatttttttttccctggatacatttgaagctttgagtgttcaaggttttccttaatgatttcacgcagccaaattcttgaatcagttgaactaacctgtatgttactgttattaatgtttactctgcagtctgaacctggagattactggaattgttttccaagaggaaataaattcagtttaccattaggaaaaaaaaaaaaaa

SEQ ID NO: 45 - DNA, 6493 bp, Homo sapiens
gccatgtttcaatctggccccagtggctttttctctgaaagcaaacgtgtgtcttttacaccagggctttctccccaccccagggggtgtcttccatccttttgtggctcagttgaaggcgaaaagggctccaaaccactaactaaccagaggagagccccttcttccacctccagggagaatttcagatttaatttgtccgaagatagcgtgctctcttcttactcatttgccatcattacgaggaaaacaaaccaccaccttggcttcaagatcctgggtagaggctcacggtcttttcaaccatctttggcgaggccttgcttccttccactcgaggtatgttctgtcttgtgctttttcttttagaagctactaaagggtgttggggatgcttctgactattatgaaggccaaaaggcctgttgactggggctgcttttaaccctttcctatttgctgagaatgcagccgtgtgacagtaactg[450 bp omitted]cgatctccagtgctgaagagtttgatgacgacgagaagattgaggtggatgacccccctgacaaggaggacatgcgatcaagcttcaggtcgaatgtgttgacggggtcggctccccagcaggactacgataagctgaaggcactcggaggggaaaactccagcaaaactggactctctacgtcaggcaatgtggagaaaaacaaagctgttaagagagaaacagaagccagttctataaacctgagtgtttatgaaccttttaaagtcagaaaagcagaggataaattgaaggaaagctctgacaaggtgctggaaaacagagtcctagatgggaagctgagctccgagaagaatgacaccagcctccccagcgttgcgccatcaaagacaaagtcgtcctccaagctctcgtcctgcatcgctgccatcgcggctctcagcgctaaaaaggcggcttcagactcctgcaaagaaccagtggccaattcgagggaatcctccccgttaccaaaagaagtaaatgacagtccgagagccgctgacaagtctcctgaatcccagaatctcatcgacgggaccaaaaaaccatccctgaagcaaccggatagtcccagaagcatctcaagtgagaacagcagcaaaggatccccgtcctctcccgcagggtccacaccagcaatccccaaagtccgcataaaaaccattaagacatcttctggggaaatcaagagaacagtgaccagggtattgccagaagtggatcttgactctggaaagaaaccttccgagcagacagcgtccgtgatggcctctgtgacatcccttctgtcgtctccagcatcagccgccgtcctttcctctccccccagggcgcctctccagtctgcggtcgtgaccaatgcagtttcccctgcagagctcacccccaaacaggtcacaatcaagcctgtggctactgctttcctcccagtgtctgctgtgaagacggcaggatcccaagtcattaatttgaagctcgctaacaacaccacggtgaaagccacggtcatatctgctgcctctgtccagagtgccagcagcgccatcattaaagctgccaacgccatccagcagcaaactgtcgtggtgccggcatccagcctggccaatgccaaactcgtgccaaagactgtgcaccttgccaaccttaaccttttgcctcagggtgcccaggccacctctgaactccgccaagtgctaaccaaacctcagcaacaaataaagcaggcaataatcaatgcagcagcctcgcaaccccccaaaaaggtgtctcgagtccaggtggtgtcgtccttgcagagttctgtggtggaagctttcaacaaggtgctgagcagtgtcaatccagtccctgtttacatcccaaacctcagtcctcccgccaatgcagggatcacgttaccgacgcgtgggtacaagtgcttggagtgtggggactcctttgcacttgaaaagagtctgacccagcactacgacagacggagcgtgcgcatcgaagtaacgtgcaaccattgtacaaagaacctcgttttttacaacaaatgcagcctcctttcccatgcccgtgggcataaggagaaaggggtggtaatgcaatgctcccacttaattttaaagccagtcccagcagatcaaatgatagtttctccgtcaagcaatacttccacttcaacttccactcttcagagccctgtgggagctggcacacacactgtcacaaaaattcagtctggcataactgggacagtcatatcggctccttcaagcactcccatcaccccagccatgcccctagatgaagacccctccaaactgtgtagacatagtctaaaatgtttggagtgtaatgaagtcttccaggacgagacatcactggctacacatttccagcaggctgcagatacgagtggacaaaagacttgcactatctgccagatgctgcttcctaaccagtgcagttatgcatcacaccagagaatccatcagcacaaatctccctacacctgccctgagtgtggggccatctgcaggtcggtgcacttccagacccacgtcaccaagaactgtctgcactacacgaggagagttggttttcgatgtgtgcattgcaatgttgtgtactctgatgtggctgctctgaagtctcacattcaaggttctcactgtgaagtcttctacaagtgtcctatttgtccaatggcgtttaagtctgccccaagcacacattcccacgcctacacacagcatcctggcatcaagataggagaaccaaaaataatatataagtgttccatgtgcgacactgtgttcaccctgcaaaccttgctgtatcgccactttgaccaacacattgaaaaccagaaggtgtctgttttcaagtgtccagactgttctcttttatatgcacagaagcaacttatgatggaccatatcaagtctatgcatggaacattgaaaagtattgaagggcctccaaacttgggtataaacttgcctttgagcattaagcctgcaactcaaaattcagcaaatcagaacaaagaggacaccaaatccatgaatgggaaagagaaattggaaaagaaatctccatctcctgtgaaaaaatcaatggaaaccaagaaagtggccagtcctgggtggacgtgttgggagtgtgactgcctgttcatgcagagagatgtgtacatatcccacgtgaggaaggagcacgggaagcaaatgaagaaacacccctgccgccagtgtgacaagtctttcagctcgtcccacagcctgtgccggcacaaccggatcaagcacaaaggcatcaggaaagtgtacgcctgctcgcactgcccagactccagacgtacctttaccaaacgtttgatgctggagaagcacgtccagctgatgcatggcatcaaggaccctgacctgaaagaaatgacagatgccaccaatgaggaggaaacagaaataaaagaagacactaaggtccccagtcccaagcggaagttggaagaaccagttctggagttcaggcctccccgaggagcaatcactcaaccactgaaaaagctgaaaatcaatgtttttaaggttcacaagtgtgccgtgtgtggcttcaccaccgaaaacctgctgcaattccacgaacacatccctcagcacaaatcggatggttcttcctaccagtgccgggagtgtggcctctgctacacgtctcacgtctctctgtccaggcacctcttcatcgtacacaagttaaaggaacctcagccagtgtccaagcaaaatggggctggggaagataaccaacaggagaacaaacccagccacgaggatgaatcccctgatggcgccgtgtcagacagaaagtgcaaagtgtgcgcaaaaacttttgaaactgaagctgccttaaatactcacatgcggacacacggcatggccttcatcaaatccaaaaggatgagctcagccgagaaatagccacagatgctccatgaggaaaatccctgtccacattggaataaaaaagacatttttgttacaaagtttgcagtataatagagttaacagtactgtctaggctgttgcaatatattctctttcaatgtaccttccttcacctcgtcgtatatatcctcgataagtattaaaacagtatttgagtttaaaagagtttgtatatatttaaatgaataactttttatactctttgttacatgtttgtatcagtatttagtggaaaaccatttgagttgttttgggttagaatttttctttttgtactgtttctttaaaacagagttcttagtaacaggggcagttcctgaattcaaataaaccattttgtatgtttggattttgaatgggttaactaattacaggctaaaataatgccttttttagtgtttttaatttttagaattcactacataaattgtaagtaattgtgggtctcaaaaacactaggaacttttaagtgtcttagcacttcctcgatgtgcctgccctgagggagtgagttcacatttgagacaactgcactccagtgtggacgtgcctttgtcttcaggccatgccgaagggtgtttaaagcagtcttgcaggtcgctcctttcccagccgtggataaaaactgaagctaggaatctaataaggaatgctgatttcctcagttccattttgaggaatggggaaggctattctaaagaaaaaaatgggatttgttttctcggcagatctgcaaggctggctttaagagcacaaggagggaaagtaacgaaagggctggactactataaaagttacaaatacgtagttagaccaatagatttatatagtcaggtttttgtcatgtaatttattaactaactattacagaaacacagctaagaatatcaagtatttctctggctcttgacagaaaaaaatcagttgacttaaccctttgctgtcaaaagagttggcgtttcctgttctgggtgctactgccaaacgttatggtacttagagtcgggatgcacaacttcaaccaccgacttatcaatgcagccgcctgtgtattgcaattggccgttaccttaagcactgagccacccgggtttagttcagccatttcaagaagtatatttaacgtcggtagttctgctttattaaaatgcagcagaggtactcttctgtcccttccgtttatagttctctgagagagttctattttttggttttgttttgtgttttcttttgcattttgtatcttgtatttatccctgaacatgttttgtaccttttttttttttttttttaagaaaaggaattcttttgtgtatatatagatacttgcatgatatactgtagtcaatgttcggttcctcaaaaggtcttgctgctgtcaggtgttatgcactccatccatcataactgtatgaaacacatttcatatgtaaataaacgtgggacatttggcccttgtgcttctgtgagagaattattgatggtgggtctctgacatctttgtgaagtttgggaagtaattaattgcagcgacaagctacagggtgttgcagaattcttcccactcagaagaatggcatattcgttctcattagtaatcagctattttgtcactttcttgttgactccatcagtacatgggtacaatccgagggtgtgaatttcagcttgaaattccattgctgttccttgttttgtttgtattgctctaagttgtattcataatagcactttcatatgtttctgcatttgaaccttgcaataagcctgtgtggtaggccacataggtccgaataacctagttttacagttgagggagctgagctcagattcagttctttgccgaagccctcatagctggtaagtggctttgcatattagaacccaaatattttgctctctaaatctaatgctcgctctatgtggttatgtacatattgacaaatattcatttattcaacaaataaaaagtatgtacaaaacaaaaaaaaaaaaaaaaaaaaaaaaaaaaaa

SEQ ID NO: 46 - DNA, 3271 bp, Homo sapiens
cggaggtgagtgcagggctccgggcgctgtgtgccgtgccctgtcccccaggctctgggtccggttgagacgcactcccaccctgggtgctgcagggtctcccgtggcctacgctgccaggaggcccaggctgggagagggagcagggcggcggcccggggtgctcttccgcgctcctcgactccagctttggtctttgtggctcaatagcttgcgcccattggaaggggaaacgcacgttgctgtcccagccacgaggccctgagcatttagcacttggtcgacgggggaagatgtgtcggtgcagctaaaagatggaatatttgcgggcgtgaagcggcccgaggtgataccacaagtcttttgacatgctgaggtctggcaggaacattaacctatgtgcatcccgagtatggaccgtgtgctcccaggctcctgacatagggtcatgaattagggccgagtgggagcgcagagcccctcccagtcacccggcagcagaagcagcccggcttttggaggacattgtctcctggagcagtgtcagtcccaaaagctccagcccttctctgagacggggaccaggggatggcagccatgcacctgacagcctggccccagggacttcacggatcccctgaagctcatccatggacttccatggactacagggcctgagctatggaattggccctagggcagcaggaacctattgtttcagaagtcggtgacctttgaggacgtggctgtgtacttcacccaggcggaatgggatggcctgtcccctgcacagaggaccctgtacagggatgtgatgctggagaattatgggaatgtggcctccctgggatttccacttctcaaacctgctgtgatctcacaactggagggaggaagtgagctggggggctcatctccactggctgcaggaacaggcctccagggcctccagactgatattcagactgacaatgatttgacaaaggaaatgtatgaaggaaaagagaatgtatcatttgaacttcaaagagacttttcccaggaaacagacttttcagaagcctctcttctagagaaacaacaggaagtccactcagcaggaaatataaagaaggagaagagcaacaccattgatggaacagtgaaagatgagacaagccccgtggaggagtgtttttttagtcaaagttcaaactcatatcagtgtcataccatcactggagagcagccctctgggtgtacaggattggggaaatccatcagctttgatacaaaactcgtgaagcatgaaataattaattctgaggaaagacctttcaaatgtgaagaattagtagagccctttaggtgtgactctcaacttattcaacatcaagagaacaacactgaggaaaagccttatcagtgttcggagtgtggcaaagctttcagcattaatgagaaattaatttggcatcagagacttcacagtggggagaaacccttcaaatgtgtggagtgtgggaaaagcttcagctacagttcccattatatcacacatcagacaatccacagtggggagaagccctatcagtgtaagatgtgtgggaaggccttcagtgttaatggaagcctaagtaggcatcagagaatccatacgggagagaagccctatcagtgcaaggaatgtggaaatggcttcagctgtagttctgcatatattacacatcagagagtccacactggagagaaaccttacgagtgtaatgactgtgggaaagcgttcaatgttaatgcaaaattaattcaacatcagagaatccatactggagagaaaccttatgaatgtaatgaatgtggaaaaggcttcaggtgcagctcccagcttaggcagcatcagagcatccacacaggagaaaagccctatcagtgtaaagagtgtggaaaaggcttcaataataatacaaaactcattcagcatcagagaatccacacaggtgagaaaccctatgaatgcactgaatgtggaaaagccttcagtgtcaaagggaagttaatccaacaccagagaattcacacaggcgagaaaccctatgagtgtaatgaatgcgggaaagccttcagatgtaactcccaatttcggcagcatctgagaattcacactggggagaagccctatgagtgtaatgagtgtggaaaggccttcagcgttaatgggaaactaatgcggcatcagagaattcacactggggagaaaccttttgaatgtaatgagtgtgggagatgctttacttctaaaagaaacctacttgatcatcaccgaatccatactggagaaaagccctatcaatgtaaggaatgtgggaaagccttcagtatcaatgccaaactaactaggcatcagaggatacatactggggagaaacctttcaaatgtatggaatgtgagaaagcattcagctgtagttctaactatattgtgcaccagagaatccatacaggagagaaaccctttcagtgtaaggagtgtggaaaagccttccatgttaatgcccatttaattcggcatcagagaagccacactggggagaaacccttcagatgtgtggaatgtggcaaaggcttcagctttagttctgactacattatacatcagacagtccacacttggaagaaaccctatatgtgtagtgtgtgtgggaaagcattcaggtttagcttccagctcagtcagcatcagagtgtccatagtgaaggaaaatcctaataatgagaaagatacagaaaactcttaaggttaatgccaaaatggatcaagtatcatcagattcatccattgaaaaacctccaagagggcatgaatatggcagagtcttcatatggaaacagtttttattctattcagtttaaatcaggaaaggatgaccagttaaagagaaacatccaaaaatagctttgttttgtaccaacaggaattagaaaatataatgaaaagatttcgttcccagcagcatcaagaaaagtagattttctagaaataaacagttatggaggacttgtatggagaaatttaagtcttcactgagggccactttacaaaggaaatttgaataaatggagagagagagaagccttgttgttggataggaaaacccgtactaaagatactctacctacattaatttatttgtttaatttttgacaacaagcatgtattacttttgaaaagatgaaaaataaagatttatttaaaaaaggaaaaaaaaaaaaaaaaaaaaaaaaaaaaaaaaac

SEQ ID NO: 47 - DNA, 1952 bp, Mus musculus
gcgcgggggtggggccagcgcgtataaaggggcgcagcgggctgggcgttccacaggccgagtgcgctgtgctcgaggggtgccggctaggcccgactgagagcaagcgaacaggcaggcaagctccgagggggcgcggcccaagctggacaggacaagcgatccagacgcaggagccgtccatcaccaatcagccagccttcgaccatgggcatgtccgacgtgtacctccgcagcagaacagcgatggaacgcttggcctccagcgataccttcccagtgatagcgcgtagcagcgcctgccgcagcctcttcgggcctgtagaccacgaggagctgggccgcgagctgcggatgcgcctggccgagctgaacgccgaggaccagaaccgctgggacttcaacttccagcaggatgtgcctcttcgaggccctggtcgtctgcagtggatggaggtggacagcgagtctgtgcccgccttctaccgcgagacggtgcaggtggggcgctgtcgcctgcagctggggccccggccacccccggtggccgtggctgtcatcccgcgttctgggccgccggctggcgaggcccccgacggcctagaggaggcgcctgagcagccgcccagcgccccagcctcggccgtggtcgcggagcccaccccacccgcgaccccggccccggcttcagatctgacctcagacccaattccggaggtgaccctggtcgcgacctccgacccgactccggacccgatcccggacgcgaacccggacgtggcgactcgggacggcgaggaacaggtccctgagcaggtctctgagcagggcgaggagtcgggtgctgagccgggtgatgagctgggaactgagccggtctctgagcagggcgaggagcagggcgcagagccggtcgaggagaaggacgaggagccggaggaggagcagggcgcggagccggtcgaggagcagggtgcggagccggtcgaggagcagaatggggagccggtcgaggagcaggacgagaatcaagagcagcgcggccaggagctgaaggaccagcctctctcggggattccaggacgtcctgcacccgggactgctgcggccaatgcgaacgacttcttcgccaagcgcaagagaactgcgcaggagaacaaggcgtcgaacgacgtccctccagggtgtccctctccaaacgtggctcctggggtgggcgcggtggagcagaccccgcgcaaacgtctgagatgagttagtttagaggctaacggccagagagaacttgctgggcatctgggcagcggacgatggaagaactctgggcttcggctgggacctttcgttcatgtagcaggaaccggagatggttgcgtagagcagcccacggttttgtggaaatctgaaaactgtgcaatgtattgagaacactctgtaccatgtgcaaggagtacgctggtcccaaggtgtaaagctttaaatcatttatgtaaaatgtttaatctctactcgctctcagtgcaaaacaaaaagagaaactagaaaatgtagaacgaaggaaaaagatgagaaaaaggaaaaagcatgtatatttgtacaaaaagttaaaaaattatgctaatttaatatttgtatttatccatgcgtggatccctctgccacgcaactgctgggttattgattattaccaaaggcactagaaatcaccagcttcagattacccacaaatgtaaatctacttttatattagactctaggggaatggttgttgagtaaaagcccccacacattcatcttcagttagcttacagtgtcccccacttcattagattgctcttaaacttggtgaccagacagtcgaaatggttcctgtactgcccataccaactgcccagggctcctcgtccgccaggttctaaataaagaggcccaacaaaaatcaaaaaaaaaaa

SEQ ID NO: 48 - DNA, 4281 bp, Mus musculus
cacccactcccgtgcgcgcccggcccgtagcgtcctcgtcgccgcccctcgtctcgcagccgcagcccgcgtggacgctctcgcctgagcgccgcggactagcccgggtggcccactggcgcgcgggcgagcgcacgggcgctccagtccggcagcgccggggttaagcggcccaagtaaacgtagcgcagcgatcggcgccggagattcgcgaacccgacactccgcgccgcccgccggccaggacccgcggcgcgatcgcggcgccgcgctacagccagcctcacgacaggcccgctgaggcttgtgccagaccttggaaacctcaggtatatacctttccagacgcgggatctcccctcccccatccatagtgccttgggaccaaatccagggccttctttcaggaaacaatgaagggagacagcagacatctgaatgaagaagagggtgccagcgggtatgaggagtgcattatcgttaatgggaacttcagtgaccagtcctcagacacgaaggatgctccctcacccccagtcttggaggcaatctgcacagagccagtctgcacaccagagaccagaggccgcaggtcaagctcccggctgtctaagagggaggtctccagccttctgaattacacgcaggacatgacaggagatggagacagagatgatgaagtagatgatgggaatggctctgatattctaatgccaaagctcacccgtgagaccaaggacaccaggacgcgctctgaaagcccggctgtccgaacccgacatagcaatgggacctccagcttggagaggcaaagagcctcccccagaatcacccgaggtcggcagggccgccaccatgtgcaggagtaccctgtggagtttccggctaccaggtctcggagacgtcgagcatcatcttcagcaagcacgccatggtcatcccctgccagcgtcgacttcatggaagaagtgacacctaagagcgtcagtaccccatcagttgacttgagccaggatggagatcaggagggtatggataccacacaggtggatgcagagagcagagatggagacagcacagagtatcaggatgataaagagtttggaataggtgacctcgtgtggggaaagatcaagggcttctcctggtggcctgccatggtggtgtcctggaaagccacctccaagcgacaggccatgcccggaatgcgctgggtacagtggtttggtgatggcaagttttctgagatctctgctgacaaactggtggctctggggctgttcagccagcactttaatctggctaccttcaataagctggtttcttataggaaggccatgtaccacactctggagaaagccagggttcgagctggcaagaccttctccagcagtcctggagagtcactggaggaccagctgaagcccatgctggagtgggcccacggtggcttcaagcctactgggatcgagggcctcaaacccaacaagaagcaaccagagaacaaaagtcgaagacgcacaaccaatgactctgctgcttctgagtcccccccacccaagcgcctcaagacaaatagctatggcgggaaggaccgaggggaggatgaggagagccgagaacggatggcttctgaagtcaccaacaacaagggcaatctggaagaccgctgtttgtcctgtggaaagaagaaccctgtgtccttccaccccctctttgagggtgggctctgtcagagttgccgggatcgcttcctagagctcttctacatgtatgatgaggacggctatcagtcctactgcaccgtgtgctgtgagggccgtgaactgctgctgtgcagtaacacaagctgctgcagatgcttctgtgtggagtgtctggaggtgctggtgggcgcaggcacagctgaggatgccaagctgcaggaaccctggagctgctatatgtgcctccctcagcgctgccatggggtcctccgacgcaggaaagattggaacatgcgcctgcaagacttcttcactactgatcctgacctggaagaatttgagccacccaagttgtacccagcaattcctgcagccaaaaggaggcccattagagtcctgtctctgtttgatggaattgcaacggggtacttggtgctcaaggagttgggtattaaagtggaaaagtacattgcctccgaagtctgtgcagagtccatcgctgtgggaactgttaagcatgaaggccagatcaaatatgtcaatgacgtccggaaaatcaccaagaaaaatattgaagagtggggcccgttcgacttggtgattggtggaagcccatgcaatgatctctctaacgtcaatcctgcccgcaaaggtttatatgagggcacaggaaggctcttcttcgagttttaccacttgctgaattatacccgccccaaggagggcgacaaccgtccattcttctggatgttcgagaatgttgtggccatgaaagtgaatgacaagaaagacatctcaagattcctggcatgtaacccagtgatgatcgatgccatcaaggtgtctgctgctcacagggcccggtacttctggggtaacctacccggaatgaacaggcccgtgatggcttcaaagaatgataagctcgagctgcaggactgcctggagttcagtaggacagcaaagttaaagaaagtgcagacaataaccaccaagtcgaactccatcagacagggcaaaaaccagcttttccctgtagtcatgaatggcaaggacgacgttttgtggtgcactgagctcgaaaggatcttcggcttccctgctcactacacggacgtgtccaacatgggccgcggcgcccgtcagaagctgctgggcaggtcctggagtgtaccggtcatcagacacctgtttgcccccttgaaggactactttgcctgtgaatagttctacccaggactggggagctctcggtcagagccagtgcccagagtcacccctccctgaaggcacctcacctgtcccctttttagctcacctgtgtggggcctcacatcactgtacctcagctttctcctgctcagtgggagcagagcctcctggcccttgcaggggagccccggtgctccctccgtgtgcacagctcagacctggctgcttagagtagcccggcatggtgctcatgttctcttaccctgaaactttaaaacttgaagtaggtagtaagatggctttcttttaccctcctgagtttatcactcagaagtgatggctaagataccaaaaaaacaaacaaaaacagaaacaaaaaacaaaaaaaaaacctcaacagctctcttagtactcaggttcatgctgcaaaatcacttgagattttgtttttaagtaacccgtgctccacatttgctggaggatgctattgtgaatgtgggctcagatgagcaaggtcaaggggccaaaaaaaattccccctctccccccaggagtatttgaagatgatgtttatggtttaagtcttcctggcaccttccccttgctttggtacaagggctgaagtcctgttggtcttgtagcatttcccaggatgatgatgtcagcagggatgacatcatcacctttagggcttttccctggcaggggcccatgtggctagtcctcacgaagactggagtagaatgtttggagctcaggaagggtgggtggagtggccctcttccaggtgtgagggatacgaaggaggaagcttagggaaatccattccccactccctcttgccaaatgaggggcccagtccccaacagctcaggtccccagaaccccctagttcctcatgagaagctaggaccagaagcacatcgttccccttatctgagcagtgtttggggaactacagtgaaaaccttctggagatgttaaaagctttttaccccacgatagattgtgtttttaaggggtgctttttttaggggcatcactggagataagaaagctgcatttcagaaatgccatcgtaatggtttttaaacaccttttacctaattacaggtgctattttatagaagcagacaacacttctttttatgactctcagacttctattttcatgttaccattttttttgtaactcgcaaggtgtgggcttttgtaacttcacaggtgtggggagagactgccttgtttcaacagtttgtctccactggtttctaatttttaggtgcaaagatgacagatgcccagagtttacctttctggttgattaaagttgtatttctctataaaaaaaaaaaaaaaaaaa

SEQ ID NO: 49 - DNA, 2214 bp, Mus musculus
gttgttctaagatctttggtgatccaagaaaagctgtgttggtcaagggcaggacagatgactcccagagaaaaaagatggcgaaaccaaggacaatcagagtcgtcccgggttctgcatggctggtgagtgagctggcctgctgaaggccatgatccagactgtacctgacccagcagctcatattaaggaggccttgtcagttgtgagcgaggaccagtcactatttgagtgtgcctacggaacgccacacctggctaagacagagatgaccgcatcctcttccagtgactatggccagacatccaagatgagtcccagagtccctcagcaggactggctgtctcaagccccagccagggtcaccatcaagatggagtgcaaccctagtcaggtgaatggttccaggaactcacctgatgagtgcagtgtgaacaaaggtgggaagatggtgggcagcccggatactgtggggatgagctacggcagctacatggaggagaagcatgtgccgcctcccaatatgaccacaaatgagcgcagagtgatcgtccctgcagatcctactctgtggagcacagaccatgtccgacagtggctggagtgggcggtgaaagaatatggcctcctcgatgtggacgtcttactatttcagaatatcgatgggaaggagctgtgcaagatgacaaaggatgacttccagcggctcacgccgagctacaatgccgacattcttctctcacatctccactacctcagagagactccccttccacatctgacttccgatgacgttgataaggctttacaaaactctccacggttaatgcatgccagaaacacagggggtgcagcttttattttcccaaatacttcagtatatcccgaagctacgcaaagaattacaactaggccagatttaccttatgagcctcccaggagatcagcctggaccggccacagccacctcacccctcagtccaaagctgctcagccatctccctctgcagtgcccaaaactgaagaccagcgtcctcagttagatccttaccagatcctgggaccgaccagtagccgccttgctaatccaggtagtggccagatccagctgtggcagttcctgctcgaactcctgtcagacagctccaactccaactgcatcacctgggaaggcaccaacggggagttcaagatgacagacccggacgaggtggctcggcgctggggggagaggaagagcaagcccaacatgaactatgacaagctcagccgcgccctccgctactactacgacaaaaacatcatgaccaaggtgcacgggaagcgctacgcctacaagtttgacttccacgggattgcccaggccctgcagccccaccctcctgagtcgtccctgtacaagtacccctccgacctgccatacatgggctcctatcacgcccacccccagaagatgaactttgtgtctccccaccctcccgctctcccagtcacatcttccagtttctttgcttccccgaacccatactggaattcaccgactgggggcatctacccgaacactaggctcccagccagccatatgccctctcacctgggcacctactactagagaccagatggaggcctttcccaacagtgtacaatcgcctagcagtcgccacaaactcagcgaacacaaatcaaaagtgcctcgtgaggagtggaaacggttggccagagctggggaaggaagctggggggggaaaaaaaaaccaaagactcgcaggggagggtgggtgccacccaagtattactacagaaatagaagaagctcaaaatatactgtacatggacatataacctgtggtccacccttgtcaaagacattgtatgtagaaaagcttgaccaaaaagaacaactgcccaaggaagtggccttaagtagtgtacaaactgtagagtgtgggaccttgcttgacctaccaggattgagtgaaagcaatagaacaggaacagacttggcctaacacaccatgtatgatgtcatttgaagggaaactacctgtgtttaaaaatagaaaccatgtcaaaaccaaagaagaagcagacaaaaacagagatggcgggcccctccacagaccacaatggctgtggactttcttggctggcatttgcttgttttctttgaaatcagattcgttccatttgatagagagctgccagctctgtcaa

SEQ ID NO: 50 - DNA, 5541 bp, Mus musculus
tttcagtttctctcttccaggaaggaaaacattcgagaaagagagagggagggagggagcgagggagcgagagagcgagcgagcgagcgccgcgggagggcgggcgcgccggccgcgggtgggaggagacacgccaggccggcctgctgcgcccgggtcgccgcgccgcgacccgcacaccccacgccgtgctcgccggctcctacgcccccgcacctcgcgcccaatccgcgcgttgagaagagccccccctccccctcgggcggccgccgccagcgatgctgcaagaaacttcttaaacgaccgcatcccgctgccccgccgagcgtttctgggttggggagaggaaaggaaagtggaaaaaaactgagaacttcctgatccctttcgctgtgagacatgtctgagactcctgctcagtctagcattaagcaggaacgaatttcatacacgcccccagagagtccagtggcaagccaccgttcctcgactccgcttcatgttcacacagtgcctcgagcgctcaggatggaggaagactcgatccacctgccaacacacctgcgtttgcagccgatttactggagcagagatgacgtagcccagtggctcaagtgggcagaaaatgagttttctttaaggcccattgagagcaacaagttcgaaatgaatggcaaggccctcctgctgctgaccaaagaggatttccgctaccgatctcctcattcaggcgacgtgctctatgaactccttcagcatatcctgaagcagaggaaatctcgaatgctcttctcaccattcttcccccctggggactctatccacaccaagccagaggtcctcctgcatcagaaccatgacgaagataactgtgtccagaggacacccaggacgcccgcggagagcgtgcaccacaaccctcccaccatcgaactcttacatcgccctaggtcacccatcaccacaaaccacaggccttctcctgaccccgaacagcagcggccccagcggtcccccctagacaacatgagccgccgcctctcgccagtggagaaagcccaggggcccaggctacagcaggagaacaaccaccaggaaacgtaccccctgtcagtgtctcctgtcgagaataatcactgcctgccctcaagcccctggcaggagagcactcgagtgatccagctgatgcccagccccatcatgcaccctttgatcctgaacccccggcactcgcactcggtggacttcaaacagtcccggcactccgaggatgggatgaatcgggaagggaagcccatcaacctgtctcatcgggaggacctggcttacttgaaccacatcatggtctctatgtccccaccggaagagcacgccatgcccattgggagaatagcagactgtagactgctttgggattatgtctatcagttgctgtctgacagccggtacgaaaacttcatccgatgggaggacaaagaatccaaaatattccggatagtggatcccaacggactggctcgactctggggaaaccataagaacagaacaaacatgacctatgagaaaatgtccagagccctgcgccactactacaaactaaacattatcaggaaggagcccggacaaaggcttttgttcaggttcatgaaaaccccagatgagatcatgagtggccggacagaccgtctagaacacctcgagtctcaagtgctggatgaacaaacgtaccaagaggatgaacctaccatagcctcaccggtgggctggccaagaggaaacctgcccacggggaccgcaggaggcgtgatggaagcaggcgagctaggggtggctgtaaaggaagagacccgggaatagcagggacacttctcctgccgatcaagagggacccagagcaccttagacaagccaccgagcaatggcagggctgaagttctggcggagggcacaagcctgagactcacacgtcacgttcgcttctccttctgatctcttgtctgtaactctcaccctctcccttcccctacacctgttgtagtctcatggtgtttctggtttcgttttttgtttttgttttttaagaacatgcagtttgactattcattgttcatacagggaagacatcacatgttgttttcctatggaaatatatctattatatatatattattaatttttttgttgttgttgcaaatctcaccaagtacggccagcttggctggtcaggaaagagaaaacttgcagaaggaatcaggttcctctttttcctgccacatagatctggtgtgtcctgtccaagtcaggtcttagatgagaaaggaaaacaaaatgagagagatactttcaaacaatggaaaagaagagttctgtctccatctccctctctcgccgctcatccccgcccctttctccttcccccttctctgcctgctatgcacttcacaggcttttcatcagagctagcctcactagcacctgaactacacagtgacccggctgctctgtgacagtcagaccccttgggcacccatctgaggaaggccagacatctccccccaccaaatgctgctgtattctagaaagaacaatgggaggatcgagcataagataggacagagggtggccacaggatttgcaaggcccaggcagtttgggattgtcctgaaagtgctacacattaggaaaatatgagagaccacacaggcctggtagtttaggagcaagaacaggtcaccaatgttgtcccaagacatcatgcccttgtgggattgtcaccaccccacacacacactccaagtcctagcctttttcttctatgctggtcctcatggaacacagaacacacatgagggaggatgctggaatttagtctcaggcacagccggtatttgcgcagacaggaaattcctcagatttagactttttccttttttccgtctgtcatggctgtatttcaggaggacatatctgtggctcttctccacctcgacataaggtcagtagcatcttccaagatgagggtaatgctattccttaggatttttgatagcagggattttattttatttatttatttatttttgggatttttttttttgagacagggtttttctgtgtatccctggaattcactctgtagaccagactaggactcagagatctacctgcctctgcctcccaagcactgagattaaaggtgtgcgcccccatgcttagctttttttccttttctagccatctaagtggactctttgaagttcactgttttaaaacccagataatgaggcctgcctttcaaaaccaccccttgtccttcctgtgttctcccacatcatggaatgagcctctcccagccctcctgggtgtgtctctgcttggtcagacgcaggacacaggtgaggctgacagaccagagctgacctacaactactcctaagtggggcacatagaggctaggaggacctggacaagtcactctcccgacgtcagctcacaggcggactgtggttcacagacactggaattgagttgactgggtatctttagtcttgctgtgacttccccgtgcctcccccacaacccacaatctgccttcaaagaggtcagtgacctgggtgaggggttccctcttaatattcatggggaatgatgagagtcacgtgctatgtgtttcttgaaagcatgacaggggttcctttttttggcctctaaaaatgactggtaccttatatcaaatggggagattaagccaaatacttgtgttgtatgagccactgggaggaagagcaagtgagaaagggtgtgctttttgtttttgttttgactgaggtcctttgtgtgaatataaaatgagaaactacaacacttcatactggccactttgcccatgcgtagaactcagtcctttaactgcccaaagaattgtccatatgaactgtgtaaggaaattcctctgttccctaaggagtcttggatgacagctttggccacttacgttagaaatgctcatggcagtgccgtctttatttcacacctggtgttgccataattgatttctttgattctcttacctgcaaaagcccttgtttgttttttggagagaaaatatggccagggttgtctgggttgtcagggttgtgcagtgctctcaggcttcagactcacagaatttagatttagccaatcagaccttggaggaaacaccacaatgggggtcctgttttgtctttttggtttttgagttttggttttttgtttgtttgtttttttacaatcaactatgatcttctagatcgacattaaagcgacattaagttcaagaggtgatagaaggaatctaaaagctcctttagtggcctttttctgcagccattgtgggttgagtcaggaaaggaatttgcagggaagcattgccatgtggaggatgggcattgccacctaggtcctgggattctgtcctgagtcctcggccgggctgtcttcaggtctccttcagcagaaaccaggggcagctggtcagatgttgacagcagcttctcttccgataattttcatccaaggccacttagtgccacttggagacagggtccctgatgtcccatatctggttttggaaagagaatggtcctcataagaaaagaacaaaacaggcttcttgcctagtacattcaggtccctctgatccagcatctttttacactaataccttgtccagataagaatgtcactccagctgacatctagatgtctgtgaggagtccctggaatgaccactcttgtcttttcacactgttcgggatccaaccataaacacagtttggcccattcactgttgttggcagaagacaacctgagagaggtcgtgtcactttccgtgggagcagacagcatgacacacagttgggatcagtatctagccggcctgcacttagcagcaggataagcgttcagaaggcagttagaaattattctggtttaaacccccatttgatccatccccggcaaagagacgaaagatgccaagggtggtttttactgcccagtcattcggtgctagttagagctgctgtgcgttctcagcctgctcctcttctgaacagaaaccaaaattggacgtcatgagaaacccagtggagcaggggtacagagggacaccccagaaccctgcctatcaacaaggcacacgggactcacccagcactcttttggccccatccgcatcaacactaaacaacttcaaatgcgcccgactcttttcaccctcacaggtgaaccctcttctggagacttctctttgtgacggcacccacctgcaagccagatgaatctagaatgactttttgttgttgttgttgtttagtttctaatctcttgtttatgaggtgtggggtttataagggactgaatcaaatgaatgtaacaaaaaaagaaaaaaaaacaaacaaaaaatgccttttctcagggccagtgagttgcaaataatttttaaagaaaaacctataattacatcatctcaataaattttttataaaaaaaaaaaaa

SEQ ID NO: 51 - DNA, 4429 bp, Mus musculus
gtttctttctggatggccgagcagatcccctttaaagagacagttcatgaaatagaaaccctgcggctgggcgcggagtggctaaaggggacgagccggtgagggtctggccccgagaaggctctgggaggacctggggggccgtcctggcctggcaggccccctaccttcgcacactttcctcctgcgggtcttgcccctggagctgcgacagaggagtgggagaagttttagtaggtttcagataactttcattacacatcgggctgataagagcaagagaaagtgaggaaagagggaggtagtgagaccagaaggaatagctgcgagctcatttaggaagaggggaaaaacccaaatacaccaaacccggctcaattctggtcttaaaacactgttggcggacaataaatccgaaacgcgtggtcctggcgatcagctcccagggaacgacaaacttatcagacacccatttggaagtggagacacgaggcttttattttaaaaaaaaatttttttttcttttaaaatatctcgaaatgttagcgggtgttctttgaaaagattttccaactcgagtacctggctgctgctgatcttatttttgtttaattctgctgtgattgcttttgattgctgagttgaggccgtagaaatcggaagatcttagatgagttttgcaatgtgaagttctgcatagatgccagtcaaccagatgtaggaagctggctcaagtacatcagattcgctggctgctatgatcagcacaaccttgttgcatgccagataaatgatcagatattctaccgagtagtcgcagacattgcgcctggggaagagctcttgctgttcatgaagagtgaagaggacccgcacgaacccatggcgcctgacatccacgaagaacggcagcaccgctgtgaggactgtgaccagctctttgaatccaaggcagagctagccgatcaccagaagttcccatgcagcacacctcactcggccttctccatggtggaggaggacttgcaacaaaacctggagagtgagagcgatctccgagagatccatggcaaccaggactgtaaggaatgtgaccgagttttccccgatctgcaaagcttggagaagcacatgctgtcacatactgaggagagggaatacaagtgtgatcagtgtcccaaggcatttaactggaagtccaatttaattcgccaccagatgtcacatgacagtggaaagcactatgagtgtgaaaactgtgccaaggttttcacggaccctagcaaccttcagcgacacattcgatctcagcatgttggtgcccgggctcatgcttgccccgagtgtggtaaaacatttgccacttcgtcaggcctcaaacagcacaagcacatccacagcagtgtgaagccctttatctcattctctcaatcaatgtacccatttcctgatagagacttgaggtcgttacctttgaaaatggagccccaatcaccaagtgaagttaagaaactgcagaagggaagctctgagtccccttttgacctcaccactaagagaaaggatgagaagcccttgacttcaggcccctcgaagccttcaggaacaccagccacaagccaagaccagcccctggatctaagtatgggcagtaggggtagagccagtgggacaaagttgactgagcctcgaaaaaaccatgtgtttggggaaaagaaaggaagcaacatggatactaggccatcttcagatggctccttgcagcatgccagacccactcccttcttcatggaccccatttatagagtagagaaaagaaagttaactgacccgcttgaagctttgaaagaaaaatacttgagaccttctccaggattcttgtttcacccgcaaatgtcagcaattgagaacatggcagaaaagctggaaagcttcagcgccctcaaacctgaggccagcgagctcctgcagtccgtgccctccatgttcagcttccgagctcctcccaacaccctgccagagaacctgctgcggaaggggaaagagcgctacacctgcaggtactgtggcaagatatttccaaggtctgcgaacctaacacggcacttgagaacccacacaggagagcaaccttacagatgcaaatactgtgatagatcattcagcatttcttccaacctgcagcgacatgtgcgcaacatccacaacaaggagaagccatttaagtgtcatttatgtgacagatgttttggtcaacaaaccaatcttgacagacacctgaagaaacatgagaacggcaacatgtctgggacggcaacgtcctcgcctcactcagagctagaaagcgcaggcgcaatcctggatgacaaagaagatgcgtactttacagagatccgcaatttcatcgggaacagcaaccatggtagccagtctcctcggaacatggaagagaggatgaatggcagtcacttcaaggataaaaaggctttggcaaccagccaaaattcagatttattggatgatgaagaagtagaagatgaggtgttgttggatgaggaggatgaagacaatgatattcctggaaagcccagaaaggagctaggggtgactcgtttagacgaggagatcccggaggatgactacgaagaagctggtgccctggagatgagctgtaaggcgtccccggtgaggtataaagaggaagactataaatctggcctttctgctctagatcacataaggcacttcacagatagcctcaaaatgagggaaatggaagagaatcaatacactgacgctgagctgtcctccattagttcttctcatgtgccagaggagcttaaacagacgttacacagaaagtccaaatcacaggcatatgctatgatgttgtcactgtctgacaaggattccctccatcccacctcccacagttcctccaacgtgtggcacagcatggcaagggctgcagcagaatccagtgccatccagtccataagccatgtatgacattgtcaaggttgaccagagtgggaccaagtccaacggtagcatggctctttcatacagaacaatttacaagactgctgagcaggatgccttataaccctgaagggtcacgcatttaaagtctggtgaccttaaactgaatgagtaaagaaagagagagaaaaaggaagctatttattctcaatattttgttttgcacagccaaggcagctgctgacttctggaggatcaattaatccaaaatgattggaggggaaaggaaacctcaccagggaaggcatctttcattccccctgccgcagggcatgggtggctgagaggagcagttgaaatggcagcattgatataaatggacatttcatagaaatcaaactctactctacaggatcacctgatctgactgggaacagtggcttctaactaccagattttttttttcctttttaaagttttatgtaatttaatcttttgcagatggaagtagtcagaagaaatgcacaatgattataggaagtgatagcaggattttttgtcaccccccacaccctcttaactttggccttcttgagtacattgtttaaaactagggggaaaaaagggtatgtgtatattgtaaactatggatgctcatgcagagaggttaagtcggtgacataactgtttatcaccactgtaccactaatacaatgtttgccaaatccttgtaatgacatcttaattttagacaatcatgtcactgtttttaatgtttcaatttttttgtgtgttgtgtgtatcatgtatttatttgttggcaaactatggtttgttgatcaaaaagagcactgttcccgtcagccactattttatgatgtctgaggcacacccctttctgaattccaaggaccaaggtgacatgacctgtgtatgggagtgcccaatagtgtttggcttttcttaacattccttcctttttgttgttgttgttttgttttcctttttaatgaactaaataccaatagatgcaacttagtttttgtaatactgaaatcgattcaattgtataaatgattataatttctttcatggaagcatgatccttctgattaagaactgtaccccatattttatgctggttgtctgcaagcttgtgcgatgatgttatgttcatgttaatcctatttgtaaaatgaagtgttcctgaccttatgttaaaaagagagaagtaaataacagacattattcagttattttgtcctttatcgaaaaaccagatttcatttttcctttttgtttgtgatctcatttggaaataattggcaagttgaggtactttcttcccatgctttgtacaatataaactgttatgcctttcagtgcgttactgtgggaggagcaactaaaaaaaaaaataaagacttccaaaaatcatgaaaaataa

SEQ ID NO: 52 - DNA, 3258 bp, Mus musculus
gtctgtgcaggagtcggcagctggcgccagggcggccggaggatgcagaggggccggagccgggcgggccggaggccgagacgcgcgctgtcccccacccctatcccgtgaatccgccggccctggaacgcgctgtcgctgggcccgccgtacccgggctctcctggtgtctcttactctctactgctgagccctccccttcccgcgccgctgcgagtggccgccccaccttcgcctggttcccaagacacagtagtggaccatggaggtggcgcctgagcagcctcgctggatggcgcaccccgccgtattgaatgcgcagcaccccgactcgcaccatccgggcctggcgcataactacatggagccagcacagctgctgcctcccgacgaggtggatgtcttcttcaaccatctcgactcgcagggcaacccttactacgccaacccggcccacgcgcgcgcgcgcgtttcctacagcccggcgcatgcccgtctcaccggaggccagatgtgccgaccacacttgttgcacagcccaggcttgccgtggctggacgggggcaaagcagctctctctgccgccgctgcccatcaccacagtccctggaccgtcagcccgttctccaagaccccgctgcacccctcagctgctggagcacccggagggcctctgtctgtttacccaggggctgcgggtgggagcgggggaggcagtgggagctccgtggcctccctcacccccactgcagcccactcgggctcccatctcttcggcttcccacccacgccacccaaagaagtgtctccagaccccagcacaacaggagctgcttccccggcctcttcttctgcagggggtagtgtagcccggggtgaggacaaggatggcgtcaagtaccaagtgtcactctccgagagcatgaagatggaaggcggcagtcccctgcgcccgggcctagctaccatgggcacccagcctgcaacacaccacccgatacccacctatccctcctatgtgcccgccgcagctcatgactatggcagcagtctcttccatccaggaggcttcctgggtggccccgcctccagcttcacccctaagcagagaagcaaggctcgctcctgctcagaaggccgggagtgtgtcaactgtggtgccacagccacccctctctggcgacgagatggcacgggccactacctgtgcaatgcctgtgggctctaccacaagatgaatggacagaaccggccgctcatcaagcccaagcggaggctgtctgctgccagaagagcgggcacctgttgtgcaaattgtcagacgacaaccaccaccttatggcgccggaacgccaacggggaccctgtgtgcaacgcctgtggcctctactacaagctgcacaatgttaacaggccactgaccatgaagaaggaagggatccagacccggaatcggaagatgtccagcaaatccaagaagagcaagaaaggggctgaatgtttcgaggagctctccaagtgcatgcaagagaagtcaccgcccttcagtgcggctgccctggctggacacatggcacctgtgggacacctcccaccttttagtcactctggacacatcctacccacgcccacgcctatccacccttcctccagtctctcttttggccacccccacccgtccagcatggtgactgccatgggctaggcaagcctcccactggacagacatggacatcaagggtggtttggcagaaccagagcgaggctgggcactcccaggatgggtggaacatactcttggctcccgcccatcccaagagacccacttcctcctgccagcctagcctggccgaagccacctctccttggaggactcccagccttgtgccgccattactgtgaatatttctaactgggctgcagctcgcgtgtgcccggggtgctgcccagaaaagtgttttcacggagagtgtttgtttggagagcaaaatggacaggtttacagatttatagcaagaagagactggggatagaaaaatgaaacctttttttttctttttctttttttcttcttctgttttatttttttgatggagaaaggagtaggcaagaagaaaaataatttattttgctcttatttcttacaagaacgtgaagacatggaggcgtgtgctatttgtgttcttggggtccttctttgggacctcctgccaccagtcagggctctcgggggcagacttagaggtcctcagcctgagcctccttcaccccagcctgcctgcagggtagcccctgccctgacgcagccctagagggcagagacaattgcaggcggtcctgcgcagattcccaggccagggctgggtcacaggaaggaaacattctctggaaaggggaaacgtctcccagatcattcccctggcttccagaggccaaagctggtgtgacccaaatgggccagagctgcagcctgtgctctaggccagtcggacccctgtaaatacaaccttcttttctgctaaaccctcggccccctccccctctaagataaataagaaaatactcaaagcgaaaaccaaactgcataagcttaacccgctgatgagtggttttattttgaaactcgttttttgggtccagtcaattgtacgttgccacagaagccccgctatggaaaaaaataaataaaacctacaaaccaggcctgagcttcacagtcctttgagtggttcttgggtcccacagccctggcagggggctcgggacaagggggaatcttatgctcttggtttctgggagacagggggcaggcaggcagtggccctgtgatcccaggcttctgttctgctgtggctggctgaatccttcaaggtacagttgtacataaaaagtgtcccaagcttcgattctgtgtgtggtggtggcagtggtgcagcagccagcaagggggccccgagtgagcccagggagacgattgtgctgagtcaaccaagtgcaatatcggtgtccagttgctgcagagcaccctaaccggaagtaacttattttgtgctagtacccgcataagagaagaatcggcagtattttctgtttttatgttttgggcttgttttattttgaattagtgacctaagttattgttaactgtgtacaacatttaaatattgtctgtaaaaattgtatgctaccctcttattcctttaaagtgaatactgttaaaaataataaaatactttttgtgaaaaaaaaaaaaaaaaa

SEQ ID NO: 53 - DNA, 1823 bp, Mus musculus
cacagaaacgaaaaggagaagtgtctgtctgtgcagagactcataacgttgaccgagccgagagcagtccccagggacagtgtggaggttcgtggctctcgggcagaactcagaagagggacagctccctgacggtgtggcgtgcacgcagaaaaatgccacggtcctttctagtgaagagtaagaaggcacacacttaccaccagccccgggcacagggtgatgagctggtctggcctcctgctgtaattcctgtggcaaaagagcatagccagagtgccagccctcttctcagcacaccgcttccaagccagaccttggactggaacacaatcaaacaggagcgggagatgttgctgaaccagagccttcccaagatggcctcagccccagaggggcctctcgtgacaccccaaccccaggatggggaatcaccactctctgagtcaccccctttctacaagcccagcttctcctgggataccttggcctcctcctacagccacagctacacacagaccccctccaccatgcagtccgccttcctggagcgctccgtgaggctgtacggcagccccctcgtgcccagcacagagtctcccttggacttccgcctccgctactctccaggcatggacacttaccactgtgtcaagtgcaacaaggtgttctccacccctcatgggctagaagtgcatgtccgccgctctcacagcggaacccggccctttgcctgtgatgtctgtggcaaaacctttggccacgctgtgagcttggagcagcatactcacgtccactcacagggcgtcccagccgggtccagtcctacgcccaccttggctgtcccgggccttgaggccccacctgcacctgaccccccagggcctcgtttcctccggcaggagcgaagcttcgagtgccggatgtgtggcaaagccttcaagcgttcatccaccctgtccacccacctgctcatccactcggacactcggccctacccctgccagttctgtgggaagcgcttccaccagaagtcggacatgaagaaacacacctacatccacacaggtgagaagccccacaagtgccaggtgtgtgggaaagccttcagccagagctccaacctcatcacccacagccgcaagcacacaggcttcaagccgttcagctgtgagctgtgcaccaagggcttccagcgcaaggtggacctgcgacgtcaccgtgagagtcaacacaatctcaagtgagacggttggccgcctgctttagtgtgtctcgcctgaaggccagcctctcctttccaatcctgatcccagtcctcctggaagcagcattgcccatgagcctctctgcttcttttgagactggatgacctcaaccaagccacactcctcctctgaccacagctgatgtgggagtcagttcgggcctttctgaactgaggcagtcacagacatgctctggtctcattcaatgccagagcacagacagctacaaagccctcgtggacatggcaaaaccaggcaagagccaggatccacagagaaaccctcttgcttgcttctctgggtctagtgactgaatttttagggattctctttttggctgccacagtcagagttgtcccctggcctagaggtctggcgcccctctgcttcggccagatgtgctctctgtcccccacctctcaccacatccatccgcccactgggagtcaggctcctgaggtgagccacgaggctatgaaatcagccatgatagtagaagcctgctcctatcagttgatgtaaatgtaaataaaggtgggtttatttacatgtgaa

SEQ ID NO: 54 - DNA, 3619 bp, Mus musculus
cttgcaaagacatccccgcacagcccggccgccgcggccaccttccctgccccgagctgcagatgtggcggagcggccgggtgccgcgcggcgccaggggagcccgtgccccgtaagcctcgcgccccggcccccgcccgccccggccctccccctcgcccgctccccgctgccccaggaccgccggaacccgcagccggagccagtcagcccggcagccgaggttcctgcctcaagaccagttgggtacatctttgacacgccctcccaccatgcactccttggacgagcccctcgacctaaagctgagcatcaccaagctccgagcggcaagagagaagagagaaaggacactgggtgtggtccggcatcatgctttgcatcgagagctgggcctggtggacgatagccccgcccctggctccccaggctctccaccaccaggtttcctgctgaaccccaaattccctgagaaggtggacggacgcttttctgcagcccccttggtggacctcagcttgtcaccaccctctggactggactctcccaatggcagcagctccctgtctcctgagtgccagggcaatggggacttgcctccactgcctactgctgtggatttccagccacttcgctatttggatggtgtccccagttccttccagttcttcttgcccctgggttctggtggggctctgcacctacctgcttcctccttccttcccccccccaaggacaagtgcctctcaccagagctgcccctggccaagcagctggtgtgtcgatgggccaagtgtaaccagctctttgagctcctccaagacctggttgaccatgtcaacgaccatcatgtcaagcctgaacaggatgctcgatactgctgtcattgggagggctgcgcccgccatggccgtggcttcaatgccaggtacaagatgctcattcacatccggacacacaccaacgagaagccgcaccgatgccccacctgtaacaagagcttctcccgcctggagaacctgaagatccacaaccgctcccacacaggtgagaagccctacgtctgcccctatgagggctgcaacaagcgttactccaactccagtgaccgcttcaagcacacccgtacccactacgtagacaagccctactactgcaagatgcccggctgtcacaagcgttacacggaccccagttcactgcgcaaacacatcaaggcccatggccactttgtgtcacatgagcagcaggagctcctgcagctgcgcccaccccctaaaccaccactgcccactcctgacagtggctcctatgtcagcggggctcagatcatcatcccgaaccctgctgccctttttggaggccccagtctgcctggcctgccattacctctacctcctggcccccttgacctcagtgctctggcctgtggcaatggtggaggtgggggtgggggtattggccctgggctgccaggctctgttctgcccctcaatctggccaagaacccgctgttgccctcaccctttggggctggtggactaggcctgcctgtggtttctctcctgggtggctctgccggtagcaaggccgagggggagaagggtcgtgggtcagtacctgccagggtcctgggcctggaagatcacaagacgcccctggaaaggacggagcgcagccgctcccggccaagccctgatggactccctttgctcccaggcactgtactggacctgtccacaggcaactcagcagccagcagtccagaggtgttaactcctggctgggtggtcattccaccagggtctgtgctgctcaaaccagctgtggtaaactgaaactgggcaacccatctaccagctgtgacagcccagccacctaccgtgggctcatcccccgacgaacagaaactcttctgcgaaatagcaataatatcctactgccccagggccaagctgcagccccagacaagctgggtggcaaggtggcaaggatggtgctagaaggtctgtgctggcctcctggctccaaagtgaggacttggcttggacctgctgtccaagaagagccattctcttgggtgctaaggcctcaatcacttccatttccctagtctgtagtctgggtgaggcctttgcctgtcctcccaggacgctcagtcctgcctcttgctctggtgcgttcctctctatggccaccctggctgggcagggttccagtcctccctgccttacctacttgtcagctaggagcccccagtgatcatagataggcccacacttgggaattccttagacccctttccctctggtccacctctagagggagagcaagacagacactgctgccttctcctgcctgctgctatgacaaggtgcttctctgcttgcttagcagagaagcctgctctgccagcacttctctttcctgccagagggttggcaatactttccaaagaggaggggtagctgaccactcaaccttctccttggggatgtgcaggcaggtacaagggtcactgtgctgacctggcccggcttggcctggcctgagcagtgttactctgcagaagcttataaggcactagtctacaccacgtgggctctcaagcttgggacacagctacaggtttgggggcaccaagtaatgggatgcagggaagaagactcattggaaaggcgctttctagaaagcactgactgaagcccctgagtgttaagggaggctaggcagccaagcacagcattagcttgggaaaaagctagctagagaaaggcctggccatctggcagggttctttcctgactctagttgaacccttttcttggtgctgcctctcttgcccatgagctctgcccaagccagcgaccccagggcttgtgcaacaaaccaaggacattggaggactgtgtgctccatagctccaaaaggctagctgccccagcttgggcttcttgctgtcccctgtactatgctgcagtggctaagcccctcatgtccccatctgtagaacaagagagctgctgaggtgctagtgggtccttggctctgtggttcctctgagcagttctgtctagtctttttctcagactgcctttctgtttcagaagggtcacccttccctaacctacctgactgctggccttagaaccccagccctgatggggctcggctgtgctggctctccttgatggggctgtgagcaagttaaggagctggtctcctacctttgggtctgcctaggatccaagcaactcctggctctattagggtcctaggccttaagtccctggctgggggatttggtttgagactcaagccaaggagaagaacagagcactggacattagctcagcatgtgactcaagtgatagaccaggctcagaagggctggttacttcctgtcagtttgttgcacattccaggattcagtattttaacaggttctaagtgcctttctcttgtagtgtatggttttcctccttctggctccatggctgttagcatagagtttaaaaaaagagagataagctaatgactataacaatatattcctccatgggagaggaagtttatacagaaacaataaagtgagttgcaaagatggtttctgcacgtg

SEQ ID NO: 55 - DNA, 5665 bp, Mus musculus
tcttgtcagggcagcggcacatggactgcctgatgcgctgagcccggcgctgcggggccgcggagcgcggggagcagcggccgcggcggggaggggggtggggcgggacggcacagcctccggtgctcgcgcgaggggccggggcccgcgctttgccttctgctcatctgcagccgtctacgttttcaaaatttctctctttcctttttccccccacctccttgcaattttgaacattttgcaagactgagtgttggaggcaggtgagaaagcatcctgcactgggtcggaggagtccgcggcgctagatgcgccttccctggactgtcggcactgagaacacgaaagtttttttgtttttgtttttgtttttttttgatatttgtatgctgatgtatttacaaagacagaagtgatttttttttttcttccttgtctgcactgttttgaaagcgagtacttttggttggcaaaggacggaggaaacaaagctcatcaacatttcggggcggcgattggccttctttttaaaaagacaattgagtgcatcacgatggagaaaatgtcccgacagctccccttgaaccccacctttatcccgcctccctacggcgtgctcaggtccctgctggagaacccgctgaagctcccccttcatcctgaagacgcatttagtaaagaaaaagacaaaggaaagaagctggacgatgagagcagcagcccgaccgtcccccagtccgccttcttgggacccaccttatgggacaaaacccttccctatgacggagatactttccagctggaatacatggacttggaggaattcctgtcagagaatggcatccccccgagtccgtcgcagcacgaccacagccctcacccccctggcttgcaaccagcttcctccacggcgccctcggtcatggatctcagcagccgggccacagcgcccctccacccgggcatcccgtctccgaactgtatgcagagccccatcagaccaggccagctgttgccagcaaaccgcaatacaccgagtcccattgaccctgacaccatccaggtaccagtcgggtatgaaccagacccggcagaccttgccctctccagcatcccggggcaggaaatgtttgaccctcgcaaacggaagttctctgaggaagaactgaagccacagcccatgattaagaaagctcgcaaagtcttcattcccgatgatttgaaggatgacaagtactgggcgaggcgcagaaagaacaacatggcggccaagcgctcccgtgatgcccggcggctgaaggagaaccagatcgcaatccgggcctcattcctggagaaggagaactcggccctccgccaggaggtggctgatttaaggaaggagctgggcaaatgcaagaacatacttgccaagtacgaggccaggcacgggcccctgtaagatggcatttttgtgggctggcttttgaatagatggacagcttgtttcctgtctggtagccccacacccaaaccaacctttctgacatcagcactttaccagaggcataaacgcaactgactcatattttggtgtgcatctatgcgtatgtatgtatgtttgtgtgtgtgtatatgtgcttatggtcccatgtgtggtcagcggtgtgcatgtgtgagcgtgtatatgtgtgtattccttcacacttgtcgtttaaagatagtcctcttgttgtcttttagttccaaacaagaaaggtgccatgttttttattccaccctggagactcccatggggtttcctccccttccttctcccttcctgcccctttctgccttgctctgtgttctagcgatctcacttggccagggtctgtgttgcttcaggaagcagggccctggatctcagtgatcaagaccataagctcttgtttctgtttcagccatgaattaccatgctggaaaggtgcatagcataccctagcactgcgcgcagctgaattccttcataggttgccccacttcccttccctttttcctccccaccctttctcgttttttcctttcagttttggtgacacttgtcttcaaatttaaagtgctgtttagatttagtagagtcccatatttacttactgctacctactaagtttccttttaattccaccaaccccagataagtaagtaccattccttatagaacacagagtgtgttttgcactgtctgtacctaaagcaataatcctagtgtacgctagagcatgctgcctgagtattaactagtggacgtaggaaaaaaaattccctacctaggaatttcactgtcttttaaaaaaaaaactcaaaaatcaagtaatgcatttgtgcatgaccagaatattctcaggacagggaagcagagggcaagggaaggcctaagaatgaggggttaatttatcagtatgtatgccaaaaacataataataatgcatcttggagaagcctttgccacggtgtgcttcgtcgtgcgtctccccttcccacacccagcccattcccacttttctagcccattcactttctccatttccctcttgatattcaaaacgattgacttaagattcagtttacccactttgttttataatatatatatttgggggggggacgatattttgctgtttttagaaatcagttaattaagtgaaaaagttgatgttttgaaaagccctcccttgacccccacccccaccccccgtggtgtccttttgaatgcctcacaaatgaatgattccggagcccgtgtttctcatcctctgtttgcttttgaacatatgtgcgcctatcaagtggacttctgaaaaaaaaaatgaatgtaaaagacactggtgtatctcagaatgggatggtgttgtcacaaactgtggtttctccgatcaatttaaatgtttactatagaccaaaaggagagattattaaatcgtttaatgtttatacagagtaattataggaagttctttttttgtacagtatttttcagatataaatactgacaatgtattttggaagacatatattatatatagaaaagagaaaaaaaggaaaactattccgtgttttaaaattatatagcaaagatatatattcaccaatgctgttcagaggagtgcttgggggcttttgaaatctttaatattttaaacctattactgacacatcagcatgttttctgcttaaaattaaaattttatgacaatactgaggcttgctgtgacgactcctgctccaacgctttcttgtttcttattaggtctcagaaggaagtcagttaacgtcacccaaaagcacaaaacgggttttagtcaaatatttattggatgatccagtgttttttaggaaaagcatctgccacataaatgttcacttcaggattctgagtcgttggaatggagtatggctgccagagccccagatgattttgttcgctttcggcctgtgtatctatctcacgcagtgtaaggttgacctcggtgctatgtgtacggcttagagtttgatagctgttttgactttaaagatggctgttattttgtttcactgagttgtataatgtcaagagagtctgctgtttcttcaaaagcacatttttgtgctcgattaaaatccccttccatccatggatgttttccggtctcctgcctccacagtatctgacttctctgcggatccagtgatgccctcccccctccctcagccactccgctgtccagtgtcacagatctggaaaacgagacctgtaggtttcttcccaggttcctgttgtattccacggtgagagatgagagagtgagagaaataaaatacattttgcaagcaaattactgattaaaaaaaatgtttcaaccatgttgtatagaactgaataagagataattcagtaatagccatgggacaccaaaaagttggtccttttgaagatagagaaagagagggattgcagaactgttcaatgacagtgcttgccagggcacagatgagtacttaaggagagaagtgttcaagggttgcactgtggcccacccagacacatggaaccatccagtactcgatgaagaaaagcatgcctggcctttcttggaattgtttttaccaaataaacatcacaaatgttttctgcagaagatataaaaagatcataataaaacttattcttttgtataccaggaaaggtttgtagaaaacctacctcattcaaaacaaaacaaaacaaaaaactcaagagtttggaatctccacaggtgccttatctgtaggtaattatcagtcccccacccccaggctgacttctctttgcccctggatattaactagatgagaaaatgccattcaatgccctgagaagctagaacctagggtcagataactgcagcagaccatagccattaggaagtcccattgtcaactgtcaccatttcaaggtaggcatctttccatggttaattatttggtcattccaaatgcatctgaaatgttgcatgccctgccattcagatgcaaagcacttcctggtcaggtcatatcttttcattagtcacatggattctcgaagaaagttttaaaaaaaaaaggaatcagggtttctctctgctgatgctcccctgaggggatgggggggctttggatgacagtccccaactatagagggactggtgctttgggtcattcacaaaccacatcaggcatgacttggccaggcctttgtctctaaatagaacaggccttgaagggaccatcctttgtagaagaagaagaatgggcctatgtttgagaatagcatgatgggactcagtcactgggtgagggggaaaccagataaaagaacccagaggctctttctaacccattatcttaccgtgggaatttacactgagtctaaacataccttcgaaagggctacagcctcaggtctggatgacctgccgtgagttgtaagtggcaatgtctctcctgtccagggatgaaacaactgatgatgtgtaaaatacaaggaatactttgctctttctaaaagtttgttcagtttggcccttagcattcctttagaaaaaactcaaaacttcctcagagttaattagtgagatcctcaggcatcgatgcctgtggatctcagacagctctgtgtgtggcttgtccccaccggctgggattggcttcctgcagctgactaggctgaggtgtgatgccctccctccctgggtacataccttcttctctggctctgattgcacttgcttcctactgagggctcctgagtacaaccctctggggaagccaaattcaactccaggtgttcggtaaaaatcagtgtcaataataaatgaacaaaacttgtaggtcttgatggactgaattttcaataactactgttaatgcatttttgccaaaatctgtgttggggaatgtgtgtttgtgtgtggatgtttaagtggagacagaaaatctgtaattttttttttagcacctcaccagacataacaattaggtattatcctttcgacaacaccacaaagattgcatctgtcaaacaggctcaagttaacaagaggttagtgtaattgtataccatgatattgctggtatttatgctgtcaagtccaaaacttcatttgttcttctaaatgtttaataaactaatttttttcctttt

SEQ ID NO: 56 - DNA, 4226 bp, Mus musculus
tctgaaaacttgcagcacgggcaaaacttgggctccgggtgcagagcgcagaggccagcagcttgtccctctgcatctgtgcagtgccgccgcctgaccccgccacccgaggaggcgcggtgccacccactgctctgttccttgctagagctgagctgggcgccctaccggatcctggcagaaacttccactctctcctcggtttctgaccgcactagtcagtctctatctctgtcccgttctgtctctttgtctctgtctctctctgagtttctgtctctgtccctcttctctgtgtctctctctggctctctgcgtctctgtctctccttcccgcccccctccctctctctccctctggggtgggggagaggaggcggaattctttccccgcctaacatttcaagggacacaattcactccaagtctcttccctctccaagccgctgccgagcgtcccagtacccgcaactcccgagcctttgcggagagagcaaccctctccgcctccaactcttccctctccttcgcttcccgcctcctctccctacctccacctctacctccgccacccactgcccgcagcgcctcctcctttcctcctcctcctctcctcctcctctcttcctcctcccctctctctttttggcagccgctggacgtccggtgttgatggtggcagcggcggcagcctaagcagcagcagtagtccccggcgcactcgccagctcgcctgctctcgccgctcttgccctctccagctctctacatcccgtctcccgaaaggtgctgggcggattcggggcggcggaggccgagcggctgcagcggcggtaccggtagaggcagtggtagcggcggcgggaggcaggatgagcgcacgcggtgagggcgccgggcagccgtccacatcagcccagggacaacctgccgccccggtgccacagaagcgaggacgcggccgacccaggaagcagcagcaagagccaacctgtgagccctctcctaagagacccagaggaagacccaaaggcagcaaaaacaagagcccctctaaagcagcccagaagaaagcagagaccattggagaaaaacggccaagaggcagacctaggaaatggccacaacaagtcgttcagaagaagcctgctcaggagactgaagagacatcctcgcaagagtccgcagaggaggattagggggcgccgacattcaatttctacctcagcatcagttggatcttttgaagggagaagacactgcagtgaccagttattcttaactgccacggtctttctacttcctgcggggtggggcgggggcggggctgggcgaggggcggggccggggtgggcgaaatcgcataaccttgagaaggactatattaatcactttgtaatcccttcacagtcccaggtttagtgaaaaactgctgtaaacacgggggacacagtttaacaatgcaacttttaatgactgttttctttttccttaacttactaatagtttgtggatctgataagcaagggtgtgtggttgaagaaaacctctgtggtgggcttaatcagtcactacatgcaaaccctaaaccggcaccctggtgaccgggggcattcgtataagaaaagcattgtgtgtgactctgtgtccactcagatgccacccccaccatgatcatagaaaatctgcttaggacaccaaagatgagaactagacactactctcctttctttgtgtataatcttgtagacacttacttgatttttttttctttttttacttttcaattctgaatgagacaaaatgctggtgtatcttttcatacagctagcaaaccagaataggttatgctcgttttttgctttgttttgtttttcaaaaagggaagtaaacgagaaccgttgactcctccatttatggactcatacacagcagcaggagtgataagcccacaagctctctttcccgcctcgggaaatctacacagccaaaagccacttagccataaatgacacttgtcagccttgaagcatcggagataactagctgagtaaaatgatcctgttttggaatttaatgaaaaggttaacagtacccaatgaacccacccaagtgatgacatgggaggagcgaaaccgaaatctcttttgctatataaaggacactattttttaaaaaaaaataataaaaacagctcccgctctctgtcctctctccctcccttctctccctcgcctctctctcctctctatattccctgttcttcattgtgtaccagtgtccgtgaaagaccgcagtaccacttacctcagatgaagcctgcgtgttacatcctgtaacacctttcattttgacataagatggctagccgaggtgcattatcttggttcggactgccatctctgcattcacgctgcacttttagccagagatgcaataatccccactcctcaatactacctctgaatgctacagtgaatttacagccctgcacttgttacacgctgctagacacaagccctgcaagagaaaggaaaaaaaaaagcccaccaaaaccaaaccaaaccttactgggtcggcatctcagccatccccagttctcgaccattcttctctgtactcttactccgtctcagcaggctatgcatgtttctatgactttttttttttttaattaaaatgttacaaatgcttgtggcagctttcctgctagattgttacattaatttgaaacagttttgagtcaagttgctcctaggttcttaaggagaatttttttttcagtgacactactttgtatcacacacacacatctgtagtgttcaaatataagtctccaagtttgtacctcaaatgaattattgaaacaaatggacttcctgatttgcaaggaactacctccacacttccaaaggaacgaacttgcagcctatatcactcattgatttccttcccccatgtttgaaggagctcaaacctcacctctccctcattgaaacattttttttggtaaaagacacttgatagaaacacaatttttttacatacttttgcaaaaataaatgaattaaaatcaagccaaccttcaaagaaacttgaaattttgctacaaccagctcagccttttgcctaatgcaatgaaaaaggaaaaaaatagatttctaagatttgttgcctagaagaatatgcttgaccgatattttttcatgtattttacacaatgtgatttttgtaaaaaaatgtctcaagcagatttgttttggacgcttcttgtgtagagtttctatgcctttctctcctattaagtgtgctgactttccagagtgttacccactgggccaggaggtagtttctcatagtggcttgtgtcagtataagttaatactgaagccaaatgaaacaaacaaacaaccatgtctcttccagctgttttcagggaggttacttcaaaggccacgtgccgctctgagactggcagatggctcactgttgtgagtcgccaaaggagctatggagagattaaaattcaacatgactgttaacaatgcattaaataatcaaataaacagtggcttataaatatcagattctcattccgggtcttcggatgggccttacagaaacctcattttggccagctcataaaaactgaagcagcttctcgtgttggccagactcggcacaccgagcaatttccatctctgatgaagttattccttatttcctgtatgttgtacaatcaaaacacactactacctcttaagtcccagtatacctcatttttcatactgaaaaaaaagaaaaaaaagagaaaaaaaaaggcttgtgtccaatggaacagtgagaacatcataaaatttttatatatatagtttatttttgtgggagataaattttataagactgttttttactgttgttggtcacagctaagtaagactggacatctaacttctctaccatttctgcaagctaagtatgtttgcaggagaaacttatcaagacgattaactgcatccaactttctccccgttcctttgtgtgccctctgacttcgttctctgttccttgtgtagagtcgctgtctgtgattgtacttggacggcttgcttgtggcaataccttctctagtggattatcactgtctgcacaataaacataacagcctctgtgggcaaaaaaaaaaaaaaaaaaaaaaaaaaaaaaaaaaaaaaaaaaaaaaaaaa

SEQ ID NO: 57 - DNA, 1877 bp, Mus musculus
ctccacccaactcccccattagtgcacgagtttacctctagaggtcatcaggcaggatttacgactggacaacaaaagcacgtgattcgaagtcgtaccccatatttgggtgcctacgtaggagggaaccgagtacatgtcccagtcatttccataattcatcataaattgtgcaagggtgctatagacgcacaaacgaccgcgagccacaaatcaagcacacatatcaaaaaacaaatgagctcttattttgtaaactcattttgcggtcgctatccaaatggcccggactaccagttgcataattatggagatcatagttccgtgagcgaacaattcagggactcggcgagcatgcactccggcaggtacggctacggctacaatggcatggatctcagcgtcggccgttcgggttccggccactttggctccggcgagcgcgcccgcagctacgcggctggggccagtgcggcgcccgccgagcccaggtacagccagccggccacgtccacgcactcgccaccgcccgacccgctgccctgctcagcggtggccccctcgcccggcagcgacagccaccacggcgggaaaaactccctgggcaactccagcggcgcctcggccaacgccggcagcacccacatcagcagcagagagggggttggcacggcgtccgcagccgaggaggacgcccctgccagcagcgagcaggcgggcgcccagagcgagccgagcccggcgccgcccgctcagccccagatctacccctggatgcgcaagctgcacattagtcacgacaatataggtggcccagaaggcaaaagggcccggacggcctacactcgctaccagaccctggagctggagaaagaattccacttcaaccgctacctgacccgccgaagaaggatcgaaatagctcatgccctttgcctctccgagagacaaattaaaatctggttccaaaacaggaggatgaagtggaaaaaagataataagctgaaaagcatgagtatggccgcggcagggggggctttccgcccctgagcatctgagcggccaaagtactgagcagtagtagccgggcagctctctgtagtgtcagtactaaggtgactttctgaaactccccttgtgttccttctgtgaagaagccctgttctcgttgccctaattcatcttttaatcatgagcctgtttattgccattatagcgcctgtataagtagatctgcttctgttcatctctttgtcctgaatggctttgtcttgaaaaaaaaatagatgttttaacttatttatatgaagcaagctgtgttacttgaagtaactaaaacaaaaaaaaaaaaaaaagaaaagagaaaaaaaaactacacacacaaaaagcccccccacctcgtttagtgccaatgttgtgtgttgcacttgagttctttaatgtgcatgtacgtggaagtgttcctgtctcaatagctccaagctgttaaagatatttttattcaaactacctatattccttgtgtaattaatgctgttgtagaggtgacttgataagacacaaattaacttgttcaacgtgtagtggctagtggctctgtgacgaaaactgtgactccaagcggtgtgtccctgcgtgcctttgtaggaccctttgcacgaactctggaagtggctcttataagcgcagcttcagtgatgtatgtttttgtgaaaaagttacaaatattgtccaagtctggctgtttaagcaaactgtgatcagcttttttttttttttgtatttgtttttaaggaaaaaaaacactgactggaaacaaaacaaaataaactttctattgtaagttctcttggtctgatttatgccaaatagcaagc

SEQ ID NO: 58 - DNA, 2272 bp, Mus musculus
gaaaaaacagaagagggaaggataccagagcggttcatacagggcccagaaactaggcgaggtgacccctcagcaagacaaacacctcttgatgttgactggcgattttccccatctccagtctggggagcgggactaggcatacagatgatggagcttagaacccgctggctagggaataaaattcgctgggcagtttgtgctcaaagaagtgggccagggcgcttgtgacacaatcagggcgtttgtgacacaaacccttgagggttggcagttctctccttggcggttgctctggttgctctgtggggccttccctgtggagcaagggtgatctggccgatgtgcaagcgcctggctggctttccagtctgactagggtcggtagcccattttaggtggttgtatcatcgacggtgcgtcgcgacaggggcggtggtcactctgtttgaggtggagagagccttgtatttgacttttctaggccggccctgggggcgcgcgcgggcggggggctcacatctctgaggactgcaaggattatttacagggtattcaccaaccaaacacaacagtctaatttaacctttccaagtcctcataaatttttacagggagccacagcgaggcaaacgaatctgttggtcgctcctgactttccaccagcctgtgtggcttccgaaacaataactccttatgaaatatcataaatatagatttaaatacagtagagtgagaatgcgatttggctgcttttttatggcttcaattattgtctaattttatgtgaggggctctgctggccgtgctcacacgcgggacccgcgccttcctgatggcgtgattaattgtgatataaaatagtccgcttaagaagtgtgtgtgtctggtatgtgtgtgtgttgggggggtggcaagggagagtacagaggcaaggccagatttgatcttttaatcttcgttggccacaattaaaacaaaccagatcgtggagctgcgcgatccctttgcataaaaacatatggcttttgctataaaaattatgactgcaaaacaccgggccattaatagcgtgcggagtgatttacgcgttattgttctgccgggcggacacgtgacgcgcgtggccaatgggggcgcgggcgccggcaacttattaggtgactgtacttcacccccccctggtgccaccaagttgttacatgaaatctgcagtttcataatttcggcgggtcgggctgggccggccaggcgcgggctactgcaatggccaccaccggggccctgggcaactactatgtggactccttcctgctgggcgccgacgctgctgatgagctgggtgcgggacgctacgctccagggaccctgggtcaacccccaaggcaggcggcagctctggccgaacaccccgacttcagtccttgcagcttccagtccaaggcggcggtgtttggtgcctcgtggaacccagtgcacgcggcgggcgccaatgcggtgcctgctgcagtgtatcatcaccaccaccacccctacgtgcatccccaggcgcccgtggcggcggcggcgccggacggcaggtatatgcgctcctggctggaacccacgcccggtgcgctctccttcgcgggcttaccctccagccggccttatggcattaaacctgaaccgctctcggccagaaggggtgactgtcccacgcttgacactcacactttgtccctgactgactatgcttgtggttctcctccagttgatagagaaaaacaacccagcgaaggcgccttctccgaaaacaatgccgagaatgagagcggcggagacaagccccccatcgatcccaataacccggctgccaactggctacatgctcgctccactcggaagaagcgatgcccctacacaaaacaccagacgctggaactggagaaggagtttctgtttaacatgtacctcacacgggaccgcaggtacgaggtggcccggctgctcaacctcaccgaaaggcaggtcaagatctggttccagaaccgcaggatgaaaatgaagaaaatcaacaaggaccgagcaaaagacgagtgagccttttaggggctcatttaaaaagagagcaagctagaaagaaaaagaaaggactgtccgtctccctctgtctcctctcccccaaacccagcctccacccgcacaaaggggctctaaatcccaggcctcatctccccactggcagtccgtgctcaggctggctcttaggcctgcggctttgatggaggag

SEQ ID NO: 59 - DNA, 3390 bp, Mus musculus
aagatttctatagagcttaaagttcacagccattctgtgtagacaagagctaagaaaaatgtgagaattatacagaaaaccattaatcacttcttttctttaaatacgtatcctctctcctttgtcattattcaacagcaaatctccgcagaccggctgttgggggaaaaaagtgttagccgtctctccggatcggcaagggggaaaaattttggagccataaagttgaaaacttttttctctcagttttggaagaagccttcctttcgtcatgaacgggaccagaggagctcaggcgagagcgggcaagaggctcagaggaggggagatttctcgcctgccgctcgcgctggggctcgatgtgaatatatattatgtctgcctgttctcccctcgtcggtggctaaggtcagccgcttggaacagacccgagaggaggggggcagaaaggggaggggggtccggcgtgtcacgtgacccccaggggtgccaatgtccggtcgtgagggtatcaggcccttgcaagttgccacccactgcccgggcctcgcccagcgatgcagaaagccacctactacgacaacaccgcagctgcgctcttcggaggctactcctcgtaccctggcagcaatggtttcggctacgacgggcctccccagcccccctttcaggccgccacgcacctggagggtgactaccagcgctcagcgtgttctctgcagtccctgggcaacgccgccccacatgccaagagcaaggagctcaatggcagctgcatgaggccaggcctggccccagagcccctgcccgcacctccgggctcacccccacccagcgccgcacctaccagtaccactagcaacagcaataacgggggtgggcccagcaaaagcggcccccccaagtgcggtgccggctccaactccaccctcaccaaacagatattcccctggatgaaagagtcgaggcaaacgtccaagctgaaaaacagctcccccggcacagcagaaggttgtggtggtggcggcggcggtggtggcggcggcggcggcggtggcggcggcagcagcggcgggggcggcggcggcggcgggggaggggacaagagccccccggggtcggccgcgtccaagcgggcgcgcacggcgtacacaagcgcgcagctggtggagctggagaaggagttccacttcaaccgttatttgtgccggccgcgccgggtcgagatggccaatctgctgaacctcagcgagcgccagatcaagatctggttccagaaccgtcgcatgaagtacaagaaagaccagaaggccaaggggctggcctcgtcctctgggggtccctctccggccggaagccccccgcagcccatgcagtccacggccggcttcatgaacgccttacactccatgacccccagctacgacagcccgtccccaccagctttcggcaaaggccaccagaatgcctacgcgctgccttccaactaccagccccctcttaagggttgcggcgccccacagaagtaccccccgaccccggcgtcggagtatgagccccacgtcctccaagctaacgggggcgcctacgggacgcccaccatgcagggcagtccggtgtatgtgggcgggggtggctacgcggatccgctgccgccccctgccggcccctccctctacggcctcaatcacctttcccaccacccctcggggaacctggactacaacggggcggcccctatgggccccaaccagcatcatggaccctgtgaccctcaccccacgtacacagacctctcctctcaccacgcaccgcctcagggtagaatccaagaagcgcccaaactgacacacctgtgatgggaggggcgggctggggagggaggggtgaggttaagggccagggagacggcggacttgggggatgggcgcggtttagagtcctgaaagaggtgtgggatggggttgtcgccaggtttccagaaacagaacaggcctggggtgccttcccagctcccaggccccctccccgcccccaggggctgtcttttaagtcctccatgcctggttccatctgtctgccaacaacccttggaaaggaatccacagcaggttggagacgggaccacatcaatcccagggttcccagggttccagcactaccaagtgggaatttcagattggagaggtgacgtggagtcgggtagagggagccaagacaggcaagcagggcagagaagcacattcaacacttaatggcttggctatcagcaagcaatcaacttaccttgcatctctgtgggtaatttccccaaaatcttgattttttttgttctccctttcagtctccctgtgaaaaataagaaaatacatttaaatcgaagggcacaaagtatgtttctctcctacttccctgaagcttcaactccccccatcccccatctgttaggcttcttactatatctatcatttactatggccctacggttggttttctgctctaaggacattcatatctacaccccacccctattgattacaattttcagagggctcagggatggtaagatatcctgaaagtcagagctgcctataatataaataaatatatattttttttcttttaaagaaaagttaagaggctaaggcaggcaagttgtcaggactggaggcttgcccattctccagtctctcagctcccaccccatcccactccctccactttctttttacttctgttcttctcttgacttaacgtgaaaacagggtatatttgaacaaactgtctgtcctggcaggtgctgcagctggacctgtggaccttgctccagttcctgacaggacagtcttgttgcacttggagtttacattttaatggatattaaaaaaaaaaaaaacaactgtgagagatgtctgggcctgcagaaagtccagcttcgctcaaaaagcgtgtgttctagtgaacattttcatatatatttattggttatagcctgttaaaatattttcttttttgtattatttatccccctacattatgtatttatatgagggacaaaaaaaaaaaagaaaaatgtacttttttagtatttacttgttataaaggttgttgtgtttcctgtcatgtaaaaccagctattttagtttttattgtattctagacaagagctgtagatttatgttaaactcgtacatatgaggaattgtaattagttctaaaaggcatgaactcagctcctaaatgtcactgtatagtcccgaatttgtagagataagaagttaattctctcttggaactttctttgttcttccgtagttatttttttcccccttagttaaaagagttgtctgtcaaaacaattcttgaataaactttctgttattaatttta

SEQ ID NO: 60 - DNA, 2566 bp, Mus musculus
agctccctcttatcctggggaggagatggggccctatgggagtcagagaaaggttggtggagattaggtagggcaggggtgggggctacacccgcagccccaacttctgagggatgcagggtttgtgctctgcccagccccctcacccccaaacaggccctccaattaacctgctcactctttcttagttcatcacccctctctgttgtggagaaagccaggcgaggagggggtctccccgcgggagccctatgtaaatcctggtgttgggtgggtggggaggggtagagaaggggaaataaacctctttggctggagtagggtccgggtgagcagatttccttatccgggaatcgcaggccgggtggccattggctcggaggatcacgtgggcctctaactttgttcacttgacagtaagtaggagggctttcggaaacaggaaaacgagtcaggggtcggaataaattttagtatattttgtgggcaattcccagaaattaatggctatgagttcctttttgatcaactcaaactatgtcgaccccaagttccctccgtgcgaggagtattcacagagcgattacctacccagcgaccactcgcccgggtactacgccggcggccagaggcgagagagcggcttccagccggaggcggcctttgggcgccgggcgccgtgcactgtgcagcgctacgcggcctgccgagaccccgggcccccgccacctccgccgcccccgccgcccccgccaccgcccgggctgtcccctcgggctccagtgcagccaacagccggggccctcctcccggagcccgggcagcgcagcgaggcggtcagcagcagccccccgccgcctccctgcgcccagaaccccctgcatcccagcccgtcccactccgcgtgcaaagagcccgtcgtctacccctggatgcgcaaagttcacgtgagcacggtaaaccccaattacgccggcggggagcccaagcgctctcggaccgcctacactcgccagcaggtcctggagttggagaaggagtttcactacaatcgctacctgacgcgccgccggagggtggagatcgcccacgcgctctgcctgtccgagcgccagatcaagatctggttccagaatcggcgcatgaagtggaaaaaagaccacaagttgcccaacaccaagatccgctcgggtggcaccgcgggcgcagccggaggcccccctggccggcccaacggaggcccccctgcgctctagtgccccccaagcaggagttcgaacatgggggggtggggggaacagcgagcaccgaagggggtgcggggtatgggagggtccccgggcttgagcccagaaaaaatctatctaccctaccctcactttatctataaggaataaacacagagaaggggggtagggaagccttatttatagaaaggacaataagggagccgggtaaagtccttcggagacaagattcgagtctcttgctttcttcctttaaaaaaaaaaaagaaagaaagaaagaaggaaagaaagagagagagagaaaagaaaaagaagaaaggaaggaagcaagaaaaggaggaagaaaggaaaagacagaagagaaatggaggagtctgctgcgcctggttttcagctttggtgaagatggatccaggcttcatctttaatcacgccaggcccgggcccatctgtcttgtttactctgccgaggagaagacgggcgagcgagcttcggtggcgaccattacctcgacacttggctaacaaatgaggccaggctcggccgctgccgccgcctctgctgctgtcgctgctggatcacagcctggatttttctttctttgtcccctactcctgacacccagcgaatgcaccctcagactgccagatagcacagtgttttggccacggtaacaaacacacacacatataactttcctccctgtctgtacccactttggggtgggggtgggtgggaagactgctcactcccttccaccatagacttagaagggggaacagaagggaattgaagggcagtctgcacaacgtgggttcccaaatccgagcccaagaaataaatgaaaatgaaaaaagaaaggtagcaattgggacacccaagaaggccttctgctagaaggtccagctaggcctggcagggtgaggggcagttgagttctgggagctgggaatgtcttctgggcagttcacagtagtagagtcaaggccttctcttaggttacaaatgaatgtgaaattaggaaataaaatactgtggccctcctactctggaaggacaatgttgcagaaccctctcccgttgttatcattgttgcatcgtttattattattattattattattattattattattattattattattattattttatgtcatgtgtgtcctctctcctgttctctttctgacattccaaaaccaggccccttcctacctctggggctgcctgagcctagaaccttttgttggtgtgaaaatttgtgtcctgtacagagtgacaacagaaataaatgtttggtttcttgtgaccatca

SEQ ID NO: 61 - DNA, 1912 bp, Mus musculus
gaagtacagtgcatcgctataattcattaatacatcataaatcgtgaagcacagggttataacgaccacgatccacaaatcaagccctccaaaatcacccaaatgagctcgtactttgtaaactccttctcggggcgttatccaaatggcccggactatcagttgctaaattatggcagtggcagctctctgagcggctcttacagggatcccgctgccatgcacaccggctcttacggctacaattacaatgggatggatctcagcgtcaaccgctcctcggcctcctccagccactttggggcggtgggcgagagctcgcgcgccttccccgcgtccgcccaggaaccccgcttcaggcaggcgacgtccagctgctccctgtcctcgcccgagtccctgccctgcactaacggcgacagccacggcgccaagccctctgcttcgtccccttccgaccaggcgaccccagccagctccagcgccaatttcaccgaaatagacgaggccagcgcgtcctctgagcccgaggaagcggcgagccagctaagcagccccagcttggctcgagcacagccagagcccatggccacctctacggccgcgcccgaggggcagactccacagatattcccctggatgaggaagcttcacatcagccacgatatgactgggccagacggaaaaagggcccggaccgcctatactcgctaccagaccctggagctggaaaaggaattccacttcaatcgctacctgacccggcggcgacgtatcgagatcgcccacgcgctttgcctgtccgagcgtcagatcaaaatctggttccagaaccgtcgcatgaagtggaagaaagacaacaaactgaaaagtatgagtctggctacagccggcagcgccttccaaccttgagcccatccggaggagccctgggcggcccgagagcccgcaccaaccccagctcgacccttccaatcttccctgcactgccgctgcccgctggggaccagttcccacgagcctgtcacaccccagtcctgtgttacaatttttcgtttggtcttaggtcttcccatggctccctctctcctggactggttatcttgttattattgttaataataattattattattatttccccctccgtgctccccacttctcttggctcgcccccccccaagttgccagtgtttctgaatgtcctcgtgtctgtggttgcgctcctttccccaggaaaaagaaaagaaaaagaaattcgcatgtttaatgtgacttcccctccccgtctgtgttctaacttatttataaaaggatgatggctgtattttgagtttctgctggaaacttccataaggggcagcagttgaggttgggtagtgctgggcccagctgagctggcctgggaaatggagcccactgtctgtgtctcttttctcccacctcatccttcttcagccccacccagcccccactccctaggctcaggtagcttgttccttggggtgggaagggagctagggaagggtcaaagtgtggacattgagaagaggagaggaaaggagcaagagctgaactcctgctgcctggtaggccccacaaggcctagtctggaagcgtatggaatcagaaataatcctcagtgtaaaatgtcttgtgatttttctctgtgaatccgtgggtctggctagaaggcccaatgctgtaaatatggggatagtctgggtcaggccaatcacttcctctctcacccatttcgcttccaagaccatttgtagtgagcgggtggatgctgtgctacgtgtgaaatctgtctttgccaggcctgtctcagtgattagcttttggtatgtctgtagctttccttgaagttgaataaatgtttcccccactccaaaaaaaaaaaaaaaaa

SEQ ID NO: 62 - DNA, 3902 bp, Mus musculus
ggacgggcagggcgcgcactctcggcggtgcacacgccgctccaactctcaggcgcgcggggtgcgtccgcccgccgccgccgccccggcaccgggaggcagagcaagcgctttgtccgcacgcccgctcgggactctgcccggaggaggcagccgcgccgagtccccgcctccgcctctgcccccgggcgggccgggccggccgcggtggggggagccaggctgagggcgagggtgggggcgggggcgggcgcaggaggagagaggagggcagcggaggcggcgggagcgccgggtgccgggccgggggagccgcgggctctcgggaagacgcggatgatgaacaagctgtacattgggaacctgagccccgccgtcaccgccgacgacctccggcagctcttcggggacaggaagctgcccctggcgggacaggtcctactcaagtccggctacgccttcgtggactaccccgaccagaactgggccatccgcgccatcgagaccctctcgggtaaagtggaattgcatgggaaaatcatggaagttgactactcagtctctaaaaagctaaggagcagaagaatccagattcggaacatcccgcctcacctgcagtgggaggtgttggatgggctgttggctgaatatgggacagtggagaacgtggagcaagtcaacacagatacagaaactgccgttgtcaacgtcacctatatgacaagagaagaggcaaagctagctattgagaagctcagtgggcatcagtttgaggactactccttcaagatttcctacatccccgatgaagaggtgagctctccttcaccccctcatcgtgcccgggaacaaggccacggccccgggagctcttctcaggccagacagattgatttcccgctgcggatcctggtccccacccagtttgttggtgccatcatcggaaaggagggcttgaccataaagaacatcactaagcagacccagtcccgggtagacatccacagaaaggagaactctggggctgcagagaagcctgtcacaatccatgctaccccagaagggacatctgaagcatgccgcatgattcttgagattatgcaaaaagaagctgatgagaccaaactggctgaggaggttcctctgaaaatcctggcccacaatggcttcgttggaagactgattggcaaagaaggcagaaacctgaagaaaatagaacatgagacagggaccaagataaccatctcatccttgcaggatttgagcatttataaccccgagagaaccatcaccgtgaggggcaccattgaagcctgtgccaatgctgagatagagattatgaagaagctccgagaggcctttgagaacgacatgctggccgttaaccaacaagccaatctgatcccagggctaaacctcagtgcacttggcatcttttcgactggactgtctgtgcttcctccaccagcagggccccgtggagttccccccagtcctccctatcacccctttgctacccactccggatacttctccagtctgtaccctcatcaccatttcggcccattcccacatcatcactcctacccagagcaggagactgtaagtctcttcatcccaacccaggctgtgggtgctatcatcgggaagaagggggcacacatcaaacagctcgctcgatttgctggtgcctccatcaagattgctccagcagaaggtccagatgtcagtgagaggatggtcatcatcactggtcctcctgaagcccagtttaaggctcagggacggatctttgggaaactgaaggaagaaaacttctttaatcccaaagaagaagtgaagctggaggcccacatccgagtcccctcgtcgaccgctggccgggtgattggcaagggcgggaaaaccgtgaacgagctgcagaacttgacaagtgcagaagttatcgtgcctcgtgaccaaacgccagacgagaatgaggaagtgatcgtcagaattatcgggcatttttttgctagccagactgcacaacgcaagatcagggaaattgtacagcaggtgaagcagcaggagcagagataccctcagggagtcgccccacagcgcagcaagtgaggctcccacagcaccagcaagcaaccgatgaatgtagccctcccaacacctgacagatgagaccaaacagccagcagatcgggagcaaaccaaagagcatcccgaggagtgcgcagtctgcagagcagccagggcctgcagacctctacacatcctgggatccaggagggcacagggaaggccaggttgtccagaaacaccgcttggcctgcccccagcttcccctggcttctgcaggcataacagccatccactgcccatccaactcagattctcctcagttcccaggacgctatccctttcggttgaactaacataggtgaacatgctcaaagcaaagcaaaattcctagccgtttctttgttgtggaaagttgtctctgtacatgtatgtacatatcagaaggggaaggatgttaagaaatgtggcctgtgggttacacagggtgcctgcagcgttaatatattttagaaataatatatcaaataactccactaacaacaatttttactcagtgattaattttgttttctttttgaagagaaagcaggcttttctagacttccaagaataaagtattgggtgaggtccactggtatagaaaggaactttgagccacccacacaaaaaattcacttggagggaaatcttgtcaaggacacttaatggcagttgtgggtcacctgtgtctgtcaacagaagggataccgtccctgaggaggggccctaggccctggtcacctgtttctcccacacacccattgctcagtttcacaggtttttaaactgatgatttttggtttgttttgtttttgttttttgcatactgctatatagttctgtctctctccttatctccctcctaaccttcctctgtcttctttacccaaaattcttttgagtcccttcgttctttttctgctccccgtatctatgcacccctcccctccccccaggcaaagcagtgctctgaatatcataccacacacaaggaacaaatgcgaaccacacagaccagcctcagcttacactcggttactcaagcgaacaagagtcggcggtacttgtcctagcattcggaagaggaaagcaggatcccagcaaataaaccagccagccaaacaaagaaaaatcccacaaagaaagaatggactctctctccatcttggtatacaagccatcaacatttagtgaaattctttctttctttaaaaatttaaagtgcaggaaaatagcaatttatacgaggttgttggcccagggcgttaaatttacagattttttttaaatgagaaaaacacacaaataaaagctacctcaggtgttttttacctcagcaccttgctcttgtgtttcccttagagattttgtaaaactgatagttggagcattttttatttttttaataaaaatgagttgaaaaaaaaaaaagatgagctgccagccccaagcaggcggcagcccaggtgtgccgcagctcttctgtcttccgctagccaagagcctacgtggccttcttgtggacaaacctaaaaatgtttattttttaaaaagtgacaaagaaaaacagagaatattggagccgtcctgaatttcaatagggtacgtgccgtgagggcttttgcgctgaaggatgaccacatcctggttatgtgaagaaaccatgacactaccagactgcgatgccggtttcctctactgcaagcagtgttctgtgacaaaaaaaaaaaaaaaaaaaaaaaaaaaaaaaaaaaaaggaaaaaaatatatccaagctaacaagaaaa

SEQ ID NO: 63 - DNA, 9457 bp, Mus musculus
tgtttgctggattcggtcttggaatgacccacctgtaaagtgctttccctcctcctcggcttcaactctgcagggggctttgcctggagggggcagggagggaaggggggggggacacaaaaaacttctttctttctttctttctttctccgtttctcttcagcccgacattgtcacctcctccttgaggggttagaagaagctgggagctcccgacagagctggaaatggtgatgactgttttttaatcagaggacaatttcttttcactgcactttgactatggaaacagacgcaattgatggctatataacatgtgacaatgagctttcacccgaaggggaacacgccaatatggccattgacctcacctcaagcacacccaatggacagcacgcctcgccaagtcacatgacaagcacaaattctgtaaagctggaaatgcagagtgatgaagagtgtgacaggcagcccctgagccgtgaggatgagatcaggggccacgatgaggggagcagcctagaagaacccctaattgagagcagcgaggtggccgacaacaggaaagtccaggaccttcaaggcgagggaggaatccggcttccgaatggtaaactgaaatgtgacgtctgtggcatggtttgcattgggcccaatgtgcttatggtacataaaaggagtcacactggtgagcggcccttccactgtaaccagtgcggagcttcttttacccagaagggcaaccttctgagacacataaagttacactctggagagaagcccttcaaatgtcctttctgtagctatgcttgtagaagaagggacgctctcacaggacacctcaggacccattctgtgggtaaacctcacaagtgtaactactgtggccgaagctacaagcagcgcagctcactggaggaacacaaggaacgctgtcacaactatctccagaatgtcagcatggaggctgccgggcaggtcatgagtcaccatgtaccgcctatggaagattgtaaggaacaagagcctatcatggacaacaatatttctctggtgccttttgagagacctgctgtcatagagaagctcacggcaaatatgggaaagcgcaaaagctccactcctcagaagtttgtgggggaaaagcttatgcgattcagctacccagatattcattttgatatgaacttaacatatgagaaggaggctgagctgatgcagtctcatatgatggaccaagccatcaacaatgcaatcacctaccttggagctgaggcccttcaccctctgatgcagcatgcaccaagcacaatcgctgaggtggccccagttataagctcagcttattctcaggtctatcatccaaacaggatagaaagacccattagcagggaaacatctgatagtcacgaaaacaacatggatggccccatctctctcatcagaccaaagagtcgaccccaggaaagagaggcctcgcccagcaatagctgcctcgattctactgactcagaaagtagccatgatgaccgccagtcctaccaaggaaaccctgccttaaatcccaagaggaaacaaagcccagcttacatgaaggaggatgtcaaggctttggatgctaccaaggcccccaagggctctctgaaggacatctataaggttttcaatggagaaggagaacagataagggccttcaagtgtgagcactgccgagtcctttttctagaccatgtcatgtacaccattcacatgggttgccatggctaccgggacccactggaatgcaacatctgtggctacagaagccaggaccgctacgaattttcatcacacattgttcgaggggagcacacattccactaggccttttcattccaaaggggacccctatgaagaactgcacatgaagaaatactgcacttacagtcccaccttccctcggatggcgacatgctgtcttctggatgctgtcactgtctataattcttattttgtggacaaaatgtcatttgctctgcctaactacaatgaggaagaaacaaaagaaaagggatgggatgttcaatgataacttggcttgtttatttcgtgagcatttaaagcagttcattgcagccatgcatccttgttaaggcctatcataatttaggagatcattcagttcatagaggttcatccaagagattctgatctgccattcatattcaggattgtgatagaaggcaggaaagttgagagttttctgggtaggatgcttggcaatttaaaatggtctaagtcattttactctcaaagaagtttcaaaatgtaaaccgattttattttctgttcgttagagatcatggaacacaaaaacaatgttattttccataactactaggatgagttgaattgttgtgggttctgtgtttacctcccctacggaatttataattgagtatgttttacactgtatcatatagcaaaaattttaaactacaggtagtcaagggccgctacgatacatctgaggtcctttgatcttatttttctaaacttggcgcactgtttttccatagttttgatgactggcattttatagacaccctggcagccttacttttaacacctgtaacaaatagtattttttatgtagttttcagaataacatatggtctcaagagtgggtaagaggcagtcagtaatttccaggaagaattctgcttttcacaatttgagatttttttttaagctgtaatatgatggcagcttagtatacatgtttgcttctaaaggtgtgtttacgatcgtcactttatcagcattcaatcagtgttaacaagtcagcagaaaagtgtaattaggcgaacagtagggggaattcccactctaagaacccttttctggtatttctcttcaagctgtgaccactcagtgttctgtccacagtccattcctctttgactcttggaatagaaggtcttaaaaagccttgctagcagttgcttcttttaaaatcttggagcaactgttaattggacctgaaagtggtaacttgaaccctggaaggtcttttatttactagggctatttattccactatttcttcagtaataagagttactacctttgctggagacaaaagggaacttctttgttacctgaataacatagtgcaggtcaaacaaacaagcaaaaagttttctttcagccttcccggagttgagaatgtgaccatctttaggagaaggtcccgcactttaaaaataaaaacaaaaaatccaactctagagttccttatcagaaatgaatctcaaccccttagtcaagaattttctgtcgtctcttaatttgcacgtgagaaatacttccatatctcatgctcaggttttaatctgatctttgtgagttctcaattgtttttttttttttttttctttttcttttctgaacttgacttgcatttgtgggtaggattggagcacactgttatcaaggcattcgggggaatcaacttggcagacataacttgctgcttgtgtttgaggaaaacaaacctgaccataactggattatttgacagtcatttgtaaatgctgagaggtctgctgtgtggtaacacatcgctgctccaacagtcccttgagtgacatgcccaaaccatgtcactgcactttgccttgggaagtaccacgactggagaagtacccacggatgagcagtgtgctccgaactcgctttgcaggtttctgtctgatcatttaaggaacttcttagctcttccatagccagagcgaggtagttctgacttgcctcttttactaatccttatcttagttctttctggagaaatatccaactggttccttaacatgtaagagaatgcaccaaaccagagctatatcaaatgccaattgttggttttgtttttttttttttggcgggggggggggggggggttgtatcccttggtacattttaaaaacatagcttcctcggggatgaaacatctcatcatgccttccctatccccacttaccctacacactctggaatcacagtgaaaaggatatttatttctgaatgaggtaaataagtttaaggctcctgacttctttgttttaactgcaacttgaggaagcatctggatgtctgtcagtgtgtccctttcggctctttgtgaacacctgcaggcatctgctatctcttccttctctctgctagcacctgctctattaacaatcatgtacctacctgccctgggatgacactgaatttaggtgtactggaagagttgtgtccttcccctacgaaggtggtaccgatgtgctctgcctttctgatcccattttattggaaaacctgatgtactcaaaggcgtgaactataagggtagattggctgtacatatgcacttagccttttctttctttttcagctgcatggcacaagatacagaaaaaaaagtagatagctctgctgtgacagggacaaatggggacaagctgaattcaagatttgtgggttacgataaactttgtggacatagtgctgtctgagggaccctcaatgactgtatttgcctctttttcaccaaagagaataggagacatggtagcacctaaaaataggtatgaaggccagataccaaataaatcaaatttacataaccgttacaagaccagtttatttaggtgacattttacagttgagagagaatttgaggggcacgaaaatgggttatcctctgtgttttccagtttgtcaggaatttatcagatggctctgccctaattttgcccagatttttttttttttatcaccagttccttctggagaaacatgctgcgtctctatagtagtgattgattgcttgttataaaggctttttagaacagtgagagccgccagcccaatgagcggtagagggaagtgtgaaacactgtcattcaaacattccgttaataattctctggataaaatggggatgtgagggccaaccattaagatttagaatgaagttggctgcctggctggcatacatacaggtcagtgtttgaatgcagtacagtttgcatgcacatgcatattgcacattcttttaatgaaaattcacagaaaagaagcaattagcattgccagttgtattacaaaagcttagagttagaagtacagtacagttactacgccagcagttgggtctaattcagcaggttgtattaagaaagccccatgttgtcagtgtgtctttaaggctaatggcaatttaaatcacaaatctccattgtctgcatccagaaaaacaaacaaagcaaggcaaaaaacaatgaaacaatgaaaagatattgagttgactggagaatatttgaagaaatatagattttttttgtgagaggaagaagttgcaggaagtaatttagcaaccttggcttgtgtttgtgcttctctgacccagttgcacacctcacaagtttggtctttccaaaccttcatcttctctcctaaatgacaagttagggtagaaagattcatggaagaggctggagaggacaaggagagctgtctgcccctagattcaggactgcgccttacactgtccaagtcacctgccacatactgtcttagccttgaaattgtagattggtggtgatcgtcagttgtacttcatgttattttaataactactgtctgtataattattctacagcaaaagctcccccaaatccaaaaggggaaaaaagtgagggatgaattgaaactttgtatatataaaagttattttataaatgttttagtcttccagtttctgcaaaataattaaaatatacagtaactggtctcttaaaccctgaacttaatgtattaaatacttataaaattttatattggtgccttttaaaaatgcattgagagtgttggttagctctttagctctaccacacttttactgtgtattttttaagaaaaaaaaaaatcacataaaatctaagtactctctaattcacctttgtgttcttagaagaagaagaaaaaaactgctaaaagaatttccagacttttagctgagctggcaaaagtgacaaaaaaaaaaaaaaaaaaaacaaactttgaaaccagattttaatctttttagcttgatgatatttctggacattattctatggtagaggcaaaatagaaggtagactcacatatcagcaatgaattggattatgctatatgttttgatttcttcttgaggaagaatatgttttgcatcacacaggggtttctttgtataatttggagtcagtaattacagtattgctttgctttgttttaaatacccgactgatgttactctagtcttgggtcttctgtctcttctgtcttctaacctgtggttgttttgtgaaatggcgtgggaatgcagatcattgtctcctcttgaacaagacaaagctcagcacactagtcccgtgactggtctccaaaatgtcatctcagatgtgtagtctggggtcgtctgtcacttctaaggggtgtcttcaactatgacagctttttaaaaacgagctgctggctgggtaaagcagacatcatatgaccaagaagctgtgatacgctagtgtttccttctgtcatcgtgttgtactcttaggccaaataatgacaccttgactgtttttacatttactacagaaacctaaacttttggaattttcaaaggtttttatgtaatgttctatttcacagcggtttccttttctcttgctgtactgagtctgtgagtattttttttttcagatgcactcttggaaataagttctgaattctgtttatggcgtttctctcgcttgacactccgtgtatagatgttctgtataagtttgttatgaatccacctttctcatgtatggacagtgagaagtacatgaacaactagtttcctaagatgacttttatattatgaattaatatgaaatttacaggtcatgtgcaccttaagtgcagaatcaaaacttcctataggtccacataagcagttctcagtgaaccgggccttcagtttacctcaagtgacatgtagtaaggaaagcccgggaacatggaggtcactctctgcagcacagtgcacttagtagcatgttgctgaatctcagttacagaaaatggatatttaaaggaacacactggaaggatgcaagggattgagggactgctgatattttcgttgttgctgttgctttgtgcttttcaaaaagttgttcaaattatggaactgtgatctaaaaacggtttgacagtggagcaccagagaggatgtccaccactcacttgcccttcggtacgactaagctctgagtttgcgtcttcaactctaccttcctctcccctcctcatccctactctgtctgtggccctcctctcggtgtcatttcctcttggtggctctcttgacactcctactagttaggaggtcaactctgaacttctgactcattaaatcttaatttttgtctcagtggttttagttagctgtgtagtctgctggcgtatttttatacccacgtgtagttttatatttttaaagcgtggttgagttgacatggtgcactccgtagtccttattttgcttcactctcaagtaggaggcacctaacgtgactccagaaattctgtaatcagagtatccgtctggctcctttgtgcttggattttgatttgtttggctttcttcttaaatacaaatcatacaagagtagactttccccccaccatagaggataaaaatgtaaagcaaagagtcttaactttctcccaaccggggggcggggcgagtgctgtcccacttctgcagtgatgttccaggctcagaggggtctccagaccccaggcttgcctgtcatcgggtctcttcgaccctgttatttgcagctttgggatggtttcttttgctatggagtttaacagctcttcttttttccataaaagtgcaatacccctcttgaagtcttaaaaactatggtaattttcttcttttctttctctgacatttgtctcattagccaatgatgaaaagaaatctttggaaattctaacatgtcataggacataggcaaatatggaaaccactctgctatgaaaagttgaaaaaaaaaacgtattaacaaacgactcctctgactttctagaaacttcgtttagactaaattttctttgaaatcatgaatgaaagtttatcctgaggctgaaatataatttttctcccactcttgtctaatgctcttaagttaaataactaatatttaatgttttcatttctctcattttggaaaataaatatcaaagcacttaggtcaggtttaaatcaatattacctgttggaaaaaaatttttttacatatttaaagcagacctgtttcaatgcagatggagaagtatcttttgtattattcatatttatatgtagtgtgttcttttctttttttaatacctgtgtttctgtagtaagactgctgtaatgtaaatacacgttttattaaaaagtaacatttctttggcatttcttttgatggcacatactgtatttttacagtatatgtcttggctacttaacttttttttctttaataataccaaaggtaattagactattttaaggactaattgcttgacagtttctagactactttgatttttttagaagaaaaaagaaaaaaggataaaaaaaagtcaaaccagtgaacctcattttttcaaactaataatttggggaaataaaaactattgtttaaaaagaaatatatataaatatatatataaatatctgtaaaattaaaatcccagaccttgtatgtcaggtttgctcagtgtaatgtagggtttttttttgttttgtttttgtttttgtttttaaaggctcaaatacctcagaaaatggggtttactatggaaatactgcgacagtctctgcagctgtgtgagctgtcattctgctgcatactgattgggagacctccactaaacagttttatcactgcagactaaaatgtgggacttgtattttctttgtttttaatgcacacacgtgcatgctctgtgcgtgtatgtgggtaccgtgtatatgtgtgtgagtgttgtatatgcatgtgtgaatgtgtgtgtgtgtgcgtgcttgtgtgtgtgtgtgcgtgcttgtgtgtgtgtgtgtgtgtgtgtgtgtgtgtacaactgaagaagctgcaaaaactttgtaatactttgtgaaagggttatattataaaggtttgtactgtctgagtgcacagctactggaataaacttagccaatctcaggaacaagcatataatttgtccaagatttatttcttctcagaagtgtaagtgcagtttttaattctgtatattatttaatattttaccaataaaataaacttctgacagaaattatttggtacaaaaaaaaaaaaaaaaaaaaaaaaaaaaaaaaaaaaaa

SEQ ID NO: 64 - DNA, 5742 bp, Mus musculus
ggatcaatgtgactctagaaacaaatggatgaatgaatatccatatgaagaggaaaaccataaagaatctcagcgcccttgagaacagaatgctaatgcttgatggaatgccggcagtcagagtcaaaaccgagcttgtggaatctgaacaagggtctccaaacgtccacaactaccccgatatggaagctgtccccctcttgctaaataatgtgaaaggggagcccccagaggactcactgcctgtagatcactttcagacacaaactgagccagtggacttgtcaatcaacaaagccaggacctcccctactgcagcgtcatcctcccctgtctccatgacagcgtccgcctcctcaccttcttcaacttcaacctcttcatcctcttctagtcgtccagcctcatcccccactgttataacatcagtatcttcagcatcgtcttcgtcaacagtgttatctccaggaccccttgttgcctctgcatctggcgtgggcggccagcagttcttgcacatcatccatccggtcccaccttccagtcccatgaacttacagtctaacaaactcagtcatgttcatcgaatccctgtggtggtacagtcagtgccggttgtctacacagctgtacggtcacctggaaatgtgaacaacactattgttgtaccgctcctggaggatgggaggagccatggcaaagcacaaatggagccccgaggcctatctcccagacagagtaaaagtgacagtgacgatgatgacctgccaaatgtgaccttagatagcgttaatgaaactggatccacggccctttccatagccagagcagtacaagaggtacatccatccccggtatccagggtccgaggaaatcgaatgaataaccagaagtttgcttgttcaatctcaccatttagcattgagagcacaagacgccagagacgatcagaatccccagactccaggaaacggcgcatccacagatgtgacttcgaagggtgcaacaaagtctatacaaaaagttctcacctgaaggcacaccggagaacacacacaggagagaagccctacaaatgcacgtgggaaggctgcacctggaagtttgcccgttcagatgaactgaccagacactaccgcaaacacacgggagtgaagccattcaagtgcgcggactgtgaccgcagcttctcgaggtcagaccacctggcactgcaccgtaggaggcacatgctggtgtgaggaaggctccggtccagctacgtgcaagagctgggtctcttcaggagcaactaactcagcagggctgagctccctctacagtgttaacgtcaagggcaccgccattcccacggtgcctgaaaccagagcaggaacaagaaggaacccttctcccttcggcctgaaggtaaccccccatcatgaccacacgcgtgagagagccgtctgcgttcacgctggcctgggagttcagcgcctcaggtcctgaagagacaggcattgttttccatgctatgtcctctgccattgaaaagatgtttgtagcttgtacattttctgagctggcagacactctgctatcagtccctttaaaggtcatctaccgcaaattgatggttagaggagaccttctcagtttggatgattcttcctttccaccctcaccctaaccctagccccttttgacaacgatttcagttcctatctcagtaagctagaacacacacccagtctgttaccagcaagcagcatggactagaaaggaaggggctattggaggcactctgttacccaaaaacaaaggcacacagaagtagccctctgtgctagatagcgctcataccttgtcctcatgccatcccccaaaagaaccaacattgagtcttttcatttgtttgttgttgcttgtttctgttctgtttctattctgtttttgtgcatcagttccagcagaggggcagagcagtttctcaaagactagtcctgctccgccctggcatggactgacagagtattccgtagttgaataggaagagcctgtctaaataataataacaataataataactactgtcccacttcaaattgcggtgttctgtcacctaggcaccatctcctcctgcccttagtgtttgattacaaagaatcgaggagaggggggctttcttagacacactggcaccacggtaagaggtggagctgcccaggcaaagtcactgaacatgaaaacgagacaaagcagagatggaaataatgctcttcttgaggagaaaagcaataatgaatagaaggactttcctacaataactccactgaggactcacattaccgattttcatacttactaaagggattgtaaaaaacaccccagcatctcagaggtcttagttacagccacagcacggaggtaatctttccactttctgttgtcctacttgcttaagtaccacacttagagattatgctccccacttcttggtaggaaacagttacttggtgactagagaggcacaggaggcagagctgccgatgaactcttccgcctgtgggtctcttctgcttgggcctgagtccgtggcaagagtgtccccatgggaagcctggcaagagtgtccccatgggaagcctgccttggtgctacttctccaacctggagagccgagagtctgactctacgccattcactttttaaactgtcccagaatccccgtgctcccacattggcatccacagggacagaccacctgcttcttctgggagaagggtcagagcaaagctaggttccatcctatctctcaagtaaagagctttttgtatttagcgaaatgtacaggaaaagcaatatatccccccaaccccccaacaagcactctttccaaaatagtctgtcaccactgaaagctgctgttttcccagctttctgtggcaaattgggtttggtgagatttttgttgttaagacattttatagcttaaatattaaaggtctccagtgaaggcctttgcaatatatttcaatgacacacttgatttgggaaactgcacaaatcaacctcatagggttcctagctcagttttgaatgtctgcacattctaaaatgatttctggtgacagttatccttcctagtatcagcttctaatcaggataattgtattctgacaaagcaagagctatgaacattacaaggagaaccatttgtgtcgggtagctttgaaaaagacttttaataactgttcaggcaaaagtagcatgatttacaggtcaagtcccagcttcagagatctgtgctctacattttctgccaggtataaaaacttcatcaggcaatttccctcctccaagggctgtagttctattgataaatagcttcagctacacaggttggtacccaccagccagcaggattttttggttattgctttttgttgttatttttttatatatataccaactggaacatttttaaatctgtactgtatgcacttcctacttctcattgcctgtaatcatccagaataaaaccaggtttcctgatgcacgccagcattagtaactggccagggcaaagaaccctgacagtggcaaacaagcaggatcccccaggcgctggccctagagcctaattgataggtcgtaaggaagtcgctcaagtcaagtacacggttggcttagttctgtctgctctcgtaggagccagtcctttctccactgagaaagcatcttgccagttttacagggagaattcactgatctgacaagccagggaagcttcctgactgacatcttaatttaaatttagcacagaactaagagagtcgtacctctctagcagcataaggaagatgtggggaagtgagctcttcccccctcgctccctgaaaatggaaagcatactgagatggtaaggtaacggatgaaacctgccaccattacaacgtttccctcttcttgacatagcgatatttttagaagagttgcaagcacaaatgaacacatccctgatctggcccaagaactgagacttatttggcactcgtctggaggacgtaaccaatcctccgcagtaagtttccgaggtgtgttccccttataacagaaagtggtttgagactaaaggaaatgggatatccttagtcacagaaaagccaccacccatttcatcgtccagtctagcttcttgaaaagcacagtgtatgataagctatcaaaaagaaaaagagagagagagagaaccctaaagcacctctcagtggtttcctaagcatgctttggtttcaggtttgaagtggttcatcttgcagctaggaagggtgtgtgtgtttgggaacacctcgcacacacacagtggggagaaccaattgtctccttgaaatgtaaaagctgatgacaagggaggtgggctattaggggttttcagagagtcccttataaggttacataaataaaacgagcacttttaaatatcactttaaagattaaaagggaaacaagattagtgaagggggggggctttcaaaatgcttctgtgtttacagacaagggttgtaggtgattagaaaggccatgttcatcatggttgaaatttggaacagctcatgtgaagactgtgtacgctttcatgcaacttgtttaatgtcagactgccattcgagtctggactggaaatggaactctgcactttgagatcagctcattagaatgataacgtgtatatggataaagatgcagtattccttagtctgtgctttgtttattatctgtaccaaagctgcaattgaatagatccggtgaggggaggccttcagttgtaatcacatgccgttgttttgttgtagtgactgcctagaaacgttgctgagatattgttaggaggctgtccagttccttgtattgtgaggattcttagaattgaagcagtgttaataccagcacacacacagtctgcatagaaaggatcgaatgtgagttaatgctggcgaaccacataggcccaggaagccttaatactcatagctcaaatcagagtttattctttaccattgcctgtaagtgtatcacgtaaatgtaggccaccggccagctctctctctctccctttcctgaggttgtatatcccacaaaaatgttttcagattgacttggctcagctaaggaaagagggttggctggccatgtcttccatattccacgtctacttgttcccacactgcctttcacttatctggacagtcacccgcaacacatgcacctgtgtgcacacatctgtatatgcagcagccagcaggcaagcaaatatcaacaaagacctttttttaaagaaataatttgtaataatccatcctatgtagaaatgtaagttattacccaactagagaatcagcttataatactatgtaactttgcctgcgttttatagttaagaaatgtatataaaaaatctaaaatgctatattttcacaatttcattatagagtaatgtgtgtttagcaacccatgcccattggtattttaaatatgctaaatattgaacaattgcaatagaaattcagattttcataagaaaataaaacagaaggatactgcatctttaaaaaacagagtgacgctattttactgtattcgtaacatatgtgatagtggaaaagtattttcaaacatgcaaattttacagaggttgtaaatagtttgatgacgtatgttggggaaaagcttctagttttcatcacaataaaaaaaaaaaaa

SEQ ID NO: 65 - DNA, 3057 bp, Mus musculus
agttccccggccaagagagcgagcgcggctccgggcgcgcggggagcagaggcggtggcgggcggcggcggcacccggagccgccgagtgcccctccccgcccctccagccccccacccagcaacccgcccgtgacccgcgcccatggccgcgcgcacccggcacagtccccaggactccgcaccccgcgccaccgcccagctcgcagttccgcgccaccgcggccattctcacctggcggcgccgcccgcccaccgcccggaccacagcccccgcgccgccgacagccacagtggccgcgacaacggtgggggacactgctgagtccaagagcgtgcagcctggccatcggacctacttatctgccttgctgattgtctatttttataagagtttacaacttttctaagaatttttgtatacaaaggaacttttttaaagacatcgccggtttatattgaatccaaagaagaaggatctcgggcaatctgggggttttggtttgaggttttgtttctaaagtttttaatcttcgttgactttggggctcaggtacccctctctcttcttcggactccggaggaccttctgggcccccacattaatgaggcagccacctggcgagtctgacatggctgtcagcgacgctctgctcccgtccttctccacgttcgcgtccggcccggcgggaagggagaagacactgcgtccagcaggtgccccgactaaccgttggcgtgaggaactctctcacatgaagcgacttcccccacttcccggccgcccctacgacctggcggcgacggtggccacagacctggagagtggcggagctggtgcagcttgcagcagtaacaacccggccctcctagcccggagggagaccgaggagttcaacgacctcctggacctagactttatcctttccaactcgctaacccaccaggaatcggtggccgccaccgtgaccacctcggcgtcagcttcatcctcgtcttccccggcgagcagcggccctgccagcgcgccctccacctgcagcttcagctatccgatccgggccgggggtgacccgggcgtggctgccagcaacacaggtggagggctcctctacagccgagaatctgcgccacctcccacggcccccttcaacctggcggacatcaatgacgtgagcccctcgggcggcttcgtggctgagctcctgcggccggagttggacccagtatacattccgccacagcagcctcagccgccaggtggcgggctgatgggcaagtttgtgctgaaggcgtctctgaccacccctggcagcgagtacagcagcccttcggtcatcagtgttagcaaaggaagcccagacggcagccaccccgtggtagtggcgccctacagcggtggcccgccgcgcatgtgccccaagattaagcaagaggcggtcccgtcctgcacggtcagccggtccctagaggcccatttgagcgctggaccccagctcagcaacggccaccggcccaacacacacgacttccccctggggcggcagctccccaccaggactacccctacactgagtcccgaggaactgctgaacagcagggactgtcaccctggcctgcctcttcccccaggattccatccccatccggggcccaactaccctcctttcctgccagaccagatgcagtcacaagtcccctctctccattatcaagagctcatgccaccgggttcctgcctgccagaggagcccaagccaaagaggggaagaaggtcgtggccccggaaaagaacagccacccacacttgtgactatgcaggctgtggcaaaacctataccaagagttctcatctcaaggcacacctgcgaactcacacaggcgagaaaccttaccactgtgactgggacggctgtgggtggaaattcgcccgctccgatgaactgaccaggcactaccgcaaacacacagggcaccggccctttcagtgccagaagtgtgacagggccttttccaggtcggaccaccttgccttacacatgaagaggcacttttaaatcccacgtagtggatgtgacccacactgccaggagagagagttcagtatttttttttctaacctttcacactgtcttcccacgaggggaggagcccagctggcaagcgctacaatcatggtcaagttcccagcaagtcagcttgtgaatggataatcaggagaaaggaagagttcaagagacaaaacagaaatactaaaaacaaacaaacaaaaaaacaaacaaaaaaaacaagaaaaaaaaatcacagaacagatggggtctgatactggatggatcttctatcattccaataccaaatccaacttgaacatgcccggacttacaaaatgccaaggggtgactggaagtttgtggatatcagggtatacactaaatcagtgagcttggggggagggaagaccaggattcccttgaattgtgtttcgatgatgcaatacacacgtaaagatcaccttgtatgctctttgccttcttaaaaaaaaaaaaagccattattgtgtcggaggaagaggaagcgattcaggtacagaacatgttctaacagcctaaatgatggtgcttggtgagtcgtggttctaaaggtaccaaacgggggagccaaagttctccaactgctgcatacttttgacaaggaaaatctagttttgtcttccgatctacattgatgacctaagccaggtaaataagcctggtttatttctgtaacatttttatgcagacagtctgttatgcactgtggtttcagatgtgcaataatttgtacaatggtttattcccaagtatgcctttaagcagaacaaatgtgtttttctatatagttccttgccttaataaatatgtaatataaatttaagcaaacttctattttgtatatttgtaaactacaaagtaaaaaaaaatgaacattttgtggagtttgtattttgcatactcaaggtgagaaataagttttaaataaacctataatattttatctgaacgacaaaaaaaaaaaaaaa

SEQ ID NO: 66 - DNA, 3263 bp, Mus musculus
ggagaggcgcggcgcggcaggaccaggggctcagcggccgtcggcggacgcgggaagaagtgtctcgtaggcgcggagccccactcagccccggcccccctcgtccgcgtactcggctgatgccccggggcgtcagctcggcggagaggcggtcgaggacgtgcgcctggctcgcagttggctttctctgggactccgacacgtttgcagtcgaataaacttgcgaccgccacgtgtggcatctctccgaggagggcgcgcccatcgtgggatcgcgcggggcgccgggcagggcggcggcgacagcggcggagccggccgcggctgctgcgtgcggaggcgctgccggttacgtaaagatgaggggctgaggtcgcctcggcgctcccgcgagtcgcaagctcccgcgcccccgccccctcggccgcgccgcaccgcgctcgtcgtccgaggccagggcagggctagccgaagctccgcagccaccttcaagtttggccgcaccgcccgggctgccgtcgcccgcaccatgtccgcggccgcctacatggacttcgtggctgcccagtgtctggtttccatctccaaccgcgccgccgtgccggagcacgggggcgctccggaagccgagcggctgcgactacctgagcgcgaggtgaccaaggaacacggtgacccgggggacacctggaaggattattgcacgctggtcactatcgccaagagcttgttggacctcaacaaataccgacccatccagaccccctcggtgtgcagcgacagtctggagagtcccgatgaggatataggatccgacagcgacgtgaccaccgaatctgggtcgagtccttcccacagcccggaggagagacaggattctggcagcgcgcccagcccactctccctcctccactctggagtggcttcgaaggggaaacacgcctccgaaaagaggcacaagtgcccctacagtggctgtgggaaagtctatggaaaatcctcccatcttaaagcccattacagagtgcatacaggtgaacggccctttccctgcacgtggccagactgccttaaaaagttctcgcgctcggatgagctgacccgccactaccggacccacactggggaaaagcagttccgttgcccactgtgtgagaagagattcatgaggagtgaccatctcaccaagcatgcccggcgtcacaccgagttccatcccagcatgatcaagagatcaaaaaaggctcttgccagccccttgtgaggtgctccccatggcagccaggcagagatgggtccccggaaggacagagctcccaggaaacagactgacacatggaaatctgccacagcagaggcgcgctggccacaggaggtcactgcttctttggccaatattctgatatctccctgcactgtttccaaaaagcacatggtagccctaaggtcaaagtcaacatttggtccccttgcagaggcaactctgaaccgtctctgactgttggagggcaggcaaataaatgcttttggtattttctcctttgtttttgggggaggggggagggggcaaggccaagacgaggggtaggattctgaagattcagactggtggtgtacatacgtctgactgggtgagttgacccctggcctcccacagtgactctggatccttgatgcttgctgactctcagaattgttttttcttctcatttaatgtaacgaccggtgtgcttcagtttgtttagcagaaccactctcttgaatcacattaacttttgagatttaaaaaaaacaaaacaaaaaaaaaaaaaacaacaaaaaaaaaccaaccctccatagcacagctgtcttttatgcaagcaagagcacacctactccagcatgatttgtcatctaaagacttgaaaacaaaacaacaacaacaaaaagttacttatagtcaatggataagcagagtccgaatttacactaatcaagacagaccttcgaggggtcacgataagtccggaactttcaaaccttgcttcgtatgaattgtactatctgaacataaactgcacttttattttctaataccgagggtgaatacggtaaatacatgctttgagggtagaagccgacggtctgtttggcaccacgttataatctgcttattttaacagtatacacatttccctgagaacctatggcaaagatgggagggcagaacatatgaaacacatgctgacgagggggaggaggaggggagtgcttttaaagataacaaaaaatgagcaaacaattttaacttcatgaatttgtttgaattcccccatgctttctgataacgccgtctcgttccacgctcgggactaggacagagcgagagcggtcggctggctctctgtagcgttggttgataaatgggtatatagccatcaccaggcctggaagtgccccccctttggtgggggggtgcttcttggtctccttatgttctatgaaattcccactatgaggtgcaagagttatatcaaaggaagctgaaatacgaaaaaaaaaaagtttgcccctgtaagtagtaagtgtctagggaaggaagacgccacaatgaatgacttcagaggccactctgcagaacagtcccttctggtgaacataggtcaaagaaacgcacgtgaatggcatcacatcactgtctgcatcagtatccctaccactacttagcttgtatctaggtgtctacacaatggcctgaaccactatatccatcctttgccatttactgaaaaacttagttttggcttttacatggccagaaaaacaaactcagttttcatagttaaaaaacaaaaaaagaaaacaacaaaaaaaaagaaaacaaaaacctcatttgaggggagtcagcagtgatcatggggaaatgtttacattttttccttcagaaataccactttctgatgattttatctgatatttaaaacagggagctatggtgcactctagtttgcacatggtgtgtaaactcggggttcccacctgcagtgagctccacatttcacccagcgcatgctcacccccatctcagcgtggttgctcctgcagtgggcacagggcacagctgactccgacttccaatacgacagccatcactagcgcagtgttttctgtttaaccaatgtagttgtatcagtagttctctcagaactgcttttaacattagggactgggagcagtccacgagctaaaaaggaaagtgttttctcatgagaaaacatgtcagg

SEQ ID NO: 67 - DNA, 1605 bp, Mus musculus
acacagcttgcgcccctctctggagagcagtcgcagctactagcttgccgccagcagagccgcactgtctcgggcatcgcagagcaccacgactgtggacttgggacctcgaacccgagacgagcgcgcgctagccagccattagcaacgcttgcatttctgcagggagcgcggtgactgtccttgagcgtggaggggcgagctcgccagcggagcgaccgagcaagaggaggcgcaggagcggcggcgcccgagcacccgagggggtccgagtccaggcagctagccagccacgcgccacaaagggagcgcccccgccgcccggcacgccgcccccctccccaatgtcctcggccatcgaaaggaagagcctggacccgtctgaggaacccgtggatgaggtgctgcagatacccccatccctgctgacatgtggtggctgccagcagaacataggggaccgctacttcctgaaagccatcgaccagtactggcatgaggattgcctcagctgtgacctctgtgggtgtcggctgggagaggtggggaggcgcctctactacaagctgggacggaaattgtgcaggagagactatctcaggctttttggtcaggatggtctctgtgcatcctgtgacaagcggatccgtgcctatgagatgacgatgcgggtgaaagacaaagtgtatcacctggagtgtttcaaatgcgccgcctgtcagaagcatttctgtgtaggtgacagataccttctcatcaactccgacatagtgtgtgaacaagacatctacgagtggaccaagatcaatgggatcatctaggccagagtcccaggcacctgtggggaggtgttcagtgaagacgccgtctcggtgtggtcttcactcttaggctctttgggaacctgagggtgggtgaggcatttcttaggagattatggactcttcctgggcactaacgacatagcaatcacatgacataatgcaaaggctgagacttcagtgacaaaaaggaccagcccttagagagaatttatggtcacaggctagccatagtaactgacaagattagtggaggaaatatttggggaccagcatgcactgtgccatcctgataggcttccccaggtaactatcgattagatgtgaccttggtgttcatggactagagttgtgcaattaattttttcctggctttggtaactaccctgttctagtcacagtccttcacactaggaaaggaaagagagaagaaatagtcactctttttttctttctttcttttttccttggtcaccttcccgtggccttaagctctggagccaagggaaactgcatggaggtacatttcagttgggaatgaaatccacatctttcaaccagacaaatatttatagcgatgttgatgaatcactgtttttagacaccttcattttgaggtgaggcgccccacagattgtttctatacaaatgtaaatcttaaaaaaaaaaggtcaaagttgttcaactattttattatcttagattatatcaaagtatttgttgtgtgtcattaaaaaaaaaactcgattctgaagacttaataaaaatgacaagctgaaatgca

SEQ ID NO: 68 - DNA, 3441 bp, Mus musculus
agcattctggtcggaatccacctctccgcctgtgcaacacacactttacacacgcacggcgactgcaagcgggcagcatcgatcgtggctcctttaagacaaactcagacagacatttttttaaccctcctctctaatctcccttcagtgcagcagttgcaaagagggagagagaaagagaaagagagcgagagaagagagaaactgattaggaattaggactgattcaaggaaagcgggcgctagggctttgtgcatttgaatattaacatttgaggtgttctgaccagaagaagacagaacggacgatcattcattcaccacgttgacaacctcgcctgtgattgacagctggagtggcagaaagccatgagatttggtagttgggtctgaggggcgctcttttttttttttctttttcttttcttttctttctttttttttaaactgatttttttgggggggagagaagatctgctttttttttcccctcccactgctgtcttggtggaaccagagcgcttttatgctcagcgacgcgggcgccttgcttcaggtccggtagaccgaagatctgggaccagtaattcacactgctggagacgcaaagggatttttttttgttattgttgtgctttatttttttttccgggggagtttgcatatttgtttcttttcacactggccttaaagaggatatattagaagttgaagtaggaagggagccagagaggccgatggcgcaaaggtacgacgacctaccccattatgggggtatggatggagtaggcatcccctccacgatgtatggggacccgcatgcagccaggtccatgcaaccggtccaccacctgaaccacgggcctcctctgcactcgcatcagtacccgcacacagctcacaccaacgccatggcccccagcatgggttcctcggtcaatgacgctttaaagagagataaagatgccatttatggacaccccctcttccctctcttagcactgatttttgagaaatgtgaattagctacttgtaccccccgcgagccgggggtggcgggcggggacgtctgctcgtcagagtcattcaatgaagatatagcggtgttcgccaaacagattcgcgcagaaaaacctctattctcttctaatccagaactggataacttgatgattcaagccatacaagtgttaaggtttcatctgttggaattagagaaggtacacgaattatgtgacaatttctgccaccggtatattagctgtttgaaagggaaaatgcctatcgatttggtgatagatgatagagaaggaggatcaaaatcagacagtgaagatgtaacaagatcagcaaatctaactgaccagccctcttggaatagagaccatgatgacacggcatccactcgttcaggaggaaccccgggcccttccagcggtggccatacttcacacagtggggataacagcagtgagcaaggtgatggcttggacaacagtgtagcttcccccagcacaggtgacgatgatgaccctgataaggacaaaaagcgtcacaaaaagcgtggcatctttcccaaagtagccaccaatatcatgagggcgtggctgttccagcatctaacacacccttacccttctgaagaacagaaaaagcagttggcacaagatacaggacttaccatccttcaagtgaacaattggtttattaatgcccggagaagaatagtgcagcccatgatagaccagtccaaccgagcagtcagccaagggacaccttataaccctgatggacagccaatgggaggttttgtaatggacggtcagcagcacatgggcatcagagcgccaggacctatgagtggaatgggcatgaatatgggcatggaggggcagtggcactacatgtaaccttcatctagttaaccaatcgaaagccagggggaaggctgcaaagtatgccaggggagtatgtagcccggggtggcccaatgggtgtgagtatgggacagccgagttatacccaagcccagatgcccccccatcctgctcagctgcgtcatgggccccccatgcatacgtacattcctggacaccctcaccaccccgcagtgatgatgcatggaggacagccccaccctggaatgccaatgtcagcctcaagcccctcggttcttaacacaggagacccgacaatgagtgcacaagtcatggacattcacgctcagtagcttaagggaatatgcgttgtctacaatggtgactgatctcgaatcaggtcttttcctgcaaggactatggagttccattcttgacatctactttggaccaaggagcatccctagttcttcatagggactcttaaaatgcaggagaaccatccgaagtcaactcgggggacatgcaaaaataactatataagacattaaaagaacaaagagtgaaatattgtaaatgctattatactgttatccatattacgttgtttcttatagattttttaaaaaaaatgtgaaatttttccacactatgtgtgttgtttccatagctgttcacttcctccagaagcctccttacattaaaaagccttacagttatcctgcaagggacaggaaggtctgatttgcaggatttttagagcattaaaataactatcaggcagaagaatctttcttcttgcctaggatttcagccatgtgcgtgcgctctctctctctctctctctctctctctctctctctctctctctctctctctctctcttctcctccctctccctctttctagcctggggcttgaatttgcatgtctaattcatttactcaccatatttgaattggcctgaacagatgtaaatcgggaaggatgggaaaaactgcagtcaccaacaatgattaatcagctgttgcaggcagtgtcttaaggagactggtaggaggaggcatggaaacccaaaggccgtgtgtgtttagaagcctaactgtcacatcaagcatcatcgtccccatgcaacaacaaccatcaccttatacatcacttcctgttttatgcagctcaaaaaacatagactgaagatttattttttaatatgttgactttgtttctcagcaaagcattggtcatgtgtgtatttttccatagtcccaccttggagcatttatgtagacattgtaaataaattttgtgcaaaaaggactggaaagatgaactgtattattgcatttattttgtttttttttttgtgaaagtagcagattagtgcaaggtggcatgcatataaggttaactgagtggggtacgctaattatactttttgttgtggataaaaaatgcttgtcaatagcctttttctatcaagaaaacaaggagctaattattaataacagtcatggcacactgtgtcctatcttagcatttaaaagtatgcaaactccaggtccag

SEQ ID NO: 69 - DNA, 6505 bp, Mus musculus
ggagcttcgcagccgccgggcggggggaggaggcgggggaggagggacggagatctggggctcggagccggccgccgctgcgctccgcttcgctgtggggctcggtgtgtcgggggtgggggggcgggggggctccgctatggaggcaaatgggagcccaggcacctcgggcagcgccaacgactcccagcacgaccccggtaaaatgtttatcggtggactgagctggcagacctcaccagatagccttagagactattttagcaaatttggagaaattagagaatgtatggtcatgagagatcccacaacgaaacgctccagaggcttcggtttcgtcaccttcgcagacccagcaagtgtagataaagtattaggtcagccccaccatgagttagattccaagacgattgacccaaaagttgcatttcctcgtcgagcgcaacctaagatggtcacaagaacaaagaaaatcttcgtaggaggattgtctgcaaacacagtagtggaagatgtaaagcagtatttcgagcagtttggcaaggtagaggatgcgatgctgatgttcgacaaaaccaccaacaggcacagagggtttggctttgtcacctttgagaatgaagacgttgtggagaaagtctgtgagattcatttccatgaaatcaataataaaatggtagaatgtaagaaagctcagccgaaagaagtcatgttcccacctgggacaagaggccgggcccgggggctgccatacaccatggatgcgttcatgcttggcatggggatgctgggctaccccaactttgtggcaacctatggcagaggctaccccggatttgctcctagctatggctaccagttcccaggcttcccggcggcagcttatggaccagtggcagcggcagctgtggcagcggctcgaggatcagtcctgaatagctacagtgctcaaccgaattttggcgcgcccgcttccccggcaggctccaacccggcgcggcccggaggcttcccgggggccaacagcccaggacctgtcgccgatctctacggccctgccagccaggactccggagtggggaattacataagcgcggccagcccacagccgggctccggcttcggccacggcatagctggacctttgattgcaacggcctttacaaatggataccactgagcaggcgcttccattgccgtctcactatgagagcatacctggatgtccaggcaagactgggcgaagtttctgagtggccctttgtttaggtgacgtcctcagacctggacccccaccagcctcactcctcatcccaaccagaggtggcacacttggattgagggttgacacatctcatctcacccatcggctacctgctgtaatataagacaacagcttttaaacgtgtatataatccatgattttggtttggttctgtttgttttccttggtggtccccctctccctcttcccttctccttttaaatctccctcaatcacatttggtagtgatttttgacttagtctggtagtcacccagcttaatatctagttaaagctaaccatagtatacttgttatatattaaggagttttttttctttttttttgttttcttttttcctttaaagagaatttttgttttgttttgattctgttctcgcttttaaaggatgctgagatggtgatgttactctccatttttggtaccagttctgagactgtaagacttttcatctgggactttagcacacactgaatcgaagttgtgatacgcggagcgggaggtgggcatagactctattttgtgttgtagaagtgacatacagttggctgcttaacagactctctagccgttcatttttgtgacgtctctttgttaacctaagtatatctattttcggcaataaggtaaggacggccgtgttttgagggtcttcctttcctatgagtgctttttcttttcttctgttcaaagaggtcatattacaaggttttttgaaattgtgaattctaaaaaacaaaaaacaaacaaaaaaatgttgtaaatataattccattaactacatagaaactattaaggaagagagaatcaaaaaatatttttgtgagggagttgatcctgggcagttaggtaccctggaaggaggggagggaacctcgatgaagccagtacaacaacagaagagcccccccagctgccagggcaccccagacccccactcttcactgttggtgtgatagacctaagaaccacattagcaccctgccagcctaccagcctctctgtgcacagcttctaatgttatgatctagctagatttttatttaaaatatgaaaaactgcttttcccaagacgttttttaaaaggaaaaaaagctacaattttaatatttgacatatttaaagtttcaaagcacacttgttttggcttaggggaggggcagggtggggtcattctttttcagtcttaatttttaaatatctgatcattttatattgtccgatcatttcagcacctccaaagggtcctaggatattctgcctctctcttcaccctgtccccagacgggggaacccgtgggtcaggcgtggataagcctgcgagaatacaccctttctccattcactttctatttacaaattaggaaagcaacctttcagtttatatatatattttttttcctttttgatacctcagtgctgcaagtatcatcagagaggctatggaagagttttgtgttttgttttttgttttgtttttttttttaatttattgtagatgtaaacagaattttaaaaataaaatataaacatcactgcactgtgactggtggggaaaactgacagcttcctgtttgtacacatttgacattggctgttataatatatggtcctcagttggggaagacacttagaatgaaggatgttttttaatttaacttttttttttttttaaagaaaaatatcagtaataggtcggcaacagcagccgtaggaagtacaacttagggtagcattaaagcatactgtagtgtggatatattttttttcttttttaaaatgtgatattgacattttattaatattttttaaattgttacgtttataaatttggtacttaaggcacagccagtgtgaggcactgaacgcgacatttattacaatgagctgctgcactcctacttttataaattttactaacaagtagactaatgtagacattcacagacgggatagcgcagaagcatcttcacacgacaagtctccaaaaaaaaaaagctaattcagagagggcgggaggaagcctgttcccgttctaattaagtgcaaccttgttttatcttattgttttattttttaagaaaggaaatcatgttctttttttatatctctatatataagatacagagttgtttggtaaggtttggggtgttttttgttttgttttgttttaagtttccaaacaaccaaactaagaaaaatgctggatgcttttagaatacaaaacttactcaagtcataaaataacaaaatagaacttttactttaagaaacaacaacaaagagagagagagactcgattcccagcaagtcagtgtctcacaagggcactggctaattactcttctgtccgcgttgctgatgtcctgtccccagcccctgtccatctcccccacactcccatcacctcatagaacgctctttgttgatcattgtctgttaataatgtataaaatggctatcttgtaagtgtgctgtcttggtactagtgtagtgactttttttctcctcttctagtacatattgataggtataatgtaattaaggttttaaaaaaaatagacatagttattcagattaggaccagtaaggatagaactttctcttatttatgaaaagaaaaaaaatgctaataatttgggggcagttttttcctttttttttttttccaagttcaaatttacttttatttttgctgatttgatgtggtttcaactaacccaaggtctcacaatgttcaaatggcggctgactctacagcattctgtaggtccctgcccccctagctgaaggggtgtgccatactaccttaaatgcaaacactagatatgcaaaactggatttttttaatttattttttaaaagagggaggtgtggtatattaaaatgattttactaagagaaaaaaaatatttttttaaggatgctcagaagaaattgataatctgtgtgaatatgttttagatgtttatatacattttgaagagacccagtagcccatagcacaaatcttgtgaacatctgatatgttttcaagtggctacctaggataaggttcatcattagtacccccacccccaccccaccgccatctagaagtccatcttaaacaattttttgtaaattctttcagcactggtgtccatctttgtctttgtttcagttaagctcaatagcgaatgtgggaccccctcctctgaccttccctgggggagaaaccctcttggctaatggctcttccctggcattatcaataaccacccggggactctctggctttcagatccatctgcctgagaccccaaggtcctctcctccctagaggggaggtggagcagcagttgactcctggttccctccctatttcagcctggatgtggggctggtggagatgcctccaccccaggagcctctgcatatgtggttcgtggccttcttctcacccattgggaaaaccaaacagcctcacactctgtccccatcgctcattggcttaactcaagtgagaccccaactgggcctttgcgttttgtttttgtttttttttaaatccttcatgaccattctcttaatttgaactcgtagcttgggctttaaggtagcatggctcgattgctgtcgacttaatgttccactgcacagcaattcacggccagtgaatgttacacacatcttgctagactagtataaaaatcattgggtaattgttggttctaatgacctgaaaggtgttcagtttgtgggttttttttggggggggctgcttggggggttgtttttccttctttgtttttttttaatttgagaatttagggggataatttttgggggggggttccaaataaaaaaatagaaagctattttgatctttagtgcaaactagggacgtagcctccatcaccacaaccacaccgcttcgctctgccatccacccaccgcagcagcatcttcaagaataaagcaatatagtttactacatttttttttaaaattgaaggtcagccatgctttctgtattatattgcatatgaaattgtttacaaaagaaacactaactcatccttctctttatcagttgaaagtgacacacgggacagagggaaattggagggcaggtgagggaggggagagggtttttttgtattttggtttgtttggttttggtttcttttcttgaatgtgctttagtagccagatcctccaaggaaggcaaaagccagccagccagagagagagtagcgaagcgggcaggctgcaggtaagcacactgaacaagagacacactggcaagctcccccacctcagcgagagcagaaagcgaacagctcagcccagtcctgggcacagacactacacaaggcaatgctggaattgaaacaatactttaataccgtttaagtttgtttcctttttttttttcctcttttttttttctttcaccaagaaaagaaaaaagtaaactaaaatacaaaatacaaaaacaaacaaacaaacaacaacccatataaataaaacccacccctctctgggaaacaaccataattcaaacatggctatttagtaatcagaaggtattgtctcagacaggatttcatttccgggaggcagggcgtgaggggggaggggagtgggactgagagacagttccagagcctccagggaaggcttctactgctaactgctgtattctgtatatactgtgccaccctgtgtggagtctgtgagtgtgctcttgagtagcgtgggctagccaatctgccattcatggtgttataaactcggaattccatatgtaataggatgcaagtctaagcgtttcatgtggacataaatgtatctaaataaaacttcccctagcactgtggctgacctcacccttacttttatactttagtatgaaactgatgagaactttggtagtgagtattttttttatatatatacatatatatgtatctatatatatatatatatctcaagcatctttcaggtctttgtgtgtggctttcttaaagccctgttgtaaaaaaaaaaaaaattactaagtggatggcagtctctcacatcacagatgtggaaagtataattttatatttgtattttcaaataaataagtttgtgaaaggtttccatcctctactgtggtccagaaatcaatgtgtttgtctgacaaaaaaaataataaaataaactgttttgaacagaaaaaaaaaaaaaaaa

SEQ ID NO: 70 - DNA, 2596 bp, Mus musculus
agtgacagtcatctgtctggacgcgctgggtggatgcggggggctcctgggaactgggttggagccgaacgagcgctagccaggcgtaagcgcgcacacactgcagccgccggaggacaaccccctcccgccgccgctccctcagcccacccggagaccccagccccgagtcgcctccggatccccggcagtctgcgggagagttggaggttggcgcgactctgctgctctccacgggaaggaagcactcccccatattaaaaagagcggagatattaaaagagaggcgaacccatgcccagctgcaccgcgtccaccatgccggggatgatctgcaagaacccagacctcgagtttgactcactgcagccctgcttctacccggacgaagatgacttctacttcggcggtcccgactcgaccccaccgggggaggacatctggaagaagtttgagctgctgcccacgcccccgttgtcgcccagccgcgccttcccagagcacagcccggagccttcgaattgggctacggagatgctgctgccggaggccgacctgtggggcaacccggccgaggaggatgcgttcggtctcgggggcctgggtggcctcactcctaatccggtcatccttcaggactgcatgtggagcggcttctctgcccgcgagaagctagagcgcgcagtgaacgaaaaactacagcacggccacgggcccccgggcgtcagctcagcctgctcggctcccggagtgggtgccagcagccccgggggccgtgcccttggtgggtcgtcgagtgctagccacaccggggccaccctgcctaccgacctctcccacccggctgccgaatgtgtggaccccgccgtggtcttccccttcccggtgaacaagcgagagtcggcgtcggtgcccgctgcccccactagcgccccggcgaccagcgctgcggtcactagtgtgtctgttccagctactgccccggtggctgctcctgctcgtgcaggcggccgtcctgccagcagtggggaggccaaggccctcagcacctccggagaggataccttgagcgactcagatgatgaggatgacgaggaggaagatgaagaggaggaaatcgatgtggtcaccgtagagaagagacgttcctcctctaacaacaaggcggtaaccactttcacgatcactgtgcgtcccaagacctccgctctgggcctggggcgagcacagcctggcgagctgatcctcaagcgctgtgttcccatccatcagcagcacaactatgctgcaccctcaccctacgtggagagcgaggacgcgcccccgcagaaaaagatcaagagcgaggcttctccacgccccctcaaaagtgttgttccagcaaaagcgaagagcctgagcccccgaaactcagactcggaggacagcgagcgccgccgcaaccacaacatcctggagcgtcaacgccggaacgacctgcgctccagcttcctgacgctcagggaccatgtgcctgagctggtgaagaacgagaaggccgccaaggtggtcatcttgaaaaaggccaccgagtacgtgcacgccctacaggccaacgagcaccagctcctgctggaaaaggagaaactgcaggcgaggcagcagcagttgctaaagaagatcgaacacgctcggacttgctaaacgtttcccacacggacagtcactgccactttgcacattttgatttttttttttttttaaacaaacattgtgttgacattaagaatgttggtttactttcaaattggtcccctgtcgagtctggatctgggtagggggcaggacacggggttctgccatgaccttggaaaaaaaactgacttatgggatgctgggtggcttgttttcctcctccatatcacctggtgacagccgtggaagttcgggacactaaggagcttcaggaggctgtgaagtcaccttgttccggtccaagattccaaacagagtcattccttctttttacaatggtgcttaagttccagcaaatgccacagaagggggggttgccatttgatgcccctgggaacacttgtgtaaataccattgatacacccgccttttgtatacgtcctgggtaatgagaggtggctcttgcggccagtattagactggaagttcacacctaagtactgtaagaatacctcaatgtttgaggggcatgttttgtatacaaatatattgttaatctgttatgtactgtactaattcctacacggcctgtatactttagtatgacgctgatacataactaaatttgatacttatattttcgtatgaaaatgagttgtggaagttttgagtagatattactttatcactttttgaactaagaaacttttgtaaagaaattttactatatatatatattcctttttttcctagcctgtttcttccttgtttactgtatttgttcatgtttggtgcatagaactgggtaaaatggcaaagttctgtgtttaatttcttcaaaatgtatatatttagtgctgcaccttagagcactttgaaatacctcatgtttatgaaaataaatagcaattaaatgatgcaa

SEQ ID NO: 71 - DNA, 2861 bp, Mus musculus
agacggttctgagtccggtactgcgtcactggactgcagcaggtctcggcgcaggtccattgcggcctgacaagcctcgtagctaaggctccaggctgcttcccctgctgcggtttcctgcgcgccacctctgtgaccccgcccccatcgcgccactgccacagctgctgcagtccgtgttgccactggatcccggccgccgccgctgcagtctccgccgcctgaccttctccggccttcgcccctgctgccaccgccccccaccgccgccgccactggagcatccgtgagtccttttctgtccgtctctgggtgcacgatctctgctgttgtcaccaccttaaataggtgtacggggctaacagtatggcagaagcggatcctaaaatggtcacagaacctggtgcccatggggttgctgaagaggcgatggctagcacagcttgtgattctggggatgaatctgacagcaatagctctagcagtaccaatagttgcagcagcagcggcagcagcagcagcggcagcagcagtagcagcagcagcagtagcagcagcagcagcagcagcagcagtagcagtagcggtagcagtggcagcagcagcaatggcagtcatttgaaccgaaagaagagggtacctgagccttccagaagggcccagcgacgtccctccgggaaacttttcttggataagctgccccaagccgtaagaaatcgggtgcaggcactcagaaatattcagaatgagtgtgacaaggtagacaccttgttcttaagggcaattcatgatcttgaaagaaagtatgctgaactcaataagcctctatatgataagcgttttcagatcataaatgcagaatatgagcctacagaggaagaatgtgaatggaattcagaagaagagttcagtggtgatgaagaaatgcaggatgacacacctaatgaaatgccacccttagagggtgaggaggaagaagaaagctgtaatgaaaaagctgaagtgaaggaagaaggaacacatgttccagaagaagttcctgaggcaaaagttgaagaagaggaggctcccaaagaaactcctgaggtgaaaactgaagaaaaagacattccaaaagaaggtgctgaagaaaaagctgaagaacaggaatcctctaaagaaattcctgaggtaaaaggtgaagaaaaagcagactctacggattgtatagatatagctcctgaagaaaaagaagacgtcaaagaagttacccaggcaaatacagaaaataaggatcaacctacagaagaatttacaccaagggctccagcaagagaggctcaaaaaagggtccctgagacaaggcctgaagaaggagtcaatattaaaagggctcgaaagggaaaacctaagaaagaagatcctaaaggtattcctgactactggctgactgttttaaagaatgttgataagcttgggcctatgattcaaaagtgtgatgaacccattttgaagttcttatctgatgtgagcctgaagttctcaaaccctggccagcctattggttacacttttgaatttcatttcctacctaacccatacttcagaaatgagctcctgatgaagacatacataataaggtcaaaaccagatcactacgacccgttcttcgcgtggggatgggaaattgaagagtgtaaaggctgcaaaatagactggagacgaggaaaagatgttacggtgacaaccacccggagtcgccctggtattacaggggaaattgaagtccagccaagagtggttcctaatgcatccttcttcaatttcttcagtcctcctgagattcctttgattgggaagctggaaccaagagaagatgctatccttgatgaggactttgagattggtcaaattttgcatgataatgtcatcttgaagtcaatctattacttcacaggagaaatcaatgatccctactaccatgacttcagggattatggaaataggaagtactacaagtagaaagggcaagctgaaaaatctttcaagaatccttaaaagttaagcaagcagaagtgaaataagagtcatctaaacttctaaaatagaaaacttgccccataacctgaacagtattactccttatagtctggtgttttcattttttcctggtaatgtcctaaaaagtgtcttaagcttctgtttattctatcttgcctgttgtattgtaatattcttcaaaatatgtaaactgctgttaatttccaaagcatgttttgtttggtgctacagtgttggttttgtgaagtcagtttttgtcatgttgctattagattgtacctgtgaaaattgtcagatttattagagaaatccttgcctgaataagcaacattttaaatgccaatatacgtgtgttttctctttctttttttacagcccagaagactaaagacttaaatctgcttgcactagatgtgccttcattactttgccatagaaatgtagtaataataacaactgaaacactctattgtgaaatgtagttgcttgaaaaaaaaagatgagaatatatttaaaaatgtcaggatcaagtagatttttgtgggtttgtttatgtgatttattggtctgttagcaaaagtattgtttaccactaaggtgttaaatatggtatagtattttaaaatttttctttgttgtatattgtgtaattgaaaactaaagaaaccgtattttctttgtaatgtgttctacattgccttttaaaaattatttcatcatttgatacattatcatgttcttttattgtacctggagtaaatatcaaataaactgccatgctataaaaaaaaaaaaaaaaaaaaaaaaaaaaaaaaaaaaaa

SEQ ID NO: 72 - DNA, 1652 bp, Mus musculus
cccgcccgcccgctgctgcggaaggcgcagtgctcagtaaagcgcacttcctctgctggtctccaccgagggagtgcccgctccaagagctccaagccgcatcggtcctgctctgatccgaaggcgcagacatgtcggaacaaagtaaggacctgagcgaccctaactttgcagccgaggtccccgactgtgagatgcaggacagcgatgccgttccggtggggatccctcctcccgcttctctggccgctaacctcgcagggccaccgtgcgctcccgaaggccctatggcagcccaacaggcctcgccaccgcccgaagaacggatagaagatgttgaccctaaaatcctgcagcaggccgcagaggagggccgcgcccaccagccccagagtccagcccggccgatcccagcaccgccagcccctgcccagctggtgcagaaggcgcacgagctcatgtggtacgtgttggtgaaggaccagaagaggatggtcctctggtttccagacatggtgaaagaggtcatgggcagctacaagaaatggtgcagaagcatcctcaggcgcaccagcgtcatcctcgccagagtgttcgggctgcacctgaggctgaccaatctccacaccatggagtttgccctggtcaaagccctcagcccagaggagctagacagggtggcgctcaacaaccgtatgcccatgacaggcctcctgctcatgatcctgagcctcatctatgtgaagggccgcggggccagagagggtgcggtctggaatgtgctgcgcatcctggggctgaggccctggaagaagcactccaccttcggagacgtgaggaagataatcaccgaggagttcgtccagcagaattacctgaagtaccagcgtgtgccccacatcgagcctcccgagtacgagttcttctgggggtccagagctaaccgtgaaatcaccaagatgcagatcatggagttcctggccagagtcttcaagaaagatccccaggcgtggccttcccgatacagggaggctctggagcaggccagagctctgcgggaggctaatcttgctgcccaggccccccgcagcagtgtctctgaggactaaaaaggtccaggggcacactgatagtttctgacccatactagggctgtgtaagggtggggttgagtcattagagtatcccaaatccacagtgcagtatttcatgtataatttttaagttttccatacagtgcttttgtaccttgtaatgctattcatttgtgtactcgtgtagtgtttaagattgatgcatgtgtgataagtatttggtactttcacttttgtgctttcgtgcatttttgtacaagagatgtgctgtgctaaacttgtgaaatacattgaggtgttctgtatcttgttctttgtatgggactgatgatctgtatcgacaaagaaggccctggagagttagcaggacttaacagcaacgcagacctgagcaagagaaaggtcaaggcctttctccatatgacttcaactggcacaggaagcatccatgtggaatggactgatttgaactggactgttctcagtgtaggcacttagcaccctttacaaaacatgtatgcaaccccaccataaataaacgttaaaatgagcattaaa

SEQ ID NO: 73 - DNA, 3565 bp, Mus musculus
gattctctctctctctctctctctctctctctctctctctctctctctctctctctctctctgtttttctctcctttcctctggctgtctccctcccccttttaactgacttggcacacataaatggatggggattgagcatggaaggggagggggagtttgagagagaaaaggagccacaacccccccacccacccacccgagcagtgctggtgcacacacaccctctcccgcgcgcacacattcacatgcgcaccatcgcacaccccatcccatccccgtcttcctgagatcctcgctctctccctccccctcttccttccctccctccctggctccctctctccctttcgctgcgtctgccagcaacggtctgcagccggtcagaactcgtcctcttccccgagaatctgcgagctccccctcttcctctggtcgggtggagggagcagctcgaagtttacacccttgtgccgctgccaaagccgaaagccttttcttcagctgctgcttttttcctcctggtttttgtttttgtttttccgggggttgggggggtggggtagggtacgctgttgtgggtgtgcgtggggcggtggtaggaggttggttttgatttttaattttgcatattttttttgctcttttattttaaactgcaagaggatgcacagaggaagaaattgaaacaatttgttagcttttgtttacctggtgtgtgtagaagctacctcgcgctctctctgctcgccatcctgacctttctttctttctactccttaaaaaaattaatttccccctctgtgcaatggagcgtggggggcggagggggaagggtttgagaatccacccaagcccggcccctattccccagaacaccaataataaccccctttaaaacatttaccttcctcccctgctcctcctcctccccaagtcccaccgccctaactcataactctgttgagcccagaatctcagaatcaggcgttggctttgccgggtgcttcagatcaatggatgagttccacccgtttatcgaggcgctgctgcctcacgtccgagccttctcctacacctggttcaacctgcaggcgcggaagcgcaagtacttcaagaagcacgagaagcggatgtcaaaggacgaggagcgcgcagtgaaggacgagctgctgggcgagaagcctgagatcaagcagaagtgggcatcccggctgttggccaagctgcgcaaagacatccggcccgagttccgcgaggactttgtgctaaccatcacgggcaagaagcccccctgctgcgtgctttccaaccccgaccagaagggcaagatccggcggattgactgcctgcgccaggctgacaaggtgtggcggctggacctggtcatggtgattttgtttaaagggatccctttggaaagtactgatggggagcggctctacaagtcgccccagtgctcgaaccccggcctgtgtgtccagccacatcacattggagtcacaatcaaagaactggacctttatctggcttactttgtccacactccggaatccggacaatcagatagttcaaaccagcaaggagatgcggacatcaaaccactgcccaacgggcacttaagtttccaggactgctttgtgacgtctggggtctggaatgtgacagagctggtgagagtatcacagactccagttgcgactgcatcagggcccaacttctcactggcggacctggagagccccagctactacaacataaatcaagtgaccctgggaaggcggtccatcacctcccctccttccaccagcagcaccaagcgccccaagtccatcgacgacagtgagatggagagtccagtagatgatgtgttctatcctgggacaggccgctctccggccgctggcagcagccagtctagcggatggcccaatgacgtggatgcaggccctgcttctctaaagaagtcaggaaagctggacttctgcagcgccctctcctctcagggcagttccccacgcatggctttcacccaccacccgctgcctgtgcttgctggagtcagaccagggagcccccgggccacggcatcagcgctgcacttcccttccacgtccatcatccagcagtcgagcccgtatttcacacacccaaccatccgctaccaccaccaccatgggcaggactcgctgaaggagtttgtgcagtttgtgtgctctgacggctcgggtcaggccaccggacagcccaacggtagcggccagggcaaagtcccggggtcatttttgctaccgccgccgcctccagtggccagacctgtgccccttcctatgcctgattccaaaaccaccagcacggccccagacggcgccgccttgactcctccatcaccttcattcacaacgacaggcgcctcctctgccaaccggtttgtcagcatcggatcccgggacggcaactttctgaacatcccacagcagtctcagtcctggttcctctgattaagatcaacaaagaaacaactaaatttaaaaaaggaaaaaaaaaaaagaggttcctcaaaatgggggagaagaaattttgagacatggaaatatcccccagcccagccccaccgaaaagcaaaacttagacgtcgtcagccactcagcccttccctcctccagccctggtacccccgcgttccaaagccgcccagttctgggagtcctcggaaaggggtctcagcggagctgtacaccagcagccaagcagaaagaagcatgctaagcggactctgctaagcagaggacaaacagaaagaaaggatgcaacaagactgccttcctcctactcagcctggccttcccaggaaggagcagccagctcactggccggccaccctggcagttcctggggaccccaccctccgctggacttaagcttcccaaggagcccagaggaacagctcgtaacaatagtagacattcgagctcccctccggacttcgtctctttccctcaccccaccccccactccccaacctctcttgcccttcctgggagcctctgctcaggcttctggccccagctgctctggaccaggctagggcgttcccgggtaggtaagggatgcttcttagagctaactgaattcagcctgagtccagaatagagggtgttctgccaccccattttgaaccgcccgggcagttctcgggctggacaccctctgtacagcacacaggaaagtcagaatgctctggactggacgcccagcccagcataggcaccttcttccacgcccgcaggacgcaagggctgctgccctctgccacgcccctagtgaagggagtcggccccaggtcgccccgcaccccagccctgcatgcaggtgcccttgctccgcccctctgccctgcccctgcccttgctgcacacagccctccaggggccggctgagagtggagcagaaagggcccccggagctgagcgaggaggacaaggcagcctccacctgcgctaggtaggcgtcctgctctcgagactttgagttcctgtggagggtggggggtgtcatcctttta

SEQ ID NO: 74 - DNA, 2145 bp, Mus musculus
caaaacccagacccccaggtcccggccaatggaggggatttagactggaccgcgtctgtcaaaagctcgactccgcaccaccgcggagtccagaagaagagctggagccaccgcactccctccccgcccccgccgggatttattgagtatttggactggacaattaagtggccctgatgatgttaccaagcccggtcacctccacccctttctcagtcaaagacattttgaatctggagcagcagcggcacttccacggagctcacttgcaggcggaattggagcagcacttccactcggcaccctgcatgctggccacggctgaagggacgcagttttccgatgcaggggaggaggacgaggaagaagagggagagaaactgtcctatttgaactcactagctgctgccgagggccatggagattcgggtctctgtcctcagagctatgtccatacagttctccgagacgcttgcagcgggcccaaggaacaagaagaggaggttgtgagcgaacggagccaaaaaagctgtcagttgaagaagtctctggaagcggcgggagactgtaagacgagcgaggacggcgagaggccgaagccgcggagccgccggaagccccgggtgctcttctcgcaagctcaggtctttgagctggagcgcaggttcaagcagcagcggtacctgtcggcgcccgagcgcgagcatctcgccagcagcctgaagctcacgtccacgcaggtgaaaatctggttccagaatcgcaggtacaagtgcaagagacagcggcaggataagtccctggagctggggacgcacgcgccgccgccgccaccccgccgcgtggcagtgccggtgctcgtgcgggacggcaagccgtgcgtcacgcccagcgcacaaacctacggctcgccctacggcgtgggcgccggcgcctactcctacaacagcttccccgcctacggctacgggaactcggccgccgcagccgctgcagccgccgcagccgcagcagcagcggcggcttacagcggcagctacggctgcgcctatccgaccggtggcggcggtggtggtggcggcacggcctccgcggcgaccaccgccatgcaacccgcctgcagcgccaccggcggtggatcctttgtgaacgtgagcaacctgggaggcttcggcagcggcggcggcgcgcaacccttgcatcaaggtgctgcagccgggtccgcgtgcacgcaggggactttgcagggcatcagggcttggtaaggcctgggcgggacacgcggcgggcgtcctactgcacgctgggcaccgcgggaatgagacacgagaaaggacagacccaagggccaggtcccctcgttaaaataaaaatacagacgtatcgctccttccaaggggccatggtcgcagctcactccaggcctggggcatggaactcagcggagaaaggatgccctggtccagtcctcaagactgagacagaaacaccggccaggcgccagggaagaggtggcctcccatcctgactctgcggagagtgcgggaggctgggactccggctgtcgcttgctctccggaggaggaggagggcttttctcttcctgctttcccttggccttctccgagtgtggtccgggaaccggaccgaacaaggggggaggggggagaaagaaagaaagaaagaaaaagaacaatttaaagaaaaagaaaggtagttggagagagaacgtgaaggagaggaaaatggggtactagcttgagaaattccaccgatccgcagccccttttgcttgcccggagcgccacaacgaagtggatttttccttttccaactgtgtcccttccaagacagactccgcccggatccctagattgtcaggggactgttcttggagaccaccaccgcagtgagatcgaaagttgggggaagggaggcagctgagcaaatccaagacctgtccgcccaacctaggttgtcgccaggttggtttcctttctgttttgtattctgtattccgcacattatctctctataactatatccacacgccgtgtacacagccgctgtcacacactacaggagtcaataaagaaggtgcaatatttctgaaaaaaaaaaaaaaaaaaaaaaaaaaaaaaaaaa

SEQ ID NO: 75 - DNA, 4087 bp, Mus musculus
tagacggcgagaggacgaccctggccaaggcagctatggaaaccaaaggctaccacagtctccctgaaggcctagatatggaaaggcgctggagtcaagtgtctcagaccttggagcgttcttctcttggacctgcagagaggaccaatgagaacagctacatggagattgtcaacgtcagctgcgtttccggtgctactccgaacaacagtactcaagggagcagcaaagaaaaacacgaattactcccttgtcttcagcaagacaatagtcggtctgggattttgccatcagatattaaaactgagctggaatccaaggaactttcagccacggtggctgagtccatgggtttatacatggattctgtgagagatgccgagtacacttatgatcagcaaaaccaacaaggaagcctgagcccggcaaagatttatcaaaacatggagcagctggtgaagttttacaaagagaatggtcacaggtcctccacactgagtgctataagcaggcctttgaggtcattcatgcctgactctgggacctccatgaatggtggggccttgcgtgccatcgttaagagcccaatcatctgtcatgagaagagcccctctgtttgcagcccgctcaacatgccgtcttcagtatgcagccccgcgggcatcaactccatgtcctcctccacagctagctttggcagtttcccagtgcacagtcccatcactcaaggaacctcactgacatgctcccccagtgttgaaaatagaggctcaaggtcacacagccccgtacatgcgagcaatgtgggctctcctctttcaagtccattaagcagcatgaaatccccaatttccagccctccaagtcactgcagtgtaaaatctccagtatccagtccaaacaatgtccctctgcgctcctctgtgtcaagcccggcaaatcttaacaattcaaggtgctctgtttccagcccttccaacaccaacaatagatctacactctccagcccgacagctagcacagtggggtccattggcagccccatcagcaatgccttcagctataccacttcaggcgcttcggctggagccggtgccatccaggatatggttcccagtccagacacccacgagaaaggtgctcacgacgttcctttccctaagacagaggaagtcgagaaggccatttccaatggtgtgactggtcagctcaacattgtccagtacataaaaccagaaccagatggggctttcagcagttcctgcctaggaggaaacaacaaaatcaaccccagttctccgttctctgtaccaataaagcaagagtcaagcaagcactcatgttcaggcgcctcttttaaagggaaccccacagtcaacccatttccattcatggatggctcgtacttttcttttatggatgataaagactattattccctatcaggaatcttaggaccacctgtgcccggctttgatagtagctgtgaaggcagtgcgttcccgggggggattaagcaagaaccagatgatgggagctatttccctgaaaccagcatcccatcatctgccatcattggtgtgaattcaggtggacagtcctttcactaccggattggtgctcaaggtacaatatctttatcacggtcacctagagaccaatctttccaacacttgagttcctttccgcctgtcaatgcattagtggagtcatggaaaccacacggtgacctgtcatctaggagaagtgatgggtacccggtcctagagtacattccagaaaacgtgtcaagctctactttacgaagtgtttctactggatcctcaagaccttccaagatctgcttggtgtgtggagatgaggcttctgggtgtcactatggggtagtgacctgcggcagctgcaaagtcttcttcaaaagagccgtggaaggacaacacaactatctgtgtgctggaagaaatgactgcattattgataagattcggagaaagaactgtcctgcctgcaggctccagaaatgcctccaagccggcatgaacttaggagctcgaaagtcaaagaagctggggaagttaaaaggccttcacgaggagcagccacagcagcccccaccgccaccaccccagagcccagaagaggggaccacatacattgctcctaccaaggagccatcagtgaactctgcgctggtcccgcagctcgcctcgatcacgcgtgcgctcacgccatccccgtccatgatcctggagaacatcgagcccgagatcgtgtatgcaggctacgacaattccaagcctgacaccgccgagagcctgctctccacgctcaaccgcctggcgggcaagcagatgatccaagtcgtgaagtgggccaaggtacttccaggatttaaaaacttgcctcttgaggaccaaattaccctcatccagtattcttggatgtgtctatcatcgtttgccttgagttggagatcgtacaaacatacgaacagccaatttctctattttgcaccagacctagtttttaatgaagagaagatgcatcagtctgccatgtatgagctgtgccaggggatgcggcagatcagccttcagttcgtgcggcttcagctgacctttgaggagtactccataatgaaggttttgctgctactgagcacagttccaaaagatggcctcaagagccaggctgcgtttgaggagatgaggacaaattacatcaaagaactgaggaaaatggtcaccaagtgtcccaacagttctggacagagttggcagaggttctaccaactgacgaagcttctagactccatgcatgatttggtgaatgacctgctagaattctgcttctataccttccgagaatcccaggctctgaaggtggagtttcccgccatgctggtggagatcatcagcgaccagctgccaaaggtggaatccgggaacgccaagcccctttactttcacaggaagtgatggaagatgccggccaggaagaactttgccttaagtttccccacgttgttctacaactacgaaggacccaagaaatcacatttttaacatgtgacggcggattgacagttggtcggcagtttctcaagtttaaaatcccatcgagggtgtggagtcgggaaagccaggagacctggatcaggcaagcctgagctgactaagggactcttccctctccactcccagtgcttagaaaacatgttcccgttcctctggatgaaaagccatatctagtcaataactctgattttgatattttaacagatggaagttttaactatgccatgtggtttctggtatctctcgcttgttttcaaagggttcaaaggactagagaactttttaaagcttacccttggtttgcacataaaatgtctagtcaatatggggcattaatattcttttgttatttaaaaacacacaaaaagcatacacaaaaatacatacagattcctggtatgtaataaacagaacacgtggcgtggaactgtggccttccaggcacgtttttctgcatcgctggcatacacactcgttagtgtccatttattatttaattagaatggataagatgttaaatgccttggtttcgatttctagtatctattgtgttggctttacaaataattttttgcagtcttttgctgtgctgtacattactgtatgtataaatcgtgaaggacctgaaataaggtgtaaggaacttttgtaaatgagacacaaaaaaaaatctttaatggttaataggatgaatgggaaagtatttttgaaagaattctattttgctggagactatttaagtactatctttgtctaaacaaggtaacttttttttgtaagtgagatgccctgcatgcgtactgaaccgtttacagtgtatttaagaaagggaaagctgtgcctttttttagcttcatatctaatttaccattttacagtctctgttgtaaataaccacacttaaacctctttggttgtctctaagcctttctactttttcagtactatttgttttgttcttggtctcctgcgtggggtgttcgtgagactccagcgcgtagttctggctt

SEQ ID NO: 76 - DNA, 3978 bp, Mus musculus
tcctccttctcctcctccacctcctcctcctccttctcctcctcctcctcctcctcctctcaggctgtggtttttctgtatatgtttctggagtcctgagcctgggctaaacaaaagcaggaggctgacggggctgctggagtttgcagagacacggaggaggaaagagcttgggctttgcctgccgccacggctgggggtgatctggcttctgctacagcccaccccctttgagatcactctggcccggagtgggggtggggggcagcggggggtgggggggaaagtttgcattgcaatccccctgccttcctctcctttctcccgatcaatgcatatttgcaaaaggattaagccacagatttaagcgccgggagcccatttctgccttgcaaaggagatcggactgaaaaacccaaaggcggctctgatttcttttcgccaagtgggaaggtggtttatttttcttgcttttttggagtcaacacccttccccaccagcccttatccccaccctcaccccgcaaccccttcacgcccccctccccctccccatcctcccaccatcctctaaagaggcaaagagattttttttttccccttttggtcttcttcttctttttttaaaaccctggtccctgtttatcctgaaaaggatttgaagacagcttgaaggataaaaagcctcggtgcttcccaggcgccgatccgaggagccgaagaggaagagccggggctgccggagccttcagagatggacgagcagccgaggctgatgcattcccacgctggggtcgggatggccggacaccccggcctgtcccagcacttgcaggatggggccggagggaccgagggggagggcgggaggaagcaggacatcggggacattttacagcaaattatgaccatcacagaccagagtttggatgaagcgcaggccagaaaacatgctttaaactgccacagaatgaagcctgccttgtttaatgtgttgtgtgaaatcaaagaaaaaacagttttgagtattcggggagcccaagaagaggagcccacagacccccagctcatgcgactggacaacatgctgctagcagaaggggtggcggggcctgagaagggcggaggctcggcagcggcggcggcggcagcggcagcttctgggggtgcaggttcagacaactcagtggagcattccgactacagagccaaactctcacagatcagacaaatctaccacacagagctggagaagtatgagcaggcatgcaatgaattcaccacccacgtgatgaacctccttcgagagcaaagccggaccaggcccatctctccgaaggagatcgagcggatggtgagcatcatccaccgcaagttcagctccatccagatgcagctgaaacagagcacgtgcgaggccgtcatgatcctgcgctcccggttcctggatgcgaggcggaagagacggaatttcaacaagcaagccacagaaattctgaatgaatatttctattcccatctcagcaacccttaccccagtgaggaagccaaagaggagttagccaagaagtgcggcatcacagtctcccaggtatcaaactggtttggaaataagcgaatccggtacaagaagaacataggtaaatttcaagaggaagccaatatttatgctgccaaaacggctgtcacagccaccaatgtgtcagcccatggaagccaagctaactcgccctctactcccaactcagcgggtggatacccttcgccatgttatcagccagacaggaggatacagtgacggactcgcagccagtcagatgtacagtccgcagggcatcagtgatatgtcaagaacctactgatcctcacaagaacctactgtctctcttctcttgactgaaaacaaaagtcttcttctacctgacccgtggcctgacttctggaagaatgcatatggactttcgaagaagtcagaggatatctgctggccacttgtatcagacaaaacaaggctggtgagcagggagctagcagatgttgtaaccagatgattggtacatcgacagagtcccaaagcaataaccactattctttctcttctccactaatccagcaatcaaataatcactcttgagcatttactgtgtaccacaatataatataaaatgtggtgagtagctagatatataaggtaatctctcaactctcaaaaaaagtaatgcatttacaaagtaagagttatgcatgtgacaagacaactaatcgcataggggagagaacatggttgcaggtatagcatctcccacatgtcagtcactgtgggaggggttgcacatgtattattttgactaatccctaccacatctccaagaggaattcctatcctctaataaataatgaaaattgagagtccgaaatatttagtaacttgcccatggccacatagataaaaaaaacctagagcctattttcaggcttagatgtacctaactaactccatagtctatgctttgttactaacccagacctgccagtgagagaagtcagagctcaaagacatcatttagtgcagctaagccaagacaaaactctaaaatgtttatcagcaaaatttgttttatctggcccattgattggtttggaaactagttctagccagaagataaatgcctagctactacagacagaaatgagggactataaatgaccacatggtagtctcagtgctgccaccatttatgtgtgttcatctgagcaagttgcttccctcttctaggatcttgtgccatctttggtgacatagggcatttcccccttgtctaatgtcacactctgtcactaggcatctctatggtaataacaaggtctacaataaagcaggcccaagtttgaatcctggctccatcctcaagccttgggctgttgggcaagtcatgtatacaacagagataataatactacctcagggtctctgcccttattgttcattctccttggaacagttcccagttgtccataaggtcgcctgtagagctccttccagttttactcaagttacccactagctagggctcccttggtctctaaccttgcaaatggcatttgccatttcctattttctttcttcatgctcttattttccttaacgctgaacacctaacattctatatagagcacttgattatcttatttattgcctgtgcccatcttcttacctctagaatgtaagctctctatgggcaggatttttttgtattctttactgttgtacccccagtttctagaatgaattaggtgcttaataaatatttcttgcataaatgaatgttcattgcgaggatcgtttgagtcaagggttttgcatgggatctgtcacatggtagacacatggatgttatctacgtgcctggcagttgtcgttttattattctacctaatctcaataccatgtgttctctttcccttccagatgacccaaggttaattagccttctgacttctttagtgaattttaaagtccatttctgagattattcaaataaaagaggataaagtctttgtgacagaaataaggagaaaggcttgcaaggcagagttaccttcccttcccatacacaaaggcacgtgacatgagtagaaattccagaaagcatatttctggtgcacaagaaagaattttcatagaattgaatgttaacagagggagtaagttatttcaagagagaggaagctgcttacacctggaggtctccttagaagctaggaaactgtatcttacagatctagagtacaattatagtgggtgacaaggttggagttcagccaaagggaagcactctctgagatcaatggagatggctagtaaaatgtggcatccctaggaaacaagacaaatgggtaaccaataaaagtttgttcaatgtacataatcaaataaatcaaagatagatgatcaagaaaaaaaaaaaaaaaaaaaaa

SEQ ID NO: 77 - DNA, 8433 bp, Mus musculus
gtgctctgtgcgcatgtgcgaaggtgtccaaactgacaatgctagggagatgaagatagtgtgtagctgcttctgggctcaaggaggaggagagagattccgcgagccgacaccatgcgatccaaggcgagggcgaggaagctagccaaaagtgacggtgacgttgtaaataatatgtatgaacctgacccggacctgctggccggccagagtgccgaggaggagaccgaagacggcatcctgtcccccatccccatggggccaccgtcccccttccccaccagcgaggacttcactcccaaggagggctcgccctatgaggctcctgtctacattcctgaagacattccaatcccaccagacttcgagctacgagagtcctccataccaggagctggcctggggatctgggccaagcggaagatggaaatcggggagaggtttggcccctacgtggtgacgccccgggccgcactgaaggaggccgactttggatgggagcagatgctgacggatacagaggtgtcatcccaggagagctgcatcaaaaagcagatctctgaagacttgggtagcgagaagttctgcgtggatgccaatcaggcggggtctggcagctggctcaagtacatccgtgtagcgtgttcctgtgatgaccaaaacctcgccatgtgtcagatcaacgaacagatttactataaagtcattaaggacatcgagcctggagaggaactgttggtgcatgtgaaagaaggtgcctactccttgggtgtcatggcccccagcttggatgaggaccccacattccgctgtgatgagtgtgatgagctcttccagtgcaggctggacctgaggcgccacaagaagtacgcgtgcagctctgcaggagcccagctctacgagggcctaggggaggaactcaagcccgagggccttggcgtgggcagcgacgggcaagcgcatgagtgcaaggattgcgagcggatgttccccaacaagtacagcttggagcaacacatgatcgtccacacggaagagcgtgagtacaaatgtgaccagtgtcccaaggccttcaactggaagtccaacctcatccgccaccagatgtctcacgacagtggcaagcgcttcgaatgtgaaaactgtgtcaaggtgttcacggaccccagcaacctccagcgtcacatccgctcacagcatgtcggtgcccgggcccatgcctgccctgactgtggcaagaccttcgccacatcctctggcctcaaacagcacaagcatatccacagcacggtgaagccattcatatgcgaggtctgccacaagtcctacacgcagttctccaacctgtgccggcacaagcggatgcacgccgactgcaggacgcagatcaagtgcaaggactgtgggcagatgttcagcactacctcctccctcaacaagcatcggagattctgcgagggcaagaaccattacacgcctggcagcatcttcaccccaggcctgcccttgacccccagccccatgatggacaagacaaaaccctccccgaccctcaaccacgggggcctaggcttcagcgagtacttcccctccagacctcatcctgggagcctgcccttctcggctgctcctccggccttccccgcactcactccgggcttcccgggcatctttcctccatccctgtacccacgaccacctctgctacctcccacgccgctgctcaagagccccctgaaccacgcgcaggacgccaagctacccagcccgctgggaaacccagccctgccccttgtctccgcggtcagcaatagcagccagggtgccacagcggccaccgggtcagaggagaaatttgatggccgcttggaagacgcatatgcggagaaggtcaaaaataggagccctgacatgtcggatggcagtgactttgaggatatcaacaccacgaccgggacagacttggacactaccacgggcacggggtcagacctggacagcgacctggacagtgacagagacaaaggcaaggacaaggggaagccagtggagagcaaacctgagtttgggggtgcatctgtgccccctggggccatgaacagtgtggccgaggtaccggccttctactcacagcattccttcttcccgccacccgaggaacagctgctgacggcctcgggagctgccggcgactccatcaaggccatcgcgtccatcgcggagaaatacttcggtcctggcttcatgagcatgcaggagaagaagctgggctcactaccctaccactccgtgttccccttccagttcctgcctaactttccccactccctctacccctttacggaccgagccctcgcccacaacttgctggtcaaggctgagccaaagtcaccccgggatgccctcaaggtgggcggccccagtgcggagtgccccttcgacctcaccaccaaaccaaaagaggccaaacccgccctgctcgcacccaaggtccccctcatcccctcatctggcgaggaacagccactggacctgagcatcggcagcagggccagggcaagccagaacggaggtggccgtgagccgcggaagaaccacgtctacggtgaacggaagccgggggtcagcgaggggctgcctaaggtgtgcccagcacagctgccccagcagccctccttgcattatgctaagccttcaccgttcttcatggatcccatctacagggtagaaaagcggaaggtggcagaccctgtgggagtcctgaaagagaagtacctgcggccgtccccacttctgttccacccccagatgtcagccatagaaaccatgacggagaagctggagagctttgcagccatgaaggccgactcaggcagctccctgcagcccctgcctcaccacccgttcaacttccgctccccacccccaacgctctcggatcccatcctcaggaaggggaaggagagatacacgtgcaggtactgtggcaagatcttccccagatctgcaaatctcacaagacatctgaggacacacacaggggagcagccatacaggtgcaagtactgtgaccggtcattcagcatctcctccaacctccagcggcacgtgaggaacatccacaacaaagagaagccgttcaagtgccatctgtgcaaccgctgcttcgggcagcagaccaacctagaccggcacctgaagaagcacgaacacgagggcgcaccagtgagccagcactccggggtgctcacgaaccacctgggcaccagcgcctcctcccccacctccgagtcggacaaccatgcacttttagatgagaaggaagattcttacttctccgagatccgaaacttcatcgccaacagcgagatgaaccaggcatccactcgaatggacaaacggcctgagatccaagacctggacagcaacccaccgtgtccaggctcagccagtgcaaagccagaggacgtagaggaggaggaagaggaggagctggaggaagaggatgatgacagcttagccgggaagtcacaggaggacacggtgtcccccacacctgagccccaaggagtctatgaagatgaagaggatgaggaaccacccagcctgaccatgggctttgaccatacccggaggcatatgcaatgatgctgtccctctctgaagacactcctctccacgccccctcccagagctcactggatgcttggttgaacatcacaggaccctcgtcagagtccggagcctttaaccccatcaaccacctctgaggtcctggaggccccagggccagagtccaaagccagggtaccagctgcaggagatggagaaggggccagggagcagccccccaccctcaacacctccactttgcaaagtccagcttctccattgaaactcagaacccgaaggtcccttgagtagccgctggccttcatcacctctcagaactggcctcaaggacacgatctgcagtgggtgcggtgcacgggccacccaggagctgctcacaggagccatggatcagaaaactcgtgggcaagggtggggtctctatcccagcaggagccagttggccacatccaggcaactgcatggtatgaaagaggaaatcggaaagacgtgggcaagtgctatggagagagacctcatcaatgatttttataatgagaatcacatgattaagccttttggtaatcttattgactatagagtctatttaagcatgtgggttttaaaaaaaatagacggtattttttaaaaatcaaaaaaaaaaaaaaaagacttgcaaaatgtttttttaaaagtaattttgcatcgctttggaatttcagcttattggcaaacccacacctgccaggacatcgagtacggctcgggtgtgccctttataatgtaaataatggagaattttttttctatctctgacctgtttgtataagccaaggtgattccagtgtgcccgacacaggataaggggcatggagccactctgctaagtcagatgctgactacacaagaattcatggacgtggtgcctggctgcaggtttgcagagggaggccaggagaaagaggtcgccttgccctgtgtgctctgcagaggaatccacgggtcccacccaagaccctccccaattgtatggggaagtcgggagccagtggctcaggtcttccatgctggacacctaaggatcttccccttcagcagcagcactggccttgactgctgctcttctgaaggcagggctctgggccatgtggaggcctccagccttctaccctccccaaccctggcaggctgatgccctccccatggccactgagtgtgggacgcctgcccacctgctactccactccaggacaccaagcactacctgtgcctaagactccagatcaatcacacagtcgccacttctgctctttgtgtgggttcctggtgagaataggacggggacaggaggaagatccaggggacctgccacagcaaagaacagagacagtacagaccccaagaccggatgcctcactcatttgggacggtcagaacgtgtctacttctctgcttggatgggctttaaattatcccataggggccaatttcaaataatttcttttcctgatggaatttaccttaatctgtatataacttgtaatttttttttctaattcatttctttccttattttatttcttccttaacagtatttttggcattggacaacttattctgaagaaatgatgttaatataagtatgtagtgaaggaccaaaccgtgtgataaggttgtgtgctgtgtgatcggtacccagggagcggggcggtgttaatgtgccaaatacccctgtgtccctccctccaggagttccacggtcaccactgcccacttaccagacaatcatatgattttgttaaatttgagtttcagttacattttaagacaagtgttctatttattttattgtttggaaggaggggggcatatcccaacaagataagaaggaacagagaccgcctgggttgtcatttaaaactacaagagattgtgattgtgtgagcgtcagcaaactcggagcacgcgtgtcatgccgtgggcctgtcagcggttccctctgggagcaacttaaacatccaagcacagatcttttatatttgctaaggccgttctgggacaaagaccgtcatctttgctgatgctcagtggtccaggctacgctgggatccgtgatggggatatcccagaagacaccattcccctgcaggttgcggtgaatctcagtgacatagcatggaacagtcagtcctgtgccgtgctcagaagcacactctcgagtgcacactggacggcctgaggcagcagggacactctgcagaggaacttgccatggtccagtggtaaggattcgtaggtctaactgctcatgcttctcatgggttcgtgacatcatctccatggcgggccaggcctcggcttgagaatcccagtttcctctaggtgggagctagtcctgtgtaagttcaacctcagatatacatgccccacttttgtaaaccccaaattatatgctttcttctcagaaagagcaaggtgtgtgctttttttttcccccgcggtatgactttctctctccggtacggggagtctatctggatggggatcttctgtggacctttttgacagcagcagagcagctaaaggctcccttctcatcctctggcacgcacaacccctgtccatctctaacccaagagtcccatctgtgctgagttaaatgctttgcccccacaaagtgatgattccaaactcaggttatcctgaacagtcatccccttgggcccagcacttggaatttgaaggactccgtccttgcttgccacccccgacaaagacagcacttgattttctgcactgagttccagatattctgcctctactctgcattgccctcaaccagtgagctctgcccacctggcgtctctgcagtcttaatagtgcccagctggctcaagcaagcatcagatcaggtacaggagaccctctcagagtcaggaagctggaccaggtgtgagttcaaaggtcttggccactggagaaccagaccattaacttggctctcttgcctgttgctgtgcaggatagaggggcagggcagaggttcagccacgattgtttcccaatgatactttccattgtttgcacttgcctcctcaaatagaaattcattcatcgggaggccacctagcttgcattccctctagccccgggcaaggccacacttaggccctgtcattgctgccacacctaggatgatcaggtaggaaacgcagggcttcactgtcctgcaggaaggtagatcctgcatgagtaagaccccgtacatccctgtgcaaacccagggtgtaaacctcagtaatgggagagcatgacatggccaagcctgtggggccctactctccaaggccggggagagcaggtaatctacccagacccagactgacctgacccctctgcctcccagggtccagacccagagctcttggcttcctctccctcatctgcctccacagtgagtgtctgcgaagcacttgcttaaatacatatcacgtgttttagactcgaagctcaaagcactggattgtggtccctgtccctgcccaagtggctgagacctactgagctgttcactgtgccgttcgaggtagggaagaaaaggcctccgagctgttgctgggacaggagggtggagaggcctgggacagacagcgctccctcctgccagtagacactgggacatccctgtccacggagatcagccgggtatcgaaagccatgaagactgtccctagccctgagcgatactgtgacaagacattcttgaattctccccagacaaggtcacttcccccaccattccagccattgtctgtctgtcaccaattcacatcctcacttctaggagggggaaaggttgagtctttagatgtgcgattgatccccttgcaactgggggaagctgggggtttcctcagtgtctcccacagcgcagctactctcttcagtgtggggaaaggggggccctggtgtcagaatacgtaacttaaagcctgtgcgtgtggtggtgcgtggtgtgtgtgtgtgtgcatgtgtgctgtatgtgtgtactttgaaccaatagtatataaatataaatataaatatatatatatatccctgacacccgcgagaatctccaccctgtatgagagttgccattgttacattcagatcggattcagttcgtttctgtttctgtttttgttgttgttgggtttttttttttttccttttagaactatatagtgttgagaaaggaatcaaatgtaaatgtctggtttccatataacgttaaaaaagaaaattaaaaaaccattaaggagggggctggtgtctgccctgcccccttccctgtaaattgctgttgtgttgataaagctgtgcatggctcgtgtttagcggtcattattgtgtctgtttgtggaattcttatgatgaggaaaatcccgtagatgcacttattgaatatgtgattaggtctaagtcagggccaggagtcccaaaacgcccacaagaaaaagataaaaagaaaaaaaaatggaccacagttcctggtgactatggaaatcgccgtgagagaatcgtgaatcgagaagacgcggcttgagaatagtcaaatgtgattttttttttttttttttttttttgcagatctcattctgaggcttatggcaaagacaaatgtacttgtgtagaggtttctgtgaggtaaaagccaggagggcctggctttgaggtcaatgctgtagaataggactgactggataccaaatgtaatctttccgatgctacaatgaatttatacacgagattgatatgcaataaatctgtgtgtttttc

SEQ ID NO: 78 - DNA, 2182 bp, Mus musculus
gaggaggggcctggccgcggggagccgaggagggaggtggggattctgggagccgagctcatttcctcctcccggccgccccaggcccgcaccccatctccacgtgcggcgcctccggagcgcggccgagccacggaggatgctgggcatgtacgtaccagacaggttcgccctgaagtcgtcccgggtccaggacgggatggggctctacacggcccgccgcgtgcgcaagggtgaaaaatttggacccttcgctggggagaagcgaatgcctgaagacttggatgaaaatatggactacagactgatgtgggaggtacgtgggagcaagggagaagttctgtatattttggatgctaccaacccaagacactccaactggcttcgctttgttcacgaggcaccatctcaggagcggaagaacctggctgccattcaagaaggagaaaatattttctacttggcagttgatgatatagaaacagatacagagcttttgattggctacctggacagtgatgtggaggcagaggaggaggagcaacaagctctgaccatgaccaaagaaggcaaagttgaccactctaagggacagttggcagctggaagtaaaggtcaccttggctgtgaagaggactttgcctgtccacagtgtgaatcgagctttcccagtgaggaagtccttactgagcaccttcagagcttgcaccagaagcccacaggggagaaagagttcaaatgcgagaactgcgggaagaaattccctgtgaggcaggccttgcagagacattttgagcagcaccggaaggcttgccgaggggaggccaggtttgtgtgcaaagccgacagctgcgggaagaggctgaaaagcaaggatgccctgcgaaggcaccaggaaaatgtccacaccggtgatcctaagagaaaactcatatgctcggtgtgcaatagaaaatgtacctcagtgtcaagcctgcaggagcacaggaagattcatgagatatttgattgtcaagaatgtatgaaaaagtttatttctgctaatcagctgaagcgtcacatgattacccactcagaaaagcggccttataactgtgagatctgtaacaagtccttcaagaggctcgatcaagtgggcgcccacaaagtgatccacagtgaggacaaaccctaccagtgcaagctctgtggcaagggctttgctcacagaaacgtttacaagaaccacaagaagacccactccgaggagagacctttccagtgtgatgcatgtaaagccttgttccgcacgcccttttctctgcagagacacctgttaatccacaacagtgagaggacttttaagtgtcaccactgtgatgccacatttaaaaggaaggatacattaaacgttcatgtccaggtggtccatgaaagacacaagaagtaccgatgtgagctgtgcaataaggcctttgtcacaccttcagtgcttaggagtcataagaagacacacacaggagaaaaggagaaagtctgcccatattgtggccagaaatttgccagcagtgggaccctgagagttcacatccggagccacacaggtgagcgcccctatcaatgcccgtactgtgaaaaaggtttcagtaaaaatgacggactgaagatgcacattcgtactcacaccagggagaagccctaccagtgctcagagtgcagcaaggccttcagccagaagcggggcctcgatgaacacaagaggacacacacaggagaaaagccttttcagtgtgacgtatgtgacttggcttttagcctgaagaaaatgcttattcgacacaagatgacacacaatcctaaccgtccgatggcagagtgccatttctgccataagaagtttacaagaaatgactacctcaaagtgcacatggacaacatccatggggtagctgacagctaagaggagcggcaaggaaccacaccatgtgaaagagcttctactatgaatcccagattcttctcacctgatcggcttaacagaaatagccacaaaggattcattgatctgacagtgtttatgtgcctatctttgtaatctatagatgcaaaaaaaatccttttaccaaaaataaattcaaaatagaaaacaataatactttgtagattacagagtattctggctgattaaaaattaaatacagag

SEQ ID NO: 79 - DNA, 2789 bp, Mus musculus
taaatgatcatttggatcaattacaggcttttagctggcttgtctgtcataattcatgattcggggctgggaaaaagaccaacagcctacgtgccaaaaaaggggcagagtttgatggagttcgtggacttttctgtgcggctcgcctccacacctagaggataagcacttttgcagagcgcggtgcggagagatcatgtttgactgtatggatgttctgtcagtgagtcccgggcagatcctggatttctacaccgcgagcccttcctcctgcatgctgcaggaaaaggctctcaaagcctgcctcagtggattcacccaggccgaatggcagcaccggcatactgctcaatccatcgagacacagagtaccagctctgaggagctcgtcccgagcccaccatctccacttcctcctcctcgggtgtacaagccctgcttcgtttgccaggacaagtcatcgggctaccactatggcgtcagtgcctgcgaggggtgcaagggctttttccgcagaagtattcagaagaacatgatctacacttgccatcgagataagaactgcgtcattaacaaggtcactaggaaccgatgccagtactgccgcctgcagaagtgctttgaagtgggcatgtccaaagagtctgttaggaatgacaggaacaagaaaaagaaggagccttcaaagcaggaatgcacagagagctatgagatgacagcggagctagacgacctcactgagaagatccggaaagcccaccaggaaacctttccctcactctgccagctgggtaaatacaccacgaattccagcgctgaccaccgggtccgattggacttgggcctctgggacaaattcagtgagctggccaccaagtgcattattaagatcgtggagttcgccaagcgtctgccgggcttcacaggtctgaccatcgcagaccagatcaccctgctcaaagccgcctgcttggatatcttgattctcagaatttgtaccaggtataccccagagcaagacaccatgactttctctgatggccttacactaaatcgaactcagatgcacaatgctggcttcggtcctctgactgaccttgtgttcacctttgccaaccagctcctgcctttggaaatggatgacacagaaacaggccttctcagtgccatctgtttaatctgtggagaccgccaggaccttgaggaaccaacaaaagtagacaagctccaagaaccactgctggaagcactaaagatttacattagaaaacgacgacccagcaagcctcacatgtttccaaagatcttaatgaaaatcacagatctccgcagcatcagcgcgaaaggtgccgaacgtgtaattaccttgaaaatggaaattcctggatcaatgccacctctcattcaggaaatgctggagaattctgaaggacatgaacccttgaccccaagttcaagtgggaatatagcagagcacagtcccagcgtgtcccccagctcagtggagaacagtggagtcagtcagtcaccactgctgcagtgagacatttccagctgttgcagacattccccaggaccttcagttccagattgaaaatgcaaggaaaacatttttactgctgcttagtttttgaactgaaatatgttaaactcaaaaaggaccaagaagttttcatatgtatcaatatatattccttactgtataacttccctagaaatacaaacttttcaaattctgaaaatcagccatttcatgccaccagaatctagtttaaagcttctactttcctctctgaatagtcaagatgcatggcaaagacccagttgagatgatttagccctggttaagtttctgaagactttgtacatatagaagtacggctctgttctttctatactgtatgttggtgctttcttttgtcttgcatactcaataaccaagacaccgaggttgtggagtaaaccactgtacatccactgtacgtcctgctttcatgaataaccaggcctcacggtaaggaaccagggcctctgtacagtacaagatgacactaaagacactctggtttaagtagtgtggaagcttctccttgctttttgatgctctcaagcttcattctttcccttatgttgcccagatagagtacaccacttcactgcactagcagaattctgtatcactgaaactgccagttcagttaatccaacatcattgttcaattgttaatgatgtcactttaaattaaaagtggtttgttccttaatgacacaactaccgaatgaaaaaaaaaatgaagcatttttacagtcatgatagcctccaaggcaaaaacactgtccagtgttaataagtttgtttacctgttcacaagccattggagaaatatcacaggataatcagcaagttagtctgccgtctggactctagtagcccagtgtccttgctgactcaggcctgatcctgggatttcccccagtcttgatgcttgaaggtatgggcaagtggcctcctctggccttgttcaatcaccatgatgcagactgacagctctgggagaatgagtggttgacagactcaagtgtcggcttctgagttctcatgtaagcactagtggaatttttgtttgttcgtttttgatatattagcaaaagtctgtgatgtaccactagctctgtttgtacattgagatggtttaacagtgctttctatgttcatatactgtttacctttttccatggagtctcctggcaaagaataaatatatttattttaaaaaaaaaaaaaaaaa

SEQ ID NO: 80 - DNA, 6199 bp, Mus musculus
gcggccgctatggacccctgaccccgcggggtcattcggactctaacgtgtggactgaccgctactgactgcaccgcctgcccccgtctcctgccggcccttagcatgagcgagtgggacccagccgggtgacattgtgccggttggcggatcctcgattgccccctttcctgtcttgctcctccctcatgaagcgattctgagtacggggggttccggattattgttctcacggacccccgcttgtggttgggggggtatttaatctgaggccttagggtccttcgttgtcttttgagtgttttgtgtgtgtacatattttgctcttaagtttataaatatacatatattgagagtgtccacgtctcctcgctgaaccttaggaatcttttgccacaatgtcctgtgtgcactataaattttcctctaaactcaactacgacaccgtcacttttgatgggctccatatctccctctgcgatttaaagaagcagattatggggagagaaaagctgaaagctgccgatagcgatctgcagatcaccaacgcacagacgaaagaagaatacactgatgacaatgcgctcattcctaagaactcatctgtgattgtcaggaggattcctattggaggtgtcaagtctacaagcaagacatatgttataagtcgaactgaaccagtgatgggaactacaaaagcaattgatgacgcttctgcatctatttctctggcccagcttacaaagactgccaatctggctgaagccaatgcttctgaagaagacaaaattaaagcaatgatgtcacagtctggccatgaatacgacccaatcaattacatgaagaaaactctagtaggtccaccacctccatcttacacctgctttcgttgtggtaaacctggtcattatattaagaattgcccaacaaatggggataagaactttgaatctggtcctaggatcaaaaagagcactggaattcctagaagttttatgatggaagtgaaagatcctaacatgaaaggtgcaatgcttacaaacactggaaaatatgcaataccaactatagatgcagaggcctatgcaatcgggaagaaagagaaaccacccttcttaccagaggagccatcatcatcttcagaagaagatgatcctatcccagacgagctcttgtgcctcatctgcaaagacatcatgactgatgctgtggtcattccctgctgtggaaacagttactgtgatgaatgtataagaacagcactcttggagtcagatgaacatacatgtccaacatgtcaccaaaatgatgtttctcctgatgctttaattgccaacaagtttttacgacaggctgttaataactttaaaaatgaaactggctatacaaaacgactacgaaaacagttacctccacccccacccccagtaccaccaccaagaccactcatgcagcggaacctgcagcctctgatgagatctcccatatcaagacagcaggatcctctgatgattccagtgacatcgtcctccgctcactcagctccctctatatcttcattaacttcaaatccatctgccctggctccttctgtgtctggaaatccatcttctgccccagctccagtacctgatataactgcaaccgtgtctatatcagtccactcagaaaaatcggatggaccttttcgggattctgataataaattattgccagctgccgcccttacatcagaacattcaaagggagcctcttcaattgctattactgctcttatggaagaaaaggggtaccaggtaccagtccttggaactccatctttgttgggacagtcattattacatggacagttgattcccacaactggcccagtaagaatcaatgctgctcgtccaggtggtggccggccaggctgggagcattccaacaagcttgggtacctagtttctccaccacagcaaattagaagaggagaaagaagctgttacagaagtataaaccgcgggcgacaccacagcgaacgatcacagaggactcaaggcccatcacttccagcaactccagtctttgtgcccgttccaccacctcctttgtatccgcctcctccccatacacttcctcttcctccaggtgtacctcccccacagttttctcctcagtttcctcctggccagcctccaccagcaggatatagtgtccctcctccagggtttccaccagctcctgccaatatatcaacaccttgggtatcatcaggagtgcagactgcccattcaaataccatccctacaacacaagcacctcctttgtccagggaagaattctatagagagcaacgacggctaaaggaagaggaaaagaaaaagtccaagctagatgagtttacaaatgattttgctaaggaattgatggaatacaaaaagattcaaaaggagcgtaggcgctcattttccaggtctaaatctccctatagtgggtcatcgtattcaagaagttcatacacttattcaaagtcaaggtctggctcaacacgttcacgctcttactctcggtccttcagccgctcacattctcgctcctattcacgatcacccccataccccaggagaggcagaggcaagagccgcaattaccgttcacggtccagatctcacggataccaccgatctaggtcaaggtcacctccctatagacgataccactcacggtccagatctcctcaagcatttaggggacagtctcccactaaacgcaatgtacctcaaggagaaacagagcgtgagtattttaatagatacagagaagttccacccccttatgacatcaaagcctattatgggcggagtgtcgactttagagacccatttgagaaagaacgctaccgggaatgggaaaggaaataccgagagtggtatgagaagtactacaaagggtacgcggtgggagctcaacctagaccctcagccaatagagaggacttttctccagagagactcttacctcttaatatcagaaattcaccctttacaagagggcgcagagaagactatgctgctggacaaagtcatagaaatagaaatctaggtggcaactatccagaaaagctttcaacaagggacagtcacaatgcaaaagataatccaaaatcgaaggagaaggagagtgagaatgttccaggagacggcaaagggaacaagcataagaaacacaggaaacgaagaaagggggaagagagtgagagcttcctgaacccagagctactggagacgtctaggaaatgcagggaatcgtcagggattgatgaaacgaagacagatacactgtttgttctcccaagcagagacgatgctacacctgttagggatgagccaatggacgcagaatcgatcactttcaagtcagtatctgacaaagacaagagggaaaaggataagccaaaagtaaaaagtgacaagaccaaacggaaaagtgacgggtctgctacagccaagaaagacaatgttttaaaaccttctaaaggacctcaagaaaaggtagatggagaccgtgaaaagtctcctcggtctgagccgccactcaaaaaagccaaagaggaggctacaaagattgactctgtaaaaccttcctcgtcttctcagaaggatgagaaggtcactggaacccctagaaaagcccattctaaatctgcaaaagaacaccaggaggcaaagccagccaaggacgagaaggtcaaaaaggactgttccaaagacatcaagtcagaaaagccagccagtaaggacgagaaggccaagaagcctgagaaaaataaactacttgatagcaagggagaaaaacgaaagagaaaaacggaagaaaagagtgtagataaagattttgagtcgtcttcaatgaaaatctctaaagtagaaggaacagaaatagtgaaaccatcaccaaaacggaaaatggaaggtgatgttgaaaagctggaaaggaccccagaaaaggacaagattgcatcatcaactactccagccaaaaaaatcaaactcaacagagaaactggaaaaaaaattggaaatgcagaaaatgcatctactacaaaagaaccctctgaaaaattggagtcaacatctagcaaaatcaaacaggaaaaagtcaagggaaaggccaaacggaaagtagctgggtcggaaggctccagctccacgcttgtggattacaccagtacaagttcaactggaggcagtcctgtgaggaaatctgaagaaaagacagatacaaagcgaacagtcattaaaactatggaggaatataataatgataacacagctcctgctgaagatgttataattatgatccaggttcctcagtccaaatgggataaagatgactttgagtctgaagaagaagatgttaaaaccacacaacctatacagagtgtagggaaaccatcgagtattataaaaaatgtcactactaagccatcggctacggctaagtacaccgagaaggaaagcgagcagcccgagaaactgcagaagcttcccaaggaggcgagccacgagctgatgcagcacgagctcaggagctcaaagggcagtgcgtccagtgagaagggcagagccaaggaccgggagcactcagggtcggagaaggacaaccctgacaagaggaagagcggtgcccagccagacaaggagagcactgtggaccgcctgagtgagcagggacattttaagactctctctcagtcttccaaagagaccaggacttcagagaagcacgagtctgttcgtggttcctcaaataaagacttcactcctggtagagacaagaaagtggactacgacagcagggattattccagttccaagcgaagagacgagagaggtgaattagcaaggagaaaagactctcctccccggggcaaagagtctctgtctgggcagaaaagcaagctgagggaggagagagatttacctaaaaagggggccgagtcaaaaaaaagtaattctagccccccaagagacaaaaagcctcatgatcataaagccccctacgaaactaaacgcccatgtgaagagacaaagcctgtagataaaaactctgggaaggagcgggagaagcatgctgctgaagctcgcaatgggaaagagtccagtggtggcaaactgccatgtatacctaacccgccagaccctcccatggagaaggagctggctgctgggcaggtggagaagagcgccgtcaagccgaaaccccagctgagccattcctcgaggctttcctctgacctgacccgggagacggacgaggctgcctttgaaccagattataatgagagcgacagtgagagtaatgtgtctgtgaaggaagaagaagctgttgccagtatctccaaggacttgaaagagaaaacaacagagaaagcgaaagagagcttgactgtagcaacggccagccagccaggtgcagacaggagccagagccaaagtagccccagtgttagtccaagtagaagtcatagcccttccgggagccagacccgaagccacagcagcagtgccagctcagccggaagccaggacagcaaaaagaagaagaagaagaaggagaagaaaaagcacaagaagcataaaaagcacaagaagcacaagaagcacgcaggcgccgacggcgacgtggagaagagccagaaacacaaacacaagaagaagaaggccaagaagaacaaagacaaggagaaggagaaagatgaccaaaaagtgagatctgtcactgtgtgaaggacggatgtgttaattgacttaattactaagtcatctgtattaaattctgttataatgtaaagagattccagccttgtaaataatgaatggaagaccctgtgctgcacttaaaagtatttgctgcttgattatttcatttttacatcagagctttataacgaacttttgtacagaattgtgagttgtgaccatggaacatgagaggttttgctagggcctattatttttaaccaccattaattagttggggtggagtttactgtactgtgaaattttcacatttgaatttttttaattgcctggcaaatgctgatatcagttcaaaaatatcagcagaatgattgctgaactcattacagccccgttatgtcactttttgattacaataaaagttttcagtaaacttttcacttgttgcaaatgta

SEQ ID NO: 81 - DNA, 2547 bp, Mus musculus
cgagagaaagcgcgctgcgtgcggccccgctgcccagagcctgccagcagcgcgtcctcggcggctccggtcccaaccgcgcccgccgcctcgcctcctcggtctcggccgcgtcccgactccggggctcgcccggcctctcggaggtccccgcccgccggtccgtctcggctctccgctgcgccgcccggctccagccccgcagcccgccccgggggaaggtagcagcgggcgagcgcgccctcgccctcgcctccaaccccgcgccccagtcccgcgcggcgacgaaggaccgggaagatgaacggcggcggcaaagccgagaaggagaacaccccgagcgaggccaaccttcaggaggaggaggtccggaccctatttgtcagcggtctgcctctggacatcaagccccgagagctgtacctgctcttcagaccattcaagggctatgaaggttctctcataaagctcacatctaaacagcccgtaggctttgtcagttttgacagtcgctcagaagcagaggccgcaaagaacgctttgaacggcatccgcttcgatcctgaaatcccgcaaacgctacgactagagtttgctaaggcaaacacgaagatggccaagaacaaactcgtagggactccaaaccccagtactcctctgcccaacactgtacctcagttcattgccagggagccatatgagctcacagtacctgcactttaccccagtagccctgaagtttgggccccgtaccctctgtacccagcggagttagcgcctgctcttcctcctcctgccgctttcacctaccccgcttcactgcatgcccagatgcgctggatccctccctccgaggccacttcccagggctggaagtcccggcagttctgctgaggctgtgtctcgggtgtgtgatggcggctgcaatctgtcttgtgggtattaatgcggtcttcactggtggcgactgtcatccagctgttctgcaaaactggagcttgctggcttgaaaacaccctgccccgttttgaccccttcaagacttcacaacagcctctatcacacatctgttttcctcaaagaaaaaaatatatataataaaatgtgttttgctcttttacactgtataattttaagaaatgtgttatttgtgaatgcatggtctgacatttctgtacagtttgaagacatagaacaaagacagcaacagtaaagccaagatcgttagtatttttataaagacattttaagaatggacagatggattcttacactaggttatatcacgacttttcgcttgagttttgaatgcttttaatggtgtttatcttatttttcttctgaaataagatgcatgtttgaagcatatctctagcatcaaaattttccacaattgtgttgtaaatgtgtggactccctctgcctggcccagtgtcaggccctgttattagtaacgcggtgtagaaatgagcttctgcagtactagctctgtaatcctggctcccagggatctctctgcaagacaatcagtgcccccaagagcatttgccattgtagtatattcaccactgggcaaagactacttaggtaatgtctctgaaacccatggccactccagctgagactataccaactgtgcccagggacatagtgacattctatacttagcatttatgatagcaccctcagagtgtgtaacctaatatatatcacccaccttccataaactgtctccatttccccagtggaaccttatatttaaggggagggaccttagtttggaggtaaccattgcctcaagaggagcaggccctgcccactcagcctgcagtggacatcacatctctctcaagtgattcagcaaagcattggcctacctgagtagctagctggtctggcagccatgtcagatagaccgcccatcatgttctctggacgtgctcatcttgagactacctactacattgcacagggcagggaatgaaacagtggccctctgcaatgaatacccttgcttcaagacttcaaggcccaaagttagtcttgtgttatgtgcaacacatttccagaggctgtgacagcacagcctctcctgctcctcaaaaaagagctatgtattattatgttgacctcagaattcccagctgctgttactagacataatcacaatctttgtatctttgtttttgatctgcttgtgttgtgttgaaaagggtcatattttaatgatttttgtcagtggtttgggagggttatttcattatttttcatgagaagtaatgtcaagtttttaaaggtttaaaaaaagaaacaccaaggcctggaatatgggccttttcactgtaaactagaggacgggggcaggcagcactgggaacttagacatccaagctggtgttcagggactgctcatggccccagcccccgtgccacttgactgtgagactcctacttgctttatcatcaattatgcttttataaattgtgtaaaggtacttttgtattgtcattttttaaaaaaaataataaaagtttattccagccatt

SEQ ID NO: 82 - DNA, 5795 bp, Mus musculus
ttctttggacctcataaacaaccacagaaccacaagttgggtagcctggcagtgtcagaagtgtaagcccagcacagtggtcagcaggcaggacgaatcacactgaatgcaaaccacaggctttcgcagagcggtgaaagaaattatagaatcccccgccttcaggagaggtgcgttttcgaaaggaaacgatggcttcagacagcatttttgagtcatttccttcatatccacagtgcttcatgagagatgccagcacgagccgccgcttcacgccgccttccaccgcgctgagccccggcaagatgagcgaggcgctgccgctgggcgccccggatggcggcgccgccctggccagcaagctgaggagcggcgaccgcagcatggtggaggtactagctgaccaccctggcgagctagtgcgcaccgacagccccaacttcctctgctccgtgctacccactcactggcgctgcaacaagaccctgcccatcgctttcaaggtggtggcactgggggacgtcccggatggcactctggtcaccgtcatggcaggcaacgatgaaaactactcggcagaactgagaaatgctaccgcggccatgaagaaccaggtagcgagattcaacgacctcaggtttgtcgggcggagcggtagaggcaagagcttcactctgaccatcaccgtctttacaaatccgccacaagttgccacctaccatagagccatcaaaatcacagtggacggcccccgagaaccccgaagacatcggcagaaactagatgatcagaccaagcccgggagtttgtccttttccgagcggctcagtgaattggagcagctgcggcgcacggccatgagggtcagcccgcaccacccagcccccacgcccaaccctcgggcctccttgaaccactccactgcctttaaccctcagcctcaaagtcagatgcaggatgccaggcagatccagccatccccaccgtggtcctatgaccagtcctaccagtacctgggatccatcacctcttcctctgtccacccagcgacacccatttcacccggccgtgccagcggcatgaccagcctctctgcagaactttccagtcgactctcaacggctccggacctgaccgccttcggcgacccacgccagttccctactctgccgtccatctccgacccgcgcatgcactacccaggcgccttcacctactcgccgcccgtcacgtcgggcatcggcatcggcatgtcagccatgagctcggcctctcgctaccacacctacctgccgccgccctaccccggctcatcacaggcgcaggccgggcccttccagaccggctcgccctcctaccatctatactacggcgcctcggccggttcctaccagttctccatggtgggcggagagagatcgcccccgcgcatcctgccgccctgcaccaacgcatccaccggcgccgcgctgctcaaccccagcctccccagccagagcgacgtggtggagaccgagggcagccatagcaactcgcccaccaacatgccccccgcgcgcctggaggaggccgtgtggcggccctactgagctgagcgccatcgccatcgagggactgggcctgccgtccatgcacagaccccgccaggagggcccttggaggccaccaggaagaatcccggagggaaactgtgaatgcttctgatttagcaatgctgtgaataaaagaaagattttatacccttgacttcactttttaaccacgttgtttattccaaagagtgtggaatgttttcggttcggggtggggaagacgcagcccatcctgtttggcatctatttcttatttcggagttttcttttccgcaccttatcgattgcaaaaatgcctgtttgcatctgggtggtcatttatttttaagtgtgtatagatttgagcttgcttttttttcttcctttgaccaactcaaagaaataaaattcccttctctgtaaggtttatttaacttttagactttcatgtagctgggggttttatttgtgtttggtttttgtttttatttttaaagagacagctacagctttgggtcattttttaactactgtatttccacaaagaaatccctagatatttatgtatcttgatgtttgaacatttacatatgtgttgatacttttttaattatttaaatgtacttatattaagaaagatatcaagtactacatttttctttataatagccaaagttaaatattattgcgttgaagatgtctggaaaaaaaagagatcgcttggttaactagaaatattgtttacattaaactccctttatgttattcaaacaagttggtaggtaacgcagcaatgtttttaattggattgtagacactgagggtcactccaaggtcagaagtacaaaattttctgctaggctcaacaaatagtctcatacctggctccttcccttcaaaaagagaggcaaactctgtcctgaaagggttcagagaggtgccaaggatttgctctgaagaggatttcattttggcctggagatatacttgccccaaggcctcctcattctggcatgctttatcacagagctcaaccaagtaagctgttggtcaggggtttacttacatagtatttacatagacccaaaccactgaatgtgatttttaaattgccttccattaatagtacccgttcattgatgaaaaccaaaacttgaggctgtaccccaaagatccaaatagaagagttaagaccaggtgtctttgaggcctaaaggctgagttttaagagagtgtaccccaaaagtctgaaggagccggtttccttctcccagtcttagtggaatcagtcatgggaggcagatgccacgcccacctgtgcaggatgctcctcagaagctgccccttcaccagcatcttctcccaccaggccgagcccctgacctttggggtgcatcagtgtgatagatcctggtctctgcagtccgccatggctacggttcagatgtgcatcgtgtcactgtaaatgtaatggtactgttgttacagtggaggacttggtcaaaatccagttgttctacaacgtatgaagcctaaccgctggttctgacatacatgtgctcaaaatgatctggttgtttggatttttcttttgttgttttgttttttaatgtacctcttaaattagttgaagtgatgtcaggtcaactccgaagagcgtttgaaagcaggacttcagcacagtgtttgatttttttattattattaatattattttataaatttaagcattcagattagatctttggctgcaggcagcaaaaacggctggacttatttaaaaaaaatacagcttgttttttgagttatctatatctatatctatatgttgattctttgtcttacatagagcagcagcactttggtaacctgtgataccaggttgctcttgtctggagaagagcgctagcaggattcagagaaactcagaatagatcttcatatcagccataccttcctcctccatccggtctccactcagttattccacagaacactttgacagctgtgttgtcagaaaaataaaaaaaaatttaatttctcaaaaggagtttgtttctccaacattagatgttcctcttaccataggctgccgtatctggcctgagaaaacggtagggaaggacgaaggaaagagatttctattttttcatattaattttgatatctaaagatacgctagccctcagaggagcagataatctcacacattgaattttcgccctgggcaccatgcatcaagaaggcttgtcactgtgttagagccatttagtgcttcctaaacttttatcaacataggcagtatttagtctcagagaaaaaaaaatccatcaggcacatgtagtcttggagatagattccacggggcaggtatttctctacctgagaaattgtgttcattgccttcgggtgcttccagcggtctcctcattcgctgtcttcaaggaagacccataagccaattctgagataatggagctgttgggaatactggtccagagaaagaaaaatgggataagccattcttactgcttattcaagcccctatttataattttaacacactttccattccttctggttttctcgccgtctatatcctcccaatagcccttctcacttttcttttccctcctgcaaacacacacacacacacacacacacacacacataaggcacacacacacacatcctctcccccataccaagtgtccagaacacagaaagtccagttcttctccgtttattaaagaacagggtgagtcagccattctcttgctcacgggtttttttccccaacagaacagaggcgttgccagccattttgggtctgctttctgtccagatactgcagcaaaaactcttgaggatcacaacccgttggctgagcagctgtgctgctgcccaaacgtcctgcgcagacaaacgcacgctgggaccggaaggggtgtctctccttctgcctcttttctttcatacgtttctctcgaaaggcctcaactgaggactgcaaatttctttcttgaaataactttcccccagggacattcggtcttagggattttttggttttgatgggttttgttttgttttggtttttttggttcttctcattttctttgtaggagaaggcatgagatgttgagggtctttcatacatgaaaataaatagtttgacagcaatctcagaatatattttttccttatttgaacaaagtactgttttgtttactctacagtacacctttatttggtgggtttggctgttggtcggaaatgccttccctctttcagccaggtcggtatagccaaagctgatcatttgtctgttttttcttttttttcctcagaagagttgtatccacaaactcaattgtatttatgtatgtaaatagatttcatgcttcattataaaatattgttaatgcctgtaataacttttttcacattttgtgtgtgtttctaaggactttttcttatgttcgctaaatattgtagaagaaaaatgcttctcttaacttgtttattttagattttaaaaaaaccaagctacttcttactcacttttataaacataataaacaagcatggttccaatgggttttttttaagttcaccttatgttctaggagaaatgaatgtgcaaaaaaaaaaatcaatcttcaaatgaactgttgggtatttctgttactctggatggaaaaagatcaatggaaaacaaaaaacaaaaaaacaaacttccaaattgaaatagcagtaaaacacatgtattgggggtaaaaaaaaaaaaaaaaaacacacctcagaaaatgcagttctccagcctccagctactgcccacacatgtggcaagtgttagatgtggtcgtgaggaatcccaaaatcagtgcccgggccttttccttcctggtactaacattaagaacacctcctaaggggcactgtcaggctcaggtcttaaagcacatagataagagagaatgtcccagaacgattctcagtagcccaggccagcaggtactcacggcactgcacctgctcactgcacacagtcgctgaggcagacccctctcctgggggccatgcagagccagggcagggcagccctaagctactgatgttttctttcctttgtactatggtcttttctgtaactccaatattcaaacccatgtgaatggcaaataaacaatttgacaagcacctaaaaaaaaaaaaaaaaaa

SEQ ID NO: 83 - DNA, 5862 bp, Mus musculus
ctggaactggggcaggaggaagagtggagaaagggagacaggagttgtgatactttgtacacacgtccctgtcattgttctgcctgaagagacagggctgggttcaaggccacatgtgttcctgtcatcctgcacatttctgctccaagtgcaatccggagtgtcagctctccatctgtctctgcctggcaggcgcacgcgcccagcaccctgcctcgggccatgccgccccagcccctctgatggccctcctctccaccgccccgcacattccagaacaggaggcatgagcccgcatcgcgctccctcttaacacagagttgctttcagtgtgttacacgctggattccgggatgcctgatcgtaccgagaagcactccacaatgccagactcacctgtggatgtgaagacgcagtctaggctgactcccccagcaatgccacctcctccaaccactcaaggagctccaagaaccagctcatttacgccaacgacattaacgaatggcaccagccattcacccacggccttgaatggcgctccctcaccgcctaatggctttagcaacgggccttcttcttcctcttcctcttctctggctaatcagcagctgcccccagcctgtggtgctaggcaactcagcaagctgaaacgatttcttactaccctgcagcagtttggcaatgacatttcaccagagattggagaaagagttcgcacccttgtactgggactggtgaactctactttgacaattgaagaatttcattccaaactgcaagaagctactaacttcccactgagaccttttgtcatcccgtttttgaaggccaacctgcccctgctccagcgtgaactcctccactgcgcaaggctagccaaacagaaccctgcccagtacctcgctcagcacgaacagctgcttctggatgccagcaccacctcccctgtcgactcctcagagctgcttctcgacgtgaacgaaaacgggaagaggcgaactccagacagaaccaaagaaaatggctttgacagagagcctttgcactcagaacatccaagcaagcgaccatgcactattagcccaggccagcggtacagtccaaataatggcttatcctaccagccaaatggcctgcctcaccccaccccacctccacctcagcattaccgattggatgatatggccattgcccaccactacagggactcctatcgacaccccagccacagggacctcagggacagaaatagacctatggggttacatggcacacgtcaagaagaaatgattgaccacagactaacagacagagaatgggcagaagagtggaaacatctcgatcatctgttaaactgcatcatggacatggtggagaagacaagacgatccctcactgtactaagacgatgtcaggaagcggaccgggaagaactgaattactggatacggcggtacagtgatgctgaggacttaaaaaagggtggcagcagtagcagcagccactccaggcagcagagtcctgtcaatccagacccagtcgcattagatgcacatcgggaattccttcacaggcctgcgtctggatacgtgccagaggagatctggaagaaagctgaggaagctgtgaatgaggtgaagcgacaggcaatgacggagctgcaaaaagccgtgtctgaggcagagaggaaagcccatgacatgatcaccacggagcgagccaagatggaacgcacggtggctgaagcgaagaggcaggcagcagaagatgctctagcagtcatcaaccagcaggaagactccagtgagagttgctggaactgtggccggaaagcaagtgagacgtgcagcggctgtaacacggcccgatactgtggctctttttgccagcataaagactgggagaagcatcaccacatctgtggacagaccctgcaggccccacagcagggagacacgcccgctgtcagctcctcagtcacacccagcagtggggctgggagcccaatggacacaccaccagcagccactccgaggtctaccaccccgggaaccccctctaccatagagacgacgcctcgctagacctaaactcagactgtcagaggaaagacaccacaaccaacacgaaagcaattcctcatcctcagatgctcaaagtcccttttctgtttgtttgtttatagatgaactatctatcttatttcagtacttcggcaagagagaacctaactgtatctttgaggcgatagtagaacacagaggccagtaacgggtcgtaatgacttactgtggataacaaagatatcttttctttagagaactgaaaagagaggaaagaatataacgtgaaatgctagatttgacctcctccctgttatttccaagtagctgggattttaaactagatgacctcattaaccagtgctttaccaaacagcaaaccaagagattgctaattgctgttgaaagcaaaaatgctaatattaaaagtcacagtgttctttatatacaataatggaaaaaaaaaaaagaggcaattcctcaagggcatgagcggtggatacagaagtagacattttaacaagaagatgaatggcgtctgtgggttgctaactgaactttgaagacccgctgcaaaacgcgcagatgtgcagcagattggaaggagacacagatggtcctttttttcctgtttctgaaaaagaaataataatatccaggtcaacagaatgaaaaatgaaagatgatttccagtgcggtgtatgactacagcagcaagaaaaaaaaatgccataatacaaaggctcctttttatatatatagtcacacacacacacacacacacacacacacacacacacacacacttaagagactcagcctgcagttaattagcattctggcagcttccaagtcagccagctgccctaaataacccttcaacatttcttcacttttgcaaggttccacagactaagacattgggtctattccagctcattcattttatattgaaaactaatttaaaaaaaaatggtggcttcagctccagcccctttccaaaatttttcaaccccaccctgtttggatttttaattaaaatctagtagttctcttggtattaaaacacttctgtcctgtgaggttttcccaatggtgtttttcttgtaaatgtgttggacaaatgtgaagatgcgttgtagtttacccatatgcccacatttagtctctttattcctagttggtgagaaacctgtatctttctatgctgcttttatatctgtatgtattagtgatatttctctagtagttaaaaaaaaaaaaaaagaaaaaaaaaacttttttggttgtcctcaagaaaagaaaagaagaaaagaaaagaaaagaaaaagctatcttttggagctgctaactcactctcttataggattttttttcctggaaaccagcatgcttcatggaatgctagcacatggatgttttaagagggatgtacataaatgtattttgcactgtcacaatgactccctaggcatgctttctttttttcaaaccaactttttaaatatgctagagccatttcaccaattttagctgtagcatagagtagtagtggaatttttctttcttttatcctttttttatgaaaagaaattattagggacttttttaaaaaaaataaaataagatatcctactttaaaaaaaaaaaggacagtgcatgcatattgctgtaaggttgtttaagtatttctaattttacaaaaaaaaaatgaaaaaaatcattagagctccaaatgctgaggtgtagacgaacagctttaaccctgctgagtgccaggtaatacagtgttcttacaaaagggaaatcagccaaagactgatctgagggaccaaaatcggaactttgaaaagcaccagtggtactttccaaatgatcagcaggttgaacatgttcagtgtgaatccattaacccttcgttgtccagggtccagaccagaactactggttagttttgaaacacattagcccaggatgtggttgtgtgctttaagagtcggcagtgctgcaggctgtgtagacaaccaaagttgacaaggctgggaagacacttctccacatacccatacaccaggcatttctttacaaattgagtacaatccattagcttgcgggagggggaggggagcgggtaatgtagcccttggatacagttttaagtggttgtatttaagttatctgcatagaggcaatcctcctgggtttgctttatgtctcgaaaatctaagatttttcttaaaccatttcccccatattttcatataccagtcggttttgattttgttccggtggtgttgtgttctttctttcttttttttttttttttttttttcttttttcttttcaatggccttttaatttaggaagttctgatgagcaaataaatgcatctatatctatctgcatgttggaagtccatctgccagcacacctgctaaaggcaacgagaagcacataagcaggctctgaatgggttctatcttaccatggtgctcccagaatcctttgacctcattctgctttctagtctgcttgtgacactggacagtcctttcttagagctattctatgttctaggctacgaagggttaaataatcccaaaaatgaggtatgtcataccctcacgtctcgatctcaagaagctgtgatgtacgtggaacagcactgttattctgaacactagtgtgtaaagtaaggattagtacagtccgtctcattactttttaattcagggcaccctgagtgaaaaccaatacaaaaaaaaaaaaaatccctaactctgagctctatctctaattcctcttcctccttcccctcttcctcctcctcttcctctcctcctgttttgctacattctcctcagtggcaaaaagtttcactctacctctgacagcatgtatattgcaccagtagctaacaaaaactggtctagtcaaaccaaatgggcacaaaagaaccaggataccaaaagtcatacagctgcaaaccatatcacttcttggtaacaatgcagacctcataaacctaaaaaagagaaagaaaagaaaacttttgttactttccttttttgcttgtcacttatatacaggctatgtgagagtattgatttgtaggtattacacattaaaaaaaaagttatcttcattggatagaattgaatggtggtcgctgataggaatagggcgtcctctatctcttatctctgtctcttttctctttctctttctctgtcatgagactgtgtgtgacagggccacctgtcttttttatttttatttttctgaacttttttttttttaatgtgtaggtgcatgtcttggggatttaaaaatttcaaggctggtttacttatgcaaagcatgcctatgtctggaatacttagggaaagaaagcgactccatgttgtccgaattcctcaagggacagaaaaaaaaaaattggagactgttgaaatgcagatttgaagtaattttattttaattattttgggttctgcgacattattgtgaaaaattaaagttgttgtgcaatacttaattcagacacgtgccacaagttaacggtagactaacactggggggcgggggctaggcatcacgcttttgtctgcatactcttgagcttttaagtctactatgtccgaactgtggtttcttgtttatccttttttttttttttccttagttggactgtaatgtatggtctgtcaacctgtgaatctttaaagtatgattcaggtattgttgtactctttactgtgtaataaaaatgcttaaaaaaaaaaaaaaaaaaaaa

SEQ ID NO: 84 - DNA, 2971 bp, Mus musculus
agactggcatatgatgggaggcagccaatgactccgcggcgctcctccgggggccctcagtgtgcgtttgaggagaacaaaaaagagagagagcgccgagagggggaacgagcgagggagctgagtccagagaaagagccgccgggcgctgcctcgccaaacctcgctgggaccgcggggccaccaggaggcactttggtgaaggggggggggggcgacctcggcagccgcggcgcccgaagcgacccagcgcagcgtggggcgggctgcgacctctgcttcggtggattgcatttttaattaaggattcctagcagctctttgggattttttttttccggcttccactcatgtgttgacacccgcgttcaggagagacttgccccaagtgcaccgagcgcccgggacctgagacggaattgcttttcgtgcgtgcaaaatccaagcattttgagttttgtttgggacctttttcttgctttgcttttatttctatttttattttgttgcagggatatgggagttatccacaagccttagtttcggatcctgcagggaaagcccatgtagcatagcttggcttttgaaggcagagttgtgcagacacatttgggggcacgacgcaagcgctttgtgctcgtgtaccagccgcgcaacttttgaaggctcgccggcccatgcagggtgtctctagcatcgtttcgctggtggcttccctaaggctccaaagcagctggagttgagcggtcccggcccatcgtgatccatgtagcccgctggtccctcgcggactgaggctcaacacgcgcgtgttcccggcccggcccggcccggcttggcccggcgcgagctccctcatgttgcagccctgcggtgccccttcgacgacaggctgtgcgcggtctgcacggcgccccgcggcagagcttcatgtggggctgcggcccgctcagccggcgcctcgttgagggaacggacccccggtaaccggagaccgcctcccctcccaccaccccaggcgccaaagggtatcgtatgttcaggtctaaacgttcggggctggtgcggcgactttggcgaagtcgtgtggtccctgatcgggaggaaggcagcggcggcggcggtggtgtcgacgaggatgggagcctgggcagccgagctgagcctgccccgcgggcacgagagggcggaggctgcagccgctccgaagtccgctcggtagccccgcggcggccccgggacgcggtgggaccgcgaggcgccgcgatcgcgggcaggcgccggcgcacagggggcctcccgaggcccgtgtcggagtcgggggccggggctgggggctccccgctggatgtggcggagcctggaggcccaggctggctgcctgagagtgactgcgagacggtgacctgctgtctcttctccgaacgggacgcagcaggcgcgccccgggactctggcgatccccaagccagacagtccccggagccggaggagggcggcgggcctcggagtcgcgaagcccgctcgcgactgctgcttctggagcaggagctcaagacggtcacgtactcgctgctcaagaggctcaaggagcgttcgctggacacgctgttggaggctgtggagtcccgaggcggcgtaccgggcggctgcgtgctggtgccgcgcgccgacctccgcttgggcggccagcccgcgccaccgcagctgctgctcggccgcctcttccgctggccagacctgcagcacgcagtggagctgaaacccctgtgcggctgccacagctttaccgccgccgccgacgggcccacggtgtgttgcaacccctaccacttcagccggctctgcgggccagaatcaccgccgcccccctattctcggctgtctcctcctgaccagtacaagccactggatctgtccgattctacattgtcttacactgaaaccgaggccaccaactccctcatcactgctccgggtgaattctcagatgccagcatgtctccggatgccaccaagccgagccactggtgcagcgtggcgtactgggagcaccggacacgcgtgggccgcctctatgcggtgtacgaccaggctgtcagcattttctacgacctacctcagggcagcggcttctgcctgggccagctcaacctggagcagcgcagtgagtcggtgcggcgcacgcgcagcaagatcggttttggcatactgctcagcaaggagccagacggcgtgtgggcctacaaccggggcgagcaccccatcttcgtcaactccccgacgctggatgcgcccggaggccgcgccctggtcgtgcgcaaggtgccaccgggttactccatcaaggtgttcgactttgagcgctcagggctgctgcagcacgcagacgccgctcacggcccctacgacccgcacagtgtgcgcatcagcttcgccaagggctggggaccctgctactcgcgacagttcatcacctcctgcccctgttggctggagatcctactcaacaaccacagatagcaatgcggctgccactgtgccgcagcgtcccccaacctctggggggccagcgcccagagacaccaccccagggacaacctcgccctccccccagatatcatctacctagatttaatataaagttttatatattatatggaaatatatattatacttgtaattatggagtcatttttacaacgtaattatttatatatggtgcaatgtgtgtatatggagaaacaagaaagacgcactttggcttgtaattctttcaatacagatatatttttttctttctttccctctttccttttttaaagagaattatacagtagaactaggtggaaagcctaggtttggtgtatggcttttttaaaaaatattaatgcccagaccaaaaaaaaacaaaacaaaaaacaaaaaaactaataccagtcactcttgataataaagtgtttgcattata

SEQ ID NO: 85 - DNA, 4161 bp, Mus musculus
ggacctcacggcaagctaagtaactgctggtctctcagcgagagccgggaaacccagcttcggggtcctacctcgacccctctccagcggaggagtggaggtcctaaccagccgagtaggtctctctaaatatgccccaggatgacggagcggccgccgagcgaggcggcacgcagtgacccgcaactagagggacaggacgcggccgaggcccgcatggcccccccgcacctagtcctgctcaacggcgtcgccaaggagacgagccgcgcagccccggctgagccccccgtcatcgagctaggagcgcgcagcggcgcggggggcggccctgccagtgggggcggtgccgcgagggacttaaagggccgcgacgcagtagcagccgaagctcgccttcgggtgcccaccaccgagctgtgcagacctcccggacccgccccggcgcccgcgcccgcctcggctcctgcagagctgcctggagacggccgcatggtgcagctgagcccgcccgcgctggcagcccctgccggccccggccgagcgctgctctatagccttagccagccgctcgcctcactaggcagtgggttctttggggaaccggatgccttccccatgttcaccaacaacaaccgggtgaagaggaggccctccccatatgagatggagatttctgatggtcctcacaccaaagtagtgcggcgcatcttcaccaacagccgggaacgatggcggcagcagaatgtgaatggggcatttgctgagctcagaaagctgatccccacccacccaccagacaagaaactaagcaagaatgagatcctccgccttgccatgaagtacatcaatttcctggccaagttactcaatgaccaggaggaggaaggcacccagcgtgccaagcctggcaaggaccccgtggtgggagctggtgggggtggggcagggggtggcatcccccctgaagaccttctacaggacgtgctttcccccaactccagctgtggcagctctctggatggggcagccagcccggacagttacacagaggagccaacacccaagcacacttcccgcagcctccatcctgccctgctgcctgccgctgatggggctggcccccggtgatgcgtctggggctgcccagggccagcaggcaggggcctttaggcccctgggttgctgggcttcagggcaggtgggatgagaagcaggtcaatggacttatgtgaacttcccttacagtttgaactttgggaagtcccaactgaccctaggctggcatttctgtttcctgcatggaaacagaagaggcaaacagagtgaagtagtaggtactttttctgaagatggcacggtcttctccctttcccaagcccaaagatttccccaatgatgaggctcaagtgtctagttttggtctagagtttgtgagccttgttttggacaagatctggggttgtcacctgttacctgaggcatccaattggcctctgccttgcctttctcccatcccgagctggctgggtggcttttgtggcagcttctgttcaaatacttaggtacccaagagctgaccatttcttgagccccatcttcacccaggtccatgttgatccatccagcttgccagctgctgtggagagtcaagctttgaggtgccttcttcagggcctggttggaaaagatggccagtaaacagacaacctgctctcaactagctggggcagaaaacccaacagccccttttgctccagggaatcagagctctctccctcactgagacttgccttcctatcctgtggctgtgaggaccaagtcagcagccttgagcatgctttgttgggctgtgtgcaggcacagaaagggaaagtgacattagggcaagcaaagcaagagtgtttggggcactgggtttcaactccagcgcatccctgtttgtgcaggagagcaaaggatagagtgctggtgcattgcatgttggtgctggggctgtgcagcccataggcttccccaggaagcccgaaggaagcttcccattgcaagatgtctgttggctgatggtctggatgtccaaatggaggacaaactgtcctgtacatactctatttgggattctgcctgaccagttgttggttggcaactctaggccatgataccacccaagatgcagcctgatgctaaggcaaggagtccagtcagaggcaaggccctggctgagacctgggcatgcacctttgctcagcttaactttaagtactctcccctcacacaactgcaggccctacgtgtgtgcatggtaggttggcttaacatgaaacgagtgccccctctaaaacttgtacatgaaagttttgtaaagcagacccttctcctccatcttttaaagacacaacccctgcaagtcattttgtaaagtggagaatccttttgtagtatggacaaataccccattgtttctcccctgtgtcaatccaggtggtgggaccgatctatcctaaagctaggcctgcctataatgaagttcaagctcatgaaacaaagtgcgcagaagtcctgtgtatctgtcattgtattggtgtcactcagttctcactctctccagggtttgcctttgagctagagagaaaatatccaaaggccccatcatgtaacgtatgagcccatcacttttctgttggagtttcagcatgatgtttcagttcacaccatgatggagcctgagtatagacttaggcagacagagactgatcctgttcctgctcccctccccttcttattaagtactgagggcccccaggtaatgggacaatgtggtcagctactaaggcattgtgattctttgtctcctatcacattctgctatcacatgaacattaaggctggttctttcccattgcgtgggagttagttcaaggggtctgacccatgaggaaatgggtccaaagctgtgtttgcttcttagtggaatgagttgggaatgaaaactgtggctctgtaggtggattctatgagagggatcacacttttgttgggagcctaagctgcagttccctacatcagctttgcagcttcactgggataaacacgctaggcggtggacggactgtgatcataacattggaagtccattgggtccgtaaagactgctctggagcacagagggccggtgcatcgcggggggggggggggcggggattgcacacacggggttctgtgttctacacccagcacaggcacaaaagaagcttcataacccttagtactgaccgtgccgtgaggttccagccagagttcaggcctgggcagttgatgtgtttgtgtcatttggattataaaatattttccattgagttatgtagatgctaatttctcaagattaatcttaatttctccccgattccaaggcaagaaacaggagtgagaaggggtgagagatttcgaaagtttctaaaggggctgggcagggaagagccagggttttggtctttgtaagatctaaggaagaccctgaattctgttctcatactccataccccatatctttcttcctctgtgtcttccttgcccttaaagaaattgcagcattccaagaacaatatctgtacaaagggggaaatgtaagcatgagaaaacattaaaaaaaaaaaacagtgatgaacataaccacagagagaatcccacccttcaagaataattcatgtttatttgtggtggcaaataacaaaatggtacaacctttatccttttccagaaacaaaaaccaagggcacagcaactagagtgagctgacagctattttggcctttttggtgggtctagccgtacttgggatcccagtggtacatgaccctctgccgaaggcttgcctcagtctgtgtacatagcacgccatgtctgtgggcaagcccagcactttgcgtcagtgtcgtactgtatgtaatgaactgtgttggtctctgtgttttttttttctgaagaagaggagtaactactccgggtaccttgatatttgtacagcctataggccaacactgcgggcgtgtgactctttattgaaaaacaaaaacaaaaaaataccagtgtggtgatgatagtgtgtgtatatatatataaggttatatggggaagatttctaaataaaagttttacaaaggg

SEQ ID NO: 86 - DNA, 949 bp, Mus musculus
cccgcggccgggcgcccatggcgttcgcgctgctgcgcccggtcggcgcgcacgtgctgtacccggacgtgaggctgctgagcgaggacgaggagaaccgcagcgagagcgacgcgtcggaccagtcgttcggctgctgcgaagggctggaggcggcacggcgtggcccgggtcccgggagcgggcggcgggcgagcaacggcgcgggccccgtggtggtggtgcggcagcgacaggcggccaacgcgcgcgagcgagaccgcacgcagagcgtgaacacggccttcaccgccctgcgcacgctcatccccaccgagcccgtggaccgaaagctgtctaagatcgagacgctgcgtctggcgtccagctacatcgcccacctggccaacgtgctgctgctgggcgacgcggccgacgacgggcagccgtgtttccgcgcggcgggcggtggcaagagcgcggtccccgccgccgacggccgtcagccgcgctccatctgcaccttctgtctcagcaaccagcgcaaggggggcagccgtcgtgacctggggggcagctgcttgaaagtgaggggtgtagccccgctccgagggcctcggcgatgaacctggatccctggttttctccaagaaggactccagagaaagaggccatgagccagcccctagctggacagaggagaagattccagaagcccaaaccactcctgcgttgtgtaaggaccggaggacaatggcctgggcacagggctcaccctggagagccacaaggacccatctagccatccaggcctagctggccagggacaaggcagaaattttagaaaaccaaagactatttttgaaagtgtgtgtgtgtgtctgtgtgtctgtgtgaatgtagtatgtgagcaaatcggggagggggtcaaaataaaggtatttttaaataaaaaaaaaaaaaaaaaaaa

SEQ ID NO: 87 - DNA, 4354 bp, Mus musculus
aaaagctgctgccgagccctaagcacgggaagcgccgagctgtgtgtccaccgccagaccagagttcttttggttggacagatctgtgagtcttcccaggagagcacccttgggctctgctcgcccctgctccttcagggatggaggcaatggcagccagcacctccctgcctgaccctggtgactttgaccggaatgtgcctcggatctgtggagtgtgtggagaccgagccacgggcttccacttcaacgctatgacctgtgaaggctgcaagggtttcttcaggcggagcatgaagcgcaaggccctgttcacctgccccttcaatggagattgccgcatcaccaaggacaaccggcgacactgccaggcctgccggctcaaacgctgcgtggacattggcatgatgaaggagttcatcctcacagatgaggaggtgcagcgtaagcgagagatgatcatgaagaggaaggaggaagaggccttgaaggacagtctgaggcccaagctgtctgaggagcaacagcacattatcgccatcctgctcgatgcccaccacaagacctacgaccccacctatgccgacttccgggacttccggcctccaattcgtgcagacgtaagtacagggagctattctccaaggcccacactcagcttctccggagactcctcctcaaactctgatctgtacaccccctcactggacatgatggaaccggccagcttttccacgatggatctgaatgaagaaggctccgatgacccctctgtgaccctggacctgtctccgctctccatgctgccccacctggctgatcttgtcagttacagcatccaaaaggtcatcggctttgccaagatgatccctggcttcagggacctcacctctgatgaccagattgtcctgcttaagtcaagtgccattgaggtgatcatgttgcgctccaaccagtcttttaccttggatgacatgtcctgggactgtggcagccaagactacaaatatgacatcactgatgtctccagagctgggcacaccctggagctgatcgaacccctcataaagttccaggtggggctgaagaagctgaacctccatgaggaagaacatgtgctgctcatggccatctgcattgtctccccagaccgacctggggtacaggatgctaagctggttgaagccattcaggaccgcctatccaacacactgcagacctacatccgctgccgccacccgcccccgggcagccaccagctctacgccaagatgatccagaagctggctgacctgcgaagcctcaatgaggagcactccaaacagtaccgttccctctccttccagccggagaacagcatgaagctcacaccccttgtgctagaggtgttcggcaatgagatctcctgaccagggtggcctgcagtggtgcctgggtagggccgctcctctggggccctgtgcccaggccctgggctggttgcagcccagcagtgcctcctgcccctcctggagttcagctcctcctctgccgtggcccgtgtctgtctggctcatcctttctcctgcccagcctcacacctgatctccctttcctgtagactgcaggttgctcctgtcccttgagatctcagttaggagacactgctgtttatttgacaaagaaactcaagtgtgggatggggggaagagggtagagggcagaggctgaaggcagagagctctgcctagggaatgcctccgccataaggggccactgcttgtgtcaagggaggcaggcagaagagatgagtccattcctcagggacaggtacctgtacctaccccgattccaaacctacctgcctggtgagaactcctgcccctgcctacaaagggtacacaacctacccatcatccctagtgtgtcccgtctcctcctgccacctgtctgtattattctgacccaggggagtcagtcactgtggggcctccttcctctgctggtatactcatggactcattcactgccaagatgaccaaatactctaccacactaaccaaggagccctccctagccctgcagttcccacctttgaggttttgttatggtaagtccccaagtccagtacctctggtaaagtggacttcttcccccatccctagaaccagggacctagaaaccaccattggaaagtccccagggaatggaatacagagcccaagcaatgaggagcaggcaaagccagcctcccagcaggacaacaaatggaggtccatctatctttgtcaccaagctgaggtgtctgaagcctggagctactgggacccctcctctcctctatagctattagatgtgtcaggaccaagttgaccccaactttcagaacgtccctagccctgtcttcatcccttcccctgccagtgccttaccacctacccaggaaagcaggtccttgagccacccaggccttacaatttgatcaccaagagtatcccagaagttatctcagcttctccatcacccccacttggagcaacagtgtcacatctccaccccacttaccaataaaagctaccctttccacccagtggaatgggatggcagaaaaccaacagtggcccagatggacctccaagtctgcttttcaggtgtaaagccagacatgagctctcccacctcagggaaaaggtagagggggcaggttagaacttgtggtacagtgatattggtccagttcctcctgactcttcactctaccacccaaaagtcaatgtagacctacgaaacggtcaggtgatgggctagggacgtatcttcaaactccatttcttcctctgtagtagcccagtccaggactttggaaccctggtctcattcaccccaaatatacttcttgctgatcatgcgttgtgggggtgtggggggacccatgcagaggaggttctcttcataagggggactcacagaaggaccttccagtgaatgtttgacctagggaatgcagccacactggaagcaccaatagagggctaaggtcagacggagcatggccccacgagccccagattccctgaaaatcctgagatcaatcacatttaacatcagaagaacccttagtgtccacctggccctatgtttcccagaattcaagcggaaaacatctgagaatggctttcccatattgcattacggcatgcagaaggctttattttaaagtcattccatcagaggtgttctcagaattagaccccacctgtactgtgttctaacactgagggtggtggcacctgattcagagcagtgtgtgtgtgtgtgaaaatactgtatattaatgattattgttactgcacacacacacacatacaaaaaaaaaaaaaaaaccctagagaaagacctttgattttagcagcagaacacattgatctggcttactactcaacaggagggaaagtctgaaagttgataagctagttcgactcctttataatgagaacctgacatcagaggagagtgtgatttacctgggctcatataattagaagcatctccaggcctggagaaaatttgaacttatggtcccgggctgctttgtagtgagcctgtttccactgctctggtagctgagccaggctatgaggaaaccctggatatcagcataaacctatgtctgggactacgatcccttttccctgggtaataaactcagccacccatcctaaggaggcaagcacttggcctctttgagtgagacttggctttaggactctctgcatcagccttgtgtgcatcaggcactggaaagcctctgtcttcccagcccttgcctctccaggaaactccacaggcctccagagaataccagtttctcagcccaaggggtccatcactgagaagtgcccaaagggaagtcagggagctgggaggaaaagacagcaggaccaagtaggattccgaagccgaactgggagagaacagcatcatctatttttattctgaatgtacaaaggcagggccttgaaagagctcacacttccgaggcggaaatgggtaccaaaatgagagatcagcaagatttcccaagataggagaatcctcctgctgcctgcgtgggagaaagtaggggaggagtccaaaccaggccaccagagatggttgagataaatgacaaatctggtggaagtcagacatggctctaaagagcttcctaagagacttcccgagagagagacaaaagtgtttgtttgttttgtttccaggagcttgtcaaataaaccaatgaaaaagctttgccgatgataataaaagatgctcacactttcatagcgcttgctacgtgacaagtaccgctgtaaataaatgcctcatgaaaacc

SEQ ID NO: 88 - DNA, 3579 bp, Mus musculus
ggggtccatgccaagatggccgcgcccacaagcaatctccgcaggtctccgccatcgaagcccccggaccctctgaagcggtgcccggggtggccgccatatcggcctctgactccatcgaggacctgacaaagccagaggcgctggatcggaggagtgcggacaaagccaggcgtccggaggagatggctacatccgagcctgcggaaagcgatgcggaatgggagcagctggaacctgtgcagagagatgtgtacaaggatacgaagctagagaactgcagcaatccagcctccatgggaaatcaagatcccaaacaagacatagtctccgtgttggaagaagaagagccatcatcgggaaaggggaaaaaagccagcccaagtagtctgaaaaaaatagcaaggcccaagacagcaggaacaagtgcaaaactccaacaagatgatgagcatagggaggaaaagcagaagtcccaaagcaaacttactaaggaagtgacactcaggaagaaaagttccaacagcaagaaaagcagtgagtatggtttgttggagaacaaaagtctccactcaaaacacactccttccgagaaaaaactgcttaagtccagttcccgtgggaagaactcgaatcagaattcagactctctgaaaaagaaacctgacacagctaatgaccacaggaaatcactcagccattctgcatctgatgtgaacaaagatgaaattccaactagaaagaaatgcgacaagttacccaacaataagttgtctgataaaggtgacaaaaaccaaaccagcaaaaaatgtgagaaagtatgccgtcatagtgcatcccataccaaggaagacaaaattcagaccggggagaaacggaaatcacactgccgtactccatctaaacctgaaaaagccccaggttctgggaaaccttatgaatgtaaccactgtgggaaggtcctcagccataaacagggactccttgaccatcaaagaactcacactggggagaaaccatatgaatgtaatgaatgtgggatagctttcagccagaagtcccaccttgttgtacatcagagaactcacactggggaaaaaccatacgagtgtgaacagtgtggcaaagcacacggacataaacatgccctcactgaccatctaagaatccatactggagaaaagccctacaaatgtaatgaatgtggcaaaacgtttagacacagctcaaaccttatgcaacacctaagatctcacacgggtgagaagccgtatgaatgtaaggaatgtggcaaatcctttagatataattcatctcttactgaacatgtgagaacacacacaggtgaaataccatacgaatgtaacgaatgtggcaaagctttcaagtatggctcatccctgactaaacatatgcggattcatacaggggagaaaccatttgaatgtaatgaatgtgggaaaacttttagcaaaaagtcacacctagttatacatcaaagaactcatacaaaggagaaaccttataaatgtgatgagtgtgggaaagcctttggacatagctcatctcttacctaccatatgagaactcatacaggtgactgcccctttgaatgtaatcaatgtggtaaggcctttaaacagattgaaggccttacccaacaccagagagttcacacaggggagaaaccctatgagtgtgttgaatgtgggaaagcctttagtcagaagtcacacctcatcgtacaccagagaactcatacaggggagaaaccctttgaatgttatgagtgtggaaaagccttcaatgcaaaatcacaacttgttattcatcagagatcccacactggagagaaaccctatgaatgtattgaatgtggtaaagccttcaagcaaaatgcctctcttaccaaacatatgaaaattcactcagaagaacaatctgaggaagaagattaatgtaggaaacctgacaactgactggtttggtattatttaaccttaaagatgttctcaatttgatgatgttagaatatctttttttaggaaatcattcctggtgatacatgagagaatttgaatatggatctttacataagatggtaataaaatttaaccttgatcccaaacctaaacagaaaatactgtatttttacacttattataaattgtctccatatttagattaaaatacgaatcttttaaaagagtgaataaggagatatcataatcagtaaagtgcttgttaagcaagtacagagatctgaattccatccctaaggattgataaaaagctagatgtgggggcatgctcttgcaatctcacaattgggaaagttgagacaggagagtcctgagaactcctgaccaaccagacttatttaatttgcaagccataggtcacaagaaaaaagaactgttttaaaaatagggtagagggttcctgagaaacagtactcaaggttgacacatgtacacacacagacacacacagacacacagacacacacacagacacacacacagacacacacagatacacacacacacacacacagacacacacacacagacacacacacagttgtgaaaatgttaacgaattagagcaacaaacagtaaaatatgacacttcatgaactttctaggtgtgtgcttaagctccacagttagtatttaatttgtatttattcttattgcatacttaccgccttgtgtaaaatacaagtgagggtgagggtatcctctggtattgtctagctttctttgttaatgtctctgttagcttctgaattagtagaaaactttcgggagttgttttgttttgtttgagacaatatctcaccatatagtcctggctagcctggaacttgttacgtgtaccaggctggccttgaacttgttatgtagaccaggctgaccttgaactcacagagatatgtctgtctctgcctcccaagtgctagatttaaagacttcagtagaaaactttaaggggttttgtctagattttgttaagaggttcataaaaaagtggaaaacagctgaaaatgtgttgatagtgagtgatacttgttatattgttaagtatagaaaatggattgttaaaccatctatatagtatgttctcattgtggacagagtgtgtatgtgtgtccctgtactctgtgtacatatatatgtgtttatccccatatatttgtatatgaatagacttttttttaacctataagaaaattaatggtggcttccactgaacacaagagtgcagcattgaggtctgacatgtttatcatgtacatgtgtctaattcaaatatgtattatataaaaatacattcatatgtgcaaattaacacaatttaaaacaatagctgagtctatttcacagtgttatttgaaaaagtgacataagatttctatgctcctttatgaacaatgaatttaagacatatggcattgaaccttgatattgaattcacaaagaatttcctatagagcacagatttttgttaacatgagtcataactgttaacaatattttatttctcttatttgctattaaaaactaaagatgaatg

SEQ ID NO: 89 - DNA, 3276 bp, Mus musculus
ctgcggggaggatccccaggagcgggagtagggttcctcgggatagaggaagggtccagtgcgaaagggcaggaaacgggctcagggatggggctggtcgggagcgaaggacgctgacagggtctgtggcctgatcacctaggagagccactcctggtggccatgagagagaccctggaggccctgaactccttgggattctcagtgggacagccagagatggctccccagagtgagcccagggatgggttcagtaatgcccaggagaagatgtcatccagaggagagagcacactgcactcctgctcagggcatgagactcctggccagaaagaaggcatccacacagaacaagctgaagctccttgtatgggcagccaggccagtaccccacagaaggccgaacctgcaggctctgtcccaggggaggaatggatgattcggaaggtgaaggtagaggatgaggatcaggaggcagaggaggaggtggaatggccccagcatctctccttccttcccagtccttttcccactcctgacttgggtcagttggctgttacgtacaaactggagccagggactccgggagcactaggtggaatcgcgctgtccgggtgggccccgatccctgagaagccttatggctgcgaggaatgcgagcggcgtttccgggatcagctaactctgcggctccaccagaggttgcaccggggtgagggtccttgtgcctgcccggactgtggccgcagcttcacgcagcgtgctcatatgctcctgcaccaacgcagccaccgcggagagcgtcccttcccgtgctcagagtgcgacaagcgcttcagcaagaaggcccacctgacccgccacctgcgcacgcacactggcgagcgtccctacccgtgcgctgagtgcggcaaacgcttcagccagaagattcacctaggctcgcatcagaagacccacaccggagagcggcccttcccctgcaccgaatgcgagaaacgtttccgcaagaagacgcatctgatccgccaccagcgtatccacaccggcgagaggccctaccagtgcacccagtgcacgcgcagcttcacacacaagcaacacctggtgcggcaccagagggtgcacgatgctgctagccgcacccggtcctctccagacattcctgttgctccccattcccccaccgcgtctcttaccccgtcccctcctgggcccaagcctttcgcttgttcccactgcgggcagagcttcggctggaaaaagaacctcgctacgcaccagagtctgcatctcaccgagggtcgtccttttgggtgcgatgaatgtgcactcggcaccaacgtggaccccgccgccgagccctcggcctgcactccccatgcgcctgactgtggaccgggttcggggcccgcggcaccccagcgcaccacctccagcgagcgctccttcttctgcccggactgcgggcgaggctttgcccacgggcagcacctggcccgccaccggcgggtgcacacgggcgaaaggccatttgcctgtgctcagtgtggccgccgcttcggctcacgacccaatctagtcgcccactcccgggcccatagcggcgccagaccttttgcctgtgcgcaatgtggccgccgcttcagtcgcaaatctcacctgggccgccaccaggcagtgcacactggtagtcgcccccacgcctgcgccgtctgcgcccgctgcttcagctctaaaaccaacctggtccgccatcaggcaatccatacaggttcccgccccttttcctgccctcagtgcgccaagagcttcagccgcaagacccatctggtgcggcaccaacgcatccatggggacgcggccctcccagccccagcctcgaacctctctgctccagcctggtccaatccctccgaggtggtaccacctccaatctttttctaagcgaagttctgaccctcatcctgcctttcctgacagatttgggagactcatatctggagacttagtgggttttcaacattcacacctaagttgctgccaaagcaccaggacacttgggccatttctttaattgtcctttctttaatgttagttgagaagtccagaaaaacttagctcttgacagttgtgacaacacacagagactatccttgcagataccacgctgagaaacagacatttatggggatcagccaggtcaaggggagattgactgagcaagagacctctaaatagaggataagaatagttatttatgtacttgaagttcattaaagttcagatctgaggttctggagctgttaactgagccactgaaggaaaacctgactctttattcgctgccacttccatccacaggaaaaagaccttttctgtcagtccagggacgtaaacttagctaggtggtttttcccaacaaagtgcaagatgcacacaggccaaacaactaccatggtcccgcaagtgcgcgcgcgcgtgtgtgtgtgtgtgtgtgtgtgtgtgtgtgtgtgtgtgacagaaatttggctcagagggagtaggtgaggaccagaggggacaggctgctcttgggcgctcccaggggaccagcactgttactctaggtgctgctgcggctccataccacctgtttccaggccttcaagaggcttcccagaagcatccttacctatctctgcctaggaggtacagaaggaggcagtgcgtgaacaggccataaaggttgtttgtgtatcacttttcccaccagttgtctccttttgaggactgcttgagcctgcaggcactgcaactgtacaccccgctgtgactgaagcacaggggctgggctgcctgacagtgaaggagcagtcagggcatggggtgtgagtttagagagaactattttgggttcaacctcacagaggacttggagggaatcctttactctcccggaacttcaatttctcattaaaatgagagctggtgttgatgaaagctgagagtactttctatcctattgttagattactaacaacaaaaatacccaaggcaatggtctcctgggtgctaagcactgggaaggatcaggagccatcacagcttaagtaggtttcctggtttgtgttactgttgctgggataaaatactgaccttggctttccaagggtggctttcacgtctacattgatgcccctggagtgatagtcaacgttttctttgctgttcattgttgaaccaataaagtctgtgaaccctaaagaaaaaaatactgaccttgaaaaaaaaaaa

SEQ ID NO: 90 - DNA, 6152 bp, Mus musculus
cgggaccccctcctccctcccgctccccgcgctctcgccttttaatcatgcccctctgtctgtgtgtgagtgcaggcaggctgacaatgatttcctcagtgattacgtacagagcgagtccctgcgggttaggggccccctctggagccatcctgatggctttgggggccttgcttccattttccattattatgtggactaccggagcgacagcgcagtccaagaccttgcaggatgtctcgccgcaagcaagcgaaaccgagatccctcaaagaccccaactgtaaacttgaagacaagattgaagatggggaggcagtagactgcaagaagaggccagaagacggggaggagttggaggaggacgctgtgcacagctgtgacagctgcctccaggtgtttgagtcactgagcgacatcacagagcacaagatccatcagtgccagctgacagatggcgttgacgttgaggacgaccccagctgctcttggccagcctcctcgccttccagtaaggaccagacttcccccagccatggagaagggtgtgatttcggagaggaagaaggtggccctggactgccttacccatgccagttctgtgacaagtcctttagccgcctcagctacctaaagcaccatgaacagagccacagtgacaagctgcccttcaagtgcacctactgcagtcggctgttcaaacacaagcggagccgtgaccgccacataaagctccacactggggataagaagtatcactgtagcgagtgtgacgcagccttctcccggagcgatcatctcaagatccacttaaagactcacacctccaacaagccatataaatgtgccgtctgccgcagagggttcctctcctctagctccttgcacggacatatgcaggttcatgagcggaacaaggacggctcccagtctggctccaggatggaggactggaagatgaaggacactcagaagtgcagccagtgcgaggaaggctttgacttcccggaagacctccagaagcacattgcagaatgccaccccgagtgctcccctaatgaggaccgagcagccctccagtgcatgtactgccacgagctgtttgtggaggagacgtccctcatgaaccacatcgagcaggtacacgggggtgagaagaagaactcttgcagcatttgctcagagagcttcctcacggtcgaggagctgtatagccacatggacagtcaccagcagccagagtcctgcaatcacagcaacagcccttccctggtcactgtgggttacacctcagtgtccagcacgactccagactcgaacctctcagtggacagctcgaccatggtagaggctgcaccaccgattccaaagagccgcgggaggaagcgagctgctcagcagacctccgatatgactggcccctctagtaagcaggcgaaagtcacctacagctgtatttactgcaacaagcagttattttccagtctcgcggttctgcagattcacttgaaaactatgcatttagataagcctgagcaggctcatatctgtcagtattgcttggaggtcttaccctcactctataacctaaatgaacatcttaaacaagtgcacgaagctcaggaccccggcctgattgtttcggccatgcctgccattgtttaccagtgcaacttctgttccgaagttgtcaatgacctcaacacccttcaggagcacatccgatgttctcatgggtttgccaatcccgcggccaaggacagcaatgcgttcttttgtccccattgttacatggggtttctcactgactcttcacttgaagagcatataagacaggtccattgtgacctcagtggctcccggtttgggtctcctgtgcttgggactccaaaagaaccggtggttgaagtctactcctgttcctattgtacaaattcgccaatattcaacagtgttcttaaactgaataagcatattaaagagaatcataaaaacattcccttggccctgaattatattcacaatgggaagaaatcccgggccttgagccctttgtcccctgtggctatagaacaaacaactcttaagatgatgcagactgtgggaggcgggcctgcccgcgcctctggagagtatatctgtaatcagtgtggtgctaagtacacgtccctagacagctttcagactcacctcaaaacccatttggacaccgtgctgccaaaactgacctgccctcagtgtaacaaagaattccccaaccaagagtccttgctaaagcacgtgaccatccactttatgatcacctcaacctactacatctgtgagagctgtgacaagcagttcacctccgtggatgacctccagaagcacctgctggacatgcacaccttcgtcttcttccgctgcacactctgccaggaagtgttcgactccaaggtctctatccagctgcacttggccgtgaagcacagtaacgagaagaaggtgtaccgctgcacgtcctgcaactgggacttccgcaacgagaccgacctgcagctgcacgtgaagcacaaccacttggagaaccaaggcaaagtgcacaagtgcatcttctgcggcgagtcgtttggcaccgaggtggagctgcagtgccacatcaccacccacagcaagaagtacaactgcaggttctgcagcaaagccttccacgccgtcatcctgctggagaagcacctgcgggagaaacactgtgtgtttgaaaccaagacccccaactgtggcaccaacggggcctcggagcaagtgcagaaggaggaggctgagctgcagaccttgctcaccaacagccaggagtcccataacagccatgatgggagtgaggaggacgtggacagctctgaacccatgtatggctgtgacatctgcggggcggcctacaccatggagacgttgctgcagaaccaccagctccgagaccacaacatcaggcccggagaaagcgccatcgtgaagaagaaggcggagctcattaaagggaattacaagtgtaacgtttgctcgagaaccttcttctccgaaaacgggctccgtgaacacatgcagacccacttaggcccggtcaaacactacatgtgccctatctgtggagagcgcttcccttccctgttaactctcacagagcacaaagtcacacacagcaagagcctggataccggaaactgtcgtatttgtaagatgcccctgcagagcgaagaggagtttttggagcactgccagatgcaccctgacctgaggaattccctgactgggtttcgttgtgtggtgtgtatgcagactgtgacctccaccttggaactcaaaatccacgggaccttccacatgcagaagacggggaatgggtcctccgtgcagaccaccgggcgtggccagcacgttcagaaactgtataagtgcgcatcttgcctcaaagagttccgttccaagcaagatctggtgaaactcgacatcaacggcctgccatatggtctgtgtgccggctgtgtgaatctcagtaagagcagtagcccgggcctcagtctccctcctggtgccagcaggccaggcttgggccagaatgagagtctgagcgccatggaaggaaaaggcaaggcggggggactgaagacgcgctgttcaagctgcaatgtcaagtttgagtctgaaagcgaactgcagaaccacatccagacggtgcaccgggagcttgtgccagacgccaacagcacacagttgaaaacgccccaagtgtcacccatgcccagaatcagtccctcccagtccgatgagaagaagacctaccagtgtatcaagtgtcagatggttttctacaacgagtgggacatccaggttcatgtggcaaatcacatgattgatgaagggctgaaccatgagtgcaaactctgcagccagacctttgactcccccgccaaacttcagtgtcacctgatagagcacagcttcgaagggatgggtggtaccttcaagtgccccgtctgtttcacagtgtttgttcaagctaacaagttgcaacagcatatattctctgcccatggacaagaagacaagatctatgactgcacacaatgcccacagaagtttttcttccaaacagagctacagaatcatacgatgactcagcacagcagttagttcatgtatgcacggcctctcaaggagaaggacttttgtggcacaaaaagggaacatgttttactctttgcacgaaactcccattgttaatgtatactcttcagaagcattgtattgtatcatacaacgtgtactatcaaaactgttggatgttcatgtgttggaacttttgagcaccagatagacagactccttgtatataaagtgttgcacatgtattatgtcgtcgatgctaaaatggtcttataaagacaagtggacttgggccctattcaggcaagattttcaaagtgaacaaaatggcttaagagaaaatgttttcaaggaagacagaacttaaatgaactgttctcatgtgactatggttggacttccttttatgtacacttaagcctagaatttctcttttggtatatcaatggttaaaacccaagactattttttactgctggagattctcgtgaactatgaagagacattctcacagaacagaaccccacatctggatacggcccatatatatatttgtaagccttgcagtgtgacaggtagcctcaccatatatgcaatagttgttatgtagactgtcaaagatttttttttcctggctacatttgaagctttgagtgttcaaggttttccttaatgatttcacacagccaaattcttgaatcagttgaactaacctgtatgttactgctctcaatgtttaccctgcggcctgaacctggagattactggaattgttttccaagaggaaataaattcagtttaccatcagctgtgagtgtatttgtgtgttttccttttctagttagtcttaccattaagaaagaaagcaagaaaaggaaatctaatgcctgcacccttggctatgaaacttgctccagagctgattgcaggcatagcctcaatcatatttaaccttagcacaccgggccagatgcagctgggaagagatccatcatgcggcacaggaagaaagcagacccagtctatttgaactgtcttagatctaccaggtcacattgtacttgaagagtctgacatcacagatggatgtgatggctgttttctgcataatttgcagcccaattcacatcatgctgtgcagatctgaactcgtggtggatgactgcaattggagcggctgtttggcttatgtgtacttaagaagcagcgtcgttccaaagacaaagtgcagagcatcatcgagtcagagggctgttagttctgcttctgctgccgactcccagtggagccttctccaaatggcttcatctcctggcctgggatcacccgaccagggaaggtaatggaacttggtcatcagcctttcgacagggaggagatggctctatgagatgccttccgcagcctaccacaaaatgctgaaaaagagaagcccctgatttcagagctttttgtgcaattttttaaagatgagaagggtggcattgtgtgccctggaccttgatagacgtcattagtctgagtagtgtaagtatgaactccacactcacggagcgcattcatggcatcgtgcttaaaatggttaccacactctgagtcctatgacctcttttaaacttggaagatctcaagcaaatagcaataatcaccacagaactttgaggtaagaaaaatgcaacctggactgatggatgtgtgaccccttgtcaccaatggactagcctgtgacagccacatctccagcatcacaacgcagggtcactgccccatggttctaacccttaacctccttgacattcccttgcaggacttgcttgttacatcttggcgtgctggtccctgaagcctcaagcttgggctctcctaagaatttctgggtaatgctggttgcagaccacactttgagaaccactaccctacctcattcacaagtcatggaatgaaccaagtgtgaataaccaagcacatgatattatttgttgtgttttcaataaaaaaatttttttaaa

SEQ ID NO: 91 - DNA, 5512 bp, Mus musculus
ctttctccccaccccaggggtgtcttccattcttttgtggctcagtttaaggcgaaaagggctccaaaccactaactaacagaagggagccctttcttccacctcctgggagaatctcagattgaatttatctgaagatagcgtgctctcttcttacttattgccaccattacgaggaggacagcacaaccaccaccttggcttcaagatcctgggtagagaggctcacgggcatttttttttcaaccatctttggcgaggccttgcatccttccactccagcctggtgactggggctgcttttaaccctttcctatttgcagagaatgcaactgtgtgacagtaactgaacactgggccaaagtcttttcaaaaggtcaaggttcacaagaactgatcaaattcatgaccatgggggatatgaagaccccagactttgatgacctcttggcagcatttgacataccagatatggtcgatcccaaagcagcgattgagtccggacacgatgaccatgagagccacattaagcagaatgctcacgtggatgacgactctcacaccccatcatcctcagacgtcggcgtcagtgtgattgtgaagaatgtccgcaacatcgactcctccgagggggtggaaaaagatggccacaatcccacaggcaatggtttgcataatgggttcctcacggcatcctctcttgacagctatggtaaggatggagccaagtccttaaaaggagacacacctgcctcggaggtgactcttaaggacccggcattcagccagttcagccccatctccagcgccgaggagtttgaggacgatgagaagatagaggtggacgacccgcctgataaggaggaggcgcgggccggtttcagatcgaatgtgctgacgggctcagcaccccagcaggacttcgacaaactgaaggcacttggaggggaaaactccagcaagactggagtctctacatcaggccacacggataaaaacaaggtcaagagggaggcagaaagcaattctataaccctgagtgtttatgagccatttaaggtcagaaaagcagaggataagttgaaggagaactctgagaagatgcttgagagcagggtccttgacgggaagccgagctccgagaagagcgactccggcatcgctgctgccgcatcttccaaaacgaagccgtcctccaagctctcctcgtgcatagcggccattgcggcgctcagcgctaaaaaggctgcgtccgactcctgcaaagagcctgtggccaactccagggaagcctccccgttaccaaaagaagtgaatgacagtcccaaagctgccgacaagtctcccgagtcccagaatctcatcgatggcaccaagaaggcctccctgaagccatcagacagtcccaggagcgtatccagtgagaacagcagcaaagggtcaccatcctcacccgtgggctctaccccagccatccccaaagtccgcatcaagaccatcaagacatcgtctggggagatcaagaggactgtgaccagagtgctgccagaagtggacctggactctggaaagaagccttctgagcaggcagcgtccgtgatggcgtctgtgacatcactcctgtcatcttcagcatcagccacggtcctctcctccccgcccagggcacctctgcagacggccatggttacaagtgcagtttcctctgcagagctgacccccaaacaggtcaccatcaagcccgtggcgacagcttttcttcccgtgtctgccgtcaagacggcagggtctcaagtcatcaatctgaagctcgccaacaacacaacggtgaaagccacggtcatatccgccgcctctgttcagagtgccagtagcgccatcatcaaagctgccaatgccatccagcagcaaaccgttgtggtgccggcatccagcctggccaatgccaaactcgtgccaaagactgtgcaccttgccaaccttaaccttctgcctcagggtgcccaggccacctctgaactccgccaagtgctcaccaaacctcagcagcaaataaagcaggcaataatcaatgcagcggcctcgcagccacctaagaaggtgtctcgggtccaggtggtgtcgtccttgcagagttctgtggtggaagctttcaacaaggtgctgagcagcgtcaacccagtcccggtttacacccccaacctcagtcctcctgccaacgcagggatcacgttaccgatgcgtgggtacaagtgcttggagtgcggggacgcctttgccctggagaagagcctgagccagcactacgacaggcgaagcgtgcgcatcgaagtgacgtgcaaccactgtaccaagaaccttgttttttacaacaaatgcagcctcctttctcacgcccgcgggcataaggagaaaggcgtggtgatgcagtgctcccacctgatcctaaagccggtcccggcagaccagatgatagttcctccatccagcaatactgctgcttccactctgcagagctctgtgggagctgccacacacactgtcccaaaagtccagcctggcatagccggggcagttatctcagctccggcaagcacacccatgagcccagccatgcccctagacgaagacccctccaagctctgtagacacagtctcaagtgtttggagtgtaatgaagtcttccaggatgagccgtccctggccacacatttccagcacgctgcagacaccagtggacaacaaatgaagaagcacccgtgccgccagtgtgacaagtctttcagctcctcccacagcctgtgccgccacaatcgcatcaagcacaaaggcatcaggaaagtttacgcctgctcgcactgcccagactcccggcggaccttcaccaagcggctgatgctggagaggcacatacagctgatgcacgggatcaaggaccctgatgtaaaagagctgagtgatgacgctggtgatgttaccaacgatgaggaggaggaggcggagataaaggaggacgccaaggttcccagtcccaagcggaagttggaggagccggttttagagttcaggcctcccagaggagccatcactcagccactgaagaaactgaaaatcaatgtctttaaggtccacaagtgtgccgtgtgtggcttcaccaccgagaacctgctgcagttccacgaacacatcccacagcacaggtcggacggctcctcccaccagtgccgggagtgtggcctgtgctacacgtcccacggctccctggccaggcacctcttcatcgtgcacaagctgaaggagcctcagcccgtgtccaagcagaacggggctggggaagacagccagcaggagaacaagcccagccctgaggacgaggccgccgagggggcagcatcagacaggaagtgcaaagtgtgcgccaagacttttgaaacggaagctgccttaaacacacacatgcggacacatggcatggccttcatcaaatccaaaagaatgagttcagctgaaaaatagccacagaacttccaggaggacaacccctatccacataggaatggagaataagacgtttttgttaccaaaagttggcagtataacaagagttaccagtaccgtctaggctgtcgccacagactccctgtcctgccctctcacctctgcagatgtgtcccttcccataagtgttaaggcagtatttgagttttaaagagttttgtatatatttaaataacgttttatactctttgttacatgtttgtatcagtatttggtggaaaatgttttgaggtttctttgtgtgtgtgtgtgtgtgtgtgtgtgtgtgtgtgattaggattctcttttttttgtactgtttctttaaaatggagttcttagtaacagcggcagttcctgaattcgaaccaaccattttgtatgttacaaatttgaatgagttcaataataacaggctatcatgccttttttagtgttttttaatttttagaactcgccacataaattgtaagtgattgtgggtctcacaacactagcaacttttaagtgtcttagcaccacacgcagcgtgcctgctcctagcaaccgagggctccaaggacaacatcacccaggtgaggatgtggcctgagccaccccagacagcgtcagccttccaggccctacctctgtccacagtggacaaaagaggtcaggagttcagtagggtgtccttcagtccatctgggagagtgaggaagaagagattcaaaaagtggagacagtggagctggcgagttttctaggttagttcacaggctgacttacagggcactaggaggcaggctaggaaaagggcagcctgtggtcagagttgaaagtcataattagacctatagctctctattgtcaggttttgtcatagaactcaccaactattccgaagacatatatatacttcaagggtatcaagtatatctctggttcttgatagggacaaaagggcaggtttgacttaaccctttgctgtctacacagttgggttcctgttctgggtgctactgccaaatgttctggtacttaagtgtcgagacgcccagcctcaccaccgacttagcactgcagcagcctgtactctgcaactggccatagacgagccaccaggcttctagagtcgtctcagcaccctctcaaggtcacctgactggagcactgccgacgacgtgctcttggtcacatccctgtatagttctctgggaaagctataaatatatatattttggttattgttttgtgttttccgttacattttatatcttgtatttatcgcccgatatgttttgtacttttgttttcctaaaacaaagaaatccatgtgtgtgtatatagagacttgcatgctagactgtagtcaatgttcagttccttgaaaagtcttgctgctgtcgggtgtgcaccttacaccgccgcgactgtattcaacccatttcacatgtaaataaatgaggaacgtctggcccttgagcttcttcaagagagttattggtgggtctctgacatcatacagtcgggggagtgactcattgggggacaggctgcagaacactgcaggcttctttccccctctgaagaatggcttctggttcctatcggcgaccaacttgtcgccactctgccgctctctgttgacgacatgggtacaatccaagggtgtgagatattcctcggcttgaagtgtagttgctgttgcgtgtcacatcctcacccacttgtgtccactgcactttcacacgttcctgcatttgaaccttgcaataagcctgcgtggcagacacgcaggtccgtgcagccgtacagttgaggaggctgcgcccaggttcagctcttcgcccggtccctcatagctggtaagtggctttgcataatagaacccaaatattttgctctcaatcttgccctttgtggttatgtacatattgacaaatattcatttattcaacaaataaaagttatgtgcaaaaaaaaaaaaaaaaaa

SEQ ID NO: 92 - DNA, 4902 bp, Mus musculus
ggaccgtgctgcgcaggtgagtgcggacccattgcggttcgcggtgtgtcttggcggacagcacgcaggggctcccgcagccagcctcgcgggccaccaagtctccgtgtccacagcggtgacgcttgagtggcgaccattgtcagacccagaaaactcagcaggtggcggatcggggaagctgtgtcacttcacagaggggtagctgcgtgcgggggcgaagcggctcgaggtgacatcgcaggtctcttgacccgccgcggccctgcagctccagtccttccttgagagggagaccaggtgacagcagccatgatcccatcggtttggccccagaagtcggtgacctttgaggacgtggctgtgtacttcacccagacggaatgggatggtctgtctcctgggcagagagcgctgtacagggatgtcatgctggagaactataggaacctggcctctgtaggatttccacttctcaaacctgctgtgatctcacagctagaggaacgaaaggatctggaaaacctgtttcagctggccactggaacggactcccagggcctctggactgaatgtactgctgtccagtccggcaataatctgacaaaagaagtgcatgaagaagaaaagagtacattatttgaacttcagaaggacttttctcaggaaacaaacttttcaaaaacgtatattttgggccaacaacaggaaatccggtcagcaggaagtgagagggggagtgccagtgccaccgatggaagagctaaggcgagctctgtggaagggtgtttgtttagtcagactccacaattgtcccaggatcataccagctccactggagagctatattctgataccaaacttagtaagagtgaaagaatcactacagaggaaagacctctgaaacccagagaattagcagaggcttctcaaggcagccctcaagttaatcagcttccagagatctgtgatgtggagaagccctaccagtgtgcagaatgtggcaaagccttcagtgttaaagccaagttcgtctggcatcagagacttcataatggagagaaacctttcaaatgtgtggaatgtgggaagtgcttcagctacagttcccactacatcacacatcagacaatccacagtggagagaagccttatcagtgtaaggtatgcggcaaggccttcagtgttaatggaagtctcgttagacaccagaggatccacacaggagagaagccgtatcagtgcaaggactgcggggctggcttcggctgcagttctgcgtacattactcatcagagaacccacactggagagaagccttacgagtgtagtgactgtgggaaagccttcaatgttaacgcaaaactgattcagcatcaaagaattcacactggagagaagccttatgagtgcgatgtgtgcgggaagggcttccggtgcagcacccagcttaggcagcatcagagtatacacactggggaaaagccgcatcgctgcagcgagtgtgggaaaggcttcactaaaaatgcaaagctcattcaacatcagcgagtgcacacgggggagaagccctacgcctgcagtgactgcggaaagactttcagtgccaaagggaagctgatccagcaccagaggatccacacgggcgagcggccgtatgaatgcagtgagtgtgggaagtctttcaggtgtaactcccagcttcggcagcatctgagaatccatactggggagaagccatataagtgcagtgagtgtggcaaggccttcaacgtcaatgcgaagctgatgcagcaccggagaacccacaccggggagaagcccttcgaatgcaaggagtgcgggaaatgctttacttctaaaagaaacctgctggatcaccagcgagcccacaccggagaaaagccgtatcaatgtagcgagtgtgggaaagctttcagtatcaatgccaagttaaccagacatcagcaaatacataccagggagaagcctttcaagtgtatggaatgcgagaaagcgttcagctgtagctctgactatattgtgcaccagaggatccacactggagagaaaccctttcagtgtggcgagtgtggcaaagccttccacgtcaatgctcacttaattagacaccagagaagccatactggggagaaacctttccggtgcacagaatgtggcaagggcttcagccttagttctgactgtattatacatcagactgtccatacttggaagaaaccctatgtatgtaacgtatgtgggaaaactttcaggtttagcttccaactaagccagcaccaagatgttcacaatgaagacaaatcctaatgaagaaaaaaaaaactaaaaaaacaaaaaaaccccaacccaccaaccaaacaaacaaaaaaacaccccaaaaacaacaacaacaacaaacccacacagatacacaaaataataccaacaaacaagccaaaaccaaaccaaccaaccaaacctatgtttaataagaaacggattgtggctcatcaattattacttggaggaaaaccattagagggaaatgattgtggcagtcttcatttggaaacaatttttctcttttatttttaatcaggatgagcagctggaaaaatatatcaaaaataacgttttttaaaaatgtatatttgtttttcaagacagggtttccctgtgtagctttggctatcctggagctggctgtgtagaccaggctggcctccaactcagatgcacctgcctctgcctcctgagtgctgatgttaaaggtgtgcatcaccaccacccaactaataacttgatttatagcagtaaccagtcaggaaatataatgaaagaaaacatcctattcccagcagcaccacgaaaagtagattttctaggaattttgtggggctgaagaaaatgagggattctcaactgagggagaccaaaggaaacaaatacaagaaggcggagagaaatgaatgtattgctgcttcattttgacaacaagaatgcattacactttctttaaaagggggaaaagcaaacattaaaataaaaattgttcaaagaagcctttaaaaacatcagatccaaagtggagatgaacaagaaggaaaatgttggcaaatgaccactcaaaaataaacaaacctgtgtgtttggttgtaaatacttccaaataagtttgagttgtaaaacacagacatacacacacacacaaatggaaagacctttgtaaaatctgtgatataaagtgtaatatctagtgctcagattttaacaaaagataacttaaaaatcaatagtgacattaacagacagtattctagagtcacaagagttctaaagaaccccaaggagtgcaaattagtgaagacttgcatgcatgtcacattgagtatatgatatgcttaccccccaatcctctgcagccctcagtgaatgtagatgctgtatttgctatcgatgcctgccatacaagcatacactatattaacagcatgttagagttcctactttgtattgactgaactcttcaaacatagctacgccctgaaggaaggtatcctgctttgattttgatgctttgtgaattgggctccatgaagatgttggaattcttgagataccctttgaaatctcttaccccctttaagaaaaatgtaagccattagtaagagcacttgagctgcccagaagctgagaaacagctgttgtttctctgttgtcaactggtaatagttaagaaaagttttcttacttaaaacacctctagtaccaaatattcatgtttccacaccagttcaattgtttgaagacaccaactggatgtcctccaatctatatcaatttagtctctacccataatatcctgctagtgagatgggcttagtctgagaagactgtctctccccaccctccccacattcagacattaatgaggagtcctacattactgatttggctacagattgaaccctctgggttcagtaatttgctatggtaactcaccaaacataaattgtttaaagttaccatttactctatattgtgaatgtcacaggagcagccagatgaagagatggaacaagatccaaagggttcccacgtgaaggtgtgtttgtctttatagagttggagtgccttaccctattgagccttgtttagtgcttttatggagattacactgtgtgcccatgattgatgaagtccttggctgttacataagtttcccctgccagaagtcaacaggggttgctgaaagttccaactctgttagcacatagttggctcctttgacaatcagccctaatcctggagttagtgacactgagtctagcaacaagtcactttagcataaacagatggctgtaagagaccttaggagtaacaaaaggctttataatatttttctgctggaaacaagagagaccaaacatacttaagtcacagacagaaacccatttacttggttcacatccccttatgtctgtctgggcaatttggggatttgtttgtctttggttcagtagttaagtgggataaaatgaaaatgcagcactctggaagctgtcaatctaggaacacacacgaacattagacagtgcagaatattagtgtttttccataaagcagttctttccaaaatgtcttcatttaaaagtatgtaatggctagtgacatggctcagcaggtaaaggcgaagcccaacctgagtttcaacctctagagcccacatggtgggaaggacagactgacacacagttgtcctctggtctctgtgcatgcatgaaaacaatgtcattcttaaaaactaaaatacctatactgaaattaagcattttgatcatcttgaagtgtgcaacttgtctattcaaataaatctaataaaactgcagaattgctgag

SEQ ID NO: 93 - DNA, 1000 bp, Artificial Sequence
  Description of Artificial Sequence Synthetic primer
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 94 - DNA, 24 bp, Artificial Sequence
  Description of Artificial Sequence Synthetic primer
cctgtcctcgcccgagtccctgcc

SEQ ID NO: 95 - DNA, 32 bp, Artificial Sequence
  Description of Artificial Sequence Synthetic primer
ctcgtcccgagcccaccatctccacttcctcc

SEQ ID NO: 96 - DNA, 24 bp, Artificial Sequence
  Description of Artificial Sequence Synthetic primer
caacaaccgtatgcccatgacagg

SEQ ID NO: 97 - DNA, 25 bp, Artificial Sequence
  Description of Artificial Sequence Synthetic primer
ggaggtgggatggagggaatccttg

SEQ ID NO: 98 - DNA, 26 bp, Artificial Sequence
  Description of Artificial Sequence Synthetic primer
gggaaattgaagtccagccaagagtg

SEQ ID NO: 99 - DNA, 27 bp, Artificial Sequence
  Description of Artificial Sequence Synthetic primer
cccggtgaacaagcgagagtcggcgtc

SEQ ID NO: 100 - DNA, 24 bp, Artificial Sequence
  Description of Artificial Sequence Synthetic primer
gcatgggttcctcggtcaatgacg

SEQ ID NO: 101 - DNA, 24 bp, Artificial Sequence
  Description of Artificial Sequence Synthetic primer
gcgccctcggtcatggatctcagc

SEQ ID NO: 102 - DNA, 26 bp, Artificial Sequence
  Description of Artificial Sequence Synthetic primer
gaccctatttgtcagcggtctgcctc

SEQ ID NO: 103 - DNA, 24 bp, Artificial Sequence
  Description of Artificial Sequence Synthetic primer
ctccagaggcttcggtttcgtcac

SEQ ID NO: 104 - DNA, 26 bp, Artificial Sequence
  Description of Artificial Sequence Synthetic primer
gtggagaccggaaagtaccaggaagg

SEQ ID NO: 105 - DNA, 25 bp, Artificial Sequence
  Description of Artificial Sequence Synthetic primer
ggaggccgactttggatgggagcag

SEQ ID NO: 106 - DNA, 28 bp, Artificial Sequence
  Description of Artificial Sequence Synthetic primer
ggatgggttcagtaatgcccaggagaag

SEQ ID NO: 107 - DNA, 27 bp, Artificial Sequence
  Description of Artificial Sequence Synthetic primer
caggtttagatggagtacggcagtgtg

SEQ ID NO: 108 - DNA, 25 bp, Artificial Sequence
  Description of Artificial Sequence Synthetic primer
catcaccaaggacaaccggcgacac

SEQ ID NO: 109 - DNA, 27 bp, Artificial Sequence
  Description of Artificial Sequence Synthetic primer
cgaggaagaagagggagagaaactgtc

SEQ ID NO: 110 - DNA, 25 bp, Artificial Sequence
  Description of Artificial Sequence Synthetic primer
ggtgacctttgaggacgtggctgtg

SEQ ID NO: 111 - DNA, 27 bp, Artificial Sequence
  Description of Artificial Sequence Synthetic primer
caacgggccttcttcttcctcttcctc

SEQ ID NO: 112 - DNA, 24 bp, Artificial Sequence
  Description of Artificial Sequence Synthetic primer
ctgctccgtgctacccactcactg

SEQ ID NO: 113 - DNA, 24 bp, Artificial Sequence
  Description of Artificial Sequence Synthetic primer
cgattacctacccagcgaccactc

SEQ ID NO: 114 - DNA, 28 bp, Artificial Sequence
  Description of Artificial Sequence Synthetic primer
ccaacacttgagttcctttccgcctgtc

SEQ ID NO: 115 - DNA, 25 bp, Artificial Sequence
  Description of Artificial Sequence Synthetic primer
ccgaaagctgtctaagatcgagacg

SEQ ID NO: 116 - DNA, 25 bp, Artificial Sequence
  Description of Artificial Sequence Synthetic primer
ggcagcacccacatcagcagcagag

SEQ ID NO: 117 - DNA, 24 bp, Artificial Sequence
  Description of Artificial Sequence Synthetic primer
ctggatgaaagagtcgaggcaaac

SEQ ID NO: 118 - DNA, 24 bp, Artificial Sequence
  Description of Artificial Sequence Synthetic primer
gagtttggatgaagcgcaggccag

SEQ ID NO: 119 - DNA, 24 bp, Artificial Sequence
  Description of Artificial Sequence Synthetic primer
caagggtctccaaacgtccacaac

SEQ ID NO: 120 - DNA, 24 bp, Artificial Sequence
  Description of Artificial Sequence Synthetic primer
gccatcgaaaggaagagcctggac

SEQ ID NO: 121 - DNA, 24 bp, Artificial Sequence
  Description of Artificial Sequence Synthetic primer
gagcagagatgacgtagcccagtg

SEQ ID NO: 122 - DNA, 26 bp, Artificial Sequence
  Description of Artificial Sequence Synthetic primer
cgctctccttcgcgggcttaccctcc

SEQ ID NO: 123 - DNA, 27 bp, Artificial Sequence
  Description of Artificial Sequence Synthetic primer
gaactgggccatccgcgccatcgagac

SEQ ID NO: 124 - DNA, 28 bp, Artificial Sequence
  Description of Artificial Sequence Synthetic primer
gtcttcttcaaccatctcgactcgcagg

SEQ ID NO: 125 - DNA, 27 bp, Artificial Sequence
  Description of Artificial Sequence Synthetic primer
gggtttcgttgtgtggtgtgtatgcag

SEQ ID NO: 126 - DNA, 28 bp, Artificial Sequence
  Description of Artificial Sequence Synthetic primer
cggcagcgggaaggtgaacgggagctac

SEQ ID NO: 127 - DNA, 27 bp, Artificial Sequence
  Description of Artificial Sequence Synthetic primer
cggtcccggcagaccagatgatagttc

SEQ ID NO: 128 - DNA, 28 bp, Artificial Sequence
  Description of Artificial Sequence Synthetic primer
gcacgagaagcggatgtcaaaggacgag

SEQ ID NO: 129 - DNA, 27 bp, Artificial Sequence
  Description of Artificial Sequence Synthetic primer
ctgatgtgggaggtacgtgggagcaag

SEQ ID NO: 130 - DNA, 25 bp, Artificial Sequence
  Description of Artificial Sequence Synthetic primer
gagcgaggaccagtcactatttgag

SEQ ID NO: 131 - DNA, 27 bp, Artificial Sequence
  Description of Artificial Sequence Synthetic primer
cgtcgccgccgggtcaggtagcgattg

SEQ ID NO: 132 - DNA, 29 bp, Artificial Sequence
  Description of Artificial Sequence Synthetic primer
gcaaaggtgaacacaaggtcagtcagagg

SEQ ID NO: 133 - DNA, 25 bp, Artificial Sequence
  Description of Artificial Sequence Synthetic primer
catcctcttctggtccttcaccaac

SEQ ID NO: 134 - DNA, 26 bp, Artificial Sequence
  Description of Artificial Sequence Synthetic primer
caatttcatcgggaacagcaaccatg

SEQ ID NO: 135 - DNA, 24 bp, Artificial Sequence
  Description of Artificial Sequence Synthetic primer
ctgcacccgatttcttacggcttg

SEQ ID NO: 136 - DNA, 26 bp, Artificial Sequence
  Description of Artificial Sequence Synthetic primer
gttgacgctccaggatgttgtggttg

SEQ ID NO: 137 - DNA, 24 bp, Artificial Sequence
  Description of Artificial Sequence Synthetic primer
gtccttatcagggtcatcatcgtc

SEQ ID NO: 138 - DNA, 27 bp, Artificial Sequence
  Description of Artificial Sequence Synthetic primer
ccatgttgttctttctgcgcctcgccc

SEQ ID NO: 139 - DNA, 24 bp, Artificial Sequence
  Description of Artificial Sequence Synthetic primer
gaaagcggcaggaggaggaagagc

SEQ ID NO: 140 - DNA, 24 bp, Artificial Sequence
  Description of Artificial Sequence Synthetic primer
ctgccataggttgccacaaagttg

SEQ ID NO: 141 - DNA, 27 bp, Artificial Sequence
  Description of Artificial Sequence Synthetic primer
gtttgcccatactccttcccacgatac

SEQ ID NO: 142 - DNA, 29 bp, Artificial Sequence
  Description of Artificial Sequence Synthetic primer
cttctcgttggtgatatgctctggacctg

SEQ ID NO: 143 - DNA, 24 bp, Artificial Sequence
  Description of Artificial Sequence Synthetic primer
ccacccggacagcgcgattccacc

SEQ ID NO: 144 - DNA, 26 bp, Artificial Sequence
  Description of Artificial Sequence Synthetic primer
gcaaggcccaagacagcaggaacaag

SEQ ID NO: 145 - DNA, 24 bp, Artificial Sequence
  Description of Artificial Sequence Synthetic primer
cagcatggagagcggagacaggtc

SEQ ID NO: 146 - DNA, 26 bp, Artificial Sequence
  Description of Artificial Sequence Synthetic primer
ctgccgctgtctcttgcacttgtacc

SEQ ID NO: 147 - DNA, 27 bp, Artificial Sequence
  Description of Artificial Sequence Synthetic primer
gactaaacaaacacccttccacagagc

SEQ ID NO: 148 - DNA, 28 bp, Artificial Sequence
  Description of Artificial Sequence Synthetic primer
cattatttggactgtaccgctggcctgg

SEQ ID NO: 149 - DNA, 26 bp, Artificial Sequence
  Description of Artificial Sequence Synthetic primer
gaggctgagggttaaaggcagtggag

SEQ ID NO: 150 - DNA, 28 bp, Artificial Sequence
  Description of Artificial Sequence Synthetic primer
cgtcaggtagcgattgtagtgaaactcc

SEQ ID NO: 151 - DNA, 24 bp, Artificial Sequence
  Description of Artificial Sequence Synthetic primer
gcaggacagttctttctccgaatc

SEQ ID NO: 152 - DNA, 25 bp, Artificial Sequence
  Description of Artificial Sequence Synthetic primer
ctgccccccaggtcacgacggctgc

SEQ ID NO: 153 - DNA, 25 bp, Artificial Sequence
  Description of Artificial Sequence Synthetic primer
cgccgaagaaggatcgaaatagctc

SEQ ID NO: 154 - DNA, 25 bp, Artificial Sequence
  Description of Artificial Sequence Synthetic primer
ggtagttggaaggcagcgcgtaggc

SEQ ID NO: 155 - DNA, 24 bp, Artificial Sequence
  Description of Artificial Sequence Synthetic primer
gatgccgcacttcttggctaactc

SEQ ID NO: 156 - DNA, 24 bp, Artificial Sequence
  Description of Artificial Sequence Synthetic primer
gtcacatttggcaggtcatcatcg

SEQ ID NO: 157 - DNA, 27 bp, Artificial Sequence
  Description of Artificial Sequence Synthetic primer
ccactcgtagatgtcttgttcacacac

SEQ ID NO: 158 - DNA, 26 bp, Artificial Sequence
  Description of Artificial Sequence Synthetic primer
gtggttgttctcctgctgtagcctgg

SEQ ID NO: 159 - DNA, 25 bp, Artificial Sequence
  Description of Artificial Sequence Synthetic primer
gtggagcgagcatgtagccagttgg

SEQ ID NO: 160 - DNA, 28 bp, Artificial Sequence
  Description of Artificial Sequence Synthetic primer
cttcaggtttctgccttctttgccaatc

SEQ ID NO: 161 - DNA, 28 bp, Artificial Sequence
  Description of Artificial Sequence Synthetic primer
ggtatcgggtggtgtgttgcaggctggg

SEQ ID NO: 162 - DNA, 25 bp, Artificial Sequence
  Description of Artificial Sequence Synthetic primer
gaacaaacactgtgaaacagacggg

SEQ ID NO: 163 - DNA, 27 bp, Artificial Sequence
  Description of Artificial Sequence Synthetic primer
gcacagggtgaggaggaggctgaagag

SEQ ID NO: 164 - DNA, 27 bp, Artificial Sequence
  Description of Artificial Sequence Synthetic primer
ctcctcctcctcatcgttggtaacatc

SEQ ID NO: 165 - DNA, 27 bp, Artificial Sequence
  Description of Artificial Sequence Synthetic primer
cacatcatctactggactctccatctc

SEQ ID NO: 166 - DNA, 27 bp, Artificial Sequence
  Description of Artificial Sequence Synthetic primer
caggcaaagtcctcttcacagccaagg

SEQ ID NO: 167 - DNA, 24 bp, Artificial Sequence
  Description of Artificial Sequence Synthetic primer
ccatattctttcaccgcccactcc

SEQ ID NO: 168 - DNA, 26 bp, Artificial Sequence
  Description of Artificial Sequence Synthetic oligonucleotide
acacggccgtgtattactgtgcaagg

SEQ ID NO: 169 - DNA, 25 bp, Artificial Sequence
  Description of Artificial Sequence Synthetic oligonucleotide
cacggccgtgtattactgtgcaagg

SEQ ID NO: 170 - DNA, 20 bp, Artificial Sequence
  Description of Artificial Sequence Synthetic oligonucleotide
acacggccgtgtattactgt

SEQ ID NO: 171 - DNA, 26 bp, Artificial Sequence
  Description of Artificial Sequence Synthetic oligonucleotide
actcggccgtgtttccctgtgcaagg

SEQ ID NO: 172 - DNA, 27 bp, Artificial Sequence
  Description of Artificial Sequence Synthetic oligonucleotide
tacacggccgtgtattactgtgcaagg

SEQ ID NO: 173 - DNA, 13 bp, Artificial Sequence
  Description of Artificial Sequence Synthetic oligonucleotide
caaggaggcctta

SEQ ID NO: 174 - DNA, 11 bp, Artificial Sequence
  Description of Artificial Sequence Synthetic oligonucleotide
aggaggcctta

SEQ ID NO: 175 - DNA, 19 bp, Artificial Sequence
  Description of Artificial Sequence Synthetic oligonucleotide
gcaagacaggggctactat

SEQ ID NO: 176 - DNA, 14 bp, Artificial Sequence
  Description of Artificial Sequence Synthetic oligonucleotide
cctgatggttaggg

SEQ ID NO: 177 - DNA, 21 bp, Artificial Sequence
  Description of Artificial Sequence Synthetic oligonucleotide
gatagtagctggctttttctt

SEQ ID NO: 178 - DNA, 15 bp, Artificial Sequence
  Description of Artificial Sequence Synthetic oligonucleotide
cctgatggttagggn

SEQ ID NO: 179 - DNA, 15 bp, Artificial Sequence
  Description of Artificial Sequence Synthetic oligonucleotide
cctgatggttagggc

SEQ ID NO: 180 - DNA, 10 bp, Artificial Sequence
  Description of Artificial Sequence Synthetic oligonucleotide
tactacggta

SEQ ID NO: 181 - DNA, 14 bp, Artificial Sequence
  Description of Artificial Sequence Synthetic oligonucleotide
caaggcaccactct

SEQ ID NO: 182 - DNA, 18 bp, Artificial Sequence
  Description of Artificial Sequence Synthetic oligonucleotide
tctgcacattccaattct

SEQ ID NO: 183 - DNA, 14 bp, Artificial Sequence
  Description of Artificial Sequence Synthetic oligonucleotide
caaggcaccactct

SEQ ID NO: 184 - DNA, 16 bp, Artificial Sequence
  Description of Artificial Sequence Synthetic oligonucleotide
cnngnccccctttcac

SEQ ID NO: 185 - DNA, 18 bp, Artificial Sequence
  Description of Artificial Sequence Synthetic oligonucleotide
gtgactactttgactact

SEQ ID NO: 186 - DNA, 17 bp, Artificial Sequence
  Description of Artificial Sequence Synthetic oligonucleotide
ctttgactactggggcc

(19) United States
(12) Patent Application Publication
(10) Pub. No.: US 2016/0032317 A1
(43) Pub. Date: Feb. 4, 2016

(54) COMPOSITIONS AND METHODS FOR REPROGRAMMING HEMATOPOIETIC STEM CELL LINEAGES

(71) Applicant: CHILDREN'S MEDICAL CENTER CORPORATION, Boston, MA (US)

(72) Inventors: Derrick Rossi, Roslindale, MA (US); Jonah Riddell, Brighton, MA (US); Roi Gazit, Rishon LeZion (IL)

(73) Assignee: CHILDREN'S MEDICAL CENTER CORPORATION, Boston, MA (US)

(21) Appl. No.: 14/774,785

(22) PCT Filed: Mar. 14, 2014
(86) PCT No.: PCT/US14/29144

§ 371 Date: Sep. 11, 2015

Related U.S. Application Data
(60) Provisional application No. 61/782,037, filed on Mar. 14, 2013.

(51) Int. Cl. C12N 15/86; C12N 5/0789

(52) U.S. Cl. CPC C12N 15/86; C12N 5/0647; C12N 2730/00041; C12N 2501/60; C12N 2510/00; C12N 2506/1307; C12N 2506/11

(57) ABSTRACT

Provided herein are compositions, methods, and kits for hematopoietic stem cell induction or for reprogramming cells to the multipotent state of hematopoietic stem cells. In some embodiments, the compositions comprise at least one HSC inducing factor. Such compositions, methods and kits can be used for inducing hematopoietic stem cells in vitro, ex vivo, or in vivo, as described herein, and these induced hematopoietic stem cells can be used in regenerative medicine applications and therapies.

### CROSS-REFERENCE TO RELATED APPLICATION

This application claims benefit under 35 U.S.C. §119(e) of U.S. Provisional Application No. 61/782,037 filed Mar. 14, 2013, the content of which is incorporated herein by reference in its entirety.

### SEQUENCE LISTING

The instant application contains a Sequence Listing which has been submitted electronically in ASCII format and is hereby incorporated by reference in its entirety. Said ASCII copy, created on Mar. 14, 2014, is named 701039-076171-PCT1_SL.txt and is 506,202 bytes in size.

### FIELD OF THE INVENTION

The present invention relates to compositions, methods, and kits for reprogramming hematopoietic lineages and inducing hematopoietic stem cells.

### BACKGROUND

Hematopoietic stem cells (HSCs) are a subset of multipotent stem cells that are responsible for the ability to sustain lifelong hematopoiesis, and continuously generate myriad and various blood cell types, while maintaining adequate number of stem cells in the bone marrow. Hematopoietic stem cells give rise to all the blood or immune cell types, including monocytes and macrophages, neutrophils, basophils, eosinophils, erythrocytes, megakaryocytes/platelets, dendritic cells, T-cells, B-cells, NKT-cells, and NK-cells. Hematopoietic tissues contain cells with long-term and short-term regeneration capacities, and committed multipotent, oligopotent, and unipotent progenitors.

Transplantation of hematopoietic stem cells (HSCT) has become the standard of care for many patients with defined congenital or acquired disorders of the hematopoietic system or with chemo- radio- or, immuno-sensitive malignancies. Over the last two decades, HSCT has seen rapid expansion and a constant evolution in technology use. (Gratwohl A, et al., (2010). Hematopoietic stem cell transplantation A Global Perspective. JAMA. 303(16):1617-24).

### SUMMARY

The inventors have identified key transcription factors that can surprisingly reprogram committed cells and blood cells back into hematopoietic stem cells.

Hematopoietic stem cells (HSCs) are the best-characterized tissue-specific stem cells, yet the experimental study of HSCs remains challenging, due to the fact that they are exceedingly rare and methods to purify them are cumbersome, and vary between different laboratories. Moreover, genetic tools for specifically addressing issues related to HSC biology are lacking. In spite of wide clinical use, HSC transplantation remains a high-risk procedure, with the number of stem cells available for transplantation being the strongest predictor of transplantation success. One of the central clinical challenges of HSC transplantation arises from the fact that HSCs are exceedingly rare cells, occurring at a frequency of only 1/20,000 bone marrow cells and obtaining enough cells for transplant is challenging. Thus, an ability to expand HSC numbers prior to transplantation could overcome the problem of limited HSC numbers. Efforts to expand HSCs prior to transplant by ex vivo culturing have proven challenging and such efforts have not yet translated to the clinic. Thus, there remains a clinical need to find alternative strategies for either expanding the numbers of existing HSCs, or generating HSCs de novo from more abundant cell types.

The embodiments of the invention provide multiple applications, including kits for research use and methods for generation of cells useful for conducting small molecule screens for blood diseases. In addition, the invention provides commercially and medically useful methods to produce autologous hematopoietic stem cells and give them back to a patient in need, with or without genome editing. Transplant of hematopoietic stem cells is a critically important procedure that is currently limited for a variety of reasons.

Provided herein are compositions, methods, and kits for hematopoietic stem cell induction or for reprogramming cells to the multipotent state of hematopoietic stem cells, based, in part, on the discoveries described herein of novel combinations of transcription factors that permit dedifferentiation and reprogramming of more differentiated cells to the hematopoietic stem cell state. Such compositions, nucleic acid constructs, methods and kits can be used for inducing hematopoietic stem cells in vitro, ex vivo, or in vivo, as described herein, and these induced hematopoietic stem cells can be used in regenerative medicine applications and therapies.

For example, the methods described herein can be used to produce HSC cells for treat diseases including leukemia, lymphomas, solid tumors, aplastic anemia, congenital bone marrow failure syndromes, immune deficiencies, sickle cell disease, thalassemia and metabolic/storage diseases, such as amyloidosis.

Accordingly, provided herein, in some aspects are hematopoietic stem cell (HSC) inducing composition comprising one or more expression vectors encoding at least one, two, three, four, five, six, seven, eight, or more HSC inducing factors selected from: CDKN1C, DNMT3B, EGR1, ETV6, EVI1, GATA2, GFI1B, GLIS2, HLF, HMGA2, HOXA5, HOXA9, HOXB3, HOXB4, HOXB5, IGF2BP2, IKZF2, KLF12, KLF4, KLF9, LMO2, MEIS1, MSI2, MYCN, NAP1L3, NDN, NFIX, NKX2-3, NR3C2, PBX1, PRDM16, PRDM5, RARB, RBBP6, RBPMS, RUNX1, RUNX1T1, SMAD6, TAL1, TCF15, VDR, ZFP37, ZFP467, ZFP521, ZFP532, and ZFP612.

In some embodiments of these aspects and all such aspects described herein, the at least one, two, three, four, or more HSC inducing factors are HLF, RUNX1T1, PBX1, LMO2, PRDM5, ZFP37, MYCN, MSI2, NKX2-3, MEIS1, and RBPMS.

In some embodiments of these aspects and all such aspects described herein, the at least one, two, three, four, or more HSC inducing factors are HLF, RUNX1T1, ZFP37, PBX1, LMO2, and PRDM5.

Also provided herein, in some aspects, are hematopoietic stem cell (HSC) inducing compositions comprising one or more expression vectors comprising:

a. a nucleic acid sequence encoding HLF;

b. a nucleic acid sequence encoding RUNX1T1;

c. a nucleic acid sequence encoding ZFP37;

d. a nucleic acid sequence encoding PBX1;

e. a nucleic acid sequence encoding LMO2; and

f. a nucleic acid sequence encoding PRDM5.

In some embodiments of these aspects and all such aspects described herein, the composition further comprises one or more expression vectors comprising:

a. a nucleic acid sequence encoding PRDM16;

b. a nucleic acid sequence encoding ZFP467; and

c. a nucleic acid sequence encoding VDR.

Provided herein, in some aspects, are hematopoietic stem cell (HSC) inducing compositions comprising one or more expression vectors comprising:

a. a nucleic acid sequence encoding HLF;

b. a nucleic acid sequence encoding RUNX1T1;

c. a nucleic acid sequence encoding PBX1;

d. a nucleic acid sequence encoding LMO2;

e. a nucleic acid sequence encoding PRDM5

f. a nucleic acid sequence encoding ZFP37;

g. a nucleic acid sequence encoding MYCN;

h. a nucleic acid sequence encoding MSI2;

i. a nucleic acid sequence encoding NKX2-3;

j. a nucleic acid sequence encoding MEIS1; and

k. a nucleic acid sequence encoding RBPMS.

Provided herein, in some aspects, are hematopoietic stem cell (HSC) inducing compositions comprising one or more expression vectors comprising:

a. a nucleic acid sequence encoding ZFP467;

b. a nucleic acid sequence encoding PBX1;

c. a nucleic acid sequence encoding HOXB4; and

d. a nucleic acid sequence encoding MSI2.

In some embodiments of these aspects and all such aspects described herein, the composition further comprises one or more expression vectors comprising:

a. a nucleic acid sequence encoding HLF;

b. a nucleic acid sequence encoding LMO2;

c. a nucleic acid sequence encoding PRDM16; and

d. a nucleic acid sequence encoding ZFP37.

Provided herein, in some aspects, are hematopoietic stem cell (HSC) inducing compositions comprising one or more expression vectors comprising:

a. a nucleic acid sequence encoding MYCN;

b. a nucleic acid sequence encoding MSI2;

c. a nucleic acid sequence encoding NKX2-3; and

d. a nucleic acid sequence encoding RUNX1T1.

In some embodiments of these aspects and all such aspects described herein, the composition further comprises one or more expression vectors comprising:

a. a nucleic acid sequence encoding HOXB5;

b. a nucleic acid sequence encoding HLF;

c. a nucleic acid sequence encoding ZFP467;

d. a nucleic acid sequence encoding HOXB3;

e. a nucleic acid sequence encoding LMO2;

f. a nucleic acid sequence encoding PBX1;

g. a nucleic acid sequence encoding ZFP37; and

h. a nucleic acid sequence encoding ZFP521.

Provided herein, in some aspects, are hematopoietic stem cell (HSC) inducing compositions comprising one or more expression vectors comprising:

a. a nucleic acid sequence encoding HOXB4;

b. a nucleic acid sequence encoding PBX1;

c. a nucleic acid sequence encoding LMO2;

d. a nucleic acid sequence encoding ZFP467; and

e. a nucleic acid sequence encoding ZFP521.

In some embodiments of these aspects and all such aspects described herein, the composition further comprises one or more expression vectors comprising:

a. a nucleic acid sequence encoding KLF12;

b. a nucleic acid sequence encoding HLF; and

c. a nucleic acid sequence encoding EGR1.

Provided herein, in some aspects, are hematopoietic stem cell (HSC) inducing compositions comprising one or more expression vectors comprising:

a. a nucleic acid sequence encoding MEIS1;

b. a nucleic acid sequence encoding RBPMS;

c. a nucleic acid sequence encoding ZFP37;

d. a nucleic acid sequence encoding RUNX1T1; and

e. a nucleic acid sequence encoding LMO2.

In some embodiments of these aspects and all such aspects described herein, the composition further comprises one or more expression vectors comprising:

a. a sequence encoding KLF12; and

b. a sequence encoding HLF;

Provided herein, in some aspects, are hematopoietic stem cell (HSC) inducing compositions comprising one or more expression vectors comprising:

a. a nucleic acid sequence encoding ZFP37;

b. a nucleic acid sequence encoding HOXB4;

c. a nucleic acid sequence encoding LMO2; and

d. a nucleic acid sequence encoding HLF.

In some embodiments of these aspects and all such aspects described herein, the composition further comprises one or more expression vectors comprising:

a. a nucleic acid sequence encoding MYCN;

b. a nucleic acid sequence encoding ZFP467;

c. a nucleic acid sequence encoding NKX2-3

d. a nucleic acid sequence encoding PBX1; and

e. a nucleic acid sequence encoding KLF4.

In some embodiments of these aspects and all such aspects described herein, the one or more expression vectors are retroviral vectors.

In some embodiments of these aspects and all such aspects described herein, the one or more expression vectors are lentiviral vectors. In some embodiments, the lentiviral vectors are inducible lentiviral vectors.

Also provided herein, in some aspects, are hematopoietic stem cell (HSC) inducing compositions comprising modified mRNA sequences encoding at least one, two, three, four, five, six, seven, eight, or more HSC inducing factors selected from: CDKN1C, DNMT3B, EGR1, ETV6, EVI1, GATA2, GFI1B, GLIS2, HLF, HMGA2, HOXA5, HOXA9, HOXB3, HOXB4, HOXB5, IGF2BP2, IKZF2, KLF12, KLF4, KLF9, LMO2, MEIS1, MSI2, MYCN, NAP1L3, NDN, NFIX, NKX2-3, NR3C2, PBX1, PRDM16, PRDM5, RARB, RBBP6, RBPMS, RUNX1, RUNX1T1, SMAD6, TAL1, TCF15, VDR, ZFP37, ZFP467, ZFP521, ZFP532, and ZFP612, wherein each cytosine of each said modified mRNA sequence is a modified cytosine, each uracil of each said modified mRNA sequence is a modified uracil, or a combination thereof.

In some embodiments of these aspects and all such aspects described herein, the at least one, two, three, four, or more HSC inducing factors are HLF, RUNX1T1, PBX1, LMO2, PRDM5, ZFP37, MYCN, MSI2, NKX2-3, MEIS1, and RBPMS.

In some embodiments of these aspects and all such aspects described herein, the at least one, two, three, four, or more HSC inducing factors are HLF, RUNX1T1, ZFP37, PBX1, LMO2, and PRDM5.

Provided herein, in some aspects, are hematopoietic stem cell (HSC) inducing compositions comprising

a. a modified mRNA sequence encoding HLF;

b. a modified mRNA sequence encoding RUNX1T1;

c. a modified mRNA sequence encoding ZFP37;

d. a modified mRNA sequence encoding PBX1;

e. a modified mRNA sequence encoding LMO2; and

f. a modified mRNA sequence encoding PRDM5;
wherein each cytosine of each said modified mRNA sequence is a modified cytosine, each uracil of each said modified mRNA sequence is a modified uracil, or a combination thereof.

In some embodiments of these aspects and all such aspects described herein, the composition further comprises one or more of:

a. a modified mRNA sequence encoding PRDM16;

b. a modified mRNA sequence encoding ZFP467; and

c. a modified mRNA sequence encoding VDR;
wherein each cytosine of each said modified mRNA sequence is a modified cytosine, each uracil of each said modified mRNA sequence is a modified uracil, or a combination thereof.

Provided herein, in some aspects, are hematopoietic stem cell (HSC) inducing compositions comprising

a. a modified mRNA sequence encoding HLF;

b. a modified mRNA sequence encoding RUNX1T1;

c. a modified mRNA sequence encoding PBX1;

d. a modified mRNA sequence encoding LMO2;

e. a modified mRNA sequence encoding PRDM5

f. a modified mRNA sequence encoding ZFP37;

g. a modified mRNA sequence encoding MYCN;

h. a modified mRNA sequence encoding MSI2;

i. a modified mRNA sequence encoding NKX2-3;

j. a modified mRNA sequence encoding MEIS1; and

k. a modified mRNA sequence encoding RBPMS;
wherein each cytosine of each said modified mRNA sequence is a modified cytosine, each uracil of each said modified mRNA sequence is a modified uracil, or a combination thereof.

Provided herein, in some aspects, are hematopoietic stem cell (HSC) inducing compositions comprising

a. a modified mRNA sequence encoding ZFP467;

b. a modified mRNA sequence encoding PBX1;

c. a modified mRNA sequence encoding HOXB4; and

d. a modified mRNA sequence encoding MSI2;
wherein each cytosine of each said modified mRNA sequence is a modified cytosine, each uracil of each said modified mRNA sequence is a modified uracil, or a combination thereof.

In some embodiments of these aspects and all such aspects described herein, the composition further comprises one or more of:

a. a modified mRNA sequence encoding HLF;

b. a modified mRNA sequence encoding LMO2;

c. a modified mRNA sequence encoding PRDM16; and

d. a modified mRNA sequence encoding ZFP37.
wherein each cytosine of each said modified mRNA sequence is a modified cytosine, each uracil of each said modified mRNA sequence is a modified uracil, or a combination thereof.

Provided herein, in some aspects, are hematopoietic stem cell (HSC) inducing compositions comprising

a. a modified mRNA sequence encoding MYCN;

b. a modified mRNA sequence encoding MSI2;

c. a modified mRNA sequence encoding NKX2-3; and

d. a modified mRNA sequence encoding RUNX1T1;
wherein each cytosine of each said modified mRNA sequence is a modified cytosine, each uracil of each said modified mRNA sequence is a modified uracil, or a combination thereof.

In some embodiments of these aspects and all such aspects described herein, the composition further comprises one or more of:

a. a modified mRNA sequence encoding HOXB5;

b. a modified mRNA sequence encoding HLF;

c. a modified mRNA sequence encoding ZFP467;

d. a modified mRNA sequence encoding HOXB3;

e. a modified mRNA sequence encoding LMO2;

f. a modified mRNA sequence encoding PBX1;

g. a modified mRNA sequence encoding ZFP37; and

h. a modified mRNA sequence encoding ZFP521;
wherein each cytosine of each said modified mRNA sequence is a modified cytosine, each uracil of each said modified mRNA sequence is a modified uracil, or a combination thereof.

Provided herein, in some aspects, are hematopoietic stem cell (HSC) inducing compositions comprising

a. a modified mRNA sequence encoding HOXB4;

b. a modified mRNA sequence encoding PBX1;

c. a modified mRNA sequence encoding LMO2;

d. a modified mRNA sequence encoding ZFP467; and

e. a modified mRNA sequence encoding ZFP521;
wherein each cytosine of each said modified mRNA sequence is a modified cytosine, each uracil of each said modified mRNA sequence is a modified uracil, or a combination thereof.

In some embodiments of these aspects and all such aspects described herein, the composition further comprises one or more of:

a. a modified mRNA sequence encoding KLF12;

b. a modified mRNA sequence encoding HLF; and

c. a modified mRNA sequence encoding EGR;
wherein each cytosine of each said modified mRNA sequence is a modified cytosine, each uracil of each said modified mRNA sequence is a modified uracil, or a combination thereof.

Provided herein, in some aspects, are hematopoietic stem cell (HSC) inducing compositions comprising

a. a modified mRNA sequence encoding MEIS1;

b. a modified mRNA sequence encoding RBPMS;

c. a modified mRNA sequence encoding ZFP37;

d. a modified mRNA sequence encoding RUNX1T1; and

e. a modified mRNA sequence encoding LMO2.
wherein each cytosine of each said modified mRNA sequence is a modified cytosine, each uracil of each said modified mRNA sequence is a modified uracil, or a combination thereof.

In some embodiments of these aspects and all such aspects described herein, the composition further comprises one or more of:

a. a modified mRNA sequence encoding KLF12; and

b. a modified mRNA sequence encoding HLF;
wherein each cytosine of each said modified mRNA sequence is a modified cytosine, each uracil of each said modified mRNA sequence is a modified uracil, or a combination thereof.

Provided herein, in some aspects, are hematopoietic stem cell (HSC) inducing compositions comprising

a. a modified mRNA sequence encoding ZFP37;

b. a modified mRNA sequence encoding HOXB4;

c. a modified mRNA sequence encoding LMO2; and

d. a modified mRNA sequence encoding HLF;
wherein each cytosine of each said modified mRNA sequence is a modified cytosine, each uracil of each said modified mRNA sequence is a modified uracil, or a combination thereof.

In some embodiments of these aspects and all such aspects described herein, the composition further comprises one or more of:

a. a modified mRNA encoding MYCN;

b. a modified mRNA encoding ZFP467;

c. a modified mRNA encoding NKX2-3

d. a modified mRNA encoding PBX1; and

e. a modified mRNA encoding KLF4;
wherein each cytosine of each said modified mRNA sequence is a modified cytosine, each uracil of each said modified mRNA sequence is a modified uracil, or a combination thereof.

In some embodiments of these aspects and all such aspects described herein, the modified cytosine is 5-methylcytosine and the modified uracil is pseudouracil.

In some embodiments of these aspects and all such aspects described herein, the modified mRNA sequences comprise one or more nucleoside modifications selected from the group consisting of pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, 4-methoxy-2-thio-pseudouridine, 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, 2-aminopurine, 2,6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl)adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine, inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine, and combinations thereof.

Also provided herein in some aspects, are methods for preparing an induced hematopoietic stem cell (iHSC) from a somatic cell comprising:
a. transducing the somatic cell with one or more vectors comprising a nucleic acid sequence encoding HLF, a nucleic acid sequence encoding RUNX1T1; a nucleic acid sequence encoding ZFP37; a nucleic acid sequence encoding PBX1; a nucleic acid sequence encoding LMO2; and a nucleic acid sequence encoding PRDM5, wherein each said nucleic acid sequence is operably linked to a promoter; and
b. culturing the transduced somatic cell in a cell media that supports growth of hematopoietic stem cells, thereby preparing an iHSC.

In some embodiments of these aspects and all such aspects described herein, the transducing of step (a) further comprises one or more vectors comprising one or more of: a nucleic acid sequence encoding PRDM16 a nucleic acid sequence encoding ZFP467; and a nucleic acid sequence encoding VDR.

Provided herein in some aspects, are methods for preparing an induced hematopoietic stem cell (iHSC) from a somatic cell comprising:
a. transducing the somatic cell with one or more vectors comprising a nucleic acid sequence encoding HLF, a nucleic acid sequence encoding RUNX1T1; a nucleic acid sequence encoding PBX1; a nucleic acid sequence encoding LMO2; a nucleic acid sequence encoding PRDM5; a nucleic acid sequence encoding ZFP37; a nucleic acid sequence encoding MYCN; a nucleic acid sequence encoding MSI2; a nucleic acid sequence encoding NKX2-3; a nucleic acid sequence encoding MEIS1; and a nucleic acid sequence encoding RBPMS; wherein each said nucleic acid sequence is operably linked to a promoter; and
b. culturing the transduced somatic cell in a cell media that supports growth of hematopoietic stem cells, thereby preparing an iHSC.

Provided herein in some aspects, are methods for preparing an induced hematopoietic stem cell (iHSC) from a somatic cell comprising:
a. transducing the somatic cell with one or more vectors comprising a nucleic acid sequence encoding ZFP467, a nucleic acid sequence encoding PBX1; a nucleic acid sequence encoding HOXB4; and a nucleic acid sequence encoding MSI2; wherein each said nucleic acid sequence is operably linked to a promoter; and
b. culturing the transduced somatic cell in a cell media that supports growth of hematopoietic stem cells, thereby preparing an iHSC.

In some embodiments of these aspects and all such aspects described herein, the transducing of step (a) further comprises one or more vectors comprising one or more of: a nucleic acid sequence encoding HLF, a nucleic acid sequence encoding LMO2; a nucleic acid sequence encoding PRDM16; and a nucleic acid sequence encoding ZFP37.

Provided herein in some aspects, are methods for preparing an induced hematopoietic stem cell (iHSC) from a somatic cell comprising:
a. transducing the somatic cell with one or more vectors comprising a nucleic acid sequence encoding MYCN; a nucleic acid sequence encoding MSI2, a nucleic acid sequence encoding NKX2-3; and a nucleic acid sequence encoding RUNX1T1; wherein each said nucleic acid sequence is operably linked to a promoter; and
b. culturing the transduced somatic cell in a cell media that supports growth of hematopoietic stem cells, thereby preparing an iHSC.

In some embodiments of these aspects and all such aspects described herein, the transducing of step (a) further comprises one or more vectors comprising one or more of: a nucleic acid sequence encoding HOXB5; a nucleic acid sequence encoding HLF, a nucleic acid sequence encoding ZFP467; a nucleic acid sequence encoding HOXB3; a nucleic acid sequence encoding LMO2; a nucleic acid sequence encoding PBX1; a nucleic acid sequence encoding ZFP37; and a nucleic acid sequence encoding ZFP521.

Provided herein in some aspects, are methods for preparing an induced hematopoietic stem cell (iHSC) from a somatic cell comprising:
a. transducing the somatic cell with one or more vectors comprising a nucleic acid sequence encoding HOXB4; a nucleic acid sequence encoding PBX1, a nucleic acid sequence encoding LMO2; a nucleic acid sequence encoding ZFP467; and a nucleic acid sequence encoding ZFP521; wherein each said nucleic acid sequence is operably linked to a promoter; and
b. culturing the transduced somatic cell in a cell media that supports growth of hematopoietic stem cells, thereby preparing an iHSC.

In some embodiments of these aspects and all such aspects described herein, the transducing of step (a) further comprises one or more vectors comprising one or more of: a nucleic acid sequence encoding KLF12; a nucleic acid sequence encoding HLF; and a nucleic acid sequence encoding EGR1.

Provided herein, in some aspects, are methods for preparing an induced hematopoietic stem cell (iHSC) from a somatic cell comprising:
a. transducing the somatic cell with one or more vectors comprising a nucleic acid sequence encoding MEIS1; a nucleic acid sequence encoding RBPMS; a nucleic acid sequence encoding ZFP37; a nucleic acid sequence encoding RUNX1T1; and a nucleic acid sequence encoding LMO2; wherein each said nucleic acid sequence is operably linked to a promoter; and
b. culturing the transduced somatic cell in a cell media that supports growth of hematopoietic stem cells, thereby preparing an iHSC.

In some embodiments of these aspects and all such aspects described herein, the transducing of step (a) further comprises one or more vectors comprising one or more of: a nucleic acid sequence encoding KLF12; and a nucleic acid sequence encoding HLF.

Provided herein, in some aspects, are methods for preparing an induced hematopoietic stem cell (iHSC) from a somatic cell comprising:
a. transducing the somatic cell with one or more vectors comprising a nucleic acid sequence encoding ZFP37; a nucleic acid sequence encoding HOXB4; a nucleic acid sequence encoding LMO2; and a nucleic acid sequence encoding HLF; wherein each said nucleic acid sequence is operably linked to a promoter; and
b. culturing the transduced somatic cell in a cell media that supports growth of hematopoietic stem cells, thereby preparing an iHSC.

In some embodiments of these aspects and all such aspects described herein, the transducing of step (a) further comprises one or more vectors comprising one or more of: a nucleic acid sequence encoding KLF12; and a nucleic acid sequence encoding HLF.

Provided herein, in some aspects, are methods for preparing an induced hematopoietic stem cell (iHSC) from a somatic cell comprising:
a. transducing the somatic cell with one or more vectors comprising a nucleic acid sequence encoding ZFP37; a nucleic acid sequence encoding HOXB4; a nucleic acid sequence encoding LMO2; and a nucleic acid sequence encoding HLF; wherein each said nucleic acid sequence is operably linked to a promoter; and
b. culturing the transduced somatic cell in a cell media that supports growth of hematopoietic stem cells, thereby preparing an iHSC.

In some embodiments of these aspects and all such aspects described herein, the transducing of step (a) further comprises one or more vectors comprising one or more of: a nucleic acid sequence encoding MYCN; a nucleic acid sequence encoding ZFP467; a nucleic acid sequence encoding NKX2-3; a nucleic acid sequence encoding PBX1; and a nucleic acid sequence encoding KLF4.

In some embodiments of these aspects and all such aspects described herein, the somatic cell is a fibroblast cell.

In some embodiments of these aspects and all such aspects described herein, the somatic cell is a hematopoietic lineage cell.

In some embodiments of these aspects and all such aspects described herein, the hematopoietic lineage cell is selected from promyelocytes, neutrophils, eosinophils, basophils, reticulocytes, erythrocytes, mast cells, osteoclasts, megakaryoblasts, platelet producing megakaryocytes, platelets, monocytes, macrophages, dendritic cells, lymphocytes, NK cells, NKT cells, innate lymphocytes, multipotent hematopoietic progenitor cells, oligopotent hematopoietic progenitor cells, and lineage restricted hematopoietic progenitors.

In some embodiments of these aspects and all such aspects described herein, the hematopoietic lineage cell is selected from a multi-potent progenitor cell (MPP), common myeloid progenitor cell (CMP), granulocyte-monocyte progenitor cells (GMP), common lymphoid progenitor cell (CLP), and pre-megakaryocyte-erythrocyte progenitor cell.

In some embodiments of these aspects and all such aspects described herein, the hematopoietic lineage cell is selected from a megakaryocyte-erythrocyte progenitor cell (MEP), a ProB cell, a PreB cell, a PreProB cell, a ProT cell, a double-negative T cell, a pro-NK cell, a pro-dendritic cell (pro-DC), pre-granulocyte/macrophage cell, a granulocyte/macrophage progenitor (GMP) cell, and a pro-mast cell (ProMC).

Also provided herein, in some aspects, are methods of promoting transdifferentiation of a ProPreB cell to the myeloid lineage comprising:
a. transducing a ProPreB cell with one or more vectors comprising a nucleic acid sequence encoding ZFP467, a nucleic acid sequence encoding PBX1; a nucleic acid sequence encoding HOXB4; and a nucleic acid sequence encoding MSI2; wherein each said nucleic acid sequence is operably linked to a promoter; and
b. culturing the transduced ProPreB cell in a cell media that supports growth of myeloid lineage cells, thereby transdifferentiating the ProPreB cell to the myeloid lineage.

In some embodiments of these aspects and all such aspects described herein, the transducing of step (a) further comprises one or more vectors comprising one or more of: a nucleic acid sequence encoding HLF, a nucleic acid sequence encoding LMO2; a nucleic acid sequence encoding PRDM16; and a nucleic acid sequence encoding ZFP37.

Also provided herein, in some aspects, are methods of increasing survival and/or proliferation of ProPreB cells, comprising:
a. transducing a ProPreB cell with one or more vectors comprising a nucleic acid sequence encoding HOXB4; a nucleic acid sequence encoding PBX1, a nucleic acid sequence encoding LMO2; a nucleic acid sequence encoding ZFP467; and a nucleic acid sequence encoding ZFP521; wherein each said nucleic acid sequence is operably linked to a promoter; and
b. culturing the transduced ProPreB cell in a cell media that supports growth of ProPreB cells, thereby increasing survival and/or proliferation of ProPreB cells.

In some embodiments of these aspects and all such aspects described herein, the transducing of step (a) further comprises one or more vectors comprising one or more of: a nucleic acid sequence encoding KLF12; a nucleic acid sequence encoding HLF; and a nucleic acid sequence encoding EGR1.

Also provided herein, in some aspects, are isolated induced hematopoietic stem cells (iHSCs) produced using any of the HSC inducing compositions or methods described herein.

In some aspects, provided herein are cell clones comprising a plurality of the induced hematopoietic stem cells (iHSCs) produced using any of the HSC inducing compositions or methods described herein. In some embodiments of these aspects and all such aspects described herein, the cell clones further comprise a pharmaceutically acceptable carrier.

Also provided herein, in some aspects, are kits for making induced hematopoietic stem cells (iHSCs), the kits comprising any of the HSC inducing compositions comprising one or more expression vector components described herein.

Provided herein, in some aspects, are kits for making induced hematopoietic stem cells (iHSCs), the kits comprising any of the HSC inducing compositions comprising modified mRNA sequence components described herein.

In some embodiments of these aspects and all such aspects described herein, the at least one, two, three, four, or more HSC inducing factors are HLF, RUNX1T1, PBX1, LMO2, PRDM5, ZFP37, MYCN, and MEIS1

In some embodiments of these aspects and all such aspects described herein, the at least one, two, three, four, or more HSC inducing factors are HLF, RUNX1T1, ZFP37, PBX1, and LMO2.

Provided herein, in some aspects, are hematopoietic stem cell (HSC) inducing compositions comprising one or more expression vectors comprising:

a nucleic acid sequence encoding HLF;

a nucleic acid sequence encoding RUNX1T1;

a nucleic acid sequence encoding ZFP37;

a nucleic acid sequence encoding PBX1;

a nucleic acid sequence encoding LMO2;

a nucleic acid sequence encoding PRDM5;

a nucleic acid sequence encoding MYCN; and

a nucleic acid sequence encoding MEIS1.

Provided herein, in some aspects, are hematopoietic stem cell (HSC) inducing compositions comprising one or more expression vectors comprising:

a nucleic acid sequence encoding HLF;

a nucleic acid sequence encoding RUNX1T1;

a nucleic acid sequence encoding ZFP37;

a nucleic acid sequence encoding PBX1; and

a nucleic acid sequence encoding LMO2;

In some embodiments of these aspects and all such aspects described herein, the one or more expression vectors are lentiviral vectors. In some embodiments, the lentiviral vectors are inducible lentiviral vectors. In some embodiments, the lentiviral vectors are polycistronic inducible lentiviral vectors. In some embodiments, the polycistronic inducible lentiviral vectors express three or more nucleic acid sequences. In some embodiments, each of the nucleic acid sequences of the polycistronic inducible lentiviral vectors are separated by 2A peptide sequences.

In some embodiments of these aspects and all such aspects described herein, the at least one, two, three, four, or more HSC inducing factors are HLF, RUNX1T1, PBX1, LMO2, PRDM5, ZFP37, MYCN, and MEIS1.

In some embodiments of these aspects and all such aspects described herein, the at least one, two, three, four, or more HSC inducing factors are HLF, RUNX1T1, ZFP37, PBX1, and LMO2.

Provided herein, in some aspects, are hematopoietic stem cell (HSC) inducing compositions comprising: a modified mRNA sequence encoding HLF; a modified mRNA sequence encoding RUNX1T1; a modified mRNA sequence encoding ZFP37; a modified mRNA sequence encoding PBX1; a modified mRNA sequence encoding LMO2; a modified mRNA sequence encoding PRDM5; a modified mRNA sequence encoding MEIS1; and a modified mRNA sequence encoding MYCN; wherein each cytosine of each said modified mRNA sequence is a modified cytosine, each uracil of each said modified mRNA sequence is a modified uracil, or a combination thereof.

Provided herein, in some aspects, are hematopoietic stem cell (HSC) inducing compositions comprising a modified mRNA sequence encoding HLF; a modified mRNA sequence encoding RUNX1T1; a modified mRNA sequence encoding ZFP37; a modified mRNA sequence encoding PBX1; and a modified mRNA sequence encoding LMO2; wherein each cytosine of each said modified mRNA sequence is a modified cytosine, each uracil of each said modified mRNA sequence is a modified uracil, or a combination thereof.

Provided herein in some aspects, are methods for preparing an induced hematopoietic stem cell (iHSC) from a somatic cell comprising: transducing the somatic cell with one or more vectors comprising a nucleic acid sequence encoding HLF, a nucleic acid sequence encoding RUNX1T1; a nucleic acid sequence encoding ZFP37; a nucleic acid sequence encoding PBX1; a nucleic acid sequence encoding LMO2; and a nucleic acid sequence encoding PRDM5; a nucleic acid sequence encoding MEIS1; and a nucleic acid sequence encoding MYCN, wherein each said nucleic acid sequence is operably linked to a promoter; and

culturing the transduced somatic cell in a cell media that supports growth of hematopoietic stem cells, thereby preparing an iHSC.

Provided herein in some aspects, are methods for preparing an induced hematopoietic stem cell (iHSC) from a somatic cell comprising: transducing the somatic cell with one or more vectors comprising a nucleic acid sequence encoding HLF; a nucleic acid sequence encoding RUNX1T1; a nucleic acid sequence encoding ZFP37; a nucleic acid sequence encoding PBX1; and a nucleic acid sequence encoding LMO2; and a nucleic acid sequence encoding PRDM5, wherein each said nucleic acid sequence is operably linked to a promoter; and culturing the transduced somatic cell in a cell media that supports growth of hematopoietic stem cells, thereby preparing an iHSC.

As demonstrated herein, the use of polycistronic viral expression systems can increase the in vivo reprogramming efficiency of somatic cells to iHSCs. Accordingly, in some embodiments of the aspects described herein, a polycistronic lentiviral vector is used. In such embodiments, sequences encoding two or more of the HSC inducing factors described herein, are expressed from a single promoter, as a polycistronic transcript. We used 2A peptide strategy to make polycistronic vectors (see, e.g., Expert Opin Biol Ther. 2005 May; 5(5):627-38). Polycistronic expression vector systems can also use internal ribosome entry sites (IRES) elements to create multigene, or polycistronic, messages. IRES elements are able to bypass the ribosome scanning model of 5′-methylated Cap dependent translation and begin translation at internal sites (Pelletier and Sonenberg, 1988). IRES elements can be linked to heterologous open reading frames. Multiple open reading frames can be transcribed together, each separated by an IRES, thus creating polycistronic messages. By virtue of the IRES element, each open reading frame is accessible to ribosomes for efficient translation. Multiple genes can be efficiently expressed using a single promoter/enhancer to transcribe a single message. See, for example, U.S. Pat. Nos. 4,980,285; 5,925,565; 5,631,150; 5,707,828; 5,759,828; 5,888,783; 5,919,670; and 5,935,819; and Sambrook et al., Molecular Cloning: A Laboratory Manual, 2nd Ed., Cold Spring Harbor Press (1989).

### DEFINITIONS

For convenience, certain terms employed herein, in the specification, examples and appended claims are collected here. Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs.

The term “HSC inducing factor,” as used herein, refers to a developmental potential altering factor, as that term is defined herein, such as a protein, RNA, or small molecule, the expression of which contributes to the reprogramming of a cell, e.g. a somatic cell, to the HSC state. An HSC inducing factor can be, for example, transcription factors that can reprogram cells to the HSC state, such as HLF, RUNX1T1, PBX1, LMO2, PRDM5, ZFP37, MYCN, MSI2, NKX2-3, MEIS1, and RBPMS, and the like, including any gene, protein, RNA or small molecule that can substitute for one or more of these factors in a method of making iHSCs in vitro. In some embodiments, exogenous expression of an HSC inducing factor induces endogenous expression of one or more HSC inducing factors, such that exogenous expression of the one or more HSC inducing factor is no longer required for stable maintenance of the cell in the iHSC state.

As used herein, the terms “developmental potential” or “developmental potency” refer to the total of all developmental cell fates or cell types that can be achieved by a given cell upon differentiation. Thus, a cell with greater or higher developmental potential can differentiate into a greater variety of different cell types than a cell having a lower or decreased developmental potential. The developmental potential of a cell can range from the highest developmental potential of a totipotent cell, which, in addition to being able to give rise to all the cells of an organism, can give rise to extra-embryonic tissues; to a “unipotent cell,” which has the capacity to differentiate into only one type of tissue or cell type, but has the property of self-renewal, as described herein; to a “terminally differentiated cell,” which has the lowest developmental potential. A cell with “parental developmental potential” refers to a cell having the developmental potential of the parent cell that gave rise to it.

The term “multipotent” when used in reference to a “multipotent cell” refers to a cell that has the developmental potential to differentiate into cells of one or more germ layers, but not all three. Thus, a multipotent cell can also be termed a “partially differentiated cell.” Multipotent cells are well known in the art, and examples of multipotent cells include adult stem cells, such as for example, hematopoietic stem cells and neural stem cells. “Multipotent” indicates that a cell may form many types of cells in a given lineage, but not cells of other lineages. For example, a multipotent hematopoietic cell can form all of the many different types of blood cells (red, white, platelets, etc. . . . ), but it cannot form neurons. Accordingly, the term “multipotency” refers to a state of a cell with a degree of developmental potential that is less than totipotent and pluripotent.

The terms “stem cell” or “undifferentiated cell” as used herein, refer to a cell in an undifferentiated or partially differentiated state that has the property of self-renewal and has the developmental potential to differentiate into multiple cell types, without a specific implied meaning regarding developmental potential (i.e., totipotent, pluripotent, multipotent, etc.). A stem cell is capable of proliferation and giving rise to more such stem cells while maintaining its developmental potential. In theory, self-renewal can occur by either of two major mechanisms. Stem cells can divide asymmetrically, which is known as obligatory asymmetrical differentiation, with one daughter cell retaining the developmental potential of the parent stem cell and the other daughter cell expressing some distinct other specific function, phenotype and/or developmental potential from the parent cell. The daughter cells themselves can be induced to proliferate and produce progeny that subsequently differentiate into one or more mature cell types, while also retaining one or more cells with parental developmental potential. A differentiated cell may derive from a multipotent cell, which itself is derived from a multipotent cell, and so on. While each of these multipotent cells can be considered stem cells, the range of cell types each such stem cell can give rise to, i.e., their developmental potential, can vary considerably. Alternatively, some of the stem cells in a population can divide symmetrically into two stem cells, known as stochastic differentiation, thus maintaining some stem cells in the population as a whole, while other cells in the population give rise to differentiated progeny only. Accordingly, the term “stem cell” refers to any subset of cells that have the developmental potential, under particular circumstances, to differentiate to a more specialized or differentiated phenotype, and which retain the capacity, under certain circumstances, to proliferate without substantially differentiating. In some embodiments, the term stem cell refers generally to a naturally occurring parent cell whose descendants (progeny cells) specialize, often in different directions, by differentiation, e.g., by acquiring completely individual characters, as occurs in progressive diversification of embryonic cells and tissues. Some differentiated cells also have the capacity to give rise to cells of greater developmental potential. Such capacity may be natural or may be induced artificially upon treatment with various factors. Cells that begin as stem cells might proceed toward a differentiated phenotype, but then can be induced to “reverse” and re-express the stem cell phenotype, a term often referred to as “dedifferentiation” or “reprogramming” or “retrodifferentiation” by persons of ordinary skill in the art, and as used herein.

In the context of cell ontogeny, the term “differentiate”, or “differentiating” is a relative term that refers to a developmental process by which a cell has progressed further down a developmental pathway than its immediate precursor cell. Thus in some embodiments, a reprogrammed cell as the term is defined herein, can differentiate to a lineage-restricted precursor cell (such as a common lymphoid progenitor), which in turn can differentiate into other types of precursor cells further down the pathway (such as a ProBPreB cell, for example), and then to an end-stage differentiated cells, which play a characteristic role in a certain tissue type, and may or may not retain the capacity to proliferate further.

“Transdifferentiation,” as used herein refers to a process by which the phenotype of a cell can be switched to that of another cell type, without the formation of a multipotent intermediate cell. Thus, when transdifferentiation methods are employed, it is not required that the cell first be de-differentiated (or reprogrammed) to a multipotent cell and then differentiated to another hematopoietic lineage cell; rather the cell type is merely “switched” from one cell type to another without first forming a multipotent iHSC phenotype, for example.

As used herein, the term “without the formation of a multipotent or pluripotent intermediate cell” refers to the transdifferentiation of one cell type to another cell type, preferably, in one step; thus a method that modifies the differentiated phenotype or developmental potential of a cell without the formation of a multipotent or pluripotent intermediate cell does not require that the cell be first dedifferentiated (or reprogrammed) to a multipotent state and then differentiated to another cell type.

The term “expression” refers to the cellular processes involved in producing RNA and proteins and as appropriate, secreting proteins, including where applicable, but not limited to, for example, transcription, translation, folding, modification and processing. “Expression products” include RNA transcribed from a gene, and polypeptides obtained by translation of mRNA transcribed from a gene. In some embodiments, an expression product is transcribed from a sequence that does not encode a polypeptide, such as a microRNA.

As used herein, the term “transcription factor” or “TF” refers to a protein that binds to specific parts of DNA using DNA binding domains and is part of the system that controls the transcription of genetic information from DNA to RNA.

As used herein, the term “small molecule” refers to a chemical agent which can include, but is not limited to, a peptide, a peptidomimetic, an amino acid, an amino acid analog, a polynucleotide, a polynucleotide analog, an aptamer, a nucleotide, a nucleotide analog, an organic or inorganic compound (e.g., including heterorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

The term “exogenous” as used herein refers to a nucleic acid (e.g., a synthetic, modified RNA encoding a transcription factor), or a protein (e.g., a transcription factor) that has been introduced by a process involving the hand of man into a biological system such as a cell or organism in which it is not normally found, or in which it is found in lower amounts. A factor (e.g. a synthetic, modified RNA encoding a transcription factor, or a protein, e.g., a polypeptide) is considered exogenous if it is introduced into an immediate precursor cell or a progeny cell that inherits the substance. In contrast, the term “endogenous” refers to a factor or expression product that is native to the biological system or cell (e.g., endogenous expression of a gene, such as, e.g., HLF refers to production of an HLF polypeptide by the endogenous gene in a cell).

The term “isolated” or “partially purified” as used herein refers, in the case of a nucleic acid or polypeptide, to a nucleic acid or polypeptide separated from at least one other component (e.g., nucleic acid or polypeptide) that is present with the nucleic acid or polypeptide as found in its natural source and/or that would be present with the nucleic acid or polypeptide when expressed by a cell, or secreted in the case of secreted polypeptides. A chemically synthesized nucleic acid or polypeptide or one synthesized using in vitro transcription/translation is considered “isolated”.

The term “isolated cell” as used herein refers to a cell that has been removed from an organism in which it was originally found, or a descendant of such a cell. Optionally the cell has been cultured in vitro, e.g., in the presence of other cells. Optionally, the cell is later introduced into a second organism or re-introduced into the organism from which it (or the cell or population of cells from which it descended) was isolated.

The term “isolated population” with respect to an isolated population of cells as used herein refers to a population of cells that has been removed and separated from a mixed or heterogeneous population of cells. In some embodiments, an isolated population is a “substantially pure” population of cells as compared to the heterogeneous population from which the cells were isolated or enriched. In some embodiments, the isolated population is an isolated population of multipotent cells which comprise a substantially pure population of multipotent cells as compared to a heterogeneous population of somatic cells from which the multipotent cells were derived.

The term “immediate precursor cell” is used herein to refer to a parental cell from which a daughter cell has arisen by cell division.

The term “contacting” or “contact” as used herein in connection with contacting a cell with one or more constructs, viral vectors, or synthetic, modified RNAs, includes subjecting a cell to a culture medium which comprises one or more constructs, viral vectors, or synthetic, modified RNAs at least one time, or a plurality of times, or to a method whereby such constructs, viral vectors, or synthetic, modified RNAs are forced to contact a cell at least one time, or a plurality of times, i.e., a transduction or a transfection system. Where such a cell is in vivo, contacting the cell with a construct, viral vector, or synthetic, modified RNA includes administering the construct(s), viral vector(s), or synthetic, modified RNA(s) in a composition, such as a pharmaceutical composition, to a subject via an appropriate administration route, such that the compound contacts the cell in vivo.

The term “transfection” as used herein refers the use of methods, such as chemical methods, to introduce exogenous nucleic acids, such as synthetic, modified RNAs, into a cell, preferably a eukaryotic cell. As used herein, the term transfection does not encompass viral-based methods of introducing exogenous nucleic acids into a cell. Methods of transfection include physical treatments (electroporation, nanoparticles, magnetofection), and chemical-based transfection methods. Chemical-based transfection methods include, but are not limited to, cyclodextrin, polymers, liposomes, and nanoparticles. In some embodiments, cationic lipids or mixtures thereof can be used to transfect the synthetic, modified RNAs described herein, into a cell, such as DOPA, Lipofectamine and UptiFectin. In some embodiments, cationic polymers such as DEAE-dextran or polyethylenimine, can be used to transfect a synthetic, modified RNAs described herein.

The term “transduction” as used herein refers to the use of viral particles or viruses to introduce exogenous nucleic acids, such as nucleic acid sequences encoding HSC inducing factors, into a cell.

As used herein, the term “transfection reagent” refers to any agent that induces uptake of a nucleic acid into a host cell. Also encompassed are agents that enhance uptake e.g., by at least 10%, at least 20%, at least 30%, at least 40%, at least 50%, at least 60%, at least 70%, at least 80%, at least 90%, at least 95%, at least 99%, at least 1-fold, at least 2-fold, at least 5-fold, at least 10-fold, at least 25-fold, at least 500-fold, at least 100-fold, at least 1000-fold, or more, compared to a nucleic acid sequence administered in the absence of such a reagent. In some embodiments, a cationic or non-cationic lipid molecule useful for preparing a composition or for co-administration with a synthetic, modified RNA is used as a transfection reagent. In other embodiments, the synthetic, modified RNA comprises a chemical linkage to attach e.g., a ligand, a peptide group, a lipophilic group, a targeting moiety etc. In other embodiments, the transfection reagent comprises a charged lipid, an emulsion, a liposome, a cationic or non-cationic lipid, an anionic lipid, or a penetration enhancer as known in the art or described herein.

As used herein, the term “repeated transfections” refers to repeated transfection of the same cell culture with a nucleic acid, such as a synthetic, modified RNA, a plurality of times (e.g., more than once or at least twice). In some embodiments, the cell culture is transfected at least twice, at least 3 times, at least 4 times, at least 5 times, at least 6 times, at least 7 times, at least 8 times, at least 9 times, at least 10 times, at least 11 times, at least 12 times, at least 13 times, at least 14 times, at least 15 times, at least 16 times, at least 17 times at least 18 times, at least 19 times, at least 20 times, at least 25 times, at least 30 times, at least 35 times, at least 40 times, at least 45 times, at least 50 times or more. The transfections can be repeated until a desired phenotype of the cell is achieved.

The time between each repeated transfection is referred to herein as the “frequency of transfection.” In some embodiments, the frequency of transfection occurs every 6 h, every 12 h, every 24 h, every 36 h, every 48 h, every 60 h, every 72 h, every 96 h, every 108 h, every 5 days, every 7 days, every 10 days, every 14 days, every 3 weeks, or more during a given time period in any developmental potential altering regimen. The frequency can also vary, such that the interval between each dose is different (e.g., first interval 36 h, second interval 48 h, third interval 72 h etc). It should be understood depending upon the schedule and duration of repeated transfections, it will often be necessary to split or passage cells or change or replace the media during the transfection regimen to prevent overgrowth and replace nutrients. For the purposes of the methods described herein, transfections of a culture resulting from passaging an earlier transfected culture is considered “repeated transfection,” “repeated contacting” or “contacting a plurality of times,” unless specifically indicated otherwise.

As used herein, the terms “nucleic acid,” “polynucleotide,” and “oligonucleotide” generally refer to any polyribonucleotide or poly-deoxyribonucleotide, and includes unmodified RNA, unmodified DNA, modified RNA, and modified DNA. Polynucleotides include, without limitation, single- and double-stranded DNA and RNA polynucleotides. The term polynucleotide, as it is used herein, embraces chemically, enzymatically or metabolically modified forms of polynucleotides, as well as the naturally occurring chemical forms of DNA and RNA found in or characteristic of viruses and cells, including for example, simple (prokaryotic) and complex (eukaryotic) cells. A nucleic acid polynucleotide or oligonucleotide as described herein retains the ability to hybridize to its cognate complimentary strand.

Accordingly, as used herein, the terms “nucleic acid,” “polynucleotide,” and “oligonucleotide” also encompass primers and probes, as well as oligonucleotide fragments, and is generic to polydeoxyribonucleotides (containing 2-deoxy-D-ribose), to polyribonucleotides (containing D-ribose), and to any other type of polynucleotide which is an N-glycoside of a purine or pyrimidine base, or modified purine or pyrimidine bases (including, but not limited to, abasic sites). There is no intended distinction in length between the term “nucleic acid,” “polynucleotide,” and “oligonucleotide,” and these terms are used interchangeably. These terms refer only to the primary structure of the molecule. An oligonucleotide is not necessarily physically derived from any existing or natural sequence, but can be generated in any manner, including chemical synthesis, DNA replication, DNA amplification, in vitro transcription, reverse transcription or any combination thereof

The terms “nucleotide” or “mononucleotide,” as used herein, refer to a phosphate ester of a nucleoside, e.g., mono-, di-, tri-, and tetraphosphate esters, wherein the most common site of esterification is the hydroxyl group attached to the C-5 position of the pentose (or equivalent position of a non-pentose “sugar moiety”). The term “nucleotide” includes both a conventional nucleotide and a non-conventional nucleotide which includes, but is not limited to, phosphorothioate, phosphite, ring atom modified derivatives, and the like.

As used herein, the term “conventional nucleotide” refers to one of the “naturally occurring” deoxynucleotides (dNTPs), including dATP, dTTP (or TTP), dCTP, dGTP, dUTP, and dITP.

As used herein, the term “non-conventional nucleotide” refers to a nucleotide that is not a naturally occurring nucleotide. The term “naturally occurring” refers to a nucleotide that exists in nature without human intervention. In contradistinction, the term “non-conventional nucleotide” refers to a nucleotide that exists only with human intervention, i.e., an “artificial nucleotide.” A “non-conventional nucleotide” can include a nucleotide in which the pentose sugar and/or one or more of the phosphate esters is replaced with a respective analog. Exemplary phosphate ester analogs include, but are not limited to, alkylphosphonates, methylphosphonates, phosphoramidates, phosphotriesters, phosphorothioates, phosphorodithioates, phosphoroselenoates, phosphorodiselenoates, phosphoroanilothioates, phosphoroanilidates, phosphoroamidates, boronophosphates, etc., including any associated counterions, if present. A non-conventional nucleotide can show a preference of base pairing with another non-conventional or “artificial” nucleotide over a conventional nucleotide (e.g., as described in Ohtsuki et al. 2001, Proc. Natl. Acad. Sci., 98: 4922-4925, hereby incorporated by reference). The base pairing ability may be measured by the T7 transcription assay as described in Ohtsuki et al. (supra). Other non-limiting examples of “non-conventional” or “artificial” nucleotides can be found in Lutz et al. (1998) Bioorg. Med. Chem. Lett., 8: 1149-1152); Voegel and Benner (1996) Helv. Chim Acta 76, 1863-1880; Horlacher et al. (1995) Proc. Natl. Acad. Sci., 92: 6329-6333; Switzer et al. (1993), Biochemistry 32:10489-10496; Tor and Dervan (1993) J. Am. Chem. Soc. 115: 4461-4467; Piccirilli et al. (1991) Biochemistry 30: 10350-10356; Switzer et al. (1989) J. Am. Chem. Soc. 111: 8322-8323, all of which are hereby incorporated by reference. A “non-conventional nucleotide” can also be a degenerate nucleotide or an intrinsically fluorescent nucleotide.

As used herein the term “modified ribonucleoside” refers to a ribonucleoside that encompasses modification(s) relative to the standard guanine (G), adenine (A), cytosine (C), and uracil (U) nucleosides. Such modifications can include, for example, modifications normally introduced post-transcriptionally to mammalian cell mRNA, and artificial chemical modifications, as known to one of skill in the art.

As used herein, the terms “synthetic, modified RNA” or “modified RNA” or “modified mRNA” refer to an RNA molecule produced in vitro which comprises at least one modified nucleoside as that term is defined herein below. The modified mRNAs do not encompass mRNAs that are isolated from natural sources such as cells, tissue, organs etc., having those modifications, but rather only synthetic, modified RNAs that are synthesized using in vitro techniques, as described herein. The term “composition,” as applied to the terms “synthetic, modified RNA” or “modified RNA,” encompasses a plurality of different synthetic, modified RNA molecules (e.g., at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 40, at least 50, at least 75, at least 90, at least 100 synthetic, modified RNA molecules or more). In some embodiments, a synthetic, modified RNA composition can further comprise other agents (e.g., an inhibitor of interferon expression or activity, a transfection reagent, etc.). Such a plurality can include synthetic, modified RNA of different sequences (e.g., coding for different polypeptides), synthetic, modified RNAs of the same sequence with differing modifications, or any combination thereof.

As used herein the term “modified nucleoside” refers to a ribonucleoside that encompasses modification(s) relative to the standard guanine (G), adenine (A), cytidine (C), and uridine (U) nucleosides. Such modifications can include, for example, modifications normally introduced post-transcriptionally to mammalian cell mRNA, and artificial chemical modifications, as known to one of skill in the art.

As used herein, the term “polypeptide” refers to a polymer of amino acids comprising at least 2 amino acids (e.g., at least 5, at least 10, at least 20, at least 30, at least 40, at least 50, at least 60, at least 70, at least 80, at least 90, at least 100, at least 125, at least 150, at least 175, at least 200, at least 225, at least 250, at least 275, at least 300, at least 350, at least 400, at least 450, at least 500, at least 600, at least 700, at least 800, at least 900, at least 1000, at least 2000, at least 3000, at least 4000, at least 5000, at least 6000, at least 7000, at least 8000, at least 9000, at least 10,000 amino acids or more). The terms “protein” and “polypeptide” are used interchangeably herein. As used herein, the term “peptide” refers to a relatively short polypeptide, typically between about 2 and 60 amino acids in length.

### BRIEF DESCRIPTION OF THE DRAWINGS

FIG. 1 depicts a schematic of hematopoietic differentiation showing populations (boxes) for which microarray data has been generated. Data generated herein is shown in thin-line boxes, and by other groups in thick-line boxes. Whereas hematopoietic differentiation normally proceeds from HSCs to differentiated blood effector cells, the results described herein aim to utilize HSC-enriched transcription factors to reprogram committed hematopoietic cells back to HSCs (large arrow). Throughout this proposal HSCs are purified by stringent cell surface criteria (e.g., ckit⁺Sca1⁺lineage⁻CD48⁻flk2⁻CD150⁺CD34⁻), as well as for fetal liver HSCs (e.g., ckit⁺Sca1⁺lineage−CD48⁻CD150⁺Mac1^{low}).

FIG. 2 depicts an overview of the approaches described herein for identifying factors capable of reprogramming committed hematopoietic cells back to HSCs.

FIG. 3 depicts gene discovery using the hematopoietic expression database. Heat map of expression of genes enriched in 6 different hematopoietic populations. Each column reflects microarray data from a hematopoietic subset (40 populations represented). Erythroid progenitors include MEP, pre-CFU-E and CFU-E. Expressed was visualized as red; Not expressed was visualized as blue. * Asterisk denotes genes with known roles in specifying the fate and/or function of the indicated cell type.

FIGS. 4A-4B depict an overview of experimental approaches and experimental populations. FIG. 4A depicts experimental approaches for screening induced HSCs (iHSCs) through expression of multiple critical HSC-enriched transcription factors by in vitro and in vivo methods. CD45.2 transgenic (rtTA) mice are used to identify congenic donor cells in transplant experiments using recipient CD45.1 host mice. Common myeloid progenitors (CMPs) and Pro/Pre B Cells were sorted out of the bone marrow of CD45.2 transgenic mice. Sorted cells were incubated for 14 hours with ZsGreen control (VC) or a viral cocktail of HSC-specific factors. ZsGr+ cells were resorted two days post doxycycline addition. Resorted ZsGr+ CMPs and ProPreB Cells were put into a CFC myeloid colony forming assays (scored for colony numbers and morphology 20 days later) or transplanted into conditioned IR CD45.1+ recipient mice. Peripheral bleeds were performed up to 16 weeks as to define the short and long term reconstitution potential of cells. Mice identified with adequate multi-lineage reconstitution were euthanized and donor derived cells sorted from the bone marrow to be transplanted into conditioned secondary CD45.1 recipients; also full analysis of the bone marrow, spleen and thymus was performed. FIG. 4B depict CMPs and PrePro B cells that were predominately chosen as our starting populations so that we could demonstrate experimental reprogramming from the first defined committed blood cells in BOTH the B cell lineage and the myeloid lineage. These cell populations were identified using the phenotypic markers listed.

FIGS. 5A-5C depict heat maps of HSC-enriched transcription factors. The Rossi Lab and others put together a detailed database including mRNA expression profiles for over 248 defined progenitor and effector sub populations. FIG. 5A depicts an expression profile heat map for 37 HSC-enriched reprogramming factors. Columns represent microarray data for 40 distinct FACs sorted populations. * Denotes factors chosen because of their developmental importance. Expressed was visualized as red; Not expressed was visualized as blue. FIG. 5B shows that all HSC-enriched factors were placed into a doxycycline inducible tet-on system based in the pHAGE2 lentiviral vector. Only exception to this vector map from addgene is that a CMV promoter is used in the systems described herein. Heat Map of expanded set of identified HSC-enriched Transcription Factors. FIG. 5C depicts an expression profile heat map for 46 HSC-enriched putative reprogramming factors. Columns represent microarray data for 40 distinct FACs sorted populations. * Expressed was visualized as red; Not expressed was visualized as blue.

FIGS. 6A-6D depict isolation strategies for Pro and Pre B cells. FIG. 6A shows ProPre B cells that are sorted from the bone marrow by placing total bone marrow through a magnetic B220 enrichment column. Enrichment increases B220⁺CD19⁺ B cells from 15% to 85% in their respective populations; through Aria cell sorting the purity of the sample increases further to 99-100%. (RT stands for the B220⁻ run through from the column) FIG. 6B depicts a orting strategy to obtain ProPreB Cells that is demonstrated by flow histograms. FIG. 6C shows overall purity for each of the following samples: overall B220 enriched (top panel), reanalyzed sorted Pro B cells (Middle panel) and reanalyzed sorted Pre B cells (Bottom Panel). By showing CD25 expression vs. B220 expression we demonstrate not only that Pro and Pre B cells can be effectively sorted but can also be distinguished via phenotypic markers and sorting. FIG. 6D depicts overall sort purity of Pre B cells and Pro B Cells in each of the populations collected; indicating proficient sorting of ProPre B Cells (RT stands for the B220⁻ run through from the column).

FIGS. 7A-7B depict an isolation strategy for CMPs. FIG. 7A shows CMP cells that are sorted from the bone marrow by placing total bone marrow through a magnetic c-kit enrichment column. The indicated gating strategy isolated singlet, live, lineage negative, hematopoietic progenitors. FIG. 7B shows that enrichment increases CMP levels and furthermore that using aria cell sorting, a purity of 99-100% is achieved.

FIGS. 8A-8C demonstrate transduction and inducible expression of HSC-enriched transcription factors (TFs) in hematopoietic progenitors. FIG. 8A shows transduction of multi-potent progenitors (MPPs) with lentiviruses bearing 8 different TFs (LV1-LV-8). Cells were cultured in the presence of doxycycline (Dox) for 5 days followed by flow cytometry. FIG. 8B shows peripheral blood of a recipient transplanted with TF-transduced MPPs and maintained on Dox for 4 weeks (left panel), followed by 2 weeks Dox-off (right panel). FIG. 8C shows viral mediated expression of putative reprogramming factors in vitro. Quantitative RT-PCR for the indicated genes showing their relative expression within primary hematopoietic stem cells (HSCs) or multi-potent progenitors (MPPs), and in primary cells that were transduced with LV encoding the indicated factor and cultured for 1 week. The mRNA levels in overexpressing cells was calculated by dividing to the expression levels in primary HSCs. Results show Hlf at 8-fold, Nap113 at 110-fold, Rbpms at 20-fold and Runx1′ at 40-fold above endogenous levels.

FIGS. 9A-9C demonstrate that Pro/Pre B Cells and CMPs can be transduced with doxycycline inducible viral cocktails. FIG. 9A shows B220+ CD19+ B Cells that were sorted from the bone marrow; cells were incubated for 14 hours with nothing (non trans), control ZsGr Virus (VC) or a viral cocktail that express 28 HSC-enriched factors (VM). Doxycycline (dox) was added for 24 hours. An increase in ZsGr+ cells is observed when the VM is used on cells in comparison to non transduced cells. FIG. 9B shows B220+ CD19+ B cells that were further analyzed in the presence and absence of dox in three independent trials. In the absence of Dox few ZsGr+ cells are observed however regardless of using VC or VM the addition of Dox increases ZsGr expression in the population. Addition of dox tightly regulates ZsGr expression and therein gene expression. FIG. 9C shows pre B Cells, Pro B Cells, and CMPs that were sorted out of the bone marrow and incubated for 14 hours with VC or VM and left with Dox for two days before analysis. ProPreBCells and CMPs can be transduced with the viral cocktail to express HSC-enriched factors.

FIGS. 10A-10D demonstrate that combinatorial TF expression increases ProPreB and CMP CFC colony number and alters lineage potential. ProPre B Cells and CMPs were sorted using phenotypic markers on the Aria Sorter. Cells were incubated with ZsGr control virus (VC) or a viral cocktail (VM) for 14 hours in S-clone media containing SCF, TPO and IL-12 (In the case of ProPreB Cells, IL-7 and Flk3). Dox was added for 24 hours and cells were resorted for ZsGr+ cells. ZsGr+ cells were placed into methylcellulose media in a 6 well plate format containing SCF, TPO and IL-12 (For ProPreB Cells IL-7 and Flk3). Colony forming potential was assayed on day 20. FIG. 10A shows examples of types of cells observed during determination of colony morphology. FIG. 10B depicts representative pictures that were taken of the Transduced ProPreB ZsGreen control (VC) and Viral mixture of 37 factors (VM) CFC plates. FIG. 10C shows increasing number of cells that were plated to find an effective plating density of both ProPreBCells and CMPs. 2×10⁵ ProPre B Cells and 1×10⁴ CMPs were used in further experiments. Experiments were repeated in two individual trials. FIG. 10D shows colony number and composition that were determined and noted for all colonies. Increased colony number is observed when ProPreB Cells and CMPs were transduced with the cocktail of 37 factors as compared to the ZsGreen control (VC). Experiments were done in duplicates for four trials.

FIG. 11 demonstrates that exposure to 18 putative reprogramming factors embues multi-potent progenitors with robust long-term multi-lineage engraftment potential in vivo. Multi-potent progenitors (MPP=Lineage⁻Sca1⁺ckit⁺CD150⁻) were sorted and transduced with either control virus of a lentiviral mix containing Hlf, MycN, Meis1, Irf6, Cdkn1c, Nfix, Dnmt3b, Zfp612, Prdm5, HoxB4, Lmo2, Nkx2-3, RarB, Ndn, Nap113, Runx1t1, Zfp467, Zfp532. Transduced cells were transplanted into irradiated congenic recipients along with competitive WBM. Peripheral-blood chimerism is indicated at timepoints post-transplant showing that exposure to these factors greatly improved long-term donor engraftment.

FIG. 12 demonstrates that exposure to 9 putative reprogramming factors embues multi-potent progenitors with robust long-term multi-lineage engraftment potential in vivo. MPPs from CD45.2 or congenic CD45.1 donors were sorted as LSKCD34+flk2+ and equal numbers of cells were transduced with either control virus (into CD45.1 cells) of a lentiviral mix containing 9 factors, including Evi-1, Glis2, HoxB5, HoxA9, HLF, Meis1, MycN, Prdm16, Runx1 (CD45.2 cells). Cells were transplanted into irradiated CD45.1/CD45.2 F1 recipients along with CD45.1/CD45.2 competitor bone marrow (2e5 cells). Transgene-expression was sustained with doxycycline (dox-on) for 18 weeks (upper panel) followed by removal of Doxycycline for the remainder of the experiment (dox-off). Peripheral blood chimerism was measured at 20 and 25 weeks (lower panel) showing that in contrast to control transduced MPPs (CD45.1), 9-factor transduced MPPS retained rebust long-term repopulating activity. Panel on lower right: Engraftment from 9-factor transduction is multi-lineage. Donor-derived cells were stained for Mac1, Gr-1, CD3, CD8 and B220 revealing the presence of donor-derived, macrophage/monocytes, granulocytes, T-cells and B-cells.

FIGS. 13A-13B demonstrate long-term multi-lineage reconstitution of multi-potent progenitors (MPPs) transduced with HSC-enriched transcription factors (TFs). FIG. 13A. Flow cytometry of peripheral blood of a recipient transplanted with MPPs (ckit+Sca1+lineage−CD150−flk2+CD34+) transduced with control virus (top panel), or a cocktail of 17 different TFs (lower panel), 20 weeks post-transplant. Equal numbers of MPPs from the same initial sort were transplanted. FIG. 13B. Donor chimerism 20 weeks post-transplant of mice described in (FIG. 13A). Results show that only the TF-transduced MPPs yielded long-term multi-lineage reconstitution of T-cells, B-cells and myeloid cells, whereas control cells only gave rise to lymphoid cells as expected. All recipients receiving TF-transduced cells were multi-lineage reconstituted suggesting that reprogramming was not a rare event. n=4 recipients for each control and 17-TF. 17 factors in this experiment included: Hlf, MycN, Meis1, Irf6, Nfix, Dnmt3b, Zfp612, Prdm5, HoxB4, Lmo2, Nkx2-3, RarB, Ndn, Nap113, Runx1t1, Zfp467, Zfp532.

FIG. 14 demonstrates that exposure to 8 putative reprogramming factors embues multi-potent progenitors with robust long-term multi-lineage engraftment potential in vivo. Multi-potent progenitors (MPP=Lineage⁻Sca1⁺ckit⁺CD150⁻flk2⁺CD34⁺) were sorted and transduced with with either control virus of a lentiviral mix containing Runx1t1, HLF Zfp467 Rbpms hoxb5 nap113 msi2 Irf6. Transduced cells were transplanted into irradiated congenic recipients along with competitive WBM. Peripheral-blood chimerism is indicated at 16 weeks post-transplant showing that exposure to these factors led to long-term donor multi-lineage engraftment (bottom panel) in contrast to control transduced cells (top panel). Doxycline was maintained on for 2 weeks post-transplant followed by dox-removal.

FIG. 15 depicts using peripheral bleeds to test donor derived chimerism. Shown here is an example gating strategy on a peripheral bleeds done at 8 weeks on a transplanted mouse with ProPreB cells transduced with a cocktail of viruses that individually encode for expression of 37 transcription factors.

FIGS. 16A-16C demonstrate that ProPreB Cell transplantation confers multi-lineage peripheral reconstitution when factors are expressed combinatorially. CD45.2+ ProPreB cells and CMPs transduced with control or VM were transplanted competitively into IR CD45.1+ recipients. Peripheral bleeds were performed at 4, 8, 12, and 16 weeks. FIG. 16A. Flow histograms show 16 week peripheral bleeds for controls (VC—top panels) and cells expressing the mix of 37 factors (VM—bottom panels); demonstrated for ProPreB (Left) and CMP (Right). FIG. 16B. Quantitative results for each of the peripheral bleeds are shown for ProPreB Cells and CMPs. Chimerism above 1.0% was observed in 5/14 mice transplanted with ProPreB and 3/8 mice transplanted with CMP. FIG. 16C. Cellular composition of the peripheral bleeds of mice with chimerism over 1.0% is shown for mice transplanted with ProPreB Cells and CMPs.

FIG. 17 demonstrates that peripheral lymphoid organ and bone marrow reconstitution is observed from CMPs and ProPreB Cells expressing combinatorial factors. The bone marrow, spleen, and thymus were harvested from mice transplanted with ProPreB Cells/CMPs transduced with control (VC) a viral cocktail (VM). Representative histograms of three ProPre B Cell transplanted mice (VC, VM4, VM14) and two CMP transplanted mice (VC and VM6)—VM#s are the same observed in FIG. 15. Varying degrees of donor derived chimerism can be observed in each lymphoid compartment; consistently VM expressing cells had higher reconstitution in all lymphoid compartments in comparison to controls.

FIGS. 18A-18D demonstrate that multi-lineage reconstitution is observed in peripheral lymphoid organs upon transplantation with combinatorial factor expression. FIG. 18A. The bone marrow, spleen, and thymus were harvested from mice that were transplanted with transduced ProPre B cells and CMPs. Quantitation of the data is graphically summarized. In all ProPreB cells transplanted mice with >1.0% peripheral blood chimerism, donor derived chimerism above control levels were observed in all lymphoid compartments analyzed. FIGS. 18B-18D. Composition of the bone marrow, spleen, and thymus for all control mice or experimental mice analyzed with >1% peripheral blood chimerism.

FIGS. 19A-19D demonstrate that ProPreB Cells and CMPs expressing a cocktail of factors give rise to primitive hematopoietic progenitors. FIG. 19A. Flow plots have been previously gated on myeloid progenitors (top panel) or primitive hematopoietic progenitors (LSK (Lin⁻Sca⁺c-kit⁺) cells) (bottom panel). Only mice that received cells transduced with the viral cocktail give rise to donor (CD45.2+) derived cells hematopoietic progenitors or myeloid progenitors. Further break down of the myeloid progenitor gate (top panel) and hematopoietic progenitor (bottom panel) gates reveal a diversity of progenitor populations. FIG. 19B. Quantitation of the overall numbers of myeloid progenitors and hematopoietic progenitor cells in each of the transplanted VC (average of five mice) and VM mice with peripheral chimerism above 1.0%. In all cases there is increased numbers of cells with respect to controls. FIGS. 19C-19D. Composition of the compartments was analyzed and quantified. Each bar represents one mouse and the respective composition of the myeloid progenitor compartment (FIG. 19C) or the hematopoietic progenitor compartment (FIG. 19D).

FIGS. 20A-20C demonstrate that ProPre B Cells and CMPs have serial transplant potential only when factors in combination are expressed. 1000 LSK CD45.2+ Cells were sorted and transplanted competitively with 2×105 CD45.1+ Competitors into competent CD45.1+ hosts. FIG. 20A. At 4 weeks all the secondary transplants had distinguishable donor derived multi-lineage populations. Flow graphs representing each of those secondary transplants are shown. FIG. 20B. Quantitation of these results was calculated and reported here as the % CD45.2+ of total peripheral blood. Only ProPre B Cell VM #14 had sustainable (>0.1%) long-term multi-lineage reconstitution even at 16 weeks. FIG. 20C. The composition of the peripheral blood for all the mice referred to above at four weeks and at 16 weeks for PPBC#14. Multi-lineage reconstitution is observed for all bleeds.

FIGS. 21A-21B. PCR based strategies can be used to identify VDJ rearrangements in B-cell progenitors. FIG. 21A. B cells progenitors can be isolated based on the phenotypic markers shown in this schematic. FIG. 21B. Fraction A, B, C and D and IgM positive mature B cells were sorted and subjected to PCR for V-D-J recombination of heavy and light chain. Heavy chain rearrangement begins as early as fraction B and continues to occur through Fraction C. Lambda and kappa light chain and rearrangement can occur as early as Fraction C and proceed through mature B cells. CD45.2 was used as a PCR loading control across all the samples. The experiments described herein demonstrate that we can effectively detect rearrangements in ProPreB Cells (Fractions B-D) in our system by PCR detection of rearrangement. Primers were adapted primers from Cobaleda et al. Nature 2007.

FIGS. 22A-22C demonstrate VDJ rearrangement confirms the B-lineage origin of reprogrammed cells. To determine if cell populations and colonies originated from a VDJ recombined cell we assayed for recombinational events using PCR. FIG. 22A. B cells (B220+), hematopoietic progenitor (Live, Lin−, c-kit+, Sca+), and myeloid progenitor (Live, Lin−, c-kit+, Sca−) bone marrow cells were FACs cell sorted and analyzed by PCR for heavy chain VDJ recombination. These populations provide a positive and two negative controls. Colonies arising from ProPreB cells expressing a mix of TFs were tested (GEMM colony); A myeloid colony taken from the control plate. FIG. 22B. CD45.2+ donor and CD45.1+ recipient Mac1+ cells were FACs sorted. PCR was performed to test heavy chain (J_{H558}), kappa light chain (JLk), lambda light chain (JL1); genomic CD45 as a loading control. This demonstrates rearrangement in Mac+ cells isolated from a mouse transplanted with ProPreB Cells transduced with the viral cocktail (ProPreB #4). FIG. 22C. Recombination analysis was performed and is summarized in table format for mice with CD45.2+ chimerism >1.0%. All mice with donor derived chimerism and transplanted with ProPre B Cells transduced with the viral cocktail had evidence of reprogramming on the heavy chain loci; a majority had either lambda or kappa light chain rearrangement. All recombinational events appear to be polyclonal and therefore reconstitution occurred from multiple clones.

FIGS. 23A-23B demonstrate that VDJ Rearrangement confirms the origin of the reprogrammed cells. Although summarized in FIG. 22C, further per testing of recombinational events in the peripheral blood of mice reconstituted by ProPreB Cells transduced with the viral cocktail. FIG. 23A. Rearrangement PCR testing Mac1+ cells isolated from mice reconstituted with reprogrammed Pre/Pro B-cells (mice #'s 3, 7, 14) by a viral cocktail. B220+ cells are used as the positive control and primitive hematopoietic progenitors (unrearranged LSK cells) as the negative control. In the last lane is a mixed myeloid lineage CFC colony (GEMM) that was tested for both heavy and light chain rearrangement. FIG. 23B. Rearrangement of Mac1+ cells sorted from the peripheral blood of a mouse reconstituted with reprogrammed Pre/Pro B-cells (VM#5). B220+ cells isolated from the bone marrow (BM) and peripheral blood (PB) are used as the positive control; primitive hematopoietic progenitors (unrearranged LSK+ cells) as the negative control. In the last lane is a mixed myeloid lineage CFC colony (GEMM) that was tested for both heavy and light chain rearrangement.

FIG. 24 demonstrates that VDJ Rearrangement confirms the origins of peripheral blood cells. Although rearrangement was observed in Mac+ positive cells from the peripheral blood, further analysis was performed on other populations from mice reconstituted from transplanted ProPre B cells transduced with the viral cocktail (#3 and #4). From these two mice the following donor (CD45.2+) populations were sorted: CD4/8+ T cells (T), B220+ B Cells (B), Mac1+ Myeloid cells (M), and all other cells with none of those markers (N). Each population displayed evidence of B cell recombinational events.

FIGS. 25A-25D demonstrates that VDJ rearrangement confirms the origins of peripheral lymphoid cells and bone marrow populations. Tracking of VDJ B cell rearrangement in mice partially reconstituted by the proposed iHSC cells was taken one step further. When bone marrow of mice reconstituted from ProPreB cells transduced with the viral cocktail, aliquots of 50 cells were taken of donor derived hematopoietic progenitors [CD45.2+ LSK cells (LSK)], B cells [B220+ (B Cell)], myeloid cells [Mac1+ (Mac)], Myeloid progenitors [Lin−Sca−c-kit+=(MylPro)] and T cells [CD4+/8+/3+ T Cels (T cell)]. DNA was extracted from the samples and PCR performed to assay for recombination. FIG. 25A. PCR recombination testing of mouse (#4) reconstituted from ProPreB Cells transduced with the viral mix. PCR testing was performed for heavy chain (J_{H588}), kappa light chain (Jₖ), and lambda light chain (Jₗ). FIG. 25B. PCR recombination testing of mouse (#3) reconstituted from ProPreB Cells transduced with the viral mix. PCR testing was performed for heavy chain (J_{H588}). FIG. 25C. PCR recombination testing of mouse (#14 and #7) reconstituted from ProPreB Cells transduced with the viral mix. PCR testing was performed for heavy chain (JH588). For mouse #14 that had high donor derived chimerism additional analysis was performed on the same populations from the spleen. Recipient CD45.1+ cells were included as a negative control. FIG. 25D. PCR recombination testing of mouse (#7) reconstituted from ProPreB Cells transduced with the viral mix. PCR testing was performed for heavy chain (J_{H588}). Analysis of CD3/CD4/CD8+ T cells from the thymus. The left lane is CD45.1+ control T cells and the right is CD45.2+ donor cells. Only donor cells expressed B cell recombinational events.

FIG. 26 demonstrates a strategy for reverse cloning of reprogramming factors that allows for distinction between endogenous loci (top panel) and integrated reprogramming factors. Primers were designed to straddle intron/exon boundaries such that PCR identification of virally introduced transcription factors could readily be resolved from the endogenous genes—with the reprogramming factors yielding a smaller PCR product in all cases. See Table 5 for primer sequences used for reverse cloning of all reprogramming factors.

FIG. 27 demonstrates reverse cloning identification of transcription factors. ProPreB Cells were sorted and transduced for 14 hours with ZsGr control virus (VC), A single virus listed (Only Vector), a viral mix of 37 different factors minus that listed virus (VM-Vector) or the viral cocktail of 37 factors (VM). Doxycycline was added for 24 hours and then cells were harvested, DNA isolated, and PCR analysis performed using the indicated primers.

FIG. 28 shows reverse cloning identification of transcription factors. ProPreB Cells were sorted and transduced for 14 hours with ZsGr control virus (VC), A single virus listed (Only Vector), a viral mix of 37 different factors minus that listed virus (VM-Vector) or the viral cocktail of 37 factors (VM). Doxycycline was added for 24 hours and then cells were harvested, DNA isolated, and PCR analysis performed using the indicated primers.

FIG. 29 shows reverse cloning of reprogramming factors from myeloid (macrophage and granulocyte) colonies derived from reprogrammed pre/pro B cells. Examples of Gels run looking at 30 of the 37 different factors present in the cocktail. Notice that Evil, Msi2, Rux1t1, Hoxb3, and Pbx1 all have endogenous gene products present in every screen. White squares emphasize products that are at the correct size indicating integration of the factor listed.

FIG. 30 shows reverse cloning of reprogramming factors from myeloid (GEMM and B cell) colonies derived from reprogrammed pre/pro B cells. Examples of Gels run looking at 30 of the 37 different factors present in the cocktail. Notice that Evil, Msi2, Rux1t1, Hoxb3, and Pbx1 all have endogenous gene products present in every screen. White squares emphasize products that are at the correct size indicating integration of the factor listed.

FIG. 31 shows reverse cloning of reprogramming factors from myeloid (BFU) colonies derived from reprogrammed pre/pro B cells. Examples of Gels run looking at 30 of the 37 different factors present in the cocktail. Notice that Evil, Msi2, Rux1t1, Hoxb3, and Pbx1 all have endogenous gene products present in every screen. White squares emphasize products that are at the correct size indicating integration of the factor listed.

FIG. 32 shows frequency determination in which transcription factor combinations were reverse cloned in reprogrammed cells both intro (CFC colonies) and in vivo (donor-derived meyloid cells). To determine the individual factors contributing to the effects of the TF mix, integration primers were developed. ProPreB cells that gave rise to B cell (B cell), Macrophage (Mac), Granulocyte (Gran), Granulocyte-Macrophage (GM), Blast Forming Unit (BFU), GEMM, and those colonies not morphologically defined (Not Det) were collected and tested in the indicated n number. Similarly peripheral blood populations (B cell, macrophage, T cell, and other cells were tested for integration and grouped into the in vivo column. Results are summarized in a heat map. High prevalence in the population tested was visualized as red and low prevalence in the population was visualized as blue.

FIG. 33 shows reverse cloning of reprogramming factors from peripheral blood of mice reconstituted from ProPreB Cells expressing a combination of factors. Donor derived peripheral blood from the indicated mice (#4 and #5) reconstituted from ProPre B cells expressing a combination of factors was sorted and PCR analysis performed on the isolated DNA. Examples of two gels run looking at 30 of the 37 different factors present in the cocktail. Notice that Evil, Msi2, Rux1t1, Hoxb3, and Pbx1 all have endogenous gene products present in every screen. White squares emphasize products that are at the correct size indicating integration of the factor listed.

FIGS. 34A-34C demonstrate identity of factor combinations that are integrated into peripheral blood populations from a mouse reconstituted with ProPre B cells and CMPs transduced with the viral cocktail. For three of the transplanted mice (two originating from a transformed ProPre B cell and one from a CMP) that had peripheral chimerism >1.0% the peripheral blood was further sorted into B220+ (B cells), Mac+ (Mac) and CD3+ (T cells). FIG. 34A. Every peripheral bleed of donor derived cells originating from a reprogrammed ProPre B Cell or CMP contained Hlf, Zfp37, Runx1t1, Pbx1 and Lmo2. FIG. 34B. Additional factors identified in those populations are listed here. Notice that Prdm5 is present in all samples except those collect from the Mac1+ cells. Glis2 on the other hand was only found in Mac+ populations. FIG. 34C. Peripheral blood populations (B cell, macrophage, T cell, and other cells were tested for integration and grouped into the in vivo column for the n number of samples. Results are summarized in a heat map. High prevalence in the population tested was visualized as red and low prevalence in the population was visualized as blue.

FIG. 35 shows transcription factor combination lists. Six combinations (C1-C6) of 4-6 factors were put together based on the integration testing (>75% prevalence). To each combination the additional factors that were 50%-75% prevalent in the samples were added as additional factors (++). Each combination was derived from a specific colony or population. C1: ProPreB to Mac/Gran/GM; C2: ProPreB to GEMM/BFU, C3: ProPreB to BCell; C4: CMP toGEMM; C5: Overall In vitro; C6: Overall In vivo.

FIGS. 36A-36B show combinatorial expression of factors in ProPre B Cells increases colony formation. ProPre B Cells and CMPs were sorted using phenotypic markers on the Aria Sorter. Cells were incubated with ZsGr control virus (VC) or a viral cocktail for 14 hours in S-clone media containing SCF, TPO and IL-12 (In the case of ProPreB Cells, IL-7 and Flk3). Dox was added for 24 hours and cells were resorted for ZsGr+ cells. ZsGr+ cells were placed into methylcellulose media in a 6 well plate format containing SCF, TPO and IL-12 (For ProPreB Cells IL-7 and Flk3). Colony forming potential was assayed on day 20. FIG. 36A. To ensure that all factors in the combinations were required; factors were singly subtracted out of the combination. Representative pictures of the wells are shown. FIG. 36B. Quantitation of the data is demonstrated here. The ZsGreen control (VC) and the all the combination groups were performed in duplicates four independent experiments.

FIGS. 37A-37B demonstrate defined combinations of transcription factors can reprogram cells to different fates. ProPre B Cells and CMPs were sorted using phenotypic markers on the Aria Sorter. Cells were incubated with ZsGr control virus (VC) or a viral cocktail for 14 hours in S-clone media containing SCF, TPO and IL-12 (In the case of ProPreB Cells, IL-7 and Flk3). Dox was added for 24 hours and cells were resorted for ZsGr+ cells. ZsGr+ cells were placed into methylcellulose media in a 6 well plate format containing SCF, TPO and IL-12 (For ProPreB Cells IL-7 and Flk3). Colony forming potential was assayed on day 20. FIG. 37A. The morphology of each of the combinations is shown here. This again is an average of duplicate samples in four independent experiments. FIG. 37B. Representative pictures of transduced ProPreB cell CFC wells for combinations and controls are shown with composition break downs in pie charts for each combination (average of four experiments). Notice that C1 a myeloid promoting combination gave rise to predominantly myeloid cells. Which a B Cell promoting combination (C3) promoted predominantly B cell colonies.

FIG. 38 shows factor combination minus one experiments to determine the requirement of individual factors for reprogramming ProPre B Cells and CMPs were sorted using phenotypic markers on the Aria Sorter. Cells were incubated with ZsGr control virus (VC) or a viral cocktail for 14 hours in S-clone media containing SCF, TPO and IL-12 (In the case of ProPreB Cells, IL-7 and Flk3). Dox was added for 24 hours and cells were resorted for ZsGr+ cells. ZsGr+ cells were placed into methylcellulose media in a 6 well plate format containing SCF, TPO and IL-12 (For ProPreB Cells IL-7 and Flk3). Colony forming potential was assayed on day 20. To ensure that all factors in the combinations were required; factors were singly subtracted out of the combination. For each combination listed in bold the factors were subtracted out singularly. As a control Pbx1 (a factor not in the required combination was included as a control, as expected this additional factor was not a required factor in C2). Consistently all other combinations appeared to have been narrowed down to only required factors. Singular factor controls are listed in the last Figure. Bars represent averages of double samples performed in duplicate experiments.

FIG. 39 demonstrates that a defined set of factors identified to give rise to in vivo reprogramming and GEMM formation in myeloid colony forming assays can increase colony formation and alter the lineage potential of both ProPre B cells and CMPs.ProPre B Cells and CMPs were sorted using phenotypic markers on the Aria Sorter. Cells were incubated with ZsGr control virus (VC) or the defined combination C7 (C7) for 14 hours in S-clone media containing SCF, TPO and IL-12 (In the case of ProPreB Cells, IL-7 and Flk3). Dox was added for 24 hours and cells were resorted for ZsGr+ cells. ZsGr+ cells were placed into methylcellulose media in a 6 well plate format containing SCF, TPO and IL-12 (For ProPreB Cells IL-7 and Flk3). Colony forming potential was assayed on day 20.

FIGS. 40A-40B demonstrate that combination 6 leads to reprogramming of Pre-ProB cells into cells capable of giving rise to multi-lineage donor derived chimerism in vivo. ProPreB Cells and CMPs were sorted from CD45.2 rtTA transgenic bone marrow. Cells were then incubated with the indicated combination of factor expression viruses in equal concentrations. 10,000 Cells were then transplanted into congenic CD45.1+ mice. Mice were then bleed at 4, 8, 12, and 16 weeks. Only Combination 6 showed donor derived chimerism >1.0% in preliminary trials.

FIGS. 41A-41C demonstrate donor derived multi-lineage reconstitution from ProPre B Cells expressing a defined set of factors. ProPreB cells were transduced to express C6, C6 and the additional factors identified, ZsGr Control (VC). Cells were transplanted competitively into mice and peripheral bleeds performed at 4, 8 and 12 weeks. FIG. 41A. The gating strategy of mice transplanted with ProPre B Cells transduced with C6 and bleed at 4, 8, and 12 weeks. Donor-derived cells are observed over control level each bleed and are multi-lineage. FIG. 41B. Quantitations for all the bleeds for ProPreB cells are demonstrated. No benefit of the additional factors was observed. FIG. 41C. Cellular composition of the 12 week bleeds are shown in the graphs for ProPreB cells.

FIG. 42 demonstrates multi-lineage potential of reprogrammed B Cell progenitors by a defined set of factors (C6) is confirmed to have undergone recombination events and derived from B Cell origins. ProPreB cells were transduced to express C6, C6 and the additional factors identified, ZsGr Control (VC). Cells were transplanted competitively into mice and to demonstrate that the reconstitution was due to a cell that originated from a B cell, PCR analysis was performed on peripheral blood from the mouse that had long-term reconstitution in the peripheral blood. CD45.2+ donor Mac1+ cells had evidence of recombination events but recipient (CD45.1+) Mac1+ cells nor Fraction A B cells (B Cell Prog) had evidence of reprogramming.

FIG. 43 demonstrates a defined set of factors (C6) is expressed in peripheral blood derived from a reprogrammed ProPre B Cell. ProPreB cells were transduced to express C6, C6 and the additional factors identified, ZsGr Control (VC). Cells were transplanted competitively into mice and peripheral bleeds performed at 16 weeks. All the factors that were present in the viral mix were found to have integrated into the donor derived peripheral blood.

FIGS. 44A-44C demonstrate donor derived multi-lineage reconstitution from CMPs expressing a defined set of factors. FIG. 44A. CMP cells were transduced to express C6, C6 and the additional factors identified, ZsGr Control (VC). Cells were transplanted competitively into mice and peripheral bleeds performed at 4, 8 and 12 weeks. Lineage break down is shown by flow diagrams below for each mouse. FIG. 44B. Quantitation for all the bleeds for both CMPs derived reconstituting mice are demonstrated. No benefit of the additional factors was observed. FIG. 44C. Cellular composition of the 12 week bleeds are shown in the graphs for ProPreB cells.

FIG. 45 shows that reverse cloning confirms that donor derived peripheral blood originating from reprogrammed CMPs by C6 contains factors in Combination 6. CMP cells were transduced to express C6, C6 and the additional factors identified, ZsGr Control (VC). Cells were transplanted competitively into mice and a peripheral bleeds performed at 12 weeks. Peripheral blood was taken from both CMP originating iHSC reconstituting mice was taken and integration studies performed on the population. One mouse contained all factors used in the viral mix and the other was only missing Hlf.

FIGS. 46A-46C demonstrate a defined set of factors give rise to multi-lineage reconstitution from reprogrammed B Cells. Five additional factors were added to C6 that gave rise to GEMM colonies from either ProPre B cells or CMPs. This combination was coined C7. B220 enriched cells were magnetically separated from the bone marrow of CD45.2 rtTA mice. Cells were transduced with ZsGr control (VC) or C7 for 14 hours, kept for 24 hours with doxycycline and then transplanted competitively with 1×10̂5 whole bone marrow cells into CD45.1+ recipients. Bleeds were performed at 4, 8, 12, and 16 weeks. FIG. 46A. Flow plots are shown for both VC and C7 transduced and transplanted recipients at 8 weeks. FIG. 46B. Quantitation of peripheral bleeds for the B220 enriched cells transduced with ZsGr control (VC) or C7 at 4, 8, 12 and 16 weeks. Excluding one outlier all C7 transduced and transplanted mice are over VC transduced and transplanted cells. FIG. 46C. The average composition of peripheral blood at 4, 8, 12, and 16 weeks.

FIG. 47 shows multi-lineage reconstitution by reprogrammed B220 enriched cells has evidence of B cell recombination in 2/5 mice. Five additional factors were added to C6 that gave rise to GEMM colonies from either ProPre B cells or CMPs. This combination was coined C7. B220 enriched cells were magnetically separated from the bone marrow of CD45.2 rtTA mice. Cells were transduced with ZsGr control (VC) or C7 for 14 hours, kept for 24 hours with doxycycline and then transplanted competitively with 1×10̂5 whole bone marrow cells into CD45.1+ recipients. Bleed was performed at 16 weeks. To determine what reconstituted animals were derived from a B cell origin, peripheral blood was isolated, Mac1+ cells sorted, and tested by per analysis for B cell recombination. Two mice were found to have peripheral chimerism due to a transformed B cell. Those mice are shown in FIG. 40A by highlighting them in orange.

FIG. 48 shows that reverse cloning confirms that donor derived peripheral blood originating from reprogrammed CMPs by C7 contains factors in combination 7. Five additional factors were added to C6 that gave rise to GEMM colonies from either ProPre B cells or CMPs. This combination was coined C7. B220 enriched cells were magnetically separated from the bone marrow of CD45.2 rtTA mice. Cells were transduced with ZsGr control (VC) or C7 for 14 hours, kept for 24 hours with doxycycline and then transplanted competitively with 1×10̂5 whole bone marrow cells into CD45.1+ recipients. Bleed was performed at 16 weeks. Peripheral blood from the two B cell recombined mice was isolated and tested by per analysis for the integration of the factors in C7. Rbpms and Msi2 was missing from both analysis.

FIGS. 49A-49D show that peripheral lymphoid organ and bone marrow reconstitution is observed from CMPs and ProPreB Cells expressing a defined set of factors, combination 6. FIG. 49A. The bone marrow, spleen, and thymus were harvested from mice that were transplanted with C6 transduced ProPre B cells and CMPs. Quantitation of the data is graphically summarized. In all ProPreB cells transplanted mice with >1.0% peripheral blood chimerism, donor derived chimerism above control levels were observed in all lymphoid compartments analyzed. FIGS. 49B-49D. Composition of the bone marrow, spleen, and thymus for all control mice or experimental mice analyzed with >1% peripheral blood chimerism.

FIG. 50 demonstrates bone marrow reconstitution of the hematopoietic progenitor and myeloid progenitor compartments is observed when CMPs and ProPreB Cells expressing a defined set of factors, combination 6, are transplanted. The bone marrow was harvested from mice transplanted with ProPreB Cells/CMPs transduced with control (VC) a defined viral cocktail (C6). Representative histograms are shown of populations reprogrammed with C6: two CMP transplanted mice (CMP1 and CMP2) and one ProPre B Cell transplanted mouse (ProPreB1). Cells have been previously gated for singlets, live, lineage negative cells. Varying degrees of donor derived chimerism can be observed. The c-kit and sca graphs show that there is donor derived hematopoietic progenitors (LSK; c-kit+Sca+) and myeloid progenitors (Myl Pro; c-kit+Sca−).

FIGS. 51A-51C demonstrate that ProPreB Cells and CMPs expressing a defined set of factors (C6) give rise to primitive hematopoietic progenitors. The bone marrow was harvested from mice transplanted with ProPreB Cells/CMPs transduced with control (VC) a defined viral cocktail (C6). Representative histograms are shown of populations reprogrammed with C6: two CMP transplanted mice (CMP1 and CMP2) and one ProPre B Cell transplanted mouse (ProPreB1). Graphs represent donor (CD45.2+) derived hematopoietic progenitors (LSK; c-kit+Sca+) and myeloid progenitors (Myl Pro; c-kit+Sca−). FIG. 51A. Quantitation of the overall numbers of myeloid progenitors and hematopoietic progenitor cells in each of the transplanted VC (average of five mice) and C6 mice with peripheral chimerism above 1.0%. In all cases there is increased numbers of cells with respect to controls. FIGS. 51B-51C. Composition of the compartments was analyzed and quantified. Each bar represents one mouse and the respective composition of the myeloid progenitor compartment (FIG. 51B) or the hematopoietic progenitor compartment (FIG. 51C).

FIG. 52 demonstrates that reprogrammed CMPs by defined factors have serial transplantation potential. 16 weeks bone marrow analysis was performed and secondary transplants set up. The two CMP derived mice with donor derived chimerism underwent full bone marrow transplant of 5 million donor cells into five mice each. In the case of the mouse having donor derived chimerism originating from a ProPre B cell transduced with C6, 1 million whole donor bone marrow cells were competitively transplanted with 2×10̂5 CD45.1+ whole bone marrow cells into two mice. Flow graphs of donor derived cells from each of these mice are shown. Donor cells are observed at 4 weeks.

FIGS. 53A-53C demonstrate that reprogrammed CMPs by defined factors have serial long-term transplantation potential. 16 weeks bone marrow analysis was performed and secondary transplants set up. The two CMP derived mice with donor derived chimerism underwent full bone marrow transplant of 5 million donor cells into five mice each. In the case of the mouse having donor derived chimerism originating from a ProPre B cell transduced with C6, 1 million whole donor bone marrow cells were competitively transplanted with 2×10̂5 CD45.1+ whole bone marrow cells into two mice. Flow graphs of donor derived cells from each of these mice are shown. Donor cells are observed at 4 weeks. FIG. 53A. An example of multilineage donor chimerism at 4 weeks in the peripheral blood of secondary transplants. FIG. 53B. Quantitation of CD45.2+ donor contributions in peripheral blood at 4 and 8 weeks. CMPs transduced with C6 gave rise to multilineage chimerism in primary recipients and in secondary transplants all the mice had donor cells. FIG. 53C. Quantitation of the composition of peripheral blood cells in secondary recipients.

FIG. 54 demonstrates that peripheral blood derived from CMP C6 reconstituted mice can be reprogrammed to give rise to in vitro colony forming potential. Peripheral blood from serially transplanted C6 transduced CMP cells was collected. B220+ and CD3+ and Mac1+ cells were sorted and incubated for 48 hours with doxycycline. Cells were then put into methylcellulose media containing SCF, TPO, IL-12, Flk3, and IL-7. Colonies in the CFCs assays were counted and morphology characterized 20 days later. Control sorted cells from primary VC recipients were blank but colonies were observed when cells were derived from CMPs previously transduced with C6.

FIG. 55 demonstrates that peripheral blood derived from reconstituted mice having been transplanted with B220 enriched cells expressing C7 mice can undergo secondary reprogrammed to give rise to in vitro colony forming potential. Peripheral blood from mice transplanted with B220 enriched cells expressing combination C7 was collected at 16 weeks. B220+ and CD3+ and Mac1+ cells were sorted and incubated for 48 hours with doxycycline. Cells were then put into methylcellulose media containing SCF, TPO, IL-12, Flk3, and IL-7. Colonies in the CFCs assays were counted and morphology characterized 20 days later. Control sorted cells from primary VC recipients were blank but colonies were observed when cells were derived from the peripheral blood of either mouse reconstituted from reprogrammed B220 enriched cells expressing C7.

FIGS. 56A-56C demonstrate that expression of defined factors in various populations can promote colony formation and altered lineage commitment in vitro. Various indicated populations were sorted from the bone marrow (FIG. 56A), spleen (FIG. 56B), thymus (FIG. 56C), and peripheral blood (FIG. 56C) of mice. Populations include: B220+ (B); Mac1+/Gr-1+ (M/G); CD3+/CD4+/CD8+ (T); NK1.1+ (NK); ProPreBCells as a control. In the case of peripheral blood (PB) B, T, and M/G was all sorted into one population. These populations were transduced with control (VC) or C7 viruses for 14 hours, dox added for 24 hours and then put into a CFC assay. Colonies were counted and morphology determined on day 20. Colony numbers with more than control levels in almost all cases. Indicating that transformation of committed blood cells into iHSC like cells could occur from multiple compartments and in multiple cell types.

FIGS. 57A-57C demonstrate that expression of defined factors in human Jurkat cells can promote colony formation and altered lineage commitment in vitro. FIG. 57A. Human Jurkat cells were cultured and left untransduced, transduced with ZsGr control virus (VC) or with C6 for 14 hours. Doxycycline was added for 24 hours and cells were put in CFC assays. Colonies were counted and morphology determined on day 20. Only Jurkat cells transduced with C6 gave rise to colonies. FIG. 57B. Colonies that Jurkat cells transduced with C6 gave rise too are pictured. They included an erythroid like colony, granulocytes, and monocytes. FIG. 57C. To further distinguish the transformed cells, flow analysis for phenotypic markers including Ter119, Mac1, CD71, and Gr1 was performed on freshly cultured Jurkat cells and the Jurkat cell colonies observed when transduced with C6. Jurkat colonies that were transduced with C6 had apparent increases in immature erythroid cells (CD71+Ter119−), Granulocyte (Gr1+Mac1+) and monocyte (Mac1+) populations.

FIGS. 58A-58E show identification of factors capable of imparting alternative lineage potential in vitro. (FIG. 58A) Heat map showing relative expression (green;high, to purple;low) of 36 regulatory genes identified as HSC-specific in the indicated cell types. (FIG. 58B) Schematic representation of lentivirus transgene expression cassette (top), and flow cytometry plots showing reporter cassette (ZsGr) expression in Pro/Pre B-cells+/− doxycycline induction (48 hours post). (FIG. 58C) Schematic representation of in vitro screening strategy for cell fate conversion. (FIG. 58D) Representative images of wells showing colonies arising in methylcellulose from Pro/Pre B cells transduced with ZsGr or 36-factor cocktail. (FIG. 58E) Colony number and type arising in methylcellulose from Pro/Pre B cells transduced with ZsGr or 36-factor cocktail. Four independent experiments are shown and each condition performed in triplicate.

FIGS. 59A-59G show identification of factors capable of imparting multi-lineage engraftment potential onto committed progenitors in vivo. (FIG. 59A) Schematic of experimental strategy to identify factors capable of imparting multi-lineage engraftment potential on committed progenitors in vivo. (FIG. 59B) Representative flow cytometry plots showing donor (CD45.2) reconstitution of mice transplanted with control (ZsGr) or 36-factor transduced Pro/Pre B cells or CMPs 16-weeks post-transplant. (FIG. 59C) Donor reconstitution of mice transplanted with ZsGr or 36-factor transduced Pro/Pre B cells or CMPs at indicated time points post-transplantation. Only mice with >1% donor chimerism (dotted line) were considered reconstituted. Recipients transplanted; Pro/PreB; ZsGr n=15, Pro/PreB; 36-factor n=15, CMP; ZsGr n=8, and CMP; 36-factor n=8. (FIG. 59D) Reconstitution of indicated peripheral blood cell lineages of individual recipients showing >1% donor chimerism presented as % of donor. (FIG. 59E) PCR analysis of immunoglobulin rearrangement showing heavy (J_{H}), and light chain (J_{Lλ}, J_{LK}) in bone marrow (BM) cells including B-cells (B220+), stem/progenitor (LSK) cells, myeloid progenitors (Myl Pro), and peripheral blood (PB) cells including B-cells (B220+), recipient myeloid cells (Mac1+ Rec), and donor myeloid cells (Mac1+ Donor) originating from Pro/Pre B cell; 36-factor experiment. Loading control; genomic PCR for CD45. (FIG. 59F) PCR-based strategy to identify virally integrated factors and discriminate from endogenous genes. (FIG. 59G) Summary of data showing presence (gray) or absence (black) of each of the indicated factors in donor B−, T−, and myeloid cells in each of the reconstituted mice shown in (FIG. 59C).

FIGS. 60A-60G show transient ectopic expression of six transcription factors in committed progenitors is sufficient to alter lineage potential in vitro and impart long-term engraftment potential on committed progenitors in vivo. (FIG. 60A) Representative images of wells showing colonies arising in methylcellulose from Pro/Pre B cells transduced with ZsGr or 6-TF cocktail. (FIG. 60B) Colony number and indicated colony type arising in methylcellulose from Pro/Pre B cells transduced with ZsGr or 6-TF cocktail. 3 independent experiments are shown with each condition performed in triplicate. (FIG. 60C) Colony number and type arising in methylcellulose from Pro/Pre B cells transduced with ZsGr, 6-TF cocktail, or 6-TF minus the indicated factor. Each condition performed in triplicate. (FIG. 60D) Donor reconstitution of mice transplanted with ZsGr or 6-TF transduced Pro/Pre B cells or CMPs at indicated time points post-transplantation. Only mice with >1% donor chimerism (dotted line) were considered reconstituted. Recipients transplanted; Pro/PreB; ZsGr n=10, Pro/PreB; 6-TF n=12, CMP; ZsGr n=9, and CMP; 6-TF n=9. (FIG. 60E) Representative flow cytometry plots showing donor reconstitution and lineage composition of mice transplanted with control (ZsGr) or 6-TF transduced Pro/Pre B cells or CMPs 16-weeks post-transplant. Lineage contribution to Mac1+ myeloid cells, B220+ B-cells, and CD3/4/8+ T-cells is shown. (FIG. 60F) Reconstitution of indicated peripheral blood cell lineages of individual recipients showing >1% donor chimerism presented as % of donor. (FIG. 60G) PCR analysis of immunoglobulin heavy (JH) chain rearrangement in recipient myeloid cells (Mac1+ Rec), and donor myeloid cells (Mac1+ Donor) originating from Pro/Pre B cell; 6-TF experiment. Loading control; genomic PCR for CD45.

FIGS. 61A-61E show inclusion of Meis1 and Mycn and use of polycistronic viruses improves in vivo reprogramming efficiency. (FIG. 61A) Schematic representation of RHL (Runxt1t1, Hlf, Lmo2) and PZP (Pbx1, Zfp37, Prdm5) polycistronic, and Meis1 and Mycn single factor viral constructs. (FIG. 61B) Donor reconstitution of mice transplanted with ZsGr, 8-TF (8 single factor viruses), or 8-TFPoly (RHL, PZP polycistronic viruses plus Meis1 and Mycn viruses), transduced Pro/Pre B cells at indicated time points post-transplantation. Only mice with >1% donor chimerism were considered reconstituted. Recipients transplanted; ZsGr; n=12, 8-TF; n=6, 8TFPoly; n=14. (FIG. 61C) Representative flow cytometry plots showing donor reconstitution and lineage contribution of mice transplanted with control (ZsGr), 8-TF, or 8TFPoly transduced Pro/Pre B cells 16-weeks post-transplant. Lineage contribution to Mac1+GR1− myeloid cells, Mac+GR1+ granulocytes, B220+ B-cells, and CD3/4/8+ T-cells is shown. (FIG. 61D) Reconstitution of indicated peripheral blood cell lineages of individual recipients showing >1% donor chimerism presented as % of donor. (FIG. 61E) PCR analysis of immunoglobulin heavy (JH) chain rearrangement in recipient (Recip), and donor (Donor) myeloid cells. Loading control; genomic PCR for CD45.

FIGS. 62A-62I shows reprogrammed cells engraft secondary hematopoietic organs, bone marrow progenitor compartments and reconstitute secondary recipients. (FIG. 62A) Donor reconstitution of peripheral blood (PB), bone marrow (BM), spleen, and thymus of mice transplanted with 8-TF, or 8-TFPoly transduced Pro/Pre B cells 18-20 weeks post-transplantation. (FIG. 62B) PCR analysis of immunoglobulin heavy (J_{H}) chain rearrangement in recipient (R), and donor (D) cells. Cell types analyzed include Mac1+ myeloid cells (M), Mac1+GR1+ granulocytes (G), and T-cells (T). Loading control; genomic PCR for CD45. (FIG. 62C) Representative bone marrow stem and progenitor analysis of a recipient transplanted with 8-TFPoly transduced Pro/Pre B cells 18-weeks post-transplantation showing donor-reconstitution of myeloid progenitors (Myl Pro), megarkaryocyte/erythrocyte progenitors (MEP), granulocyte/monocyte progenitors (GMP), common myeloid progenitors (CMP), megakaryocyte progenitors (MkP), erythroid progenitors (EP), common lymphoid progenitors (CLP), Lineage-negative Sca1+ckit+ multipotent progenitors (LSK), multipotent progenitors (MPP1, MPP2), and hematopoietic stem cells (HSC). All cells were pre-gated through doublet-discriminated, live (propidium iodide negative), and lineage negative cells. (FIG. 62D) Total donor reconstitution of the indicated populations in mice analyzed in (FIG. 62A). (FIGS. 62E-62F) Reconstitution of the indicated myeloid progenitor (E) and primitive multi-potent and stem cell (F) populations in mice analyzed in (A) presented as percentage of donor. (FIG. 62G) PCR analysis of immunoglobulin heavy (JH) chain rearrangement in the indicated recipient and donor populations. Loading control; genomic PCR for CD45. (FIG. 62H) Donor reconstitution of secondary recipient mice transplanted with whole bone marrow (WBM) or c-Kit positive bone marrow cells derived from primary transplants of 8-TF transduced Pro/Pre B cells analyzed at 12 and 8 weeks respectively. Number of recipients transplanted; WBM; n=5, c-Kit+; n=4. (FIG. 62I) Reconstitution of indicated peripheral blood cell lineages of individual recipients presented as % of donor.

FIGS. 63A-63H show transient expression of defined transcription factors in myeloid effector cells is sufficient instill them with progenitor activity in vitro, and long-term multi-lineage transplantation potential in vivo. (FIG. 63A) Schematic representation of experimental strategy for assaying the colony forming potential of 8-TF transduced peripheral blood cells. (FIG. 63B) Colony number and type arising in methylcellulose from peripheral blood cells from recipient (left-most lanes) or donor cells derived from a recipient transplanted with Pro/Pre B cells transduced with 8-TF or 8-TFPoly cocktail, plus (+) or minus (−) exposure to doxycycline. Results from individual mouse performed in triplicate are shown. (FIG. 63C) Colony number and type arising in methylcellulose from plated granulocytes, macrophages/monocytes (Myl), B-cells, and T-cells purified from the peripheral blood of cells pooled recipients transplanted with Pro/Pre B cells transduced with 8-TF^{Poly} cocktail plus (+) or minus (−) exposure to doxycycline. (FIG. 63D) Representative colony types and cytospins stained with May Grunwald of colonies derived in (FIG. 63C). (FIG. 63E) Donor reconstitution of mice transplanted with ZsGr, 6-TF^{Poly}, 8-TF or 8-TF^{Poly} transduced Mac1+cKit− myeloid effector cells at indicated time points post-transplantation. Only mice with >1% donor chimerism were considered reconstituted. Recipients transplanted; ZsGr; n=6, 6-TF^{Poly}; n=7, 8-TF; n=6, and 8-TFPoly; n=8. (FIG. 63F) Reconstitution of indicated peripheral blood cell lineages of mice showing >1% donor chimerism presented as % of donor. (FIG. 63G) Donor reconstitution 12 weeks post-transplant of secondary recipient mice transplanted non-competitively with 5×10⁶ donor-derived (CD45.2+) bone marrow cells derived from primary recipients of 6-TF^{Poly}, 8-TF or 8-TF^{Poly} transduced Mac1+cKit− myeloid effector cells. Cells from individual primary donor mice (indicated by ID) were transplanted into N=5 secondary recipients each. (FIG. 63H) Average reconstitution of indicated peripheral blood cell lineages presented as % of donor. N=5 recipients per group.

FIGS. 64A-64D shows iHSCs reprogrammed via 8 transcription factors closely resemble endogenous HSCs at the molecular level. FIG. 64A shows phenotypic HSCs (doublet discriminated, live, lineage negative, c-kit+, Sca1+, CD34−, flk2− and CD150+) were FACS sorted from the bone marrow of mice reconstituted with Pro/Pre B cells transduced with 8-TF (Mouse #1) and 8-TF POLY (Mouse #10) viral cocktails. Cells were single cell sorted into 96 well plates and analyzed by qPCR for an array of transcription factors. Expression levels of individual cells were projected onto a three-dimensional space using principle component analysis. Recipient HSCs (HSC Host) and iHSCs derived from Pro/Pre B cells transduced with 8-TF (iHSC 8-TF) or 8-TF Poly (iHSC 8-TF Poly) were displayed with previously profiled and phenotypically characterized progenitor cells: HSC, MPP, CMP, GMP, MEP and CLP. Additionally, Pro/Pre B Cells were added as a control cell type. FIGS. 64B-C shows phenotypic HSCs isolated from bone marrow reconstituted from Pro/Pre B cells transduced with 8-TF (iHSC 8-TF) and 8-TF^{Poly} (iHSC 8-TF^{Poly}) were then hierarchically clustered with respect to the qPCR transcription factor array. Each leaf of the dendrogram represents a single cell as indicated in the legend in panel A. FIG. 64D shows analysis of indicated genes are shown for: phenotypic control HSCs (HSC), transplanted host HSCs (HSC host), iHSCs derived from Pro/Pre B Cells transduced with 8-TF (iHSC 8-TF) and 8-TF POLY (iHSC 8-TFPoly) and control Pro/Pre B Cells. Heat maps for expression levels in the indicated cell types are shown (high expression was visualized as red; low expression was visualized as blue). Violin plots show distribution patterns of each of the above transcription factors in one cell type. Expression level is on the y-axis.

FIGS. 65A-65B show a sorting strategy for Pro/Pre B cells (FIG. 65A) and CMPs (FIG. 65B) from the bone marrow of rtTA transgenic mice. Doublet discriminated and PI negative cells were pre-gated and Pro/Pre B Cells were gated as indicated: B220+ CD19+, AA4.1+ and IgM−. FIG. 65B shows doublet discriminated and PI negative cells were pre-gated and CMPs were gated as indicated: Lineage negative (Gr1−, Mac1−, B220−, CD3−, CD4−, CD8−, Ter119−), c-kit+, Sca1−, Fc□R3MID, and CD34+.

FIG. 66 shows Pro/Pre B cells and CMPs were transduced with the viral cocktail of 36-TFs. Dox is added after 16 hours for a period of 48 hours before cells were transferred to methylcellulose. 20 days later colonies were counted and characterized by morphology as indicated in FIGS. 59A-59G. Colonies were collected and DNA isolated. Identification of plasmid integration was performed as indicated in FIGS. 60A-60G for each of the 36 factors listed. Expression of the factors was clustered by the highest expression in GEMMs.

FIG. 67 shows Mac1+ bone marrow cells were isolated from transgenic rtTA mice. Cells were transduced for 16 hours with RHL+PZP (6-TF POLY), Runx1t1+Hlf+Lmo2+Pbx1+Zfp37+Prdm5+Mycn+Meis1 (8-TF) and RHL+PZP+Mycn+Meis1 (8-TF POLY). Dox was added in culture for 24 hours and 5.0×10⁶ cells were transplanted into conditioned hosts with 1×10⁵ Scat depleted support cells. Peripheral blood analysis was performed at 6 weeks. Representative flow demonstrating CD45.1+ (donor) gating from peripheral bleeds at 16 weeks is shown for each group.

FIGS. 68A-68D show Mac1+ bone marrow cells were FACS sorted, transduced with ZsGr control, 6-TF, 8-TF, or 8-TF POLY viruses. (FIG. 68A) Transplantation was done as indicated and 18 weeks post transplantation bone marrow, spleen, thymus, and peripheral blood was harvested from mice with peripheral blood reconstitution >5.0%. Donor contributions are shown graphically in the peripheral blood (PB), bone marrow (BM), spleen and thymus for a 6-TF POLY mouse, 8-TF mouse and four 8-TF POLY mice. The y-axis break marks 1.0% donor reconstitution. FIG. 68B shows the composition break down for donor-derived cells in the bone marrow, spleen, and thymus. B cells (B), Granulocytes (G), Myeloid (M) and T Cells (T) were phenotypically defined as previously described. FIG. 68C shows the % donor of each of the progenitor compartments was calculated by gating as previously shown but last through donor. Quantitation of these results is shown for mice reconstituted from Mac1+ bone marrow cells transduced with 6-TF POLY (1 mouse), 8-TF (1 mouse) and 8-TF POLY (4 mice). A break indicates a 1.0% donor composition. FIG. 68D shows compositional breakdown of the Hematopoietic progenitor compartment for each mouse reconstituted from Mac1+ bone marrow cells transduced with 6-TF POLY (1 mouse), 8-TF (1 mouse) and 8-TF POLY (4 mice). Populations were gated first by donor and then by previously defined phenotypic markers.

FIG. 69 shows phenotypic HSCs (doublet discriminated, live, lineage negative, c-kit+, Sca1+, CD34−, flk2− and CD150+) were FACS sorted from the bone marrow of mice reconstituted with Pro/Pre B cells transduced with 8-TF and 8-TF POLY viral cocktails. Cells were single cell sorted into 96 well plates and analyzed by qPCR for an array of transcription factors. A heat map displays transcription factor expression (columns) for indicated cell types (rows), including: previously profiled and phenotypically sorted progenitor control cell types (HSC, MPP, MEP, CMP, GMP, CLP), control Pro/Pre B cells, recipient derived HSCs (Host HSC), and iHSC cells isolated from mice reconstituted from Pro/Pre B Cells transduced with viral mixtures of 8-TF (iHSC 8-TF) and 8-TF POLY (iHSC 8-TF POLY). High expression was visualized as red; Low Expression was visualized as blue.

FIGS. 70A-70H shows reprogramming terminally differentiated myeloid cells to engraftable HSC-like cells. (FIG. 70A) Schematic for secondary reprogramming experiments. Peripheral blood post 16 weeks from mice reconstituted from ProPre B Cells transduced with viral mixes of 8-TFs were isolated. Peripheral blood cells, FACS sorted CD45.1+ (donor) or further purified on magnetic columns for B220+ (B Cells), Mac1+ (Myl), Gran (Mac1+ Gr1+) and T cells (CD3+). Cells were then plated into F12 media in the presence or absence of dox. Three days post dox administration, cells are transferred into methylcellulose. Colonies are counted and scored 20 days later. (FIG. 70B) Mice reconstituted with ProPre B Cells transduced with the viral cocktail 8-TF or 8-TF POLY were bled at 16-20 weeks and CD45.1+ (donor) and CD45.2+ (Recipient) cells were FACS sorted (8-TF) or unsorted (8-TF POLY), plated into F12 media in the presence/absence of dox for 3 days, transferred into methylcellulose, and counted/scored on day 20. Quantitation of the colony number and composition is shown for cells in the presence and absence of dox. Each column represents one or three replicates per mouse. A representative GEMM colony and GM (Granulocyte-Myeloid) colony are shown to the right for donor sorted cells in the presence of dox. (FIG. 70C) Mice reconstituted with ProPre B Cells transduced with 8-TF POLY were bled at 16 weeks and CD45.1+ (donor) and CD45.2+ (recipient) cells were pooled, further enriched using magnetic columns for B220+ (B Cells), Mac1+ (Myl), Mac1+ Gr1+ (Gran) and CD3+ (T Cells). Cell populations were plated into F12 media in the presence/absence of dox for 3 days, transferred to methylcellulose, and counted/scored on day 20. Quantitation of the colony number and composition is shown for cells in the presence and absence of dox. (FIG. 70D) Representative 10× views of colonies [GEMM, GM, Granulocyte (G) and Myeloid (M)] derived from donor cells are shown. Cytospins were performed on each colony and shown to the right with prominent cell types labeled. (FIG. 70E) Mac1+ bone marrow cells were isolated from transgenic rtTA mice. Cells were transduced for 16 hours with RHL+PZP (6-TF POLY), Runx1t1+Hlf+Lmo2+Pbx1+Zfp37+Prdm5+Mycn+Meis1 (8-TF) and RHL+PZP+Mycn+Meis1 (8-TF POLY). Dox was added in culture for 24 hours and 5.0×106 cells were transplanted into conditioned hosts with 1×10⁵ Sca1 depleted support cells. Peripheral blood analysis was performed at 4, 8, 12 and 16 weeks; donor contributions are summarized in the graph. Each circle represents a mouse and the 1% donor chimerism mark is represented by an axis break. (FIG. 70F) Composition of mice reconstituted over 1% are shown and broken into B cell, myeloid, granulocyte, and T cell as previously defined. (FIG. 70G) Secondary transplantation was performed by euthanizing and harvesting bone marrow from primary mice with donor reconstitutions over 5%. Five million FACS sorted donor (CD45.2+) whole bone marrow cells were transplanted non-competitively into five recipient pre-conditioned mice. Peripheral blood chimerism at 16 weeks is shown for each secondary recipient (each circle). (FIG. 70H) The average composition of the donor-derived cells in the secondary transplant was calculated and graphically represented for 16 week bleed data.

FIGS. 71A-71B show donor-derived bone marrow, originating from transformed Pro/Pre B-Cells, was isolated from two primary reconstituting animals and one secondary animal. B220+ (B-Cells), CD3+ (T-Cells), Mac1+Gr1− (Myeloid) and Mac1+Gr1+ (Gran) cells were FACS sorted. VDJ analysis was performed on each of the lineages, similar size bands were selected and individual VDJ amplicons were sequenced to obtain information on individual recombination events in each of the lineages. Sequence data is show for each of the indicated donors/cell types. Using IgBlast (http://www.ncbi.nlm.nih.gov/igblast/) VDJ recombinational events were identified (VDJ ID) and listed according to the VH, DH or JH segment to which the sequence corresponds. (FIG. 71A) Sequences for Donor 1°-1 are disclosed as SEQ ID NOS 168-169, 168-169, 176, 176, 176, 176, 181, 181, 181 and 181 read from columns left to right. Sequences for Donor 1°-8 are disclosed as SEQ ID NOS 170, 170, 170, 170, 177, 177, 177, 177, 182, 182, 182 and 182 read from columns left to right. (FIG. 71B) Sequences for Donor 2°-1 are disclosed as SEQ ID NOS 168, 168, 168, 171-175, 176, 176, 176, 178-180, 180, 183, 183, 183-185, 185-186 and 186 read from columns left to right.

FIGS. 72A-72C Donor-derived MEP cells (Live, Lin−, c-kit+, Sca1−, CD34−, FcgR3−) were FACS sorted from the bone marrow of a primary recipient reconstituted from a transformed Pro/Pre B-Cell (Mouse ID 6). MEP cells were transplanted into three irradiated recipients (50,000 cells/recipient). Controls were irradiated but not transplanted. (FIG. 72A) The survival of these mice is indicated graphically over time post transplant. At day 20 post transplant the peripheral blood of the remaining mice was tested for red blood cell counts (RBC Counts, FIG. 72B) and platelet numbers (Platelet Counts, FIG. 72C).

### DETAILED DESCRIPTION

Provided herein are compositions, nucleic acid constructs, methods and kits thereof for hematopoietic stem cell induction or reprogramming cells to the hematopoietic stem cell multipotent state, based, in part, on the discoveries described herein of novel combinations of transcription factors that permit dedifferentiation and reprogramming of more differentiated cells the hematopoietic stem cell state. Such compositions, nucleic acid constructs, methods and kits can be used for inducing hematopoietic stem cells in vitro, ex vivo, or in vivo, and these induced hematopoietic stem cell can be used in regenerative medicine applications.

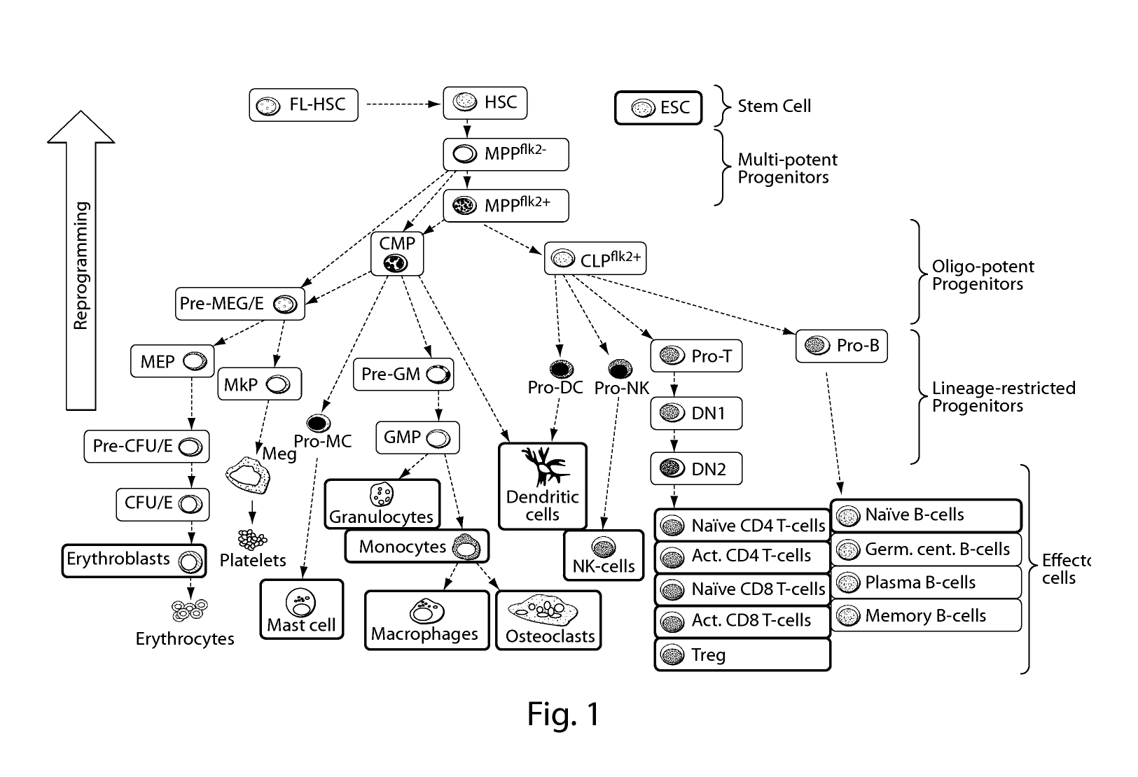

Hematopoietic stem cells (HSCs) are among the best-characterized and most experimentally tractable tissue-specific stem cells. HSCs reside at the top of hematopoietic hierarchy and give rise to a large repertoire of highly specialized effector cells by differentiating through a succession of increasingly committed downstream progenitor cells (FIG. 1). HSCs are the only cells in the hematopoietic system that possess the ability to both differentiate to all blood lineages and to self-renew for life. These properties, along with the ability of HSCs to engraft conditioned recipients upon intravenous transplantation, have established the clinical paradigm for stem cell use in regenerative medicine. Allogeneic and autologous HSC transplantation are routinely used in the treatment of patients with a variety of life-threatening disorders. Despite wide clinical use, HSC transplantation remains a high-risk procedure, with the number of stem cells available for transplantation being the strongest predictor of transplantation success. Although stem cell mobilization with G-CSF alone, or in combination with other drugs, increases the yield of hematopoietic stem cells for transplantation, an ability to induce, expand, or generate patient-specific HSCs de novo, as described herein, could be useful in a number of clinical settings, or be used to model hematopoietic diseases ex vivo or in xenotransplantation models.

The developmental process by which differentiated cell types arise from more primitive progenitor cells is guided in part by progressive epigenetic changes. In general, lineage specification is unidirectional and irreversible with differentiated cell types, and even intermediate progenitors, being remarkably fixed with respect to their cellular identity and developmental potential. Studies by Gurdon and others have demonstrated that the process of differentiation can be reversed in experiments that showed that the nuclei of differentiated cell types could be reprogrammed to totipotency when exposed to the primitive cellular milieu of enucleated oocytes. This process, known as “somatic cell nuclear transfer,” was subsequently shown to be capable of reprogramming nuclei from differentiated mammalian cells back to pluripotency. That ectopic expression of defined transcription factors was sufficient to convert cell fate was first shown in 1987 with the demonstration that enforced expression of MyoD could reprogram fibroblasts to the myogenic lineage. Enormous progress in this field has been made over the past 40 years culminating with the striking demonstration by Yamanaka and colleagues that ectopic expression of four transcription factors (c-Myc, Oct4, Klf4, Sox2, the so-called “Yamanaka factors”) also described in e.g., U.S. Pat. No. 7,964,401; U.S. Pat. No. 8,048,999; U.S. Pat. No. 8,058,065; U.S. Pat. No. 8,129,187; U.S. Pat. No. 8,211,697, can reprogram adult fibroblasts from mice and man into cells, termed iPS (induced pluripotent stem) cells, that possess the developmental potential of embryonic stem (ES) cells. These discoveries opened the possibility of generating patient-specific pluripotent cells from abundant somatic cells that could be used to model disease, or for autologous cell replacement therapies.

However, these factors do not replicate this process if the starting cell is a cell from hematopoietic lineage.

Despite their enormous promise, significant hurdles must be overcome before iPS-based cell therapies enter the clinic. It must also be recognized that iPS cells cannot be directly used clinically, since—as is the case with ES cells—useful cell types must first be generated by directed differentiation.

Thus, alternative approaches, in which abundant cell types are directly reprogrammed to alternative fates without first returning to a pluripotent state, as described herein for making induced HSCs, can be a more direct and efficient way to generate clinically useful cell types. For example, a recent report using OCT4 in combination with hematopoietic cytokines also showed that it was possible to generate myeloid lineage hematopoietic cells (though not HSCs) from human fibroblasts.

Differentiation of HSCs to fully differentiated blood cells is believed to be an irreversible process under normal physiological conditions. Hematopoietic lineage specification takes place within the bounds of strict lineal relationships: for example, megakaryocyte progenitors give rise to megakaryocytes and ultimately platelets, but not to any other blood lineages. Some studies, however, have demonstrated that hematopoietic cells are amenable to reprogramming to alternative fates under experimental manipulation.

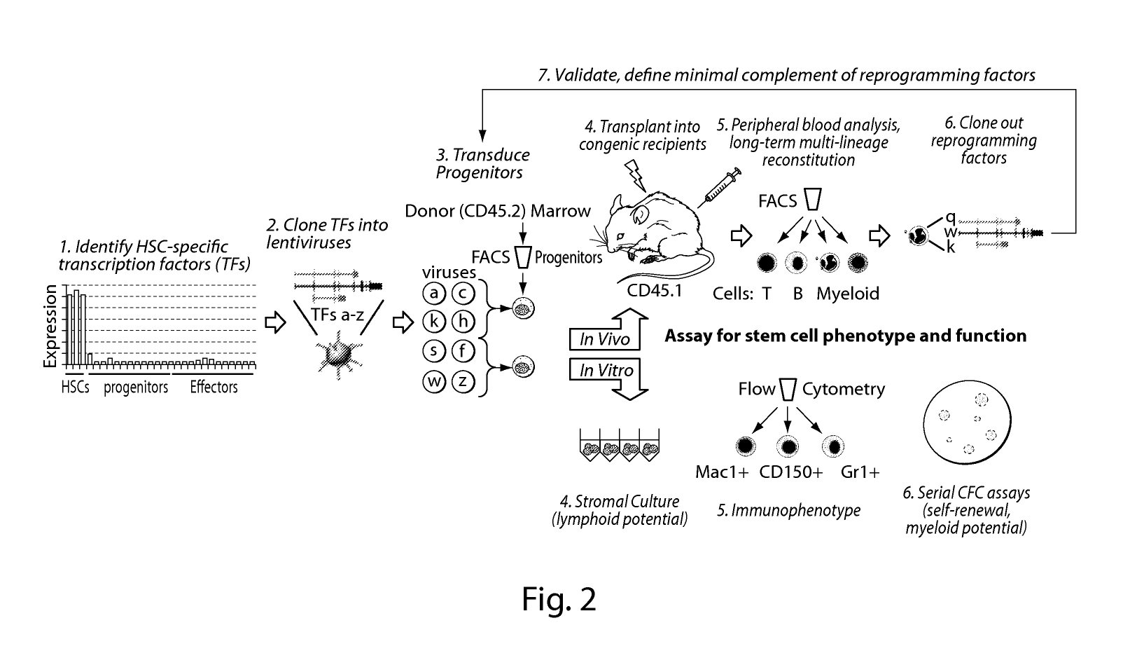

Within the hematopoietic system, the most clinically useful cell type to strive to generate by reprogramming are HSCs, as they are the only cells which possess the potential to generate all blood cell types over a lifetime, and transplantation protocols for their clinical use are already established. To date, no reports describing the generation of HSCs by reprogramming have been published because the factor(s) needed to reprogram to HSCs have not yet been determined. This point is central to the experimental rationale and strategies described herein, which were designed to first identify and clone transcriptional activators important for specifying HSC fate and function, and then utilize such factors to reprogram committed blood cells back to an induced HSC fate (FIG. 2), as demonstrated herein.

Hematopoietic tissues contain cells with long-term and short-term regeneration capacities, and committed multipotent, oligopotent, and unipotent progenitors. Endogenous HSCs can be can be found in a variety of tissue sources, such as the bone marrow of adults, which includes femurs, hip, ribs, sternum, and other bones, as well as umbilical cord blood and placenta, and mobilized peripheral blood. Endogenous HSCs can be obtained directly by removal from, for example, the hip, using a needle and syringe, or from the blood following pre-treatment with cytokines, such as G-CSF (granulocyte colony-stimulating factors), that induce cells to be released from the bone marrow compartment. However, such methods yield varying amounts of HSCs, which are oftentimes not enough for use in treatment options.

Accordingly, “hematopoietic stem cells,” or “HSCs,” as the terms are used herein, encompass all multipotent cells capable of differentiating into all the blood or immune cell types of the hematopoietic system, including, but not limited to, myeloid cells (monocytes and macrophages, neutrophils, basophils, eosinophils, erythrocytes, megakaryocytes/platelets, dendritic cells), and lymphoid lineages (T-cells, B-cells, NKT-cells, NK-cells), and which have multi-lineage hematopoietic differentiation potential and sustained self-renewal activity.

The term “stem cells,” as used herein, refer to cells that retain the ability to renew themselves through mitotic cell division and can differentiate into a diverse range of specialized cell types. The two broad types of mammalian stem cells are: embryonic stem (ES) cells that are found in blastocysts, and adult stem cells that are found in adult tissues. In a developing embryo, stem cells can differentiate into all of the specialized embryonic tissues. In adult organisms, stem cells and progenitor cells act as a repair system for the body, replenishing specialized cells, but also maintain the normal turnover of regenerative organs, such as blood, skin or intestinal tissues. Pluripotent stem cells can differentiate into cells derived from any of the three germ layers.

Stem cells are generally classified by their developmental potential as: (1) “totipotent,” meaning able to give rise to all embryonic and extraembryonic cell types; (2) “pluripotent,” meaning able to give rise to all embryonic cell types; (3) “multipotent,” meaning able to give rise to a subset of cell lineages, but all within a particular tissue, organ, or physiological system (for example, hematopoietic stem cells (HSCs) can produce progeny that include HSCs (self-renewal), blood cell restricted oligopotent progenitors and the cell types and elements (e.g., platelets) that are normal components of the blood); (4) “oligopotent,” meaning able to give rise to a more restricted subset of cell lineages than multipotent stem cells; and (5) “unipotent,” meaning able to give rise to a single cell lineage (e.g., spermatogenic stem cells).

“Self-renewal” refers to the ability of a cell to divide and generate at least one daughter cell with the identical (e.g., self-renewing) characteristics of the parent cell. The second daughter cell may commit to a particular differentiation pathway. For example, a self-renewing hematopoietic stem cell divides and forms one daughter stem cell and another daughter cell committed to differentiation in the myeloid or lymphoid pathway. In contrast, a committed progenitor cell has typically lost the self-renewal capacity, and upon cell division produces two daughter cells that display a more differentiated (i.e., restricted) phenotype. True hematopoietic stem cells have the ability to regenerate long term multi-lineage hematopoiesis (e.g., “long-term engraftment”) in individuals receiving a bone marrow or umbilical cord blood transplant, as described herein.

Hematopoietic stem cells are traditionally identified as being lineage marker negative, Sca1-positive, cKit-positive (or LSK cells), CD34-negative, Flk2-negative, CD48-negative, and CD150 positive. HSCs give rise to “multipotent progenitor cells” or “hematopoietic progenitor cells,” which, as the terms are used herein, refer to a more differentiated subset of multipotent stem cells that while committed to the hematopoietic cell lineage generally do not self-renew. The terms “hematopoietic progenitor cells” or “multi-potent progenitor cells” (MPPs) encompass short term hematopoietic stem cells (also known as ST-HSCs, which are lineage marker negative, Sca1-positive, cKit-positive, CD34-positive, and Flk2-negative); common myeloid progenitor cells (CMPs); lymphoid-primed progenitor cells (LMPPs), granulocyte-monocyte progenitor cells (GMPs), and megakaryocyte-erythrocyte progenitor cells (MEPs). Hematopoietic stem cells subsets are sometimes also identified and discriminated on the basis of additional cell-surface marker phenotypes, such as by using combinations of members of the SLAM family, or the “SLAM phenotype,” such as, long-term multi-lineage repopulating and self-renewing hematopoietic stem cells (HSCs): CD150⁺CD48⁻CD244⁻; MPPs: CD150⁻CD48⁻CD244⁺; lineage-restricted progenitor cells (LRPs): CD150⁻CD48⁺CD244⁺; common myeloid progenitor cells (CMP): lin⁻SCA-1⁻c-kit⁺CD34⁺CD16/32^{mid}; granulocyte-macrophage progenitor (GMP): lin⁻SCA-1⁻c-kit⁺CD34⁺CD16/32^{hi}; and megakaryocyte-erythroid progenitor (MEP): lin⁻SCA-1⁻c-kit⁺CD34⁺CD16/32^{low}.

Accordingly, using the compositions, constructs, methods, and kits comprising the HSC reprogramming factors or HSC inducing factors described herein, induced hematopoietic stem cells or iHSCs can be generated that are multipotent and capable of differentiating into all the blood or immune cell types of the hematopoietic system, including, but not limited to, myeloid cells (monocytes and macrophages, neutrophils, basophils, eosinophils, erythrocytes, megakaryocytes/platelets, dendritic cells), and lymphoid lineages (T-cells, B-cells, NKT-cells, NK-cells), and which have multi-lineage hematopoietic differentiation potential and sustained self-renewal activity. In some embodiments of the compositions, constructs, methods, and kits comprising the HSC reprogramming factors or HSC inducing factors described herein, cells are dedifferentiated into one or more other hematopoietic progenitor cells types, such as short term hematopoietic stem cells, common myeloid progenitor cells, common lymphoid progenitor cells, lymphoid-primed progenitor cells, granulocyte-monocyte progenitor cells, and megakaryocyte-erythrocyte progenitor cells.

The successful identification of HSC inducing factors capable of reprogramming committed blood cells to induced HSCs, as described herein, can advance our basic understanding of HSC biology in a number of ways. Despite the fact that HSCs are the most well characterized tissue-specific stem cells, surprisingly little is known about the molecular mechanisms involved in regulating their central properties of self-renewal and multi-potency. Identification of factors capable of imparting self-renewal and multi-lineage potential onto otherwise non-self-renewing, lineage-restricted cells, as described herein, provide important insights into the molecular basis of these fundamental attributes and provide strategies on how best to therapeutically manipulate HSCs. Further, mature blood cell production is an ongoing process requiring profound homeostatic control mechanisms—the primary level of which resides with HSCs. Since hematopoietic malignancies arise through deregulation of homeostatic control mechanisms, identification of regulators responsible for specifying HSC function, such as the HSC inducing factors described herein, can also provide important insights into how homeostasis is regulated by stem cells, and in turn, how deregulation of such processes manifest in disease. Functional conservation of reprogramming factors between species is well-documented indicating that it the methods and compositions described herein are applicable for reprogramming human blood cells to induced HSCs, using homologues of the murine reprogramming factors described herein. The ability to derive functional human induced HSCs in such a manner represents a new experimental paradigm for deriving these important cells that can be translated clinically, or used to model hematopoietic diseases. Because one mechanism in which lineage specification has been shown to occur is by the active suppression of alternative fates, by identifying factors involved in re-establishing core HSC properties, factors that function by suppressing differentiation programs can also be identified. If so, identification of such factors could provide fundamental insights into hematopoietic lineage specification. Transcription factors play a critical role in the specification of all cell types during development. The success of reprogramming strategies using transcription factor-mediated de-differentiation of cells indicates that it is equally plausible to direct the differentiation of pluripotent ES/iPS cells to specific fates using such factors. Accordingly, using the HSC inducing factors identified herein, directed differentiation of ES/iPS cells to a definitive HSC fate by expression of the HSC-enriched transcription factors can be achieved.

The combinatorial introduction of HSC-enriched TFs into downstream progenitors and screening for the introduction of stem cell properties onto these committed cells in vivo has identified a core set of TFs, referred to herein as “HSC inducing factors” or “HSC reprogramming factors” able to mediate the reprogramming of committed cells back to an induced hematopoietic stem cell (iHSC) state. With the approaches described herein, advantage can be taken of the fact that HSCs are the only cells in the hematopoietic system capable of giving rise to long-term (>4 months) multi-lineage reconstitution in transplantation assays, whereas committed progenitors reconstitute recipient mice only transiently with restricted lineage potential depending upon their stage of differentiation. Only progenitors that have been successfully reprogrammed to an induced hematopoietic stem cell state are able to give rise to long-term multi-lineage reconstitution in transplant recipients, using the compositions, methods, and kits described herein.

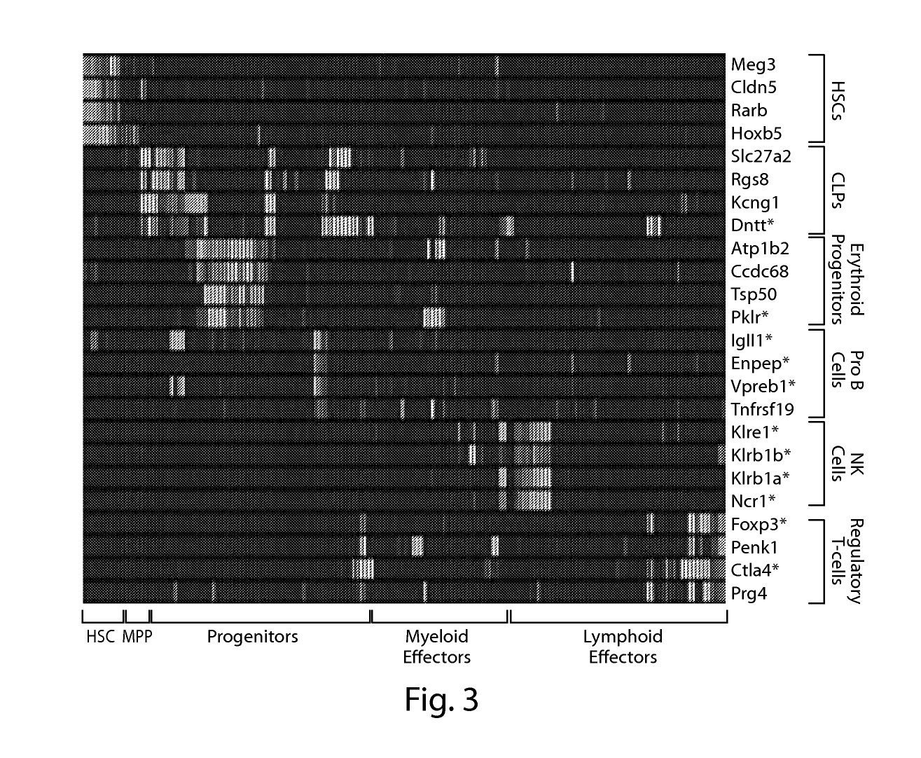

To realize the goal of identifying transcription factors specifically expressed in HSCs within the hematopoietic system, a comprehensive system-wide approach was undertaken in which expression profiles of 40 FACS purified hematopoietic cell types, representing the vast majority of hematopoietic stem, progenitor and effector cells, were generated and compiled (FIG. 1). Since the success of the results described herein require a detailed knowledge of the molecular attributes of HSCs, the focus has been on defining these by expression profiling of purified HSCs from diverse settings ranging from steady state hematopoiesis through different stages of ontogeny (fetal development through to old age). Throughout the work described herein, HSCs are fluorescence activated cell sorted (FACS) purified by stringent cell surface phenotype, and defined through functional criteria (FIGS. 1-2). In total, 46 expression profiles for HSCs were generated, which lends enormous statistical power to the analyses described herein. In total, 248 expression profiles of hematopoietic populations have been generated and normalized into a single database (referred to as the “hematopoietic expression database”) (FIG. 3).

Using the databases described herein, transcriptional factors (TFs) with HSC-enriched expression have been identified. In some embodiments of the aspects described herein, in addition to the factors with strict HSC-enriched expression, TFs involved in specifying hematopoietic fate during fetal development such as SCL/TAL1, RUNX1, HOXB4, and LMO2, can be used as HSC inducing factors, even though they do not exhibit particularly HSC-specific expression in the adult. In total, as described herein, over 40 TFs that can be used in various combinations as “HSC inducing factors,” as the term is used herein, have been identified and the expression profiles of each have been confirmed by qRT-PCR.

The production of cells having an increased developmental potential (e.g., iHSCs) is generally achieved by the introduction of nucleic acid sequences encoding genes identified herein as “HSC inducing factors” into an adult, somatic cell, preferably, in some embodiments, a more differentiated cell of the hematopoietic lineage. Typically, nucleic acids encoding the HSC inducing factors, e.g., DNA or RNA, or constructs thereof, are introduced into a cell, using viral vectors or without viral vectors, via one or repeated transfections, and the expression of the gene products and/or translation of the RNA molecules result in cells that are morphologically, biochemically, and functionally similar to HSCs, as described herein. As used herein, “reprogramming” refers to a process of driving a cell to a state with higher developmental potential, i.e., backwards, to a less differentiated state. In some embodiments of the compositions, methods, and kits described herein, reprogramming encompasses a complete or partial reversion of the differentiation state to that of a cell having a multipotent state. In some embodiments of the compositions, methods, and kits described herein, reprogramming encompasses a complete or partial reversion of the differentiation state to that of a cell having the state of a hematopoietic progenitor cell, such as a CMP, a CLP, etc. The hematopoietic stem cells induced by the compositions, methods, and kits described herein are termed herein as “induced hematopoietic stem cells,” “iHS cells,” or “iHSCs.” Compositions comprising amino acid or nucleic acid sequences or expression vectors thereof encoding these HSC inducing factors are referred to herein as “HSC inducing compositions.”

As demonstrated herein, over 40 transcription factors were identified that can be introduced into a cell in various combinations as “HSC inducing factors” to generate induced hematopoietic stem cells or iHSCs that are multipotent and capable of differentiating into all or a majority the blood or immune cell types of the hematopoietic system, including, but not limited to, myeloid cells (monocytes and macrophages, neutrophils, basophils, eosinophils, erythrocytes, megakaryocytes/platelets, dendritic cells), and lymphoid lineages (T-cells, B-cells, NKT-cells, NK-cells), and which have multi-lineage hematopoietic differentiation potential and sustained self-renewal activity. Thus, provided herein, in some aspects, are HSC inducing factors and combinations thereof comprising the genes listed in Table 1, which also provides exemplary sequences for making the identified proteins:

TABLE 1
HSC Inducing Factors

| GENE | Human mRNA | SEQ | Murine mRNA | SEQ |
|---|---|---|---|---|
| NAME | REF SEQ | ID NOs: | REF SEQ | ID NOs: |
| CDKN1C | NM_000076.2 | SEQ ID | NM_001161624.1 | SEQ ID |
| | | NO: 1 | | NO: 47 |
| DNMT3B | NM_001207055.1 | SEQ ID | NM_001003960.4 | SEQ ID |
| | | NO: 2 | | NO: 48 |
| EGR1 | NM_001964.2 | SEQ ID | NM_133659.2 | SEQ ID |
| | | NO: 3 | | NO: 49 |
| ETV6 | NM_001987.4 | SEQ ID | NM_007961.3 | SEQ ID |
| | | NO: 4 | | NO: 50 |
| EVI1 | NM_001105078.3 | SEQ ID | NM_007963.2 | SEQ ID |
| | | NO: 5 | | NO: 51 |
| GATA2 | NM_032638.4 | SEQ ID | NM_008090.5 | SEQ ID |
| | | NO: 6 | | NO: 52 |
| GFI1B | NM_001135031.1 | SEQ ID | NM_001160406.1 | SEQ ID |
| | | NO: 7 | | NO: 53 |
| GLIS2 | NM_032575.2 | SEQ ID | NM_031184.3 | SEQ ID |
| | | NO: 8 | | NO: 54 |
| HLF | NM_002126.4 | SEQ ID | NM_172563.3 | SEQ ID |
| | | NO: 9 | | NO: 55 |
| HMGA2 | NM_003483.4 | SEQ ID | NM_010441.2 | SEQ ID |
| | | NO: 10 | | NO: 56 |
| HOXA5 | NM_019102.3 | SEQ ID | NM_010453.5 | SEQ ID |
| | | NO: 11 | | NO: 57 |
| HOXA9 | NM_152739.3 | SEQ ID | NM_010456.2 | SEQ ID |
| | | NO: 12 | | NO: 58 |
| HOXB3 | NM_002146.4 | SEQ ID | NM_001079869.1 | SEQ ID |
| | | NO: 13 | | NO: 59 |
| HOXB4 | NM_024015.4 | SEQ ID | NM_010459.7 | SEQ ID |
| | | NO: 14 | | NO: 60 |
| HOXB5 | NM_002147.3 | SEQ ID | NM_008268.2 | SEQ ID |
| | | NO: 15 | | NO: 61 |
| IGF2BP2 | NM_001007225.1 | SEQ ID | NM_183029.2 | SEQ ID |
| | | NO: 16 | | NO: 62 |
| IKZF2 | NM_001079526.1 | SEQ ID | NM_011770.4 | SEQ ID |
| | | NO: 17 | | NO: 63 |
| KLF12 | NM_007249.4 | SEQ ID | NM_010636.3 | SEQ ID |
| | | NO: 18 | | NO: 64 |
| KLF4 | NM_004235.4 | SEQ ID | NM_010637.3 | SEQ ID |
| | | NO: 19 | | NO: 65 |
| KLF9 | NM_001206.2 | SEQ ID | NM_010638.4 | SEQ ID |
| | | NO: 20 | | NO: 66 |
| LMO2 | NM_005574.3 | SEQ ID | NM_001142336.1 | SEQ ID |
| | | NO: 21 | | NO: 67 |
| MEIS1 | NM_002398.2 | SEQ ID | NM_00193271.1 | SEQ ID |
| | | NO: 22 | | NO: 68 |
| MSI2 | NM_138962.2 | SEQ ID | NM_054043.3 | SEQ ID |
| | | NO: 23 | | NO: 69 |
| MYCN | NM_005378.4 | SEQ ID | NM_008709 | SEQ ID |
| | | NO: 24 | | NO: 70 |
| NAP1L3 | NM_004538.5 | SEQ ID | NM_138742.1 | SEQ ID |
| | | NO: 25 | | NO: 71 |
| NDN | NM_004538.5 | SEQ ID | NM_010882.3 | SEQ ID |
| | | NO: 26 | | NO: 72 |
| NFIX | NM_001271044.1 | SEQ ID | NM_001081981.1 | SEQ ID |
| | | NO: 27 | | NO: 73 |
| NKX2-3 | NM_145285.2 | SEQ ID | NM_008699.2 | SEQ ID |
| | | NO: 28 | | NO: 74 |
| NR3C2 | NM_000901.4 | SEQ ID | NM_001083906.1 | SEQ ID |
| | | NO: 29 | | NO: 75 |
| PBX1 | NM_001204961.1 | SEQ ID | NM_008783.2 | SEQ ID |
| | | NO: 30 | | NO: 76 |
| PRDM16 | NM_199454.2 | SEQ ID | NM_001177995.1 | SEQ ID |
| | | NO: 31 | | NO: 77 |
| PRDM5 | NM_018699.2 | SEQ ID | NM_027547.2 | SEQ ID |
| | | NO: 32 | | NO: 78 |
| RARB | NM_000965.3 | SEQ ID | NM_011243.1 | SEQ ID |
| | | NO: 33 | | NO: 79 |
| RBBP6 | NM_006910.4 | SEQ ID | NM_011247.2 | SEQ ID |
| | | NO: 34 | | NO: 80 |
| RBPMS | NM_001008712.1 | SEQ ID | NM_019733.2 | SEQ ID |
| | | NO: 35 | | NO: 81 |
| RUNX1 | NM_001001890.2 | SEQ ID | NM_001111021.1 | SEQ ID |
| | | NO: 36 | | NO: 82 |
| RUNX1T1 | NM_001198625.1 | SEQ ID | NM_009822.2 | SEQ ID |
| | | NO: 37 | | NO: 83 |
| SMAD6 | NM_001142861.2 | SEQ ID | NM_008542.3 | SEQ ID |
| | | NO: 38 | | NO: 84 |
| TAL1 | NM_003189.2 | SEQ ID | NM_011527.2 | SEQ ID |
| | | NO: 39 | | NO: 85 |
| TCF15 | NM_004609.3 | SEQ ID | NM_009328.2 | SEQ ID |
| | | NO: 40 | | NO: 86 |
| VDR | NM_000376.2 | SEQ ID | NM_009504.4 | SEQ ID |
| | | NO: 41 | | NO: 87 |
| ZFP37 | NM_003408.1 | SEQ ID | NM_009554.3 | SEQ ID |
| | | NO: 42 | | NO: 88 |
| ZFP467 | NM_207336.1 | SEQ ID | NM_001085415.1 | SEQ ID |
| | | NO: 43 | | NO: 89 |
| ZFP521 | NM_015461.2 | SEQ ID | NM_145492.4 | SEQ ID |
| | | NO: 44 | | NO: 90 |
| ZFP532 | NM_018181.4 | SEQ ID | NM_207255.2 | SEQ ID |
| | | NO: 45 | | NO: 91 |
| ZFP612 | NM_145911.1 | SEQ ID | NM_175480.4 | SEQ ID |
| | | NO: 46 | | NO: 92 |

In some embodiments, polypeptide variants or family members having the same or a similar activity as the reference polypeptide encoded by the sequences provided in Table 1 can be used in the compositions, methods, and kits described herein. Generally, variants of a particular polypeptide encoding a HSC inducing factor for use in the compositions, methods, and kits described herein will have at least about 75%, at least about 80%, at least about 85%, at least about 90%, at least about 91%, at least about 92%, at least about 93%, at least about 94%, at least about 95%, at least about 96%, at least about 97%, at least about 98%, at least about 99% or more sequence identity to that particular reference polynucleotide or polypeptide as determined by sequence alignment programs and parameters described herein and known to those skilled in the art.

Accordingly, in some embodiments, the HSC inducing factors for use in the compositions, methods, and kits described herein, are selected from the group consisting of: CDKN1C, DNMT3B, EGR1, ETV6, EVI1, GATA2, GFI1B, GLIS2, HLF, HMGA2, HOXA5, HOXA9, HOXB3, HOXB4, HOXB5, IGF2BP2, IKZF2, KLF12, KLF4, KLF9, LMO2, MEIS1, MSI2, MYCN, NAP1L3, NDN, NFIX, NKX2-3, NR3C2, PBX1, PRDM16, PRDM5, RARB, RBBP6, RBPMS, RUNX1, RUNX1T1, SMAD6, TAL1, TCF15, VDR, ZFP37, ZFP467, ZFP521, ZFP532, and ZFP612 (SEQ ID NOs: 1-46).

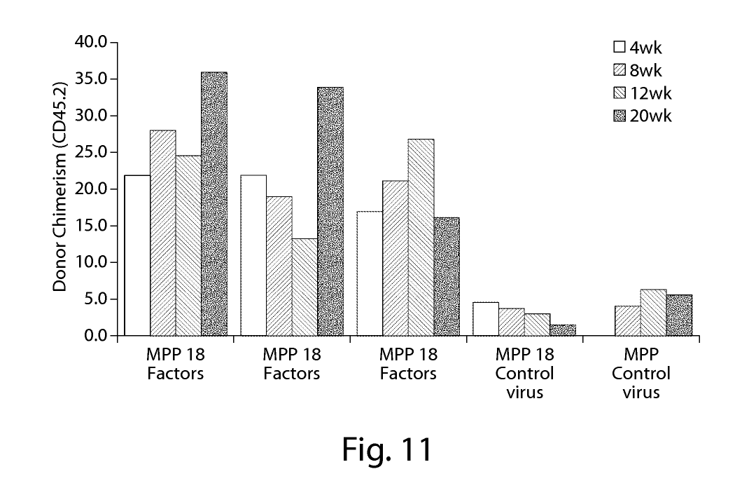

As demonstrated herein, for example at FIG. 11, exposure to 18 transcription factors from the genes listed in Table 1 provided MPP cells with robust long-term, multi-lineage engraftment properties, characteristic of HSCs, in vivo. Accordingly, in some embodiments of the compositions, methods, and kits described herein, the HSC inducing factors are selected from: HLF, MYCN, MEIS1, IRF6, CDKN1C, NFIX, DNMT3B, ZFP612, PRDM5, HOXB4, LMO2, NKX2-3, RARB, NDN, NAP1L3, RUNX1T1, ZFP467, and ZFP532. Another grouping is a core 6 factors (Runx1t1, HLF, PRDM5, PBX1, LMO2, and ZFP37), and 8 factors (the 6 factors plus MEIS1, MYCN).

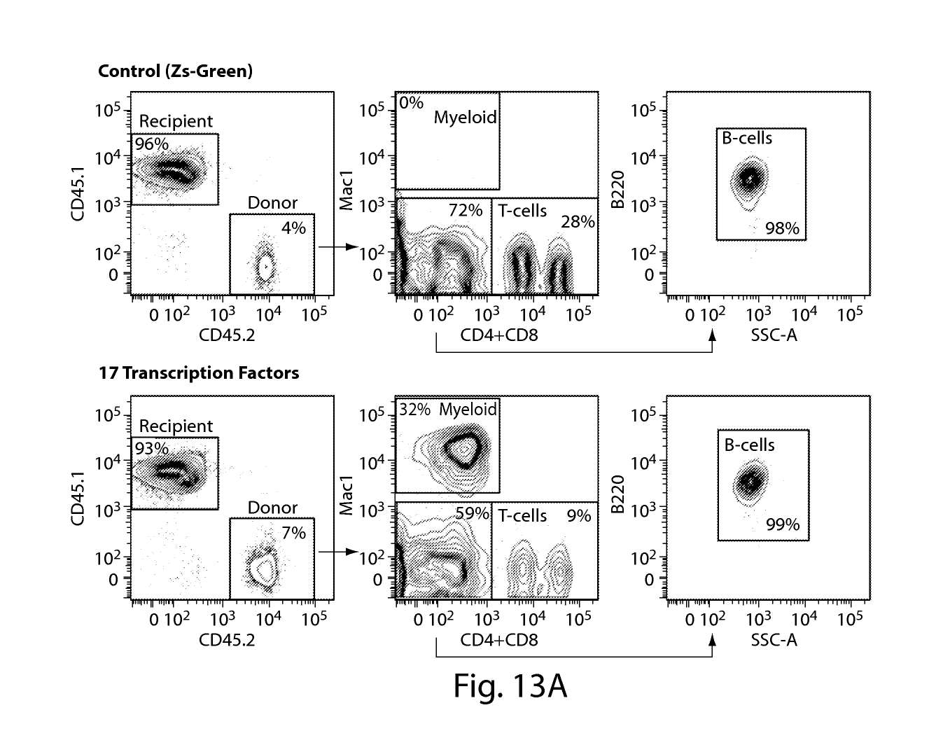

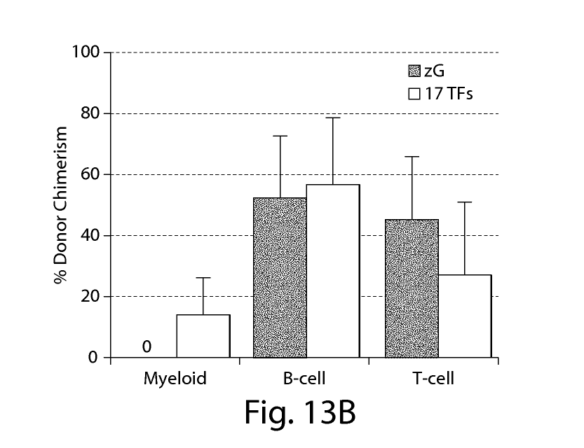

As demonstrated herein, for example at FIGS. 13A-13B, exposure to 17 transcription factors from the genes listed in Table 1 provided MPP cells with robust long-term, multi-lineage engraftment properties, characteristic of HSCs, in vivo. Accordingly, in some embodiments of the compositions, methods, and kits described herein, the HSC inducing factors are selected from: HLF, MYCN, MEIS1, IRF6, NFIX, DNMT3B, ZFP612, PRDM5, HOXB4, LMO2, NKX2-3, RARB, NDN, NAP1L3, RUNX1T1, ZFP467, and ZFP532.

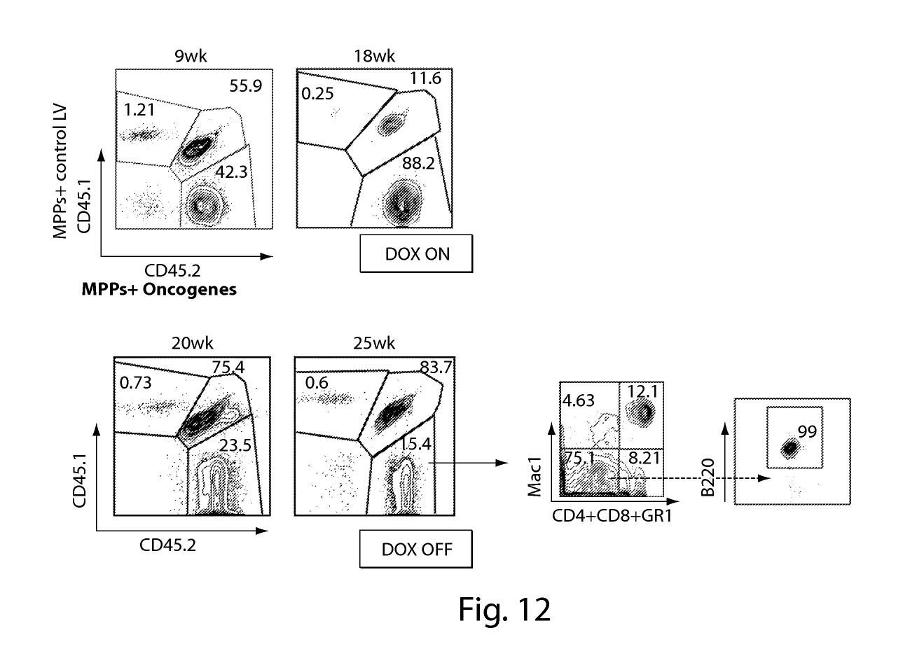

As demonstrated herein, for example at FIG. 12, exposure to 9 transcription factors from the genes listed in Table 1 provided MPP cells with robust long-term, multi-lineage engraftment properties, characteristic of HSCs, in vivo. Accordingly, in some embodiments of the compositions, methods, and kits described herein, the HSC inducing factors are selected from: EVI-1, GLIS2, HOXB5, HOXA9, HLF, MEIS1, MYCN, PRDM16, and RUNX1.

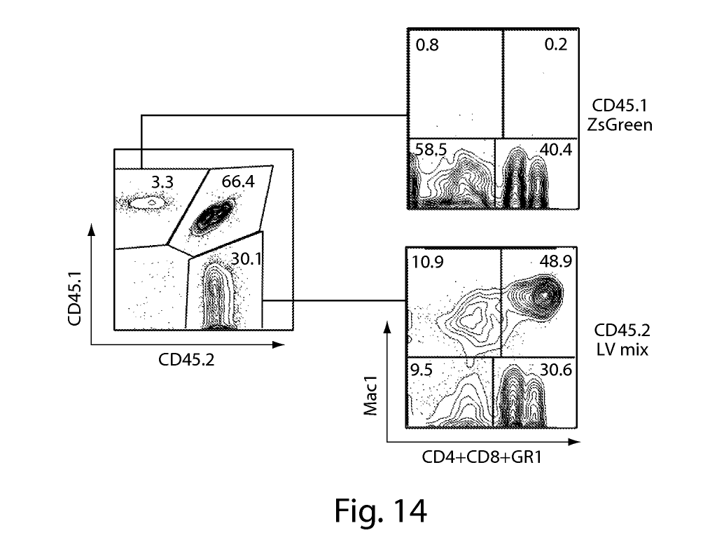

As demonstrated herein, for example at FIG. 14, exposure to 8 transcription factors from the genes listed in Table 1 provided MPP cells with robust long-term, multi-lineage engraftment properties, characteristic of HSCs, in vivo. In some embodiments of the compositions, methods, and kits described herein, the HSC inducing factors are selected from: RUNX1T1, HLF, ZFP467, RBPMS, HOXB5, NAP1L3, MSI2, and IRF6.

In some embodiments of the aspects described herein, the HSC inducing factors for use with the compositions, methods, and kits comprise, consist essentially of, or consist of HLF, RUNX1T1, PBX1, LMO2, PRDM5, ZFP37, MYCN, MSI2, NKX2-3, MEIS1, and RBPMS. As demonstrated herein, the use of these 11 HSC inducing factors together, also referred to herein as “Combination 7” or “C7,” resulted in increased colony formation, altered lineage potential, and multi-lineage reconstitution in vivo, from CMP cells or ProPreB cells. In addition, this combination was shown to have serial long-term transplantation potential in vivo. Accordingly, in some embodiments of the compositions, methods, and kits described herein, the HSC inducing factors are selected from HLF, RUNX1T1, PBX1, LMO2, PRDM5, ZFP37, MYCN, MSI2, NKX2-3, MEIS1, and RBPMS.

In some embodiments of the aspects described herein, the HSC inducing factors for use with the compositions, methods, and kits comprise, consist essentially of, or consist of HLF, RUNX1T1, ZFP37, PBX1, LMO2, and PRDM5. As demonstrated herein, the use of these 6 HSC inducing factors together, also referred herein as “Combination 6” or “C6,” was able to reprogram ProPreB or CMP cells into cells capable of giving rise to multi-lineage reconstitution in vivo. Accordingly, in some embodiments of the compositions, methods, and kits described herein, the HSC inducing factors are selected from HLF, ZFP37, RUNX1T1, PBX1, LMO2, and PRDM5. In some embodiments, the compositions, methods, and kits described herein can further comprise one or more of the HSC inducing factors PRDM16, ZFP467, and VDR.

In some embodiments of the aspects described herein, the HSC inducing factors for use with the compositions, methods, and kits comprise, consist essentially of, or consist of ZFP467, PBX1, HOXB4, and MSI2. As demonstrated herein, the use of these HSC inducing factors together, also referred herein as “Combination 1” or “C1,” was able to reprogram ProPreB cells to myeloid cells. Accordingly, in some embodiments of the compositions, methods, and kits described herein, the HSC inducing factors are selected from ZFP467, PBX1, HOXB4, and MSI2. In some embodiments, the compositions, methods, and kits described herein can further comprise one or more of the HSC inducing factors HLF, LMO2, PRDM16, and ZFP37.

In some embodiments of the aspects described herein, the HSC inducing factors for use with the compositions, methods, and kits comprise, consist essentially of, or consist of MYCN, MSI2, NKX2-3, and RUNX1T1. As demonstrated herein, the use of these HSC inducing factors together, also referred herein as “Combination 2” or “C2,” was able to reprogram ProPreB cells to iHSCs. Accordingly, in some embodiments of the compositions, methods, and kits described herein, the HSC inducing factors are selected from MYCN, MSI2, NKX2-3, and RUNX1T1. In some embodiments, the compositions, methods, and kits described herein can further comprise one or more of the HSC inducing factors HOBX5, HLF, ZFP467, HOXB3, LMO2, PBX1, ZFP37, and ZFP521.

In some embodiments of the aspects described herein, the HSC inducing factors for use with the compositions, methods, and kits comprise, consist essentially of, or consist of HOXB4, PBX1, LMO2, ZFP612, and ZFP521. As demonstrated herein, the use of these HSC inducing factors together, also referred herein as “Combination 3” or “C3,” was able to promote the proliferation and survival of ProPreB cells. Accordingly, in some embodiments of the compositions, methods, and kits described herein, the HSC inducing factors are selected from HOXB4, PBX1, LMO2, ZFP612, and ZFP521. In some embodiments, the compositions, methods, and kits described herein can further comprise one or more of the HSC inducing factors KLF12, HLF, and EGR1.

In some embodiments of the aspects described herein, the HSC inducing factors for use with the compositions, methods, and kits comprise, consist essentially of, or consist of MEIS1, RBPMS, ZFP37, RUNX1T1, and LMO2. As demonstrated herein, the use of these HSC inducing factors together, also referred herein as “Combination 4” or “C4,” was able to reprogram CMP cells to iHSCs. Accordingly, in some embodiments of the compositions, methods, and kits described herein, the HSC inducing factors are selected from MEIS1, RBPMS, ZFP37, RUNX1T1, and LMO2. In some embodiments, the compositions, methods, and kits described herein can further comprise one or more of the HSC inducing factors KLF12 and HLF.

In some embodiments of the aspects described herein, the HSC inducing factors for use with the compositions, methods, and kits comprise, consist essentially of, or consist of ZFP37, HOXB4, LMO2, and HLF. As demonstrated herein, the use of these HSC inducing factors together, also referred herein as “Combination 5” or “C5,” was able to reprogram the fates of CMP and ProPreB cells. Accordingly, in some embodiments of the compositions, methods, and kits described herein, the HSC inducing factors are selected from ZFP37, HOXB4, LMO2, and HLF. In some embodiments, the compositions, methods, and kits described herein can further comprise one or more of the HSC inducing factors MYCN, ZFP467, NKX2-3, PBX1, and KLF12ZFP37.

In some embodiments of the compositions, methods, and kids provided herein, the number of HSC inducing factors used or selected to generate iHSCs from a starting somatic cell, such as a fibroblast cell or hematopoietic lineage cell, is at least three. In some embodiments, the number of HSC inducing factors used or selected is at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, at least twenty, or more.

Also provided herein, in various aspects of the compositions, methods, and kits, are isolated amino acid sequences, and isolated DNA or RNA nucleic acid sequences encoding one or more HSC inducing factors for use in making iHSCS.

In some embodiments of the compositions, methods, and kits described herein, the nucleic acid sequence or construct encoding the HSC inducing factor(s), such as HLF, RUNX1T1, PBX1, LMO2, PRDM5, ZFP37, MYCN, MSI2, NKX2-3, MEIS1, and RBPMS, is inserted or operably linked into a suitable expression vector for transfection of cells using standard molecular biology techniques. As used herein, a “vector” refers to a nucleic acid molecule, such as a dsDNA molecule that provides a useful biological or biochemical property to an inserted nucleotide sequence, such as the nucleic acid constructs or replacement cassettes described herein. Examples include plasmids, phages, autonomously replicating sequences (ARS), centromeres, and other sequences that are able to replicate or be replicated in vitro or in a host cell, or to convey a desired nucleic acid segment to a desired location within a host cell. A vector can have one or more restriction endonuclease recognition sites (whether type I, II or IIs) at which the sequences can be cut in a determinable fashion without loss of an essential biological function of the vector, and into which a nucleic acid fragment can be spliced or inserted in order to bring about its replication and cloning. Vectors can also comprise one or more recombination sites that permit exchange of nucleic acid sequences between two nucleic acid molecules. Vectors can further provide primer sites, e.g., for PCR, transcriptional and/or translational initiation and/or regulation sites, recombination signals, replicons, additional selectable markers, etc. A vector can further comprise one or more selectable markers suitable for use in the identification of cells transformed with the vector.

Accordingly, in some aspects, provided herein are hematopoietic stem cell (HSC) inducing compositions comprising one or more expression vectors encoding at least one, two, three, four, five, six, seven, eight or more HSC inducing factors selected from: CDKN1C, DNMT3B, EGR1, ETV6, EVI1, GATA2, GFI1B, GLIS2, HLF, HMGA2, HOXA5, HOXA9, HOXB3, HOXB4, HOXB5, IGF2BP2, IKZF2, KLF12, KLF4, KLF9, LMO2, MEIS1, MSI2, MYCN, NAP1L3, NDN, NFIX, NKX2-3, NR3C2, PBX1, PRDM16, PRDM5, RARB, RBBP6, RBPMS, RUNX1, RUNX1T1, SMAD6, TAL1, TCF15, VDR, ZFP37, ZFP467, ZFP521, ZFP532, and ZFP612.

In some embodiments of these aspects and all such aspects described herein, the at least one, two, three, four, or more HSC inducing factors are HLF, RUNX1T1, PBX1, LMO2, PRDM5, ZFP37, MYCN, MSI2, NKX2-3, MEIS1, and RBPMS.

In some embodiments of these aspects and all such aspects described herein, the at least one, two, three, four, or more HSC inducing factors are HLF, RUNX1T1, ZFP37, PBX1, LMO2, and PRDM5.

Also provided herein in some aspects are hematopoietic stem cell (HSC) inducing compositions comprising one or more expression vectors comprising: a nucleic acid sequence encoding HLF; a nucleic acid sequence encoding RUNX1T1; a nucleic acid sequence encoding ZFP37; a nucleic acid sequence encoding PBX1; a nucleic acid sequence encoding LMO2; and a nucleic acid sequence encoding PRDM5.

In some embodiments of these aspects and all such aspects described herein, the HSC inducing composition further comprises one or more of: a nucleic acid sequence encoding PRDM16; a nucleic acid sequence encoding ZFP467; and a nucleic acid sequence encoding VDR.

Also provided herein in some aspects are hematopoietic stem cell (HSC) inducing compositions comprising one or more expression vectors comprising: a nucleic acid sequence encoding HLF; a nucleic acid sequence encoding RUNX1T1; a nucleic acid sequence encoding PBX1; a nucleic acid sequence encoding LMO2; a nucleic acid sequence encoding PRDM5; a nucleic acid sequence encoding ZFP37; a nucleic acid sequence encoding MYCN; a nucleic acid sequence encoding MSI2; a nucleic acid sequence encoding NKX2-3; a nucleic acid sequence encoding MEIS1; and a nucleic acid sequence encoding RBPMS.

In some aspects, provided herein are hematopoietic stem cell (HSC) inducing compositions comprising one or more expression vectors comprising: a nucleic acid sequence encoding ZFP467; a nucleic acid sequence encoding PBX1; a nucleic acid sequence encoding HOXB4; and a nucleic acid sequence encoding MSI2.

In some embodiments of these aspects and all such aspects described herein, the HSC inducing composition further comprises one or more of: a nucleic acid sequence encoding HLF; a nucleic acid sequence encoding LMO2; a nucleic acid sequence encoding PRDM16; and a nucleic acid sequence encoding ZFP37.

Also provided herein in some aspects are hematopoietic stem cell (HSC) inducing compositions comprising one or more expression vectors comprising: a nucleic acid sequence encoding MYCN; a nucleic acid sequence encoding MSI2; a nucleic acid sequence encoding NKX2-3; and a nucleic acid sequence encoding RUNX1T1.

In some embodiments of these aspects and all such aspects described herein, the HSC inducing composition further comprises a nucleic acid sequence encoding HOXB5; a nucleic acid sequence encoding HLF; a nucleic acid sequence encoding ZFP467; a nucleic acid sequence encoding HOXB3; a nucleic acid sequence encoding LMO2; a nucleic acid sequence encoding PBX1; a nucleic acid sequence encoding ZFP37; and a nucleic acid sequence encoding ZFP521.

In other aspects, provided herein are hematopoietic stem cell (HSC) inducing compositions comprising one or more expression vectors composition comprising: a nucleic acid sequence encoding HOXB4; a nucleic acid sequence encoding PBX1; a nucleic acid sequence encoding LMO2; a nucleic acid sequence encoding ZFP467; and a nucleic acid sequence encoding ZFP521.

In some embodiments of these aspects and all such aspects described herein, the HSC inducing composition further comprises one or more of: a nucleic acid sequence encoding KLF12; a nucleic acid sequence encoding HLF; and a nucleic acid sequence encoding EGR1.

Also provided herein in some aspects are hematopoietic stem cell (HSC) inducing compositions comprising one or more expression vectors comprising: a nucleic acid sequence encoding MEIS1; a nucleic acid sequence encoding RBPMS; a nucleic acid sequence encoding ZFP37; a nucleic acid sequence encoding RUNX1T1; and a nucleic acid sequence encoding LMO2.

In some embodiments of these aspects and all such aspects described herein, the HSC inducing composition further comprises one or more of a sequence encoding KLF12; and a sequence encoding HLF.

Also provided herein in some aspects are hematopoietic stem cell (HSC) inducing compositions comprising one or more expression vectors comprising: a nucleic acid sequence encoding ZFP37; a nucleic acid sequence encoding HOXB4; a nucleic acid sequence encoding LMO2; and a nucleic acid sequence encoding HLF.

In some embodiments of these aspects and all such aspects described herein, the HSC inducing composition further comprises one or more of: a nucleic acid sequence encoding MYCN; a nucleic acid sequence encoding ZFP467; a nucleic acid sequence encoding NKX2-3; a nucleic acid sequence encoding PBX1; and a nucleic acid sequence encoding KLF4.

In some embodiments of the compositions, methods, and kits described herein, the expression vector is a viral vector. Some viral-mediated expression methods employ retrovirus, adenovirus, lentivirus, herpes virus, pox virus, and adeno-associated virus (AAV) vectors, and such expression methods have been used in gene delivery and are well known in the art.

In some embodiments of the compositions, methods, and kits described herein, the viral vector is a retrovirus. Retroviruses provide a convenient platform for gene delivery. A selected gene can be inserted into a vector and packaged in retroviral particles using techniques known in the art. The recombinant virus can then be isolated and delivered to target cells of the subject either in vivo or ex vivo. A number of retroviral systems have been described. See, e.g., U.S. Pat. No. 5,219,740; Miller and Rosman (1989) BioTechniques 7:980-90; Miller, A. D. (1990) Human Gene Therapy 1:5-14; Scarpa et al. (1991) Virology 180:849-52; Burns et al. (1993) Proc. Natl. Acad. Sci. USA 90:8033-37; Boris-Lawrie and Temin (1993) Curr. Opin. Genet. Develop. 3:102-09. In some embodiments of the compositions, methods, and kits described herein, the retrovirus is replication deficient. Retroviral vector systems exploit the fact that a minimal vector containing the 5′ and 3′ LTRs and the packaging signal are sufficient to allow vector packaging, infection and integration into target cells, provided that the viral structural proteins are supplied in trans in the packaging cell line. Fundamental advantages of retroviral vectors for gene transfer include efficient infection and gene expression in most cell types, precise single copy vector integration into target cell chromosomal DNA and ease of manipulation of the retroviral genome.

In some embodiments of the compositions, methods, and kits described herein, the viral vector is an adenovirus-based expression vector. Unlike retroviruses, which integrate into the host genome, adenoviruses persist extrachromosomally, thus minimizing the risks associated with insertional mutagenesis (Haj-Ahmad and Graham (1986) J. Virol. 57:267-74; Bett et al. (1993) J. Virol. 67:5911-21; Mittereder et al. (1994) Human Gene Therapy 5:717-29; Seth et al. (1994) J. Virol. 68:933-40; Barr et al. (1994) Gene Therapy 1:51-58; Berkner, K. L. (1988) BioTechniques 6:616-29; and Rich et al. (1993) Human Gene Therapy 4:461-76). Adenoviral vectors infect a wide variety of cells, have a broad host-range, exhibit high efficiencies of infectivity, direct expression of heterologous genes at high levels, and achieve long-term expression of those genes in vivo. The virus is fully infective as a cell-free virion so injection of producer cell lines is not necessary. With regard to safety, adenovirus is not associated with severe human pathology, and the recombinant vectors derived from the virus can be rendered replication defective by deletions in the early-region 1 (“E1”) of the viral genome. Adenovirus can also be produced in large quantities with relative ease. Adenoviral vectors for use in the compositions, methods, and kits described herein can be derived from any of the various adenoviral serotypes, including, without limitation, any of the over 40 serotype strains of adenovirus, such as serotypes 2, 5, 12, 40, and 41. The adenoviral vectors used herein are preferably replication-deficient and contain the HSC inducing factor of interest operably linked to a suitable promoter.

In some embodiments of the compositions, methods, and kits described herein, the nucleic acid sequences encoding the HSC inducing factor(s), such as HLF, RUNX1T1, PBX1, LMO2, PRDM5, ZFP37, MYCN, MSI2, NKX2-3, MEIS1, and RBPMS, are introduced or delivered using one or more inducible lentiviral vectors. Control of expression of HSC inducing factors delivered using one or more inducible lentiviral vectors can be achieved, in some embodiments, by contacting a cell having at least one HSC inducing factor in an expression vector under the control of or operably linked to an inducible promoter, with a regulatory agent (e.g., doxycycline) or other inducing agent. When using some types of inducible lentiviral vectors, contacting such a cell with an inducing agent induces expression of the HSC inducing factors, while withdrawal of the regulatory agent inhibits expression. When using other types of inducible lentiviral vectors, the presence of the regulatory agent inhibits expression, while removal of the regulatory agent permits expression. As used herein, the term “induction of expression” refers to the expression of a gene, such as an HSC inducing factor encoded by an inducible viral vector, in the presence of an inducing agent, for example, or in the presence of one or more agents or factors that cause endogenous expression of the gene in a cell.

In some embodiments of the aspects described herein, a doxycycline (Dox) inducible lentiviral system is used. Unlike retroviruses, lentiviruses are able to transduce quiescent cells making them amenable for transducing a wider variety of hematopoietic cell types. For example, the pHAGE2 lentivirus system has been shown to transduce primary hematopoietic progenitor cells with high efficiency. This vector also carries a reporter cassette (IRES Zs-Green) that enables evaluation of viral transduction efficiencies and purification of transduced cells by FACS. The ability to inducibly turn off introduced transcription factors, as demonstrated herein, is important since the HSC-enriched expression pattern of these TFs indicates their continued enforced expression in induced HSCs can impair differentiation to all lineages. Having an inducible system also allows ascertainment of the stability of the reprogrammed state and assess the establishment and fidelity of HSC transcriptional programs and epigenetic marks once enforced expression of reprogramming factors is lifted.

In some embodiments of the methods described herein, the nucleic acid sequences encoding the HSC inducing factor(s), such as HLF, RUNX1T1, PBX1, LMO2, PRDM5, ZFP37, MYCN, MSI2, NKX2-3, MEIS1, and RBPMS, are introduced or delivered using a non-integrating vector (e.g., adenovirus). While integrating vectors, such as retroviral vectors, incorporate into the host cell genome and can potentially disrupt normal gene function, non-integrating vectors control expression of a gene product by extra-chromosomal transcription. Since non-integrating vectors do not become part of the host genome, non-integrating vectors tend to express a nucleic acid transiently in a cell population. This is due in part to the fact that the non-integrating vectors are often rendered replication deficient. Thus, non-integrating vectors have several advantages over retroviral vectors including, but not limited to: (1) no disruption of the host genome, and (2) transient expression, and (3) no remaining viral integration products. Some non-limiting examples of non-integrating vectors for use with the methods described herein include adenovirus, baculovirus, alphavirus, picornavirus, and vaccinia virus. In some embodiments of the methods described herein, the non-integrating viral vector is an adenovirus. Other advantages of non-integrating viral vectors include the ability to produce them in high titers, their stability in vivo, and their efficient infection of host cells.

The phrases “operably linked,” “operatively positioned,” “operatively linked,” “under control,” and “under transcriptional control” indicate that a nucleic acid sequence, such as a sequence encoding an HSC inducing factor, is in a correct functional location and/or orientation in relation to a promoter and/or endogenous regulatory sequences, such that the promoter and/or endogenous regulatory sequences controls transcriptional initiation and/or expression of that sequence.

The terms “promoter” or “promoter sequence,” as used herein, refer to a nucleic acid sequence that regulates the expression of another nucleic acid sequence by driving RNA polymerase-mediated transcription of the nucleic acid sequence, which can be a heterologous target gene, such as a sequence encoding an HSC inducing factor. A promoter is a control region of a nucleic acid sequence at which initiation and rate of transcription of the remainder of a nucleic acid sequence are controlled. A promoter can also contain one or more genetic elements at which regulatory proteins and molecules can bind. Such regulatory proteins include RNA polymerase and other transcription factors. Accordingly, a promoter can be said to “drive expression” or “drive transcription” of the nucleic acid sequence that it regulates, such as a sequence encoding an HSC inducing factor.

Nucleic acid constructs and vectors for use in generating iHSCs in the compositions, methods, and kits described herein can further comprise, in some embodiments, one or more sequences encoding selection markers for positive and negative selection of cells. Such selection marker sequences can typically provide properties of resistance or sensitivity to antibiotics that are not normally found in the cells in the absence of introduction of the nucleic acid construct. A selectable marker can be used in conjunction with a selection agent, such as an antibiotic, to select in culture for cells expressing the inserted nucleic acid construct. Sequences encoding positive selection markers typically provide antibiotic resistance, i.e., when the positive selection marker sequence is present in the genome of a cell, the cell is sensitive to the antibiotic or agent. Sequences encoding negative selection markers typically provide sensitivity to an antibiotic or agent, i.e., when the negative selection marker is present in the genome of a cell, the cell is sensitive to the antibiotic or agent.

Nucleic acid constructs and vectors for use in making iHSCs in the compositions, methods, and kits thereof described herein can further comprise, in some embodiments, other nucleic acid elements for the regulation, expression, stabilization of the construct or of other vector genetic elements, for example, promoters, enhancers, TATA-box, ribosome binding sites, IRES, as known to one of ordinary skill in the art.

In some embodiments of the compositions, methods, and kits described herein, the HSC inducing factor(s), such as HLF, RUNX1T1, PBX1, LMO2, PRDM5, ZFP37, MYCN, MSI2, NKX2-3, MEIS1, and RBPMS, are provided as synthetic, modified RNAs, or introduced or delivered into a cell as a synthetic, modified RNA, as described in US Patent Publication 2012-0046346-A1, the contents of which are herein incorporated by reference in their entireties. In those embodiments where synthetic, modified RNAs are used to reprogram cells to iHSCs according to the methods described herein, the methods can involve repeated contacting of the cells or involve repeated transfections of the synthetic, modified RNAs encoding HSC inducing factors, such as for example, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, or more transfections.

In addition to one or more modified nucleosides, the modified mRNAs for use in the compositions, methods, and kits described herein can comprise any additional modifications known to one of skill in the art and as described in US Patent Publications 2012-0046346-A1 and 20120251618A1, and PCT Publication WO 2012/019168. Such other components include, for example, a 5′ cap (e.g., the Anti-Reverse Cap Analog (ARCA) cap, which contains a 5′-5′-triphosphate guanine-guanine linkage where one guanine contains an N7 methyl group as well as a 3′-O-methyl group; caps created using recombinant Vaccinia Virus Capping Enzyme and recombinant 2′-O-methyltransferase enzyme, which can create a canonical 5′-5′-triphosphate linkage between the 5′-most nucleotide of an mRNA and a guanine nucleotide where the guanine contains an N7 methylation and the ultimate 5′-nucleotide contains a 2′-O-methyl generating the Cap1 structure); a poly(A) tail (e.g., a poly-A tail greater than 30 nucleotides in length, greater than 35 nucleotides in length, at least 40 nucleotides, at least 45 nucleotides, at least 55 nucleotides, at least 60 nucleotide, at least 70 nucleotides, at least 80 nucleotides, at least 90 nucleotides, at least 100 nucleotides, at least 200 nucleotides, at least 300 nucleotides, at least 400 nucleotides, at least 500 nucleotides, at least 600 nucleotides, at least 700 nucleotides, at least 800 nucleotides, at least 900 nucleotides, at least 1000 nucleotides, or more) (SEQ ID NO: 93); a Kozak sequence; a 3′ untranslated region (3′ UTR); a 5′ untranslated region (5′ UTR); one or more intronic nucleotide sequences capable of being excised from the nucleic acid, or any combination thereof.

The modified mRNAs for use in the compositions, methods, and kits described herein can further comprise an internal ribosome entry site (IRES). An IRES can act as the sole ribosome binding site, or can serve as one of multiple ribosome binding sites of an mRNA. An mRNA containing more than one functional ribosome binding site can encode several peptides or polypeptides, such as the HSC inducing factors described herein, that are translated independently by the ribosomes (“multicistronic mRNA”). When nucleic acids are provided with an IRES, further optionally provided is a second translatable region. Examples of IRES sequences that can be used according to the invention include without limitation, those from picornaviruses (e.g. FMDV), pest viruses (CFFV), polio viruses (PV), encephalomyocarditis viruses (ECMV), foot-and-mouth disease viruses (FMDV), hepatitis C viruses (HCV), classical swine fever viruses (CSFV), murine leukemia virus (MLV), simian immune deficiency viruses (SW) or cricket paralysis viruses (CrPV).

In some embodiments of the compositions, methods, and kits described herein, the synthetic, modified RNA molecule comprises at least one modified nucleoside. In some embodiments of the compositions, methods, and kits described herein, the synthetic, modified RNA molecule comprises at least two modified nucleosides.

In some embodiments of the compositions, methods, and kits described herein, the modified nucleosides are selected from the group consisting of 5-methylcytosine (5mC), N6-methyladenosine (m6A), 3,2′-O-dimethyluridine (m4U), 2-thiouridine (s2U), 2′ fluorouridine, pseudouridine, 2′-O-methyluridine (Um), 2′deoxy uridine (2′ dU), 4-thiouridine (s4U), 5-methyluridine (m5U), 2′-O-methyladenosine (m6A), N6,2′-O-dimethyladenosine (m6Am), N6,N6,2′-O-trimethyladenosine (m62Am), 2′-O-methylcytidine (Cm), 7-methylguanosine (m7G), 2′-O-methylguanosine (Gm), N2,7-dimethylguanosine (m2,7G), N2,N2,7-trimethylguanosine (m2,2,7G), and inosine (I). In some embodiments, the modified nucleosides are 5-methylcytosine (5mC), pseudouracil, or a combination thereof.

Modified mRNAs need not be uniformly modified along the entire length of the molecule. Different nucleotide modifications and/or backbone structures can exist at various positions in the nucleic acid. One of ordinary skill in the art will appreciate that the nucleotide analogs or other modification(s) can be located at any position(s) of a nucleic acid such that the function of the nucleic acid is not substantially decreased. A modification can also be a 5′ or 3′ terminal modification. The nucleic acids can contain at a minimum one and at maximum 100% modified nucleotides, or any intervening percentage, such as at least 50% modified nucleotides, at least 80% modified nucleotides, or at least 90% modified nucleotides.

In some embodiments, it is preferred, but not absolutely necessary, that each occurrence of a given nucleoside in a molecule is modified (e.g., each cytosine is a modified cytosine e.g., 5-methylcytosine, each uracil is a modified uracil, e.g., pseudouracil, etc.). For example, the modified mRNAs can comprise a modified pyrimidine such as uracil or cytosine. In some embodiments, at least 25%, at least 50%, at least 80%, at least 90% or 100% of the uracil in the nucleic acid are replaced with a modified uracil. It is also contemplated that different occurrences of the same nucleoside can be modified in a different way in a given synthetic, modified RNA molecule. The modified uracil can be replaced by a compound having a single unique structure, or can be replaced by a plurality of compounds having different structures (e.g., 2, 3, 4 or more unique structures). In some embodiments, at least 25%, at least 50%, at least 80%, at least 90% or 100% of the cytosine in the nucleic acid may be replaced with a modified cytosine. The modified cytosine can be replaced by a compound having a single unique structure, or can be replaced by a plurality of compounds having different structures (e.g., 2, 3, 4 or more unique structures) (e.g., some cytosines modified as 5mC, others modified as 2′-O-methylcytosine or other cytosine analog). Such multi-modified synthetic RNA molecules can be produced by using a ribonucleoside blend or mixture comprising all the desired modified nucleosides, such that when the RNA molecules are being synthesized, only the desired modified nucleosides are incorporated into the resulting RNA molecule encoding the HSC inducing factor.

As used herein, “unmodified” or “natural” nucleosides or nucleobases include the purine bases adenine (A) and guanine (G), and the pyrimidine bases thymine (T), cytosine (C) and uracil (U). Modified nucleosides include other synthetic and natural nucleobases such as inosine, xanthine, hypoxanthine, nubularine, isoguanisine, tubercidine, 2-(halo)adenine, 2-(alkyl)adenine, 2-(propyl)adenine, 2 (amino)adenine, 2-(aminoalkyll)adenine, 2 (aminopropyl)adenine, 2 (methylthio) N6 (isopentenyl)adenine, 6 (alkyl)adenine, 6 (methyl)adenine, 7 (deaza)adenine, 8 (alkenyl)adenine, 8-(alkyl)adenine, 8 (alkynyl)adenine, 8 (amino)adenine, 8-(halo)adenine, 8-(hydroxyl)adenine, 8 (thioalkyl)adenine, 8-(thiol)adenine, N6-(isopentyl)adenine, N6 (methyl)adenine, N6,N6 (dimethyl)adenine, 2-(alkyl)guanine, 2 (propyl)guanine, 6-(alkyl)guanine, 6 (methyl)guanine, 7 (alkyl)guanine, 7 (methyl)guanine, 7 (deaza)guanine, 8 (alkyl)guanine, 8-(alkenyl)guanine, 8 (alkynyl)guanine, 8-(amino)guanine, 8 (halo)guanine, 8-(hydroxyl)guanine, 8 (thioalkyl)guanine, 8-(thiol)guanine, N(methyl)guanine, 2-(thio)cytosine, 3 (deaza) 5 (aza)cytosine, 3-(alkyl)cytosine, 3 (methyl)cytosine, 5-(alkyl)cytosine, 5-(alkynyl)cytosine, 5 (halo)cytosine, 5 (methyl)cytosine, 5 (propynyl)cytosine, 5 (propynyl)cytosine, 5 (trifluoromethyl)cytosine, 6-(azo)cytosine, N4 (acetyl)cytosine, 3 (3 amino-3 carboxypropyl)uracil, 2-(thio)uracil, 5 (methyl) 2 (thio)uracil, 5 (methylaminomethyl)-2 (thio)uracil, 4-(thio)uracil, 5 (methyl) 4 (thio)uracil, 5 (methylaminomethyl)-4 (thio)uracil, 5 (methyl) 2,4 (dithio)uracil, 5 (methylaminomethyl)-2,4 (dithio)uracil, 5 (2-aminopropyl)uracil, 5-(alkyl)uracil, 5-(alkynyl)uracil, 5-(allylamino)uracil, 5 (aminoallyl)uracil, 5 (aminoalkyl)uracil, 5 (guanidiniumalkyl)uracil, 5 (1,3-diazole-1-alkyl)uracil, 5-(cyanoalkyl)uracil, 5-(dialkylaminoalkyl)uracil, 5 (dimethylaminoalkyl)uracil, 5-(halo)uracil, 5-(methoxy)uracil, uracil-5 oxyacetic acid, 5 (methoxycarbonylmethyl)-2-(thio)uracil, 5 (methoxycarbonyl-methyl)uracil, 5 (propynyl)uracil, 5 (propynyl)uracil, 5 (trifluoromethyl)uracil, 6 (azo)uracil, dihydrouracil, N3 (methyl)uracil, 5-uracil (i.e., pseudouracil), 2 (thio)pseudouracil, 4 (thio)pseudouracil, 2,4-(dithio)psuedouracil, 5-(alkyl)pseudouracil, 5-(methyl)pseudouracil, 5-(alkyl)-2-(thio)pseudouracil, 5-(methyl)-2-(thio)pseudouracil, 5-(alkyl)-4 (thio)pseudouracil, 5-(methyl)-4 (thio)pseudouracil, 5-(alkyl)-2,4 (dithio)pseudouracil, 5-(methyl)-2,4 (dithio)pseudouracil, 1 substituted pseudouracil, 1 substituted 2(thio)-pseudouracil, 1 substituted 4 (thio)pseudouracil, 1 substituted 2,4-(dithio)pseudouracil, 1 (aminocarbonylethylenyl)-pseudouracil, 1 (aminocarbonylethylenyl)-2(thio)-pseudouracil, 1 (aminocarbonylethylenyl)-4 (thio)pseudouracil, 1 (aminocarbonylethylenyl)-2,4-(dithio)pseudouracil, 1 (aminoalkylaminocarbonylethylenyl)-pseudouracil, 1 (aminoalkylaminocarbonylethylenyl)-2(thio)-pseudouracil, 1 (aminoalkylaminocarbonylethylenyl)-4 (thio)pseudouracil, 1 (aminoalkylaminocarbonylethylenyl)-2,4-(dithio)pseudouracil, 1,3-(diaza)-2-(oxo)-phenoxazin-1-yl, 1-(aza)-2-(thio)-3-(aza)-phenoxazin-1-yl, 1,3-(diaza)-2-(oxo)-phenthiazin-1-yl, 1-(aza)-2-(thio)-3-(aza)-phenthiazin-1-yl, 7-substituted 1,3-(diaza)-2-(oxo)-phenoxazin-1-yl, 7-substituted 1-(aza)-2-(thio)-3-(aza)-phenoxazin-1-yl, 7-substituted 1,3-(diaza)-2-(oxo)-phenthiazin-1-yl, 7-substituted 1-(aza)-2-(thio)-3-(aza)-phenthiazin-1-yl, 7-(aminoalkylhydroxy)-1,3-(diaza)-2-(oxo)-phenoxazin-1-yl, 7-(aminoalkylhydroxy)-1-(aza)-2-(thio)-3-(aza)-phenoxazin-1-yl, 7-(aminoalkylhydroxy)-1,3-(diaza)-2-(oxo)-phenthiazin-1-yl, 7-(aminoalkylhydroxy)-1-(aza)-2-(thio)-3-(aza)-phenthiazin-1-yl, 7-(guanidiniumalkylhydroxy)-1,3-(diaza)-2-(oxo)-phenoxazin-1-yl, 7-(guanidiniumalkylhydroxy)-1-(aza)-2-(thio)-3-(aza)-phenoxazin-1-yl, 7-(guanidiniumalkyl-hydroxy)-1,3-(diaza)-2-(oxo)-phenthiazin-1-yl, 7-(guanidiniumalkylhydroxy)-1-(aza)-2-(thio)-3-(aza)-phenthiazin-1-yl, 1,3,5-(triaza)-2,6-(dioxa)-naphthalene, inosine, xanthine, hypoxanthine, nubularine, tubercidine, isoguanisine, inosinyl, 2-aza-inosinyl, 7-deaza-inosinyl, nitroimidazolyl, nitropyrazolyl, nitrobenzimidazolyl, nitroindazolyl, aminoindolyl, pyrrolopyrimidinyl, 3-(methyl)isocarbostyrilyl, 5-(methyl)isocarbostyrilyl, 3-(methyl)-7-(propynyl)isocarbostyrilyl, 7-(aza)indolyl, 6-(methyl)-7-(aza)indolyl, imidizopyridinyl, 9-(methyl)-imidizopyridinyl, pyrrolopyrizinyl, isocarbostyrilyl, 7-(propynyl)isocarbostyrilyl, propynyl-7-(aza)indolyl, 2,4,5-(trimethyl)phenyl, 4-(methyl)indolyl, 4,6-(dimethyl)indolyl, phenyl, napthalenyl, anthracenyl, phenanthracenyl, pyrenyl, stilbenyl, tetracenyl, pentacenyl, difluorotolyl, 4-(fluoro)-6-(methyl)benzimidazole, 4-(methyl)benzimidazole, 6-(azo)thymine, 2-pyridinone, 5 nitroindole, 3 nitropyrrole, 6-(aza)pyrimidine, 2 (amino)purine, 2,6-(diamino)purine, 5 substituted pyrimidines, N2-substituted purines, N6-substituted purines, 06-substituted purines, substituted 1,2,4-triazoles, pyrrolo-pyrimidin-2-on-3-yl, 6-phenyl-pyrrolo-pyrimidin-2-on-3-yl, para-substituted-6-phenyl-pyrrolo-pyrimidin-2-on-3-yl, ortho-substituted-6-phenyl-pyrrolo-pyrimidin-2-on-3-yl, bis-ortho-substituted-6-phenyl-pyrrolo-pyrimidin-2-on-3-yl, para-(aminoalkylhydroxy)-6-phenyl-pyrrolo-pyrimidin-2-on-3-yl, ortho-(aminoalkylhydroxy)-6-phenyl-pyrrolo-pyrimidin-2-on-3-yl, bis-ortho-(aminoalkylhydroxy)-6-phenyl-pyrrolo-pyrimidin-2-on-3-yl, pyridopyrimidin-3-yl, 2-oxo-7-amino-pyridopyrimidin-3-yl, 2-oxo-pyridopyrimidine-3-yl, or any O-alkylated or N-alkylated derivatives thereof.

In some embodiments of the compositions, methods, and kits described herein, modified nucleosides include 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine.

In other embodiments of the compositions, methods, and kits described herein, modified nucleosides include 2-aminopurine, 2,6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine.

In other embodiments of the compositions, methods, and kits described herein, modified nucleosides include inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, l-methyl-6-thio-guanosine, N2-methyl-6-thio-guanosine, and N2,N2-dimethyl-6-thio-guanosine.

In certain embodiments it is desirable to intracellularly degrade a modified nucleic acid introduced into the cell, for example if precise timing of protein production is desired. Thus, in some embodiments of the compositions, methods, and kits described herein, provided herein are modified nucleic acids comprising a degradation domain, which is capable of being acted on in a directed manner within a cell.

Modified nucleosides also include natural bases that comprise conjugated moieties, e.g. a ligand. As discussed herein above, the RNA containing the modified nucleosides must be translatable in a host cell (i.e., does not prevent translation of the polypeptide encoded by the modified RNA). For example, transcripts containing s2U and m6A are translated poorly in rabbit reticulocyte lysates, while pseudouridine, m5U, and m5C are compatible with efficient translation. In addition, it is known in the art that 2′-fluoro-modified bases useful for increasing nuclease resistance of a transcript, leads to very inefficient translation. Translation can be assayed by one of ordinary skill in the art using e.g., a rabbit reticulocyte lysate translation assay.

Accordingly, provided herein, in some aspects are hematopoietic stem cell (HSC) inducing composition comprising modified mRNA sequences encoding at least one, two, three, four, five, six, seve, eight or more HSC inducing factors selected from: CDKN1C, DNMT3B, EGR1, ETV6, EVI1, GATA2, GFI1B, GLIS2, HLF, HMGA2, HOXA5, HOXA9, HOXB3, HOXB4, HOXB5, IGF2BP2, IKZF2, KLF12, KLF4, KLF9, LMO2, MEIS1, MSI2, MYCN, NAP1L3, NDN, NFIX, NKX2-3, NR3C2, PBX1, PRDM16, PRDM5, RARB, RBBP6, RBPMS, RUNX1, RUNX1T1, SMAD6, TAL1, TCF15, VDR, ZFP37, ZFP467, ZFP521, ZFP532, and ZFP612, wherein each cytosine of each of the modified mRNA sequences is a modified cytosine, each uracil of each of the modified mRNA sequences is a modified uracil, or a combination thereof.

In some embodiments of these aspects and all such aspects described herein, the at least one, two, three, four, or more HSC inducing factors are HLF, RUNX1T1, PBX1, LMO2, PRDM5, ZFP37, MYCN, MSI2, NKX2-3, MEIS1, and RBPMS.

In some embodiments of these aspects and all such aspects described herein, the at least one, two, three, four, or more HSC inducing factors are HLF, RUNX1T1, ZFP37, PBX1, LMO2, and PRDM5

Also provided herein, in some aspects, are hematopoietic stem cell (HSC) inducing compositions comprising: a modified mRNA sequence encoding HLF; a modified mRNA sequence encoding RUNX1T1; a modified mRNA sequence encoding ZFP37; a modified mRNA sequence encoding PBX1; a modified mRNA sequence encoding LMO2; and a modified mRNA sequence encoding PRDM5; wherein each cytosine of each of the modified mRNA sequences is a modified cytosine, each uracil of each of the modified mRNA sequences is a modified uracil, or a combination thereof.

In some embodiments of these aspects and all such aspects described herein, the HSC inducing composition further comprises one or more of: a modified mRNA sequence encoding PRDM16; a modified mRNA sequence encoding ZFP467; and a modified mRNA sequence encoding VDR; wherein each cytosine of each of the modified mRNA sequences is a modified cytosine, each uracil of each of the modified mRNA sequences is a modified uracil, or a combination thereof.

Provided herein, in some aspects, are hematopoietic stem cell (HSC) inducing compositions comprising: a modified mRNA sequence encoding HLF; a modified mRNA sequence encoding RUNX1T1; a modified mRNA sequence encoding PBX1; a modified mRNA sequence encoding LMO2; a modified mRNA sequence encoding PRDM5; a modified mRNA sequence encoding ZFP37; a modified mRNA sequence encoding MYCN; a modified mRNA sequence encoding MSI2; a modified mRNA sequence encoding NKX2-3; a modified mRNA sequence encoding MEIS1; and a modified mRNA sequence encoding RBPMS; wherein each cytosine of each of the modified mRNA sequences is a modified cytosine, each uracil of each of the modified mRNA sequences is a modified uracil, or a combination thereof.

Also provided herein are hematopoietic stem cell (HSC) inducing compositions comprising: a modified mRNA sequence encoding ZFP467; a modified mRNA sequence encoding PBX1; a modified mRNA sequence encoding HOXB4; and a modified mRNA sequence encoding MSI2; wherein each cytosine of each of the modified mRNA sequences is a modified cytosine, each uracil of each of the modified mRNA sequences is a modified uracil, or a combination thereof.

In some embodiments of these aspects and all such aspects described herein, the HSC inducing composition further comprises one or more of: a modified mRNA sequence encoding HLF; a modified mRNA sequence encoding LMO2; a modified mRNA sequence encoding PRDM16; and a modified mRNA sequence encoding ZFP37, wherein each cytosine of each of the modified mRNA sequences is a modified cytosine, each uracil of each of the modified mRNA sequences is a modified uracil, or a combination thereof.

Provided herein, in some aspects, are hematopoietic stem cell (HSC) inducing compositions comprising: a modified mRNA sequence encoding MYCN; a modified mRNA sequence encoding MSI2; a modified mRNA sequence encoding NKX2-3; and a modified mRNA sequence encoding RUNX1T1; wherein each cytosine of each of the modified mRNA sequences is a modified cytosine, each uracil of each of the modified mRNA sequences is a modified uracil, or a combination thereof.

In some embodiments of these aspects and all such aspects described herein, the HSC inducing composition further comprises one or more of: a modified mRNA sequence encoding HOXB5; a modified mRNA sequence encoding HLF; a modified mRNA sequence encoding ZFP467; a modified mRNA sequence encoding HOXB3; a modified mRNA sequence encoding LMO2; a modified mRNA sequence encoding PBX1; a modified mRNA sequence encoding ZFP37; and a modified mRNA sequence encoding ZFP521; wherein each cytosine of each of the modified mRNA sequences is a modified cytosine, each uracil of each of the modified mRNA sequences is a modified uracil, or a combination thereof.

Provided herein, in some aspects, are hematopoietic stem cell (HSC) inducing compositions comprising: a modified mRNA sequence encoding HOXB4; a modified mRNA sequence encoding PBX1; a modified mRNA sequence encoding LMO2; a modified mRNA sequence encoding ZFP467; and a modified mRNA sequence encoding ZFP521; wherein each cytosine of each of the modified mRNA sequences is a modified cytosine, each uracil of each of the modified mRNA sequences is a modified uracil, or a combination thereof.

In some embodiments of these aspects and all such aspects described herein, the HSC inducing composition further comprises one or more of: a modified mRNA sequence encoding KLF12; a modified mRNA sequence encoding HLF; and a modified mRNA sequence encoding EGR; wherein each cytosine of each of the modified mRNA sequences is a modified cytosine, each uracil of each of the modified mRNA sequences is a modified uracil, or a combination thereof.

Also provided herein are hematopoietic stem cell (HSC) inducing compositions comprising: a modified mRNA sequence encoding MEIS1; a modified mRNA sequence encoding RBPMS; a modified mRNA sequence encoding ZFP37; a modified mRNA sequence encoding RUNX1T1; and a modified mRNA sequence encoding LMO2; wherein each cytosine of each of the modified mRNA sequences is a modified cytosine, each uracil of each of the modified mRNA sequences is a modified uracil, or a combination thereof.

In some embodiments of these aspects and all such aspects described herein, the HSC inducing composition further comprises one or more of: a modified mRNA sequence encoding KLF12; and a modified mRNA sequence encoding HLF; wherein each cytosine of each of the modified mRNA sequences is a modified cytosine, each uracil of each of the modified mRNA sequences is a modified uracil, or a combination thereof.

Also provided herein are hematopoietic stem cell (HSC) inducing compositions comprising: a modified mRNA sequence encoding ZFP37; a modified mRNA sequence encoding HOXB4; a modified mRNA sequence encoding LMO2; and a modified mRNA sequence encoding HLF; wherein each cytosine of each of the modified mRNA sequences is a modified cytosine, each uracil of each of the modified mRNA sequences is a modified uracil, or a combination thereof.

In some embodiments of these aspects and all such aspects described herein, the HSC inducing composition further comprises one or more of: a modified mRNA encoding MYCN; a modified mRNA encoding ZFP467; a modified mRNA encoding NKX2-3; a modified mRNA encoding PBX1; and a modified mRNA encoding KLF4; wherein each cytosine of each of the modified mRNA sequences is a modified cytosine, each uracil of each of the modified mRNA sequences is a modified uracil, or a combination thereof.

In some embodiments of these aspects and all such aspects described herein, the modified cytosine is 5-methylcytosine and the modified uracil is pseudouridine.

The modified mRNAs encoding HSC inducing factors described herein can be synthesized and/or modified by methods well established in the art, such as those described in “Current Protocols in Nucleic Acid Chemistry,” Beaucage, S. L. et al. (Edrs.), John Wiley & Sons, Inc., New York, N.Y., USA, which is hereby incorporated herein by reference in its entirety. In some embodiments of the compositions, methods, and kits described herein, the modified mRNAs encoding the HSC inducing factor(s), such as HLF, RUNX1T1, PBX1, LMO2, PRDM5, ZFP37, MYCN, MSI2, NKX2-3, MEIS1, and RBPMS, are generated using the IVT templates and constructs, and methods thereof for rapidly and efficiently generating synthetic RNAs described in PCT Application No.: PCT/US12/64359, filed Nov. 9, 2012, and as described in US 20120251618 A1, the contents of each of which are herein incorporated by reference in their entireties. In some embodiments of the compositions, methods, and kits described herein, the synthetic, modified RNAs encoding the HSC inducing factor(s), such as HLF, RUNX1T1, PBX1, LMO2, PRDM5, ZFP37, MYCN, MSI2, NKX2-3, MEIS1, and RBPMS, are delivered and formulated as described in US 20120251618 A1.

One of skill in the art can easily monitor the expression level of the polypeptide encoded by a synthetic, modified RNA using e.g., Western blotting techniques or immunocytochemistry techniques. A synthetic, modified RNA can be administered at a frequency and dose that permit a desired level of expression of the polypeptide. Each different modified mRNA can be administered at its own dose and frequency to permit appropriate expression. In addition, since the modified RNAs administered to the cell are transient in nature (i.e., are degraded over time) one of skill in the art can easily remove or stop expression of a modified RNA by halting further transfections and permitting the cell to degrade the modified RNA over time. The modified RNAs will degrade in a manner similar to cellular mRNAs.

Accordingly, in some embodiments of the compositions, methods, and kits described herein, a plurality of synthetic, modified RNAs encoding HSC inducing factors can be contacted with, or introduced to, a cell, population of cells, or cell culture simultaneously. In other embodiments, the plurality of synthetic, modified RNAs encoding HSC inducing factors can be contacted with, or introduced to, a cell, population of cells, or cell culture separately. In addition, each modified RNA encoding an HSC inducing factor can be administered according to its own dosage regime.

In some embodiments of the compositions, methods, and kits described herein, a modified RNA encoding an HSC inducing factor can be introduced into target cells by transfection or lipofection. Suitable agents for transfection or lipofection include, for example, calcium phosphate, DEAE dextran, lipofectin, lipofectamine, DIMRIE C™, Superfect™, and Effectin™ (Qiagen™) Unifectin™, Maxifectin™, DOTMA, DOGS™ (Transfectam; dioctadecylamidoglycylspermine), DOPE (1,2-dioleoyl-sn-glycero-3-phosphoethanolamine), DOTAP (1,2-dioleoyl-3-trimethylammonium propane), DDAB (dimethyl dioctadecylammonium bromide), DHDEAB (N,N-di-n-hexadecyl-N,N-dihydroxyethyl ammonium bromide), HDEAB (N-n-hexadecyl-N,N-dihydroxyethylammonium bromide), polybrene, poly(ethylenimine) (PEI), and the like. (See, e.g., Banerjee et al., Med. Chem. 42:4292-99 (1999); Godbey et al., Gene Ther. 6:1380-88 (1999); Kichler et al., Gene Ther. 5:855-60 (1998); Birchaa et al., J. Pharm. 183:195-207 (1999)).

In some embodiments, a modified RNA can be transfected into target cells as a complex with cationic lipid carriers (e.g., OLIGOFECTAMINE™) or non-cationic lipid-based carriers (e.g., Transit-TKOTM™, Mirus Bio LLC, Madison, Wis.).

In some embodiments of the aspects described herein, the synthetic, modified RNA is introduced into a cell using a transfection reagent. Some exemplary transfection reagents include, for example, cationic lipids, such as lipofectin (Junichi et al, U.S. Pat. No. 5,705,188), cationic glycerol derivatives, and polycationic molecules, such as polylysine (Lollo et al., PCT Application WO 97/30731). Examples of commercially available transfection reagents are known to those of ordinary skill in the art.

In other embodiments, highly branched organic compounds, termed “dendrimers,” can be used to bind the exogenous nucleic acid, such as the synthetic, modified RNAs described herein, and introduce it into the cell.

In other embodiments of the aspects described herein, non-chemical methods of transfection are contemplated. Such methods include, but are not limited to, electroporation, sonoporation, the use of a gene gun, magnetofection, and impalefection, and others, as known to those of ordinary skill in the art. Other agents may be utilized to enhance the penetration of the administered nucleic acids, including glycols, such as ethylene glycol and propylene glycol, pyrrols such as 2-pyrrol, azones, and terpenes, such as limonene and menthone.

In some embodiments of the compositions, methods, and kits described herein, a modified RNA encoding an HSC inducing factor is formulated in conjunction with one or more penetration enhancers, surfactants and/or chelators. Suitable surfactants include fatty acids and/or esters or salts thereof, bile acids and/or salts thereof. In some embodiments, combinations of penetration enhancers are used, for example, fatty acids/salts in combination with bile acids/salts. One exemplary combination is the sodium salt of lauric acid, capric acid and UDCA. Further penetration enhancers include polyoxyethylene-9-lauryl ether, polyoxyethylene-20-cetyl ether.

In some embodiments of the compositions, methods, and kits described herein, a modified RNA encoding an HSC inducing factor is formulated into any of many possible administration forms, including a sustained release form. In some embodiments of the compositions, methods, and kits described herein, formulations comprising a plurality of different synthetic, modified RNAs encoding HSC inducing factors are prepared by first mixing all members of a plurality of different synthetic, modified RNAs, and then complexing the mixture comprising the plurality of different synthetic, modified RNAs with a desired ligand or targeting moiety, such as a lipid. The compositions can be formulated as suspensions in aqueous, non-aqueous or mixed media. Aqueous suspensions can further contain substances which increase the viscosity of the suspension including, for example, sodium carboxymethylcellulose, sorbitol and/or dextran. The suspension can also contain stabilizers.

The compositions described herein can be prepared and formulated as emulsions for the delivery of synthetic, modified RNAs. Emulsions can contain further components in addition to the dispersed phases, and the active drug (i.e., synthetic, modified RNA) which can be present as a solution in either the aqueous phase, oily phase or itself as a separate phase. Pharmaceutical excipients such as emulsifiers, stabilizers, dyes, and anti-oxidants can also be present in emulsions as needed. Emulsions can also be multiple emulsions that are comprised of more than two phases such as, for example, in the case of oil-in-water-in-oil (o/w/o) and water-in-oil-in-water (w/o/w) emulsions. Emulsifiers can broadly be classified into four categories: synthetic surfactants, naturally occurring emulsifiers, absorption bases, and finely dispersed solids (see e.g., Ansel's Pharmaceutical Dosage Forms and Drug Delivery Systems, Allen, L V., Popovich N G., and Ansel H C., 2004, Lippincott Williams & Wilkins (8th ed.), New York, N.Y.; Idson, in Pharmaceutical Dosage Forms, Lieberman, Rieger and Banker (Eds.), 1988, Marcel Dekker, Inc., New York, N.Y., volume 1, p. 199).

In some embodiments of the compositions, methods, and kits described herein, a modified RNA encoding an HSC inducing factor can be encapsulated in a nanoparticle. Methods for nanoparticle packaging are well known in the art, and are described, for example, in Bose S, et al (Role of Nucleolin in Human Parainfluenza Virus Type 3 Infection of Human Lung Epithelial Cells. J. Virol. 78:8146. 2004); Dong Y et al. Poly(d,l-lactide-co-glycolide)/montmorillonite nanoparticles for oral delivery of anticancer drugs. Biomaterials 26:6068. 2005); Lobenberg R. et al (Improved body distribution of 14C-labelled AZT bound to nanoparticles in rats determined by radioluminography. J Drug Target 5:171.1998); Sakuma S R et al (Mucoadhesion of polystyrene nanoparticles having surface hydrophilic polymeric chains in the gastrointestinal tract. Int J Pharm 177:161. 1999); Virovic L et al. Novel delivery methods for treatment of viral hepatitis: an update. Expert Opin Drug Deliv 2:707.2005); and Zimmermann E et al, Electrolyte- and pH-stabilities of aqueous solid lipid nanoparticle (SLN) dispersions in artificial gastrointestinal media. Eur J Pharm Biopharm 52:203. 2001), the contents of which are herein incorporated in their entireties by reference.

While it is understood that iHSCs can be generated by delivery of HSC inducing factors in the form of nucleic acid (DNA or RNA) or amino acid sequences, in some embodiments of the compositions, methods, and kits described herein, iHSC induction can be induced using other methods, such as, for example, by treatment of cells with an agent, such as a small molecule or cocktail of small molecules, that induce expression one or more of the HSC inducing factors.

The term “agent” as used herein means any compound or substance such as, but not limited to, a small molecule, nucleic acid, polypeptide, peptide, drug, ion, etc. An “agent” can be any chemical, entity or moiety, including without limitation synthetic and naturally-occurring proteinaceous and non-proteinaceous entities. In some embodiments, an agent is nucleic acid, nucleic acid analogues, proteins, antibodies, peptides, aptamers, oligomer of nucleic acids, amino acids, or carbohydrates including without limitation proteins, oligonucleotides, ribozymes, DNAzymes, glycoproteins, siRNAs, lipoproteins, aptamers, and modifications and combinations thereof etc. In some embodiments, the nucleic acid is DNA or RNA, and nucleic acid analogues, for example can be PNA, pcPNA and LNA. A nucleic acid may be single or double stranded, and can be selected from a group comprising; nucleic acid encoding a protein of interest, oligonucleotides, PNA, etc. Such nucleic acid sequences include, for example, but not limited to, nucleic acid sequence encoding proteins that act as transcriptional repressors, antisense molecules, ribozymes, small inhibitory nucleic acid sequences, for example but not limited to RNAi, shRNAi, siRNA, micro RNAi (mRNAi), antisense oligonucleotides etc. A protein and/or peptide agent or fragment thereof, can be any protein of interest, for example, but not limited to; mutated proteins; therapeutic proteins; truncated proteins, wherein the protein is normally absent or expressed at lower levels in the cell. Proteins of interest can be selected from a group comprising; mutated proteins, genetically engineered proteins, peptides, synthetic peptides, recombinant proteins, chimeric proteins, antibodies, humanized proteins, humanized antibodies, chimeric antibodies, modified proteins and fragments thereof.

Also provided herein, in some aspects, are methods of making, preparing, or generating induced hematopoietic stem cells using one or more expression vectors or one or more modified mRNA sequences encoding specific combinations of the HSC inducing factors described herein, such as at least one, two, three, four, five, six, seven, eight, or more of the HSC inducing factors selected from: CDKN1C, DNMT3B, EGR1, ETV6, EVI1, GATA2, GFI1B, GLIS2, HLF, HMGA2, HOXA5, HOXA9, HOXB3, HOXB4, HOXB5, IGF2BP2, IKZF2, KLF12, KLF4, KLF9, LMO2, MEIS1, MSI2, MYCN, NAP1L3, NDN, NFIX, NKX2-3, NR3C2, PBX1, PRDM16, PRDM5, RARB, RBBP6, RBPMS, RUNX1, RUNX1T1, SMAD6, TAL1, TCF15, VDR, ZFP37, ZFP467, ZFP521, ZFP532, and ZFP612.

Accordingly, provided herein, in some aspects, are methods for preparing an induced hematopoietic stem cell (iHSC) from a somatic cell comprising:
a. transducing the somatic cell with one or more vectors comprising a nucleic acid sequence encoding HLF, a nucleic acid sequence encoding RUNX1T1; a nucleic acid sequence encoding PBX1; a nucleic acid sequence encoding ZFP37; a nucleic acid sequence encoding LMO2; and a nucleic acid sequence encoding PRDM5, wherein each said nucleic acid sequence is operably linked to a promoter; and
b. culturing the transduced somatic cell in a cell media that supports growth of hematopoietic stem cells, thereby preparing an iHSC.

In some embodiments of these methods and all such method described herein, the transducing of step (a) further comprises one or more vectors comprising one or more of: a nucleic acid sequence encoding PRDM16; a nucleic acid sequence encoding ZFP467; and a nucleic acid sequence encoding VDR.

Also provided herein, in some aspects, are methods for preparing an induced hematopoietic stem cell (iHSC) from a somatic cell comprising:
a. transducing the somatic cell with one or more vectors comprising a nucleic acid sequence encoding HLF, a nucleic acid sequence encoding RUNX1T1; a nucleic acid sequence encoding PBX1; a nucleic acid sequence encoding LMO2; a nucleic acid sequence encoding PRDM5; a nucleic acid sequence encoding ZFP37; a nucleic acid sequence encoding MYCN; a nucleic acid sequence encoding MSI2; a nucleic acid sequence encoding NKX2-3; a nucleic acid sequence encoding MEIS1; and a nucleic acid sequence encoding RBPMS; wherein each said nucleic acid sequence is operably linked to a promoter; and
b. culturing the transduced somatic cell in a cell media that supports growth of hematopoietic stem cells, thereby preparing an iHSC.

Also provided herein, in some aspects, are methods for preparing an induced hematopoietic stem cell (iHSC) from a somatic cell comprising:
a. transducing the somatic cell with one or more vectors comprising a nucleic acid sequence encoding ZFP467, a nucleic acid sequence encoding PBX1; a nucleic acid sequence encoding HOXB4; and a nucleic acid sequence encoding MSI2; wherein each said nucleic acid sequence is operably linked to a promoter; and
b. culturing the transduced somatic cell in a cell media that supports growth of hematopoietic stem cells, thereby preparing an iHSC.

In some embodiments of these methods and all such method described herein, the transducing of step (a) further comprises one or more vectors comprising one or more of: a nucleic acid sequence encoding HLF, a nucleic acid sequence encoding LMO2; a nucleic acid sequence encoding PRDM16; and a nucleic acid sequence encoding ZFP37.

Also provided herein, in some aspects, are methods for preparing an induced hematopoietic stem cell (iHSC) from a somatic cell comprising:
a. transducing the somatic cell with one or more vectors comprising a nucleic acid sequence encoding MYCN; a nucleic acid sequence encoding MSI2, a nucleic acid sequence encoding NKX2-3; and a nucleic acid sequence encoding RUNX1T1; wherein each said nucleic acid sequence is operably linked to a promoter; and
b. culturing the transduced somatic cell in a cell media that supports growth of hematopoietic stem cells, thereby preparing an iHSC.

In some embodiments of these methods and all such method described herein, the transducing of step (a) further comprises one or more vectors comprising one or more of: a nucleic acid sequence encoding HOXB5; a nucleic acid sequence encoding HLF, a nucleic acid sequence encoding ZFP467; a nucleic acid sequence encoding HOXB3; a nucleic acid sequence encoding LMO2; a nucleic acid sequence encoding PBX1; a nucleic acid sequence encoding ZFP37; and a nucleic acid sequence encoding ZFP521.

Also provided herein, in some aspects, are methods for preparing an induced hematopoietic stem cell (iHSC) from a somatic cell comprising:
a. transducing the somatic cell with one or more vectors comprising a nucleic acid sequence encoding HOXB4; a nucleic acid sequence encoding PBX1, a nucleic acid sequence encoding LMO2; a nucleic acid sequence encoding ZFP467; and a nucleic acid sequence encoding ZFP521; wherein each said nucleic acid sequence is operably linked to a promoter; and
b. culturing the transduced somatic cell in a cell media that supports growth of hematopoietic stem cells, thereby preparing an iHSC.

In some embodiments of these methods and all such method described herein, the transducing of step (a) further comprises one or more vectors comprising one or more of: a nucleic acid sequence encoding KLF12; a nucleic acid sequence encoding HLF; and a nucleic acid sequence encoding EGR1.

Also provided herein, in some aspects, are methods for preparing an induced hematopoietic stem cell (iHSC) from a somatic cell comprising:
a. transducing the somatic cell with one or more vectors comprising a nucleic acid sequence encoding MEIS1; a nucleic acid sequence encoding RBPMS; a nucleic acid sequence encoding ZFP37; a nucleic acid sequence encoding RUNX1T1; and a nucleic acid sequence encoding LMO2; wherein each said nucleic acid sequence is operably linked to a promoter; and
b. culturing the transduced somatic cell in a cell media that supports growth of hematopoietic stem cells, thereby preparing an iHSC.

In some embodiments of these methods and all such method described herein, the transducing of step (a) further comprises one or more vectors comprising one or more of: a nucleic acid sequence encoding KLF12; and a nucleic acid sequence encoding HLF.

Provided herein, in some aspects, are methods for preparing an induced hematopoietic stem cell (iHSC) from a somatic cell comprising:
a. transducing the somatic cell with one or more vectors comprising a nucleic acid sequence encoding ZFP37; a nucleic acid sequence encoding HOXB4; a nucleic acid sequence encoding LMO2; and a nucleic acid sequence encoding HLF; wherein each said nucleic acid sequence is operably linked to a promoter; and
b. culturing the transduced somatic cell in a cell media that supports growth of hematopoietic stem cells, thereby preparing an iHSC.

In some embodiments of these methods and all such method described herein, the transducing of step (a) further comprises one or more vectors comprising one or more of: a nucleic acid sequence encoding KLF12; and a nucleic acid sequence encoding HLF.

Also provided herein, in some aspects, are methods for preparing an induced hematopoietic stem cell (iHSC) from a somatic cell comprising:
a. transducing the somatic cell with one or more vectors comprising a nucleic acid sequence encoding ZFP37; a nucleic acid sequence encoding HOXB4; a nucleic acid sequence encoding LMO2; and a nucleic acid sequence encoding HLF; wherein each said nucleic acid sequence is operably linked to a promoter; and
b. culturing the transduced somatic cell in a cell media that supports growth of hematopoietic stem cells, thereby preparing an iHSC.

In some embodiments of these methods and all such method described herein, the transducing of step (a) further comprises one or more vectors comprising one or more of: a nucleic acid sequence encoding MYCN; a nucleic acid sequence encoding ZFP467; a nucleic acid sequence encoding NKX2-3; a nucleic acid sequence encoding PBX1; and a nucleic acid sequence encoding KLF.

Also provided herein, in some aspects, are methods for preparing an induced hematopoietic stem cell (iHSC) from a somatic cell comprising:
a. repeatedly transfecting a somatic cell with one or more modified mRNA sequences encoding at least one, two, three, four, five, six, seve, eight, or more HSC inducing factors selected from: CDKN1C, DNMT3B, EGR1, ETV6, EVI1, GATA2, GFI1B, GLIS2, HLF, HMGA2, HOXA5, HOXA9, HOXB3, HOXB4, HOXB5, IGF2BP2, IKZF2, KLF12, KLF4, KLF9, LMO2, MEIS1, MSI2, MYCN, NAP1L3, NDN, NFIX, NKX2-3, NR3C2, PBX1, PRDM16, PRDM5, RARB, RBBP6, RBPMS, RUNX1, RUNX1T1, SMAD6, TAL1, TCF15, VDR, ZFP37, ZFP467, ZFP521, ZFP532, and ZFP612, wherein each cytosine of each of the modified mRNA sequences is a modified cytosine, each uracil of each of the modified mRNA sequences is a modified uracil, or a combination thereof
b. culturing the transfected somatic cell in a cell media that supports growth of hematopoietic stem cells, thereby preparing an iHSC.

In some embodiments of these methods and all such methods described herein, the at least one, two, three, four, or more HSC inducing factors of step (a) are HLF, RUNX1T1, PBX1, LMO2, PRDM5, ZFP37, MYCN, MSI2, NKX2-3, MEIS1, and RBPMS.

In some embodiments of these methods and all such methods described herein, the at least one, two, three, four, or more HSC inducing factors of step (a) are HLF, RUNX1T1, ZFP37, PBX1, LMO2, and PRDM5. In some such embodiments, the at least one, two, three, four, or more HSC inducing factors of step (a) further comprise one or more of: PRDM16; ZFP467; and VDR.

In some embodiments of these methods and all such methods described herein, the at least one, two, three, four, or more HSC inducing factors of step (a) are HLF; RUNX1T1; PBX1; LMO2; PRDM5; ZFP37; MYCN; MSI2; NKX2-3; MEIS1; and RBPMS.

In some embodiments of these methods and all such methods described herein, the at least one, two, three, four, or more HSC inducing factors of step (a) are ZFP467; PBX1; HOXB4; and MSI2. In some such embodiments, the at least one, two, three, four, or more HSC inducing factors of step (a) further comprise one or more of: HLF; LMO2; PRDM16; and ZFP37.

In some embodiments of these methods and all such methods described herein, the at least one, two, three, four, or more HSC inducing factors of step (a) are MYCN; MSI2; NKX2-3; and RUNX1T1. In some such embodiments, the at least one, two, three, four, or more HSC inducing factors of step (a) further comprise one or more of: HOXB5; HLF; ZFP467; HOXB3; LMO2; PBX1; ZFP37; and ZFP521.

In some embodiments of these methods and all such methods described herein, the at least one, two, three, four, or more HSC inducing factors of step (a) are HOXB4; PBX1; LMO2; ZFP467; and ZFP521. In some such embodiments, the at least one, two, three, four, or more HSC inducing factors of step (a) further comprise one or more of: KLF12; HLF; and EGR.

In some embodiments of these methods and all such methods described herein, the at least one, two, three, four, or more HSC inducing factors of step (a) are MEIS1; RBPMS; ZFP37; RUNX1T1; and LMO2. In some such embodiments, the at least one, two, three, four, or more HSC inducing factors of step (a) further comprise one or more of: KLF12; and HLF.

In some embodiments of these methods and all such methods described herein, the at least one, two, three, four, or more HSC inducing factors of step (a) are ZFP37; HOXB4; LMO2; and HLF. In some such embodiments, the at least one, two, three, four, or more HSC inducing factors of step (a) further comprise one or more of: MYCN; ZFP467; NKX2-3; PBX1; and KLF4.

Detection of expression of HSC inducing factors introduced into cells or induced in a cell population using the compositions, methods, and kits described herein, can be achieved by any of several techniques known to those of skill in the art including, for example, Western blot analysis, immunocytochemistry, and fluorescence-mediated detection.

In order to distinguish whether a given combination of HSC inducing factors has generated iHSCs or other committed progenitors, one or more HSC activities or parameters can be measured, such as, in some embodiments, differential expression of surface antigens. The generation of induced HSCs using the compositions, methods, and kits described herein preferably causes the appearance of the cell surface phenotype characteristic of endogenous HSCs, such as lineage marker negative, Sca1-positive, cKit-positive (or LSK cells), CD34-negative, Flk2-negative, CD48-negative, and CD150-positive or as CD150+CD48−CD244−, for example.

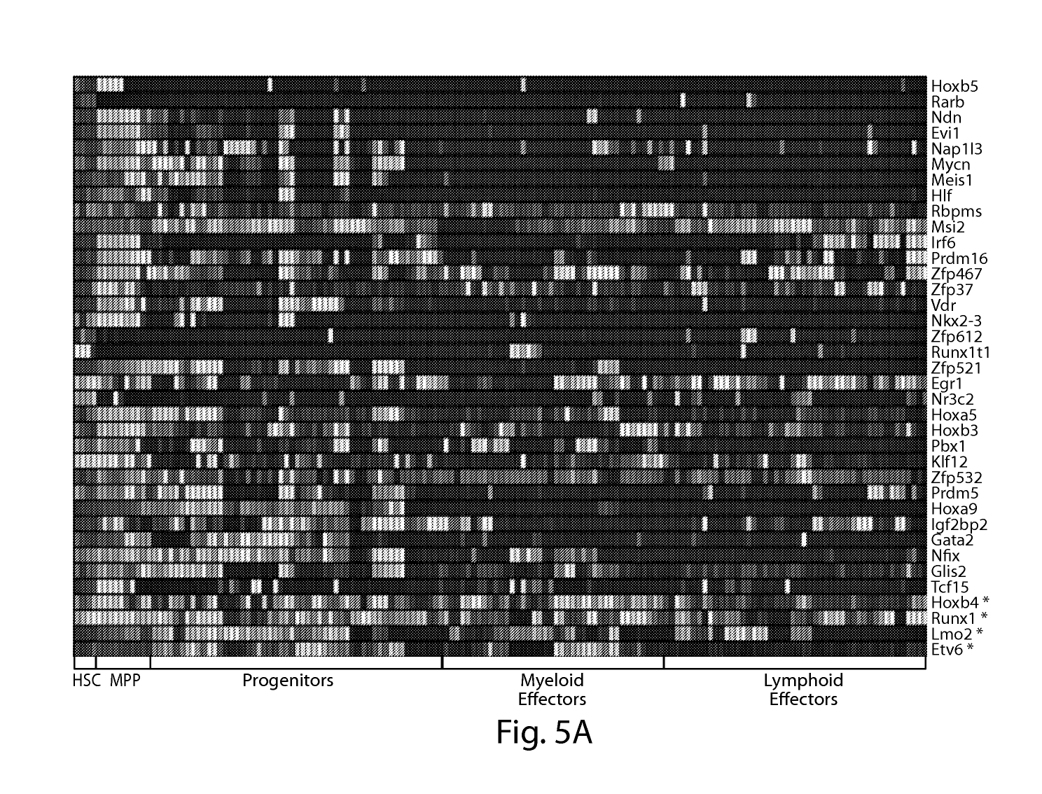

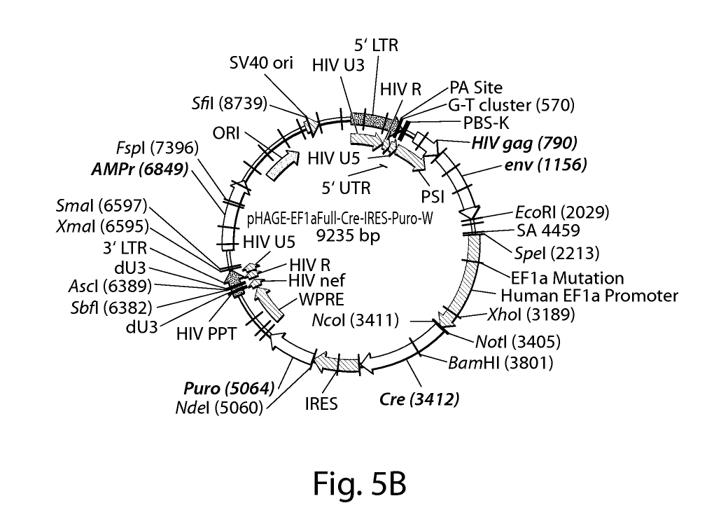

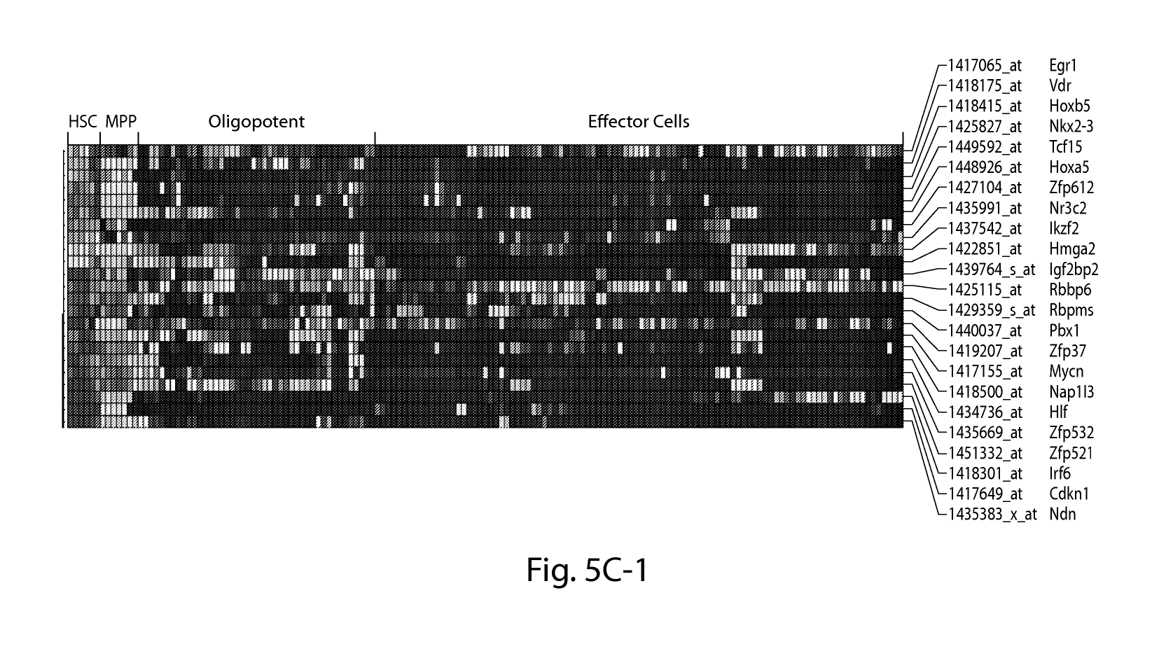

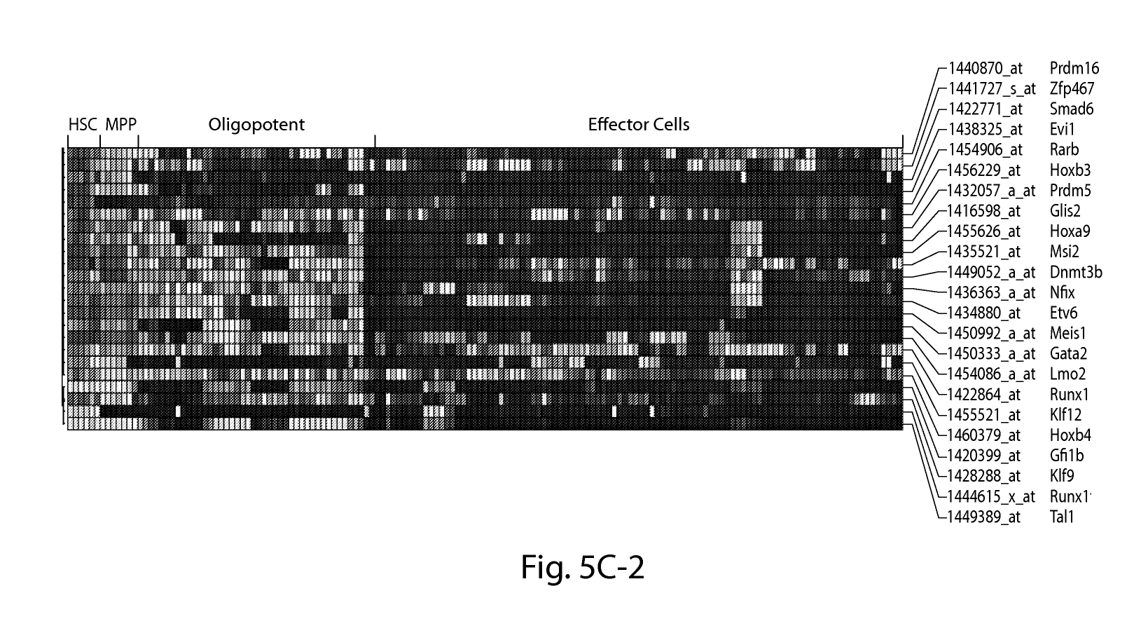

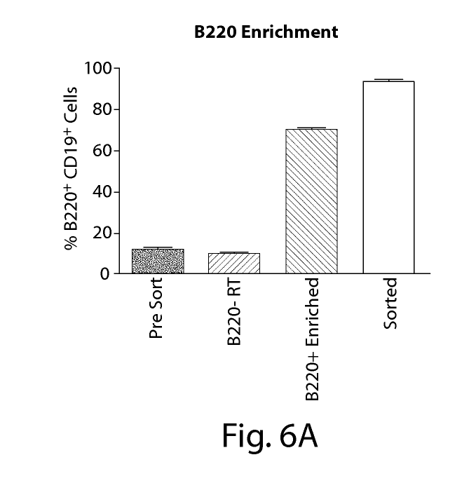

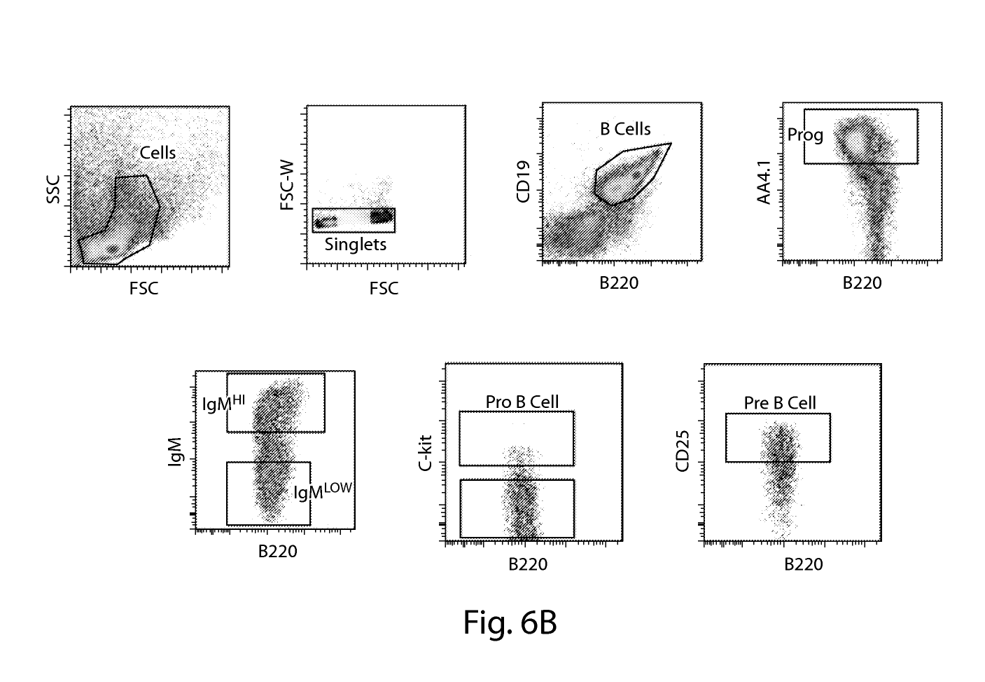

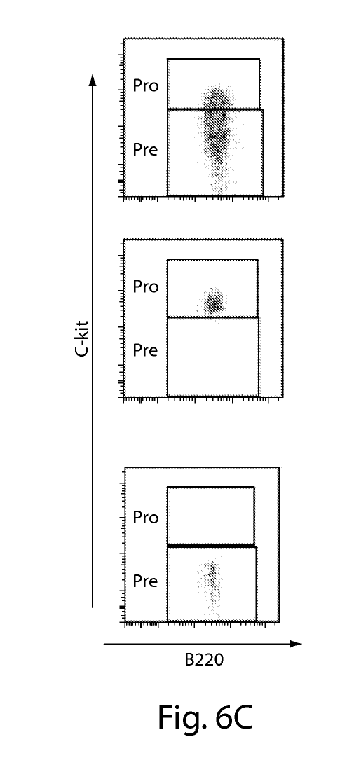

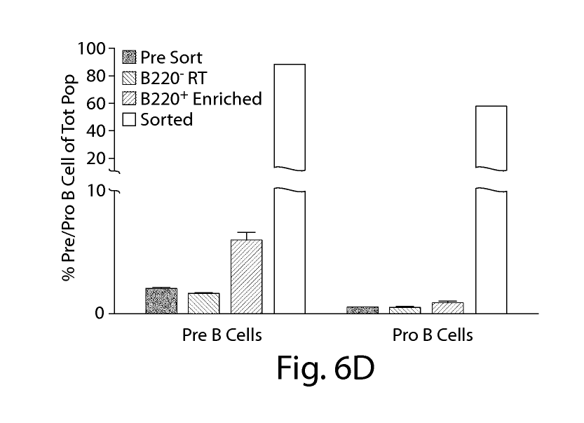

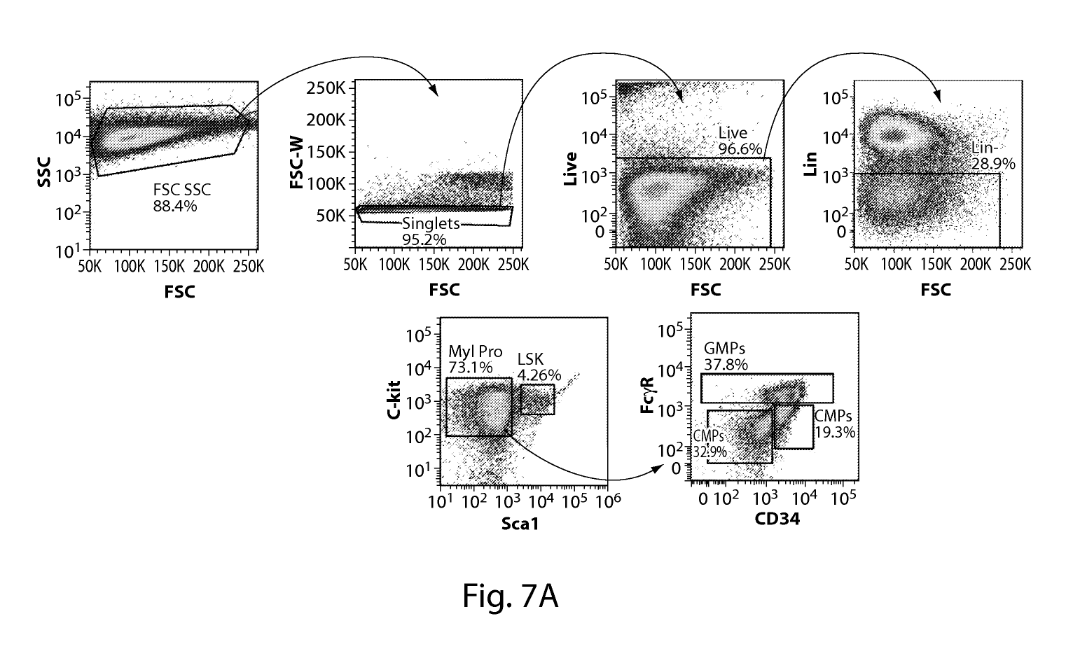

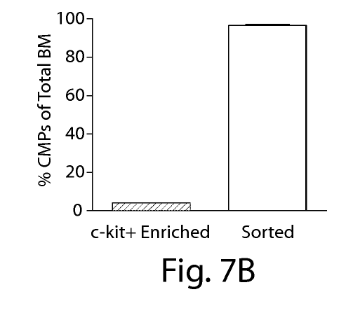

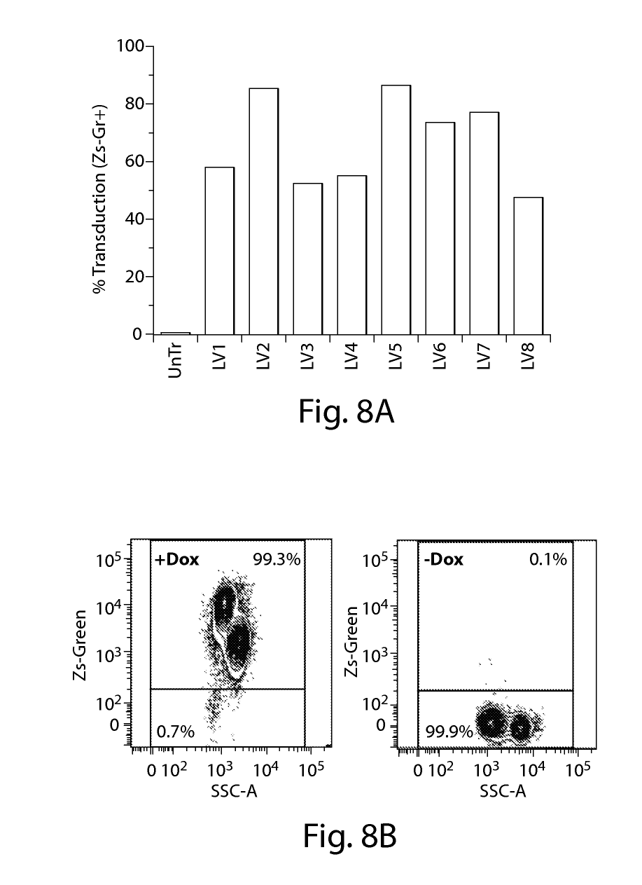

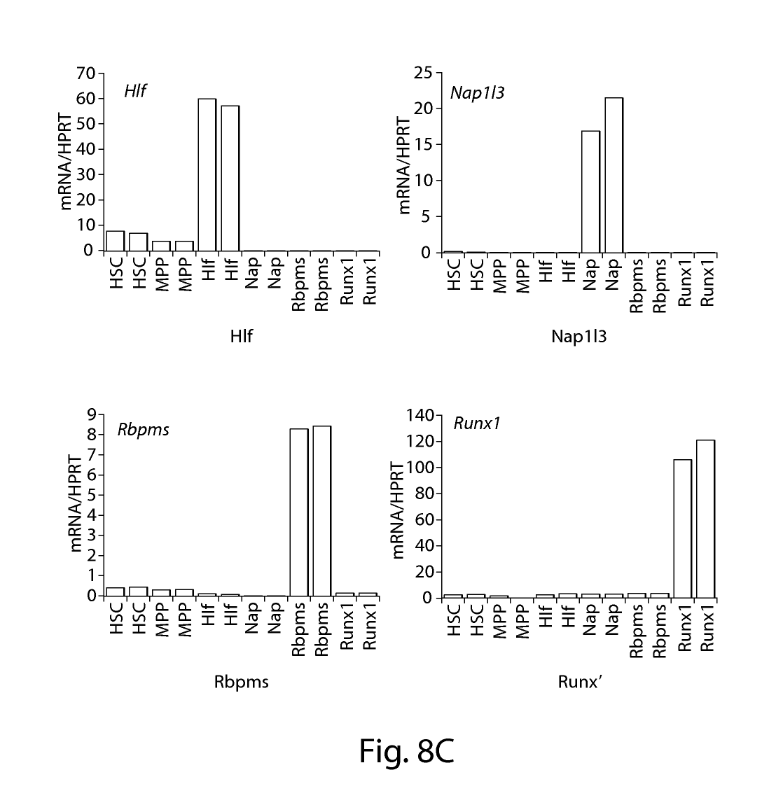

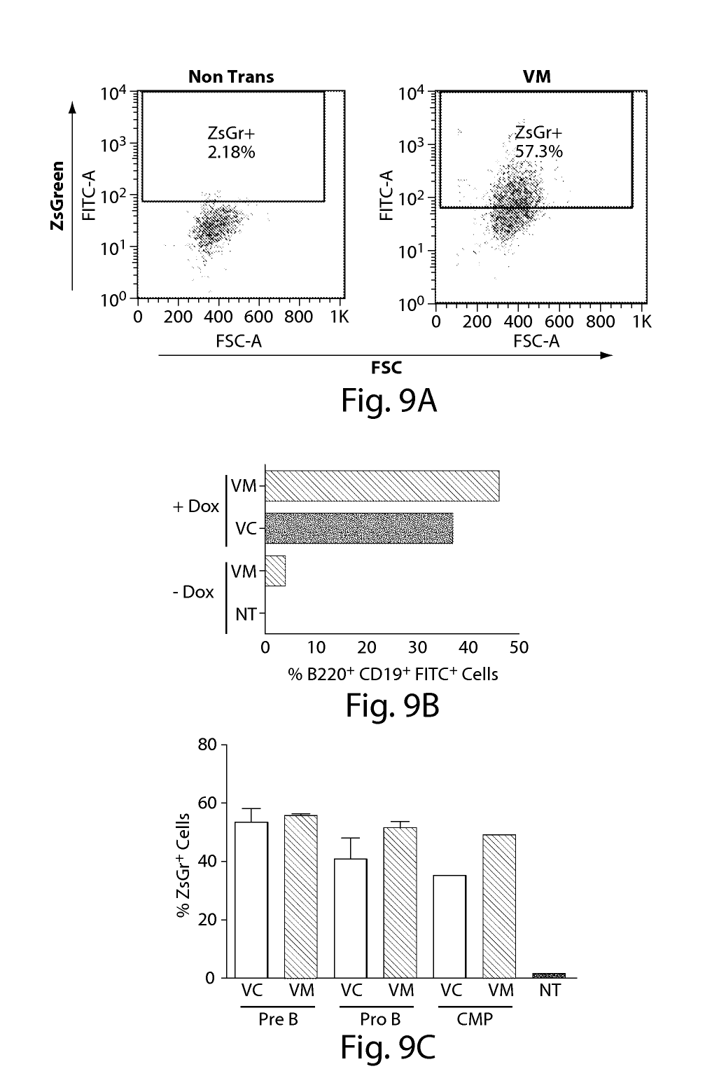

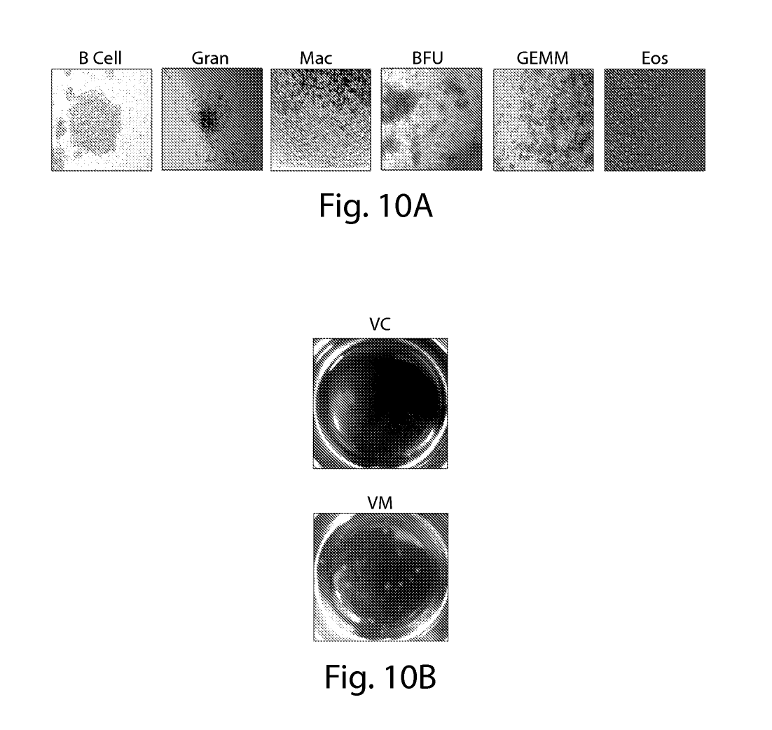

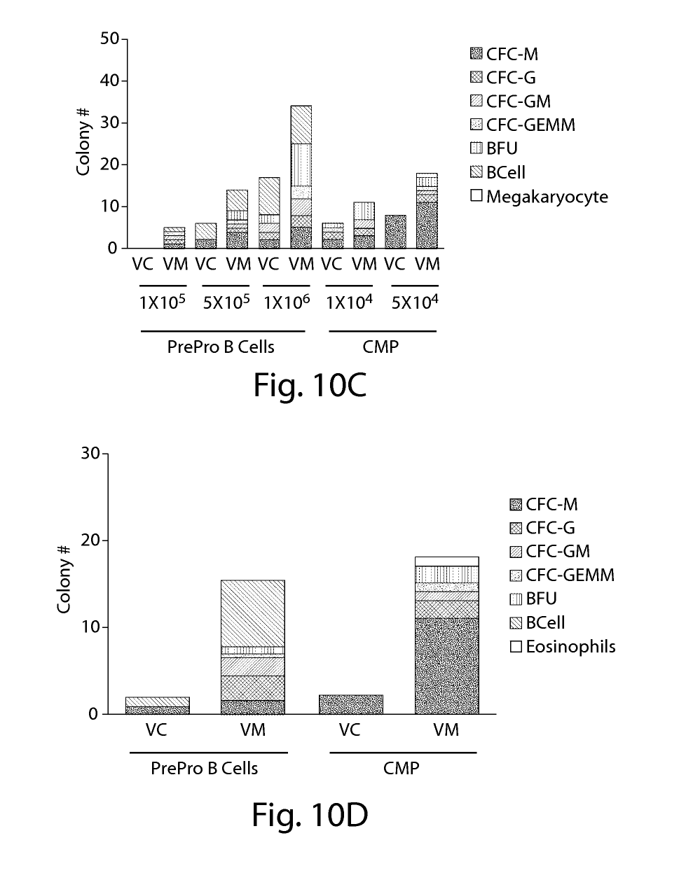

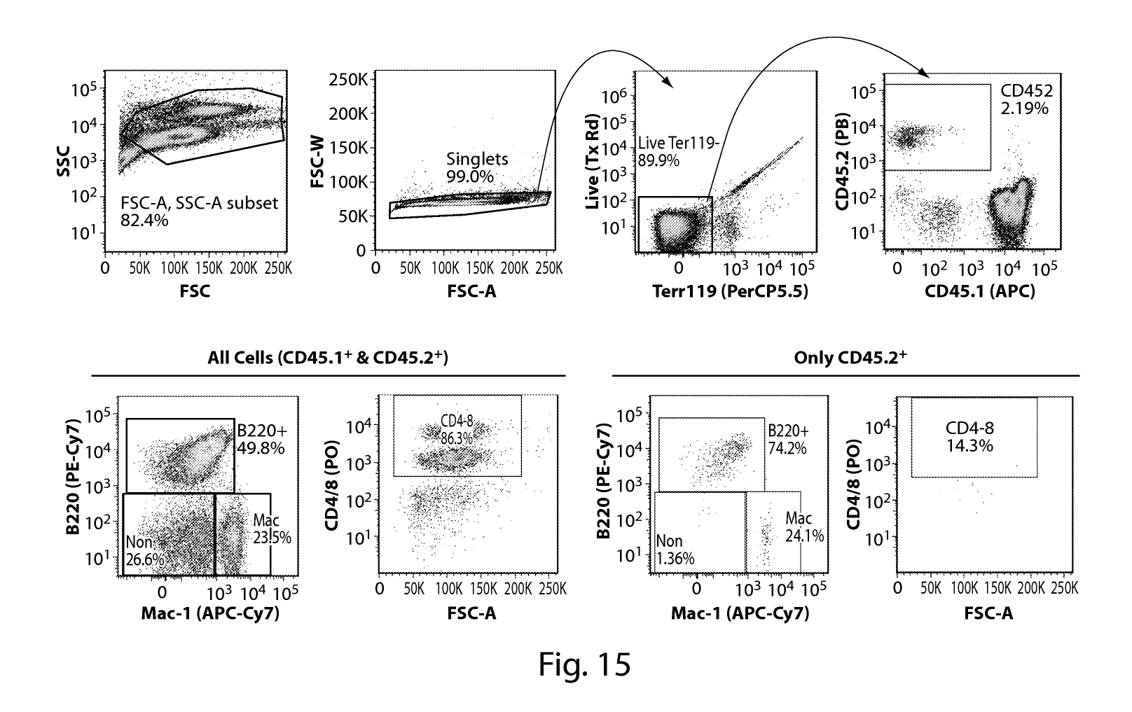

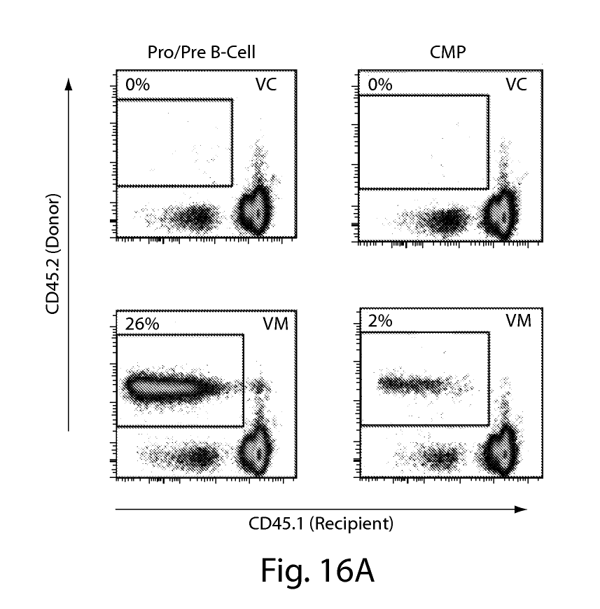

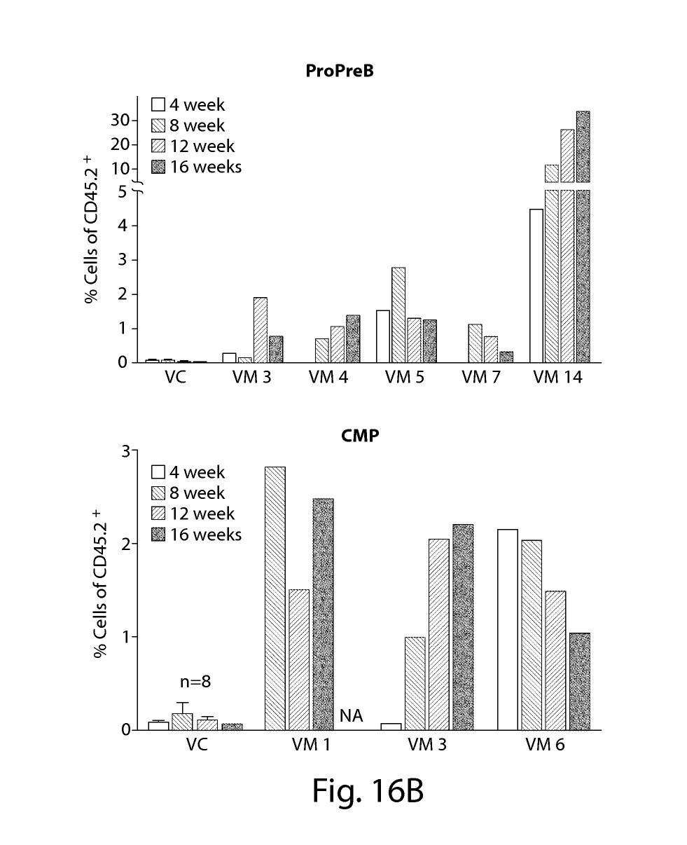

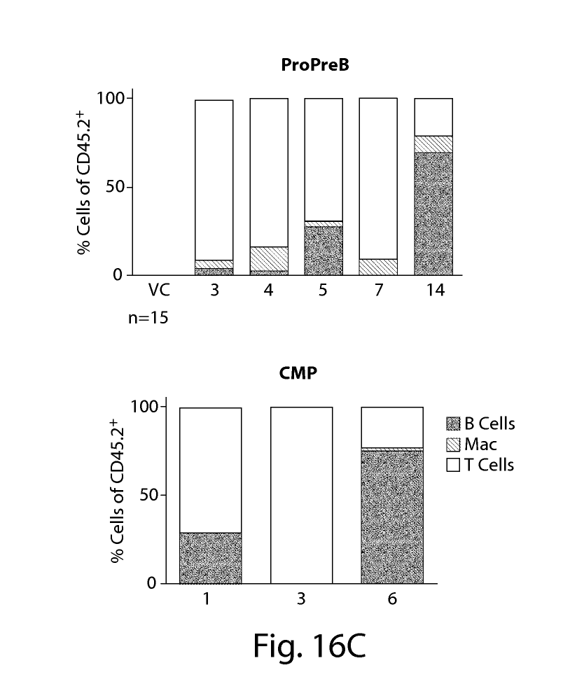

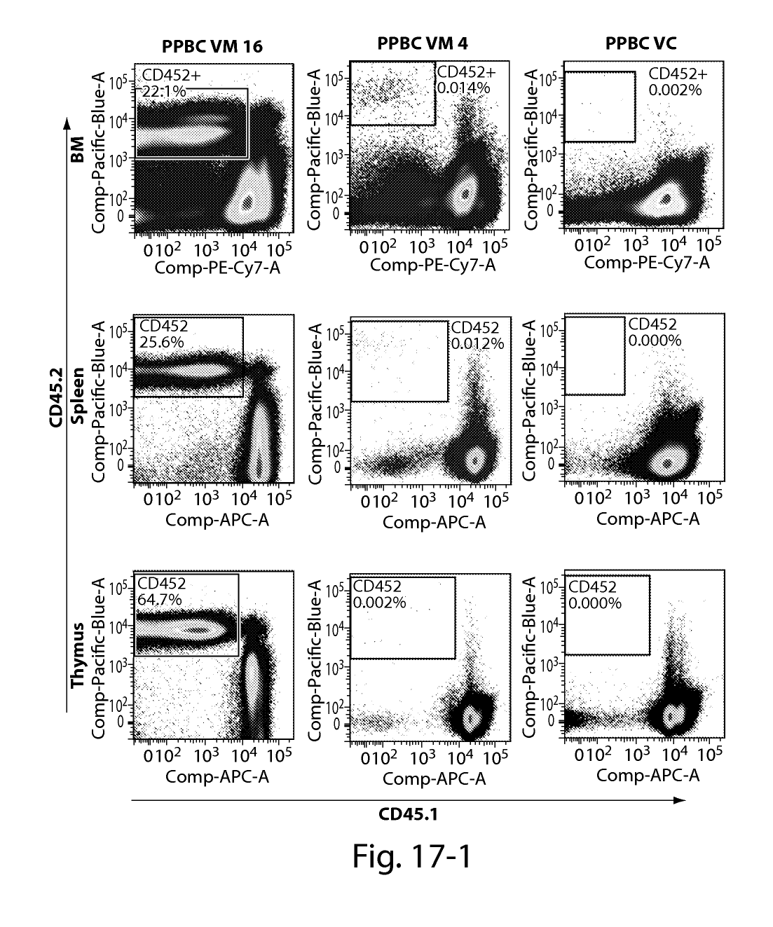

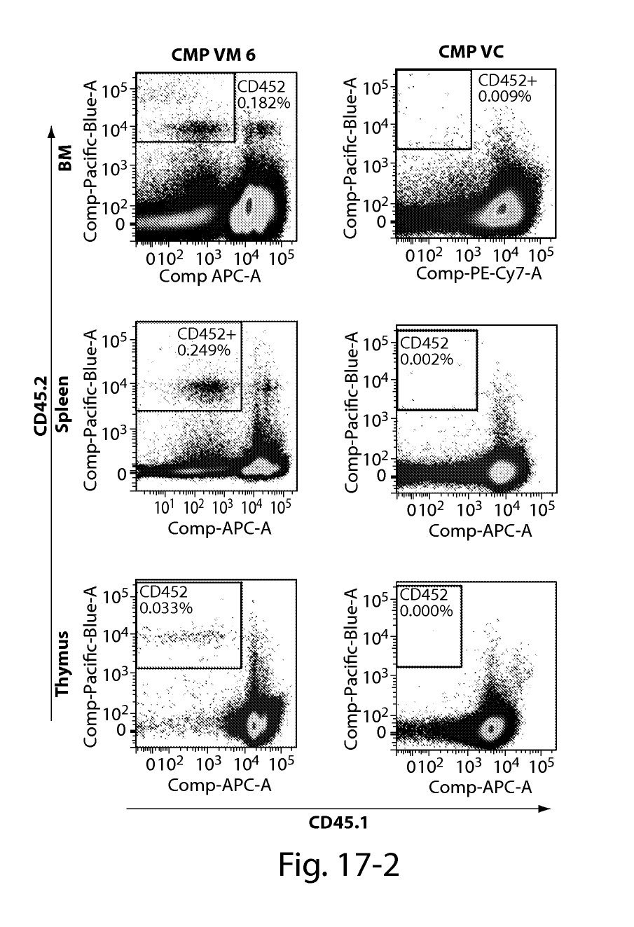

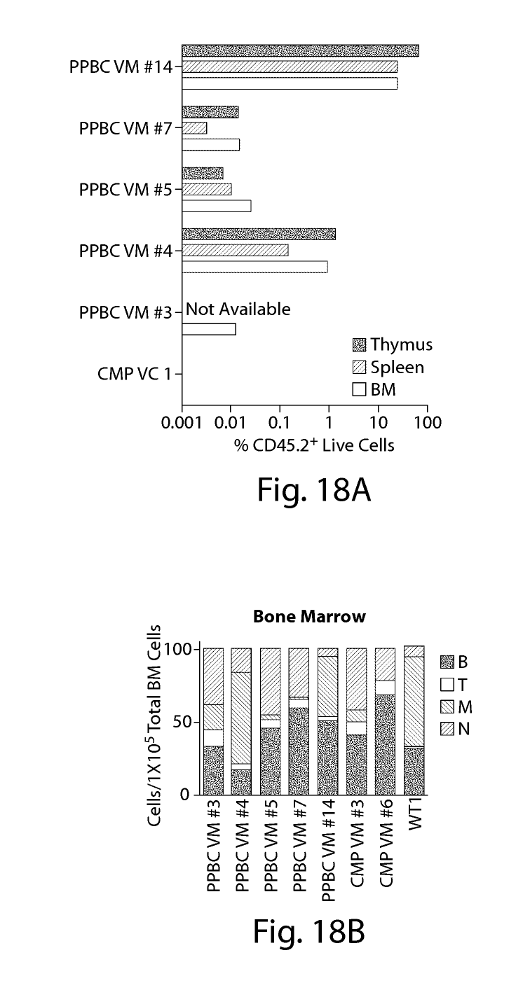

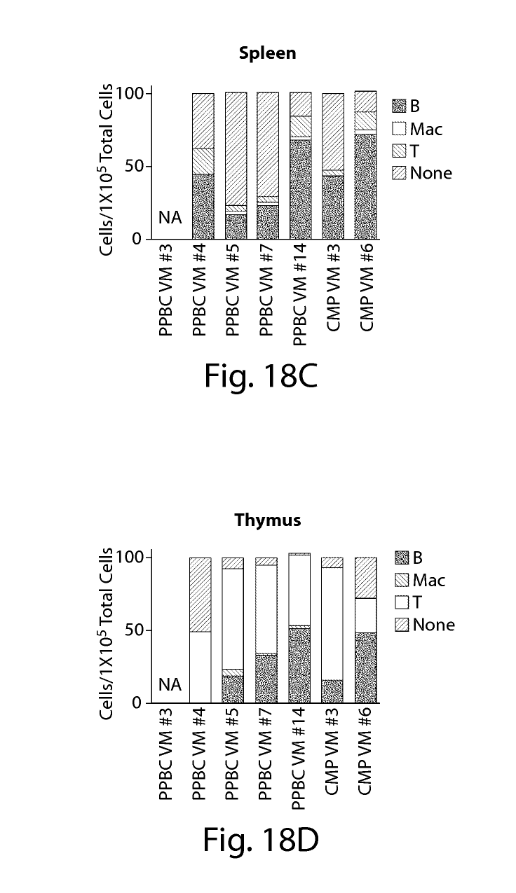

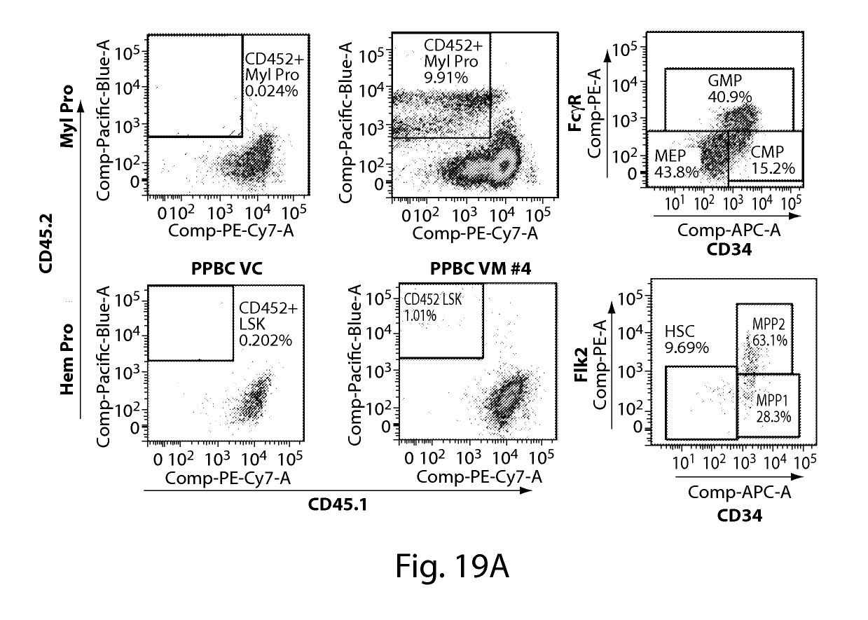

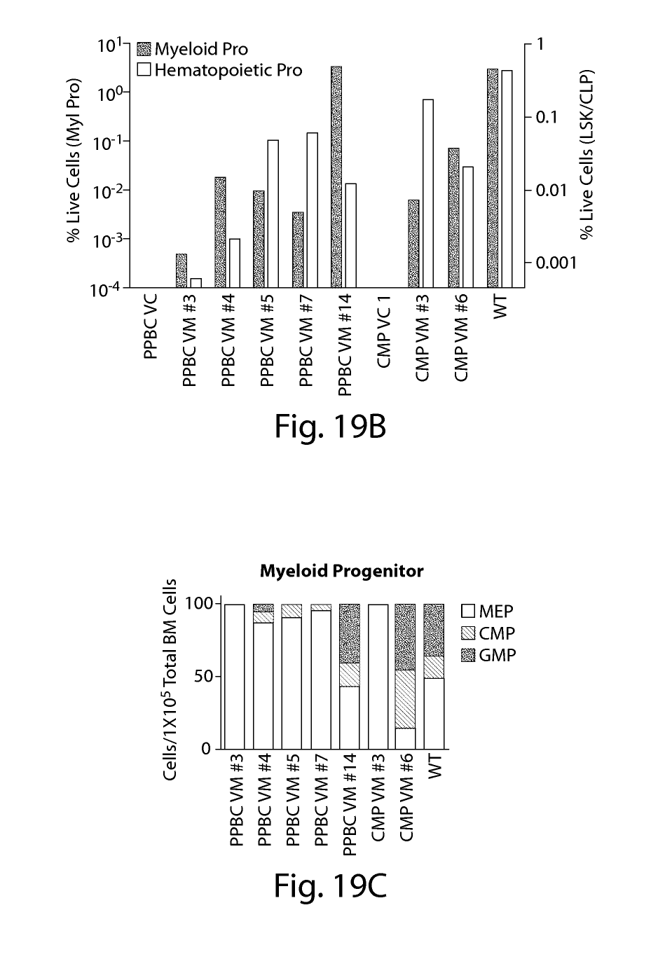

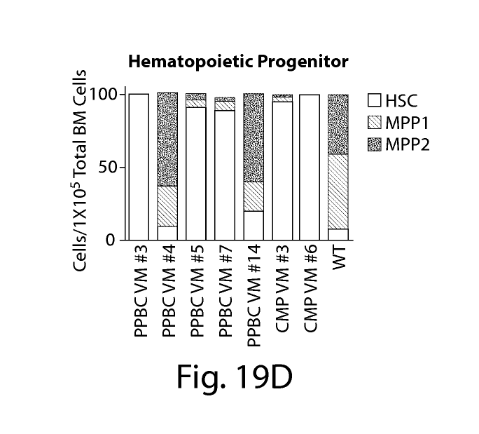

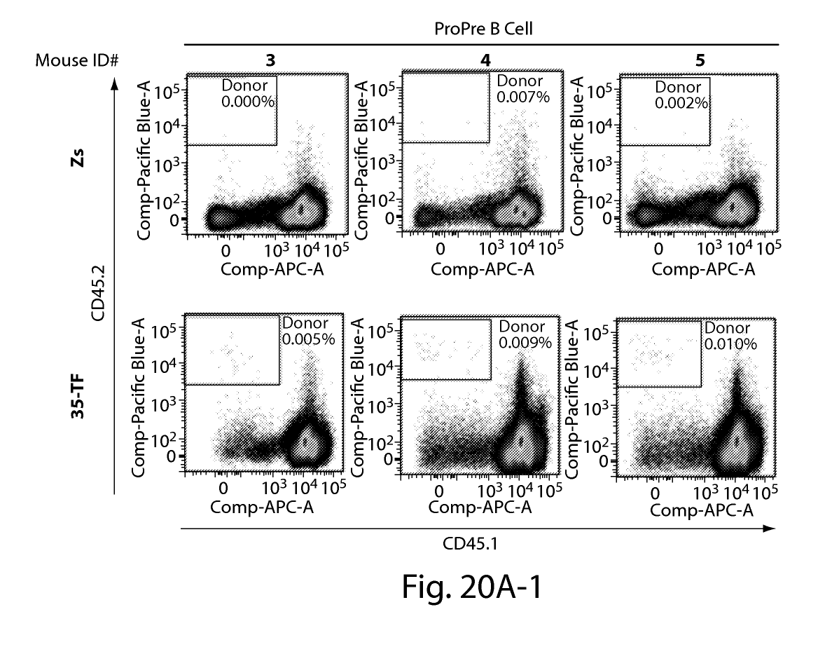

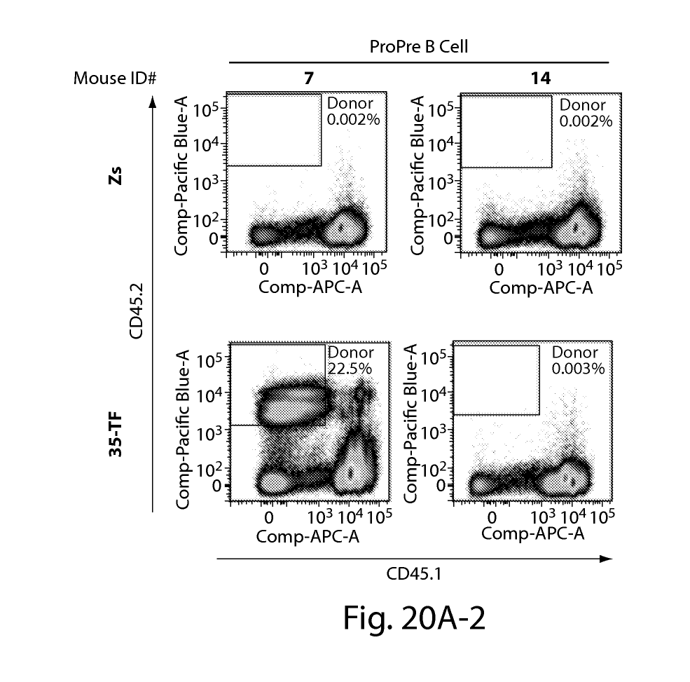

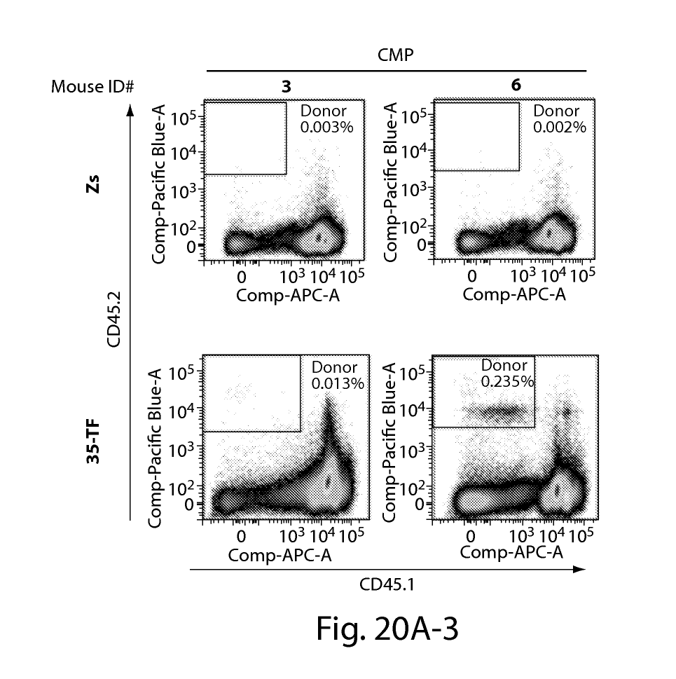

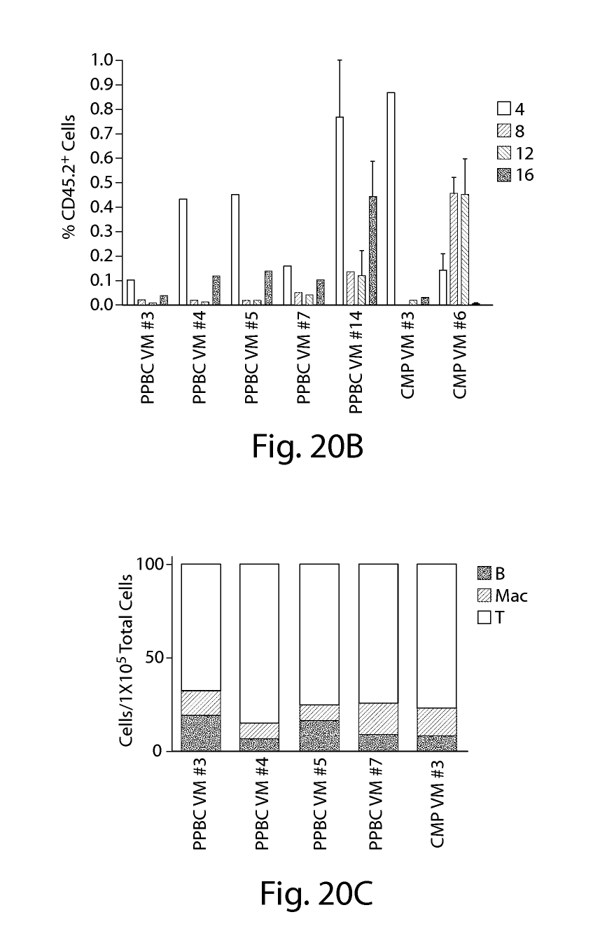

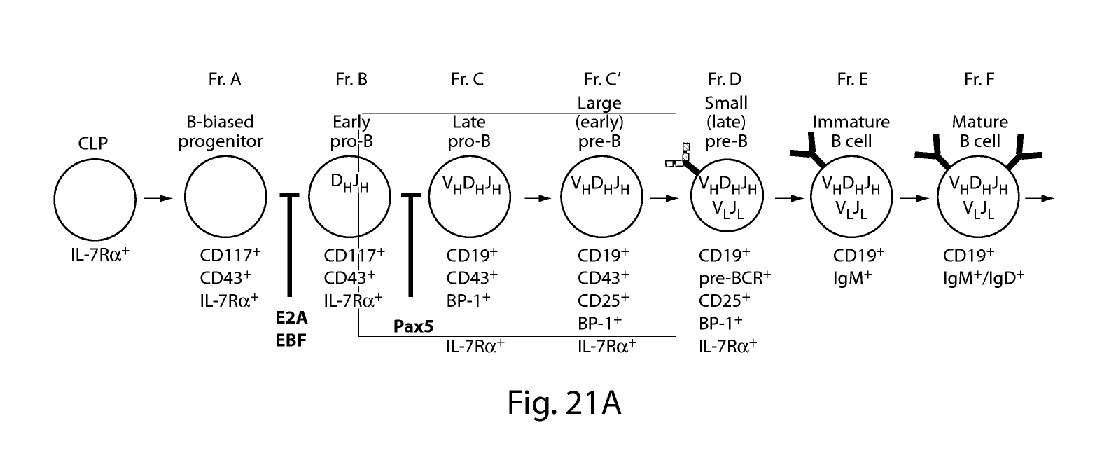

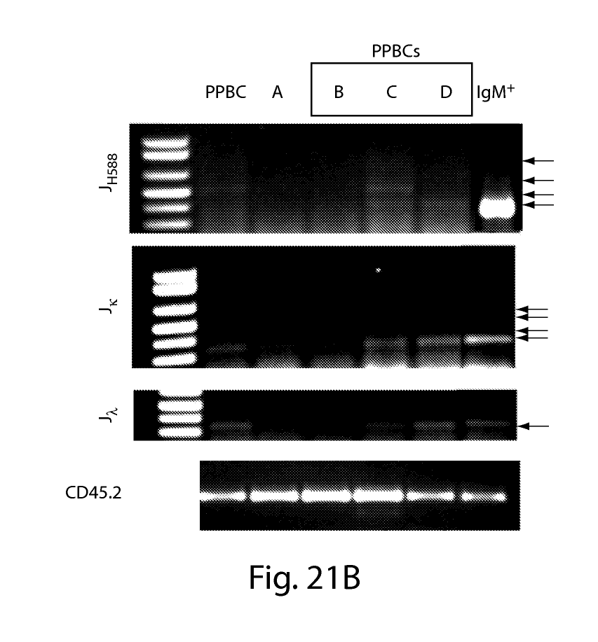

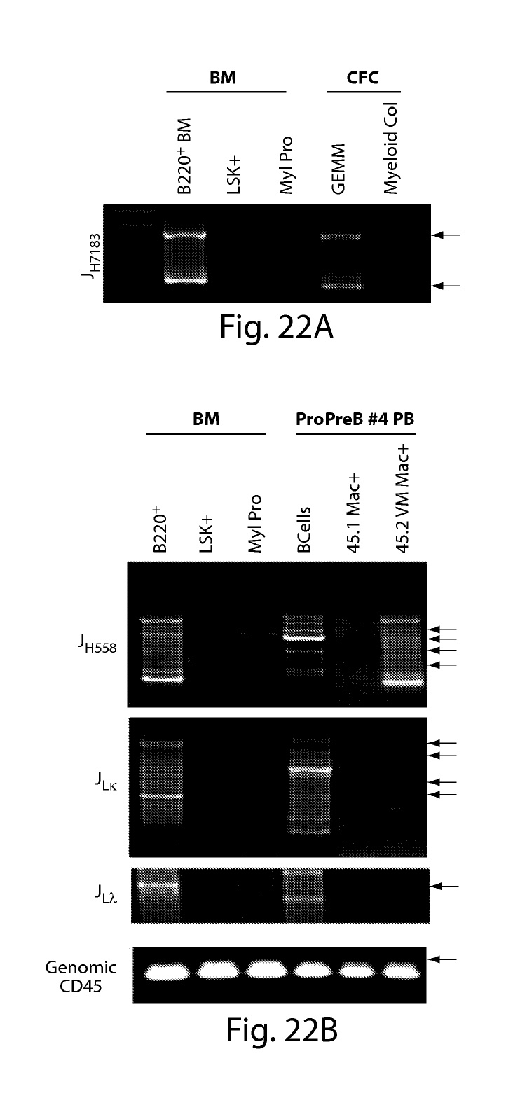

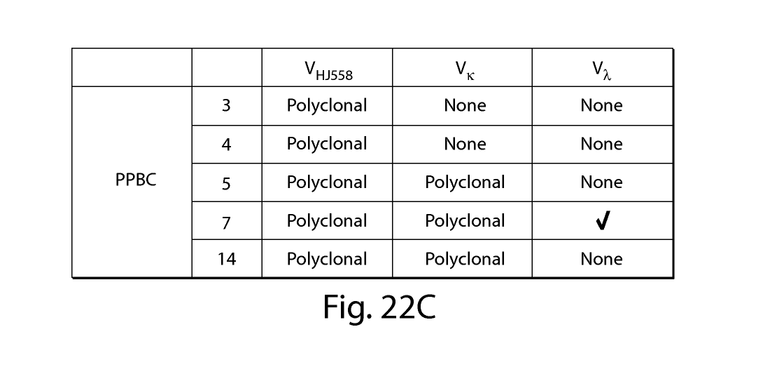

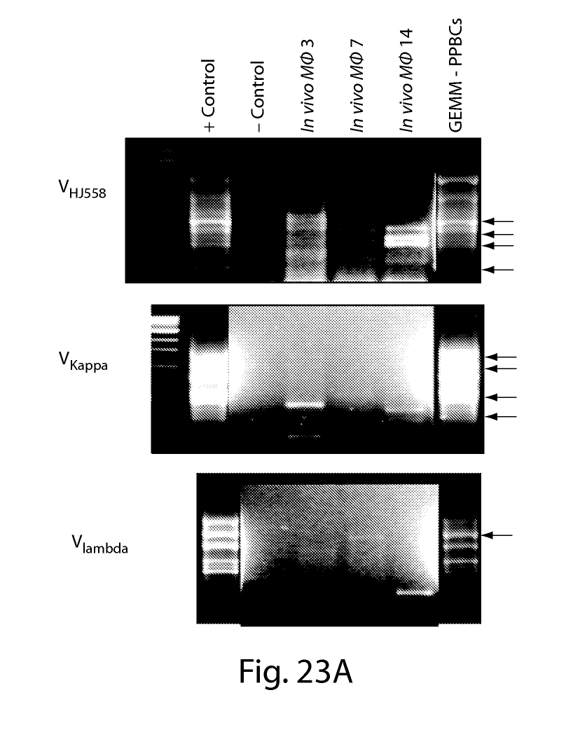

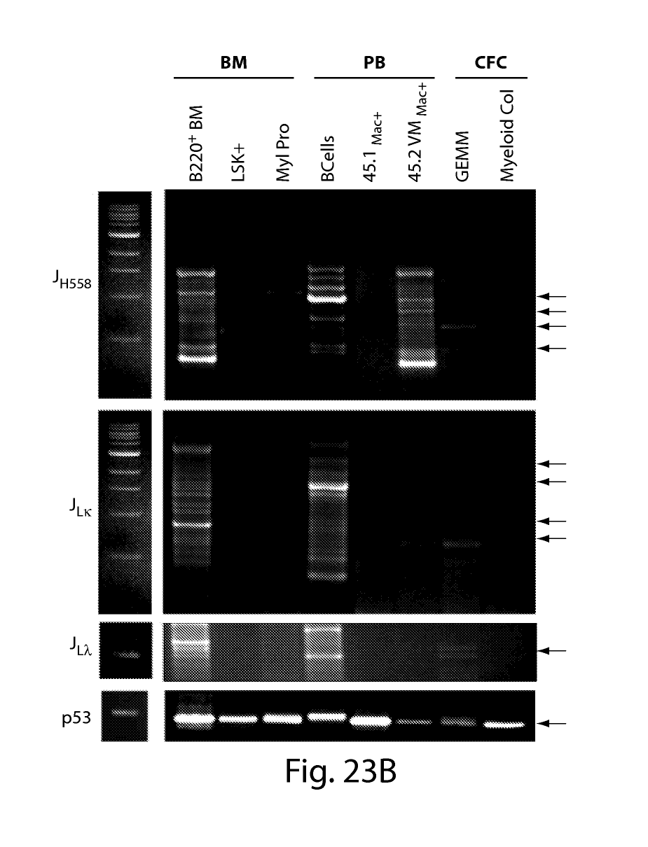

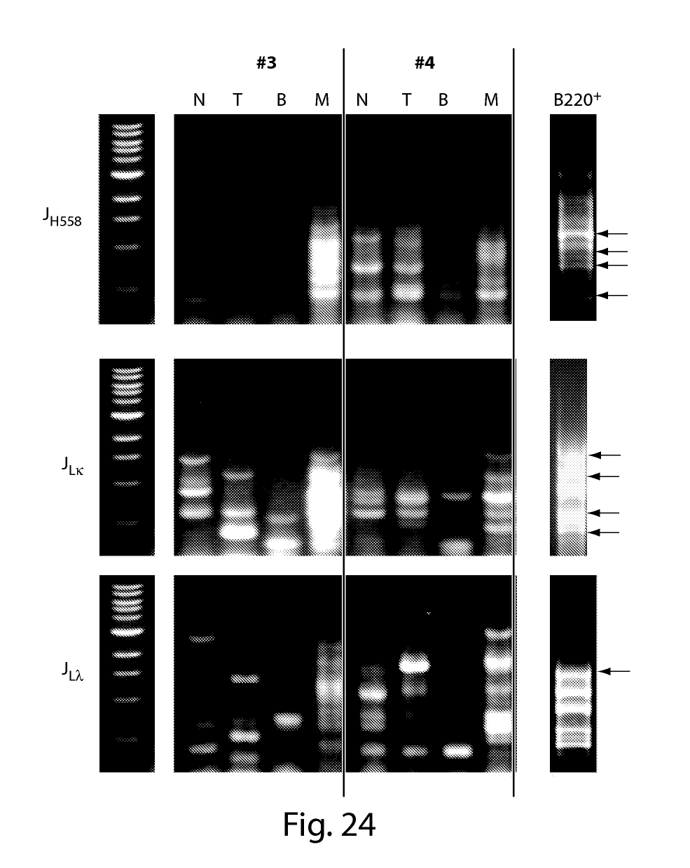

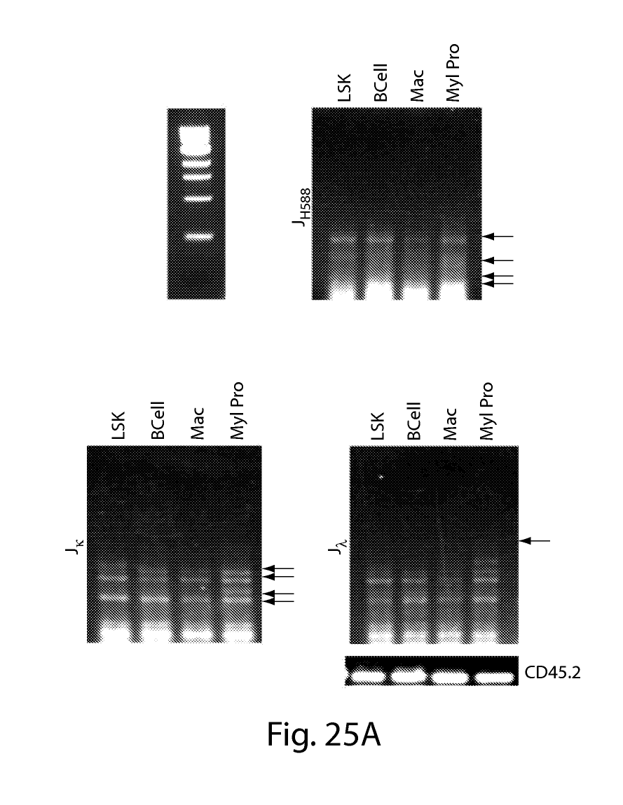

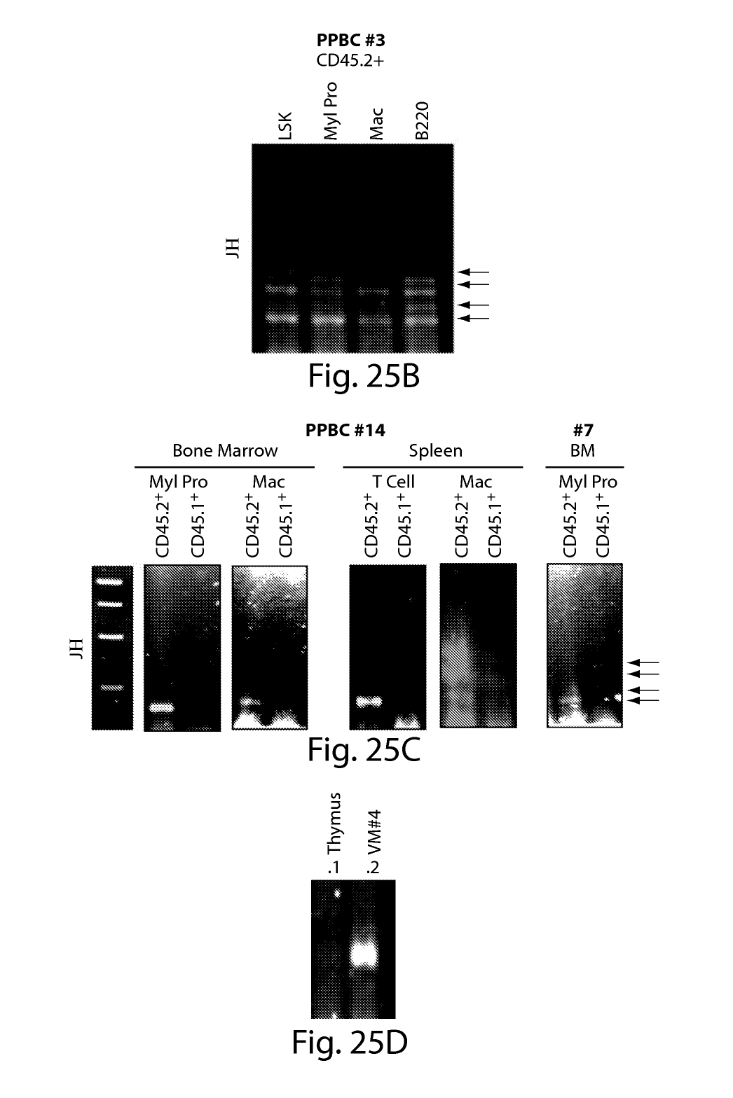

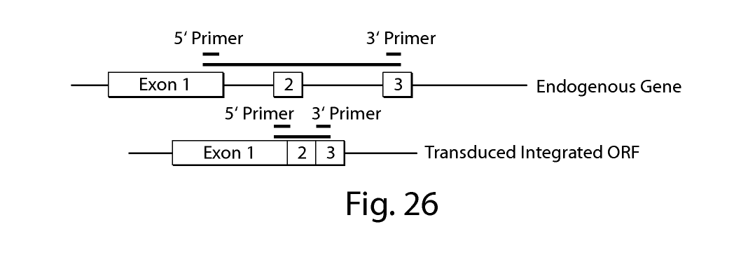

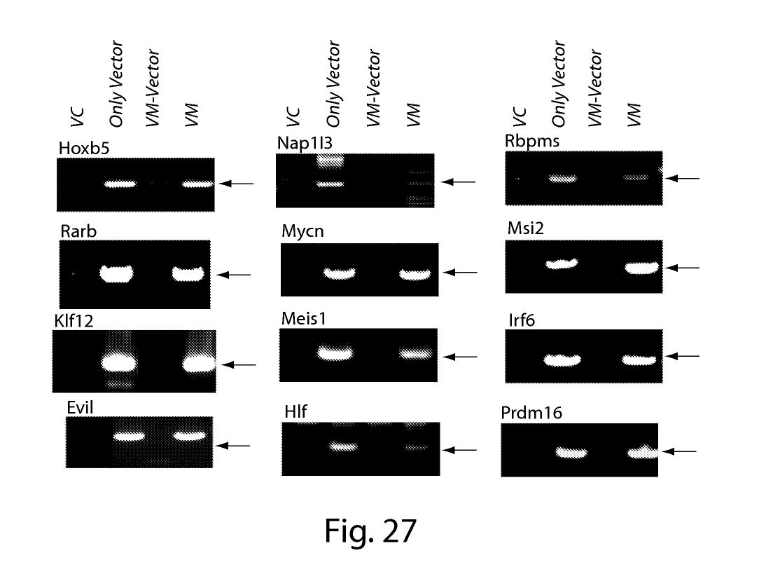

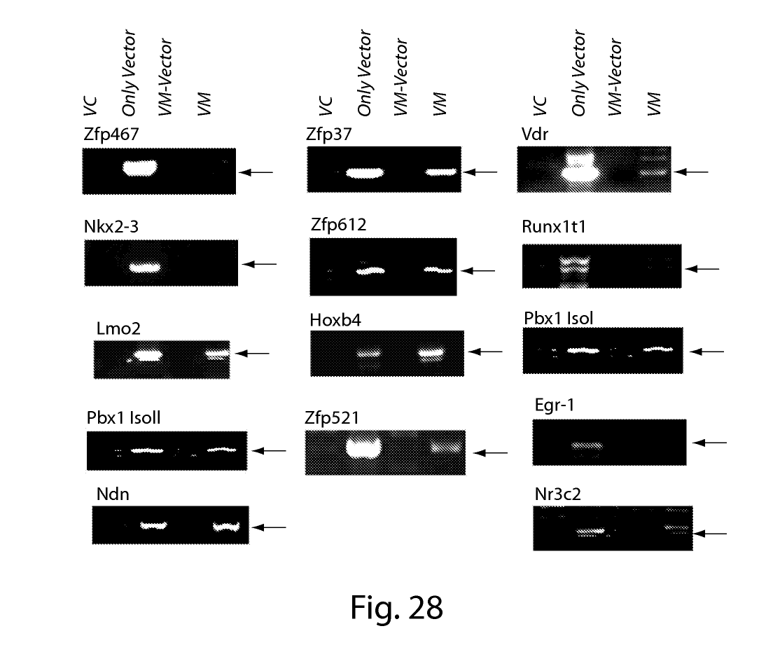

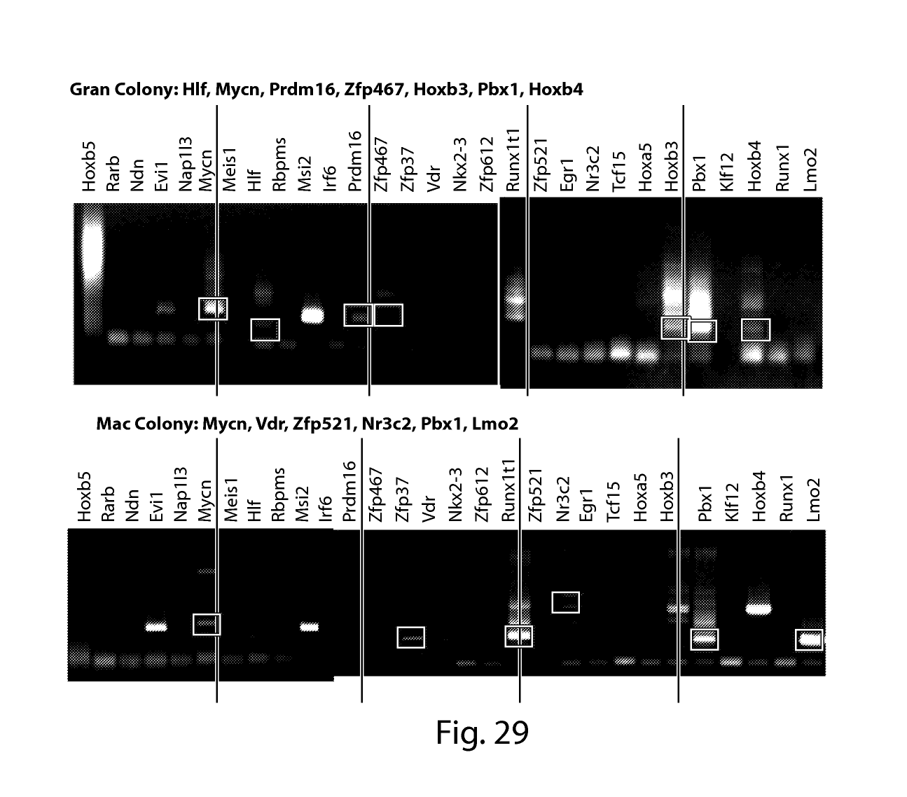

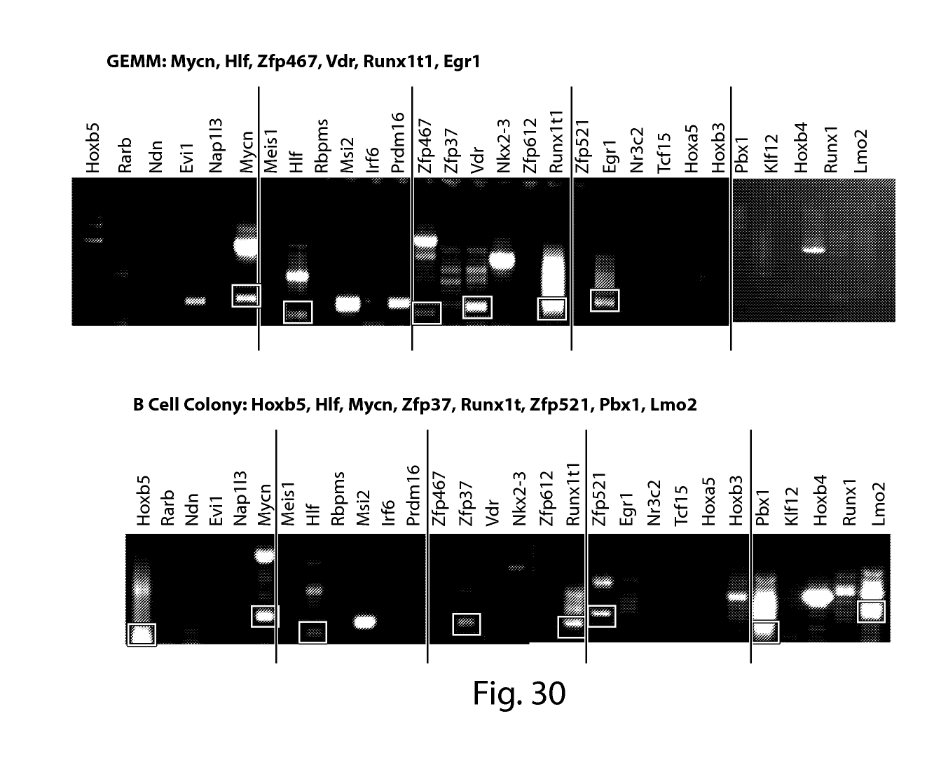

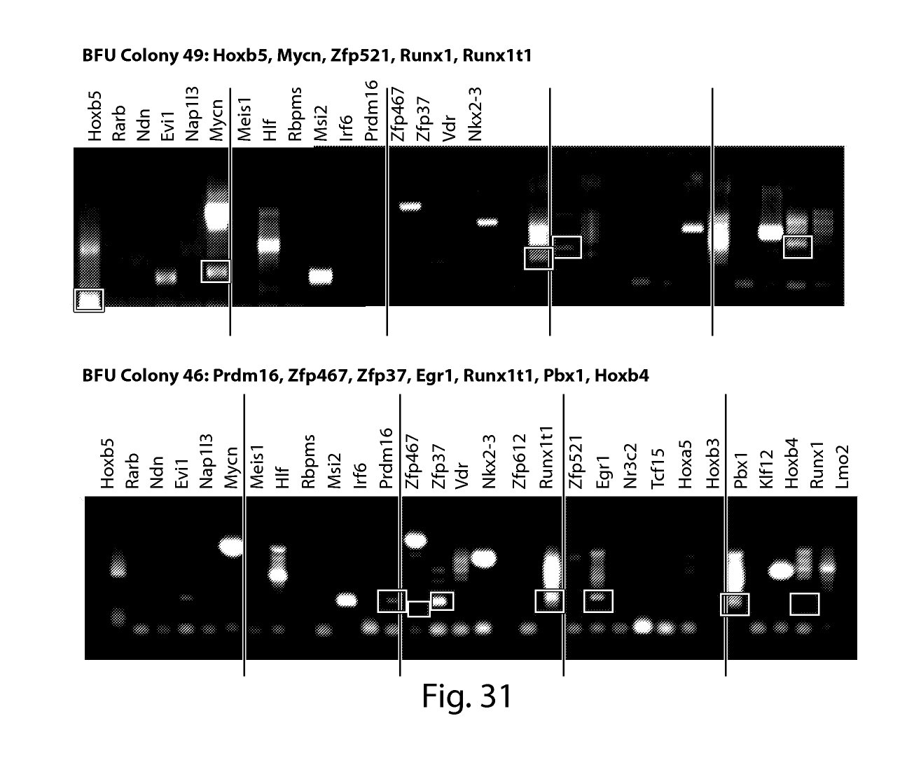

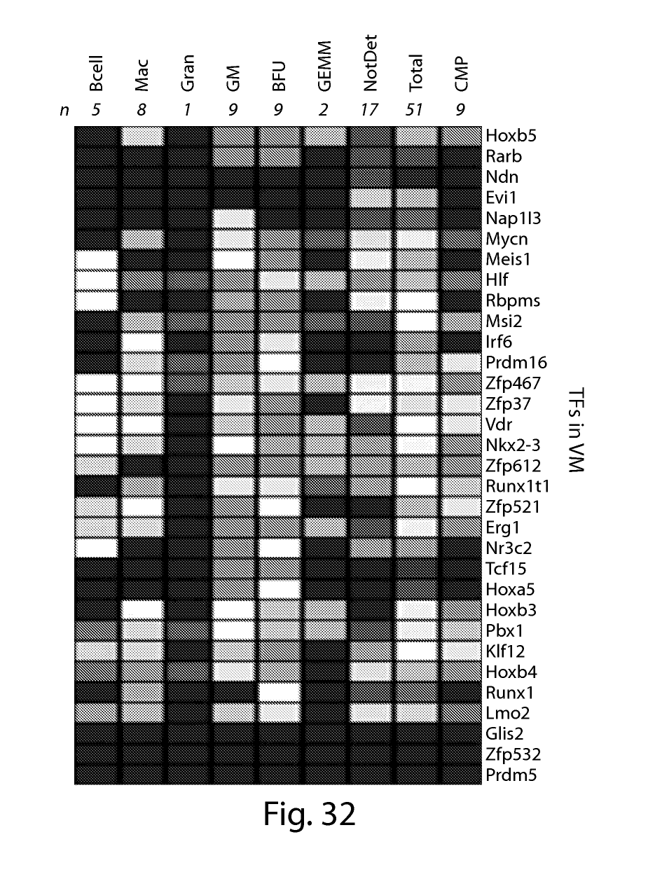

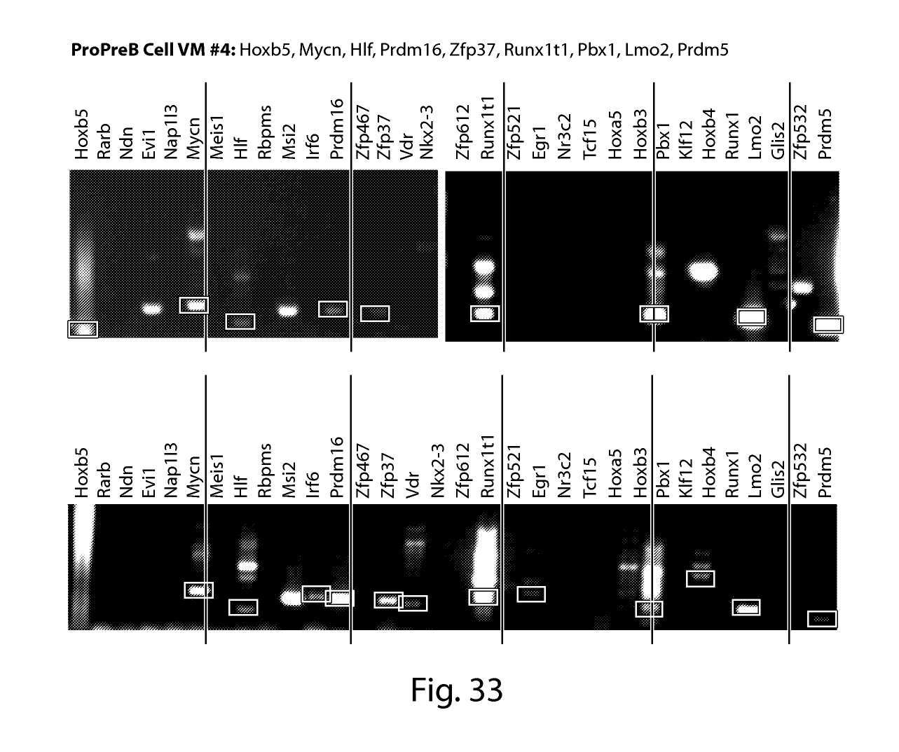

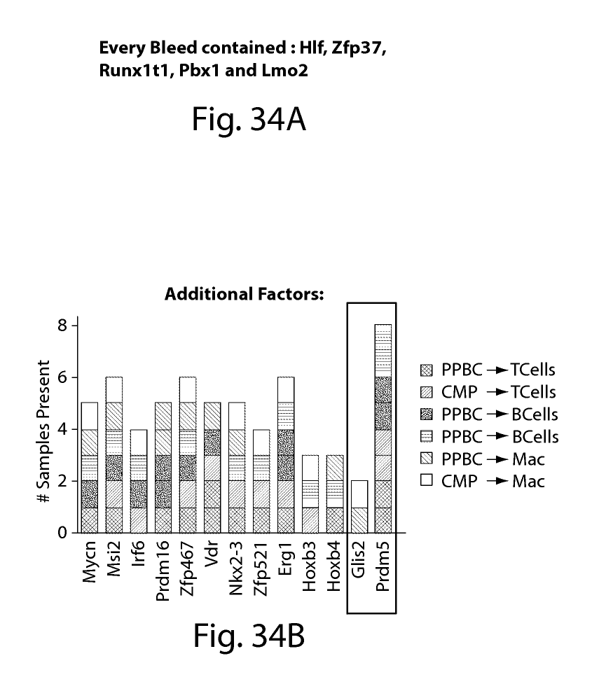

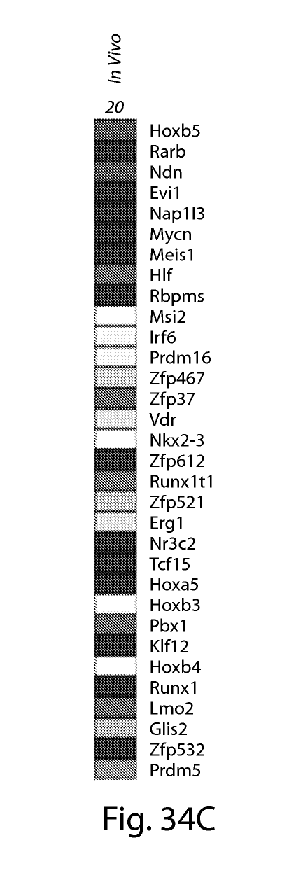

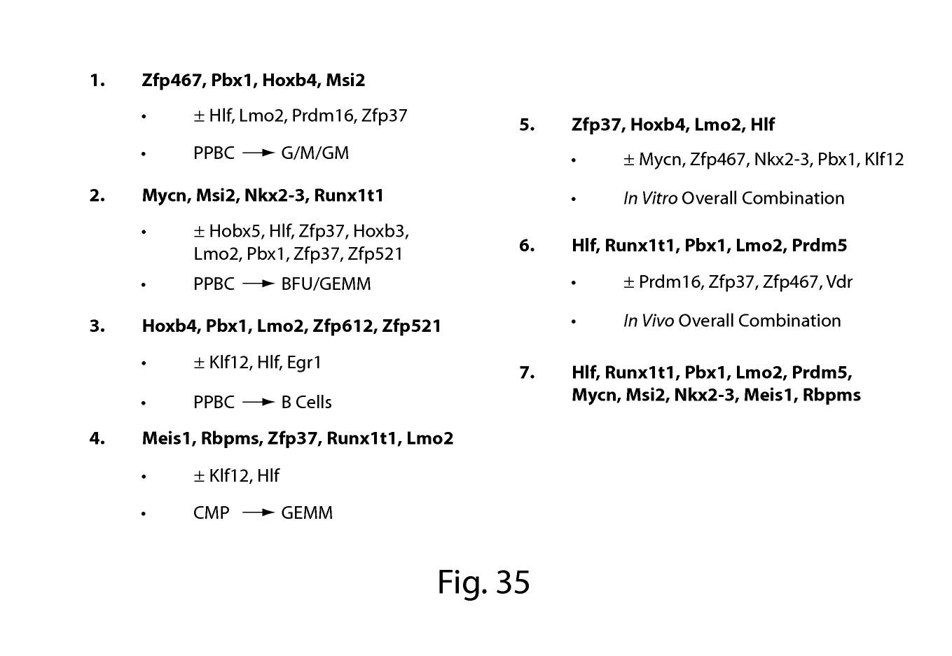

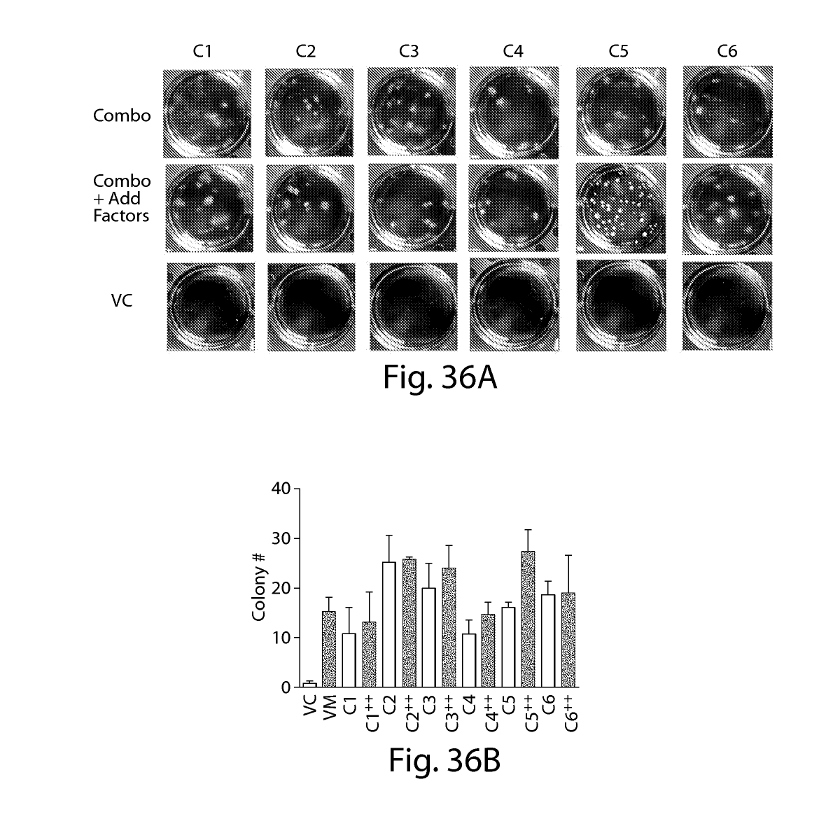

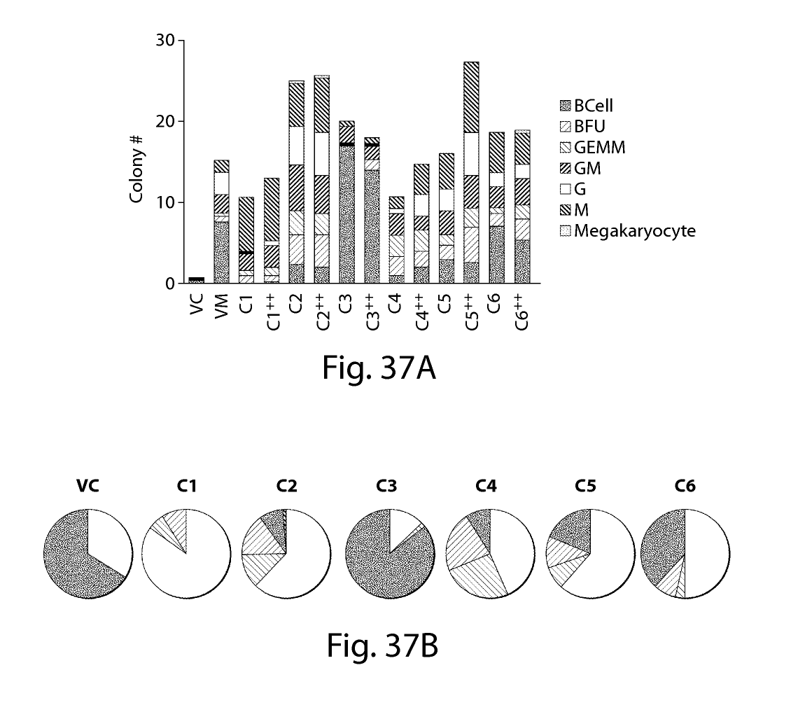

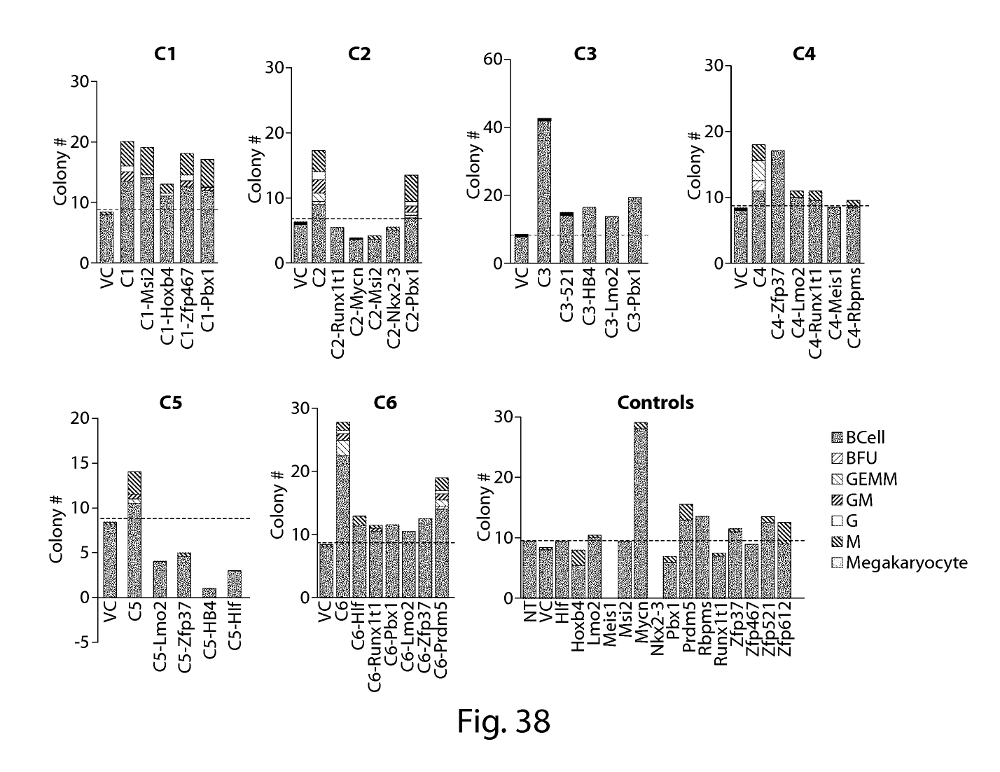

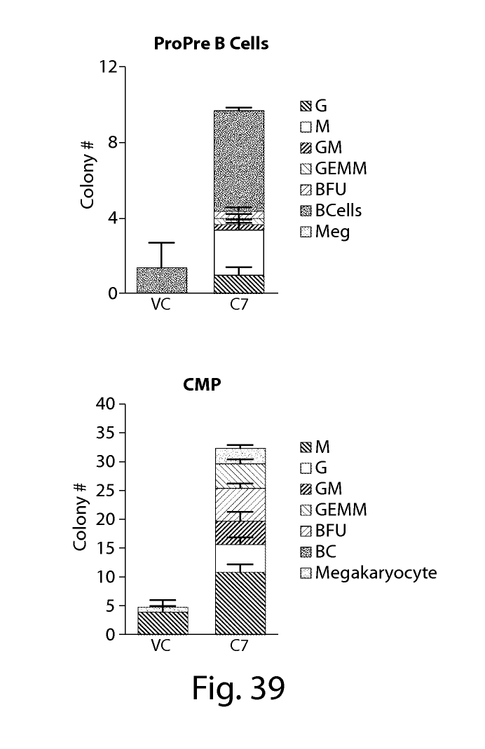

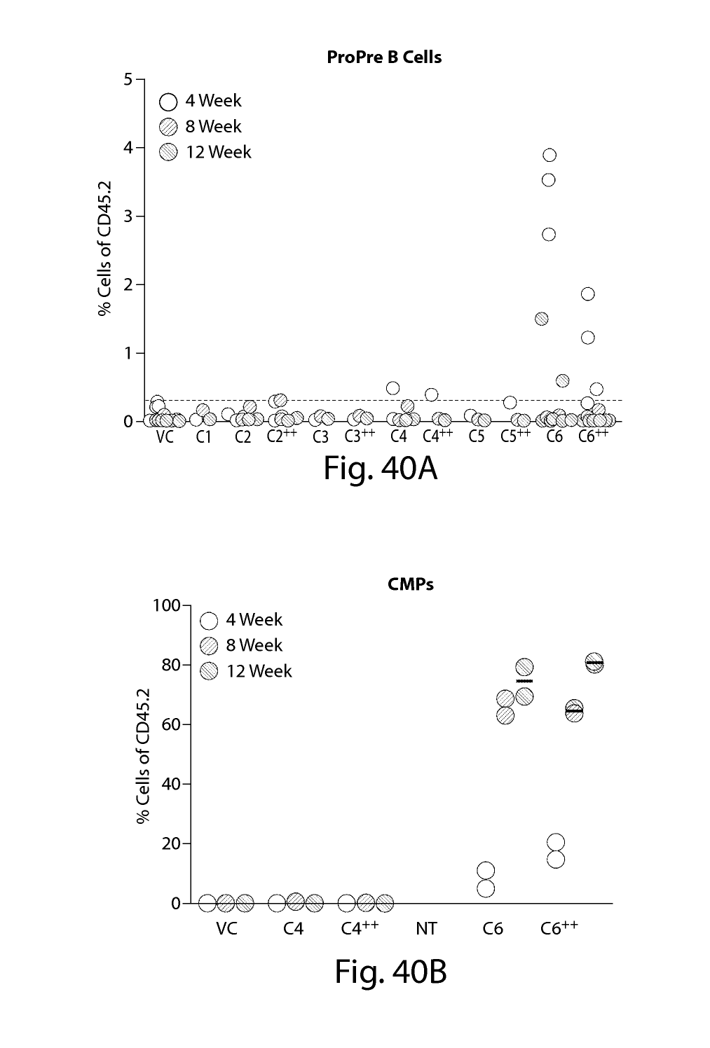

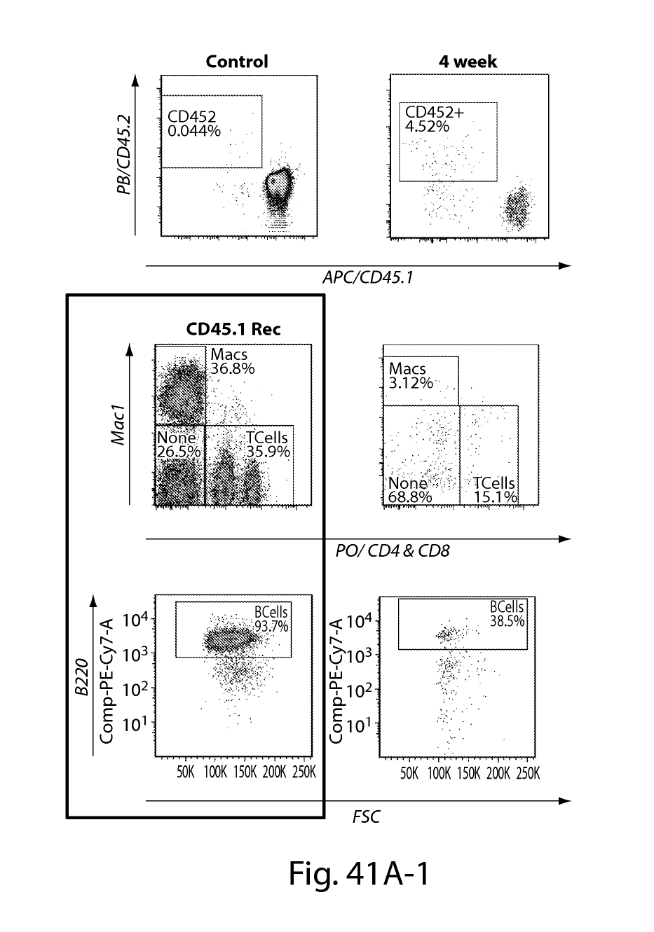

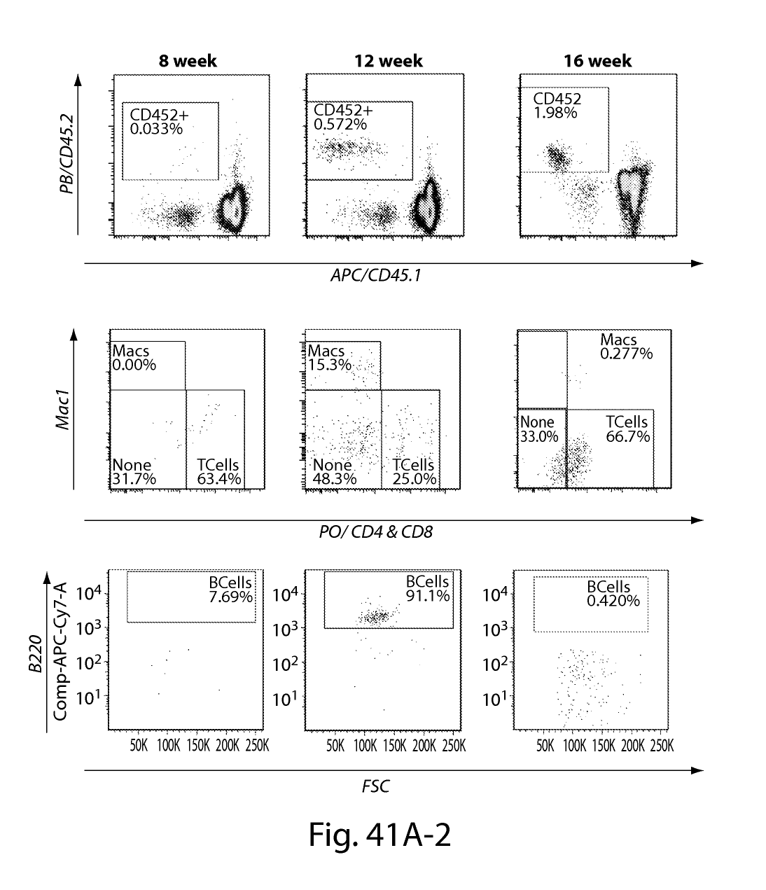

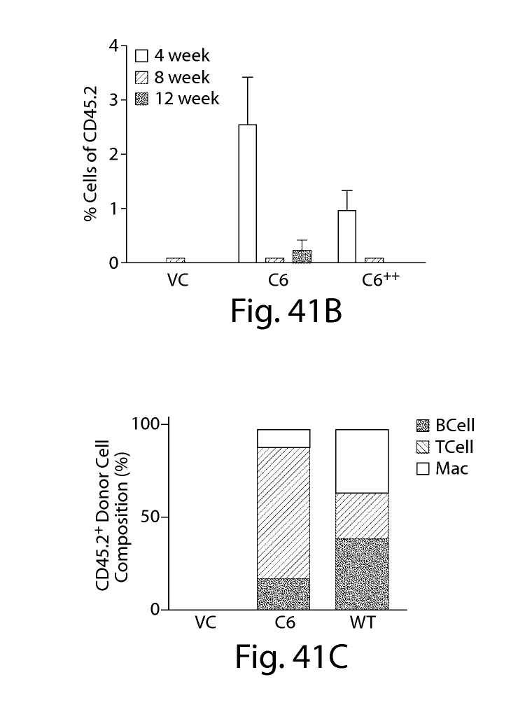

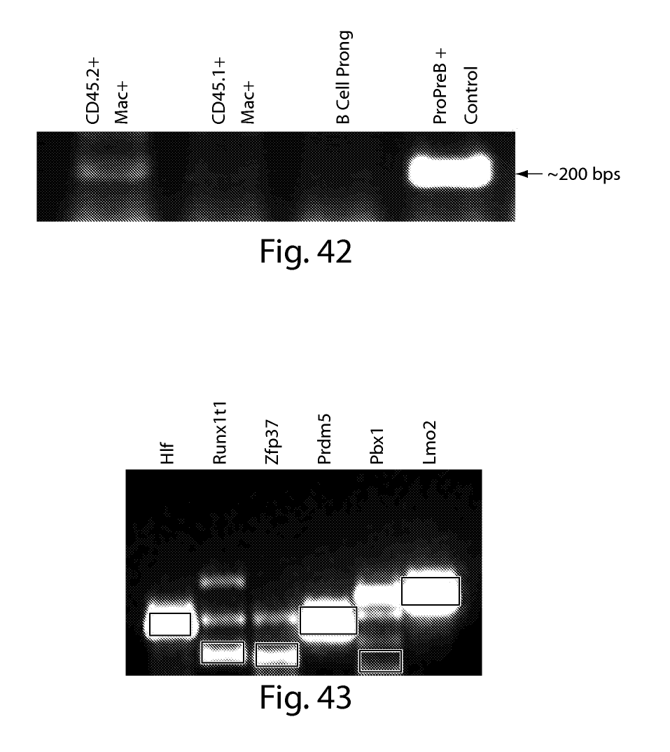

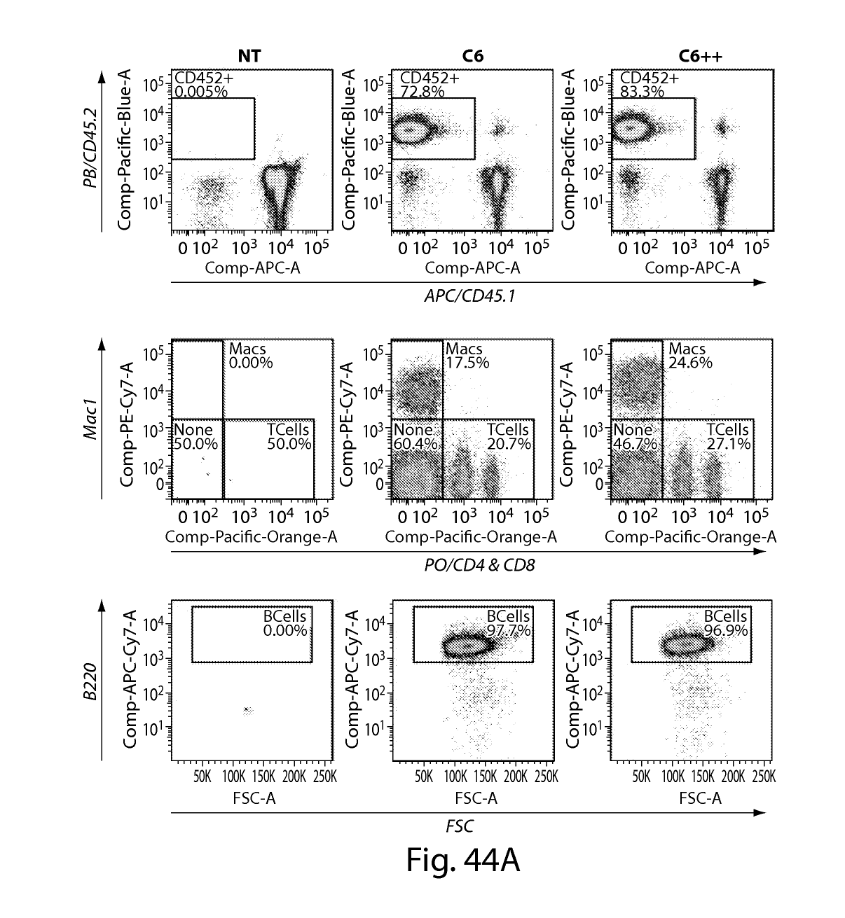

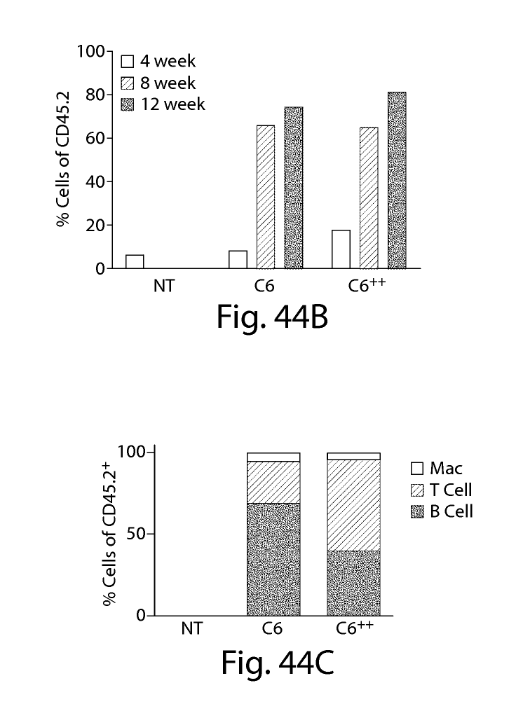

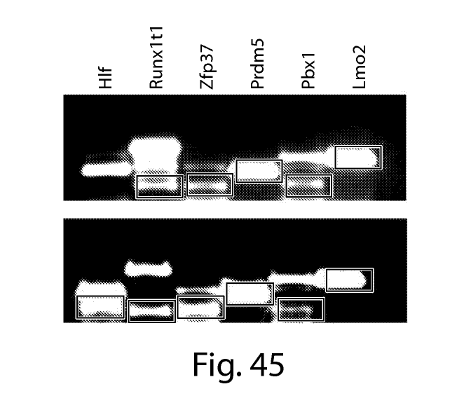

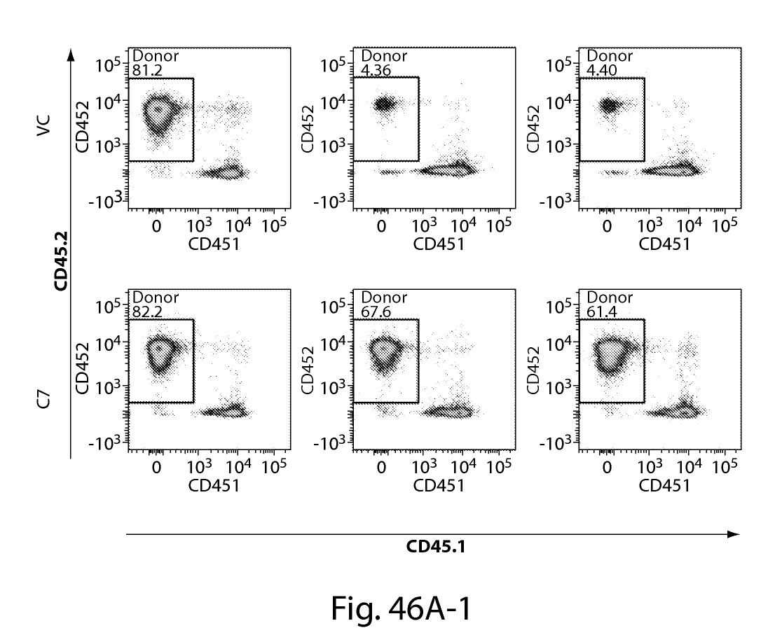

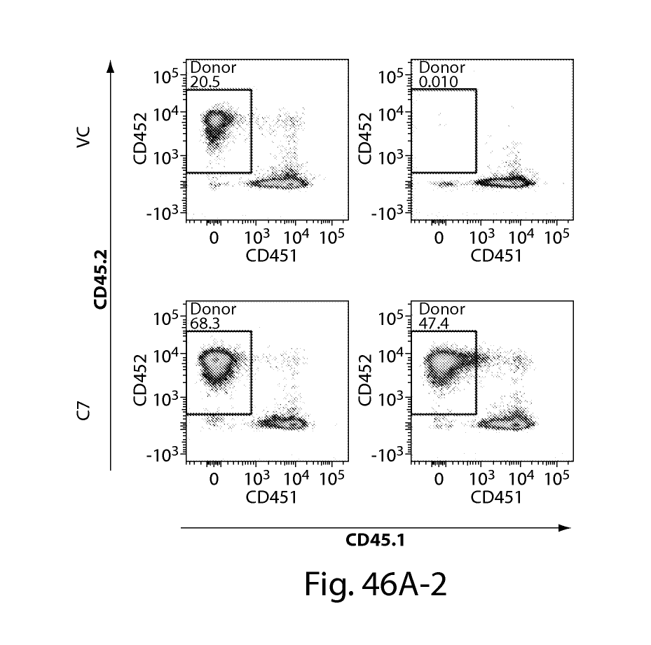

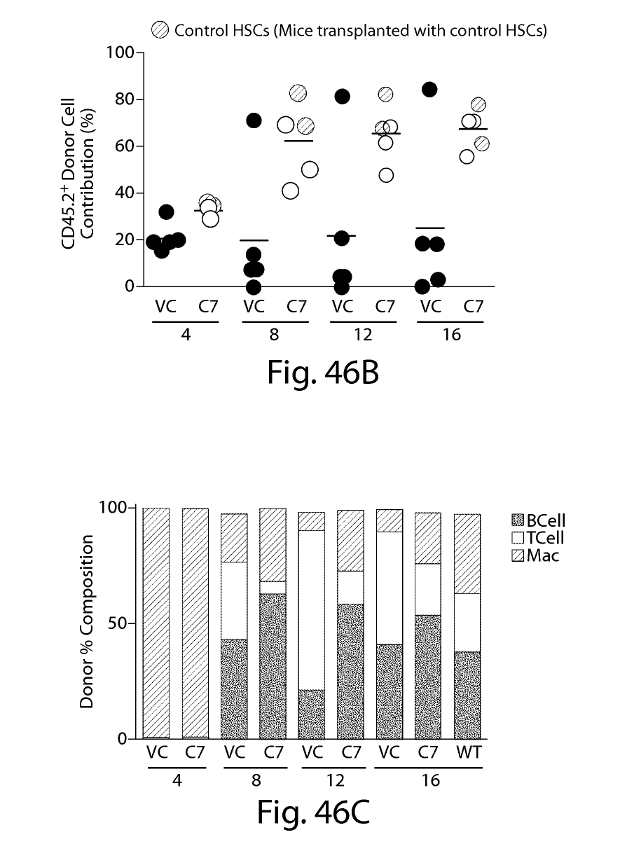

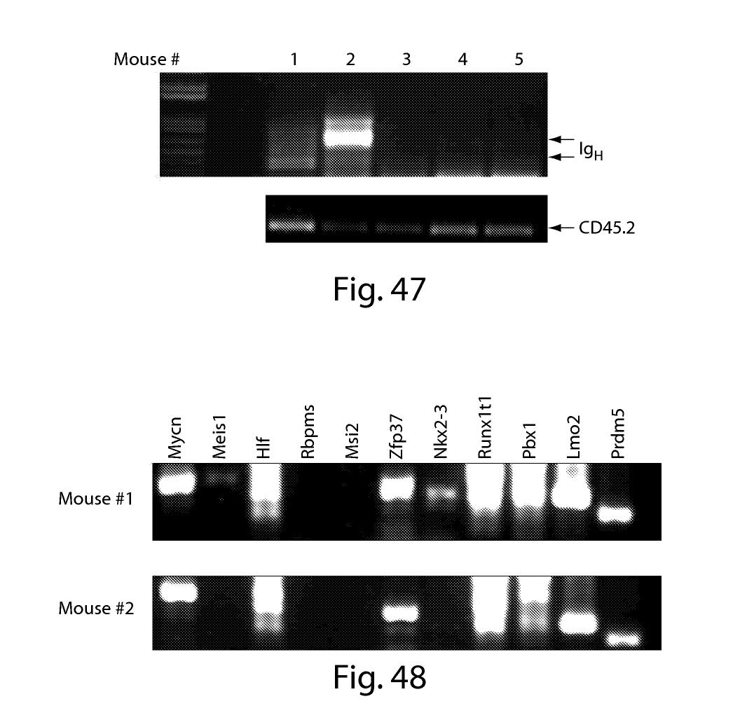

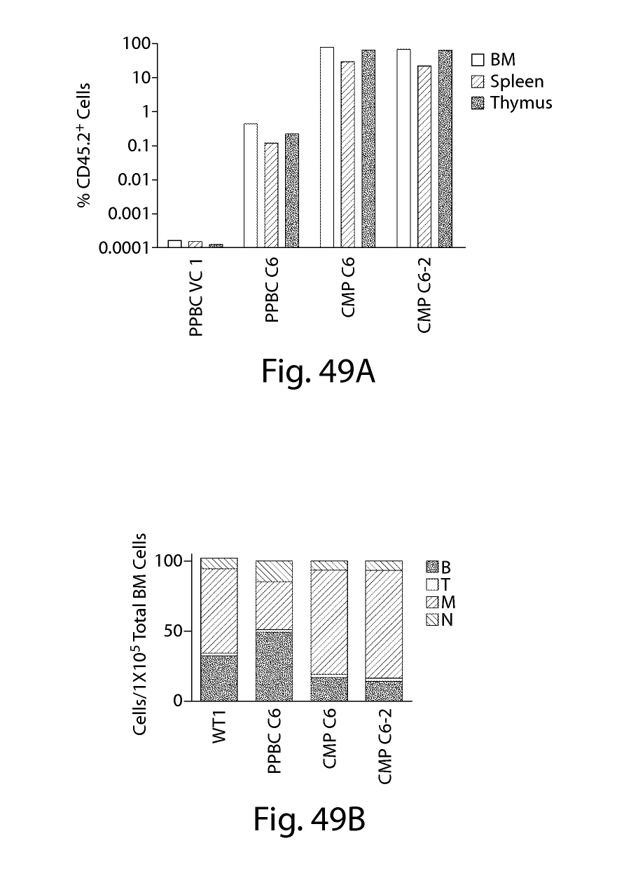

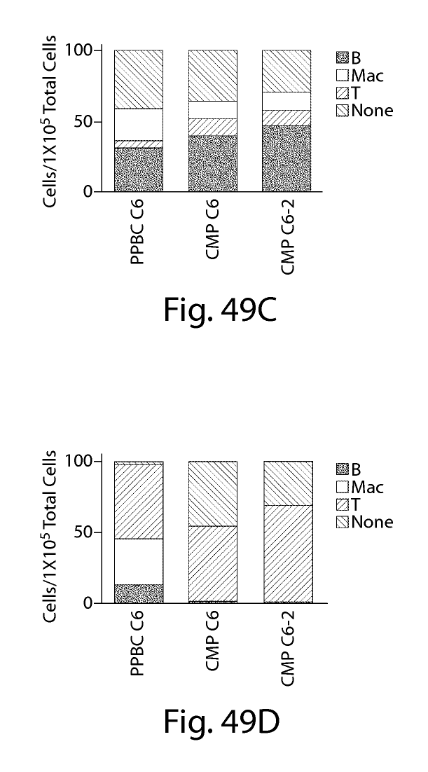

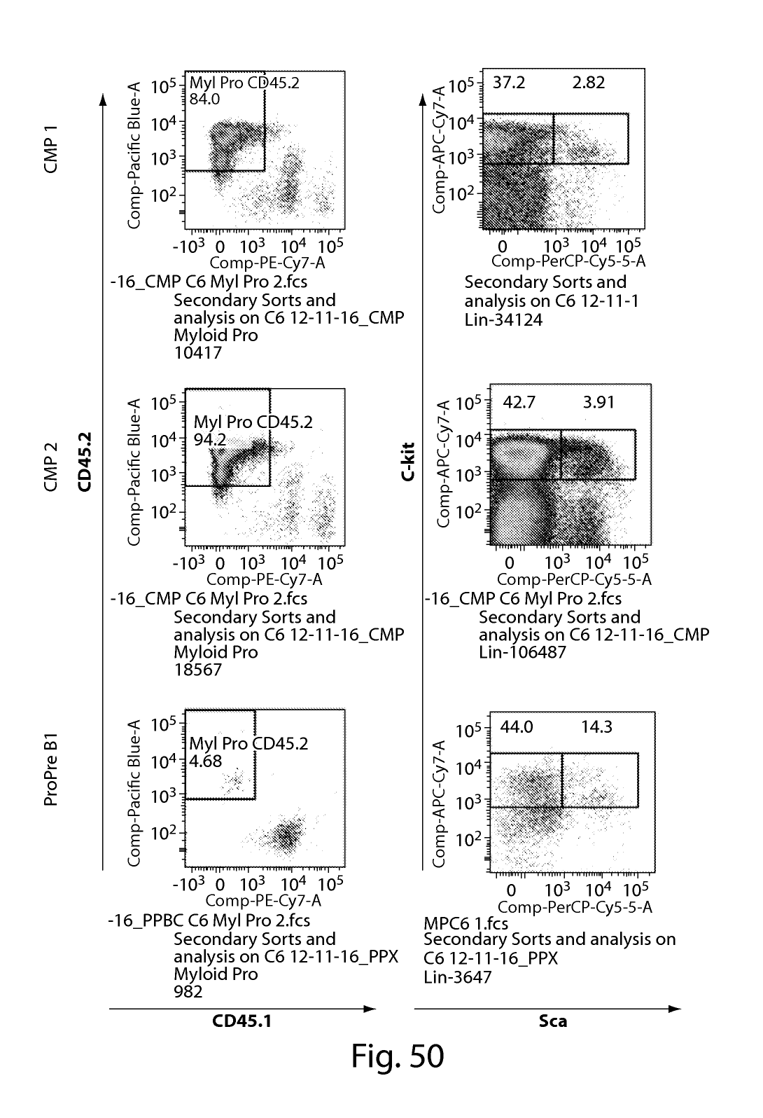

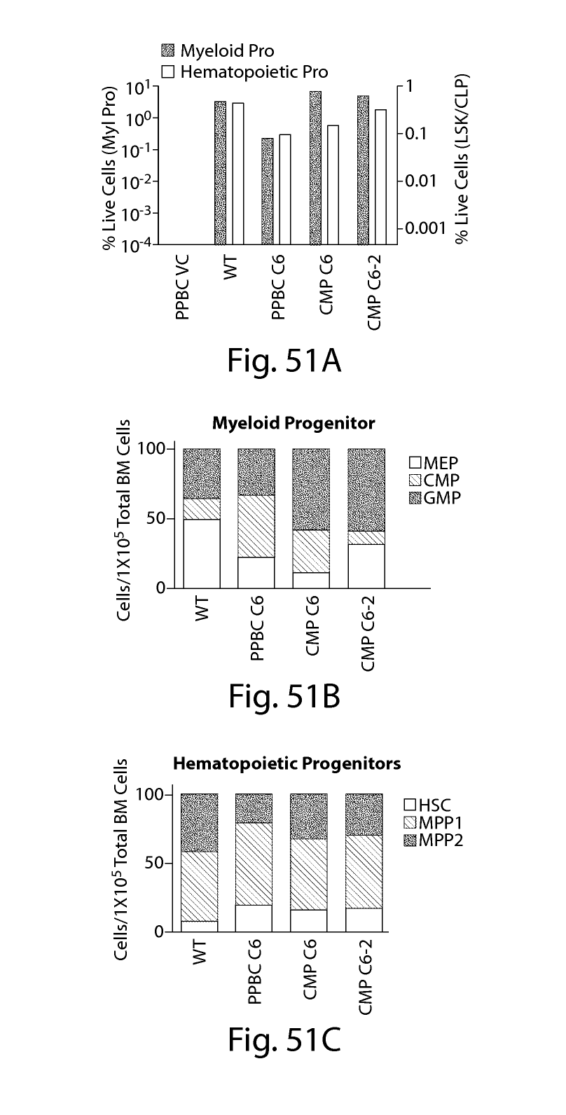

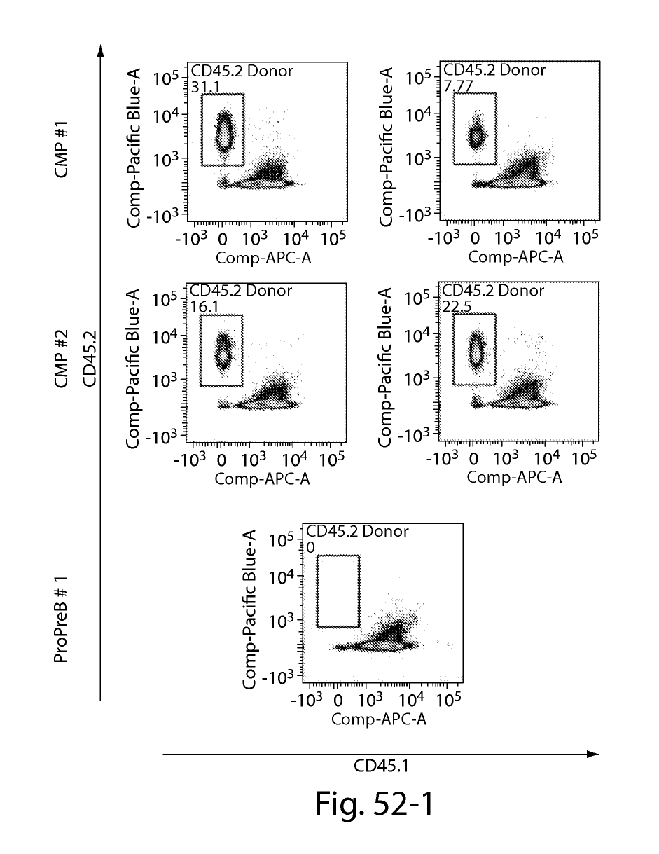

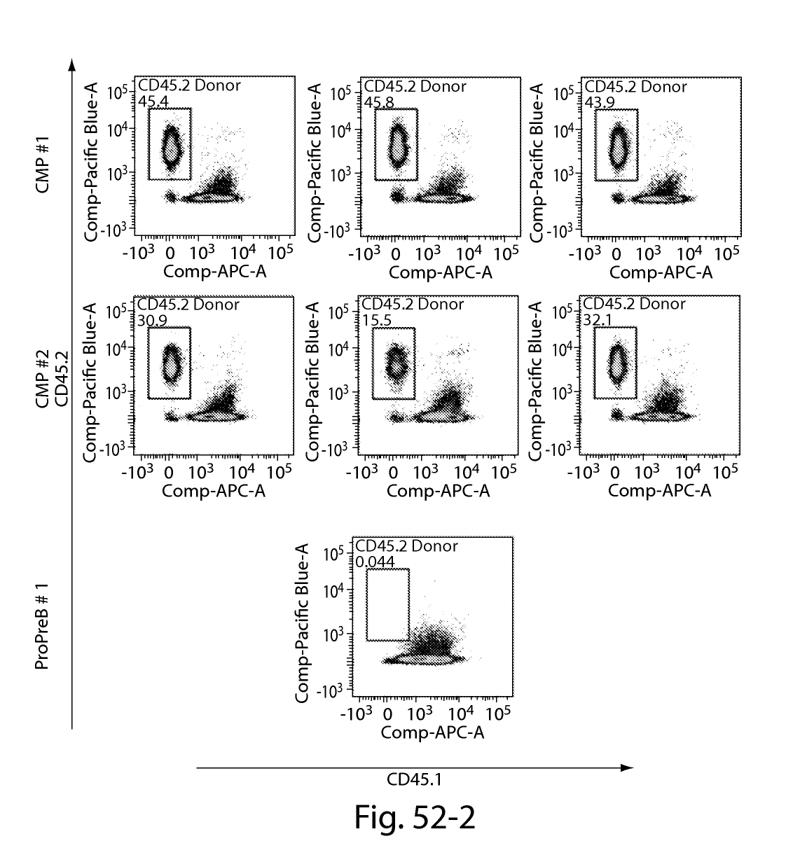

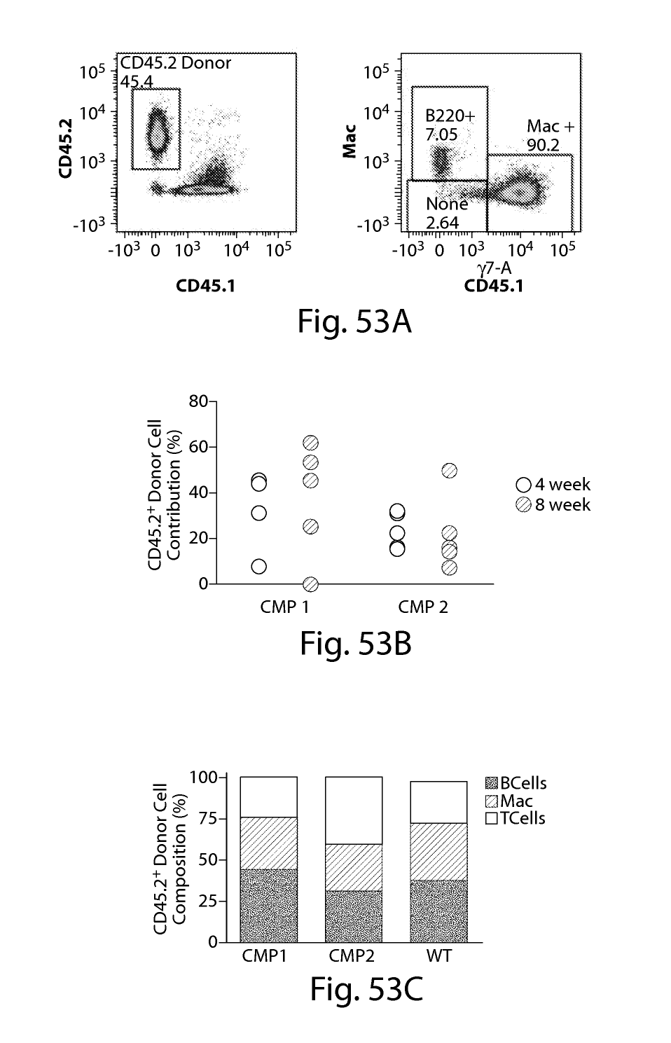

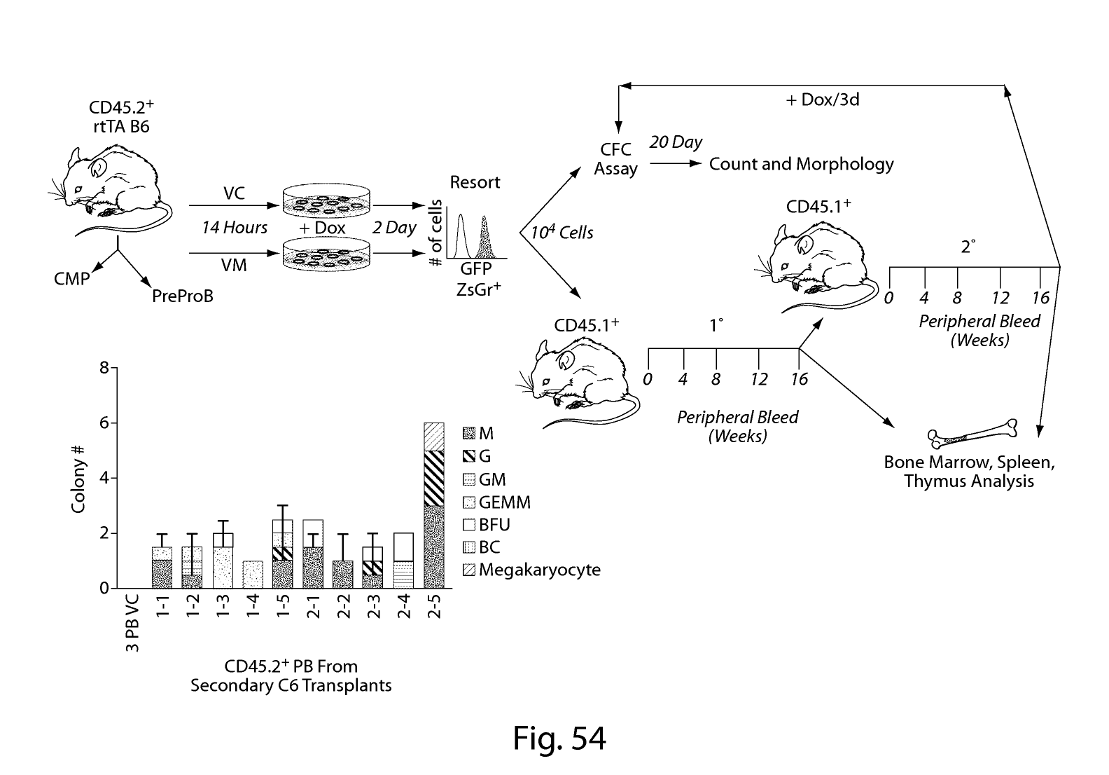

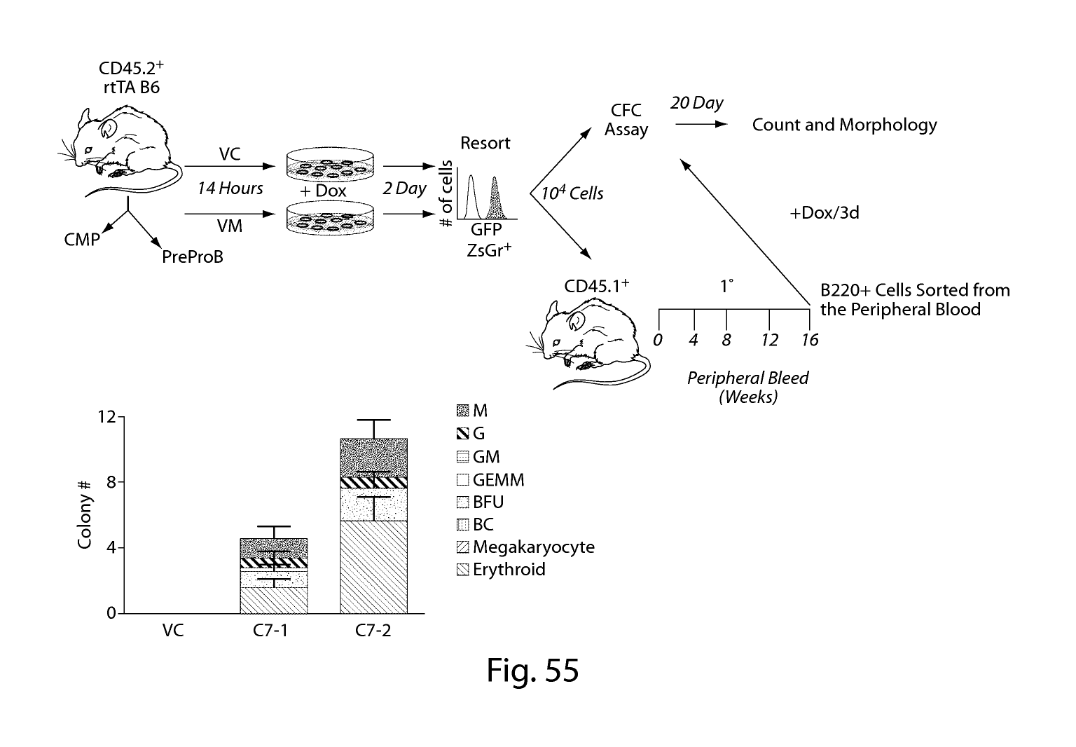

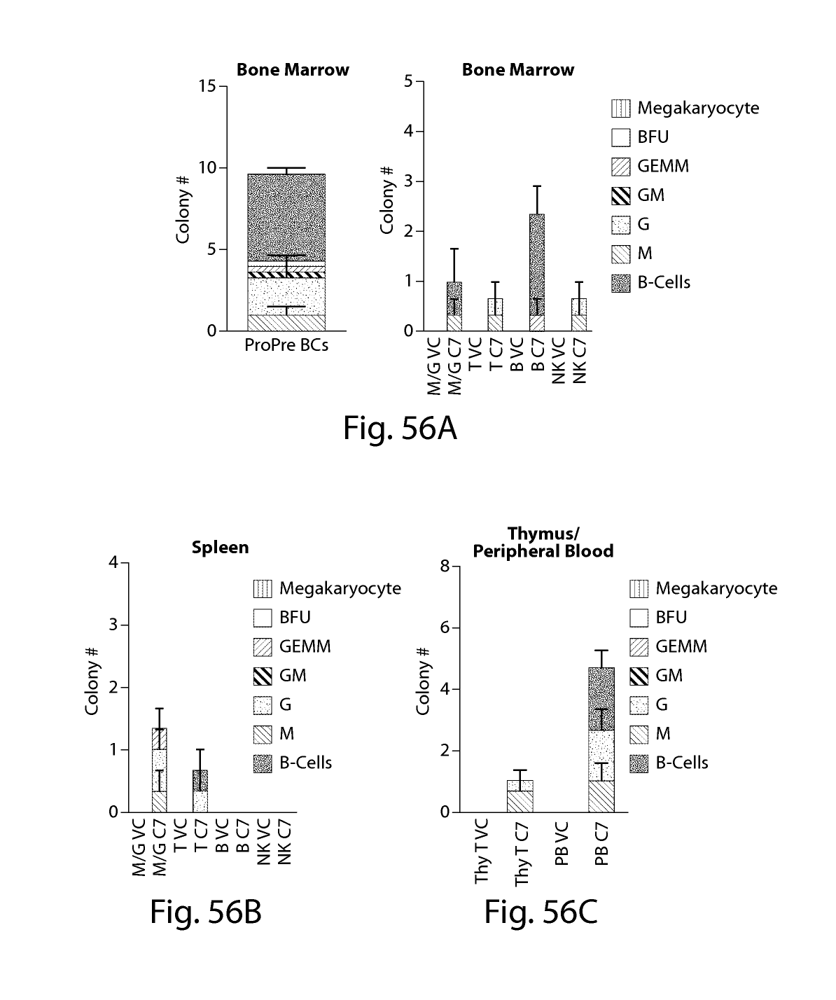

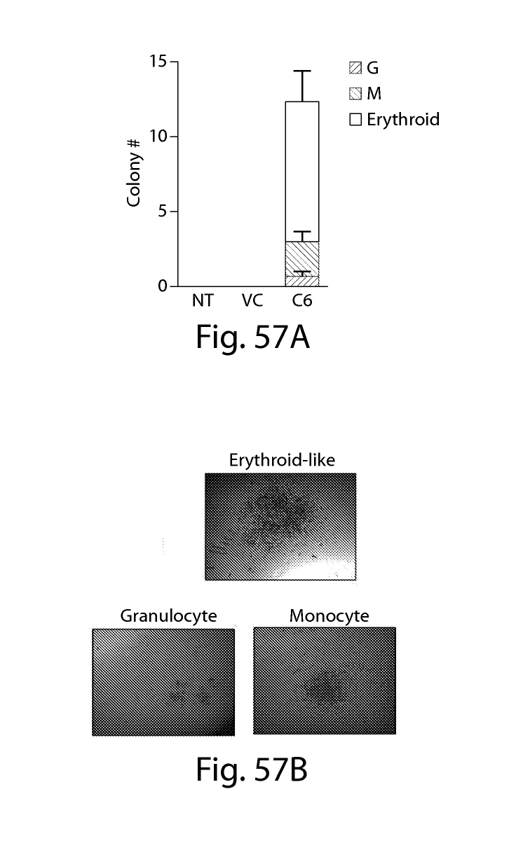

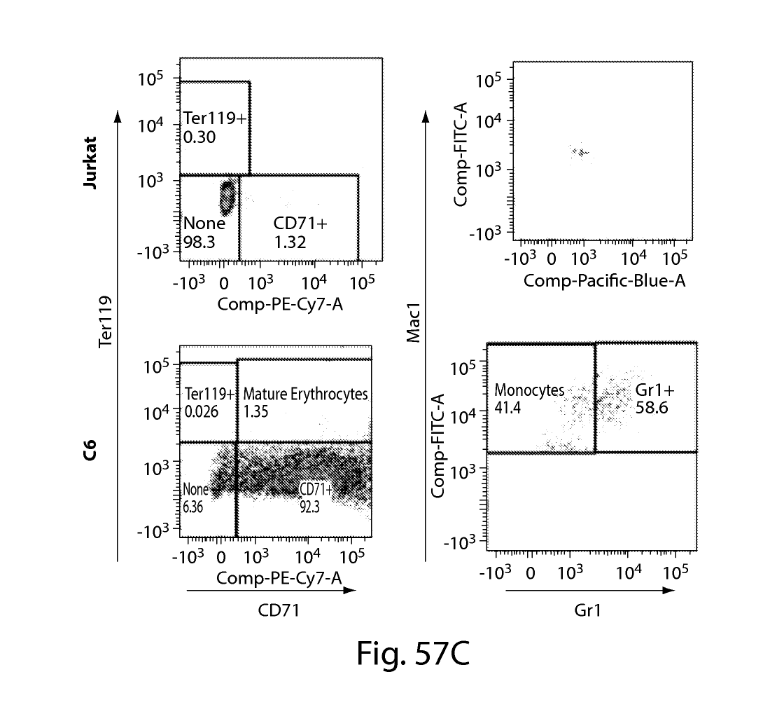

HSCs are most reliably distinguished from committed progenitors by their functional behavior. Functional aspects of HSC phenotypes, or hematopoietic stem cell activities, such as the ability of an HSC to give rise to long-term, multi-lineage reconstitution in a recipient, can be easily determined by one of skill in the art using routine methods known in the art, and as described herein, for example, in the Examples and the Drawings, i.e., FIGS. 1-57C. In some embodiments of the aspects described herein, functional assays to identify reprogramming factors can be used. For example, in some embodiments, Colony forming cell (CFC) activity in methylcellulose can be used to confirm multi-lineage (granulocytes, macrophages, megakaryocytes and erythrocytes) potential of iHSCs generated using the compositions, methods, and kits thereof. Serial plating can be used to confirm self-renewal potential of iHSCs generated using the compositions, methods, and kits described herein. Lymphoid potential of iHSCs generated using the compositions, methods, and kits described herein can be evaluated by culturing transduced cells on OP9 and OP9delta stromal cells, followed by immunostaining on day 14 for B- and T-cells, respectively.

As used herein, “cellular parameter,” “HSC parameter,” or “hematopoietic stem cell activity” refer to measureable components or qualities of endogenous or natural HSCs, particularly components that can be accurately measured. A cellular parameter can be any measurable parameter related to a phenotype, function, or behavior of a cell. Such cellular parameters include, changes in characteristics and markers of an HSC or HSC population, including but not limited to changes in viability, cell growth, expression of one or more or a combination of markers, such as cell surface determinants, such as receptors, proteins, including conformational or posttranslational modification thereof, lipids, carbohydrates, organic or inorganic molecules, nucleic acids, e.g. mRNA, DNA, global gene expression patterns, etc. Such cellular parameters can be measured using any of a variety of assays known to one of skill in the art. For example, viability and cell growth can be measured by assays such as Trypan blue exclusion, CFSE dilution, and ³H incorporation. Expression of protein or polypeptide markers can be measured, for example, using flow cytometric assays, Western blot techniques, or microscopy methods. Gene expression profiles can be assayed, for example, using microarray methodologies and quantitative or semi-quantitative real-time PCR assays. A cellular parameter can also refer to a functional parameter or functional activity. While most cellular parameters will provide a quantitative readout, in some instances a semi-quantitative or qualitative result can be acceptable. Readouts can include a single determined value, or can include mean, median value or the variance, etc. Characteristically a range of parameter readout values can be obtained for each parameter from a multiplicity of the same assays. Variability is expected and a range of values for each of the set of test parameters will be obtained using standard statistical methods with a common statistical method used to provide single values.

In some embodiments of the compositions, methods, and kits described herein, additional factors can be used to enhance HSC reprogramming. For example, agents that modify epigenetic pathways can be used to facilitate reprogramming into iHSCs.

Essentially any primary somatic cell type can be used for producing iHSCs or reprogramming somatic cells to iHSCs according to the presently described compositions, methods, and kits. Such primary somatic cell types also include other stem cell types, including pluripotent stem cells, such as induced pluripotent stem cells (iPS cells); other multipotent stem cells; oligopotent stem cells; and (5) unipotent stem cells. Some non-limiting examples of primary somatic cells useful in the various aspects and embodiments of the methods described herein include, but are not limited to, fibroblast, epithelial, endothelial, neuronal, adipose, cardiac, skeletal muscle, hematopoietic or immune cells, hepatic, splenic, lung, circulating blood cells, gastrointestinal, renal, bone marrow, and pancreatic cells, as well as stem cells from which those cells are derived. The cell can be a primary cell isolated from any somatic tissue including, but not limited to, spleen, bone marrow, blood, brain, liver, lung, gut, stomach, intestine, fat, muscle, uterus, skin, spleen, endocrine organ, bone, etc. The term “somatic cell” further encompasses, in some embodiments, primary cells grown in culture, provided that the somatic cells are not immortalized. Where the cell is maintained under in vitro conditions, conventional tissue culture conditions and methods can be used, and are known to those of skill in the art. Isolation and culture methods for various primary somatic cells are well within the abilities of one skilled in the art.

In some embodiments of the compositions, methods, and kits described herein, a somatic cell to be reprogrammed or made into an iHSC cell is a cell of hematopoietic origin. As used herein, the terms “hematopoietic-derived cell,” “hematopoietic-derived differentiated cell,” “hematopoietic lineage cell,” and “cell of hematopoietic origin” refer to cells derived or differentiated from a multipotent hematopoietic stem cell (HSC). Accordingly, hematopoietic lineage cells for use with the compositions, methods, and kits described herein include multipotent, oligopotent, and lineage-restricted hematopoietic progenitor cells, granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, and lymphocytes (e.g., T-lymphocytes, which carry T-cell receptors (TCRs), B-lymphocytes or B cells, which express immunoglobulin and produce antibodies, NK cells, NKT cells, and innate lymphocytes). As used herein, the term “hematopoietic progenitor cells” refer to multipotent, oligopotent, and lineage-restricted hematopoietic cells capable of differentiating into two or more cell types of the hematopoietic system, including, but not limited to, granulocytes, monocytes, erythrocytes, megakaryocytes, and lymphocytes B-cells and T-cells. Hematopoietic progenitor cells encompass multi-potent progenitor cells (MPPs), common myeloid progenitor cells (CMPs), common lymphoid progenitor cells (CLPs), granulocyte-monocyte progenitor cells (GMPs), and pre-megakaryocyte-erythrocyte progenitor cell. Lineage-restricted hematopoieticprogenitor cells include megakaryocyte-erythrocyte progenitor cells (MEP), roB cells, PreB cells, PreProB cells, ProT cells, double-negative T cells, pro-NK cells, pro-dendritic cells (pro-DCs), pre-granulocyte/macrophage cells, granulocyte/macrophage progenitor (GMP) cells, and pro-mast cells (ProMCs). A differentiation chart of the hematopoietic lineage is provided at FIG. 1

Cells of hematopoietic origin for use in the compositions, methods, and kits described herein can be obtained from any source known to comprise these cells, such as fetal tissues, umbilical cord blood, bone marrow, peripheral blood, mobilized peripheral blood, spleen, liver, thymus, lymph, etc. Cells obtained from these sources can be expanded ex vivo using any method acceptable to those skilled in the art prior to use in with the compositions, methods, and kits for making iHCSs described herein. For example, cells can be sorted, fractionated, treated to remove specific cell types, or otherwise manipulated to obtain a population of cells for use in the methods described herein using any procedure acceptable to those skilled in the art. Mononuclear lymphocytes may be collected, for example, by repeated lymphocytophereses using a continuous flow cell separator as described in U.S. Pat. No. 4,690,915, or isolated using an affinity purification step of common lymphoid progenitor cell (CLP)r method, such as flow-cytometry using a cytometer, magnetic separation, using antibody or protein coated beads, affinity chromatography, or solid-support affinity separation where cells are retained on a substrate according to their expression or lack of expression of a specific protein or type of protein, or batch purification using one or more antibodies against one or more surface antigens specifically expressed by the cell type of interest. Cells of hematopoietic origin can also be obtained from peripheral blood. Prior to harvest of the cells from peripheral blood, the subject can be treated with a cytokine, such as e.g., granulocyte-colony stimulating factor, to promote cell migration from the bone marrow to the blood compartment and/or promote activation and/or proliferation of the population of interest. Any method suitable for identifying surface proteins, for example, can be employed to isolate cells of hematopoietic origin from a heterogenous population. In some embodiments, a clonal population of cells of hematopoietic origin, such as lymphocytes, is obtained. In some embodiments, the cells of hematopoietic origin are not a clonal population.

Further, in regard to the various aspects and embodiments of the compositions, methods, and kits described herein, a somatic cell can be obtained from any mammalian species, with non-limiting examples including a murine, bovine, simian, porcine, equine, ovine, or human cell. In some embodiments, the somatic cell is a human cell. In some embodiments, the cell is from a non-human organism, such as a non-human mammal.

In general, the methods for making iHSCs described herein involve culturing or expanding somatic cells, such as cells of hematopoietic origin, in any culture medium that is available and well-known to one of ordinary skill in the art. Such media include, but are not limited to, Dulbecco's Modified Eagle's Medium® (DMEM), DMEM F12 Medium®, Eagle's Minimum Essential Medium®, F-12K Medium®, Iscove's Modified Dulbecco's Medium®, RPMI-1640 Medium®, and serum-free medium for culture and expansion of progenitor cells SFEM®. Many media are also available as low-glucose formulations, with or without sodium. The medium used with the methods described herein can, in some embodiments, be supplemented with one or more growth factors. Commonly used growth factors include, but are not limited to, bone morphogenic protein, basic fibroblast growth factor, platelet-derived growth factor and epidermal growth factor, Stem cell factor, and thrombopoietin. See, for example, U.S. Pat. Nos. 7,169,610; 7,109,032; 7,037,721; 6,617,161; 6,617,159; 6,372,210; 6,224,860; 6,037,174; 5,908,782; 5,766,951; 5,397,706; and 4,657,866; all incorporated by reference herein in their entireties for teaching growing cells in serum-free medium.

For example, as described herein, primary cultures of mouse hematopoietic cells were kept a total of three days ex vivo during the transduction process. Cells were maintained in minimal growth S-clone media supplemented with 20 ng/μL IL-12, TPO, SCF, 5 ng/μL IL-7, 2 ng/μL FLK-3, and 100 ng/ml Penicillin/streptomycin in a 5% CO₂ 37° C. incubator. Transduction with concentrated and titered viruses was performed for 16 hours, in some embodiments, and then a24 hour incubation with doxycycline, in some embodiments. At this time ZsGr+ cells were re-sorted and put into CFCs assays or in vivo transplantation. Doxycycline induction can be maintained for 2 weeks post-transplant, in some embodiments. In some embodiments, when using an inducible expression vector, the inducing agent, such as doxycycline, can be maintained for at least 1 day, at least 2 days, at least 3 days, at least 4 days, at least 5 days, at least 6 days, at least 7 days or a week, at least 10 days, at least 2 weeks, or more, following transplantation of a induced iHSC population into a subject.

Cells in culture can be maintained either in suspension or attached to a solid support, such as extracellular matrix components or plating on feeder cells, for example. Cells being used in the methods described herein can require additional factors that encourage their attachment to a solid support, in some embodiments, such as type I and type II collagen, chondroitin sulfate, fibronectin, “superfibronectin” and fibronectin-like polymers, gelatin, poly-D and poly-L-lysine, thrombospondin and vitronectin. In some embodiments, the cells are suitable for growth in suspension cultures. Suspension-competent host cells are generally monodisperse or grow in loose aggregates without substantial aggregation. Suspension-competent host cells include cells that are suitable for suspension culture without adaptation or manipulation (e.g., cells of hematopoietic origin, such as lymphoid cells) and cells that have been made suspension-competent by modification or adaptation of attachment-dependent cells (e.g., epithelial cells, fibroblasts).

Also provided herein, in some aspects, are isolated induced hematopoietic stem cells (iHSCs) produced using any of the HSC inducing compositions or methods of preparing iHSCs described herein.

Also provided herein, in some aspects, are cell clones comprising a plurality of the induced hematopoietic stem cell (iHSCs) produced using any of the HSC inducing compositions or methods of preparing iHSCs described herein.

In some embodiments of these aspects and all such aspects described herein, the isolated induced hematopoietic stem cells (iHSCs) or cell clones thereof further comprise a pharmaceutically acceptable carrier for administration to a subject in need.

Also provided herein, in some aspects, are methods of treating a subject in need of treatment for a disease or disorder in which one or more hematopoietic cell lineages are deficient or defective using the HSC inducing compositions and methods of preparing iHSCs described herein, or using the isolated induced hematopoietic stem cells (iHSCs) and cell clones thereof produced using any of the combinations of HSC inducing factors, HSC inducing compositions, or methods of preparing iHSCs described herein. In such methods of treatment, somatic cells, such as fibroblast cells or hematopoietic lineage cells, can first be isolated from the subject, and the isolated cells transduced or transfected, as described herein with an HSC inducing composition comprising expression vectors or synthetic mRNAs, respectively. The isolated induced hematopoietic stem cells (iHSCs) and cell clones thereof produced using any of the combinations of HSC inducing factors, HSC inducing compositions, or methods of preparing iHSCs described herein, can then be administered to the subject, such as via systemic injection of the iHSCs to the subject.

The reprogrammed iHSCs generated using the compositions, methods, and kits described herein can, in some embodiments of the methods of treatment described herein, be used directly or administered to subjects in need of cellular therapies or regenerative medicine applications or, in other embodiments, redifferentiated to other hematopoietic cell types for use in or administration to subjects in need of cellular therapies or regenerative medicine applications. Accordingly, various embodiments of the methods described herein involve administration of an effective amount of an iHSC or a population of iHSCs, generated using any of the compositions, methods, and kits described herein, to an individual or subject in need of a cellular therapy. The cell or population of cells being administered can be an autologous population, or be derived from one or more heterologous sources. Further, such iHSCs or differentiated cells from iHSCs can be administered in a manner that permits them to graft to the intended tissue site and reconstitute or regenerate the functionally deficient area. In some such embodiments, iHSCs can be introduced to a scaffold or other structure to generate, for example, a tissue ex vivo, that can then be introduced to a patient.

A variety of means for administering cells to subjects are known to those of skill in the art. Such methods can include systemic injection, for example, i.v. injection, or implantation of cells into a target site in a subject. Cells may be inserted into a delivery device which facilitates introduction by injection or implantation into the subject. Such delivery devices can include tubes, e.g., catheters, for injecting cells and fluids into the body of a recipient subject. In one preferred embodiment, the tubes additionally have a needle, e.g., through which the cells can be introduced into the subject at a desired location. The cells can be prepared for delivery in a variety of different forms. For example, the cells can be suspended in a solution or gel or embedded in a support matrix when contained in such a delivery device. Cells can be mixed with a pharmaceutically acceptable carrier or diluent in which the cells remain viable.

Accordingly, the cells produced by the methods described herein can be used to prepare cells to treat or alleviate at least the following diseases and conditions wherein hematopoietic stem cell transplants have proven to be one effective method of treatment: leukemia such as acute myeloid leukemia, acute lymphoblastic leukemia, myelodysplastic/myeloproliferative syndromes, chronic myeloid leukemia, chronic lymphocytic leukemia, and other leukemia; lymphoproliferative disorders such as plasma cell disorders, Hodgkin disease, non-Hodgkin lymphoma, and other lymphoma; solid tumors such as neuroblastoma, germinal cancer, breast cancer, and Ewing sarcoma; Nonmalignant disorders such as bone marrow failures, hemoglobinopathies, immune deficiencies, inherited diseases of metabolism, and autoimmune disorders.

In addition to the above, the methods of the invention can be used for the treatment of the following diseases and conditions: Angiogenic Myeloid Metaplasia (Myelofibrosis); Aplastic Anemia; Acquired Pure Red Cell Aplasia; Aspartylglucosaminuria; Ataxia Telangiectasia; Choriocarcinoma; Chronic Lymphocytic Leukemia (CLL); Chronic Myelogenous Leukemia (CML); Common Variable Immunodeficiency; Chronic Pulmonary Obstructive Disease; Desmoplastic small round cell tumor; Diamond-Blackfan anemia; DiGeorge syndrome; Essential Thrombocythemia; Haematologica Ewing's Sarcoma; Fucosidosis; Gaucher disease; Griscelli syndrome; Hemophagocytic lymphohistiocytosis (HLH); Hodgkin's Disease; Human Immunodeficiency Virus (HIV); Human T-lymphotropic Virus (HTLV); Hunter syndrome (MPS II, iduronidase sulfate deficiency); Hurler syndrome (MPS I H, α-L-iduronidase deficiency); Infantile neuronal ceroid lipofuscinosis (INCL, Santavuori disease); Jansky-Bielschowsky disease (late infantile neuronal ceroid lipofuscinosis); Juvenile Myelomonocytic Leukemia (JMML); Kostmann syndrome; Krabbe disease (globoid cell leukodystrophy); Maroteaux-Lamy syndrome (MPS VI); Metachromatic leukodystrophy; Morquio syndrome (MPS IV); Mucolipidosis II (I-cell disease); Multiple Myeloma; Myelodysplasia; Neuroblastoma; NF-Kappa-B Essential Modulator (NEMO) deficiency; Niemann-Pick disease; Non-Hodgkin's Lymphoma; paroxysmal nocturnal hemoglobinuria (PNH); Plasma Cell Leukemia; Polycythemia Vera; Radiation Poisoning; Sanfilippo syndrome (MPS III); Severe combined immunodeficiency (SCID), all types; Shwachman-Diamond syndrome; Sickle cell disease; Sly syndrome (MPS VII); Thalassemia; Wilm's tumors; Wiskott-Aldrich syndrome; Wolman disease (acid lipase deficiency); and X-linked lymphoproliferative disorder

Pharmaceutically acceptable carriers and diluents include saline, aqueous buffer solutions, solvents and/or dispersion media. The use of such carriers and diluents is well known in the art. The solution is preferably sterile and fluid. Preferably, prior to the introduction of cells, the solution is stable under the conditions of manufacture and storage and preserved against the contaminating action of microorganisms such as bacteria and fungi through the use of, for example, parabens, chlorobutanol, phenol, ascorbic acid, thimerosal, and the like.

It is preferred that the mode of cell administration is relatively non-invasive, for example by intravenous injection, pulmonary delivery through inhalation, topical, or intranasal administration. However, the route of cell administration will depend on the tissue to be treated and may include implantation. Methods for cell delivery are known to those of skill in the art and can be extrapolated by one skilled in the art of medicine for use with the methods and compositions described herein.

Direct injection techniques for cellular administration of iHSCs can also be used to stimulate transmigration of cells through the entire vasculature, or to the vasculature of a particular organ. This includes non-specific targeting of the vasculature. One can target any organ by selecting a specific injection site, e.g., a liver portal vein. Alternatively, the injection can be performed systemically into any vein in the body. This method is useful for enhancing stem cell numbers in aging patients. In addition, the cells can function to populate vacant stem cell niches or create new stem cells to replenish those lost through, for example, chemotherapy or radiation treatments, for example. If so desired, a mammal or subject can be pre-treated with an agent, for example an agent is administered to enhance cell targeting to a tissue (e.g., a homing factor) and can be placed at that site to encourage cells to target the desired tissue. For example, direct injection of homing factors into a tissue can be performed prior to systemic delivery of ligand-targeted cells.

A wide range of diseases in which one or more blood cell populations are deficient or defective are recognized as being treatable with HSCs Accordingly, also provided herein are compositions and methods comprising iHSCs for use in cellular therapies, such as stem cell therapies. Non-limiting examples of conditions or disorders that can be treated using the compositions and methods described herein include aplastic anemia, Fanconi anemia, paroxysmal nocturnal hemoglobinuria (PNH); acute leukemias, including acute lymphoblastic leukemia (ALL), acute myelogenous leukemia (AML), acute biphenotypic leukemia and acute undifferentiated leukemia; chronic leukemias, including chronic myelogenous leukemia (CML), chronic lymphocytic leukemia (CLL), juvenile chronic myelogenous leukemia (JCML) and juvenile myelomonocytic leukemia (JMML); myeloproliferative disorders, including acute myelofibrosis, angiogenic myeloid metaplasia (myelofibrosis), polycythemia vera and essential thrombocythemia; inherited platelet abnormalities, including amegakaryocytosis/congenital thrombocytopenia; plasma cell disorders, including multiple myeloma, plasma cell leukemia, and Waldenstrom's macroglobulinemia; lung disorders, including COPD and bronchial asthma; congenital immune disorders, including ataxia-telangiectasia, Kostmann syndrome, leukocyte adhesion deficiency, DiGeorge syndrome, bare lymphocyte syndrome, Omenn's syndrome, severe combined immunodeficiency (SCID), SCID with adenosine deaminase deficiency, absence of T & B cells SCID, absence of T cells, normal B cell SCID, common variable immunodeficiency and X-linked lymphoproliferative disorder, and HIV (human immunodeficiency virus) and AIDS (acquired immune deficiency syndrome).

Efficacy of treatment is determined by a statistically significant change in one or more indicia of the targeted disease or disorder, as known to one of ordinary skill in the art. For example, whole blood of a subject being treated with iHSCs generated using the compositions, methods, and kits described herein can be analyzed using a complete blood count (CBC). A CBC test can comprise one or more of the following:

a. White blood cell (WBC) count: A count of the actual number of white blood cells per volume of blood.
b. White blood cell differential: A count of the types of white blood cells present in the blood: neutrophils, lymphocytes, monocytes, eosinophils, and basophils.
c. Red blood cell (RBC) count: A count of the actual number of red blood cells per volume of blood.
d. Hemoglobin level: A measure of the amount of oxygen-carrying protein in the blood.
e. Hematocrit level: A measures of the percentage of red blood cells in a given volume of whole blood.
f. Platelet count: A count of the number of platelets in a given volume of blood.
g. Mean platelet volume (MPV): A measurement of the average size of platelets. Newly produced platelets are larger and an increased MPV occurs when increased numbers of platelets are being produced in the bone marrow.
h. Mean corpuscular volume (MCV): A measurement of the average size of RBCs (e.g. whether RBCs are larger than normal (macrocytic) or RBCs are smaller than normal (microcytic)).
i. Mean corpuscular hemoglobin (MCH): A calculation of the average amount of oxygen-carrying hemoglobin inside a red blood cell.
j. Mean corpuscular hemoglobin concentration (MCHC): A calculation of the average concentration of hemoglobin inside a red cell (e.g. decreased MCHC values (hypochromia) or increased MCHC values (hyperchromia)),
k. Red cell distribution width (RDW): A calculation of the variation in the size of RBCs {e.g. amount of variation (anisocytosis) in RBC size and/or variation in shape (poikilocytosis) may cause an increase in the RDW).

In some embodiments of the compositions, methods, and kits described herein, additional factors can be used to enhance treatment methods using the iHSCs described herein, such as G-CSF, e.g. as described in U.S. Pat. No. 5,582,823; AMD3100 (1,1[1,4-phenylene-bis(methylene)]-bis-1,4,8,11-tetraazacyclotetradecane), granulocyte-macrophage colony stimulating factor (GM-CSF), Interleukin-1 (IL-I), Interleukin-3 (IL-3), Interleukin-8 (IL-8), PIXY-321 (GM-CSF/IL-3 fusion protein), macrophage inflammatory protein, stem cell factor (SCF), thrombopoietin, flt3, myelopoietin, anti-VLA-4 antibody, anti-VCAM-1 and growth related oncogene (GRO).

Provided herein, in some aspects are hematopoietic stem cell (HSC) inducing composition comprising one or more expression vectors encoding at least one, two, three, four, five, six, seven, eight, or more HSC inducing factors selected from: CDKN1C, DNMT3B, EGR1, ETV6, EVI1, GATA2, GFI1B, GLIS2, HLF, HMGA2, HOXA5, HOXA9, HOXB3, HOXB4, HOXB5, IGF2BP2, IKZF2, KLF12, KLF4, KLF9, LMO2, MEIS1, MSI2, MYCN, NAP1L3, NDN, NFIX, NKX2-3, NR3C2, PBX1, PRDM16, PRDM5, RARB, RBBP6, RBPMS, RUNX1, RUNX1T1, SMAD6, TAL1, TCF15, VDR, ZFP37, ZFP467, ZFP521, ZFP532, and ZFP612.

In some embodiments of these aspects and all such aspects described herein, the at least one, two, three, four, or more HSC inducing factors are HLF, RUNX1T1, PBX1, LMO2, PRDM5, ZFP37, MYCN, MSI2, NKX2-3, MEIS1, and RBPMS.

In some embodiments of these aspects and all such aspects described herein, the at least one, two, three, four, or more HSC inducing factors are HLF, RUNX1T1, PBX1, LMO2, PRDM5, ZFP37, MYCN, and MEIS1.

In some embodiments of these aspects and all such aspects described herein, the at least one, two, three, four, or more HSC inducing factors are HLF, RUNX1T1, ZFP37, PBX1, LMO2, and PRDM5.

In some embodiments of these aspects and all such aspects described herein, the at least one, two, three, four, or more HSC inducing factors are HLF, RUNX1T1, ZFP37, PBX1, and LMO2.

Also provided herein, in some aspects, are hematopoietic stem cell (HSC) inducing compositions comprising one or more expression vectors comprising:

a nucleic acid sequence encoding HLF;

a nucleic acid sequence encoding RUNX1T1;

a nucleic acid sequence encoding ZFP37;

a nucleic acid sequence encoding PBX1;

a nucleic acid sequence encoding LMO2; and

a nucleic acid sequence encoding PRDM5.

Provided herein, in some aspects, are hematopoietic stem cell (HSC) inducing compositions comprising one or more expression vectors comprising:

a nucleic acid sequence encoding HLF;

a nucleic acid sequence encoding RUNX1T1;

a nucleic acid sequence encoding ZFP37;

a nucleic acid sequence encoding PBX1;

a nucleic acid sequence encoding LMO2;

a nucleic acid sequence encoding PRDM5;

a nucleic acid sequence encoding MYCN; and

a nucleic acid sequence encoding MEIS1.

Provided herein, in some aspects, are hematopoietic stem cell (HSC) inducing compositions comprising one or more expression vectors comprising:

a nucleic acid sequence encoding HLF;

a nucleic acid sequence encoding RUNX1T1;

a nucleic acid sequence encoding ZFP37;

a nucleic acid sequence encoding PBX1; and

a nucleic acid sequence encoding LMO2;

In some embodiments of these aspects and all such aspects described herein, the composition further comprises one or more expression vectors comprising:

a nucleic acid sequence encoding PRDM16;

a nucleic acid sequence encoding ZFP467; and

a nucleic acid sequence encoding VDR.

Provided herein, in some aspects, are hematopoietic stem cell (HSC) inducing compositions comprising one or more expression vectors comprising:

a nucleic acid sequence encoding HLF;

a nucleic acid sequence encoding RUNX1T1;

a nucleic acid sequence encoding PBX1;

a nucleic acid sequence encoding LMO2;

a nucleic acid sequence encoding PRDM5

a nucleic acid sequence encoding ZFP37;

a nucleic acid sequence encoding MYCN;

a nucleic acid sequence encoding MSI2;

a nucleic acid sequence encoding NKX2-3;

a nucleic acid sequence encoding MEIS1; and

a nucleic acid sequence encoding RBPMS.

Provided herein, in some aspects, are hematopoietic stem cell (HSC) inducing compositions comprising one or more expression vectors comprising:

a nucleic acid sequence encoding ZFP467;

a nucleic acid sequence encoding PBX1;

a nucleic acid sequence encoding HOXB4; and

a nucleic acid sequence encoding MSI2.

In some embodiments of these aspects and all such aspects described herein, the composition further comprises one or more expression vectors comprising:

a nucleic acid sequence encoding HLF;

a nucleic acid sequence encoding LMO2;

a nucleic acid sequence encoding PRDM16; and

a nucleic acid sequence encoding ZFP37.

Provided herein, in some aspects, are hematopoietic stem cell (HSC) inducing compositions comprising one or more expression vectors comprising:

a nucleic acid sequence encoding MYCN;

a nucleic acid sequence encoding MSI2;

a nucleic acid sequence encoding NKX2-3; and

a nucleic acid sequence encoding RUNX1T1.

In some embodiments of these aspects and all such aspects described herein, the composition further comprises one or more expression vectors comprising:

a nucleic acid sequence encoding HOXB5;

a nucleic acid sequence encoding HLF;

a nucleic acid sequence encoding ZFP467;

a nucleic acid sequence encoding HOXB3;

a nucleic acid sequence encoding LMO2;

a nucleic acid sequence encoding PBX1;

a nucleic acid sequence encoding ZFP37; and

a nucleic acid sequence encoding ZFP521.

Provided herein, in some aspects, are hematopoietic stem cell (HSC) inducing compositions comprising one or more expression vectors comprising:

a nucleic acid sequence encoding HOXB4;

a nucleic acid sequence encoding PBX1;

a nucleic acid sequence encoding LMO2;

a nucleic acid sequence encoding ZFP467; and

a nucleic acid sequence encoding ZFP521.

In some embodiments of these aspects and all such aspects described herein, the composition further comprises one or more expression vectors comprising:

a nucleic acid sequence encoding KLF12;

a nucleic acid sequence encoding HLF; and

a nucleic acid sequence encoding EGR1.

Provided herein, in some aspects, are hematopoietic stem cell (HSC) inducing compositions comprising one or more expression vectors comprising:

a nucleic acid sequence encoding MEIS1;

a nucleic acid sequence encoding RBPMS;

a nucleic acid sequence encoding ZFP37;

a nucleic acid sequence encoding RUNX1T1; and

a nucleic acid sequence encoding LMO2.

In some embodiments of these aspects and all such aspects described herein, the composition further comprises one or more expression vectors comprising:

a sequence encoding KLF12; and

a sequence encoding HLF;

Provided herein, in some aspects, are hematopoietic stem cell (HSC) inducing compositions comprising one or more expression vectors comprising:

a nucleic acid sequence encoding ZFP37;

a nucleic acid sequence encoding HOXB4;

a nucleic acid sequence encoding LMO2; and

a nucleic acid sequence encoding HLF.

In some embodiments of these aspects and all such aspects described herein, the composition further comprises one or more expression vectors comprising:

a nucleic acid sequence encoding MYCN;

a nucleic acid sequence encoding ZFP467;

a nucleic acid sequence encoding NKX2-3

a nucleic acid sequence encoding PBX1; and

a nucleic acid sequence encoding KLF4.

In some embodiments of these aspects and all such aspects described herein, the one or more expression vectors are retroviral vectors.

In some embodiments of these aspects and all such aspects described herein, the one or more expression vectors are lentiviral vectors. In some embodiments, the lentiviral vectors are inducible lentiviral vectors. In some embodiments, the lentiviral vectors are polycistronic inducible lentiviral vectors. In some embodiments, the polycistronic inducible lentiviral vectors express three or more nucleic acid sequences. In some embodiments, each of the nucleic acid sequences of the polycistronic inducible lentiviral vectors are separated by 2A peptide sequences.

Also provided herein, in some aspects, are hematopoietic stem cell (HSC) inducing compositions comprising modified mRNA sequences encoding at least one, two, three, four, five, six, seven, eight, or more HSC inducing factors selected from: CDKN1C, DNMT3B, EGR1, ETV6, EVI1, GATA2, GFI1B, GLIS2, HLF, HMGA2, HOXA5, HOXA9, HOXB3, HOXB4, HOXB5, IGF2BP2, IKZF2, KLF12, KLF4, KLF9, LMO2, MEIS1, MSI2, MYCN, NAP1L3, NDN, NFIX, NKX2-3, NR3C2, PBX1, PRDM16, PRDM5, RARB, RBBP6, RBPMS, RUNX1, RUNX1T1, SMAD6, TAL1, TCF15, VDR, ZFP37, ZFP467, ZFP521, ZFP532, ZFP612, and ZFP467, wherein each cytosine of each said modified mRNA sequence is a modified cytosine, each uracil of each said modified mRNA sequence is a modified uracil, or a combination thereof.

In some embodiments of these aspects and all such aspects described herein, the at least one, two, three, four, or more HSC inducing factors are HLF, RUNX1T1, PBX1, LMO2, PRDM5, ZFP37, MYCN, MSI2, NKX2-3, MEIS1, and RBPMS.

In some embodiments of these aspects and all such aspects described herein, the at least one, two, three, four, or more HSC inducing factors are HLF, RUNX1T1, PBX1, LMO2, PRDM5, ZFP37, MYCN, and MEIS1.

In some embodiments of these aspects and all such aspects described herein, the at least one, two, three, four, or more HSC inducing factors are HLF, RUNX1T1, ZFP37, PBX1, LMO2, and PRDM5.

In some embodiments of these aspects and all such aspects described herein, the at least one, two, three, four, or more HSC inducing factors are HLF, RUNX1T1, ZFP37, PBX1, and LMO2.

Provided herein, in some aspects, are hematopoietic stem cell (HSC) inducing compositions comprising

a modified mRNA sequence encoding HLF;

a modified mRNA sequence encoding RUNX1T1;

a modified mRNA sequence encoding ZFP37;

a modified mRNA sequence encoding PBX1;

a modified mRNA sequence encoding LMO2; and

a modified mRNA sequence encoding PRDM5;

wherein each cytosine of each said modified mRNA sequence is a modified cytosine, each uracil of each said modified mRNA sequence is a modified uracil, or a combination thereof.

Provided herein, in some aspects, are hematopoietic stem cell (HSC) inducing compositions comprising

a modified mRNA sequence encoding HLF;

a modified mRNA sequence encoding RUNX1T1;

a modified mRNA sequence encoding ZFP37;

a modified mRNA sequence encoding PBX1;

a modified mRNA sequence encoding LMO2;

a modified mRNA sequence encoding PRDM5;

a modified mRNA sequence encoding MEIS1; and

a modified mRNA sequence encoding MYCN;

wherein each cytosine of each said modified mRNA sequence is a modified cytosine, each uracil of each said modified mRNA sequence is a modified uracil, or a combination thereof.

Provided herein, in some aspects, are hematopoietic stem cell (HSC) inducing compositions comprising

a modified mRNA sequence encoding HLF;

a modified mRNA sequence encoding RUNX1T1;

a modified mRNA sequence encoding ZFP37;

a modified mRNA sequence encoding PBX1; and

a modified mRNA sequence encoding LMO2;

wherein each cytosine of each said modified mRNA sequence is a modified cytosine, each uracil of each said modified mRNA sequence is a modified uracil, or a combination thereof.

In some embodiments of these aspects and all such aspects described herein, the composition further comprises one or more of:

a modified mRNA sequence encoding PRDM16;

a modified mRNA sequence encoding ZFP467; and

a modified mRNA sequence encoding VDR;

wherein each cytosine of each said modified mRNA sequence is a modified cytosine, each uracil of each said modified mRNA sequence is a modified uracil, or a combination thereof.

Provided herein, in some aspects, are hematopoietic stem cell (HSC) inducing compositions comprising

a modified mRNA sequence encoding HLF;

a modified mRNA sequence encoding RUNX1T1;

a modified mRNA sequence encoding PBX1;

a modified mRNA sequence encoding LMO2;

a modified mRNA sequence encoding PRDM5

a modified mRNA sequence encoding ZFP37;

a modified mRNA sequence encoding MYCN;

a modified mRNA sequence encoding MSI2;

a modified mRNA sequence encoding NKX2-3;

a modified mRNA sequence encoding MEIS1; and

a modified mRNA sequence encoding RBPMS;

wherein each cytosine of each said modified mRNA sequence is a modified cytosine, each uracil of each said modified mRNA sequence is a modified uracil, or a combination thereof.

Provided herein, in some aspects, are hematopoietic stem cell (HSC) inducing compositions comprising

a modified mRNA sequence encoding ZFP467;

a modified mRNA sequence encoding PBX1;

a modified mRNA sequence encoding HOXB4; and

a modified mRNA sequence encoding MSI2;

wherein each cytosine of each said modified mRNA sequence is a modified cytosine, each uracil of each said modified mRNA sequence is a modified uracil, or a combination thereof.

In some embodiments of these aspects and all such aspects described herein, the composition further comprises one or more of:

a modified mRNA sequence encoding HLF;

a modified mRNA sequence encoding LMO2;

a modified mRNA sequence encoding PRDM16; and

a modified mRNA sequence encoding ZFP37.

wherein each cytosine of each said modified mRNA sequence is a modified cytosine, each uracil of each said modified mRNA sequence is a modified uracil, or a combination thereof.

Provided herein, in some aspects, are hematopoietic stem cell (HSC) inducing compositions comprising

a modified mRNA sequence encoding MYCN;

a modified mRNA sequence encoding MSI2;

a modified mRNA sequence encoding NKX2-3; and

a modified mRNA sequence encoding RUNX1T1;

wherein each cytosine of each said modified mRNA sequence is a modified cytosine, each uracil of each said modified mRNA sequence is a modified uracil, or a combination thereof.

In some embodiments of these aspects and all such aspects described herein, the composition further comprises one or more of:

a modified mRNA sequence encoding HOXB5;

a modified mRNA sequence encoding HLF;

a modified mRNA sequence encoding ZFP467;

a modified mRNA sequence encoding HOXB3;

a modified mRNA sequence encoding LMO2;

a modified mRNA sequence encoding PBX1;

a modified mRNA sequence encoding ZFP37; and

a modified mRNA sequence encoding ZFP521;

wherein each cytosine of each said modified mRNA sequence is a modified cytosine, each uracil of each said modified mRNA sequence is a modified uracil, or a combination thereof.

Provided herein, in some aspects, are hematopoietic stem cell (HSC) inducing compositions comprising

a modified mRNA sequence encoding HOXB4;

a modified mRNA sequence encoding PBX1;

a modified mRNA sequence encoding LMO2;

a modified mRNA sequence encoding ZFP467; and

a modified mRNA sequence encoding ZFP521;

wherein each cytosine of each said modified mRNA sequence is a modified cytosine, each uracil of each said modified mRNA sequence is a modified uracil, or a combination thereof.

In some embodiments of these aspects and all such aspects described herein, the composition further comprises one or more of:

a modified mRNA sequence encoding KLF12;

a modified mRNA sequence encoding HLF; and

a modified mRNA sequence encoding EGR;

wherein each cytosine of each said modified mRNA sequence is a modified cytosine, each uracil of each said modified mRNA sequence is a modified uracil, or a combination thereof.

Provided herein, in some aspects, are hematopoietic stem cell (HSC) inducing compositions comprising

a modified mRNA sequence encoding MEIS1;

a modified mRNA sequence encoding RBPMS;

a modified mRNA sequence encoding ZFP37;

a modified mRNA sequence encoding RUNX1T1; and

a modified mRNA sequence encoding LMO2.

wherein each cytosine of each said modified mRNA sequence is a modified cytosine, each uracil of each said modified mRNA sequence is a modified uracil, or a combination thereof.

In some embodiments of these aspects and all such aspects described herein, the composition further comprises one or more of:

a modified mRNA sequence encoding KLF12; and

a modified mRNA sequence encoding HLF;

wherein each cytosine of each said modified mRNA sequence is a modified cytosine, each uracil of each said modified mRNA sequence is a modified uracil, or a combination thereof.

Provided herein, in some aspects, are hematopoietic stem cell (HSC) inducing compositions comprising

a modified mRNA sequence encoding ZFP37;

a modified mRNA sequence encoding HOXB4;

a modified mRNA sequence encoding LMO2; and

a modified mRNA sequence encoding HLF;

wherein each cytosine of each said modified mRNA sequence is a modified cytosine, each uracil of each said modified mRNA sequence is a modified uracil, or a combination thereof.

In some embodiments of these aspects and all such aspects described herein, the composition further comprises one or more of:

a modified mRNA encoding MYCN;

a modified mRNA encoding ZFP467;

a modified mRNA encoding NKX2-3

a modified mRNA encoding PBX1; and

a modified mRNA encoding KLF4;

wherein each cytosine of each said modified mRNA sequence is a modified cytosine, each uracil of each said modified mRNA sequence is a modified uracil, or a combination thereof.

In some embodiments of these aspects and all such aspects described herein, the modified cytosine is 5-methylcytosine and the modified uracil is pseudouracil.

In some embodiments of these aspects and all such aspects described herein, the modified mRNA sequences comprise one or more nucleoside modifications selected from the group consisting of pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, 4-methoxy-2-thio-pseudouridine, 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, 2-aminopurine, 2,6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl)adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine, inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine, and combinations thereof.

Also provided herein in some aspects, are methods for preparing an induced hematopoietic stem cell (iHSC) from a somatic cell comprising:

transducing the somatic cell with one or more vectors comprising a nucleic acid sequence encoding HLF, a nucleic acid sequence encoding RUNX1T1; a nucleic acid sequence encoding ZFP37; a nucleic acid sequence encoding PBX1; a nucleic acid sequence encoding LMO2; and a nucleic acid sequence encoding PRDM5, wherein each said nucleic acid sequence is operably linked to a promoter; and

culturing the transduced somatic cell in a cell media that supports growth of hematopoietic stem cells, thereby preparing an iHSC.

Provided herein in some aspects, are methods for preparing an induced hematopoietic stem cell (iHSC) from a somatic cell comprising:

transducing the somatic cell with one or more vectors comprising a nucleic acid sequence encoding HLF, a nucleic acid sequence encoding RUNX1T1; a nucleic acid sequence encoding ZFP37; a nucleic acid sequence encoding PBX1; a nucleic acid sequence encoding LMO2; and a nucleic acid sequence encoding PRDM5; a nucleic acid sequence encoding MEIS1; and a nucleic acid sequence encoding MYCN, wherein each said nucleic acid sequence is operably linked to a promoter; and

culturing the transduced somatic cell in a cell media that supports growth of hematopoietic stem cells, thereby preparing an iHSC.

Provided herein in some aspects, are methods for preparing an induced hematopoietic stem cell (iHSC) from a somatic cell comprising:

transducing the somatic cell with one or more vectors comprising a nucleic acid sequence encoding HLF; a nucleic acid sequence encoding RUNX1T1; a nucleic acid sequence encoding ZFP37; a nucleic acid sequence encoding PBX1; and a nucleic acid sequence encoding LMO2; and a nucleic acid sequence encoding PRDM5, wherein each said nucleic acid sequence is operably linked to a promoter; and

culturing the transduced somatic cell in a cell media that supports growth of hematopoietic stem cells, thereby preparing an iHSC.

In some embodiments of these aspects and all such aspects described herein, the transducing of step (a) further comprises one or more vectors comprising one or more of: a nucleic acid sequence encoding PRDM16 a nucleic acid sequence encoding ZFP467; and a nucleic acid sequence encoding VDR.

Provided herein in some aspects, are methods for preparing an induced hematopoietic stem cell (iHSC) from a somatic cell comprising:

transducing the somatic cell with one or more vectors comprising a nucleic acid sequence encoding HLF, a nucleic acid sequence encoding RUNX1T1; a nucleic acid sequence encoding PBX1; a nucleic acid sequence encoding LMO2; a nucleic acid sequence encoding PRDM5; a nucleic acid sequence encoding ZFP37; a nucleic acid sequence encoding MYCN; a nucleic acid sequence encoding MSI2; a nucleic acid sequence encoding NKX2-3; a nucleic acid sequence encoding MEIS1; and a nucleic acid sequence encoding RBPMS; wherein each said nucleic acid sequence is operably linked to a promoter; and

culturing the transduced somatic cell in a cell media that supports growth of hematopoietic stem cells, thereby preparing an iHSC.

Provided herein in some aspects, are methods for preparing an induced hematopoietic stem cell (iHSC) from a somatic cell comprising:

transducing the somatic cell with one or more vectors comprising a nucleic acid sequence encoding ZFP467, a nucleic acid sequence encoding PBX1; a nucleic acid sequence encoding HOXB4; and a nucleic acid sequence encoding MSI2; wherein each said nucleic acid sequence is operably linked to a promoter; and

culturing the transduced somatic cell in a cell media that supports growth of hematopoietic stem cells, thereby preparing an iHSC.

In some embodiments of these aspects and all such aspects described herein, the transducing of step (a) further comprises one or more vectors comprising one or more of: a nucleic acid sequence encoding HLF, a nucleic acid sequence encoding LMO2; a nucleic acid sequence encoding PRDM16; and a nucleic acid sequence encoding ZFP37.

Provided herein in some aspects, are methods for preparing an induced hematopoietic stem cell (iHSC) from a somatic cell comprising:

transducing the somatic cell with one or more vectors comprising a nucleic acid sequence encoding MYCN; a nucleic acid sequence encoding MSI2, a nucleic acid sequence encoding NKX2-3; and a nucleic acid sequence encoding RUNX1T1; wherein each said nucleic acid sequence is operably linked to a promoter; and

culturing the transduced somatic cell in a cell media that supports growth of hematopoietic stem cells, thereby preparing an iHSC.

In some embodiments of these aspects and all such aspects described herein, the transducing of step (a) further comprises one or more vectors comprising one or more of: a nucleic acid sequence encoding HOXB5; a nucleic acid sequence encoding HLF, a nucleic acid sequence encoding ZFP467; a nucleic acid sequence encoding HOXB3; a nucleic acid sequence encoding LMO2; a nucleic acid sequence encoding PBX1; a nucleic acid sequence encoding ZFP37; and a nucleic acid sequence encoding ZFP521.

Provided herein in some aspects, are methods for preparing an induced hematopoietic stem cell (iHSC) from a somatic cell comprising:

transducing the somatic cell with one or more vectors comprising a nucleic acid sequence encoding HOXB4; a nucleic acid sequence encoding PBX1, a nucleic acid sequence encoding LMO2; a nucleic acid sequence encoding ZFP467; and a nucleic acid sequence encoding ZFP521; wherein each said nucleic acid sequence is operably linked to a promoter; and

culturing the transduced somatic cell in a cell media that supports growth of hematopoietic stem cells, thereby preparing an iHSC.

In some embodiments of these aspects and all such aspects described herein, the transducing of step (a) further comprises one or more vectors comprising one or more of: a nucleic acid sequence encoding KLF12; a nucleic acid sequence encoding HLF; and a nucleic acid sequence encoding EGR1.

Provided herein, in some aspects, are methods for preparing an induced hematopoietic stem cell (iHSC) from a somatic cell comprising:

transducing the somatic cell with one or more vectors comprising a nucleic acid sequence encoding MEIS1; a nucleic acid sequence encoding RBPMS; a nucleic acid sequence encoding ZFP37; a nucleic acid sequence encoding RUNX1T1; and a nucleic acid sequence encoding LMO2; wherein each said nucleic acid sequence is operably linked to a promoter; and

culturing the transduced somatic cell in a cell media that supports growth of hematopoietic stem cells, thereby preparing an iHSC.

In some embodiments of these aspects and all such aspects described herein, the transducing of step (a) further comprises one or more vectors comprising one or more of: a nucleic acid sequence encoding KLF12; and a nucleic acid sequence encoding HLF.

Provided herein, in some aspects, are methods for preparing an induced hematopoietic stem cell (iHSC) from a somatic cell comprising:

transducing the somatic cell with one or more vectors comprising a nucleic acid sequence encoding ZFP37; a nucleic acid sequence encoding HOXB4; a nucleic acid sequence encoding LMO2; and a nucleic acid sequence encoding HLF; wherein each said nucleic acid sequence is operably linked to a promoter; and

culturing the transduced somatic cell in a cell media that supports growth of hematopoietic stem cells, thereby preparing an iHSC.

In some embodiments of these aspects and all such aspects described herein, the transducing of step (a) further comprises one or more vectors comprising one or more of: a nucleic acid sequence encoding KLF12; and a nucleic acid sequence encoding HLF.

Provided herein, in some aspects, are methods for preparing an induced hematopoietic stem cell (iHSC) from a somatic cell comprising:

transducing the somatic cell with one or more vectors comprising a nucleic acid sequence encoding ZFP37; a nucleic acid sequence encoding HOXB4; a nucleic acid sequence encoding LMO2; and a nucleic acid sequence encoding HLF; wherein each said nucleic acid sequence is operably linked to a promoter; and

culturing the transduced somatic cell in a cell media that supports growth of hematopoietic stem cells, thereby preparing an iHSC.

In some embodiments of these aspects and all such aspects described herein, the transducing of step (a) further comprises one or more vectors comprising one or more of: a nucleic acid sequence encoding MYCN; a nucleic acid sequence encoding ZFP467; a nucleic acid sequence encoding NKX2-3; a nucleic acid sequence encoding PBX1; and a nucleic acid sequence encoding KLF4.

In some embodiments of these aspects and all such aspects described herein, the somatic cell is a fibroblast cell.

In some embodiments of these aspects and all such aspects described herein, the somatic cell is a hematopoietic lineage cell.

In some embodiments of these aspects and all such aspects described herein, the hematopoietic lineage cell is selected from promyelocytes, neutrophils, eosinophils, basophils, reticulocytes, erythrocytes, mast cells, osteoclasts, megakaryoblasts, platelet producing megakaryocytes, platelets, monocytes, macrophages, dendritic cells, lymphocytes, NK cells, NKT cells, innate lymphocytes, multipotent hematopoietic progenitor cells, oligopotent hematopoietic progenitor cells, and lineage restricted hematopoietic progenitors.

In some embodiments of these aspects and all such aspects described herein, the hematopoietic lineage cell is selected from a multi-potent progenitor cell (MPP), common myeloid progenitor cell (CMP), granulocyte-monocyte progenitor cells (GMP), common lymphoid progenitor cell (CLP), and pre-megakaryocyte-erythrocyte progenitor cell.

In some embodiments of these aspects and all such aspects described herein, the hematopoietic lineage cell is selected from a megakaryocyte-erythrocyte progenitor cell (MEP), a ProB cell, a PreB cell, a PreProB cell, a ProT cell, a double-negative T cell, a pro-NK cell, a pro-dendritic cell (pro-DC), pre-granulocyte/macrophage cell, a granulocyte/macrophage progenitor (GMP) cell, and a pro-mast cell (ProMC).

Also provided herein, in some aspects, are methods of promoting transdifferentiation of a ProPreB cell to the myeloid lineage comprising:

transducing a ProPreB cell with one or more vectors comprising a nucleic acid sequence encoding ZFP467, a nucleic acid sequence encoding PBX1; a nucleic acid sequence encoding HOXB4; and a nucleic acid sequence encoding MSI2; wherein each said nucleic acid sequence is operably linked to a promoter; and

culturing the transduced ProPreB cell in a cell media that supports growth of myeloid lineage cells, thereby transdifferentiating the ProPreB cell to the myeloid lineage.

In some embodiments of these aspects and all such aspects described herein, the transducing of step (a) further comprises one or more vectors comprising one or more of: a nucleic acid sequence encoding HLF, a nucleic acid sequence encoding LMO2; a nucleic acid sequence encoding PRDM16; and a nucleic acid sequence encoding ZFP37.

Also provided herein, in some aspects, are methods of increasing survival and/or proliferation of ProPreB cells, comprising:

transducing a ProPreB cell with one or more vectors comprising a nucleic acid sequence encoding HOXB4; a nucleic acid sequence encoding PBX1, a nucleic acid sequence encoding LMO2; a nucleic acid sequence encoding ZFP467; and a nucleic acid sequence encoding ZFP521; wherein each said nucleic acid sequence is operably linked to a promoter; and

culturing the transduced ProPreB cell in a cell media that supports growth of ProPreB cells, thereby increasing survival and/or proliferation of ProPreB cells.

In some embodiments of these aspects and all such aspects described herein, the transducing of step (a) further comprises one or more vectors comprising one or more of: a nucleic acid sequence encoding KLF12; a nucleic acid sequence encoding HLF; and a nucleic acid sequence encoding EGR1.

Also provided herein, in some aspects, are isolated induced hematopoietic stem cells (iHSCs) produced using any of the HSC inducing compositions or methods described herein.

In some aspects, provided herein are cell clones comprising a plurality of the induced hematopoietic stem cells (iHSCs) produced using any of the HSC inducing compositions or methods described herein. In some embodiments of these aspects and all such aspects described herein, the cell clones further comprise a pharmaceutically acceptable carrier.

Also provided herein, in some aspects, are kits for making induced hematopoietic stem cells (iHSCs), the kits comprising any of the HSC inducing compositions comprising one or more expression vector components described herein.

Provided herein, in some aspects, are kits for making induced hematopoietic stem cells (iHSCs), the kits comprising any of the HSC inducing compositions comprising modified mRNA sequence components described herein.

Also provided herein, in some aspects, are kits comprising one or more of the HSC inducing factors described herein as components for the methods of making the induced hematopoietic stem cells described herein.

Accordingly, in some aspects, provided herein, are kits for preparing induced hematopoietic stem cells comprising the following components: (a) one or more expression vectors encoding at least one, two, three, four, five, six, seven, eight, or more HSC inducing factors selected from: CDKN1C, DNMT3B, EGR1, ETV6, EVI1, GATA2, GFI1B, GLIS2, HLF, HMGA2, HOXA5, HOXA9, HOXB3, HOXB4, HOXB5, IGF2BP2, IKZF2, KLF12, KLF4, KLF9, LMO2, MEIS1, MSI2, MYCN, NAP1L3, NDN, NFIX, NKX2-3, NR3C2, PBX1, PRDM16, PRDM5, RARB, RBBP6, RBPMS, RUNX1, RUNX1T1, SMAD6, TAL1, TCF15, VDR, ZFP37, ZFP467, ZFP521, ZFP532, ZFP612, and ZFP467; and (b) packaging and instructions therefor.

In some embodiments of these kits and all such kits described herein, the at least one, two, three, four, or more HSC inducing factors are HLF, RUNX1T1, PBX1, LMO2, PRDM5, ZFP37, MYCN, MSI2, NKX2-3, MEIS1, and RBPMS.

In some embodiments of these kits and all such kits described herein, the at least one, two, three, four, or more HSC inducing factors are HLF, RUNX1T1, ZFP37, PBX1, LMO2, and PRDM5.

In some aspects, provided herein, are kits for preparing induced hematopoietic stem cells comprising the following components: (a) one or more expression vectors comprising: a nucleic acid sequence encoding HLF; a nucleic acid sequence encoding RUNX1T1; a nucleic acid sequence encoding ZFP37; a nucleic acid sequence encoding PBX1; a nucleic acid sequence encoding LMO2; and a nucleic acid sequence encoding PRDM5; and (b) packaging and instructions therefor.

In some embodiments of these kits and all such kits described herein, the kit further comprises one or more of: a nucleic acid sequence encoding PRDM16; a nucleic acid sequence encoding ZFP467; and a nucleic acid sequence encoding VDR.

In some aspects, provided herein, are kits for preparing induced hematopoietic stem cells comprising the following components: (a) one or more expression vectors comprising: a nucleic acid sequence encoding HLF; a nucleic acid sequence encoding RUNX1T1; a nucleic acid sequence encoding PBX1; a nucleic acid sequence encoding LMO2; a nucleic acid sequence encoding PRDM5; a nucleic acid sequence encoding ZFP37; a nucleic acid sequence encoding MYCN; a nucleic acid sequence encoding MSI2; a nucleic acid sequence encoding NKX2-3; a nucleic acid sequence encoding MEIS1; and a nucleic acid sequence encoding RBPMS; and (b) packaging and instructions therefor.

In some aspects, provided herein, are kits for preparing induced hematopoietic stem cells comprising the following components: (a) one or more expression vectors comprising: a nucleic acid sequence encoding ZFP467; a nucleic acid sequence encoding PBX1; a nucleic acid sequence encoding HOXB4; and a nucleic acid sequence encoding MSI2; and (b) packaging and instructions therefor.

In some embodiments of these kits and all such kits described herein, the kit further comprises one or more of: a nucleic acid sequence encoding HLF; a nucleic acid sequence encoding LMO2; a nucleic acid sequence encoding PRDM16; and a nucleic acid sequence encoding ZFP37.

In some aspects, provided herein, are kits for preparing induced hematopoietic stem cells comprising the following components: (a) one or more expression vectors comprising: a nucleic acid sequence encoding MYCN; a nucleic acid sequence encoding MSI2; a nucleic acid sequence encoding NKX2-3; and a nucleic acid sequence encoding RUNX1T1; and (b) packaging and instructions therefor.

In some embodiments of these kits and all such kits described herein, the kit further comprises a nucleic acid sequence encoding HOXB5; a nucleic acid sequence encoding HLF; a nucleic acid sequence encoding ZFP467; a nucleic acid sequence encoding HOXB3; a nucleic acid sequence encoding LMO2; a nucleic acid sequence encoding PBX1; a nucleic acid sequence encoding ZFP37; and a nucleic acid sequence encoding ZFP521.

In some aspects, provided herein, are kits for preparing induced hematopoietic stem cells comprising the following components: (a) one or more expression vectors composition comprising: a nucleic acid sequence encoding HOXB4; a nucleic acid sequence encoding PBX1; a nucleic acid sequence encoding LMO2; a nucleic acid sequence encoding ZFP467; and a nucleic acid sequence encoding ZFP521; and (b) packaging and instructions therefor.

In some embodiments of these kits and all such kits described herein, the kit further comprises one or more of: a nucleic acid sequence encoding KLF12; a nucleic acid sequence encoding HLF; and a nucleic acid sequence encoding EGR1.

In some aspects, provided herein, are kits for preparing induced hematopoietic stem cells comprising the following components: (a) one or more expression vectors comprising: a nucleic acid sequence encoding MEIS1; a nucleic acid sequence encoding RBPMS; a nucleic acid sequence encoding ZFP37; a nucleic acid sequence encoding RUNX1T1; and a nucleic acid sequence encoding LMO2; and (b) packaging and instructions therefor.

In some embodiments of these kits and all such kits described herein, the kit further comprises one or more of a sequence encoding KLF12; and a sequence encoding HLF.

In some aspects, provided herein, are kits for preparing induced hematopoietic stem cells comprising the following components: (a) one or more expression vectors comprising: a nucleic acid sequence encoding ZFP37; a nucleic acid sequence encoding HOXB4; a nucleic acid sequence encoding LMO2; and a nucleic acid sequence encoding HLF; and (b) packaging and instructions therefor.

In some embodiments of these kits and all such kits described herein, the kit further comprises one or more of: a nucleic acid sequence encoding MYCN; a nucleic acid sequence encoding ZFP467; a nucleic acid sequence encoding NKX2-3; a nucleic acid sequence encoding PBX1; and a nucleic acid sequence encoding KLF4.

In some embodiments of these kits, the expression vector is a viral vector. In some embodiments of these kits, the viral vector is a retroviral vector, adenoviral vector, lentiviral vector, herpes virus vector, pox virus vector, or an adeno-associated virus (AAV) vector. In some embodiments, the expression vector is inducible.

Also provided herein, in some aspects, are kits for preparing induced hematopoietic stem cells comprising the following components: (a) modified mRNA sequences encoding at least one, two, three, four, five, six, seven, eight, or more HSC inducing factors selected from: CDKN1C, DNMT3B, EGR1, ETV6, EVI1, GATA2, GFI1B, GLIS2, HLF, HMGA2, HOXA5, HOXA9, HOXB3, HOXB4, HOXB5, IGF2BP2, IKZF2, KLF12, KLF4, KLF9, LMO2, MEIS1, MSI2, MYCN, NAP1L3, NDN, NFIX, NKX2-3, NR3C2, PBX1, PRDM16, PRDM5, RARB, RBBP6, RBPMS, RUNX1, RUNX1T1, SMAD6, TAL1, TCF15, VDR, ZFP37, ZFP467, ZFP521, ZFP532, and ZFP612, wherein each cytosine of each of the modified mRNA sequences is a modified cytosine, each uracil of each of the modified mRNA sequences is a modified uracil, or a combination thereof.

In some embodiments of these kits and all such kits described herein, the at least one, two, three, four, or more HSC inducing factors are HLF, RUNX1T1, PBX1, LMO2, PRDM5, ZFP37, MYCN, MSI2, NKX2-3, MEIS1, and RBPMS.

In some embodiments of these kits and all such kits described herein, the at least one, two, three, four, or more HSC inducing factors are HLF, RUNX1T1, ZFP37, PBX1, LMO2, and PRDM5

In some aspects, provided herein, are kits for preparing induced hematopoietic stem cells comprising the following components: (a) a modified mRNA sequence encoding HLF; a modified mRNA sequence encoding RUNX1T1; a modified mRNA sequence encoding ZFP37; a modified mRNA sequence encoding PBX1; a modified mRNA sequence encoding LMO2; and a modified mRNA sequence encoding PRDM5; wherein each cytosine of each of the modified mRNA sequences is a modified cytosine, each uracil of each of the modified mRNA sequences is a modified uracil, or a combination thereof and (b) packaging and instructions therefor.

In some embodiments of these kits and all such kits described herein, the kit further comprises one or more of: a modified mRNA sequence encoding PRDM16; a modified mRNA sequence encoding ZFP467; and a modified mRNA sequence encoding VDR; wherein each cytosine of each of the modified mRNA sequences is a modified cytosine, each uracil of each of the modified mRNA sequences is a modified uracil, or a combination thereof.

In some aspects, provided herein, are kits for preparing induced hematopoietic stem cells comprising the following components: (a) a modified mRNA sequence encoding HLF; a modified mRNA sequence encoding RUNX1T1; a modified mRNA sequence encoding PBX1; a modified mRNA sequence encoding LMO2; a modified mRNA sequence encoding PRDM5; a modified mRNA sequence encoding ZFP37; a modified mRNA sequence encoding MYCN; a modified mRNA sequence encoding MSI2; a modified mRNA sequence encoding NKX2-3; a modified mRNA sequence encoding MEIS1; and a modified mRNA sequence encoding RBPMS; wherein each cytosine of each of the modified mRNA sequences is a modified cytosine, each uracil of each of the modified mRNA sequences is a modified uracil, or a combination thereof and (b) packaging and instructions therefor.

In some aspects, provided herein, are kits for preparing induced hematopoietic stem cells comprising the following components: (a) a modified mRNA sequence encoding ZFP467; a modified mRNA sequence encoding PBX1; a modified mRNA sequence encoding HOXB4; and a modified mRNA sequence encoding MSI2; wherein each cytosine of each of the modified mRNA sequences is a modified cytosine, each uracil of each of the modified mRNA sequences is a modified uracil, or a combination thereof; and (b) packaging and instructions therefor.

In some embodiments of these kits and all such kits described herein, the kit further comprises one or more of: a modified mRNA sequence encoding HLF; a modified mRNA sequence encoding LMO2; a modified mRNA sequence encoding PRDM16; and a modified mRNA sequence encoding ZFP37, wherein each cytosine of each of the modified mRNA sequences is a modified cytosine, each uracil of each of the modified mRNA sequences is a modified uracil, or a combination thereof.

In some aspects, provided herein, are kits for preparing induced hematopoietic stem cells comprising the following components: (a) a modified mRNA sequence encoding MYCN; a modified mRNA sequence encoding MSI2; a modified mRNA sequence encoding NKX2-3; and a modified mRNA sequence encoding RUNX1T1; wherein each cytosine of each of the modified mRNA sequences is a modified cytosine, each uracil of each of the modified mRNA sequences is a modified uracil, or a combination thereof; and (b) packaging and instructions therefor.

In some embodiments of these kits and all such kits described herein, the kit further comprises one or more of: a modified mRNA sequence encoding HOXB5; a modified mRNA sequence encoding HLF; a modified mRNA sequence encoding ZFP467; a modified mRNA sequence encoding HOXB3; a modified mRNA sequence encoding LMO2; a modified mRNA sequence encoding PBX1; a modified mRNA sequence encoding ZFP37; and a modified mRNA sequence encoding ZFP521; wherein each cytosine of each of the modified mRNA sequences is a modified cytosine, each uracil of each of the modified mRNA sequences is a modified uracil, or a combination thereof.

In some aspects, provided herein, are kits for preparing induced hematopoietic stem cells comprising the following components: (a) a modified mRNA sequence encoding HOXB4; a modified mRNA sequence encoding PBX1; a modified mRNA sequence encoding LMO2; a modified mRNA sequence encoding ZFP467; and a modified mRNA sequence encoding ZFP521; wherein each cytosine of each of the modified mRNA sequences is a modified cytosine, each uracil of each of the modified mRNA sequences is a modified uracil, or a combination thereof; and (b) packaging and instructions therefor.

In some embodiments of these kits and all such kits described herein, the kit further comprises one or more of: a modified mRNA sequence encoding KLF12; a modified mRNA sequence encoding HLF; and a modified mRNA sequence encoding EGR; wherein each cytosine of each of the modified mRNA sequences is a modified cytosine, each uracil of each of the modified mRNA sequences is a modified uracil, or a combination thereof.

In some aspects, provided herein, are kits for preparing induced hematopoietic stem cells comprising the following components: (a) a modified mRNA sequence encoding MEIS1; a modified mRNA sequence encoding RBPMS; a modified mRNA sequence encoding ZFP37; a modified mRNA sequence encoding RUNX1T1; and a modified mRNA sequence encoding LMO2; wherein each cytosine of each of the modified mRNA sequences is a modified cytosine, each uracil of each of the modified mRNA sequences is a modified uracil, or a combination thereof; and (b) packaging and instructions therefor.

In some embodiments of these kits and all such kits described herein, the kit further comprises one or more of: a modified mRNA sequence encoding KLF12; and a modified mRNA sequence encoding HLF; wherein each cytosine of each of the modified mRNA sequences is a modified cytosine, each uracil of each of the modified mRNA sequences is a modified uracil, or a combination thereof.

In some aspects, provided herein, are kits for preparing induced hematopoietic stem cells comprising the following components: (a) a modified mRNA sequence encoding ZFP37; a modified mRNA sequence encoding HOXB4; a modified mRNA sequence encoding LMO2; and a modified mRNA sequence encoding HLF; wherein each cytosine of each of the modified mRNA sequences is a modified cytosine, each uracil of each of the modified mRNA sequences is a modified uracil, or a combination thereof; and (b) packaging and instructions therefor.

In some embodiments of these kits and all such kits described herein, the kit further comprises one or more of: a modified mRNA encoding MYCN; a modified mRNA encoding ZFP467; a modified mRNA encoding NKX2-3; a modified mRNA encoding PBX1; and a modified mRNA encoding KLF4; wherein each cytosine of each of the modified mRNA sequences is a modified cytosine, each uracil of each of the modified mRNA sequences is a modified uracil, or a combination thereof.

In some embodiments of these kits and all such kits described herein, the modified cytosine is 5-methylcytosine and the modified uracil is pseudouridine.

In some embodiments of these kits and all such kits described herein, one or more of the synthetic, modified mRNAs can further comprise one or more of a poly(A) tail, a Kozak sequence, a 3′ untranslated region, a 5′ untranslated regions, and a 5′ cap, such as 5′ cap analog, such as e.g., a 5′ diguanosine cap, tetraphosphate cap analogs having a methylene-bis(phosphonate) moiety, cap analogs having a sulfur substitution for a non-bridging oxygen, N7-benzylated dinucleoside tetraphosphate analogs, or anti-reverse cap analogs. The kits can also comprise a 5′ cap analog. The kit can also comprise a phosphatase enzyme (e.g., Calf intestinal phosphatase) to remove the 5′ triphosphate during the RNA modification procedure. Optionally, the kit can comprise one or more control synthetic mRNAs, such as a synthetic, modified RNA encoding green fluorescent protein (GFP) or other marker molecule.

In other embodiments, the kit can further comprise materials for further reducing the innate immune response of a cell. For example, the kit can further comprise a soluble interferon receptor, such as B18R. In some embodiments, the kit can comprise a plurality of different synthetic, modified RNA molecules.

The kits described herein can also comprise, in some aspects, one or more linear DNA templates for the generation of synthetic mRNAs encoding the HSC inducing factors described herein.

The kits described herein, in some embodiments, can further provide the synthetic mRNAs or the one or more expression vectors encoding HSC inducing factors in an admixture or as separate aliquots.

In some embodiments, the kits can further comprise an agent to enhance efficiency of reprogramming. In some embodiments, the kits can further comprise one or more antibodies or primer reagents to detect a cell-type specific marker to identify cells induced to the hematopoietic stem cell state.

In some embodiments, the kits can further comprise a buffer. In some such embodiments, the buffer is RNase-free TE buffer at pH 7.0. In some embodiments, the kit further comprises a container with cell culture medium.

All kits described herein can further comprise a buffer, a cell culture medium, a transduction or transfection medium and/or a media supplement. In preferred embodiments, the buffers, cell culture mediums, transfection mediums, and/or media supplements are DNAse and RNase-free. In some embodiments, the synthetic, modified RNAs provided in the kits can be in a non-solution form of specific quantity or mass, e.g., 20 μg, such as a lyophilized powder form, such that the end-user adds a suitable amount of buffer or medium to bring the components to a desired concentration, e.g., 100 ng/μl.

All kits described herein can further comprise devices to facilitate single-administration or repeated or frequent infusions of the cells generated using the kits components described herein, such as a non-implantable delivery device, e.g., needle, syringe, pen device, or an implantatable delivery device, e.g., a pump, semi-permanent stent (e.g., intravenous, intraperitoneal, intracisternal or intracapsular), or reservoir. In some such embodiments, the delivery device can include a mechanism to dispense a unit dose of a pharmaceutical composition comprising the iHSC clone. In some embodiments, the device releases the composition continuously, e.g., by diffusion. In some embodiments, the device can include a sensor that monitors a parameter within a subject. For example, the device can include pump, e.g., and, optionally, associated electronics.

The induced hematopoietic stem cells in some aspects of all the embodiments of the invention, while similar in functional characteristics, differ significantly in their gene expression or methylation pattern from the naturally occurring endogenous hematopoietic stem cells. For example, compared to the endogenous HSC gene expression pattern, exemplary genes of which are shown in Tables 2 and 3, the induced hematopoietic stem cells differ by showing about 1-5%, 5-10%, 5-15%, or 5-20% increased expression of about 1-5%, 2-5%, 3-5%, up to 50%, up to 40%, up to 30%, up to 25%, up to 20%, up to 15%, or up to 10% of the genes in endogenous HSCs, for example, those set forth in Tables 2 and 3. Specifically, the expression in the iHSCs of genes the expression of which is reduced or insignificant in the naturally occurring HSCs (see, selected examples in Table 2), is increased or the expression of the genes the expression of which is significant in the naturally occurring HSCs (see, selected examples of highly expressed genes in isolated HSCs in Table 3) is decreased in iHSCs.

In some aspects of all the embodiments of the invention, while similar in functional characteristics, the induced pluripotent stem cells differ significantly in their methylation pattern from the naturally occurring or endogenous HSCs. For example, compared to the endogenous methylation pattern of genes as exemplified in Table 4, the iHSCs differ by showing about 1-5%, in some aspects 1-10%, in some aspects 5-10% difference in the methylation of at about 1-5%, 1-10%, 5-10%, up to 50%, up to 40%, up to 30%, up to 25%, up to 20%, up to 15%, or up to 10% of the methylation sites of naturally occurring HSCs, which are exemplified in Table 4. The difference may be increased or decreased methylation compared to endogenous HSCs. In some aspects, some methylation sites are methylated and some unmethylated in iHSCs compared to the endogenous HSCs methylation sites as exemplified in Table 4.

Table 4 includes 35 exemplary profiles from each chromosome (1-19, x and y) as profiled in naturally occurring or endogenous HSCs. The screening was done by randomizing the most and least methylated sites (i.e. the top/bottom 20%) where 100 were taken from each group (except the Y chromosome which had a very small number of sites and only 35 random sites were selected). Of the mid (20-80%) percentiles, 3000 methylation sites were randomly selected. From this pool of 3000 sites, 35 methylation sites were randomly selected. These examples were selected to represent the methylation status of the entire chromosome but enrich for those mid-range sites of methylation which, without wishing to be bound by theory, may be more characteristic of the naturally occurring HSC.

### HSC Expression Analysis

Genome-wide gene expression analysis was performed on purified LSKCD34-Flk2-using the Affymetrix GeneChip Mouse Genome 430 2.0 Array platform. RNA was isolated using TRIzol (Life Technologies) and purified RNA was amplified, labeled, hybridized, and scanned according to Affymetrix's. Raw data was normalized using gcRMA together with 383 other hematopoietic cell types. These data were log transformed and average of the four biological replicates of are presented as expression levels.

### DNA Methylation Analysis of HSCs

RRBS libraries for DNA methylation analysis were prepared from 30 ng input DNA per biological replicate of LSKCD34-FLk2-HSCs following a published protocol (Gu et al Nat. Protoc, 6 (2011), pp. 468-481) and sequenced by the Broad Institute's Genome Sequencing Platform on Illumina Genome Analyzer II or HiSeq 2000 machines. Bioinformatic data processing and quality control were performed as described in Bock et al (Cell, 144 (2011), pp. 439-452). The raw sequencing reads were aligned using Maq's bisulfite alignment mode and DNA methylation calling was performed using custom software (Gu et al, Nat Methods 7(2010) 133-136). DNA methylation levels were calculated for 1-kilobase tiling regions throughout the genome as coverage-weighted means of the DNA methylation levels of individual CpGs. Only regions with at least two CpGs with at least 5 independent DNA methylation measurements per CpG were retained, giving rise to a list of genomic regions with high-confidence DNA methylation measurements. In the initial filtering step, all 1-kb tiles of DNA methylation were excluded for which the two biological replicates were not sufficiently consistent with each other. Any measurement was excluded if the absolute divergence between biological replicates exceeded 0.2 and if the relative divergence between biological replicates exceeded 0.05. These absolute thresholds were selected based on our previous experience with RRBS data analysis, and the relative thresholds were calculated such that the absolute and relative thresholds became equivalent for values close to the center of the spectrum, i.e. around 0.5. Identification of significant differentially methylated regions were based on the average DNA methylation difference between the biological replicates of two cell types, requiring a minimum absolute difference of 0.1 for 1-kb tiles, and a more stringent threshold of 0.2 for single CpGs. The relative difference thresholds were calculated from the absolute difference thresholds as described above. The combined use of relative and absolute difference thresholds resulted in robust identification of relevant differences across the spectrum of genes and genomic regions with high, medium and low DNA methylation.

TABLE 2
Examples of transcripts showing reduced/
insignificant expression in endogenous HSCs

| | Expression | |
|---|---|---|
| | (Average of 4 | |
| | datasets of | |
| Probeset | purified HSCs) | Gene Symbol |
| 1425771_at | 4.65 | Akr1d1 |
| 1425772_at | 4.65 | Col4a4 |
| 1425773_s_at | 4.65 | Nmnat1 |
| 1425774_at | 4.65 | Srrm4 |
| 1425775_at | 4.65 | Zfp820 |
| 1425776_a_at | 4.65 | C87436 |
| 1425777_at | 4.65 | Cacnb1 |
| 1425778_at | 4.65 | Ido2 |
| 1425779_a_at | 4.65 | Tbx1 |
| 1425780_a_at | 4.65 | Tmem167 |
| 1425781_a_at | 4.65 | Plcb1 |
| 1425782_at | 4.65 | Plcb1 |
| 1425783_at | 4.65 | Tc2n |
| 1425784_a_at | 4.65 | Olfm1 |
| 1425785_a_at | 4.65 | Txk |
| 1425786_a_at | 4.65 | Hsf4 |
| 1425787_a_at | 4.65 | Sytl3 |
| 1425788_a_at | 4.65 | Echdc2 |
| 1425789_s_at | 4.65 | Anxa8 |
| 1425790_a_at | 4.65 | Grik2 |
| 1425791_at | 4.65 | Pon2 |
| 1425792_a_at | 4.65 | Rorc |
| 1425793_a_at | 4.65 | Rorc |
| 1425794_at | 4.65 | Pola2 |
| 1425795_a_at | 4.65 | Map3k7 |
| 1425796_a_at | 4.65 | Fgfr3 |
| 1425797_a_at | 4.65 | Syk |
| 1425808_a_at | 4.65 | Myocd |
| 1425798_a_at | 4.65 | Recql |
| 1425800_at | 4.65 | Rad9b |
| 1425801_x_at | 4.65 | Cotl1 |
| 1425802_a_at | 4.65 | Fcrla |
| 1425803_a_at | 4.65 | Mbd2 |
| 1425804_at | 4.65 | Hmx2 |
| 1425806_a_at | 4.65 | Med21 |
| 1425807_at | 4.65 | BC021891 |
| 1425809_at | 4.65 | Fabp4 |
| 1425810_a_at | 4.65 | Csrp1 |
| 1425811_a_at | 4.65 | Csrp1 |
| 1425812_a_at | 4.65 | Cacna1b |
| 1425813_at | 4.65 | Pign |
| 1425814_a_at | 4.65 | Calcrl |
| 1425815_a_at | 4.65 | Hmmr |
| 1425816_at | 4.65 | Zfp287 |
| 1425817_a_at | 4.65 | Slc8a1 |
| 1425818_at | 4.65 | 4930520O04Rik |
| 1425819_at | 4.65 | Zbtb7c |
| 1425820_x_at | 4.65 | Gpatch4 |
| 1425821_at | 4.65 | Clcn7 |
| 1425822_a_at | 4.65 | Dtx1 |
| 1426032_at | 4.65 | Nfatc2 |
| 1425823_at | 4.65 | Cfhr2 |
| 1425825_at | 4.65 | Eml6 |
| 1425826_a_at | 4.65 | Sorbs1 |
| 1425827_at | 4.65 | Nkx2-3 |
| 1425828_at | 4.65 | Nkx6-1 |
| 1425829_a_at | 4.65 | Steap4 |
| 1425830_a_at | 4.65 | Cinp /// LOC640972 |
| 1425831_at | 4.65 | Zfp101 |
| 1425832_a_at | 4.65 | Cxcr6 |
| 1425833_a_at | 4.65 | Hpca |
| 1425834_a_at | 4.65 | Gpam |
| 1425835_a_at | 4.65 | Bbx |
| 1425836_a_at | 4.65 | Limk1 |
| 1425837_a_at | 4.65 | Ccrn4l |
| 1425838_at | 4.65 | Atp9a |
| 1425839_at | 4.65 | Fkbp11 |
| 1425840_a_at | 4.65 | Sema3f |
| 1425842_at | 4.65 | Edil3 |
| 1425843_at | 4.65 | Mrpl33 |
| 1425845_a_at | 4.65 | Shoc2 |
| 1425846_a_at | 4.65 | Caln1 |
| 1425848_a_at | 4.65 | Dusp26 |
| 1425849_at | 4.65 | Chrnb4 |
| 1425850_a_at | 4.65 | Nek6 |
| 1425851_a_at | 4.65 | Amigo1 |
| 1425852_at | 4.65 | Catsperg1 |
| 1425855_a_at | 4.65 | Crk |
| 1425857_at | 4.65 | Fbxw9 |
| 1425858_at | 4.65 | Ube2m |
| 1425859_a_at | 4.65 | Psmd4 |
| 1425861_x_at | 4.65 | Cacna2d1 |
| 1425863_a_at | 4.65 | Ptpro |
| 1425864_a_at | 4.65 | Sorcs1 |
| 1425865_a_at | 4.65 | Lig3 |
| 1425866_a_at | 4.65 | Plekha4 |
| 1425867_at | 4.65 | Plekha4 |
| 1425868_at | 4.65 | Hist2h2bb |
| 1425869_a_at | 4.65 | Psen2 |
| 1425870_a_at | 4.65 | Kcnip2 |
| 1425871_a_at | 4.65 | Igk-V28 |
| 1425874_at | 4.65 | Hoxc13 |
| 1425875_a_at | 4.65 | Lepr |
| 1425876_a_at | 4.65 | Glce |
| 1425877_at | 4.65 | Hyal3 |
| 1425878_at | 4.65 | Cabp4 |
| 1425879_at | 4.65 | Zfp352 |
| 1425880_x_at | 4.65 | Zfp352 |
| 1425881_at | 4.65 | Psg28 |
| 1425882_at | 4.65 | Gdf2 |
| 1425883_at | 4.65 | Smg6 |
| 1425884_at | 4.65 | Rpf2 |
| 1425885_a_at | 4.65 | Kcnab2 |
| 1425888_at | 4.65 | Klra17 |
| 1425889_at | 4.65 | Wnt9a |
| 1425890_at | 4.65 | Ly6i |
| 1425891_a_at | 4.65 | Grtp1 |
| 1425893_a_at | 4.65 | Fhit |
| 1425895_a_at | 4.65 | Id1 |
| 1425897_at | 4.65 | — |
| 1425898_x_at | 4.65 | Olfm3 |
| 1425899_a_at | 4.65 | Itsn1 |
| 1425901_at | 4.65 | Nfatc2 |
| 1425903_at | 4.65 | Sema6a |
| 1425904_at | 4.65 | Satb2 |
| 1425905_at | 4.65 | — |
| 1425906_a_at | 4.65 | Sema3e |
| 1425907_s_at | 4.65 | Amot |
| 1425908_at | 4.65 | Gnb1 |
| 1425910_at | 4.65 | Dnajc2 |
| 1425911_a_at | 4.65 | Fgfr1 |
| 1425912_at | 4.65 | Cep164 |
| 1425913_a_at | 4.65 | Spats2l |
| 1425914_a_at | 4.65 | Armcx1 |
| 1425915_at | 4.65 | Slc26a8 |
| 1425916_at | 4.65 | Capn8 |
| 1425917_at | 4.65 | H28 |
| 1425918_at | 4.65 | — |
| 1425919_at | 4.65 | Ndufa12 |
| 1425920_at | 4.65 | Cuedc1 |
| 1425921_a_at | 4.65 | 1810055G02Rik |
| 1425922_a_at | 4.65 | Mycn |
| 1425923_at | 4.65 | Mycn |
| 1425925_at | 4.65 | Fcamr |
| 1425926_a_at | 4.65 | Otx2 |
| 1425927_a_at | 4.65 | Atf5 |
| 1425928_at | 4.65 | Xkr6 |
| 1425929_a_at | 4.65 | Rnf14 |
| 1425931_a_at | 4.65 | Arntl2 |
| 1425932_a_at | 4.65 | Celf1 |
| 1425934_a_at | 4.65 | B4galt4 |
| 1425935_at | 4.65 | Hspb11 |
| 1425936_a_at | 4.65 | Ankmy2 |
| 1425937_a_at | 4.65 | Hexim1 |
| 1425939_at | 4.65 | Rad50 |
| 1425940_a_at | 4.65 | Ssbp3 |
| 1425941_a_at | 4.65 | Fanci |
| 1425942_a_at | 4.65 | Gpm6b |
| 1425943_at | 4.65 | Nmur2 |
| 1425944_a_at | 4.65 | Rad51l3 |
| 1425945_at | 4.65 | Zfp626 |
| 1425946_at | 4.65 | Gstm7 |
| 1425947_at | 4.65 | Ifng |
| 1425949_at | 4.65 | Slc25a30 |
| 1425950_at | 4.65 | Slc17a9 |
| 1425951_a_at | 4.65 | Clec4n |
| 1425952_a_at | 4.65 | Gcg |
| 1425953_at | 4.65 | — |
| 1425954_a_at | 4.65 | Apex2 |
| 1425955_at | 4.65 | Cav2 |
| 1425958_at | 4.65 | Il1f9 |
| 1425959_x_at | 4.65 | Klra16 |
| 1425960_s_at | 4.65 | Pax6 |
| 1425962_at | 4.65 | Klrb1f |
| 1425963_at | 4.65 | Cabp7 |
| 1425964_x_at | 4.65 | Hspb1 |
| 1425965_at | 4.65 | Ubc |
| 1425966_x_at | 4.65 | Ubc |
| 1425967_a_at | 4.65 | Mcpt4 |
| 1425968_s_at | 4.65 | Speg |
| 1425969_a_at | 4.65 | Htt |
| 1425970_a_at | 4.65 | Ros1 |
| 1425971_at | 4.65 | Naip3 |
| 1425972_a_at | 4.65 | Zfx |
| 1425973_at | 4.65 | Lyst |
| 1425975_a_at | 4.65 | Mapk8ip3 |
| 1426023_a_at | 4.65 | Rabep1 |
| 1426024_a_at | 4.65 | Dbn1 |
| 1426025_s_at | 4.65 | Laptm5 |
| 1425976_x_at | 4.65 | Zfp353 |
| 1425977_a_at | 4.65 | Slk |
| 1425979_a_at | 4.65 | Fbf1 |
| 1425980_at | 4.65 | Wdr54 |
| 1425981_a_at | 4.65 | Rbl2 |
| 1425983_x_at | 4.65 | Hipk2 |
| 1425985_s_at | 4.65 | Masp1 |
| 1425986_a_at | 4.65 | Dcun1d1 |
| 1425987_a_at | 4.65 | Kcnma1 |
| 1425988_a_at | 4.65 | Hipk1 |
| 1425989_a_at | 4.65 | Eya3 |
| 1425990_a_at | 4.65 | Nfatc2 |
| 1425991_a_at | 4.65 | Kank2 |
| 1425992_at | 4.65 | Slc6a5 |
| 1425994_a_at | 4.65 | Asah2 |
| 1425995_s_at | 4.65 | Wt1 |
| 1425996_a_at | 4.65 | Hltf |
| 1425997_a_at | 4.65 | Pign |
| 1425998_at | 4.65 | Sytl4 |
| 1426001_at | 4.65 | Eomes |
| 1426004_a_at | 4.65 | Tgm2 |
| 1426005_at | 4.65 | Dmp1 |
| 1426006_at | 4.65 | Kcnq2 |
| 1426008_a_at | 4.65 | Slc7a2 |
| 1426009_a_at | 4.65 | Pip5k1a |
| 1426010_a_at | 4.65 | Epb4.1l3 |
| 1426011_a_at | 4.65 | Ggnbp2 |
| 1426012_a_at | 4.65 | 2610301G19Rik |
| 1426013_s_at | 4.65 | Plekha4 |
| 1426014_a_at | 4.65 | Cdhr5 |
| 1426017_a_at | 4.65 | 0610011L14Rik |
| 1426018_a_at | 4.65 | Sox6 |
| 1426019_at | 4.65 | Plaa |
| 1426021_a_at | 4.65 | Cdc7 |
| 1426022_a_at | 4.65 | Vill |
| 1426026_at | 4.65 | Prpf6 |
| 1426027_a_at | 4.65 | Arhgap10 |
| 1426028_a_at | 4.65 | Cit |

TABLE 3
Examples of transcripts showing expression/
significant expression in endogenous HSCs

| | Expression (Average of 4 datasets | |
|---|---|---|
| Probeset | of purified HSCs) | Gene Symbol |
| 1424256_at | 100879.78 | Rdh12 |
| 1424539_at | 79795.71 | Ubl4 |
| 1420954_a_at | 76447.45 | Add1 |
| 1421742_at | 75395.99 | — |
| 1424295_at | 72899.90 | Dppa3 |
| 1423567_a_at | 72869.27 | Psma7 |
| 1423106_at | 70905.48 | Ube2b |
| 1424391_at | 69677.87 | Nrd1 |
| 1424069_at | 69512.25 | Napg |
| 1424721_at | 67140.32 | Mfap3 |
| 1422960_at | 65644.79 | Srd5a2 |
| 1421948_a_at | 64085.44 | Ccdc123 |
| 1423089_at | 62549.13 | Tmod3 |
| 1424335_at | 62005.99 | Ppcdc |
| 1423792_a_at | 60183.19 | Cmtm6 |
| 1422398_at | 58720.84 | Hist1h1e |
| 1421896_at | 58579.47 | Elk1 |
| 1423355_at | 57569.64 | Snap29 |
| 1420529_at | 57554.85 | Dpf1 |
| 1423240_at | 57379.26 | Src |
| 1421410_a_at | 56489.03 | Pstpip2 |
| 1421584_at | 54335.88 | Opn4 |
| 1420202_at | 54182.06 | — |
| 1422376_at | 54014.33 | Vmn1r50 |
| 1423848_at | 53959.70 | Mphosph6 |
| 1422416_s_at | 53943.95 | Vpreb1///Vpreb2 |
| 1423907_a_at | 53750.78 | Ndufs8 |
| 1419015_at | 52526.85 | Wisp2 |
| 1422702_at | 52048.42 | Azin1 |
| 1423817_s_at | 51920.82 | Use1 |
| 1422664_at | 51789.77 | Rab10 |
| 1421988_at | 51730.79 | Papss2 |
| 1420092_at | 51443.43 | Morc3 |
| 1419919_at | 50903.42 | — |
| 1423493_a_at | 50864.75 | Nfix |
| 1420517_at | 49770.55 | Chmp4c |
| 1422490_at | 49492.67 | Bnip2 |
| 1423805_at | 49225.38 | Dab2 |
| 1421893_a_at | 49082.98 | Tpp2 |
| 1422607_at | 48373.32 | Etv1 |
| 1422808_s_at | 48260.89 | Dock2 |
| 1423728_at | 47793.86 | Eif3l |
| 1422634_a_at | 47057.45 | Vsig2 |
| 1423415_at | 46829.97 | Gpr83 |
| 1423774_a_at | 46597.55 | Prc1 |
| 1421205_at | 46410.24 | Atm |
| 1422725_at | 46373.82 | Mak |
| 1422876_at | 46000.03 | Capn9 |
| 1420030_at | 45773.96 | Slu7 |
| 1423082_at | 45717.01 | Derl1 |
| 1424369_at | 45609.09 | Psmf1 |
| 1424432_at | 45430.90 | Ubtd1 |
| 1421578_at | 45382.12 | Ccl4 |
| 1422729_at | 45325.62 | Pcdhb10 |
| 1424004_x_at | 45166.17 | 4930444A02Rik |
| 1419676_at | 45159.39 | Mx2 |
| 1422946_a_at | 45067.84 | Dnmt1 |
| 1420200_at | 44965.21 | — |
| 1421868_a_at | 44891.20 | Pnlip |
| 1420217_x_at | 44808.32 | — |
| 1419864_x_at | 44771.30 | Tnpo1 |
| 1432675_at | 44721.78 | Mdn1 |
| 1423206_s_at | 44538.34 | 2310003F16Rik/// |
| | | Serf2 |
| 1423402_at | 44427.28 | Creb1 |
| 1420539_a_at | 43572.89 | Chrdl2 |
| 1423072_at | 43569.21 | 6720475J19Rik |
| 1423348_at | 43334.95 | Fzd8 |
| 1422152_at | 43301.54 | Hmx1 |
| 1420955_at | 42958.08 | Vsnl1 |
| 1422534_at | 42719.81 | Cyp51 |
| 1421514_a_at | 42690.03 | Scml2 |
| 1420573_at | 42424.32 | Hoxd1 |
| 1422139_at | 42321.56 | Plau |
| 1423193_at | 42255.15 | Pspc1 |
| 1422949_at | 41969.65 | Nos1 |
| 1422585_at | 41579.30 | Odf1 |
| 1421685_at | 41540.59 | Clec4b1 |
| 1421144_at | 41368.55 | Rpgrip1 |
| 1422038_a_at | 41364.86 | Tnfrsf22 |
| 1425165_at | 41318.16 | Gzmn |
| 1425101_a_at | 41263.26 | Fkbp6 |
| 1421858_at | 40782.82 | Adam17 |
| 1424361_at | 40305.18 | Tti2 |
| 1432026_a_at | 39842.37 | Herc6 |
| 1421877_at | 39450.73 | Mapk9 |
| 1424168_a_at | 39344.00 | Capzb |
| 1423746_at | 39125.86 | Txndc5 |
| 1421784_a_at | 39087.91 | Efna4 |
| 1422216_at | 38969.12 | Mid2 |
| 1437495_at | 38891.23 | Mbtps2///Yy2 |
| 1422193_at | 38621.58 | Gucy2e |
| 1424209_at | 38397.04 | Rars2 |
| 1421734_at | 38265.53 | Cxcr2 |
| 1422764_at | 38046.45 | Mapre1 |
| 1422461_at | 37752.66 | Atad3a |
| 1422319_at | 37656.70 | — |
| 1421828_at | 37384.32 | Kpna3 |
| 1422947_at | 37379.83 | Hist1h4a |
| 1417187_at | 37147.52 | Ube2k |
| 1420237_at | 37138.69 | — |
| 1421111_at | 37129.17 | Rybp |
| 1421762_at | 36844.59 | Kcnj5 |
| 1425001_at | 36814.72 | Rnf146 |
| 1422763_at | 36738.09 | Gipc1 |
| 1421198_at | 36633.80 | Itgav |
| 1423022_at | 36619.85 | Adra2a |
| 1425460_at | 36318.33 | Mtmr2 |
| 1423718_at | 35541.24 | Ak3 |
| 1424746_at | 35456.02 | Kif1c |
| 1422791_at | 35371.28 | Pafah1b2 |
| 1443492_at | 35208.55 | — |
| 1422154_at | 35197.92 | Gpr27 |
| 1423232_at | 35156.06 | Etv4 |
| 1434987_at | 34983.28 | Aldh2 |
| 1421928_at | 34894.19 | Epha4 |
| 1421276_a_at | 34783.78 | Dst |
| 1418807_at | 34723.24 | 3110070M22Rik |
| 1421357_at | 34509.96 | Gtf2a1 |
| 1420450_at | 33787.26 | Mmp10 |
| 1425562_s_at | 33760.26 | Trnt1 |
| 1422137_at | 33732.68 | Duoxa2 |
| 1420882_a_at | 33268.28 | Acd |
| 1420792_at | 32727.55 | 4930433N12Rik |
| 1428618_at | 32608.49 | Hcfc2 |
| 1423324_at | 32498.13 | Pnn |
| 1421066_at | 32380.36 | Jak2 |
| 1421767_at | 32357.95 | Adk |
| 1423465_at | 32223.80 | Frrs1 |
| 1420412_at | 32006.60 | Tnfsf10 |
| 1422403_at | 31627.13 | Gm12597 |
| 1420644_a_at | 31555.81 | Sec61a2 |
| 1424157_at | 31355.35 | Ehd2 |
| 1425678_a_at | 31211.98 | Snrk |
| 1419171_at | 30993.36 | Fam174a |
| 1424059_at | 30975.22 | Suv420h2 |
| 1423390_at | 30941.65 | Siah1a |
| 1430244_at | 30636.46 | 4921509J17Rik |
| 1424356_a_at | 30596.60 | Metrnl |
| 1422035_at | 30526.30 | Serpinb9c |
| 1424763_at | 30455.13 | Rsph9 |
| 1420242_at | 30259.70 | — |
| 1423292_a_at | 30255.63 | Prx |
| 1425719_a_at | 30011.99 | Nmi |
| 1422891_at | 29811.27 | H2-Ea-ps |
| 1433073_at | 29755.02 | 4933425E08Rik |
| 1424874_a_at | 29586.89 | Ptbp1 |
| 1421795_s_at | 29485.47 | Klrc2///Klrc3 |
| 1424781_at | 29441.10 | Reep3 |
| 1420106_at | 29316.87 | Siah1a |
| 1423735_a_at | 29115.24 | Wdr36 |
| 1421132_at | 28979.38 | Pvrl3 |
| 1423440_at | 28884.32 | Fam33a |
| 1424619_at | 28807.35 | Sf3b4 |
| 1420359_at | 28678.72 | Sva |
| 1422121_at | 28666.64 | Oprd1 |
| 1424773_at | 28663.97 | Fam125a |
| 1422217_a_at | 28522.13 | Cyp1a1 |
| 1419908_at | 28487.43 | Fcrla |
| 1416576_at | 27695.03 | Socs3 |
| 1422574_at | 27639.56 | Mxd4 |
| 1433622 at | 27471.80 | Gemin4///Glod4/// |
| | | Gm6330 |
| 1438263_at | 27434.33 | 9430020K01Rik |
| 1425220_x_at | 27306.78 | LOC100038937 |
| 1422454_at | 27268.17 | Krt13 |
| 1422240_s_at | 26926.68 | Sprr2h |
| 1433942_at | 26894.49 | Myo6 |
| 1437613_s_at | 26870.76 | Ptpdc1 |
| 1418969_at | 26582.64 | Skp2 |
| 1421818_at | 26510.49 | Bcl6 |
| 1422017_s_at | 26492.47 | 4833439L19Rik |
| 1422088_at | 26321.36 | Mycl1 |
| 1424911_a_at | 26252.42 | Lyzl4 |
| 1415812_at | 26042.95 | Gsn |
| 1422592_at | 25974.74 | Ctnnd2 |
| 1421422_at | 25602.36 | 5033411D12Rik |
| 1422511_a_at | 25483.54 | Ogfr |
| 1432823_at | 25438.68 | Sypl2 |
| 1421211_a_at | 25380.22 | Ciita |
| 1416578_at | 25267.25 | Gm9840///Rbx1 |
| 1425535_at | 25144.30 | Repin1 |
| 1420466_at | 25061.79 | Mucl1 |
| 1437720_at | 24921.64 | Eif2d |
| 1422435_at | 24867.70 | 2210010C04Rik |
| 1420648_at | 24760.09 | Trim12a |
| 1421382_at | 24658.48 | Prlr |
| 1416404_s_at | 24652.70 | Rps16 |
| 1424118_a_at | 24646.84 | Spc25 |
| 1425180_at | 24391.49 | Sgip1 |
| 1422621_at | 24276.19 | Ranbp2 |
| 1421265_a_at | 24108.68 | Rbm38 |
| 1423590_at | 23955.37 | Napsa |
| 1431842_at | 23948.99 | 4930422C21Rik |
| 1428567_at | 23851.44 | Hspbap1 |
| 1424928_at | 23715.06 | 2210018M11Rik |
| 1421894_a_at | 23697.49 | Tpp2 |
| 1420489_at | 23628.96 | Mrps14 |
| 1425406_at | 23574.24 | Clec4a2 |
| 1419907_s_at | 23407.93 | Fcrla |
| 1421139_a_at | 23222.94 | Zfp386 |
| 1420219_at | 23098.02 | Dnajc21 |
| 1420714_at | 23021.11 | Lbx2 |
| 1419571_at | 23014.90 | Slc28a3 |
| 1424501_at | 22942.41 | Utp6 |
| 1423777_at | 22813.47 | Usp20 |
| 1424712_at | 22776.38 | Ahctf1 |
| 1421693_a_at | 22651.12 | Gpr98 |
| 1437991_x_at | 22601.85 | Rusc1 |
| 1418666_at | 22593.56 | Ptx3 |
| 1420348_at | 22525.87 | Lhx5 |
| 1422735_at | 22457.19 | Foxq1 |
| 1424455_at | 22297.49 | Gprasp1 |
| 1420446_at | 22176.11 | Odf3 |
| 1420207_at | 22023.74 | — |
| 1421363_at | 21974.00 | Cyp2c39 |

TABLE 4
Exemplary methylation sites in isolated/endogenous HSCs

| Chr. | Chr. Start | Chr. End | Name | Gene Name | EnsemblId | HSC |
|---|---|---|---|---|---|---|
| chr1 | 38475000 | 38476000 | 35378 | Rev1 | ENSMUSG00000026082 | 0.971 |
| chr1 | 174135000 | 174136000 | 168890 | Dcaf8 | ENSMUSG00000026554 | 0.663 |
| chr1 | 187516000 | 187517000 | 181864 | Slc30a10 | ENSMUSG00000026614 | 0.540 |
| chr1 | 190087000 | 190088000 | 184435 | Ush2a | ENSMUSG00000026609 | 0.974 |
| chr1 | 38011000 | 38012000 | 34914 | Lyg2 | ENSMUSG00000061584 | 0.612 |
| chr1 | 36290000 | 36291000 | 33193 | Hs6st1 | ENSMUSG00000045216 | 0.522 |
| chr1 | 91946000 | 91947000 | 86834 | Asb18 | ENSMUSG00000067081 | 0.576 |
| chr1 | 91825000 | 91826000 | 86713 | Agap1 | ENSMUSG00000055013 | 0.365 |
| chr1 | 12966000 | 12967000 | 9967 | Sulf1 | ENSMUSG00000016918 | 0.596 |
| chr1 | 191714000 | 191715000 | 186062 | Ptpn14 | ENSMUSG00000026604 | 0.994 |
| chr1 | 94962000 | 94963000 | 89850 | Aqp12 | ENSMUSG00000045091 | 0.604 |
| chr1 | 36355000 | 36356000 | 33258 | Neurl3 | ENSMUSG00000047180 | 0.539 |
| chr1 | 34593000 | 34594000 | 31496 | Cfc1 | ENSMUSG00000026124 | 0.211 |
| chr1 | 185803000 | 185804000 | 180151 | Tlr5 | ENSMUSG00000079164 | 0.213 |
| chr1 | 74195000 | 74196000 | 71098 | Rufy4 | ENSMUSG00000061815 | 0.610 |
| chr1 | 90736000 | 90737000 | 85624 | Arl4c | ENSMUSG00000049866 | 0.653 |
| chr1 | 191658000 | 191659000 | 186006 | Ptpn14 | ENSMUSG00000026604 | 0.974 |
| chr1 | 191661000 | 191662000 | 186009 | Ptpn14 | ENSMUSG00000026604 | 0.968 |
| chr1 | 38579000 | 38580000 | 35482 | Rev1 | ENSMUSG00000026082 | 0.969 |
| chr1 | 127809000 | 127810000 | 122697 | Lypd1 | ENSMUSG00000026344 | 0.213 |
| chr1 | 25234000 | 25235000 | 22137 | Lmbrd1 | ENSMUSG00000073725 | 0.550 |
| chr1 | 191952000 | 191953000 | 186300 | Smyd2 | ENSMUSG00000026603 | 0.658 |
| chr1 | 91954000 | 91955000 | 86842 | Asb18 | ENSMUSG00000067081 | 0.980 |
| chr1 | 188658000 | 188659000 | 183006 | Rrp15 | ENSMUSG00000001305 | 0.000 |
| chr1 | 34308000 | 34309000 | 31211 | Dst | ENSMUSG00000026131 | 0.365 |
| chr1 | 137815000 | 137816000 | 132703 | Pkp1 | ENSMUSG00000026413 | 0.035 |
| chr1 | 191583000 | 191584000 | 185931 | Ptpn14 | ENSMUSG00000026604 | 0.979 |
| chr1 | 14812000 | 14813000 | 11813 | Msc | ENSMUSG00000025930 | 0.587 |
| chr1 | 94547000 | 94548000 | 89435 | Otos | ENSMUSG00000044055 | 0.795 |
| chr1 | 36327000 | 36328000 | 33230 | Uggt1 | ENSMUSG00000037470 | 0.150 |
| chr1 | 90701000 | 90702000 | 85589 | Ar14c | ENSMUSG00000049866 | 0.893 |
| chr1 | 40212000 | 40213000 | 37115 | Il1r2 | ENSMUSG00000026073 | 0.970 |
| chr1 | 140473000 | 140474000 | 135361 | Atp6v1g3 | ENSMUSG00000026394 | 0.599 |
| chr1 | 90565000 | 90566000 | 85453 | Glrp1 | ENSMUSG00000062310 | 0.564 |
| chr1 | 51516000 | 51517000 | 48419 | Sdpr | ENSMUSG00000045954 | 0.707 |
| chr2 | 163597000 | 163598000 | 351938 | Ada | ENSMUSG00000017697 | 0.588 |
| chr2 | 29297000 | 29298000 | 217736 | Med27 | ENSMUSG00000026799 | 0.969 |
| chr2 | 170120000 | 170121000 | 358461 | | ENSMUSG00000084013 | 0.640 |
| chr2 | 170332000 | 170333000 | 358673 | Cyp24a1 | ENSMUSG00000038567 | 0.553 |
| chr2 | 63809000 | 63810000 | 252199 | | ENSMUSG00000065837 | 0.612 |
| chr2 | 143610000 | 143611000 | 331951 | Pcsk2 | ENSMUSG00000027419 | 0.894 |
| chr2 | 163321000 | 163322000 | 351662 | R3hdml | ENSMUSG00000078949 | 0.795 |
| chr2 | 147874000 | 147875000 | 336215 | Foxa2 | ENSMUSG00000037025 | 0.030 |
| chr2 | 151719000 | 151720000 | 340060 | Rspo4 | ENSMUSG00000032852 | 0.482 |
| chr2 | 170107000 | 170108000 | 358448 | Zfp217 | ENSMUSG00000052056 | 0.650 |
| chr2 | 101484000 | 101485000 | 289874 | | ENSMUSG00000027165 | 0.969 |
| chr2 | 157964000 | 157965000 | 346305 | Rprd1b | ENSMUSG00000027651 | 0.974 |
| chr2 | 162773000 | 162774000 | 351114 | L3mbtl | ENSMUSG00000035576 | 0.573 |
| chr2 | 82981000 | 82982000 | 271371 | | ENSMUSG00000075248 | 0.640 |
| chr2 | 165999000 | 166000000 | 354340 | Sulf2 | ENSMUSG00000006800 | 0.795 |
| chr2 | 29061000 | 29062000 | 217500 | Setx | ENSMUSG00000043535 | 0.622 |
| chr2 | 173161000 | 173162000 | 361500 | Pmepa1 | ENSMUSG00000038400 | 0.036 |
| chr2 | 92582000 | 92583000 | 280972 | Chst1 | ENSMUSG00000027221 | 0.381 |
| chr2 | 160803000 | 160804000 | 349144 | Emilin3 | ENSMUSG00000050700 | 0.976 |
| chr2 | 57034000 | 57035000 | 245473 | Nr4a2 | ENSMUSG00000026826 | 0.002 |
| chr2 | 153116000 | 153117000 | 341457 | Pofut1 | ENSMUSG00000046020 | 0.510 |
| chr2 | 37898000 | 37899000 | 226337 | Crb2 | ENSMUSG00000035403 | 0.971 |
| chr2 | 78788000 | 78789000 | 267178 | Ube2e3 | ENSMUSG00000027011 | 0.640 |
| chr2 | 152737000 | 152738000 | 341078 | Mylk2 | ENSMUSG00000027470 | 0.465 |
| chr2 | 127978000 | 127979000 | 316319 | Bcl2l11 | ENSMUSG00000027381 | 0.532 |
| chr2 | 34060000 | 34061000 | 222499 | Fam125b | ENSMUSG00000038740 | 0.990 |
| chr2 | 38079000 | 38080000 | 226518 | Crb2 | ENSMUSG00000035403 | 0.621 |
| chr2 | 152831000 | 152832000 | 341172 | Ttll9 | ENSMUSG00000074673 | 0.971 |
| chr2 | 151272000 | 151273000 | 339613 | | ENSMUSG00000083391 | 0.645 |
| chr2 | 32730000 | 32731000 | 221169 | Stxbp1 | ENSMUSG00000026797 | 0.115 |
| chr2 | 35302000 | 35303000 | 223741 | Ggta1 | ENSMUSG00000035778 | 0.402 |
| chr2 | 173251000 | 173252000 | 361590 | Pmepa1 | ENSMUSG00000038400 | 0.643 |
| chr2 | 26338000 | 26339000 | 214777 | Sec16a | ENSMUSG00000026924 | 0.530 |
| chr2 | 131778000 | 131779000 | 320119 | Prnd | ENSMUSG00000027338 | 0.131 |
| chr2 | 26436000 | 26437000 | 214875 | Egfl7 | ENSMUSG00000026921 | 0.641 |
| chr3 | 102264000 | 102265000 | 469052 | Vangl1 | ENSMUSG00000027860 | 0.600 |
| chr3 | 149018000 | 149019000 | 515708 | Gm5149 | ENSMUSG00000069803 | 0.894 |
| chr3 | 98205000 | 98206000 | 464993 | Zfp697 | ENSMUSG00000050064 | 0.830 |
| chr3 | 130829000 | 130830000 | 497568 | Lef1 | ENSMUSG00000027985 | 0.973 |
| chr3 | 99341000 | 99342000 | 466129 | M6pr-ps | ENSMUSG00000078549, | 0.648 |
| | | | | | ENSMUSG00000080832 | |
| chr3 | 154140000 | 154141000 | 520830 | Lhx8 | ENSMUSG00000028201 | 0.489 |
| chr3 | 68330000 | 68331000 | 435118 | Schip1 | ENSMUSG00000027777 | 0.540 |
| chr3 | 50817000 | 50818000 | 417605 | Slc7a11 | ENSMUSG00000027737 | 0.973 |
| chr3 | 152572000 | 152573000 | 519262 | Pigk | ENSMUSG00000039047 | 0.655 |
| chr3 | 159417000 | 159418000 | 526107 | Rpe65 | ENSMUSG00000028174 | 0.887 |
| chr3 | 96723000 | 96724000 | 463511 | Gpr89 | ENSMUSG00000028096 | 0.780 |
| chr3 | 97116000 | 97117000 | 463904 | Bcl9 | ENSMUSG00000038256 | 0.519 |
| chr3 | 38101000 | 38102000 | 404942 | | ENSMUSG00000064315 | 0.211 |
| chr3 | 149189000 | 149190000 | 515879 | Gm5149 | ENSMUSG00000069803 | 0.979 |
| chr3 | 45185000 | 45186000 | 412022 | Pcdh10 | ENSMUSG00000049100 | 0.035 |
| chr3 | 102460000 | 102461000 | 469248 | Ngf | ENSMUSG00000027859 | 0.781 |
| chr3 | 51629000 | 51630000 | 418417 | Maml3 | ENSMUSG00000061143 | 0.978 |
| chr3 | 96493000 | 96494000 | 463281 | Ankrd35 | ENSMUSG00000038354 | 0.385 |
| chr3 | 129255000 | 129256000 | 495994 | Elovl6 | ENSMUSG00000041220 | 0.201 |
| chr3 | 44165000 | 44166000 | 411002 | D3Ertd751e | A,ENSMUSG00000025766 | 0.990 |
| chr3 | 130507000 | 130508000 | 497246 | Rpl34 | ENSMUSG00000062006 | 0.366 |
| chr3 | 130921000 | 130922000 | 497660 | Lef1 | ENSMUSG00000027985 | 0.380 |
| chr3 | 153483000 | 153484000 | 520173 | | ENSMUSG00000062046 | 0.968 |
| chr3 | 96332000 | 96333000 | 463120 | Hfe2 | ENSMUSG00000038403 | 0.566 |
| chr3 | 41372000 | 41373000 | 408209 | Phf17 | ENSMUSG00000025764 | 0.980 |
| chr3 | 68780000 | 68781000 | 435568 | | ENSMUSG00000046999 | 0.969 |
| chr3 | 63843000 | 63844000 | 430631 | Gmps | ENSMUSG00000027823 | 0.061 |
| chr3 | 41391000 | 41392000 | 408228 | Phf17 | ENSMUSG00000025764 | 0.096 |
| chr3 | 68524000 | 68525000 | 435312 | Il12a | ENSMUSG00000027776 | 0.614 |
| chr3 | 8717000 | 8718000 | 375607 | Hey1 | ENSMUSG00000040289 | 0.114 |
| chr3 | 43890000 | 43891000 | 410727 | D3Ertd751e | A,ENSMUSG00000025766 | 0.975 |
| chr3 | 53171000 | 53172000 | 419959 | Lhfp | ENSMUSG00000048332 | 0.781 |
| chr3 | 51163000 | 51164000 | 417951 | Elf2 | ENSMUSG00000037174 | 0.124 |
| chr3 | 51001000 | 51002000 | 417789 | Slc7a11 | ENSMUSG00000027737 | 0.578 |
| chr3 | 102264000 | 102265000 | 469052 | Vangl1 | ENSMUSG00000027860 | 0.600 |
| chr4 | 109103000 | 109104000 | 632057 | Ttc39a | ENSMUSG00000028555 | 0.531 |
| chr4 | 71043000 | 71044000 | 594086 | | ENSMUSG00000061903, | 1.000 |
| | | | | | ENSMUSG00000083914 | |
| chr4 | 62267000 | 62268000 | 585310 | Rgs3 | ENSMUSG00000059810 | 0.536 |
| chr4 | 116947000 | 116948000 | 639901 | Tmem53 | ENSMUSG00000048772 | 0.968 |
| chr4 | 82154000 | 82155000 | 605197 | Nfib | ENSMUSG00000008575 | 0.614 |
| chr4 | 47445000 | 47446000 | 570636 | Tgfbr1 | ENSMUSG00000007613 | 0.968 |
| chr4 | 116828000 | 116829000 | 639782 | Rps8 | ENSMUSG00000047675, | 0.077 |
| | | | | | ENSMUSG00000064457 | |
| chr4 | 113690000 | 113691000 | 636644 | Skint5 | ENSMUSG00000078598 | 0.655 |
| chr4 | 138656000 | 138657000 | 661461 | Nbl1 | ENSMUSG00000041120 | 0.982 |
| chr4 | 137949000 | 137950000 | 660754 | Cda | ENSMUSG00000028755 | 0.707 |
| chr4 | 47398000 | 47399000 | 570589 | Tgfbr1 | ENSMUSG00000007613 | 0.977 |
| chr4 | 106926000 | 106927000 | 629880 | Hspb11 | ENSMUSG00000028617, | 0.031 |
| | | | | | ENSMUSG00000063172 | |
| chr4 | 154374000 | 154375000 | 676931 | Pank4 | ENSMUSG00000029056 | 0.640 |
| chr4 | 116976000 | 116977000 | 639930 | Rnf220 | ENSMUSG00000028677 | 0.473 |
| chr4 | 137307000 | 137308000 | 660112 | Rap1gap | ENSMUSG00000041351 | 0.347 |
| chr4 | 116951000 | 116952000 | 639905 | Tmem53 | ENSMUSG00000048772 | 0.893 |
| chr4 | 138649000 | 138650000 | 661454 | Nbl1 | ENSMUSG00000041120 | 0.474 |
| chr4 | 115825000 | 115826000 | 638779 | Pomgnt1 | ENSMUSG00000028700 | 0.984 |
| chr4 | 149287000 | 149288000 | 671844 | Spsb1 | ENSMUSG00000039911 | 0.584 |
| chr4 | 47014000 | 47015000 | 570205 | Gabbr2 | ENSMUSG00000039809 | 0.492 |
| chr4 | 153893000 | 153894000 | 676450 | Arhgef16 | ENSMUSG00000029032 | 0.043 |
| chr4 | 116985000 | 116986000 | 639939 | Rnf220 | ENSMUSG00000028677 | 0.602 |
| chr4 | 62847000 | 62848000 | 585890 | Kif12 | ENSMUSG00000028357 | 0.105 |
| chr4 | 141376000 | 141377000 | 664181 | Casp9 | ENSMUSG00000028914 | 0.976 |
| chr4 | 119963000 | 119964000 | 642917 | Foxo6 | ENSMUSG00000052135 | 0.492 |
| chr4 | 52456000 | 52457000 | 575647 | Smc2 | ENSMUSG00000028312 | 0.971 |
| chr4 | 137218000 | 137219000 | 660023 | Usp48 | ENSMUSG00000043411 | 0.593 |
| chr4 | 46837000 | 46838000 | 570028 | Gabbr2 | ENSMUSG00000039809 | 0.344 |
| chr4 | 140221000 | 140222000 | 663026 | Arhgef101 | ENSMUSG00000040964 | 0.582 |
| chr4 | 150263000 | 150264000 | 672820 | Errfi1 | ENSMUSG00000028967 | 0.589 |
| chr4 | 46606000 | 46607000 | 569797 | Coro2a | ENSMUSG00000028337 | 0.654 |
| chr4 | 138060000 | 138061000 | 660865 | Camk2n1 | ENSMUSG00000046447 | 0.536 |
| chr4 | 155029000 | 155030000 | 677586 | Mmp23 | ENSMUSG00000029061 | 0.178 |
| chr4 | 107243000 | 107244000 | 630197 | Glis1 | ENSMUSG00000034762 | 0.548 |
| chr4 | 150514000 | 150515000 | 673071 | Camta1 | ENSMUSG00000014592 | 0.114 |
| chr5 | 44595000 | 44596000 | 718679 | Prom1 | ENSMUSG00000029086 | 0.606 |
| chr5 | 66887000 | 66888000 | 740971 | Apbb2 | ENSMUSG00000029207 | 0.972 |
| chr5 | 122493000 | 122494000 | 796432 | | ENSMUSG00000072641 | 0.994 |
| chr5 | 116454000 | 116455000 | 790393 | Cit | ENSMUSG00000029516 | 0.706 |
| chr5 | 116427000 | 116428000 | 790366 | Cit | ENSMUSG00000029516 | 0.614 |
| chr5 | 110977000 | 110978000 | 784951 | Galnt9 | ENSMUSG00000033316 | 0.519 |
| chr5 | 110987000 | 110988000 | 784961 | Galnt9 | ENSMUSG00000033316 | 0.106 |
| chr5 | 146283000 | 146284000 | 819726 | Cyp3a16 | ENSMUSG00000038656 | 0.781 |
| chr5 | 140407000 | 140408000 | 814100 | Elfn1 | ENSMUSG00000048988 | 0.517 |
| chr5 | 151234000 | 151235000 | 824622 | Fry | ENSMUSG00000056602 | 0.975 |
| chr5 | 66886000 | 66887000 | 740970 | Apbb2 | ENSMUSG00000029207 | 0.613 |
| chr5 | 24096000 | 24097000 | 699235 | Chpf2 | ENSMUSG00000038181 | 0.538 |
| chr5 | 140986000 | 140987000 | 814679 | Chst12 | ENSMUSG00000036599 | 0.516 |
| chr5 | 140449000 | 140450000 | 814142 | Elfn1 | ENSMUSG00000048988 | 0.514 |
| chr5 | 74283000 | 74284000 | 748367 | Spata18 | ENSMUSG00000029155 | 0.598 |
| chr5 | 38746000 | 38747000 | 712830 | Drd5 | ENSMUSG00000039358 | 0.975 |
| chr5 | 125772000 | 125773000 | 799620 | Ncor2 | ENSMUSG00000029478 | 0.968 |
| chr5 | 75642000 | 75643000 | 749715 | Pdgfra | ENSMUSG00000029231 | 0.974 |
| chr5 | 75356000 | 75357000 | 749429 | Gm6116 | ENSMUSG00000072874 | 0.380 |
| chr5 | 66444000 | 66445000 | 740528 | | ENSMUSG00000054598 | 0.975 |
| chr5 | 66141000 | 66142000 | 740225 | Pds5a | ENSMUSG00000029202 | 0.968 |
| chr5 | 128822000 | 128823000 | 802670 | Glt1d1 | ENSMUSG00000049971 | 0.707 |
| chr5 | 75544000 | 75545000 | 749617 | Gsx2 | ENSMUSG00000035946 | 0.089 |
| chr5 | 29591000 | 29592000 | 703830 | Rnf32 | ENSMUSG00000029130 | 0.968 |
| chr5 | 148458000 | 148459000 | 821851 | Pan3 | ENSMUSG00000029647 | 0.117 |
| chr5 | 135031000 | 135032000 | 808854 | Clip2 | ENSMUSG00000063146 | 0.027 |
| chr5 | 147572000 | 147573000 | 820965 | Gpr12 | ENSMUSG00000041468 | 0.971 |
| chr5 | 125751000 | 125752000 | 799599 | Ncor2 | ENSMUSG00000029478 | 0.592 |
| chr5 | 112852000 | 112853000 | 786826 | Asphd2 | ENSMUSG00000029348 | 0.516 |
| chr5 | 116048000 | 116049000 | 789987 | Gcn1l1 | ENSMUSG00000041638 | 0.980 |
| chr5 | 71808000 | 71809000 | 745892 | Gabra2 | ENSMUSG00000000560 | 0.894 |
| chr5 | 129288000 | 129289000 | 803130 | Piwil1 | ENSMUSG00000029423 | 0.657 |
| chr5 | 74256000 | 74257000 | 748340 | Spata18 | ENSMUSG00000029155 | 0.571 |
| chr5 | 8930000 | 8931000 | 684118 | Abcb4 | ENSMUSG00000042476 | 0.970 |
| chr5 | 36741000 | 36742000 | 710905 | Sorcs2 | ENSMUSG00000029093 | 0.129 |
| chr6 | 113592000 | 113593000 | 936418 | Irak2 | ENSMUSG00000060477 | 0.612 |
| chr6 | 35312000 | 35313000 | 858188 | Fam180a | ENSMUSG00000047420 | 0.645 |
| chr6 | 113622000 | 113623000 | 936448 | Irak2 | ENSMUSG00000060477 | 0.646 |
| chr6 | 93644000 | 93645000 | 916470 | | ENSMUSG00000077180 | 0.984 |
| chr6 | 71485000 | 71486000 | 894311 | Rnf103 | ENSMUSG00000052656 | 0.976 |
| chr6 | 56967000 | 56968000 | 879793 | V1rc20 | ENSMUSG00000058923 | 0.646 |
| chr6 | 114459000 | 114460000 | 93728 | 5Hrh1 | ENSMUSG00000053004 | 0.606 |
| chr6 | 52152000 | 52153000 | 874978 | Hoxa3 | ENSMUSG00000079560 | 0.894 |
| chr6 | 114167000 | 114168000 | 936993 | Slc6a11 | ENSMUSG00000030307 | 0.506 |
| chr6 | 52140000 | 52141000 | 874966 | Hoxa3 | ENSMUSG00000079560 | 0.575 |
| chr6 | 120083000 | 120084000 | 942909 | Ninj2 | ENSMUSG00000041377 | 0.981 |
| chr6 | 114576000 | 114577000 | 937402 | Hrh1 | ENSMUSG00000053004 | 0.655 |
| chr6 | 91642000 | 91643000 | 914468 | S1c6a6 | ENSMUSG00000030096 | 0.974 |
| chr6 | 113892000 | 113893000 | 936718 | Atp2b2 | ENSMUSG00000030302 | 0.619 |
| chr6 | 115569000 | 115570000 | 938395 | Mkrn2 | ENSMUSG00000000439 | 0.147 |
| chr6 | 88868000 | 88869000 | 911694 | Tpra1 | ENSMUSG00000002871 | 0.538 |
| chr6 | 121007000 | 121008000 | 943833 | | ENSMUSG00000052437 | 0.984 |
| chr6 | 93016000 | 93017000 | 915842 | Adamts9 | ENSMUSG00000030022 | 0.184 |
| chr6 | 55531000 | 55532000 | 878357 | Adcyap1r1 | ENSMUSG00000029778 | 0.659 |
| chr6 | 120015000 | 120016000 | 942841 | Wnk1 | ENSMUSG00000045962 | 0.612 |
| chr6 | 121857000 | 121858000 | 944683 | Mug1 | ENSMUSG00000059908 | 0.641 |
| chr6 | 120062000 | 120063000 | 942888 | Ninj2 | ENSMUSG00000041377 | 0.089 |
| chr6 | 71930000 | 71931000 | 894756 | Polr1a | ENSMUSG00000049553 | 0.581 |
| chr6 | 113233000 | 113234000 | 936059 | Cpne9 | ENSMUSG00000030270 | 0.055 |
| chr6 | 119270000 | 119271000 | 942096 | Cacna2d4 | ENSMUSG00000041460 | 0.509 |
| chr6 | 95698000 | 95699000 | 918524 | Suc1g2 | ENSMUSG00000061838 | 0.968 |
| chr6 | 119076000 | 119077000 | 941902 | Cacna1c | ENSMUSG00000051331 | 0.980 |
| chr6 | 114478000 | 114479000 | 937304 | Hrh1 | ENSMUSG00000053004 | 0.595 |
| chr6 | 120922000 | 120923000 | 943748 | Bid | ENSMUSG00000004446 | 0.970 |
| chr6 | 90569000 | 90570000 | 913395 | S1c41a3 | ENSMUSG00000030089 | 0.536 |
| chr6 | 37476000 | 37477000 | 860352 | Creb312 | ENSMUSG00000038648 | 0.567 |
| chr6 | 92560000 | 92561000 | 915386 | Prickle2 | ENSMUSG00000030020 | 0.622 |
| chr6 | 133994000 | 133995000 | 956820 | Etv6 | ENSMUSG00000030199 | 0.275 |
| chr6 | 97236000 | 97237000 | 920062 | Lmod3 | ENSMUSG00000044086 | 0.970 |
| chr6 | 114568000 | 114569000 | 937394 | Hrh1 | ENSMUSG00000053004 | 0.587 |
| chr7 | 63706000 | 63707000 | 1025546 | Oca2 | ENSMUSG00000030450 | 0.578 |
| chr7 | 148203000 | 148204000 | 1109860 | Ifitm6 | ENSMUSG00000059108 | 0.255 |
| chr7 | 80664000 | 80665000 | 1042454 | Chd2 | ENSMUSG00000025788 | 0.973 |
| chr7 | 29529000 | 29530000 | 998369 | Sars2 | ENSMUSG00000070699 | 0.977 |
| chr7 | 150661000 | 150662000 | 1112279 | Slc22a18 | ENSMUSG00000000154 | 0.559 |
| chr7 | 28261000 | 28262000 | 997101 | Sertad3 | ENSMUSG00000055200 | 0.978 |
| chr7 | 138081000 | 138082000 | 1099817 | Htra1 | ENSMUSG00000006205 | 0.487 |
| chr7 | 86133000 | 86134000 | 1047923 | Isg20 | ENSMUSG00000039236 | 0.977 |
| chr7 | 25919000 | 25920000 | 994759 | Pou2f2 | ENSMUSG00000008496 | 0.512 |
| chr7 | 135532000 | 135533000 | 1097268 | BC017158 | ENSMUSG00000030780 | 0.575 |
| chr7 | 139909000 | 139910000 | 1101595 | Lhpp | ENSMUSG00000030946 | 0.566 |
| chr7 | 64394000 | 64395000 | 1026234 | Gabrg3 | ENSMUSG00000055026 | 0.653 |
| chr7 | 31251000 | 31252000 | 1000091 | Nphs1 | ENSMUSG00000006649 | 0.115 |
| chr7 | 137155000 | 137156000 | 1098891 | Brwd2 | ENSMUSG00000042055 | 0.564 |
| chr7 | 30000000 | 30001000 | 998840 | Catsperg1 | ENSMUSG00000049676 | 0.539 |
| chr7 | 30010000 | 30011000 | 998850 | Catsperg1 | ENSMUSG00000049676 | 0.579 |
| chr7 | 52120000 | 52121000 | 1013960 | Pnkp | ENSMUSG00000002963 | 0.510 |
| chr7 | 134528000 | 134529000 | 1096264 | Zfp747 | ENSMUSG00000054381 | 0.968 |
| chr7 | 29957000 | 29958000 | 998797 | Ggn | ENSMUSG00000031493 | 0.652 |
| chr7 | 118165000 | 118166000 | 1079901 | Mrvi1 | ENSMUSG00000005611 | 0.556 |
| chr7 | 80522000 | 80523000 | 1042312 | Rgma | ENSMUSG00000070509 | 0.541 |
| chr7 | 142677000 | 142678000 | 1104363 | Foxi2 | ENSMUSG00000048377 | 0.104 |
| chr7 | 26388000 | 26389000 | 995228 | Ceacam2 | ENSMUSG00000054385 | 0.968 |
| chr7 | 53048000 | 53049000 | 1014888 | Lmtk3 | ENSMUSG00000062044 | 0.658 |
| chr7 | 52679000 | 52680000 | 1014519 | Lhb | ENSMUSG00000038194 | 0.968 |
| chr7 | 25941000 | 25942000 | 994781 | | ENSMUSG00000074274 | 0.489 |
| chr7 | 127450000 | 127451000 | 1089186 | Abca14 | ENSMUSG00000062017 | 0.969 |
| chr7 | 148124000 | 148125000 | 1109781 | Nlrp6 | ENSMUSG00000038745 | 0.579 |
| chr7 | 148031000 | 148032000 | 1109688 | Scgb1c1 | ENSMUSG00000038801 | 0.362 |
| chr7 | 72838000 | 72839000 | 1034628 | Tm2d3 | ENSMUSG00000078681 | 0.031 |
| chr7 | 36472000 | 36473000 | 1005312 | Pdcd5 | ENSMUSG00000030417 | 0.213 |
| chr7 | 52615000 | 52616000 | 1014455 | Ppfia3 | ENSMUSG00000003863 | 0.525 |
| chr7 | 30719000 | 30720000 | 999559 | Zfp27 | ENSMUSG00000062040 | 0.981 |
| chr7 | 52128000 | 52129000 | 1013968 | Ptov1 | ENSMUSG00000038502 | 0.585 |
| chr7 | 92172000 | 92173000 | 1053957 | Vmn2r66 | ENSMUSG00000072241 | 0.893 |
| chr8 | 119062000 | 119063000 | 1226266 | Dynlrb2 | ENSMUSG00000034467 | 0.591 |
| chr8 | 24265000 | 24266000 | 1133309 | Nkx6-3 | ENSMUSG00000063672 | 0.582 |
| chr8 | 119147000 | 119148000 | 1226351 | Cdyl2 | ENSMUSG00000031758 | 0.969 |
| chr8 | 18034000 | 18035000 | 1129177 | Csmd1 | ENSMUSG00000060924 | 0.781 |
| chr8 | 116490000 | 116491000 | 1223694 | Adamts18 | ENSMUSG00000053399 | 0.609 |
| chr8 | 119154000 | 119155000 | 1226358 | Cdyl2 | ENSMUSG00000031758 | 0.496 |
| chr8 | 107998000 | 107999000 | 1215202 | Tppp3 | ENSMUSG00000014846 | 0.554 |
| chr8 | 25462000 | 25463000 | 1134506 | | ENSMUSG00000053979 | 0.186 |
| chr8 | 11605000 | 11606000 | 1122748 | Ing1 | ENSMUSG00000045969 | 0.969 |
| chr8 | 109135000 | 109136000 | 1216339 | Cdh1 | ENSMUSG00000000303 | 0.596 |
| chr8 | 117689000 | 117690000 | 1224893 | Wwox | ENSMUSG00000004637 | 0.077 |
| chr8 | 109576000 | 109577000 | 1216780 | Pdf | ENSMUSG00000078931 | 0.971 |
| chr8 | 11476000 | 11477000 | 1122619 | Col4a2 | ENSMUSG00000031503 | 0.048 |
| chr8 | 28267000 | 28268000 | 1137311 | Brf2 | ENSMUSG00000031487 | 0.969 |
| chr8 | 8319000 | 8320000 | 1119462 | | ENSMUSG00000077378 | 0.979 |
| chr8 | 109363000 | 109364000 | 1216567 | Tmco7 | ENSMUSG00000041949 | 0.581 |
| chr8 | 117268000 | 117269000 | 1224472 | Wwox | ENSMUSG00000004637 | 0.496 |
| chr8 | 16794000 | 16795000 | 1127937 | Csmd1 | ENSMUSG00000060924 | 0.980 |
| chr8 | 109034000 | 109035000 | 1216238 | Cdh3 | ENSMUSG00000061048 | 0.036 |
| chr8 | 26081000 | 26082000 | 1135125 | Adam32 | ENSMUSG00000037437 | 0.974 |
| chr8 | 117123000 | 117124000 | 1224327 | Wwox | ENSMUSG00000004637 | 0.645 |
| chr8 | 124847000 | 124848000 | 1232051 | Zfpm1 | ENSMUSG00000049577 | 0.641 |
| chr8 | 117231000 | 117232000 | 1224435 | Wwox | ENSMUSG00000004637 | 0.344 |
| chr8 | 109202000 | 109203000 | 1216406 | Cdh1 | ENSMUSG00000000303 | 0.106 |
| chr8 | 15029000 | 15030000 | 1126172 | Kbtbd11 | ENSMUSG00000055675 | 0.510 |
| chr8 | 18751000 | 18752000 | 1129894 | Angpt2 | ENSMUSG00000031465 | 0.978 |
| chr8 | 11464000 | 11465000 | 1122607 | Col4a2 | ENSMUSG00000031503 | 0.591 |
| chr8 | 11421000 | 11422000 | 1122564 | Col4a2 | ENSMUSG00000031503 | 0.646 |
| chr8 | 114534000 | 114535000 | 1221738 | Kars | ENSMUSG00000031948 | 0.000 |
| chr8 | 119606000 | 119607000 | 1226810 | Pkd1l2 | ENSMUSG00000034416 | 0.647 |
| chr8 | 19090000 | 19091000 | 1130233 | Defb39 | ENSMUSG00000061847 | 0.795 |
| chr8 | 12467000 | 12468000 | 1123610 | Gm5607 | ENSMUSG00000047935 | 0.532 |
| chr8 | 108693000 | 108694000 | 1215897 | Slc7a6 | ENSMUSG00000031904 | 0.043 |
| chr8 | 124579000 | 124580000 | 1231783 | Banp | ENSMUSG00000025316 | 0.662 |
| chr8 | 125039000 | 125040000 | 1232243 | Fam38a | ENSMUSG00000014444 | 0.973 |
| chr9 | 64478000 | 64479000 | 1300320 | Megfl1 | ENSMUSG00000036466 | 0.780 |
| chr9 | 5029000 | 5030000 | 1240972 | Gria4 | ENSMUSG00000025892 | 0.993 |
| chr9 | 30371000 | 30372000 | 1266263 | Snx19 | ENSMUSG00000031993 | 0.616 |
| chr9 | 14477000 | 14478000 | 1250369 | Amotl1 | ENSMUSG00000013076 | 0.830 |
| chr9 | 20712000 | 20713000 | 1256604 | Eif3g | ENSMUSG00000070319 | 0.969 |
| chr9 | 20548000 | 20549000 | 1256440 | Olfm2 | ENSMUSG00000032172 | 0.183 |
| chr9 | 78369000 | 78370000 | 1314211 | Eef1a1 | ENSMUSG00000037742 | 0.060 |
| chr9 | 71465000 | 71466000 | 1307307 | Gcom1 | ENSMUSG00000041361 | 0.588 |
| chr9 | 98765000 | 98766000 | 1334495 | | ENSMUSG00000032460 | 0.488 |
| chr9 | 54281000 | 54282000 | 1290123 | Dmxl2 | ENSMUSG00000041268 | 0.697 |
| chr9 | 119542000 | 119543000 | 1355198 | Scn5a | ENSMUSG00000032511 | 0.533 |
| chr9 | 26749000 | 26750000 | 1262641 | Gm1110 | ENSMUSG00000079644 | 0.548 |
| chr9 | 27108000 | 27109000 | 1263000 | Igsf9b | ENSMUSG00000034275 | 0.037 |
| chr9 | 100740000 | 100741000 | 1336470 | Stag1 | ENSMUSG00000037286 | 0.648 |
| chr9 | 3199000 | 3200000 | 1239142 | | ENSMUSG00000042360 | 0.337 |
| chr9 | 87134000 | 87135000 | 1322886 | | ENSMUSG00000056919 | 0.970 |
| chr9 | 46251000 | 46252000 | 1282142 | | ENSMUSG00000056617 | 0.035 |
| chr9 | 107803000 | 107804000 | 1343525 | Mon1a | ENSMUSG00000032583 | 0.242 |
| chr9 | 119441000 | 119442000 | 1355097 | Exog | ENSMUSG00000042787 | 0.659 |
| chr9 | 23786000 | 23787000 | 1259678 | Bmper | ENSMUSG00000031963 | 0.780 |
| chr9 | 99010000 | 99011000 | 1334740 | Gm1123 | ENSMUSG00000044860 | 0.602 |
| chr9 | 119469000 | 119470000 | 1355125 | Exog | ENSMUSG00000042787 | 0.610 |
| chr9 | 63818000 | 63819000 | 1299660 | Smad3 | ENSMUSG00000032402 | 0.546 |
| chr9 | 21905000 | 21906000 | 1257797 | Cnn1 | ENSMUSG00000001349 | 0.547 |
| chr9 | 86648000 | 86649000 | 1322401 | Prss35 | ENSMUSG00000033491 | 0.968 |
| chr9 | 60719000 | 60720000 | 1296561 | | ENSMUSG00000052143 | 0.980 |
| chr9 | 59450000 | 59451000 | 1295292 | Brunol6 | ENSMUSG00000032297 | 0.365 |
| chr9 | 57505000 | 57506000 | 1293347 | Cyp1a1 | ENSMUSG00000032315 | 0.661 |
| chr9 | 121210000 | 121211000 | 1356866 | Trak1 | ENSMUSG00000032536 | 0.662 |
| chr9 | 11634000 | 11635000 | 1247577 | | ENSMUSG00000077550 | 0.975 |
| chr9 | 49014000 | 49015000 | 1284905 | Tmprss5 | ENSMUSG00000032268 | 0.391 |
| chr9 | 17002000 | 17003000 | 1252894 | Fat3 | ENSMUSG00000074505 | 0.602 |
| chr9 | 119508000 | 119509000 | 1355164 | Scn5a | ENSMUSG00000032511 | 0.411 |
| chr9 | 99371000 | 99372000 | 1335101 | | ENSMUSG00000046242 | 0.581 |
| chr9 | 76105000 | 76106000 | 1311947 | Gfral | ENSMUSG00000059383 | 0.556 |
| chr10 | 85249000 | 85250000 | 1441793 | Btbd11 | ENSMUSG00000020042 | 0.655 |
| chr10 | 75416000 | 75417000 | 1431960 | Vpreb3 | ENSMUSG00000000903 | 0.616 |
| chr10 | 51662000 | 51663000 | 1408296 | | ENSMUSG00000062224 | 0.894 |
| chr10 | 115215000 | 115216000 | 1471759 | LgrS | ENSMUSG00000020140 | 0.363 |
| chr10 | 83855000 | 83856000 | 1440399 | Appl2 | ENSMUSG00000020263 | 0.254 |
| chr10 | 90735000 | 90736000 | 1447279 | Tmpo | ENSMUSG00000019961 | 0.548 |
| chr10 | 117325000 | 117326000 | 1473869 | Rap1b | ENSMUSG00000052681 | 0.573 |
| chr10 | 75345000 | 75346000 | 1431889 | Mif | ENSMUSG00000033307 | 0.549 |
| chr10 | 85194000 | 85195000 | 1441738 | Btbd11 | ENSMUSG00000020042 | 0.619 |
| chr10 | 44176000 | 44177000 | 1400810 | Atg5 | ENSMUSG00000038160 | 0.476 |
| chr10 | 76133000 | 76134000 | 1432677 | Col6a2 | ENSMUSG00000020241 | 0.588 |
| chr10 | 92841000 | 92842000 | 1449385 | Elk3 | ENSMUSG00000008398 | 0.975 |
| chr10 | 94048000 | 94049000 | 1450592 | Tmcc3 | ENSMUSG00000020023 | 0.970 |
| chr10 | 84220000 | 84221000 | 1440764 | Rfx4 | ENSMUSG00000020037 | 0.211 |
| chr10 | 118113000 | 118114000 | 1474657 | Ifng | ENSMUSG00000055170 | 0.600 |
| chr10 | 45400000 | 45401000 | 1402034 | Hace1 | ENSMUSG00000038822 | 0.977 |
| chr10 | 111079000 | 111080000 | 1467623 | Phlda1 | ENSMUSG00000020205 | 0.973 |
| chr10 | 92739000 | 92740000 | 1449283 | Cdk17 | ENSMUSG00000020015 | 0.385 |
| chr10 | 82467000 | 82468000 | 1439011 | Chst11 | ENSMUSG00000034612 | 0.107 |
| chr10 | 93294000 | 93295000 | 1449838 | Usp44 | ENSMUSG00000020020 | 0.341 |
| chr10 | 80415000 | 80416000 | 1436959 | Gadd45b | ENSMUSG00000015312 | 0.644 |
| chr10 | 92997000 | 92998000 | 1449541 | Hal | ENSMUSG00000020017 | 0.055 |
| chr10 | 83995000 | 83996000 | 1440539 | | ENSMUSG00000020033 | 0.337 |
| chr10 | 42742000 | 42743000 | 1399376 | Scml4 | ENSMUSG00000044770 | 0.181 |
| chr10 | 76421000 | 76422000 | 1432965 | Col6a1 | ENSMUSG00000001119, | 0.975 |
| | | | | | ENSMUSG00000078445 | |
| chr10 | 70862000 | 70863000 | 1427406 | Ipmk | ENSMUSG00000060733 | 0.404 |
| chr10 | 44149000 | 44150000 | 1400783 | Atg5 | ENSMUSG00000038160 | 0.187 |
| chr10 | 6199000 | 6200000 | 1362882 | Akap12 | ENSMUSG00000038587 | 0.973 |
| chr10 | 115629000 | 115630000 | 1472173 | Ptprr | ENSMUSG00000020151 | 0.604 |
| chr10 | 80291000 | 80292000 | 1436835 | Oaz1 | ENSMUSG00000035242 | 0.547 |
| chr10 | 42639000 | 42640000 | 1399273 | Scml4 | ENSMUSG00000044770 | 0.972 |
| chr10 | 83854000 | 83855000 | 1440398 | Appl2 | ENSMUSG00000020263 | 0.366 |
| chr10 | 93508000 | 93509000 | 1450052 | Fgd6 | ENSMUSG00000020021 | 0.969 |
| chr10 | 59002000 | 59003000 | 1415551 | Ccdc109a | ENSMUSG00000009647 | 0.574 |
| chr10 | 58540000 | 58541000 | 1415089 | Sh3rf3 | ENSMUSG00000037990 | 0.572 |
| chr11 | 4029000 | 4030000 | 1487567 | Sec1412 | ENSMUSG00000003585 | 0.968 |
| chr11 | 45926000 | 45927000 | 1529414 | Adam19 | ENSMUSG00000011256 | 0.981 |
| chr11 | 106891000 | 106892000 | 1590329 | | ENSMUSG00000078607 | 0.494 |
| chr11 | 117984000 | 117985000 | 1601422 | Dnahc17 | ENSMUSG00000033987 | 0.649 |
| chr11 | 48650000 | 48651000 | 1532138 | Trim7 | ENSMUSG00000040350 | 0.502 |
| chr11 | 66988000 | 66989000 | 1550476 | Myh2 | ENSMUSG00000033196 | 0.986 |
| chr11 | 75765000 | 75766000 | 1559253 | Rph3al | ENSMUSG00000020847 | 0.969 |
| chr11 | 75450000 | 75451000 | 1558938 | Inpp5k | ENSMUSG00000006127 | 0.214 |
| chr11 | 69666000 | 69667000 | 1553154 | Plscr3 | ENSMUSG00000019461 | 0.780 |
| chr11 | 65271000 | 65272000 | 1548759 | Myocd | ENSMUSG00000020542 | 0.978 |
| chr11 | 61115000 | 61116000 | 1544603 | Aldh3a2 | ENSMUSG00000010025 | 0.524 |
| chr11 | 67489000 | 67490000 | 1550977 | Gas7 | ENSMUSG00000033066 | 0.278 |
| chr11 | 68767000 | 68768000 | 1552255 | Arhgef15 | ENSMUSG00000052921 | 0.201 |
| chr11 | 3404000 | 3405000 | 1486942 | Inpp5j | ENSMUSG00000034570 | 0.591 |
| chr11 | 69218000 | 69219000 | 1552706 | Tmem88 | ENSMUSG00000045377 | 0.968 |
| chr11 | 45870000 | 45871000 | 1529358 | Adam19 | ENSMUSG00000011256 | 0.043 |
| chr11 | 48982000 | 48983000 | 1532470 | Olfr1394 | ENSMUSG00000048378 | 0.088 |
| chr11 | 61166000 | 61167000 | 1544654 | Slc47a2 | ENSMUSG00000069855 | 0.650 |
| chr11 | 3578000 | 3579000 | 1487116 | Morc2a | ENSMUSG00000034543 | 0.977 |
| chr11 | 96207000 | 96208000 | 1579645 | Hoxb3 | ENSMUSG00000048763 | 0.655 |
| chr11 | 121247000 | 121248000 | 1604685 | Wdr451 | ENSMUSG00000025173 | 0.591 |
| chr11 | 32129000 | 32130000 | 1515667 | Mpg | ENSMUSG00000020287 | 0.985 |
| chr11 | 70029000 | 70030000 | 1553517 | Slc16a11 | ENSMUSG00000040938 | 0.473 |
| chr11 | 69831000 | 69832000 | 1553319 | Dlg4 | ENSMUSG00000020886 | 0.516 |
| chr11 | 67611000 | 67612000 | 1551099 | Dhrs7c | ENSMUSG00000033044 | 0.707 |
| chr11 | 61891000 | 61892000 | 1545379 | Cytsb | ENSMUSG00000042331 | 0.027 |
| chr11 | 65240000 | 65241000 | 1548728 | Myocd | ENSMUSG00000020542 | 0.983 |
| chr11 | 115195000 | 115196000 | 1598633 | Otop2 | ENSMUSG00000050201 | 0.143 |
| chr11 | 73078000 | 73079000 | 1556566 | Trpv1 | ENSMUSG00000005952 | 0.655 |
| chr11 | 77698000 | 77699000 | 1561186 | Myo18a | ENSMUSG00000000631 | 0.615 |
| chr11 | 17184000 | 17185000 | 1500722 | C1d | ENSMUSG00000000581 | 0.561 |
| chr11 | 85104000 | 85105000 | 1568592 | Appbp2 | ENSMUSG00000018481 | 0.970 |
| chr11 | 58948000 | 58949000 | 1542436 | Obscn | ENSMUSG00000061462 | 0.043 |
| chr11 | 32168000 | 32169000 | 1515706 | Mare | ENSMUSG00000020289 | 0.610 |
| chr11 | 117062000 | 117063000 | 1600500 | Sept9 | ENSMUSG00000059248 | 0.546 |
| chr12 | 110498000 | 110499000 | 1711988 | Begain | ENSMUSG00000040867 | 0.970 |
| chr12 | 110272000 | 110273000 | 1711762 | Wdr25 | ENSMUSG00000040877 | 0.616 |
| chr12 | 29768000 | 29769000 | 1631742 | Tssc1 | ENSMUSG00000036613 | 0.577 |
| chr12 | 32516000 | 32517000 | 1634490 | Gpr22 | ENSMUSG00000044067 | 0.983 |
| chr12 | 27219000 | 27220000 | 1629193 | Cmpk2 | ENSMUSG00000020638 | 0.510 |
| chr12 | 106915000 | 106916000 | 1708405 | Bdkrb1 | ENSMUSG00000041347 | 0.985 |
| chr12 | 109577000 | 109578000 | 1711067 | Cyp46a1 | ENSMUSG00000021259 | 0.554 |
| chr12 | 71553000 | 71554000 | 1673143 | Trim9 | ENSMUSG00000021071 | 0.002 |
| chr12 | 109209000 | 109210000 | 1710699 | | ENSMUSG00000060375 | 0.565 |
| chr12 | 77414000 | 77415000 | 1679004 | Mthfd1 | ENSMUSG00000021048 | 0.984 |
| chr12 | 3366000 | 3367000 | 1605648 | Kif3c | ENSMUSG00000020668 | 0.362 |
| chr12 | 16075000 | 16076000 | 1618348 | Trib2 | ENSMUSG00000020601 | 0.973 |
| chr12 | 70859000 | 70860000 | 1672449 | Atp5s | ENSMUSG00000054894 | 0.105 |
| chr12 | 77317000 | 77318000 | 1678907 | Esr2 | ENSMUSG00000021055 | 0.516 |
| chr12 | 106372000 | 106373000 | 1707862 | Glrx5 | ENSMUSG00000021102 | 0.211 |
| chr12 | 111900000 | 111901000 | 1713390 | Dync1h1 | ENSMUSG00000018707 | 0.987 |
| chr12 | 120161000 | 120162000 | 1721651 | Sp8 | ENSMUSG00000048562 | 0.612 |
| chr12 | 12558000 | 12559000 | 1614831 | Fam49a | ENSMUSG00000020589 | 0.554 |
| chr12 | 110309000 | 110310000 | 1711799 | Begain | ENSMUSG00000040867 | 0.132 |
| chr12 | 29483000 | 29484000 | 1631457 | Tssc1 | ENSMUSG00000036613 | 0.610 |
| chr12 | 25412000 | 25413000 | 1627386 | Rrm2 | ENSMUSG00000020649 | 0.585 |
| chr12 | 25595000 | 25596000 | 1627569 | Mboat2 | ENSMUSG00000020646 | 0.984 |
| chr12 | 22990000 | 22991000 | 1625063 | | ENSMUSG00000073164 | 0.117 |
| chr12 | 41126000 | 41127000 | 1643097 | Ifrd1 | ENSMUSG00000001627 | 0.979 |
| chr12 | 105456000 | 105457000 | 1706946 | Serpina3f | ENSMUSG00000066363 | 0.795 |
| chr12 | 70858000 | 70859000 | 1672448 | Atp5s | ENSMUSG00000054894 | 0.160 |
| chr12 | 109189000 | 109190000 | 1710679 | | ENSMUSG00000060375 | 0.527 |
| chr12 | 53846000 | 53847000 | 1655436 | Akap6 | ENSMUSG00000061603 | 0.521 |
| chr12 | 4880000 | 4881000 | 1607153 | | ENSMUSG00000051721 | 0.539 |
| chr12 | 72398000 | 72399000 | 1673988 | | ENSMUSG00000034601 | 0.609 |
| chr12 | 109856000 | 109857000 | 1711346 | Evl | ENSMUSG00000021262 | 0.551 |
| chr12 | 71368000 | 71369000 | 1672958 | Pygl | ENSMUSG00000021069 | 0.477 |
| chr12 | 74638000 | 74639000 | 1676228 | | ENSMUSG00000056359 | 0.588 |
| chr12 | 35345000 | 35346000 | 1637319 | Hdac9 | ENSMUSG00000004698 | 0.510 |
| chr12 | 59370000 | 59371000 | 1660960 | Clec14a | ENSMUSG00000045930 | 0.575 |
| chr13 | 59765000 | 59766000 | 1779334 | Naa35 | ENSMUSG00000021555 | 0.979 |
| chr13 | 76000000 | 76001000 | 1795520 | Glrx | ENSMUSG00000021591 | 0.781 |
| chr13 | 38751000 | 38752000 | 1758369 | Eef1e1 | ENSMUSG00000001707 | 0.343 |
| chr13 | 40990000 | 40991000 | 1760572 | Gcnt2 | ENSMUSG00000021360 | 0.658 |
| chr13 | 77139000 | 77140000 | 1796659 | Mctp1 | ENSMUSG00000021596 | 0.604 |
| chr13 | 49415000 | 49416000 | 1768997 | Fgd3 | ENSMUSG00000037946 | 0.346 |
| chr13 | 56077000 | 56078000 | 1775646 | Pitx1 | ENSMUSG00000021506 | 0.830 |
| chr13 | 82225000 | 82226000 | 1801745 | Cetn3 | ENSMUSG00000021537 | 0.599 |
| chr13 | 55020000 | 55021000 | 1774589 | Tspan17 | ENSMUSG00000025875 | 0.510 |
| chr13 | 43483000 | 43484000 | 1763065 | Sirt5 | ENSMUSG00000054021 | 0.969 |
| chr13 | 54894000 | 54895000 | 1774463 | Tspan17 | ENSMUSG00000025875 | 0.131 |
| chr13 | 95993000 | 95994000 | 1814860 | Pde8b | ENSMUSG00000021684 | 0.061 |
| chr13 | 56101000 | 56102000 | 1775670 | Pitx1 | ENSMUSG00000021506 | 0.664 |
| chr13 | 86771000 | 86772000 | 1806291 | Cox7c | A,ENSMUSG00000017778 | 0.920 |
| chr13 | 53330000 | 53331000 | 1772912 | Nfil3 | ENSMUSG00000056749 | 0.489 |
| chr13 | 48812000 | 48813000 | 1768394 | Barx1 | ENSMUSG00000021381 | 0.697 |
| chr13 | 73397000 | 73398000 | 1792917 | Irx4 | ENSMUSG00000021604 | 0.036 |
| chr13 | 96324000 | 96325000 | 1815191 | F2rl1 | ENSMUSG00000021678 | 0.550 |
| chr13 | 54940000 | 54941000 | 1774509 | Tspan17 | ENSMUSG00000025875 | 0.279 |
| chr13 | 86554000 | 86555000 | 1806074 | Cox7c | A,ENSMUSG00000017778 | 0.920 |
| chr13 | 54925000 | 54926000 | 1774494 | Tspan17 | ENSMUSG00000025875 | 0.116 |
| chr13 | 55274000 | 55275000 | 1774843 | Fgfr4 | ENSMUSG00000005320 | 0.576 |
| chr13 | 55709000 | 55710000 | 1775278 | B4galt7 | ENSMUSG00000021504 | 0.980 |
| chr13 | 100412000 | 100413000 | 1819279 | Mtap1b | ENSMUSG00000052727 | 0.485 |
| chr13 | 73653000 | 73654000 | 1793173 | Lpcat1 | ENSMUSG00000021608 | 0.970 |
| chr13 | 52665000 | 52666000 | 1772247 | Diras2 | ENSMUSG00000047842 | 0.978 |
| chr13 | 117104000 | 117105000 | 1835951 | Isl1 | ENSMUSG00000042258 | 0.030 |
| chr13 | 24788000 | 24789000 | 1744406 | Fam65b | ENSMUSG00000036006 | 0.657 |
| chr13 | 47211000 | 47212000 | 1766793 | Dek | ENSMUSG00000021377 | 0.977 |
| chr13 | 108636000 | 108637000 | 1827483 | Zswim6 | ENSMUSG00000032846 | 0.178 |
| chr13 | 61026000 | 61027000 | 1780595 | Tpbpb | ENSMUSG00000062705 | 0.830 |
| chr13 | 102732000 | 102733000 | 1821599 | Pik3r1 | ENSMUSG00000041417 | 0.968 |
| chr13 | 24954000 | 24955000 | 1744572 | | ENSMUSG00000006711 | 0.619 |
| chr13 | 114100000 | 114101000 | 1832947 | Gzmk | ENSMUSG00000042385 | 0.971 |
| chr13 | 51526000 | 51527000 | 1771108 | S1pr3 | ENSMUSG00000067586 | 0.550 |
| chr14 | 57183000 | 57184000 | 1892761 | Rnf17 | ENSMUSG00000000365 | 0.978 |
| chr14 | 106319000 | 106320000 | 1941897 | Spry2 | ENSMUSG00000022114 | 0.123 |
| chr14 | 105999000 | 106000000 | 1941577 | | ENSMUSG00000022116 | 0.981 |
| chr14 | 56719000 | 56720000 | 1892297 | Mcpt8 | ENSMUSG00000022157 | 0.795 |
| chr14 | 60590000 | 60591000 | 1896168 | Shisa2 | ENSMUSG00000044461 | 0.974 |
| chr14 | 111264000 | 111265000 | 1946842 | Slitrk6 | ENSMUSG00000045871 | 0.580 |
| chr14 | 81960000 | 81961000 | 1917538 | Olfm4 | A,ENSMUSG00000022026 | 0.620 |
| chr14 | 70216000 | 70217000 | 1905794 | Rhobtb2 | ENSMUSG00000022075 | 0.583 |
| chr14 | 57752000 | 57753000 | 1893330 | Gjb6 | ENSMUSG00000040055 | 0.035 |
| chr14 | 32114000 | 32115000 | 1867841 | Bap1 | ENSMUSG00000021901 | 0.968 |
| chr14 | 122033000 | 122034000 | 1957611 | S1c15a1 | ENSMUSG00000025557 | 0.603 |
| chr14 | 121197000 | 121198000 | 1956775 | Rap2a | ENSMUSG00000051615 | 0.618 |
| chr14 | 33421000 | 33422000 | 1869148 | Prrxl1 | ENSMUSG00000041730 | 0.662 |
| chr14 | 81245000 | 81246000 | 1916823 | Olfm4 | A,ENSMUSG00000022026 | 0.620 |
| chr14 | 120198000 | 120199000 | 1955776 | Hs6st3 | ENSMUSG00000053465 | 0.974 |
| chr14 | 73245000 | 73246000 | 1908823 | Fndc3a | ENSMUSG00000033487 | 0.489 |
| chr14 | 119647000 | 119648000 | 1955225 | Hs6st3 | ENSMUSG00000053465 | 0.657 |
| chr14 | 49199000 | 49200000 | 1884777 | | ENSMUSG00000036339 | 0.663 |
| chr14 | 70567000 | 70568000 | 1906145 | | ENSMUSG00000044551 | 0.492 |
| chr14 | 32461000 | 32462000 | 1868188 | Btd | ENSMUSG00000021900 | 0.969 |
| chr14 | 121311000 | 121312000 | 1956889 | IpoS | ENSMUSG00000030662 | 0.000 |
| chr14 | 32930000 | 32931000 | 1868657 | Oxnad1 | ENSMUSG00000021906 | 0.254 |
| chr14 | 56445000 | 56446000 | 1892023 | Nfatc4 | ENSMUSG00000023411 | 0.650 |
| chr14 | 80124000 | 80125000 | 1915702 | Lect1 | ENSMUSG00000022025 | 0.545 |
| chr14 | 122785000 | 122786000 | 1958363 | Clybl | ENSMUSG00000025545 | 0.970 |
| chr14 | 84828000 | 84829000 | 1920406 | Olfm4 | ENSMUSG00000022026 | 0.781 |
| chr14 | 58417000 | 58418000 | 1893995 | Sap18 | ENSMUSG00000021963 | 0.097 |
| chr14 | 47600000 | 47601000 | 1883178 | Samd4 | ENSMUSG00000021838 | 0.566 |
| chr14 | 47833000 | 47834000 | 1883411 | Gch1 | ENSMUSG00000037580 | 0.646 |
| chr14 | 121037000 | 121038000 | 1956615 | Rap2a | ENSMUSG00000051615 | 0.507 |
| chr14 | 104872000 | 104873000 | 1940450 | Pou4f1 | ENSMUSG00000048349 | 0.035 |
| chr14 | 121905000 | 121906000 | 1957483 | Slc15a1 | ENSMUSG00000025557 | 0.357 |
| chr14 | 57183000 | 57184000 | 1892761 | Rnf17 | ENSMUSG00000000365 | 0.978 |
| chr14 | 106319000 | 106320000 | 1941897 | Spry2 | ENSMUSG00000022114 | 0.123 |
| chr14 | 105999000 | 106000000 | 1941577 | | ENSMUSG00000022116 | 0.981 |
| chr14 | 56719000 | 56720000 | 1892297 | Mcpt8 | ENSMUSG00000022157 | 0.795 |
| chr15 | 8666000 | 8667000 | 1966439 | Slc1a3 | ENSMUSG00000005360 | 0.031 |
| chr15 | 5586000 | 5587000 | 1963359 | Ptger4 | ENSMUSG00000039942 | 0.985 |
| chr15 | 89152000 | 89153000 | 2046871 | Sbf1 | ENSMUSG00000036529 | 0.617 |
| chr15 | 93058000 | 93059000 | 2050777 | Pdzrn4 | ENSMUSG00000036218 | 0.612 |
| chr15 | 12613000 | 12614000 | 1970382 | Pdzd2 | ENSMUSG00000022197 | 0.894 |
| chr15 | 11848000 | 11849000 | 1969617 | Npr3 | ENSMUSG00000022206 | 0.706 |
| chr15 | 92836000 | 92837000 | 2050555 | Pdzrn4 | ENSMUSG00000036218 | 0.591 |
| chr15 | 93229000 | 93230000 | 2050948 | Pphln1 | ENSMUSG00000036167 | 0.077 |
| chr15 | 84494000 | 84495000 | 2042213 | Ldoc1l | ENSMUSG00000055745 | 0.391 |
| chr15 | 64125000 | 64126000 | 2021844 | | ENSMUSG00000078299 | 0.979 |
| chr15 | 10965000 | 10966000 | 1968734 | Slc45a2 | ENSMUSG00000022243 | 0.620 |
| chr15 | 100962000 | 100963000 | 2058681 | Acyrl1 | ENSMUSG00000000530 | 0.567 |
| chr15 | 89231000 | 89232000 | 2046950 | Odf3b | ENSMUSG00000047394 | 0.480 |
| chr15 | 62051000 | 62052000 | 2019770 | H2afy3 | ENSMUSG00000056590 | 0.535 |
| chr15 | 76363000 | 76364000 | 2034082 | Scrt1 | ENSMUSG00000048385 | 0.585 |
| chr15 | 89194000 | 89195000 | 2046913 | Ncaph2 | ENSMUSG00000008690 | 0.975 |
| chr15 | 35232000 | 35233000 | 1993001 | Osr2 | ENSMUSG00000022330 | 0.097 |
| chr15 | 55228000 | 55229000 | 2012947 | Col14a1 | ENSMUSG00000022371 | 0.781 |
| chr15 | 12305000 | 12306000 | 1970074 | Golph3 | ENSMUSG00000022200 | 0.150 |
| chr15 | 103014000 | 103015000 | 2060733 | Smug1 | ENSMUSG00000036061 | 0.147 |
| chr15 | 92920000 | 92921000 | 2050639 | Pdzrn4 | ENSMUSG00000036218 | 0.214 |
| chr15 | 102996000 | 102997000 | 2060715 | Smug1 | ENSMUSG00000036061 | 0.000 |
| chr15 | 76468000 | 76469000 | 2034187 | Vps28 | ENSMUSG00000062381 | 0.970 |
| chr15 | 96238000 | 96239000 | 2053957 | Arid2 | ENSMUSG00000033237 | 0.970 |
| chr15 | 103145000 | 103146000 | 2060864 | Gpr84 | ENSMUSG00000063234 | 0.578 |
| chr15 | 81531000 | 81532000 | 2039250 | Chadl | ENSMUSG00000063765 | 0.794 |
| chr15 | 80282000 | 80283000 | 2038001 | Cacna1i | ENSMUSG00000022416 | 0.502 |
| chr15 | 100304000 | 100305000 | 2058023 | Letmd1 | ENSMUSG00000037353 | 0.969 |
| chr15 | 60989000 | 60990000 | 2018708 | A1bg | ENSMUSG00000022347 | 0.574 |
| chr15 | 62397000 | 62398000 | 2020116 | H2afy3 | ENSMUSG00000056590 | 0.500 |
| chr15 | 86070000 | 86071000 | 2043789 | Tbc1d22a | ENSMUSG00000051864 | 0.610 |
| chr15 | 35317000 | 35318000 | 1993086 | Vps136 | ENSMUSG00000037646 | 0.972 |
| chr15 | 84189000 | 84190000 | 2041908 | Parvg | ENSMUSG00000022439 | 0.340 |
| chr15 | 98957000 | 98958000 | 2056676 | Spats2 | ENSMUSG00000051934 | 0.036 |
| chr15 | 96201000 | 96202000 | 2053920 | Arid2 | ENSMUSG00000033237 | 0.972 |
| chr16 | 72990000 | 72991000 | 2131115 | Robo1 | ENSMUSG00000022883 | 0.970 |
| chr16 | 46495000 | 46496000 | 2104660 | Pvrl3 | ENSMUSG00000022656 | 0.069 |
| chr16 | 44680000 | 44681000 | 2102845 | Boc | ENSMUSG00000022687 | 0.646 |
| chr16 | 69797000 | 69798000 | 2127922 | Cadm2 | ENSMUSG00000064115 | 0.580 |
| chr16 | 70668000 | 70669000 | 2128793 | | ENSMUSG00000062087 | 0.894 |
| chr16 | 44795000 | 44796000 | 2102960 | Cd200r1 | ENSMUSG00000022667 | 0.569 |
| chr16 | 37957000 | 37958000 | 2096122 | Gpr156 | ENSMUSG00000046961 | 0.657 |
| chr16 | 70376000 | 70377000 | 2128501 | Gbe1 | ENSMUSG00000022707 | 0.973 |
| chr16 | 35185000 | 35186000 | 2093350 | Adcy5 | ENSMUSG00000022840 | 0.969 |
| chr16 | 69612000 | 69613000 | 2127737 | Cadm2 | ENSMUSG00000064115 | 0.980 |
| chr16 | 48993000 | 48994000 | 2107158 | Dzip3 | ENSMUSG00000064061 | 0.037 |
| chr16 | 28517000 | 28518000 | 2086682 | Fgf12 | ENSMUSG00000022523 | 0.557 |
| chr16 | 94552000 | 94553000 | 2152456 | Ripply3 | ENSMUSG00000022941 | 0.980 |
| chr16 | 88506000 | 88507000 | 2146631 | Grik1 | ENSMUSG00000022935 | 0.970 |
| chr16 | 37078000 | 37079000 | 2095243 | Polq | ENSMUSG00000034206 | 0.971 |
| chr16 | 38432000 | 38433000 | 2096597 | Popdc2 | ENSMUSG00000022803 | 0.650 |
| chr16 | 44632000 | 44633000 | 2102797 | Boc | ENSMUSG00000022687 | 0.060 |
| chr16 | 37684000 | 37685000 | 2095849 | Ndufb4 | ENSMUSG00000022820 | 0.185 |
| chr16 | 93116000 | 93117000 | 2151241 | Runx1 | ENSMUSG00000022952 | 0.971 |
| chr16 | 77115000 | 77116000 | 2135240 | Usp25 | ENSMUSG00000022867 | 0.980 |
| chr16 | 36199000 | 36200000 | 2094364 | Gm5483 | ENSMUSG00000079597 | 0.390 |
| chr16 | 35230000 | 35231000 | 2093395 | Adcy5 | ENSMUSG00000022840 | 0.574 |
| chr16 | 65629000 | 65630000 | 2123754 | Chmp2b | ENSMUSG00000004843 | 0.516 |
| chr16 | 95680000 | 95681000 | 2153584 | Erg | ENSMUSG00000040732 | 0.000 |
| chr16 | 44099000 | 44100000 | 2102264 | Gramd1c | ENSMUSG00000036292 | 0.969 |
| chr16 | 91321000 | 91322000 | 2149446 | Olig1 | ENSMUSG00000046160 | 0.780 |
| chr16 | 94342000 | 94343000 | 2152246 | Sim2 | ENSMUSG00000062713 | 0.642 |
| chr16 | 96621000 | 96622000 | 2154525 | Pcp4 | ENSMUSG00000000159 | 0.608 |
| chr16 | 87843000 | 87844000 | 2145968 | | ENSMUSG00000055972 | 0.393 |
| chr16 | 91248000 | 91249000 | 2149373 | Olig2 | ENSMUSG00000039830 | 0.656 |
| chr16 | 44308000 | 44309000 | 2102473 | Gm608 | ENSMUSG00000068284 | 0.482 |
| chr16 | 35156000 | 35157000 | 2093321 | Adcy5 | ENSMUSG00000022840 | 0.043 |
| chr16 | 95822000 | 95823000 | 2153726 | Erg | ENSMUSG00000040732 | 0.655 |
| chr16 | 77077000 | 77078000 | 2135202 | Usp25 | ENSMUSG00000022867 | 0.972 |
| chr16 | 48449000 | 48450000 | 2106614 | Morc1 | ENSMUSG00000022652 | 0.970 |
| chr17 | 87535000 | 87536000 | 2240396 | Socs5 | ENSMUSG00000037104 | 0.982 |
| chr17 | 14106000 | 14107000 | 2167126 | Gm7168 | ENSMUSG00000067941 | 0.894 |
| chr17 | 73266000 | 73267000 | 2226176 | Ypel5 | ENSMUSG00000039770 | 0.001 |
| chr17 | 25014000 | 25015000 | 2178031 | Hagh | ENSMUSG00000024158 | 0.589 |
| chr17 | 49153000 | 49154000 | 2202162 | Lrfn2 | ENSMUSG00000040490 | 0.654 |
| chr17 | 24950000 | 24951000 | 2177967 | Hs3st6 | ENSMUSG00000039628 | 0.524 |
| chr17 | 64898000 | 64899000 | 2217808 | Pja2 | ENSMUSG00000024083 | 0.519 |
| chr17 | 27336000 | 27337000 | 2180353 | Ip6k3 | ENSMUSG00000024210 | 0.522 |
| chr17 | 56616000 | 56617000 | 2209625 | Ptprs | ENSMUSG00000013236 | 0.588 |
| chr17 | 87778000 | 87779000 | 2240639 | Ttc7 | ENSMUSG00000036918 | 0.621 |
| chr17 | 8201000 | 8202000 | 2161321 | Rsph3a | ENSMUSG00000073471 | 0.658 |
| chr17 | 29571000 | 29572000 | 2182588 | Fgd2 | ENSMUSG00000024013 | 0.985 |
| chr17 | 71600000 | 71601000 | 2224510 | Lpin2 | ENSMUSG00000024052 | 0.215 |
| chr17 | 25366000 | 25367000 | 2178383 | Unkl | ENSMUSG00000015127 | 0.655 |
| chr17 | 40678000 | 40679000 | 2193695 | Crisp1 | ENSMUSG00000025431 | 0.781 |
| chr17 | 76215000 | 76216000 | 2229076 | Fam98a | ENSMUSG00000002017 | 0.595 |
| chr17 | 32967000 | 32968000 | 2185984 | Zfp799 | ENSMUSG00000059000 | 0.000 |
| chr17 | 86656000 | 86657000 | 2239517 | Prkce | ENSMUSG00000045038 | 0.660 |
| chr17 | 68263000 | 68264000 | 2221173 | Lama1 | ENSMUSG00000032796 | 0.587 |
| chr17 | 32541000 | 32542000 | 2185558 | Rasal3 | ENSMUSG00000052142 | 0.968 |
| chr17 | 86148000 | 86149000 | 2239009 | Six2 | ENSMUSG00000024134 | 0.645 |
| chr17 | 86663000 | 86664000 | 2239524 | Prkce | ENSMUSG00000045038 | 0.986 |
| chr17 | 27338000 | 27339000 | 2180355 | Ip6k3 | ENSMUSG00000024210 | 0.531 |
| chr17 | 86702000 | 86703000 | 2239563 | Prkce | ENSMUSG00000045038 | 0.507 |
| chr17 | 31418000 | 31419000 | 2184435 | Rsph1 | ENSMUSG00000024033 | 0.607 |
| chr17 | 88122000 | 88123000 | 2240983 | Msh2 | ENSMUSG00000024151 | 0.968 |
| chr17 | 69736000 | 69737000 | 2222646 | Zfp161 | ENSMUSG00000049672 | 0.970 |
| chr17 | 86358000 | 86359000 | 2239219 | Six2 | ENSMUSG00000024134 | 0.361 |
| chr17 | 87846000 | 87847000 | 2240707 | Calm2 | ENSMUSG00000036438 | 0.002 |
| chr17 | 29497000 | 29498000 | 2182514 | Fgd2 | ENSMUSG00000024013 | 0.035 |
| chr17 | 28669000 | 28670000 | 2181686 | | ENSMUSG00000024223 | 0.601 |
| chr17 | 8453000 | 8454000 | 2161573 | Ccr6 | ENSMUSG00000040899 | 0.530 |
| chr17 | 15929000 | 15930000 | 2168949 | Chd1 | ENSMUSG00000023852 | 0.893 |
| chr17 | 43106000 | 43107000 | 2196123 | Cd2ap | ENSMUSG00000061665 | 0.659 |
| chr17 | 6988000 | 6989000 | 2160163 | Ezr | ENSMUSG00000052397 | 0.510 |
| chr18 | 6345000 | 6346000 | 2251479 | | ENSMUSG00000073640 | 0.981 |
| chr18 | 64653000 | 64654000 | 2309787 | Fech | ENSMUSG00000024588 | 0.592 |
| chr18 | 7719000 | 7720000 | 2252853 | Mpp7 | ENSMUSG00000057440 | 0.493 |
| chr18 | 82658000 | 82659000 | 2327623 | Mbp | ENSMUSG00000041607 | 0.608 |
| chr18 | 56728000 | 56729000 | 2301862 | Aldh7a1 | ENSMUSG00000053644 | 0.184 |
| chr18 | 57189000 | 57190000 | 2302323 | | ENSMUSG00000024592 | 0.498 |
| chr18 | 66564000 | 66565000 | 2311698 | Ccbe1 | ENSMUSG00000046318 | 0.132 |
| chr18 | 81827000 | 81828000 | 2326824 | Sall3 | ENSMUSG00000024565 | 0.989 |
| chr18 | 24166000 | 24167000 | 2269300 | Zfp35 | ENSMUSG00000063281 | 0.992 |
| chr18 | 37646000 | 37647000 | 2282780 | Pcdhb17 | ENSMUSG00000046387 | 0.620 |
| chr18 | 53553000 | 53554000 | 2298687 | Snx24 | ENSMUSG00000024535 | 0.968 |
| chr18 | 67296000 | 67297000 | 2312430 | Gnal | ENSMUSG00000024524 | 0.657 |
| chr18 | 39029000 | 39030000 | 2284163 | Fgf1 | ENSMUSG00000036585 | 0.781 |
| chr18 | 11424000 | 11425000 | 2256558 | Gata6 | ENSMUSG00000005836 | 0.795 |
| chr18 | 46970000 | 46971000 | 2292104 | Ap3s1 | ENSMUSG00000024480 | 0.969 |
| chr18 | 62149000 | 62150000 | 2307283 | Sh3tc2 | ENSMUSG00000045629 | 0.980 |
| chr18 | 56754000 | 56755000 | 2301888 | | ENSMUSG00000032900 | 0.642 |
| chr18 | 78134000 | 78135000 | 2323131 | Pstpip2 | ENSMUSG00000025429 | 0.178 |
| chr18 | 36124000 | 36125000 | 2281258 | Psd2 | ENSMUSG00000024347 | 0.035 |
| chr18 | 9472000 | 9473000 | 2254606 | Ccny | ENSMUSG00000024286 | 0.972 |
| chr18 | 11169000 | 11170000 | 2256303 | Gata6 | ENSMUSG00000005836 | 0.558 |
| chr18 | 77108000 | 77109000 | 2322112 | Smad2 | ENSMUSG00000024563 | 0.660 |
| chr18 | 56618000 | 56619000 | 2301752 | Gramd3 | ENSMUSG00000001700 | 0.384 |
| chr18 | 66627000 | 66628000 | 2311761 | Pmaip1 | ENSMUSG00000024521 | 0.603 |
| chr18 | 12706000 | 12707000 | 2257840 | Lama3 | ENSMUSG00000024421 | 0.887 |
| chr18 | 11905000 | 11906000 | 2257039 | Rbbp8 | ENSMUSG00000041238 | 0.969 |
| chr18 | 67438000 | 67439000 | 2312572 | Mppe1 | ENSMUSG00000062526 | 0.202 |
| chr18 | 10324000 | 10325000 | 2255458 | Rock1 | ENSMUSG00000024290 | 0.487 |
| chr18 | 70663000 | 70664000 | 2315797 | Stard6 | ENSMUSG00000079608 | 0.582 |
| chr18 | 13223000 | 13224000 | 2258357 | Hrh4 | ENSMUSG00000037346 | 0.490 |
| chr18 | 80559000 | 80560000 | 2325556 | Kcng2 | ENSMUSG00000059852 | 0.036 |
| chr18 | 57380000 | 57381000 | 2302514 | Megf10 | ENSMUSG00000024593 | 0.978 |
| chr18 | 37424000 | 37425000 | 2282558 | Pcdhb1 | ENSMUSG00000051663 | 0.132 |
| chr18 | 12631000 | 12632000 | 2257765 | Lama3 | ENSMUSG00000024421 | 0.664 |
| chr18 | 61534000 | 61535000 | 2306668 | | ENSMUSG00000069367 | 0.992 |
| chr19 | 32517000 | 32518000 | 2365055 | Sgms1 | ENSMUSG00000040451 | 0.978 |
| chr19 | 19316000 | 19317000 | 2351854 | Rorb | ENSMUSG00000036192 | 0.780 |
| chr19 | 28813000 | 28814000 | 2361351 | Glis3 | ENSMUSG00000052942 | 0.780 |
| chr19 | 26228000 | 26229000 | 2358766 | Dmrt2 | ENSMUSG00000048138 | 0.609 |
| chr19 | 53632000 | 53633000 | 2386170 | Dusp5 | ENSMUSG00000034765 | 0.255 |
| chr19 | 53728000 | 53729000 | 2386266 | Smc3 | ENSMUSG00000024974 | 0.593 |
| chr19 | 53403000 | 53404000 | 2385941 | Mxi1 | ENSMUSG00000025025 | 0.001 |
| chr19 | 30525000 | 30526000 | 2363063 | Mbl2 | ENSMUSG00000024863 | 0.659 |
| chr19 | 47520000 | 47521000 | 2380058 | Gm5098 | ENSMUSG00000078104 | 0.551 |
| chr19 | 53067000 | 53068000 | 2385605 | Ins1 | ENSMUSG00000035804 | 0.522 |
| chr19 | 53914000 | 53915000 | 2386452 | Rbm20 | ENSMUSG00000043639 | 0.974 |
| chr19 | 18952000 | 18953000 | 2351490 | Trpm6 | ENSMUSG00000024727 | 0.642 |
| chr19 | 8912000 | 89130002 | 341450 | Hnmpul2 | ENSMUSG00000071659 | 0.061 |
| chr19 | 45107000 | 45108000 | 2377645 | Pdzd7 | ENSMUSG00000074818 | 0.652 |
| chr19 | 41372000 | 41373000 | 2373910 | Tm9sf3 | ENSMUSG00000025016 | 1.000 |
| chr19 | 25488000 | 25489000 | 2358026 | Kank1 | ENSMUSG00000032702 | 0.571 |
| chr19 | 58750000 | 58751000 | 2391288 | Pnlip | ENSMUSG00000046008 | 0.974 |
| chr19 | 53756000 | 53757000 | 2386294 | Rbm20 | ENSMUSG00000043639 | 0.339 |
| chr19 | 46835000 | 46836000 | 2379373 | As3mt | ENSMUSG00000003559 | 0.000 |
| chr19 | 55585000 | 55586000 | 2388123 | Vti1a | ENSMUSG00000024983 | 0.974 |
| chr19 | 17507000 | 17508000 | 2350045 | Rfk | ENSMUSG00000024712 | 0.978 |
| chr19 | 10366000 | 10367000 | 2342904 | Gm98 | ENSMUSG00000036098 | 0.482 |
| chr19 | 55149000 | 55150000 | 2387687 | Adra2a | ENSMUSG00000033717 | 0.970 |
| chr19 | 47857000 | 47858000 | 2380395 | | ENSMUSG00000044948 | 0.593 |
| chr19 | 36132000 | 36133000 | 2368670 | Htr7 | ENSMUSG00000024798 | 0.130 |
| chr19 | 41675000 | 41676000 | 2374213 | AI606181 | ENSMUSG00000074873 | 0.570 |
| chr19 | 30565000 | 30566000 | 2363103 | Mbl2 | ENSMUSG00000024863 | 0.211 |
| chr19 | 9018000 | 9019000 | 2341556 | | ENSMUSG00000072030 | 0.089 |
| chr19 | 45891000 | 45892000 | 2378429 | Kcnip2 | ENSMUSG00000025221 | 0.565 |
| chr19 | 16673000 | 16674000 | 2349211 | Gna14 | ENSMUSG00000024697 | 0.403 |
| chr19 | 53895000 | 53896000 | 2386433 | Rbm20 | ENSMUSG00000043639 | 0.617 |
| chr19 | 46545000 | 46546000 | 2379083 | Sufu | ENSMUSG00000025231 | 0.664 |
| chr19 | 37765000 | 37766000 | 2370303 | Cyp26c1 | ENSMUSG00000062432 | 0.535 |
| chr19 | 46399000 | 46400000 | 2378937 | Psd | ENSMUSG00000037126 | 0.600 |
| chr19 | 33836000 | 33837000 | 2366374 | AI747699 | ENSMUSG00000024766 | 0.077 |
| chrX | 49967000 | 49968000 | 2439805 | Gpc3 | ENSMUSG00000055653 | 0.030 |
| chrX | 78812000 | 78813000 | 2468491 | | ENSMUSG00000060673 | 0.590 |
| chrX | 6577000 | 6578000 | 2397159 | Dgkk | ENSMUSG00000062393 | 0.893 |
| chrX | 35994000 | 35995000 | 2425832 | C1galt1c1 | ENSMUSG00000048970 | 0.584 |
| chrX | 87250000 | 87251000 | 2476929 | | ENSMUSG00000035387 | 0.660 |
| chrX | 72445000 | 72446000 | 2462124 | | ENSMUSG00000073094 | 0.893 |
| chrX | 96789000 | 96790000 | 2486468 | Pja1 | ENSMUSG00000034403 | 0.104 |
| chrX | 73119000 | 73120000 | 2462798 | Pls3 | ENSMUSG00000016382 | 0.160 |
| chrX | 46065000 | 46066000 | 2435903 | Rbmx2 | ENSMUSG00000031107 | 0.971 |
| chrX | 83469000 | 83470000 | 2473148 | Nr0b1 | ENSMUSG00000025056 | 0.920 |
| chrX | 153966000 | 153967000 | 2543397 | Sms | ENSMUSG00000071708 | 0.617 |
| chrX | 7721000 | 7722000 | 2398303 | Wdr13 | ENSMUSG00000031166 | 0.420 |
| chrX | 45948000 | 45949000 | 2435786 | Zfp280c | ENSMUSG00000036916 | 0.571 |
| chrX | 71527000 | 71528000 | 2461206 | Dnase111 | ENSMUSG00000019088 | 0.000 |
| chrX | 50266000 | 50267000 | 2440104 | Phf6 | ENSMUSG00000025626 | 0.000 |
| chrX | 35838000 | 35839000 | 2425676 | Lamp2 | ENSMUSG00000016534 | 0.561 |
| chrX | 159421000 | 159422000 | 2548852 | Ctps2 | ENSMUSG00000031360 | 0.972 |
| chrX | 35953000 | 35954000 | 2425791 | Mcts1 | ENSMUSG00000000355 | 0.001 |
| chrX | 39260000 | 39261000 | 2429098 | | ENSMUSG00000081918 | 0.980 |
| chrX | 7650000 | 7651000 | 2398232 | | ENSMUSG00000082572 | 0.000 |
| chrX | 37253000 | 37254000 | 2427091 | Cypt14 | ENSMUSG00000079618 | 0.780 |
| chrX | 49033000 | 49034000 | 2438871 | | ENSMUSG00000082968 | 0.031 |
| chrX | 11069000 | 11070000 | 2401651 | Gm4906 | ENSMUSG00000069038 | 0.185 |
| chrX | 48194000 | 48195000 | 2438032 | | ENSMUSG00000031112 | 0.002 |
| chrX | 54306000 | 54307000 | 2443994 | Htatsf1 | ENSMUSG00000067873 | 0.002 |
| chrX | 7459000 | 7460000 | 2398041 | Pim2 | ENSMUSG00000031155 | 0.972 |
| chrX | 68810000 | 68811000 | 2458489 | Hmgb3 | ENSMUSG00000015217 | 0.043 |
| chrX | 6356000 | 6357000 | 2396988 | Dgkk | ENSMUSG00000062393 | 0.043 |
| chrX | 136406000 | 136407000 | 2525887 | Morc4 | ENSMUSG00000031434 | 0.037 |
| chrX | 133634000 | 133635000 | 2523115 | | ENSMUSG00000080718 | 0.083 |
| chrX | 12410000 | 12411000 | 2402992 | Med14 | ENSMUSG00000064127 | 0.344 |
| chrX | 91367000 | 91368000 | 2481046 | | ENSMUSG00000081055 | 0.117 |
| chrX | 97016000 | 97017000 | 2486695 | Tmem28 | ENSMUSG00000071719 | 0.069 |
| chrX | 46847000 | 46848000 | 2436685 | | ENSMUSG00000036198 | 0.069 |
| chrX | 39421000 | 39422000 | 2429259 | Xiap | ENSMUSG00000025860 | 0.031 |
| chrY | 293000 | 294000 | 2556276 | Kdm5d | ENSMUSG00000056673 | 0.826 |
| chrY | 325000 | 326000 | 2556308 | Kdm5d | ENSMUSG00000056673 | 0.784 |
| chrY | 334000 | 335000 | 2556317 | | ENSMUSG00000075874 | 0.851 |
| chrY | 335000 | 336000 | 2556318 | | ENSMUSG00000075874 | 0.778 |
| chrY | 456000 | 457000 | 2556439 | Eif2s3y | ENSMUSG00000069049 | 0.818 |
| chrY | 699000 | 700000 | 2556682 | | ENSMUSG00000077793 | 0.959 |
| chrY | 817000 | 818000 | 2556800 | Usp9y | ENSMUSG00000069044 | 0.767 |
| chrY | 818000 | 819000 | 2556801 | Usp9y | ENSMUSG00000069044 | 0.878 |
| chrY | 917000 | 918000 | 2556900 | Usp9y | ENSMUSG00000069044 | 0.626 |
| chrY | 936000 | 937000 | 2556919 | Usp9y | ENSMUSG00000069044 | 0.940 |
| chrY | 948000 | 949000 | 2556931 | Usp9y | ENSMUSG00000069044 | 0.820 |
| chrY | 956000 | 957000 | 2556939 | Usp9y | ENSMUSG00000069044 | 0.870 |
| chrY | 961000 | 962000 | 2556944 | Usp9y | ENSMUSG00000069044 | 0.859 |
| chrY | 1109000 | 1110000 | 2557092 | Usp9y | ENSMUSG00000069044 | 0.870 |
| chrY | 1126000 | 1127000 | 2557109 | Usp9y | ENSMUSG00000069044 | 0.915 |
| chrY | 1146000 | 1147000 | 2557129 | Usp9y | ENSMUSG00000069044 | 0.925 |
| chrY | 1156000 | 1157000 | 2557139 | Usp9y | ENSMUSG00000069044 | 0.725 |
| chrY | 1310000 | 1311000 | 2557293 | Usp9y | ENSMUSG00000069044 | 0.910 |
| chrY | 1420000 | 1421000 | 2557403 | Usp9y | ENSMUSG00000069044 | 0.910 |
| chrY | 1454000 | 1455000 | 2557437 | Zfy2 | ENSMUSG00000000103 | 0.945 |
| chrY | 1460000 | 1461000 | 2557443 | Zfy2 | ENSMUSG00000000103 | 0.785 |
| chrY | 1464000 | 1465000 | 2557447 | Zfy2 | ENSMUSG00000000103 | 0.865 |
| chrY | 1537000 | 1538000 | 2557520 | Zfy2 | ENSMUSG00000000103 | 0.850 |
| chrY | 1617000 | 1618000 | 2557600 | Zfy2 | ENSMUSG00000000103 | 0.905 |
| chrY | 1618000 | 1619000 | 2557601 | Zfy2 | ENSMUSG00000000103 | 0.870 |
| chrY | 1664000 | 1665000 | 2557647 | Zfy2 | ENSMUSG00000000103 | 0.830 |
| chrY | 1779000 | 1780000 | 2557762 | Zfy2 | ENSMUSG00000000103 | 0.865 |
| chrY | 1801000 | 1802000 | 2557784 | Zfy2 | ENSMUSG00000000103 | 0.945 |
| chrY | 1839000 | 1840000 | 2557822 | Zfy2 | ENSMUSG00000000103 | 0.900 |
| chrY | 1840000 | 1841000 | 2557823 | Zfy2 | ENSMUSG00000000103 | 0.910 |
| chrY | 1858000 | 1859000 | 2557841 | Zfy2 | ENSMUSG00000000103 | 0.920 |
| chrY | 1875000 | 1876000 | 2557858 | Zfy2 | ENSMUSG00000000103 | 0.875 |
| chrY | 1973000 | 1974000 | 2557956 | Sry | ENSMUSG00000069036 | 0.915 |
| chrY | 2016000 | 2017000 | 2557999 | Sry | ENSMUSG00000069036 | 0.835 |
| chrY | 2035000 | 2036000 | 2558018 | Sry | ENSMUSG00000069036 | 0.935 |

Induced hematopoietic stem cells are made by the hand of man by, e.g., modifying the gene expression of at least one of the factors disclosed herein of a somatic cell, a pluripotent cell, a progenitor cell or a stem cell, or by exposing any one of these cell types to at least one protein or RNA that produces at least one protein as disclosed herein. The cells can further be made by exposing them to small molecules that turn on at least one of the factors disclosed herein. In some aspects at least two, three, four, five, six, seven, or eight factors are used to make the induced hematopoietic stem cells.

The induced hematopoietic stem cells as described herein differ from naturally occurring hematopoietic stem cells by both their posttranslational modification signatures and their gene expression signatures. These differences are passed along to their progeny. Therefore, also their progeny, whether clonal or differentiated, differs from the naturally occurring differentiated cells.

Induced hematopoietic stem cell as it is defined in some aspects of all the embodiments of the invention comprise, consist essentially of or consist of cells that are functionally capable of copying themselves as well as differentiating into various cells of hematopoietic lineage. In other words, they can be defined as having multilineage potential.

Induced hematopoietic stem cell is also defined as comprising a gene expression signature that differs from naturally occurring hematopoietic stem cells. One can experimentally show the difference by comparing the gene expression pattern of a naturally occurring hematopoietic stem cell to that of the induced hematopoietic stem cells. For example, the gene expression signature can differ in regard to the genes as shown in Tables 2 or 3. Therefore, in some aspects of all the embodiments of the invention, the induced hematopoietic stem cells comprise an expression signature that is about 1-5%, 5-10%, 5-15%, or 5-20% different from the expression signature of about 1-5%, 2-5%, 3-5%, up to 50%, up to 40%, up to 30%, up to 25%, up to 20%, up to 15%, or up to 10% of the genes of Tables 2 or 3.

Induced hematopoietic stem cell is further defined as comprising a posttranslational modification signature that differs from naturally occurring hematopoietic stem cells. In some embodiments, the posttranslational modification is methylation. For example, the methylation pattern of the induced hematopoietic stem cells is in some aspects about 1-5%, in some aspects 1-10%, in some aspects 5-10% different from the methylation pattern at about 1-5%, 1-10%, 5-10%, up to 50%, up to 40%, up to 30%, up to 25%, up to 20%, up to 15%, or up to 10% of the methylation sites shown in Table 4. In some aspects, the amount of methylation in the iHSC differs from the isolated or endogenous HSCs by no more than 1%, 2%, 3%, 4% or no more than 5%, for example as compared to the amount of methylation in the example loci listed in Table 4. Other methylation sites can naturally be used as well in any comparison for differentiating the iHSCs from HSCs.

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

As used herein and in the claims, the singular forms include the plural reference and vice versa unless the context clearly indicates otherwise. The term “or” is inclusive unless modified, for example, by “either.” Other than in the operating examples, or where otherwise indicated, all numbers expressing quantities of ingredients or reaction conditions used herein should be understood as modified in all instances by the term “about.”

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

Unless defined otherwise, all technical and scientific terms used herein have the same meaning as those commonly understood to one of ordinary skill in the art to which this invention pertains. Although any known methods, devices, and materials may be used in the practice or testing of the invention, the methods, devices, and materials in this regard are described herein.

Some embodiments of the invention are listed in the following paragraphs:
1. A hematopoietic stem cell (HSC) inducing composition comprising one or more expression vectors encoding at least one, two, three, four, five, six, seven, eight, or more HSC inducing factors selected from: CDKN1C, DNMT3B, EGR1, ETV6, EVI1, GATA2, GFI1B, GLIS2, HLF, HMGA2, HOXA5, HOXA9, HOXB3, HOXB4, HOXB5, IGF2BP2, IKZF2, KLF12, KLF4, KLF9, LMO2, MEIS1, MSI2, MYCN, NAP1L3, NDN, NFIX, NKX2-3, NR3C2, PBX1, PRDM16, PRDM5, RARB, RBBP6, RBPMS, RUNX1, RUNX1T1, SMAD6, TAL1, TCF15, VDR, ZFP37, ZFP467, ZFP521, ZFP532, and ZFP612.
2. The HSC inducing composition of paragraph 1, wherein the at least one, two, three, four, or more HSC inducing factors are HLF, RUNX1T1, PBX1, LMO2, PRDM5, ZFP37, MYCN, MSI2, NKX2-3, MEIS1, and RBPMS.
3. The HSC inducing composition of paragraph 1, wherein the at least one, two, three, four, or more HSC inducing factors are HLF, RUNX1T1, ZFP37, PBX1, LMO2, and PRDM5.
4. A hematopoietic stem cell (HSC) inducing composition comprising one or more expression vectors comprising:
   a. a nucleic acid sequence encoding HLF;
   b. a nucleic acid sequence encoding RUNX1T1;
   c. a nucleic acid sequence encoding ZFP37;
   d. a nucleic acid sequence encoding PBX1;
   e. a nucleic acid sequence encoding LMO2; and
   f. a nucleic acid sequence encoding PRDM5.
5. The HSC inducing composition of paragraph 4, further comprising one or more of:
   a. a nucleic acid sequence encoding PRDM16;
   b. a nucleic acid sequence encoding ZFP467; and
   c. a nucleic acid sequence encoding VDR.
6. A hematopoietic stem cell (HSC) inducing composition comprising one or more expression vectors comprising:
   a. a nucleic acid sequence encoding HLF;
   b. a nucleic acid sequence encoding RUNX1T1;
   c. a nucleic acid sequence encoding PBX1;
   d. a nucleic acid sequence encoding LMO2;
   e. a nucleic acid sequence encoding PRDM5
   f. a nucleic acid sequence encoding ZFP37;
   g. a nucleic acid sequence encoding MYCN;
   h. a nucleic acid sequence encoding MSI2;
   i. a nucleic acid sequence encoding NKX2-3;
   j. a nucleic acid sequence encoding MEIS1; and
   k. a nucleic acid sequence encoding RBPMS.
7. A hematopoietic stem cell (HSC) inducing composition comprising one or more expression vectors comprising:
   a. a nucleic acid sequence encoding ZFP467;
   b. a nucleic acid sequence encoding PBX1;
   c. a nucleic acid sequence encoding HOXB4; and
   d. a nucleic acid sequence encoding MSI2.
8. The HSC inducing composition of paragraph 7, further comprising one or more of:
   a. a nucleic acid sequence encoding HLF;
   b. a nucleic acid sequence encoding LMO2;
   c. a nucleic acid sequence encoding PRDM16; and
   d. a nucleic acid sequence encoding ZFP37.
9. A hematopoietic stem cell (HSC) inducing composition comprising one or more expression vectors comprising:
   a. a nucleic acid sequence encoding MYCN;
   b. a nucleic acid sequence encoding MSI2;
   c. a nucleic acid sequence encoding NKX2-3; and
   d. a nucleic acid sequence encoding RUNX1T1.
10. The HSC inducing composition of paragraph 9, further comprising one or more of:
   a. a nucleic acid sequence encoding HOXB5;
   b. a nucleic acid sequence encoding HLF;
   c. a nucleic acid sequence encoding ZFP467;
   d. a nucleic acid sequence encoding HOXB3;
   e. a nucleic acid sequence encoding LMO2;
   f. a nucleic acid sequence encoding PBX1;
   g. a nucleic acid sequence encoding ZFP37; and
   h. a nucleic acid sequence encoding ZFP521.
11. A hematopoietic stem cell (HSC) inducing composition comprising one or more expression vectors composition comprising:
   a. a nucleic acid sequence encoding HOXB4;
   b. a nucleic acid sequence encoding PBX1;
   c. a nucleic acid sequence encoding LMO2;
   d. a nucleic acid sequence encoding ZFP467; and
   e. a nucleic acid sequence encoding ZFP521.
12. The HSC inducing composition of paragraph 11, further comprising one or more of:
   a. a nucleic acid sequence encoding KLF12;
   b. a nucleic acid sequence encoding HLF; and
   c. a nucleic acid sequence encoding EGR1.
13. A hematopoietic stem cell (HSC) inducing composition comprising one or more expression vectors comprising:
   a. a nucleic acid sequence encoding MEIS1;
   b. a nucleic acid sequence encoding RBPMS;
   c. a nucleic acid sequence encoding ZFP37;
   d. a nucleic acid sequence encoding RUNX1T1; and
   e. a nucleic acid sequence encoding LMO2.
14. The HSC inducing composition of paragraph 13, further comprising one or more of:
   a. a sequence encoding KLF12; and
   b. a sequence encoding HLF;
15. A hematopoietic stem cell (HSC) inducing composition comprising one or more expression vectors comprising:
   a. a nucleic acid sequence encoding ZFP37;
   b. a nucleic acid sequence encoding HOXB4;
   c. a nucleic acid sequence encoding LMO2; and
   d. a nucleic acid sequence encoding HLF.
16. The HSC inducing composition of paragraph 15, further comprising one or more of:
   a. a nucleic acid sequence encoding MYCN;
   b. a nucleic acid sequence encoding ZFP467;
   c. a nucleic acid sequence encoding NKX2-3
   d. a nucleic acid sequence encoding PBX1; and
   e. a nucleic acid sequence encoding KLF4.
17. The HSC inducing compositions of any one of paragraphs 4-16, wherein the one or more expression vectors are retroviral vectors.
18. The HSC inducing compositions of any one of paragraphs 4-16, wherein the one or more expression vectors are lentiviral vectors.

19. The HSC inducing composition of paragraph 18, wherein the lentiviral vectors are inducible lentiviral vectors.
20. A method for preparing an induced hematopoietic stem cell (iHSC) from a somatic cell comprising:
   a. transducing the somatic cell with one or more vectors comprising a nucleic acid sequence encoding HLF, a nucleic acid sequence encoding RUNX1T1; a nucleic acid sequence encoding ZFP37; a nucleic acid sequence encoding PBX1; a nucleic acid sequence encoding LMO2; and a nucleic acid sequence encoding PRDM5, wherein each said nucleic acid sequence is operably linked to a promoter; and
   b. culturing the transduced somatic cell in a cell media that supports growth of hematopoietic stem cells, thereby preparing an iHSC.
21. The method of paragraph 20, wherein the transducing of step (a) further comprises one or more vectors comprising one or more of: a nucleic acid sequence encoding PRDM16 a nucleic acid sequence encoding ZFP467; and a nucleic acid sequence encoding VDR.
22. A method for preparing an induced hematopoietic stem cell (iHSC) from a somatic cell comprising:
   a. transducing the somatic cell with one or more vectors comprising a nucleic acid sequence encoding HLF, a nucleic acid sequence encoding RUNX1T1; a nucleic acid sequence encoding PBX1; a nucleic acid sequence encoding LMO2; a nucleic acid sequence encoding PRDM5; a nucleic acid sequence encoding ZFP37; a nucleic acid sequence encoding MYCN; a nucleic acid sequence encoding MSI2; a nucleic acid sequence encoding NKX2-3; a nucleic acid sequence encoding MEIS1; and a nucleic acid sequence encoding RBPMS; wherein each said nucleic acid sequence is operably linked to a promoter; and
   b. culturing the transduced somatic cell in a cell media that supports growth of hematopoietic stem cells, thereby preparing an iHSC.
23. A method for preparing an induced hematopoietic stem cell (iHSC) from a somatic cell comprising:
   a. transducing the somatic cell with one or more vectors comprising a nucleic acid sequence encoding ZFP467, a nucleic acid sequence encoding PBX1; a nucleic acid sequence encoding HOXB4; and a nucleic acid sequence encoding MSI2; wherein each said nucleic acid sequence is operably linked to a promoter; and
   b. culturing the transduced somatic cell in a cell media that supports growth of hematopoietic stem cells, thereby preparing an iHSC.
24. The method of paragraph 23, wherein the transducing of step (a) further comprises one or more vectors comprising one or more of: a nucleic acid sequence encoding HLF, a nucleic acid sequence encoding LMO2; a nucleic acid sequence encoding PRDM16; and a nucleic acid sequence encoding ZFP37.
25. A method for preparing an induced hematopoietic stem cell (iHSC) from a somatic cell comprising:
   a. transducing the somatic cell with one or more vectors comprising a nucleic acid sequence encoding MYCN; a nucleic acid sequence encoding MSI2, a nucleic acid sequence encoding NKX2-3; and a nucleic acid sequence encoding RUNX1T1; wherein each said nucleic acid sequence is operably linked to a promoter; and
   b. culturing the transduced somatic cell in a cell media that supports growth of hematopoietic stem cells, thereby preparing an iHSC.
26. The method of paragraph 25, wherein the transducing of step (a) further comprises one or more vectors comprising one or more of: a nucleic acid sequence encoding HOXB5; a nucleic acid sequence encoding HLF, a nucleic acid sequence encoding ZFP467; a nucleic acid sequence encoding HOXB3; a nucleic acid sequence encoding LMO2; a nucleic acid sequence encoding PBX1; a nucleic acid sequence encoding ZFP37; and a nucleic acid sequence encoding ZFP521.
27. A method for preparing an induced hematopoietic stem cell (iHSC) from a somatic cell comprising:
   a. transducing the somatic cell with one or more vectors comprising a nucleic acid sequence encoding HOXB4; a nucleic acid sequence encoding PBX1, a nucleic acid sequence encoding LMO2; a nucleic acid sequence encoding ZFP467; and a nucleic acid sequence encoding ZFP521; wherein each said nucleic acid sequence is operably linked to a promoter; and
   b. culturing the transduced somatic cell in a cell media that supports growth of hematopoietic stem cells, thereby preparing an iHSC.
28. The method of paragraph 27, wherein the transducing of step (a) further comprises one or more vectors comprising one or more of: a nucleic acid sequence encoding KLF12; a nucleic acid sequence encoding HLF; and a nucleic acid sequence encoding EGR1.
29. A method for preparing an induced hematopoietic stem cell (iHSC) from a somatic cell comprising:
   a. transducing the somatic cell with one or more vectors comprising a nucleic acid sequence encoding MEIS1; a nucleic acid sequence encoding RBPMS; a nucleic acid sequence encoding ZFP37; a nucleic acid sequence encoding RUNX1T1; and a nucleic acid sequence encoding LMO2; wherein each said nucleic acid sequence is operably linked to a promoter; and
   b. culturing the transduced somatic cell in a cell media that supports growth of hematopoietic stem cells, thereby preparing an iHSC.
30. The method of paragraph 29, wherein the transducing of step (a) further comprises one or more vectors comprising one or more of: a nucleic acid sequence encoding KLF12; and a nucleic acid sequence encoding HLF.
31. A method for preparing an induced hematopoietic stem cell (iHSC) from a somatic cell comprising:
   a. transducing the somatic cell with one or more vectors comprising a nucleic acid sequence encoding ZFP37; a nucleic acid sequence encoding HOXB4; a nucleic acid sequence encoding LMO2; and a nucleic acid sequence encoding HLF; wherein each said nucleic acid sequence is operably linked to a promoter; and
   b. culturing the transduced somatic cell in a cell media that supports growth of hematopoietic stem cells, thereby preparing an iHSC.
32. The method of paragraph 31, wherein the transducing of step (a) further comprises one or more vectors comprising one or more of: a nucleic acid sequence encoding KLF12; and a nucleic acid sequence encoding HLF.
33. A method for preparing an induced hematopoietic stem cell (iHSC) from a somatic cell comprising:
   a. transducing the somatic cell with one or more vectors comprising a nucleic acid sequence encoding ZFP37; a nucleic acid sequence encoding HOXB4; a nucleic acid sequence encoding LMO2; and a nucleic acid sequence encoding HLF; wherein each said nucleic acid sequence is operably linked to a promoter; and
   b. culturing the transduced somatic cell in a cell media that supports growth of hematopoietic stem cells, thereby preparing an iHSC.
34. The method of paragraph 33, wherein the transducing of step (a) further comprises one or more vectors comprising one or more of: a nucleic acid sequence encoding MYCN; a nucleic acid sequence encoding ZFP467; a nucleic acid sequence encoding NKX2-3; a nucleic acid sequence encoding PBX1; and a nucleic acid sequence encoding KLF4.
35. The method of any one of paragraphs 20-34, wherein the somatic cell is a fibroblast cell.
36. The method of any one of paragraphs 20-34, wherein the somatic cell is a hematopoietic lineage cell.
37. The method of paragraph 36, wherein the hematopoietic lineage cell is selected from promyelocytes, neutrophils, eosinophils, basophils, reticulocytes, erythrocytes, mast cells, osteoclasts, megakaryoblasts, platelet producing megakaryocytes, platelets, monocytes, macrophages, dendritic cells, lymphocytes, NK cells, NKT cells, innate lymphocytes, multipotent hematopoietic progenitor cells, oligopotent hematopoietic progenitor cells, and lineage restricted hematopoietic progenitors.
38. The method of paragraph 36, wherein the hematopoietic lineage cell is selected from a multi-potent progenitor cell (MPP), common myeloid progenitor cell (CMP), granulocyte-monocyte progenitor cells (GMP), common lymphoid progenitor cell (CLP), and pre-megakaryocyte-erythrocyte progenitor cell.
39. The method of paragraph 36, wherein the hematopoietic lineage cell is selected from a megakaryocyte-erythrocyte progenitor cell (MEP), a ProB cell, a PreB cell, a PreProB cell, a ProT cell, a double-negative T cell, a pro-NK cell, a pro-dendritic cell (pro-DC), pre-granulocyte/macrophage cell, a granulocyte/macrophage progenitor (GMP) cell, and a pro-mast cell (ProMC).
40. A method of promoting transdifferentiation of a ProPreB cell to the myeloid lineage comprising:
   a. transducing a ProPreB cell with one or more vectors comprising a nucleic acid sequence encoding ZFP467, a nucleic acid sequence encoding PBX1; a nucleic acid sequence encoding HOXB4; and a nucleic acid sequence encoding MSI2; wherein each said nucleic acid sequence is operably linked to a promoter; and
   b. culturing the transduced ProPreB cell in a cell media that supports growth of myeloid lineage cells, thereby transdifferentiating the ProPreB cell to the myeloid lineage.
41. The method of paragraph 40, wherein the transducing of step (a) further comprises one or more vectors comprising one or more of: a nucleic acid sequence encoding HLF, a nucleic acid sequence encoding LMO2; a nucleic acid sequence encoding PRDM16; and a nucleic acid sequence encoding ZFP37.
42. A method of increasing survival and/or proliferation of ProPreB cells, comprising:
   a. transducing a ProPreB cell with one or more vectors comprising a nucleic acid sequence encoding HOXB4; a nucleic acid sequence encoding PBX1, a nucleic acid sequence encoding LMO2; a nucleic acid sequence encoding ZFP467; and a nucleic acid sequence encoding ZFP521; wherein each said nucleic acid sequence is operably linked to a promoter; and
   b. culturing the transduced ProPreB cell in a cell media that supports growth of ProPreB cells, thereby increasing survival and/or proliferation of ProPreB cells.
43. The method of paragraph 42, wherein the transducing of step (a) further comprises one or more vectors comprising one or more of: a nucleic acid sequence encoding KLF12; a nucleic acid sequence encoding HLF; and a nucleic acid sequence encoding EGR1.
44. An isolated induced hematopoietic stem cell (iHSC) produced by the method of any one of paragraphs 20-39.
45. A cell clone comprising a plurality of the induced hematopoietic stem cells (iHSCs) of paragraph 44.
46. The cell clone of paragraph 45, further comprising a pharmaceutically acceptable carrier.
47. A kit for making induced hematopoietic stem cells (iHSCs) comprising the HSC inducing compositions comprising one or more expression vector components of any one of paragraphs 1-19.
48. An induced pluripotent stem cell.
49. An induced hematopoietic stem cell induced by contacting a somatic cell, a pluripotent cell, a progenitor cell or a stem cell with at least one of the factors selected from the group consisting of nucleic acid encoding a gene encoding CDKN1C, DNMT3B, EGR1, ETV6, EVI1, GATA2, GFI1B, GLIS2, HLF, HMGA2, HOXA5, HOXA9, HOXB3, HOXB4, HOXB5, IGF2BP2, IKZF2, KLF12, KLF4, KLF9, LMO2, MEIS1, MSI2, MYCN, NAP1L3, NDN, NFIX, NKX2-3, NR3C2, PBX1, PRDM16, PRDM5, RARB, RBBP6, RBPMS, RUNX1, RUNX1T1, SMAD6, TAL1, TCF15, VDR, ZFP37, ZFP467, ZFP521, ZFP532, and ZFP612 or a protein encoded by such gene.
50. The induced hematopoietic stem cell of paragraph 49, wherein the at least one factor is selected from the group consisting of HLF, RUNX1T1, PBX1, LMO2, PRDM5, ZFP37, MYCN, MSI2, NKX2-3, MEIS1, and RBPMS.
51. The induced hematopoietic stem cell of paragraph 49, wherein the at least one factor is selected from the group consisting of HLF, RUNX1T1, ZFP37, PBX1, LMO2, and PRDM5.
52. The induced hematopoietic stem cell of any of paragraphs 49-51, wherein the somatic cell, the pluripotent cell, the progenitor cell or the stem cell is contacted with at least two of the factors.
53. The induced hematopoietic stem cell of any of paragraphs 49-51, wherein the somatic cell, the pluripotent cell, the progenitor cell or the stem cell is contacted with at least three of the factors.
54. The induced hematopoietic stem cell of any of paragraphs 49-51, wherein the somatic cell, the pluripotent cell, the progenitor cell or the stem cell is contacted with at least three of the factors.
55. The induced hematopoietic stem cell of any of paragraphs 49-51, wherein the somatic cell, the pluripotent cell, the progenitor cell or the stem cell is contacted with at least four of the factors.
56. The induced hematopoietic stem cell of any of paragraphs 49-51, wherein the somatic cell, the pluripotent cell, the progenitor cell or the stem cell is contacted with at least five of the factors.
57. The induced hematopoietic stem cell of any of paragraphs 49-51, wherein the somatic cell, the pluripotent cell, the progenitor cell or the stem cell is contacted with at least six of the factors.
58. The induced hematopoietic stem cell of any of paragraphs 49-51, wherein the somatic cell, the pluripotent cell, the progenitor cell or the stem cell is contacted with at least seven of the factors.
59. The induced hematopoietic stem cell of any of paragraphs 49-51, wherein the somatic cell, the pluripotent cell, the progenitor cell or the stem cell is contacted with at least eight of the factors.
60. The induced hematopoietic stem cell of any of paragraphs 49-59, comprising at least one vector.
61. The induced hematopoietic stem cell of paragraph 60, wherein the vector is integrated in the genome of the stem cell.
62. The induced hematopoietic stem cell of any of paragraphs 49-61, wherein the somatic cell is a fibroblast cell.
63. The induced hematopoietic stem cell of any of paragraphs 49-61, wherein the somatic cell is a hematopoietic lineage cell.
64. The induced hematopoietic stem cell of paragraph 63, wherein the hematopoietic lineage cell is selected from promyelocytes, neutrophils, eosinophils, basophils, reticulocytes, erythrocytes, mast cells, osteoclasts, megakaryoblasts, platelet producing megakaryocytes, platelets, monocytes, macrophages, dendritic cells, lymphocytes, NK cells, NKT cells, innate lymphocytes, multipotent hematopoietic progenitor cells, oligopotent hematopoietic progenitor cells, and lineage restricted hematopoietic progenitors.
65. The induced hematopoietic stem cell of paragraph 63, wherein the hematopoietic lineage cell is selected from a multi-potent progenitor cell (MPP), common myeloid progenitor cell (CMP), granulocyte-monocyte progenitor cells (GMP), common lymphoid progenitor cell (CLP), and pre-megakaryocyte-erythrocyte progenitor cell.
66. The induced hematopoietic stem cell of paragraph 63, wherein the hematopoietic lineage cell is selected from a megakaryocyte-erythrocyte progenitor cell (MEP), a ProB cell, a PreB cell, a PreProB cell, a ProT cell, a double-negative T cell, a pro-NK cell, a pro-dendritic cell (pro-DC), pre-granulocyte/macrophage cell, a granulocyte/macrophage progenitor (GMP) cell, and a pro-mast cell (ProMC).
67. The induced hematopoietic cell of any of paragraphs 49-61, wherein the stem cell is an embryonic stem cell or a progeny thereof
68. The induced hematopoietic cell of any of paragraphs 49-61, wherein the stem cell is an induced pluripotent stem cell or a progeny thereof
69. An induced hematopoietic stem cell induced by increasing or inducing in a somatic cell, a pluripotent cell, a progenitor cell or a stem cell the expression of at least one of the factors selected from the group consisting of nucleic acid encoding a gene encoding CDKN1C, DNMT3B, EGR1, ETV6, EVI1, GATA2, GFI1B, GLIS2, HLF, HMGA2, HOXA5, HOXA9, HOXB3, HOXB4, HOXB5, IGF2BP2, IKZF2, KLF12, KLF4, KLF9, LMO2, MEIS1, MSI2, MYCN, NAP1L3, NDN, NFIX, NKX2-3, NR3C2, PBX1, PRDM16, PRDM5, RARB, RBBP6, RBPMS, RUNX1, RUNX1T1, SMAD6, TAL1, TCF15, VDR, ZFP37, ZFP467, ZFP521, ZFP532, and ZFP612.
70. The induced hematopoietic stem cell of paragraph 69, wherein the increasing or inducing is performed by contacting the somatic cell, the pluripotent cell, the progenitor cell or the stem cell with at least one small molecule capable of increasing or inducing the expression of at least one of the factors of paragraph 69.
71. An induced hematopoietic stem cell made by any one of the methods of paragraphs 20-43.
72. A clone or progeny of any of the induced hematopoietic stem cells of paragraphs 48-71.
73. A differentiated progeny cell differentiated from any of the induced hematopoietic stem cells of paragraphs 48-72.

74. A hematopoietic stem cell (HSC) inducing composition comprising modified mRNA sequences encoding at least one, two, three, four, five, six, seven, eight, or more HSC inducing factors selected from: CDKN1C, DNMT3B, EGR1, ETV6, EVI1, GATA2, GFI1B, GLIS2, HLF, HMGA2, HOXA5, HOXA9, HOXB3, HOXB4, HOXB5, IGF2BP2, IKZF2, KLF12, KLF4, KLF9, LMO2, MEIS1, MSI2, MYCN, NAP1L3, NDN, NFIX, NKX2-3, NR3C2, PBX1, PRDM16, PRDM5, RARB, RBBP6, RBPMS, RUNX1, RUNX1T1, SMAD6, TAL1, TCF15, VDR, ZFP37, ZFP467, ZFP521, ZFP532, and ZFP612, wherein each cytosine of each said modified mRNA sequence is a modified cytosine, each uracil of each said modified mRNA sequence is a modified uracil, or a combination thereof
75. The HSC inducing composition of paragraph 74, wherein the at least one, two, three, four, or more HSC inducing factors are HLF, RUNX1T1, PBX1, LMO2, PRDM5, ZFP37, MYCN, MSI2, NKX2-3, MEIS1, and RBPMS.
76. The HSC inducing composition of paragraph 74, wherein the at least one, two, three, four, or more HSC inducing factors are HLF, RUNX1T1, ZFP37, PBX1, LMO2, and PRDM5
77. A hematopoietic stem cell (HSC) inducing composition comprising:
   a. a modified mRNA sequence encoding HLF;
   b. a modified mRNA sequence encoding RUNX1T1;
   c. a modified mRNA sequence encoding ZFP37;
   d. a modified mRNA sequence encoding PBX1;
   e. a modified mRNA sequence encoding LMO2; and
   f. a modified mRNA sequence encoding PRDM5;
   wherein each cytosine of each said modified mRNA sequence is a modified cytosine, each uracil of each said modified mRNA sequence is a modified uracil, or a combination thereof
78. The HSC inducing composition of paragraph 77, further comprising one or more of:
   a. a modified mRNA sequence encoding PRDM16;
   b. a modified mRNA sequence encoding ZFP467; and
   c. a modified mRNA sequence encoding VDR;
   wherein each cytosine of each said modified mRNA sequence is a modified cytosine, each uracil of each said modified mRNA sequence is a modified uracil, or a combination thereof
79. A hematopoietic stem cell (HSC) inducing composition comprising:
   a. a modified mRNA sequence encoding HLF;
   b. a modified mRNA sequence encoding RUNX1T1;
   c. a modified mRNA sequence encoding PBX1;
   d. a modified mRNA sequence encoding LMO2;
   e. a modified mRNA sequence encoding PRDM5
   f. a modified mRNA sequence encoding ZFP37;
   g. a modified mRNA sequence encoding MYCN;
   h. a modified mRNA sequence encoding MSI2;
   i. a modified mRNA sequence encoding NKX2-3;
   j. a modified mRNA sequence encoding MEIS1; and
   k. a modified mRNA sequence encoding RBPMS;
wherein each cytosine of each said modified mRNA sequence is a modified cytosine, each uracil of each said modified mRNA sequence is a modified uracil, or a combination thereof
80. A hematopoietic stem cell (HSC) inducing composition comprising:
   a. a modified mRNA sequence encoding ZFP467;
   b. a modified mRNA sequence encoding PBX1;
   c. a modified mRNA sequence encoding HOXB4; and
   d. a modified mRNA sequence encoding MSI2;
wherein each cytosine of each said modified mRNA sequence is a modified cytosine, each uracil of each said modified mRNA sequence is a modified uracil, or a combination thereof
81. The HSC inducing composition of paragraph 80, further comprising one or more of:
   a. a modified mRNA sequence encoding HLF;
   b. a modified mRNA sequence encoding LMO2;
   c. a modified mRNA sequence encoding PRDM16; and
   d. a modified mRNA sequence encoding ZFP37.
wherein each cytosine of each said modified mRNA sequence is a modified cytosine, each uracil of each said modified mRNA sequence is a modified uracil, or a combination thereof
82. A hematopoietic stem cell (HSC) inducing composition comprising:
   a. a modified mRNA sequence encoding MYCN;
   b. a modified mRNA sequence encoding MSI2;
   c. a modified mRNA sequence encoding NKX2-3; and
   d. a modified mRNA sequence encoding RUNX1T1;
wherein each cytosine of each said modified mRNA sequence is a modified cytosine, each uracil of each said modified mRNA sequence is a modified uracil, or a combination thereof
83. The HSC inducing composition of paragraph 82, further comprising one or more of:
   a. a modified mRNA sequence encoding HOXB5;
   b. a modified mRNA sequence encoding HLF;
   c. a modified mRNA sequence encoding ZFP467;
   d. a modified mRNA sequence encoding HOXB3;
   e. a modified mRNA sequence encoding LMO2;
   f. a modified mRNA sequence encoding PBX1;
   g. a modified mRNA sequence encoding ZFP37; and
   h. a modified mRNA sequence encoding ZFP521;
wherein each cytosine of each said modified mRNA sequence is a modified cytosine, each uracil of each said modified mRNA sequence is a modified uracil, or a combination thereof
84. A hematopoietic stem cell (HSC) inducing composition comprising:
   a. a modified mRNA sequence encoding HOXB4;
   b. a modified mRNA sequence encoding PBX1;
   c. a modified mRNA sequence encoding LMO2;
   d. a modified mRNA sequence encoding ZFP467; and
   e. a modified mRNA sequence encoding ZFP521;
wherein each cytosine of each said modified mRNA sequence is a modified cytosine, each uracil of each said modified mRNA sequence is a modified uracil, or a combination thereof
85. The HSC inducing composition of paragraph 84, further comprising one or more of:
   a. a modified mRNA sequence encoding KLF12;
   b. a modified mRNA sequence encoding HLF; and
   c. a modified mRNA sequence encoding EGR;
wherein each cytosine of each said modified mRNA sequence is a modified cytosine, each uracil of each said modified mRNA sequence is a modified uracil, or a combination thereof
86. A hematopoietic stem cell (HSC) inducing composition comprising:
   a. a modified mRNA sequence encoding MEIS1;
   b. a modified mRNA sequence encoding RBPMS;
   c. a modified mRNA sequence encoding ZFP37;
   d. a modified mRNA sequence encoding RUNX1T1; and
   e. a modified mRNA sequence encoding LMO2.
wherein each cytosine of each said modified mRNA sequence is a modified cytosine, each uracil of each said modified mRNA sequence is a modified uracil, or a combination thereof
87. The HSC inducing composition of paragraph 86, further comprising one or more of:
   a. a modified mRNA sequence encoding KLF12; and
   b. a modified mRNA sequence encoding HLF;
wherein each cytosine of each said modified mRNA sequence is a modified cytosine, each uracil of each said modified mRNA sequence is a modified uracil, or a combination thereof
88. A hematopoietic stem cell (HSC) inducing composition comprising:
   a. a modified mRNA sequence encoding ZFP37;
   b. a modified mRNA sequence encoding HOXB4;
   c. a modified mRNA sequence encoding LMO2; and
   d. a modified mRNA sequence encoding HLF;
wherein each cytosine of each said modified mRNA sequence is a modified cytosine, each uracil of each said modified mRNA sequence is a modified uracil, or a combination thereof
89. The HSC inducing composition of paragraph 88, further comprising one or more of:
   a. a modified mRNA encoding MYCN;
   b. a modified mRNA encoding ZFP467;
   c. a modified mRNA encoding NKX2-3
   d. a modified mRNA encoding PBX1; and
   e. a modified mRNA encoding KLF4;
wherein each cytosine of each said modified mRNA sequence is a modified cytosine, each uracil of each said modified mRNA sequence is a modified uracil, or a combination thereof
90. The HSC inducing compositions of any one of paragraphs 74-89, wherein the modified cytosine is 5-methylcytosine and the modified uracil is pseudouracil.
91. The HSC inducing compositions of any one of paragraphs 74-90, wherein the modified mRNA sequences comprise one or more nucleoside modifications selected from the group consisting of pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, 4-methoxy-2-thio-pseudouridine, 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, 2-aminopurine, 2,6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl)adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine, inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine, and combinations thereof
92. A kit for making induced hematopoietic stem cells (iHSCs) comprising the HSC inducing compositions comprising modified mRNA sequence components of any one of paragraphs 74-91.

### EXAMPLES

HSC reprogramming necessitates imparting both self-renewal potential and multi-lineage capacity onto otherwise non-self-renewing, lineage-restricted cells. Induced HSCs must also be able to interact with the stem cell niche in order to sustain productive hematopoiesis, and be able to regulate long periods of dormancy (quiescence) and yet retain the capacity to generate downstream progenitors when called into cycle. The approaches described herein permit transducing committed cells with cocktails of lentiviruses bearing multiple transcriptional factors and permit efficient combinatorial screening of thousands of combinations of these factors. Moreover, the in vivo transplantation approaches described herein, in which stem cell functional potential to be imparted onto downstream progenitors is screened, allows even rare reprogramming events to be identified due to the inherent self-selecting nature of the assay system: only cells reprogrammed to functional HSCs will be able to contribute to long-term multi-lineage reconstitution, whereas cells that are not reprogrammed will only contribute to transient reconstitution of specific lineages upon transplantation (depending upon which progenitor is used). It has been recognized that one of the challenges to reprogramming mature cells is that they are inherently stable. This is, however, not necessarily true of the populations we will first attempt to reprogram which include multi-potent, oligo-potent, and lineage-restricted progenitors in the process of differentiation. Moreover, progenitors that are developmentally proximal to HSCs are likely to be more epigenetically related and therefore more permissive to reprogramming to an induced stem cell fate. At the same time clinical translation of blood cell reprogramming to HSCs may benefit most from an ability to reprogram differentiated cell types that can be readily obtained from the peripheral blood of patients.

Identification of candidate genes that mediate HSC reprogramming necessitates a detailed knowledge not only of the gene expression profile of HSCs, but also of all downstream hematopoietic progenitor and effector cells. Towards this, we have undertaken a microarray expression profiling approach in which we compared expression profiles of highly purified HSCs to the majority of downstream cell types involved in hematopoietic differentiation (FIG. 1). Microarray analysis was performed as previously described. In total, 248 expression profiles from 40 populations were generated and compiled including unpublished and published data, in addition to datasets carefully curated from available databases (FIG. 1). All datasets were subjected to stringent quality control using the ArrayQualityMetrics package of R/Bioconductor, and data not meeting these standards were discarded. Unsupervised hierarchical clustering analysis of normalized data showed that lineal relationships and the hierarchical structure of the hematopoietic hierarchy could be recapitulated confirming the biological robustness of the data.

Although expression datasets of selected hematopoietic populations have been published, the dataset we have generated, and described herein, represents the most comprehensive database of the molecular attributes of hematopoiesis from stem cells through to effector cells available. Using this database we are readily able to identify genes specifically expressed in any hematopoietic cell type (FIG. 3). Analysis of such cell type-specific gene lists indicates that functionally important genes can be identified.

To clone HSC-enriched TFs, a cDNA library we generated from FACS purified HSCs is used, which allow cloning of splice variants that uniquely operate in HSCs. Consistent with this we have cloned splice variants for Nkx2-3, Msi2, Runx1, and Prdm16 and Zfp467 that are either minor variants, or have not been previously reported. To date, we have successfully cloned these TFs and confirmed their integrity by sequencing.

To test the viability of the approaches described herein for identifying HSC reprogramming factors, experiments were conducted in which progenitors were transduced with 22 individual TFs and evaluated by the phenotypic and functional assays detailed above. To show one example, enforced expression of HLF in MPPs (ckit⁺Sca1⁺lin⁻flk2⁺CD34⁺CD150⁻CD48⁺) or myeloid progenitors (ckit⁺Sca1⁻lin⁻CD150⁻CD48⁺) was able to endow a significant fraction of the transduced cells with a primitive CD150⁺lin⁻ surface phenotype (consistent with primitive stem/progenitor cells) over a time course of ex vivo culturing. After 30 days in culture in the presence of Dox, the cells were cytospun and stained, which revealed that the HLF-transduced cultures contained multiple cell types including megakaryocytes, macrophages, granulocytes and progenitor cells, whereas control cultures contained only macrophages. Functional evaluation in serial CFC assays showed that HLF conferred extensive self-renewal potential onto all progenitors tested. Examination of colony composition at each successive plating revealed that HLF expression led to diverse colony types including primitive CFU-GEMM. Importantly, withdrawal of Dox led to loss of both self-renewal and multi-lineage potential indicating that HLF (not insertional mutagenesis) was responsible for functional activity. Multiple independent experiments have confirmed these results. In vivo assays were then performed that demonstrated that HLF was able to endow long-term multi-lineage potential onto otherwise short-term reconstituting MPPs in transplantation assays.

FACS sorted progenitors from Rosa26-rtTA donors are transduced with cocktails of TF-bearing lentiviruses at multiplicities of infection intended to deliver multiple different viruses to individual cells. Assuming equivalence of viral titers, independence of infection, and viral titers sufficient for infecting 20% of the cells by each virus, we have calculated that to be reasonably confident of transducing each cell with at least 3 different viruses (3,276 permutations for 28 factor transductions) requires transduction of 4×10⁴ cells. This calculation does not take into account cells that are infected with more than 3 viruses, although cells transduced with more viruses can occur and may be required for reprogramming Since tens or even hundreds of thousands of downstream hematopoietic progenitors can readily be sorted from a single donor mouse, high numbers of cells can be transduced in order to maximize the chance that one or more cells is transduced with a combination of factors capable of re-establishing the stem cell state.

Different progenitor populations can be more or less amenable to reprogramming depending upon their epigenetic state and developmental proximity to HSCs. To account for this and to maximize our chances of success, FACS purified multi-potent, oligo-potent and lineage-restricted progenitors from all branches of the hematopoietic hierarchy including MPPflk2⁻, MPPflk2⁺, CLPs, Pro-B cells, Pro-T cells, CMPs, MEPs, and GMPs have been used in different experiments. Transduced progenitors (CD45.2) are transplanted into irradiated congenic (CD45.1) recipients along with a radio-protective dose of CD45.1 marrow cells to ensure survival of recipients. As noted, the lentiviral system being used is Dox-inducible, and doxycycline is administered to transplanted mice for a period of 1-4 weeks post-transplant as this should be long enough to reprogram even the most distal blood cells to HSCs. In contrast, reprogramming of blood cells to induced pluripotency takes 3 to 4 weeks.

Transplant recipients were evaluated at 4-week intervals for 24 weeks by peripheral blood analysis staining for donor-derived B-cells, T-cells and granulocytes/monocytes. Control transduced or unsuccessfully reprogrammed progenitor cells are expected to transiently reconstitute specific lineages, whereas cells successfully reprogrammed to an induced stem cell state are identified by their ability to support long-term multi-lineage reconstitution in primary recipients. In this way, the approaches described herein have a strong selection criteria for identifying reprogramming factors. Importantly, if the induced HSCs generated using the compositions and methods described herein function as endogenous HSCs do, then even the presence of a small number of induced HSCs should read out in this assay system as single HSCs can read out and be detected in transplantation assays. Thus, even if the efficiency of reprogramming is low, induced HSCs can still be identified.

To control for unintentional transplantation of contaminating HSCs from our progenitor sorts being identified as false positives, sorted progenitors were transduced with control virus and transplanted alongside test recipients. Definitive demonstration that downstream cells can be reprogrammed to HSCs can achieved when progenitors that have undergone V(D)J recombination such as Pro-B cells are used as the starting cell type, as described herein, since all blood cells derived from such induced HSCs will have, and can be screened for the recombined locus, and this can serve as a “bar code” for identifying iHSCs.

The in vivo strategies described herein are designed to screen the potential of thousands of combinations of TFs for the ability to affect reprogramming. However, since cells transfected with multiple viruses are being screened, additional steps are necessary to determine which TFs mediated activity in successful reprogramming experiments. To achieve this, donor-derived granulocytes from recipients exhibiting stable long-term multi-lineage reconstitution can be FACS sorted, DNA extracted, and TFs cloned out by factor specific PCR, as demonstrated herein. Granulocytes are used since they are short-lived and their continued production results from ongoing stem cell activity. Primer pairs for each TF have been designed and tested, as described herein.

Experiments were performed to determine the minimum complement of TFs required for reprogramming, as described herein. Removing individual TFs from subsequent transduction/transplantation experiments and then assaying for loss of reprogramming ability achieves this, as shown herein. Once a minimal set of TFs capable of reprogramming a given progenitor was determined, whether the same set of factors is also able to mediate reprogramming of different blood lineages can be tested, as described herein. Experiments have been carried out using different oligo-potent progenitor cells, and depending upon the success of these experiments, terminal effector blood cells including B-cells, T-cells, and monocyte/macrophages are tested.

A key issue related to all reprogramming studies is the efficiency with which reprogramming can be affected. To determine this, limited dilution transplantation experiments were performed with blood cells transduced with validated reprogramming factors. To do this effectively, a polycistronic lentivirus containing the core complement of reprogramming factors is constructed. Use of such a polycistronic virus is important to ensure that all cells are transduced with all factors thereby allowing an accurate determination of limit dilution frequency, and by extension, reprogramming efficiency. Primary purified HSCs are used as a control in these experiments.

In some embodiments of the compositions, methods, and kits described herein, the nucleic acid sequences encoding the HSC inducing factor(s), such as HLF, RUNX1T1, PBX1, LMO2, PRDM5, ZFP37, MYCN, MSI2, NKX2-3, MEIS1, and RBPMS, are introduced or delivered using one or more inducible lentiviral vectors. Control of expression of HSC inducing factors delivered using one or more inducible lentiviral vectors can be achieved, in some embodiments, by contacting a cell having at least one HSC inducing factor in an expression vector under the control of or operably linked to an inducible promoter, with a regulatory agent (e.g., doxycycline) or other inducing agent. When using some types of inducible lentiviral vectors, contacting such a cell with an inducing agent induces expression of the HSC inducing factors, while withdrawal of the regulatory agent inhibits expression. When using other types of inducible lentiviral vectors, the presence of the regulatory agent inhibits expression, while removal of the regulatory agent permits expression. As used herein, the term “induction of expression” refers to the expression of a gene, such as an HSC inducing factor encoded by an inducible viral vector, in the presence of an inducing agent, for example, or in the presence of one or more agents or factors that cause endogenous expression of the gene in a cell.

In some embodiments of the aspects described herein, a doxycycline (Dox) inducible lentiviral system is used. Unlike retroviruses, lentiviruses are able to transduce quiescent cells making them amenable for transducing a wider variety of hematopoietic cell types. For example, the pHAGE2 lentivirus system has been shown to transduce primary hematopoietic progenitor cells with high efficiency. This vector also carries a reporter cassette (IRES Zs-Green) that enables evaluation of viral transduction efficiencies and purification of transduced cells by FACS. The ability to inducibly turn off introduced transcription factors, as demonstrated herein, is important since the HSC-enriched expression pattern of these TFs indicates their continued enforced expression in induced HSCs can impair differentiation to all lineages. Having an inducible system also allows ascertainment of the stability of the reprogrammed state and assess the establishment and fidelity of HSC transcriptional programs and epigenetic marks once enforced expression of reprogramming factors is lifted.

As demonstrated herein, the use of polycistronic viral expression systems can increase the in vivo reprogramming efficiency of somatic cells to iHSCs. Accordingly, in some embodiments of the aspects described herein, a polycistronic lentiviral vector is used. In such embodiments, sequences encoding two or more of the HSC inducing factors described herein, are expressed from a single promoter, as a polycistronic transcript. Polycistronic expression vector systems use internal ribosome entry sites (IRES) elements to create multigene, or polycistronic, messages. IRES elements are able to bypass the ribosome scanning model of 5′-methylated Cap dependent translation and begin translation at internal sites (Pelletier and Sonenberg, 1988). IRES elements can be linked to heterologous open reading frames. Multiple open reading frames can be transcribed together, each separated by an IRES, thus creating polycistronic messages. By virtue of the IRES element, each open reading frame is accessible to ribosomes for efficient translation. Multiple genes can be efficiently expressed using a single promoter/enhancer to transcribe a single message. See, for example, U.S. Pat. Nos. 4,980,285; 5,925,565; 5,631,150; 5,707,828; 5,759,828; 5,888,783; 5,919,670; and 5,935,819; and Sambrook et al., Molecular Cloning: A Laboratory Manual, 2nd Ed., Cold Spring Harbor Press (1989).

The experiments described herein indicate that the approaches described herein are a viable approach to affect HSC reprogramming. As described herein, purified MPPs (ckit+Sca1+lin−flk2+CD34+CD150−) transduced with control, or a pool of 17 different TF viruses were transplanted into irradiated congenic recipients. As expected, MPPs transduced with control virus gave rise to long-lived B- and T-cells but their myeloid lineage potential was quickly extinguished by 8 weeks post-transplant consistent with the fact that MPPs do not self-renew. MPPs transduced with the 17-factor cocktail however gave rise to long-term myeloid, B- and T-cell reconstitution in recipient mice, indicating successful reprogramming of these progenitors to an induced HSC fate. The fact that all transplant recipients in this experiment were multi-lineage reconstituted indicates that reprogramming was not a rare event.

To rigorously test multi-potency and self-renewal, induced HSCs are FACS purified from the bone marrow (BM) of primary transplant recipients 4 months post-transplant by stringent cell surface criteria, as described herein. These cells are serially transplanted at varying doses (10, 50, 250 cells) into secondary (2°) recipients (along with radio-protective BM cells), to gauge their functional potential in comparison to endogenous, unmanipulated HSCs. Peripheral blood analysis of recipients is performed at monthly intervals for 4 months to evaluate multi-potency and long-term-self renewal. In addition, 3° and 4° transplants can be performed to establish the absolute replicative capacity of induced HSCs. BM analysis 4 months post-transplant of 1° and 2° recipients is done to determine the extent to which induced HSCs reconstitute the primitive stem cell compartment. At the same time, donor-derived myeloid, thrombo-erythroid, and lymphoid progenitor compartments are quantified to evaluate the ability of induced HSCs to give rise to different progenitor compartments.

Single HSCs that are rigorously purified are able to reconstitute irradiated recipients at a frequency of about 40% of transplant recipients. To clonally evaluate induced HSCs, single reprogrammed HSCs are sorted from the BM of primary recipients and transplanted into irradiated secondary recipients along with radio-protective BM cells, as described herein. Peripheral blood analysis of donor-chimerism is done as described above to evaluate the functional capacity of individual clones. CFC activity in methylcellulose is also used to assess clonal ability of induced HSCs. Purified unmanipulated HSCs are used as controls in these assays.

To examine the fidelity of reprogramming at the molecular level, donor-derived induced HSCs can be FACS purified from the BM of recipient mice 4 months post-transplant, as described herein, and RNA extracted, and microarray analysis performed as described. Resulting data is normalized to our hematopoietic expression database and unsupervised hierarchical clustering analysis is performed to determine the extent to which induced HSCs recapitulate the molecular signature of endogenous HSCs, as described herein. qRT-PCR analysis is performed to confirm the integrity of the microarray data as described.

Finally, stringent evaluation of reprogramming at the molecular level is best achieved by determining how faithfully epigenetic marks are re-established. To examine this, sorted induced HSCs and endogenous HSCs are subjected to genome-wide methylation analysis using reduced representation bi-sulfite deep sequencing, which provides nucleotide level resolution of CpG methylation status at genome scale.

As described herein, we have employed doxycycline to achieve relatively high levels of expression of individual TFs as measured by qRT-PCR, and reporter activity. However, successful reprogramming can require expression levels to be within a certain range. In consideration of this, doxycycline can be titered to achieve different levels of expression. Lentiviral integration can inadvertently activate genes contributing to reprogramming and in such a way confound interpretations regarding the reprogramming activity of introduced TFs. Subsequent validation experiments however can be designed to control for this.

An important consideration for the compositions and methods described herein is that induced HSCs must be capable of homing to and occupying a suitable niche to mediate long-term multi-lineage reconstitution. Transplanting transduced progenitors cells into lethally irradiated recipients can enable this homing, since irradiation acts, at least in part, to clear endogenous HSCs from their bone marrow niche facilitating occupancy by transplanted HSCs. Further, since HSCs have the ability to exit their niches, circulate, and then re-home to niches in the normal course of their biology, induced HSCs should be capable of homing to, and establishing residency in a productive niche. However, should induced HSCs fail to properly engraft within the bone marrow, alternative strategies of direct intra-femoral injection can be applied to directly deposit transduced progenitors into the bone marrow of irradiated recipients. Alternatively, co-transduction with Cxcr4, a critical HSC homing receptor can be used to facilitate proper homing of induced HSCs.

The inducible TF expression in the systems described herein require the presence of doxycycline (Dox) and the tet-transactivator, rtTA. Towards this, an rtTA lentivirus has been cloned that can be co-transduced with the TF containing viruses. We have also obtained a transgenic strain in which rtTA is constitutively expressed from the Rosa26 locus. Using cells isolated from these mice obviates the need for rtTA co-transduction. All viruses are titered using Jurkat cells. Experiments show that high titer viruses can be generated that routinely transduce purified hematopoietic progenitors with high efficiency (50-90%), and that the system is tightly Dox-inducible in vivo.

HSC inducing factors capable of reprogramming progenitors to an HSC state can be capable of introducing phenotypic properties of HSCs onto transduced progenitors through continued enforced expression. To evaluate this, TF-transduced progenitors were monitored for markers associated with HSCs by flow cytometry during ex vivo culturing. Experiments can first be conducted using single TF-transductions, followed by experiments in which TFs are co-transduced. For these experiments FACS purified progenitors are transduced for 2 days with virus followed by resorting the transduced cells (Zs-Green positive). 200-500 cells are seeded into wells for culturing in an HSC supportive media. Flow cytometry is performed at weekly intervals for a month Immunostaining of cells can be performed with antibodies for CD150, and lineage markers (cocktail of antibodies against differentiated cells) since these have been shown to be reliable for HSC identification under diverse conditions. Transcription factors scoring positively with these markers can be examined using additional HSC markers including Sca1, CD48, CD105 and CD20127. On day 30, cultures are cytospinned, stained (May-Grunwald), and cell types scored.

Depending upon which starting cell is being reprogrammed, in some embodiments, it can be required to knockdown lineage specific factors to convert downstream progenitors back to an induced HSC fate, such as, for example when using B-lineage committed cells.

TABLE 5
Primer Sequences Used For Reverse Cloning of HSC Inducing Factors

| | | SEQ ID | | SEQ ID | |
|---|---|---|---|---|---|
| Factor | 5′ Primer | NO: | 3′ Primer | NO: | Size |
| Hoxb5 | CCTGTCCTCGCCCGAGTCCCT | 94 | CGTCGCCGCCGGGTCAGG | 131 | 465 |
| | GCC | | TAGCGATTG | | |
| Rarb | CTCGTCCCGAGCCCACCATC | 95 | GCAAAGGTGAACACAAG | 132 | 696 |
| | TCCACTTCCTCC | | GTCAGTCAGAGG | | |
| Ndn | CAACAACCGTATGCCCATGA | 96 | CATCCTCTTCTGGTCCTTC | 133 | 275 |
| | CAGG | | ACCAAC | | |
| Evi1 | GGAGGTGGGATGGAGGGAA | 97 | CAATTTCATCGGGAACAG | 134 | 313 |
| | TCCTTG | | CAACCATG | | |
| Nap1l3 | GGGAAATTGAAGTCCAGCCA | 98 | CTGCACCCGATTTCTTACG | 135 | 1000 |
| | AGAGTG | | GCTTG | | |
| Mycn | CCCGGTGAACAAGCGAGAGT | 99 | GTTGACGCTCCAGGATGT | 136 | 385 |
| | CGGCGTC | | TGTGGTTG | | |
| Meis1 | GCATGGGTTCCTCGGTCAAT | 100 | GTCCTTATCAGGGTCATC | 137 | 622 |
| | GACG | | ATCGTC | | |
| Hlf | GCGCCCTCGGTCATGGATCT | 101 | CCATGTTGTTCTTTCTGCG | 138 | 354 |
| | CAGC | | CCTCGCCC | | |
| Rbpms | GACCCTATTTGTCAGCGGTC | 102 | GAAAGCGGCAGGAGGAG | 139 | 432 |
| | TGCCTC | | GAAGAGC | | |
| Msi2 | CTCCAGAGGCTTCGGTTTCG | 103 | CTGCCATAGGTTGCCACA | 140 | 503 |
| | TCAC | | AAGTTG | | |
| Irf6 | GTGGAGACCGGAAAGTACCA | 104 | GTTTGCCCATACTCCTTCC | 141 | 535 |
| | GGAAGG | | CACGATAC | | |
| Prdm16 | GGAGGCCGACTTTGGATGGG | 105 | CTTCTCGTTGGTGATATGC | 142 | 510 |
| | AGCAG | | TCTGGACCTG | | |
| Zfp467 | GGATGGGTTCAGTAATGCCC | 106 | CCACCCGGACAGCGCGAT | 143 | 375 |
| | AGGAGAAG | | TCCACC | | |
| Zfp37 | CAGGTTTAGATGGAGTACGG | 107 | GCAAGGCCCAAGACAGCA | 144 | 506 |
| | CAGTGTG | | GGAACAAG | | |
| Vdr | CATCACCAAGGACAACCGGC | 108 | CAGCATGGAGAGCGGAG | 145 | 465 |
| | GACAC | | ACAGGTC | | |
| Nkx2-3 | CGAGGAAGAAGAGGGAGAG | 109 | CTGCCGCTGTCTCTTGCAC | 146 | 432 |
| | AAACTGTC | | TTGTACC | | |
| Zfp612 | GGTGACCTTTGAGGACGTGG | 110 | GACTAAACAAACACCCTT | 147 | 433 |
| | CTGTG | | CCACAGAGC | | |
| Runx1t1 | CAACGGGCCTTCTTCTTCCTC | 111 | CATTATTTGGACTGTACC | 148 | 533 |
| | TTCCTC | | GCTGGCCTGG | | |
| Runx1 | CTGCTCCGTGCTACCCACTC | 112 | GAGGCTGAGGGTTAAAGG | 149 | 496 |
| | ACTG | | CAGTGGAG | | |
| Hoxb4 | CGATTACCTACCCAGCGACC | 113 | CGTCAGGTAGCGATTGTA | 150 | 483 |
| | ACTC | | GTGAAACTCC | | |
| Nr3c2 | CCAACACTTGAGTTCCTTTCC | 114 | GCAGGACAGTTCTTTCTC | 151 | 405 |
| | GCCTGTC | | CGAATC | | |
| Tcf15 | CCGAAAGCTGTCTAAGATCG | 115 | CTGCCCCCCAGGTCACGA | 152 | 331 |
| | AGACG | | CGGCTGC | | |
| Hoxa5 | GGCAGCACCCACATCAGCAG | 116 | CGCCGAAGAAGGATCGAA | 153 | 291 |
| | CAGAG | | ATAGCTC | | |
| Hoxb3 | CTGGATGAAAGAGTCGAGGC | 117 | GGTAGTTGGAAGGCAGCG | 154 | 318 |
| | AAAC | | CGTAGGC | | |
| Pbx1 | GAGTTTGGATGAAGCGCAGG | 118 | GATGCCGCACTTCTTGGC | 155 | 433 |
| | CCAG | | TAACTC | | |
| Klf2 | CAAGGGTCTCCAAACGTCCA | 119 | GTCACATTTGGCAGGTCA | 156 | 605 |
| | CAAC | | TCATCG | | |
| Lmo2 | GCCATCGAAAGGAAGAGCCT | 120 | CCACTCGTAGATGTCTTGT | 157 | 443 |
| | GGAC | | TCACACAC | | |
| Etv6 | GAGCAGAGATGACGTAGCCC | 121 | GTGGTTGTTCTCCTGCTGT | 158 | 507 |
| | AGTG | | AGCCTGG | | |
| Hoxa9 | CGCTCTCCTTCGCGGGCTTAC | 122 | GTGGAGCGAGCATGTAGC | 159 | 239 |
| | CCTCC | | CAGTTGG | | |
| Igf2BP2 | GAACTGGGCCATCCGCGCCA | 123 | CTTCAGGTTTCTGCCTTCT | 160 | 703 |
| | TCGAGAC | | TTGCCAATC | | |
| Gata2 | GTCTTCTTCAACCATCTCGAC | 124 | GGTATCGGGTGGTGTGTT | 161 | 574 |
| | TCGCAGG | | GCAGGCTGGG | | |
| Zfp521 | GGGTTTCGTTGTGTGGTGTGT | 125 | GAACAAACACTGTGAAAC | 162 | 406 |
| | ATGCAG | | AGACGGG | | |
| Glis2 | CGGCAGCGGGAAGGTGAAC | 126 | GCACAGGGTGAGGAGGA | 163 | 488 |
| | GGGAGCTAC | | GGCTGAAGAG | | |
| Zfp532 | CGGTCCCGGCAGACCAGATG | 127 | CTCCTCCTCCTCATCGTTG | 164 | 518 |
| | ATAGTTC | | GTAACATC | | |
| Nfix | GCACGAGAAGCGGATGTCAA | 128 | CACATCATCTACTGGACT | 165 | 723 |
| | AGGACGAG | | CTCCATCTC | | |
| Prdm5 | CTGATGTGGGAGGTACGTGG | 129 | CAGGCAAAGTCCTCTTCA | 166 | 314 |
| | GAGCAAG | | CAGCCAAGG | | |
| Egr1 | GAGCGAGGACCAGTCACTAT | 130 | CCATATTCTTTCACCGCCC | 167 | 416 |
| | TTGAG | | ACTCC | | |

Homo sapiens hepatic leukemia factor (HLF), mRNA (SEQ ID NO: 9) and a codon optimized, or different codons encoding the same amino acids, are naturally also contemplated to be covered by the reference to the nucleic acid as set forth herein.

Homo sapiens LIM domain only 2 (rhombotin-like 1) (LMO2), transcript variant 1, mRNA (SEQ ID NO: 21) and a codon optimized, or different codons encoding the same amino acids, are naturally also contemplated to be covered by the reference to the nucleic acid as set forth herein.

Homo sapiens Meis homeobox 1 (MEIS1), mRNA (SEQ ID NO: 22) and a codon optimized, or different codons encoding the same amino acids, are naturally also contemplated to be covered by the reference to the nucleic acid as set forth herein.

Homo sapiens musashi RNA-binding protein 2 (MSI2), transcript variant 1, mRNA (SEQ ID NO: 23) and a codon optimized, or different codons encoding the same amino acids, are naturally also contemplated to be covered by the reference to the nucleic acid as set forth herein.

Homo sapiens v-myc myelocytomatosis viral related oncogene, neuroblastoma derived (avian) (MYCN), mRNA (SEQ ID NO: 24) and a codon optimized, or different codons encoding the same amino acids, are naturally also contemplated to be covered by the reference to the nucleic acid as set forth herein.

Homo sapiens NK2 homeobox 3 (NKX2-3), mRNA (SEQ ID NO: 28) and a codon optimized, or different codons encoding the same amino acids, are naturally also contemplated to be covered by the reference to the nucleic acid as set forth herein.

Homo sapiens pre-B-cell leukemia homeobox 1 (PBX1), transcript variant 2, mRNA (SEQ ID NO: 30) and a codon optimized, or different codons encoding the same amino acids, are naturally also contemplated to be covered by the reference to the nucleic acid as set forth herein.

Homo sapiens PR domain containing 5 (PRDM5), mRNA (SEQ ID NO: 32) and a codon optimized, or different codons encoding the same amino acids, are naturally also contemplated to be covered by the reference to the nucleic acid as set forth herein.

Homo sapiens RNA binding protein with multiple splicing (RBPMS), transcript variant 3, mRNA (SEQ ID NO: 35) and a codon optimized, or different codons encoding the same amino acids, are naturally also contemplated to be covered by the reference to the nucleic acid as set forth herein.

Homo sapiens runt-related transcription factor 1; translocated to, 1 (cyclin D-related) (RUNX1T1), transcript variant 5, mRNA (SEQ ID NO: 37) and a codon optimized, or different codons encoding the same amino acids, are naturally also contemplated to be covered by the reference to the nucleic acid as set forth herein.

Homo sapiens ZFP37 zinc finger protein (ZFP37), mRNA (SEQ ID NO: 42) and a codon optimized, or different codons encoding the same amino acids, are naturally also contemplated to be covered by the reference to the nucleic acid as set forth herein.

### Example 2

### Identification of Factors Capable of Imparting Alternative Lineage Potential In Vitro and Multi-Lineage Engraftment Potential on Committed Progenitors In Vivo

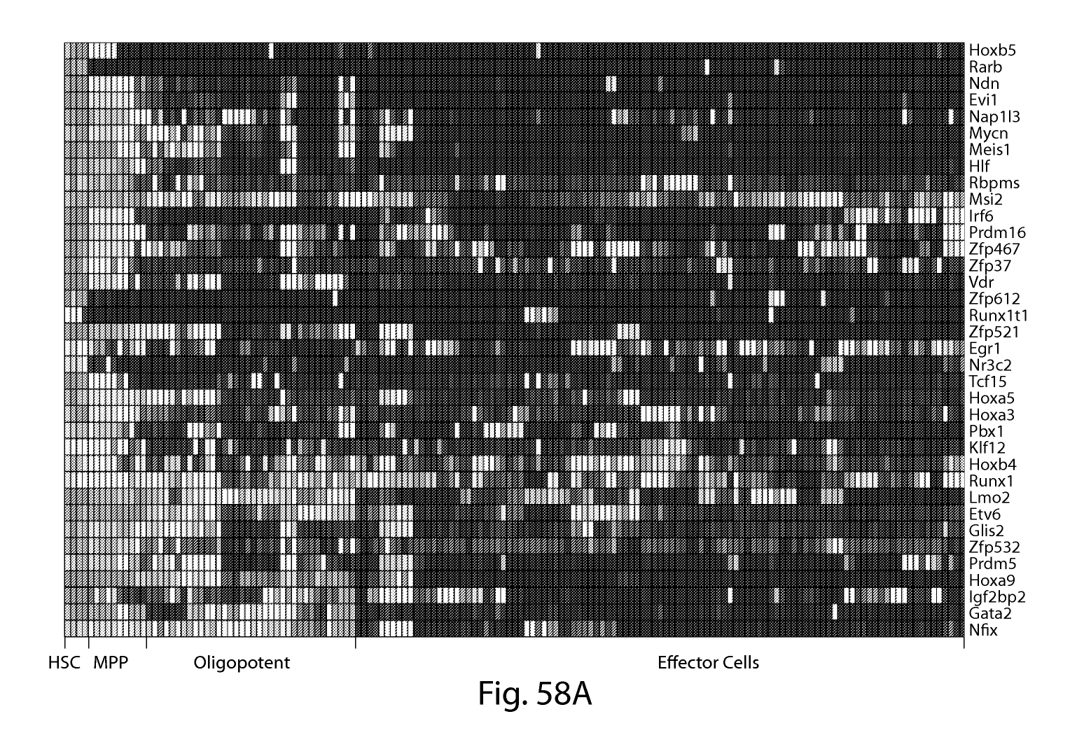

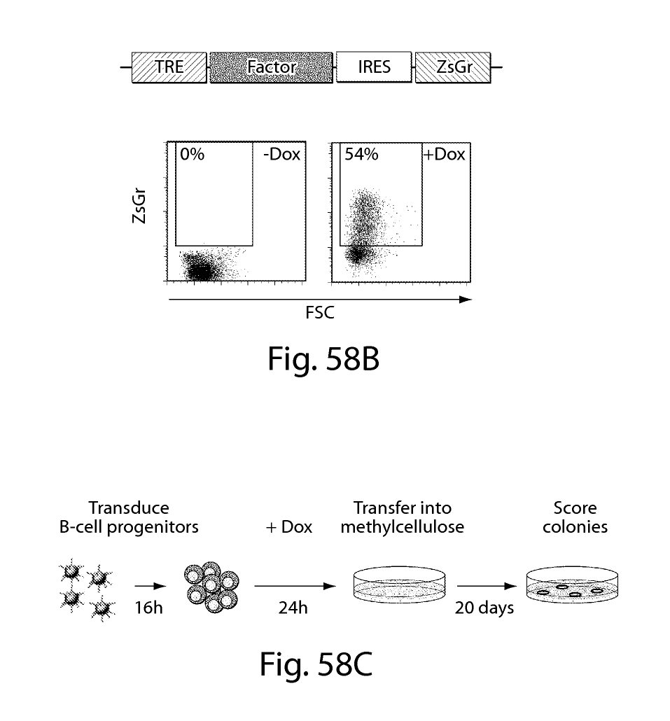

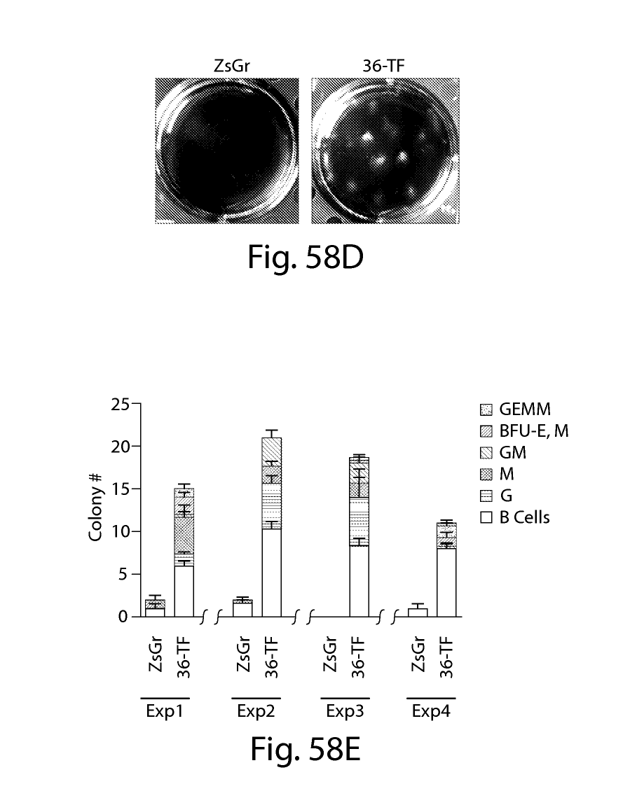

Experimental strategies for reprogramming diverse cell types generally rely on the action of one or more genes able to impart the cellular and molecular properties of one cell type onto a different cell type. We hypothesized that regulatory factors with relatively restricted expression in HSCs in relation to their downstream hematopoietic progeny are likely to be involved in defining the functional identity of HSCs through regulation of the gene networks underlying their fundamental properties which include self-renewal and multi-lineage differentiation potential. We reasoned that transient ectopic expression of such factors in committed blood cells might therefore instill them with the functional properties of HSCs and potentially stably reprogram them back to an HSC-like state. To identify such factors we analyzed microarray data of 40 different purified hematopoietic cell types that we and others have generated that comprise the vast majority of hematopoietic progenitor and effector cells in addition to HSCs. These datasets (142 arrays in total) were normalized together into a single database providing a comprehensive molecular overview of hematopoiesis from stem cells through to effector cells. Using this database we identified 36 regulatory factors with relatively restricted expression in HSCs in relation to their downstream progeny. These included 33 genes encoding transcription factors, and 3 genes encoding translational regulators (FIG. 58A). Consistent with our hypothesis, multiple genes with known roles in regulating the core properties of HSCs were identified which included Ndn (Kubota et al., 2009), Evil (Yuasa et al., 2005), Meis1 (Hisa et al., 2004), HLF (Gazit et al.), Egrl (Min et al., 2008) and others. We also identified multiple regulatory proteins that remain unstudied in HSC biology. Each of the 36 factors was then cloned into doxycycline-inducible lentiviruses bearing a reporter cassette (Zs-Green) (Mostoslaysky et al., 2005) and high-titer viruses were produced (FIG. 58B).

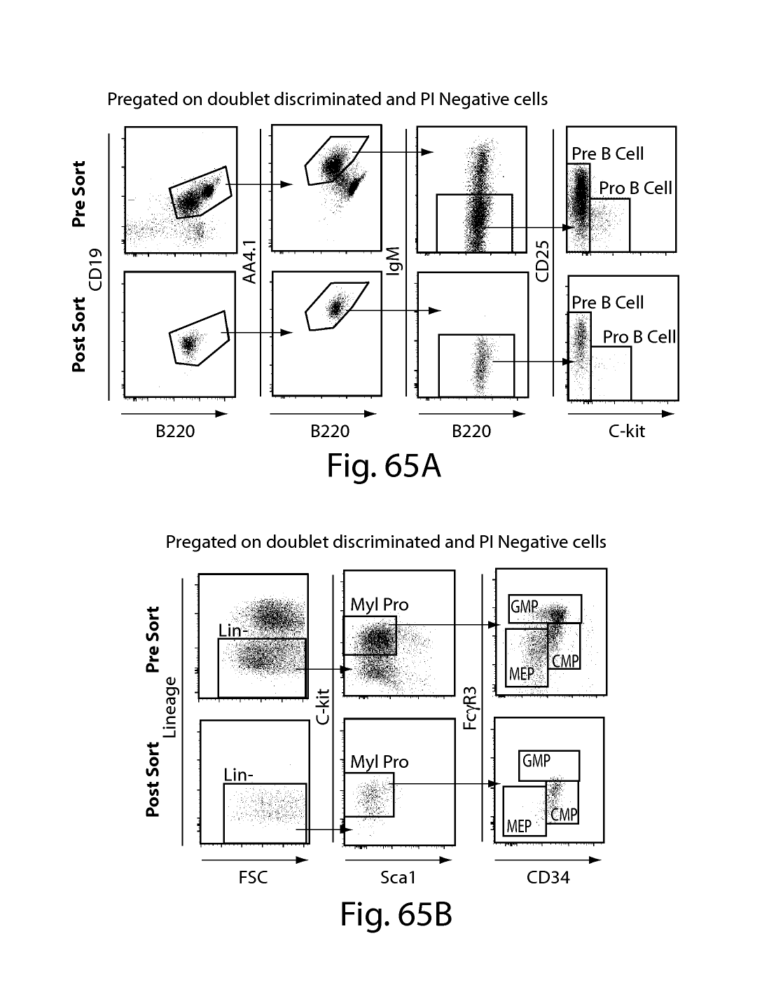

It has been recognized that one of the challenges to reprogramming mature cells is that they are inherently stable (Zhou and Melton, 2008). This is not necessarily true of oligo-potent and lineage-committed hematopoietic progenitors, which are transient cell types in the process of differentiation. Moreover, since progenitor cells proximal to HSCs are more epigenetically related to HSCs (Bock et al., 2012), we reasoned that these might be more amenable to reprogramming back to an HSC-like state. Thus we first sought to determine if we could impart alternative lineage potentials onto lineage-restricted progenitors by assaying the ability of the 36 factors to instill myeloid lineage potential onto otherwise B-cell restricted progenitors in colony forming assays. We purified Pro/Pre B-cells (CD19+B220+AA4.1+IgM−) from mice expressing the reverse tetracycline-controlled transactivator (rtTA) from the Rosa26 locus (Rosa26rtTA) (FIG. 65), and transduced them with control virus (Zs-green), or the 36-factor viral cocktail. Transduced cells were then exposed to doxycycline followed by plating into methylcellulose in the presence of myeloid promoting cytokines (FIG. 58C). These experiments showed that whereas control-transduced Pro/Pre B-cells were unable to form myeloid colonies as expected, cells transduced with the 36-factor cocktail readily gave rise to colonies bearing diverse myeloid lineages including granulocytes, erythrocytes, megakaryocytes and macrophages (FIG. 58D-E).

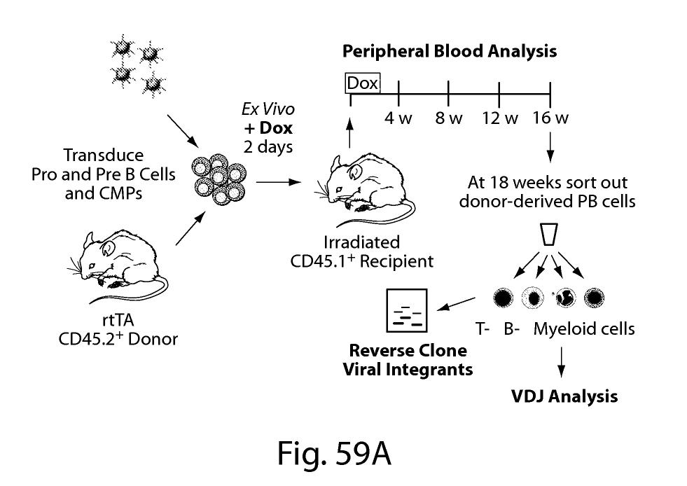

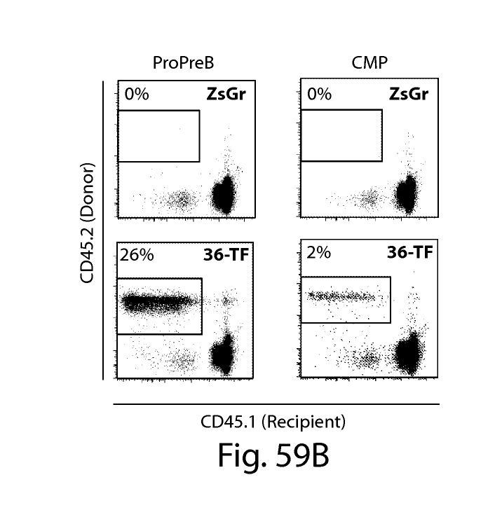

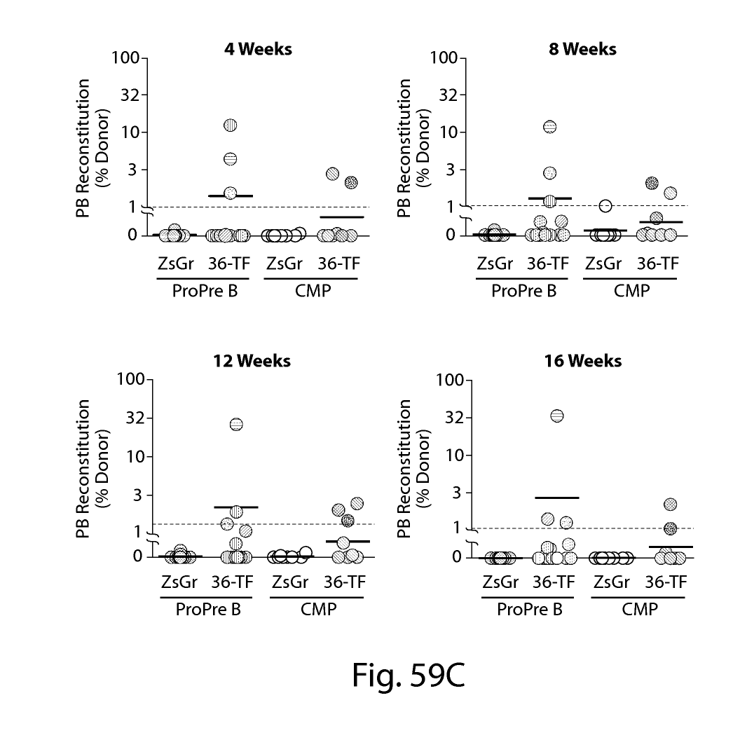

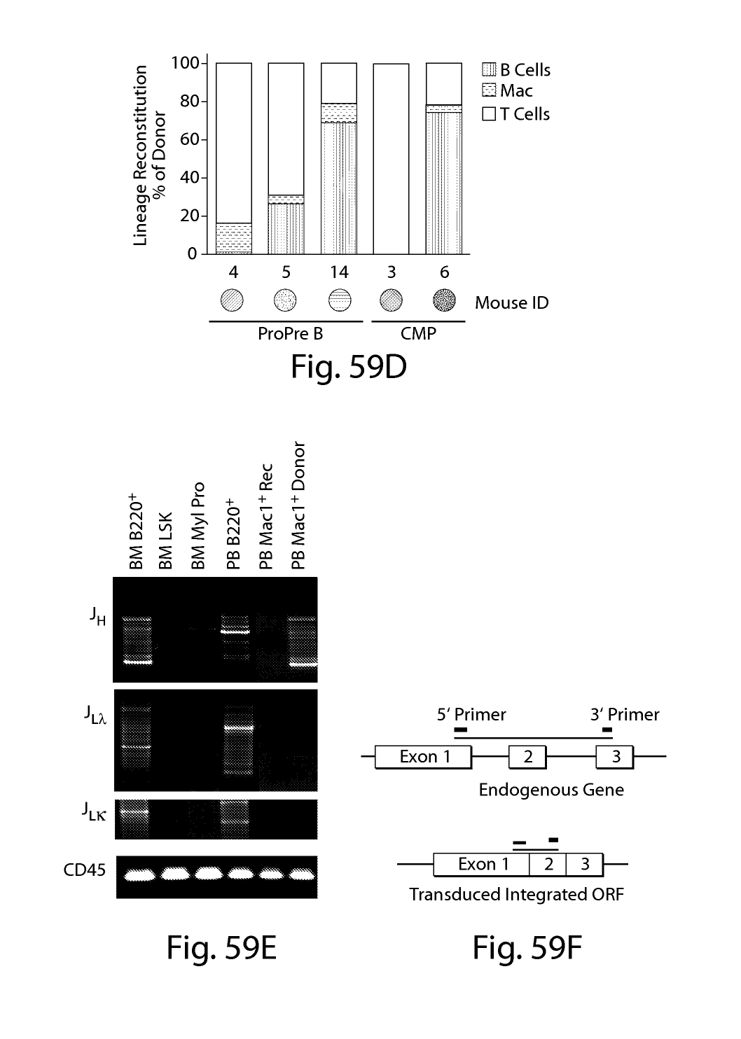

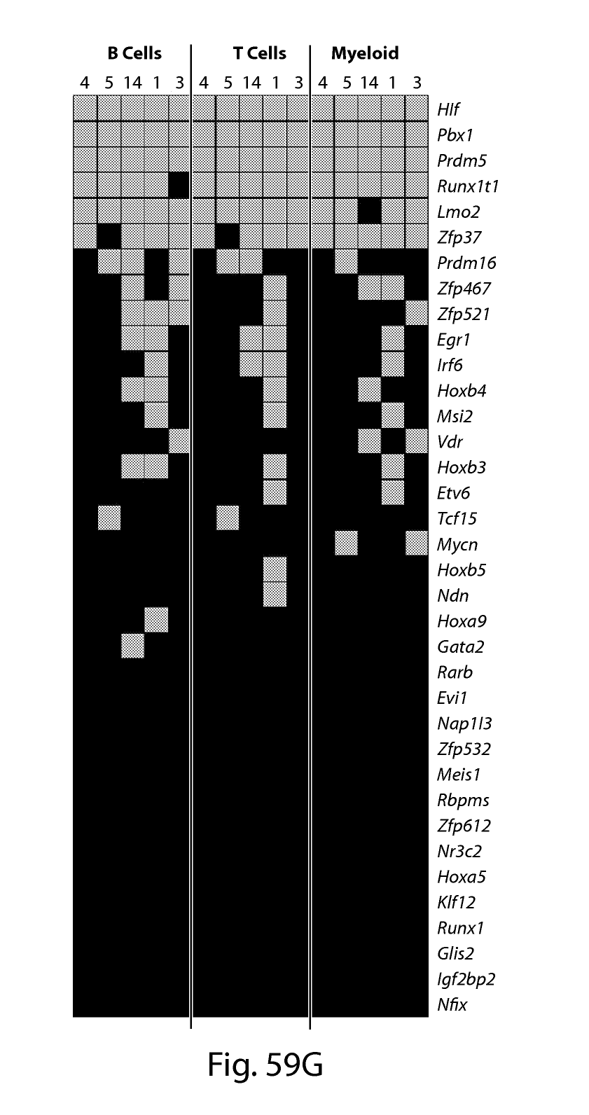

We next determined if transient ectopic expression of the 36-factor cocktail imparted HSC-like potential onto lineage-restricted lymphoid or myeloid progenitors in vivo. We took advantage of the fact that HSCs are the only hematopoietic cells capable of long-term multi-lineage reconstitution in myeloablated recipients upon transplantation, whereas downstream progenitors only transiently reconstitute recipient mice with restricted lineage potential depending upon their stage of differentiation (FIG. 59A). Moreover, we reasoned that the sensitivity of the transplantation assay, in which even a single HSC can give rise to detectable multi-lineage engraftment, would permit detection of even rare reprogramming events. Thus, only progenitors transduced with a combination of factors capable of instilling them with long-term reconstitution potential would be read out in this assay. Towards this we purified Pro/Pre B-cells or common myeloid progenitors (CMPs: lin−c-kit+Sca1−Fc□rlowCD34+) from Rosa26rtTA mice (CD45.2) and following a 2-day transduction protocol with control (Zs-green) or viruses bearing the 36-factors in the presence of doxycycline, we transplanted them into lethally irradiated congenic recipients (CD45.1) along with radio-protective bone marrow cells (CD45.1) (FIG. 59A). Doxycycline was maintained in the drinking water for 2 weeks post-transplant to maintain ectopic expression of the introduced factors, followed by doxycycline withdrawal. Peripheral blood analysis of the reconstituted mice over the 16-week course of the experiment revealed that, as expected, control-transduced Pro/Pre B-cells or CMPs did not give rise to donor-derived long-term engraftment (FIG. 59B-C). By contrast, a few of the recipients transplanted with the 36-factor transduced B-cell progenitors (3/15) or CMPs (2/8) exhibited long-term donor-derived reconstitution (FIG. 59B-C). All but one of the reconstituted mice showed multi-lineage engraftment of B-, T- and myeloid cells though the degree of engraftment of each lineage varied amongst the different recipients (FIG. 59D). Analysis of V(D)J recombination of sorted donor-derived myeloid cells from the Pro/Pre B-cell arm of the experiment confirmed the B-lineage origin of the reconstituting cells as evidenced by recombination of the heavy chain of the IG locus (FIG. 59E). The observation of multiple heavy chain bands in the gel indicated that the reconstituting cells were polyclonal.

These experiments indicated that one or more factors from the 36-factor cocktail could imbue long-term multi-lineage reconstituting potential onto otherwise committed lymphoid and myeloid progenitors. To determine which factors might be involved in conferring this potential, we sorted donor-derived myeloid, B-cells and T-cells to test for the presence of each of the 36 factors using a PCR-based strategy (FIG. 59F, Table 5). This analysis revealed that whereas multiple factors could be identified in the donor-derived cells from each of the reconstituted mice, 6 transcription factors, Hlf, Runx1t1, Pbx1, Lmo2, Zfp37, and Prdm5 were consistently detected in all of the reconstituted recipients in multiple lineages (FIG. 59G).

Six transcription factors (Hlf, Runx1t1, Pbx1, Lmo2, Zfp37, and Prdm5) are sufficient to reprogram progenitor potential in vitro and impart long-term multi-lineage engraftment potential in vivo.

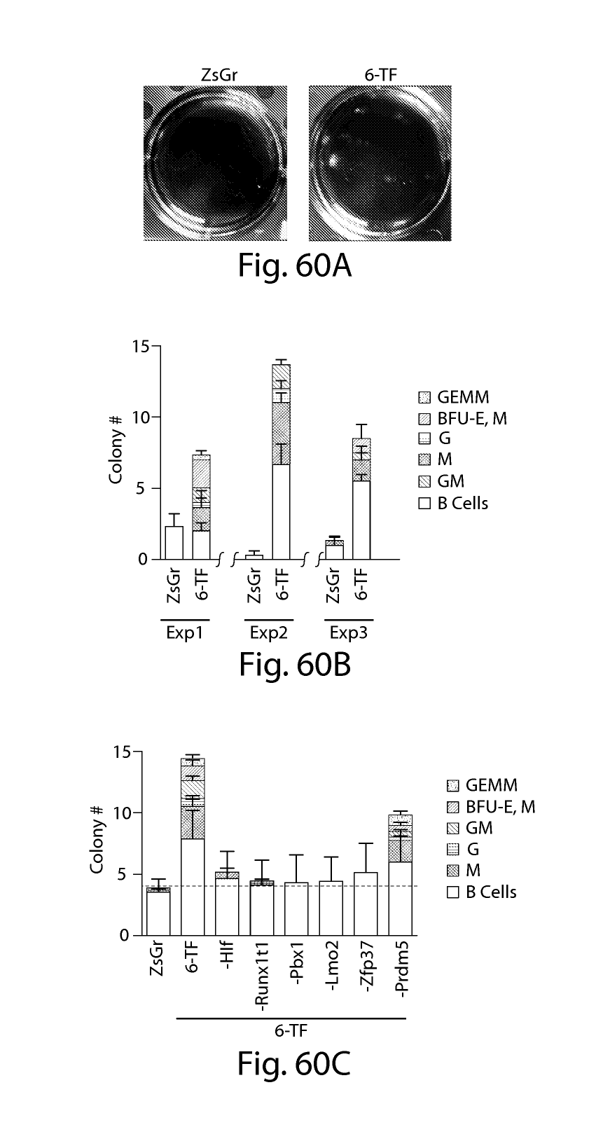

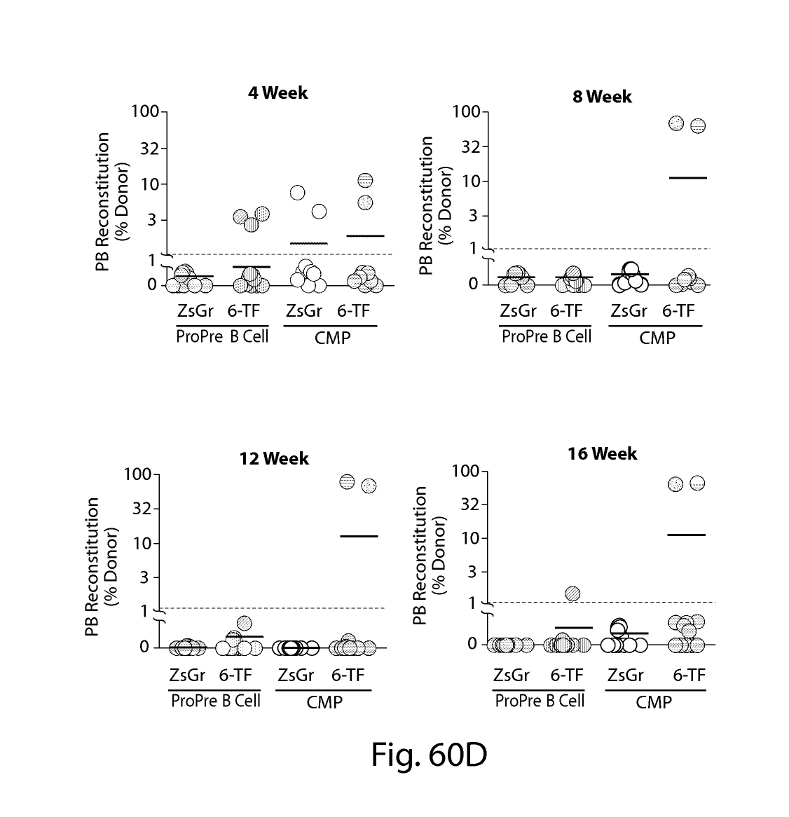

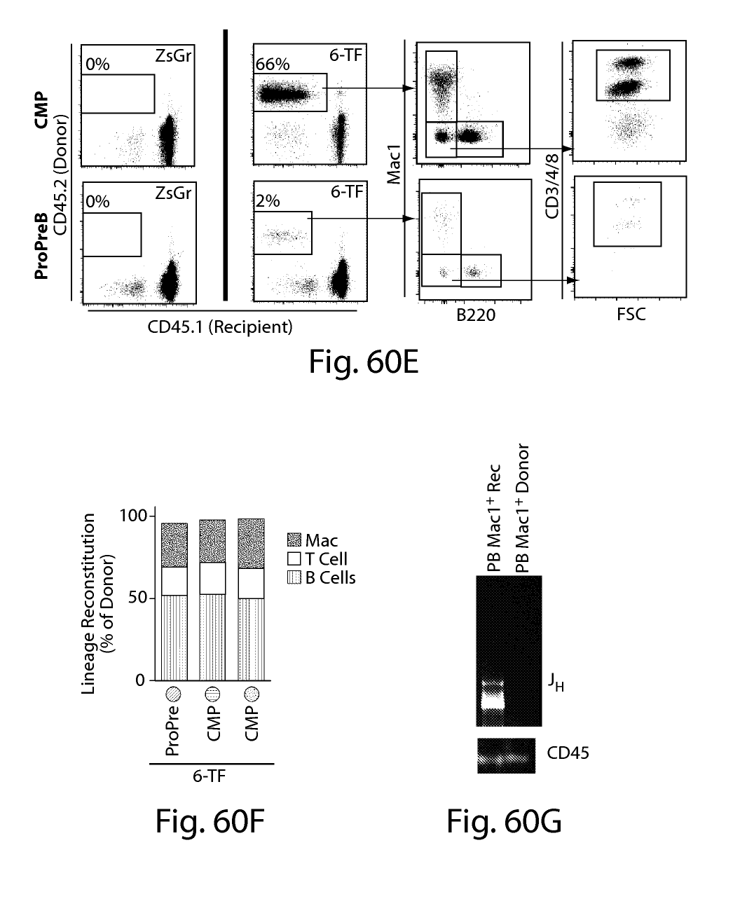

We next assessed if the 6 transcription factors we had identified in our in vivo screen were sufficient to confer myeloid colony forming potential onto Pro/Pre B-cells in methylcellulose. As we had observed with the 36-factor cocktail (FIG. 58D-E), transduction with the viral combination of Hlf, Runx1t1, Pbx1, Lmo2, Zfp37, and Prdm5 was able to imbue lineage-restricted B-cell progenitors with myeloid lineage potential in these assays (FIG. 60A-B). To test the requirement for each of the 6 transcription factors (6-TF) we employed “N minus 1” experiments in which each of the factors was sequentially omitted from the transduction cocktail (FIG. 60C). These experiments revealed that whereas Hlf, Runx1t1, Pbx1, Lmo2, and Zfp37 were all required for instilling myeloid colony forming potential onto Pro/Pre B-cells in vitro, the 5-factor cocktail minus Prdm5 still gave rise to myeloid colonies albeit at lower numbers than the 6-TF combination (FIG. 60C).

We next tested whether the 6-TF cocktail was sufficient to impart long-term multi-lineage reconstituting potential onto committed myeloid or B-cell progenitors in transplantation assays. Purified Pro/Pre B-cells (CD45.2) were transduced with control (Zs-green) virus or the 6-TF cocktail followed by transplantation into congenic recipients (CD45.1). In contrast to control-transduced cells, long-term multi-lineage reconstitution was observed in 1/13 and 2/12 recipients transplanted with 6-TF transduced Pro/Pre cells or CMPs cells, respectively (FIG. 60D). Peripheral blood analysis of recipient mice throughout the course of the experiment revealed that in all cases, donor-derived cells from the reconstituted recipients showed multi-lineage engraftment (FIG. 60D-F). Heavy chain rearrangement was observed in donor-derived myeloid cells sorted from the Pro/Pre B-cell reconstituted mouse confirming the B-cell origin of the reconstituting cells (FIG. 60G). These results indicate that transient ectopic expression of Hlf, Runx1t1, Pbx1, Lmo2, and Zfp37, and Prdm5 is sufficient to impart long-term, multi-lineage transplantation potential onto otherwise committed myeloid and lymphoid progenitors.

Inclusion of Meis1 and Mycn and use of polycistronic viruses improves in vivo reprogramming efficiency.

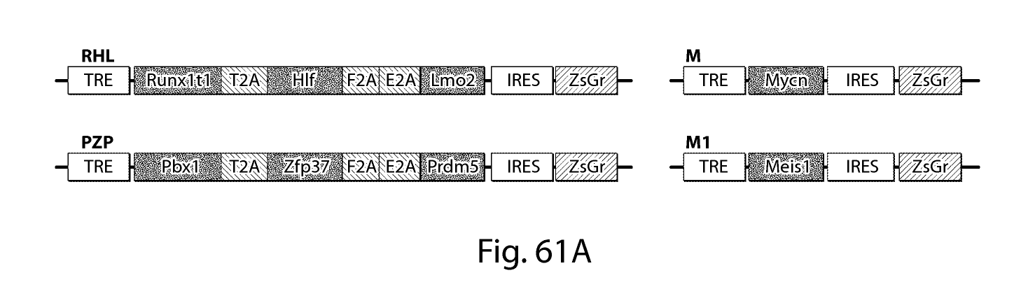

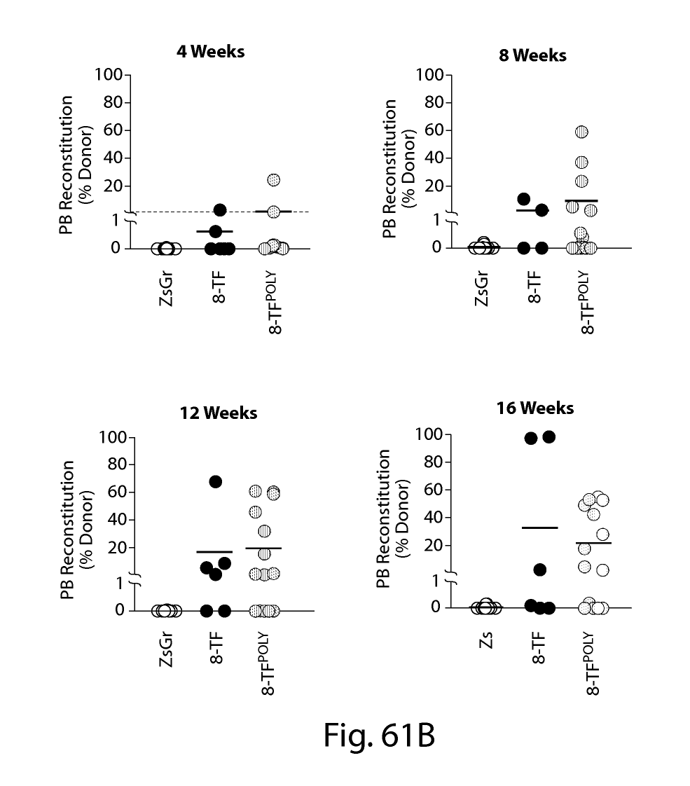

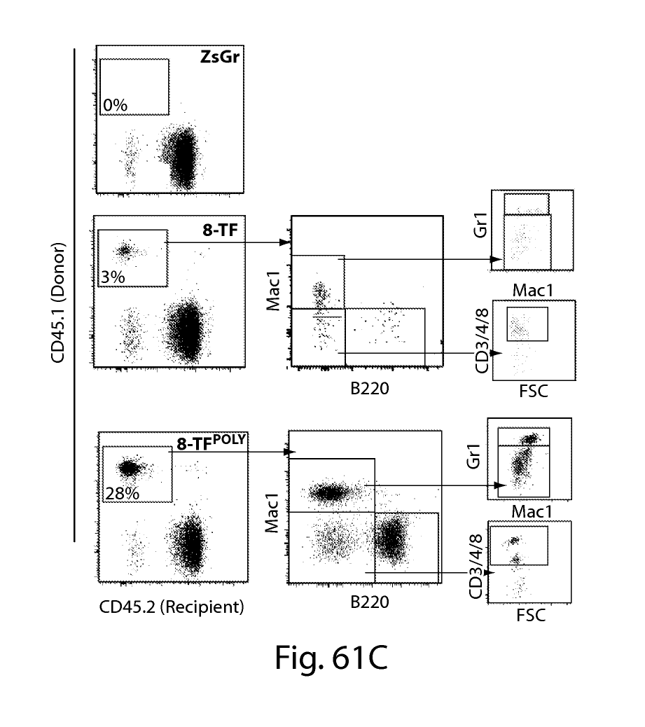

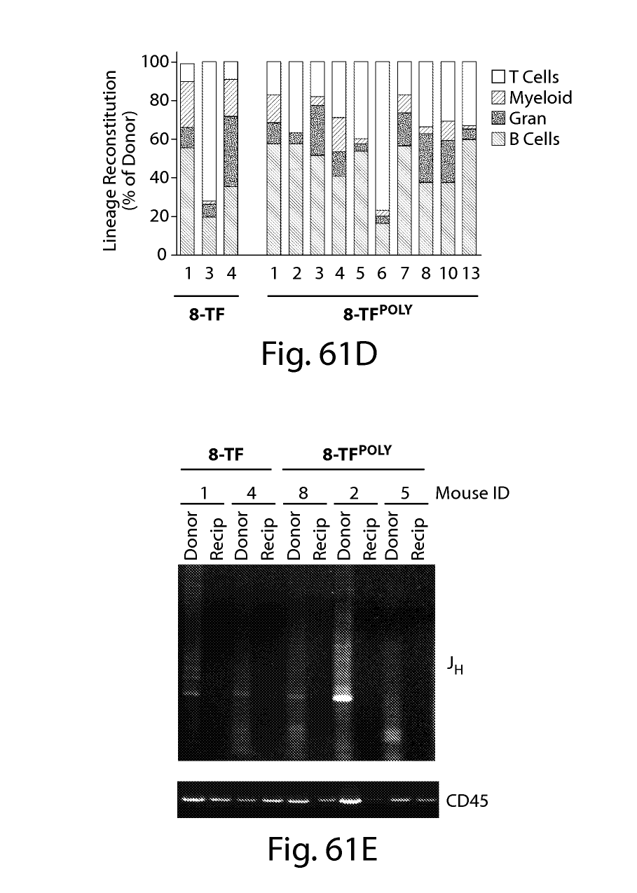

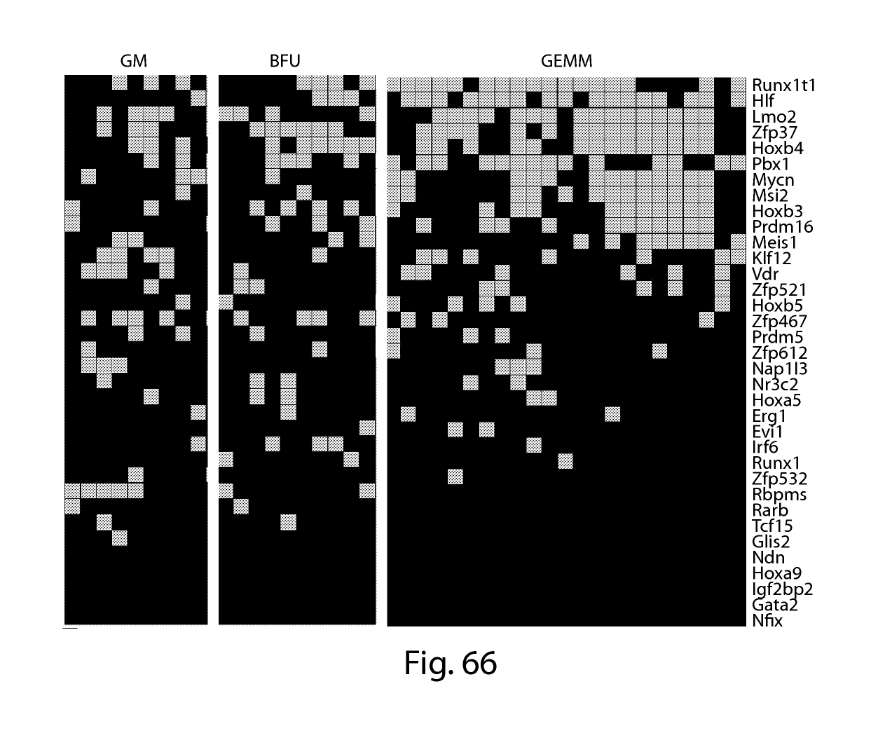

The absence of donor-derived reconstitution in many of the recipient mice in our 6-TF transplantation experiments (FIG. 60D) suggested that the efficiency of imparting this long-term multi-lineage potential onto committed progenitors was low. To try to improve this we developed polycistronic doxycycline-inducible lentiviruses bearing three transcription factors each separated by 2A peptide sequences (Runx1T1.Hlf.Lmo2 (RHL), Pbx1.Zfp37.Prdm5 (PZP)). We also included two additional transcription factors (Mycn and Meis1) that we had repeatedly identified from primitive colonies generated in in vitro colony forming experiments (FIGS. 61A, 66, and data not shown). To test the utility of these strategies we transduced purified Pro/Pre B-cells with control virus, or the 8-transcription factor cocktail as individual viruses (8-TF), or using the RHL and PZP polycistronic viruses along with viruses bearing Mycn, and Meis1 (8-TFPoly), and transplanted them into irradiated congenic recipients at greater numbers than in previous experiments. Peripheral blood analysis of transplanted mice over the course of 16 weeks revealed that in contrast to the control-transduced cells that showed no donor-derived chimerism (0/12), multiple recipients transplanted with either the 8-TF (3/6) or the 8-TFPoly (9/14) transduced cells exhibited donor-derived chimerism (FIG. 61B). All recipients showed multi-lineage reconstitution 18-22 weeks post-transplant though again the degree of B-cell, T-cell and myeloid chimerism varied amongst recipients (FIG. 61C-D). The B-cell origin of the reconstituting cells was confirmed through evidence of IG heavy chain rearrangement in donor-derived myeloid cells, with the presence of many bands indicating that the reconstituting cells were polyclonal (FIG. 61E).

Reprogrammed cells engraft bone marrow progenitor compartments and can reconstitute secondary recipients.

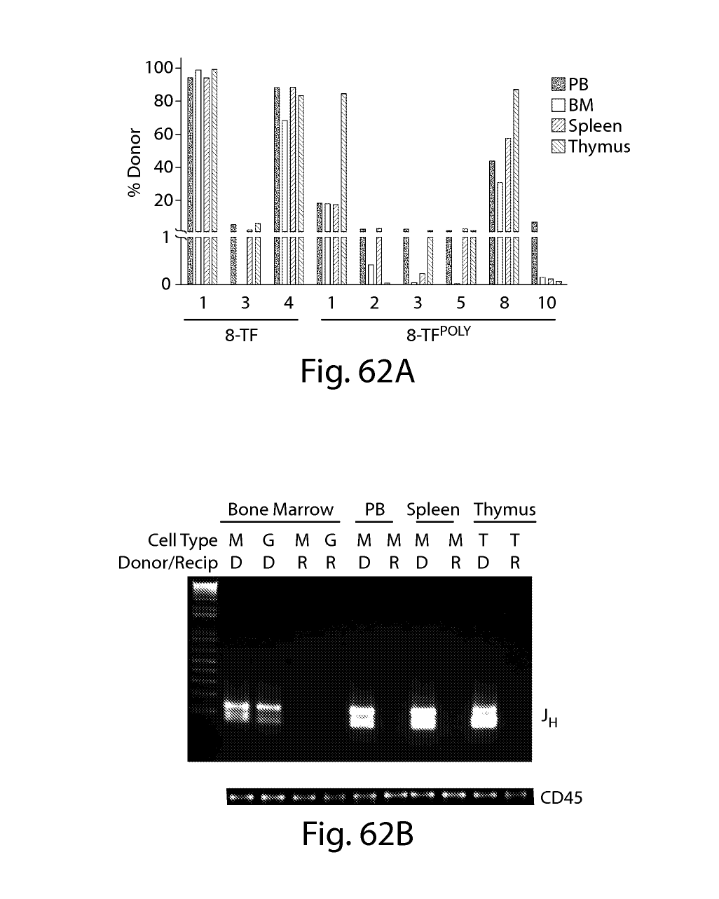

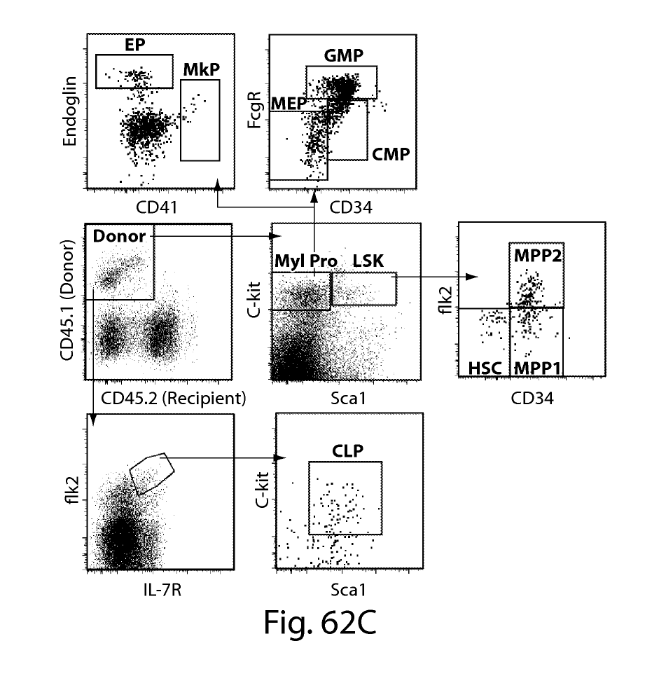

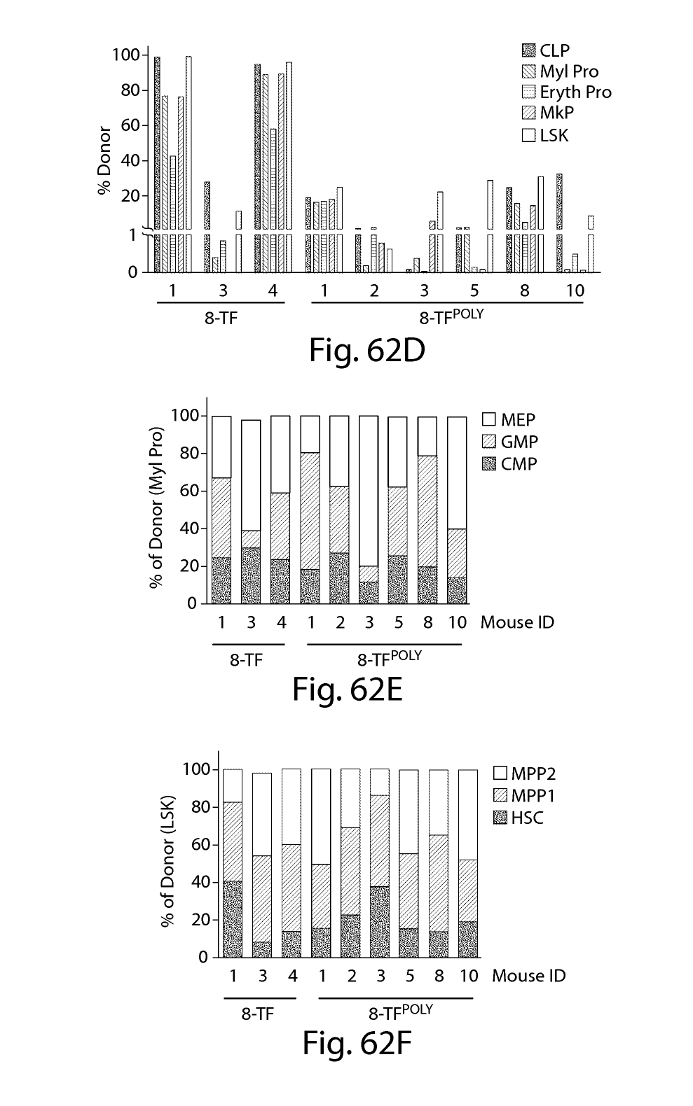

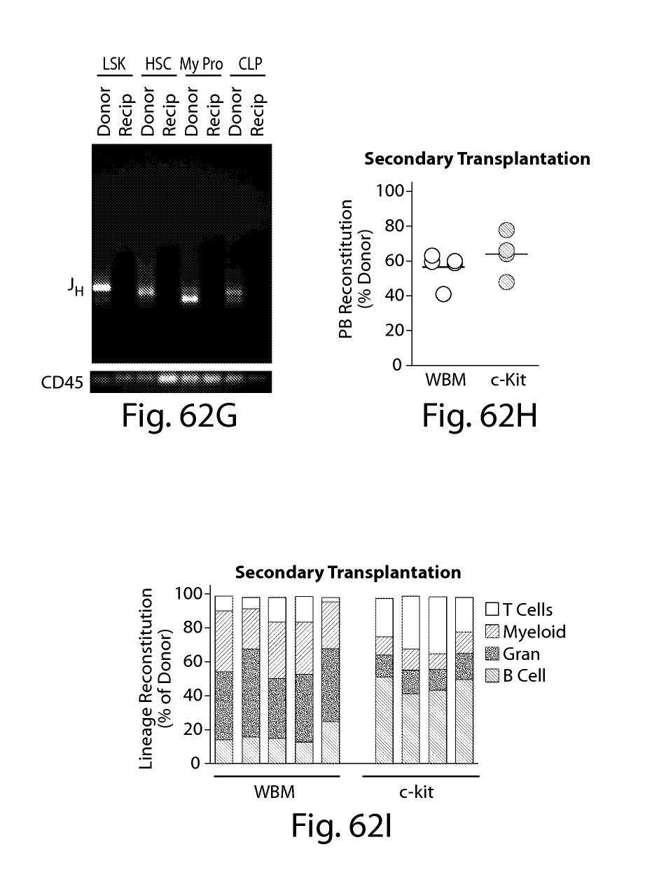

In addition to reconstituting the peripheral blood, HSCs efficiently engraft secondary hematopoietic organs and bone marrow progenitor cell compartments upon transplantation. To determine if the B-cell progenitors transduced with the 8-TF or 8-TFPoly cocktails possessed this ability, reconstituted mice were sacrificed and analyzed 18-20 weeks post-transplant, which showed that all the mice had donor-derived chimerism of the bone marrow, spleen and thymus though the level of varied between recipients as we had observed in the periphery (FIG. 62A). The Pro/Pre B-cell origin of the engrafting cells was confirmed through analysis of IG rearrangement from DNA isolated from granulocytes and myeloid cells purified from the bone marrow and spleen, and T-cells derived from the thymus (FIG. 62B) Immunophenotyping of bone marrow cells revealed donor contribution to common lymphoid progenitors (CLPs: lin−Flk2+IL7R□+ckitlowSca1low), CMPs, granulocyte/monocyte progenitors (GMPs: lin−ckit+Sca1−Fc□rhighCD34+), megarkaryocyte/erythrocyte progenitors (MEPs: lin−ckit+Sca1−Fc□r−CD34−), and primitive LSK progenitors (lin−Sca1+ckit+) (FIGS. 62C-F). Importantly, we also observed donor contribution to megakaryocyte progenitors (MkPs: lin−c-kit+Sca1−CD41+), and erythroid progenitors (EPs: lin−ckit+Sca1−Endoglin+) suggesting that the reconstituting cells were able of give rise to precursor cells of platelets and erythrocytes, lineages which we could not track in the peripheral blood in the congenic CD45-based transplantation system we used. Subfractionation of the LSK compartment revealed donor-derived reconstitution of the multi-potent progenitor (MPP1: lin−ckit+Sca1+CD34+Flk2−, MPP2: lin−c-kit+Sca1+CD34+Flk2+) and HSC (lin−c-kit+Sca1+CD34−Flk2−) compartments (FIGS. 62C-62F). Donor-marked progenitors and HSCs were found to be heavy chain rearranged confirming their B-cell origin (FIG. 62G).

A defining property of HSCs is their ability to self-renewal, a potential that can be evidenced by an ability to reconstitute secondary recipients upon serial transplantation. To test if the cells generated in our experiments possessed this potential we sacrificed primary recipient mice 18 weeks post-transplant and transplanted whole bone marrow or donor-derived c-kit+ cells into irradiated secondary congenic recipients. Peripheral blood analysis at 4, 8 and 12 weeks post-transplant reveled robust donor reconstitution of B-, T- and myeloid cells in all secondary recipient mice (FIGS. 62H-I). Taken together, these results indicate that transient ectopic expression of 8 transcription factors imparts multi-lineage reconstituting potential, reconstitutes bone marrow progenitor compartments, and enables long-term self-renewal potential—the functional hallmarks of HSCs—onto lineage-restricted B-cell progenitors.

### Reprogramming Terminally Differentiated Myeloid Cells to Transplantable HSC-Like Cells.

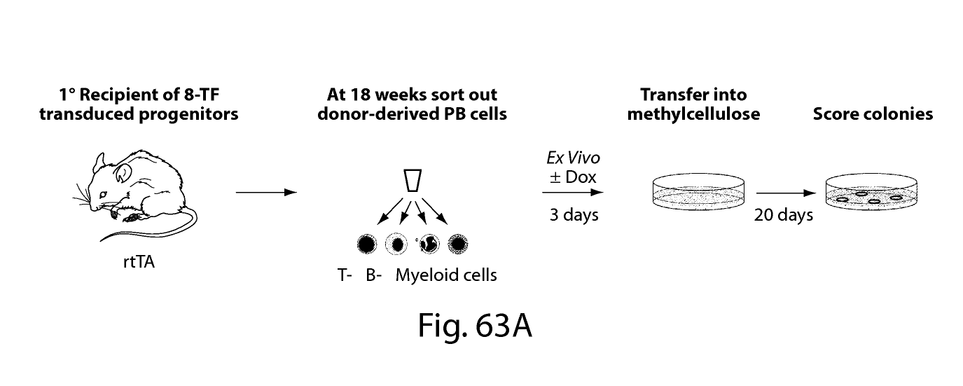

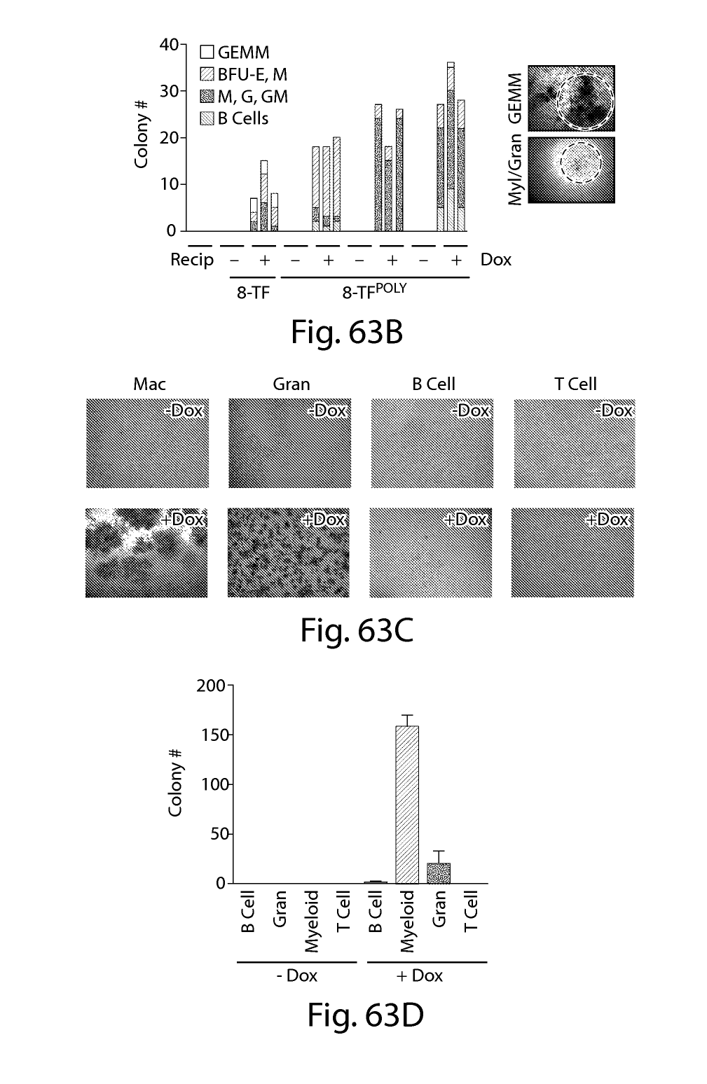

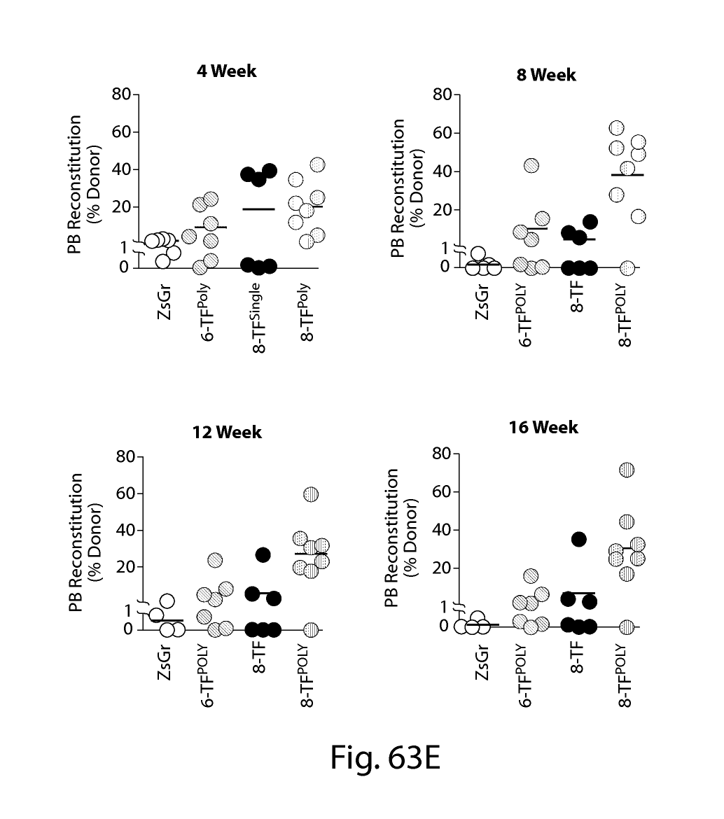

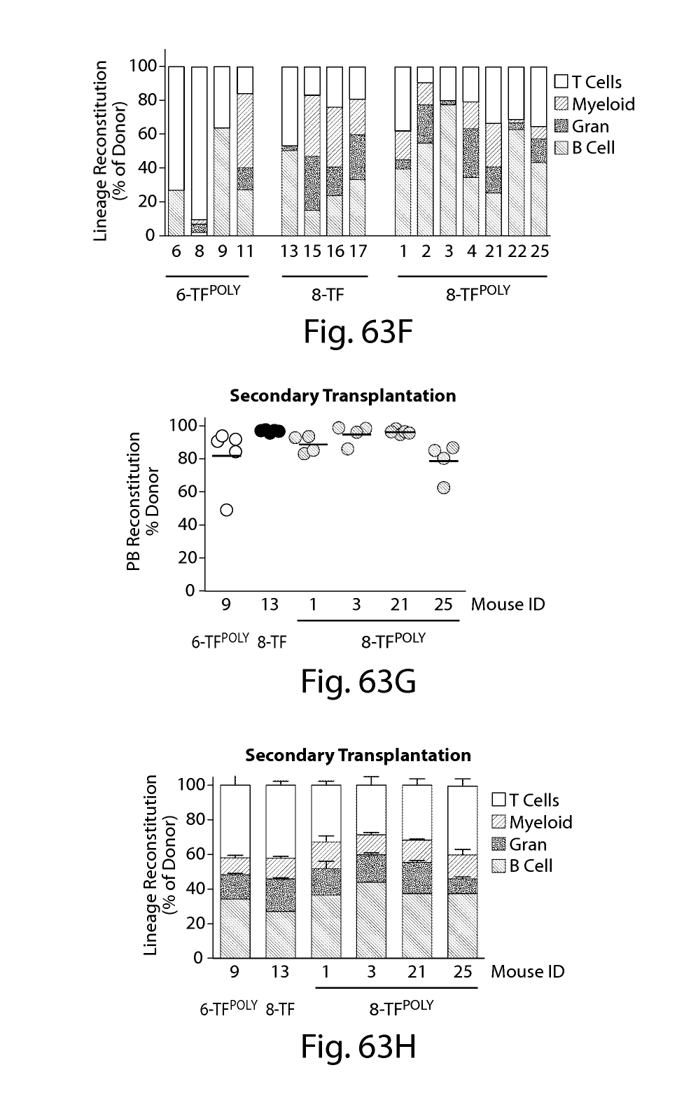

Eventual clinical translation of blood cell reprogramming to derive HSCs would likely benefit from an ability to reprogram cell types that can be readily and non-invasively obtained from the peripheral blood. We therefore sought to determine if multi-lineage progenitor activity could be conferred onto terminally differentiated blood cells using the transcription factors we identified. Recipient and donor-derived peripheral blood was sorted from mice engrafted with Pro/Pre B-cells transduced with the 8-factor cocktail (8-TF or 8TFPoly) 16-22 weeks post-transplant (ie. 14-20 weeks post-doxycycline induction). Sorted cells were then cultured in the absence or presence of doxycycline—with the latter condition intended to lead to re-expression of the transduced factors—followed by plating the cells in methylcellulose (FIG. 63A). As expected, neither the recipient-marked cells, nor the donor-derived cells cultured and plated in the absence of doxycycline gave rise to colonies, consistent with low-level progenitor activity in the peripheral blood of mice (FIG. 63B). By contrast, plates seeded with donor cells that had seen reactivation of the 8 transcription factors by exposure to doxycycline gave rise to mixed myeloid lineage colonies that included primitive GEMM colonies (FIG. 63B). To determine which lineage(s) in the peripheral blood had the potential to give rise to these colonies upon re-expression of the transcription factors, we sorted donor-derived B-cells, T-cells, myeloid cells and granulocytes from the 8-TF reconstituted mice, and tested their colony forming potential following culturing and plating in the absence or presence of doxycycline. These experiments revealed that essentially all colony-forming potential originated from the myeloid and granulocyte cell fractions (FIG. 63C-63D). Interestingly, the colonies generated from the sorted myeloid cells were much larger than those derived from granulocytes though a greater number of colonies arose from the latter.

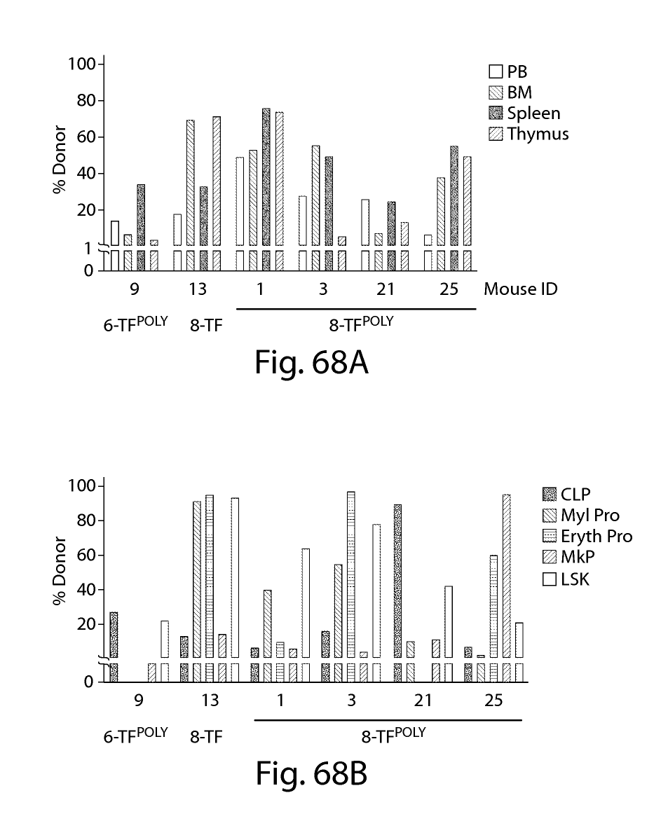

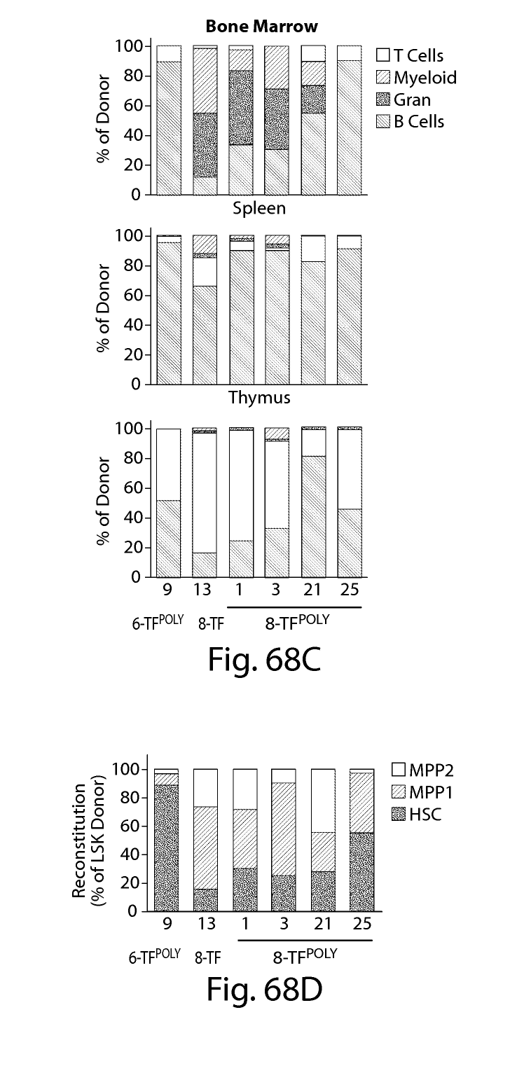

Encouraged by these results we next determined if the transcription factors we identified impart multi-lineage reconstituting potential onto terminally differentiated myeloid cells in transplantation assays. We sorted Mac1+c-kit− myeloid effector cells from Rosa26rtTA mice and transduced them with either 6-factor (6-TFPoly), or 8-factor cocktails (8-TF and 8-TFPoly) and transplanted them into irradiated congenic recipients. Peripheral blood analysis at monthly intervals revealed that, whereas none of mice transplanted with cells transduced with control virus were reconstituted, multiple recipients transplanted with cells transduced with 6-TFPoly (4/7), 8-TF (3/6), and 8-TFPoly (7/8) exhibited long-term donor-derived engraftment (FIG. 63F, 66). Lineage analysis of the reconstituted mice revealed donor-derived contribution to B-cell, T-cell, myeloid, and granulocyte lineages with the contribution to each lineage varying between recipients (FIG. 63F). Donor-derived contribution to secondary hematopoietic organs, and bone marrow progenitor cell compartments was observed in mice sacrificed and analyzed 20 weeks post-transplant (FIGS. 68A-D). Serial transplantation of donor-derived bone marrow cells demonstrated that the 6-TF or 8-TF transduced myeloid effectors could engraft secondary recipients in all lineages to 12 weeks post-transplant (FIG. 63G-63H).

Based on the functional data presented in FIGS. 58-63, we conclude that transient ectopic expression of 6 (Hlf, Runx1t1, Pbx1, Lmo2, Zfp37, and Prdm5) or 8 (Hlf, Runx1t1, Pbx1, Lmo2, and Zfp37, Prdm5, Mycn, and Meis1) transcription factors reprograms differentiated hematopoietic progenitors and effector cells to cells that possess the functional properties of HSCs. We term these reprogrammed cells induced-HSCs (iHSCs).

Single cell expression profiling of iHSCs reveals evidence of partial and full reprogramming.

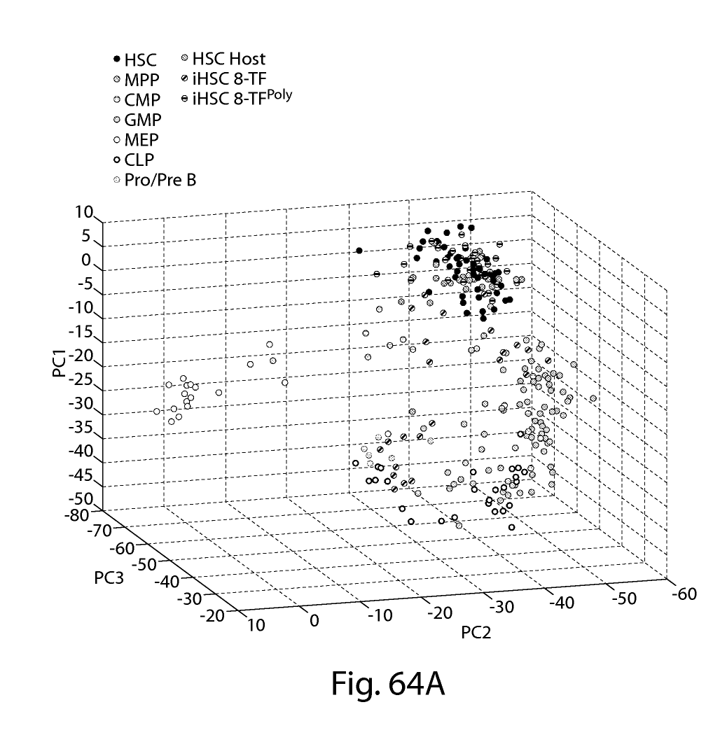

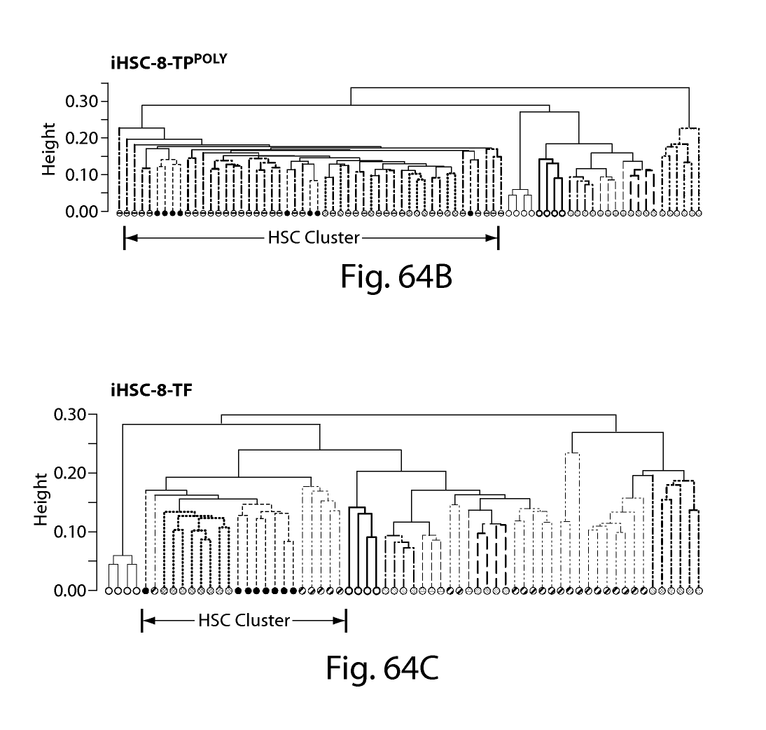

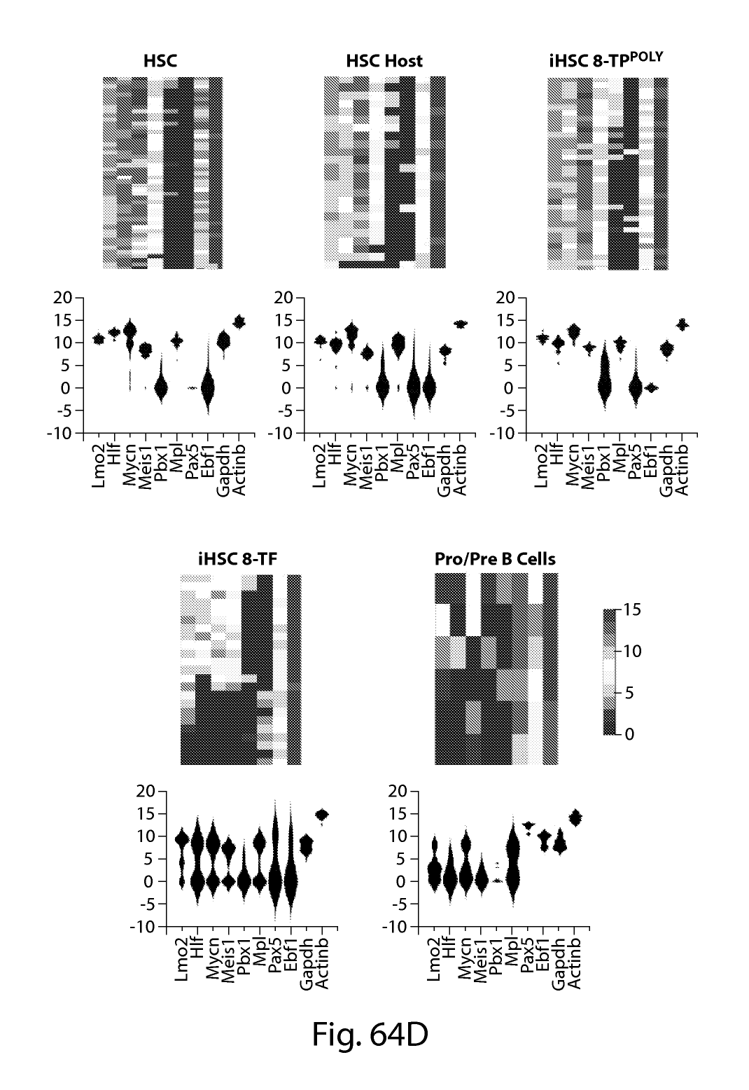

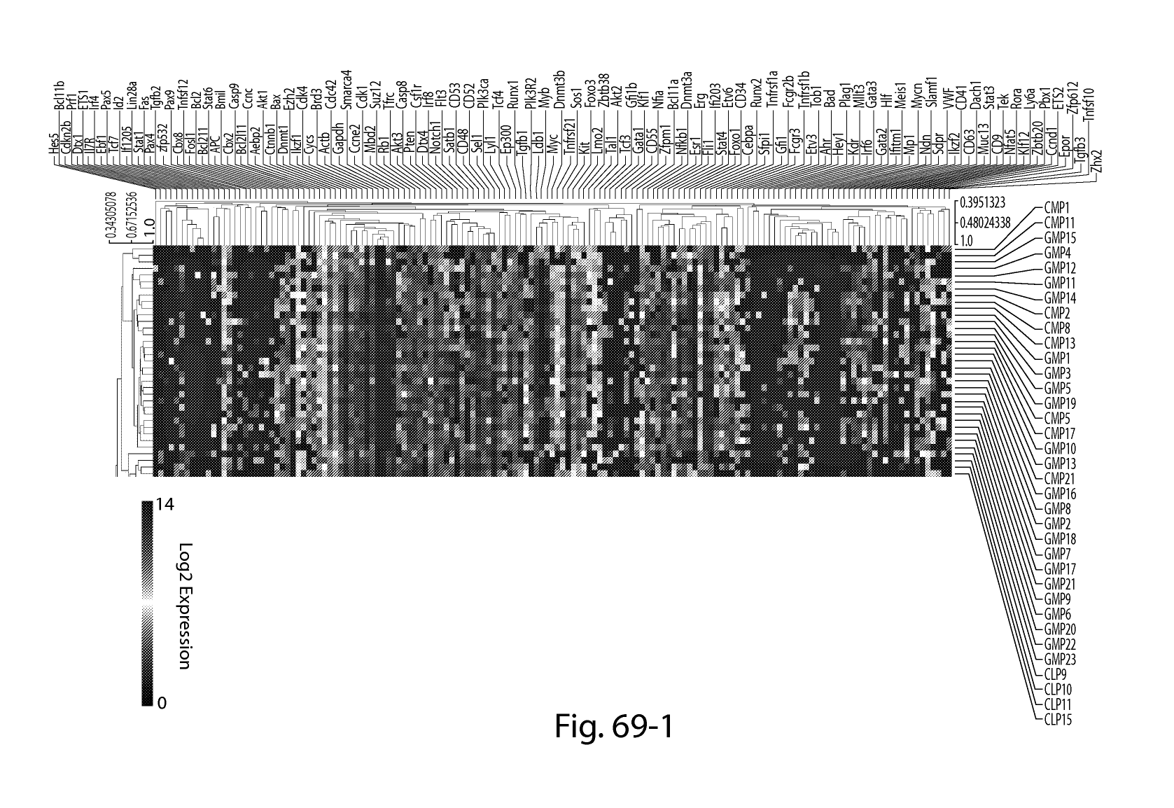

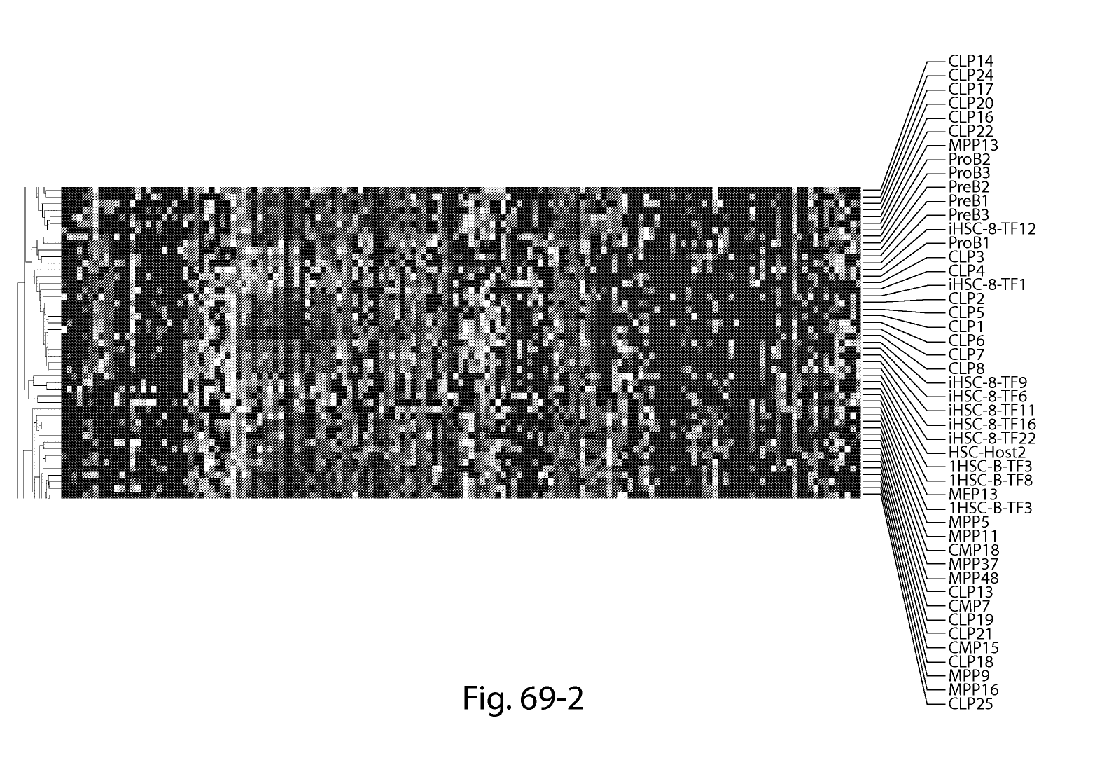

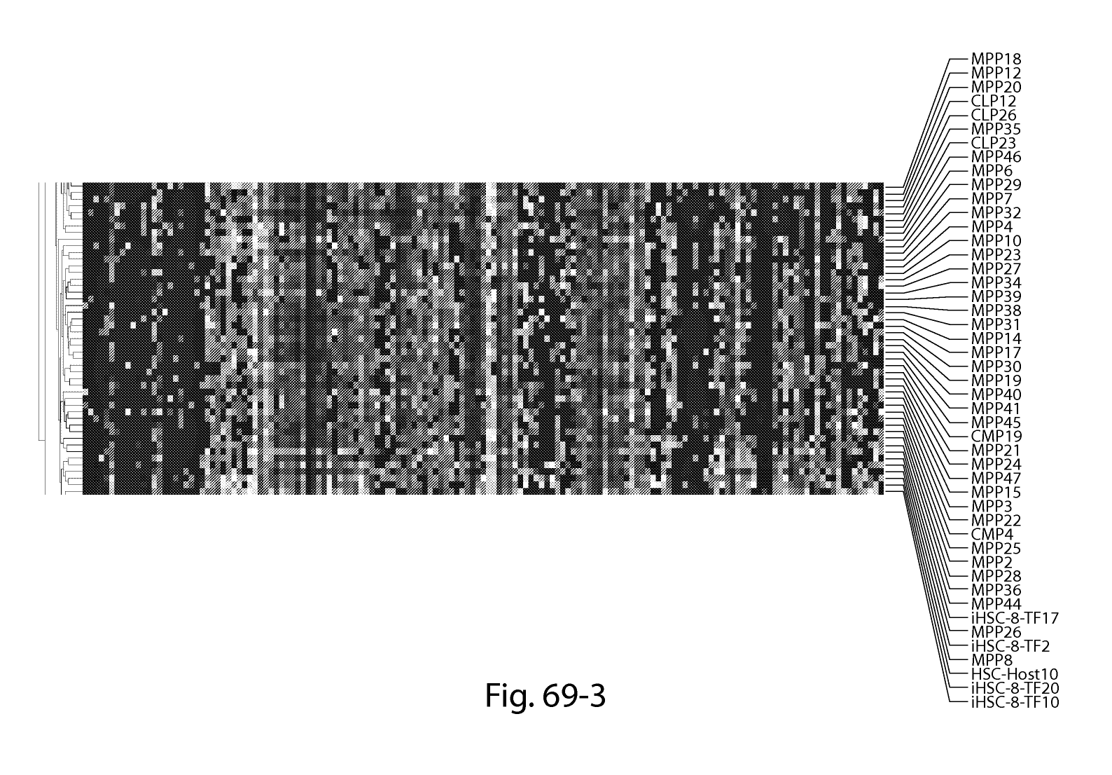

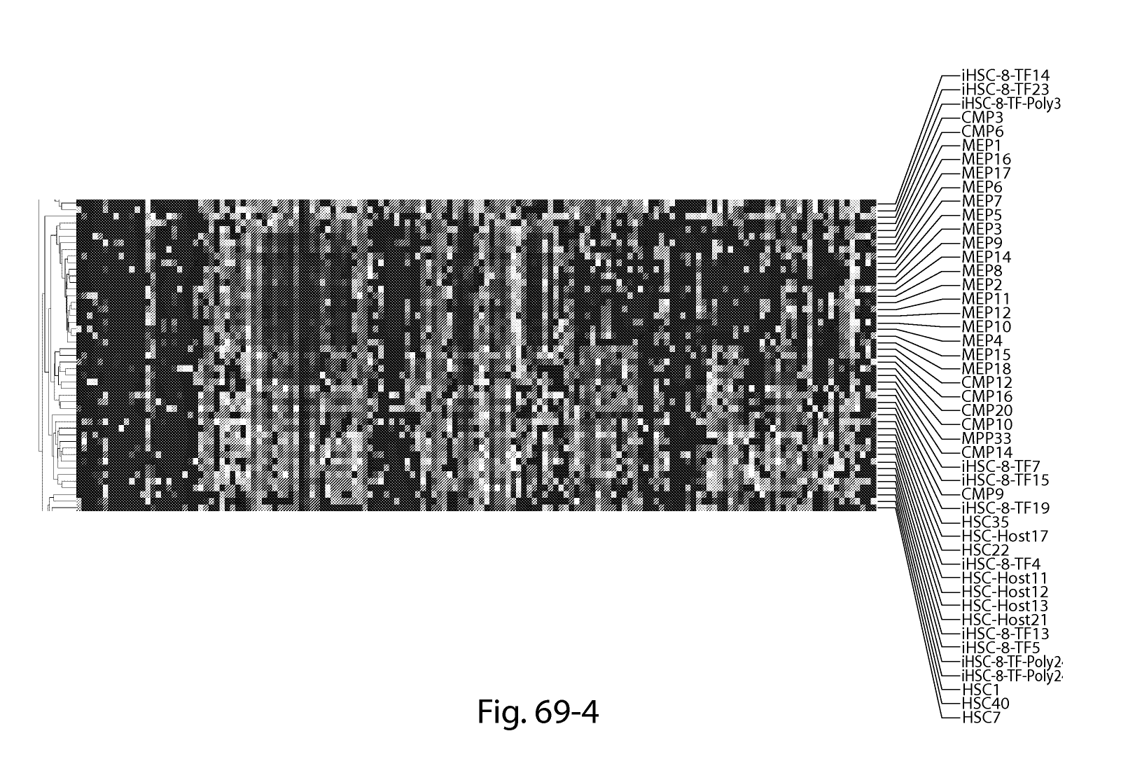

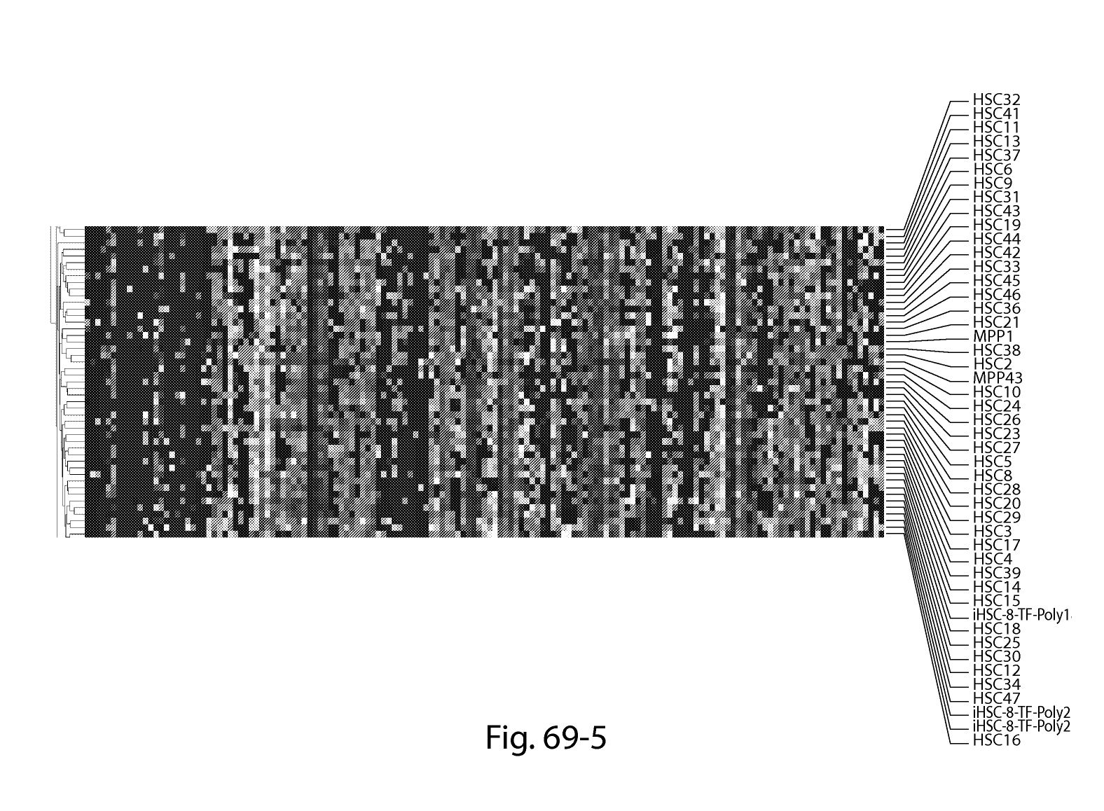

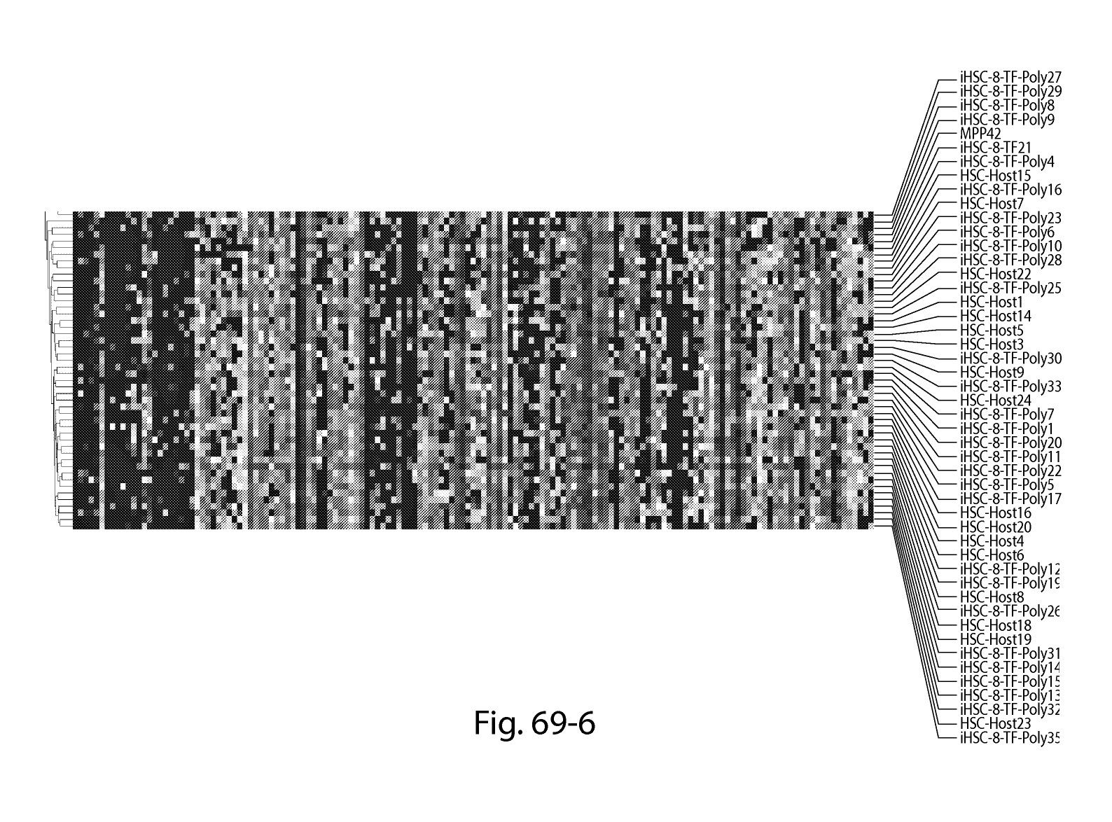

To assess the extent to which reprogrammed iHSCs recapitulate the molecular properties of endogenous HSCs, we employed a recently developed single cell gene expression profiling methodology that accurately defines hematopoietic stem and progenitor identity through the simultaneous quantification of expression of 152 lineage-specific transcription factors, epigenetic modifiers, cell surface molecules, and cell-cycle regulators (Guo et al., 2013). We sorted and analyzed donor-derived iHSCs by immunophenotype (CD45.2+lineage−ckit+Sca1+Fk2−CD34−/lowCD150+) from two different experiments in which Pro/Pre B cells had been transduced with the 8-TF cocktail as single viruses (8-TF), or with polycistronic viruses (8-TFPoly) (FIG. 61). In both settings mice exhibiting long-term multi-lineage donor-derived reconstitution were sacrificed at 18 weeks post-transplantation. We also sorted and analyzed host-derived HSCs (CD45.1+lineage−ckit+Sca1+Fk2−CD34−/lowCD150+) from the same mice to serve as controls. Single cell expression data generated from iHSCs and host HSCs was then analyzed in comparison to data generated from Pro/Pre B-cells (the starting cell type), and also to data previously generated from HSCs, MPPs, CLPs, CMPs, GMPs, and MEPs purified at steady-state (Guo et al., 2013). Analysis of the raw data revealed high correlation between gene expression for the vast majority of the control and test cell types (FIG. 69, Tables 6-8). To further interrogate the transcriptional relationships amongst all the cell types analyzed, we performed principal component analysis (PCA) to define the transcriptional distances between the cells. As expected, steady-state HSCs and progenitor cells were largely positioned in agreement with established lineal relationships where HSCs forming a clearly defined cluster, with MPPs positioned proximal, and oligopotent progenitors (MEPs, GMPs, CLPs) positioned more distal to HSCs (FIG. 64A). Pro/Pre B-cells positioned closely to CLPs consistent with the lineal relationship between these cell types, while the host-derived HSCs were positioned within the steady-state HSC cluster as expected (FIG. 64A). Interestingly, iHSCs derived from the two experiments (8-TF or 8-TFPoly) exhibited very distinct patterns of expression with the iHSCs derived from the 8-TF single virus experiment being more heterogeneous than the iHSCs derived from the 8-TFPoly transduced cells (FIGS. 64A, 69, Tables 6-8). As a result, PCA analysis of these cells showed that whereas some of the iHSCs 8-TF positioned closely or within the HSC cluster, others mapped closer to MPPs while others yet positioned closely to the Pro/Pre B cluster (FIG. 64A). By contrast, all of the iHSCs derived using the polycistronic viruses (iHSC 8-TFPoly) homogenously clustered within the HSC node (FIG. 64A). Unsupervised hierarchical clustering analysis confirmed that whereas approximately equal numbers of iHSCs derived using single viruses mapped closely to HSCs (7/23), others mapped closely to MPPs (7/23), while the remainder mapped more closely to Pro/Pre B cells (10/23) (FIG. 64B). In contrast, all of the iHSCs derived using the polycistronic approach showed very high similarity to host and control HSCs (35/35).

The inclusion of five (Mycn, Hlf, Lmo2, Meis1 and Pbx1) of the eight reprogramming factors amongst the 152 genes analyzed in these experiments allowed us to address how endogenous levels of these factors was reestablished in iHSCs post-reprogramming Consistent with their known roles in regulating HSCs, high levels of each of MycN, Hlf, Lmo2, and Meis1 were observed in steady-state HSCs, which contrasted the low levels observed in Pro/Pre B cells (FIG. 64D). Pbx1 expression was lower in the majority of HSCs and absent in Pro/Pre B cells. Conversely, Ebf1 and Pax5, which are critical transcription factors for B-cell development were expressed at high levels in Pro/Pre B cells and negligible levels in HSCs. Analysis of the expression of these genes in iHSCs again revealed distinct differences depending upon whether or not single or polycistronic viruses were used for their derivation. Whereas high levels of endogenous MycN, Hlf, Lmo2, Meis1 and moderate levels of Pbx1 was reestablished in many of the iHSCs derived using single viruses, low levels of these genes and high levels of Ebf1 and Pax5 were still observed in a significant fraction of the cells (FIG. 64D). By contrast, the expression of each of these genes in iHSCs derived using the polycistronic viruses fully recapitulated the expression patterns observed in the control HSCs (FIG. 64D), as was the expression of all other genes analyzed known to be critical for HSCs function including the transcription factors Gfi1b, Gata2, and Ndn, and the cytokine receptors Mpl, and c-kit (FIG. 64D, Tables 6-8). Taken together, these results demonstrate that 8-TF reprogramming of Pro/Pre B using single viruses generates iHSCs with transcriptional properties consistent with either full or partial reprogramming, whereas iHSCs derived under optimal polycistronic viral conditions exhibit an expression profile synonymous with HSCs.

### Discussion

Within the hematopoietic system, HSCs are the only cells with the functional capacity to differentiate to all blood lineages, and to self-renew for life. These properties, in combination with the ability of HSCs to engraft conditioned recipients upon transplantation, have established the paradigm for stem cell use in regenerative medicine. Allogeneic and autologous HSC transplantation is used in the treatment of ˜50,000 patients/year for congenital and acquired hematopoietic diseases and other malignancies (Gratwohl et al., 2010). Current challenges to transplantation therapies include the availability of histocompatible donor cells and associated graft versus host disease. De novo generation of isogenic HSCs from patient derived cells would obviate these issues, and extend transplantation to all patients as opposed to those for whom a histocompatible donor can be identified. Deriving HSCs from alternative cell types has thus has been a long sought after goal in regenerative medicine. Here we report the generation of induced-HSCs via reprogramming from committed hematopoietic progenitor and effector cells. Through identification and functional screening of 36 HSC-enriched factors, we identified 6 transcription factors Hlf, Runx1t1, Pbx1, Lmo2, Zfp37, and Prdm5 whose transient ectopic expression was sufficient to impart HSC functional potential onto committed blood cells. Inclusion of two additional transcription factors, Mycn, and Meis1, and the use of polycistronic viruses increased reprogramming efficacy. These findings demonstrate that ectopic expression of a small number of defined transcription factors in committed blood cells is sufficient to activate the gene regulatory networks governing HSC functional identity. The derivation of iHSCs therefore represents a novel cell-based system for exploring the mechanisms underlying the establishment and maintenance of fundamental HSC properties such as self-renewal and multi-lineage differentiation potential. Moreover, our results demonstrate that blood cell reprogramming is a viable strategy for the derivation of transplantable stem cells that could serve as a paradigm for eventual clinical application.

Despite the fact that HSCs are the most well characterized tissue-specific stem cells, surprisingly little is known about the molecular mechanisms involved in regulating their central properties. The identification of a defined set of transcription factors capable of stably imparting self-renewal and multi-lineage differentiation potential onto otherwise non-self-renewing, lineage-restricted cells, demonstrates that these factors are critically involved in regulating the transcriptional networks underlying HSC functional identity. Consistent with this, several of the factors that we identified have previously been shown to be important for regulating diverse aspects of HSC biology. For example, PBX1 and MEIS1, which interact and can form heterodimeric and heterotrimeric complexes with HOX proteins, have both been shown to regulate HSC self-renewal by maintaining HSC quiescence (Ficara et al., 2008; Kocabas et al., 2012; Unnisa et al., 2012). LMO2 is required for hematopoiesis and in its absence, neither primitive or definitive blood cells form (Warren et al., 1994; Yamada et al., 1998). And while MYCN is dispensable for HSC activity due to the functional redundancy of MYC, combined ablation of both Myc and MycN severely disrupts HSC self-renewal and differentiation potential (Laurenti et al., 2008). In contrast to these well-characterized genes, Prdm5 and Zfp37 remain unstudied in HSC biology, and though the role of RUNX1T1 (as known as ETO) as a fusion partner with RUNX1 in acute myeloid leukemia is well established, its role in normal hematopoiesis remains unclear. Defining the roles that each of the reprogramming factors play in normal HSC biology will be critical for understanding their function in blood cell reprogramming.

Going forward it will also be important to elucidate how the reprogramming factors activate and maintain the transcriptional networks underlying HSC functional identity in other cell types during reprogramming Given that 6 of the 8 factors we identified, Hlf (Inaba et al., 1992), Meis1 (Moskow et al., 1995), Lmo2 (Boehm et al., 1991), Mycn (Brodeur et al., 1984; Marx, 1984), Pbx1 (Kamps et al., 1991), and Runx1t1 (Erickson et al., 1992) are proto-oncogenes, suggests that blood cell reprogramming to iHSC likely involves the activation and/or repression of gene networks that are common to stem cells and transformed cells. This is also consistent with the finding that virtually all the transcription factors required for HSC formation, maintenance, or lineage commitment are targeted by somatic mutation or translocation in heme malignancy {Orkin, 2008 #5327}. Some insights into how the individual reprogramming factors mediate their activity has been provided by recent studies. For example, LMO2 overexpression in committed T-cell progenitors led to a preleukemic state characterized by sustained self-renewal activity yet without blocking T-cell differentiation potential, and this was associated with upregulation of a cadre of genes normally expressed by primitive hematopoietic stem and progenitor cells (HSPCs) (McCormack et al., 2010). Similarly, ectopic expression of HLF in downstream multi-potent and oligo-potent myeloid progenitors imbued them with potent self-renewal activity ex vivo without blocking their differentiation potential, which was associated with expression of CD150, and sustained repression of lineage commitment markers, phenotypes consistent with HSCs (Gazit et al.). HLF expression alone was nonetheless insufficient to impart HSC transplantation potential onto downstream progenitors (RG, BG, DJR unpublished). These studies show that while ectopic expression of HLF or LMO2 can instill at least some of the functional and molecular properties of HSCs onto committed blood cells, alone they cannot access the full repertoire of transcriptional programs needed to establish and maintain HSC function. In these regards, it is interesting that whereas iHSCs generated using polycistronic viruses all exhibited expression profiles that were indistinguishable from control HSCs, iHSCs generated using monocistronic viruses were heterogeneous at the molecular level with many of the cells analyzed showing clear evidence of partial reprogramming That some of these partially reprogrammed cells clustered closely to the Pro/Pre B cells from which they were derived suggests that these cells retained an epigenetic memory of their cell of origin despite being purified by an immunophenotype consistent with HSCs. It is likely that the partially reprogrammed iHSCs in the 8-TF single virus experiments did not receive the full complement of reprogramming factors. If so, further study of fully reprogrammed versus partially reprogrammed cells may provide mechanistic insights into how the reprogramming factors collaborate to activate the gene regulatory networks underlying HSC functional identity.

Although the transcriptional properties of iHSCs derived under optimal 8-TF polycistronic conditions were indistinguishable from endogenous HSCs, further analysis will be required to determine if the epigenetic landscape of these cells is fully reset to that of HSCs. In this regard, it was interesting that the lineage potential observed in our experiments in mice reconstituted with iHSCs sometimes, though not always, evolved over time post-transplantation, with donor-derived chimerism showing lineage skewing at early time points post-transplant, and more balanced output at later time points. These results suggest that iHSCs may need time to fully reset their epigenetic landscape to achieve balanced HSC potential, in a manner similar to the erasure of epigenetic memory observed with continued passage of iPS cells (Polo et al., 2010). Whether or not cell passage influences epigenetic resetting during iHSC derivation is at this point unclear. It is plausible that iHSCs may require a period of “maturation” in the stem cell niche to achieve full HSC potential. It is notable that some of the partially reprogrammed iHSCs we analyzed had not appropriately upregulated the MPL or KIT receptors suggesting an inability to transduce signals in response to TPO or SCF emanating from the niche.

Transcription factors play a critical role in the specification of different lineages during development, and as such the discovery of a set of transcription factors capable of activating the gene regulatory networks underlying HSC functional identity suggests that it may be possible to use these factors on cells derived from pluripotent stem cells to facilitate the generation of definitive HSCs. Along these lines, a recent study showed that expression of 5 transcription factors HOXA9, RORA, ERG, SOX4, and MYB was able to impart transient myeloerythroid engraftment potential onto iPS-derived blood cell progenitors, though these factors were unable to instill HSC potential onto the cells (Doulatov et al., 2013). It will also be important to test if the reprogramming factors we identified can be used to convert cell types outside of the hematopoietic system to an iHSC fate in a manner similar to the ability of the Yamanaka factors to bestow pluripotency onto cells of diverse lineages, though it remains possible that iHSCs derivation using the factors we defined will be limited to the blood system. Nonetheless, the generation of iHSCs via blood cell reprogramming represents a powerful new experimental paradigm for studying the fundamental mechanisms underlying HSC identity that might eventually be lead to the derivation of transplantable stem cells with clinical potential.

### Materials and Methods

Microarray: Microarray data was generated on the Affymetrix 430 2.0 platform and included previously published data generated in our lab in addition to datasets that were curated from GEO. Overall the database consists of 142 expression profiles from 40 FACs purified hematopoietic cell populations based on known cell surface phenotypes. All datasets were subjected to quality control (QC) measures provided in the ArrayQualityMetrics package of R/Bioconductor (http://www.bioconductor.org). Datasets were normalized (gcRMA) using R bioconductor. To identify potential regulators of HSCs, we applied a filter in which the ratio of expression in HSCs to all others had to be greater than 2.5-fold. The list of potential regulators was finalized by cross-referencing the literature to identify factors with known transcriptional/translation regulatory roles.

Mice: B6.SJL-Ptprca/BoyAiTac1 (Taconic Farms; Hudson, N.Y.) and C57BL/6N (Charles River Laboratories; Cambridge, Mass.) recipient mice and B6.CgGt(ROSA)26Sortm1(rtTA*M2)Jae/J donor mice (Jackson, Bar Harbor, Me.) were used. For some experiments, B6.CgGt(ROSA)26Sortm1(rtTA*M2)Jae/J mice crossed to the CD45.1 background were used. All mice were maintained according to protocols approved by Harvard Medical School Animal Facility and all procedures were performed with consent from the local ethics committees.

Pro/pre B-cell, CMP and HSC purification: Antibodies used in FACs purification included: CD34, Sca1, c-kit, AA4.1 from eBioscience (San Diego, Calif.); Fc□R from BD Bioscience (San Jose, Calif.); IgM Sigma Aldrich (St. Louis, Mo.); IL-7R□, Ter119, CD45.1, CD45.2, Mac1, CD3, CD4, CD8, Gr1, CD150, CD19, CD25 and B220 from BioLegend (San Diego, Calif.). 6-12 week old B6 CD45.2+rtTA heterozygous mice were sacrificed and the bone marrow harvested as previously described (Rossi et al. PNAS 2005). To obtain Pro/Pre B cells, a B220 enrichment was performed using biotin B220 (BD Bioscience), streptavidin magnetic beads and a magnetic column (Milteny Biotec). Enrichment was performed according to published protocols. To obtain CMPs, a c-kit enrichment using directly conjugated magnetic beads (BD Bioscience) was performed on whole bone marrow cells. Cells were sorted directly into sample media containing 2% FBS. All cells were sorted on a FACS Aria II (Becton Dickinson).

Virus Production: Factors were cloned into the pHage2 dox inducible system under the TRE reporter using restriction site directional (Not1 and BamH1) cloning as previously described (Gazit et al. 2013). Importantly, a number of these constructs were cloned out of a cDNA library created from FACS sorted HSCs. All constructs were checked by restriction diagnostics and fully sequenced. Constructs (FIG. 58B) include an IRES that enables ZsGr reporter expression. Polycistronics (FIG. 61A) combined individual viruses to create RHL and PZP. Individual factors (RUNX1T1, HLF and LMO2) and (PBX1, ZFP37 and PRDM5) were linked using non directional cloning and stepwise insertion into the respective restriction sites Sal1, Spe1, BamH1 separated by 2A sequences. All constructs were checked by restriction digest diagnostics and sequenced. Viruses for all the 36 factors were produced according to a previously established protocol (Mostoslaysky et al., 2005). All viruses are titered on Jurkat cells to an approximated working MOI ˜5.0.

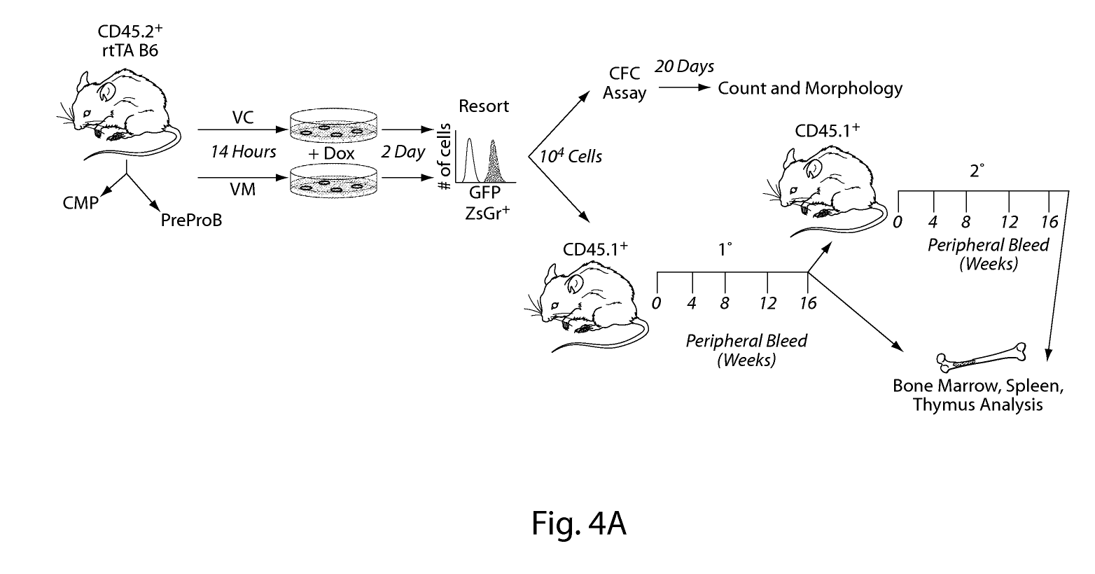

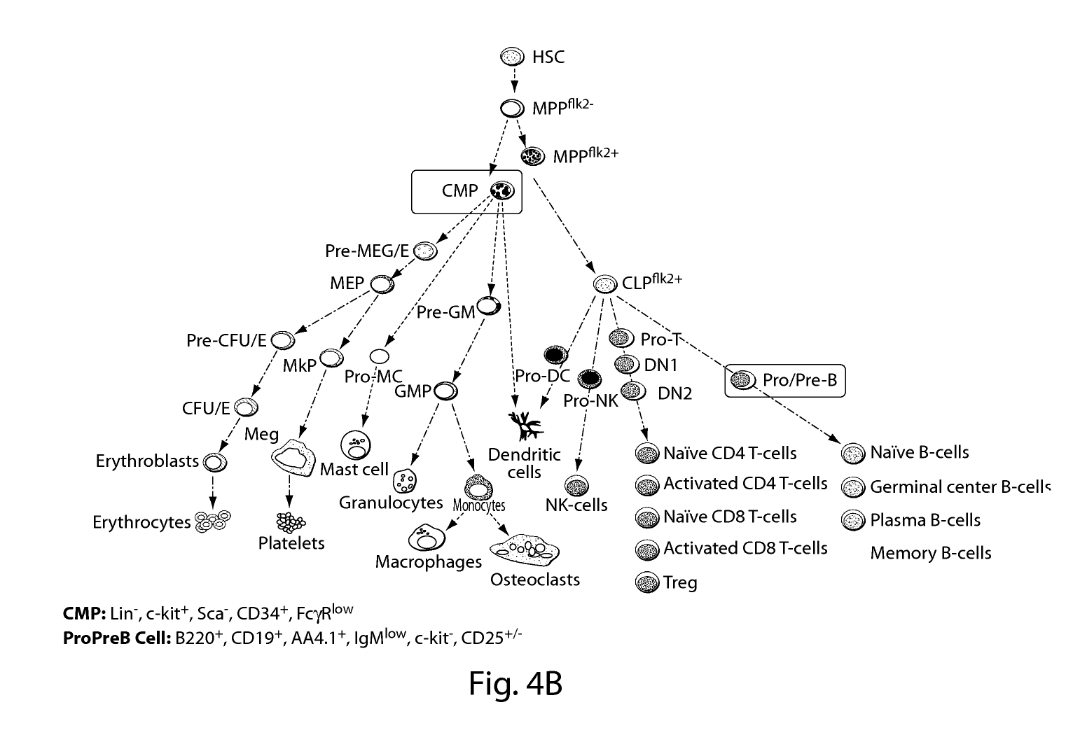

Pro/PreB and CMP CFC assays: Sorted Pro/Pre B cells and CMPs were isolated from rtTA transgenic CD45.2+ and when indicated CD45.1+ donors. 60,000 cells/200 uL media are incubated with the indicated viruses for 16 hours. Media used is Sclone supplemented with 10 ng/mL SCF, 10 ng/ml IL-12, 10 ng/ml TPO, 5 ng/mL Flk-3, and 5 ng/mL IL-7. After transduction, 1.0 mg/ml Doxacycline is added for 48 hours and then transferred to methylcellulose or transplanted. In the case of FIGS. 4-6, a 24 hour ex vivo dox induction was implemented because more cells appeared viable at this time point.

In CFC assays, 10,000 Pro/PreB or 1,000 CMP cells were transferred from the dox containing media to be diluted and mixed with 1.75 mL per well of M3630 methylcellulose (Stem Cell Technology) and plated into a 6 well dish. 20 days later the colonies were counted and characterized by morphology.

CFC secondary reprogramming ex vivo was accomplished by plating 60,000 donor-derived FACS sorted cells into a 12 well plate with 500 uL of F12 media supplemented with 10 ng/mL SCF, 10 ng/ml IL-12, 10 ng/ml TPO, 5 ng/mL Flk-3, and 5 ng/mL IL-7. When indicated 1.0 mg/ml dox was added for 72 hours. 10,000 cells were then directly transferred to 1.0 mL of methylcellulose in a 12 well format. 20 days later colonies were counted and characterized by classically defined morphologies.

Pro/Pre B cell Transplantation: Transplants were performed by combining 10,000 ZsGr+ resorted cells or 2.0×10⁶ unsorted Pro/Pre B/CMP cells with 2×105 B6 CD45.1+ competitor cells and transplanted intravenously into IR B6 CD45.1+ recipients. Alternatively, sorted and transduced Pro/Pre B cells and CMPs were injected non competitively with 2×10⁵ Sca1 depleted bone marrow cells (depletion performed with the Macs magnetic depletion columns previously described according to manufactures instructions). Peripheral bleeds were performed at 4, 8, 12, and 16 weeks. Post 16 weeks, the same analysis as peripheral blood was performed on the bone marrow, spleen, and thymus.

Serial transplantation was performed by isolating bone marrow from primary mice with reconstitution from either CD45.1+ Pro/Pre B cells (>1.0%) or CD45.2+ Mac1+ bone marrow cells (>5.0%). In the case of Pro/Pre B cells, whole bone marrow was counted and 107 cells were noncompetitively transplanted into CD45.2+ recipients. Alternatively (c-kit secondary), 10,000 FACS sorted doublet discriminated, live, lineage negative, c-kit+ donor CD45.1+ cells were transplanted non-competitively with 2×10⁵ Sca1 depleted cells into IR and conditioned recipients. Mac1+ bone marrow reconstituted whole bone marrow cells were FACS sorted on donor (CD45.2+). Generally, 5.0×10⁶ donor-derived FACs sorted cells were transplanted noncompetitively into conditioned and IR recipients. Peripheral bleeds were performed at 4, 8 and 12 weeks.

Peripheral Blood Analysis and Bone Marrow Analysis: Flk2, CD34, c-kit and Sca1 antibodies were purchased from eBioscience (San Diego, Calif.). FcgR3 (CD16) was purchased from BD Bioscience (San Jose, Calif.). IL-7R□, SLAM (CD150), Ter119, CD45.1, CD45.2, B220, Mac1, CD3, CD4, CD8, Gr1 (Ly-6G/Ly-6C) were purchased from Biolegend (San Diego, Calif.)

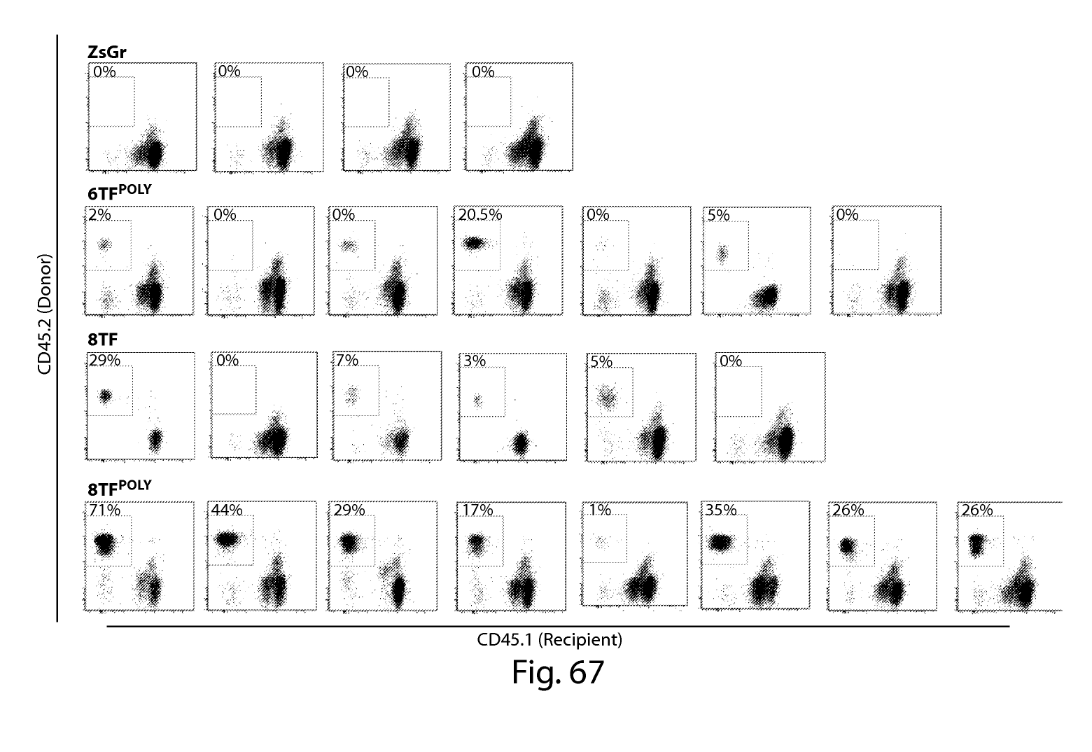

Staining for both the peripheral blood and the progenitor compartments was done as previously described (Beerman, Rossi, Bryder). Examples of cell stains and gating strategies are described for peripheral blood (FIGS. 59B, 60E, 61C and 63G) and bone marrow analysis (FIGS. 62A-62I and 67). In general, peripheral blood populations include: B cells (B220+), Myeloid cells (Mac1+ and Gr1−), Granulocyte (Mac1+ and Gr1+), T Cells (CD3+/CD4+/CD8+).

Progenitor populations are defined as such: All are doublet discriminated, live (PI negative) and lineage negative (Gr1−, Mac1−, B220−, CD3−, CD4−, CD8−, Ter119−−). Hematopoietic progenitors (HSC, MPP1, and MPP2) were gated c-kit+Sca1+ then defined by flk2 and CD34 expression. Common lymphoid progenitors (CLPs) were gated flk2+ IL-7R+ then defined by c-kit and Sca1 status. Myeloid Progenitors (GMP, CMP, and MEP) were gated c-kit+Sca1− and defined by Fc□R3 and CD34 expression. Erythroid progenitors (EP) and Megakaryocyte Precursors (MkP) were both gated c-kit+Sca1− but defined respectively by Endoglin and CD41 expression.

VDJ Rearrangement—Heavy and light chain (kappa and lambda) recombinational events were tested using a PCR based assay established by Brisco et al. (British Journal of Hematology 1990; 75:163-167) and Busslinger et al. (Nature 2007; 449:473-481). In overview, the strategy spans the region from VH2 to JH4, Therefore, covering the predominant recombinational events of heavy chain rearrangement. All PCR based strategies were confirmed on both bone marrow and peripheral blood positive and negative controls.

Transcription Factor Integration—To test for viral integration of the factor to be expressed primers were designed to generate products over intron-exon barriers (FIG. 59F). Endogenous products are eliminated by their larger size or that the primers will not extend over the intron. Rigorous controls were performed to ensure that false positives would not be detected. All primers proved negative when they singly were subtracted from the 36 factor mix and when ZsGr control virus is used, only when the factor is present does the band appear. Primers are listed in the Supplementary Table 1. PCR conditions were performed according to manufactures instructions (Kappa Biosystems).

High throughput single cell qPCR and computational analysis: Individual primer sets were pooled to a final concentration of 0.1 μM for each primer. Individual cells were sorted directly into 96 well PCR plates loaded with 5 μL RT-PCR master mix (2.5 μL CellsDirect reaction mix, Invitrogen; 0.5 μL primer pool; 0.1 μL RT/Taq enzyme, Invitrogen; 1.9 μL nuclease free water) in each well. Sorted plates were immediately frozen on dry ice. After brief centrifugation at 4° C., the plates were immediately placed on PCR machine. Cell lyses and sequence-specific reverse transcription were performed at 50° C. for 60 minutes. Then reverse transcriptase inactivation and Taq polymerase activation was achieved by heating to 95° C. for 3 min. Subsequently, in the same tube, cDNA went through 20 cycles of sequence-specific amplification by denaturing at 95° C. for 15 sec, annealing and elongation at 60° C. for 15 min After preamplification, PCR plates were stored at −80° C. to avoid evaporation. Pre-amplified products were diluted 5-fold prior to analysis. Amplified single cell samples were analyzed with Universal PCR Master Mix (Applied Biosystems), EvaGreen Binding Dye (Biotium) and individual qPCR primers using 96.96 Dynamic Arrays on a BioMark System (Fluidigm). Ct values were calculated using the BioMark Real-Time PCR Analysis software (Fluidigm).

Gene expression levels were estimated by subtracting the background level of 28 by the Ct level, which approximately represent the Log 2 gene expression levels. Principal component analysis (PCA) was performed in Matlab to project all the control and experimental cells onto a three dimensional space to aid visualization. An unsupervised hierarchical clustering was used to cluster representative control cells and all the iHSC 8-TF or iHSC 8-TFPoly cells. The analysis was done with R using the average linkage method and a correlation-based distance. The representative control cells were selected as those whose expression levels were closest to the median based on Euclidean distance. Eight HSC cells, eight HSC Host cells, all six Pro/Pre B-cells, and four from each of the remaining control cell types were selected. The dendrogram branches were color-coded by cell type, as in the PCA analysis. Violin plots and the correlation heatmaps were generated with Matlab. The master heatmap of all the raw data (Supplement to FIGS. 64A-64D) was generated with MultiExperiment Viewer (MeV) program (http://www.tm4.org/mev.html) using the default setting.

TABLE 6-1
Single cell expression data (reduced list)---Control

| Factor | HSC-Host1 | HSC-Host2 | HSC-Host3 | HSC-Host4 | HSC-Host5 | HSC-Host6 |
|---|---|---|---|---|---|---|
| Actb | 13.2775869 | 14.168841 | 13.9178852 | 14.0751018 | 14.3746391 | 14.7443427 |
| Aebp2 | 6.28419787 | 6.32255813 | 7.19444936 | 5.65953541 | 6.95783404 | 7.26360494 |
| Ahr | 0 | 7.57209355 | 0 | 0 | 0 | 0 |
| Akt1 | 9.4500759 | 0 | 10.0765631 | 9.94327921 | 10.6548673 | 10.0745346 |
| Akt2 | 6.22818312 | 0 | 6.70532413 | 0.8889789 | 6.47748177 | 5.95383663 |
| Akt3 | 7.51547845 | 0 | 6.07943514 | 6.17938762 | 6.4222982 | 7.17078745 |
| APC | 7.79584916 | 0 | 6.19688147 | 0 | 0 | 0 |
| Bad | 0 | 0 | 0 | 0 | 0 | 0 |
| Bax | 8.2648093 | 9.18808438 | 6.51775922 | 9.27759397 | 6.43362681 | 9.23990229 |
| Bel11a | 0 | 3.15885611 | 0 | 5.12533276 | 4.04738876 | 0 |
| Bcl11b | 0 | 0 | 0 | 0 | 0 | 0 |
| Bcl2 | 6.98611579 | 5.59253753 | 5.86437743 | 5.82350133 | 5.38565841 | 6.25071983 |
| Bcl2l1 | 6.3386176 | 7.46201946 | 5.95513383 | 7.54053745 | 8.78325414 | 9.89410694 |
| Bcl2l11 | 0 | 0 | 6.94600503 | 6.87358216 | 4.32552584 | 7.85341182 |
| Bmi1 | 6.84030124 | 7.45817288 | 8.3898639 | 8.30544124 | 8.55457965 | 9.47756119 |
| Brd3 | 7.90377097 | 0 | 7.95461448 | 5.59030834 | 9.00631299 | 9.052141 |
| Casp8 | 7.51030052 | 8.02616926 | 4.9493906 | 8.5494905 | 8.91073923 | 7.93953605 |
| Casp9 | 0 | 0 | 8.5609996 | 1.67117364 | 4.0331817 | 9.80298865 |
| Cbx2 | 2.56416415 | 5.63988167 | 5.00035293 | 0 | 7.4548439 | 5.99738299 |
| Cbx8 | 0 | 0 | 0 | 0 | 0 | 0 |
| Ccnc | 0 | 7.05018411 | 6.61535219 | 7.14719604 | 0 | 0 |
| Ccnd1 | 9.03626766 | 0 | 10.6728171 | 9.38229874 | 9.65405424 | 11.2577639 |
| Ccne2 | 6.17995523 | 0 | 0 | 7.11543157 | 3.58571536 | 6.20681303 |
| CD34 | 9.47324504 | 4.55399303 | 0 | 6.67982887 | 8.80998961 | 8.42129488 |
| CD41 | 6.83783924 | 0 | 0 | 7.46208028 | 5.97956704 | 7.65198306 |
| CD48 | 0 | 5.56947557 | 0 | 0 | 0 | 0 |
| CD52 | 3.35679477 | 11.0232754 | 4.14631098 | 2.71474755 | 0 | 0 |
| CD53 | 8.20861996 | 9.55294311 | 10.642603 | 0 | 10.0045947 | 8.2383003 |
| CD55 | 5.73982206 | 7.34724526 | 0 | 8.36090066 | 0 | 6.70252191 |
| CD63 | 7.99968851 | 3.87874565 | 8.90775134 | 6.61989086 | 7.62771038 | 8.83849433 |
| CD9 | 7.44138139 | 6.21616714 | 6.50446133 | 8.246429 | 7.64906334 | 8.63028596 |
| Cdc42 | 12.1710731 | 11.0591526 | 12.4549519 | 11.9800985 | 12.2018552 | 11.6731426 |
| Cdk1 | 0 | 0 | 0 | 6.25722026 | 8.10356032 | 0 |
| Cdk4 | 7.18574541 | 0 | 8.80614599 | 8.60901532 | 8.72742091 | 8.91034066 |
| Cdkn2b | 0 | 3.88923712 | 0 | 0 | 3.6614691 | 0 |
| Cebpa | 0 | 0 | 0 | 0 | 2.11474663 | 0 |
| Csf1r | 0 | 0 | 0 | 0 | 0 | 0 |
| Ctnnb1 | 6.77574215 | 5.35561197 | 8.53644908 | 6.17550579 | 8.17135019 | 8.90801971 |
| Cycs | 9.45352333 | 8.28562581 | 9.69867329 | 9.15788233 | 8.5747268 | 11.0355392 |
| Dach1 | 10.8615494 | 0 | 9.31769339 | 9.02821771 | 8.02501106 | 10.7915469 |
| Dnmt1 | 7.9760193 | 7.79001706 | 9.59934161 | 9.46537455 | 10.1834542 | 9.73235565 |
| Dnmt3a | 9.17213793 | 6.74216981 | 10.3864007 | 8.88588303 | 10.0903643 | 9.57095471 |
| Dnmt3b | 7.6743627 | 0 | 8.58221524 | 8.13192866 | 6.41659753 | 10.5256969 |
| Dtx1 | 0 | 3.41522411 | 2.46078468 | 0 | 0 | 0 |
| Dtx4 | 0 | 0 | 8.6835801 | 0 | 2.66840805 | 0 |
| Ebf1 | 0 | 6.662193 | 0 | 0 | 0 | 0 |
| Ep300 | 9.71487536 | 9.16729643 | 9.43974794 | 9.62406494 | 8.10311513 | 8.26149733 |
| Epor | 8.68447169 | 7.68763276 | 7.25429274 | 7.04722818 | 8.24346493 | 6.54478382 |
| Erg | 9.20284562 | 0 | 8.87410211 | 11.3197691 | 11.1784466 | 10.0567225 |
| Esr1 | 8.43503126 | 0 | 9.11129812 | 10.8937654 | 8.57545747 | 8.3892723 |
| ETS1 | 0 | 7.93156712 | 8.24336392 | 8.54381125 | 0 | 7.97895885 |
| ETS2 | 7.69340598 | 10.4359154 | 7.88475206 | 9.15565609 | 9.36749687 | 9.44827774 |
| Etv3 | 0 | 4.64796195 | 0 | 4.71186206 | 6.09191076 | 4.93626547 |
| Etv6 | 10.9918334 | 8.3432591 | 12.062043 | 10.4969697 | 11.0891387 | 10.5930954 |
| Ezh2 | 0 | 0 | 6.2199413 | 0 | 7.2175748 | 0 |
| Fas | 0 | 0 | 0 | 0 | 0 | 6.34199177 |
| Fcgr2b | 7.06819715 | 6.31957073 | 0 | 0 | 6.89220045 | 0 |
| Fcgr3 | 3.08395665 | 0 | 5.1508941 | 0 | 5.42301679 | 4.43817889 |
| Fli1 | 10.9830573 | 8.55863827 | 11.2140047 | 10.3178185 | 11.6619233 | 12.1483502 |
| Flt3 | 6.20637493 | 0 | 0 | 0 | 0 | 0 |
| Fosl1 | 4.69007508 | 0 | 0 | 0 | 0 | 0 |
| Foxo1 | 10.3454599 | 7.31474333 | 10.967598 | 9.8657691 | 10.5194737 | 10.0861124 |
| Foxo3 | 9.0799276 | 0 | 9.7189551 | 7.47165548 | 8.73488596 | 7.96186755 |
| Gapdh | 8.55078967 | 5.52545622 | 9.00242399 | 8.73312904 | 8.52812774 | 9.39231339 |
| Gata1 | 5.60159574 | 0.06279515 | 5.86590598 | 7.1439751 | 0 | 0 |
| Gata2 | 5.74244502 | 0 | 7.41208662 | 7.07920109 | 7.70789061 | 8.00674346 |
| Gata3 | 8.00418853 | 7.18159892 | 8.43773446 | 5.50080971 | 8.12295844 | 8.25560613 |
| Gfi1 | 0 | 6.18652121 | 10.6772443 | 7.54787108 | 7.83956553 | 0 |
| Gfi1b | 0 | 0 | 0 | 5.51370457 | 0 | 0 |
| Hes5 | 0 | 0 | 0 | 0 | 0 | 0 |
| Hey1 | 0 | 0 | 0 | 0 | 0 | 0 |
| Hlf | 10.44305 | 0 | 10.7025095 | 9.93038235 | 10.4823111 | 12.2258256 |
| Id2 | 5.87344248 | 0 | 7.19031139 | 5.96142885 | 0 | 6.51341399 |
| Ifi203 | 11.7852987 | 9.71801159 | 11.3716491 | 11.0104458 | 12.6373979 | 11.6777944 |
| Ifi205 | 4.67282232 | 0 | 7.24586334 | 0 | 0 | 0 |
| Ifitm1 | 12.1471017 | 4.92118909 | 13.4884472 | 12.662214 | 12.5615878 | 13.0294612 |
| Ikzf1 | 8.64469135 | 7.79726997 | 7.85685442 | 8.12528579 | 9.68635073 | 8.48962708 |
| Ikzf2 | 7.81120077 | 0 | 9.37252819 | 8.30677295 | 7.26836862 | 0 |
| I17R | 0 | 0 | 0 | 0 | 0 | 0 |
| Irf4 | 0 | 0 | 0 | 0 | 0 | 0 |
| Irf6 | 0 | 0 | 0 | 4.20551755 | 4.88594856 | 3.08177568 |
| Irf8 | 0 | 8.68822939 | 0 | 0 | 0 | 6.53060321 |
| Kdr | 0 | 0 | 0 | 0 | 0 | 0 |
| Kit | 11.2070686 | 0 | 11.6440993 | 11.4804292 | 12.2611324 | 12.206451 |
| Klf1 | 6.92350949 | 1.98980206 | 0 | 4.56789131 | 0.13589585 | 0 |
| Klf12 | 7.06267367 | 0 | 4.57402202 | 6.08382143 | 7.94374986 | 3.9594648 |
| Ldb1 | 10.4073068 | 7.3896168 | 10.1500409 | 10.0911962 | 10.7267532 | 11.0127515 |
| Lin28a | 7.17248465 | 0 | 5.58873198 | 6.56573609 | 6.38615843 | 3.82188034 |
| Lmo2 | 10.9902154 | 6.18088066 | 10.6616656 | 10.3550894 | 11.1327095 | 10.9913151 |
| Ly6a | 9.77053874 | 11.2332276 | 11.7270289 | 8.28647953 | 12.6717193 | 10.3350604 |
| Lyl1 | 0 | 2.97626088 | 1.79806679 | 7.18080529 | 6.9416814 | 6.73671636 |
| Mbd2 | 8.49739572 | 8.19189415 | 8.04081234 | 9.76536757 | 9.15455462 | 8.59064535 |
| Meis1 | 8.29093013 | 0 | 7.29725525 | 7.26528892 | 8.67247017 | 9.42229127 |
| Mllt3 | 5.89848994 | 0 | 0 | 6.69623752 | 4.4179384 | 3.79041107 |
| Mpl | 11.2861484 | 0 | 11.0645033 | 10.5099396 | 9.03000686 | 11.3155121 |
| Muc13 | 8.25899032 | 0 | 8.64152378 | 9.29492519 | 10.7390115 | 9.98391777 |
| Myb | 12.4569362 | 0 | 12.2263569 | 12.4668319 | 11.4934181 | 12.0411759 |
| Myc | 7.58661569 | 6.21232154 | 9.20695093 | 8.73071418 | 9.41854475 | 10.7856834 |
| Mycn | 12.9947643 | 0 | 13.0918794 | 13.9626228 | 12.9338862 | 12.4445334 |
| Ndn | 8.80844917 | 6.48582533 | 0 | 10.4252572 | 8.84853759 | 9.65347239 |
| Nfat5 | 10.4466948 | 9.45749742 | 10.690876 | 10.0164749 | 10.9448261 | 10.5579754 |
| Nfia | 9.61905092 | 0 | 7.82309617 | 10.1397415 | 0 | 10.3055652 |
| Nfkb1 | 0 | 0 | 0 | 0 | 0 | 2.96900953 |
| Notch1 | 0 | 0 | 9.29999671 | 0 | 7.33702794 | 0 |
| Pax4 | 0 | 0 | 0 | 3.25862559 | 0 | 0 |
| Pax5 | 0 | 8.92648494 | 0 | 0 | 0 | 0 |
| Pax9 | 2.08863054 | 0 | 0 | 0 | 0 | 5.05619592 |
| Pbx1 | 1.42391331 | 0 | 0 | 0 | 0 | 0 |
| PIk3ca | 8.96748889 | 6.64436068 | 9.27732513 | 8.90571616 | 7.62247587 | 8.4100092 |
| PIk3R2 | 9.65824684 | 0 | 9.22847732 | 7.39263343 | 0 | 4.40944775 |
| Plag1 | 0 | 0 | 7.01820576 | 7.02904616 | 3.5641265 | 0 |
| Prf1 | 0 | 0 | 0 | 0 | 1.57408799 | 0 |
| Pten | 10.9497819 | 0 | 10.2918594 | 8.92771496 | 10.4641876 | 10.3191806 |
| Rb1 | 8.96820297 | 10.0038452 | 9.14142412 | 9.85888737 | 8.18977625 | 9.89607842 |
| Rora | 5.35194121 | 4.24098601 | 5.85010593 | 4.61334456 | 5.97348017 | 8.17380426 |
| Runx1 | 0 | 7.58178739 | 8.9334852 | 0 | 7.0497458 | 0 |
| Runx2 | 4.95241455 | 0 | 0 | 0 | 5.41048102 | 5.81273837 |
| Satb1 | 0 | 0 | 0 | 0 | 7.86361531 | 0 |
| Sdpr | 0 | 0 | 0 | 0 | 2.58354882 | 3.26451236 |
| Sell | 0 | 0 | 2.34457587 | 0 | 0 | 0 |
| Sfpi1 | 9.71796118 | 7.47768178 | 8.88184673 | 7.30312418 | 8.77086956 | 10.3270219 |
| Slamf1 | 8.97990603 | 0 | 3.04564598 | 8.47261051 | 7.18152704 | 8.21009783 |
| Smarca4 | 10.4765281 | 3.61354971 | 10.1872564 | 10.8633232 | 8.60015526 | 10.9354338 |
| Sos1 | 4.33343207 | 0 | 3.63532361 | 0 | 5.53536226 | 6.14254392 |
| Stat1 | 3.23775129 | 0.21307953 | 7.58861399 | 3.02927896 | 8.80721388 | 3.51485392 |
| Stat3 | 10.6966168 | 7.76941207 | 10.6364369 | 10.0799192 | 10.5294486 | 11.2164717 |
| Stat4 | 9.20300453 | 0 | 7.8248698 | 9.2674567 | 8.94657563 | 9.64694998 |
| Stat6 | 9.03894911 | 8.52947719 | 9.97364377 | 9.05233066 | 9.64957237 | 11.0757572 |
| Suz12 | 6.16330105 | 5.48666925 | 9.32289767 | 8.71099601 | 7.89367605 | 8.06855486 |
| Tal1 | 8.36403791 | 0 | 2.33394532 | 0 | 1.38047772 | 0 |
| Tcf3 | 10.4218407 | 9.72305906 | 0 | 0 | 8.61448405 | 0 |
| Tcf4 | 9.16127496 | 9.85224012 | 11.534616 | 11.3598757 | 5.53155003 | 8.0963221 |
| Tcf7 | 0 | 0 | 0 | 0 | 0 | 1.57791407 |
| Tek | 0 | 0 | 0 | 7.32114021 | 0 | 6.95981526 |
| Tfrc | 9.28718925 | 7.02384574 | 0 | 8.22631353 | 0 | 9.43880717 |
| Tgfb1 | 5.88177291 | 0 | 0 | 0 | 0 | 0 |
| Tgfb2 | 0 | 0 | 0 | 0 | 0 | 0 |
| Tgfb3 | 0 | 0 | 7.27300183 | 0 | 0 | 7.34148597 |
| Tnfrsf1a | 8.90379373 | 7.13050062 | 8.48751907 | 8.869291 | 10.08512 | 9.56614844 |
| Tnfrsf1b | 8.00152361 | 6.49040287 | 9.95513535 | 9.15449888 | 2.53578357 | 6.5261916 |
| Tnfrsf21 | 4.84351147 | 0 | 4.60229475 | 4.67842921 | 5.52125012 | 6.58500292 |
| Tnfsf10 | 5.57895478 | 0 | 0 | 6.17029357 | 8.11110849 | 3.52628697 |
| Tnfsf12 | 0 | 0 | 0 | 5.66296916 | 5.15470027 | 2.81029519 |
| Tob1 | 6.60883404 | 4.71028925 | 6.61940548 | 0 | 7.53391259 | 0 |
| vWF | 6.42109411 | 0 | 7.67992352 | 6.67113351 | 6.93148562 | 7.2346756 |
| Zbtb20 | 9.18932471 | 11.395783 | 9.15649836 | 8.61284336 | 8.06915897 | 9.60060809 |
| Zbtb38 | 7.24785674 | 4.49081527 | 7.78800121 | 7.85959557 | 7.66905166 | 8.13608089 |
| Zfp532 | 0 | 0 | 0 | 0 | 0 | 0 |
| Zfp612 | 9.06730892 | 6.8781252 | 7.30966311 | 9.19853084 | 2.55278286 | 8.83891365 |
| Zfpm1 | 0 | 0 | 7.6939382 | 5.55204554 | 0 | 8.02880897 |
| Zhx2 | 0 | 6.41697281 | 7.21040835 | 0 | 5.66262749 | 9.35665478 |

TABLE 6-2
Single cell expression data (reduced list)-Control

| | | | | HSC- | | |
|---|---|---|---|---|---|---|
| Factor | HSC-Host7 | HSC-Host8 | HSC-Host9 | Host10 | CLP1 | CLP2 |
| Actb | 14.6718473 | 13.3708842 | 14.0765648 | 14.5363732 | 15.5720296 | 15.6020418 |
| Aebp2 | 6.934218 | 5.38858023 | 6.92870369 | 6.83990914 | 6.91310458 | 6.13397519 |
| Ahr | 6.67106288 | 0 | 0 | 0 | 0 | 0 |
| Akt1 | 8.78938258 | 10.6910195 | 9.8127768 | 10.8956807 | 10.5882487 | 9.71594698 |
| Akt2 | 6.75253581 | 3.62756205 | 0 | 6.81240671 | 0 | 5.50111064 |
| Akt3 | 8.32305076 | 5.46246892 | 6.80790868 | 6.46650561 | 8.93439362 | 7.9618537 |
| APC | 0 | 6.36004551 | 0 | 6.14208966 | 3.44926722 | 0 |
| Bad | 0 | 0 | 0 | 0 | 0 | 0 |
| Bax | 0 | 8.20505106 | 7.76032108 | 10.25022 | 10.2921476 | 8.60030468 |
| Bcl11a | 7.92077667 | 3.60167833 | 0 | 0 | 0 | 0 |
| Bcl11b | 0 | 0 | 0 | 0 | 0 | 0 |
| Bcl2 | 4.96817114 | 5.18391882 | 5.86834513 | 4.77451604 | 0 | 0 |
| Bcl211 | 10.2036955 | 9.4735452 | 9.29507619 | 9.23047931 | 10.060975 | 7.87502531 |
| Bcl2111 | 0 | 0 | 0 | 8.25557161 | 0 | 0 |
| Bmi1 | 9.60604305 | 6.56999362 | 7.5702476 | 8.14038399 | 7.42571732 | 7.00110773 |
| Brd3 | 2.43074124 | 7.93247983 | 5.487038 | 7.62759044 | 11.1411249 | 9.66763681 |
| Casp8 | 8.13383235 | 8.73409 | 8.17193114 | 9.06003622 | 9.92872956 | 9.74113972 |
| Casp9 | 8.4257186 | 7.57293558 | 7.8464349 | 7.80792483 | 8.37487536 | 0 |
| Cbx2 | 7.07511053 | 4.48424451 | 5.84700109 | 6.23176944 | 0 | 6.13244563 |
| Cbx8 | 0 | 0 | 0 | 4.43331023 | 2.09486638 | 0 |
| Ccnc | 0 | 6.2797398 | 0 | 6.38691873 | 6.07677146 | 7.90773679 |
| Ccnd1 | 10.0212014 | 0 | 9.34071635 | 0 | 8.62709974 | 0 |
| Ccne2 | 0 | 6.53512964 | 6.54945811 | 6.0438482 | 7.34684561 | 6.25723346 |
| CD34 | 0.01674269 | 7.67391972 | 0 | 10.7870089 | 0 | 0 |
| CD41 | 0 | 0 | 0 | 8.09312343 | 0 | 0 |
| CD48 | 0 | 0 | 0 | 8.10107986 | 10.5431066 | 4.18270305 |
| CD52 | 0 | 3.64518416 | 0 | 0 | 5.65535037 | 8.4769989 |
| CD53 | 8.91469588 | 0 | 10.1863121 | 10.1806135 | 11.1188968 | 10.5349358 |
| CD55 | 7.2980864 | 7.31878302 | 0 | 6.29391433 | 1.43412606 | 6.99636364 |
| CD63 | 8.51246386 | 6.54126666 | 7.37134704 | 6.37418902 | 0 | 0 |
| CD9 | 8.74271831 | 0 | 8.72127967 | 8.8170788 | 0 | 0 |
| Cdc42 | 11.9094394 | 11.5894082 | 11.1126665 | 12.1006451 | 13.0861829 | 12.2864927 |
| Cdk1 | 2.68752057 | 0 | 0 | 11.8397661 | 11.3123555 | 0 |
| Cdk4 | 8.12335302 | 7.87079584 | 7.5720236 | 9.24576955 | 10.3762179 | 10.4600518 |
| Cdkn2b | 0 | 0 | 0 | 0.35740427 | 0 | 0 |
| Cebpa | 0 | 0 | 5.63552878 | 0 | 0 | 0 |
| Csf1r | 0 | 0 | 0 | 0 | 6.27133994 | 5.26584779 |
| Ctnnb1 | 6.79339335 | 7.40629301 | 6.87918414 | 8.36101904 | 5.95935578 | 8.05082722 |
| Cycs | 10.0442638 | 7.54030732 | 9.0344585 | 10.6654921 | 11.2529958 | 11.2582352 |
| Dach1 | 0 | 9.84505342 | 7.97799952 | 11.9672696 | 0 | 0 |
| Dnmt1 | 8.50686835 | 7.570001 | 3.23481103 | 10.5464652 | 12.6178625 | 12.0559888 |
| Dnmt3a | 10.0573123 | 9.34977288 | 8.47634202 | 10.5147996 | 8.06454655 | 9.25761414 |
| Dnmt3b | 8.08236706 | 7.77693525 | 7.43902731 | 6.35981456 | 8.61270517 | 0 |
| Dtx1 | 0 | 1.20990211 | 0 | 2.35858319 | 0 | 0 |
| Dtx4 | 0 | 0.84530668 | 0 | 8.42626641 | 0 | 0 |
| Ebf1 | 0 | 0 | 0 | 0 | 10.5975489 | 11.2372886 |
| Ep300 | 8.67464583 | 9.2042527 | 8.90097872 | 9.29742804 | 8.73799831 | 8.9933198 |
| Epor | 7.4651798 | 7.99907556 | 7.67252065 | 7.98170347 | 0.10277376 | 4.78402129 |
| Erg | 11.1082009 | 7.23780514 | 10.3502921 | 10.2615194 | 12.9408351 | 11.0993994 |
| Esr1 | 8.54768834 | 7.99110915 | 6.24818597 | 9.62048384 | 10.4231044 | 0 |
| ETS1 | 6.86365699 | 4.84774761 | 8.3168225 | 6.6480974 | 13.8494997 | 11.6438204 |
| ETS2 | 7.64755071 | 7.54891501 | 0 | 8.17449216 | 0 | 0 |
| Etv3 | 5.78507161 | 0 | 5.75634937 | 3.75032653 | 4.76128972 | 2.70875229 |
| Etv6 | 8.82488989 | 10.4027054 | 10.0840126 | 12.226941 | 10.5939014 | 9.97978593 |
| Ezh2 | 6.34735252 | 4.06993896 | 5.66118811 | 8.83156708 | 11.5011279 | 10.4172165 |
| Fas | 0 | 0 | 5.0587006 | 0 | 0 | 0 |
| Fcgr2b | 5.48237699 | 1.56950279 | 6.50908621 | 6.14234211 | 3.36211875 | 0 |
| Fcgr3 | 0 | 0 | 0 | 0 | 0 | 0 |
| Fli1 | 10.6505478 | 9.64542823 | 11.1441998 | 11.6211551 | 10.9483997 | 10.3713463 |
| Flt3 | 0 | 0 | 0 | 9.55475223 | 0 | 0 |
| Fosl1 | 0 | 0 | 0 | 1.86707308 | 0 | 8.47337507 |
| Foxo1 | 7.87606422 | 9.05152117 | 9.80912191 | 11.1420747 | 11.6728318 | 10.918137 |
| Foxo3 | 8.4243012 | 7.7040044 | 9.07363846 | 9.75726551 | 6.51553987 | 6.92529651 |
| Gapdh | 7.84932494 | 8.15466782 | 8.21027854 | 8.00493653 | 12.3780006 | 11.3641618 |
| Gata1 | 0 | 0 | 1.32627298 | 4.99268331 | 0 | 0 |
| Gata2 | 7.1358369 | 7.84253879 | 7.5357683 | 4.15447711 | 0 | 0 |
| Gata3 | 9.23864702 | 7.08926856 | 7.70423652 | 9.1691048 | 6.33257429 | 0 |
| Gfi1 | 8.4722437 | 0 | 2.45881453 | 8.01637799 | 0 | 6.71345188 |
| Gfi1b | 9.78145684 | 0 | 0 | 7.14731375 | 0 | 0 |
| Hes5 | 0 | 0 | 0 | 0 | 0 | 0 |
| Hey1 | 0 | 0 | 0 | 0.50104001 | 0 | 0 |
| Hlf | 10.4196373 | 7.93837692 | 9.25512238 | 9.64501202 | 0 | 0 |
| Id2 | 0 | 0 | 0 | 0.37307203 | 0 | 0 |
| Ifi203 | 11.2385326 | 10.675148 | 11.1293957 | 11.5993821 | 13.2875382 | 10.2274453 |
| Ifi205 | 0 | 0 | 0 | 0 | 0 | 0 |
| Ifitm1 | 11.8294232 | 11.1006374 | 12.8299047 | 11.7081516 | 0 | 0 |
| Ikzf1 | 10.4603278 | 7.9081258 | 8.39039117 | 9.30500104 | 11.2708394 | 10.4757841 |
| Ikzf2 | 8.66069698 | 0 | 8.07815335 | 9.24251035 | 0 | 0 |
| Il7R | 0 | 0 | 0 | 0 | 3.86371591 | 4.80700829 |
| Irf4 | 0 | 0 | 0 | 0 | 9.2290601 | 10.2309003 |
| Irf6 | 2.64609076 | 0 | 4.55767937 | 4.22209488 | 0 | 0 |
| Irf8 | 0 | 1.57386134 | 0 | 8.84149401 | 0 | 8.81600274 |
| Kdr | 0 | 0 | 0 | 0 | 0 | 0 |
| Kit | 12.2681758 | 11.1853776 | 11.5755541 | 11.3487544 | 10.3091102 | 9.16742564 |
| Klf1 | 0 | 0 | 5.19001782 | 2.69496283 | 0 | 0 |
| Klf12 | 8.99195223 | 6.89401764 | 0 | 0 | 4.77266959 | 7.98400431 |
| Ldb1 | 10.7730297 | 9.4520141 | 9.55889768 | 9.47012092 | 8.99931122 | 10.47084 |
| Lin28a | 6.21043595 | 5.10100157 | 8.34850576 | 7.64045938 | 7.50871774 | 9.03894646 |
| Lmo2 | 11.5565524 | 9.01389959 | 10.9404097 | 10.1650659 | 4.46826015 | 6.2900714 |
| Ly6a | 10.274331 | 8.62489906 | 10.9730888 | 0.67547765 | 0 | 0 |
| Lyl1 | 3.44144381 | 7.53639677 | 6.92249445 | 8.41401114 | 7.99916677 | 8.4577076 |
| Mbd2 | 7.07180263 | 8.80305911 | 9.83435118 | 7.32171913 | 11.0889587 | 11.1378285 |
| Meis1 | 7.80771805 | 6.57260088 | 8.3801574 | 6.64771096 | 0 | 5.32655256 |
| Mllt3 | 5.27987488 | 4.98216842 | 0 | 4.98006428 | 0 | 0.43104733 |
| Mpl | 9.95026098 | 9.29878047 | 10.5382189 | 8.92503515 | 0 | 0 |
| Muc13 | 9.58693895 | 5.98850625 | 10.5817646 | 10.34105 | 0 | 0 |
| Myb | 11.9113929 | 11.3263068 | 9.38747922 | 12.0083232 | 13.2716596 | 13.3551636 |
| Myc | 0 | 7.55865639 | 5.71326556 | 9.60742235 | 0 | 7.03978632 |
| Mycn | 9.2475789 | 11.2225067 | 12.0059366 | 9.17037192 | 0 | 0 |
| Ndn | 9.34022589 | 8.94700354 | 8.72830108 | 7.25627641 | 0 | 0 |
| Nfat5 | 10.9266838 | 10.3886042 | 10.2456748 | 9.51279929 | 3.18257792 | 0 |
| Nfia | 9.8356555 | 8.60236457 | 8.92289712 | 10.0014286 | 8.2885559 | 0 |
| Nfkb1 | 0 | 0 | 4.48890776 | 0 | 3.74973604 | 0 |
| Notch1 | 7.66102275 | 0 | 6.91201627 | 8.32291131 | 7.91814495 | 7.36965349 |
| Pax4 | 0 | 0 | 0 | 0 | 0 | 0 |
| Pax5 | 0 | 0 | 0 | 0 | 9.67689902 | 11.6203933 |
| Pax9 | 0 | 0 | 0 | 0.57036927 | 4.48973549 | 0 |
| Pbx1 | 5.69269047 | 0 | 5.43069763 | 0 | 0 | 0 |
| PIk3ca | 0 | 7.18092062 | 7.27208139 | 9.05710063 | 9.40185149 | 9.55052543 |
| PIk3R2 | 0 | 0 | 7.5160141 | 8.56807024 | 9.73539407 | 0 |
| Plag1 | 7.73898932 | 7.96365738 | 8.07352148 | 0 | 0 | 0 |
| Prf1 | 0 | 0 | 0 | 0 | 0 | 0 |
| Pten | 10.1342741 | 9.78469549 | 9.33811703 | 11.1785408 | 10.1894192 | 10.4359312 |
| Rb1 | 9.29604621 | 9.27765839 | 7.51678183 | 8.27880038 | 11.9054276 | 10.9424567 |
| Rora | 6.10890584 | 7.3877893 | 8.15836998 | 5.4939429 | 0 | 0 |
| Runx1 | 0 | 7.76888704 | 8.78603048 | 7.67062362 | 8.305547 | 0 |
| Runx2 | 0 | 3.79386494 | 3.6008219 | 5.35557258 | 0 | 0 |
| Satb1 | 0 | 0 | 0 | 8.99400379 | 10.1837922 | 8.39346313 |
| Sdpr | 5.78136407 | 4.21076733 | 0 | 0.82691288 | 0 | 0 |
| Sell | 0 | 0 | 1.61946707 | 0 | 0 | 0 |
| Sfpi1 | 10.0042663 | 9.37371199 | 9.15518065 | 9.65832452 | 0 | 9.26882608 |
| Slamf1 | 7.81411202 | 6.8594725 | 7.95128279 | 0 | 0 | 0 |
| Smarca4 | 10.3380905 | 7.42905599 | 9.2510329 | 11.5218685 | 14.4938783 | 13.4081997 |
| Sos1 | 0 | 0 | 6.5261554 | 6.79179662 | 0 | 5.43289492 |
| Stat1 | 1.71494059 | 0 | 0 | 3.42562416 | 5.64062199 | 0 |
| Stat3 | 10.7412032 | 8.92068828 | 8.96113036 | 10.4989945 | 8.68504508 | 8.21020662 |
| Stat4 | 9.21395012 | 9.36252836 | 9.57705104 | 8.5317536 | 0 | 0.65364229 |
| Stat6 | 8.27498229 | 8.51520973 | 8.34381559 | 8.60680209 | 10.1139186 | 9.61023286 |
| Suz12 | 8.36186765 | 7.85222591 | 8.01568165 | 9.19083991 | 12.1912291 | 10.7847116 |
| Tal1 | 1.22646608 | 0 | 0 | 0.85919234 | 8.29002547 | 0 |
| Tcf3 | 0 | 10.0641005 | 0 | 0 | 10.2329064 | 9.57044442 |
| Tcf4 | 10.3945958 | 8.86390901 | 9.93214915 | 10.6432336 | 11.5584564 | 11.0576929 |
| Tcf7 | 1.59196764 | 0 | 0.92915579 | 0 | 0 | 5.45500333 |
| Tek | 0 | 0 | 0 | 7.77878275 | 0 | 0 |
| Tfrc | 4.90970417 | 8.02894875 | 7.93433882 | 7.81882114 | 10.1158882 | 10.2735536 |
| Tgfb1 | 0 | 3.32919416 | 5.90260252 | 3.25808206 | 3.7705399 | 0 |
| Tgfb2 | 0 | 0 | 0 | 3.22432655 | 0 | 3.37538454 |
| Tgfb3 | 0 | 6.69135338 | 1.40782238 | 3.95650619 | 0 | 0 |
| Tnfrsf1a | 9.92981833 | 7.3738534 | 8.64338251 | 8.24251812 | 0 | 0 |
| Tnfrsf1b | 8.93673702 | 9.48765082 | 9.5506678 | 6.21083423 | 3.78885776 | 3.73572941 |
| Tnfrsf2l | 4.89969433 | 0 | 6.93921933 | 7.10963898 | 0 | 0 |
| Tnfsf10 | 7.10728827 | 0 | 0 | 1.58582089 | 7.14613579 | 8.05630727 |
| Tnfsf12 | 3.38261217 | 0 | 2.19082075 | 0 | 0 | 0 |
| Tob1 | 0 | 5.20593174 | 0 | 0 | 0 | 0 |
| vWF | 4.95948597 | 6.28053967 | 5.43694051 | 0 | 0 | 0 |
| Zbtb20 | 9.61893778 | 9.81916761 | 9.00655347 | 7.72955135 | 0 | 0 |
| Zbtb38 | 9.10026874 | 6.185996 | 7.56423848 | 6.82663886 | 7.73312626 | 3.84361329 |
| Zfp532 | 0 | 0 | 0 | 0 | 0.10416971 | 0 |
| Zfp612 | 5.28324577 | 6.48139199 | 8.74136356 | 5.56744079 | 0 | 6.50143494 |
| Zfpm1 | 8.58664951 | 6.0911617 | 8.1830324 | 0 | 6.44606012 | 5.62364305 |
| Zhx2 | 7.56629134 | 7.63051187 | 0 | 5.24483627 | 0 | 0 |

TABLE 6-3
Single cell expression data (reduced list)-Control

| Factor | CLP3 | CLP4 | CLP5 | CLP6 | CLP7 | CLP8 |
|---|---|---|---|---|---|---|
| Actb | 13.4721085 | 15.2351724 | 15.2719547 | 16.31177 | 16.919695 | 17.0516789 |
| Aebp2 | 4.45141147 | 4.38441532 | 7.10616819 | 6.49378333 | 7.1531144 | 5.6116867 |
| Ahr | 0 | 0 | 7.00481198 | 0 | 0 | 0 |
| Akt1 | 7.3884758 | 9.17609503 | 9.55146467 | 10.0057847 | 10.2031478 | 11.1623017 |
| Akt2 | 1.87065597 | 0 | 0 | 0 | 7.27787365 | 0 |
| Akt3 | 7.14641592 | 0 | 0 | 8.91809255 | 8.53101085 | 8.95553865 |
| APC | 0 | 0 | 7.27741159 | 0 | 9.72461612 | 0 |
| Bad | 0 | 0 | 0 | 0 | 0 | 0 |
| Bax | 5.64368167 | 7.7793443 | 7.96170511 | 9.7217077 | 11.9875259 | 11.9783765 |
| Bcl11a | 0 | 0 | 0 | 0 | 8.6331668 | 9.1297033 |
| Bcl11b | 0 | 0 | 0 | 8.64946621 | 0 | 0 |
| Bcl2 | 0 | 0 | 0 | 4.47644651 | 4.63608396 | 0 |
| Bcl211 | 4.6189348 | 0 | 10.2286999 | 10.9686351 | 10.604158 | 11.3030776 |
| Bcl2111 | 4.8989012 | 0 | 8.32168555 | 0 | 0 | 0 |
| Bmi1 | 3.17094341 | 6.36759845 | 5.13831255 | 6.9969786 | 8.36369633 | 7.04410175 |
| Brd3 | 6.59116273 | 8.85891039 | 10.3417165 | 10.3202288 | 11.5288449 | 11.1568732 |
| Casp8 | 9.02211423 | 8.05947856 | 9.77788318 | 10.1196359 | 11.9218075 | 10.3568659 |
| Casp9 | 5.06149028 | 0 | 0 | 0 | 8.30557433 | 9.75192608 |
| Cbx2 | 4.42759599 | 7.57182896 | 2.65329776 | 8.35205791 | 6.1484868 | 7.77479327 |
| Cbx8 | 0 | 0 | 0 | 7.10684953 | 0 | 0 |
| Ccnc | 3.70061852 | 7.15959988 | 8.92627786 | 8.61131431 | 9.6072497 | 9.48325249 |
| Ccnd1 | 0 | 2.93758213 | 0 | 0 | 10.6400803 | 0 |
| Ccne2 | 5.21666008 | 7.17885114 | 11.5186474 | 0 | 9.77794018 | 10.5222899 |
| CD34 | 0 | 0 | 0 | 0 | 0 | 0 |
| CD41 | 6.34043371 | 0 | 0 | 0 | 0 | 0 |
| CD48 | 0 | 7.57200005 | 9.20489806 | 9.11301325 | 12.225357 | 9.60365514 |
| CD52 | 7.65018871 | 7.48017023 | 7.43352856 | 0 | 12.104936 | 12.1008653 |
| CD53 | 10.1411695 | 7.84826499 | 9.96783218 | 10.4527685 | 10.929522 | 11.4800078 |
| CD55 | 7.0314255 | 0 | 0 | 0 | 0 | 0 |
| CD63 | 0 | 0 | 0 | 0 | 0 | 0 |
| CD9 | 0 | 0 | 0 | 0 | 0 | 7.60428115 |
| Cdc42 | 11.4392736 | 12.714625 | 12.1761207 | 13.5034801 | 13.8493379 | 13.7053792 |
| Cdk1 | 6.69762232 | 0 | 9.85122167 | 11.4158803 | 12.1196679 | 12.4467872 |
| Cdk4 | 5.98607517 | 6.97494046 | 9.31645941 | 10.220209 | 12.7159863 | 12.2210916 |
| Cdkn2b | 0 | 0 | 0 | 0 | 0 | 0 |
| Cebpa | 0 | 3.58268727 | 0 | 0 | 0 | 0 |
| Csf1r | 0 | 0 | 1.65538427 | 3.97435095 | 8.52442108 | 0 |
| Ctnnb1 | 3.62240099 | 6.62276734 | 7.86637465 | 7.51682333 | 9.83553487 | 10.0053905 |
| Cycs | 9.02261009 | 11.2219931 | 12.0781554 | 10.7960042 | 14.1072249 | 14.0649415 |
| Dach1 | 0 | 0 | 0 | 0 | 0 | 0 |
| Dnmt1 | 9.22693253 | 9.45595878 | 12.0119534 | 12.2094736 | 13.5638023 | 13.6951805 |
| Dnmt3a | 10.1899327 | 10.0717063 | 9.85756039 | 6.45117101 | 8.59850296 | 10.3104357 |
| Dnmt3b | 0 | 0 | 0 | 8.47080648 | 9.14187427 | 5.69957905 |
| Dtx1 | 1.994687 | 4.40399225 | 3.53694035 | 1.04383263 | 0.47312172 | 3.02752053 |
| Dtx4 | 3.40004889 | 0 | 2.47750396 | 0 | 9.02141488 | 8.78305504 |
| Ebf1 | 9.85337813 | 10.0549087 | 10.0192028 | 10.3755802 | 10.3006671 | 10.0964241 |
| Ep300 | 8.7177225 | 7.49266991 | 9.85202509 | 10.4082795 | 9.68961902 | 9.97406922 |
| Epor | 2.12061309 | 0 | 3.84685187 | 4.15570632 | 3.80975151 | 5.26571959 |
| Erg | 9.92070322 | 10.2435688 | 11.5232616 | 11.7222598 | 12.279183 | 12.5339555 |
| Esr1 | 8.69677383 | 10.4600212 | 10.205356 | 8.31154408 | 7.71734777 | 0 |
| ETS1 | 11.3057093 | 12.1559856 | 12.6586051 | 12.5933092 | 12.1381441 | 12.9889476 |
| ETS2 | 0 | 0 | 9.6997688 | 8.36290987 | 8.2095168 | 0.73462164 |
| Etv3 | 0 | 3.38933838 | 0 | 3.43657627 | 6.61600906 | 2.44247804 |
| Etv6 | 0 | 0 | 8.65286731 | 10.7013694 | 10.9628988 | 10.3361814 |
| Ezh2 | 8.02471927 | 8.50978683 | 12.1912021 | 10.8533753 | 11.493762 | 11.5119798 |
| Fas | 0 | 0 | 0 | 0 | 0 | 0 |
| Fcgr2b | 7.6797349 | 0 | 0 | 0 | 0 | 0 |
| Fcgr3 | 0 | 0 | 0 | 6.10259634 | 0 | 0 |
| Fli1 | 9.93711884 | 10.9464019 | 11.1285519 | 9.54487089 | 10.8365241 | 11.5533691 |
| Flt3 | 0 | 0 | 0 | 0 | 9.56640355 | 10.3432711 |
| Fosl1 | 0 | 0 | 0 | 0 | 0 | 0 |
| Foxo1 | 11.0966868 | 8.79275995 | 11.8050162 | 12.7164993 | 12.8446053 | 12.3408678 |
| Foxo3 | 3.57817888 | 6.51216426 | 0 | 6.58016006 | 7.771922 | 8.46989317 |
| Gapdh | 5.85168672 | 10.6505893 | 12.1850341 | 12.4040061 | 13.3572594 | 13.192243 |
| Gata1 | 6.47274743 | 0 | 0 | 2.62704169 | 3.52126724 | 0 |
| Gata2 | 0 | 0 | 0.36206896 | 0 | 0 | 0 |
| Gata3 | 0 | 0 | 0 | 0 | 0 | 0 |
| Gfi1 | 5.89645562 | 0 | 8.17908872 | 0 | 4.89958389 | 2.83324318 |
| Gfi1b | 7.35569282 | 0 | 0 | 0 | 0 | 0 |
| Hes5 | 2.85354691 | 0 | 0 | 0 | 6.33604471 | 0 |
| Hey1 | 0 | 0 | 0 | 0 | 0 | 0 |
| Hlf | 0 | 0 | 0 | 0 | 0 | 0 |
| Id2 | 0 | 0 | 0 | 0 | 0 | 0 |
| Ifi203 | 11.7954894 | 10.973362 | 11.001131 | 11.4270334 | 12.5609017 | 10.7759677 |
| Ifi205 | 0 | 0 | 0 | 0 | 0 | 8.09318704 |
| Ifitm1 | 0 | 0 | 0 | 2.84027402 | 0 | 0 |
| Ikzf1 | 9.73388122 | 8.31161283 | 9.89390965 | 8.89596541 | 11.5318373 | 11.7353046 |
| Ikzf2 | 7.68319581 | 0 | 0 | 0 | 0 | 0 |
| Il7R | 3.50218592 | 3.36711209 | 5.2921046 | 4.8044562 | 5.550561 | 6.76651483 |
| Irf4 | 9.79482653 | 0 | 9.58168074 | 8.08809386 | 7.5643288 | 2.25516181 |
| Irf6 | 0 | 4.76893306 | 4.55078055 | 0 | 2.82795862 | 2.04839193 |
| Irf8 | 7.95992816 | 10.1806094 | 7.75876351 | 8.80670344 | 11.7480118 | 9.73229364 |
| Kdr | 0 | 0 | 0 | 0 | 0 | 0 |
| Kit | 0.53419079 | 0 | 9.96379129 | 10.7375717 | 10.2201977 | 9.16826777 |
| Klf1 | 6.82013214 | 0 | 0 | 1.0024718 | 0 | 0 |
| Klf12 | 0 | 0 | 0 | 0 | 0 | 0 |
| Ldb1 | 9.90431329 | 9.96028836 | 11.2260518 | 9.83927772 | 11.895041 | 11.7935625 |
| Lin28a | 5.35085436 | 7.33632529 | 6.44890786 | 6.34118404 | 6.36516284 | 9.37400697 |
| Lmo2 | 6.46868712 | 4.61214257 | 5.14599266 | 5.60258194 | 6.56246105 | 3.9775212 |
| Ly6a | 0 | 3.53194881 | 0 | 0.37013501 | 7.4460115 | 10.5913393 |
| Lyl1 | 9.3983485 | 8.54480739 | 7.34706955 | 9.10668449 | 11.3876375 | 7.64786048 |
| Mbd2 | 8.94182953 | 9.36449253 | 10.2060984 | 9.52243477 | 11.5407023 | 12.2595821 |
| Meis1 | 0 | 0 | 5.21224582 | 5.79085752 | 5.40464488 | 0 |
| Mllt3 | 0 | 0 | 0 | 2.11014429 | 0 | 2.59630677 |
| Mpl | 0 | 0 | 0 | 4.02311498 | 0 | 0 |
| Muc13 | 0 | 2.57260911 | 0 | 0 | 0 | 0 |
| Myb | 12.3033699 | 12.488897 | 12.3730793 | 12.3171025 | 13.0048416 | 12.7052775 |
| Myc | 5.93099913 | 11.6265583 | 0 | 0 | 14.0060868 | 10.9410236 |
| Mycn | 0 | 0 | 0 | 0 | 0 | 0 |
| Ndn | 0 | 0 | 0 | 0 | 0 | 0 |
| Nfat5 | 7.24590475 | 5.59931195 | 10.8263667 | 6.57678171 | 8.07891887 | 6.14435558 |
| Nfia | 8.37013642 | 8.26157976 | 10.2847505 | 8.23082089 | 0 | 8.96451019 |
| Nfkb1 | 0 | 0 | 4.99179474 | 0 | 3.6973326 | 6.1512888 |
| Notch1 | 8.10251427 | 0 | 0 | 0 | 8.45173916 | 8.82084626 |
| Pax4 | 0 | 0 | 0 | 4.36397603 | 3.43221858 | 0 |
| Pax5 | 9.34367693 | 9.92404452 | 9.77304 | 11.0122144 | 10.3872408 | 10.8331107 |
| Pax9 | 0 | 0 | 5.18709971 | 0 | 3.29966428 | 0 |
| Pbx1 | 0 | 0 | 0 | 0 | 0 | 0 |
| PIk3ca | 9.63937118 | 6.79728215 | 11.3857624 | 10.7462144 | 9.15262138 | 10.9538129 |
| PIk3R2 | 7.90901728 | 7.26209506 | 8.54304817 | 8.37704722 | 9.50572232 | 9.62140977 |
| Plag1 | 0 | 0 | 0 | 5.97796547 | 0 | 0 |
| Prf1 | 0 | 0 | 0 | 0 | 0 | 0 |
| Pten | 9.72285323 | 11.0091543 | 10.636038 | 10.0259098 | 11.7798461 | 10.8939695 |
| Rb1 | 9.00979222 | 8.85052189 | 11.0074341 | 12.0368206 | 11.2827 | 12.2052216 |
| Rora | 0 | 0 | 0 | 0 | 0 | 0 |
| Runx1 | 3.35520365 | 8.41018156 | 0 | 7.20098788 | 10.3169336 | 7.21605593 |
| Runx2 | 0 | 0 | 0 | 0 | 0 | 0 |
| Satb1 | 10.3474498 | 0 | 10.4087951 | 10.4125548 | 11.5917762 | 10.8352979 |
| Sdpr | 0 | 0 | 0 | 0 | 0 | 0 |
| Sell | 0 | 0 | 0 | 9.4220848 | 11.0820261 | 0 |
| Sfpi1 | 0 | 6.01015121 | 9.2965798 | 0 | 9.91399926 | 8.59032855 |
| Slamf1 | 0 | 0 | 0 | 5.98712463 | 0 | 0 |
| Smarca4 | 13.439393 | 12.5294897 | 14.6724616 | 15.0680818 | 14.5786721 | 13.7911882 |
| Sos1 | 0.97380716 | 6.19138786 | 0 | 5.38334215 | 7.45674234 | 7.50591767 |
| Stat1 | 0.7689796 | 6.50704145 | 0 | 0.30611506 | 2.57411315 | 0 |
| Stat3 | 0 | 8.93991247 | 0 | 6.7379161 | 10.502702 | 0 |
| Stat4 | 6.56531371 | 6.26156325 | 7.27133959 | 8.37209933 | 7.78457398 | 6.6457098 |
| Stat6 | 7.7239777 | 8.43459593 | 9.892434 | 9.03877839 | 10.1786368 | 3.86022053 |
| Suz12 | 9.22489651 | 0 | 10.0290041 | 12.3349832 | 12.611291 | 13.0733851 |
| Tal1 | 0 | 0 | 6.67626014 | 6.82238434 | 7.45135976 | 3.68581347 |
| Tcf3 | 3.3752031 | 0 | 7.69136582 | 8.65824457 | 9.6940747 | 8.57311453 |
| Tcf4 | 11.1561631 | 9.47548756 | 10.1792855 | 11.8284673 | 11.6158594 | 10.8851719 |
| Tcf7 | 0 | 0 | 1.68581989 | 0 | 1.46116868 | 6.50226768 |
| Tek | 0 | 0 | 0 | 0 | 0 | 0 |
| Tfrc | 8.384231 | 8.62609735 | 8.72228476 | 9.79712611 | 12.2298851 | 12.6617066 |
| Tgfb1 | 0 | 1.30714129 | 0 | 0 | 8.57409133 | 4.42951853 |
| Tgfb2 | 0 | 0 | 0 | 0 | 0 | 0 |
| Tgfb3 | 0 | 0 | 0 | 5.906968 | 6.8247631 | 0 |
| Tnfrsf1a | 0 | 0 | 8.13776036 | 0 | 6.6654212 | 0 |
| Tnfrsf1b | 5.48788691 | 0 | 0 | 0 | 8.83222639 | 0 |
| Tnfrsf21 | 0 | 3.83171313 | 4.44763219 | 5.66301599 | 6.31162299 | 5.70640904 |
| Tnfsf10 | 0 | 0 | 7.49803338 | 0 | 7.18042827 | 0 |
| Tnfsf12 | 0 | 0 | 0 | 0 | 0 | 0 |
| Tob1 | 0 | 4.40571001 | 0 | 0 | 0 | 0 |
| vWF | 0 | 0 | 0 | 0 | 0 | 0 |
| Zbtb20 | 8.29135619 | 0 | 8.47708838 | 0 | 0 | 8.27839243 |
| Zbtb38 | 8.58554038 | 0.99042294 | 8.02102069 | 0 | 0 | 7.24565903 |
| Zfp532 | 0 | 3.87621119 | 2.9154077 | 0 | 4.19402652 | 3.24319594 |
| Zfp612 | 1.03716649 | 0 | 2.11894576 | 6.50227904 | 7.64231508 | 7.61374585 |
| Zfpm1 | 6.66189343 | 0 | 3.0682001 | 0 | 0 | 0 |
| Zhx2 | 8.44133547 | 0 | 0 | 0 | 0 | 9.15911003 |

TABLE 6-4
Single cell expression data (reduced list)-Control

| Factor | CLP9 | CLP10 | CMP1 | CMP2 | CMP3 | CMP4 |
|---|---|---|---|---|---|---|
| Actb | 16.7472085 | 16.8352612 | 16.8602626 | 16.1110931 | 14.4827986 | 15.0603357 |
| Aebp2 | 5.10557045 | 3.3120632 | 5.90217636 | 5.99828664 | 4.16296449 | 5.95408203 |
| Ahr | 7.89043699 | 0 | 0 | 0 | 0 | 0 |
| Akt1 | 8.18148335 | 8.76665238 | 9.82206378 | 10.7068971 | 8.0750109 | 9.71182542 |
| Akt2 | 0 | 0 | 4.73623383 | 5.90460679 | 0 | 5.31671466 |
| Akt3 | 7.62109377 | 8.60100117 | 10.3161486 | 9.89323892 | 7.25420238 | 7.89506854 |
| APC | 0 | 0 | 0 | 6.42364613 | 0 | 1.66166347 |
| Bad | 0 | 0 | 0 | 0 | 0 | 0 |
| Bax | 9.29238441 | 8.3822507 | 9.02204677 | 9.89324281 | 0 | 8.05690985 |
| Bcl11a | 10.3227685 | 0 | 0 | 0 | 0 | 0 |
| Bcl11b | 4.17625304 | 3.92709271 | 0 | 0 | 0 | 6.87178744 |
| Bcl2 | 5.16525658 | 0 | 0 | 0 | 0 | 7.99225602 |
| Bcl211 | 8.3489033 | 9.55544552 | 0 | 8.39669119 | 0 | 0 |
| Bcl2111 | 0 | 4.95609125 | 9.99775747 | 9.90050891 | 8.99255245 | 2.85336974 |
| Bmi1 | 7.02747752 | 7.05328898 | 6.44377861 | 6.35815343 | 0 | 5.61256235 |
| Brd3 | 10.4902324 | 10.3566216 | 9.01263098 | 11.3736884 | 9.51822117 | 10.0173723 |
| Casp8 | 10.3220679 | 10.7369556 | 9.56591918 | 12.353426 | 10.3690709 | 10.4324467 |
| Casp9 | 0 | 0 | 0 | 8.91438552 | 0 | 9.50719509 |
| Cbx2 | 5.63357469 | 5.32126348 | 0 | 6.26420923 | 0 | 4.88635048 |
| Cbx8 | 0 | 4.8985443 | 0 | 0 | 0 | 0 |
| Ccnc | 9.44462333 | 10.6012883 | 8.71922383 | 8.09587133 | 7.39164169 | 7.88535554 |
| Ccnd1 | 13.1309938 | 8.71442109 | 10.4720419 | 7.63908907 | 0 | 7.37626749 |
| Ccne2 | 0 | 8.35161245 | 0 | 7.74541722 | 0 | 0 |
| CD34 | 0 | 0 | 0 | 11.0938464 | 0 | 10.9563356 |
| CD41 | 0 | 0 | 0 | 10.8578571 | 10.6626378 | 0 |
| CD48 | 10.1531953 | 9.61840884 | 11.7599349 | 12.6456392 | 7.70003657 | 10.4526615 |
| CD52 | 11.7226951 | 10.1559179 | 12.0658796 | 10.3906592 | 0 | 7.66859187 |
| CD53 | 12.8012579 | 11.5337875 | 11.257362 | 13.1982289 | 0 | 11.0963127 |
| CD55 | 0 | 0 | 0 | 0 | 8.8819203 | 0 |
| CD63 | 0 | 0 | 6.94398394 | 9.24084619 | 0 | 6.92519888 |
| CD9 | 7.17538049 | 0 | 8.11834259 | 0 | 0 | 7.63859446 |
| Cdc42 | 12.9539909 | 13.4145126 | 14.1395004 | 13.5734692 | 12.5791339 | 12.8894502 |
| Cdk1 | 11.2702793 | 11.3939722 | 0.20875207 | 11.1428913 | 0 | 0 |
| Cdk4 | 8.41570405 | 11.076971 | 6.87263164 | 10.9598136 | 9.6088668 | 10.5827767 |
| Cdkn2b | 0 | 0 | 0 | 0 | 0 | 0 |
| Cebpa | 0.89723358 | 0 | 10.2311173 | 13.4808053 | 8.18762349 | 11.6632459 |
| Csf1r | 0.68220487 | 0 | 8.91048376 | 8.52043829 | 7.87011519 | 9.68797102 |
| Ctnnb1 | 6.632855 | 7.60076967 | 4.83416648 | 8.15260001 | 5.67395641 | 6.9102424 |
| Cycs | 10.3257774 | 11.5926 | 11.9196287 | 13.2793334 | 7.61714986 | 10.435771 |
| Dach1 | 0 | 0 | 0 | 11.8661392 | 9.79500635 | 0 |
| Dnmt1 | 10.9639197 | 10.9779133 | 9.7927147 | 13.2742978 | 6.43285115 | 11.5344213 |
| Dnmt3a | 10.1312258 | 10.6116941 | 0.01680684 | 11.2120611 | 10.1685075 | 9.93533932 |
| Dnmt3b | 6.02587145 | 0 | 0.28023125 | 10.9143614 | 8.14598611 | 11.5847104 |
| Dtx1 | 0 | 0 | 1.92305529 | 0 | 0 | 2.30151059 |
| Dtx4 | 9.68534196 | 5.647952 | 0 | 4.77885166 | 0 | 0 |
| Ebf1 | 0 | 0 | 0 | 0 | 0 | 6.27167952 |
| Ep300 | 10.430224 | 10.5649677 | 10.9844624 | 11.2861422 | 10.0900532 | 10.0078637 |
| Epor | 6.0607173 | 5.65375289 | 6.31948929 | 5.15194981 | 4.40969335 | 2.82619662 |
| Erg | 0 | 0 | 0 | 12.0363518 | 10.0931312 | 10.5218299 |
| Esr1 | 10.9412325 | 8.69857347 | 0 | 8.23822017 | 0 | 8.23908889 |
| ETS1 | 12.3373625 | 12.1142197 | 0 | 0 | 0 | 0 |
| ETS2 | 6.29583632 | 0 | 0 | 0.68650314 | 0 | 6.35073519 |
| Etv3 | 4.3355231 | 4.42802306 | 5.32393809 | 5.87942342 | 0 | 3.92981296 |
| Etv6 | 8.83941501 | 0 | 8.61360798 | 12.0360378 | 10.3250242 | 10.9028847 |
| Ezh2 | 8.85028888 | 10.0605202 | 7.27389146 | 9.32121342 | 7.38296829 | 10.0425905 |
| Fas | 0 | 0 | 0 | 4.30798527 | 7.17965527 | 0 |
| Fcgr2b | 0 | 0 | 7.77302706 | 7.68233416 | 0 | 0 |
| Fcgr3 | 0 | 0 | 2.16280252 | 7.43345552 | 0 | 0 |
| Fli1 | 10.3126762 | 11.0853737 | 8.11430154 | 9.84452071 | 11.0778188 | 10.5409282 |
| Flt3 | 10.8733788 | 11.8851759 | 10.4953795 | 8.72900327 | 0 | 11.9407693 |
| Fosl1 | 0 | 0 | 0 | 0 | 0 | 0 |
| Foxo1 | 12.9862277 | 0 | 9.12833227 | 10.3210046 | 8.57814146 | 10.4483982 |
| Foxo3 | 9.35939781 | 9.17812532 | 7.75264584 | 8.79843273 | 7.38358954 | 9.91435082 |
| Gapdh | 12.0414546 | 10.6649131 | 8.59496634 | 13.2322627 | 9.24678558 | 10.5425893 |
| Gata1 | 2.47237968 | 5.18777488 | 0 | 0 | 9.92586716 | 0 |
| Gata2 | 0 | 1.70095059 | 0 | 5.59893348 | 5.27123302 | 2.16028386 |
| Gata3 | 0 | 5.17595033 | 0 | 0 | 0 | 6.05524501 |
| Gfi1 | 3.35012985 | 0 | 0 | 5.30002147 | 3.26363882 | 6.13120183 |
| Gfi1b | 0 | 8.53467602 | 0 | 10.0611223 | 11.6926351 | 8.75372639 |
| Hes5 | 0 | 0 | 0 | 0 | 0 | 0 |
| Hey1 | 0 | 1.04367745 | 0 | 0 | 0 | 0 |
| Hlf | 0 | 0 | 0 | 7.97611682 | 7.82618822 | 9.46609084 |
| Id2 | 8.21405404 | 0 | 9.70225491 | 5.84854144 | 0 | 4.38699582 |
| Ifi203 | 13.121305 | 11.7715254 | 13.5766403 | 10.4527001 | 10.3475725 | 11.3925667 |
| Ifi205 | 0 | 0 | 12.6181685 | 0 | 0 | 0 |
| Ifitm1 | 9.57706163 | 0 | 0 | 9.63434379 | 11.5761744 | 11.1838971 |
| Ikzf1 | 12.4531104 | 12.1544134 | 10.0753763 | 10.6241986 | 9.99327753 | 10.5079787 |
| Ikzf2 | 0 | 0 | 0 | 11.2294386 | 10.0871853 | 10.8948866 |
| Il7R | 2.6756414 | 3.11340227 | 0 | 0 | 0 | 0 |
| Irf4 | 10.9460654 | 0 | 7.83866655 | 0 | 0 | 0 |
| Irf6 | 3.75002159 | 0 | 0 | 0 | 0 | 0 |
| Irf8 | 0 | 14.7096031 | 14.2888668 | 0 | 0 | 8.8628089 |
| Kdr | 0 | 0 | 0 | 0 | 0 | 0 |
| Kit | 0.24445292 | 0.50621599 | 11.0295653 | 12.5726203 | 12.3791378 | 12.0919625 |
| Klf1 | 5.84397562 | 0 | 0 | 0 | 8.46482083 | 0 |
| Klf12 | 8.05086964 | 0 | 0 | 0 | 0 | 0 |
| Ldb1 | 11.2686965 | 8.12945947 | 8.68054007 | 11.6527152 | 12.2469401 | 11.316521 |
| Lin28a | 1.96158082 | 6.99342123 | 5.60046956 | 8.55574345 | 6.63350297 | 8.68718725 |
| Lmo2 | 3.89774451 | 8.38732066 | 8.20469078 | 10.3169241 | 5.11243451 | 9.84586404 |
| Ly6a | 8.85142518 | 0 | 0 | 0 | 4.09411947 | 0 |
| Lyl1 | 0 | 9.75810271 | 0 | 0 | 0 | 9.50789901 |
| Mbd2 | 11.5068886 | 11.2014367 | 11.5257283 | 11.0058202 | 9.54315445 | 10.0659452 |
| Meis1 | 8.51879687 | 0 | 0 | 4.87021647 | 0 | 0 |
| Mllt3 | 1.72128743 | 0 | 0 | 0 | 0 | 1.98967093 |
| Mpl | 0 | 5.37493792 | 0 | 0 | 4.57579908 | 8.42884537 |
| Muc13 | 0 | 0 | 7.94365244 | 12.3910631 | 9.66287501 | 9.27532572 |
| Myb | 8.92481613 | 11.9021578 | 7.74778663 | 13.4608829 | 13.6082862 | 12.5062084 |
| Myc | 0 | 0 | 9.66579628 | 13.1468373 | 11.1237836 | 12.2368797 |
| Mycn | 0 | 0 | 0 | 0 | 4.71550783 | 0 |
| Ndn | 0 | 0 | 0 | 0 | 0 | 0 |
| Nfat5 | 1.68337396 | 6.42382445 | 8.11771068 | 8.50241858 | 8.29542914 | 6.81510443 |
| Nfia | 11.1966351 | 0.51538312 | 8.76871243 | 10.4414063 | 8.37541044 | 0 |
| Nfkb1 | 6.28053175 | 4.20047424 | 7.24237126 | 3.02501649 | 0 | 0 |
| Notch1 | 9.23968393 | 9.80621601 | 0 | 0 | 0 | 9.04276389 |
| Pax4 | 0 | 4.30341437 | 0 | 0 | 4.67280508 | 3.18275178 |
| Pax5 | 0 | 0 | 0 | 0 | 0 | 0 |
| Pax9 | 0 | 5.11348672 | 0 | 0 | 0 | 0 |
| Pbx1 | 0 | 0 | 0 | 0 | 0 | 2.85814132 |
| PIk3ca | 10.7501901 | 10.4597043 | 8.71137418 | 8.63082063 | 10.2150339 | 9.12110399 |
| PIk3R2 | 0 | 0 | 0 | 8.41565889 | 0 | 8.86044462 |
| Plag1 | 0 | 0 | 0 | 5.73253318 | 0 | 0 |
| Prf1 | 0 | 0 | 0 | 0 | 0 | 0 |
| Pten | 10.402978 | 10.7323361 | 8.45327824 | 9.15804062 | 8.02557223 | 9.55214218 |
| Rb1 | 11.5095723 | 10.3228048 | 11.0518462 | 8.80830469 | 10.975973 | 10.2070756 |
| Rora | 0 | 0 | 0 | 10.3525123 | 0 | 0 |
| Runx1 | 0 | 10.5448042 | 8.49404453 | 9.79896396 | 8.32589216 | 0 |
| Runx2 | 9.55408881 | 8.83337957 | 8.58263825 | 5.6671043 | 0 | 6.59981576 |
| Satb1 | 10.6618569 | 10.6425259 | 11.0333257 | 10.4623762 | 5.50666657 | 11.6829394 |
| Sdpr | 0 | 0 | 0 | 0 | 0 | 0 |
| Sell | 13.3986811 | 12.636786 | 11.8418847 | 12.1758077 | 8.32310492 | 10.6231619 |
| Sfpi1 | 10.755918 | 10.840172 | 10.234157 | 11.6285965 | 4.19803029 | 10.180779 |
| Slamf1 | 0 | 0 | 0 | 0 | 0 | 0 |
| Smarca4 | 12.4059967 | 12.3958203 | 10.7430601 | 12.6426923 | 9.78305678 | 11.6074547 |
| Sos1 | 1.96984274 | 7.60327488 | 8.55093991 | 7.00950203 | 7.97175828 | 7.25923732 |
| Stat1 | 0.57217994 | 4.66285063 | 8.17622822 | 7.02260834 | 5.60396427 | 6.85302887 |
| Stat3 | 12.1553826 | 10.5962174 | 10.1047053 | 10.4043949 | 10.6890265 | 11.1026336 |
| Stat4 | 11.2376366 | 0 | 0 | 8.1182282 | 0 | 6.31665833 |
| Stat6 | 10.4721199 | 9.57987162 | 0 | 10.8577127 | 8.31312981 | 8.9859846 |
| Suz12 | 8.50068008 | 11.8114564 | 10.0842116 | 11.4415014 | 8.88768825 | 10.3591033 |
| Tal1 | 0 | 0 | 7.35199805 | 7.41118762 | 0 | 3.34846603 |
| Tcf3 | 6.0690736 | 6.37460317 | 0 | 0 | 7.14327082 | 10.0950413 |
| Tcf4 | 13.9829509 | 13.2477205 | 11.1633078 | 10.5566707 | 10.2373849 | 11.9154368 |
| Tcf7 | 12.5483718 | 0 | 0 | 0 | 0 | 0 |
| Tek | 0 | 0 | 0 | 0 | 0 | 0 |
| Tfrc | 11.5310872 | 13.6794866 | 8.69647395 | 10.1124605 | 9.94594668 | 8.66198046 |
| Tgfb1 | 0 | 0 | 0 | 8.39098114 | 0 | 0 |
| Tgfb2 | 2.54299473 | 0 | 0 | 0 | 0 | 0 |
| Tgfb3 | 1.83073988 | 0 | 0 | 0 | 0 | 0 |
| Tnfrsf1a | 0 | 0 | 10.5575923 | 10.2288397 | 5.8586183 | 10.085531 |
| Tnfrsf1b | 5.27266462 | 0 | 0 | 10.3201112 | 0.95315427 | 0.80836534 |
| Tnfrsf21 | 0.70732573 | 0 | 6.05902828 | 7.64675137 | 0 | 7.86021375 |
| Tnfsf10 | 4.81322759 | 0 | 3.8552827 | 7.3711495 | 0 | 0 |
| Tnfsf12 | 0 | 0 | 4.39444523 | 0 | 0 | 0 |
| Tob1 | 3.38203155 | 0 | 7.3702815 | 8.22337837 | 0 | 5.83579043 |
| vWF | 0 | 0 | 0 | 0 | 0 | 0 |
| Zbtb20 | 6.49874585 | 8.98366904 | 7.76355827 | 7.5019406 | 9.51185133 | 9.03587558 |
| Zbtb38 | 6.31337663 | 0 | 8.66735889 | 8.88619321 | 8.85030113 | 7.99157356 |
| Zfp532 | 0 | 0 | 4.28968013 | 0 | 2.01705667 | 3.84180886 |
| Zfp612 | 5.14316607 | 0 | 1.45139554 | 6.82565849 | 0 | 4.40273428 |
| Zfpm1 | 0 | 0 | 0 | 0 | 0 | 0 |
| Zhx2 | 0 | 1.36199848 | 9.44707427 | 0 | 0 | 6.34007356 |

TABLE 6-5
Single cell expression data (reduced list)-Control

| Factor | CMP5 | CMP6 | CMP7 | CMP8 | CMP9 | CMP10 |
|---|---|---|---|---|---|---|
| Actb | 17.3394053 | 14.6706888 | 15.3006859 | 15.6706136 | 16.2161296 | 16.2031528 |
| Aebp2 | 7.48010576 | 4.52217501 | 4.85718391 | 6.22489648 | 6.15542349 | 6.65750054 |
| Ahr | 0 | 0 | 0 | 0 | 8.48248567 | 0 |
| Akt1 | 11.0295746 | 9.13888127 | 8.50202567 | 9.48522978 | 9.83325343 | 10.1423732 |
| Akt2 | 5.6982268 | 6.43649925 | 0 | 6.54782485 | 5.67097403 | 6.91885001 |
| Akt3 | 10.7535896 | 5.05597233 | 8.96329552 | 9.39938997 | 8.41514892 | 8.63112027 |
| APC | 0 | 0 | 5.85738488 | 0 | 0 | 8.00067699 |
| Bad | 0 | 0 | 0 | 0 | 0 | 0 |
| Bax | 10.7709938 | 7.60268797 | 9.74661453 | 9.46994606 | 10.0956302 | 9.66835081 |
| Bcl11a | 7.25102747 | 0 | 3.44256113 | 0 | 0 | 0 |
| Bcl11b | 0 | 0 | 0 | 0 | 0 | 0 |
| Bcl2 | 0 | 0 | 5.71221572 | 8.46600782 | 4.51709175 | 7.63420792 |
| Bcl211 | 0 | 0 | 8.642915 | 9.8449129 | 9.83242806 | 11.727409 |
| Bcl2111 | 4.94361446 | 6.96342995 | 0 | 8.82547082 | 7.49063229 | 0 |
| Bmi1 | 8.04079881 | 6.47044397 | 6.99413119 | 7.02301797 | 5.66629178 | 7.29852135 |
| Brd3 | 11.7497296 | 9.48652042 | 10.2279983 | 10.7336706 | 9.99622743 | 10.5589239 |
| Casp8 | 11.4458868 | 9.37414266 | 10.730553 | 11.5737089 | 10.042092 | 11.3341723 |
| Casp9 | 8.60157869 | 0.43486175 | 8.11116214 | 8.49830047 | 8.46979801 | 0 |
| Cbx2 | 8.14298572 | 5.42369511 | 0 | 2.02852747 | 6.14976979 | 0 |
| Cbx8 | 0 | 0 | 0 | 0 | 0 | 6.5352377 |
| Ccnc | 9.337732 | 0 | 0 | 0 | 8.74862406 | 8.05461177 |
| Ccnd1 | 12.3424395 | 0 | 0 | 5.08950715 | 10.3980334 | 9.67251383 |
| Ccne2 | 10.6836164 | 0 | 0 | 8.88454106 | 7.76036683 | 0 |
| CD34 | 13.0466336 | 0 | 10.0606452 | 11.7867314 | 8.70281995 | 11.9349176 |
| CD41 | 7.22234749 | 9.88958898 | 0 | 8.74031169 | 13.4959806 | 11.1372918 |
| CD48 | 12.0992452 | 10.568177 | 7.88392396 | 10.8210925 | 8.89620358 | 11.2734612 |
| CD52 | 11.0838001 | 0 | 5.49447739 | 8.00130213 | 7.2008291 | 7.95395412 |
| CD53 | 12.7670824 | 0 | 10.9959227 | 11.3777197 | 0 | 0 |
| CD55 | 0 | 8.42133148 | 0 | 0 | 9.29531826 | 0 |
| CD63 | 9.14519387 | 0 | 7.74259128 | 9.32290779 | 9.53162102 | 7.281967 |
| CD9 | 0 | 0 | 0 | 0 | 9.68777068 | 0 |
| Cdc42 | 14.6585333 | 12.6841565 | 13.4268211 | 13.5192656 | 13.4441459 | 13.1256535 |
| Cdk1 | 10.9097239 | 6.60224216 | 0 | 9.60826336 | 9.2659687 | 11.8683968 |
| Cdk4 | 12.2911932 | 9.86090165 | 7.8025631 | 11.0577815 | 11.3768742 | 11.0385295 |
| Cdkn2b | 0 | 0 | 0 | 0 | 0 | 0 |
| Cebpa | 12.8418824 | 0 | 10.2324455 | 13.6075773 | 8.81482957 | 11.9755884 |
| Csf1r | 11.0511238 | 0 | 0 | 10.585565 | 7.27360003 | 3.88021025 |
| Ctnnb1 | 8.35670072 | 4.81362741 | 5.97188813 | 5.22508782 | 8.07136491 | 8.28703889 |
| Cycs | 14.5377046 | 11.2691463 | 10.1789357 | 13.0405966 | 12.4297442 | 13.3283287 |
| Dach1 | 4.97803655 | 4.14474045 | 10.5451334 | 8.59226416 | 11.9267309 | 13.5465833 |
| Dnmt1 | 12.8726368 | 10.4919004 | 0 | 12.5203344 | 12.4834927 | 12.7064491 |
| Dnmt3a | 11.0265538 | 11.1062288 | 10.9186344 | 5.45624458 | 10.3948879 | 8.98758434 |
| Dnmt3b | 10.5790239 | 0 | 8.38337161 | 9.97828774 | 10.4507647 | 10.9212224 |
| Dtx1 | 4.3790403 | 0 | 0 | 0.78348056 | 4.24129098 | 0 |
| Dtx4 | 11.1502546 | 0 | 10.8469873 | 8.96806057 | 8.43544431 | 0 |
| Ebf1 | 0 | 0 | 0 | 0 | 0 | 0 |
| Ep300 | 10.4632229 | 10.6518923 | 9.84642833 | 10.2654483 | 11.2467128 | 10.6061578 |
| Epor | 3.12221538 | 5.0756706 | 5.30043509 | 0.65533034 | 5.10260705 | 2.33815245 |
| Erg | 10.3534511 | 0 | 10.8266427 | 10.3592454 | 10.8159451 | 10.2449054 |
| Esr1 | 10.4969031 | 0 | 7.69419665 | 9.81964633 | 0 | 10.7394097 |
| ETS1 | 0 | 0 | 0 | 0 | 8.4833129 | 0 |
| ETS2 | 4.07083276 | 8.45916169 | 8.45527663 | 0 | 7.1341973 | 0.7101611 |
| Etv3 | 4.43481527 | 0 | 0 | 6.56778632 | 4.52654183 | 4.69321163 |
| Etv6 | 11.1448929 | 9.69394925 | 11.1261285 | 10.0656969 | 11.7161763 | 11.8183036 |
| Ezh2 | 10.8670738 | 7.48291356 | 6.20161136 | 8.65707232 | 9.49516932 | 9.8783733 |
| Fas | 0 | 0 | 0 | 0 | 6.62884488 | 0 |
| Fcgr2b | 9.85267441 | 0 | 0 | 8.23013247 | 0 | 0 |
| Fcgr3 | 0 | 0 | 8.119839 | 6.61788198 | 0 | 0 |
| Fli1 | 11.1890149 | 7.33814185 | 10.2757687 | 12.4967795 | 12.0912236 | 10.2473636 |
| Flt3 | 12.6574132 | 0 | 8.78397217 | 8.19832375 | 0 | 0 |
| Fosl1 | 8.40640045 | 0 | 0 | 0 | 0 | 0 |
| Foxo1 | 10.3981463 | 8.56491822 | 10.2557995 | 8.32166089 | 10.0603533 | 10.0759643 |
| Foxo3 | 9.39347931 | 8.64471911 | 10.6380669 | 10.7062816 | 10.0359107 | 9.78384345 |
| Gapdh | 13.8965059 | 9.81728739 | 8.9549559 | 10.5129808 | 11.6006197 | 11.7863478 |
| Gata1 | 0 | 11.2237171 | 8.02113847 | 0 | 9.99443513 | 10.5689067 |
| Gata2 | 2.95452348 | 2.89363096 | 3.76227155 | 4.70253038 | 7.1084613 | 7.11132825 |
| Gata3 | 0 | 0 | 7.93855591 | 0 | 5.10350469 | 2.65446248 |
| Gfi1 | 0 | 0 | 0 | 6.53413949 | 0 | 0 |
| Gfi1b | 0 | 9.24282738 | 8.39289491 | 9.64648209 | 0 | 0 |
| Hes5 | 0 | 1.45446472 | 0 | 0 | 0 | 0 |
| Hey1 | 0 | 0 | 0 | 0 | 0 | 0 |
| Hlf | 10.1384281 | 0 | 12.7210851 | 9.7255738 | 0 | 7.72908307 |
| Id2 | 0 | 0 | 0 | 0 | 0 | 0 |
| Ifi203 | 12.0764195 | 0 | 11.9267051 | 9.88952822 | 9.82976425 | 11.7604599 |
| Ifi205 | 0 | 0 | 0 | 0 | 0 | 0 |
| Ifitm1 | 10.026959 | 13.4455145 | 10.1142515 | 9.62949447 | 7.52584164 | 9.35647384 |
| Ikzf1 | 11.1162893 | 10.1776721 | 9.8444204 | 9.56063417 | 9.76312629 | 10.2802226 |
| Ikzf2 | 9.29677615 | 9.26597898 | 10.0113973 | 10.6548835 | 0 | 11.7095844 |
| Il7R | 0 | 0 | 0 | 0 | 0 | 0 |
| Irf4 | 0 | 0 | 0 | 0 | 0 | 0 |
| Irf6 | 0 | 4.07511467 | 4.21960052 | 0 | 3.38207598 | 1.77623393 |
| Irf8 | 11.496976 | 0.64529505 | 8.3919475 | 9.72740536 | 0 | 5.4029575 |
| Kdr | 0 | 0 | 0 | 0 | 0 | 0 |
| Kit | 12.5171017 | 12.5279914 | 12.8127026 | 13.3103212 | 12.0370385 | 12.3472302 |
| Klf1 | 0 | 10.8766523 | 0 | 0 | 7.07393535 | 0 |
| Klf12 | 0 | 0 | 0 | 0 | 0 | 0 |
| Ldb1 | 12.6976636 | 12.9835097 | 12.2468903 | 12.365463 | 12.0631399 | 10.863801 |
| Lin28a | 6.74728963 | 7.35105581 | 6.84975068 | 6.51455602 | 4.68753784 | 6.94552367 |
| Lmo2 | 10.5379436 | 9.34407841 | 10.2403324 | 11.0343922 | 11.009923 | 9.20928647 |
| Ly6a | 0 | 0 | 0 | 0 | 0 | 0 |
| Lyl1 | 8.51165848 | 0 | 7.37416084 | 9.52238028 | 0 | 8.2113635 |
| Mbd2 | 10.784295 | 10.4108785 | 10.1955821 | 10.6851427 | 10.63494 | 9.7729213 |
| Meis1 | 1.70692163 | 0 | 8.17244297 | 7.73150896 | 9.6859662 | 9.65905238 |
| Mllt3 | 0 | 0 | 0 | 0 | 0 | 0 |
| Mpl | 0 | 0 | 8.32138383 | 9.02983537 | 10.6949276 | 9.03531377 |
| Muc13 | 10.0268838 | 10.6859087 | 10.408149 | 10.9764924 | 10.4182397 | 10.4076086 |
| Myb | 13.3352034 | 13.5300503 | 12.2422918 | 13.8875021 | 12.5291358 | 12.9438126 |
| Myc | 14.3568801 | 13.271873 | 11.7486234 | 13.7517564 | 13.3585202 | 13.2342566 |
| Mycn | 0 | 3.51516181 | 8.22715916 | 0 | 12.5856289 | 9.69546069 |
| Ndn | 0 | 0 | 0 | 0 | 0 | 0 |
| Nfat5 | 6.28186815 | 5.14468333 | 9.22002325 | 8.30051998 | 6.00790584 | 7.26449937 |
| Nfia | 7.45568183 | 8.69437239 | 0 | 9.99729448 | 0 | 0 |
| Nfkb1 | 4.00335392 | 1.98855259 | 4.45405858 | 4.48909452 | 5.48703027 | 4.4024728 |
| Notch1 | 8.69453625 | 0 | 7.51819143 | 9.54735802 | 0 | 0 |
| Pax4 | 0 | 0.4697586 | 0 | 0 | 1.82857332 | 0 |
| Pax5 | 0 | 0 | 0 | 0 | 0 | 0 |
| Pax9 | 1.81855969 | 0 | 0 | 0 | 0 | 6.9598383 |
| Pbx1 | 0 | 4.33008847 | 0 | 0 | 0 | 0 |
| PIk3ca | 11.4126663 | 7.92679365 | 10.1322248 | 10.2563679 | 10.571161 | 10.2438679 |
| PIk3R2 | 10.1509326 | 7.23727926 | 10.6194334 | 0 | 10.0876344 | 8.17463706 |
| Plag1 | 0 | 8.268993 | 0 | 0 | 8.61635192 | 8.90930204 |
| Prf1 | 0 | 0 | 0 | 0 | 0 | 0 |
| Pten | 10.0980102 | 8.44584924 | 10.8389704 | 10.0450831 | 9.96600275 | 9.11441299 |
| Rb1 | 10.5049014 | 11.035184 | 10.2686739 | 8.28260838 | 11.2325685 | 0 |
| Rora | 0 | 0 | 9.40803685 | 0 | 0 | 0 |
| Runx1 | 9.80351196 | 9.89394529 | 11.2310772 | 10.8511201 | 9.61241397 | 11.260184 |
| Runx2 | 7.14600662 | 0 | 6.60312795 | 6.31525159 | 5.12629061 | 5.98996282 |
| Satb1 | 11.6190523 | 0 | 10.5185268 | 9.31688989 | 0 | 0 |
| Sdpr | 0 | 0 | 0 | 0 | 3.41819471 | 0 |
| Sell | 13.4721541 | 2.89807481 | 11.2017393 | 12.147405 | 7.52145725 | 11.8940425 |
| Sfpi1 | 12.1814824 | 0 | 10.3051236 | 11.135862 | 10.6759176 | 8.61401742 |
| Slamf1 | 0 | 0 | 0 | 0 | 8.27576355 | 0 |
| Smarca4 | 13.4159099 | 12.6986337 | 11.0603738 | 12.4208763 | 11.5131011 | 13.1672711 |
| Sos1 | 7.05920683 | 6.93067259 | 7.46342294 | 7.99375888 | 8.94290202 | 8.2090476 |
| Stat1 | 6.96525561 | 3.0714838 | 3.32406997 | 6.73484676 | 2.55117066 | 1.90884457 |
| Stat3 | 10.2805664 | 7.3966824 | 11.052227 | 11.4922447 | 9.33437336 | 11.3081762 |
| Stat4 | 7.45961139 | 0 | 8.01611823 | 6.16856977 | 7.27293514 | 9.2165467 |
| Stat6 | 9.84695626 | 7.3258474 | 10.0351652 | 9.04651696 | 9.68468703 | 9.93759651 |
| Suz12 | 12.160067 | 9.36880984 | 10.4227735 | 11.2065549 | 11.719744 | 11.4025496 |
| Tal1 | 4.01061915 | 6.6880475 | 7.39995658 | 0 | 0 | 0 |
| Tcf3 | 9.28106881 | 8.46463489 | 10.0783131 | 6.78607403 | 8.04893309 | 7.02457762 |
| Tcf4 | 11.9822362 | 10.7280242 | 10.8947009 | 10.4060663 | 9.61927383 | 10.7021269 |
| Tcf7 | 5.23267198 | 1.68626678 | 0 | 0 | 2.66766182 | 2.23952747 |
| Tek | 0 | 0 | 0 | 0 | 0 | 8.15055552 |
| Tfrc | 11.5315055 | 10.3078535 | 8.70556098 | 0 | 10.6282683 | 10.1053058 |
| Tgfb1 | 8.61614955 | 5.35612843 | 0 | 0 | 6.65768412 | 6.16568389 |
| Tgfb2 | 0 | 0 | 0 | 0 | 0 | 0 |
| Tgfb3 | 8.18570265 | 0 | 6.23265555 | 6.9879955 | 0 | 6.47320472 |
| Tnfrsf1a | 11.3960482 | 9.71137069 | 10.5553381 | 10.0882949 | 8.80578171 | 9.01361307 |
| Tnfrsf1b | 9.21806977 | 0 | 9.61506083 | 8.80892599 | 9.64596728 | 4.62484099 |
| Tnfrsf21 | 7.08978321 | 5.63889855 | 3.52361608 | 5.13475364 | 7.18706943 | 6.49011462 |
| Tnfsf10 | 0 | 0 | 0 | 7.44776059 | 0 | 4.80467952 |
| Tnfsf12 | 0 | 0 | 4.92147767 | 0 | 0 | 6.45276939 |
| Tob1 | 0 | 0 | 4.87096526 | 0 | 0 | 0 |
| vWF | 0 | 0 | 0 | 0 | 0.92959921 | 0 |
| Zbtb20 | 8.91468776 | 7.47378037 | 8.65801097 | 6.07085525 | 7.77205018 | 9.83080899 |
| Zbtb38 | 7.61532556 | 8.16188767 | 7.21002151 | 9.37139278 | 9.52940602 | 7.19300308 |
| Zfp532 | 0 | 4.20413936 | 0 | 0 | 0 | 2.33025492 |
| Zfp612 | 6.36251023 | 0 | 0 | 5.89338537 | 5.72389563 | 0 |
| Zfpm1 | 0 | 7.38814478 | 0 | 6.75057183 | 4.81492174 | 0 |
| Zhx2 | 0 | 10.0153129 | 0 | 10.0672844 | 0 | 0 |

TABLE 6-6
Single cell expression data (reduced list)-Control

| Factor | GMP1 | GMP2 | GMP3 | GMP4 | GMP5 | GMP6 |
|---|---|---|---|---|---|---|
| Actb | 17.1489215 | 17.1987952 | 17.0261935 | 17.386841 | 16.8304269 | 16.7489209 |
| Aebp2 | 7.38412472 | 7.37000886 | 7.67068492 | 8.3165713 | 5.4136843 | 7.57713129 |
| Ahr | 0 | 0 | 0 | 0 | 8.2586416 | 2.48178389 |
| Akt1 | 11.235626 | 11.370018 | 11.2228314 | 11.4580108 | 9.35433585 | 11.3917982 |
| Akt2 | 0 | 5.65369871 | 6.60168541 | 7.30834154 | 7.09194507 | 7.27954511 |
| Akt3 | 9.2040554 | 6.42589774 | 7.76683642 | 10.3335 | 0 | 0 |
| APC | 0 | 0 | 10.3835517 | 0 | 8.371236 | 0 |
| Bad | 0 | 0 | 0 | 0 | 0 | 0 |
| Bax | 12.3982935 | 11.548933 | 11.7457261 | 12.5304908 | 9.63819013 | 9.58757022 |
| Bcl11a | 0 | 4.8496745 | 5.5277101 | 0 | 0 | 0 |
| Bcl11b | 2.47388586 | 0 | 0 | 3.3676317 | 4.51519907 | 0 |
| Bcl2 | 8.67205883 | 4.74052395 | 7.4793676 | 9.81638057 | 0 | 0 |
| Bcl211 | 11.2985207 | 10.9107736 | 8.31831953 | 10.0601684 | 7.45200039 | 0 |
| Bcl2111 | 9.91590871 | 8.18472841 | 7.91574582 | 8.84722554 | 10.1748095 | 6.43500489 |
| Bmi1 | 7.65085777 | 4.83187475 | 9.02271832 | 6.18509638 | 7.09454308 | 7.56761362 |
| Brd3 | 12.2200241 | 8.5222524 | 12.5897181 | 12.3613327 | 12.0766338 | 11.4340477 |
| Casp8 | 11.9935864 | 12.4728177 | 11.2081299 | 11.7931878 | 10.6330727 | 9.95275872 |
| Casp9 | 9.85784236 | 9.2795417 | 10.4608042 | 9.30079864 | 8.68972348 | 8.67710004 |
| Cbx2 | 8.13468181 | 6.26338723 | 4.15904155 | 2.80402938 | 0 | 4.90815454 |
| Cbx8 | 0 | 0 | 0 | 0 | 0 | 0 |
| Ccnc | 6.54457096 | 7.80869339 | 10.2612515 | 10.5944974 | 9.89068237 | 8.39273481 |
| Ccnd1 | 8.58525018 | 9.07320206 | 0.44602581 | 11.6985658 | 0 | 8.4714389 |
| Ccne2 | 10.2847235 | 10.3613222 | 10.2263111 | 7.68162663 | 7.00126105 | 7.38398862 |
| CD34 | 9.76737788 | 11.3493653 | 12.3762338 | 12.665751 | 0.7308249 | 0 |
| CD41 | 0 | 0 | 9.92285908 | 10.1379171 | 0 | 0 |
| CD48 | 11.1755703 | 12.3720324 | 11.2216769 | 13.1172131 | 8.98467946 | 11.1712268 |
| CD52 | 12.214887 | 11.4843836 | 6.92750614 | 10.055469 | 9.88050006 | 9.66769309 |
| CD53 | 13.734581 | 12.9470142 | 11.5566919 | 12.0795346 | 11.4796107 | 11.6332867 |
| CD55 | 0 | 0 | 0 | 0 | 0 | 1.98400237 |
| CD63 | 5.83669083 | 10.6791061 | 11.1660619 | 9.5002936 | 11.8417986 | 11.5674632 |
| CD9 | 7.33502006 | 0 | 10.0478265 | 0 | 9.8535396 | 9.37192294 |
| Cdc42 | 15.071603 | 14.9063997 | 14.4251672 | 15.2700451 | 14.1058059 | 14.2812027 |
| Cdk1 | 11.1089539 | 12.565398 | 10.0640308 | 12.9451584 | 8.92252913 | 10.3979323 |
| Cdk4 | 12.1492532 | 12.2049096 | 11.3481552 | 12.5805625 | 10.3340466 | 10.1996484 |
| Cdkn2b | 0 | 1.8448054 | 0 | 0 | 0 | 0 |
| Cebpa | 13.5582841 | 13.0751849 | 14.1307094 | 14.8662046 | 13.3279428 | 12.940603 |
| Csf1r | 13.2965977 | 9.82859309 | 9.7227165 | 12.1147466 | 7.15970464 | 7.14539069 |
| Ctnnb1 | 9.16188305 | 7.52545352 | 8.60919966 | 9.20385918 | 8.67653144 | 9.17983079 |
| Cycs | 14.5117323 | 15.5509006 | 14.3926146 | 14.8500674 | 13.3320521 | 13.6650347 |
| Dach1 | 10.9910945 | 8.44938041 | 12.3883714 | 8.79080043 | 10.4536266 | 10.6691965 |
| Dnmt1 | 12.9020312 | 12.4369612 | 12.7558873 | 12.902768 | 11.6602754 | 11.0715158 |
| Dnmt3a | 10.7289813 | 6.56627584 | 10.702069 | 11.2807594 | 10.0406974 | 10.0659832 |
| Dnmt3b | 9.58857441 | 6.44688601 | 10.7118482 | 10.5910128 | 8.18039351 | 7.21703334 |
| Dtx1 | 3.0913916 | 0 | 3.91641931 | 0 | 0 | 0 |
| Dtx4 | 10.1882254 | 11.1715529 | 12.6766112 | 13.3330567 | 12.3246264 | 12.2398755 |
| Ebf1 | 0 | 0 | 0 | 0 | 0 | 6.35563108 |
| Ep300 | 11.0646985 | 5.51844512 | 10.4585713 | 10.8818586 | 10.7818993 | 10.2687707 |
| Epor | 4.1948605 | 5.82587694 | 4.04624715 | 4.16263046 | 4.31309197 | 5.7777581 |
| Erg | 10.0476497 | 10.8998172 | 8.31856172 | 10.7787749 | 8.41282235 | 8.00315491 |
| Esr1 | 0 | 9.61295568 | 7.43332756 | 11.6298664 | 9.26139595 | 0 |
| ETS1 | 0 | 8.49664543 | 0 | 0 | 11.774333 | 10.8678821 |
| ETS2 | 0 | 7.04070704 | 8.18875575 | 0.30773145 | 9.76422043 | 0 |
| Etv3 | 0 | 5.70625189 | 4.29581374 | 5.43089153 | 4.8703617 | 1.40350183 |
| Etv6 | 12.0523052 | 11.0382089 | 9.74143581 | 13.0923382 | 0 | 9.61119192 |
| Ezh2 | 11.652838 | 11.5860694 | 11.1993861 | 11.4872376 | 10.1109725 | 10.4391363 |
| Fas | 0 | 0 | 0 | 0 | 0 | 8.72358173 |
| Fcgr2b | 9.19136771 | 8.72106918 | 9.14865833 | 8.70635442 | 10.0101786 | 7.27372444 |
| Fcgr3 | 10.5154928 | 11.1483415 | 9.97180324 | 10.3691572 | 10.0558965 | 10.006567 |
| Fli1 | 12.1113098 | 10.2964886 | 11.1111683 | 13.0309888 | 12.5529343 | 13.2435265 |
| Flt3 | 0 | 0 | 0 | 8.20154666 | 0 | 0 |
| Fosl1 | 9.14818795 | 0 | 0 | 0 | 0 | 8.95584384 |
| Foxo1 | 10.6678286 | 7.11027738 | 10.5639142 | 11.4065349 | 7.68627588 | 8.03189028 |
| Foxo3 | 8.6581534 | 8.83051249 | 9.05928824 | 10.1872797 | 8.17891127 | 9.65874783 |
| Gapdh | 14.5697489 | 14.8364814 | 14.4841585 | 15.2948511 | 12.4951942 | 11.7288864 |
| Gata1 | 2.68117413 | 5.3286228 | 2.40405255 | 0 | 6.20074437 | 0 |
| Gata2 | 0 | 0 | 1.18893452 | 5.03280609 | 0 | 0 |
| Gata3 | 0 | 0 | 0 | 0 | 0 | 0 |
| Gfi1 | 0 | 9.95120128 | 10.4607555 | 10.4250456 | 10.2403166 | 9.63774464 |
| Gfi1b | 0 | 0 | 0 | 0 | 0 | 0 |
| Hes5 | 0 | 0 | 0 | 0 | 0 | 0 |
| Hey1 | 0 | 0 | 0 | 0 | 0 | 0 |
| Hlf | 0 | 0 | 0 | 8.36237805 | 0 | 0 |
| Id2 | 0 | 0 | 0 | 0 | 8.92503527 | 6.47603665 |
| Ifi203 | 12.4820599 | 10.2059101 | 9.66114357 | 10.9751352 | 0 | 8.43723516 |
| Ifi205 | 0 | 4.02559453 | 0 | 0 | 0 | 0 |
| Ifitm1 | 0 | 7.59695531 | 9.85532823 | 0 | 7.57013634 | 0 |
| Ikzf1 | 12.0462915 | 11.0027006 | 10.1806326 | 12.6066347 | 10.1787075 | 9.1812643 |
| Ikzf2 | 8.42131399 | 6.66467431 | 9.37167983 | 0 | 0 | 0 |
| Il7R | 0 | 0 | 0 | 0 | 0 | 0 |
| Irf4 | 0 | 0 | 0 | 0 | 0 | 0 |
| Irf6 | 0 | 2.58748455 | 0 | 0 | 3.59687181 | 0 |
| Irf8 | 13.8990229 | 12.7012696 | 0.47691932 | 14.1636759 | 1.98646599 | 0 |
| Kdr | 0 | 0 | 0 | 0 | 0 | 0 |
| Kit | 11.4828646 | 10.9088944 | 12.3859747 | 13.5844173 | 11.2619077 | 12.5333324 |
| Klf1 | 0 | 0 | 1.53786674 | 0 | 1.35001333 | 0 |
| Klf12 | 0 | 0 | 0 | 0 | 0 | 0 |
| Ldb1 | 11.6022208 | 12.5920203 | 10.6653308 | 12.5336097 | 11.495488 | 12.3066988 |
| Lin28a | 7.90392609 | 3.12320396 | 6.33237234 | 1.79305028 | 6.59839184 | 7.06652167 |
| Lmo2 | 9.87792981 | 9.91993508 | 11.2848458 | 11.9924048 | 10.0290151 | 10.1483392 |
| Ly6a | 7.86019166 | 0 | 0 | 7.73891186 | 7.84163194 | 0 |
| Lyl1 | 0 | 8.217674 | 9.1254904 | 9.44826214 | 8.82368626 | 8.22225726 |
| Mbd2 | 12.0598914 | 12.2588426 | 11.112991 | 11.5901371 | 9.15035566 | 11.3382915 |
| Meis1 | 0 | 0 | 4.25525076 | 7.0666261 | 0 | 0 |
| Mllt3 | 0 | 0 | 0 | 0 | 0 | 0 |
| Mpl | 0 | 0 | 0 | 0 | 0 | 0 |
| Muc13 | 6.21902111 | 6.09924564 | 10.3554653 | 9.73058449 | 0 | 9.36857432 |
| Myb | 12.6635861 | 11.8365941 | 14.2028029 | 14.5090875 | 13.301967 | 14.4078534 |
| Myc | 9.1352006 | 14.8322048 | 14.0818035 | 15.1689656 | 10.5951842 | 12.5380787 |
| Mycn | 5.64989123 | 0 | 0 | 0 | 0 | 0 |
| Ndn | 0 | 0 | 0 | 0 | 0 | 0 |
| Nfat5 | 10.8366496 | 3.68452734 | 8.92920727 | 10.0449498 | 9.57236112 | 7.70240307 |
| Nfia | 8.66284129 | 9.64290212 | 8.28353384 | 0 | 0 | 0 |
| Nfkb1 | 4.1409895 | 1.10789555 | 6.0665323 | 2.33679964 | 4.16758728 | 3.4944722 |
| Notch1 | 9.67689195 | 7.78055521 | 10.5333446 | 10.0774827 | 10.2196335 | 10.5378767 |
| Pax4 | 0 | 2.69956228 | 4.5651786 | 0 | 0 | 0 |
| Pax5 | 0 | 0 | 0 | 0 | 0 | 0 |
| Pax9 | 0 | 0 | 0 | 0 | 0 | 0 |
| Pbx1 | 0 | 0 | 0 | 1.84774739 | 0 | 0 |
| PIk3ca | 11.6583177 | 9.35235227 | 10.3476041 | 11.0004673 | 9.86525632 | 11.9782697 |
| PIk3R2 | 10.3431352 | 9.62249368 | 9.03318404 | 11.2354698 | 0 | 9.21073238 |
| Plag1 | 2.1446229 | 0 | 0 | 0 | 0 | 0 |
| Prf1 | 0 | 0 | 0 | 0 | 0 | 0 |
| Pten | 11.4197765 | 9.13702301 | 10.7392588 | 12.0713175 | 10.7961825 | 11.0569877 |
| Rb1 | 12.3671936 | 9.29319202 | 10.4219806 | 10.1129328 | 11.381463 | 10.889451 |
| Rora | 0 | 0 | 0 | 0 | 0 | 7.95341913 |
| Runx1 | 13.783041 | 11.1039612 | 11.2727924 | 14.2307475 | 10.5352512 | 12.0416809 |
| Runx2 | 5.65817302 | 5.03497789 | 4.41480127 | 5.28240362 | 5.90471616 | 6.86059385 |
| Satb1 | 9.63218514 | 0 | 9.35749111 | 10.3868222 | 10.9890151 | 9.27731882 |
| Sdpr | 0 | 0 | 0 | 0 | 0 | 0 |
| Sell | 13.346662 | 14.2717617 | 11.4404307 | 12.6975062 | 10.7600258 | 10.916911 |
| Sfpi1 | 12.9675055 | 11.9210703 | 12.4452889 | 13.2408628 | 11.6645721 | 12.6354578 |
| Slamf1 | 0 | 0 | 0 | 0 | 0 | 0 |
| Smarca4 | 12.6576943 | 11.1882941 | 13.3524008 | 13.9464355 | 12.5556067 | 12.548269 |
| Sos1 | 8.75240526 | 4.47302434 | 9.76938074 | 9.21626024 | 5.77526698 | 8.46060551 |
| Stat1 | 7.83159291 | 5.19471875 | 1.94245366 | 3.19107626 | 3.69538692 | 4.99541136 |
| Stat3 | 9.94864616 | 8.03134798 | 12.2126573 | 12.2361408 | 12.6530163 | 11.4027843 |
| Stat4 | 7.30783486 | 6.44025276 | 8.04438756 | 6.41767238 | 7.74175516 | 9.24847993 |
| Stat6 | 11.4183952 | 7.63189419 | 11.402629 | 11.427093 | 11.1296225 | 11.0864028 |
| Suz12 | 12.0645852 | 9.97123248 | 12.3070014 | 12.586926 | 11.1205885 | 11.9182639 |
| Tal1 | 0 | 0 | 3.6852286 | 7.53257554 | 7.0164346 | 6.40585349 |
| Tcf3 | 8.17529451 | 8.44265648 | 0.46728578 | 7.69609118 | 0.32105529 | 7.98262856 |
| Tcf4 | 10.7240061 | 10.8374419 | 11.2234939 | 12.5413021 | 9.18774076 | 9.58716005 |
| Tcf7 | 0 | 0 | 0 | 0 | 0 | 0 |
| Tek | 0 | 0 | 0 | 0 | 0 | 0 |
| Tfrc | 11.0749821 | 12.561574 | 12.1280736 | 13.583871 | 11.1008997 | 11.8397881 |
| Tgfb1 | 6.27914163 | 9.26600463 | 9.08857843 | 9.47356083 | 4.49109661 | 0 |
| Tgfb2 | 3.56183374 | 0 | 0 | 0 | 0 | 0 |
| Tgfb3 | 0 | 0 | 5.7011227 | 0.46458839 | 8.34339661 | 0 |
| Tnfrsf1a | 12.297425 | 11.7846035 | 11.7957289 | 13.0383546 | 11.8629069 | 12.0139251 |
| Tnfrsf1b | 12.4247113 | 8.90867624 | 12.1885403 | 11.8433223 | 10.5206013 | 10.2570003 |
| Tnfrsf21 | 7.44931949 | 6.46752449 | 7.14549464 | 7.31352162 | 7.9695614 | 7.76578158 |
| Tnfsf10 | 0 | 1.68498558 | 6.44830699 | 5.74757111 | 6.17892222 | 0 |
| Tnfsf12 | 0 | 0 | 0 | 0 | 0 | 0 |
| Tob1 | 0 | 0 | 0 | 6.10049513 | 0 | 0 |
| vWF | 0 | 0 | 0 | 0 | 0 | 0 |
| Zbtb20 | 0 | 0 | 0 | 8.30629918 | 0 | 0 |
| Zbtb38 | 8.27537196 | 8.75347218 | 10.5074098 | 10.1488632 | 9.05482607 | 10.0593391 |
| Zfp532 | 0 | 1.56494117 | 0 | 0 | 0 | 2.30677569 |
| Zfp612 | 0 | 3.91554231 | 1.00265837 | 6.21466929 | 7.67481421 | 0.57219649 |
| Zfpm1 | 0 | 0 | 0 | 5.38371259 | 0 | 0 |
| Zhx2 | 0 | 6.85838682 | 0 | 3.16109771 | 8.51542476 | 0 |

TABLE 6-7
Single cell expression data (reduced list)-Control

| Factor | GMP7 | GMP8 | GMP9 | GMP10 | HSC1 | HSC2 |
|---|---|---|---|---|---|---|
| Actb | 16.9514796 | 17.399739 | 17.2637454 | 16.9850638 | 14.2167236 | 14.6194148 |
| Aebp2 | 7.35505455 | 4.38592355 | 5.1807596 | 7.51562781 | 2.42975426 | 4.97605754 |
| Ahr | 0 | 8.88485487 | 10.3510122 | 0 | 0 | 0 |
| Akt1 | 12.2492506 | 10.7788814 | 9.09878888 | 11.5407814 | 8.96092519 | 8.92881088 |
| Akt2 | 7.07125847 | 6.57841965 | 5.05613909 | 8.09120983 | 0 | 5.44823903 |
| Akt3 | 9.84112573 | 10.6234887 | 8.79800603 | 10.3335926 | 0 | 9.31021549 |
| APC | 0 | 0 | 8.16762557 | 8.43918267 | 0 | 0 |
| Bad | 0 | 0 | 0 | 0 | 0 | 0 |
| Bax | 11.0109809 | 11.0453066 | 9.34116544 | 11.9634436 | 7.34390449 | 8.34746535 |
| Bcl11a | 0 | 9.41212409 | 0 | 0 | 8.75277008 | 0 |
| Bcl11b | 0 | 0 | 0 | 1.88740222 | 0 | 0 |
| Bcl2 | 0 | 0 | 0 | 8.52796043 | 5.87135064 | 0 |
| Bcl211 | 9.44244435 | 10.1472452 | 0 | 11.1322976 | 8.66094346 | 9.94832245 |
| Bcl2111 | 10.1673298 | 0 | 0 | 0 | 0 | 8.69198824 |
| Bmi1 | 8.07353481 | 7.72482902 | 4.98516188 | 8.47434036 | 6.82657462 | 7.46085956 |
| Brd3 | 12.6394847 | 11.2028078 | 7.11480939 | 11.8951694 | 9.33404025 | 8.63333449 |
| Casp8 | 11.8613695 | 9.99564976 | 9.21248114 | 11.5898934 | 0 | 8.6154989 |
| Casp9 | 8.59054116 | 8.91150088 | 8.46508701 | 8.65641125 | 8.2106278 | 0 |
| Cbx2 | 4.51981855 | 0 | 0 | 0 | 0 | 0 |
| Cbx8 | 7.59923933 | 0 | 0 | 5.95563266 | 4.01892229 | 0 |
| Ccnc | 5.81056153 | 1.75012419 | 6.70114967 | 7.82322872 | 0 | 7.8085882 |
| Ccnd1 | 11.5505776 | 0 | 10.1157016 | 9.71290948 | 0 | 8.62150748 |
| Ccne2 | 11.303028 | 9.04842269 | 0 | 9.50031357 | 0 | 4.39863781 |
| CD34 | 12.2237971 | 0 | 8.89631259 | 13.6407341 | 9.50379181 | 9.06540049 |
| CD41 | 0 | 0 | 0 | 0 | 0 | 0 |
| CD48 | 11.4659003 | 9.71355517 | 10.4133748 | 11.4910927 | 0 | 0 |
| CD52 | 9.60985547 | 9.93196311 | 12.5022437 | 10.7028269 | 0 | 0 |
| CD53 | 12.1131339 | 12.7875274 | 11.5957042 | 12.2029543 | 0 | 0 |
| CD55 | 0 | 0 | 0 | 0 | 6.89471557 | 7.36408685 |
| CD63 | 8.93841954 | 12.146554 | 0 | 5.48306679 | 9.19375582 | 7.65368115 |
| CD9 | 0 | 10.1324772 | 7.67704046 | 0 | 7.8387743 | 0 |
| Cdc42 | 14.4664142 | 14.2907989 | 14.0122499 | 15.0649621 | 11.9634665 | 12.0459978 |
| Cdk1 | 11.3777802 | 8.11959637 | 0 | 12.7269855 | 0 | 0 |
| Cdk4 | 12.784903 | 10.8753402 | 6.80400834 | 12.6121689 | 9.62020787 | 8.49447754 |
| Cdkn2b | 0 | 0.00701553 | 0 | 0 | 0 | 0 |
| Cebpa | 13.8746339 | 13.8824666 | 0 | 14.641417 | 0 | 8.06551113 |
| Csf1r | 11.5330216 | 3.88795501 | 7.38801037 | 12.5028245 | 0.17278247 | 0 |
| Ctnnb1 | 8.77284547 | 8.15585683 | 7.63240721 | 9.49085314 | 7.84991528 | 6.63261919 |
| Cycs | 14.9720652 | 13.8929845 | 11.7488184 | 14.6315404 | 9.69074953 | 9.01652869 |
| Dach1 | 10.0139282 | 11.094158 | 0 | 0 | 0 | 9.34452255 |
| Dnmt1 | 13.8203577 | 13.062377 | 8.93180003 | 12.6151647 | 8.13040287 | 8.73259462 |
| Dnmt3a | 11.5907989 | 10.5082482 | 8.16704073 | 12.2259286 | 0 | 9.03600947 |
| Dnmt3b | 10.3460639 | 8.40852444 | 0 | 11.6532099 | 8.08118305 | 9.0180945 |
| Dtx1 | 0 | 0 | 0 | 0 | 0 | 0 |
| Dtx4 | 12.3828586 | 12.8400604 | 9.87791515 | 12.95339 | 0 | 0 |
| Ebf1 | 0 | 0 | 0 | 0 | 0 | 0 |
| Ep300 | 9.94498424 | 10.2010752 | 9.23583811 | 10.6282941 | 10.4403515 | 8.59444295 |
| Epor | 5.16793546 | 5.09166176 | 6.07340251 | 5.10546348 | 0 | 2.7151266 |
| Erg | 11.0543498 | 8.41211355 | 0 | 12.089156 | 10.1146713 | 11.7537883 |
| Esr1 | 11.6199962 | 10.7508391 | 0 | 10.3804934 | 10.0633516 | 0 |
| ETS1 | 0 | 11.8060427 | 0 | 2.87560829 | 10.507867 | 0 |
| ETS2 | 0 | 8.07791161 | 2.28329408 | 0.76338635 | 0 | 8.47008891 |
| Etv3 | 5.74740043 | 7.36604372 | 0 | 5.34860303 | 4.23394023 | 5.05619729 |
| Etv6 | 12.9684077 | 11.0021541 | 9.73755797 | 13.9096409 | 3.98851235 | 10.7091763 |
| Ezh2 | 11.2994093 | 9.96948763 | 8.77091516 | 11.243305 | 0 | 9.25661058 |
| Fas | 0 | 0 | 0 | 0 | 0 | 0 |
| Fcgr2b | 9.44038194 | 9.26444191 | 8.49671511 | 0 | 0 | 7.5507537 |
| Fcgr3 | 8.9878976 | 11.2705376 | 0 | 7.10105394 | 0 | 2.57719687 |
| Fli1 | 12.3237708 | 12.3248589 | 9.73909286 | 12.1105145 | 10.3593911 | 9.96450923 |
| Flt3 | 12.2416095 | 0 | 12.2385762 | 12.4225757 | 0 | 7.96248373 |
| Fosl1 | 0 | 0 | 0 | 8.07129215 | 0 | 0 |
| Foxo1 | 11.0340434 | 9.06969139 | 10.1546488 | 12.3061817 | 9.40775249 | 10.5472402 |
| Foxo3 | 9.90987077 | 7.70047424 | 0 | 11.2129013 | 10.4052826 | 9.57989143 |
| Gapdh | 14.3410656 | 13.3216214 | 6.17605235 | 13.0958987 | 9.71964182 | 8.2639086 |
| Gata1 | 0 | 1.6059749 | 0 | 0 | 0 | 0 |
| Gata2 | 4.56581362 | 0 | 0 | 3.24897579 | 5.55356347 | 6.52542185 |
| Gata3 | 8.22656643 | 0 | 0 | 0 | 8.13700583 | 7.25082557 |
| Gfi1 | 9.06056316 | 11.6538294 | 0 | 9.16221659 | 4.02040206 | 0 |
| Gfi1b | 0 | 0 | 0 | 0 | 0.25126544 | 0 |
| Hes5 | 0 | 0 | 0 | 0 | 5.25748063 | 0 |
| Hey1 | 0 | 0 | 0 | 0 | 0 | 0 |
| Hlf | 8.11935658 | 7.41139148 | 0 | 0 | 11.998899 | 13.3665089 |
| Id2 | 0 | 9.73284535 | 11.8927611 | 0 | 0 | 0 |
| Ifi203 | 10.9125767 | 1.56076385 | 10.965723 | 10.6623233 | 11.8059937 | 12.2738519 |
| Ifi205 | 0 | 7.49013979 | 11.8971931 | 0 | 0 | 0 |
| Ifitm1 | 9.61917406 | 0 | 0 | 11.9413193 | 13.1252834 | 12.4718304 |
| Ikzf1 | 11.9046539 | 11.2671835 | 10.14486 | 12.5650158 | 8.82268993 | 9.28321375 |
| Ikzf2 | 8.46869314 | 0 | 0 | 8.47986869 | 8.78289078 | 10.6878177 |
| Il7R | 0 | 0 | 0 | 0 | 0 | 0 |
| Irf4 | 0 | 0 | 0 | 0 | 0 | 0 |
| Irf6 | 4.12909642 | 2.27592361 | 0 | 0 | 5.77682646 | 0 |
| Irf8 | 12.9456699 | 0 | 13.624962 | 13.7405608 | 0 | 0 |
| Kdr | 0 | 0 | 0 | 0 | 0 | 0 |
| Kit | 14.0868279 | 13.592776 | 11.2221993 | 14.0164883 | 12.0391773 | 12.8744425 |
| Klf1 | 0 | 0 | 0 | 0 | 0 | 0 |
| Klf12 | 2.66660298 | 0 | 0 | 6.20104085 | 0 | 7.38482289 |
| Ldb1 | 12.285869 | 11.2819471 | 10.3063821 | 12.5631382 | 10.866794 | 10.6556867 |
| Lin28a | 5.33704681 | 6.88472231 | 6.51112264 | 2.23418642 | 0.05736793 | 4.0435655 |
| Lmo2 | 10.4881207 | 9.5635547 | 7.59010325 | 11.8118436 | 10.8803219 | 11.4475084 |
| Ly6a | 0 | 0 | 0 | 0 | 11.3195152 | 10.5870103 |
| Lyl1 | 8.61035099 | 7.73468542 | 0 | 10.6447346 | 0 | 7.65003858 |
| Mbd2 | 11.2505579 | 10.5919527 | 10.3145503 | 11.3020134 | 0 | 10.1428358 |
| Meis1 | 4.34410862 | 0 | 0 | 7.55516029 | 8.67866413 | 9.02711955 |
| Mllt3 | 0 | 0 | 0 | 0 | 7.12963107 | 2.87940553 |
| Mpl | 8.28694489 | 0 | 0 | 0 | 10.2778907 | 10.3627362 |
| Muc13 | 8.74153092 | 9.29662392 | 0 | 11.121408 | 5.14911074 | 8.76580934 |
| Myb | 13.9147396 | 14.2812014 | 0 | 14.2295701 | 10.5024756 | 11.003359 |
| Myc | 13.6235281 | 13.0901273 | 9.58950863 | 15.1619084 | 10.3020722 | 9.29939524 |
| Mycn | 0 | 7.0328665 | 0 | 0 | 7.93226454 | 8.80500295 |
| Ndn | 0 | 0 | 0 | 0 | 9.24126109 | 0 |
| Nfat5 | 9.56450436 | 10.5541109 | 5.24115849 | 7.8400374 | 7.82456966 | 9.32565577 |
| Nfia | 10.4800163 | 0 | 0 | 8.6877674 | 8.3554248 | 9.44711328 |
| Nfkb1 | 4.11854617 | 4.55346432 | 4.3546122 | 6.00282408 | 5.32088492 | 4.27063216 |
| Notch1 | 11.0427965 | 7.69294924 | 7.4684003 | 9.6813143 | 0 | 9.14014597 |
| Pax4 | 0 | 0 | 0 | 0 | 0 | 0 |
| Pax5 | 0 | 0 | 0 | 0 | 0 | 0 |
| Pax9 | 0 | 0 | 0 | 0 | 0 | 0 |
| Pbx1 | 0 | 2.41999393 | 0 | 4.60427348 | 0 | 0 |
| PIk3ca | 10.5133138 | 10.9351105 | 8.30194999 | 11.8682584 | 8.19933736 | 7.15189306 |
| PIk3R2 | 10.800657 | 9.31109965 | 8.14508176 | 9.89144953 | 0 | 8.29464733 |
| Plag1 | 0 | 0 | 0 | 0 | 8.62119125 | 8.41624245 |
| Prf1 | 6.25999009 | 0 | 0 | 0 | 0 | 0 |
| Pten | 9.85373481 | 10.1046387 | 8.4375715 | 11.8662431 | 8.3775621 | 9.78100476 |
| Rb1 | 11.4058642 | 10.524729 | 9.64537306 | 10.6398779 | 0 | 0 |
| Rora | 0 | 0 | 0 | 9.2194702 | 0 | 9.92254216 |
| Runx1 | 12.5823583 | 11.612649 | 0 | 13.1810639 | 8.13980404 | 0 |
| Runx2 | 6.07520491 | 4.62008078 | 3.85299235 | 8.15725883 | 5.48807374 | 4.3288158 |
| Satb1 | 10.3473077 | 10.4586335 | 0 | 12.8507889 | 0 | 0 |
| Sdpr | 0 | 0 | 0 | 0 | 0 | 0 |
| Sell | 13.1615763 | 11.0919349 | 8.27837081 | 12.9352801 | 0 | 0 |
| Sfpi1 | 12.2685432 | 12.3834981 | 11.8275651 | 12.5999867 | 9.7600535 | 0 |
| Slamf1 | 0 | 0 | 0 | 0 | 0 | 0 |
| Smarca4 | 13.9278719 | 12.4252093 | 11.4331679 | 14.0406109 | 10.7650413 | 10.4302513 |
| Sos1 | 7.08440665 | 9.19453302 | 0 | 8.82410076 | 0 | 7.80818117 |
| Stat1 | 7.33456058 | 8.62844753 | 3.26903654 | 4.37970726 | 2.44310501 | 2.24193334 |
| Stat3 | 11.6046184 | 12.0058285 | 10.4937808 | 10.7199143 | 10.1332837 | 11.4837559 |
| Stat4 | 9.89970671 | 8.7484529 | 0 | 10.0534291 | 7.14597799 | 8.52079622 |
| Stat6 | 10.0340055 | 7.76884318 | 9.26899604 | 8.52011684 | 0 | 0 |
| Suz12 | 12.1917303 | 10.6415578 | 0 | 11.4192066 | 0 | 10.1796014 |
| Tal1 | 3.27202494 | 2.33635462 | 5.43421365 | 1.99510515 | 3.23551253 | 7.24054415 |
| Tcf3 | 8.95886195 | 9.27584441 | 7.18949224 | 7.95247356 | 0 | 5.94183007 |
| Tcf4 | 11.7535018 | 10.7218079 | 5.87396176 | 13.0570735 | 10.2194603 | 10.2598245 |
| Tcf7 | 0 | 0 | 0 | 0 | 0 | 0 |
| Tek | 0 | 0 | 0 | 0 | 8.12191874 | 0 |
| Tfrc | 11.1276806 | 11.6773601 | 0 | 9.99135979 | 0 | 2.48510433 |
| Tgfb1 | 8.59999451 | 7.86082222 | 0 | 7.41061996 | 0 | 0 |
| Tgfb2 | 0 | 0 | 0 | 5.61040412 | 0 | 0 |
| Tgfb3 | 0 | 1.64625868 | 0 | 0 | 8.66536386 | 0 |
| Tnfrsf1a | 12.1075835 | 11.7893286 | 10.2883436 | 12.9872996 | 9.70789834 | 10.0685048 |
| Tnfrsf1b | 11.1644655 | 10.6687255 | 0 | 10.8829595 | 0 | 8.01385336 |
| Tnfrsf21 | 7.9588553 | 8.25912716 | 0 | 6.93837391 | 5.31291687 | 0 |
| Tnfsf10 | 7.2217542 | 0 | 0 | 6.57504105 | 0 | 6.42935948 |
| Tnfsf12 | 6.10886882 | 0 | 0 | 5.7030187 | 0 | 0 |
| Tob1 | 4.89785115 | 0 | 4.30862997 | 1.32359285 | 1.07788382 | 0 |
| vWF | 0 | 0 | 0 | 0 | 0 | 6.1655458 |
| Zbtb20 | 7.51328071 | 0 | 8.49995327 | 7.62054695 | 8.85871267 | 9.72768241 |
| Zbtb38 | 9.44025595 | 10.3426011 | 7.11037442 | 10.7447144 | 8.87190914 | 8.84029249 |
| Zfp532 | 0 | 0 | 0 | 0 | 0 | 0 |
| Zfp612 | 0 | 5.18701551 | 7.05359804 | 3.11635926 | 0 | 5.7890343 |
| Zfpm1 | 0 | 0 | 0 | 0 | 0 | 0 |
| Zhx2 | 0 | 5.27170259 | 0 | 0 | 0 | 0 |

TABLE 6-8
Single cell expression data (reduced list)—Control

| Factor | HSC3 | HSC4 | HSC5 | HSC6 | HSC7 | HSC8 |
|---|---|---|---|---|---|---|
| Actb | 13.577974 | 14.0296483 | 14.1103469 | 15.5819895 | 15.4017467 | 14.5186085 |
| Aebp2 | 6.10559528 | 5.88912085 | 4.6132596 | 6.72522268 | 6.54183737 | 6.53821191 |
| Ahr | 0 | 8.48413666 | 0 | 8.64794663 | 0 | 0 |
| Akt1 | 5.7101674 | 8.39335711 | 8.11021366 | 10.2087847 | 8.77360611 | 9.23696389 |
| Akt2 | 0 | 0 | 0 | 5.73394549 | 4.95527812 | 5.5482851 |
| Akt3 | 8.79551486 | 1.55468933 | 8.24574153 | 9.13533117 | 9.22444783 | 8.23443739 |
| APC | 0 | 0 | 0 | 9.1544444 | 8.26372086 | 0 |
| Bad | 0 | 0 | 0 | 0 | 0 | 0 |
| Bax | 10.4587872 | 7.84637341 | 8.21704944 | 10.5910972 | 9.05419378 | 8.1433208 |
| Bcl11a | 0 | 0 | 0 | 0 | 0 | 8.71685996 |
| Bcl11b | 0 | 0 | 0 | 0 | 0 | 0 |
| Bcl2 | 0 | 0 | 0 | 6.60286713 | 0 | 0 |
| Bcl2l1 | 0 | 8.15463837 | 0 | 8.81750986 | 9.51798174 | 9.26348136 |
| Bcl2l11 | 0 | 7.08014318 | 0 | 0 | 8.80771493 | 0 |
| Bmi1 | 6.37303271 | 6.75760763 | 6.40723471 | 8.78539598 | 6.73467101 | 0 |
| Brd3 | 8.10648223 | 9.12195615 | 0 | 10.313197 | 9.04032119 | 8.4172914 |
| Casp8 | 8.60911844 | 8.67718647 | 8.08973581 | 8.8351678 | 8.29348209 | 10.4887846 |
| Casp9 | 8.50198655 | 0 | 0 | 8.0906086 | 8.93408591 | 0 |
| Cbx2 | 2.12580066 | 0 | 1.37858473 | 0 | 6.38626502 | 3.95391221 |
| Cbx8 | 0 | 0 | 0 | 0 | 0 | 0 |
| Ccnc | 8.0612119 | 7.75585225 | 0 | 8.0425277 | 7.97210372 | 4.50082307 |
| Ccnd1 | 0 | 9.44185728 | 0 | 10.806783 | 0 | 9.84865359 |
| Ccne2 | 0 | 0 | 0 | 0 | 0 | 0 |
| CD34 | 8.17751775 | 5.00363076 | 7.74656357 | 7.72536834 | 7.31850948 | 0 |
| CD41 | 0 | 0 | 0 | 10.2838042 | 0 | 10.3942665 |
| CD48 | 0 | 0 | 0 | 0 | 0 | 0 |
| CD52 | 0 | 8.30090194 | 0 | 0 | 0 | 0 |
| CD53 | 0 | 0 | 0 | 0 | 0 | 0 |
| CD55 | 7.69179367 | 4.79347239 | 6.9936477 | 9.05205329 | 0 | 8.21658095 |
| CD63 | 8.84869188 | 9.80818054 | 8.85251987 | 10.377284 | 8.91902336 | 8.99037439 |
| CD9 | 7.96692234 | 7.15928214 | 7.1345801 | 8.5320473 | 3.5188154 | 8.2765401 |
| Cdc42 | 11.8342425 | 11.274525 | 11.5477464 | 12.9667945 | 11.216272 | 12.9992851 |
| Cdk1 | 0 | 0 | 1.70469042 | 9.19399937 | 0 | 8.58515514 |
| Cdk4 | 6.80715808 | 7.17264944 | 2.02643408 | 11.1452163 | 9.41268282 | 6.45109978 |
| Cdkn2b | 0 | 0 | 0 | 0 | 0 | 0 |
| Cebpa | 8.66392034 | 0 | 8.58072977 | 6.63194812 | 0 | 0 |
| Csf1r | 0 | 0 | 8.74066681 | 1.70542256 | 7.47370204 | 0 |
| Ctnnb1 | 6.45093961 | 6.80576451 | 7.03105301 | 8.66585445 | 4.63621377 | 6.42492055 |
| Cycs | 7.76931122 | 8.17385953 | 9.1062029 | 11.5938916 | 10.2963567 | 10.5610571 |
| Dach1 | 8.32689948 | 9.6993744 | 0 | 10.5160163 | 11.5555411 | 12.1784951 |
| Dnmt1 | 0 | 0 | 0 | 11.5088913 | 0 | 10.870094 |
| Dnmt3a | 10.0217648 | 11.1560578 | 9.24043447 | 10.2575566 | 10.2648603 | 12.1222467 |
| Dnmt3b | 0 | 0 | 0 | 8.90491552 | 0 | 9.12251996 |
| Dtx1 | 3.63908589 | 0.2314944 | 3.28281301 | 0 | 0 | 1.84006193 |
| Dtx4 | 0 | 0 | 0 | 0 | 1.19632544 | 0 |
| Ebfl | 0 | 0 | 0 | 0 | 0 | 0 |
| Ep300 | 11.0845039 | 8.98243523 | 10.7104073 | 9.62872537 | 9.96024059 | 9.41340549 |
| Epor | 4.04169265 | 5.05457514 | 6.15980606 | 4.89038806 | 5.63286624 | 5.89050554 |
| Erg | 11.8077154 | 11.2396194 | 11.3083977 | 11.0154674 | 10.8697562 | 10.0863194 |
| Esr1 | 8.38535842 | 0 | 9.45876416 | 0 | 8.20146951 | 9.59278249 |
| ETS1 | 7.78767496 | 8.3813926 | 8.32316912 | 0 | 0 | 0 |
| ETS2 | 0 | 5.54640271 | 0 | 9.236687 | 0 | 10.2058893 |
| Etv3 | 1.54998505 | 6.21266641 | 4.23572008 | 6.55515366 | 0 | 3.67608709 |
| Etv6 | 10.2492298 | 11.658684 | 11.1884801 | 12.4484167 | 10.2573908 | 11.513336 |
| Ezh2 | 0 | 6.45902485 | 8.45850492 | 9.86622345 | 6.62197678 | 0 |
| Fas | 0 | 0 | 0 | 0 | 0 | 0 |
| Fcgr2b | 0 | 0 | 0 | 3.00096067 | 0 | 0 |
| Fcgr3 | 0 | 0 | 0 | 0 | 0 | 0 |
| Fli1 | 10.825293 | 10.3056342 | 10.1656639 | 12.7030871 | 9.81370266 | 10.7815026 |
| Flt3 | 0 | 0 | 0 | 0 | 8.83959351 | 0 |
| Fosl1 | 0 | 5.63779061 | 0 | 9.84241504 | 0 | 0 |
| Foxo1 | 11.1098742 | 10.8687068 | 10.3544835 | 11.2304826 | 9.6589649 | 11.609313 |
| Foxo3 | 8.96881644 | 9.34207286 | 0 | 10.574468 | 7.95875599 | 10.5612825 |
| Gapdh | 10.3938142 | 10.020788 | 9.78199569 | 11.7324163 | 11.2583198 | 10.2840324 |
| Gata1 | 3.66598041 | 1.2604332 | 0 | 0 | 0 | 8.0389608 |
| Gata2 | 4.10700961 | 5.22811433 | 6.14699434 | 5.75841883 | 6.0549266 | 5.76445634 |
| Gata3 | 6.39172576 | 0 | 8.61417098 | 7.96956347 | 7.63953107 | 8.62787032 |
| Gfi1 | 0 | 0 | 0 | 0 | 0 | 0 |
| Gfi1b | 10.6982479 | 8.35858247 | 0 | 9.76814181 | 0 | 9.06455865 |
| Hes5 | 0 | 0 | 0 | 0 | 0 | 0 |
| Hey1 | 2.14957956 | 0 | 0 | 5.41172737 | 3.30247516 | 0 |
| Hlf | 12.2869167 | 12.3244122 | 12.7023562 | 11.4515454 | 12.4604982 | 12.6666107 |
| Id2 | 7.39149179 | 0 | 0 | 0 | 0 | 7.98972755 |
| Ifi203 | 12.5769615 | 12.1345502 | 12.0725801 | 11.3590361 | 12.2927044 | 11.1325428 |
| Ifi205 | 0 | 0 | 0 | 0 | 0 | 0 |
| Ifitm1 | 13.1901123 | 12.7092713 | 11.5835195 | 13.4449774 | 11.4136686 | 13.2554104 |
| Ikzf1 | 9.7363741 | 9.85625177 | 0 | 10.250229 | 9.90890256 | 8.72152915 |
| Ikzf2 | 10.2835862 | 9.22641485 | 0 | 8.77854263 | 0 | 7.21339614 |
| Il7R | 0 | 0 | 0 | 0 | 0 | 0 |
| Irf4 | 0 | 0 | 0 | 0 | 0 | 0 |
| Irf6 | 3.19293607 | 0 | 3.18045922 | 3.89301413 | 3.20245453 | 4.43165432 |
| Irf8 | 0 | 7.94066203 | 0 | 0 | 7.94125807 | 2.2099363 |
| Kdr | 0 | 0 | 0 | 0 | 0 | 10.4341118 |
| Kit | 11.6075917 | 11.3831769 | 13.0210319 | 12.1707632 | 12.6193513 | 11.9124539 |
| Klf1 | 0 | 0 | 0 | 0 | 0 | 0 |
| K1f12 | 0 | 8.1606124 | 0 | 8.75728664 | 2.77952504 | 8.68882167 |
| Ldb1 | 10.7018656 | 10.5501009 | 10.3395739 | 11.8108028 | 11.1819378 | 11.9298611 |
| Lin28a | 0 | 0.65022055 | 8.32375974 | 0 | 0 | 8.92276223 |
| Lmo2 | 11.1260842 | 11.2055555 | 11.2304278 | 11.1718854 | 10.0919978 | 9.95604882 |
| Ly6a | 11.6807743 | 13.0059956 | 0 | 11.8972718 | 9.8150555 | 8.15846371 |
| Lyl1 | 7.71114163 | 0 | 9.39299973 | 5.87215945 | 0 | 7.14723677 |
| Mbd2 | 4.92840001 | 0 | 9.40592756 | 8.95643535 | 8.35525208 | 10.4778257 |
| Meis1 | 9.33744894 | 7.24719639 | 8.11655673 | 9.24808657 | 9.02869584 | 8.20607406 |
| Mllt3 | 4.39596095 | 0 | 0 | 4.84582581 | 2.28189221 | 1.22539492 |
| Mpl | 8.37937771 | 11.6344232 | 10.240321 | 9.1484092 | 9.92137235 | 11.7759292 |
| Muc13 | 5.85943592 | 8.69171484 | 5.79478348 | 8.86606586 | 8.08439421 | 5.87402461 |
| Myb | 11.4543645 | 11.6634674 | 11.813638 | 12.4301573 | 11.6054666 | 10.9746986 |
| Myc | 0 | 9.07187777 | 10.4973302 | 12.1156989 | 11.1821332 | 0 |
| Mycn | 10.0040447 | 1.76067461 | 8.7209187 | 11.9081484 | 9.172818 | 13.4121675 |
| Ndn | 11.413717 | 0 | 0 | 11.1011159 | 8.47770715 | 0 |
| Nfat5 | 8.80140323 | 8.27575413 | 8.94488444 | 10.4915077 | 7.87669831 | 8.9488905 |
| Nfia | 9.26039859 | 8.37576634 | 8.54427003 | 9.80432597 | 10.4688522 | 9.95162743 |
| Nfkb1 | 4.28180114 | 1.0386031 | 0 | 4.30632205 | 4.27397363 | 0 |
| Notch1 | 0 | 0 | 7.85740045 | 0 | 0 | 0 |
| Pax4 | 0 | 0 | 0 | 0 | 0 | 0 |
| Pax5 | 0 | 0 | 0 | 0.34067989 | 0 | 0 |
| Pax9 | 0 | 0 | 0 | 0 | 0 | 0 |
| Pbx1 | 0 | 0 | 0 | 0 | 0 | 0 |
| PIk3ca | 9.48126253 | 8.2821557 | 10.3094662 | 8.07275737 | 0 | 8.54082063 |
| PIk3R2 | 0 | 0 | 9.46846214 | 8.95184962 | 0 | 0 |
| Plag1 | 8.44717703 | 0 | 8.62974666 | 6.40451656 | 10.2884491 | 9.70437763 |
| Prf1 | 0 | 0 | 0 | 0 | 3.42401778 | 0 |
| Pten | 9.35030834 | 5.62716649 | 8.59897884 | 8.41844617 | 9.21702967 | 8.85833533 |
| Rb1 | 9.73808815 | 0 | 9.45856621 | 10.3613325 | 5.17427811 | 4.88975979 |
| Rora | 0 | 8.26236355 | 10.2950769 | 9.73645132 | 0 | 0 |
| Runx1 | 9.69584379 | 8.40584267 | 10.6007548 | 0 | 10.9238866 | 8.69978638 |
| Runx2 | 4.75896314 | 5.38267048 | 0 | 6.2671313 | 7.04999695 | 0 |
| Satb1 | 9.80742018 | 0 | 0 | 8.57255153 | 0 | 0 |
| Sdpr | 0 | 0 | 0 | 0 | 0 | 3.04221011 |
| Sell | 0 | 0 | 0 | 0 | 0 | 0 |
| Sfpi1 | 10.1339723 | 10.3780048 | 10.4866679 | 0 | 10.0355372 | 9.31766744 |
| Slamf1 | 7.9591016 | 0 | 0 | 0 | 0 | 9.42150329 |
| Smarca4 | 9.57780054 | 9.85977591 | 10.4104054 | 11.7238705 | 9.46298092 | 11.1339334 |
| Sos1 | 0 | 8.2843901 | 0 | 7.22160003 | 7.53838311 | 7.29089291 |
| Sta1 | 2.50659523 | 6.02858174 | 2.36927337 | 3.71643375 | 2.2740799 | 12.52689091 |
| Stat3 | 8.29378181 | 10.7345608 | 9.55246631 | 10.8963074 | 10.3681668 | 8.97518786 |
| Stat4 | 8.4227657 | 7.83127215 | 8.8192144 | 10.1874616 | 9.68055604 | 7.69477544 |
| Stat6 | 0 | 10.5487746 | 9.40667371 | 9.72923693 | 9.87383314 | 9.84674959 |
| Suz12 | 9.05894393 | 0 | 8.44467624 | 10.0115616 | 8.21144523 | 9.0031541 |
| Tal1 | 0 | 3.75390852 | 0 | 7.11083842 | 5.49903472 | 3.85114596 |
| Tcf3 | 0 | 0 | 0 | 0 | 3.17986266 | 4.49147102 |
| Tcf4 | 10.6387202 | 9.81058079 | 10.1324014 | 10.7265873 | 9.19540096 | 11.280981 |
| Tcf7 | 0 | 0 | 4.14707011 | 0 | 0 | 0 |
| Tek | 8.14772158 | 0 | 7.22964189 | 6.69314683 | 7.21296798 | 0 |
| Tfrc | 5.69240635 | 8.85266347 | 0 | 8.37463351 | 0 | 0 |
| Tgfb1 | 0 | 0 | 0 | 0 | 7.01325075 | 0 |
| Tgfb2 | 0 | 0 | 0 | 0 | 0 | 0 |
| Tgfb3 | 0 | 0 | 0 | 0 | 5.62887852 | 0 |
| Tnfrsf1a | 9.20893254 | 7.99674932 | 8.49210484 | 9.09094768 | 8.72598965 | 8.95788173 |
| Tnfrsf1b | 10.4438284 | 0 | 8.3857973 | 3.73738697 | 8.30125396 | 0 |
| Tnfrsf2l | 4.92370115 | 6.91456141 | 5.18164833 | 6.87061679 | 0 | 5.73392501 |
| Tnfsf10 | 0 | 0 | 7.01737198 | 4.03273131 | 0 | 5.00807925 |
| Tnfsf12 | 0 | 0 | 0 | 0 | 0 | 0 |
| Tob1 | 5.71122498 | 7.95080155 | 0 | 6.10121099 | 7.42164007 | 0 |
| vWF | 3.86680517 | 0 | 0 | 0 | 4.46829427 | 8.91506154 |
| Zbtb20 | 8.16491305 | 7.28441527 | 7.64424277 | 3.60015575 | 8.29513002 | 8.35006537 |
| Zbtb38 | 0 | 7.67871493 | 9.19649825 | 8.51227823 | 9.98861231 | 7.66701854 |
| Zfp532 | 0 | 4.08592807 | 3.77146991 | 4.36860224 | 0 | 2.64992417 |
| Zfp612 | 5.04540623 | 1.29781735 | 6.43562895 | 1.81941986 | 0 | 5.71057878 |
| Zfpm1 | 0 | 0 | 0 | 0 | 7.43302501 | 0 |
| Zhx2 | 0 | 3.17433055 | 0 | 0 | 0 | 10.2241584 |

TABLE 6-9
Single cell expression data (reduced list)—Control

| Factor | HSC9 | HSC10 | MEP1 | MEP2 | MEP3 | MEP4 |
|---|---|---|---|---|---|---|
| Actb | 14.9725561 | 15.5430056 | 16.6739018 | 17.1798405 | 16.7754755 | 16.9120965 |
| Aebp2 | 5.34272666 | 2.46759537 | 7.60291615 | 5.68775766 | 8.40647947 | 8.15032471 |
| Ahr | 0 | 0 | 0 | 0 | 0 | 0 |
| Akt1 | 8.71552396 | 9.04361278 | 10.8964237 | 10.6593665 | 10.5554637 | 10.4625715 |
| Akt2 | 1.6860339 | 0 | 5.40370098 | 7.79517803 | 7.18806974 | 6.57237902 |
| Akt3 | 9.27378957 | 9.16410517 | 0 | 7.90778801 | 7.25351311 | 8.6899408 |
| APC | 0 | 0 | 0 | 0 | 0 | 8.92917564 |
| Bad | 0 | 0 | 0 | 0 | 0 | 0 |
| Bax | 9.57334173 | 9.01870701 | 13.1654553 | 12.6147597 | 11.6763189 | 11.8499785 |
| Bcl11a | 0 | 6.38030957 | 8.61622865 | 0 | 0 | 6.50601386 |
| Bcl11b | 0 | 0 | 0 | 3.99048372 | 0 | 0 |
| Bcl2 | 6.53694296 | 5.97214969 | 0 | 0 | 0 | 0 |
| Bcl2l1 | 10.5706275 | 2.81256542 | 0 | 9.90189687 | 7.93964747 | 0 |
| Bcl2l11 | 0 | 0 | 11.4930521 | 9.68858479 | 11.1719166 | 11.744598 |
| Bmi1 | 8.0356025 | 5.71483882 | 8.52931514 | 8.55595556 | 10.0673986 | 8.43810889 |
| Brd3 | 11.4628865 | 8.46832128 | 11.0280089 | 11.2582907 | 10.1577315 | 11.3931352 |
| Casp8 | 9.80815784 | 10.7239994 | 8.89508957 | 6.21772996 | 7.84127145 | 10.4709266 |
| Casp9 | 0 | 0 | 5.82312549 | 10.5005325 | 10.3674251 | 10.8167842 |
| Cbx2 | 2.20378454 | 5.19558249 | 5.96803494 | 4.9871259 | 0 | 1.36472366 |
| Cbx8 | 0 | 0 | 0 | 0 | 0 | 0 |
| Ccnc | 7.14555417 | 9.54460991 | 9.10840098 | 7.7775943 | 9.69822219 | 10.7463612 |
| Ccnd1 | 10.3147623 | 8.43483043 | 12.5806504 | 10.1616065 | 0 | 9.50499479 |
| Ccne2 | 2.11634283 | 0 | 12.3632828 | 11.5458361 | 7.65370744 | 11.8196672 |
| CD34 | 8.7300738 | 7.56097552 | 0 | 0 | 0 | 0 |
| CD41 | 9.90770066 | 10.2820486 | 0 | 0 | 0 | 0 |
| CD48 | 0 | 0 | 8.8519551 | 0 | 0 | 10.2839816 |
| CD52 | 0 | 0 | 0 | 0 | 0 | 0 |
| CD53 | 0 | 0 | 0 | 0 | 0 | 0 |
| CD55 | 0 | 0 | 9.35100587 | 9.53334636 | 6.17916642 | 8.52837797 |
| CD63 | 9.95525539 | 9.13287496 | 0 | 0 | 0 | 0 |
| CD9 | 8.19006868 | 8.93484354 | 0 | 0 | 0 | 0 |
| Cdc42 | 12.8484097 | 12.3557558 | 14.0139592 | 14.5300457 | 13.7188884 | 14.3827631 |
| Cdk1 | 8.40315409 | 0 | 10.2066902 | 12.0503625 | 11.856245 | 10.4737341 |
| Cdk4 | 9.36174345 | 9.55697505 | 12.4311347 | 12.9555662 | 12.0600059 | 13.2462207 |
| Cdkn2b | 0 | 0 | 0 | 7.13192772 | 0 | 0 |
| Cebpa | 0 | 0 | 4.83137386 | 0 | 6.06861889 | 0 |
| Csf1r | 0.78905294 | 5.76347829 | 7.35935898 | 0 | 0 | 0.43693507 |
| Ctnnb1 | 6.91786038 | 7.30835446 | 9.09223561 | 9.17717471 | 8.27674053 | 9.66975154 |
| Cycs | 9.20845458 | 9.97537598 | 14.4833117 | 14.8868575 | 14.3632329 | 14.6822205 |
| Dach1 | 11.8391027 | 11.4620792 | 0 | 8.08586478 | 0 | 8.85421269 |
| Dnmt1 | 0 | 0 | 13.0436858 | 12.9585708 | 12.173667 | 12.6846992 |
| Dnmt3a | 10.6627832 | 10.9853226 | 0 | 0 | 8.14594383 | 11.3793103 |
| Dnmt3b | 8.4439035 | 7.87008545 | 9.13349446 | 10.0430891 | 0 | 8.58078692 |
| Dtx1 | 0 | 0 | 0 | 0 | 0 | 0 |
| Dtx4 | 0 | 8.77381733 | 0 | 3.59988975 | 0 | 0 |
| Ebf1 | 0 | 0 | 0 | 0 | 0 | 0 |
| Ep300 | 10.2826339 | 9.35773902 | 9.97912263 | 9.64315597 | 7.04223723 | 9.25408444 |
| Epor | 5.62014836 | 3.32438848 | 6.0772899 | 6.71011031 | 7.0106626 | 6.12830238 |
| Erg | 11.4258262 | 11.2053622 | 0 | 0 | 0 | 0 |
| Esr1 | 9.13193309 | 9.35521874 | 0 | 0 | 0 | 0 |
| ETS1 | 8.08986929 | 4.00036102 | 0 | 0 | 0 | 3.28475493 |
| ETS2 | 8.93566794 | 8.12463187 | 8.26008629 | 7.11961974 | 8.01954074 | 9.00778633 |
| Etv3 | 4.73179257 | 6.37769317 | 0 | 0 | 0 | 0 |
| Etv6 | 10.868184 | 11.7795506 | 0 | 0 | 0 | 9.64836021 |
| Ezh2 | 0 | 2.07633721 | 11.8577244 | 11.7699702 | 9.0854248 | 11.235091 |
| Fas | 0 | 0 | 0 | 0 | 0 | 0 |
| Fcgr2b | 0 | 0 | 0 | 0 | 0 | 0 |
| Fcgr3 | 0 | 0 | 0 | 0 | 0 | 0 |
| Fli1 | 12.2531926 | 10.9251156 | 0 | 0 | 0 | 1.16488259 |
| Flt3 | 0 | 0 | 0 | 0 | 0 | 0 |
| Fosl1 | 0 | 0 | 0.29931482 | 9.04736396 | 0 | 0 |
| Foxo1 | 9.01385813 | 10.4459955 | 5.73742257 | 0 | 0 | 9.36919814 |
| Foxo3 | 8.79125674 | 8.68822067 | 9.68364766 | 10.649167 | 11.0869714 | 10.3601155 |
| Gapdh | 11.7285693 | 11.1757682 | 13.63523 | 13.4830598 | 13.1943802 | 11.9720384 |
| Gata1 | 5.94283713 | 0 | 12.3245112 | 13.7649949 | 12.797531 | 13.2227802 |
| Gata2 | 6.83304794 | 6.87340412 | 2.25249885 | 0 | 0 | 3.77741299 |
| Gata3 | 9.24060916 | 9.14520142 | 0 | 0 | 0 | 0 |
| Gfi1 | 3.34268315 | 0 | 0 | 0 | 0 | 4.24919213 |
| Gfi1b | 9.56101095 | 9.49767669 | 13.5283046 | 14.1818634 | 13.7733661 | 14.9174041 |
| Hes5 | 0 | 0 | 0 | 2.29471695 | 0 | 0 |
| Hey1 | 0 | 0 | 0 | 1.95932422 | 0 | 0 |
| Hlf | 12.1074683 | 11.9161928 | 0 | 0 | 0 | 0 |
| Id2 | 0 | 0 | 0 | 0 | 0 | 0 |
| Ifi203 | 12.278004 | 11.192533 | 0 | 0 | 0 | 0 |
| Ifi205 | 0 | 0 | 0 | 0 | 0 | 0 |
| Ifitm1 | 13.0046432 | 12.4025715 | 0.31225789 | 0 | 0 | 0 |
| Ikzf1 | 9.53642019 | 0 | 12.6360198 | 12.9519677 | 12.6541472 | 12.6965722 |
| Ikzf2 | 0 | 8.56779353 | 5.40027468 | 0 | 0 | 0 |
| Il7R | 0 | 0 | 0 | 0 | 0 | 0 |
| Irf4 | 0 | 0 | 0 | 0 | 0 | 0 |
| Irf6 | 4.10562562 | 0 | 0 | 0 | 0 | 0 |
| Irf8 | 0 | 0 | 6.12370867 | 8.41676829 | 0 | 0 |
| Kdr | 0 | 0 | 0 | 0 | 0 | 0 |
| Kit | 12.3160333 | 9.89526777 | 12.0150326 | 9.98117872 | 11.4951247 | 12.8352736 |
| Klf1 | 0 | 0 | 13.1692563 | 13.8873887 | 13.1692722 | 13.1308469 |
| Klf12 | 9.72448024 | 0 | 0 | 0 | 0 | 7.34581981 |
| Ldb1 | 12.4178652 | 11.01075 | 13.0390188 | 13.7874605 | 13.1564759 | 13.2168482 |
| Lin28a | 3.82329997 | 6.81496961 | 7.33729444 | 6.61281699 | 0 | 6.08888311 |
| Lmo2 | 11.5637423 | 9.92019304 | 11.5030529 | 11.9384772 | 12.6434079 | 10.6947974 |
| Ly6a | 7.06884075 | 8.44508253 | 0 | 0 | 0 | 0 |
| Lyl1 | 8.88014622 | 8.35784347 | 6.95117372 | 9.77435583 | 0 | 9.20859202 |
| Mbd2 | 9.13439987 | 8.56907105 | 13.0509809 | 13.9543519 | 12.6665248 | 13.1243311 |
| Meis1 | 9.80331618 | 8.1571184 | 0 | 0 | 0 | 0 |
| Mllt3 | 4.15938546 | 0 | 4.12160509 | 7.79510244 | 0 | 4.48253793 |
| Mpl | 10.996255 | 9.44406546 | 0 | 0 | 0 | 0 |
| Muc13 | 6.82422246 | 7.13195827 | 0 | 0 | 5.01336678 | 1.81337571 |
| Myb | 11.5244445 | 12.4461891 | 13.2111197 | 12.8071283 | 12.9764179 | 13.9759288 |
| Myc | 0 | 10.9160751 | 11.9866355 | 11.2429304 | 12.3999539 | 13.9029694 |
| Mycn | 14.4756491 | 11.6026038 | 0 | 0 | 0 | 0 |
| Ndn | 11.2837686 | 10.4369415 | 0 | 0 | 0 | 0 |
| Nfat5 | 9.61855366 | 6.82014528 | 6.59682409 | 7.27479713 | 0 | 8.74499807 |
| Nfia | 9.41329393 | 10.6171397 | 13.5009826 | 14.0902354 | 12.9714138 | 13.0254549 |
| Nfkb1 | 0 | 2.2519501 | 3.25804287 | 2.4908206 | 0 | 5.47964873 |
| Notch1 | 8.12991025 | 0 | 0 | 0 | 0 | 0 |
| Pax4 | 6.1516811 | 0 | 4.85665083 | 0 | 0 | 3.3619616 |
| Pax5 | 0 | 0 | 0 | 0 | 0 | 0 |
| Pax9 | 0 | 0 | 0 | 0 | 0 | 0 |
| Pbx1 | 0 | 0 | 0 | 0 | 0 | 0 |
| Plk3ca | 9.25738741 | 8.96174345 | 10.7256565 | 10.8683249 | 9.65150968 | 11.2464339 |
| PIk3R2 | 9.30544358 | 0 | 12.2128948 | 12.2784314 | 11.0187609 | 12.287832 |
| Plagl | 8.64324095 | 0 | 9.06569451 | 0 | 0 | 0 |
| Prf1 | 0 | 0 | 0 | 6.84326582 | 0 | 0 |
| Pten | 9.60478178 | 9.6731031 | 10.2929626 | 10.3569939 | 10.4823987 | 9.95159857 |
| Rb1 | 10.2970029 | 8.60735432 | 12.6978008 | 13.4211639 | 10.6504251 | 12.7561166 |
| Rora | 0 | 0 | 0 | 0 | 0 | 0 |
| Runx1 | 10.1584718 | 9.33038616 | 10.7805682 | 8.07026179 | 0 | 9.37272993 |
| Runx2 | 4.28423467 | 4.26402635 | 0 | 0 | 0 | 0 |
| Satb1 | 0 | 0 | 0 | 0 | 0 | 0 |
| Sdpr | 4.32744503 | 1.7057899 | 0 | 0 | 0 | 0 |
| Sell | 0 | 0 | 0 | 0 | 0 | 0 |
| Sfpi1 | 6.83682861 | 11.0545418 | 0 | 5.45218154 | 2.04252139 | 0 |
| Slamf1 | 9.49023226 | 0 | 0 | 0 | 0 | 0 |
| Smarca4 | 12.2973535 | 11.2507486 | 13.5426414 | 13.619892 | 12.1620756 | 12.8704926 |
| Sos1 | 8.06475892 | 6.27720781 | 9.19936975 | 7.36566754 | 6.8048466 | 9.35683189 |
| Stat1 | 4.52662558 | 0.99719233 | 2.64761229 | 4.45186216 | 3.7746722 | 7.81972053 |
| Stat3 | 11.0439439 | 11.5366037 | 8.88331309 | 0 | 9.64846927 | 9.55859823 |
| Stat4 | 9.34715798 | 6.79032745 | 6.31260327 | 0 | 0 | 0 |
| Stat6 | 11.0298664 | 7.72102603 | 11.0611639 | 8.69939135 | 9.35073565 | 2.30375272 |
| Suz12 | 9.21902863 | 9.76884858 | 13.4162816 | 13.3069763 | 12.1189393 | 12.8721225 |
| Tal1 | 3.5749488 | 2.63840682 | 5.48831658 | 5.90135703 | 5.14302435 | 6.08051416 |
| Tcf3 | 8.19970314 | 7.80579899 | 10.3443653 | 9.82695879 | 7.84599927 | 11.004031 |
| Tcf4 | 11.8040378 | 9.82961636 | 10.260851 | 11.3188496 | 11.2508544 | 11.653967 |
| Tcf7 | 0 | 4.73697982 | 1.43010499 | 0 | 0 | 0 |
| Tek | 9.36503436 | 0 | 0 | 0 | 0 | 0 |
| Tfrc | 0 | 8.8168966 | 13.9704118 | 14.578062 | 12.3681007 | 13.856442 |
| Tgfb1 | 0 | 5.44659661 | 0 | 6.89639764 | 0 | 7.19033201 |
| Tgfb2 | 0 | 0 | 5.36997264 | 0 | 0 | 0 |
| Tgfb3 | 8.5895152 | 11.83410769 | 0 | 0 | 0 | 0 |
| Tnfrsf1a | 11.0327854 | 10.3930716 | 0 | 0 | 0 | 9.18248017 |
| Tnfrsf1b | 8.64142351 | 8.04393607 | 0 | 0 | 0 | 0 |
| Tnfrsf21 | 6.97043206 | 6.09809349 | 0 | 5.3555269 | 0 | 4.45599329 |
| Tnfsf10 | 6.74936749 | 5.83080275 | 6.33722942 | 0 | 0 | 6.31492348 |
| Tnfsf12 | 0 | 0 | 0 | 0 | 0 | 0 |
| Tob1 | 5.23114212 | 0 | 7.58798007 | 0 | 8.41045357 | 8.34798458 |
| vWF | 8.26879091 | 5.5354592 | 3.98254277 | 0 | 0 | 0 |
| Zbtb20 | 6.7194969 | 9.77047876 | 0 | 6.66889471 | 0 | 0 |
| Zbtb38 | 9.66819933 | 8.29751972 | 10.3083101 | 9.74280335 | 8.68702379 | 10.8279681 |
| Zfp532 | 0 | 0 | 3.7690821 | 2.38462111 | 0 | 4.07815427 |
| Zfp612 | 6.99532728 | 0 | 0 | 0 | 0 | 6.43937583 |
| Zfpm1 | 6.81923051 | 6.31482951 | 8.84469315 | 0 | 7.30319601 | 10.1270265 |
| Zhx2 | 8.4994904 | 0 | 0 | 1.62381423 | 0 | 0 |

TABLE 6-10
Single cell expression data (reduced list)—Control

| Factor | MEP5 | MEP6 | MEP7 | MEP8 | MEP9 | MEP10 |
|---|---|---|---|---|---|---|
| Actb | 17.2576396 | 17.1978808 | 15.5072422 | 17.1016623 | 17.0883469 | 16.1373068 |
| Aebp2 | 8.8914175 | 8.24109539 | 5.927731 | 8.12926334 | 6.58436041 | 7.2192823 |
| Ahr | 0 | 0 | 0 | 0 | 0 | 7.10578696 |
| Akt1 | 11.6018488 | 11.5146864 | 4.42998334 | 11.601648 | 10.7522773 | 10.3129742 |
| Akt2 | 7.900821 | 1.74602406 | 4.64739684 | 7.50740455 | 6.69059007 | 7.16770014 |
| Akt3 | 0 | 7.96018226 | 0 | 7.35315614 | 2.17729258 | 7.80192128 |
| APC | 8.39335253 | 8.06797773 | 0 | 1.75142305 | 1.8927044 | 0 |
| Bad | 0 | 0 | 0 | 0 | 0 | 0 |
| Bax | 13.6760247 | 13.3176728 | 10.1228648 | 12.4537506 | 12.065459 | 11.730697 |
| Bcl11a | 0 | 0 | 0 | 0 | 2.71314006 | 0 |
| Bcl11b | 0 | 0 | 0 | 0 | 0 | 0 |
| Bc12 | 0 | 0 | 0 | 5.96566948 | 0 | 0 |
| Bcl2l1 | 8.42050716 | 8.5397273 | 8.24768464 | 7.86215744 | 7.9016606 | 7.95497919 |
| Bcl2l11 | 12.229686 | 10.1662961 | 8.73177655 | 9.85270326 | 8.51815048 | 10.7405021 |
| Bmi1 | 10.2387084 | 9.31396694 | 6.36310467 | 6.09634272 | 7.60876135 | 5.56419831 |
| Brd3 | 12.1884423 | 11.3821336 | 9.16931971 | 11.5847665 | 10.3403875 | 11.440292 |
| Casp8 | 9.45558864 | 9.60183486 | 0 | 8.96045034 | 9.60483639 | 9.81855927 |
| Casp9 | 10.983114 | 11.2997749 | 4.1377792 | 3.19674834 | 10.6397827 | 5.83890378 |
| Cbx2 | 3.91898214 | 6.93850275 | 2.68444976 | 5.77648185 | 4.81078818 | 5.84443483 |
| Cbx8 | 0 | 0 | 0 | 0 | 0 | 0 |
| Ccnc | 10.1627738 | 9.76965727 | 1.35355046 | 10.8327556 | 10.2251453 | 8.87207477 |
| Ccnd1 | 9.06711014 | 9.78697864 | 3.39014804 | 7.92433606 | 3.63100877 | 1.61547934 |
| Ccne2 | 10.0835116 | 11.319821 | 3.31015618 | 11.1288883 | 10.1332622 | 10.1575498 |
| CD34 | 0 | 0 | 0 | 0 | 2.43527288 | 0 |
| CD41 | 0 | 0 | 0 | 0 | 0 | 0 |
| CD48 | 7.9867099 | 4.18399936 | 9.74423407 | 0 | 4.67729616 | 10.3311682 |
| CD52 | 0 | 0 | 11.240447 | 0 | 0 | 0 |
| CD53 | 0 | 0 | 10.718297 | 0 | 0 | 3.47755329 |
| CD55 | 8.72944056 | 7.02423588 | 0.47752312 | 7.30323746 | 8.24842184 | 6.71445599 |
| CD63 | 0 | 0 | 0 | 0 | 0 | 8.48127545 |
| CD9 | 0 | 0 | 9.11541164 | 0 | 0 | 0 |
| Cdc42 | 14.5293724 | 14.6486438 | 12.7027151 | 14.5111753 | 13.888067 | 13.4366617 |
| Cdk1 | 13.1448472 | 11.9598095 | 4.7963663 | 12.1075135 | 9.74103341 | 9.76908148 |
| Cdk4 | 13.7494226 | 13.5258502 | 5.39347158 | 13.2132059 | 11.559049 | 12.3004943 |
| Cdkn2b | 0 | 7.45318318 | 2.03311382 | 0 | 0 | 0 |
| Cebpa | 0 | 0 | 1.93783554 | 4.65819114 | 0 | 5.14048555 |
| Csf1r | 0 | 0 | 1.18696511 | 7.77368454 | 0 | 4.57700679 |
| Ctnnb1 | 9.37096958 | 9.83654035 | 7.40506371 | 9.20900353 | 8.84284741 | 8.21343097 |
| Cycs | 15.3928254 | 15.4217364 | 9.00426594 | 14.9998511 | 14.3770315 | 13.6916743 |
| Dach1 | 10.0101828 | 8.37636957 | 1.87676967 | 8.46157503 | 8.37438306 | 10.2033785 |
| Dnmt1 | 13.5752055 | 13.5629832 | 0 | 13.1496695 | 12.9313015 | 12.2958028 |
| Dnmt3a | 10.9466601 | 8.51255715 | 2.35640024 | 11.4731999 | 8.29952651 | 10.4041202 |
| Dnmt3b | 8.48193549 | 10.1968081 | 0 | 0 | 8.25040799 | 9.82871253 |
| Dtx1 | 2.92606728 | 0 | 0 | 0 | 0 | 0 |
| Dtx4 | 0 | 7.82554643 | 4.92304422 | 0 | 0 | 0 |
| Ebf1 | 0 | 0 | 0 | 0 | 0 | 4.67430018 |
| Ep300 | 10.0634333 | 9.34942163 | 8.21967146 | 10.5762593 | 10.121413 | 8.87201287 |
| Epor | 7.16021518 | 6.62083976 | 4.26609394 | 6.51806867 | 5.52700029 | 6.09748073 |
| Erg | 0 | 0 | 0 | 0 | 0 | 5.68555987 |
| Esr1 | 0 | 0 | 0 | 0 | 0 | 2.51533896 |
| ETS1 | 3.76571695 | 0 | 13.1369479 | 0 | 0 | 6.54848086 |
| ETS2 | 5.16493701 | 9.76053928 | 3.59712435 | 8.2969717 | 8.32663081 | 0 |
| Etv3 | 5.60165087 | 4.73300606 | 0 | 3.87270615 | 3.64676519 | 5.18534646 |
| Etv6 | 10.2693757 | 10.3333581 | 3.69074449 | 10.0372574 | 8.50609787 | 9.04344531 |
| Ezh2 | 11.9461386 | 11.3539663 | 3.95203132 | 11.7590423 | 11.3908077 | 10.6007943 |
| Fas | 0 | 0 | 0 | 0 | 0 | 0 |
| Fcgr2b | 0 | 0 | 0 | 0 | 0 | 0.23425105 |
| Fcgr3 | 0 | 0 | 8.07918835 | 1.52130196 | 0 | 0 |
| Fli1 | 0 | 3.18894624 | 9.44502726 | 5.01815175 | 3.11865062 | 10.9846923 |
| Flt3 | 0 | 0 | 0 | 0 | 0 | 0 |
| Fosl1 | 3.90147912 | 9.28797126 | 0 | 8.52770733 | 0 | 8.71529532 |
| Foxo1 | 10.1059327 | 9.46825613 | 9.87277386 | 9.5068882 | 6.79088829 | 9.52716774 |
| Foxo3 | 11.880669 | 10.5592031 | 7.72876079 | 11.0684154 | 9.43154757 | 9.25402338 |
| Gapdh | 13.9931627 | 13.3857099 | 10.5346589 | 14.1082473 | 12.92779 | 11.113379 |
| Gata1 | 13.6190358 | 13.9835206 | 5.62359022 | 13.9713491 | 12.5041093 | 12.511952 |
| Gata2 | 3.92449227 | 2.84914463 | 0 | 1.01456852 | 0 | 6.83641959 |
| Gata3 | 0 | 2.25413244 | 7.84794279 | 0 | 0 | 0 |
| Gfi1 | 0 | 0 | 3.85434754 | 6.19324658 | 0.26378938 | 0 |
| Gfi1b | 13.6114909 | 13.5975417 | 5.17245225 | 13.9889482 | 13.039689 | 12.8606179 |
| Hes5 | 0 | 0 | 0 | 0 | 0 | 0 |
| Hey1 | 0 | 5.28516598 | 0 | 1.81677056 | 0 | 0 |
| Hlf | 0 | 0 | 0 | 0 | 0 | 9.91303577 |
| Id2 | 0 | 0 | 11.4964505 | 0 | 0 | 0 |
| Ifi203 | 0 | 0 | 11.6357055 | 0 | 7.02168865 | 6.48869747 |
| Ifi205 | 0 | 0.48785466 | 0 | 0 | 0 | 0.04446014 |
| Ifitm1 | 0 | 0 | 0 | 3.39877768 | 0 | 0 |
| Ikzf1 | 14.3275555 | 14.0316022 | 9.42911846 | 13.1119643 | 12.6313804 | 12.3162902 |
| Ikzf2 | 5.2858744 | 0 | 0 | 0 | 4.28650806 | 10.7927559 |
| Il7R | 0 | 0 | 0 | 0 | 0 | 0 |
| Irf4 | 0 | 0 | 0 | 0 | 0 | 3.75675557 |
| Irf6 | 4.47123029 | 3.22821179 | 4.339607 | 1.36832553 | 0 | 0 |
| Irf8 | 3.37099886 | 2.31508294 | 0 | 6.44379002 | 0 | 0 |
| Kdr | 0 | 0 | 0 | 0 | 0 | 0 |
| Kit | 13.3537238 | 12.8580221 | 11.7556361 | 11.3366407 | 12.6638144 | 13.3561223 |
| Klf1 | 14.2979136 | 13.6308232 | 5.96653235 | 13.5935874 | 12.7713044 | 12.127983 |
| Klf12 | 3.98365645 | 0 | 0 | 0 | 0 | 0 |
| Ldb1 | 14.5635445 | 14.0908295 | 8.35195984 | 14.3971418 | 13.093488 | 13.9731006 |
| Lin28a | 4.24471852 | 6.991793 | 0 | 6.53901741 | 3.59537839 | 7.3031128 |
| Lmo2 | 12.9830675 | 12.5177653 | 4.67072022 | 12.0390668 | 11.5425664 | 10.1528281 |
| Ly6a | 0 | 0 | 0 | 0 | 0 | 0 |
| Lyl1 | 8.76148245 | 9.87461972 | 0 | 8.97801904 | 1.61425898 | 0 |
| Mbd2 | 14.3088686 | 14.1788793 | 10.6850909 | 13.7604538 | 13.6786771 | 12.4484023 |
| Meis1 | 0 | 0 | 0 | 0 | 0 | 0 |
| Mllt3 | 6.75437066 | 4.87929073 | 0.36628079 | 7.04238465 | 1.04227285 | 0 |
| Mpl | 0 | 0 | 0 | 0 | 7.82872486 | 0 |
| Muc13 | 6.50258482 | 9.17003155 | 2.94436572 | 6.93025842 | 0 | 8.96811757 |
| Myb | 14.5775089 | 14.3566219 | 6.22959734 | 13.1602938 | 13.678872 | 15.059386 |
| Myc | 14.0547532 | 13.3673906 | 5.6369054 | 14.4815067 | 12.1006266 | 13.6012191 |
| Mycn | 0 | 0 | 5.97826269 | 0 | 0 | 0 |
| Ndn | 0 | 0 | 0 | 3.30758821 | 0 | 4.50321309 |
| Nfat5 | 6.8997464 | 8.60819523 | 8.05163374 | 9.06664427 | 5.40897018 | 6.6641746 |
| Nfia | 14.4761658 | 14.140814 | 5.89547833 | 14.0665307 | 13.4354033 | 12.1410189 |
| Nfkb1 | 5.0181859 | 2.309416 | 3.98387116 | 4.10351957 | 4.95579258 | 0 |
| Notch1 | 0 | 0 | 0 | 0 | 0 | 0 |
| Pax4 | 0 | 0 | 0 | 0 | 0 | 0 |
| Pax5 | 0 | 0 | 0 | 0 | 0 | 0.97924165 |
| Pax9 | 0 | 0 | 0 | 5.83889268 | 0 | 0 |
| Pbx1 | 0 | 0 | 0 | 0 | 0 | 0 |
| PIk3ca | 11.0496351 | 11.3740226 | 8.25771166 | 9.9484021 | 8.3447194 | 0 |
| PIk3R2 | 13.0336384 | 11.9086806 | 4.45143877 | 12.6406896 | 12.5663171 | 10.6748968 |
| Plag1 | 0 | 0 | 0 | 8.62527154 | 8.27129457 | 0 |
| Prf1 | 0 | 0 | 7.95861297 | 0 | 5.64429588 | 0 |
| Pten | 10.8777303 | 10.2906901 | 8.05284894 | 10.7251068 | 10.0886075 | 10.5809166 |
| Rb1 | 13.3531695 | 12.8920186 | 8.68520402 | 12.4552038 | 12.5020608 | 11.9804354 |
| Rora | 0 | 0 | 0 | 0 | 0 | 0 |
| Runx1 | 10.3327309 | 10.1695965 | 2.15987744 | 8.22784853 | 8.33584994 | 8.47230462 |
| Runx2 | 4.49773995 | 0 | 0 | 3.58458245 | 0 | 0 |
| Satb1 | 0 | 0 | 8.06753085 | 0 | 0 | 2.71209422 |
| Sdpr | 0 | 0.18954202 | 0 | 0 | 0 | 0 |
| Sell | 0 | 7.67225672 | 9.98123205 | 0 | 0 | 0 |
| Sfpi1 | 0 | 0 | 0 | 0.46212458 | 0 | 8.14790234 |
| Slamf1 | 0 | 0 | 0 | 0 | 0 | 0 |
| Smarca4 | 14.0639422 | 13.68311 | 5.6018656 | 13.0990363 | 13.3134848 | 12.9739128 |
| Sos1 | 9.61043591 | 9.24302377 | 0.84637826 | 9.30907613 | 8.92422084 | 9.07163316 |
| Stat1 | 5.06501781 | 7.57488158 | 2.81371618 | 2.88085862 | 1.86966235 | 1.68793237 |
| Stat3 | 6.91429151 | 7.407193 | 8.67732684 | 7.75419499 | 8.30889661 | 10.0828057 |
| Stat4 | 0 | 0 | 8.79727168 | 0 | 1.92039891 | 0 |
| Stat6 | 10.7036377 | 10.9195918 | 7.52472475 | 9.55035514 | 8.28177793 | 10.9191881 |
| Suz12 | 13.538197 | 13.0876648 | 8.23876575 | 13.2750979 | 12.6890707 | 11.8669775 |
| Tal1 | 5.4291009 | 6.69589556 | 0 | 7.56623027 | 4.77147026 | 4.69263937 |
| Tcf3 | 10.9470082 | 10.4293296 | 0 | 10.598365 | 9.91291781 | 2.59346866 |
| Tcf4 | 11.9008105 | 11.8187116 | 7.59295834 | 11.3626835 | 10.2619576 | 10.2854661 |
| Tcf7 | 0 | 0.93440846 | 6.96284694 | 0 | 0 | 0 |
| Tek | 5.48770868 | 0 | 0 | 7.34302092 | 0.55382256 | 0 |
| Tfrc | 15.1003637 | 14.1185956 | 6.68189103 | 13.6781033 | 14.0561821 | 11.5817117 |
| Tgfb1 | 0 | 5.9321715 | 3.29941964 | 6.77315808 | 8.10258704 | 0 |
| Tgfb2 | 0 | 0 | 0 | 0 | 0 | 0 |
| Tgfb3 | 0 | 0 | 0 | 8.05286418 | 2.7676147 | 0 |
| Tnfrsf1a | 0 | 0 | 9.44208849 | 0 | 0 | 7.52151547 |
| Tnfrsf1b | 0 | 7.33777528 | 8.96218157 | 0 | 4.27782113 | 0 |
| Tnfrsf21 | 0 | 5.97030087 | 1.15224807 | 4.81031941 | 3.82725759 | 5.22821771 |
| Tnfsf10 | 0 | 0 | 7.50776293 | 6.28059236 | 0 | 0.19985742 |
| Tnfsf12 | 0 | 0 | 0 | 0 | 0 | 0 |
| Tob1 | 7.49341793 | 9.775516 | 4.53888952 | 7.30658141 | 5.12736672 | 0 |
| vWF | 4.76480777 | 5.8950733 | 0 | 0 | 0 | 0 |
| Zbtb20 | 7.31711148 | 6.7543605 | 6.09700763 | 0 | 0 | 3.99093784 |
| Zbtb38 | 10.5208922 | 8.98561327 | 8.24763973 | 10.833188 | 9.72578991 | 9.49544458 |
| Zfp532 | 0 | 0 | 0 | 0 | 0 | 0 |
| Zfp612 | 2.66699095 | 0 | 5.65730748 | 3.15028498 | 0 | 0 |
| Zfpm1 | 9.91367246 | 8.38424838 | 0 | 7.63005335 | 5.88946627 | 8.04762332 |
| Zhx2 | 8.58545196 | 0 | 6.98674013 | 9.3468461 | 5.30923464 | 9.33665392 |

TABLE 6-11
Single cell expression data (reduced list)—Control

| Factor | MPP1 | MPP2 | MPP3 | MPP4 | MPP5 | MPP6 |
|---|---|---|---|---|---|---|
| Actb | 15.9338457 | 15.4232208 | 16.2711873 | 14.6823 | 14.2918152 | 15.8659118 |
| Aebp2 | 7.21100476 | 5.2867401 | 6.93025793 | 5.90925673 | 5.25462477 | 9.18427092 |
| Ahr | 0 | 0 | 8.34801326 | 0 | 0 | 0 |
| Akt1 | 10.720231 | 9.40876898 | 11.0220046 | 9.04411511 | 9.0996424 | 11.2217446 |
| Akt2 | 2.21487307 | 5.4868309 | 0 | 0 | 5.35510644 | 0 |
| Akt3 | 8.87303458 | 8.64995993 | 8.90809022 | 8.03436457 | 0 | 10.0275887 |
| APC | 9.11114608 | 0 | 8.0871966 | 1.98598274 | 8.73132197 | 4.78295182 |
| Bad | 0 | 0 | 0 | 0 | 0 | 8.89131665 |
| Bax | 8.98329445 | 10.498022 | 9.02157645 | 9.45119586 | 0 | 9.14566934 |
| Bcl11a | 0 | 0 | 0 | 8.89978638 | 0 | 8.82676654 |
| Bcl11b | 0 | 0 | 0 | 0 | 0 | 0 |
| Bcl2 | 5.4456877 | 6.76850037 | 8.56326925 | 0 | 0 | 6.41872246 |
| Bcl2l1 | 8.77442328 | 9.4903021 | 8.32482213 | 8.37825811 | 0 | 9.68984903 |
| Bcl2l11 | 8.65261883 | 0 | 8.55329576 | 0 | 0 | 0 |
| Bmi1 | 7.92005647 | 8.96348283 | 7.6988806 | 5.99607904 | 8.09101102 | 10.2547476 |
| Brd3 | 11.0992941 | 10.6513546 | 9.61291134 | 9.43861553 | 6.3757271 | 10.8237539 |
| Casp8 | 11.3348993 | 11.0515753 | 10.9825524 | 9.29875931 | 8.5871616 | 10.8985747 |
| Casp9 | 8.73428375 | 10.0497654 | 0 | 0 | 0 | 2.43946663 |
| Cbx2 | 7.57992406 | 6.71714066 | 0 | 0 | 0 | 0.6708544 |
| Cbx8 | 0 | 0 | 0 | 0 | 0 | 0 |
| Ccnc | 8.35164492 | 0 | 6.07511496 | 9.13555725 | 0 | 7.33770601 |
| Ccnd1 | 8.60823223 | 0 | 9.93021361 | 0 | 9.80132789 | 8.95924036 |
| Ccne2 | 7.6057764 | 10.4324496 | 10.1697513 | 7.75985448 | 0 | 10.6399418 |
| CD34 | 11.1537947 | 12.1750274 | 11.4199898 | 10.0501247 | 10.5540352 | 11.3151543 |
| CD41 | 0 | 5.28178356 | 0 | 0 | 0 | 0 |
| CD48 | 9.48857003 | 11.0978106 | 11.3892976 | 8.80517983 | 0 | 9.56184962 |
| CD52 | 9.67070973 | 9.66597181 | 10.8936843 | 7.05264794 | 7.44343937 | 10.2105126 |
| CD53 | 11.1467937 | 11.241697 | 10.1035022 | 11.4194355 | 0 | 11.4433546 |
| CD55 | 0 | 0 | 0 | 0 | 0 | 0 |
| CD63 | 6.93667918 | 10.6830361 | 7.91059718 | 7.48471238 | 4.0814483 | 0 |
| CD9 | 0 | 0 | 0 | 9.13917551 | 0 | 0 |
| Cdc42 | 13.4222253 | 12.5348596 | 13.56969 | 12.3378718 | 11.7636509 | 12.8887671 |
| Cdk1 | 10.9643801 | 11.4007291 | 9.70754751 | 0 | 0 | 10.4661432 |
| Cdk4 | 11.8074379 | 10.3164272 | 12.5018024 | 9.48804452 | 6.81583478 | 11.7800185 |
| Cdkn2b | 0 | 0 | 2.77346992 | 0 | 0 | 0 |
| Cebpa | 9.22772932 | 10.0275028 | 11.2952199 | 11.0642013 | 9.09418965 | 10.4493234 |
| Csf1r | 0 | 8.45310432 | 8.99182682 | 7.91613811 | 10.0723015 | 0 |
| Ctnnb1 | 8.32067527 | 5.00574303 | 8.39061689 | 8.19898063 | 4.79592084 | 8.46222031 |
| Cycs | 13.0347923 | 12.4656213 | 14.3162078 | 9.98439188 | 9.65986044 | 12.6497946 |
| Dach1 | 0 | 13.3892767 | 0 | 7.3947807 | 9.10470453 | 0 |
| Dnmt1 | 12.8259216 | 12.6055461 | 12.7124172 | 10.1043631 | 0 | 12.0574902 |
| Dnmt3a | 11.5381376 | 7.80820219 | 11.1160495 | 10.4359516 | 9.17576912 | 10.796858 |
| Dnmt3b | 10.7508563 | 11.1492963 | 9.71848489 | 10.1049899 | 8.03011401 | 10.8681675 |
| Dtx1 | 0 | 0 | 0.31107154 | 0 | 0 | 0 |
| Dtx4 | 11.1069971 | 7.43011153 | 12.4091038 | 0 | 0 | 0 |
| Ebf1 | 0 | 0 | 0 | 0 | 0 | 0 |
| Ep300 | 8.75076257 | 8.59075653 | 9.62468843 | 9.68032474 | 9.58816102 | 8.39625294 |
| Epor | 4.91252317 | 3.1681373 | 2.6614969 | 0 | 3.60216649 | 0 |
| Erg | 9.15107944 | 12.1140199 | 10.0602319 | 8.05974652 | 9.1838276 | 7.70552462 |
| Esr1 | 11.9774405 | 8.93512079 | 9.30574164 | 10.8765411 | 0 | 10.0872926 |
| ETS1 | 10.5968066 | 10.5087649 | 0 | 8.1786786 | 0 | 10.9157853 |
| ETS2 | 8.80623923 | 5.91625835 | 6.07444663 | 8.44682963 | 1.07952469 | 8.81372256 |
| Etv3 | 5.042175 | 0 | 6.17334389 | 5.48927278 | 0 | 4.47456273 |
| Etv6 | 11.2690271 | 11.8468993 | 10.1410346 | 10.3082532 | 10.7932873 | 11.5654449 |
| Ezh2 | 10.9805883 | 10.1182621 | 9.56833692 | 8.93691074 | 5.75295828 | 11.0075626 |
| Fas | 0 | 0 | 0 | 0 | 0 | 0 |
| Fcgr2b | 0 | 0 | 7.59523747 | 0 | 0 | 0 |
| Fcgr3 | 0 | 0 | 0 | 0 | 0 | 0 |
| Fli1 | 10.0608425 | 11.8155209 | 11.4638535 | 11.1403327 | 11.1245373 | 11.1412932 |
| Flt3 | 13.4713208 | 10.9848512 | 12.2344582 | 12.3865902 | 11.8521808 | 13.8892265 |
| Fosl1 | 9.53355426 | 0 | 0 | 0 | 0 | 0 |
| Foxo1 | 10.3135469 | 10.291036 | 8.38726315 | 8.71085607 | 9.18316568 | 10.070319 |
| Foxo3 | 8.99316696 | 10.5108484 | 7.44733165 | 8.87606497 | 8.84685185 | 10.0201581 |
| Gapdh | 11.6345343 | 12.8310252 | 11.770259 | 9.91293629 | 9.88727626 | 10.8114969 |
| Gata1 | 0 | 0 | 0 | 5.30872103 | 0 | 0 |
| Gata2 | 0 | 6.25587723 | 0 | 0 | 0 | 5.46348074 |
| Gata3 | 8.22743301 | 6.73112619 | 0 | 7.17096276 | 7.71368531 | 0 |
| Gfi1 | 7.2915492 | 8.85268611 | 0 | 0 | 8.61482142 | 8.29984645 |
| Gfi1b | 0 | 10.9458698 | 7.94961583 | 0 | 0 | 9.2539235 |
| Hes5 | 0 | 0 | 0 | 0 | 0 | 0 |
| Hey1 | 0 | 0 | 0 | 0 | 0 | 0 |
| Hlf | 11.6107089 | 11.3506889 | 8.23973525 | 11.1220263 | 12.3616717 | 9.67605941 |
| Id2 | 0 | 0 | 0 | 3.01634284 | 0 | 0 |
| Ifi203 | 12.2933836 | 13.4311987 | 12.3810812 | 11.2387169 | 11.1057026 | 13.854702 |
| Ifi205 | 1.46825299 | 0 | 0 | 0 | 0 | 0 |
| Ifitm1 | 10.3158671 | 12.2548019 | 1.80612474 | 9.53491036 | 11.6097006 | 10.8254147 |
| Ikzf1 | 12.2449774 | 10.9137692 | 11.8885062 | 9.44640906 | 10.0772827 | 11.7883861 |
| Ikzf2 | 8.72612533 | 10.5247685 | 0 | 9.54996851 | 9.19777951 | 0 |
| Il7R | 0 | 0 | 0 | 0 | 0 | 0 |
| Irf4 | 8.88932449 | 0 | 0 | 0 | 0 | 0 |
| Irf6 | 0 | 2.28451212 | 3.12345728 | 0 | 2.94453997 | 7.03646954 |
| Irf8 | 10.1140031 | 9.69303494 | 12.6201361 | 0 | 0 | 7.96421851 |
| Kdr | 0 | 0 | 0 | 0 | 0 | 0 |
| Kit | 10.3323157 | 13.290097 | 10.3759975 | 11.6752403 | 11.4546255 | 9.73802765 |
| Klf1 | 0 | 0 | 0 | 0 | 0 | 0 |
| K1f12 | 0 | 7.5771355 | 0 | 0 | 0 | 0 |
| Ldb1 | 11.7920145 | 11.5152136 | 11.7984669 | 10.9682206 | 10.6778344 | 11.0742708 |
| Lin28a | 7.77696226 | 2.72948667 | 3.67493945 | 7.0652472 | 9.08749361 | 0 |
| Lmo2 | 10.6837852 | 10.8046961 | 10.6266379 | 10.9303176 | 10.719542 | 11.3314271 |
| Ly6a | 11.5474621 | 10.2394989 | 7.73593565 | 8.32586298 | 10.6491694 | 9.60404877 |
| Lyl1 | 8.11242278 | 0 | 10.1020158 | 0 | 0 | 8.83335686 |
| Mbd2 | 10.0753161 | 10.3506985 | 11.4298385 | 9.11525309 | 10.5529714 | 10.6747769 |
| Meis1 | 8.19052316 | 9.67159559 | 6.53658539 | 0 | 9.3704605 | 8.58771639 |
| Mllt3 | 0 | 7.01032363 | 0 | 0 | 0 | 0 |
| Mpl | 0 | 11.6188289 | 0 | 0 | 0 | 0 |
| Muc13 | 9.18176099 | 10.7974567 | 0 | 10.159424 | 0 | 0 |
| Myb | 13.8600806 | 13.3102917 | 13.8966992 | 11.8930023 | 12.3157054 | 12.0374473 |
| Myc | 12.4572692 | 12.7756443 | 13.0591357 | 10.6719002 | 0 | 11.658761 |
| Mycn | 4.48759571 | 12.0648986 | 7.88422472 | 10.1149438 | 10.4601304 | 12.3658929 |
| Ndn | 0 | 8.73723256 | 0 | 0 | 0 | 9.05565846 |
| Nfat5 | 9.23389178 | 8.43831408 | 5.08317626 | 7.08881328 | 7.14783983 | 9.11031201 |
| Nfia | 8.22536613 | 10.3181464 | 0 | 0 | 0 | 0 |
| Nfkb1 | 0 | 4.25773876 | 6.36053701 | 6.28335202 | 0 | 0 |
| Notch1 | 10.5411213 | 0 | 10.0596251 | 9.15986762 | 7.79669562 | 10.5613034 |
| Pax4 | 0 | 0 | 0 | 0 | 0 | 0 |
| Pax5 | 0 | 0 | 0 | 0 | 0 | 0 |
| Pax9 | 0 | 4.39855399 | 0 | 0 | 7.47812976 | 0 |
| Pbx1 | 0 | 0 | 3.98886737 | 0 | 0 | 0 |
| PIk3ca | 9.98779925 | 8.66693277 | 8.36771159 | 8.07241137 | 8.11823529 | 10.7576739 |
| PIk3R2 | 0 | 8.41914228 | 11.2909208 | 9.22643964 | 0 | 10.8038558 |
| Plag1 | 8.91111651 | 0 | 0 | 0 | 0 | 0 |
| Prf1 | 0 | 0 | 0 | 0 | 0 | 0 |
| Pten | 9.73746823 | 8.52844961 | 10.3610336 | 9.7554549 | 8.9576986 | 9.39466925 |
| Rb1 | 10.56726 | 11.135636 | 10.6281849 | 9.77464462 | 9.64300093 | 8.52928352 |
| Rora | 0 | 0 | 0 | 0 | 8.95379549 | 0 |
| Runx1 | 0 | 7.57506234 | 11.3878361 | 9.64485117 | 0 | 8.01381659 |
| Runx2 | 8.01374944 | 6.92871391 | 8.2119077 | 5.88438904 | 6.41322446 | 7.86904824 |
| Satb1 | 12.19321 | 9.07418197 | 12.4338909 | 11.4331637 | 8.41406481 | 11.7354143 |
| Sdpr | 0 | 0 | 0 | 0 | 0 | 0 |
| Sell | 0 | 11.4107566 | 11.5576376 | 0 | 0 | 9.39436203 |
| Sfpi1 | 10.4751592 | 10.2858722 | 10.7956608 | 10.7612319 | 8.28792429 | 9.65907002 |
| Slamf1 | 0 | 0 | 0 | 0 | 0 | 0 |
| Smarca4 | 13.0055606 | 11.8353641 | 12.3437472 | 11.4881732 | 11.951662 | 11.9434175 |
| Sos1 | 2.66805577 | 7.04399519 | 7.54270055 | 7.21891711 | 7.15270243 | 6.4810902 |
| Stat1 | 3.79515281 | 3.7506045 | 2.42604397 | 7.25585273 | 2.19651358 | 5.20779754 |
| Stat3 | 0 | 7.35454462 | 0 | 11.4070872 | 9.90639954 | 0 |
| Stat4 | 8.61934296 | 8.48689909 | 0 | 7.19354193 | 6.92208828 | 8.11912795 |
| Stat6 | 8.38514449 | 9.97948225 | 10.91246 | 10.0950254 | 10.3501987 | 11.7262422 |
| Suz12 | 12.1314037 | 11.5904284 | 12.2840569 | 10.4055215 | 6.87697186 | 12.3236445 |
| Tal1 | 0 | 5.08496506 | 2.73114378 | 0 | 7.48698209 | 0 |
| Tcf3 | 6.67565349 | 8.31215232 | 2.15386392 | 0 | 0 | 8.29747218 |
| Tcf4 | 11.1250971 | 9.46376933 | 11.7038871 | 11.1179238 | 9.78786393 | 11.7491444 |
| Tcf7 | 0 | 0 | 0 | 0 | 0 | 0 |
| Tek | 0 | 9.20065767 | 0 | 0 | 0 | 0 |
| Tfrc | 11.1514151 | 9.64401783 | 12.0859674 | 9.26164167 | 0 | 10.1615771 |
| Tgfb1 | 0 | 0 | 7.66944594 | 0 | 0 | 7.04752663 |
| Tgfb2 | 0 | 0 | 0 | 0 | 0 | 0 |
| Tgfb3 | 0 | 0 | 0 | 0 | 0 | 0 |
| Tnfrsf1a | 10.4658528 | 3.19243814 | 1.74940044 | 9.34788925 | 9.69155039 | 8.79792759 |
| Tnfrsf1b | 9.13020949 | 8.49093408 | 9.13944664 | 0 | 5.57625399 | 0 |
| Tnfrsf2l | 6.50412724 | 7.08061356 | 6.13813065 | 6.59804131 | 5.91417667 | 6.16646549 |
| Tnfsf10 | 0.19476122 | 0 | 0 | 0 | 6.29693535 | 7.28794504 |
| Tnfsf12 | 0 | 0 | 0 | 0 | 0 | 0 |
| Tob1 | 0 | 0 | 0 | 5.53534788 | 6.34080934 | 0 |
| vWF | 0 | 0 | 0 | 0 | 0 | 0 |
| Zbtb20 | 7.26349348 | 7.01562217 | 0 | 0 | 7.96980448 | 9.33850376 |
| Zbtb38 | 6.57611355 | 10.1472497 | 8.96566992 | 7.6851333 | 8.72294732 | 7.63005742 |
| Zfp532 | 3.74373752 | 0 | 0 | 0 | 0 | 0 |
| Zfp612 | 0 | 7.24390911 | 0 | 0 | 5.76142983 | 0 |
| Zfpm1 | 4.4433929 | 4.77658905 | 4.37031599 | 6.57885334 | 4.4872645 | 5.50679362 |
| Zhx2 | 0 | 0 | 0 | 0 | 0 | 0 |

TABLE 6-12
Single cell expression data (reduced list)-Control

| Factor | MPP7 | MPP8 | MPP9 | MPP10 |
|---|---|---|---|---|
| Actb | 15.5799561 | 16.4231342 | 14.9413529 | 16.4806567 |
| Aebp2 | 4.74041619 | 5.79768478 | 4.60544211 | 5.70833163 |
| Ahr | 0 | 0 | 0 | 0 |
| Akt1 | 8.28402993 | 10.5440223 | 8.94826142 | 10.0634546 |
| Akt2 | 0 | 0 | 0 | 5.73559526 |
| Akt3 | 9.55466835 | 7.76861222 | 7.22498152 | 9.10794373 |
| APC | 0 | 0 | 3.09166097 | 7.5684068 |
| Bad | 0 | 0 | 0 | 0 |
| Bax | 7.56956863 | 10.0339298 | 9.18437556 | 10.0584079 |
| Bcl11a | 0 | 8.73822897 | 0 | 0 |
| Bcl11b | 0 | 0 | 0 | 0 |
| Bcl2 | 0 | 6.49304714 | 0 | 0 |
| Bcl2l1 | 0 | 9.34594529 | 0 | 9.25723428 |
| Bcl2l11 | 0 | 8.61453833 | 8.08425995 | 9.37832844 |
| Bmi1 | 0 | 8.32976055 | 8.25021212 | 7.88080894 |
| Brd3 | 9.25530682 | 11.2819662 | 8.12620738 | 10.4587875 |
| Casp8 | 10.6378139 | 11.1806726 | 10.2895215 | 11.39495 |
| Casp9 | 0 | 0 | 0 | 0.01340377 |
| Cbx2 | 6.79558984 | 8.59803667 | 7.0009243 | 8.51339363 |
| Cbx8 | 0 | 0 | 0 | 0 |
| Ccnc | 0 | 9.44435886 | 0 | 9.60093989 |
| Ccnd1 | 0 | 9.91482334 | 8.70488465 | 9.74960081 |
| Ccne2 | 8.92637293 | 10.6434763 | 0 | 11.3631899 |
| CD34 | 10.913548 | 11.4119115 | 10.4402497 | 11.0324695 |
| CD41 | 0 | 8.7488255 | 0 | 0 |
| CD48 | 9.32813788 | 11.6576097 | 3.44806841 | 10.543773 |
| CD52 | 9.61936432 | 8.73437329 | 8.14742149 | 10.7452684 |
| CD53 | 11.2776098 | 11.1779516 | 9.29476445 | 11.5298596 |
| CD55 | 0 | 0 | 0 | 0 |
| CD63 | 0 | 7.40996888 | 7.37062015 | 0 |
| CD9 | 0 | 0 | 0 | 0 |
| Cdc42 | 13.2349309 | 13.2478421 | 11.8304766 | 13.479261 |
| Cdk1 | 3.55141534 | 10.9443851 | 7.01908412 | 0 |
| Cdk4 | 10.9902569 | 12.2074899 | 10.5113998 | 11.5285061 |
| Cdkn2b | 0 | 0 | 0 | 0 |
| Cebpa | 9.95273834 | 9.17238843 | 9.49773051 | 10.1225975 |
| Csf1r | 9.04324667 | 0.80192533 | 7.08749126 | 10.0144786 |
| Ctnnb1 | 5.98292685 | 8.32930377 | 7.13625717 | 8.62467592 |
| Cycs | 11.4299521 | 13.7723272 | 10.3832219 | 13.3171266 |
| Dach1 | 0 | 0 | 0 | 0 |
| Dnmt1 | 11.4993032 | 13.0255564 | 11.8407164 | 12.2146515 |
| Dnmt3a | 10.7606522 | 10.2684942 | 8.39313135 | 11.1114075 |
| Dnmt3b | 7.92873762 | 10.0182754 | 10.504402 | 10.6600185 |
| Dtx1 | 0 | 4.7728077 | 0 | 0 |
| Dtx4 | 11.6922822 | 0 | 0 | 10.7071 |
| Ebf1 | 0 | 0 | 0 | 0 |
| Ep300 | 9.37108183 | 9.81215211 | 7.97358115 | 9.71578584 |
| Epor | 4.28849905 | 5.02706212 | 5.93378806 | 2.52024536 |
| Erg | 11.0872096 | 10.9417369 | 12.2901755 | 10.555884 |
| Esr1 | 11.6554563 | 8.57331038 | 9.75278719 | 10.1047334 |
| ETS1 | 11.9723848 | 3.46036603 | 3.47419373 | 11.9193271 |
| ETS2 | 8.55484649 | 0 | 0 | 1.48502139 |
| Etv3 | 6.2991095 | 3.863445 | 5.31929463 | 3.86067028 |
| Etv6 | 9.37003784 | 11.6844245 | 12.2546514 | 11.8922382 |
| Ezh2 | 8.86828602 | 10.9599669 | 8.96305659 | 10.2990075 |
| Fas | 0 | 0 | 0 | 0 |
| Fcgr2b | 0 | 6.76599483 | 0 | 5.22192452 |
| Fcgr3 | 0 | 0 | 0 | 0 |
| Fli1 | 11.8629795 | 11.3479034 | 10.9761779 | 11.4859541 |
| Flt3 | 13.2221419 | 13.2364137 | 12.1561632 | 13.2561177 |
| Fosl1 | 0 | 0 | 0 | 0 |
| Foxo1 | 10.1123954 | 10.2137253 | 10.6020045 | 10.7842805 |
| Foxo3 | 5.59015224 | 9.10555731 | 11.3588563 | 10.8953427 |
| Gapdh | 11.0477044 | 13.1655109 | 11.4008552 | 13.1404894 |
| Gata1 | 0.94564173 | 0 | 0 | 0 |
| Gata2 | 3.65013785 | 3.51256127 | 5.2315933 | 0 |
| Gata3 | 1.39088214 | 6.3891916 | 0 | 0 |
| Gfi1 | 8.84634557 | 6.27872603 | 6.48893372 | 0 |
| Gfi1b | 0 | 8.82811597 | 10.2832164 | 9.20982917 |
| Hes5 | 4.64107681 | 0 | 6.957973 | 0 |
| Hey1 | 0 | 0 | 0 | 0 |
| Hlf | 0 | 9.33667569 | 11.9867391 | 9.23572861 |
| Id2 | 0 | 8.21997193 | 0 | 4.79088829 |
| Ifi203 | 13.6649212 | 12.9454442 | 12.6378994 | 13.2045669 |
| Ifi205 | 0 | 0 | 0 | 0 |
| Ifitm1 | 0 | 11.8326933 | 9.99492608 | 8.92490776 |
| Ikzf1 | 12.3564729 | 11.2597407 | 11.3032006 | 10.227332 |
| Ikzf2 | 10.0349533 | 9.92808204 | 8.40270492 | 0 |
| Il7R | 8.47052626 | 0 | 0 | 4.98728556 |
| Irf4 | 0 | 0 | 0 | 0 |
| Irf6 | 0 | 0 | 0 | 0 |
| Irf8 | 8.35824062 | 10.9054686 | 0 | 11.9512698 |
| Kdr | 0 | 0 | 0 | 0 |
| Kit | 10.6608131 | 11.4159407 | 11.8825308 | 10.6648273 |
| Klf1 | 0 | 6.3799233 | 0 | 0 |
| Klf12 | 0 | 0 | 0 | 0 |
| Ldb1 | 11.0037537 | 10.768767 | 11.3221586 | 11.5395242 |
| Lin28a | 0 | 0 | 8.71671372 | 8.10538829 |
| Lmo2 | 9.8811249 | 11.1975103 | 11.572644 | 10.6241439 |
| Ly6a | 10.5512136 | 8.03714344 | 10.4350633 | 10.3456629 |
| Lyl1 | 9.23026917 | 7.59193214 | 0 | 9.49619226 |
| Mbd2 | 11.6682738 | 11.3388742 | 10.0098962 | 10.6943395 |
| Meis1 | 6.79368245 | 8.42564079 | 8.98994745 | 7.80483069 |
| Mllt3 | 0 | 0 | 0 | 0 |
| Mpl | 0 | 0 | 9.17845367 | 0 |
| Muc13 | 0 | 9.30164297 | 7.01923521 | 0 |
| Myb | 13.4971968 | 13.3599043 | 12.082765 | 13.8765431 |
| Myc | 11.6030817 | 12.0932166 | 8.10743215 | 11.4606205 |
| Mycn | 8.2487794 | 0 | 10.0709306 | 0 |
| Ndn | 0 | 0 | 10.0775359 | 0 |
| Nfat5 | 7.09690528 | 8.60254985 | 7.31614621 | 7.43938448 |
| Nfia | 0 | 0 | 10.937255 | 0 |
| Nfkb1 | 3.83053939 | 4.11240597 | 5.24127431 | 3.64341386 |
| Notch1 | 11.1593775 | 8.27953256 | 7.48014451 | 9.14338513 |
| Pax4 | 0 | 0 | 0 | 0 |
| Pax5 | 0 | 0 | 0 | 0 |
| Pax9 | 0 | 0 | 0 | 0 |
| Pbx1 | 0 | 4.9309508 | 0 | 0 |
| PIk3ca | 8.96893649 | 10.6627449 | 8.54724566 | 9.22944916 |
| PIk3R2 | 8.65643169 | 11.8510785 | 10.1724212 | 0 |
| Plag1 | 0 | 0 | 0 | 0 |
| Prf1 | 0 | 0 | 0 | 0 |
| Pten | 8.25469691 | 9.67626184 | 6.97446432 | 9.5307241 |
| Rb1 | 9.59233164 | 11.5007352 | 9.77688089 | 11.1455471 |
| Rora | 0 | 0 | 8.65726707 | 0 |
| Runx1 | 0 | 10.0522268 | 8.31416339 | 9.56394879 |
| Runx2 | 7.48955293 | 5.94137868 | 5.7987657 | 7.28443718 |
| Satb1 | 12.4017526 | 9.90535075 | 7.60722496 | 12.7657794 |
| Sdpr | 0 | 0 | 0 | 0 |
| Sell | 11.5341189 | 11.4001825 | 9.51107337 | 9.17958828 |
| Sfpi1 | 9.55284835 | 10.8357053 | 8.89491205 | 10.3685731 |
| Slamf1 | 0 | 0 | 0 | 0 |
| Smarca4 | 12.3428509 | 13.5642625 | 10.6464189 | 11.9223443 |
| Sos1 | 8.19189077 | 0 | 7.3490338 | 8.77541216 |
| Stat1 | 4.94305767 | 3.60841055 | 0 | 6.83329035 |
| Stat3 | 9.23352711 | 10.6650348 | 11.2676229 | 0 |
| Stat4 | 9.73904725 | 9.11900076 | 8.47015672 | 7.05959532 |
| Stat6 | 9.78343857 | 10.2042159 | 9.87121731 | 10.0443104 |
| Suz12 | 10.3249963 | 12.0359278 | 10.0398783 | 11.7614625 |
| Tal1 | 0 | 0 | 8.600419 | 7.76085711 |
| Tcf3 | 8.03699653 | 5.45181491 | 9.06930734 | 0 |
| Tcf4 | 11.8413493 | 11.1111843 | 9.69541167 | 12.2817037 |
| Tcf7 | 0 | 0 | 0 | 6.54941349 |
| Tek | 0 | 0 | 0 | 0 |
| Tfrc | 10.6830 | 9029.59395121 | 0 | 10.8582641 |
| Tgfb1 | 0 | 8.23296021 | 0 | 0 |
| Tgfb2 | 0 | 0 | 0 | 0 |
| Tgfb3 | 0 | 0 | 0 | 0 |
| Tnfrsf1a | 8.68589512 | 10.7994818 | 0 | 9.35978037 |
| Tnfrsf1b | 7.9316098 | 8.07814768 | 0 | 2.48402645 |
| Tnfrsf21 | 5.70122301 | 7.82568809 | 6.38571982 | 6.05359643 |
| Tnfsf10 | 4.37639922 | 6.48140769 | 0 | 0 |
| Tnfsf12 | 0 | 0 | 0 | 0 |
| Tob1 | 9.18275412 | 0 | 5.06745741 | 5.90038553 |
| vWF | 0 | 0 | 0 | 0 |
| Zbtb20 | 10.0142217 | 8.86759709 | 9.14684532 | 6.41102139 |
| Zbtb38 | 8.26590238 | 9.71780996 | 10.6136333 | 8.51332267 |
| Zfp532 | 3.78349621 | 0 | 4.01404165 | 4.28805397 |
| Zfp612 | 6.67634499 | 0 | 0 | 0 |
| Zfpm1 | 0 | 6.52079531 | 0 | 0 |
| Zhx2 | 0 | 8.45764455 | 7.05698459 | 0 |

TABLE 7-1
Single cell expression data (reduced list)—iHSC-8-TF

| | iHSC-8- | iHSC-8- | iHSC-8- | iHSC-8- | iHSC-8- | iHSC-8- |
|---|---|---|---|---|---|---|
| Factor | TF1 | TF2 | TF3 | TF4 | TF5 | TF6 |
| Actb | 15.3406135 | 15.3198955 | 12.6214841 | 13.9265913 | 14.907027 | 15.0828458 |
| Aebp2 | 5.851253 | 6.91015329 | 6.18045816 | 6.13677942 | 6.31619136 | 6.55729075 |
| Ahr | 0 | 0 | 0 | 0 | 0 | 0 |
| Akt1 | 10.3432926 | 10.2118447 | 8.44749976 | 8.43295768 | 11.0465135 | 11.5937761 |
| Akt2 | 3.80481193 | 4.13073296 | 3.84759163 | 4.37730874 | 4.24877633 | 0 |
| Akt3 | 6.26062374 | 5.80767709 | 0 | 6.66877618 | 0 | 7.28666292 |
| APC | 7.75143555 | 0 | 0 | 0 | 6.70926589 | 6.91997434 |
| Bad | 0 | 0 | 0 | 0 | 0 | 0 |
| Bax | 10.0841523 | 8.99852595 | 8.53670881 | 7.1491247 | 9.41403376 | 10.0713208 |
| Bcl11a | 0 | 3.57733258 | 0 | 0 | 0 | 0 |
| Bcl11b | 0 | 0 | 0 | 5.03025421 | 0 | 0 |
| Bc12 | 3.78836066 | 7.35286615 | 6.11642851 | 5.60720562 | 0 | 4.75013415 |
| Bcl2l1 | 6.11017227 | 0 | 0 | 8.25842512 | 8.41053397 | 10.5350727 |
| Bcl2l11 | 7.53158421 | 0 | 0 | 5.97717038 | 6.54979563 | 7.23702656 |
| Bmi1 | 8.99154721 | 8.57213633 | 1.00536134 | 7.1259908 | 7.77630502 | 9.13913696 |
| Brd3 | 9.63555762 | 6.68960269 | 5.68713764 | 7.26905043 | 7.53751543 | 8.54151772 |
| Casp8 | 8.69580853 | 7.82250438 | 7.27391311 | 7.12647247 | 8.13689545 | 8.33966066 |
| Casp9 | 7.50634956 | 7.89665585 | 8.78122572 | 8.22640477 | 0 | 0 |
| Cbx2 | 7.63597293 | 0 | 0 | 2.88451144 | 6.55755634 | 7.70632981 |
| Cbx8 | 0 | 0 | 0 | 0 | 6.58332722 | 1.23705272 |
| Ccnc | 7.07744906 | 7.39096581 | 7.05379006 | 0 | 8.19654082 | 8.46919791 |
| Ccnd1 | 7.17456113 | 0 | 3.67561661 | 9.15556129 | 0 | 0 |
| Ccne2 | 8.84703835 | 6.74398849 | 0 | 0 | 0 | 0 |
| CD34 | 7.76800322 | 10.2510414 | 2.42976374 | 6.94679739 | 7.33591375 | 0 |
| CD41 | 0 | 7.75482846 | 0 | 0 | 8.70769069 | 0 |
| CD48 | 0 | 7.17814996 | 8.01816633 | 0 | 0 | 9.55567614 |
| CD52 | 10.0135314 | 0 | 11.8982735 | 8.81778186 | 7.57773901 | 11.0136116 |
| CD53 | 10.0270236 | 10.1725729 | 10.2462871 | 7.3567463 | 0 | 10.7604721 |
| CD55 | 4.54836488 | 6.25337777 | 0 | 6.26516647 | 4.55684724 | 5.44238382 |
| CD63 | 5.17005936 | 7.47563153 | 3.07832198 | 6.44407765 | 5.26499364 | 5.17350267 |
| CD9 | 0 | 9.46828366 | 8.37563384 | 6.77430086 | 9.39342697 | 0 |
| Cdc42 | 11.4639526 | 11.5821246 | 9.83848584 | 11.2577485 | 10.7756615 | 12.9047404 |
| Cdk1 | 10.9656852 | 10.4158817 | 0 | 2.26172673 | 7.6531999 | 12.2460627 |
| Cdk4 | 8.77324798 | 9.12698531 | 5.45837872 | 7.85877388 | 6.28997376 | 9.83593049 |
| Cdkn2b | 0 | 0.21758523 | 0 | 0 | 0 | 0 |
| Cebpa | 0 | 4.87998831 | 0 | 0 | 0.53841585 | 0 |
| Csf1r | 8.20143195 | 0 | 0 | 3.50945636 | 0 | 0 |
| Ctnnb1 | 8.29419721 | 8.94929575 | 5.66620169 | 7.85504317 | 8.48239691 | 9.80654905 |
| Cycs | 11.9286577 | 10.5773877 | 9.78151272 | 9.24318367 | 10.6036621 | 13.1484729 |
| Dach1 | 0 | 11.8938366 | 0 | 7.85242012 | 0 | 0 |
| Dnmt1 | 12.431398 | 10.6797953 | 5.10859902 | 8.60332571 | 8.90303261 | 11.5573084 |
| Dnmt3a | 9.31238906 | 0.66595298 | 9.50580001 | 9.36857301 | 8.96311662 | 9.41823059 |
| Dnmt3b | 0 | 0 | 6.81942467 | 4.98217548 | 7.49626958 | 7.95317289 |
| Dtx1 | 0 | 0 | 5.32869997 | 0 | 1.98980211 | 0 |
| Dtx4 | 0 | 0 | 8.14939517 | 0 | 2.92777138 | 8.31531242 |
| Ebf1 | 10.1697266 | 0 | 0 | 0 | 0 | 10.6720985 |
| Ep300 | 9.00180094 | 9.44219254 | 8.29306018 | 8.55233656 | 8.84559399 | 7.94463523 |
| Epor | 7.5372094 | 7.39704832 | 8.33400054 | 7.37800353 | 7.68712078 | 7.35168775 |
| Erg | 10.1327499 | 9.75516364 | 0 | 7.70627287 | 8.62033362 | 12.0140747 |
| Esr1 | 8.88296212 | 9.04098261 | 0 | 6.92108807 | 0 | 8.4763699 |
| ETS1 | 9.58515675 | 7.76396965 | 6.09305906 | 0 | 5.02126265 | 10.27795 |
| ETS2 | 0 | 0 | 0 | 8.15364762 | 0 | 0 |
| Etv3 | 0 | 5.70016295 | 0 | 4.23406152 | 2.35483367 | 0 |
| Etv6 | 7.93361831 | 11.1215646 | 0 | 7.27988804 | 7.89445014 | 8.88475474 |
| Ezh2 | 8.77165156 | 7.66705207 | 4.30929244 | 0 | 0 | 9.57003012 |
| Fas | 0 | 0 | 5.64848062 | 0 | 0 | 0 |
| Fcgr2b | 0.30420554 | 0.45440292 | 5.15394181 | 0 | 0 | 6.8494956 |
| Fcgr3 | 0 | 0 | 4.41247907 | 0 | 1.24977442 | 4.25323357 |
| Fli1 | 10.6596619 | 11.3769697 | 9.56699345 | 9.82489406 | 11.1881229 | 9.43156848 |
| Flt3 | 0 | 8.59953308 | 0 | 0 | 0 | 0 |
| Fosl1 | 0 | 7.74223892 | 0 | 0 | 0 | 0 |
| Foxo1 | 10.5153363 | 9.99673903 | 9.6360569 | 8.19670491 | 6.62389626 | 11.7131359 |
| Foxo3 | 6.94925231 | 8.89744564 | 8.17471245 | 8.28738773 | 8.1441656 | 7.11214992 |
| Gapdh | 8.94923539 | 7.63885103 | 6.1114181 | 6.39966913 | 8.06887865 | 9.83613157 |
| Gata1 | 6.93311607 | 1.95105225 | 4.1024026 | 6.71747066 | 9.80051859 | 7.32322012 |
| Gata2 | 0 | 6.84778411 | 0 | 6.48936067 | 7.0603346 | 3.31930144 |
| Gata3 | 0 | 8.07886909 | 0 | 6.09390185 | 6.13467871 | 0 |
| Gfi1 | 0 | 1.65773111 | 0 | 0 | 0 | 6.42475488 |
| Gfi1b | 0 | 0 | 0 | 8.76265343 | 10.5244821 | 0 |
| Hes5 | 6.16742566 | 0 | 0 | 0 | 0 | 0 |
| Hey1 | 0 | 0 | 0 | 0 | 0 | 0 |
| Hlf | 0 | 10.1536689 | 0 | 8.17012499 | 8.27321734 | 0 |
| Id2 | 0 | 0 | 0 | 0 | 4.45263385 | 4.84341023 |
| Ifi203 | 11.7002151 | 11.6173765 | 10.7830968 | 11.2037766 | 8.89825585 | 10.7833025 |
| Ifi205 | 0 | 0 | 0 | 0 | 0 | 0 |
| Ifitm1 | 8.79797577 | 9.30388568 | 0 | 9.61640866 | 9.63355399 | 0 |
| Ikzf1 | 9.06085707 | 9.97570248 | 9.51200603 | 8.72018894 | 8.01748442 | 10.3459707 |
| Ikzf2 | 0 | 9.19579333 | 0 | 8.34323416 | 0 | 0 |
| Il7R | 0 | 0 | 0 | 0 | 0 | 2.82350147 |
| Irf4 | 6.24028863 | 0 | 11.2249245 | 0 | 0 | 0 |
| Irf6 | 3.86697265 | 4.68374949 | 0 | 0 | 2.8135202 | 0 |
| Irf8 | 8.6858537 | 4.6101286 | 8.99498491 | 0 | 4.89636707 | 9.36420702 |
| Kdr | 0 | 0 | 0 | 0 | 0 | 0 |
| Kit | 8.06121617 | 11.9083565 | 7.88520732 | 10.3475565 | 11.5383331 | 7.96739286 |
| Klf1 | 1.36227074 | 7.02627962 | 0 | 0 | 0 | 0 |
| Klf12 | 0 | 3.7799555 | 3.59391415 | 6.64529932 | 0 | 0 |
| Ldb1 | 8.95380125 | 8.26779513 | 6.67202901 | 7.76543805 | 8.512649 | 10.3751947 |
| Lin28a | 5.97666173 | 0 | 0 | 7.2842936 | 4.1303577 | 4.23775192 |
| Lmo2 | 0 | 9.90783707 | 4.1601552 | 8.76750141 | 9.49745795 | 6.40470448 |
| Ly6a | 6.49157656 | 9.20829801 | 11.7720222 | 8.78675489 | 6.61460984 | 8.7967369 |
| Lyl1 | 3.47100366 | 8.3783465 | 0 | 0 | 0 | 0 |
| Mbd2 | 10.1353897 | 9.91842346 | 7.76162024 | 8.01621694 | 8.98629969 | 11.7384075 |
| Meis1 | 0 | 7.58467677 | 4.18043129 | 6.15361674 | 7.3922156 | 0 |
| Mllt3 | 0 | 0 | 0 | 0 | 0 | 0 |
| Mpl | 0 | 7.78365781 | 0 | 7.84750206 | 9.14807149 | 0 |
| Muc13 | 1.28725247 | 10.3687609 | 0 | 8.47827528 | 8.95782857 | 6.65183597 |
| Myb | 11.2938204 | 11.7723867 | 0 | 10.7012638 | 10.0192772 | 12.3107218 |
| Myc | 6.57202892 | 9.18538633 | 0 | 8.83016864 | 9.14318076 | 10.0463899 |
| Mycn | 0 | 7.76977355 | 5.06288392 | 6.8514822 | 10.8400837 | 0 |
| Ndn | 8.3289328 | 7.37671042 | 0 | 5.16705845 | 7.20854243 | 7.11546949 |
| Nfat5 | 9.5189948 | 10.536889 | 9.07919517 | 9.36357896 | 8.84740478 | 8.99109512 |
| Nfia | 7.94744233 | 7.71267144 | 0 | 8.18008257 | 5.13480173 | 8.01727058 |
| Nfkb1 | 4.49309052 | 0 | 0 | 3.48186805 | 0.74786804 | 0 |
| Notch1 | 0 | 7.53698774 | 7.22766077 | 0 | 0 | 0 |
| Pax4 | 0 | 0 | 0 | 0.90906537 | 0 | 0 |
| Pax5 | 10.5019087 | 0 | 0 | 0 | 0 | 10.127363 |
| Pax9 | 0 | 0 | 0 | 0 | 0 | 0 |
| Pbx1 | 0 | 0 | 0 | 0 | 0 | 0 |
| PIk3ca | 8.87496334 | 9.59446253 | 8.38080955 | 7.92496672 | 7.19725366 | 8.34649914 |
| PIk3R2 | 0 | 9.01075671 | 7.65058108 | 0 | 0 | 8.8251932 |
| Plag1 | 6.21437664 | 0 | 0 | 0 | 0 | 0 |
| Prf1 | 5.13052494 | 0 | 0 | 5.10255205 | 1.86255408 | 0 |
| Pten | 10.4209011 | 9.40062124 | 8.96322075 | 9.10909358 | 9.71271677 | 11.3745533 |
| Rb1 | 11.498329 | 7.96524059 | 9.94840657 | 8.51800071 | 8.72633492 | 10.2612969 |
| Rora | 4.6565537 | 4.45455454 | 4.29766187 | 0 | 6.78445169 | 0 |
| Runx1 | 0 | 3.59548673 | 0 | 0 | 8.70903268 | 8.69444499 |
| Runx2 | 0 | 4.8737639 | 0 | 2.43317885 | 2.69308191 | 0 |
| Satb1 | 9.58445099 | 0 | 0 | 0 | 0 | 10.0568223 |
| Sdpr | 0 | 0 | 0 | 3.31280029 | 5.62934476 | 0 |
| Sell | 0 | 9.75709978 | 0 | 6.9298617 | 0 | 8.38589128 |
| Sfpi1 | 7.63770596 | 10.0783626 | 7.41813664 | 9.49550468 | 7.19133526 | 0 |
| Slamf1 | 0 | 0 | 0 | 6.06097964 | 6.25642952 | 0 |
| Smarca4 | 13.0953186 | 10.9600388 | 9.46765173 | 9.90759459 | 9.19212961 | 12.8606875 |
| Sos1 | 5.40387814 | 5.43895529 | 0 | 2.67690483 | 5.14978146 | 4.18611634 |
| Stat1 | 0 | 2.91513401 | 0.07241094 | 0 | 2.60150676 | 0.29458547 |
| Stat3 | 8.81593264 | 10.0143888 | 8.51673559 | 5.70612457 | 9.26273642 | 8.62967589 |
| Stat4 | 7.59462882 | 7.57005869 | 4.91836386 | 6.5553935 | 7.72874787 | 8.74767888 |
| Stat6 | 0 | 9.26322869 | 9.00041636 | 9.18130068 | 9.26639055 | 10.2390779 |
| Suz12 | 10.8674987 | 9.11262594 | 8.17970692 | 0 | 7.7627513 | 10.4085025 |
| Tal1 | 0 | 1.8367319 | 0 | 5.71521273 | 1.96056078 | 0 |
| Tcf3 | 10.5687751 | 0 | 9.21497368 | 0 | 0 | 10.0481927 |
| Tcf4 | 8.34840792 | 10.2104083 | 9.82698659 | 10.0410063 | 8.76568475 | 11.7542786 |
| Tcf7 | 0 | 3.71590064 | 0 | 0 | 0 | 3.04107777 |
| Tek | 0 | 7.63031049 | 0 | 0 | 8.79573534 | 0 |
| Tfrc | 10.7744689 | 9.18072216 | 0 | 6.62621094 | 7.8677122 | 12.4601279 |
| Tgfb1 | 0 | 5.93085307 | 0 | 5.17968196 | 6.39280849 | 0 |
| Tgfb2 | 0 | 0 | 0 | 0 | 0 | 0 |
| Tgfb3 | 0 | 4.2326363 | 0 | 0 | 0 | 0 |
| Tnfrsf1a | 0 | 10.0793196 | 7.131272 | 8.00451161 | 8.93391961 | 6.97464589 |
| Tnfrsf1b | 0 | 7.84101337 | 0 | 6.33601316 | 7.87941437 | 0 |
| Tnfrsf21 | 5.0514495 | 5.18842864 | 0 | 5.90203138 | 3.76922431 | 5.16498449 |
| Tnfsf10 | 5.74118369 | 4.73595896 | 0 | 0 | 0 | 0 |
| Tnfsf12 | 0 | 0 | 0 | 0 | 0 | 0 |
| Tob1 | 0 | 0 | 0 | 0 | 0 | 0 |
| vWF | 0 | 0 | 0 | 0 | 0 | 0 |
| Zbtb20 | 0 | 8.54271536 | 10.2199855 | 6.6588198 | 6.37611928 | 0 |
| Zbtb38 | 6.56462732 | 6.67014526 | 8.73007335 | 7.61868645 | 7.62865123 | 0 |
| Zfp532 | 0 | 0 | 0 | 0 | 0 | 4.60375818 |
| Zfp612 | 0 | 0 | 0 | 0 | 0 | 7.16346579 |
| Zfpm1 | 5.66600566 | 0 | 0 | 0 | 0 | 0 |
| Zhx2 | 0 | 7.63580107 | 9.72406195 | 1.95086519 | 0 | 7.13427169 |

TABLE 7-2
Single cell expression data (reduced list)—iHSC-8-TF

| | iHSC-8- | iHSC-8- | iHSC-8- | iHSC-8- | iHSC-8- | iHSC-8- |
|---|---|---|---|---|---|---|
| Factor | TF7 | TF8 | TF9 | TF10 | TF11 | TF12 |
| Actb | 14.1168122 | 14.2687572 | 15.8641756 | 14.4381106 | 14.3257382 | 14.6272225 |
| Aebp2 | 6.58743305 | 5.66417136 | 5.22379812 | 5.95905614 | 7.01711608 | 6.02218741 |
| Ahr | 0 | 0 | 0 | 0 | 0 | 0 |
| Akt1 | 10.4975255 | 8.19356615 | 10.0511812 | 9.94944796 | 10.0904307 | 9.78983507 |
| Akt2 | 0 | 5.90204274 | 5.55935143 | 0 | 0 | 0 |
| Akt3 | 4.44707058 | 0 | 5.01641454 | 5.89301145 | 6.31601984 | 2.88783769 |
| APC | 0 | 0 | 6.72226741 | 7.01362759 | 0 | 0 |
| Bad | 0 | 0 | 0 | 0 | 0 | 0 |
| Bax | 9.21290548 | 10.3301544 | 9.28539174 | 6.90668957 | 8.43007045 | 7.04487576 |
| Bcl11a | 0 | 0 | 7.05226632 | 6.95413316 | 0 | 0 |
| Bcl11b | 0 | 6.70827939 | 0 | 0 | 2.52118042 | 0 |
| Bcl2 | 0 | 6.16765619 | 5.32242768 | 3.97203709 | 4.00080172 | 6.6941012 |
| Bcl2l1 | 8.66402847 | 0 | 9.00530066 | 8.98651494 | 9.26985486 | 0 |
| Bcl2l11 | 0 | 0 | 3.75189301 | 7.10582142 | 6.50890906 | 7.33360294 |
| Bmi1 | 0 | 6.89348049 | 7.29668045 | 5.02457691 | 7.99433734 | 5.36245978 |
| Brd3 | 7.71511488 | 6.04924659 | 9.23743083 | 7.95074744 | 8.60723746 | 6.91699201 |
| Casp8 | 7.12754238 | 0 | 8.13700313 | 7.47959123 | 7.47348015 | 7.66051539 |
| Casp9 | 9.29896423 | 7.90126543 | 0 | 8.30432388 | 5.33319179 | 6.17992512 |
| Cbx2 | 0 | 6.43063067 | 0 | 6.62363681 | 7.67267315 | 3.20105562 |
| Cbx8 | 0 | 0 | 0 | 0 | 0 | 0 |
| Ccnc | 0 | 6.40890962 | 7.63555762 | 6.20647804 | 0 | 6.24921005 |
| Ccnd1 | 0 | 0 | 0 | 8.82298676 | 3.76604926 | 7.56671747 |
| Ccne2 | 0 | 0 | 1.01221314 | 6.17859245 | 9.00851145 | 0 |
| CD34 | 9.13922982 | 0 | 0 | 10.584104 | 0 | 0 |
| CD41 | 7.61145278 | 0 | 0 | 7.90679374 | 0 | 0 |
| CD48 | 7.21190179 | 8.92159518 | 8.98615136 | 10.0247552 | 6.7930497 | 8.02173906 |
| CD52 | 9.13495653 | 13.2787307 | 10.360447 | 10.8185364 | 10.295746 | 10.2005651 |
| CD53 | 0 | 10.4873969 | 10.0471444 | 10.0930693 | 10.8734016 | 10.6865731 |
| CD55 | 5.57901574 | 4.03055026 | 4.3185467 | 0 | 2.08180138 | 6.99084057 |
| CD63 | 5.12554231 | 0 | 4.77761362 | 7.17726095 | 5.43373369 | 4.70694381 |
| CD9 | 0 | 5.59346873 | 0 | 6.80671919 | 0 | 6.82472844 |
| Cdc42 | 10.5111244 | 11.2158271 | 11.7756836 | 11.2231375 | 11.759242 | 11.3052155 |
| Cdk1 | 0 | 6.83989229 | 11.7557885 | 8.64473028 | 11.1023897 | 7.79953619 |
| Cdk4 | 6.35473944 | 6.496508 | 8.83507662 | 8.83931041 | 9.36294383 | 5.64163688 |
| Cdkn2b | 0 | 0 | 0 | 0 | 0 | 0 |
| Cebpa | 9.04823476 | 0 | 2.69352882 | 9.40045224 | 0 | 0 |
| Csf1r | 0 | 1.30677937 | 6.92694177 | 0 | 0 | 5.59152648 |
| Ctnnb1 | 6.57811297 | 7.88039783 | 9.34406389 | 9.25901152 | 7.83045263 | 7.02810979 |
| Cycs | 9.94917515 | 11.6037181 | 10.8649283 | 11.5428262 | 10.6576893 | 10.8861042 |
| Dach1 | 9.69728721 | 0 | 0 | 8.33375194 | 0 | 0 |
| Dnmt1 | 8.82661641 | 8.89078551 | 11.301271 | 10.5172729 | 11.1892253 | 8.83847491 |
| Dnmt3a | 7.83006841 | 7.52705094 | 9.67344982 | 11.0465423 | 6.51186067 | 9.32316712 |
| Dnmt3b | 0 | 0 | 0 | 8.62689966 | 4.86534252 | 6.40167463 |
| Dtx1 | 0 | 0 | 0 | 0.59861137 | 0 | 0 |
| Dtx4 | 0 | 8.83830171 | 8.41636566 | 0 | 0 | 0 |
| Ebfl | 0 | 0 | 9.22197094 | 0 | 6.9679956 | 0 |
| Ep300 | 8.5523291 | 8.04374842 | 8.98301466 | 10.1771185 | 10.3582779 | 9.66613489 |
| Epor | 6.61213076 | 6.94262834 | 7.09878512 | 8.50640442 | 7.91438576 | 6.14148414 |
| Erg | 8.01216895 | 0 | 10.904728 | 10.0129949 | 10.7400648 | 9.32820082 |
| Esr1 | 0 | 7.15035885 | 7.57306955 | 8.35236198 | 9.77947514 | 0 |
| ETS1 | 0 | 3.60473663 | 11.2257118 | 7.54149304 | 10.6508588 | 8.51473144 |
| ETS2 | 0 | 0 | 0 | 2.44626873 | 0 | 0 |
| Etv3 | 0 | 3.45927904 | 4.8894594 | 4.37811575 | 0 | 0 |
| Etv6 | 9.45530062 | 5.9105115 | 7.89766842 | 9.28460759 | 9.35002095 | 0 |
| Ezh2 | 4.35380876 | 0 | 9.27707588 | 6.82488745 | 9.39229357 | 7.36394339 |
| Fas | 0 | 6.29945666 | 0 | 0 | 0 | 0 |
| Fcgr2b | 0 | 6.38732146 | 0 | 0 | 2.46788098 | 0 |
| Fcgr3 | 0 | 0 | 0 | 0 | 0 | 0 |
| Fli1 | 10.1576383 | 10.8206693 | 11.9187865 | 11.2788817 | 10.703534 | 7.93882312 |
| Flt3 | 0 | 0 | 0 | 0 | 0 | 0 |
| Fosl1 | 0 | 0 | 0 | 7.6654807 | 0 | 0 |
| Foxo1 | 8.18173307 | 7.60923615 | 11.1745002 | 8.28381064 | 10.4449586 | 7.15153855 |
| Foxo3 | 8.0408479 | 5.81741644 | 6.93021419 | 8.73445684 | 6.05213918 | 7.63571997 |
| Gapdh | 6.89740048 | 4.87578711 | 8.93535347 | 8.19662939 | 8.71071609 | 6.0484422 |
| Gata1 | 9.61468987 | 0 | 5.9071982 | 2.11048309 | 5.4250803 | 6.89996511 |
| Gata2 | 6.95268834 | 1.92704567 | 0 | 0 | 0 | 4.03544209 |
| Gata3 | 6.51624104 | 0 | 5.81915409 | 7.20652789 | 0 | 6.15745412 |
| Gfi1 | 0 | 0 | 8.24022584 | 8.33162082 | 8.54743017 | 0 |
| Gfi1b | 0 | 0 | 0 | 0 | 0 | 0 |
| Hes5 | 0 | 0 | 0 | 0 | 0 | 0 |
| Hey1 | 0 | 0 | 0 | 0 | 0 | 0 |
| Hlf | 0 | 0 | 0 | 9.32403067 | 0 | 0 |
| Id2 | 0 | 8.47672002 | 0 | 0 | 0 | 0 |
| Ifi203 | 8.71443714 | 9.91898509 | 11.2916134 | 12.2525286 | 11.6968592 | 10.2006689 |
| Ifi205 | 0 | 0 | 0 | 0 | 0 | 0 |
| Ifitm1 | 8.45511146 | 0 | 3.69283421 | 12.5234623 | 0 | 7.9891841 |
| Ikzf1 | 9.34781477 | 8.16468581 | 10.0890344 | 8.27576881 | 5.59828783 | 9.92149712 |
| Ikzf2 | 6.24292039 | 0 | 0 | 8.36452991 | 0 | 6.57569221 |
| I17R | 0 | 0 | 4.10352619 | 0 | 3.7996169 | 4.54349931 |
| Irf4 | 0 | 0 | 6.02386675 | 0 | 0 | 0 |
| Irf6 | 2.86463898 | 0 | 0 | 2.12737397 | 0 | 1.02930007 |
| Irf8 | 6.58557808 | 8.00355731 | 7.93428618 | 7.19297404 | 3.05664681 | 7.37397995 |
| Kdr | 0 | 0 | 0 | 0 | 0 | 0 |
| Kit | 10.2589067 | 7.85501741 | 7.73411021 | 11.0726033 | 6.84977833 | 3.48477947 |
| Klf1 | 0 | 0 | 0 | 0 | 0 | 0 |
| Klf12 | 0 | 0 | 0 | 0 | 0 | 5.38786195 |
| Ldb1 | 8.48024052 | 7.15652923 | 8.13838568 | 9.97532882 | 8.59067702 | 7.61339925 |
| Lin28a | 4.37557978 | 7.54315374 | 6.6611673 | 8.6930828 | 5.55717398 | 6.52150568 |
| Lmo2 | 8.85722605 | 0 | 3.81100563 | 9.91085057 | 2.29006541 | 0 |
| Ly6a | 7.16972478 | 12.3655436 | 0 | 10.1051955 | 0 | 0 |
| Lyl1 | 6.96600063 | 0 | 7.38272032 | 0 | 7.10726678 | 0 |
| Mbd2 | 7.44236082 | 8.00969676 | 10.7184582 | 7.17557655 | 10.2817993 | 6.91322033 |
| Meis1 | 6.6721765 | 0 | 0 | 7.89126204 | 0 | 0 |
| Mllt3 | 2.76806472 | 3.87482965 | 3.75675909 | 0 | 0 | 0 |
| Mpl | 0 | 3.70133444 | 0 | 7.96516188 | 0 | 0 |
| Muc13 | 9.22434143 | 2.95206595 | 0 | 9.00435575 | 4.86915097 | 6.41388415 |
| Myb | 11.2843335 | 0 | 11.8407814 | 11.6847567 | 10.6838134 | 9.98616175 |
| Myc | 7.93764864 | 0 | 0 | 6.9817147 | 0 | 0 |
| Mycn | 8.50979223 | 0 | 0 | 8.87104756 | 0 | 0 |
| Ndn | 0 | 2.31950644 | 6.46122501 | 0 | 8.52326206 | 0 |
| Nfat5 | 9.67674286 | 9.22514461 | 9.53936508 | 10.725362 | 10.3961199 | 9.48647076 |
| Nfia | 0 | 0 | 0 | 7.88567867 | 0 | 0 |
| Nfkb1 | 0 | 4.11255372 | 3.3186588 | 4.06803019 | 3.53872344 | 0 |
| Notch1 | 0 | 0 | 0 | 7.4117428 | 0 | 0 |
| Pax4 | 0 | 0 | 0 | 0 | 0 | 0 |
| Pax5 | 0 | 0 | 10.6232231 | 0 | 10.2526594 | 9.85425333 |
| Pax9 | 0 | 0 | 0 | 0 | 0 | 0 |
| Pbx1 | 4.77796595 | 0 | 0 | 5.89394817 | 0 | 0 |
| PIk3ca | 7.06910008 | 7.7317113 | 9.10120998 | 7.88352097 | 7.35188556 | 7.24036714 |
| PIk3R2 | 0 | 7.20794908 | 0 | 7.60310033 | 0 | 0 |
| Plag1 | 0 | 0 | 0 | 0 | 0 | 0 |
| Prf1 | 0 | 6.93683892 | 0 | 0 | 0 | 7.53304996 |
| Pten | 9.40099595 | 7.481518 | 10.9944646 | 10.6633747 | 9.16883013 | 10.6424771 |
| Rb1 | 7.82300867 | 10.1428432 | 10.6672492 | 8.80739047 | 10.7566543 | 7.64031183 |
| Rora | 5.26511699 | 4.55919881 | 3.56025341 | 0 | 0 | 0 |
| Runx1 | 9.23271499 | 0 | 3.36917166 | 8.86537555 | 9.39215951 | 7.72407872 |
| Runx2 | 0.81275604 | 0 | 0 | 5.00576119 | 0 | 0 |
| Satb1 | 0 | 0 | 8.24704373 | 0 | 8.23134552 | 9.39921429 |
| Sdpr | 0 | 0 | 0 | 3.18733123 | 0 | 0 |
| Sell | 8.88207346 | 0 | 9.44019649 | 0 | 7.16166271 | 0 |
| Sfpi1 | 7.84155525 | 7.29077483 | 8.68860268 | 7.60080318 | 0 | 7.23304962 |
| Slamf1 | 6.22492877 | 0 | 0 | 0 | 0 | 0 |
| Smarca4 | 8.02041815 | 8.75617101 | 13.1046438 | 11.1511994 | 13.0182874 | 12.8133987 |
| Sos1 | 0 | 0 | 5.69285508 | 5.55998031 | 2.30626703 | 3.42009457 |
| Stat1 | 1.97314588 | 1.73607307 | 0 | 3.86647267 | 2.39385509 | 0 |
| Stat3 | 9.12583565 | 8.80346971 | 8.32489816 | 9.58777265 | 7.04362269 | 8.57493998 |
| Stat4 | 0 | 0 | 7.10054367 | 6.547509 | 9.30748517 | 9.01247436 |
| Stat6 | 7.61361468 | 7.70780272 | 9.10788213 | 10.06886 | 8.46082693 | 7.60385028 |
| Suz12 | 8.66924158 | 7.78426963 | 9.89026714 | 9.23254249 | 10.4123574 | 0 |
| Tal1 | 2.37117102 | 0 | 6.30535461 | 6.58054019 | 0 | 7.16548188 |
| Tcf3 | 0 | 9.80006797 | 10.7038707 | 0 | 10.1748615 | 9.50402931 |
| Tcf4 | 10.6046337 | 10.677077 | 11.3483408 | 2.06873405 | 10.6701323 | 10.3793339 |
| Tcf7 | 0 | 2.81355742 | 0 | 0 | 0 | 0 |
| Tek | 0 | 0 | 0 | 0 | 0 | 0 |
| Tfrc | 7.64318103 | 0 | 8.74634259 | 10.161451 | 9.22544138 | 9.23442125 |
| Tgfb1 | 0 | 0 | 5.68279082 | 0 | 0 | 5.63076967 |
| Tgfb2 | 0 | 6.35582439 | 0 | 0 | 3.41038781 | 0 |
| Tgfb3 | 0 | 0 | 6.50340017 | 6.4796621 | 0 | 3.14159544 |
| Tnfrsf1a | 9.13753474 | 8.30559171 | 6.21261252 | 8.77734771 | 0 | 0 |
| Tnfrsf1b | 6.8202573 | 0 | 0 | 8.45197156 | 7.39481301 | 2.16712637 |
| Tnfrsf21 | 4.9636023 | 0 | 3.88334514 | 7.04685483 | 5.86000083 | 1.87974929 |
| Tnfsf10 | 5.83655197 | 0 | 5.38524996 | 5.6592177 | 0 | 6.87832602 |
| Tnfsf12 | 0 | 0 | 0 | 0 | 0 | 0 |
| Tob1 | 0 | 5.42079899 | 0 | 0 | 5.55304429 | 0 |
| vWF | 0 | 0 | 0 | 0 | 0 | 0 |
| Zbtb20 | 0 | 11.116707 | 0 | 7.47693235 | 7.16188955 | 0 |
| Zbtb38 | 6.05752543 | 7.56440082 | 7.45865121 | 7.69697887 | 7.27478686 | 2.68202784 |
| Zfp532 | 0 | 0 | 0 | 0 | 0 | 0 |
| Zfp612 | 0 | 0 | 0 | 7.66883285 | 0.41563857 | 6.98993492 |
| Zfpm1 | 0 | 0 | 0 | 0 | 0 | 0 |
| Zhx2 | 0 | 7.97860435 | 7.16760698 | 0 | 0 | 2.5744204 |

TABLE 7-3
Single cell expression data (reduced list)---iHSC-8-TF

| | iHSC-8- | iHSC-8- | iHSC-8- | iHSC-8- | iHSC-8- | iHSC-8- |
|---|---|---|---|---|---|---|
| Factor | TF13 | TF14 | TF15 | TF16 | TF17 | TF18 |
| Actb | 15.4534796 | 15.0457213 | 14.7547847 | 15.7050081 | 14.3181958 | 15.2330791 |
| Aebp2 | 6.93704471 | 4.91542799 | 7.05506882 | 6.86348616 | 6.24968398 | 5.62877356 |
| Ahr | 0 | 6.43180668 | 0 | 0 | 0 | 0 |
| Akt1 | 9.16365108 | 9.30008467 | 10.763603 | 10.9936127 | 9.4294317 | 0 |
| Akt2 | 6.73569225 | 0 | 6.4766602 | 0 | 4.80553304 | 0 |
| Akt3 | 6.11863003 | 6.64875353 | 5.17305023 | 0 | 0 | 0 |
| APC | 0 | 0 | 0 | 0 | 0 | 0 |
| Bad | 0 | 0 | 0 | 0 | 0 | 0 |
| Bax | 10.2213052 | 9.11498692 | 0 | 9.44119327 | 7.49341326 | 8.82070706 |
| Bcl11a | 0 | 0 | 6.11771712 | 0 | 0 | 0 |
| Bcl11b | 0 | 0 | 0 | 0 | 0 | 0 |
| Bcl2 | 7.93705313 | 5.59605449 | 6.02070196 | 4.81608191 | 6.50918987 | 6.69771435 |
| Bcl2l1 | 9.7231417 | 8.58128508 | 10.0362848 | 10.2067064 | 0 | 11.066282 |
| Bcl2l11 | 7.37172881 | 7.69830505 | 7.17734172 | 9.34606481 | 0 | 6.72034529 |
| Bmi1 | 7.34695691 | 7.21167775 | 6.05530861 | 8.46478884 | 8.08106641 | 7.75522788 |
| Brd3 | 8.02785515 | 5.00010534 | 8.51144277 | 9.52747453 | 6.80545653 | 0 |
| Casp8 | 8.37321188 | 7.75230575 | 8.13985014 | 8.57969582 | 8.23333205 | 8.00637905 |
| Casp9 | 7.68090941 | 2.32375499 | 0 | 6.320208 | 8.96183861 | 0 |
| Cbx2 | 6.504426 | 1.54049084 | 7.04621731 | 7.72437829 | 2.30499009 | 0.11481278 |
| Cbx8 | 0 | 0 | 0 | 0 | 0 | 0 |
| Ccnc | 0 | 0 | 7.36621375 | 6.70170152 | 6.00327617 | 6.24461652 |
| Ccnd1 | 6.62265505 | 7.4467213 | 7.52700713 | 6.9456186 | 9.35651788 | 0 |
| Ccne2 | 8.90201474 | 0 | 9.03686227 | 8.45653951 | 4.11742928 | 0 |
| CD34 | 8.79163706 | 9.8829815 | 0 | 0 | 7.5325444 | 0.57823047 |
| CD41 | 10.2235313 | 0 | 10.1703794 | 0 | 0 | 0 |
| CD48 | 7.97202788 | 8.9224792 | 8.76089598 | 9.39765892 | 0 | 8.03809749 |
| CD52 | 0 | 9.99964113 | 10.2992003 | 9.8539851 | 8.86491007 | 12.4913694 |
| CD53 | 0 | 10.8351065 | 0 | 10.056315 | 9.14134727 | 10.1859346 |
| CD55 | 5.74807313 | 0 | 6.03533722 | 4.24146883 | 0 | 0 |
| CD63 | 7.74519483 | 7.00827947 | 6.94140733 | 5.72566979 | 7.10413036 | 4.56443151 |
| CD9 | 8.90957851 | 7.49731749 | 8.92034488 | 0 | 8.64862026 | 5.6361667 |
| Cdc42 | 10.9823548 | 11.4094614 | 11.4173435 | 12.0104029 | 10.8763938 | 12.158319 |
| Cdk1 | 10.1932253 | 0 | 10.4913805 | 0 | 0 | 0 |
| Cdk4 | 8.9755164 | 8.35943257 | 8.53085097 | 8.9627628 | 8.36068234 | 5.6036139 |
| Cdkn2b | 0 | 0 | 1.36381366 | 0 | 0 | 0 |
| Cebpa | 5.893276 | 9.70964699 | 8.88909053 | 0 | 8.03529285 | 0 |
| Csf1r | 0 | 0 | 0 | 1.44879467 | 3.67785521 | 0 |
| Ctnnb1 | 7.48981199 | 8.1336946 | 9.20156778 | 8.61320717 | 7.43105241 | 7.17682577 |
| Cycs | 10.8157891 | 10.720996 | 11.7664034 | 12.1637591 | 8.54219495 | 10.5592201 |
| Dach1 | 8.37404548 | 11.5809914 | 10.0147913 | 0 | 0 | 0 |
| Dnmt1 | 12.1405773 | 8.76320326 | 11.4721676 | 10.43018 | 8.16086858 | 8.31332046 |
| Dnmt3a | 8.03355106 | 10.3047393 | 10.4905211 | 7.34945749 | 9.69684484 | 8.09559308 |
| Dnmt3b | 7.76598102 | 7.12399038 | 7.7635638 | 5.62611906 | 0 | 0 |
| Dtx1 | 0 | 0 | 0 | 3.00328203 | 3.57731956 | 0 |
| Dtx4 | 0 | 0 | 3.31637812 | 0 | 2.0047145 | 0 |
| Ebf1 | 0 | 0 | 0 | 7.91519142 | 0 | 0 |
| Ep300 | 8.3902995 | 8.72299654 | 7.98001879 | 8.27110318 | 8.43022421 | 8.55383003 |
| Epor | 3.5885028 | 6.89489217 | 8.24303376 | 7.15203704 | 8.10722751 | 8.67458521 |
| Erg | 8.06240346 | 8.91508586 | 8.37991482 | 10.1830057 | 9.48944314 | 0 |
| Esr1 | 7.72434085 | 8.96175574 | 0 | 0 | 8.66618842 | 0 |
| ETS1 | 0 | 3.98233032 | 6.85509411 | 10.3680088 | 3.46823602 | 10.4743299 |
| ETS2 | 0 | 0 | 0 | 0 | 7.34802596 | 0 |
| Etv3 | 4.37702979 | 3.36062871 | 4.09342768 | 4.95938064 | 5.16369302 | 0 |
| Etv6 | 10.5013427 | 9.98804513 | 10.0093729 | 8.59292994 | 10.687523 | 2.49925909 |
| Ezh2 | 7.69398439 | 5.90756213 | 7.75922202 | 8.50519978 | 5.14578313 | 0 |
| Fas | 0 | 0 | 0 | 0 | 0 | 5.59986323 |
| Fcgr2b | 5.67144377 | 6.33265476 | 0 | 3.2724894 | 6.59136946 | 0 |
| Fcgr3 | 1.78683374 | 0 | 0 | 0 | 0 | 0 |
| Fli1 | 12.6094269 | 10.2126474 | 11.3348213 | 5.58078903 | 10.5075133 | 10.1080811 |
| Flt3 | 0 | 8.58695173 | 6.08980954 | 0 | 8.01264279 | 0 |
| Fosl1 | 0 | 0 | 0 | 0 | 0 | 0 |
| Foxo1 | 9.24339118 | 9.00386695 | 9.38862847 | 11.2318159 | 9.69497736 | 8.21951707 |
| Foxo3 | 8.23726385 | 8.41088091 | 8.38255114 | 5.96599129 | 7.30821346 | 7.64232189 |
| Gapdh | 8.1689278 | 7.39978258 | 9.08120856 | 9.85054865 | 7.02681009 | 7.07904214 |
| Gata1 | 9.82252363 | 2.06392862 | 8.7659977 | 0 | 0 | 1.37647648 |
| Gata2 | 7.82083798 | 0 | 5.11254203 | 0 | 6.83205962 | 3.78221217 |
| Gata3 | 7.8976454 | 7.41211086 | 6.99172072 | 0 | 6.53375566 | 7.34314284 |
| Gfi1 | 0 | 8.55495398 | 0 | 0 | 9.00701704 | 7.3926737 |
| Gfi1b | 9.48743661 | 8.40675043 | 8.43006036 | 0 | 0 | 0 |
| Hes5 | 0 | 0 | 0 | 0 | 0 | 0 |
| Hey1 | 0 | 0 | 0 | 0 | 0 | 0 |
| Hlf | 7.97486047 | 5.83601979 | 7.98213905 | 0 | 10.5637188 | 0 |
| Id2 | 0 | 0 | 7.30919327 | 0 | 0 | 11.4618755 |
| Ifi203 | 8.94723689 | 10.879845 | 10.187699 | 11.3297255 | 11.4247052 | 11.6820609 |
| Ifi205 | 0 | 0 | 4.25940374 | 0 | 0 | 0 |
| Ifitm1 | 9.9243259 | 12.4987853 | 9.54492255 | 0 | 12.3039462 | 0 |
| Ikzf1 | 8.1181871 | 9.10399583 | 8.67459587 | 10.7026464 | 8.28318113 | 8.02649557 |
| Ikzf2 | 7.07471442 | 9.59931945 | 8.68627507 | 0 | 6.74038185 | 7.10072297 |
| Il7R | 0 | 0 | 0 | 3.30157767 | 0 | 0 |
| Irf4 | 0 | 0 | 0 | 6.54858531 | 0 | 0 |
| Irf6 | 4.17514012 | 0 | 1.48026891 | 0 | 0.90882603 | 0 |
| Irf8 | 0 | 7.73738742 | 0 | 7.77087801 | 0 | 8.75705818 |
| Kdr | 0 | 0 | 0 | 0 | 0 | 0 |
| Kit | 10.468969 | 10.2388187 | 11.4153206 | 10.0405697 | 10.0963359 | 10.0173351 |
| Klf1 | 0 | 0 | 0 | 0 | 0 | 0 |
| Klf12 | 2.45882665 | 0 | 0 | 0 | 0.45168356 | 0 |
| Ldb1 | 9.26322757 | 8.316956 | 9.33639067 | 9.61680777 | 9.26471492 | 8.37672695 |
| Lin28a | 3.6669966 | 6.33329084 | 6.0896607 | 6.57376149 | 0 | 7.17664498 |
| Lmo2 | 8.01911171 | 9.39935871 | 8.30261571 | 4.75634564 | 10.0755277 | 4.24162405 |
| Ly6a | 9.3661827 | 9.11566635 | 0 | 5.99007554 | 10.942034 | 11.0081547 |
| Lyl1 | 8.42712124 | 0 | 0 | 7.18926092 | 5.77433467 | 1.48922057 |
| Mbd2 | 9.41810563 | 8.88421488 | 9.50400444 | 9.47109869 | 6.09034145 | 8.48104688 |
| Meis1 | 7.83156174 | 6.93022589 | 8.37207046 | 0 | 5.60921016 | 0 |
| Mllt3 | 2.67198941 | 0 | 2.16275894 | 0 | 0 | 0 |
| Mpl | 9.75089216 | 7.35694857 | 9.44891837 | 0 | 8.66269181 | 0 |
| Muc13 | 9.13568741 | 8.32054225 | 7.72893994 | 0 | 7.20741875 | 0 |
| Myb | 10.2082629 | 11.5128574 | 11.7486723 | 12.2596713 | 11.0720251 | 0 |
| Myc | 7.83057978 | 9.42868399 | 9.41309135 | 0 | 9.98070807 | 4.55264807 |
| Mycn | 8.6004619 | 6.86525583 | 9.28449734 | 0 | 7.96551367 | 0 |
| Ndn | 7.1716298 | 2.63847367 | 0 | 0 | 0 | 2.27428691 |
| Nfat5 | 10.104519 | 9.08807257 | 8.54046796 | 8.4835257 | 9.84582546 | 9.01999895 |
| Nfia | 9.95835509 | 7.45899512 | 7.64708656 | 0 | 9.2845695 | 7.85053506 |
| Nfkb1 | 0 | 4.7667181 | 1.41310845 | 0 | 4.77499935 | 0 |
| Notch1 | 8.1980529 | 0 | 0 | 6.53133175 | 0 | 5.39606879 |
| Pax4 | 0 | 0 | 0 | 0 | 0 | 6.14490349 |
| Pax5 | 0 | 0 | 0 | 10.708211 | 0 | 0 |
| Pax9 | 0 | 0 | 2.19025952 | 0 | 0 | 0 |
| Pbx1 | 5.99597205 | 0 | 0 | 0 | 0 | 0 |
| PIk3ca | 7.88230828 | 7.47696985 | 7.5500399 | 9.55020565 | 6.23869048 | 0 |
| PIk3R2 | 0 | 8.24510008 | 5.82312994 | 8.02116203 | 8.93406942 | 0 |
| Plag1 | 0 | 0 | 4.73744613 | 0 | 0 | 0 |
| Prf1 | 0 | 0 | 0 | 0 | 0 | 8.73382677 |
| Pten | 9.55201959 | 9.3026472 | 9.49894524 | 11.3146776 | 9.82256436 | 9.31971728 |
| Rb1 | 9.83548418 | 7.73051188 | 10.3125708 | 10.4278048 | 7.92474575 | 8.22490833 |
| Rora | 4.4155683 | 6.32539597 | 0 | 0 | 7.55184114 | 7.19241896 |
| Runx1 | 0 | 5.18013526 | 0 | 9.10402185 | 7.60847018 | 0 |
| Runx2 | 0 | 5.23198449 | 4.54870316 | 0 | 4.71944112 | 3.96352253 |
| Satb1 | 0 | 8.30286654 | 5.48340999 | 9.87087431 | 8.3878369 | 8.22536909 |
| Sdpr | 3.37708308 | 0 | 2.17164004 | 0 | 0 | 0 |
| Sell | 0 | 9.64179428 | 7.32622835 | 8.59354275 | 8.72934132 | 0 |
| Sfpi1 | 9.45330676 | 9.91279299 | 8.0266668 | 8.88627935 | 8.00223079 | 0 |
| Slamf1 | 6.39337217 | 0 | 0 | 0 | 0 | 0 |
| Smarca4 | 8.7128158 | 8.97012069 | 10.9947025 | 12.3094648 | 9.47484623 | 10.0915303 |
| Sos1 | 4.95670739 | 0 | 4.42734538 | 4.56448493 | 4.86048311 | 0 |
| Stat1 | 2.59737419 | 0.92027174 | 2.88493807 | 0.05319102 | 3.02184606 | 6.8812924 |
| Stat3 | 8.06315119 | 9.21638478 | 9.06430179 | 0 | 8.13669425 | 8.30721918 |
| Stat4 | 7.76112821 | 8.49004979 | 8.31123322 | 8.314415 | 9.29957534 | 9.64865985 |
| Stat6 | 9.09210898 | 9.16948618 | 9.52175835 | 7.90699543 | 8.80158849 | 6.97978077 |
| Suz12 | 10.3161732 | 7.41216521 | 10.0021849 | 9.25973518 | 7.31107544 | 7.00993321 |
| Tal1 | 6.27023033 | 4.03587018 | 1.91607573 | 4.92820293 | 0 | 6.45443658 |
| Tcf3 | 0 | 9.72331909 | 0 | 9.28003491 | 0 | 0 |
| Tcf4 | 9.95842877 | 8.92609345 | 10.0138544 | 9.69802767 | 9.51627828 | 0 |
| Tcf7 | 0 | 0 | 3.75075381 | 0 | 0 | 6.71655185 |
| Tek | 0 | 0 | 8.51364933 | 0 | 6.50570444 | 0 |
| Tfrc | 9.36631796 | 7.95001878 | 9.55542439 | 10.7476449 | 8.42531067 | 6.36552267 |
| Tgfb1 | 0 | 0 | 0 | 0 | 0 | 0 |
| Tgfb2 | 0 | 0 | 0 | 0 | 0 | 6.33693857 |
| Tgfb3 | 0 | 0 | 0 | 0 | 0 | 4.26158858 |
| Tnfrsf1a | 8.85163318 | 8.0786507 | 8.1845794 | 7.00521923 | 8.5460922 | 8.06973511 |
| Tnfrsf1b | 7.55637493 | 0 | 7.74358799 | 0 | 5.00485983 | 10.174932 |
| Tnfrs21 | 6.24363175 | 5.64683619 | 5.81156194 | 2.03622926 | 5.64009919 | 0 |
| Tnfsf10 | 0 | 0 | 4.63222478 | 2.26191299 | 6.65398125 | 6.60294222 |
| Tnfsf12 | 0 | 0 | 0 | 0 | 0 | 3.81293855 |
| Tob1 | 5.67117711 | 5.63664714 | 0 | 0 | 0 | 0 |
| vWF | 0 | 0 | 0 | 0 | 0 | 0 |
| Zbtb20 | 5.79959989 | 8.65423374 | 6.14739537 | 0 | 8.35748709 | 4.4844404 |
| Zbtb38 | 8.10033265 | 8.58157099 | 6.34336723 | 7.98028306 | 7.8341961 | 0.56648659 |
| Zfp532 | 0 | 0 | 0 | 0 | 0 | 0 |
| Zfp612 | 5.9361768 | 6.93547371 | 7.20224287 | 6.60794851 | 1.35609575 | 2.25216372 |
| Zfpm1 | 0 | 0 | 6.47136166 | 0 | 7.35425474 | 6.44185159 |
| Zhx2 | 0 | 0 | 7.18849248 | 6.69019455 | 8.13323938 | 8.15233325 |

TABLE 7-4
Single cell expression data (reduced list)-iHSC-8-TF

| | | | iHSC-8- | | iHSC-8- |
|---|---|---|---|---|---|
| Factor | iHSC-8-TF19 | iHSC-8-TF20 | TF21 | iHSC-8-TF22 | TF23 |
| Actb | 15.5949722 | 14.7271674 | 14.9192297 | 14.8524722 | 13.742072 |
| Aebp2 | 6.02657711 | 6.46555858 | 0 | 6.77047 | 5.81780576 |
| Ahr | 0 | 0 | 0 | 0 | 0 |
| Akt1 | 11.1358482 | 10.4380466 | 1.18490888 | 10.7142832 | 7.65650276 |
| Akt2 | 3.53699864 | 6.27657983 | 0 | 0 | 4.99455434 |
| Akt3 | 4.67734217 | 6.17450015 | 4.49098184 | 7.31178082 | 1.69186959 |
| APC | 0 | 6.39404584 | 0 | 8.12096298 | 0 |
| Bad | 0 | 7.94551754 | 0 | 0 | 0 |
| Bax | 9.7269672 | 9.53189139 | 9.22347188 | 8.98116411 | 8.92650111 |
| Bcl11a | 2.68677282 | 0 | 7.45791631 | 4.52048937 | 0 |
| Bcl11b | 0 | 0 | 0 | 5.10896278 | 0 |
| Bcl2 | 6.32982374 | 5.73745116 | 6.26778953 | 4.96019175 | 4.06183255 |
| Bcl2l1 | 8.58581684 | 8.25950033 | 0 | 8.61267991 | 8.15193143 |
| Bcl2l11 | 4.23328455 | 0 | 0 | 8.33934299 | 6.38428587 |
| Bmi1 | 8.09215612 | 6.82056434 | 7.88053812 | 9.25859235 | 7.3067493 |
| Brd3 | 6.26049404 | 8.40584215 | 7.39130082 | 8.74987977 | 6.56313183 |
| Casp8 | 8.55881676 | 9.01946362 | 8.89797827 | 7.89925135 | 7.36966954 |
| Casp9 | 5.69785323 | 6.80005229 | 0 | 0 | 0 |
| Cbx2 | 4.25975897 | 4.50344312 | 0 | 7.05085087 | 7.3652097 |
| Cbx8 | 0 | 3.10519482 | 0 | 0 | 0 |
| Ccnc | 5.68144375 | 7.04800476 | 0 | 7.33402583 | 6.47052476 |
| Ccnd1 | 7.32501662 | 0 | 9.14379317 | 7.80790367 | 8.06188774 |
| Ccne2 | 6.81736138 | 6.12179616 | 4.01589047 | 9.60114654 | 7.32828462 |
| CD34 | 9.10085124 | 10.8245974 | 8.030799 | 0 | 9.90933084 |
| CD41 | 9.18976923 | 8.06311742 | 6.29822743 | 0 | 8.58124579 |
| CD48 | 9.66357797 | 8.92273252 | 0 | 8.22498968 | 9.97655942 |
| CD52 | 9.55607491 | 9.32703404 | 0 | 11.1238367 | 10.7706642 |
| CD53 | 7.4753101 | 10.9421001 | 5.62923652 | 10.1462312 | 9.7742959 |
| CD55 | 6.60757077 | 5.77529023 | 3.89364423 | 0 | 0 |
| CD63 | 7.54605205 | 8.20511072 | 7.4468906 | 4.45819758 | 6.43605916 |
| CD9 | 7.39378017 | 8.69852575 | 0 | 0 | 6.48705589 |
| Cdc42 | 11.6616377 | 11.8297355 | 11.3272877 | 12.0401101 | 11.7498088 |
| Cdk1 | 8.93880406 | 11.8672804 | 0 | 10.8047988 | 6.7710175 |
| Cdk4 | 9.02876243 | 9.33369417 | 7.80946054 | 9.48909263 | 8.12248444 |
| Cdkn2b | 0 | 0 | 0 | 0 | 0 |
| Cebpa | 9.51613005 | 7.67127586 | 0 | 0 | 7.89753488 |
| Csf1r | 2.62576601 | 8.12277306 | 0 | 8.02931482 | 6.8366434 |
| Ctnnb1 | 8.87487327 | 8.44525294 | 5.35747893 | 8.09503157 | 8.19325132 |
| Cycs | 12.2270781 | 12.1725171 | 10.077783 | 12.1894717 | 10.1903946 |
| Dach1 | 8.9706084 | 9.54223727 | 7.78677331 | 0 | 11.0376368 |
| Dnmt1 | 11.4798219 | 11.2665469 | 7.84049793 | 11.501541 | 9.05733118 |
| Dnmt3a | 10.0508273 | 9.21718083 | 8.22085764 | 10.709976 | 7.9890194 |
| Dnmt3b | 8.3035469 | 8.51151681 | 0 | 7.95017678 | 7.02438034 |
| Dtx1 | 0 | 0 | 0 | 5.24802196 | 0 |
| Dtx4 | 8.40755097 | 0 | 0 | 0 | 0 |
| Ebf1 | 0 | 0 | 0 | 10.4583774 | 0 |
| Ep300 | 8.46322899 | 8.98779971 | 7.79215349 | 9.02871727 | 8.54713076 |
| Epor | 7.74276166 | 7.5739171 | 7.09149108 | 7.66734299 | 7.34316306 |
| Erg | 9.42302499 | 10.0003759 | 9.88764997 | 11.1477312 | 10.1170711 |
| Esr1 | 6.07339556 | 7.67273366 | 7.53899738 | 8.109266 | 7.19142983 |
| ETS1 | 0 | 5.65065592 | 4.26833184 | 11.1615476 | 5.0938395 |
| ETS2 | 7.99052669 | 9.33508118 | 7.04822799 | 5.98729189 | 7.4612499 |
| Etv3 | 2.614744 | 3.42408061 | 0 | 2.0997937 | 3.65980713 |
| Etv6 | 8.97419088 | 10.9666148 | 8.76350897 | 0 | 8.62985794 |
| Ezh2 | 6.16548689 | 8.22342442 | 4.15641592 | 9.95784542 | 0 |
| Fas | 0 | 0 | 0 | 0 | 0 |
| Fcgr2b | 0 | 6.31986343 | 0 | 2.15906025 | 5.75272607 |
| Fcgr3 | 0 | 6.13773377 | 0 | 0 | 0 |
| Fli1 | 10.3284821 | 11.6513954 | 11.2427712 | 11.0210733 | 9.6653856 |
| Flt3 | 7.14138117 | 9.90006307 | 8.60740057 | 0 | 9.49158073 |
| Fosl1 | 0 | 2.54124545 | 0 | 0 | 0 |
| Foxo1 | 8.68988455 | 10.7307691 | 8.78369362 | 10.3774981 | 7.14678055 |
| Foxo3 | 8.43953086 | 9.53817626 | 7.34342736 | 7.20503849 | 9.38714958 |
| Gapdh | 9.35145628 | 8.81107493 | 7.38188726 | 9.49765691 | 6.77752673 |
| Gata1 | 6.60958193 | 6.00088041 | 0 | 6.68660622 | 0 |
| Gata2 | 5.15938223 | 6.18940099 | 6.94627744 | 1.11995453 | 0 |
| Gata3 | 3.13442163 | 5.1062862 | 0 | 0 | 6.86901394 |
| Gfi1 | 7.080742 | 9.20777369 | 10.2560592 | 0 | 8.52865693 |
| Gfi1b | 0 | 0 | 6.30300041 | 0 | 0 |
| Hes5 | 3.28111377 | 0 | 0 | 0 | 5.84689612 |
| Hey1 | 0 | 0 | 0 | 0 | 0 |
| Hlf | 8.16750889 | 9.01107414 | 9.44716816 | 0 | 6.3367949 |
| Id2 | 0 | 1.59283696 | 0 | 0 | 6.89130613 |
| Ifi203 | 10.5868051 | 11.9050857 | 11.7792822 | 11.263719 | 10.6719015 |
| Ifi205 | 0 | 0 | 0.57526313 | 0 | 0 |
| Ifitm1 | 12.8767036 | 12.1154443 | 12.6189753 | 0 | 12.8492636 |
| Ikzf1 | 8.97220393 | 10.0637995 | 5.78319283 | 10.2519422 | 9.26900298 |
| Ikzf2 | 9.22178598 | 8.10492715 | 8.78007149 | 0 | 7.86617002 |
| Il7R | 0 | 0 | 0 | 4.64255927 | 0 |
| Irf4 | 0 | 0 | 0 | 6.71439284 | 0 |
| Irf6 | 4.29929913 | 4.17383383 | 4.16212746 | 5.11342417 | 2.29092324 |
| Irf8 | 0 | 0 | 0 | 9.04754663 | 0 |
| Kdr | 0 | 0 | 0 | 0 | 0 |
| Kit | 10.8452909 | 11.46819 | 10.6310949 | 8.0844973 | 10.702966 |
| Klf1 | 0 | 0 | 0 | 0 | 0 |
| Klf12 | 0 | 6.98159901 | 0 | 6.91529257 | 0 |
| Ldb1 | 9.6599478 | 9.52797416 | 8.80192696 | 9.71715245 | 9.66305984 |
| Lin28a | 7.69345152 | 5.72024396 | 7.45549962 | 3.50792444 | 6.18530259 |
| Lmo2 | 9.1278825 | 10.7135692 | 9.55065494 | 0 | 9.24867161 |
| Ly6a | 8.52775889 | 9.24169784 | 10.4450327 | 10.1506563 | 9.23981383 |
| Lyl1 | 0 | 9.23687977 | 6.01993559 | 1.85529048 | 0.14133291 |
| Mbd2 | 9.00365197 | 9.32705014 | 7.01222795 | 10.2980675 | 0 |
| Meis1 | 7.35816194 | 7.96386677 | 7.8940322 | 0 | 5.75544333 |
| Mllt3 | 0 | 0 | 1.20748749 | 3.26279787 | 0 |
| Mpl | 7.07285751 | 8.83332562 | 9.28099881 | 0 | 9.71165465 |
| Muc13 | 8.910028 | 10.4501608 | 9.66072897 | 5.93439112 | 9.95951444 |
| Myb | 12.1660716 | 12.3866801 | 10.9652485 | 11.4963858 | 11.9435595 |
| Myc | 10.5226652 | 8.50048408 | 6.80094773 | 0 | 8.29035189 |
| Mycn | 10.2559863 | 7.33715811 | 8.69237062 | 0 | 10.7681053 |
| Ndn | 0 | 6.24114931 | 0 | 0 | 0 |
| Nfat5 | 0 | 9.94482313 | 9.4521204 | 9.25617131 | 9.31963903 |
| Nfia | 0 | 7.75199021 | 8.40775952 | 0 | 7.32732142 |
| Nfkb1 | 5.05715116 | 3.70671963 | 5.59515553 | 0 | 3.68175399 |
| Notch1 | 0 | 0 | 0 | 7.42247038 | 7.51617552 |
| Pax4 | 1.35750393 | 0 | 0 | 0 | 0 |
| Pax5 | 0 | 0 | 0 | 10.7836978 | 0 |
| Pax9 | 0 | 4.82495586 | 0 | 0 | 0 |
| Pbx1 | 0 | 0 | 0 | 2.22267062 | 0 |
| PIk3ca | 7.79947633 | 9.12079212 | 5.33285433 | 8.74513804 | 6.2959762 |
| PIk3R2 | 9.94903409 | 0 | 7.55937679 | 4.93743794 | 8.18553433 |
| Plag1 | 6.97544118 | 0 | 6.53760217 | 0 | 0 |
| Prf1 | 0 | 0 | 0 | 0 | 0 |
| Pten | 10.0437172 | 11.1348822 | 9.70193974 | 10.5813312 | 10.666182 |
| Rb1 | 7.83303543 | 9.28805228 | 8.58914181 | 11.1046418 | 9.02986546 |
| Rora | 5.99045132 | 4.57639061 | 4.8566497 | 0 | 6.30205008 |
| Runx1 | 0 | 0 | 7.72374854 | 9.29351398 | 9.35240374 |
| Runx2 | 3.63268457 | 5.76424475 | 6.13835151 | 0 | 3.93338711 |
| Satb1 | 7.27713223 | 8.13179502 | 0 | 9.00538844 | 9.07324987 |
| Sdpr | 0 | 0 | 0 | 0 | 0 |
| Sell | 7.51974568 | 0 | 0 | 7.51639506 | 8.23065964 |
| Sfpi1 | 10.3537335 | 10.3438079 | 9.35308484 | 7.34210532 | 10.1166949 |
| Slamf1 | 0 | 0 | 0 | 0 | 0 |
| Smarca4 | 10.6216587 | 11.4880312 | 7.91879599 | 12.7228124 | 10.1231921 |
| Sos1 | 4.77662362 | 5.70044036 | 6.6446615 | 5.15475115 | 4.08108678 |
| Stat1 | 3.68097567 | 3.18143788 | 2.33007484 | 0 | 1.70796503 |
| Stat3 | 9.6835973 | 10.9736276 | 9.82324679 | 8.88395426 | 10.1030436 |
| Stat4 | 8.89602699 | 8.66526465 | 8.61546176 | 9.97092626 | 9.15162945 |
| Stat6 | 8.03975516 | 9.68534085 | 7.65197427 | 8.88963802 | 8.72505432 |
| Suz12 | 9.2920734 | 9.9317102 | 5.55351096 | 11.0088039 | 6.92957444 |
| Tal1 | 0 | 0.5321352 | 0 | 0 | 0 |
| Tcf3 | 0 | 0 | 0 | 10.0357268 | 0 |
| Tcf4 | 8.55403838 | 11.1342293 | 9.5053365 | 11.1326587 | 9.23590743 |
| Tcf7 | 0 | 0 | 0 | 2.09395859 | 0 |
| Tek | 0 | 0 | 0 | 0 | 0 |
| Tfrc | 8.92977574 | 10.3347741 | 0 | 11.2959377 | 0 |
| Tgfb1 | 4.99360374 | 0 | 0 | 0 | 0 |
| Tgfb2 | 6.02165975 | 0 | 0 | 0 | 0 |
| Tgfb3 | 0 | 3.95433485 | 6.38152066 | 7.26386529 | 0 |
| Tnfrsf1a | 8.32699141 | 9.38371569 | 8.98684403 | 0 | 9.01007344 |
| Tnfrsf1b | 7.71407597 | 7.24025508 | 0 | 7.84690402 | 7.6973118 |
| Tnfrsf21 | 5.10082829 | 5.74389161 | 5.35986658 | 0 | 5.6231703 |
| Tnfsf10 | 0 | 5.61139944 | 3.95505967 | 0 | 0 |
| Tnfsf12 | 0 | 0 | 0 | 0 | 0 |
| Tob1 | 0 | 0 | 6.45804508 | 0 | 5.48264167 |
| vWF | 0 | 0 | 3.92489179 | 0 | 0 |
| Zbtb20 | 6.89389913 | 7.17608138 | 0 | 6.53831854 | 6.98907536 |
| Zbtb38 | 6.02485068 | 8.58071957 | 7.5954863 | 5.38002324 | 7.47434598 |
| Zfp532 | 5.25185019 | 0 | 0 | 0 | 0 |
| Zfp612 | 6.35234454 | 2.37453437 | 6.42837344 | 3.48387397 | 6.69808578 |
| Zfpm1 | 0 | 0 | 0 | 0 | 0 |
| Zhx2 | 0 | 0 | 0 | 5.58795878 | 5.46898073 |

TABLE 8-1
Single cell expression data (reduced list)---iHSC-8-TF-Poly

| | iHSC-8- | iHSC-8- | iHSC-8- | iHSC-8- | iHSC-8- | iHSC-8- |
|---|---|---|---|---|---|---|
| Factor | TF-Poly1 | TF-Poly2 | TF-Poly3 | TF-Poly4 | TF-Poly5 | TF-Poly6 |
| Actb | 14.4017745 | 14.2732193 | 15.1526286 | 13.8643652 | 13.9815065 | 14.3047991 |
| Aebp2 | 5.95955683 | 6.89726869 | 6.24332431 | 6.30280532 | 6.9095424 | 7.47978946 |
| Ahr | 9.54980521 | 8.51756005 | 7.1706196 | 0 | 0 | 0 |
| Akt1 | 9.2199823 | 10.5771332 | 10.3125839 | 10.115699 | 8.64780047 | 8.65031952 |
| Akt2 | 5.38910968 | 4.02386518 | 4.9461932 | 0 | 5.38465875 | 0 |
| Akt3 | 7.03433438 | 6.15943216 | 7.67195681 | 7.81890549 | 9.32598867 | 7.96268327 |
| APC | 0 | 6.92782146 | 6.85867754 | 0 | 0 | 7.94220629 |
| Bad | 0 | 0 | 0 | 0 | 0 | 0 |
| Bax | 9.05413463 | 10.0987868 | 10.8354331 | 9.74710118 | 7.76338529 | 8.52100861 |
| Bcl11a | 7.41102372 | 0 | 7.15076275 | 8.58322415 | 8.20030062 | 0 |
| Bcl11b | 7.64367926 | 0 | 3.66716509 | 0 | 1.93636742 | 0 |
| Bcl2 | 3.57531389 | 5.85403867 | 0 | 3.16043824 | 5.61646233 | 5.76077245 |
| Bcl2l1 | 9.07993883 | 8.03643261 | 9.87966794 | 0.83585263 | 8.58326585 | 8.40210943 |
| Bcl2l11 | 0 | 0 | 7.06374493 | 8.39612427 | 0 | 8.4773465 |
| Bmi1 | 9.07560792 | 8.22518209 | 8.42569938 | 0 | 9.40851644 | 7.96975432 |
| Brd3 | 6.95890888 | 6.24785555 | 7.56579491 | 6.62403459 | 6.88629365 | 7.85394619 |
| Casp8 | 7.8559411 | 8.60926927 | 8.8582654 | 6.01680512 | 9.44420835 | 8.42884993 |
| Casp9 | 0 | 8.33784339 | 7.33820605 | 8.25717213 | 8.44629053 | 8.27100862 |
| Cbx2 | 5.65213624 | 0 | 0 | 2.18365236 | 7.0766812 | 3.66755176 |
| Cbx8 | 0 | 0 | 0 | 0 | 0 | 0 |
| Ccnc | 7.23528126 | 7.86231075 | 0 | 7.38487279 | 8.84791023 | 0 |
| Ccnd1 | 0 | 9.72602652 | 7.48420059 | 8.30654599 | 11.8053072 | 11.1237592 |
| Ccne2 | 0 | 4.88759578 | 7.32135738 | 6.93922401 | 0 | 6.77972753 |
| CD34 | 0 | 8.05101797 | 3.40774581 | 8.23829804 | 0 | 0 |
| CD41 | 5.41030089 | 9.39327537 | 7.15100623 | 8.76650086 | 7.87007098 | 8.71774229 |
| CD48 | 0 | 0 | 0 | 0 | 0 | 0 |
| CD52 | 0 | 0 | 0 | 0 | 0 | 0 |
| CD53 | 9.89133699 | 0 | 0 | 8.96185069 | 0 | 0 |
| CD55 | 8.79899388 | 7.63015791 | 5.88277643 | 7.59780097 | 7.37088799 | 7.76280542 |
| CD63 | 8.65376387 | 8.79228248 | 9.15870494 | 6.99196008 | 7.38940631 | 9.44747605 |
| CD9 | 8.16707472 | 7.77311627 | 9.13626418 | 7.43428177 | 6.47201397 | 6.79388862 |
| Cdc42 | 10.6693066 | 12.1804797 | 11.8620482 | 10.497805 | 11.8021081 | 11.9762404 |
| Cdk1 | 0 | 8.11620358 | 7.60561917 | 0 | 0 | 2.42017354 |
| Cdk4 | 8.95820807 | 9.15744736 | 11.0338829 | 8.57125161 | 9.69513549 | 10.0356562 |
| Cdkn2b | 0.46087622 | 0 | 0 | 0 | 0 | 0 |
| Cebpa | 0 | 0 | 0 | 0 | 0 | 0 |
| Csf1r | 0 | 0 | 0 | 6.04286637 | 0 | 0 |
| Ctnnb1 | 8.44935695 | 10.0514987 | 0 | 9.05018407 | 7.94648144 | 9.18714944 |
| Cycs | 6.68979802 | 10.8213383 | 10.6404742 | 9.78073283 | 10.3505161 | 9.81337298 |
| Dach1 | 9.47386037 | 8.81206403 | 7.5999307 | 6.57582267 | 6.70986766 | 7.32706794 |
| Dnmt1 | 10.1960231 | 7.65655217 | 8.31004681 | 8.92673119 | 9.2261255 | 9.71151883 |
| Dnmt3a | 4.24750121 | 7.63469215 | 9.34742168 | 10.0524941 | 10.4262419 | 9.47291437 |
| Dnmt3b | 9.14843642 | 7.69961419 | 7.21411913 | 0 | 8.70429266 | 0 |
| Dtx1 | 0 | 0 | 0 | 5.01837469 | 4.02137797 | 0 |
| Dtx4 | 2.30088686 | 7.91425669 | 4.17934489 | 7.92978791 | 0 | 7.80407419 |
| Ebf1 | 0 | 0 | 0 | 0 | 0 | 0 |
| Ep300 | 8.42978448 | 8.16009533 | 8.11371035 | 8.59805316 | 7.6395129 | 8.21791669 |
| Epor | 6.2878854 | 6.64044771 | 6.75920564 | 8.02055392 | 7.93934358 | 6.20584516 |
| Erg | 8.62942227 | 10.521998 | 10.168764 | 9.83912345 | 9.13177011 | 8.6111314 |
| Esr1 | 9.06471078 | 9.18829675 | 6.19515636 | 11.4378777 | 9.44975997 | 10.8199014 |
| ETS1 | 0 | 8.87124698 | 0 | 8.10142716 | 7.23106564 | 6.79930712 |
| ETS2 | 5.11680482 | 8.0568843 | 8.65044922 | 9.01833153 | 8.46467898 | 7.94602145 |
| Etv3 | 0 | 0 | 0 | 0 | 4.82743292 | 0 |
| Etv6 | 9.79251329 | 9.35978258 | 0 | 10.0075324 | 11.5885534 | 10.1921514 |
| Ezh2 | 5.41817556 | 7.64667858 | 6.75543645 | 0 | 6.4159182 | 6.97011891 |
| Fas | 0 | 0 | 0 | 6.6771592 | 0 | 0 |
| Fcgr2b | 0 | 0 | 0 | 0 | 0 | 1.9110038 |
| Fcgr3 | 0 | 0 | 2.86176005 | 0 | 0 | 2.44845107 |
| Fli1 | 10.5336811 | 12.3667862 | 11.7858238 | 12.4608812 | 10.493611 | 12.4773028 |
| Flt3 | 0 | 0 | 0 | 0 | 0 | 0 |
| Fosl1 | 1.44076501 | 0 | 0 | 0 | 3.94928081 | 0 |
| Foxo1 | 8.43424564 | 8.28876873 | 8.26229198 | 9.69686347 | 10.3959606 | 9.55451527 |
| Foxo3 | 7.99528032 | 8.67713907 | 8.99464508 | 8.9062438 | 9.10399053 | 8.60034284 |
| Gapdh | 9.3099242 | 9.15763066 | 10.6029147 | 9.65043692 | 9.00857274 | 9.93076521 |
| Gata1 | 0.63672388 | 0 | 10.3113521 | 7.1250339 | 4.80520903 | 8.55590577 |
| Gata2 | 6.28005196 | 7.16819061 | 7.23533947 | 7.77620156 | 8.04600994 | 5.86169735 |
| Gata3 | 10.2558503 | 8.50826002 | 6.98895568 | 9.32797131 | 8.9982892 | 8.78943303 |
| Gfi1 | 8.68722923 | 0 | 3.61351347 | 8.08251783 | 9.37851925 | 7.53665623 |
| Gfi1b | 0 | 7.82299121 | 10.8795811 | 9.55418491 | 0 | 10.141432 |
| Hes5 | 0 | 0 | 0 | 0 | 0 | 0 |
| Hey1 | 0 | 0 | 0 | 0 | 0 | 0 |
| Hlf | 10.7996121 | 7.80302654 | 8.07339235 | 10.3328103 | 10.1642256 | 10.9369893 |
| Id2 | 0 | 7.19510114 | 0 | 0 | 7.34117982 | 6.73327638 |
| Ifi203 | 11.2049311 | 11.7941593 | 10.6750846 | 11.8592034 | 11.3822198 | 11.0667002 |
| Ifi205 | 0 | 0 | 0 | 0 | 5.12094266 | 0 |
| Ifitm1 | 13.6239128 | 12.8211493 | 12.5380217 | 13.5860342 | 12.7310037 | 12.7572775 |
| Ikzf1 | 8.02126587 | 9.04043972 | 7.72357321 | 9.05398182 | 9.50868305 | 7.99342233 |
| Ikzf2 | 0 | 7.54783051 | 6.77079194 | 7.44755496 | 8.52813905 | 8.80116026 |
| Il7R | 0 | 0 | 0 | 0 | 0 | 0 |
| Irf4 | 0 | 0 | 5.95460689 | 0 | 0 | 0 |
| Irf6 | 5.93922618 | 6.29386942 | 6.10533594 | 5.63670862 | 6.21974252 | 6.69067944 |
| Irf8 | 6.34567669 | 6.763163 | 0 | 0 | 0 | 0 |
| Kdr | 0 | 7.90283794 | 6.7399962 | 6.04306679 | 7.693852 | 0 |
| Kit | 11.5010613 | 10.5293391 | 7.24957866 | 12.1134045 | 11.2585393 | 10.0523104 |
| Klf1 | 0 | 0 | 0 | 0 | 5.80555786 | 5.1540702 |
| Klf12 | 7.55975795 | 7.04089627 | 7.935156 | 0 | 8.409808 | 3.6068298 |
| Ldb1 | 10.8094981 | 10.8874078 | 10.0963676 | 10.4803974 | 10.0508605 | 10.1714957 |
| Lin28a | 0 | 2.05211875 | 7.65136108 | 8.44983026 | 4.23628819 | 0 |
| Lmo2 | 11.5045036 | 11.4654604 | 12.7062374 | 11.6099483 | 11.790659 | 11.6996282 |
| Ly6a | 11.0781952 | 10.7918825 | 0 | 9.61026549 | 10.6187689 | 9.77041941 |
| Lyl1 | 6.9228556 | 7.88957298 | 7.41124593 | 7.57483786 | 0 | 0.11188596 |
| Mbd2 | 8.86366453 | 9.83898085 | 11.2188215 | 9.25784881 | 10.0178474 | 10.0634688 |
| Meis1 | 8.59070238 | 10.0819024 | 8.56901622 | 8.96918024 | 9.55460124 | 8.92762134 |
| Mllt3 | 0 | 5.19972913 | 6.98132487 | 4.33487907 | 3.43331896 | 4.72749687 |
| Mpl | 10.6098091 | 10.2976387 | 9.44740225 | 9.50399788 | 10.1164058 | 10.0146934 |
| Muc13 | 8.22110323 | 10.3149031 | 10.5075791 | 10.121513 | 6.38829389 | 9.90926088 |
| Myb | 11.3740645 | 11.6070815 | 0 | 11.252238 | 11.1854878 | 11.6427141 |
| Myc | 7.58773767 | 6.92502957 | 11.0745262 | 0 | 10.1593651 | 7.81411074 |
| Mycn | 12.3961119 | 13.821477 | 11.2941091 | 13.4141112 | 13.2655937 | 13.4922153 |
| Ndn | 7.95802745 | 10.8486792 | 9.89395444 | 9.10341388 | 9.93546083 | 10.1963811 |
| Nfat5 | 10.2193279 | 10.6492324 | 8.61806674 | 10.6812757 | 9.9915593 | 9.75117783 |
| Nfia | 8.65330763 | 7.06341868 | 7.38701122 | 9.59475644 | 9.95424844 | 8.85076252 |
| Nfkb1 | 4.446709 | 0 | 6.6481504 | 0 | 2.89270377 | 3.94764604 |
| Notch1 | 0 | 0 | 8.69218776 | 9.10479408 | 0 | 6.95197356 |
| Pax4 | 0 | 0 | 0 | 1.44235065 | 0 | 0 |
| Pax5 | 0 | 0 | 0 | 0 | 6.66633311 | 0 |
| Pax9 | 0 | 5.03638998 | 3.19142852 | 0 | 0 | 0 |
| Pbx1 | 5.79433853 | 2.40166484 | 0 | 6.25602965 | 0 | 0 |
| PIk3ca | 0 | 8.94646056 | 8.24915927 | 9.68680408 | 8.07553724 | 9.42366483 |
| PIk3R2 | 7.86660372 | 7.73972411 | 7.38377942 | 8.09713775 | 8.00818253 | 8.75992262 |
| Plag1 | 0 | 7.49123813 | 5.82502843 | 7.76160342 | 1.23953556 | 9.47539828 |
| Prf1 | 2.80996555 | 0 | 0 | 1.55094842 | 0 | 0 |
| Pten | 10.4165886 | 9.60432119 | 10.2437146 | 9.90287857 | 10.8245223 | 9.89550714 |
| Rb1 | 9.09620227 | 10.2509564 | 7.03917768 | 10.0166256 | 9.88895181 | 10.011227 |
| Rora | 5.67210945 | 8.16786484 | 8.22163059 | 8.40806013 | 8.20332033 | 4.82153142 |
| Runx1 | 10.0392064 | 9.36216612 | 0 | 10.0169963 | 7.55675639 | 1.95995368 |
| Runx2 | 3.02975474 | 0 | 0 | 4.00168042 | 4.49363883 | 3.39036905 |
| Satb1 | 0 | 0 | 6.72850441 | 0 | 0 | 0 |
| Sdpr | 6.47855527 | 7.37567768 | 5.18752317 | 5.78827462 | 4.5789996 | 7.14989941 |
| Sell | 0 | 0 | 0 | 0 | 0 | 0 |
| Sfpi1 | 7.93492701 | 1.16071284 | 8.97426329 | 9.01058427 | 8.8542142 | 8.64133779 |
| Slamf1 | 7.5910261 | 8.53583734 | 7.18007615 | 8.00938404 | 7.5562505 | 8.6742552 |
| Smarca4 | 9.2280708 | 10.369666 | 8.2235885 | 10.7058201 | 10.261829 | 10.5475105 |
| Sos1 | 2.79113487 | 5.88655824 | 7.60011468 | 6.41704302 | 6.34226658 | 5.65496301 |
| Stat1 | 2.30720619 | 2.35055788 | 6.29759725 | 3.85091293 | 5.28729455 | 2.53753709 |
| Stat3 | 10.5102227 | 11.654284 | 7.98961351 | 9.69221977 | 10.9831963 | 9.46455273 |
| Stat4 | 9.73148085 | 9.19610287 | 8.40332968 | 9.9249724 | 8.15997772 | 9.14000192 |
| Stat6 | 8.08137592 | 8.26948638 | 7.50391096 | 0 | 10.2215169 | 8.55245944 |
| Suz12 | 9.3961376 | 9.96724283 | 7.37908318 | 9.47883474 | 9.42011558 | 8.32573094 |
| Tal1 | 1.72237282 | 0 | 6.69073047 | 3.11164048 | 1.32936699 | 0.00662202 |
| Tcf3 | 8.96333241 | 9.31481932 | 0 | 0 | 9.07224108 | 10.1220054 |
| Tcf4 | 8.80005664 | 9.41908139 | 10.3132992 | 8.69843764 | 8.97235944 | 9.3667886 |
| Tcf7 | 0 | 0 | 2.25026637 | 0 | 3.89585347 | 4.39562419 |
| Tek | 4.43072212 | 0 | 0 | 0 | 0 | 8.57224426 |
| Tfrc | 0 | 8.54731767 | 6.89401888 | 9.74317989 | 5.81615029 | 0 |
| Tgfb1 | 0 | 0 | 0 | 0 | 0 | 0 |
| Tgfb2 | 0 | 0 | 6.42618862 | 0 | 0 | 8.02240011 |
| Tgfb3 | 7.17263032 | 0 | 6.69764691 | 8.16263704 | 7.62575941 | 3.60618469 |
| Tnfrsf1a | 9.12239254 | 9.94871547 | 10.5626763 | 8.3415255 | 8.80960043 | 8.44697988 |
| Tnfrsf1b | 7.57265388 | 2.1044987 | 5.61187541 | 9.91624698 | 7.9098197 | 8.62491508 |
| Tnfrsf21 | 4.87454812 | 3.46004955 | 0 | 4.70959999 | 4.73578778 | 4.96266939 |
| Tnfsf10 | 0 | 6.11608237 | 7.18551286 | 8.23570855 | 7.29990668 | 6.85883769 |
| Tnfsf12 | 0 | 0 | 0 | 0 | 0 | 0 |
| Tob1 | 0 | 0 | 0 | 7.63203105 | 5.15771067 | 0 |
| vWF | 7.28131553 | 7.6135713 | 8.13113957 | 7.42453844 | 8.00520062 | 8.84927559 |
| Zbtb20 | 9.1393088 | 8.47880681 | 7.90821765 | 8.9457529 | 8.12571437 | 10.22509 |
| Zbtb38 | 7.37904176 | 9.35075276 | 7.06713579 | 8.59650634 | 6.5271098 | 7.65089916 |
| Zfp532 | 0 | 0 | 7.67157289 | 0 | 0 | 0 |
| Zfp612 | 3.43885333 | 8.66672996 | 0 | 6.73462534 | 0 | 5.03501087 |
| Zfpm1 | 0 | 0 | 7.24131733 | 0 | 0 | 0 |
| Zhx2 | 1.94879631 | 0 | 7.81335591 | 8.46235816 | 8.2166298 | 0 |

TABLE 8-2
Single cell expression data (reduced list)---iHSC-8-TF-Poly

| | iHSC-8- | iHSC-8- | iHSC-8- | iHSC-8- | iHSC-8- | iHSC-8- |
|---|---|---|---|---|---|---|
| Factor | TF-Poly7 | TF-Poly8 | TF-Poly9 | TF-Poly10 | TF-Poly11 | TF-Poly12 |
| Actb | 14.5566982 | 13.615687 | 13.2557353 | 13.9045548 | 13.625207 | 13.6632976 |
| Aebp2 | 7.46754461 | 6.09082663 | 7.88599221 | 3.70216827 | 6.20483355 | 6.71566468 |
| Ahr | 7.777933 | 8.74434412 | 8.10667368 | 7.49909044 | 7.20337973 | 0 |
| Akt1 | 10.2515898 | 10.0377805 | 10.6829232 | 9.27077113 | 10.266825 | 10.5734114 |
| Akt2 | 5.61051736 | 0 | 5.3893609 | 5.11237848 | 5.46400025 | 5.08512838 |
| Akt3 | 6.93473018 | 6.61452163 | 7.44026837 | 7.77588506 | 7.14760449 | 5.28506516 |
| APC | 8.24864591 | 7.30804883 | 6.70709773 | 0 | 0 | 2.08510464 |
| Bad | 0 | 7.83220622 | 0 | 0 | 0 | 0 |
| Bax | 9.48202132 | 8.9969831 | 10.9826718 | 9.37331185 | 9.48416241 | 8.8896616 |
| Bcl11a | 5.55206094 | 0 | 9.30842622 | 0 | 0 | 8.16251064 |
| Bcl11b | 0 | 0 | 4.04933387 | 0 | 6.56686767 | 0 |
| Bcl2 | 5.48513078 | 5.01756113 | 7.17323639 | 4.60865583 | 6.53959776 | 6.15098683 |
| Bcl2l1 | 8.40580553 | 2.85422793 | 8.83253241 | 9.37360231 | 8.97631666 | 7.51350228 |
| Bcl2l11 | 7.06672118 | 0 | 0 | 7.28322794 | 6.13979045 | 2.83394681 |
| Bmi1 | 10.1062229 | 8.64380505 | 8.99015684 | 7.21992126 | 8.87436353 | 0 |
| Brd3 | 7.25721075 | 0 | 7.0965374 | 0 | 7.48140966 | 7.08332896 |
| Casp8 | 7.10606382 | 7.11213334 | 9.13994663 | 8.261719 | 7.95659871 | 4.65164926 |
| Casp9 | 0 | 8.75571495 | 0 | 1.70805493 | 0 | 2.58327705 |
| Cbx2 | 2.75579197 | 0 | 4.17954883 | 2.44741358 | 4.393594 | 5.87793163 |
| Cbx8 | 0 | 0 | 0 | 0 | 0 | 0 |
| Ccnc | 7.23061803 | 9.11473694 | 7.78622312 | 2.54536069 | 6.92719273 | 6.83659195 |
| Ccnd1 | 10.6653784 | 8.89949686 | 9.37926846 | 9.10837155 | 10.9590543 | 9.95508055 |
| Ccne2 | 0 | 0 | 6.67129745 | 0 | 6.26507974 | 7.44075399 |
| CD34 | 7.84002032 | 6.14401226 | 2.96413812 | 0 | 0 | 7.08263627 |
| CD41 | 0 | 0 | 0 | 6.79226229 | 1.8891056 | 7.90833057 |
| CD48 | 0 | 0 | 0 | 0 | 0 | 0 |
| CD52 | 0 | 0 | 0 | 0 | 0 | 0 |
| CD53 | 10.2116886 | 10.7187208 | 7.08173192 | 0 | 7.86597872 | 9.01398982 |
| CD55 | 6.98771698 | 2.38132592 | 7.08507818 | 7.89992021 | 7.15246355 | 6.12899081 |
| CD63 | 9.35889467 | 8.34609702 | 7.4525258 | 8.40948734 | 8.52745636 | 9.28338595 |
| CD9 | 0 | 0 | 0 | 0 | 0 | 7.73063553 |
| Cdc42 | 11.5785879 | 10.5894656 | 10.8671101 | 11.1168037 | 11.7063764 | 11.8716066 |
| Cdk1 | 0 | 7.59230634 | 4.57373649 | 8.26530963 | 0 | 2.79902594 |
| Cdk4 | 10.4501041 | 9.38183794 | 9.45444547 | 9.17523295 | 8.69628583 | 10.0283801 |
| Cdkn2b | 0 | 0 | 0 | 0 | 0 | 2.20414523 |
| Cebpa | 7.67068515 | 0 | 0 | 3.00431304 | 0 | 0 |
| Csf1r | 0 | 0 | 0 | 0 | 0 | 0 |
| Ctnnb1 | 8.98595118 | 8.61438975 | 8.0072686 | 8.55085327 | 8.3102969 | 8.76868574 |
| Cycs | 10.5867211 | 9.35280265 | 9.4126619 | 8.77371577 | 8.18994032 | 9.55716753 |
| Dach1 | 7.9702221 | 8.18463035 | 10.0236829 | 9.42554937 | 8.13824416 | 10.359611 |
| Dnmt1 | 7.80846616 | 7.40084034 | 8.85990662 | 4.70802589 | 7.27623299 | 9.44760185 |
| Dnmt3a | 8.89119048 | 9.27747566 | 10.2871952 | 9.54112251 | 9.5508204 | 10.4670722 |
| Dnmt3b | 7.3240984 | 7.49715046 | 0 | 7.22284209 | 8.54691735 | 5.83497538 |
| Dtx1 | 0 | 0 | 4.9945392 | 0 | 0 | 0 |
| Dtx4 | 4.11683447 | 0 | 0 | 4.01331301 | 0 | 2.68856134 |
| Ebf1 | 0 | 0 | 0 | 0 | 0 | 0 |
| Ep300 | 8.75880732 | 8.22004845 | 7.22949951 | 6.94738149 | 8.43564543 | 7.87430334 |
| Epor | 0 | 7.59352322 | 7.74083769 | 7.95447845 | 6.07198618 | 8.08667718 |
| Erg | 10.8478089 | 10.1398615 | 9.1558768 | 9.52550271 | 9.47527555 | 9.24391502 |
| Esr1 | 9.72073813 | 7.43499017 | 3.58070546 | 9.16795158 | 11.0351211 | 7.34656788 |
| ETS1 | 8.67731549 | 0 | 0 | 8.84936082 | 5.14149904 | 6.25528985 |
| ETS2 | 0 | 8.91107552 | 7.23512522 | 7.43400303 | 2.1535625 | 8.78478124 |
| Etv3 | 3.30675555 | 5.24405155 | 3.81620636 | 6.12231898 | 4.75328706 | 5.44221188 |
| Etv6 | 10.8163092 | 9.32897355 | 10.6859753 | 11.9313243 | 10.2425034 | 11.6285131 |
| Ezh2 | 0 | 0 | 2.21554199 | 0 | 5.32363089 | 0 |
| Fas | 0 | 0 | 0 | 0 | 2.16604599 | 0 |
| Fcgr2b | 3.47793 | 0 | 1.71970146 | 2.23921869 | 0 | 5.04737267 |
| Fcgr3 | 0 | 0 | 0 | 0 | 0 | 2.18806711 |
| Fli1 | 10.8345473 | 11.5409772 | 10.5318652 | 10.662109 | 11.4388002 | 10.1671415 |
| Flt3 | 0 | 0 | 0 | 0 | 0 | 0 |
| Fosl1 | 0 | 0 | 5.8347835 | 0 | 7.70592608 | 7.84850811 |
| Foxo1 | 9.4405956 | 8.63244642 | 9.95832224 | 11.215797 | 9.9252048 | 10.7941741 |
| Foxo3 | 9.38655913 | 8.08321966 | 8.07438022 | 8.8867453 | 7.74085669 | 8.93178924 |
| Gapdh | 8.63027458 | 8.73797671 | 8.14527812 | 9.4866405 | 7.33039136 | 9.67482926 |
| Gata1 | 0 | 0 | 1.55162308 | 0 | 0 | 0 |
| Gata2 | 5.47644994 | 6.97088567 | 8.20284665 | 8.01626434 | 5.91147422 | 6.88724501 |
| Gata3 | 9.08511237 | 9.31182071 | 0 | 9.62706291 | 9.32930381 | 6.65922323 |
| Gfi1 | 0 | 7.22139719 | 6.85380432 | 8.35817389 | 7.00712317 | 0 |
| Gfi1b | 0 | 0 | 7.2469058 | 7.45722502 | 6.87129889 | 6.92216504 |
| Hes5 | 0 | 0 | 0 | 0 | 0 | 0 |
| Hey1 | 0 | 0.67601338 | 0 | 0 | 1.09702737 | 0 |
| Hlf | 10.4853838 | 10.1092492 | 9.53028437 | 9.80884657 | 9.89274135 | 11.105232 |
| Id2 | 7.46080895 | 0 | 0 | 0 | 7.18836307 | 4.4396478 |
| Ifi203 | 11.1510789 | 10.5179013 | 12.3149838 | 11.1576976 | 10.6080303 | 11.3037035 |
| Ifi205 | 0 | 0 | 0 | 5.11004436 | 0 | 0 |
| Ifitm1 | 13.4850079 | 14.3779702 | 11.310825 | 12.3177214 | 13.3652001 | 12.6945896 |
| Ikzf1 | 8.54385455 | 6.97196539 | 8.4861291 | 6.77958196 | 8.19579315 | 6.83946026 |
| Ikzf2 | 8.26817651 | 1.0114979 | 0 | 8.04160023 | 8.17715371 | 7.37397864 |
| Il7R | 0 | 0 | 0 | 0 | 0 | 0 |
| Irf4 | 0 | 0 | 0 | 0 | 0 | 0 |
| Irf6 | 6.51071164 | 5.62197926 | 1.33986609 | 3.6512894 | 5.74729803 | 6.27874544 |
| Irf8 | 7.35064711 | 0 | 5.67817332 | 0 | 0 | 0 |
| Kdr | 0 | 7.43990645 | 0 | 0 | 7.74287744 | 0 |
| Kit | 9.44396168 | 9.99654642 | 9.05604605 | 10.7370375 | 11.1484528 | 9.48452903 |
| Klf1 | 0 | 0 | 0 | 0 | 0 | 2.66895857 |
| Klf12 | 7.83284751 | 1.79551807 | 8.02838739 | 8.41667992 | 7.31689315 | 8.22947494 |
| Ldb1 | 10.8649416 | 11.0311014 | 10.2531103 | 9.96867512 | 9.44479733 | 10.237399 |
| Lin28a | 0.07648021 | 5.44206338 | 2.28808923 | 0 | 4.26911442 | 7.49478468 |
| Lmo2 | 10.3300198 | 11.4044966 | 10.8122837 | 12.0024401 | 10.8122958 | 11.5354295 |
| Ly6a | 11.0261252 | 9.19365169 | 0 | 11.2822375 | 10.9680129 | 10.2245897 |
| Lyl1 | 0 | 0 | 0 | 0 | 8.1627394 | 6.9405754 |
| Mbd2 | 9.778048 | 7.88381457 | 9.85411747 | 8.93004612 | 9.84729194 | 9.50047741 |
| Meis1 | 9.79079972 | 9.26553519 | 9.47724048 | 9.11875429 | 7.83230069 | 9.28003396 |
| Mllt3 | 4.95820732 | 6.82834374 | 3.31729194 | 4.78671361 | 5.72656509 | 5.03058026 |
| Mpl | 10.5885966 | 10.2036925 | 10.3769602 | 9.29493118 | 10.1733655 | 10.194539 |
| Muc13 | 6.47555273 | 4.0744404 | 0.74602045 | 9.11384586 | 9.74461615 | 9.05918759 |
| Myb | 10.7442288 | 9.96147288 | 10.993789 | 10.1482872 | 11.1603183 | 11.6769893 |
| Myc | 7.89827193 | 9.71889144 | 8.37756333 | 6.2345676 | 8.71491271 | 9.57514794 |
| Mycn | 13.0888737 | 11.9671485 | 14.0143762 | 12.1914809 | 11.9099683 | 12.4213923 |
| Ndn | 8.94858448 | 10.4219509 | 7.73679165 | 7.97014772 | 9.18715689 | 9.75918486 |
| Nfat5 | 10.3527976 | 9.84044429 | 9.78500077 | 9.69671217 | 9.49142498 | 10.0570506 |
| Nfia | 8.77963768 | 9.1388192 | 9.92274441 | 7.88222414 | 8.46281343 | 10.5459452 |
| Nfkb1 | 4.42634987 | 0 | 4.92034792 | 4.79418239 | 5.49712885 | 5.77034407 |
| Notch1 | 7.75076794 | 0 | 0 | 0 | 9.00866938 | 7.22412965 |
| Pax4 | 0 | 0 | 0 | 0 | 0 | 0 |
| Pax5 | 0 | 5.51060272 | 0 | 0 | 0 | 0 |
| Pax9 | 0 | 0 | 0 | 0 | 0 | 0 |
| Pbx1 | 0 | 0 | 5.27140189 | 0 | 5.62172032 | 6.67462266 |
| PIk3ca | 9.62050132 | 9.28712078 | 9.2982715 | 8.72600436 | 8.2306778 | 6.59758348 |
| PIk3R2 | 6.02135145 | 0 | 0 | 0 | 0 | 7.90960372 |
| Plag1 | 0 | 6.72260382 | 7.03486336 | 7.18387794 | 4.17261924 | 6.64273979 |
| Prf1 | 0 | 3.90415649 | 0 | 0 | 0.63556078 | 0 |
| Pten | 9.26090346 | 10.2405116 | 10.3794127 | 9.50933483 | 10.4712953 | 8.8938414 |
| Rb1 | 9.66749617 | 7.6292368 | 8.71116734 | 8.9432676 | 4.68235943 | 9.80937685 |
| Rora | 0 | 4.97514677 | 7.9587669 | 7.68976191 | 4.34907105 | 5.02881742 |
| Runx1 | 10.1268518 | 0 | 7.85747808 | 5.75506403 | 9.96928817 | 8.24404878 |
| Runx2 | 5.5286143 | 0 | 3.79093014 | 4.65939933 | 4.88754632 | 0 |
| Satb1 | 0 | 8.4748954 | 0 | 0 | 0 | 0 |
| Sdpr | 5.27902633 | 6.32635852 | 6.5332166 | 0 | 7.17059601 | 4.59848613 |
| Sell | 0 | 0 | 0 | 0 | 0 | 0 |
| Sfpi1 | 9.46010411 | 7.75399359 | 7.72602312 | 9.76515629 | 9.72539923 | 7.02277564 |
| Slamf1 | 8.20190825 | 8.19833438 | 0 | 5.55930467 | 0 | 0 |
| Smarca4 | 9.4413014 | 10.1563545 | 8.79018319 | 8.8549291 | 10.3361654 | 11.228265 |
| Sos1 | 4.54939546 | 6.56343031 | 5.6282784 | 3.49839747 | 6.033343 | 7.34548491 |
| Stat1 | 1.6954329 | 2.46606654 | 4.59411276 | 3.22835285 | 3.56380291 | 2.65186982 |
| Stat3 | 9.7980754 | 9.90644603 | 10.0618227 | 10.0057991 | 9.46974309 | 11.2477057 |
| Stat4 | 10.1144294 | 8.47352328 | 8.70582293 | 8.52494598 | 8.72233963 | 8.2171884 |
| Stat6 | 7.86406378 | 0 | 0 | 8.26236445 | 9.0629236 | 7.69535411 |
| Suz12 | 8.39719356 | 7.93784732 | 8.38043045 | 8.85608556 | 9.42803983 | 9.28167431 |
| Tal1 | 0 | 0.681281 | 0 | 2.08441416 | 0 | 1.70076747 |
| Tcf3 | 0 | 0 | 9.9455106 | 9.29810349 | 9.8282128 | 9.54784562 |
| Tcf4 | 0 | 8.51908255 | 9.24863486 | 10.5880166 | 7.28528289 | 7.66941102 |
| Tcf7 | 4.32833396 | 0 | 0 | 0 | 6.36792384 | 2.47636179 |
| Tek | 7.42071469 | 0 | 7.43721036 | 0 | 0 | 7.67578104 |
| Tfrc | 8.06611575 | 7.71886079 | 8.5698818 | 0 | 0 | 8.80876058 |
| Tgfb1 | 0 | 5.94187127 | 4.16958245 | 1.7066482 | 0 | 7.44368223 |
| Tgfb2 | 0 | 3.64491004 | 0 | 8.61953374 | 0 | 4.82967208 |
| Tgfb3 | 7.96037916 | 2.36951015 | 3.0455015 | 0 | 8.1575853 | 0 |
| Tnfrsf1a | 9.58272277 | 8.66151272 | 9.23558302 | 8.67592568 | 9.37894037 | 9.03022699 |
| Tnfrsf1b | 8.90229636 | 7.93923169 | 5.29156723 | 7.81247487 | 8.26692579 | 8.39371317 |
| Tnfrs21 | 0 | 0 | 0 | 5.44213484 | 4.19136877 | 5.44890931 |
| Tnfsf10 | 0 | 0 | 0 | 5.44208502 | 6.0556815 | 5.34683032 |
| Tnfsf12 | 0 | 0 | 4.3913846 | 0 | 0 | 0 |
| Tob1 | 0 | 0 | 6.16399931 | 0 | 0 | 6.29096864 |
| vWF | 6.35040864 | 6.82666845 | 7.07089703 | 5.30969082 | 6.82119478 | 7.28636659 |
| Zbtb20 | 8.54677311 | 8.62567076 | 8.34955811 | 8.95833222 | 8.46048893 | 10.0348575 |
| Zbtb38 | 8.6859832 | 6.69172463 | 7.38375805 | 6.45223583 | 8.91459553 | 8.06672637 |
| Zfp532 | 0 | 0 | 0 | 0 | 0 | 0 |
| Zfp612 | 8.55308069 | 8.49590308 | 7.30051048 | 0 | 8.54459297 | 8.15113011 |
| Zfpm1 | 0 | 0 | 7.83370461 | 0 | 7.67338465 | 0 |
| Zhx2 | 0 | 0 | 4.36096658 | 9.52701148 | 7.8156659 | 7.69538745 |

TABLE 8-3
Single cell expression data (reduced list)---iHSC-8-TF-Poly

| | iHSC-8- | iHSC-8- | iHSC-8- | iHSC-8- | iHSC-8- | iHSC-8- |
|---|---|---|---|---|---|---|
| Factor | TF-Poly13 | TF-Poly14 | TF-Poly15 | TF-Poly16 | TF-Poly17 | TF-Poly18 |
| Actb | 14.2727767 | 12.7280483 | 14.0956291 | 13.7082256 | 13.0574175 | 13.8899065 |
| Aebp2 | 6.11070016 | 7.67413704 | 5.4199737 | 5.67517041 | 6.12979862 | 6.39309702 |
| Ahr | 0 | 7.60162142 | 8.68953508 | 7.22521443 | 0 | 7.80170326 |
| Akt1 | 10.5537808 | 10.2359843 | 10.1876416 | 10.2045296 | 9.32528266 | 11.2037137 |
| Akt2 | 6.04771205 | 5.46968411 | 4.61114177 | 0.36361906 | 5.15470193 | 6.76905664 |
| Akt3 | 7.46685201 | 8.87527885 | 6.41367312 | 6.57064203 | 7.42714251 | 8.82945036 |
| APC | 5.47404929 | 0 | 0 | 3.30240815 | 0 | 0 |
| Bad | 0 | 0 | 0 | 8.25308495 | 0 | 0 |
| Bax | 9.58600628 | 7.72059484 | 8.90118521 | 9.0595556 | 8.89711711 | 10.2420317 |
| Bcl11a | 0 | 7.2152692 | 0 | 9.99754542 | 8.21413322 | 8.37765853 |
| Bcl11b | 0 | 0 | 0 | 0 | 0 | 5.9803208 |
| Bcl2 | 6.3930411 | 6.07276828 | 6.16216896 | 7.49388797 | 5.68656739 | 0 |
| Bcl2l1 | 8.95652025 | 7.10261013 | 9.81018845 | 5.27192178 | 8.28376117 | 7.94107304 |
| Bcl2l11 | 6.33813274 | 0 | 0 | 5.92621331 | 0 | 0 |
| Bmi1 | 8.66147977 | 8.96414419 | 8.75077682 | 8.37533133 | 8.69114053 | 9.23230416 |
| Brd3 | 8.28803382 | 6.3971659 | 6.25298854 | 7.15381467 | 7.6478676 | 8.17779551 |
| Casp8 | 8.45968253 | 8.1712985 | 7.71775573 | 7.76600997 | 8.57602393 | 7.87394894 |
| Casp9 | 4.45260333 | 0 | 0 | 0 | 4.29714485 | 0 |
| Cbx2 | 2.07247445 | 4.80091864 | 2.61905814 | 0 | 1.54064757 | 4.53169391 |
| Cbx8 | 0 | 0 | 0 | 0 | 0 | 0.67434266 |
| Ccnc | 0 | 8.28176951 | 8.20203458 | 0.20286217 | 7.36331044 | 7.27287576 |
| Ccnd1 | 11.3129135 | 10.4797236 | 8.88976756 | 7.2170424 | 8.33377627 | 9.15479719 |
| Ccne2 | 0 | 0 | 1.50040192 | 0 | 0 | 0 |
| CD34 | 8.22979468 | 0 | 0 | 6.91091458 | 8.44625303 | 7.87973307 |
| CD41 | 0 | 7.16278626 | 0 | 7.18437958 | 0 | 0 |
| CD48 | 0 | 0 | 0 | 0 | 0 | 0 |
| CD52 | 0 | 0 | 0 | 0 | 0 | 0 |
| CD53 | 0 | 8.91427674 | 8.44378297 | 9.13656802 | 0 | 9.74428678 |
| CD55 | 6.01147624 | 5.07787524 | 7.69978384 | 2.8938614 | 7.50395162 | 8.09488889 |
| CD63 | 9.97144686 | 8.71949217 | 8.16499862 | 8.98186831 | 6.4416781 | 9.43079454 |
| CD9 | 9.65832099 | 5.7460499 | 8.59279056 | 7.41372418 | 8.48726798 | 7.98386084 |
| Cdc42 | 12.0879567 | 10.9317607 | 11.4005236 | 11.0823193 | 10.9521574 | 11.5405133 |
| Cdk1 | 0 | 2.72753967 | 0 | 2.05216916 | 0 | 0 |
| Cdk4 | 8.5419578 | 8.78105981 | 9.25298713 | 7.52696871 | 8.30059711 | 9.43641662 |
| Cdkn2b | 0 | 0 | 0 | 0 | 0 | 5.12306489 |
| Cebpa | 0 | 0 | 0 | 0 | 8.64186061 | 0 |
| Csf1r | 0 | 0 | 0 | 0 | 0 | 0 |
| Ctnnb1 | 8.20473117 | 8.50969794 | 8.69357555 | 9.73103801 | 5.608402 | 9.62623328 |
| Cycs | 10.355627 | 8.70346871 | 9.62459322 | 8.44123772 | 8.67759939 | 9.25455509 |
| Dach1 | 9.82088619 | 7.86150494 | 9.96350332 | 8.99831455 | 0 | 10.570503 |
| Dnmt1 | 8.77747907 | 7.53562918 | 0 | 7.44505386 | 8.60952809 | 10.0209151 |
| Dnmt3a | 10.9895968 | 8.80508017 | 9.0263749 | 9.03931586 | 9.52116455 | 9.94330249 |
| Dnmt3b | 9.0938017 | 1.17472267 | 3.10327969 | 0 | 2.84001275 | 8.34532121 |
| Dtx1 | 0 | 0 | 0 | 0 | 0 | 0 |
| Dtx4 | 4.43088049 | 3.87028229 | 4.43041562 | 7.35767066 | 0 | 5.6117422 |
| Ebf1 | 0 | 0 | 0 | 0 | 0 | 0 |
| Ep300 | 9.017599 | 6.78903265 | 7.43151301 | 7.60373336 | 8.45575033 | 7.95781099 |
| Epor | 5.61905305 | 6.57651712 | 6.697122 | 7.72336468 | 7.6721107 | 7.16092395 |
| Erg | 11.2267843 | 11.2338502 | 8.98943025 | 8.67311388 | 10.5300473 | 10.3920801 |
| Esr1 | 9.88779417 | 9.5988785 | 10.7077127 | 9.32817858 | 9.04585226 | 0 |
| ETS1 | 7.00604522 | 8.10866426 | 8.03570905 | 7.99879785 | 4.90118407 | 7.96807866 |
| ETS2 | 9.43655065 | 7.58250039 | 8.78658622 | 7.59607589 | 7.77738844 | 8.52035769 |
| Etv3 | 6.23826064 | 3.83649683 | 5.71839126 | 3.62372678 | 5.97641387 | 4.51702701 |
| Etv6 | 11.5745983 | 9.67915008 | 11.1480528 | 9.02130654 | 10.2698644 | 11.1857554 |
| Ezh2 | 5.31268746 | 0 | 4.20179525 | 6.18588773 | 0 | 6.62582331 |
| Fas | 0 | 0 | 0 | 0 | 1.51519502 | 0 |
| Fcgr2b | 6.48856047 | 4.94876599 | 0 | 0 | 0 | 0 |
| Fcgr3 | 3.61683637 | 0.44366131 | 0 | 0 | 0 | 0 |
| Fli1 | 11.8751419 | 11.3361252 | 12.1903114 | 11.2030884 | 11.240247 | 11.2863366 |
| Flt3 | 0 | 0 | 0 | 0 | 0 | 0 |
| Fosl1 | 9.57090972 | 0 | 7.58226569 | 0 | 0 | 7.82360513 |
| Foxo1 | 10.3871499 | 9.3667248 | 10.4078656 | 9.09496896 | 10.2176456 | 10.0456512 |
| Foxo3 | 8.47876623 | 9.50744661 | 9.2592793 | 7.51365588 | 7.19553746 | 9.10509162 |
| Gapdh | 9.38324817 | 7.33400257 | 8.80742103 | 7.06433381 | 7.70747783 | 9.59697776 |
| Gata1 | 5.31073843 | 0 | 0 | 0 | 1.26264701 | 7.26109145 |
| Gata2 | 6.68669869 | 6.50786707 | 7.6104304 | 3.89707824 | 6.63102054 | 8.2588868 |
| Gata3 | 7.04848734 | 8.94414597 | 8.45487627 | 9.75563278 | 9.27170655 | 10.8195073 |
| Gfi1 | 9.73235707 | 9.86036822 | 8.40070436 | 4.05484467 | 5.30647504 | 8.60826828 |
| Gfi1b | 0 | 0 | 0 | 0 | 0 | 7.65342243 |
| Hes5 | 0 | 0 | 0 | 0 | 0 | 0 |
| Hey1 | 0 | 0 | 0 | 7.33170389 | 0 | 0 |
| Hlf | 9.04765144 | 10.6533675 | 10.5269011 | 9.04230199 | 9.56488914 | 9.70383891 |
| Id2 | 7.62991754 | 0 | 4.23111706 | 0 | 0 | 0 |
| Ifi203 | 11.2501676 | 11.914907 | 11.8653931 | 11.1350751 | 12.3322589 | 11.9786983 |
| Ifi205 | 0 | 0 | 0 | 0 | 0 | 0 |
| Ifitm1 | 13.6024841 | 13.2671579 | 13.1559778 | 14.6147998 | 12.3940005 | 13.0506359 |
| Ikzf1 | 8.99227257 | 0 | 6.59952389 | 4.22155675 | 8.51392841 | 8.28888823 |
| Ikzf2 | 7.97202071 | 9.3328216 | 8.73462182 | 7.32657718 | 7.08686654 | 9.84110991 |
| Il7R | 0 | 0 | 0 | 0 | 0 | 0 |
| Irf4 | 4.70292121 | 0 | 0 | 0 | 0 | 0 |
| Irf6 | 5.54666139 | 6.23688513 | 0 | 5.97785483 | 5.02049373 | 4.96109854 |
| Irf8 | 7.86823205 | 8.15367383 | 2.63621427 | 6.83354507 | 0 | 0 |
| Kdr | 5.53840288 | 0 | 8.15341571 | 7.01205599 | 7.4642774 | 7.1326176 |
| Kit | 11.2607047 | 10.3606009 | 8.79628445 | 11.5915902 | 10.481916 | 11.5872617 |
| Klf1 | 0 | 3.25860663 | 7.84616118 | 0 | 0 | 0 |
| Klf12 | 8.51276514 | 0 | 7.11967734 | 0 | 6.76070903 | 7.21735901 |
| Ldb1 | 10.1909279 | 10.4320215 | 9.54439153 | 10.2617076 | 9.04575239 | 10.805799 |
| Lin28a | 5.62179949 | 3.31473014 | 1.92780466 | 0 | 6.68984894 | 3.16561904 |
| Lmo2 | 11.3839154 | 11.4034046 | 11.3907002 | 10.8211784 | 10.7792744 | 11.5849622 |
| Ly6a | 10.4770569 | 8.56680086 | 10.4469799 | 10.6032693 | 9.78976088 | 10.5262032 |
| Lyl1 | 7.72600868 | 7.4205871 | 7.75834476 | 0 | 2.04943398 | 5.36599153 |
| Mbd2 | 7.18160941 | 0 | 8.37079723 | 8.40944262 | 7.72922325 | 9.44956043 |
| Meis1 | 8.38029564 | 9.48751454 | 8.92807614 | 9.17214844 | 9.24061666 | 9.66150816 |
| Mllt3 | 5.70832826 | 4.43853888 | 6.41671792 | 3.9945214 | 3.62889877 | 5.44463465 |
| Mpl | 9.80451345 | 10.0432958 | 9.24266526 | 8.79519105 | 8.10182066 | 11.1002171 |
| Muc13 | 8.98196707 | 5.59560036 | 5.88707405 | 8.6485199 | 9.85981222 | 8.758467 |
| Myb | 12.229057 | 11.105609 | 10.5930915 | 11.6502743 | 11.2030698 | 12.0392037 |
| Myc | 5.94054515 | 8.26431355 | 0 | 0 | 9.23698786 | 0 |
| Mycn | 12.9133833 | 12.0386919 | 12.9135442 | 11.3734877 | 12.9094945 | 13.2019114 |
| Ndn | 10.1539124 | 10.0168565 | 10.2371109 | 10.6363452 | 9.55015746 | 10.2823756 |
| Nfat5 | 10.0281421 | 9.33420441 | 9.33337438 | 9.24446933 | 9.36691113 | 10.2854003 |
| Nfia | 9.50780688 | 9.55882506 | 9.20366745 | 10.0443654 | 8.63527972 | 8.37856563 |
| Nfkb1 | 4.40547181 | 0 | 6.72539404 | 5.68195326 | 3.69104625 | 2.61534874 |
| Notch1 | 7.92730103 | 0 | 0 | 0 | 1.91842901 | 7.254093 |
| Pax4 | 0 | 0 | 2.75717363 | 0 | 0 | 0 |
| Pax5 | 0 | 0 | 0 | 0 | 0 | 0 |
| Pax9 | 0 | 0 | 0 | 0 | 7.51446706 | 0 |
| Pbx1 | 0 | 0 | 0 | 0 | 7.35355438 | 0 |
| PIk3ca | 9.36193609 | 10.1573699 | 8.69135241 | 7.22797069 | 9.350244 | 9.7945183 |
| PIk3R2 | 9.08458317 | 7.31464789 | 0 | 7.23501761 | 0 | 8.77459895 |
| Plag1 | 9.35742205 | 9.87687278 | 6.76687433 | 9.21256194 | 7.60654426 | 9.96667624 |
| Prf1 | 0 | 0 | 8.2323039 | 0 | 0 | 0 |
| Pten | 10.0306742 | 10.2227214 | 9.46793062 | 8.97227711 | 10.0315494 | 10.1169538 |
| Rb1 | 9.14716883 | 8.05715458 | 9.38141621 | 7.77964535 | 8.78223278 | 9.78773033 |
| Rora | 7.18374293 | 8.46056013 | 5.83820968 | 8.93757151 | 4.90427489 | 0 |
| Runx1 | 10.9790323 | 6.4366202 | 7.3434187 | 9.02591347 | 7.456308 | 8.6424525 |
| Runx2 | 5.06108884 | 4.73894347 | 3.57947524 | 0 | 4.71767067 | 4.01213338 |
| Satb1 | 0 | 0 | 6.988754 | 0 | 0 | 0 |
| Sdpr | 7.25321831 | 1.49255939 | 5.03703907 | 7.36858199 | 5.63016034 | 4.99059297 |
| Sell | 0 | 0 | 0 | 0 | 0 | 0 |
| Sfpi1 | 8.23548593 | 9.30951305 | 8.23896762 | 9.70211776 | 9.06710973 | 9.02501417 |
| Slamf1 | 8.97871652 | 7.69050245 | 8.12971792 | 8.19661263 | 6.49955824 | 9.49238402 |
| Smarca4 | 9.16368267 | 8.86000678 | 7.42507799 | 10.3635361 | 8.42813404 | 9.89775871 |
| Sos1 | 6.95641434 | 6.76871668 | 5.24577661 | 4.77916419 | 5.28495752 | 5.60242229 |
| Stat1 | 2.55560167 | 1.57984978 | 2.47684151 | 3.53475743 | 5.10154814 | 2.60129708 |
| Stat3 | 10.050798 | 10.319077 | 10.6594607 | 10.4305246 | 8.82047476 | 11.132745 |
| Stat4 | 9.33292587 | 6.1179188 | 9.39461735 | 8.20558579 | 8.85019502 | 10.5591988 |
| Stat6 | 9.12089244 | 9.48439599 | 8.23719382 | 8.55868133 | 9.07236102 | 8.98821013 |
| Suz12 | 9.38104801 | 6.98601382 | 9.13046142 | 8.52416999 | 7.65310844 | 8.32511917 |
| Tal1 | 0 | 0 | 3.47169406 | 0 | 2.67263762 | 7.4198786 |
| Tcf3 | 10.5584 | 9.01499115 | 9.3657276 | 0 | 9.47219667 | 9.8412718 |
| Tcf4 | 9.215939 | 9.39183959 | 7.54261135 | 9.26545368 | 9.99166629 | 10.0227825 |
| Tcf7 | 0 | 0 | 0 | 3.59122317 | 0 | 0 |
| Tek | 0 | 0 | 9.1484583 | 3.17123575 | 7.42337143 | 5.89012912 |
| Tfrc | 8.51963706 | 8.20530652 | 8.13700044 | 0 | 8.25042927 | 0 |
| Tgfb1 | 2.78186927 | 6.42869806 | 5.89270974 | 0 | 1.05785152 | 6.22071528 |
| Tgfb2 | 0 | 0 | 0 | 0 | 0 | 0 |
| Tgfb3 | 3.34813 | 0 | 7.79588299 | 7.42980658 | 7.51930126 | 8.10294994 |
| Tnfrsf1a | 8.97610513 | 9.40934119 | 9.51740906 | 8.73586007 | 7.77034164 | 8.68220529 |
| Tnfrsf1b | 7.7581593 | 7.64845624 | 8.38084662 | 0 | 9.10235665 | 9.03749186 |
| Tnfrsf21 | 2.71798644 | 0 | 1.74571738 | 2.68827623 | 3.04822159 | 5.61552431 |
| Tnfsf10 | 6.81258092 | 7.05606832 | 4.88500889 | 6.08129458 | 4.45443159 | 7.17866012 |
| Tnfsf12 | 0 | 0 | 0 | 0 | 3.42201447 | 3.17042749 |
| Tob1 | 6.08956479 | 0 | 1.0254279 | 0 | 4.07499239 | 0 |
| vWF | 8.69641471 | 7.83148927 | 5.7541953 | 7.51750945 | 6.84122876 | 8.68209545 |
| Zbtb20 | 10.7465428 | 8.11584272 | 10.0666657 | 8.72759216 | 8.97604308 | 9.84825138 |
| Zbtb38 | 7.4776121 | 7.71700408 | 8.82991017 | 6.57384818 | 2.72358522 | 8.69891554 |
| Zfp532 | 0 | 0 | 0 | 0 | 0 | 0 |
| Zfp612 | 5.94153564 | 6.7720852 | 7.52351011 | 7.44920631 | 6.39354799 | 7.81847435 |
| Zfpm1 | 7.42741579 | 6.64520623 | 8.37192116 | 0 | 0 | 6.62040347 |
| Zhx2 | 0 | 0 | 2.08517851 | 8.18192171 | 8.6593969 | 7.86530332 |

TABLE 8-4
Single cell expression data (reduced list)---iHSC-8-TF-Poly

| | iHSC-8- | iHSC-8- | iHSC-8- | iHSC-8- | iHSC-8- | iHSC-8- |
|---|---|---|---|---|---|---|
| Factor | TF-Poly19 | TF-Poly20 | TF-Poly21 | TF-Poly22 | TF-Poly23 | TF-Poly24 |
| Actb | 14.0222957 | 14.9852165 | 14.7231936 | 13.0780412 | 13.1822769 | 14.7520851 |
| Aebp2 | 6.09276785 | 5.91339645 | 7.15748106 | 7.15006465 | 6.44734708 | 7.56825651 |
| Ahr | 0 | 7.35656431 | 0 | 0 | 7.39042048 | 0 |
| Akt1 | 10.1537514 | 9.0396397 | 10.8518586 | 10.0130998 | 9.0677075 | 10.2965742 |
| Akt2 | 5.37628872 | 5.62239369 | 4.54633859 | 4.9627968 | 0 | 6.55702093 |
| Akt3 | 6.43567703 | 0 | 8.32809947 | 7.77517295 | 7.00340875 | 6.69568826 |
| APC | 0 | 6.02993274 | 7.1076109 | 0 | 0 | 7.41151949 |
| Bad | 0 | 7.95577502 | 0 | 0 | 0 | 0 |
| Bax | 9.62042258 | 10.1007541 | 9.93762446 | 10.5704358 | 8.58778402 | 9.82062487 |
| Bcl11a | 8.25024263 | 0 | 6.13142565 | 8.06182977 | 0 | 0 |
| Bcl11b | 0 | 0 | 0 | 0 | 0 | 0 |
| Bcl2 | 5.80097299 | 6.96327952 | 5.50955358 | 6.14344881 | 6.33146119 | 8.64834323 |
| Bcl2l1 | 7.19137797 | 9.09460414 | 8.68585536 | 8.37559007 | 7.91961022 | 9.1222599 |
| Bcl2l11 | 7.10099787 | 0 | 0 | 8.66530941 | 7.92945207 | 7.29055975 |
| Bmi1 | 0 | 8.89333432 | 8.82500517 | 8.04845917 | 7.27905634 | 7.66241462 |
| Brd3 | 7.81963512 | 6.53346079 | 8.46718639 | 6.63970649 | 3.58678146 | 8.79527153 |
| Casp8 | 8.16002179 | 7.41674663 | 9.68556501 | 8.98596978 | 7.82524756 | 8.16507587 |
| Casp9 | 8.12191839 | 0 | 0 | 8.20184923 | 6.86433721 | 0 |
| Cbx2 | 5.83990951 | 7.17824899 | 1.13974563 | 0 | 1.69623499 | 5.47697139 |
| Cbx8 | 0 | 0.94186577 | 4.35885212 | 0.62378639 | 0 | 0 |
| Ccnc | 6.44758404 | 7.56469246 | 7.28657546 | 0 | 3.55530815 | 7.0627638 |
| Ccnd1 | 10.2579337 | 10.3894912 | 10.1044493 | 9.85934264 | 7.70190072 | 10.2600958 |
| Ccne2 | 4.05061191 | 7.82199556 | 0 | 0 | 0 | 0 |
| CD34 | 5.35839334 | 1.30106581 | 7.35425184 | 6.61374857 | 6.44471518 | 1.61234414 |
| CD41 | 5.77643219 | 0 | 10.4393533 | 0 | 0 | 10.3495091 |
| CD48 | 0 | 0 | 0 | 0 | 0 | 0 |
| CD52 | 0 | 0 | 0 | 3.01619125 | 0 | 0 |
| CD53 | 8.03999469 | 0 | 0 | 7.50341317 | 10.1028594 | 0 |
| CD55 | 7.33579923 | 5.27016862 | 7.79008222 | 7.56180434 | 6.90429703 | 7.62824401 |
| CD63 | 8.37023042 | 8.80391232 | 9.66493806 | 8.10475976 | 6.51700946 | 8.24520437 |
| CD9 | 7.12446184 | 0 | 7.78614293 | 0 | 8.48314556 | 7.50038252 |
| Cdc42 | 11.193945 | 11.5997344 | 12.2211899 | 11.14451 | 9.02347781 | 11.8346973 |
| Cdk1 | 3.82114993 | 0 | 0 | 0 | 0 | 8.8707332 |
| Cdk4 | 8.72490443 | 9.2366055 | 9.21810563 | 8.92536239 | 7.92269766 | 9.0715251 |
| Cdkn2b | 0 | 0 | 0 | 0 | 0 | 0 |
| Cebpa | 0 | 0.92340397 | 0 | 0 | 1.00115542 | 3.07355052 |
| Csf1r | 0 | 0 | 0 | 0 | 0 | 4.67306388 |
| Ctnnb1 | 9.02013289 | 8.1995723 | 8.88842654 | 7.35118018 | 7.79633098 | 9.06433317 |
| Cycs | 4.40114607 | 10.7395371 | 9.31670975 | 9.50564127 | 8.73967132 | 10.179991 |
| Dach1 | 9.36485262 | 8.82201919 | 0 | 8.05339981 | 0 | 10.8270759 |
| Dnmt1 | 0 | 9.58140407 | 10.0497632 | 8.2793687 | 6.63806785 | 8.17811462 |
| Dnmt3a | 10.9905048 | 9.19877847 | 7.56408268 | 9.58520501 | 8.76598997 | 11.0073815 |
| Dnmt3b | 6.12321822 | 5.91369116 | 6.74621053 | 8.79572673 | 0 | 8.46193889 |
| Dtx1 | 0 | 0 | 4.45860491 | 0 | 4.40787301 | 2.92452083 |
| Dtx4 | 0 | 0 | 0 | 0 | 0 | 4.65526374 |
| Ebf1 | 0 | 0 | 0 | 0 | 0 | 0 |
| Ep300 | 8.31116148 | 9.22743592 | 7.7293946 | 8.80009368 | 7.48345043 | 9.03015668 |
| Epor | 6.59886102 | 8.36411013 | 6.46843364 | 7.45226452 | 6.61055385 | 8.51394952 |
| Erg | 9.78578531 | 7.35912985 | 11.1497111 | 10.1003655 | 10.2588034 | 10.1895405 |
| Esr1 | 10.7256327 | 11.2332794 | 8.62974835 | 10.714868 | 9.12800318 | 7.71830109 |
| ETS1 | 9.50337181 | 9.15865955 | 8.17116294 | 8.01408055 | 7.45017515 | 0 |
| ETS2 | 7.42626021 | 9.43167027 | 7.78315302 | 9.20343927 | 9.3179479 | 8.01612975 |
| Etv3 | 1.3458142 | 5.98695328 | 4.79867027 | 2.51010934 | 4.92346803 | 3.7511546 |
| Etv6 | 10.6179622 | 10.4118422 | 10.2187025 | 9.96156985 | 10.038584 | 9.91374759 |
| Ezh2 | 0 | 7.4963002 | 5.11451697 | 0 | 4.27019431 | 5.00451192 |
| Fas | 0 | 0 | 6.38955508 | 0 | 0 | 6.53357255 |
| Fcgr2b | 0 | 0 | 0 | 0 | 0 | 0 |
| Fcgr3 | 0 | 0 | 0 | 0 | 0 | 4.03293964 |
| Fli1 | 11.3359409 | 10.6665214 | 12.2098328 | 11.3839786 | 12.0700831 | 12.5969288 |
| Flt3 | 0 | 0 | 5.17872234 | 0 | 0 | 0 |
| Fosl1 | 0 | 0 | 0 | 7.88407638 | 0 | 0 |
| Foxo1 | 10.6157657 | 10.0233787 | 10.3312339 | 8.69958676 | 10.0863135 | 8.75473743 |
| Foxo3 | 8.75455393 | 8.2202859 | 9.4323668 | 8.96146302 | 8.37704731 | 9.54868349 |
| Gapdh | 9.39063578 | 9.6332912 | 9.01611712 | 8.48869618 | 7.45420386 | 9.51346889 |
| Gata1 | 2.29550385 | 0 | 2.19508312 | 0 | 0 | 7.81928617 |
| Gata2 | 7.90701459 | 6.57337507 | 7.74249758 | 7.39810444 | 5.78754669 | 7.21810544 |
| Gata3 | 9.25625641 | 9.59194441 | 7.96562707 | 9.02739686 | 9.24201171 | 5.54111636 |
| Gfi1 | 8.16247965 | 9.05106935 | 1.53883386 | 9.20704112 | 8.86848623 | 1.74671367 |
| Gfi1b | 7.48261818 | 0 | 9.01407569 | 7.92225525 | 0 | 10.4544307 |
| Hes5 | 0 | 0 | 0 | 0 | 0 | 0 |
| Hey1 | 0 | 0 | 0.23980869 | 0 | 3.99694016 | 0 |
| Hlf | 10.0172951 | 8.57271376 | 9.52837203 | 9.19521494 | 10.1704945 | 8.00758435 |
| Id2 | 3.69016431 | 6.66309649 | 0 | 8.09079275 | 0 | 0 |
| Ifi203 | 10.7615272 | 11.4665288 | 9.34620527 | 12.029167 | 12.0276813 | 12.3753844 |
| Ifi205 | 0 | 0 | 6.21186981 | 0 | 0 | 0 |
| Ifitm1 | 12.52963 | 14.056977 | 12.1062642 | 14.4446358 | 13.0043214 | 11.5613877 |
| Ikzf1 | 9.49333946 | 8.39564132 | 6.74977708 | 7.60909535 | 7.65040476 | 9.02382942 |
| Ikzf2 | 8.34635213 | 6.9536272 | 8.61475235 | 6.86277574 | 6.23476562 | 7.53972582 |
| Il7R | 0 | 0 | 0 | 0 | 0 | 0 |
| Irf4 | 0 | 0 | 0 | 5.32286189 | 0 | 0 |
| Irf6 | 4.23055125 | 6.62986325 | 5.38490108 | 4.90732154 | 1.6439306 | 3.52949201 |
| Irf8 | 0 | 7.03460532 | 0 | 2.07699694 | 0 | 6.38053878 |
| Kdr | 0 | 0 | 0 | 8.31606549 | 0 | 1.78210879 |
| Kit | 10.3885328 | 6.36619186 | 9.89600505 | 10.6754558 | 10.6599878 | 12.0390472 |
| Klf1 | 0 | 5.96327424 | 0 | 0 | 3.13498357 | 0 |
| Klf12 | 0 | 5.07853345 | 0 | 0 | 5.94120823 | 7.42134808 |
| Ldb1 | 10.1606712 | 9.4851491 | 10.5743575 | 10.1071175 | 9.70318406 | 9.85749521 |
| Lin28a | 7.17967747 | 5.7551298 | 0 | 6.67444585 | 0 | 7.1800316 |
| Lmo2 | 11.3790886 | 10.9990795 | 11.1248884 | 10.5434856 | 10.8867459 | 11.3916155 |
| Ly6a | 9.90063146 | 10.425202 | 9.30350233 | 10.6994618 | 9.83374053 | 7.67564131 |
| Lyl1 | 7.14647222 | 8.82469566 | 7.29100041 | 7.01495401 | 8.09680593 | 0 |
| Mbd2 | 10.0576916 | 9.28619721 | 9.17962218 | 9.39666712 | 0 | 8.99242891 |
| Meis1 | 9.19612035 | 7.20888322 | 9.3457055 | 8.41585689 | 8.18293381 | 9.0030273 |
| Mllt3 | 6.32843166 | 0.74266874 | 4.39498203 | 3.39205835 | 6.23365027 | 3.44437544 |
| Mpl | 8.94809398 | 10.5780332 | 10.4781264 | 10.277185 | 8.87490577 | 9.39242946 |
| Muc13 | 8.27790617 | 0 | 10.4076758 | 6.76806625 | 5.00295934 | 10.3973791 |
| Myb | 11.7453163 | 11.8586016 | 11.7506552 | 11.1696323 | 10.9979778 | 11.7377404 |
| Myc | 9.36837161 | 7.05635853 | 8.52793183 | 9.28427723 | 0 | 11.6017931 |
| Mycn | 13.2729086 | 13.0027169 | 11.5828444 | 12.1822321 | 11.3420486 | 10.4739711 |
| Ndn | 10.1608893 | 0 | 9.76813472 | 10.8699711 | 6.85887785 | 7.86045035 |
| Nfat5 | 9.33700148 | 9.37171439 | 9.61477453 | 10.0347254 | 9.16012698 | 11.0068399 |
| Nfia | 9.19929579 | 8.60111942 | 9.05469309 | 8.45114924 | 7.63071837 | 10.4493102 |
| Nfkb1 | 5.80357529 | 3.19392376 | 5.18644292 | 2.62506816 | 4.67722842 | 4.73163984 |
| Notch1 | 0 | 0 | 7.11863629 | 0 | 0 | 0 |
| Pax4 | 5.85834965 | 0 | 0 | 0 | 0 | 0 |
| Pax5 | 0 | 0 | 0 | 0 | 0 | 0 |
| Pax9 | 1.49067007 | 1.89512232 | 6.48812116 | 0 | 4.05075553 | 0 |
| Pbx1 | 0 | 6.44666705 | 1.43020832 | 5.65796056 | 0 | 5.75884417 |
| PIk3ca | 8.51982982 | 7.20799174 | 6.37633123 | 0 | 0 | 6.46020226 |
| PIk3R2 | 8.38136327 | 8.97464344 | 9.98572262 | 0 | 7.61404741 | 8.48818785 |
| Plag1 | 0 | 0 | 6.4230689 | 7.11287226 | 0 | 0 |
| Prf1 | 0 | 0 | 0 | 0 | 0 | 0 |
| Pten | 10.540168 | 9.73816633 | 10.8896648 | 9.24580983 | 9.87665899 | 10.9693546 |
| Rb1 | 7.31833258 | 9.22662137 | 9.69069735 | 10.0839906 | 8.40316967 | 8.99999716 |
| Rora | 6.73484556 | 0 | 0 | 0 | 9.2605019 | 9.02226435 |
| Runx1 | 0 | 10.6851969 | 8.05120975 | 9.13766939 | 0 | 7.96523554 |
| Runx2 | 4.65669851 | 4.87793717 | 0 | 4.53994772 | 5.95340157 | 5.22261949 |
| Satb1 | 0 | 0 | 0 | 7.42656655 | 7.19081992 | 0 |
| Sdpr | 4.8919743 | 5.24630781 | 5.31896107 | 5.91836204 | 4.45600583 | 6.97989467 |
| Sell | 1.98131911 | 0 | 0 | 0 | 0 | 0 |
| Sfpi1 | 7.85387748 | 7.69052148 | 0 | 0 | 6.83763769 | 0 |
| Slamf1 | 0 | 8.49168885 | 0 | 8.17041428 | 8.03774087 | 8.97604844 |
| Smarca4 | 10.9295084 | 10.7537022 | 10.631709 | 7.01755625 | 0 | 11.2935237 |
| Sos1 | 6.74790018 | 4.84633913 | 6.25614779 | 5.08932828 | 5.99132703 | 7.55749624 |
| Stat1 | 4.09438953 | 3.12874153 | 0.0136088 | 7.49778073 | 3.08878778 | 4.41941405 |
| Stat3 | 10.6461698 | 10.1970393 | 11.6374187 | 10.6737607 | 10.7089761 | 10.407426 |
| Stat4 | 7.94643022 | 7.77936924 | 7.15328942 | 9.69556223 | 8.59968281 | 10.8890815 |
| Stat6 | 10.6283289 | 9.39699663 | 7.46878642 | 10.3247299 | 8.10965668 | 9.61146029 |
| Suz12 | 8.47536799 | 7.00434943 | 7.22403444 | 9.07440769 | 6.61891321 | 9.26075033 |
| Tal1 | 0 | 0.78562075 | 0.32304358 | 0 | 1.05194194 | 3.12802446 |
| Tcf3 | 8.71121837 | 8.47702552 | 9.5832776 | 9.51241599 | 0 | 0 |
| Tcf4 | 8.74989108 | 10.019422 | 9.67827255 | 9.15887745 | 9.0850838 | 11.1528985 |
| Tcf7 | 0 | 1.52995296 | 0 | 0 | 0 | 5.98283478 |
| Tek | 8.29344896 | 0 | 0 | 0 | 7.0599381 | 6.9286127 |
| Tfrc | 8.98222729 | 0 | 8.46857397 | 0 | 0 | 9.30593475 |
| Tgfb1 | 5.18251178 | 0 | 1.45806631 | 6.10276766 | 6.19575758 | 4.43408052 |
| Tgfb2 | 0 | 0 | 0 | 0 | 0 | 6.69513523 |
| Tgfb3 | 4.31080402 | 7.71403034 | 0 | 0 | 8.43808726 | 7.6392649 |
| Tnfrsf1a | 8.37097875 | 9.75633627 | 9.2929424 | 8.88457116 | 9.33603379 | 9.21302132 |
| Tnfrsf1b | 8.69059649 | 8.23787663 | 9.40938599 | 8.83717215 | 8.39065647 | 10.0362648 |
| Tnfrsf21 | 3.20614275 | 6.19102698 | 5.06049798 | 3.05259086 | 3.46771395 | 6.05459577 |
| Tnfsf10 | 0 | 0 | 5.97171916 | 0 | 7.59819331 | 7.31664485 |
| Tnfsf12 | 0 | 0 | 0 | 0 | 0 | 0 |
| Tob1 | 6.69079448 | 6.00223918 | 0 | 3.73540562 | 0 | 5.02457741 |
| vWF | 7.03390478 | 7.00183766 | 6.76991781 | 7.90167655 | 7.3503261 | 8.19082768 |
| Zbtb20 | 8.75751032 | 8.56608423 | 7.87546645 | 9.54728999 | 9.08834794 | 8.98417896 |
| Zbtb38 | 8.42709931 | 6.65368752 | 8.31325825 | 7.64612461 | 5.85086359 | 7.6993122 |
| Zfp532 | 0 | 2.57549982 | 0 | 0 | 0 | 0 |
| Zfp612 | 7.39496006 | 9.86263779 | 8.6174037 | 6.07547603 | 7.44714339 | 7.42549287 |
| Zfpm1 | 0 | 0 | 7.32419209 | 0 | 0 | 7.09081266 |
| Zhx2 | 5.14338261 | 7.9453336 | 7.54993366 | 7.52150615 | 0 | 0 |

TABLE 8-5
Single cell expression data (reduced list)---iHSC-8-TF-Poly

| | iHSC-8- | iHSC-8- | iHSC-8- | iHSC-8- | iHSC-8- | iHSC-8- |
|---|---|---|---|---|---|---|
| Factor | TF-Poly25 | TF-Poly26 | TF-Poly27 | TF-Poly28 | TF-Poly29 | TF-Poly30 |
| Actb | 15.1467264 | 15.0603057 | 14.7898411 | 13.7224541 | 13.1728469 | 12.9889544 |
| Aebp2 | 7.22454633 | 8.054577 | 6.2934136 | 5.40380392 | 6.94511987 | 6.23324236 |
| Ahr | 0 | 0 | 0 | 8.0387708 | 6.82981017 | 0 |
| Akt1 | 10.4393078 | 11.2053361 | 10.3315581 | 9.52591131 | 8.93069445 | 9.8447304 |
| Akt2 | 5.46798025 | 4.36096146 | 0 | 0 | 6.22509388 | 5.58121685 |
| Akt3 | 8.54577868 | 9.10928289 | 6.11061488 | 5.23070804 | 7.20403999 | 7.48254296 |
| APC | 7.54219167 | 8.23602617 | 0 | 3.26916842 | 7.12783167 | 7.33873364 |
| Bad | 0 | 0 | 0 | 0 | 0 | 0 |
| Bax | 9.50825239 | 10.7263374 | 10.1709333 | 8.9480305 | 7.02132481 | 9.08482722 |
| Bcl11a | 0 | 0 | 0 | 0 | 5.41177469 | 5.64855342 |
| Bcl11b | 0 | 0 | 4.08334085 | 0 | 0 | 0 |
| Bcl2 | 3.68995409 | 7.32318474 | 7.06144794 | 6.58939055 | 3.18869428 | 4.94548147 |
| Bcl2l1 | 6.81430281 | 9.83800287 | 9.83067128 | 9.33405878 | 1.18529944 | 0 |
| Bcl2l11 | 9.18689234 | 4.87995875 | 2.32073334 | 7.05754987 | 7.15679605 | 0 |
| Bmi1 | 9.41703263 | 10.590967 | 8.13517912 | 8.21207019 | 7.89416001 | 8.36530966 |
| Brd3 | 7.40062986 | 8.45229557 | 7.37805192 | 6.73549941 | 6.38937753 | 0 |
| Casp8 | 9.06859401 | 9.89552232 | 7.64299925 | 9.08071818 | 6.57464487 | 8.31311348 |
| Casp9 | 3.44991217 | 6.93448309 | 0 | 9.05103431 | 7.48305696 | 8.79567172 |
| Cbx2 | 5.65665485 | 4.81978051 | 5.01321494 | 7.38009168 | 6.31186522 | 7.25681223 |
| Cbx8 | 7.51395854 | 5.26741788 | 0 | 0 | 0 | 0 |
| Ccnc | 3.40126563 | 7.17806544 | 7.78283799 | 8.63152446 | 8.813967 | 6.58765669 |
| Ccnd1 | 10.8599552 | 11.4320536 | 11.3331975 | 7.53991341 | 0 | 9.29046471 |
| Ccne2 | 7.83759047 | 8.65858417 | 0 | 0 | 3.32687121 | 0 |
| CD34 | 6.63187034 | 9.7565564 | 7.40591115 | 8.39371742 | 6.77659879 | 5.99841538 |
| CD41 | 2.14023125 | 8.47542727 | 6.69580828 | 4.98782898 | 0 | 0 |
| CD48 | 0 | 0 | 0 | 0 | 0 | 0 |
| CD52 | 7.91998753 | 8.98451985 | 0 | 4.94138545 | 0 | 5.93717087 |
| CD53 | 6.94204489 | 10.5301752 | 0 | 7.40829181 | 6.96255155 | 9.16158967 |
| CD55 | 2.67695364 | 7.24868997 | 0 | 6.8723678 | 6.65669014 | 0 |
| CD63 | 7.9251335 | 9.70346434 | 8.76574443 | 8.18049221 | 7.4946542 | 8.16601991 |
| CD9 | 5.82915993 | 0 | 7.99497783 | 0 | 0 | 7.22604682 |
| Cdc42 | 12.2364611 | 12.3344896 | 12.0159112 | 11.7559163 | 10.3539974 | 9.97336176 |
| Cdk1 | 0 | 0 | 0 | 6.53350976 | 4.26316228 | 0 |
| Cdk4 | 9.99908798 | 10.1349512 | 9.51946578 | 8.41035443 | 8.08864468 | 8.77958527 |
| Cdkn2b | 0 | 0 | 0 | 0 | 0 | 0 |
| Cebpa | 0 | 0 | 0 | 0 | 2.49157455 | 0 |
| Csf1r | 0 | 0 | 0 | 7.64542858 | 0 | 0 |
| Ctnnb1 | 8.99986283 | 10.3367688 | 8.5457773 | 7.874021 | 8.83840174 | 7.30650645 |
| Cycs | 10.4684479 | 10.2719616 | 11.6179928 | 8.6873144 | 8.85811424 | 9.08493865 |
| Dach1 | 10.2351588 | 10.5375086 | 5.28114978 | 7.94289632 | 9.06367016 | 8.61751831 |
| Dnmt1 | 10.4609244 | 10.9431578 | 9.49692678 | 8.00548457 | 4.47880176 | 8.89034639 |
| Dnmt3a | 10.1900028 | 10.2175853 | 10.5489199 | 10.1873262 | 8.0649379 | 8.61671847 |
| Dnmt3b | 10.4287559 | 7.64484667 | 7.66846002 | 7.47190296 | 7.46314199 | 0 |
| Dtx1 | 2.87373766 | 0 | 4.45487641 | 0 | 3.39237286 | 0 |
| Dtx4 | 8.30188881 | 0 | 3.76809623 | 8.90387752 | 7.33254088 | 0 |
| Ebf1 | 0 | 0 | 0 | 0 | 0 | 0 |
| Ep300 | 9.27663432 | 10.1042304 | 9.68113841 | 8.5374249 | 8.21206612 | 9.24606331 |
| Epor | 7.85270065 | 8.04294538 | 9.16962943 | 8.54759033 | 7.35632339 | 6.14561167 |
| Erg | 8.55265099 | 10.2669084 | 10.2169225 | 7.63186499 | 8.95845922 | 10.2164651 |
| Esr1 | 9.38768526 | 9.97524679 | 0 | 10.8874494 | 7.8990261 | 7.59868432 |
| ETS1 | 6.6308345 | 8.37613488 | 6.37681253 | 7.42772803 | 0 | 0 |
| ETS2 | 8.17680732 | 10.0653554 | 3.38470303 | 8.81529422 | 0 | 7.77351284 |
| Etv3 | 6.11040493 | 0 | 3.98584882 | 3.26053429 | 4.70577394 | 4.48214929 |
| Etv6 | 9.8608361 | 11.5277743 | 11.4810765 | 8.43992379 | 9.20838366 | 11.0463499 |
| Ezh2 | 7.709826 | 6.54832 | 0 | 4.52144944 | 0 | 0 |
| Fas | 0 | 0 | 0 | 0 | 0 | 0 |
| Fcgr2b | 1.90741417 | 0 | 0 | 0 | 4.85415356 | 1.74014502 |
| Fcgr3 | 0 | 0 | 1.08750014 | 0 | 3.68471648 | 0 |
| Fli1 | 11.0534143 | 13.0298511 | 11.2583348 | 10.8538562 | 11.3158563 | 10.8772294 |
| Flt3 | 0 | 4.16157253 | 0 | 0 | 8.03117137 | 0 |
| Fosl1 | 0 | 0 | 0 | 8.2455383 | 0 | 0 |
| Foxo1 | 9.70714029 | 10.6720909 | 10.3788241 | 9.80708641 | 8.26507304 | 10.6496396 |
| Foxo3 | 9.48634989 | 9.86647621 | 7.51118011 | 8.70034889 | 7.37972878 | 8.55743355 |
| Gapdh | 8.86227153 | 8.45555869 | 8.72625477 | 8.41917922 | 8.02370137 | 6.10600952 |
| Gata1 | 0 | 3.80535399 | 0 | 0 | 6.75158933 | 0 |
| Gata2 | 5.91383797 | 8.18298805 | 7.06534352 | 6.42930963 | 4.69341126 | 5.21404746 |
| Gata3 | 9.11573842 | 10.3308833 | 8.31030094 | 9.17077025 | 8.17912775 | 9.3094042 |
| Gfi1 | 0 | 0 | 1.23659601 | 6.90153413 | 8.4360923 | 6.0672508 |
| Gfi1b | 0 | 7.73951118 | 0 | 8.27925976 | 2.60027956 | 0 |
| Hes5 | 0 | 0 | 0 | 0 | 0 | 0 |
| Hey1 | 0 | 4.03507957 | 0 | 0 | 0 | 0 |
| Hlf | 11.6008005 | 10.3681868 | 8.12581134 | 9.33949169 | 9.74960861 | 9.90445603 |
| Id2 | 0 | 0 | 0 | 0 | 7.98559854 | 0 |
| Ifi203 | 13.7479568 | 12.7438712 | 11.8807423 | 11.6897407 | 8.68436391 | 11.1266634 |
| Ifi205 | 0 | 4.09293031 | 0 | 6.42758045 | 0 | 0 |
| Ifitml | 13.859925 | 14.1799111 | 12.4645038 | 13.3616994 | 12.6048996 | 13.2905626 |
| Ikzf1 | 9.27873989 | 10.4587279 | 5.91103149 | 7.22522005 | 7.63638395 | 7.21841248 |
| Ikzf2 | 8.55691698 | 9.00296885 | 0 | 10.0127515 | 7.05646755 | 7.55750237 |
| Il7R | 0 | 0 | 0 | 0 | 0 | 0 |
| Irf4 | 0 | 0 | 0 | 0 | 0 | 3.13466963 |
| Irf6 | 0 | 4.45135084 | 2.0970079 | 4.45935177 | 2.34298554 | 3.11901816 |
| Irf8 | 8.36267886 | 0 | 8.28087448 | 0 | 0 | 0 |
| Kdr | 0 | 0 | 0 | 7.11467704 | 0 | 0 |
| Kit | 7.33440621 | 11.676319 | 12.0482852 | 10.3613984 | 10.8447689 | 9.71837065 |
| Klf1 | 4.6113579 | 0 | 0 | 7.07231232 | 0 | 0 |
| Klf12 | 7.16079482 | 7.39809865 | 7.38280606 | 7.94577018 | 8.65600956 | 7.11655703 |
| Ldb1 | 11.0650833 | 10.7394902 | 9.391079 | 9.69631695 | 9.34063818 | 8.23556142 |
| Lin28a | 8.59487815 | 7.9674739 | 8.97421223 | 4.11702404 | 8.12470644 | 8.71804793 |
| Lmo2 | 10.8175242 | 11.0371363 | 9.96662941 | 10.9024038 | 10.303006 | 9.67048273 |
| Ly6a | 11.3320064 | 10.8896747 | 11.6269362 | 10.7750255 | 8.734268 | 8.94138397 |
| Lyl1 | 0 | 8.45036073 | 8.31542245 | 7.1453941 | 6.78867557 | 0 |
| Mbd2 | 9.82815303 | 7.77519918 | 9.72316715 | 8.71004644 | 0 | 8.71389867 |
| Meis1 | 8.72386921 | 9.27416327 | 7.7021466 | 8.50453784 | 8.4108095 | 7.11187223 |
| Mllt3 | 1.20911588 | 2.90532993 | 3.24157892 | 6.04227027 | 3.56250704 | 3.41569762 |
| Mpl | 8.16713987 | 11.1382076 | 8.84138738 | 9.51523532 | 6.45757591 | 9.14051092 |
| Muc13 | 3.84864206 | 10.6660629 | 10.1548311 | 7.8264378 | 7.56339286 | 8.44043237 |
| Myb | 11.9506659 | 12.679687 | 12.354001 | 11.6763394 | 11.1472311 | 10.8315677 |
| Myc | 0 | 10.0093188 | 8.34807296 | 9.25839322 | 7.84577514 | 7.52780084 |
| Mycn | 10.870635 | 12.9395207 | 12.3151591 | 12.053502 | 12.6255533 | 9.68590773 |
| Ndn | 6.69958267 | 11.2092172 | 8.79795885 | 10.1009021 | 4.07328976 | 8.99463446 |
| Nfat5 | 10.4275502 | 11.0533765 | 9.97984923 | 10.6782945 | 9.95523149 | 10.2518547 |
| Nfia | 8.76693228 | 11.1506945 | 10.3677089 | 9.02919232 | 7.97805043 | 7.23689606 |
| Nfkb1 | 4.92161927 | 7.85783734 | 0 | 5.31107579 | 0 | 5.41888462 |
| Notch1 | 0 | 6.97371909 | 6.50677693 | 8.20930046 | 7.14314591 | 8.77749162 |
| Pax4 | 0 | 0.41579145 | 0 | 0 | 1.78594162 | 0 |
| Pax5 | 0 | 0 | 0 | 0 | 0 | 0 |
| Pax9 | 0 | 1.29709712 | 5.34825344 | 0 | 0 | 0 |
| Pbx1 | 0 | 0 | 4.99498393 | 0 | 4.3948675 | 0 |
| PIk3ca | 7.29512319 | 5.10151123 | 9.26701666 | 8.77108696 | 7.8137764 | 8.06874559 |
| PIk3R2 | 0 | 9.54668408 | 0 | 4.03560663 | 7.63724867 | 8.09289398 |
| Plag1 | 4.05714178 | 7.17110365 | 7.47615183 | 6.78269553 | 6.68706596 | 8.11285307 |
| Prf1 | 0 | 0 | 0 | 0 | 1.76277593 | 0 |
| Pten | 9.67233193 | 10.8750291 | 11.2752335 | 9.07906849 | 9.619202 | 9.54758043 |
| Rb1 | 2.4815274 | 9.83858258 | 9.93875591 | 8.12503051 | 0 | 9.56415776 |
| Rora | 6.2784063 | 7.96217943 | 8.97191919 | 5.69747967 | 6.69619858 | 0 |
| Runx1 | 7.72158429 | 11.5617806 | 8.0209297 | 0 | 7.34188594 | 9.3066077 |
| Runx2 | 6.44168173 | 6.47921853 | 4.05939813 | 0 | 4.52343132 | 0 |
| Satb1 | 0 | 0 | 0 | 0 | 0 | 0 |
| Sdpr | 3.14060766 | 4.67747404 | 0 | 5.13849374 | 4.35123979 | 0 |
| Sell | 0 | 7.82142452 | 0 | 0 | 0 | 0 |
| Sfpi1 | 9.44004137 | 10.6112564 | 9.57177198 | 9.73952896 | 7.67485892 | 9.1636508 |
| Slamf1 | 0 | 9.8509578 | 0 | 7.94976735 | 0 | 0 |
| Smarca4 | 9.67242674 | 11.3679625 | 10.9120144 | 8.33633778 | 9.38747622 | 8.96597469 |
| Sos1 | 6.73189286 | 7.18014773 | 6.17729215 | 2.57292994 | 0 | 5.02443057 |
| Stat1 | 5.68555984 | 3.02264624 | 7.3271143 | 5.35339745 | 0.83073004 | 3.29153215 |
| Stat3 | 11.3131951 | 9.57939384 | 9.0893893 | 9.5064832 | 8.66288619 | 9.76664759 |
| Stat4 | 8.57556847 | 8.81788595 | 7.9582273 | 8.78864361 | 9.09957433 | 8.97134532 |
| Stat6 | 10.8376145 | 10.2010288 | 8.49312223 | 9.35277641 | 7.40643256 | 8.90732864 |
| Suz12 | 9.41780703 | 9.04550097 | 8.43918141 | 6.8443864 | 8.23939832 | 7.20948647 |
| Tal1 | 0.36745858 | 3.76022412 | 1.90443062 | 1.20031735 | 0 | 1.80499304 |
| Tcf3 | 0 | 11.0694031 | 10.6447268 | 0 | 0 | 0 |
| Tcf4 | 10.6969499 | 10.046866 | 9.90816861 | 9.69613559 | 10.4609901 | 8.34637629 |
| Tcf7 | 0 | 0 | 4.47895555 | 0 | 0 | 0.42028063 |
| Tek | 0 | 0 | 6.45746287 | 7.3858809 | 0 | 0 |
| Tfrc | 10.1334859 | 10.3260932 | 9.35672673 | 8.44227518 | 4.80666561 | 0 |
| Tgfb1 | 0 | 6.89665934 | 5.8186116 | 6.7222637 | 0.76900814 | 0 |
| Tgfb2 | 4.82881658 | 0 | 0 | 0 | 0 | 0 |
| Tgfb3 | 7.47747614 | 8.54176509 | 0 | 0 | 8.79652611 | 0 |
| Tnfrsf1a | 9.39147825 | 10.7935619 | 9.25501158 | 9.89472761 | 8.6880689 | 8.31560598 |
| Tnfrsf1b | 9.23838068 | 9.80031527 | 8.22142743 | 0 | 7.28814434 | 7.90865273 |
| Tnfrsf21 | 6.28949914 | 5.97728009 | 5.19350171 | 5.57452029 | 4.50016048 | 0 |
| Tnfsf10 | 7.22789618 | 7.36848679 | 6.75517567 | 7.16478253 | 0 | 7.06007861 |
| Tnfsf12 | 0 | 4.3060738 | 0 | 4.85239643 | 0 | 2.04680563 |
| Tob1 | 1.76727829 | 0 | 0 | 1.31719975 | 0 | 5.22971098 |
| vWF | 4.32082285 | 6.06693197 | 0 | 5.71670619 | 6.04694734 | 5.12273794 |
| Zbtb20 | 8.1961861 | 9.72922487 | 8.03051751 | 9.40713548 | 7.57941477 | 7.38691184 |
| Zbtb38 | 7.5702152 | 9.54076295 | 6.6697548 | 7.46445387 | 6.53307434 | 7.23318321 |
| Zfp532 | 0 | 0 | 0 | 0 | 5.04108384 | 5.18050424 |
| Zfp612 | 5.72138328 | 8.03852538 | 5.85275553 | 5.51442076 | 5.97875939 | 8.22421158 |
| Zfpm1 | 0 | 6.44875688 | 6.30023725 | 0 | 1.71015037 | 0 |
| Zhx2 | 9.12067496 | 8.78641727 | 0 | 8.48763196 | 0 | 0 |

TABLE 8-6
Single cell expression data (reduced list)-iHSC-8-TF-Poly

| | iHSC-8- | iHSC-8- | iHSC-8-TF- | iHSC-8-TF- | iHSC-8-TF- |
|---|---|---|---|---|---|
| Factor | TF-Poly31 | TF-Poly32 | Poly33 | Poly34 | Poly35 |
| Actb | 14.2069371 | 13.8470594 | 13.8401959 | 13.917789 | 15.1280325 |
| Aebp2 | 5.98889731 | 6.37700771 | 7.03385188 | 7.32807418 | 5.967507558 |
| Ahr | 0 | 7.35587653 | 7.14024783 | 0 | 7.726173885 |
| Ala1 | 9.63022936 | 9.79043235 | 8.92541514 | 10.255464 | 10.06829133 |
| Akt2 | 4.7739806 | 6.20050837 | 0 | 6.6173956 | 6.266455938 |
| Akt3 | 7.83294768 | 7.93223254 | 7.33454157 | 7.96075903 | 7.609211364 |
| APC | 7.03824303 | 2.01225823 | 7.3738631 | 6.86740225 | 0 |
| Bad | 0 | 0 | 0 | 0 | 0 |
| Bax | 7.92377163 | 9.35241369 | 8.79541456 | 10.1556033 | 9.298454044 |
| Bcl11a | 6.79087658 | 9.42268001 | 0 | 5.94056 | 9.650354382 |
| Bcl11b | 0 | 7.91342229 | 0 | 0 | 0 |
| Bcl2 | 7.71843033 | 4.37394315 | 3.67661636 | 6.48782736 | 6.12384282 |
| Bcl2l1 | 7.32275084 | 8.01987482 | 8.88727066 | 10.3391458 | 5.482050078 |
| Bcl2l11 | 5.79196834 | 7.61927617 | 0 | 0 | 7.954405054 |
| Bmi1 | 8.81392639 | 9.20924156 | 9.08236893 | 0 | 8.889304656 |
| Brd3 | 7.23409493 | 7.45401462 | 5.51991989 | 8.04268652 | 7.389789509 |
| Casp8 | 7.67563079 | 8.20820007 | 6.55654411 | 7.54337459 | 8.32215887 |
| Casp9 | 0 | 0 | 8.99779312 | 4.59384186 | 5.848587768 |
| Cbx2 | 1.44235903 | 5.10087886 | 3.11514136 | 4.33721335 | 7.198562206 |
| Cbx8 | 0 | 0 | 0 | 0 | 0 |
| Ccnc | 6.39235909 | 2.98958517 | 6.90788079 | 8.33600559 | 0 |
| Ccnd1 | 9.85365523 | 9.35220323 | 10.3423931 | 10.596546 | 10.3258133 |
| Ccne2 | 0 | 0 | 0 | 0 | 0 |
| CD34 | 8.18588751 | 7.74906415 | 7.26970785 | 0 | 7.499624637 |
| CD41 | 9.13809414 | 0 | 2.48229859 | 9.03163232 | 2.624405589 |
| CD48 | 0 | 0 | 0 | 0 | 0 |
| CD52 | 0 | 0 | 0 | 0 | 0 |
| CD53 | 9.41977885 | 9.65013579 | 7.29556871 | 0 | 7.157577428 |
| CD55 | 8.06965354 | 6.20993378 | 0 | 0 | 8.314622092 |
| CD63 | 8.2891293 | 8.70844929 | 8.28276973 | 7.95614666 | 7.974507291 |
| CD9 | 5.61055111 | 8.76259165 | 7.38090105 | 9.05799841 | 7.984779418 |
| Cdc42 | 11.6414373 | 11.5413516 | 11.8105407 | 12.0218361 | 12.15037822 |
| Cdk1 | 0 | 0 | 5.92738978 | 6.4822881 | 0 |
| Cdk4 | 9.11192333 | 8.53731642 | 7.38211559 | 9.25948872 | 8.7744804 |
| Cdkn2b | 0 | 2.02544167 | 0 | 0 | 0 |
| Cebpa | 0 | 0 | 0 | 1.76275336 | 0 |
| Csf1r | 0 | 0 | 0 | 0 | 0 |
| Ctnnb1 | 8.53778061 | 8.99449917 | 8.50354705 | 8.54550946 | 8.150621469 |
| Cycs | 8.74992664 | 9.3211739 | 7.56416714 | 11.6306877 | 9.73444361 |
| Dach1 | 10.2255054 | 8.13381132 | 9.01635767 | 9.67564058 | 9.403674066 |
| Dnmt1 | 7.06488647 | 8.20709121 | 5.48806315 | 10.7511069 | 9.291062883 |
| Dnmt3a | 9.34089662 | 10.5431275 | 9.68146699 | 9.67721509 | 9.574078858 |
| Dnmt3b | 7.10396864 | 7.14264453 | 0 | 8.67608269 | 8.398086808 |
| Dtx1 | 0 | 3.92664652 | 0 | 0 | 1.079050232 |
| Dtx4 | 0 | 0 | 6.49224019 | 0 | 7.288080256 |
| Ebf1 | 0 | 0 | 0 | 0 | 0 |
| Ep300 | 8.96510963 | 9.64835081 | 9.30091348 | 8.39112866 | 8.866505918 |
| Epor | 7.23361451 | 8.89683938 | 8.61954912 | 7.62063998 | 8.194140038 |
| Erg | 9.8355606 | 10.6000491 | 9.47258834 | 9.6821144 | 10.01801557 |
| Esr1 | 6.30347997 | 5.64608692 | 9.59441989 | 0 | 7.287947864 |
| ETS1 | 6.09111489 | 6.98717296 | 7.45969571 | 6.50362082 | 5.966052941 |
| ETS2 | 8.21354447 | 9.19096881 | 1.88892339 | 8.82189923 | 7.475011402 |
| Etv3 | 2.96178532 | 5.48992927 | 4.14441284 | 0 | 2.046570736 |
| Etv6 | 10.858902 | 10.7925323 | 9.17798475 | 10.4215528 | 8.441479121 |
| Ezh2 | 0 | 0 | 0 | 5.45401289 | 5.720754812 |
| Fas | 0 | 0 | 0 | 0 | 0 |
| Fcgr2b | 6.88075674 | 5.54617113 | 7.14891342 | 0 | 5.207646663 |
| Fcgr3 | 0 | 7.00826514 | 2.35530291 | 0 | 0 |
| Fli1 | 10.4691328 | 10.6639924 | 10.8692473 | 10.8916346 | 11.73795664 |
| Flt3 | 7.70280609 | 0 | 0 | 0 | 0 |
| Fosl1 | 0 | 0 | 0 | 7.93834953 | 0 |
| Foxo1 | 9.88312231 | 9.91869001 | 9.41788508 | 9.980959 | 9.032259159 |
| Foxo3 | 8.9575813 | 9.11938907 | 5.14307614 | 6.31778497 | 8.269683905 |
| Gapdh | 7.77415605 | 8.26157075 | 7.66942984 | 7.99106849 | 8.407711578 |
| Gata1 | 0 | 0 | 0 | 9.41402438 | 0 |
| Gata2 | 7.15464892 | 7.34496127 | 8.49543986 | 7.32606855 | 6.666698464 |
| Gata3 | 8.87820207 | 6.20110618 | 8.30533556 | 6.87821567 | 10.13942411 |
| Gfi1 | 3.66002454 | 10.3420315 | 3.3332712 | 0 | 9.13521169 |
| Gfi1b | 9.7202357 | 0 | 7.68331245 | 7.60509615 | 0 |
| Hes5 | 0 | 0 | 0 | 0 | 0 |
| Hey1 | 0 | 0 | 0 | 0.16937746 | 0 |
| Hlf | 9.6974979 | 11.3333543 | 10.0480033 | 5.45401024 | 10.71087925 |
| Id2 | 0 | 1.73028986 | 2.9821102 | 0 | 0 |
| Ifi203 | 11.0839141 | 12.322849 | 11.1353627 | 0 | 11.36781538 |
| Ifi205 | 0 | 0.60415365 | 0 | 0 | 0 |
| Ifitm1 | 11.7471245 | 14.7096222 | 13.8201159 | 10.3244566 | 12.69088657 |
| Ikzf1 | 4.65390199 | 9.36172059 | 8.32379103 | 9.23805477 | 9.117655761 |
| Ikzf2 | 8.1800366 | 9.34772631 | 8.67384155 | 7.5997184 | 7.633862356 |
| Il7R | 0 | 0 | 0 | 0 | 0 |
| Irf4 | 0 | 0 | 0 | 0 | 0 |
| Irf6 | 4.55947022 | 0 | 6.20746303 | 1.90705149 | 0 |
| Irf8 | 4.17972502 | 5.19690275 | 0 | 0 | 1.168908887 |
| Kdr | 0 | 6.4546143 | 0 | 0 | 0 |
| Kit | 10.568118 | 10.7326629 | 9.66035824 | 10.235182 | 10.49875083 |
| Klf1 | 0 | 0 | 0 | 0 | 0 |
| Klf12 | 7.90580132 | 8.82046599 | 6.9604333 | 0 | 4.799978797 |
| Ldb1 | 10.1435572 | 10.8500431 | 9.02813399 | 10.0262901 | 10.57537202 |
| Lin28a | 2.01835712 | 0 | 7.60463245 | 7.26719796 | 7.0567702 |
| Lmo2 | 10.2921708 | 10.7989969 | 10.2263373 | 10.2137336 | 10.88135035 |
| Ly6a | 9.83101715 | 10.8834217 | 10.9471589 | 7.0165923 | 8.632926147 |
| Lyl1 | 5.64299125 | 8.39061157 | 8.05543657 | 0 | 6.93753767 |
| Mbd2 | 7.43457425 | 9.26608484 | 9.03952586 | 9.2082395 | 8.436864241 |
| Meis1 | 7.17124275 | 8.43708101 | 8.22260057 | 9.10089289 | 9.11887556 |
| Mllt3 | 4.68195721 | 7.14795197 | 0 | 0 | 0 |
| Mpl | 8.08112178 | 11.2655793 | 8.31206648 | 8.38505498 | 10.42540532 |
| Muc13 | 7.69684641 | 8.740324 | 7.23849573 | 9.18630096 | 9.032771705 |
| Myb | 10.8533459 | 12.0368698 | 11.2785518 | 11.5385888 | 11.07370836 |
| Myc | 10.6275942 | 0 | 7.98150016 | 10.2477795 | 0 |
| Mycn | 11.5919441 | 13.7950767 | 13.0318118 | 11.572173 | 9.884323219 |
| Ndn | 7.40457998 | 10.7183908 | 8.75991643 | 0 | 9.66450548 |
| Nfat5 | 9.91314435 | 11.3027566 | 10.3793203 | 9.5179896 | 9.779535652 |
| Nfia | 7.79221189 | 10.051871 | 9.80645605 | 8.5847577 | 9.825414289 |
| Nfkb1 | 5.30418245 | 4.7738356 | 0 | 2.24245044 | 0 |
| Notch1 | 0 | 0 | 8.15000269 | 0 | 7.573067604 |
| Pax4 | 0 | 0 | 0 | 0 | 0 |
| Pax5 | 6.27471676 | 0 | 0 | 0 | 0 |
| Pax9 | 0 | 0 | 0 | 0 | 0 |
| Pbx1 | 2.98370939 | 0 | 5.09401103 | 0 | 4.388365504 |
| PIk3ca | 8.75259112 | 8.2331466 | 9.04017873 | 8.40060398 | 8.480971265 |
| PIk3R2 | 5.26485409 | 8.45942473 | 7.82585643 | 7.39316382 | 9.393048953 |
| Plag1 | 0 | 7.30362382 | 0 | 7.34771911 | 9.10689314 |
| Prf1 | 0 | 1.7040015 | 1.56236886 | 0 | 0 |
| Pten | 8.31345133 | 10.2695925 | 9.97217513 | 9.13715083 | 10.61329076 |
| Rb1 | 9.95763649 | 9.79666893 | 7.99445736 | 9.2214006 | 9.482724273 |
| Rora | 0 | 6.56427305 | 5.64107699 | 4.56057422 | 6.331115465 |
| Runx1 | 0 | 8.14608613 | 8.71941715 | 8.19287364 | 7.870521822 |
| Runx2 | 5.34834113 | 4.59771607 | 3.47214128 | 0 | 5.551463025 |
| Satb1 | 0 | 0 | 0 | 0 | 0 |
| Sdpr | 3.37648888 | 5.96772223 | 4.92952477 | 6.2586625 | 0.440922771 |
| Sell | 0 | 0 | 0 | 0 | 0 |
| Sfpi1 | 8.60729629 | 8.1629534 | 8.63327996 | 0 | 9.702906322 |
| Slamf1 | 8.18217953 | 8.5258681 | 9.31528985 | 0 | 0 |
| Smarca4 | 9.34699951 | 9.96472508 | 9.88851572 | 10.4282604 | 9.608738237 |
| Sos1 | 5.09407549 | 6.03558245 | 5.61700797 | 6.34922122 | 5.074256436 |
| Stat1 | 1.59542643 | 3.04779271 | 4.03139248 | 2.14901292 | 1.713356031 |
| Stat3 | 9.68231911 | 10.8000054 | 9.77728389 | 8.12814266 | 9.356540529 |
| Stat4 | 9.03078852 | 8.81875947 | 9.7628284 | 9.23753873 | 9.345223627 |
| Stat6 | 8.80712329 | 10.4891182 | 8.86298599 | 9.36542993 | 9.506915587 |
| Suz12 | 9.14292326 | 7.20766953 | 7.75046294 | 8.75372748 | 8.5960251 |
| Tal1 | 2.01130091 | 0.68276644 | 6.73023432 | 2.3305105 | 3.375132509 |
| Tcf3 | 10.2607205 | 0 | 10.2753363 | 0 | 9.495266453 |
| Tcf4 | 9.35302065 | 9.01808097 | 10.5037967 | 9.43819789 | 10.19593089 |
| Tcf7 | 0 | 0 | 0 | 0 | 2.167615009 |
| Tek | 0 | 7.96828571 | 0 | 0 | 0 |
| Tfrc | 10.289075 | 9.33491809 | 0 | 8.98476872 | 8.421750863 |
| Tgfb1 | 0 | 0 | 1.58449749 | 3.70605263 | 0 |
| Tgfb2 | 0 | 6.77707617 | 0 | 3.27306737 | 0 |
| Tgfb3 | 7.58607476 | 0 | 0 | 0 | 0 |
| Tnfrsf1a | 10.1591524 | 9.83805082 | 9.98992194 | 8.08094003 | 9.174520259 |
| Tnfrsf1b | 9.13727 | 7.96181671 | 7.64996091 | 8.33642919 | 8.933678342 |
| Tnfrsf21 | 5.11707811 | 5.6723159 | 2.99946137 | 5.63253583 | 4.286614066 |
| Tnfsf10 | 4.56651794 | 7.36235451 | 6.96023501 | 0 | 0 |
| Tnfsf12 | 0 | 0 | 0 | 0 | 0 |
| Tob1 | 0 | 7.90731485 | 0 | 5.34587986 | 6.280356776 |
| vWF | 5.50820839 | 7.20387901 | 7.86788155 | 7.34146511 | 6.367927725 |
| Zbtb20 | 9.56374662 | 9.76898114 | 9.31718552 | 6.40168708 | 9.092322576 |
| Zbtb38 | 7.131271 | 8.33887914 | 7.67573354 | 5.98684877 | 7.925976084 |
| Zfp532 | 0 | 0 | 0 | 0 | 0 |
| Zfp612 | 7.37417759 | 7.21875833 | 8.50575865 | 7.70407891 | 7.488313843 |
| Zfpm1 | 0 | 0 | 0 | 0 | 0 |
| Zhx2 | 8.5972312 | 0 | 8.15875098 | 0 | 7.312382961 |

### REFERENCES FOR EXAMPLE 2

Bock, C., Beerman, I., Lien, W. H., Smith, Z. D., Gu, H., Boyle, P., Gnirke, A., Fuchs, E., Rossi, D. J., and Meissner, A. (2012). Mol Cell 47, 633-647.
Boehm, T., Foroni, L., Kaneko, Y., Perutz, M. F., and Rabbitts, T. H. (1991). Proc Natl Acad Sci USA 88, 4367-4371.
Briggs, R., and King, T. J. (1952). Proc Natl Acad Sci USA 38, 455-463.
Brodeur, G. M., Seeger, R. C., Schwab, M., Varmus, H. E., and Bishop, J. M. (1984). Science 224, 1121-1124.
Campbell, K. H., McWhir, J., Ritchie, W. A., and Wilmut, I. (1996). Nature 380, 64-66.
Choi, J., Costa, M. L., Mermelstein, C. S., Chagas, C., Holtzer, S., and Holtzer, H. (1990). Proc Natl Acad Sci USA 87, 7988-7992.
Choi, K. D., Vodyanik, M. A., and Slukvin, II (2009). J Clin Invest 119, 2818-2829.
Cobaleda, C., Jochum, W., and Busslinger, M. (2007). Nature 449, 473-477.
Copelan, E. A. (2006). N Engl J Med 354, 1813-1826.
Davis, R. L., Weintraub, H., and Lassar, A. B. (1987). Cell 51, 987-1000.
Doulatov, S., Vo, L. T., Chou, S. S., Kim, P. G., Arora, N., Li, H., Hadland, B. K., Bernstein, I. D., Collins, J. J., Zon, L. I., et al. (2013) Cell Stem Cell 13, 459-470.
Erickson, P., Gao, J., Chang, K. S., Look, T., Whisenant, E., Raimondi, S., Lasher, R., Trujillo, J., Rowley, J., and Drabkin, H. (1992). Blood 80, 1825-1831.
Ficara, F., Murphy, M. J., Lin, M., and Cleary, M. L. (2008). Cell Stem Cell 2, 484-496.
Garcon, L., Ge, J., Manjunath, S. H., Mills, J. A., Apicella, M., Parikh, S., Sullivan, L. M., Podsakoff, G. M., Gadue, P., French, D. L., et al. (2013). Blood 122, 912-921.
Gazit, R., Garrison, B. S., Rao, T. N., Shay, T., Costello, J. F., Erikson, J., Collins, J. J., Regev, A., Wagers, A., and Rossi, D. J. (2013) Stem Cell Reports Vol. 1, issue 3, 266-280.
Gratwohl, A., Baldomero, H., Aljurf, M., Pasquini, M. C., Bouzas, L. F., Yoshimi, A., Szer, J., Lipton, J., Schwendener, A., Gratwohl, M., et al. (2010). JAMA 303, 1617-1624.
Guo, G., Luc, S., Marco, E., Lin, T. W., Peng, C., Kerenyi, M. A., Beyaz, S., Kim, W., Xu, J., Das, P. P., et al. (2013). Cell Stem Cell.
Gurdon, J. B., and Uehlinger, V. (1966). Nature 210, 1240-1241.
Hanna, J., Markoulaki, S., Schorderet, P., Carey, B. W., Beard, C., Wernig, M., Creyghton, M. P., Steine, E. J., Cassady, J. P., Foreman, R., et al. (2008). Cell 133, 250-264.
Hisa, T., Spence, S. E., Rachel, R. A., Fujita, M., Nakamura, T., Ward, J. M., Devor-Henneman, D. E., Saiki, Y., Kutsuna, H., Tessarollo, L., et al. (2004). Embo J 23, 450-459.
Ieda, M., Fu, J. D., Delgado-Olguin, P., Vedantham, V., Hayashi, Y., Bruneau, B. G., and Srivastava, D. (2010). Cell 142, 375-386.
Inaba, T., Roberts, W. M., Shapiro, L. H., Jolly, K. W., Raimondi, S. C., Smith, S. D., and Look, A. T. (1992). Science 257, 531-534.
Iwasaki, H., Mizuno, S., Arinobu, Y., Ozawa, H., Mori, Y., Shigematsu, H., Takatsu, K., Tenen, D. G., and Akashi, K. (2006). Genes Dev 20, 3010-3021.
Kamps, M. P., Look, A. T., and Baltimore, D. (1991). Genes Dev 5, 358-368.
Kennedy, M., Awong, G., Sturgeon, C. M., Ditadi, A., LaMotte-Mohs, R., Zuniga-Pflucker, J. C., and Keller, G. (2012). Cell reports 2, 1722-1735.
Kocabas, F., Zheng, J., Thet, S., Copeland, N. G., Jenkins, N. A., Deberardinis, R. J., Zhang, C., and Sadek, H. A. (2012). Blood.
Kondo, M., Scherer, D. C., Miyamoto, T., King, A. G., Akashi, K., Sugamura, K., and Weissman, I. L. (2000). Nature 407, 383-386.
Kubota, Y., Osawa, M., Jakt, L. M., Yoshikawa, K., and Nishikawa, S. (2009). Blood 114, 4383-4392.
Laiosa, C. V., Stadtfeld, M., Xie, H., de Andres-Aguayo, L., and Graf, T. (2006). Immunity 25, 731-744.
Laurenti, E., Varnum-Finney, B., Wilson, A., Ferrero, I., Blanco-Bose, W. E., Ehninger, A., Knoepfler, P. S., Cheng, P. F., et al. (2008). Cell Stem Cell 3, 611-624.
Mali, P., Ye, Z., Hommond, H. H., Yu, X., Lin, J., Chen, G., Zou, J., and Cheng, L. (2008). Stem Cells 26, 1998-2005.
Marx, J. L. (1984). The N-myc oncogene in neural tumors. Science 224, 1088.
McCormack, M. P., Young, L. F., Vasudevan, S., de Graaf, C. A., Codrington, R., Rabbitts, T. H., Jane, S. M., and Curtis, D. J. (2010). Science 327, 879-883.
Min, I. M., Pietramaggiori, G., Kim, F S, Passegue, E., Stevenson, K. E., and Wagers, A. J. (2008). Cell Stem Cell 2, 380-391.
Moskow, J. J., Bullrich, F., Huebner, K., Daar, I. O., and Buchberg, A. M. (1995). Mol Cell Biol 15, 5434-5443.
Mostoslaysky, G., Kotton, D. N., Fabian, A. J., Gray, J. T., Lee, J. S., and Mulligan, R. C. (2005). Mol Ther 11, 932-940.
Muller, L. U., Milsom, M. D., Harris, C. E., Vyas, R., Brumme, K. M., Parmar, K., Moreau, L. A., Schambach, A., Park, I. H., London, W. B., et al. (2012). Blood 119, 5449-5457.
Najm, F. J., Lager, A. M., Zaremba, A., Wyatt, K., Caprariello, A. V., Factor, D. C., Karl, R. T., Maeda, T., Miller, R. H., and Tesar, P. J. (2013). Nat Biotechnol 31, 426-433.
Niu, W., Zang, T., Zou, Y., Fang, S., Smith, D. K., Bachoo, R., and Zhang, C. L. (2013). Nat Cell Biol 15, 1164-1175.
Okita, K., Ichisaka, T., and Yamanaka, S. (2007 Nature 448, 313-317.
Orkin, S. H., and Zon, L. I. (2008). Cell 132, 631-644.
Pang, Z. P., Yang, N., Vierbuchen, T., Ostermeier, A., Fuentes, D. R., Yang, T. Q., Citri, A., Sebastiano, V., Marro, S., Sudhof, T. C., et al. (2011). Nature 476, 220-223.
Pereira, C. F., Chang, B., Qiu, J., Niu, X., Papatsenko, D., Hendry, C. E., Clark, N. R., Nomura-Kitabayashi, A., Kovacic, J. C., Ma'ayan, A., et al. (2013). Cell Stem Cell.
Polo, J. M., Liu, S., Figueroa, M. E., Kulalert, W., Eminli, S., Tan, K. Y., Apostolou, E., Stadtfeld, M., Li, Y., Shioda, T., et al. (2010 Nat Biotechnol 28, 848-855.
Qian, L., Huang, Y., Spencer, C. I., Foley, A., Vedantham, V., Liu, L., Conway, S. J., Fu, J. D., and Srivastava, D. (2012). Nature 485, 593-598.
Song, K., Nam, Y. J., Luo, X., Qi, X., Tan, W., Huang, G. N., Acharya, A., Smith, C. L., Tallquist, M. D., Neilson, E. G., et al. (2012). Nature 485, 599-604.
Sturgeon, C. M., Ditadi, A., Clarke, R. L., and Keller, G. (2013). Nat Biotechnol 31, 416-418.
Szabo, E., Rampalli, S., Risueno, R. M., Schnerch, A., Mitchell, R., Fiebig-Comyn, A., Levadoux-Martin, M., and Bhatia, M. (2010). Nature 468, 521-526.
Taghon, T., Yui, M. A., and Rothenberg, E. V. (2007). Nat Immunol 8, 845-855.
Takahashi, K., Tanabe, K., Ohnuki, M., Narita, M., Ichisaka, T., Tomoda, K., and Yamanaka, S. (2007). Cell 131, 861-872.
Takahashi, K., and Yamanaka, S. (2006). Cell 126, 663-676.
Tulpule, A., Kelley, J. M., Lensch, M. W., McPherson, J., Park, I. H., Hartung, O., Nakamura, T., Schlaeger, T. M., Shimamura, A., and Daley, G. Q. (2013). Cell Stem Cell 12, 727-736.
Unnisa, Z., Clark, J. P., Roychoudhury, J., Thomas, E., Tessarollo, L., Copeland, N. G., Jenkins, N. A., Grimes, H. L., and Kumar, A. R. (2012). Blood 120, 4973-4981.
Vierbuchen, T., Ostermeier, A., Pang, Z. P., Kokubu, Y., Sudhof, T. C., and Wernig, M. (2010). Direct conversion of fibroblasts to functional neurons by defined factors. Nature 463, 1035-1041.
Warren, A. J., Colledge, W. H., Carlton, M. B., Evans, M. J., Smith, A. J., and Rabbitts, T. H. (1994). Cell 78, 45-57.
Winkler, T., Hong, S. G., Decker, J. E., Morgan, M. J., Wu, C., Hughes, W. M. t., Yang, Y., Wangsa, D., Padilla-Nash, H. M., Ried, T., et al. (2013). J Clin Invest 123, 1952-1963.
Xie, H., Ye, M., Feng, R., and Graf, T. (2004). Cell 117, 663-676.
Yamada, Y., Warren, A. J., Dobson, C., Forster, A., Pannell, R., and Rabbitts, T. H. (1998). Proc Natl Acad Sci USA 95, 3890-3895.
Yang, N., Zuchero, J. B., Ahlenius, H., Marro, S., Ng, Y. H., Vierbuchen, T., Hawkins, J. S., Geissler, R., Barres, B. A., and Wernig, M. (2013). Nat Biotechnol 31, 434-439.
Ye, L., Chang, J. C., Lin, C., Sun, X., Yu, J., and Kan, Y. W. (2009). Proc Natl Acad Sci USA 106, 9826-9830.
Yuasa, H., Oike, Y., Iwama, A., Nishikata, I., Sugiyama, D., Perkins, A., Mucenski, M. L., Suda, T., and Morishita, K. (2005). Embo J 24, 1976-1987.
Zhou, Q., et al. (2008). Nature 455, 627-632.
Zhou, Q., and Melton, D. A. (2008). Cell Stem Cell 3, 382-388.

### Example 3

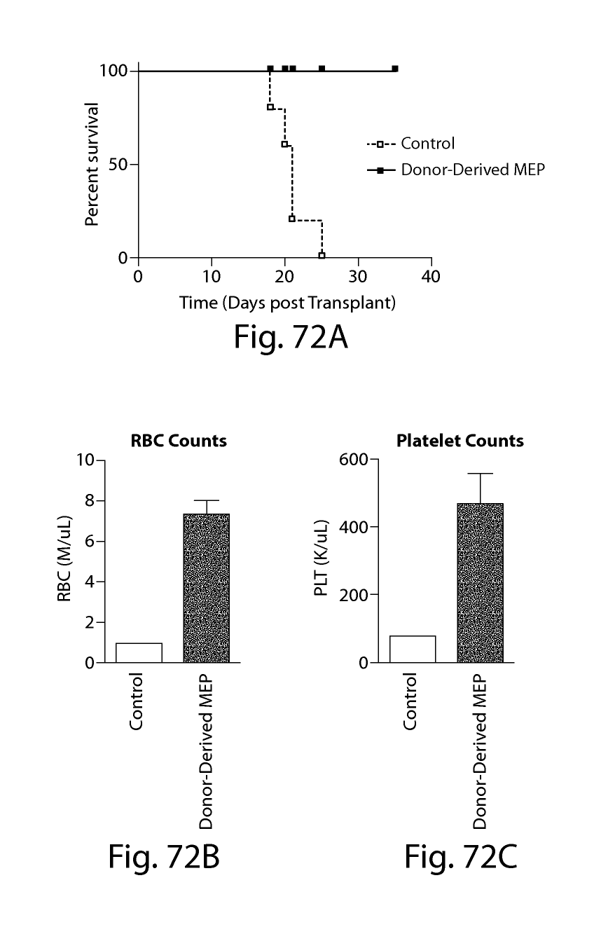

Radioprotection transplantation assays performed using donor-derived MEPs (Na Nakorn, J Clin Invest. 2002, 109(12), 1579-85) confirmed a robust ability to give rise to platelets and red blood cells in vivo (FIGS. 72B-C).

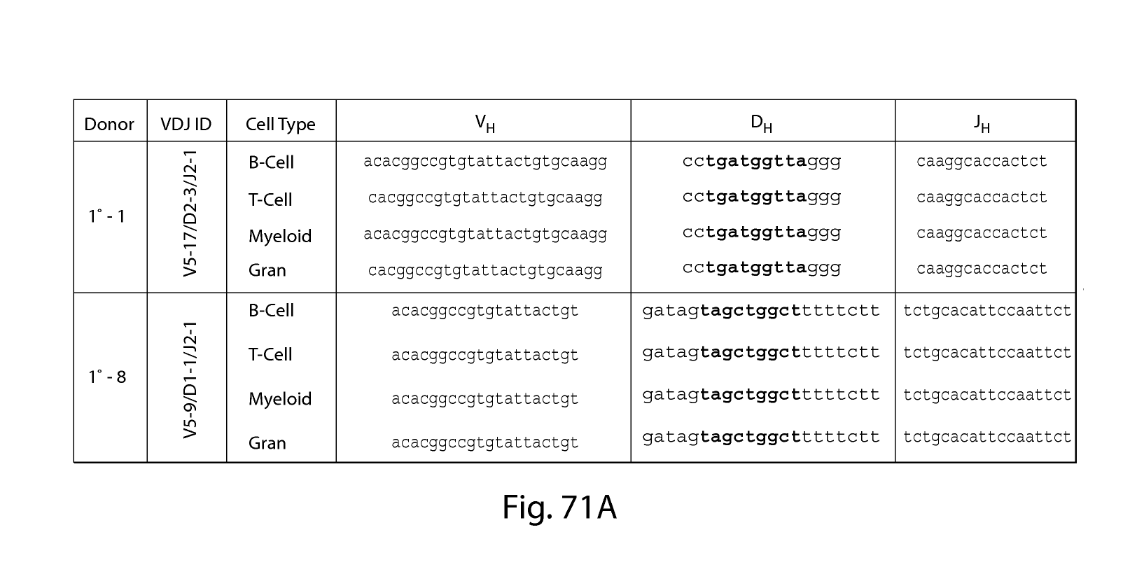

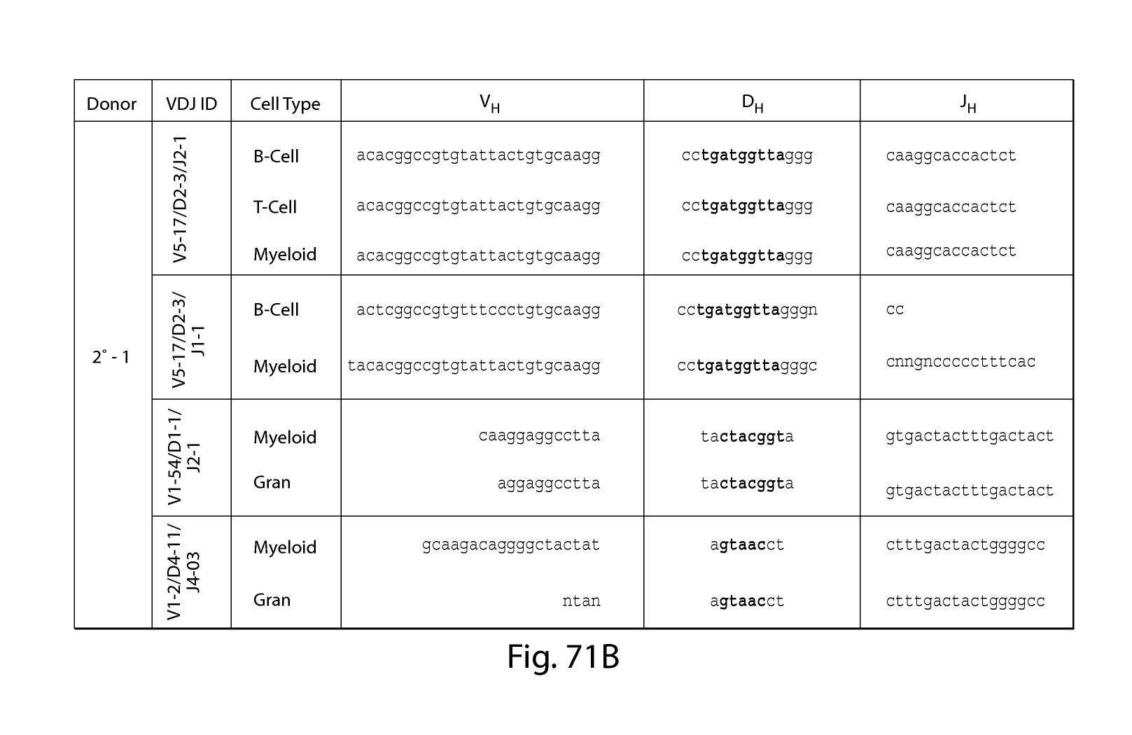

In addition to sustained self-renewal potential, a hallmark property of HSCs is their ability to give rise to multi-lineage differentiation at the clonal level. Although we had observed clonal multi-lineage differentiation potential in vitro after induction of our factors (FIGS. 60B-C), our in vivo transplantation experiments, which were done at the population level, precluded us from concluding clonal differentiation potential in vivo. We reasoned that Ig heavy chain rearrangements arising in Pro/Pre B-cells could be used as a lineage-tracing tool, and that the presence of common V(D)J rearrangements in different donor-derived lineages in our transplantation experiments could provide evidence of clonal multi-lineage differentiation potential. We therefore isolated DNA from sorted donor-derived B- and T-cells, granulocytes, and macrophage/monocytes from primary recipients exhibiting long-term multi-lineage reconstitution derived from Pro/Pre B-cells transduced with the 8-TF^{Poly} viral cocktail. Ig heavy chain-specific PCR spanning the V(D)J junction was then performed and selected products common in size to all lineages were gel purified, cloned and sequenced. This analysis revealed the presence of V(D)J rearrangements common to all of the donor-derived lineages we analyzed from two independent experiments, indicating multi-lineage differentiation potential from clonal reprogrammed Pro/Pre B-cells (FIG. 71A). That single reprogrammed cells possessed multi-lineage differentiation potential in vivo was further confirmed using a LAM-PCR-based approach, which revealed common viral integration sites in sorted donor-derived B-, T-, and myeloid cells (not shown). To test if reprogrammed cells sustained multi-lineage differentiation capacity during serial transplantation, we analyzed V(D)J junctions and viral integration via LAM-PCR on sorted donor-derived B-, T- and myeloid cells from secondary recipients transplanted with reprogrammed cells from mice that had been analyzed by these approaches during primary transplantation. These experiments revealed that both shared V(D)J rearrangements and common viral integration sites could be identified in multiple lineages in both primary and secondary recipients (FIGS. 71B-71C), indicating that single reprogrammed cells possessed both multi-lineage differentiation, and long-term self-renewal potential.

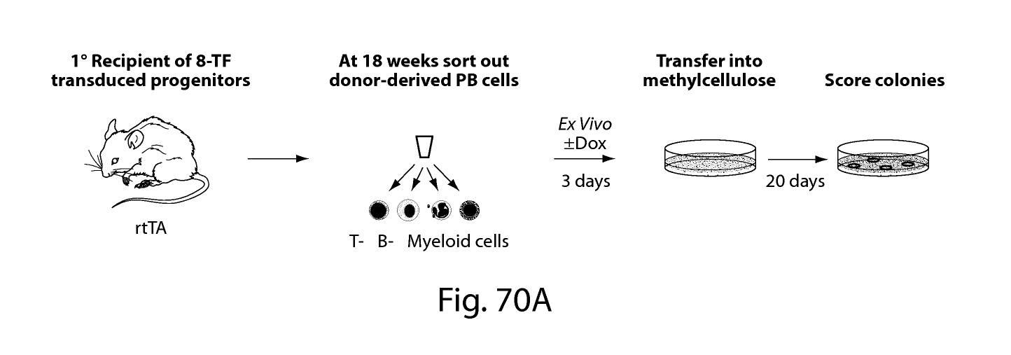

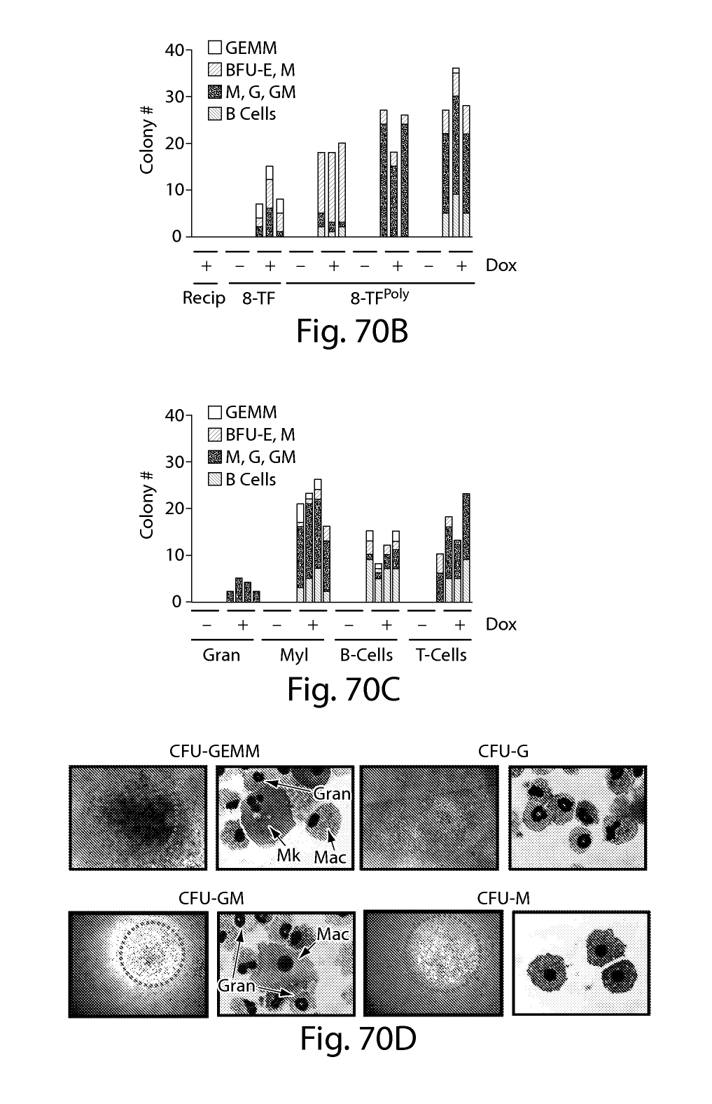

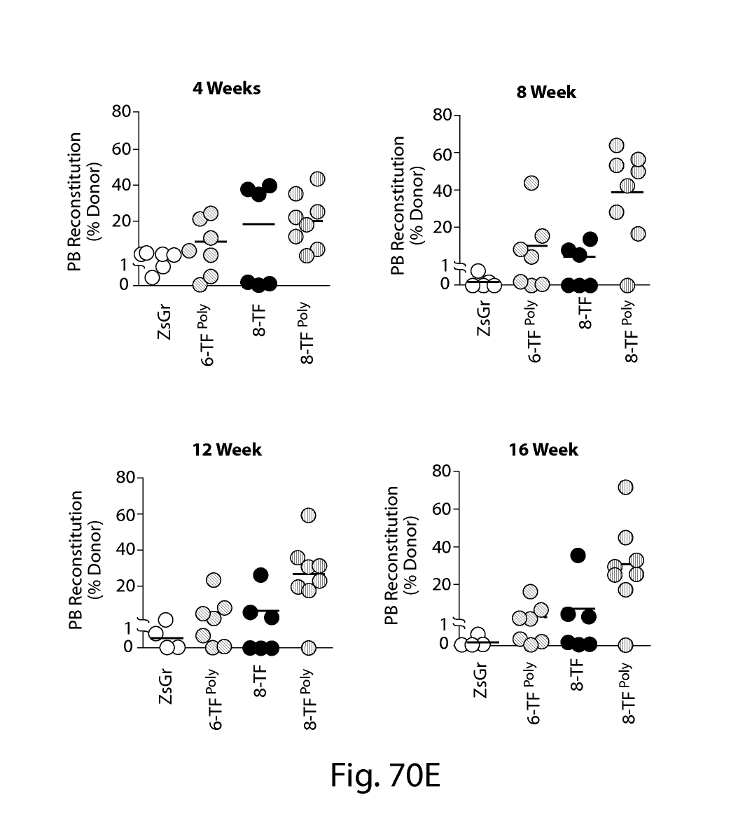

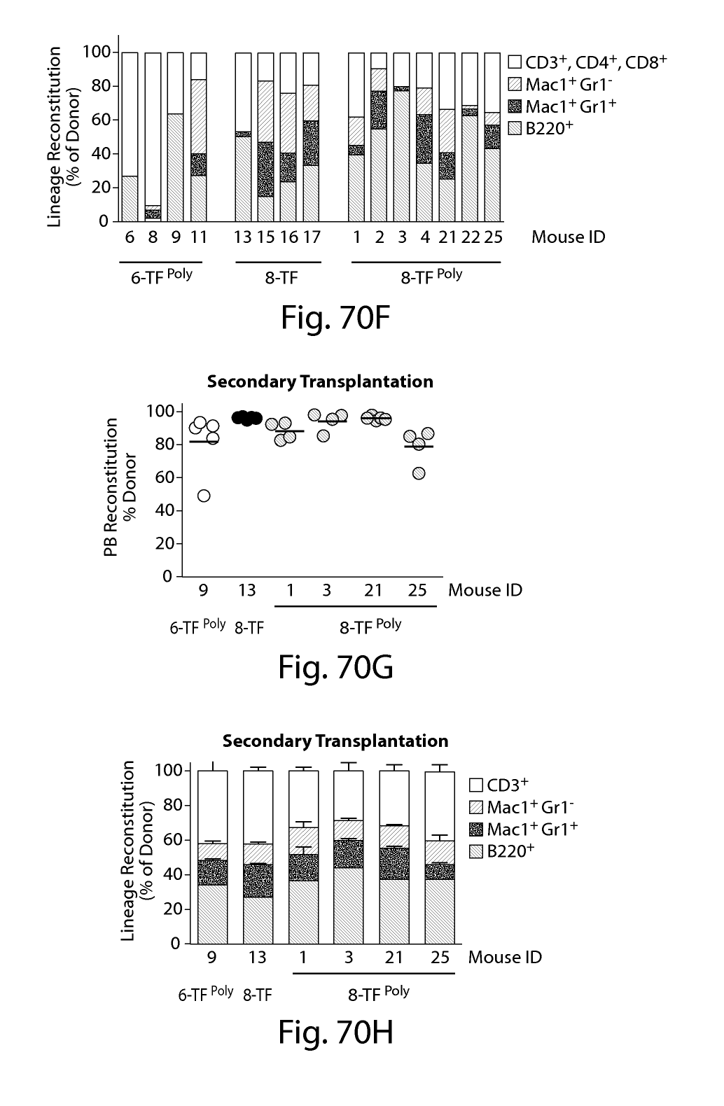

To determine which lineage(s) in the peripheral blood had the potential to give rise to these colonies upon re-expression of the transcription factors, we purified B-cells, T-cells, myeloid cells and granulocytes from the 8-TF^{Poly} reconstituted mice, and tested their colony forming potential following culturing and plating in the absence or presence of doxycycline. These experiments revealed that cells from each of these lineages were imbued with progenitor activity upon factor re-induction. Of these, granulocytes gave rise to the fewest colonies whereas Mac1+ macrophages/monocytes yielded the largest number of colonies and the greatest number of primitive GEMM colonies (FIGS. 70C-D).

We focused on differentiated myeloid cells because unlike differentiated lymphoid cells that have rearranged TCR (T-cells) or IG (B-cells) loci, multi-lineage reconstituting cells derived via reprogramming of myeloid cells would be expected to have the potential to give rise to full repertoires of lymphoid effector cells upon differentiation.

1. A hematopoietic stem cell (HSC) inducing composition comprising one or more expression vector encoding four or more HSC inducing factors selected from: CDKN1C, DNMT3B, EGR1, ETV6, EVI1, GATA2, GFI1B, GLIS2, HLF, HMGA2, HOXA5, HOXA9, HOXB3, HOXB4, HOXB5, IGF2BP2, IKZF2, KLF12, KLF4, KLF9, LMO2, MEIS1, MSI2, MYCN, NAP1L3, NDN, NFIX, NKX2-3, NR3C2, PBX1, PRDM16, PRDM5, RARB, RBBP6, RBPMS, RUNX1, RUNX1T1, SMAD6, TAL1, TCF15, VDR, ZFP37, ZFP467, ZFP521, ZFP532, ZFP612, and ZPF467.

2-47. (canceled)

48. The HSC inducing composition of claim 1, wherein the four or more HSC inducing factors are HLF, RUNX1T1, PBX1, LMO2, PRDM5, ZFP37, MYCN, MSI2, NKX2-3, MEIS1, and RBPMS.

49. The HSC inducing composition of claim 1, wherein the four or more HSC inducing factors are HLF, RUNX1T1, ZFP37, PBX1, LMO2, and PRDM5.

50. The HSC inducing compositions of claim 1, wherein the expression vector is a retroviral vector.

51. The HSC inducing compositions of claim 1, wherein the expression vector is a lentiviral vector.

52. The HSC inducing composition of claim 51, wherein the lentiviral vector is an inducible lentiviral vector.

53. A method for preparing an induced hematopoietic stem cell (iHSC) from a somatic cell comprising:
a. transducing the somatic cell with one or more vectors comprising a nucleic acid sequence encoding HLF, a nucleic acid sequence encoding RUNX1T1; a nucleic acid sequence encoding ZFP37; a nucleic acid sequence encoding PBX1; a nucleic acid sequence encoding LMO2; and a nucleic acid sequence encoding PRDM5, wherein each said nucleic acid sequence is operably linked to a promoter; and
b. culturing the transduced somatic cell in a cell media that supports growth of hematopoietic stem cells, thereby preparing an iHSC.

54. The method of claim 53, wherein the transducing of step (a) further comprises one or more vectors comprising one or more of: a nucleic acid sequence encoding PRDM16 a nucleic acid sequence encoding ZFP467; and a nucleic acid sequence encoding VDR wherein each said nucleic acid sequence is operably linked to a promoter.

55. The method of claim 53 further comprising transducing the somatic cell with one or more vectors comprising a nucleic acid sequence encoding ZFP37; a nucleic acid sequence encoding MYCN; a nucleic acid sequence encoding MSI2; a nucleic acid sequence encoding NKX2-3; a nucleic acid sequence encoding MEIS1; and a nucleic acid sequence encoding RBPMS; wherein each said nucleic acid sequence is operably linked to a promoter.

56. The method of claim 53, wherein the expression vector is a retroviral vector.

57. The method of claim 53, wherein the expression vector is a lentiviral vector.

58. The method of claim 57, wherein the lentiviral vector is an inducible lentiviral vector.

59. The method of claim 53, wherein the somatic cell is a fibroblast cell.

60. The method of claim 53, wherein the somatic cell is a hematopoietic lineage cell.

61. The method of claim 60, wherein the hematopoietic lineage cell is selected from promyelocytes, neutrophils, eosinophils, basophils, reticulocytes, erythrocytes, mast cells, osteoclasts, megakaryoblasts, platelet producing megakaryocytes, platelets, monocytes, macrophages, dendritic cells, lymphocytes, NK cells, NKT cells, innate lymphocytes, multipotent hematopoietic progenitor cells, oligopotent hematopoietic progenitor cells, and lineage restricted hematopoietic progenitors.

62. The method of claim 60, wherein the hematopoietic lineage cell is selected from a multi-potent progenitor cell (MPP), common myeloid progenitor cell (CMP), granulocyte-monocyte progenitor cells (GMP), common lymphoid progenitor cell (CLP), and pre-megakaryocyte-erythrocyte progenitor cell.

63. The method of claim 60, wherein the hematopoietic lineage cell is selected from a megakaryocyte-erythrocyte progenitor cell (MEP), a ProB cell, a PreB cell, a PreProB cell, a ProT cell, a double-negative T cell, a pro-NK cell, a pro-dendritic cell (pro-DC), pre-granulocyte/macrophage cell, a granulocyte/macrophage progenitor (GMP) cell, and a pro-mast cell (ProMC).

64. A method of promoting transdifferentiation of a ProPreB cell to the myeloid lineage comprising:
a. transducing a ProPreB cell with one or more vectors comprising a nucleic acid sequence encoding ZFP467, a nucleic acid sequence encoding PBX1; a nucleic acid sequence encoding HOXB4; and a nucleic acid sequence encoding MSI2; wherein each said nucleic acid sequence is operably linked to a promoter; and
b. culturing the transduced ProPreB cell in a cell media that supports growth of myeloid lineage cells, thereby transdifferentiating the ProPreB cell to the myeloid lineage.

65. The method of claim 64, wherein the transducing of step (a) further comprises one or more vectors comprising one or more of: a nucleic acid sequence encoding HLF, a nucleic acid sequence encoding LMO2; a nucleic acid sequence encoding PRDM16; and a nucleic acid sequence encoding ZFP37.

66. The method of claim 63, wherein the expression vector is a retroviral vector.

67. The method of claim 63, wherein the expression vector is a lentiviral vector.

68. A kit for making induced hematopoietic stem cells (iHSCs) comprising the HSC inducing compositions of claim 1.
